(19) United States
(12) Patent Application Publication
(10) Pub. No.: US 2025/0223281 A1
(43) Pub. Date: Jul. 10, 2025

(54) ANTIDIABETIC COMPOUNDS AND COMPOSITIONS

(71) Applicant: REZUBIO PHARMACEUTICALS CO., LTD, Zhuhai, Guangdong (CN)

(72) Inventors: Hongping GUAN, Scotch Plains, NJ (US); Yusheng XIONG, Plainsboro, NJ (US)

(21) Appl. No.: 18/728,541

(22) PCT Filed: Jan. 12, 2023
(86) PCT No.: PCT/CN2023/071833

§ 371 Date: Jul. 12, 2024

(30) Foreign Application Priority Data
Jan. 14, 2022 (WO) PCT/CN2022/071974

(51) Int. Cl. C07D 405/14; A61K 31/4192; A61K 31/454; A61K 31/4545; A61K 31/4995; C07D 249/04; C07D 401/14; C07D 487/08

(52) U.S. Cl. CPC C07D 405/14; A61K 31/4192; A61K 31/454; A61K 31/4545; A61K 31/4995; C07D 249/04; C07D 401/14; C07D 487/08

(57) ABSTRACT

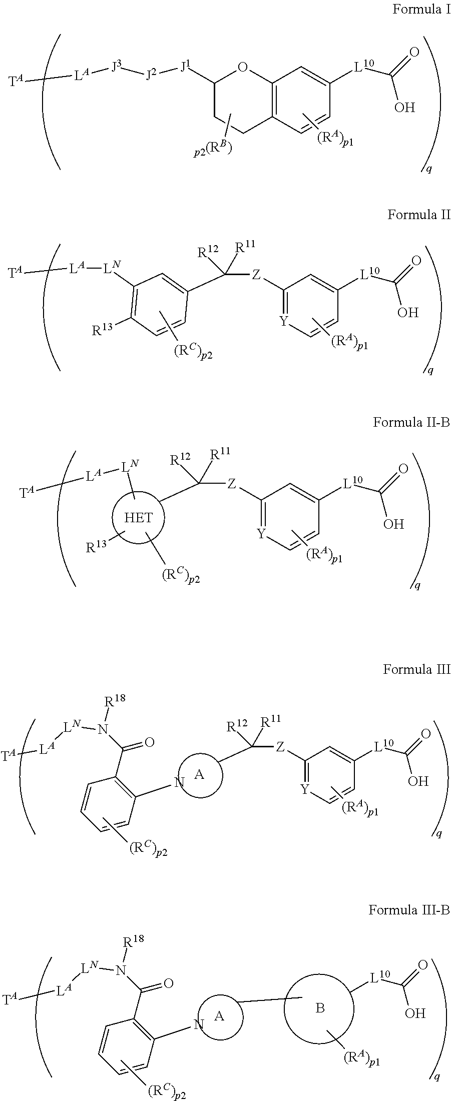

Provided herein are novel compounds (e.g., Formula I, II, II-B, III, or III-B), pharmaceutical compositions, and methods of using related to GPR40. The compounds herein are typically GPR40 agonists, which can be used for treating a variety of disorders, conditions or diseases such as Type 2 diabetes.

### CROSS-REFERENCE TO RELATED APPLICATIONS

This application claims priority to International Application No: PCT/CN2022/071974, filed Jan. 14, 2022, the content of which is herein incorporated by reference in its entirety.

In various embodiments, the present disclosure generally relates to novel antidiabetic compounds, pharmaceutical compositions, and methods of using the same, such as for treating Type 2 diabetes mellitus.

### BACKGROUND

Type 2 diabetes mellitus is a form of diabetes that is characterized by high blood sugar, insulin resistance, and relative lack of insulin. There are several available treatments for Type 2 diabetes, each of which has its own limitations and potential risks. Pharmacologic treatments for diabetes have largely focused on: (1) hepatic glucose production (biguanides, such as phenformin and metformin), (2) insulin resistance (PPAR agonists, such as rosiglitazone, troglitazone, engliazone, balaglitazone, MCC-555, netoglitazone, T-131, LY-300512, LY-818 and pioglitazone), (3) insulin secretion (sulfonylureas, such as tolbutamide, glipizide and glimipiride); (4) incretin hormone mimetics (GLP-1/GIP derivatives and analogs, such as exenatide, liraglutide, dulaglutide, semaglutide, lixisenatide, albiglutide, taspoglutide, and tirzepatide); (5) inhibitors of incretin hormone degradation (DPP-4 inhibitors, such as sitagliptin, alogliptin, vildagliptin, linagliptin, denagliptin and saxagliptin); and (6) SGLT2 inhibitors (canagliflozin, dapagliflozin, empagliflozin, and ertugliflozin).

G-protein-coupled receptor 40 (GPR40) is a cell-surface GPCR that is highly expressed in human (and rodent) islets as well as in insulin-secreting cell lines. The human G-protein-coupled receptor hGPR40 is primarily localized in pancreatic β cells and intestinal enteroendocrine cells. GPR40 is also reported to be expressed in the brain (hippocampus and hypothalamus), hepatocytes, and macrophages. Medium- to long-chain fatty acids (FFAs) are endogenous ligands of GPR40. Upon binding to GPR40, FFAs trigger a signaling cascade that results in increased levels of [Ca²⁺] in β-cells and subsequent stimulation of insulin secretion. In the gut, FFAs also stimulate secretion of incretins, including glucagon-like peptide-1 (GLP-1) and glucose-dependent insulinotropic polypeptide (GIP), cholocystokinine (CCK), and peptide YY (PYY). The recent recognition of the function of GPR40 in modulating insulin secretion has provided insights into regulation of carbohydrate and lipid metabolism in vertebrates, and further provided targets for the development of therapeutic agents for metabolic disorders such as obesity, diabetes, cardiovascular disease and dyslipidemia.

Agonists of G-protein-coupled receptor 40 (GPR40) have been shown to be useful in treating type 2 diabetes mellitus, obesity, hypertension, dyslipidemia, cancer, and metabolic syndrome, as well as cardiovascular diseases, such as myocardial infarction and stroke. New GPR40 agonists that have pharmacokinetic and pharmacodynamic properties suitable for use as human pharmaceuticals are needed.

### BRIEF SUMMARY

Provided herein are compounds, pharmaceutical compositions, and methods of using related to GPR40. The compounds herein are typically GPR40 agonists, which can be used for treating a disorder, condition or disease such as Type 1 or 2 diabetes, obesity, hyperglycemia, glucose intolerance, insulin resistance, hyperinsulinemia, hypercholesterolemia, hypertension, hyperlipoproteinemia, hyperlipidemia, myocardial infarction, stroke, hypertriglylceridemia, dyslipidemia, metabolic syndrome, syndrome X, cardiovascular disease, atherosclerosis, kidney disease, diabetic kidney disease, ketoacidosis, thrombotic disorders, nephropathy, diabetic neuropathy, diabetic retinopathy, sexual dysfunction, dermatopathy, dyspepsia, hypoglycemia, cancer, edema, nonalcoholic steatohepatitis (NASH), lipodystrophy, Prader Willi syndrome, inflammatory bowel diseases including Crohn's disease and ulcerative colitis, irritable bowel syndrome, short bowel syndrome, lymphocytic colitis, rare microscopic colitis, and/or neurodegenerative diseases including but not limited to Alzheimer's disease, Parkinson's disease, amyotrophic lateral sclerosis.

Some embodiments of the present disclosure are directed to compounds of Formula I, or pharmaceutically acceptable salts or esters thereof:

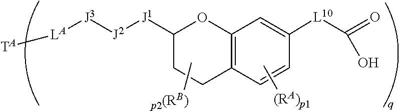

wherein the variables are defined herein. In some embodiments, the compounds of Formula I can have a subformula according to Formula I-1, I-2, 1-3, I-4, I-5, I-6, I-7, 1-8, 1-9, I-1-A, I-4-A, I-4-B, I-4-C, I-5-A, I-5-B, I-5-C, I-1-A-1, I-1-A-2, I-1-A-3, I-1-A-4, I-1-A-5, I-1-A-6, I-1-A-7, I-1-A-8, I-1-A-9, or I-1-A-10, as defined herein.

Some embodiments of the present disclosure are directed to compounds of Formula II, or pharmaceutically acceptable salts or esters thereof:

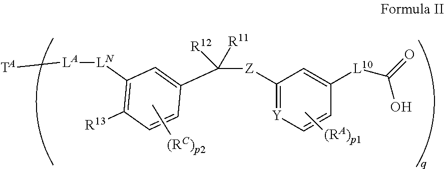

wherein the variables are defined herein. In some embodiments, the compounds of Formula II can have a subformula according to Formula II-1, II-2, II-3, II-4, II-5, II-6, II-7, II-8, II-9, II-1-A, II-4-A, II-5-A, or II-1-A-1, as defined herein.

Some embodiments of the present disclosure are directed to compounds of Formula II-B, or pharmaceutically acceptable salts or esters thereof:

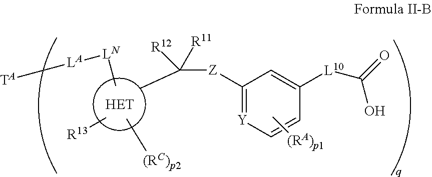

wherein the variables are defined herein. In some embodiments, the compounds of Formula II-B can have a subformula according to Formula II-B-1, II-B-2, or II-B-3, as defined herein.

In some embodiments, the present disclosure provides a compound of Formula III, or a pharmaceutically acceptable salt or ester thereof:

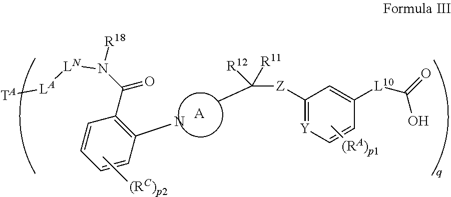

wherein the variables are defined herein. In some embodiments, the compounds of Formula III can have a subformula according to Formula III-1, III-2, III-3, III-4, III-5, III-6, III-7, III-8, III-9, III-1-A, III-4-A, III-5-A, or III-1-A-1, as defined herein.

In some embodiments, the present disclosure provides a compound of Formula III-B, or a pharmaceutically acceptable salt or ester thereof:

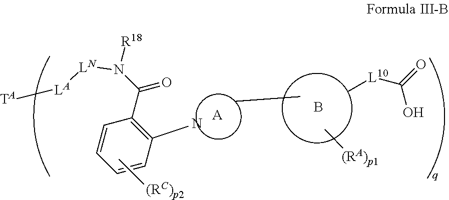

wherein the variables are defined herein. In some embodiments, the compound of Formula III-B can have a subformula according to Formula III-B-1, III-B-2, or III-B-3, as defined herein.

In some embodiments, the present disclosure also provides a compound selected from Table 1 herein, or a pharmaceutically acceptable salt or ester thereof.

In some embodiments, the present disclosure provides a pharmaceutical composition comprising one or more compounds of the present disclosure and optionally a pharmaceutically acceptable excipient. In some embodiments, the pharmaceutical composition comprises a compound of Formula I (e.g., Formula I-1, I-2, I-3, I-4, I-5, I-6, I-7, I-8, I-9, I-1-A, I-4-A, I-4-B, I-4-C, I-5-A, I-5-B, I-5-C, I-1-A-1, I-1-A-2, I-1-A-3, I-1-A-4, I-1-A-5, I-1-A-6, I-1-A-7, I-1-A-8, I-1-A-9, or I-1-A-10), Formula II (e.g., Formula II-1, II-2, II-3, II-4, II-5, II-6, II-7, II-8, II-9, II-1-A, II-4-A, II-5-A, or II-1-A-1), Formula II-B (e.g., Formula II-B-1, II-B-2, or II-B-3), Formula III (e.g., Formula III-1, III-2, III-3, III-4, III-5, III-6, III-7, III-8, III-9, III-1-A, III-4-A, III-5-A, or III-1-A-1), Formula III-B (e.g., III-B-1, III-B-2, or III-B-3), or any of the compounds listed in Table 1 herein, any of the compounds according to Examples 1-221 herein, or a pharmaceutically acceptable salt or ester thereof, and a pharmaceutically acceptable excipient. The pharmaceutical composition can be typically formulated for oral administration. In some embodiments, the pharmaceutical composition is administered to a subject in need to deliver an effective amount of GPR40 agonist in the gastrointestinal tract with minimal or no absorption of GPR40 agonist in systemic circulation.

In some embodiments, the present disclosure provides a method of treating or preventing a disorder, condition or disease that may be responsive to the activation of the GPR40 in a subject in need thereof. In some embodiments, the method comprises administering to the subject an effective amount of one or more compounds of the present disclosure or the pharmaceutical composition herein. In some embodiments, the method comprises administering to the subject an effective amount of a compound of Formula I (e.g., Formula I-1, I-2, I-3, I-4, I-5, I-6, I-7, 1-8, I-9, I-1-A, I-4-A, I-4-B, I-4-C, I-5-A, I-5-B, I-5-C, I-1-A-1, I-1-A-2, I-1-A-3, I-1-A-4, I-1-A-5, I-1-A-6, I-1-A-7, I-1-A-8, I-1-A-9, or I-1-A-10), Formula II (e.g., Formula II-1, II-2, II-3, II-4, II-5, II-6, II-7, II-8, II-9, II-1-A, II-4-A, II-5-A, or II-1-A-1), Formula II-B (e.g., Formula II-B-1, II-B-2, or II-B-3), Formula III (e.g., Formula III-1, III-2, III-3, III-4, III-5, III-6, III-7, III-8, III-9, III-1-A, III-4-A, III-5-A, or III-1-A-1), Formula III-B (e.g., III-B-1, III-B-2, or III-B-3), or any of the compounds listed in Table 1 herein, any of the compounds according to Examples 1-221 herein, or a pharmaceutically acceptable salt or ester thereof, or a pharmaceutical composition comprising the same. In some embodiments, the administering is an oral administration.

In some embodiments, the present disclosure provides a method of treating type 2 diabetes mellitus in a subject in need thereof. In some embodiments, the method comprises administering to the subject a therapeutically effective amount of one or more compounds of the present disclosure or the pharmaceutical composition herein. In some embodiments, the method comprises administering to the subject a therapeutically effective amount of a compound of Formula I (e.g., Formula I-1, I-2, I-3, I-4, I-5, I-6, I-7, I-8, I-9, I-1-A, I-4-A, I-4-B, I-4-C, I-5-A, I-5-B, I-5-C, I-1-A-1, I-1-A-2, I-1-A-3, I-1-A-4, I-1-A-5, I-1-A-6, I-1-A-7, I-1-A-8, I-1-A-9, or I-1-A-10), Formula II (e.g., Formula II-1, II-2, II-3, II-4, II-5, II-6, II-7, II-8, II-9, II-1-A, II-4-A, II-5-A, or II-1-A-1), Formula II-B (e.g., Formula II-B-1, II-B-2, or II-B-3), Formula III (e.g., Formula III-1, III-2, III-3, III-4, III-5, III-6, III-7, III-8, III-9, III-1-A, III-4-A, III-5-A, or III-1-A-1), Formula III-B (e.g., III-B-1, III-B-2, or III-B-3), or any of the compounds listed in Table 1 herein, any of the compounds according to Examples 1-221 herein, or a pharmaceutically acceptable salt or ester thereof, or a pharmaceutical composition comprising the same. In some embodiments, the administering is an oral administration.

In some embodiments, the method herein further comprises administering to the subject an additional therapeutic agent. In some embodiments, the additional therapeutic agent can be PPAR gamma agonists and partial agonists; biguanides; protein tyrosine phosphatase-1B (PTP-1B) inhibitors; dipeptidyl peptidase IV (DPP-IV) inhibitors; insulin or an insulin mimetic; sulfonylureas; a-glucosidase inhibitors; agents which improve a patient's lipid profile, said agents being selected from the group consisting of (i) HMG-COA reductase inhibitors, (ii) bile acid sequestrants, (iii) nicotinyl alcohol, nicotinic acid or a salt thereof, (iv) PPARa agonists, (v) cholesterol absorption inhibitors, (vi) acyl CoA: cholesterol acyltransferase (ACAT) inhibitors, (vii) CETP inhibitors, (viii) PCSK9 inhibitor or antibodies; (ix) apolipoproteins inhibitors; (x) phenolic anti-oxidants; PPARα/γ dual agonists; PPARδ agonists; PPAR α/δ partial agonists; antiobesity compounds; ileal bile acid transporter inhibitors; anti-inflammatory agents; glucagon receptor antagonists; glucokinase activators; GLP-1 and GLP-1 analogs; GLP-1 receptor agonists (peptide and small-molecule); GLP-1/GIP receptor dual agonists; GLP-1/glucagon receptor dual agonists; GLP-1/GIP/insulin receptor triple agonists; GLP-1/GIP/glucagon receptor triple agonists; GIP receptor antibody; GLP-1 analog/GIP receptor antibody; PYY analog; amylin analogs; GPR119 agonist; TGR5 agonist; SSTR3 and/or SSTR5 antagonist or inverse agonist; THRβ agonists; HSD-1 inhibitors; HSD-17 inhibitors and degraders; PNPLA3 inhibitors and degraders; SGLT-2 inhibitors; SGLT-1/SGLT-2 inhibitors; enteric alpha-glucosidase inhibitors; FXR agonists; DGAT1 and/or DGAT2 inhibitors; FGF19 and analogs; FGF21 and analogs; GDF15 and analogs; ANGPTL3 antibody or inhibitor; ANGPTL3/8 antibody; ANGPTL4 inhibitor; Oxyntomodulin; (xi) anti-amyloid beta antibody; (xii) anti-inflammatory agents including but not limited to PDE4 inhibitors, JAK inhibitors, TYK2 inhibitors, S1P receptor modulators, NLRP3 inhibitors, BTK inhibitors, IRAK1 inhibitors, IRAK4 inhibitors, glucocorticoids, anti-TNFα antibodies, anti-IL-12/IL-23 antibodies, (xiii) anti-integrin antibodies including anti-α4β7, anti-α4, anti-β7, anti-MAdCAM-1.

It is to be understood that both the foregoing summary and the following detailed description are exemplary and explanatory only, and are not restrictive of the invention herein.

### DETAILED DESCRIPTION

In various embodiments, the present disclosure provides compounds that are useful for modulating GPR40. The compounds herein typically have no or reduced systemic exposure and therefore are expected to have reduced side effects due to such systemic exposure. In some embodiments, the present disclosure also provides pharmaceutical compositions comprising the compound(s) and methods of using the same, such as in treating type 2 diabetes.

### Compounds

International Application No. PCT/CN2021/109496, filed Jul. 30, 2021, the content of which is incorporated herein by reference in its entirety, describes various conjugates of a GPR40 agonist covalently linked to a carrier. As described in the '496 application and without wishing to be bound by theories, it is believed that when administered, conjugates of GPR40 agonist can have advantages such as modulating GPR40 without side effects or with reduced side effects due to reduced systemic exposure.

The present disclosure is based in part on the discovery that certain GPR40 agonists, if covalently linked to a polar group through a linker with sufficient chain length, the resulted compounds, such as those of Formula I, II, II-B, III, or III-B, as described herein, can be highly potent GPR40 agonists, with an EC50 value less than 50 nM, less than 10 nM, or below 1 nM, when tested according to the Biological Example 1 herein.

In a broad aspect, compounds described herein (e.g., Formula I, II, II-B, III, or III-B) can be viewed as having one or more GPR40 agonist(s) covalently linked to a hydrophilic group through a linker. Typically, compounds described herein have one GPR40 agonist(s) covalently linked to a hydrophilic group through a linker: (GPR40 agonist)-Linker-Hydrophilic group. In some more specific embodiments, the compounds may be typically viewed as connecting the residue of GPR40 agonist of

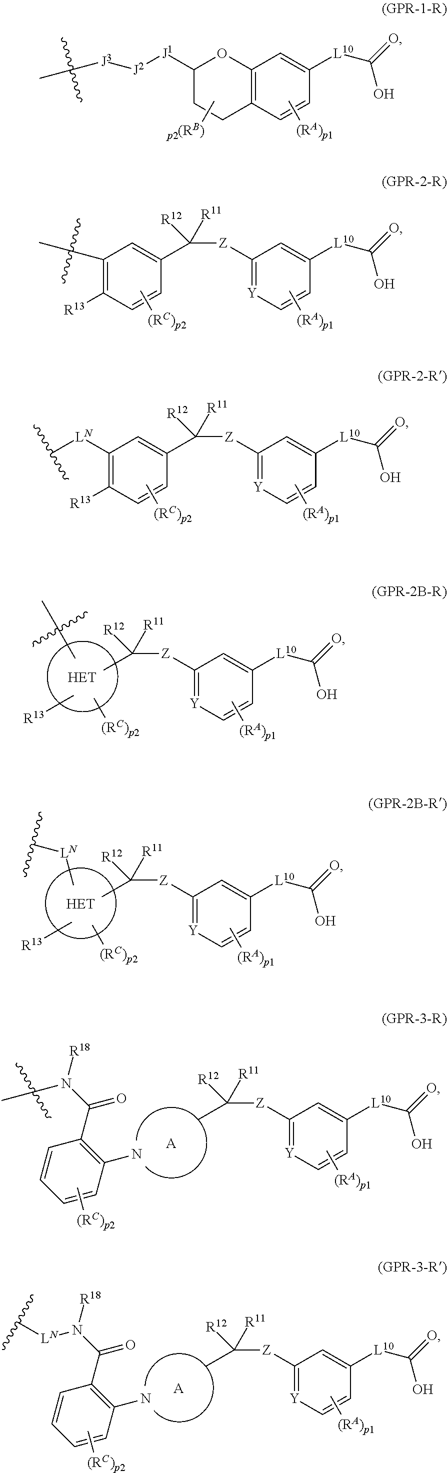

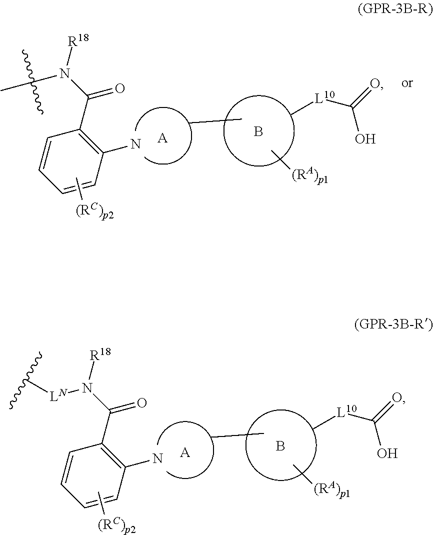

with a hydrophilic group T^{A} through a linker L^{A}, wherein the variables include any of those described and preferred herein in any combinations. To be clear, the dissection of the compounds as residue of GPR40 agonist, linker, and hydrophilic group is merely for convenience of discussions herein, not to limit the compounds herein in any way. For example, for the same compound, there may be different ways to attribute certain structural fragments to be part of the residue of the GPR40 agonist, L^{A}, or T^{A}. For the purposes herein, in such situations, if under one of the ways of attribution, all of the residue of the GPR40 agonist, L^{A}, and T^{A} of the compound are within a respective definition of a genus of compounds herein, then the compound can be said to be within the scope of that genus. Typically, the variables in GPR-1-R, GPR-2-R, GPR-2-R′, GPR-2B-R, GPR-2B-R′, GPR-3-R, GPR-3-R′, GPR-3B-R, or GPR-3B-R′ are such that at least one of the corresponding compounds according to Formula GPR-1, GPR-2, GPR-2B, GPR-3, or GPR-3B is a GPR40 agonist, preferably, having an EC50 of less than 100 nM as measured according to Biological Example 1 herein:

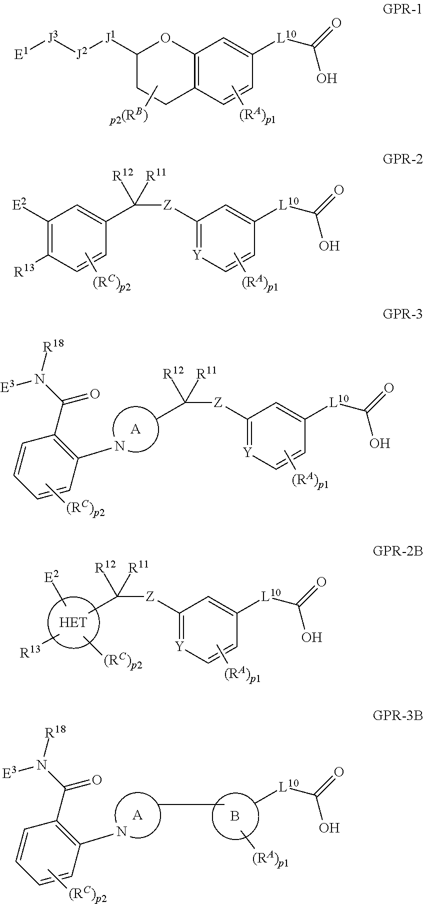

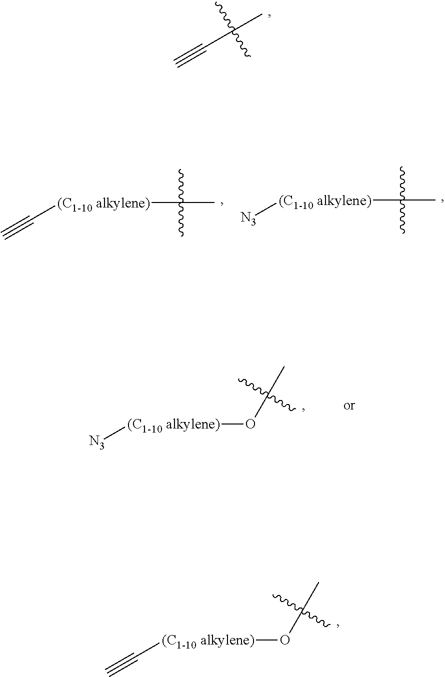

wherein E² is E^{2A} or L^{N}-E^{2A}, wherein E¹ or E^{2A} is hydrogen, C₁₋₄ alkyl, N3,

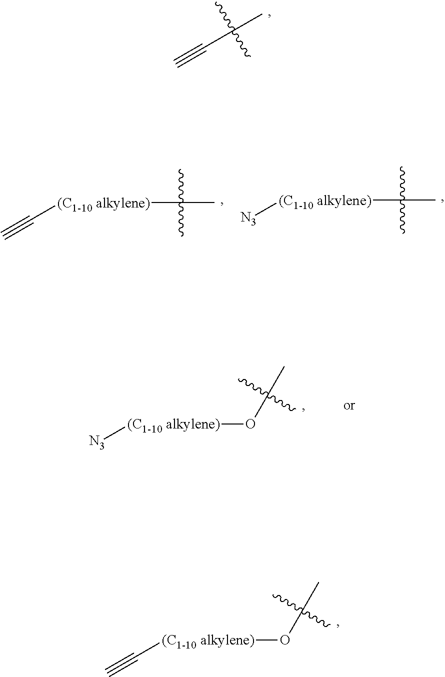

wherein E³ is E^{3A} or L^{N}-E^{3A}, wherein E^{3A} is hydrogen, C₁₋₄ alkyl, N3, and L^{N} is defined herein (such as null or a C₁₋₆ alkylene). In preferred embodiments, the compounds herein with the residue GPR-1-R, GPR-2-R, GPR-2B-R, GPR-3-R, or GPR-3B-R covalently linked to the hydrophilic group T^{A} have a similar EC50 (e.g., within 3-fold) or lower EC50 value compared to at least one (preferably all) of the corresponding compounds of Formula GPR-1, GPR-2, GPR-2B, GPR-3, or GPR-3B. Using Formula I-1 or II-1 as described herein below as illustration, in preferred embodiments, the replacement of L^{A}-T^{A} in Formula I-1 or II-1 with N3 or leads to a comparable or less active GPR40 agonist. In other words, in such embodiments, the compound of Formula I-1 or II-1 has an EC50 comparable or less than that of the corresponding compound of Formula GPR-1 or GPR-2 wherein E¹ or E² is N3 or

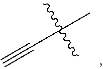 [SMILES: *C#C]

wherein the EC50 is measured according to Biological Example 1 herein.

### Formula I

In some embodiments, the present disclosure provides a compound of Formula I, or a pharmaceutically acceptable salt or ester thereof:

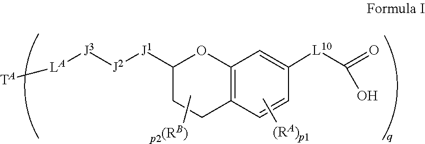

wherein:
L¹⁰ is an alkyelene (e.g., a C₁₋₆ alkylene), optionally substituted with 1-3 substituents independently selected from halogen, optionally substituted C₁₋₆ alkyl, optionally substituted C₂₋₆ alkenyl, optionally substituted C₂₋₆ alkynyl, optionally substituted C₁₋₆ heteroalkyl, optionally substituted C₃₋₆ cycloalkyl, optionally substituted C₁₋₆ alkoxy, optionally substituted C₃₋₆ cycloalkoxy, optionally substituted heterocyclyl, optionally substituted aryl, and optionally substituted heteroaryl, or two substituents are joined to form an optionally substituted ring structure;
R^{A} at each occurrence is independently halogen, CN, optionally substituted C₁₋₆ alkyl, optionally substituted C₃₋₆ cycloalkyl, optionally substituted C₁₋₆ alkoxy, or optionally substituted C₃₋₆ cycloalkoxy, or two R^{A} are joined to form an optionally substituted ring structure; p1 is 0, 1, or 2;
R^{B} at each occurrence is independently halogen, hydroxyl, amino, substituted amino, optionally substituted C₁₋₆ alkyl, optionally substituted C₃₋₆ cycloalkyl, optionally substituted C₁₋₆ alkoxy, or optionally substituted C₃₋₆ cycloalkoxy, or two R^{B} are joined to form an optionally substituted ring structure; p2 is 0, 1, 2, 3, or 4;
J¹ is a bond, an optionally substituted aryl or heteroaryl ring, —C₁₋₆alkylene-N(R¹⁰⁰)—, 3-14 membered optionally substituted heterocyclylene containing at least one ring nitrogen atom, or —C₁₋₆alkylene-(3-14 membered optionally substituted heterocyclylene containing at least one ring nitrogen atom)-, wherein R¹⁰⁰ is hydrogen, optionally substituted alkyl, or optionally substituted cycloalkyl;
J² is a bond or an alkylene, optionally substituted with 1-3 substituents independently selected from halogen, optionally substituted C₁₋₆ alkyl, optionally substituted C₂₋₆ alkenyl, optionally substituted C₂₋₆ alkynyl, optionally substituted C₁₋₆ heteroalkyl, optionally substituted C₃₋₆ cycloalkyl, optionally substituted C₁₋₆ alkoxy, optionally substituted C₃₋₆ cycloalkoxy, or two substituents are joined to form an optionally substituted ring structure;
J³ is an optionally substituted cycloalkyl, heterocyclyl, aryl or heteroaryl ring, and wherein:
q is an integer of 1-10, preferably, 1 or 2, and
T^{A} is a hydrophilic group and L^{A} is a linker. In preferred embodiments,
T^{A} is a hydrophilic group having a terminal atom(s) selected from N, O, S, or C, which is covalently bonded with a first end atom of L^{A}, wherein (1) when the terminal atom(s) is N of a basic amine group, then the corresponding compound T^{A}-(C(O)—CH₃)_{q} has a cLogP of less than 1, wherein the —C(O)—CH₃ is bonded with the terminal N atom(s); (2) when the terminal atom(s) is C of a C(O) group, then the corresponding compound T^{A}-(OH)_{q} has a cLogP of less than 1, wherein the —OH is bonded with the terminal C atom(s); (3) when the terminal atom(s) is S of a SO2 group, then T^{A}-(OH)_{q} has a cLogP of less than 1, wherein the —OH is bonded with the terminal S atom(s); or (4) when (1)-(3) do not apply, then T^{A}-H_{q} has a cLogP of less than 1; and
L^{A} is a linker characterized in that the maximum length between the two end atoms of L^{A} is at least the maximum length between the two end carbon atoms of —(CH₂)₁₀—, wherein (1) when both end atoms of L^{A} are C of a C(O) or S of a SO2 group, then the corresponding compound HO-L^{A}-OH has a cLogP of at least 3, wherein each —OH is bonded with the end C(O) or SO2 group; (2) when only one end atom of L^{A} is C of a C(O) or S of a SO2 group, then the corresponding compound H-L^{A}-OH has a cLogP of at least 3, wherein the —OH is bonded with the end C(O) or SO2 group; or (3) when neither (1) and (2) applies, then the corresponding compound H-L^{A}-H has a cLogP of at least 3.
The term “end atom(s)”, “terminal atom(s)”, and the alike as used herein in connection with a structure, such as L^{A} or T^{A} herein for Formula I, II, or III, should be understood as the attaching point (atom) of the structure with the remainder of the molecule, thus by this definition, these end atoms are non-hydrogen atoms. For example, an alkylene chain of —(CH₂)₁₀-should be understood as having two end carbon atoms.

In some embodiments, q in Formula I is 1, and the compound of Formula I can be characterized as having a Formula I-1:

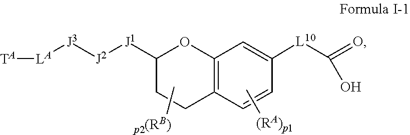

wherein the variables T^{A}, L^{A}, L¹⁰, J¹, J², J³, R^{A}, R^{B}, p1, and p2 include any of those described and preferred herein in any combinations, i.e., the definition and preferred definition of each of the variables can be any of those described herein for the respective variable, which can be combined with a definition (e.g., a preferred definition) of any one or more other variables herein in any and all possible ways.

### Linker L^{A}

As shown herein, the inventors found that several factors are important for the compound of Formula I to be a potent GPR40 agonist. One factor that can determine whether the compound of Formula I can be a potent GPR40 agonist is the length of the linker (e.g., L^{A}) but not the exact chemical structure of the linker. As shown in the Examples section herein, when the length of the linker is below certain threshold, the EC50 value can increase significantly. Accordingly, the present inventors envision that the maximum length between the two end atoms of linker L^{A} in Formula I should be at least that between the two end carbon atoms of an alkyelene chain —(CH₂)₁₀—. In other words, the longest chain length of L^{A} should be equal to or greater than the longest chain length of the alkyelene chain-(CH₂)₁₀—. To further explain, the linker L^{A} can be viewed as having the following structure with two end atoms:

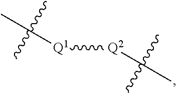

wherein Q¹ represents a non-hydrogen atom of the first end of L^{A} that is bonded with an terminal atom in T^{A}, and Q² represents a non-hydrogen atom of the second end of L^{A} that is bonded with an attaching point in J³, and Q¹ and Q² are connected through a chain or ring/chain structure. Thus, the maximum distance between the two end atoms of linker L^{A} should be understood as the maximum distance between the two connecting points Q¹ and Q² in L^{A}, which under the definition above, should be equal to or greater than the maximum length between the two end carbons of the alkyelene chain-(CH₂)₁₀—. In other words, the maximum distance between the two connecting points Q¹ and Q² in L^{A} should be equal to or greater than that when L^{A} is —(CH₂)₁₀—. Unless otherwise specified or obvious contrary from context, the alkyelene chain such as —(CH₂)₁₀— and the alike herein is not in a cyclic structure. The maximum length between the two end carbons of the —(CH₂)₁₀— can be estimated by computer modeling, measuring the distance between the two end carbons when the alkyelene chain is fully stretched in one direction. Similarly, the maximum length between Q¹ and Q² in L^{A} can be estimated by computer modeling, measuring the distance between Q¹ and Q² when the chain(s) in L^{A} is fully stretched in one direction. Using this method, it would be apparent that a linear alkylene chain having more than 10 carbons in the chain will have a maximum length longer than that of —(CH₂)₁₀—. Similarly, a linear saturated chain structure having more than 10 non-hydrogen atoms in the chain will also have a maximum length longer than that of —(CH₂)₁₀—. In some embodiments, L^{A} can be characterized in that the maximum length between the two end atoms of L^{A} is at least that between the two end carbon atoms of —(CH₂)₁₂—, preferably, at least that of —(CH₂)₁₄—, more preferably, at least that of —(CH₂)₁₆—. In some embodiments, L^{A} can be characterized in that the maximum length between the two end atoms of L^{A} is between (i) the maximum length between the two end carbon atoms of —(CH₂)₁₂— and (ii) the maximum length between the two end carbon atoms of —(CH₂)₅₀—.

In some embodiments, in Formula I (e.g., Formula I-1), L^{A} can be represented by a formula of (X) m, wherein X at each occurrence is independently CR₂, C(═O), —C(R)═C(R)—,

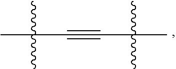 [SMILES: *C#C*]

SiR₂, O, S, SO, SO₂, NR, [NR₂]⁺, or a ring structure, preferably 3-10 membered ring structure, wherein R at each occurrence is independently hydrogen, halogen, optionally substituted C₁₋₄ alkyl, or optionally substituted C₁₋₄ alkoxy, typically hydrogen or C₁₋₄ alkyl, and the integer m is at least 10, such as at least 12, at least 14, at least 16, at least 18, at least 20, at least 50, such as 12-50, 16-50, etc. To be clear, the formula (X) m should be understood as a linear structure having each X connected to another two X groups except the two end X groups (the two X groups that are directly connected to T^{A} or J³), i.e., -X-X-X . . . X—, with the total number of X being m. In cases wherein X is a ring structure, preferably 3-10 membered ring structure, it should be understood that the ring structure is attached to two adjacent X units through one or two ring atoms, for example, X can have a structure such as

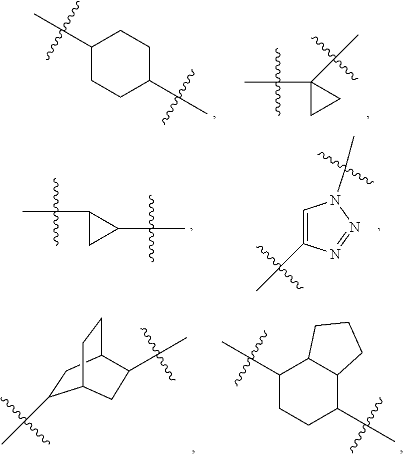 [SMILES: *C1CCC(*)CC1 ; *C1(*)CC1 ; *C1CC1* ; *c1cn(*)nn1 ; *C1CC2CCC1CC2* ; *C1CCC(*)C2CCCC12]

etc. The “ring structure” or “3-10 membered ring structure” and the alike as used herein is not limited to any particular ring system and can include a carbocyclic ring, a heterocyclic ring, an aromatic ring, a heteroaryl ring, or a combination thereof, which can be substituted or unsubstituted. For example, the “3-10 membered ring structure” can be monocyclic, bicyclic, or tricyclic, which can include a fused, spiro, or bridged ring system. For clarity, two ring systems connected through a single bond should be viewed as separate ring system and can each account for one X unit herein. For example, for a structure like

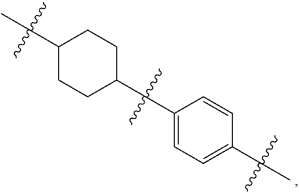

it may be viewed as two X units connected, with one X being a cyclohexylene and the other X being a phenylene. Typically, in L^{A}, 0, 1, or 2 instances of X representing a ring structure, preferably 3-10 membered ring structure (such as a C₃₋₆ cycloalkyl such as cyclopropyl, a 5 or 6 membered heteroaryl, such as a triazole ring). In some embodiments, two consecutive X can be —C(O)O— or —C(O) NR—. In some embodiments, one instance of X can be a cyclopropane, cyclobutane or bicyclobutane [1.1] ring. In some embodiments, one instance of X can be a 5 or 6-membered heteroaryl, such as a triazole ring. In some embodiments, one instance of X can be a cyclopentane, cyclohexane or cycloheptane ring. Typically, one of the end X group connects to T^{A} through a carbon atom. The total number of non-hydrogen atoms of L^{A} can be typically between 10-100, such as 12-30, 14-50, 16-50, 18-100, etc.

Another factor that can determine whether the compound of Formula I can be a potent GPR40 agonist is the hydrophobicity of the linker (e.g., L^{A}) but not the exact chemical structure of the linker. In general, L^{A} should be a hydrophobic moiety. In some embodiments, both end atoms of L^{A} are C of a C(O) or S of a SO2 group, in such embodiments, the hydrophobicity of L^{A} can be typically characterized in that the corresponding compound HO-L^{A}-OH has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc., wherein each of the —OH is bonded with the end C(O) or SO2 group. In some embodiments, only one end atom of L^{A} is C of a C(O) or S of a SO2 group, in such embodiments, the hydrophobicity of L^{A} can be typically characterized in that the corresponding compound H-L^{A}-OH has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc., wherein the —OH is bonded with the end C(O) or SO2 group. In some embodiments, neither of the end atoms of L^{A} is C of a C(O) or S of a SO2 group, in such embodiments, in such embodiments, the hydrophobicity of L^{A} can be characterized in that the corresponding compound H-L^{A}-H has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc.

For example, in some embodiments, L^{A} is (X) m-1—C(O)—, wherein the C(O) end is bonded with T^{A}, wherein X at each occurrence is independently CR₂, C(═O), —C(R)═C(R)—,

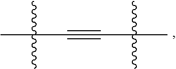 [SMILES: *C#C*]

SiR₂, O, S, SO₂, NR, [NR₂]⁺ or a ring structure, preferably 3-10 membered ring structure, provided that the end X group (i.e., the X group in L^{A} that is furthest away from the C(O) end) is not C(O) or SO2, wherein R at each occurrence is independently hydrogen, halogen, optionally substituted C₁₋₄ alkyl, or optionally substituted C₁₋₄ alkoxy, typically hydrogen or C₁₋₄ alkyl, and the integer m is at least 10, such as at least 12, at least 14, at least 16, at least 18, at least 20, at least 50, such as 12-50, 16-50, etc., and the hydrophobicity of —(X) m-1—C(O)— is characterized in that the corresponding compound H—(X) m-1-COOH should have a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc. The “m-1” should be understood as the integer m minus 1, not to be misunderstood as a different designated variable.

In some embodiments, L^{A} is (X) m, wherein X at each occurrence is independently CR₂, C(═O), —C(R)═C(R)—,

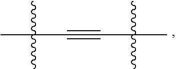 [SMILES: *C#C*]

SiR₂, O, S, SO₂, NR, [NR₂]⁺ or a ring structure, preferably 3-10 membered ring structure, provided that neither of the end X groups is C(O) or SO₂, wherein R at each occurrence is independently hydrogen, halogen, optionally substituted C₁₋₄ alkyl, or optionally substituted C₁₋₄ alkoxy, typically hydrogen or C₁₋₄ alkyl, and the integer m is at least 10, such as at least 12, at least 14, at least 16, at least 18, at least 20, at least 50, such as 12-50, 16-50, etc., then the corresponding compound H—(X)ₘ—H should have a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc.

As would be apparent to those skilled in the art, the term cLogP (or CLogP) refers to calculated LogP. For the purpose of this application, the cLogP value can be obtained using PerkinElmer's ChemDraw Professional software, version 20.0.0.41 or equivalent software using the same calculation method. The following shows exemplary cLogP values of a few compounds using the ChemDraw Professional software above:

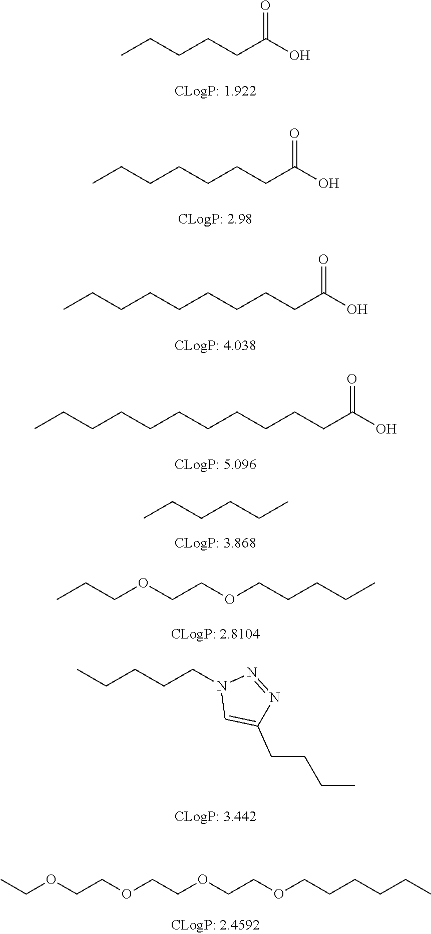

Thus, a compound having a cLogP of at least 3 should be about the same or more hydrophobic than octanoic acid. A compound having a cLogP of at least 4 should be about the same or more hydrophobic than decanoic acid. A compound having a cLogP of at least 5 should be about the same or more hydrophobic than lauric acid.

In some embodiments, L^{A} is —X₁₂₋₃₀-(e.g., —X₁₄—, —X₁₆—, —X₁₈—, —X₂₀—, —X₂₄—, —X₁₄₋₃₀—, —X₁₆₋₃₀—, —X₁₈₋₃₀—, etc.), wherein X at each occurrence is independently CR₂, C(═O), —C(R)—C(R)—,

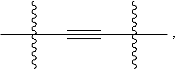 [SMILES: *C#C*]

SiR₂, O, S, SO₂, NR, [NR₂]⁺, or a ring structure, preferably 3-10 membered ring structure, provided that neither of the end X groups is C(O) or SO2; wherein R at each occurrence is independently hydrogen, halogen, optionally substituted C₁₋₄ alkyl, or optionally substituted C₁₋₄ alkoxy, typically hydrogen or C₁₋₄ alkyl, and wherein the hydrophobicity of L^{A} can be characterized in that the corresponding compound H-L^{A}-H has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc. In some embodiments, the total number of non-hydrogen atoms of L^{A} is between 12-100, such as 12, 14, 16, 18, 20, 24, 30, 40, 50, 100, or any ranges between the recited values, such as 12-30, 14-50, 16-50, 18-100, etc. In some embodiments, two consecutive X can represent-C(O)O— or —C(O) NR—. In some embodiments, one or more (e.g., 1 or 2) instances of X can be a ring structure selected from cyclopropane, cyclobutane, bicyclobutane [1.1], cyclopentane, cyclohexane or cycloheptane. In some embodiments, one or more (e.g., 1) instances of X can be a ring structure selected from phenyl or 5 or 6-membered heteroaryl, such as triazole. In some embodiments, R is hydrogen.

In some embodiments, L^{A} is -X12-30—C(O)—, wherein the C(O) is directly bonded with T^{A}, and wherein X at each occurrence is independently CR₂, C(═O), —C(R)═C(R)—,

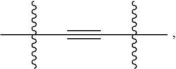 [SMILES: *C#C*]

SiR₂, O, S, SO₂, NR, [NR₂]⁺, or a ring structure, preferably 3-10 membered ring structure, provided that the end X group is not C(O) or SO2; wherein R at each occurrence is independently hydrogen, halogen, optionally substituted C₁₋₄ alkyl, or optionally substituted C₁₋₄ alkoxy, typically hydrogen or C₁₋₄ alkyl, and wherein the hydrophobicity of L^{A} can be characterized in that the corresponding compound H—X₁₂₋₃₀—C(O)—OH has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc. In some embodiments, the total number of non-hydrogen atoms of L^{A} is between 14-100, such as 14, 16, 18, 20, 24, 30, 40, 50, 100, or any ranges between the recited values, such as 14-30, 14-50, 16-50, 18-100, etc. In some embodiments, two consecutive X can be —C(O)O— or —C(O) NR—. In some embodiments, one or more instances (e.g., 1 or 2) of X can be a ring structure selected from cyclopropane, cyclobutane, bicyclobutane [1.1], cyclopentane, cyclohexane or cycloheptane. In some embodiments, one or more instances (e.g., 1) of X can be a ring structure selected from phenyl or 5 or 6-membered heteroaryl, such as triazole. In some embodiments, R is hydrogen.

In some preferred embodiments, L^{A} is —C₁₂₋₃₀ alkylene- or —C₁₂₋₃₀ alkylene-C(O)—, wherein the —C₁₂₋₃₀ alkylene- is optionally substituted, wherein the optional substituents can optionally be joined together to form a double bond, triple bond, or a ring structure (typically a 3-10 membered ring structure), wherein the end carbon atoms of the —C₁₂₋₃₀ alkylene- are not substituted with oxo (═O), wherein the longest chain length of L^{A} is at least that of —(CH₂)₁₂—, such as at least that of —(CH₂)₁₄—, at least that of —(CH₂)₁₆—, at least that of —(CH₂)₁₈—, and wherein the hydrophobicity of L^{A} can be characterized in that the corresponding compound H—C₁₂₋₃₀ alkylene-H or H—C₁₂₋₃₀ alkylene-C(O)—OH has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc. In some embodiments, the total number of non-hydrogen atoms of L^{A} is between 14-100, such as 14, 16, 18, 20, 24, 30, 40, 50, 100, or any ranges between the recited values, such as 14-30, 14-50, 16-50, 18-100, etc. In some embodiments, L^{A} is unsubstituted —C₁₂₋₃₀ alkylene-, such as a linear or branched C₁₂₋₃₀ alkylene. In some embodiments, L^{A} is unsubstituted —C₁₂₋₃₀ alkylene-C(O)—, wherein the C₁₂₋₃₀ alkylene can be linear or branched. In some preferred embodiments, L^{A} is —C₁₂₋₃₀ alkylene- or —C₁₂₋₃₀ alkylene-C(O)—, wherein the C₁₂₋₃₀ alkylene is a linear and unsubstituted —C₁₂₋₃₀ alkylene-.

In some embodiments, L^{A} is a 12-30 membered heteroalkylene or -(12-30 membered heteroalkylene)-C(O)—, wherein the 12-30 membered heteroalkylene is optionally substituted and contains 1-6 heteroatoms independently selected from O, N, and S, wherein the sulfur atom(s), if present, is optionally oxidized, wherein the optional substituents can optionally be joined together to form a double bond, triple bond, or a ring structure (typically a 3-10 membered ring structure), wherein the end atoms of the 12-30 membered heteroalkylene are not C of a C(O) or S of a SO2 group, wherein the longest chain length of L^{A} is at least that of —(CH₂)₁₂—, such as at least that of —(CH₂)₁₄—, at least that of —(CH₂)₁₆—, at least that of —(CH₂)₁₈—, and wherein the hydrophobicity of L^{A} can be characterized in that the corresponding compound H-(12-30 membered heteroalkylene)-H or H-(12-30 membered heteroalkylene)-C(O)—OH has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc. In some embodiments, the total number of non-hydrogen atoms of L^{A} is between 14-100, such as 14, 16, 18, 20, 24, 30, 40, 50, 100, or any ranges between the recited values, such as 14-30, 14-50, 16-50, 18-100, etc. In some embodiments, L^{A} is unsubstituted 12-30 membered heteroalkylene, such as a linear or branched 12-30 membered heteroalkylene. In some embodiments, L^{A} is unsubstituted-(12-30 membered heteroalkylene)-C(O)—, wherein the 12-30 membered heteroalkylene can be linear or branched. In some preferred embodiments, L^{A} is 12-30 membered heteroalkylene or -(12-30 membered heteroalkylene)-C(O)—, wherein the 12-30 membered heteroalkylene is a linear and unsubstituted. In some embodiments, the 12-30 membered heteroalkylene includes 1, 2, 3, 4, or 5 heteroatoms independently selected from O, S, and N.

In some preferred embodiments, L^{A} can be characterized as having a structure according to

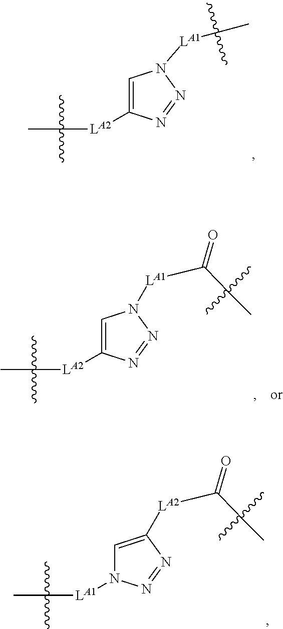

wherein the carbonyl is directly bonded with T^{A}, wherein L^{A1} and L^{A2} are each independently a bond, an optionally substituted —C₁₋₃₀ alkylene-, or an optionally substituted —C₁₋₃₀ heteroalkylene-containing 1-6 heteroatoms independently selected from O, N, and S, wherein the sulfur atom(s), if present, is optionally oxidized, wherein the optional substituents can optionally be joined together to form a double bond, triple bond, or a ring structure, wherein the end atoms of the L^{A1} and L^{A2} that are bonded with T^{A} or J³, as applicable, are not C of a C(O) or S of a SO2 group, wherein the longest chain length of L^{A} is at least that of —(CH₂)₁₂—, such as at least that of (CH₂)₁₄—, at least that of —(CH₂)₁₆, at least that of —(CH₂)₁₈—, and wherein the hydrophobicity of L^{A} can be characterized in that the corresponding compound

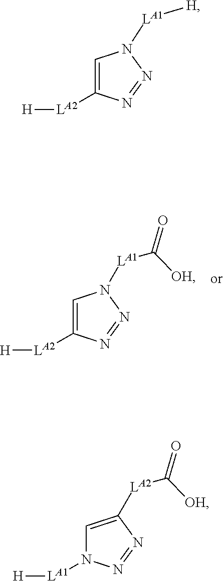

has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc. In some embodiments, the total number of non-hydrogen atoms of L^{A} is between 14-100, such as 14, 16, 18, 20, 24, 30, 40, 50, 100, or any ranges between the recited values, such as 14-30, 14-50, 16-50, 18-100, etc. In some embodiments, L^{A1} is a C₁₋₂₀ alkylene, such as a linear alkylene, (CH₂)₁₋₂₀. In some embodiments, L^{A1} is a C1-10 heteroalkylene having 1-3 oxygen atoms, such as —O—(C₁₋₁₀ alkylene)-, etc. In some embodiments, L^{A2} is a bond. In some embodiments, L^{A2} is a C₁₋₂₀ alkylene, such as a linear alkylene, (CH₂)₁₋₂₀. In some embodiments, L^{A2} is a C₁₋₁₀ heteroalkylene having 1-3 oxygen atoms, such as —O—(C₁₋₁₀ alkylene)-, etc.

In some embodiments, L^{A} can be characterized as having a structure according to

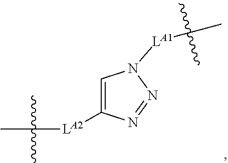

wherein L^{A1} and L^{A2} are each independently a bond, —C₁₋₃₀ alkylene-, or —C₁₋₃₀ heteroalkylene-containing 1-6 heteroatoms independently selected from O, N, and S, wherein the sulfur atom(s), if present, is optionally oxidized, wherein the end atoms of the L^{A1} and L^{A2} that are bonded with T^{A} or J³, as applicable, are not C of a C(O) or S of a SO2 group, wherein the total number of non-hydrogen atoms of L^{A} is between 15-50, such as 18, 20, 25, 30, 35, 40, 45, or 50, or any ranges between the recited value, wherein the longest chain length of L^{A} is at least that of —(CH₂)₁₂—, such as at least that of (CH₂)₁₄—, at least that of —(CH₂)₁₆—, at least that of —(CH₂)₁₈—, wherein the hydrophobicity of L^{A} can be characterized in that the corresponding compound H-L^{A}-H has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc. In some embodiments, L^{A1} is a bond (i.e., not present, the triazole nitrogen atom is directly bonded with J³ or T^{A}). In some embodiments, L^{A2} is a bond. In some embodiments, L^{A1} is a C₁₋₂₀ alkylene, such as a linear alkylene, (CH₂)₁₋₂₀. In some embodiments, L^{A1} is a C₁₋₁₀ heteroalkylene having 1-3 oxygen atoms, such as —O—(C₁₋₁₀ alkylene)-, etc. In some embodiments, L^{A2} is a C₁₋₂₀ alkylene, such as a linear alkylene, (CH₂)₁₋₂₀. In some embodiments, L^{A2} is a C₁₋₁₀ heteroalkylene having 1-3 oxygen atoms, such as —O—(C₁₋₁₀ alkylene)-, etc.

In some embodiments, L^{A} can be characterized as having a structure according to

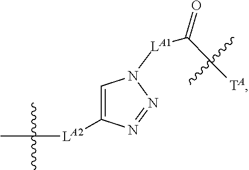

wherein L^{A1} and L^{A2} are each independently a bond, —C₁₋₃₀ alkylene-, or —C₁₋₃₀ heteroalkylene-containing 1-6 heteroatoms independently selected from O, N, and S, wherein the sulfur atom(s), if present, is optionally oxidized, wherein the end atom of the L^{A2} that is bonded with J³ is not C of a C(O) or S of a SO2 group, wherein the total number of non-hydrogen atoms of L^{A} is between 15-50, such as 18, 20, 25, 30, 35, 40, 45, or 50, or any ranges between the recited value, wherein the longest chain length of L^{A} is at least that of —(CH₂)₁₂—, such as at least that of —(CH₂)₁₄—, at least that of —(CH₂)₁₆—, at least that of —(CH₂)₁₈—, wherein the hydrophobicity of L^{A} can be characterized in that the corresponding compound

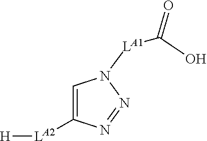

has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc. In some embodiments, L^{A1} is a bond. In some embodiments, L^{A2} is a bond. In some embodiments, L^{A1} is a C₁₋₂₀ alkylene, such as a linear alkylene, (CH₂)₁₋₂₀. In some embodiments, L^{A1} is a C₁₋₁₀ heteroalkylene having 1-3 oxygen atoms, such as —O—(C₁₋₁₀ alkylene)-, etc. In some embodiments, L^{A2} is a C₁₋₂₀ alkylene, such as a linear alkylene, (CH₂)₁₋₂₀. In some embodiments, L^{A2} is a C₁₋₁₀ heteroalkylene having 1-3 oxygen atoms, such as —O—(C₁₋₁₀ alkylene)-, etc.

In some embodiments, L^{A} can be characterized as having a structure according to

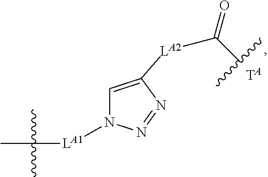

wherein L^{A1} and L^{A2} are each independently a bond, C₁₋₃₀ alkylene-, or —C₁₋₃₀ heteroalkylene-containing 1-6 heteroatoms independently selected from O, N, and S, wherein the sulfur atom(s), if present, is optionally oxidized, wherein the end atom of the L^{A1} that is bonded with J³ is not C of a C(O) or S of a SO2 group, wherein the total number of non-hydrogen atoms of L^{A} is between 15-50, such as 18, 20, 25, 30, 35, 40, 45, or 50, or any ranges between the recited value, wherein the longest chain length of L^{A} is at least that of (CH₂)₁₂, such as at least that of —(CH₂)₁₄—, at least that of —(CH₂)₁₆—, at least that of —(CH₂)₁₈—, wherein the hydrophobicity of L^{A} can be characterized in that the corresponding compound

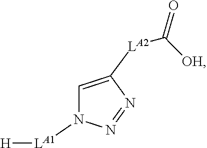

has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc. In some embodiments, L^{A1} is a bond. In some embodiments, L^{A2} is a bond. In some embodiments, L^{A1} is a C₁₋₂₀ alkylene, such as a linear alkylene, (CH₂)₁₋₂₀. In some embodiments, L^{A1} is a C₁₋₁₀ heteroalkylene having 1-3 oxygen atoms, such as —O—(C₁₋₁₀ alkylene)-, etc. In some embodiments, L^{A2} is a C₁₋₂₀ alkylene, such as a linear alkylene, (CH₂)₁₋₂₀. In some embodiments, L^{A2} is a C₁₋₁₀ heteroalkylene having 1-3 oxygen atoms, such as —O—(C₁₋₁₀ alkylene)-, etc.

### Hydrophilic Group T^{A}

Another factor that can determine whether the compound of Formula I can be a potent GPR40 agonist is the hydrophilicity or polarity of T^{A}, but the exact chemical structure of T^{A} is not as important. In Formula I, T^{A} is a hydrophilic group having a terminal atom(s) selected from N, O, S, or C, which is covalently bonded with an end atom such as carbon or nitrogen atom(s) of L^{A}, wherein (1) when the terminal atom(s) is N of a basic amine group, then the corresponding compound T^{A}-(C(O)—CH₃)_{q} has a cLogP of less than 1, wherein the —C(O)—CH₃ is bonded with the terminal N atom(s); (2) when the terminal atom(s) is C of a C(O) group, then the corresponding compound T^{A}-(OH)_{q} has a cLogP of less than 1, wherein the -OH is bonded with the terminal C atom(s); (3) when the terminal atom(s) is S of a SO₂ group, then the corresponding compound T^{A}-(OH), has a cLogP of less than 1, wherein the —OH is bonded with the terminal S atom(s); or (4) when (1)-(3) do not apply, then T^{A}-H_{q} has a cLogP of less than 1. In some embodiments, the terminal atom(s) is N of a basic amine group, and T^{A} is characterized in that the corresponding compound T^{A}-(C(O)—CH₃)_{q} has a cLogP of less than 0 (e.g., less than −1, less than −2, less than −3, less than −3.5, less than −4, or even lower). As used herein, a basic amine group refers to any amine group that has an aqueous pKa at least 5 (based on its conjugate acid at room temperature). In other words, in embodiments where the terminal atom(s) of T^{A} is N of a basic primary or secondary amine group, [T^{A}-H₂]⁺has an aqueous pKa of 5 or above with respect to the terminal nitrogen atom at issue. In some embodiments, the terminal atom(s) is C of a C(O) group, and T^{A} is characterized in that the corresponding compound T^{A}-(OH), has a cLogP of less than 0 (e.g., less than −1, less than −2, less than −3, less than −3.5, less than −4, or even lower). In some embodiments, the terminal atom(s) is S in a SO₂ group, and T^{A} is characterized in that the corresponding compound T^{A}-(OH)_{q} has a cLogP of less than 0 (e.g., less than −1, less than −2, less than −3, less than −3.5, less than −4, or even lower). In some embodiments, the terminal atom(s) is not N of a basic amine group, C of a C(O) group, or S in a SO₂ group, and T^{A} is characterized in that the corresponding compound T^{A}-H_{q} has a cLogP of less than 0 (e.g., less than −1, less than −2, less than −3, less than −3.5, less than −4, or even lower).

In some preferred embodiments, T^{A} can contain a charged group, including positively charged, such as containing a quaternary nitrogen atom, negatively charged, such as SO₃⁻, or containing a zwitterion structure. When T^{A} contains a charged group, it should be understood that a counterion, preferably, a pharmaceutically acceptable anion or cation, if necessary, exists to balance the charges so that the compound of Formula I is overall neutral. Pharmaceutically acceptable anions are known in the art, which are typically derived from a pharmaceutically acceptable acid, e.g., Cl⁻, etc. Pharmaceutically acceptable cations are also known in the art, such as alkali cations such as Na⁺, etc.

In some embodiments, T^{A} is characterized as having a charged group (including zwitterion structures) or a group that can become charged at pH 7, such as primary amine, secondary amine, tertiary amine, quaternary amine, carboxylic acid, etc.

In some embodiments, T^{A} is characterized as having at least two hydrogen bond donors.

In some embodiments, T^{A} is characterized as having at least two hydrogen bond acceptors.

In some embodiments, T^{A} is T^{A1}, T^{A1}-L^{B}-, T^{A1}-L^{B}-(heteroalkylene), or T^{A1}-L^{B}-(heteroalkylene)-L^{B}-, wherein L^{B} at each occurrence is independently —SO₂—, —C(═O)—, or a moiety selected from:

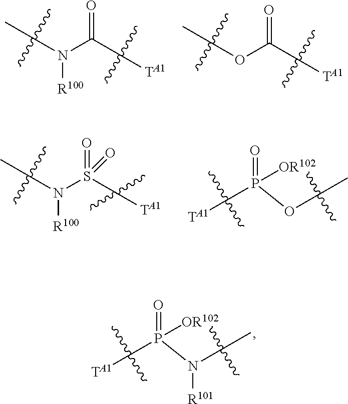

wherein R¹⁰⁰, R¹⁰¹ and R¹⁰² at each occurrence is independently hydrogen, optionally substituted alkyl, or optionally substituted cycloalkyl, wherein T^{A1} is characterized as having a structure of:

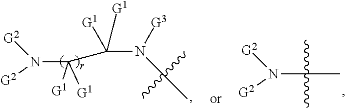

wherein G¹ at each occurrence is independently hydrogen, halogen, optionally substituted C₁₋₄ alkyl, or optionally substituted C₁₋₄ heteroalkyl (such as optionally substituted C₁₋₄ alkoxy), or two gem G¹ group can join to form an ═O, ═NH, or ═N(C₁₋₄ alkyl), or two or more G¹ groups can join to form a ring structure, the integer r is 1-10, such as 1 or 2,
G² at each occurrence is independently hydrogen, G¹⁰, C(O)-G¹⁰, SO₂G¹⁰, C(O)—NH-G¹⁰, or SO₂NHG¹⁰, C(O)—O-G¹⁰, or SO₂OG¹⁰, wherein G¹⁰ at each occurrence is independently an optionally substituted alkyl, an optionally substituted heteroalkyl, or an optionally substituted 3-10 membered ring structure, which can be carbocyclic, heterocyclic, aromatic, heteroaromatic, or a combination thereof,
G³ is hydrogen or an optionally substituted alkyl (e.g., C₁₋₄ alkyl);
or two G² or one G² and one G¹ or one G¹ and G³ can be joined to form a ring structure such as a 3-10 membered ring structure,
wherein the fragment

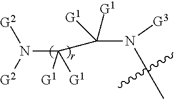

is further characterized in that (i) when the CG¹G¹ group next to the NG³ is not C(═O), the corresponding compound

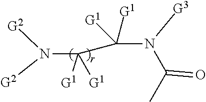

has a cLogP less than 1, preferably, less than 0 (e.g., less than −1, less than −2, less than −3, less than −3.5, less than −4, or even lower), or (ii) when the CG¹G¹ group next to the NG³ is C(═O), the corresponding compound

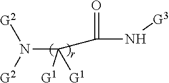

has a cLogP less than 1, preferably, less than 0 (e.g., less than −1, less than −2, less than −3, less than −3.5, less than −4, or even lower), and
wherein the fragment

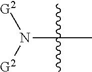

is further characterized in that (i) when the nitrogen is a basic nitrogen, the corresponding compound (G²)₂N—C(O)—CH₃ has a cLogP less than 1, preferably, less than 0 (e.g., less than −1, less than −2, less than −3, less than −3.5, less than −4, or even lower), or (ii) when the nitrogen is a nonbasic nitrogen, the corresponding compound (G²)₂N—H has a cLogP less than 1, preferably, less than 0 (e.g., less than −1, less than −2, less than −3, less than −3.5, less than −4, or even lower). In preferred embodiments, G³ is hydrogen. In some preferred embodiments, T^{A} is T^{A1} or T^{A1}-L^{B}-. In some embodiments, T^{A} is T^{A1}-L^{B}-(heteroalkylene) or T^{A1}-L^{B}-(heteroalkylene)-L^{B}-, wherein the heteroalkylene can be a glycol chain, such as a polyethylene glycol chain.

In some embodiments, T^{A} is T^{A1}. In some embodiments, T^{A} is T^{A1}—C(═O)—. In some embodiments, T^{A1} is characterized as having a structure of:

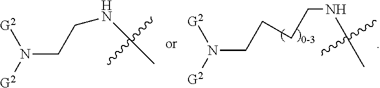

In some embodiments, T^{A1} is characterized as having a structure of:

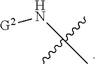

In some embodiments, G² at each occurrence independently can have a structure of

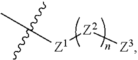

wherein n is an integer of 0-100, preferably, 0-10 (e.g., 0-4, 1-5 or 2-6 etc.), Z¹ is CH₂, C(O), SO, SO₂, CH(C₁₋₄ alkyl), C(C₁₋₄ alkyl)(C₁₋₄ alkyl) or 3-10 membered ring structure, Z² at each occurrence is independently CH₂, O, S, SO, SO₂, C(O), CH(C₁₋₄ alkyl), C(C₁₋₄ alkyl)(C₁₋₄ alkyl), NH, N(C₁₋₄ alkyl), [N(C₁₋₄ alkyl)(C₁₋₄ alkyl)]⁺ or 3-10 membered ring structure, and Z³ is H, C₁-4 alkyl, OH, SO₃H, COOH, NH₂, NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, SO₂NH₂, CONH(C₁₋₄ alkyl), CON(C₁₋₄ alkyl)(C₁₋₄ alkyl), SO₂NH(C₁₋₄ alkyl), or SO₂N(C₁₋₄ alkyl)(C₁₋₄ alkyl), wherein each of the C₁₋₄ alkyl is independently selected and optionally substituted, for example, with 1-3 G²⁰ groups defined herein. In some embodiments, none of the Z² is a 3-10 membered ring structure. In some embodiments, one or more instances of Z² is a 3-10 membered ring structure, such as a nitrogen containing ring, for example,

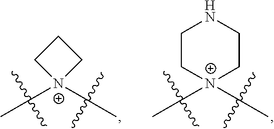 [SMILES: *[N+]1(*)CCC1 ; *[N+]1(*)CCNCC1]

etc. In some embodiments, the Z² directly connects to Z³ is a 3-10 membered ring structure, such as a nitrogen containing ring, for example,

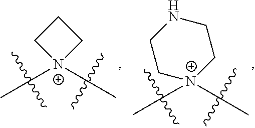 [SMILES: *[N+]1(*)CCC1 ; *[N+]1(*)CCNCC1]

etc. Preferably, at most 2 instances of Z² is a 3-10 membered ring structure. In some embodiments, G² at each occurrence is independently hydrogen, an optionally substituted C₁₋₆ alkyl or an optionally substituted C₁₋₆ heteroalkyl, for example, when substituted, the C₁₋₆ alkyl can be substituted with 1-3 substituents independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), and SO₂N(C₁₋₄ alkyl)(C1-4 alkyl), wherein each of the C₁₋₄ alkyl is independently selected and optionally substituted, for example, with 1-3 G²⁰ groups defined herein. The G²⁰ group herein at each occurrence can be independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), SO₂N(C₁₋₄ alkyl)(C₁₋₄ alkyl), or 3-10 membered ring structure, wherein each of the C₁₋₄ alkyl is independently selected and optionally substituted, for example, with 1-3 G²¹ groups defined herein. The G²¹ group herein at each occurrence can be independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), SO₂N(C₁₋₄ alkyl)(C₁₋₄ alkyl) or 3-10 membered ring structure. In some embodiments, one or more instances of G² is C₁-4 alkyl, optionally substituted with COOH or an amide derivative, such as CONH—(C₁₋₄ alkylene)-NH₂ or CONH—(C₁₋₄ alkylene)-[N(C₁₋₄ alkyl)(C₁₋₄ alkyl)(C₁₋₄ alkyl)]⁺. For example, in some embodiments, one or two instances of G² is —(C₁₋₄ alkylene)-COOH, such as —CH₂COOH, and any remaining instance(s) of G² is C₁-4 alkyl. In some embodiments, one or two instances of G² is —(C₁₋₄ alkylene)-CONH—(C₁₋₄ alkylene)-NH₂, such as

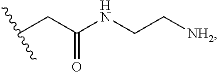 [SMILES: *CC(=O)NCCN]

and any remaining instance(s) of G² is C₁₋₄ alkyl. In some embodiments, one or two instances of G² is —(C₁₋₄ alkylene)-CONH—(C₁₋₄ alkylene)-[N(C₁₋₄ alkyl)(C₁₋₄ alkyl)(C₁₋₄ alkyl)]⁺, such as

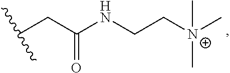 [SMILES: *CC(=O)NCC[N+](C)(C)C]

and any remaining instance(s) of G² is C₁₋₄ alkyl. In some embodiments, T^{A1} can be

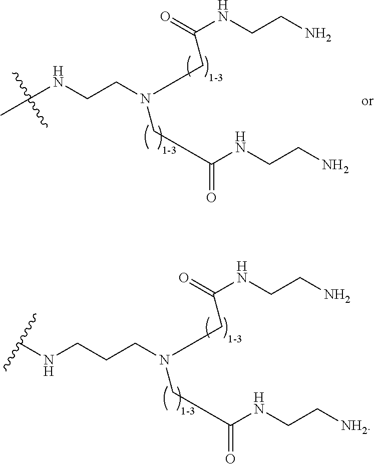

In some embodiments, T^{A1} can be

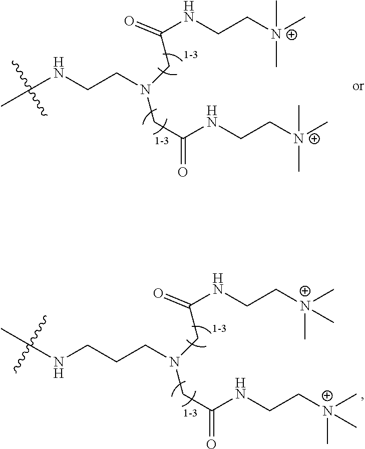

charge balanced with a counterion (e.g., described herein) as necessary. In some embodiments, T^{A1} can be

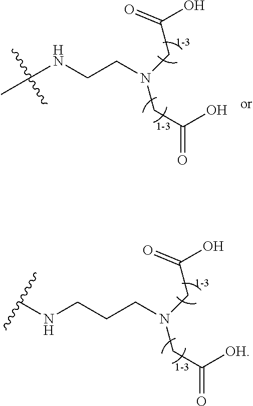

In some embodiments, T^{A1} can be

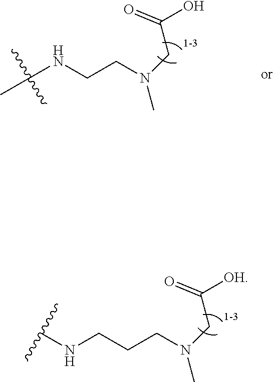

In some embodiments, T^{A1} can be

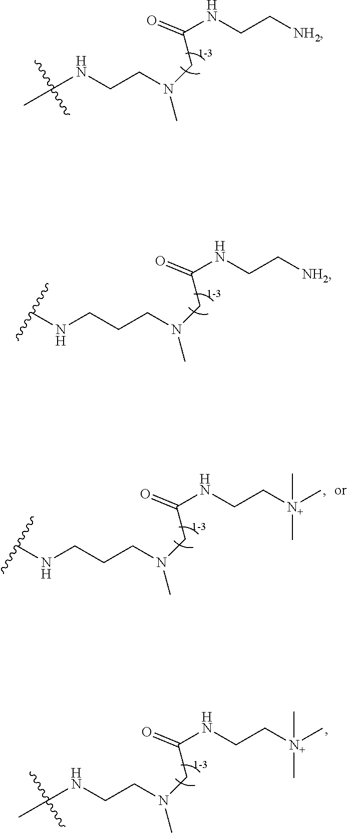

charge balanced with a counterion (e.g., described herein) as necessary. In some embodiments, T^{A1} can be

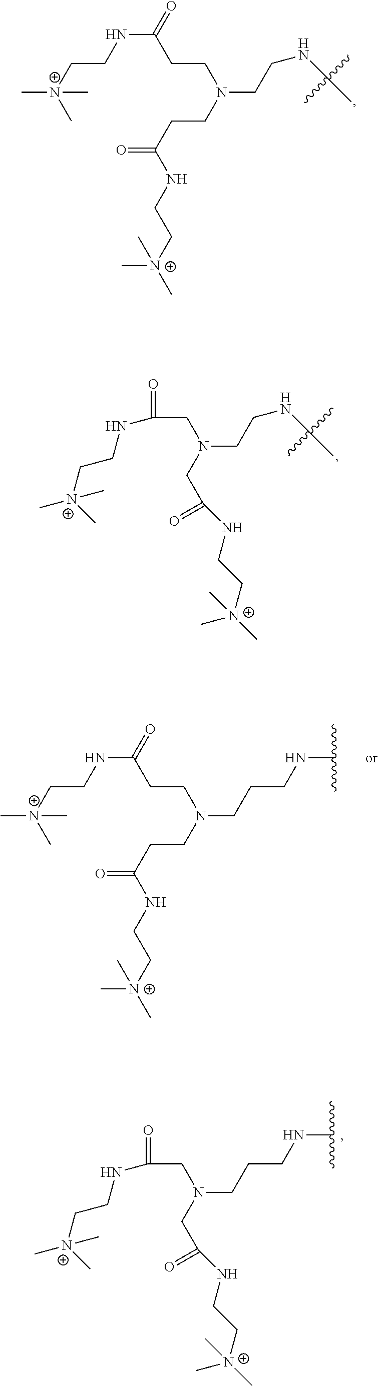 [SMILES: *NCCN(CCC(=O)NCC[N+](C)(C)C)CCC(=O)NCC[N+](C)(C)C ; *NCCN(CC(=O)NCC[N+](C)(C)C)CC(=O)NCC[N+](C)(C)C ; *NCCCN(CCC(=O)NCC[N+](C)(C)C)CCC(=O)NCC[N+](C)(C)C ; *NCCCN(CC(=O)NCC[N+](C)(C)C)CC(=O)NCC[N+](C)(C)C]

charge balanced with a counterion (e.g., described herein) as necessary. For example, in some embodiments, T^{A1} can be

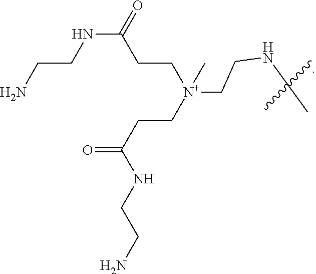 [SMILES: *NCC[N+](C)(CCC(=O)NCCN)CCC(=O)NCCN]

In some embodiments, T^{A1} can be

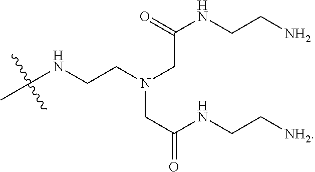 [SMILES: *NCCN(CC(=O)NCCN)CC(=O)NCCN]

In some embodiments, T^{A1} can be

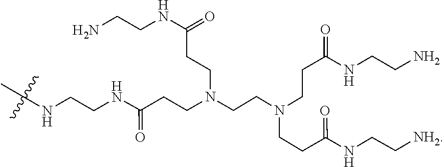 [SMILES: *NCCNC(=O)CCN(CCC(=O)NCCN)CCN(CCC(=O)NCCN)CCC(=O)NCCN]

In some embodiments, T^{A1} can be

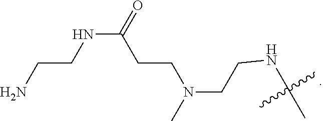 [SMILES: *NCCN(C)CCC(=O)NCCN]

In some embodiments, T^{A1} can be

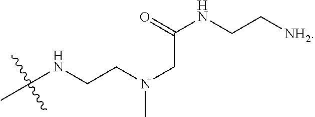 [SMILES: *NCCN(C)CC(=O)NCCN]

In some embodiments, T^{A1} can be

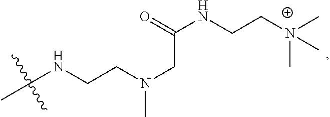 [SMILES: *NCCN(C)CC(=O)NCC[N+](C)(C)C]

charge balanced with a counterion (e.g., described herein) as necessary. In some embodiments, T^{A1} can be

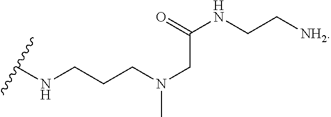 [SMILES: *NCCCN(C)CC(=O)NCCN]

In some embodiments, T^{A1} can be

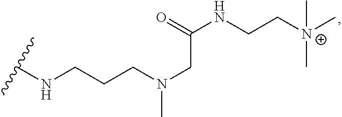 [SMILES: *NCCCN(C)CC(=O)NCC[N+](C)(C)C]

charge balanced with a counterion (e.g., described herein) as necessary. In some embodiments, T^{A1} can be

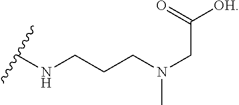 [SMILES: *NCCCN(C)CC(=O)O]

In some embodiments, T^{A} is T^{A1}, T^{A1}-L^{B}-, T^{A1}-L^{B}-(heteroalkylene), or T^{A1}-L^{B}-(heteroalkylene)-L^{B}-, wherein L^{B} at each occurrence is independently —SO₂—, —C(═O)—, or a moiety selected from:

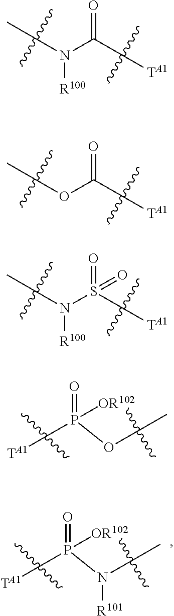

wherein R¹⁰⁰, R¹⁰¹ and R¹⁰² at each occurrence is independently hydrogen, optionally substituted alkyl, or optionally substituted cycloalkyl, wherein T^{A1} is characterized as having a structure of:

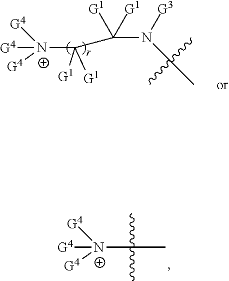

charge balanced with a counterion (e.g., described herein) as necessary, i.e., as needed to maintain overall electronic neutrality of the compound,
wherein G¹ at each occurrence is independently hydrogen, halogen, optionally substituted C₁₋₄ alkyl, or optionally substituted C₁₋₄ heteroalkyl (such as optionally substituted C₁₋₄ alkoxy), or two gem G¹ group can join to form an —O, ═NH, or ═N(C₁₋₄ alkyl), or two or more G¹ groups can join to form a ring structure, the integer r is 1-10, such as 1 or 2,
G³ is hydrogen or an optionally substituted alkyl (e.g., C₁₋₄ alkyl);
G⁴ at each occurrence is independently hydrogen or G¹⁰, wherein G¹⁰ at each occurrence is independently an optionally substituted alkyl, an optionally substituted heteroalkyl, or an optionally substituted 3-10 membered ring structure, which can be carbocyclic, heterocyclic, aromatic, heteroaromatic, or a combination thereof, or two G⁴ or one G⁴ and one G¹ or one G¹ and G³ can be joined to form an optionally substituted 3-10 membered ring structure, wherein the fragment

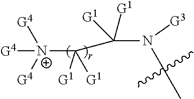

is characterized in that (i) when the CG¹G¹ group next to the NH is not C(═O), the corresponding compound

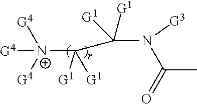

has a cLogP less than 1, preferably, less than 0 (e.g., less than −1, less than −2, less than −3, less than −3.5, less than −4, or even lower), or (ii) when the CG¹G¹ group next to the NH is C(═O), the corresponding compound

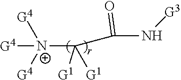

has a cLogP less than 1, preferably, less than 0 (e.g., less than −1, less than −2, less than −3, less than −3.5, less than −4, or even lower). In preferred embodiments, G³ is hydrogen. In some preferred embodiments, T^{A} is T^{A1} or T^{A1}-L^{B}-. In some embodiments, T^{A} is T^{A1}-L^{B}-(heteroalkylene) or T^{A1}-L^{B}-(heteroalkylene)-L^{B}-, wherein the heteroalkylene can be a glycol chain, such as a polyethylene glycol chain.

In some embodiments, T^{A} is T^{A1}. In some embodiments, T^{A} is T^{A1}—C(═O)—. In some embodiments, T^{A1} is characterized as having a structure of:

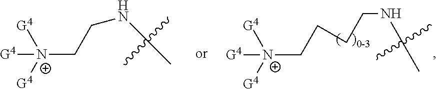

charge balanced with a counterion (e.g., described herein) as necessary. In some embodiments, T^{A1} is characterized as having a structure of:

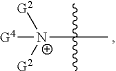

charge balanced with a counterion (e.g., described herein) as necessary. In some embodiments, G⁴ at each occurrence independently can have a structure of

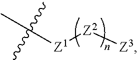

wherein n is an integer of 0-100, preferably, 0-10 (e.g., 0-4, 1-5 or 2-6 etc.), Z¹ is CH₂, C(O), SO₂, CH(C₁₋₄ alkyl), or C(C₁₋₄ alkyl)(C₁₋₄ alkyl), Z² at each occurrence is independently CH₂, O, S, SO, SO₂, C(O), CH(C₁₋₄ alkyl), C(C₁₋₄ alkyl)(C₁₋₄ alkyl), NH, N(C₁₋₄ alkyl), [N(C₁₋₄ alkyl)(C₁₋₄ alkyl)]⁺ or 3-10 membered ring structure, and Z³ is H, C₁₋₄ alkyl, OH, SO₃H, COOH, NH₂, NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, SO₂NH₂, CONH(C₁₋₄ alkyl), CON(C₁₋₄ alkyl)(C₁₋₄ alkyl), SO₂NH(C₁₋₄ alkyl), or SO₂N(C₁₋₄ alkyl)(C₁₋₄ alkyl), wherein each of the C₁₋₄ alkyl is independently selected and optionally substituted, for example, with 1-3 G²⁰ groups defined herein. Preferably, at most 2 instances of Z² is a 3-10 membered ring structure. In some embodiments, none of the Z² is a 3-10 membered ring structure. In some embodiments, one instance of Z² is a 3-10 membered ring structure, such as a nitrogen containing ring, for example,

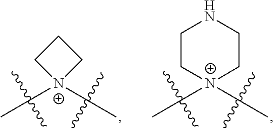 [SMILES: *[N+]1(*)CCC1 ; *[N+]1(*)CCNCC1]

etc. In some embodiments, G⁴ at each occurrence is independently hydrogen, an optionally substituted C₁₋₆ alkyl or an optionally substituted C₁₋₆ heteroalkyl, for example, when substituted, the C₁₋₆ alkyl can be substituted with 1-3 substituents independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), and SO₂N(C₁₋₄ alkyl)(C1-4 alkyl), wherein each of the C₁₋₄ alkyl is independently selected and optionally substituted, for example, with 1-3 G²⁰ groups defined herein. The G²⁰ group herein at each occurrence can be independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), SO₂N(C₁₋₄ alkyl)(C₁₋₄ alkyl), or 3-10 membered ring structure, wherein each of the C₁₋₄ alkyl is independently selected and optionally substituted, for example, with 1-3 G²¹ groups defined herein. The G²¹ group herein at each occurrence can be independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), SO₂N(C₁₋₄ alkyl)(C₁₋₄ alkyl) or 3-10 membered ring structure. In some embodiments, one or more instances of G⁴ is C₁₋₄ alkyl, such as methyl. In some embodiments, one or more instances of G⁴ is C₁₋₄ alkyl, optionally substituted with COOH or an amide derivative, such as CONH—(C₁₋₄ alkylene)-NH₂ or CONH—(C₁₋₄ alkylene)-[N(C₁₋₄ alkyl)(C₁₋₄ alkyl)(C₁₋₄ alkyl)]⁺. For example, in some embodiments, one or two instances of G⁴ is —(C₁₋₄ alkylene)-COOH, such as —CH₂COOH, and any remaining instance(s) of G⁴ is C₁₋₄ alkyl. In some embodiments, one or two instances of G⁴ is —(C₁₋₄ alkylene)-CONH—(C₁₋₄ alkylene)-NH₂, such as

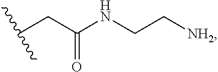 [SMILES: *CC(=O)NCCN]

and any remaining instance(s) of G⁴ is C₁₋₄ alkyl. In some embodiments, one or two instances of G⁴ is —(C₁₋₄ alkylene)-CONH—(C₁₋₄ alkylene)-[N(C₁₋₄ alkyl)(C₁₋₄ alkyl)(C₁₋₄ alkyl)]⁺, such as

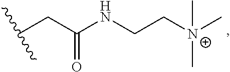 [SMILES: *CC(=O)NCC[N+](C)(C)C]

and any remaining instance(s) of G⁴ is C₁₋₄ alkyl. In some embodiments, T^{A1} can be

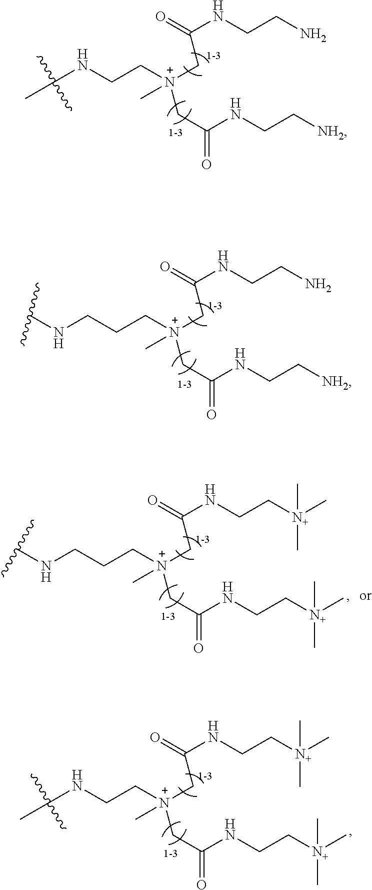

charge balanced with a counterion (e.g., described herein) as necessary. In some embodiments, T^{A1} can be

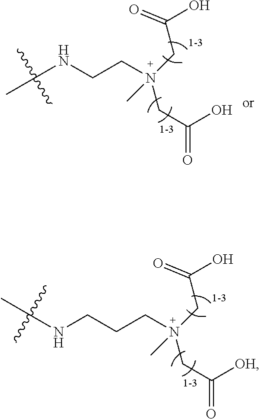

charge balanced with a counterion (e.g., described herein) as necessary. In some embodiments, T^{A1} can be

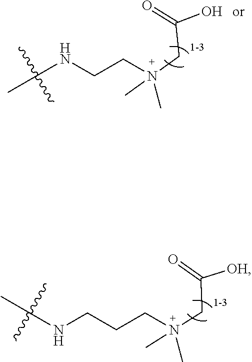

charge balanced with a counterion (e.g., described herein) as necessary. In some embodiments, T^{A1} can be

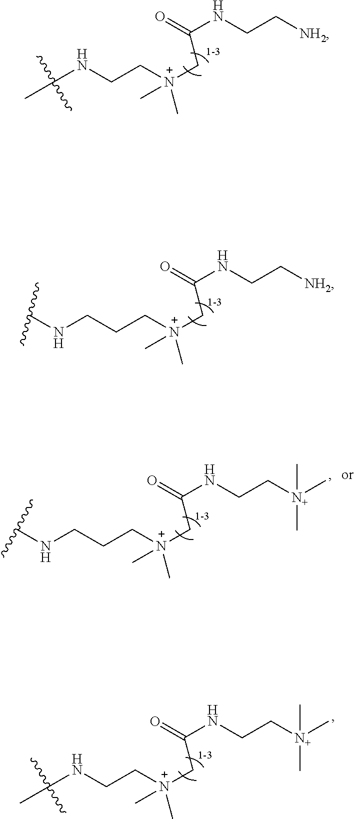

charge balanced with a counterion (e.g., described herein) as necessary. For example, in some embodiments, T^{A1} can be

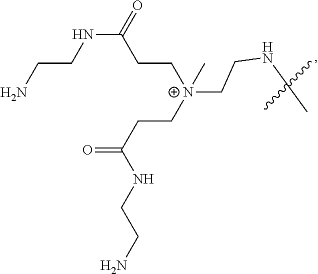 [SMILES: *NCC[N+](C)(CCC(=O)NCCN)CCC(=O)NCCN]

charge balanced with a counterion (e.g., described herein) as necessary. In some embodiments, T^{A1} can be

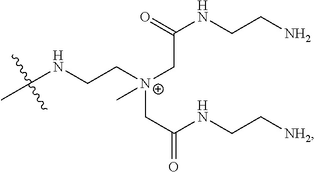 [SMILES: *NCC[N+](C)(CC(=O)NCCN)CC(=O)NCCN]

charge balanced with a counterion (e.g., described herein) as necessary. In some embodiments, T^{A1} can be

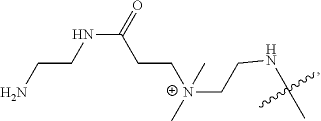 [SMILES: *NCC[N+](C)(C)CCC(=O)NCCN]

charge balanced with a counterion (e.g., described herein) as necessary. In some embodiments, T^{A1} can be

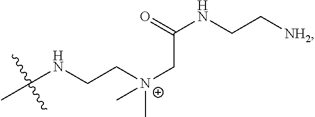 [SMILES: *NCC[N+](C)(C)CC(=O)NCCN]

charge balanced with a counterion (e.g., described herein) as necessary. In some embodiments, T^{A1} can be

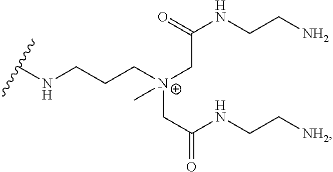 [SMILES: *NCCC[N+](C)(CC(=O)NCCN)CC(=O)NCCN]

charge balanced with a counterion (e.g., described herein) as necessary. In some embodiments, T^{A1} can be

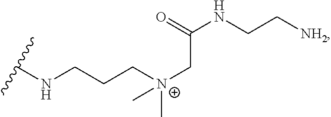 [SMILES: *NCCC[N+](C)(C)CC(=O)NCCN]

charge balanced with a counterion (e.g., described herein) as necessary. In some embodiments, T^{A1} can be

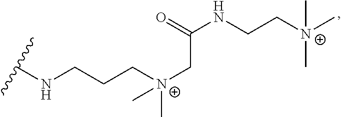 [SMILES: *NCCC[N+](C)(C)CC(=O)NCC[N+](C)(C)C]

charge balanced with a counterion (e.g., described herein) as necessary. In some embodiments, T^{A1} can be

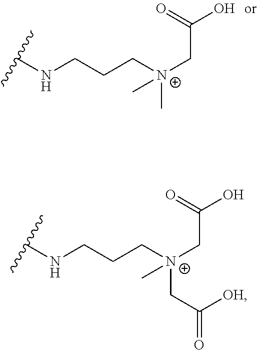 [SMILES: *NCCC[N+](C)(C)CC(=O)O ; *NCCC[N+](C)(CC(=O)O)CC(=O)O]

charge balanced with a counterion (e.g., described herein) as necessary. In some embodiments, T^{A1} can be

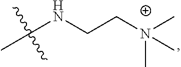

charge balanced with a counterion (e.g., described herein) as necessary. In some embodiments, T^{A1} can be

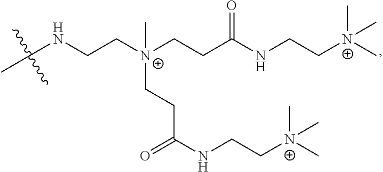 [SMILES: *NCC[N+](C)(CCC(=O)NCC[N+](C)(C)C)CCC(=O)NCC[N+](C)(C)C]

charge balanced with a counterion (e.g., described herein) as necessary. In some embodiments, T^{A1} can be

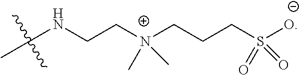

In some preferred embodiments, T^{A} is T^{A1} or T^{A1}-L^{B}-, wherein T^{A1} is

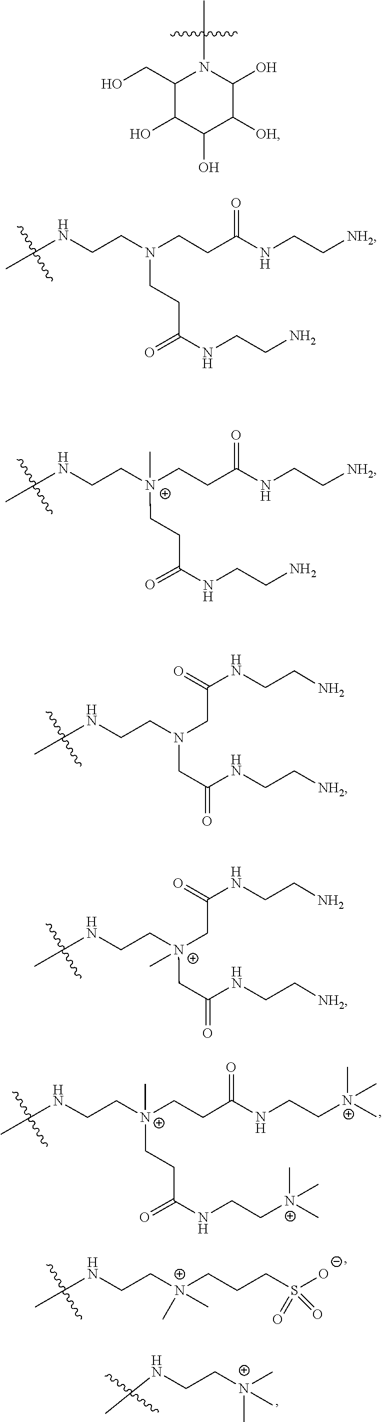

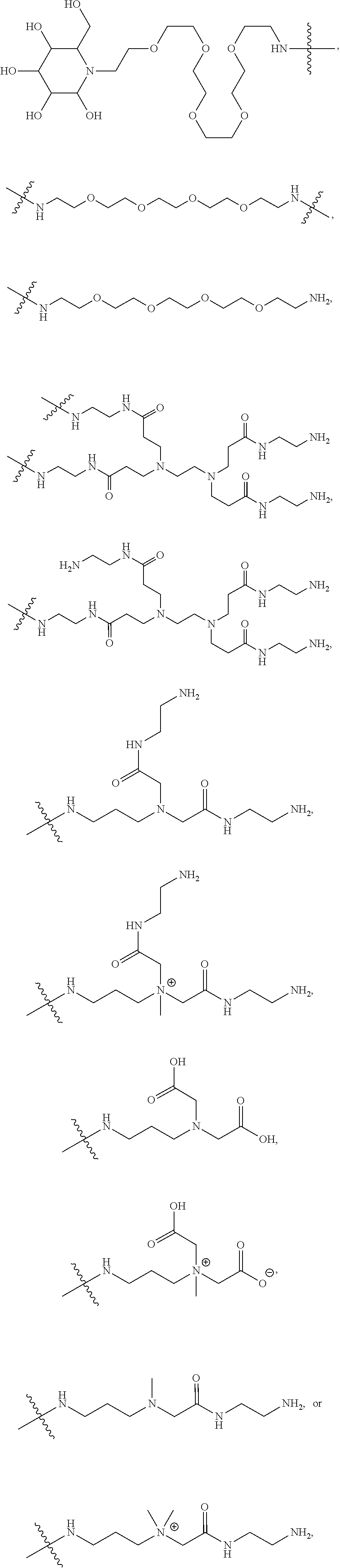

and
wherein L^{B} is —SO₂—, —C(═O)—, or a moiety selected from:

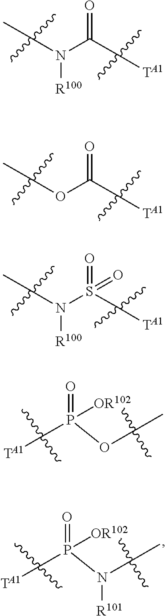

wherein R¹⁰⁰, R¹⁰¹ and R¹⁰² at each occurrence is independently hydrogen, optionally substituted alkyl, or optionally substituted cycloalkyl,
wherein T^{A} is charge balanced with a counterion (e.g., described herein) as necessary.

### Examples of L^{A}-T^{A}

The covalent bond formed between L^{A} and T^{A} is not particularly limited. For example, in some embodiments, the covalent bond(s) between the terminal atom(s) of T^{A} and the end carbon or nitrogen atom(s) of L^{A} is an amide bond. In some embodiments, the covalent bond(s) between the terminal atom(s) of T^{A} and the end atom(s) of L^{A} is a non-amide carbon-nitrogen bond, an ester bond, a non-ester carbon-oxygen bond, a carbon-carbon bond, or a carbon-sulfur bond.

In some preferred embodiments, the compound of Formula I can be characterized as having a Formula I-2, I-3, I-4, I-5, I-6, I-7, I-8, or I-9:

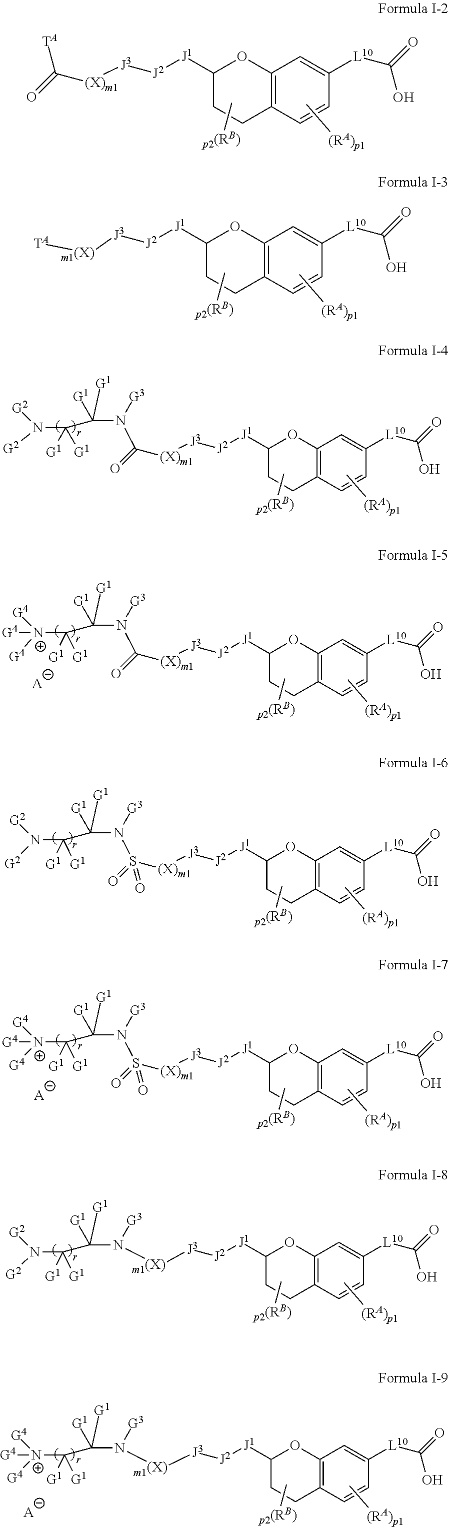

wherein:
X at each occurrence is independently CR₂, C(═O), —C(R)—C(R)—,

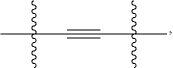 [SMILES: *C#C*]

SiR₂, O, S, SO₂, NR, [NR₂]⁺, or a ring structure, preferably 3-10 membered ring structure, wherein R at each occurrence is independently hydrogen, halogen, optionally substituted C₁₋₄ alkyl, or optionally substituted C₁₋₄ alkoxy,
the integer m1 is at least 12, such as 12-50 (e.g., 12, 14, 16, 18, 20, 22, 24, 30, 40, 50, or any range or value between the recited values),
wherein the hydrophobicity of (X)ₘ₁ is characterized in that (i) when neither of the two end X groups are C(O) or SO₂, then H—(X)ₘ₁—H has a cLogP of at least 3, such as between 3-15, preferably at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc.; (ii) when one end X group is C(O) or SO₂, and the other is not C(O) or SO₂, then the corresponding compound H—(X)ₘ₁—OH has a cLogP of at least 3, such as between 3-15, preferably at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc., the OH being bonded with C(O) or SO₂; or (iii) when both end X groups are C(O) or SO₂, then the corresponding compound HO—(X)ₘ₁—OH has a cLogP of at least 3, such as between 3-15, preferably at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc., the OH being bonded with C(O) or SO₂;
G¹ at each occurrence is independently hydrogen, halogen, optionally substituted C₁₋₄ alkyl, or optionally substituted C₁₋₄ heteroalkyl (such as optionally substituted C₁₋₄ alkoxy), or two gem G¹ group can join to form an ═O, ═NH, or ═N(C₁₋₄ alkyl), or two or more G¹ groups can join to form a ring structure,
the integer r is 1-10, such as 1 or 2,
G² at each occurrence is independently hydrogen, G¹⁰, C(O)-G¹⁰, SO₂G¹⁰, C(O)—NH-G¹⁰, or SO₂NHG¹⁰, C(O)—O-G¹⁰, or SO₂OG¹⁰, wherein G¹⁰ at each occurrence is independently an optionally substituted alkyl, an optionally substituted heteroalkyl, or an optionally substituted 3-10 membered ring structure, which can be carbocyclic, heterocyclic, aromatic, heteroaromatic, or a combination thereof,
G³ is hydrogen or an optionally substituted alkyl (e.g., C₁₋₄ alkyl);
G⁴ at each occurrence is independently hydrogen or G¹⁰, wherein G¹⁰ at each occurrence is independently an optionally substituted alkyl, an optionally substituted heteroalkyl, or an optionally substituted 3-10 membered ring structure, which can be carbocyclic, heterocyclic, aromatic, heteroaromatic, or a combination thereof,
or two G² or one G² and one G¹ or one G¹ and G³ can be joined to form a ring structure such as a 3-10 membered ring structure,
or two G⁴ or one G⁴ and one G¹ or one G¹ and G³ can be joined to form an optionally substituted 3-10 membered ring structure,
A⁻ is a counterion, preferably, a pharmaceutically acceptable anion, as necessary to balance the overall charge of the compound,
wherein the fragment

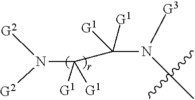

is further characterized in that (i) when the CG¹G¹ group next to the NG³ is not C(═O), the corresponding compound

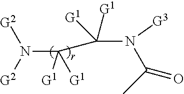

has a cLogP less than 1, preferably, less than 0 (e.g., less than −1, less than −2, less than −3, less than −3.5, less than −4, or even lower), or (ii) when the CG¹G¹ group next to the NG³ is C(═O), the corresponding compound

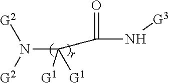

has a cLogP less than 1, preferably, less than 0 (e.g., less than −1, less than −2, less than −3, less than −3.5, less than −4, or even lower),
wherein the fragment

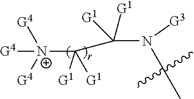

is characterized in that (i) when the CG¹G¹ group next to the NH is not C(═O), the corresponding compound

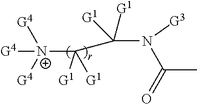

has a cLogP less than 1, preferably, less than 0 (e.g., less than −1, less than −2, less than −3, less than −3.5, less than −4, or even lower), or (ii) when the CG G¹ group next to the NH is C(═O), the corresponding compound has

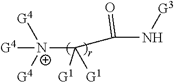

a cLogP less than 1, preferably, less than 0 (e.g., less than −1, less than −2, less than −3, less than −3.5, less than −4, or even lower), and
wherein the variables T^{A}, L¹⁰, J¹, J², J³, R^{A}, R^{B}, p1, and p2 include any of those described and preferred herein in any combinations. In some embodiments, G¹ at each occurrence is hydrogen. In some embodiments, one or more instances of G¹ is a C₁₋₄ alkyl such as methyl. In some embodiments, the integer r is 1, 2, 3, 4, or 5. In some embodiments, one or more instances of G² can have a structure of

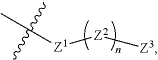

wherein n is an integer of 0-100, preferably, 0-10 (e.g., 0-4, 1-5 or 2-6 etc.), Z¹ is CH₂, C(O), SO, SO₂, CH(C₁₋₄ alkyl), C(C₁₋₄ alkyl)(C₁₋₄ alkyl) or 3-10 membered ring structure, Z² at each occurrence is independently CH₂, O, S, SO, SO₂, C(O), CH(C₁₋₄ alkyl), C(C₁₋₄ alkyl)(C₁₋₄ alkyl), NH, N(C₁₋₄ alkyl), [N(C₁₋₄ alkyl)(C₁₋₄ alkyl)]⁺ or 3-10 membered ring structure, and Z³ is H, C₁₋₄ alkyl, OH, SO₃H, COOH, NH₂, NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, SO₂NH₂, CONH(C₁₋₄ alkyl), CON(C₁₋₄ alkyl)(C₁₋₄ alkyl), SO₂NH(C₁₋₄ alkyl), or SO₂N(C₁₋₄ alkyl)(C₁₋₄ alkyl), wherein each of the C₁₋₄ alkyl is independently selected and optionally substituted, for example, with 1-3 G²⁰ groups defined herein. In some embodiments, none of the Z² is a 3-10 membered ring structure. In some embodiments, one or more instances of Z² is a 3-10 membered ring structure, such as a nitrogen containing ring, for example,

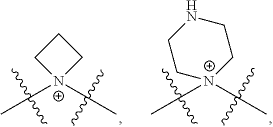 [SMILES: *[N+]1(*)CCC1 ; *[N+]1(*)CCNCC1]

etc. In some embodiments, the Z² directly connects to Z³ is a 3-10 membered ring structure, such as a nitrogen containing ring, for example,

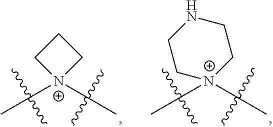 [SMILES: *[N+]1(*)CCC1 ; *[N+]1(*)CCNCC1]

etc. Preferably, at most 2 instances of Z² is a 3-10 membered ring structure. In some embodiments, G² at each occurrence is independently hydrogen, an optionally substituted C₁₋₆ alkyl or an optionally substituted C₁₋₆ heteroalkyl, for example, when substituted, the C₁₋₆ alkyl can be substituted with 1-3 substituents independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), and SO₂N(C₁₋₄ alkyl)(C₁₋₄ alkyl), wherein each of the C₁₋₄ alkyl is independently selected and optionally substituted, for example, with 1-3 G²⁰ groups defined herein. In some embodiments, G⁴ at each occurrence independently can have a structure of

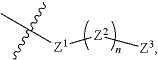

wherein n is an integer of 0-100, preferably, 0-10 (e.g., 0-4, 1-5 or 2-6 etc.), Z¹ is CH₂, C(O), SO₂, CH(C₁₋₄ alkyl), or C(C₁₋₄ alkyl)(C₁₋₄ alkyl), Z² at each occurrence is independently CH₂, O, S, SO, SO₂, C(O), CH(C₁₋₄ alkyl), C(C₁₋₄ alkyl)(C₁₋₄ alkyl), NH, N(C₁₋₄ alkyl), [N(C₁₋₄ alkyl)(C₁₋₄ alkyl)]⁺ or 3-10 membered ring structure, and Z³ is H, C₁₋₄ alkyl, OH, SO₃H, COOH, NH₂, NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, SO₂NH₂, CONH(C₁₋₄ alkyl), CON(C₁₋₄ alkyl)(C₁₋₄ alkyl), SO₂NH(C₁₋₄ alkyl), or SO₂N(C₁₋₄ alkyl)(C₁₋₄ alkyl), wherein each of the C₁₋₄ alkyl is independently selected and optionally substituted, for example, with 1-3 G²⁰ groups defined herein. Preferably, at most 2 instances of Z² is a 3-10 membered ring structure. In some embodiments, none of the Z² is a 3-10 membered ring structure. In some embodiments, one instance of Z² is a 3-10 membered ring structure, such as a nitrogen containing ring, for example,

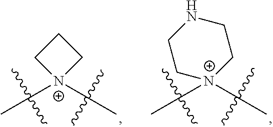 [SMILES: *[N+]1(*)CCC1 ; *[N+]1(*)CCNCC1]

etc. In some embodiments, G⁴ at each occurrence is independently hydrogen, an optionally substituted C₁₋₆ alkyl or an optionally substituted C₁₋₆ heteroalkyl, for example, when substituted, the C₁₋₆ alkyl can be substituted with 1-3 substituents independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), and SO₂N(C₁₋₄ alkyl)(C1-4 alkyl), wherein each of the C₁₋₄ alkyl is independently selected and optionally substituted, for example, with 1-3 G²⁰ groups defined herein. The G²⁰ group herein at each occurrence can be independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), SO₂N(C₁₋₄ alkyl)(C₁₋₄ alkyl), or 3-10 membered ring structure, wherein each of the C₁₋₄ alkyl is independently selected and optionally substituted, for example, with 1-3 G²¹ groups defined herein. The G²¹ group herein at each occurrence can be independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), SO₂N(C₁₋₄ alkyl)(C₁₋₄ alkyl) or 3-10 membered ring structure. In some embodiments, one or more instances of G⁴ is C₁₋₄ alkyl, such as methyl. In some embodiments, one or more instances of G⁴ is C₁₋₄ alkyl, optionally substituted with COOH or an amide derivative, such as CONH—(C₁₋₄ alkylene)-NH₂ or CONH—(C₁₋₄ alkylene)-[N(C₁₋₄ alkyl)(C₁₋₄ alkyl)(C₁₋₄ alkyl)]⁺. For example, in some embodiments, one or two instances of G⁴ is —(C₁₋₄ alkylene)-COOH, such as —CH₂COOH, and any remaining instance(s) of G⁴ is C₁₋₄ alkyl. In some embodiments, one or two instances of G⁴ is —(C₁₋₄ alkylene)-CONH—(C₁₋₄ alkylene)-NH₂, such as

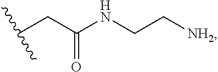 [SMILES: *CC(=O)NCCN]

and any remaining instance(s) of G⁴ is C₁₋₄ alkyl. In some embodiments, one or two instances of G⁴ is —(C₁₋₄ alkylene)-CONH—(C₁₋₄ alkylene)-[N(C₁₋₄ alkyl)(C₁₋₄ alkyl)(C₁₋₄ alkyl)]⁺, such as

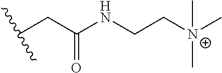 [SMILES: *CC(=O)NCC[N+](C)(C)C]

and any remaining instance(s) of G⁴ is C₁₋₄ alkyl. In preferred embodiments, G³ is hydrogen. In some embodiments, X at each occurrence is independently CH2, CHCH3, or C(CH3)2. In some embodiments, X at each occurrence is CH2. In some embodiments, up to 6 instances (e.g., 1, 2, 3, 4, 5, or 6) of X is O, and the remaining X is CH2, CHCH3, or C(CH3)2. In some embodiments, up to 10 instances of X is —CH═CH—, and the remaining X is CH2, CHCH3, or C(CH3)2. In some embodiments, the moiety of (X)ₘ₁ has one or more structural features selected from the following: (i) two consecutive X together form-C(O)O— or —C(O) NR—(e.g., C(O)NH or C(O)NCH3); (ii) one or more instances of X can have a ring structure selected from cyclopropane, cyclobutane, bicyclobutane [1.1], cyclopentane, cyclohexane or cycloheptane; and (iii) one or more instances of X can have a ring structure selected from phenyl, or 5- or 6-membered heteroaryl, such as triazole. In some embodiments, within the moiety of (X)ₘ₁, there can be 0, 1, or 2 instances of ring structure, preferably 3-10 membered ring structure. In some embodiments, within the moiety of (X)ₘ₁, there is one triazole ring. For example, in some embodiments, the moiety of (X)ₘ₁ can have a structure according to

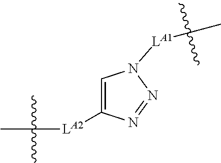

as defined and preferred herein. For example, in some embodiments, LAI and L^{A2} are each independently a bond, —C₁₋₃₀ alkylene-, or —C₁₋₃₀ heteroalkylene-containing 1-6 heteroatoms independently selected from O, N, and S, wherein the sulfur atom(s), if present, is optionally oxidized, wherein the total number of non-hydrogen atoms of (X)ₘ₁ is between 15-50, such as 18, 20, 25, 30, 35, 40, 45, or 50, or any ranges between the recited value. In some embodiments, L^{A1} is a bond. In some embodiments, L^{A1} is a C₁₋₂₀ alkylene, such as a linear alkylene, (CH₂)₁₋₂₀. In some embodiments, L^{A1} is a C₁₋₁₀ heteroalkylene having 1-3 oxygen atoms, such as —O—(C₁₋₁₀ alkylene)-, etc. In some embodiments, L^{A2} is a bond. In some embodiments, L^{A2} is a C₁₋₂₀ alkylene, such as a linear alkylene, (CH₂)₁₋₂₀. In some embodiments, L^{A2} is a C₁₋₁₀ heteroalkylene having 1-3 oxygen atoms, such as —O—(C₁₋₁₀ alkylene)-, etc. Either of L^{A1} and L^{A2} can be attached to J³ in Formula I-2 to I-9 above.

### Residue of GPR40 Agonist

The variables L¹⁰, J¹, J², J³, R^{A}, R^{B}, p1, and p2 in Formula I are not particularly limited. However, in preferred embodiments, the variables in Formula I are such that at least one corresponding compound according to Formula GPR-1,

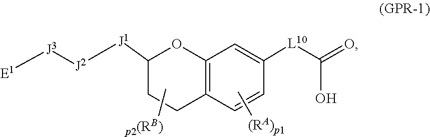

wherein E1 is hydrogen, C₁₋₄ alkyl, N3,

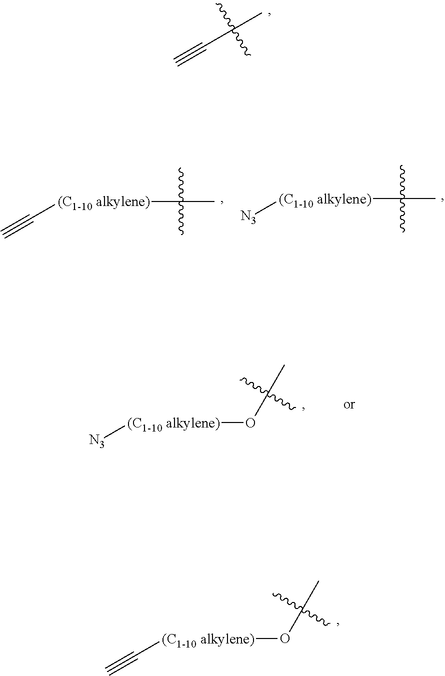

is a GPR40 agonist, preferably, having an EC50 of less than 100 nM as measured according to Biological Example 1 herein.

Typically, p1 in Formula I is 0.

In some embodiments, p1 in Formula I is 1, and R^{A} is F, Cl, CN, C₁₋₄ alkyl optionally substituted with 1-3 fluorine, or C₁₋₄ alkoxy optionally substituted with 1-3 fluorine.

Typically, p2 in Formula I is 0.

In some embodiments, p2 in Formula I is 1 or 2, and R^{B} at each occurrence is independently F, OH, NH₂, NH(C₁₋₄ alkyl), N(C₁₋₄ alkyl)(C₁₋₄ alkyl), C₁₋₄ alkyl optionally substituted with 1-3 fluorine, or C₁₋₄ alkoxy optionally substituted with 1-3 fluorine.

In some preferred embodiments, L¹⁰ is an optionally substituted ethylene. When substituted, the ethylene is typically substituted with one or two substituents, each independently a C₁₋₄ alkyl or a C₃₋₆ cycloalkyl. For example, in some embodiments, L¹⁰ is

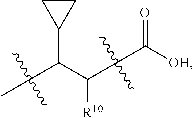

wherein R¹⁰ is hydrogen or C₁₋₄ alkyl. In some preferred embodiments, the compound of Formula I-1 can be characterized as having a formula of

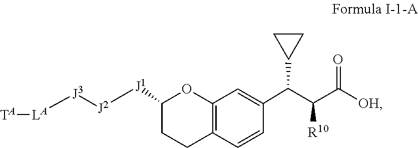

wherein R¹⁰ is hydrogen or C₁₋₄ alkyl (preferably methyl), wherein J¹, J², J³, L^{A}, and T^{A} include any of those described herein in any combinations.

In some embodiments, J¹ in Formula I (e.g., Formula I-1 or I-1-A) is —C₁₋₆alkylene-N(R¹⁰⁰)—, such as —CH₂—N(C₁₋₄ alkyl)-. Typically, in Formula I, J¹ is a 4-12 membered optionally substituted heterocyclic ring having one or two ring nitrogen atoms. In some embodiments, J¹ in Formula I (e.g., Formula I-1 or I-1-A) is a 4-12 membered optionally substituted heterocyclic ring having one or two ring nitrogen atoms. For example, in some embodiments, J¹ is a 4-8 (e.g., 4, 5, 6, or 7) membered monocyclic optionally substituted saturated heterocyclic ring having one or two ring heteroatoms independently selected from S, O, and N, provided at least one of the ring heteroatom is nitrogen. In some embodiments, J¹ is selected from the following (J² is included to show direction of connections):

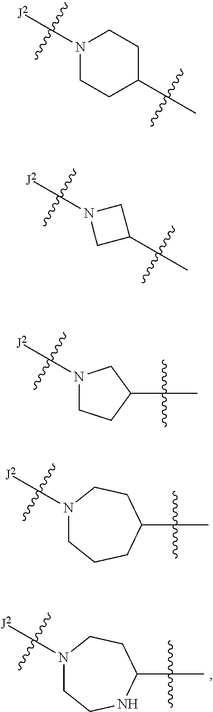

each of which is optionally substituted with 1-2 substituents independently selected from F, OH, NH₂, NH(C₁₋₄ alkyl), N(C₁₋₄ alkyl)(C₁₋₄ alkyl), C₁₋₄ alkyl optionally substituted with 1-3 fluorine, and C₁₋₄ alkoxy optionally substituted with 1-3 fluorine.

In some embodiments, J¹ in Formula I (e.g., Formula I-1 or I-1-A) can also be a bicyclic or polycyclic 6-12 membered optionally substituted saturated heterocyclic ring having one or two ring heteroatoms independently selected from S, O, and N, provided at least one of the ring heteroatom is nitrogen. For example, in some embodiments, J¹ is selected from the following (J² is included to show direction of connections):

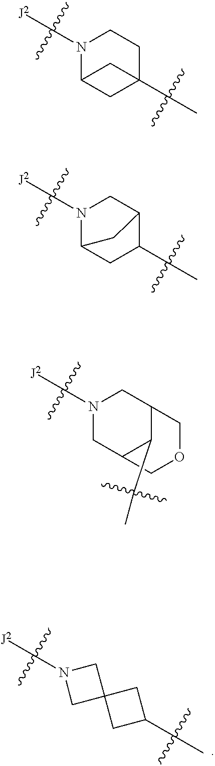

In some embodiments, J² in Formula I (e.g., Formula I-1 or I-1-A) is a straight chain or branched C₁₋₄ alkylene, optionally substituted with 1-3 fluorine. For example, in some embodiments, J² is CH₂ or —CH(CH₃)—.

J³ in Formula I (e.g., Formula I-1 or I-1-A) is typically an aryl (e.g., phenyl) or heteroaryl ring (e.g., pyridyl), each of which is unsubstituted or substituted with one or more (e.g., 1, 2, or 3) substituents independently selected from 1) halogen, CN, —CF₃, OH, amino, substituted amino, ester, amide, carbonate, or carbamate; and 2) C₁₋₆ alkyl, C₂₋₆ alkenyl, C₂₋₆ alkynyl, C₁₋₆ heteroalkyl, C₃₋₆ cycloalkyl, C₁₋₆ alkyl alkoxy, C₃₋₆ cycloalkoxy, aryl, heteroaryl, 3-8 membered heterocycloalkyl having one or two ring heteroatoms independently selected from N, O, and S, wherein each of which is optionally substituted with one or more (e.g., 1, 2, or 3) substituents independently selected from F, —OH, protected hydroxyl, oxo (as applicable), NH₂, protected amino, NH(C₁₋₄ alkyl) or a protected derivative thereof, N(C₁₋₄ alkyl((C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2, or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy.

In some embodiments, J³ in Formula I (e.g., Formula I-1 or I-1-A) is a phenyl ring, which is substituted with 1-3 substituents independently selected from F, Cl, CN, OH, C₁₋₆ alkyl, C₁₋₆ alkyl heteroalkyl, C₃₋₆ cycloalkyl, C₁₋₆ alkyl alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), C₁₋₄ alkyl optionally substituted with F. For example, in some embodiments, the phenyl ring can be substituted with one or two substituents independently selected from C₁₋₄ alkyl optionally substituted with fluorine, e.g., CF₃, and C₁₋₆ alkyl alkoxy optionally substituted with fluorine, such as methoxy, ethoxy, isopropoxy, or O—CF₃.

In some embodiments, J³ in Formula I (e.g., Formula I-1 or I-1-A) is a 5-10 membered monocyclic or bicyclic heteroaryl ring, which is substituted with 1-3 substituents independently selected from F, Cl, CN, OH, C₁₋₆ alkyl, C₁₋₆ alkyl heteroalkyl, C₃₋₆ cycloalkyl, C₁₋₆ alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), C₁₋₄ alkyl optionally substituted with F.

For example, in some embodiments, J³ in Formula I (e.g., Formula I-1 or I-1-A) is selected from:

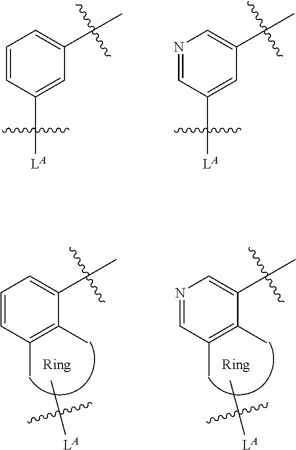

wherein: Ring represents an aromatic or non-aromatic ring structure,
wherein each of the phenyl, pyridyl, or fused ring structure is optionally substituted with 1-3 substituents independently selected from F, Cl, CN, OH, C₁₋₆ alkyl, C₁₋₆ alkyl heteroalkyl, C₃₋₆ cycloalkyl, C₁₋₆ alkyl alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), C₁₋₄ alkyl optionally substituted with F. For example, in some embodiments, the phenyl, pyridyl, or fused ring structure can be substituted with one or two substituents independently selected from C₁₋₄ alkyl optionally substituted with fluorine, e.g., CF₃, and C₁₋₆ alkyl alkoxy optionally substituted with fluorine, such as methoxy, ethoxy, isopropoxy, or O—CF₃.

In some embodiments, J³ in Formula I (e.g., Formula I-1 or I-1-A) is selected from:

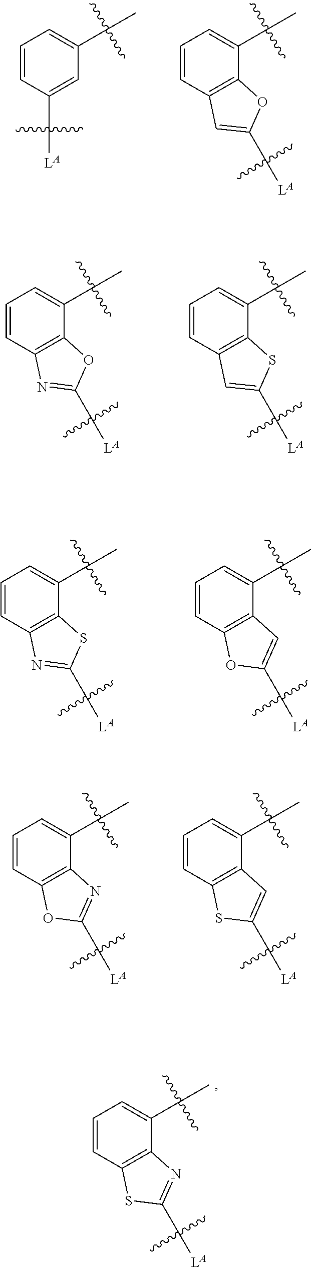

wherein each of which is optionally substituted with 1-3 substituents independently selected from F, Cl, CN, OH, C₁₋₆ alkyl, C₁₋₆ alkyl heteroalkyl, C₃₋₆ cycloalkyl, C₁₋₆ alkyl alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), C₁₋₄ alkyl optionally substituted with F.

In some embodiments, J³ in Formula I (e.g., Formula I-1 or I-1-A) is selected from:

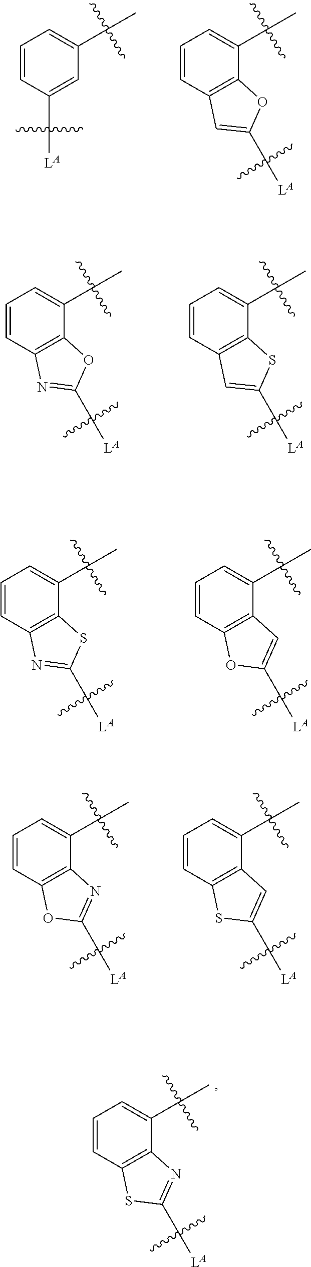

wherein each of which is optionally substituted with 1-3 substituents independently selected from F, Cl, CN, OH, C₁₋₆ alkyl optionally substituted with F (e.g., CF₃), cyclopropyl, cyclobutyl, C₁₋₆ alkyl alkoxy optionally substituted with F (e.g., —O—CF₃), or C₃₋₆ cycloalkoxy. For example, in some embodiments, the phenyl, benzofuran, benzothiophene, benzoxazol, or benzothiazol ring can be substituted with one or two substituents independently selected from C₁₋₄ alkyl optionally substituted with fluorine, e.g., CF₃, C₁₋₆ alkyl alkoxy optionally substituted with fluorine, such as methoxy, ethoxy, isopropoxy, or O—CF₃. Preferably, the one substituent is ortho to J².

In some embodiments, the compound of Formula I is characterized as having a Formula I-1-A-1, I-1-A-2, I-1-A-3, I-1-A-4, or I-1-A-5:

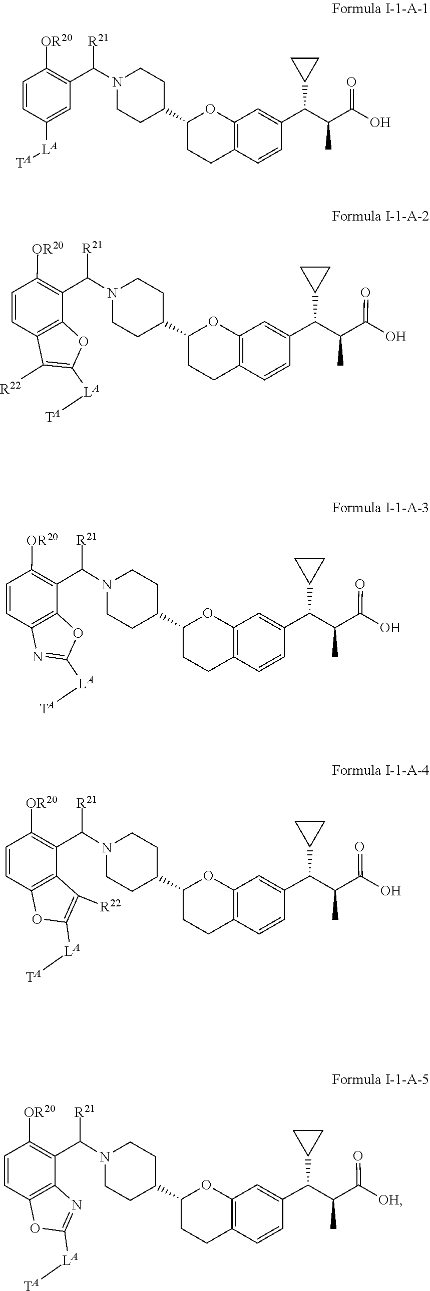

wherein:
R²⁰ is C₁₋₆ alkyl or fluorine substituted C₁₋₆ alkyl, R²¹ is hydrogen or C₁₋₆ alkyl, and R²² is hydrogen, halogen, CN, C₁₋₆ alkyl or fluorine substituted C₁₋₆ alkyl or a C₃₋₆ cycloalkyl, wherein L^{A} and T^{A} include any of those described herein in any combinations. In some embodiments, R²⁰ is methyl, ethyl, n-propyl, isopropyl, or CF₃. In some embodiments, R²¹ is hydrogen, methyl, ethyl, n-propyl, or isopropyl. In some embodiments, R²⁰ is CF₃ and R²¹ is hydrogen or methyl. In some embodiments, R²⁰ is CH₃ and R²¹ is hydrogen or methyl. In some embodiments, R²² is hydrogen. In some embodiments, R²² is methyl. In some embodiments, R²² is cyclopropyl.

In some embodiments, the compound of Formula I is characterized as having a Formula I-1-A-6, I-1-A-7, I-1-A-8, I-1-A-9, or I-1-A-10:

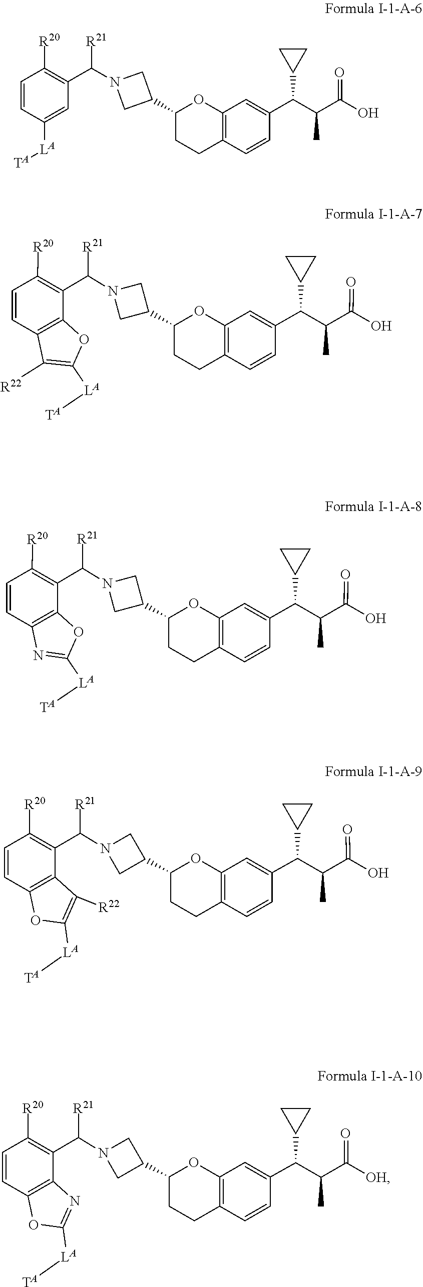

wherein:
R²⁰ is C₁₋₆ alkyl or fluorine substituted C₁₋₆ alkyl, R²¹ is hydrogen or C₁₋₆ alkyl, and R²² is hydrogen, halogen, CN, C₁₋₆ alkyl or fluorine substituted C₁₋₆ alkyl or a C₃₋₆ cycloalkyl, wherein L^{A} and T^{A} include any of those described herein in any combinations. In some embodiments, R²⁰ is methyl, ethyl, n-propyl, isopropyl, or CF₃. In some embodiments, R²¹ is hydrogen, methyl, ethyl, n-propyl, or isopropyl. In some embodiments, R²⁰ is CF₃ and R²¹ is hydrogen or methyl. In some embodiments, R²⁰ is CH₃ and R²¹ is hydrogen or methyl. In some embodiments, R²² is hydrogen. In some embodiments, R²² is methyl. In some embodiments, R²² is cyclopropyl.

In preferred embodiments, L^{A} in Formula I-1-A-1 to I-1-A-10 can be -X12-30-C(O)—, wherein the C(O) is directly bonded with T^{A}, and wherein X at each occurrence is independently CR₂, —C(R)—C(R)—,

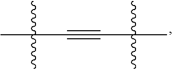 [SMILES: *C#C*]

SiR₂, C(O), O, NR, S, SO₂, or a ring structure, preferably 3-10 membered ring structure, wherein R at each occurrence is independently hydrogen, halogen, optionally substituted C₁₋₄ alkyl, or optionally substituted C₁₋₄ alkoxy, typically hydrogen or C₁₋₄ alkyl, wherein the longest chain length of L^{A} is at least that of —(CH₂)₁₂, such as at least that of —(CH₂)₁₄, at least that of —(CH₂)₁₆, at least that of —(CH₂)₁₈, and wherein the hydrophobicity of L^{A} can be characterized in that the corresponding compound H—X₁₂₋₃₀—C(O)—OH has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc. Preferably, the end X group is not C(═O) or SO₂. Preferably, the total number of non-hydrogen atoms of L^{A} is between 15-50, such as 18, 20, 25, 30, 35, 40, 45, or 50, or any ranges between the recited value. In some preferred embodiments, none of the X groups is a ring structure. In some preferred embodiments, one instance of X is a ring structure, preferably 3-10 membered ring structure. For example, in some embodiments, one instance of X is triazole and the remaining X groups are each independently CR₂, —C(R)—C(R)—,

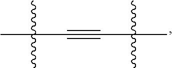 [SMILES: *C#C*]

or O, wherein R at each occurrence is independently hydrogen or C₁₋₄ alkyl. In some embodiments, none of the X groups is a ring structure, and each X is independently CR₂, —C(R)═C(R)—,

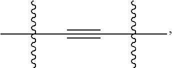 [SMILES: *C#C*]

or O, wherein R at each occurrence is independently hydrogen or C₁₋₄ alkyl (preferably, methyl). In some embodiments, all instances of X is CR₂, wherein R at each occurrence is independently hydrogen or C₁₋₄ alkyl (preferably, methyl). In some embodiments, up to 6 instances (e.g., 1, 2, 3, 4, 5, or 6) of X can be O. In some embodiments, one or two instances of X is O, and all other instances of X is CR₂, wherein R at each occurrence is independently hydrogen or C₁₋₄ alkyl (preferably, methyl). In some embodiments, one or two instances of X is O, one X is triazole, and all other instances of X is CR₂, wherein R at each occurrence is independently hydrogen or C₁₋₄ alkyl (preferably, methyl). In some embodiments, R is hydrogen.

In some preferred embodiments, T^{A} in Formula I-1-A-1 to I-1-A-10 can be

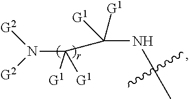

wherein G¹ at each occurrence is independently hydrogen, halogen, optionally substituted C₁₋₄ alkyl, or optionally substituted C₁₋₄ heteroalkyl (such as optionally substituted C₁₋₄ alkoxy), or two gem G¹ group can join to form an ═O, ═NH, or ═N(C₁₋₄ alkyl), or two or more G¹ groups can join to form a ring structure,
the integer r is 1-10, such as 1 or 2,
G² at each occurrence is independently hydrogen, G¹⁰, C(O)-G¹⁰, SO₂G¹⁰, C(O)—NH-G¹⁰, or SO₂NHG¹⁰, C(O)—O-G¹⁰, or SO₂OG¹⁰, wherein G¹⁰ at each occurrence is independently an optionally substituted C₁₋₆ alkyl, an optionally substituted C₁₋₆ heteroalkyl, or an optionally substituted 3-10 membered ring structure, which can be carbocyclic, heterocyclic, aromatic, heteroaromatic, or a combination thereof, wherein the fragment is further characterized in that (i) when the CG¹G¹ group next to the NH is not C(═O), the corresponding compound

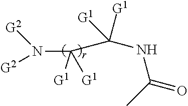

has a cLogP less than 1, preferably, less than 0 (e.g., less than −1, less than −2, less than −3, less than −3.5, less than −4, or even lower), or (ii) when the CG¹G¹ group next to the NH is C(═O), the corresponding compound

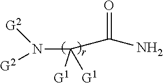

has a cLogP less than 1, preferably, less than 0 (e.g., less than −1, less than −2, less than −3, less than −3.5, less than −4, or even lower).

In some preferred embodiments, T^{A} in Formula I-1-A-1 to I-1-A-10 can be characterized as having a structure of:

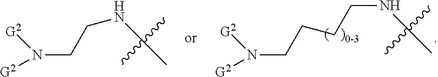

In some embodiments, one or both instances of G² can have a structure of

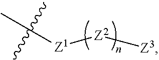

wherein n is an integer of 0-100, preferably, 0-10 (e.g., 0-4, 1-5 or 2-6 etc.), Z¹ is CH₂, C(O), SO, SO₂, CH(C₁₋₄ alkyl), C(C₁₋₄ alkyl)(C₁₋₄ alkyl) or 3-10 membered ring structure, Z² at each occurrence is independently CH₂, O, S, SO, SO₂, C(O), CH(C₁₋₄ alkyl), C(C₁₋₄ alkyl)(C₁₋₄ alkyl), NH, N(C₁₋₄ alkyl), [N(C₁₋₄ alkyl)(C₁₋₄ alkyl)]⁺ or 3-10 membered ring structure, and Z³ is H, C₁₋₄ alkyl, OH, SO₃H, COOH, NH₂, NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, SO₂NH₂, CONH(C₁₋₄ alkyl), CON(C₁₋₄ alkyl)(C₁₋₄ alkyl), SO₂NH(C₁₋₄ alkyl), or SO₂N(C₁₋₄ alkyl)(C₁₋₄ alkyl), wherein each of the C₁₋₄ alkyl is independently selected and optionally substituted, for example, with 1-3 G²⁰ groups defined herein. In some embodiments, none of the Z² is a 3-10 membered ring structure. In some embodiments, one or more instances of Z² is a 3-10 membered ring structure, such as a nitrogen containing ring, for example,

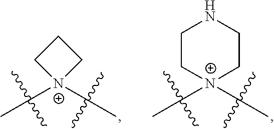 [SMILES: *[N+]1(*)CCC1 ; *[N+]1(*)CCNCC1]

etc. In some embodiments, the Z² directly connects to Z³ is a 3-10 membered ring structure, such as a nitrogen containing ring, for example,

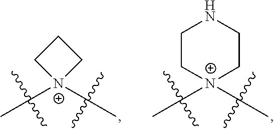 [SMILES: *[N+]1(*)CCC1 ; *[N+]1(*)CCNCC1]

etc. Preferably, at most 2 instances of Z² is a 3-10 membered ring structure. In some embodiments, G² at each occurrence is independently hydrogen, an optionally substituted C₁₋₆ alkyl or an optionally substituted C₁₋₆ heteroalkyl, for example, when substituted, the C₁₋₆ alkyl can be substituted with 1-3 substituents independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl) (C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), and SO₂N(C₁₋₄ alkyl)(C₁₋₄ alkyl), wherein each of the C₁₋₄ alkyl is independently selected and optionally substituted, for example, with 1-3 G²⁰ groups defined herein. The G²⁰ group herein at each occurrence can be independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl) (C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), SO₂N(C₁₋₄ alkyl)(C₁₋₄ alkyl), or 3-10 membered ring structure, wherein each of the C₁₋₄ alkyl is independently selected and optionally substituted, for example, with 1-3 G²¹ groups defined herein. The G²¹ group herein at each occurrence can be independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), SO₂N(C₁₋₄ alkyl)(C₁₋₄ alkyl) or 3-10 membered ring structure. In some embodiments, one or more instances of G² is C₁₋₄ alkyl, optionally substituted with COOH or an amide derivative, such as CONH—(C₁₋₄ alkylene)-NH₂ or CONH—(C₁₋₄ alkylene)-[N(C₁₋₄ alkyl)(C₁₋₄ alkyl)(C₁₋₄ alkyl)]⁺. For example, in some embodiments, one or two instances of G² is —(C₁₋₄ alkylene)-COOH, such as —CH₂COOH, and any remaining instance(s) of G² is C₁₋₄ alkyl. In some embodiments, one or two instances of G² is —(C₁₋₄ alkylene)-CONH—(C₁₋₄ alkylene)-NH₂, such as

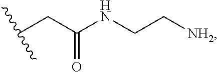 [SMILES: *CC(=O)NCCN]

and any remaining instance(s) of G² is C₁₋₄ alkyl. In some embodiments, one or two instances of G² is —(C₁₋₄ alkylene)-CONH—(C₁₋₄ alkylene)-[N(C₁₋₄ alkyl)(C₁₋₄ alkyl)(C₁₋₄ alkyl)]⁺, such as

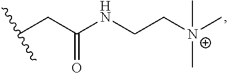 [SMILES: *CC(=O)NCC[N+](C)(C)C]

and any remaining instance(s) of G² is C₁₋₄ alkyl. In some embodiments, T^{A} can be

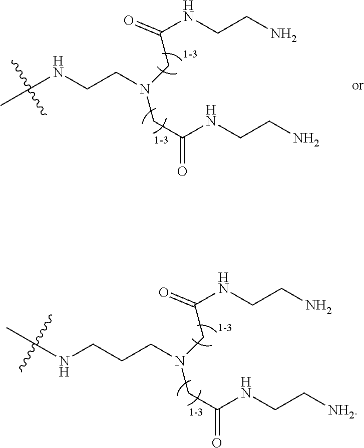

In some embodiments, T^{A} can be

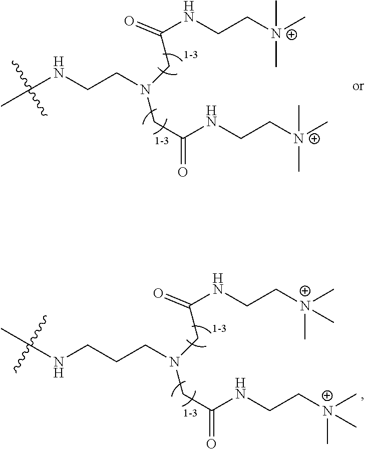

charge balanced with a counterion (e.g., described herein) as necessary. In some embodiments, T^{A} can be

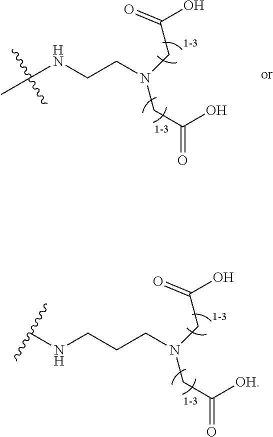

In some embodiments, T^{A} can be

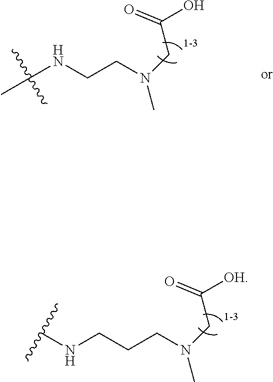

In some embodiments, T^{A} can be

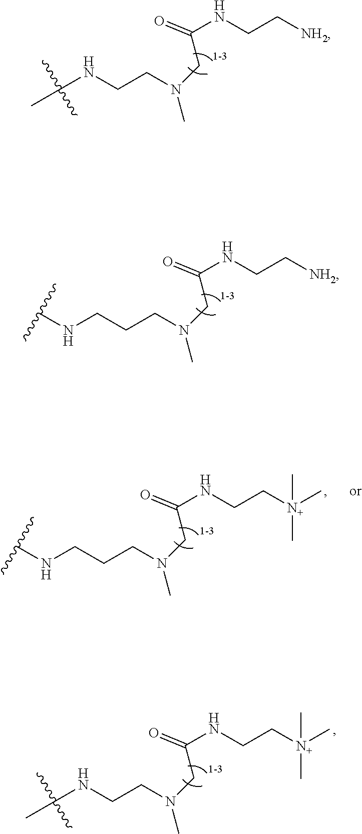

charge balanced with a counterion (e.g., described herein) as necessary. In some embodiments, T^{A} can be

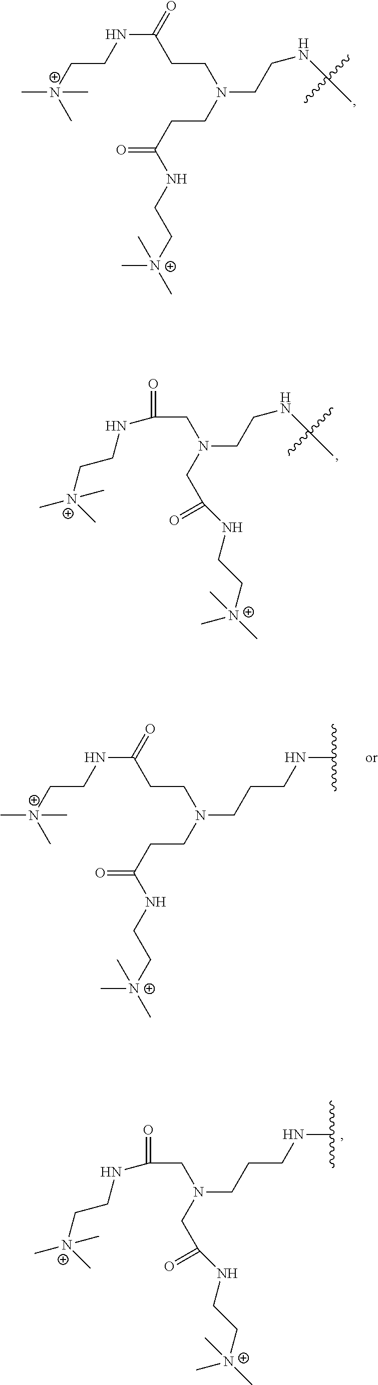 [SMILES: *NCCN(CCC(=O)NCC[N+](C)(C)C)CCC(=O)NCC[N+](C)(C)C ; *NCCN(CC(=O)NCC[N+](C)(C)C)CC(=O)NCC[N+](C)(C)C ; *NCCCN(CCC(=O)NCC[N+](C)(C)C)CCC(=O)NCC[N+](C)(C)C ; *NCCCN(CC(=O)NCC[N+](C)(C)C)CC(=O)NCC[N+](C)(C)C]

charge balanced with a counterion (e.g., described herein) as necessary. In some embodiments, T^{A} can be

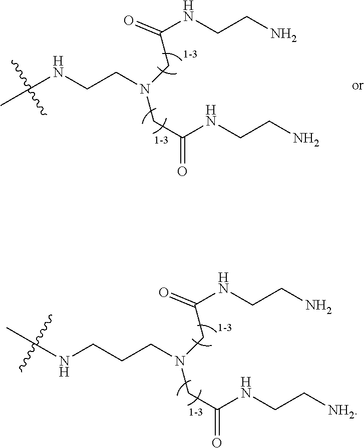

For example, in some embodiments, T^{A} can be

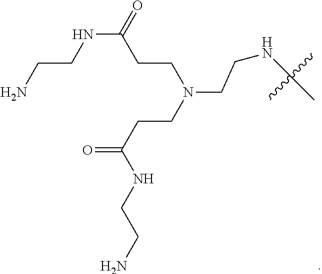 [SMILES: *NCCN(CCC(=O)NCCN)CCC(=O)NCCN]

In some embodiments, T^{A} can be

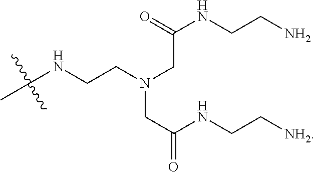 [SMILES: *NCCN(CC(=O)NCCN)CC(=O)NCCN]

In some embodiments, T^{A} can be

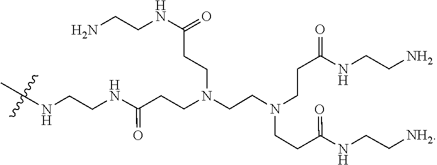 [SMILES: *NCCNC(=O)CCN(CCC(=O)NCCN)CCN(CCC(=O)NCCN)CCC(=O)NCCN]

In some embodiments, T^{A} can be

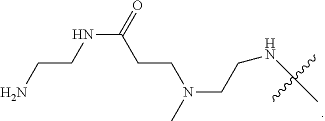 [SMILES: *NCCN(C)CCC(=O)NCCN]

In some embodiments, T^{A} can be

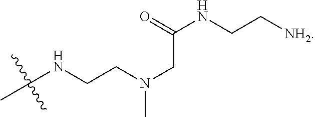 [SMILES: *NCCN(C)CC(=O)NCCN]

In some embodiments, T^{A} can be

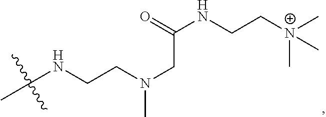 [SMILES: *NCCN(C)CC(=O)NCC[N+](C)(C)C]

charge balanced with a counterion (e.g., described herein) as necessary. In some embodiments, T^{A} can be

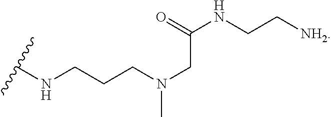 [SMILES: *NCCCN(C)CC(=O)NCCN]

In some embodiments, T^{A} can be

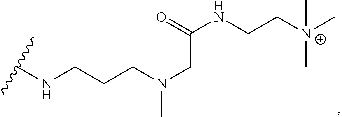 [SMILES: *NCCCN(C)CC(=O)NCC[N+](C)(C)C]

charge balanced with a counterion (e.g., described herein) as necessary. In some embodiments, T^{A} can be

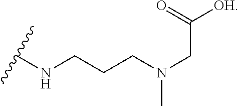 [SMILES: *NCCCN(C)CC(=O)O]

In some preferred embodiments, T^{A} in Formula I-1-A-1 to I-1-A-10 can be characterized as having a structure of:

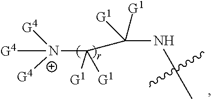

charge balanced with a counterion (e.g., described herein) as necessary, wherein G¹ at each occurrence is independently hydrogen, halogen, optionally substituted C₁₋₄ alkyl, or optionally substituted C₁₋₄ heteroalkyl (such as optionally substituted C₁₋₄ alkoxy), or two gem G¹ group can join to form an —O, ═NH, or ═N(C₁₋₄ alkyl), or two or more G¹ groups can join to form a ring structure,
the integer r is 1-10, such as 1 or 2,
G⁴ at each occurrence is independently hydrogen or G¹⁰, wherein G¹⁰ at each occurrence is independently an optionally substituted C₁₋₆ alkyl, an optionally substituted C₁₋₆ heteroalkyl, or an optionally substituted 3-10 membered ring structure, which can be carbocyclic, heterocyclic, aromatic, heteroaromatic, or a combination thereof, wherein the fragment

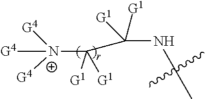

is characterized in that (i) when the CG¹G¹ group next to the NH is not C(═O), the corresponding compound

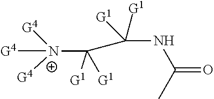

has a cLogP less than 1, preferably, less than 0 (e.g., less than −1, less than −2, less than −3, less than −3.5, less than −4, or even lower), or (ii) when the CG¹G¹ group next to the NH is C(═O), the corresponding compound

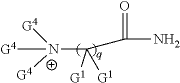

has a cLogP less than 1, preferably, less than 0 (e.g., less than −1, less than −2, less than −3, less than −3.5, less than −4, or even lower).

In some preferred embodiments, T^{A} in Formula I-1-A-1 to I-1-A-10 can be characterized as having a structure of:

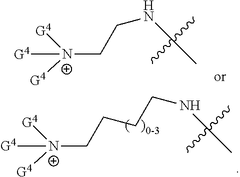

In some embodiments, one or more instances of G⁴ can have a structure of

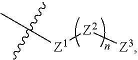

n is an integer of 0-100, preferably, 0-10 (e.g., 0-4, 1-5 or 2-6 etc.), Z¹ is CH₂, C(O), SO₂, CH(C₁₋₄ alkyl), or C(C₁₋₄ alkyl)(C₁₋₄ alkyl), Z² at each occurrence is independently CH₂, O, S, SO, SO₂, C(O), CH(C₁₋₄ alkyl), C(C₁₋₄ alkyl)(C₁₋₄ alkyl), NH, N(C₁₋₄ alkyl), [N(C₁₋₄ alkyl)(C₁₋₄ alkyl)]⁺ or 3-10 membered ring structure, and Z³ is H, C₁₋₄ alkyl, OH, SO₃H, COOH, NH₂, NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, SO₂NH₂, CONH(C₁₋₄ alkyl), CON(C₁₋₄ alkyl)(C₁₋₄ alkyl), SO₂NH(C₁₋₄ alkyl), or SO₂N(C₁₋₄ alkyl)(C₁₋₄ alkyl), wherein each of the C₁₋₄ alkyl is independently selected and optionally substituted, for example, with 1-3 G²⁰ groups defined herein. Preferably, at most 2 instances of Z² is a 3-10 membered ring structure. In some embodiments, none of the Z² is a 3-10 membered ring structure. In some embodiments, one instance of Z² is a 3-10 membered ring structure, such as a nitrogen containing ring, for example,

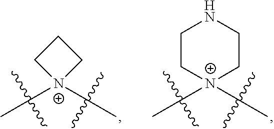 [SMILES: *[N+]1(*)CCC1 ; *[N+]1(*)CCNCC1]

etc. In some embodiments, G⁴ at each occurrence is independently hydrogen, an optionally substituted C₁₋₆ alkyl or an optionally substituted C₁₋₆ heteroalkyl, for example, when substituted, the C₁₋₆ alkyl can be substituted with 1-3 substituents independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), and SO₂N(C₁₋₄ alkyl)(C₁₋₄ alkyl), wherein each of the C₁₋₄ alkyl is independently selected and optionally substituted, for example, with 1-3 G²⁰ groups defined herein. The G²⁰ group herein at each occurrence can be independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), SO₂N(C₁₋₄ alkyl)(C₁₋₄ alkyl), or 3-10 membered ring structure, wherein each of the C₁₋₄ alkyl is independently selected and optionally substituted, for example, with 1-3 G²¹ groups defined herein. The G²¹ group herein at each occurrence can be independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), SO₂N(C₁₋₄ alkyl)(C₁₋₄ alkyl) or 3-10 membered ring structure. In some embodiments, one or more instances of G⁴ is C₁₋₄ alkyl, such as methyl. In some embodiments, one or more instances of G⁴ is C₁₋₄ alkyl, optionally substituted with COOH or an amide derivative, such as CONH—(C₁₋₄ alkylene)-NH₂ or CONH—(C₁₋₄ alkylene)-[N(C₁₋₄ alkyl)(C₁₋₄ alkyl)(C₁₋₄ alkyl)]⁺. For example, in some embodiments, one or two instances of G⁴ is —(C₁₋₄ alkylene)-COOH, such as —CH₂COOH, and any remaining instance(s) of G⁴ is C₁₋₄ alkyl. In some embodiments, one or two instances of G⁴ is —(C₁₋₄ alkylene)-CONH—(C₁₋₄ alkylene)-NH₂, such as

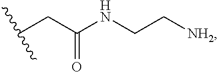 [SMILES: *CC(=O)NCCN]

and any remaining instance(s) of G⁴ is C₁₋₄ alkyl. In some embodiments, one or two instances of G⁴ is (C₁₋₄ alkylene)-CONH—(C₁₋₄ alkylene)-[N(C₁₋₄ alkyl)(C₁₋₄ alkyl)(C₁₋₄ alkyl)]⁺, such as

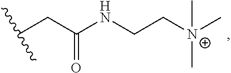 [SMILES: *CC(=O)NCC[N+](C)(C)C]

and any remaining instance(s) of G⁴ is C₁₋₄ alkyl. In some embodiments, T^{A} can be

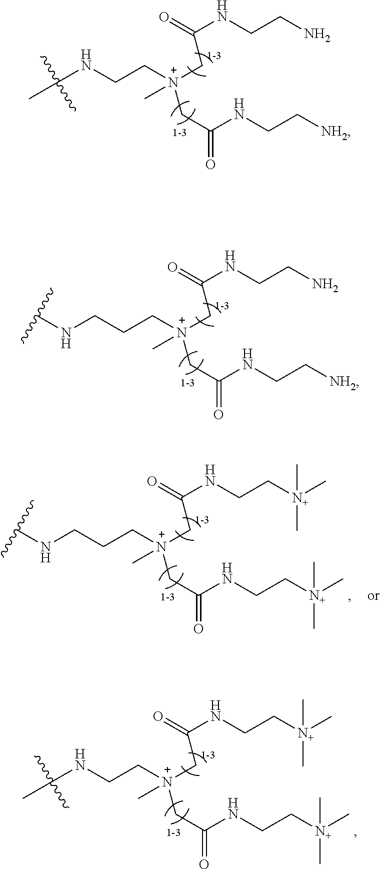

charge balanced with a counterion (e.g., described herein) as necessary. In some embodiments, T^{A} can be

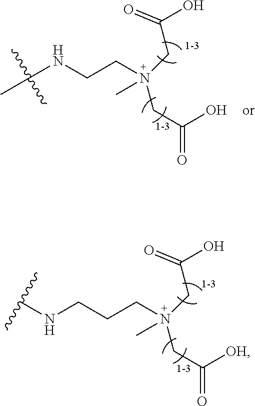

charge balanced with a counterion (e.g., described herein) as necessary. In some embodiments, T^{A} can be

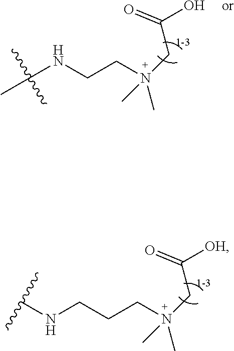

charge balanced with a counterion (e.g., described herein) as necessary. In some embodiments, T^{A} can be

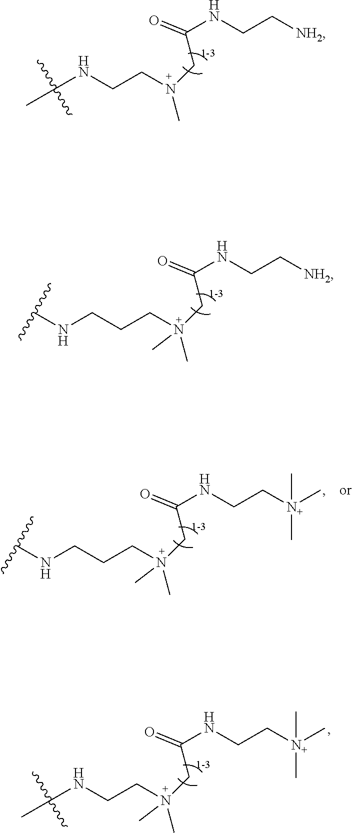

charge balanced with a counterion (e.g., described herein) as necessary. For example, in some embodiments, T^{A} can be

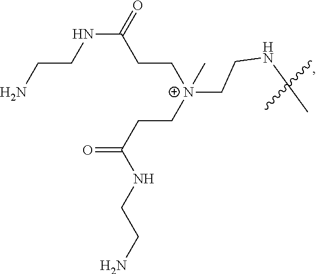 [SMILES: *NCC[N+](C)(CCC(=O)NCCN)CCC(=O)NCCN]

charge balanced with a counterion (e.g., described herein) as necessary. In some embodiments, T^{A} can be

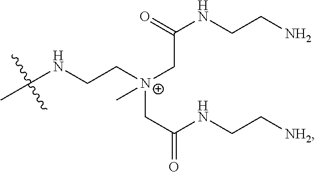 [SMILES: *NCC[N+](C)(CC(=O)NCCN)CC(=O)NCCN]

charge balanced with a counterion (e.g., described herein) as necessary. In some embodiments, T^{A} can be

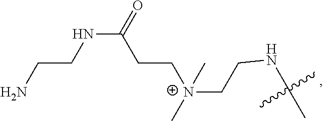 [SMILES: *NCC[N+](C)(C)CCC(=O)NCCN]

charge balanced with a counterion (e.g., described herein) as necessary. In some embodiments, T^{A} can be

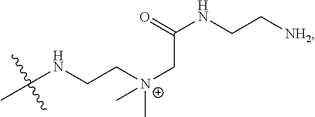 [SMILES: *NCC[N+](C)(C)CC(=O)NCCN]

charge balanced with a counterion (e.g., described herein) as necessary. In some embodiments, T^{A} can be

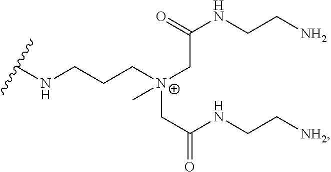 [SMILES: *NCCC[N+](C)(CC(=O)NCCN)CC(=O)NCCN]

charge balanced with a counterion (e.g., described herein) as necessary. In some embodiments, T^{A} can be

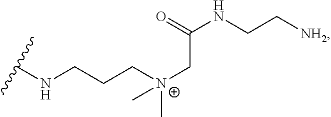 [SMILES: *NCCC[N+](C)(C)CC(=O)NCCN]

charge balanced with a counterion (e.g., described herein) as necessary. In some embodiments, T^{A} can be

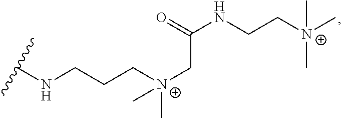 [SMILES: *NCCC[N+](C)(C)CC(=O)NCC[N+](C)(C)C]

charge balanced with a counterion (e.g., described herein) as necessary. In some embodiments, T^{A} can be

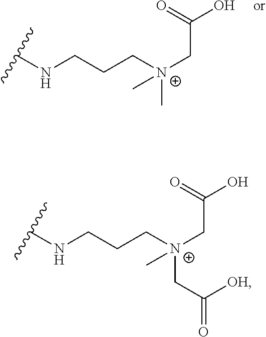 [SMILES: *NCCC[N+](C)(C)CC(=O)O ; *NCCC[N+](C)(CC(=O)O)CC(=O)O]

charge balanced with a counterion (e.g., described herein) as necessary. In some embodiments, T^{A} can be

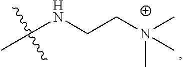 [SMILES: *NCC[N+](C)(C)C]

charge balanced with a counterion (e.g., described herein) as necessary. In some embodiments, T^{A} can be

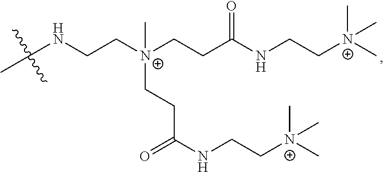 [SMILES: *NCC[N+](C)(CCC(=O)NCC[N+](C)(C)C)CCC(=O)NCC[N+](C)(C)C]

charge balanced with a counterion (e.g., described herein) as necessary. In some embodiments, T^{A} can be

In some preferred embodiments, the present disclosure also provides a compound of Formula I-4-A, I-4-B, I-4-C, I-5-A, I-5-B, or I-5-C, or a pharmaceutically acceptable salt or ester thereof:

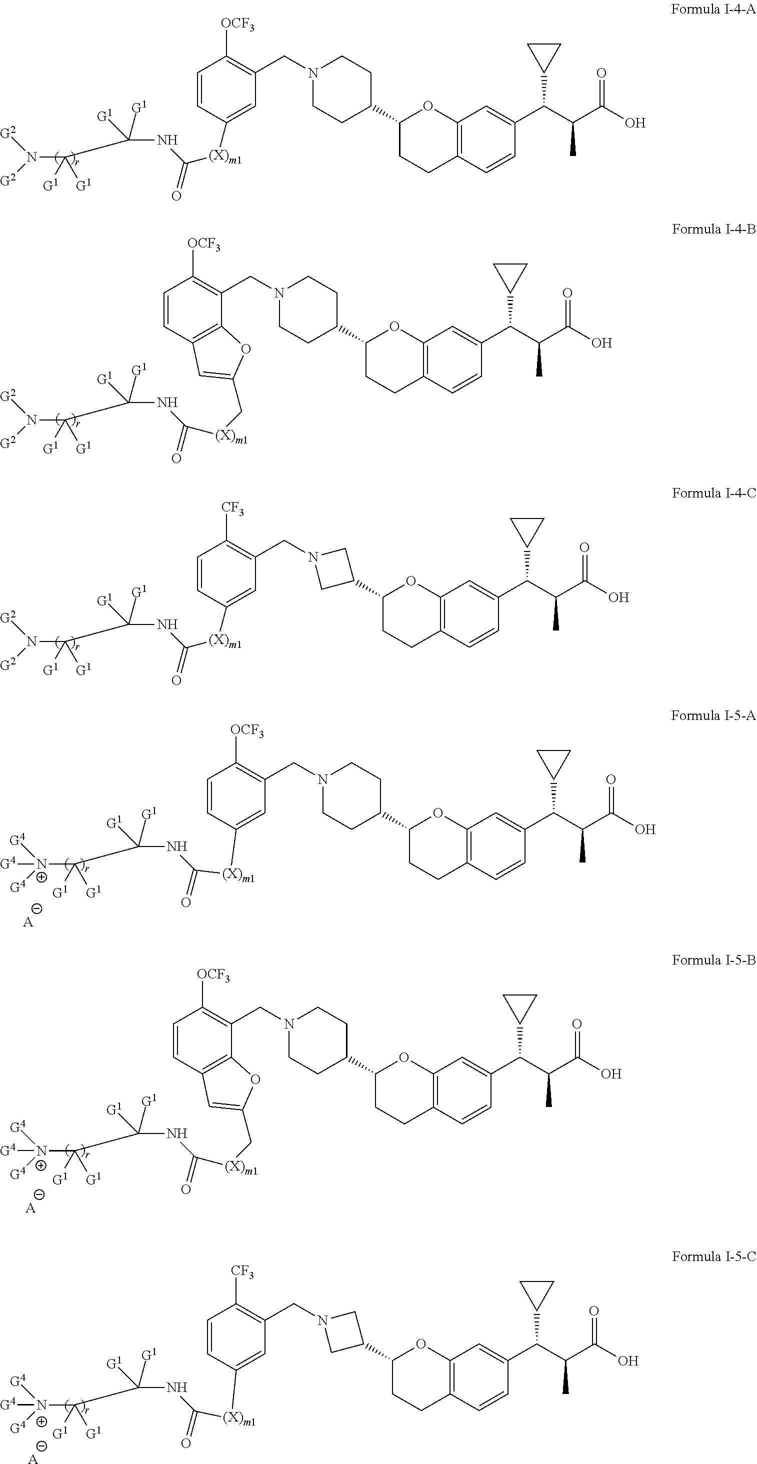

wherein X, m1, G¹, G², G⁴, r, and A⁻ include any of those described and preferred herein, for example, any of those described and preferred in connection with Formula I-4 or I-5, in any combinations. For example, X at each occurrence can be independently CR₂, —C(R)═C(R)—,

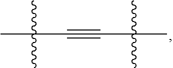 [SMILES: *C#C*]

SiR₂, C(O), O, NR, S, SO₂, or a ring structure, preferably 3-10 membered ring structure, wherein R at each occurrence is independently hydrogen, halogen, optionally substituted C₁₋₄ alkyl, or optionally substituted C₁₋₄ alkoxy, typically hydrogen or C₁₋₄ alkyl, preferably, the end X groups of the (X)ₘ₁ moiety are not C(═O) or SO₂. In some embodiments, the integer m1 can be 12-50, such as 14-50, 16-30, etc. Preferably, the total number of non-hydrogen atoms of the (X)ₘ₁ moiety is between 15-50, such as 18, 20, 25, 30, 35, 40, 45, or 50, or any ranges between the recited value. In some preferred embodiments, none of the X groups is a ring structure. In some preferred embodiments, one instance of X is a ring structure, preferably 3-10 membered ring structure. For example, in some embodiments, one instance of X is triazole and the remaining X groups are each independently CR₂, —C(R)—C(R)—,

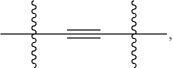 [SMILES: *C#C*]

or O, more preferably, CR₂ or O, wherein R at each occurrence is independently hydrogen or C₁₋₄ alkyl. In some embodiments, none of the X groups is a ring structure, and each X is independently CR₂, —C(R)═C(R)—,

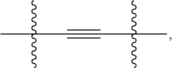 [SMILES: *C#C*]

or O, more preferably, CR₂ or O, wherein R at each occurrence is independently hydrogen or C₁₋₄ alkyl. In some embodiments, up to 6 instances (e.g., 1, 2, 3, 4, 5, or 6) of X can be O. In some embodiments, all instances of X is CR₂, wherein R at each occurrence is independently hydrogen or C₁₋₄ alkyl (preferably, methyl). In some embodiments, one or two instances of X is O, and all other instances of X is CR₂, wherein R at each occurrence is independently hydrogen or C₁₋₄ alkyl (preferably, methyl). In some embodiments, one or two instances of X is O, one X is triazole, and all other instances of X is CR₂, wherein R at each occurrence is independently hydrogen or C₁₋₄ alkyl (preferably, methyl). In some embodiments, R is hydrogen. The definition and preferred definition of G¹, G², G⁴, r, and A⁻ are also described herein, including any of those corresponding definitions shown in compounds according to the compounds listed in Table 1 herein, or any of the compound according to Examples 1-221.

### Formula II and II-B

In some embodiments, the present disclosure provides a compound of Formula II or II-B, or a pharmaceutically acceptable salt or ester thereof:

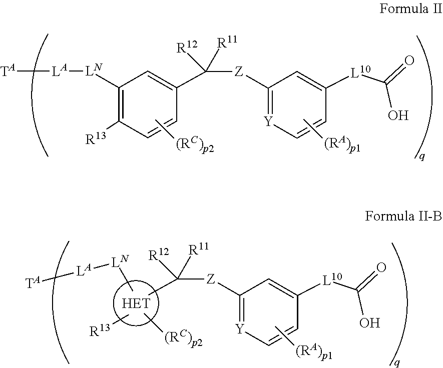

wherein:
Y is CH, CR^{A}, or N;
Z is O, S, NH, or N(C₁₋₄ alkyl);
HET ring stands for an optionally substituted heteroaryl ring (e.g., a 5 or 6-membered heteroaryl, such as a triazole ring);
R¹¹ and R¹² are each independently hydrogen or C₁₋₄ alkyl;
L^{N} is null, an optionally substituted C₁₋₆ alkylene, or an optionally substituted C₁₋₆ heteroalkylene having 1-3 heteroatoms;
L¹⁰ is an alkyelene (e.g., a C₁₋₆ alkyl alkylene), optionally substituted with 1-3 substituents independently selected from halogen, optionally substituted C₁₋₆ alkyl, optionally substituted C₂₋₆ alkenyl, optionally substituted C₂₋₆ alkynyl, optionally substituted C₁₋₆ heteroalkyl, optionally substituted C₃₋₆ cycloalkyl, optionally substituted C₁₋₆ alkoxy, optionally substituted C₃₋₆ cycloalkoxy, optionally substituted heterocyclyl, optionally substituted aryl, and optionally substituted heteroaryl, or two substituents are joined to form an optionally substituted ring structure;
R^{A} at each occurrence is independently halogen, CN, optionally substituted C₁₋₆ alkyl, optionally substituted C₃₋₆ cycloalkyl, optionally substituted C₁₋₆ alkoxy, or optionally substituted C₃₋₆ cycloalkoxy, or two R^{A} are joined to form an optionally substituted ring structure; p1 is 0, 1, or 2;
R^{C} at each occurrence is independently halogen, CN, optionally substituted C₁₋₆ alkyl, optionally substituted C₃₋₆ cycloalkyl, optionally substituted C₁₋₆ alkoxy, or optionally substituted C₃₋₆ cycloalkoxy, or two R^{C} are joined to form an optionally substituted ring structure; p2 is 0, 1, 2, or 3;
R¹³ is hydrogen, an optionally substituted phenyl or an optionally substituted heteroaryl, and
wherein:
q is an integer of 1-10, preferably, 1 or 2, and
T^{A} is a hydrophilic group and L^{A} is a linker. In preferred embodiments,
T^{A} is a hydrophilic group having a terminal atom(s) selected from N, O, S, or C, which is covalently bonded with a first end atom of L^{A}, wherein (1) when the terminal atom(s) is N of a basic amine group, then the corresponding compound T^{A}-(C(O)—CH₃)_{q} has a cLogP of less than 1, wherein the —C(O)—CH₃ is bonded with the terminal N atom(s); (2) when the terminal atom(s) is C of a C(O) group, then the corresponding compound T^{A}-(OH)_{q} has a cLogP of less than 1, wherein the —OH is bonded with the terminal C atom(s); (3) when the terminal atom(s) is S of a SO₂ group, then T^{A}-(OH), has a cLogP of less than 1, wherein the —OH is bonded with the terminal S atom(s); or (4) when (1)-(3) do not apply, then T^{A}-H_{q} has a cLogP of less than 1; and
L^{A} is a linker characterized in that the maximum length between the two end atoms of L^{A} is at least the maximum length between the two end carbon atoms of —(CH₂)₁₀—, wherein (1) when both end atoms of L^{A} are C of a C(O) or S of a SO₂ group, then the corresponding compound HO-L^{A}-OH has a cLogP of at least 3, wherein each —OH is bonded with the end C(O) or SO₂ group; (2) when only one end atom of L^{A} is C of a C(O) or S of a SO₂ group, then the corresponding compound H-L^{A}-OH has a cLogP of at least 3, wherein the —OH is bonded with the end C(O) or SO₂ group; or (3) when neither (1) and (2) applies, then the corresponding compound H-L^{A}-H has a cLogP of at least 3.

In some embodiments, the compound has a structure according to Formula II.

In some embodiments, the compound has a structure according to Formula II-B.

In some embodiments according to Formula II or II-B, L^{N} is null, i.e., L^{A} is directly connected to the phenyl ring drawn in Formula II or the HET ring in II-B. To be clear, for the purposes herein, the definition of L^{N} as null should not be interpreted such that in such embodiments, L^{A} cannot contain a fragment that fits into one or more of the definitions of L^{N} described herein. Rather, in some embodiments, when L^{N} is defined as null, the variable L^{A} can have any of the definitions herein described for L^{N}-L^{A} in embodiments where L^{N} is not null.

In some embodiments according to Formula II or II-B, L^{N} is a branched or straight chained C₁₋₆ alkyl alkylene, such as

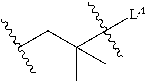

(L^{A} is shown to show direction of connection). In some embodiments according to Formula II or II-B, L^{N} is

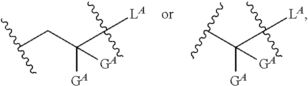

(L^{A} is shown to show direction of connection), wherein G^{A} at each occurrence is independently hydrogen or an optionally substituted C₁₋₄ alkyl, or two G^{A} are joined to form a 3-6 membered ring, such as a cyclopropyl or cyclobutyl ring. In some preferred embodiments, G^{A} at each occurrence is methyl.

In some embodiments according to Formula II or II-B, L^{N} is a branched or straight chained C₁₋₆ alkyl heteroalkylene having one or two oxygen atoms, such as

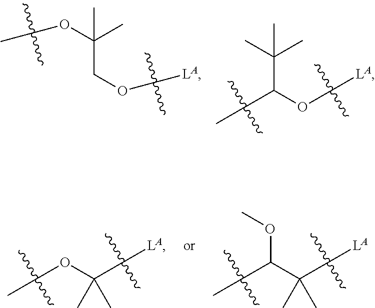

(L^{A} is shown to show direction of connection). In some embodiments according to Formula II or II-B, L^{N} is

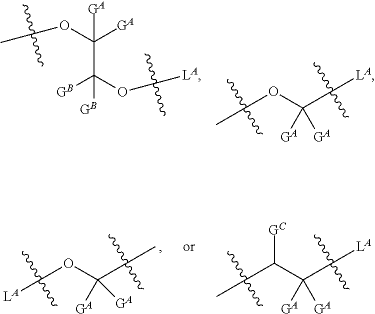

(L^{A} is shown to show direction of connection), wherein G^{A} at each occurrence is independently hydrogen or an optionally substituted C₁₋₄ alkyl, or two G^{A} are joined to form a 3-6 membered ring, such as a cyclopropyl or cyclobutyl ring, wherein G^{B} at each occurrence is independently hydrogen or an optionally substituted C₁₋₄ alkyl, or two G^{B} or one G^{A} and one G^{B} are joined to form a 3-6 membered ring, such as a cyclopropyl or cyclobutyl ring, wherein G^{C} is hydrogen, an optionally substituted C₁₋₄ alkyl, or an optionally substituted C₁₋₄ heteroalkyl (e.g., C₁₋₄ alkoxy). In some preferred embodiments, G^{A} at each occurrence is methyl. In some preferred embodiments, G^{B} at each occurrence is hydrogen. In some preferred embodiments, G^{C} is hydrogen or C₁₋₄ alkoxy such as methoxy.

In some embodiments, q in Formula II is 1, and the compound of Formula II can be characterized as having a Formula II-1:

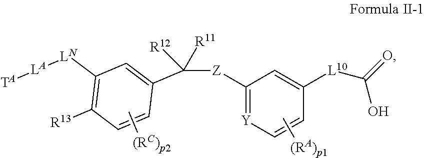

wherein the variables T^{A}, L^{A}, L^{N}, L¹⁰, R¹¹, R¹², R¹³, R^{A}, R^{C}, Y, Z, p1, and p2 are defined herein, which include any of those described and preferred herein in any combinations.

In some embodiments, q in Formula II-B is 1, and the compound of Formula II-B can be characterized as having a Formula II-B-1:

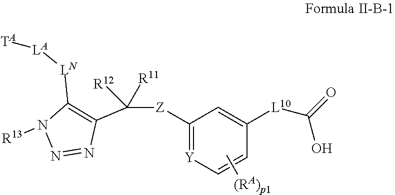

wherein the variables T^{A}, L^{A}, L^{N}, L¹⁰, R¹¹, R¹², R¹³, R^{A}, Y, p1, and Z are defined herein.

### Linker L^{A}

As shown herein, the inventors found that several factors are important for the compound of Formula II or II-B to be a potent GPR40 agonist. One factor that can determine whether the compound of Formula II or II-B can be a potent GPR40 agonist is the length of the linker (e.g., L^{A}) but not the exact chemical structure of the linker. As shown in the Examples section herein, when the length of the linker is below certain threshold, the EC50 value can increase significantly. Accordingly, the present inventors envision that the maximum length between the two end atoms of linker L^{A} in Formula II or II-B should be at least that between the two end carbon atoms of an alkyelene chain —(CH₂)₁₀—, similar to those described hereinabove in connection with Formula I. In other words, the longest chain length of L^{A} should be equal to or greater than the longest chain length of the alkyelene chain-(CH₂)₁₀—. The meaning and determination of the maximum distance between the two end non-hydrogen atoms of linker L^{A} as discussed above for Formula I also apply to Formula II or II-B. In some embodiments, L^{A} can be characterized in that the maximum length between the two end atoms of L^{A} is at least that between the two end carbon atoms of —(CH₂)₁₂—, preferably, at least that of —(CH₂)₁₄—, more preferably, at least that of —(CH₂)₁₆—. In some embodiments, L^{A} can be characterized in that the maximum length between the two end atoms of L^{A} is between (i) the maximum length between the two end carbon atoms of —(CH₂)₁₂— and (ii) the maximum length between the two end carbon atoms of —(CH₂)₅₀—.

In some embodiments, in Formula II (e.g., Formula II-1) or II-B (e.g., Formula II-B-1), L^{A} can be represented by a formula of (X) m, wherein X at each occurrence is independently CR₂, C(═O), —C(R)═C(R)—,

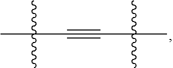 [SMILES: *C#C*]

SiR₂, O, S, SO₂, NR, [NR₂]⁺, or a ring structure, preferably 3-10 membered ring structure, wherein R at each occurrence is independently hydrogen, halogen, optionally substituted C₁₋₄ alkyl, or optionally substituted C₁₋₄ alkoxy, typically hydrogen or C₁₋₄ alkyl, and the integer m is at least 10, such as at least 12, at least 14, at least 16, at least 18, at least 20, at least 50, such as 12-50, 16-50, etc. Suitable X groups for Formula II also include any of those described and preferred herein in connection with Formula I. Typically, in L^{A}, 0, 1, or 2 instances of X representing a ring structure, preferably 3-10 membered ring structure (such as a C₃₋₆ cycloalkyl such as cyclopropyl, a 5 or 6 membered heteroaryl, such as a triazole ring). In some embodiments, two consecutive X can be —C(O)O— or —C(O)NR—. In some embodiments, one instance of X can be a cyclopropane, cyclobutane or bicyclobutane [1.1] ring. In some embodiments, one instance of X can be a 5 or 6-membered heteroaryl, such as a triazole ring. In some embodiments, one instance of X can be a cyclopentane, cyclohexane or cycloheptane ring. Typically, one of the end X groups connects to T^{A} through a carbon atom. The total number of non-hydrogen atoms of L^{A} can be typically between 10-100, such as 12-30, 14-50, 16-50, 18-100, etc.

Another factor that can determine whether the compound of Formula II or II-B can be a potent GPR40 agonist is the hydrophobicity of the linker (e.g., L^{A}) but not the exact chemical structure of the linker. In general, L^{A} should be a hydrophobic moiety. In some embodiments, both end atoms of L^{A} are C of a C(O) or S of a SO₂ group, in such embodiments, the hydrophobicity of L^{A} can be typically characterized in that the corresponding compound HO-L^{A}-OH has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc., wherein each of the —OH is bonded with the end C(O) or SO₂ group. In some embodiments, only one end atom of L^{A} is C of a C(O) or S of a SO₂ group, in such embodiments, the hydrophobicity of L^{A} can be typically characterized in that the corresponding compound H-L^{A}-OH has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc., wherein the —OH is bonded with the end C(O) or SO₂ group. In some embodiments, neither of the end atoms of L^{A} is C of a C(O) or S of a SO₂ group, in such embodiments, in such embodiments, the hydrophobicity of L^{A} can be characterized in that the corresponding compound H-L^{A}-H has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc.

For example, in some embodiments, L^{A} is (X)ₘ₋₁—C(O)—, wherein the C(O) end is bonded with T^{A}, wherein X at each occurrence is independently CR₂, C(═O), —C(R)═C(R)—,

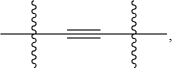 [SMILES: *C#C*]

SiR₂, O, S, SO₂, NR, [NR₂]⁺ or a ring structure, preferably 3-10 membered ring structure, provided that the end X group (i.e., the X group in L^{A} that is furthest away from the C(O) end) is not C(O) or SO₂, wherein R at each occurrence is independently hydrogen, halogen, optionally substituted C₁₋₄ alkyl, or optionally substituted C₁₋₄ alkoxy, typically hydrogen or C₁₋₄ alkyl, and the integer m is at least 10, such as at least 12, at least 14, at least 16, at least 18, at least 20, at least 50, such as 12-50, 16-50, etc., and the hydrophobicity of —(X) m-1—C(O)— is characterized in that the corresponding compound H—(X) m-1-COOH should have a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc. The “m-1” should be understood as the integer m minus 1, not to be misunderstood as a different designated variable.

In some embodiments, L^{A} is (X) m, wherein X at each occurrence is independently CR₂, C(═O), —C(R)═C(R)—,

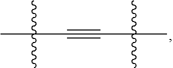 [SMILES: *C#C*]

SiR₂, O, S, SO₂, NR, [NR₂]⁺ or a ring structure, preferably 3-10 membered ring structure, provided that neither of the end X groups is C(O) or SO₂, wherein R at each occurrence is independently hydrogen, halogen, optionally substituted C₁₋₄ alkyl, or optionally substituted C₁₋₄ alkoxy, typically hydrogen or C₁₋₄ alkyl, and the integer m is at least 10, such as at least 12, at least 14, at least 16, at least 18, at least 20, at least 50, such as 12-50, 16-50, etc., then the corresponding compound H—(X)ₘ—H should have a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc.

In some embodiments, L^{A} is -X12-30—(e.g., -X14-, -X16-, -X18-, -X20-, -X24-, -X14-30-, -X16-30-, -X18-30-, etc.), wherein X at each occurrence is independently CR₂, C(═O), —C(R)—C(R)—,

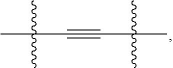 [SMILES: *C#C*]

SiR₂, O, S, SO₂, NR, [NR₂]⁺, or a ring structure, preferably 3-10 membered ring structure, provided that neither of the end X groups is C(O) or SO₂; wherein R at each occurrence is independently hydrogen, halogen, optionally substituted C₁₋₄ alkyl, or optionally substituted C₁₋₄ alkoxy, typically hydrogen or C₁₋₄ alkyl, and wherein the hydrophobicity of L^{A} can be characterized in that the corresponding compound H-L^{A}-H has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc. In some embodiments, the total number of non-hydrogen atoms of L^{A} is between 12-100, such as 12, 14, 16, 18, 20, 24, 30, 40, 50, 100, or any ranges between the recited values, such as 12-30, 14-50, 16-50, 18-100, etc. In some embodiments, two consecutive X can represent-C(O)O— or —C(O) NR—. In some embodiments, one or more (e.g., 1 or 2) instances of X can be a ring structure selected from cyclopropane, cyclobutane, bicyclobutane [1.1], cyclopentane, cyclohexane or cycloheptane. In some embodiments, one or more (e.g., 1) instances of X can be a ring structure selected from phenyl or 5 or 6-membered heteroaryl, such as triazole. In some embodiments, R is hydrogen.

In some embodiments, L^{A} is -X12-30—C(O)—, wherein the C(O) is directly bonded with T^{A}, and wherein X at each occurrence is independently CR₂, C(═O), —C(R)═C(R)—,

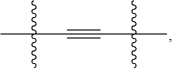 [SMILES: *C#C*]

SiR₂, O, S, SO₂, NR, [NR₂]⁺, or a ring structure, preferably 3-10 membered ring structure, provided that the end X group (i.e., the X group in L^{A} that is furthest away from the C(O) end) is not C(O) or SO₂; wherein R at each occurrence is independently hydrogen, halogen, optionally substituted C₁₋₄ alkyl, or optionally substituted C₁₋₄ alkoxy, typically hydrogen or C₁₋₄ alkyl, and wherein the hydrophobicity of L^{A} can be characterized in that the corresponding compound H-X12-30—C(O)—OH has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc. In some embodiments, the total number of non-hydrogen atoms of L^{A} is between 14-100, such as 14, 16, 18, 20, 24, 30, 40, 50, 100, or any ranges between the recited values, such as 14-30, 14-50, 16-50, 18-100, etc. In some embodiments, two consecutive X can be —C(O)O— or —C(O) NR—. In some embodiments, one or more instances (e.g., 1 or 2) of X can be a ring structure selected from cyclopropane, cyclobutane, bicyclobutane [1.1], cyclopentane, cyclohexane or cycloheptane. In some embodiments, one or more instances (e.g., 1) of X can be a ring structure selected from phenyl or 5 or 6-membered heteroaryl, such as triazole. In some embodiments, R is hydrogen.

In some preferred embodiments, L^{A} is —C₁₂₋₃₀ alkylene- or —C₁₂₋₃₀ alkylene-C(O)—, wherein the —C₁₂₋₃₀ alkylene- is optionally substituted, wherein the optional substituents can optionally be joined together to form a double bond, triple bond, or a ring structure (typically a 3-10 membered ring structure), wherein the end carbon atoms of the —C₁₂₋₃₀ alkylene- are not substituted with oxo (═O), wherein the longest chain length of L^{A} is at least that of —(CH₂)₁₂—, such as at least that of —(CH₂)₁₄—, at least that of (CH₂)₁₆—, at least that of —(CH₂)₁₈—, and wherein the hydrophobicity of L^{A} can be characterized in that the corresponding compound H—C₁₂₋₃₀ alkylene-H or H—C₁₂₋₃₀ alkylene-C(O)—OH has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc. In some embodiments, the total number of non-hydrogen atoms of L^{A} is between 14-100, such as 14, 16, 18, 20, 24, 30, 40, 50, 100, or any ranges between the recited values, such as 14-30, 14-50, 16-50, 18-100, etc. In some embodiments, L^{A} is unsubstituted —C₁₂₋₃₀ alkylene-, such as a linear or branched C₁₂₋₃₀ alkylene. In some embodiments, L^{A} is unsubstituted-C₁₂₋₃₀ alkylene-C(O)—, wherein the C₁₂₋₃₀ alkylene can be linear or branched. In some preferred embodiments, L^{A} is —C₁₂₋₃₀ alkylene- or —C₁₂₋₃₀ alkylene-C(O)—, wherein the C₁₂₋₃₀ alkylene is a linear and unsubstituted-C₁₂₋₃₀ alkylene-.

In some preferred embodiments, L^{A} or L^{N}-L^{A} in Formula II (e.g., Formula II-1) or II-B (e.g., Formula II-B-1) can be selected from a structure of:

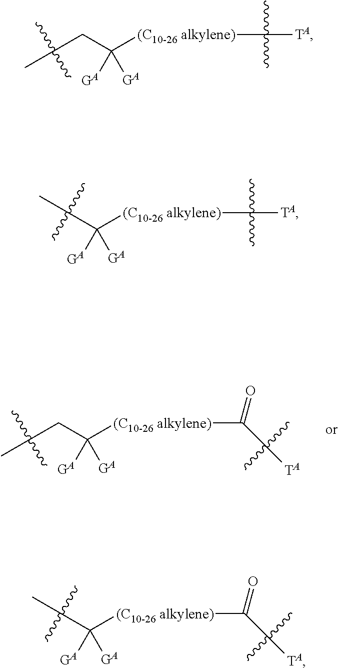

wherein the C₁₀₋₂₆ alkylene is optionally substituted, and wherein G^{A} at each occurrence is independently hydrogen or an optionally substituted C₁₋₄ alkyl, or two G^{A} are joined to form a 3-6 membered ring, such as a cyclopropyl or cyclobutyl ring. In some preferred embodiments, G^{A} at each occurrence is methyl. In some embodiments, the C₁₀₋₂₆ alkylene is unsubstituted, such as a linear or branched C₁₀₋₂₆ alkylene. In some preferred embodiments, the C₁₀₋₂₆ alkylene is a linear and unsubstituted C₁₀₋₂₆ alkylene.

In some embodiments, L^{A} is a 12-30 membered heteroalkylene or -(12-30 membered heteroalkylene)-C(O)—, wherein the 12-30 membered heteroalkylene is optionally substituted and contains 1-6 heteroatoms independently selected from O, N, and S, wherein the sulfur atom(s), if present, is optionally oxidized, wherein the optional substituents can optionally be joined together to form a double bond, triple bond, or a ring structure (typically a 3-10 membered ring structure), wherein the end atoms of the 12-30 membered heteroalkylene are not C of a C(O) or S of a SO₂ group, wherein the longest chain length of L^{A} is at least that of —(CH₂)₁₂—, such as at least that of —(CH₂)₁₄—, at least that of —(CH₂)₁₆—, at least that of —(CH₂)₁₈—, and wherein the hydrophobicity of L^{A} can be characterized in that the corresponding compound H-(12-30 membered heteroalkylene)-H or H-(12-30 membered heteroalkylene)-C(O)—OH has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc. In some embodiments, the total number of non-hydrogen atoms of L^{A} is between 14-100, such as 14, 16, 18, 20, 24, 30, 40, 50, 100, or any ranges between the recited values, such as 14-30, 14-50, 16-50, 18-100, etc. In some embodiments, L^{A} is unsubstituted 12-30 membered heteroalkylene, such as a linear or branched 12-30 membered heteroalkylene. In some embodiments, L^{A} is unsubstituted-(12-30 membered heteroalkylene)-C(O)—, wherein the 12-30 membered heteroalkylene can be linear or branched. In some preferred embodiments, L^{A} is 12-30 membered heteroalkylene or -(12-30 membered heteroalkylene)-C(O)—, wherein the 12-30 membered heteroalkylene is a linear and unsubstituted. In some embodiments, the 12-30 membered heteroalkylene includes 1, 2, 3, 4, or 5 heteroatoms independently selected from O, S, and N.

In some preferred embodiments, L^{A} or L^{N}-L^{A} in Formula II (e.g., Formula II-1) or II-B (e.g., Formula II-B-1) can be selected from a structure of:

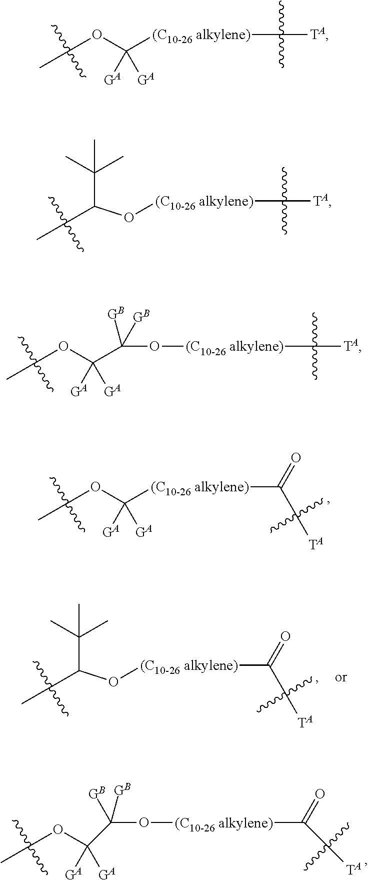

wherein the C₁₀₋₂₆ alkylene is optionally substituted, and wherein G^{A} at each occurrence is independently hydrogen or an optionally substituted C₁₋₄ alkyl, or two G^{A} are joined to form a 3-6 membered ring, such as a cyclopropyl or cyclobutyl ring, wherein G^{B} at each occurrence is independently hydrogen or an optionally substituted C₁₋₄ alkyl, or two G^{B} or one G^{A} and one G^{B} are joined to form a 3-6 membered ring, such as a cyclopropyl or cyclobutyl ring. In some preferred embodiments, G^{A} at each occurrence is methyl. In some preferred embodiments, G^{B} at each occurrence is hydrogen. In some preferred embodiments, L^{A} or L^{N}-L^{A} in Formula II (e.g., Formula II-1) or II-B (e.g., Formula II-B-1) can be selected from a structure of:

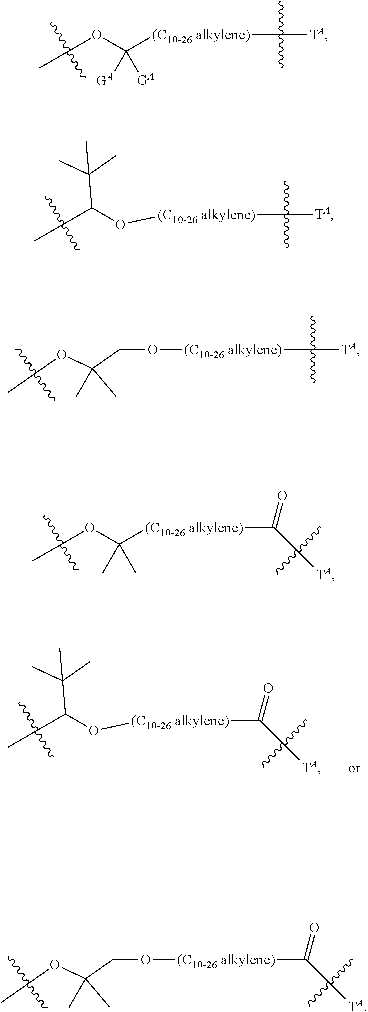

In some embodiments, the C₁₀₋₂₆ alkylene is unsubstituted, such as a linear or branched C₁₀₋₂₆ alkylene. In some preferred embodiments, the C₁₀₋₂₆ alkylene is a linear and unsubstituted C₁₀₋₂₆ alkylene.

In some preferred embodiments, L^{A} can be characterized as having a structure according to

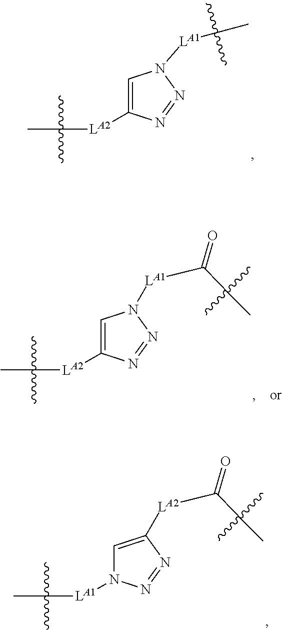

wherein the carbonyl is directly bonded with T^{A}, wherein L^{A1} and L^{A2} are each independently a bond, an optionally substituted —C₁₋₃₀ alkylene-, or an optionally substituted —C₁₋₃₀ heteroalkylene-containing 1-6 heteroatoms independently selected from O, N, and S, wherein the sulfur atom(s), if present, is optionally oxidized, wherein the optional substituents can optionally be joined together to form a double bond, triple bond, or a ring structure, wherein the end atoms of the L^{A1} and L^{A2} that are bonded with T^{A} or L^{N}, as applicable, are not C of a C(O) or S of a SO₂ group, wherein the longest chain length of L^{A} is at least that of —(CH₂)₁₂—, such as at least that of —(CH₂)₁₄—, at least that of (CH₂)₁₆—, at least that of —(CH₂)₁₈—, and wherein the hydrophobicity of L^{A} can be characterized in that the corresponding compound

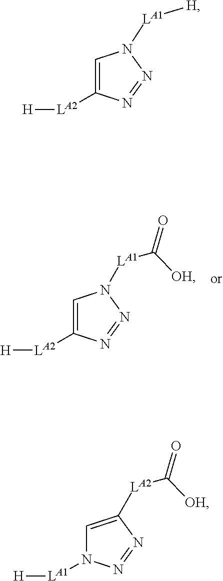

has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc. In some embodiments, the total number of non-hydrogen atoms of L^{A} is between 14-100, such as 14, 16, 18, 20, 24, 30, 40, 50, 100, or any ranges between the recited values, such as 14-30, 14-50, 16-50, 18-100, etc. In some embodiments, L^{A1} is a C₁₋₂₀ alkylene, such as a linear alkylene, (CH₂)₁₋₂₀. In some embodiments, L^{A1} is a C₁₋₁₀ heteroalkylene having 1-3 oxygen atoms, such as —O—(C₁₋₁₀ alkylene)-, etc. In some embodiments, L^{A2} is a bond. In some embodiments, L^{A2} is a C₁₋₂₀ alkylene, such as a linear alkylene, (CH₂)₁₋₂₀. In some embodiments, L^{A2} is a C₁₋₁₀ heteroalkylene having 1-3 oxygen atoms, such as —O—(C₁₋₁₀ alkylene)-, etc.

In some embodiments, L^{A} can be characterized as having a structure according to

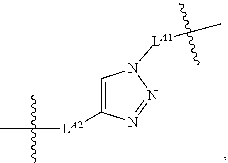

wherein L^{A1} and L^{A2} are each independently a bond, —C₁₋₃₀ alkylene-, or —C₁₋₃₀ heteroalkylene-containing 1-6 heteroatoms independently selected from O, N, and S, wherein the sulfur atom(s), if present, is optionally oxidized, wherein the end atoms of the L^{A1} and L^{A2} that are bonded with T^{A} or L^{N}(it should be understood that when L^{N} is null, then the applicable end atom is bonded directedly to the phenyl ring ortho to R¹³ in Formula II or HET in Formula II-B), as applicable, are not C of a C(O) or S of a SO₂ group, wherein the total number of non-hydrogen atoms of L^{A} is between 15-50, such as 18, 20, 25, 30, 35, 40, 45, or 50, or any ranges between the recited value, wherein the longest chain length of L^{A} is at least that of —(CH₂)₁₂—, such as at least that of —(CH₂)₁₄—, at least that of —(CH₂)₁₆—, at least that of —(CH₂)₁₈—, wherein the hydrophobicity of L^{A} can be characterized in that the corresponding compound H-L^{A}-H has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc. In some embodiments, L^{A1} is a bond (i.e., not present, the triazole nitrogen atom is directly directly bonded with T^{A} or L^{N}). In some embodiments, L^{A2} is a bond. In some embodiments, L^{A1} is a C₁₋₂₀ alkylene, such as a linear alkylene, (CH₂)₁₋₂₀. In some embodiments, L^{A1} is a C₁₋₁₀ heteroalkylene having 1-3 oxygen atoms, such as —O—(C₁₋₁₀ alkylene)-, etc. In some embodiments, L^{A2} is a C₁₋₂₀ alkylene, such as a linear alkylene, (CH₂)₁₋₂₀. In some embodiments, L^{A2} is a C₁₋₁₀ heteroalkylene having 1-3 oxygen atoms, such as —O—(C₁₋₁₀ alkylene)-, etc.

In some embodiments, L^{A} can be characterized as having a structure according to

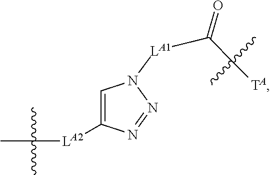

wherein L^{A1} and L^{A2} are each independently a bond, —C₁₋₃₀ alkylene-, or —C₁₋₃₀ heteroalkylene-containing 1-6 heteroatoms independently selected from O, N, and S, wherein the sulfur atom(s), if present, is optionally oxidized, wherein the end atom of the L^{A2} that is bonded with L^{N} is not C of a C(O) or S of a SO₂ group, wherein the total number of non-hydrogen atoms of L^{A} is between 15-50, such as 18, 20, 25, 30, 35, 40, 45, or 50, or any ranges between the recited value, wherein the longest chain length of L^{A} is at least that of —(CH₂)₁₂—, such as at least that of —(CH₂)₁₄—, at least that of —(CH₂)₁₆—, at least that of —(CH₂)₁₈—, wherein the hydrophobicity of L^{A} can be characterized in that the corresponding compound

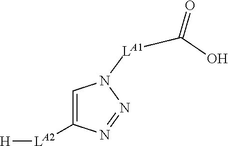

has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc. In some embodiments, L^{A1} is a bond. In some embodiments, L^{A2} is a bond. In some embodiments, L^{A1} is a C₁₋₂₀ alkylene, such as a linear alkylene, (CH₂)₁₋₂₀. In some embodiments, L^{A1} is a C₁₋₁₀ heteroalkylene having 1-3 oxygen atoms, such as —O—(C₁₋₁₀ alkylene)-, etc. In some embodiments, L^{A2} is a C₁₋₂₀ alkylene, such as a linear alkylene, (CH₂)₁₋₂₀. In some embodiments, L^{A2} is a C₁₋₁₀ heteroalkylene having 1-3 oxygen atoms, such as —O—(C₁₋₁₀ alkylene)-, etc.

In some embodiments, L^{A} can be characterized as having a structure according to

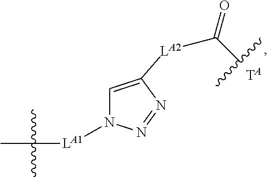

wherein L^{A1} and L^{A2} are each independently a bond, —C₁₋₃₀ alkylene-, or —C₁₋₃₀ heteroalkylene-containing 1-6 heteroatoms independently selected from O, N, and S, wherein the sulfur atom(s), if present, is optionally oxidized, wherein the end groups of the L^{A1} that is bonded with L^{N} is not C of a C(O) or S of a SO₂ group, wherein the total number of non-hydrogen atoms of L^{A} is between 15-50, such as 18, 20, 25, 30, 35, 40, 45, or 50, or any ranges between the recited value, wherein the longest chain length of L^{A} is at least that of —(CH₂)₁₂—, such as at least that of —(CH₂)₁₄—, at least that of —(CH₂)₁₆—, at least that of —(CH₂)₁₈—, wherein the hydrophobicity of L^{A} can be characterized in that the corresponding compound

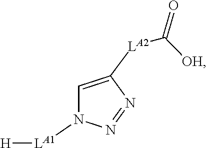

has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc. In some embodiments, L^{A1} is a bond. In some embodiments, L^{A2} is a bond. In some embodiments, L^{A1} is a C₁₋₂₀ alkylene, such as a linear alkylene, (CH₂)₁₋₂₀. In some embodiments, L^{A1} is a C₁₋₁₀ heteroalkylene having 1-3 oxygen atoms, such as —O—(C₁₋₁₀ alkylene)-, etc. In some embodiments, L^{A2} is a C₁₋₂₀ alkylene, such as a linear alkylene, (CH₂)₁₋₂₀. In some embodiments, L^{A2} is a C₁₋₁₀ heteroalkylene having 1-3 oxygen atoms, such as —O—(C₁₋₁₀ alkylene)-, etc.

In some preferred embodiments, L^{A} or L^{N}-L^{A} in Formula II (e.g., Formula II-1) or II-B (e.g., Formula II-B-1) can be selected from a structure of:

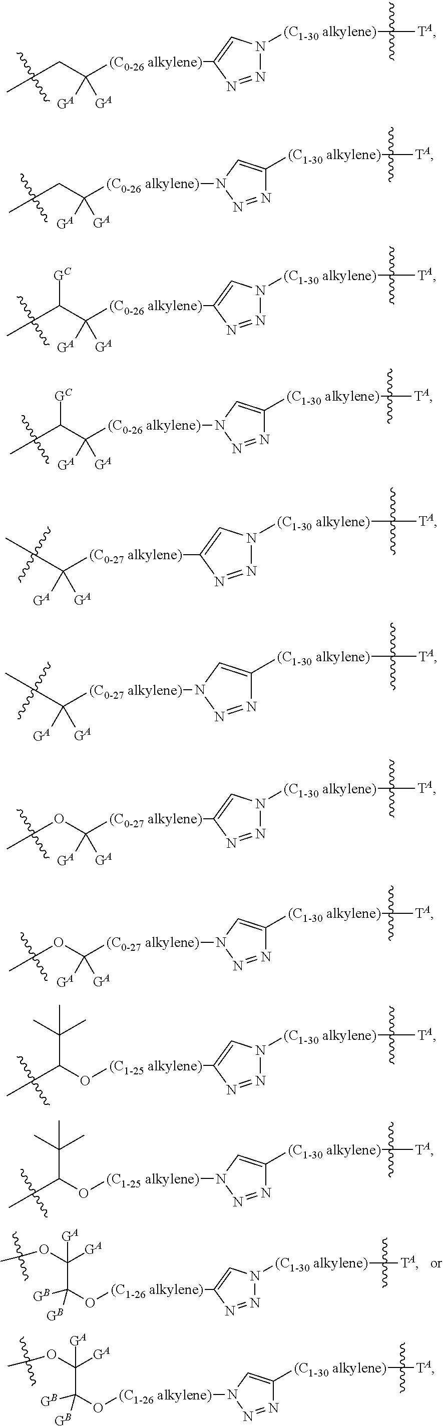

wherein the C₀₋₂₆ alkylene, C₀₋₂₇ alkylene, C₁₋₂₅ alkylene, C₁₋₂₆ alkylene, or C₁₋₃₀ alkylene is optionally substituted, and wherein G^{A} at each occurrence is independently hydrogen or an optionally substituted C₁₋₄ alkyl, or two G^{A} are joined to form a 3-6 membered ring, such as a cyclopropyl or cyclobutyl ring, wherein G^{B} at each occurrence is independently hydrogen or an optionally substituted C₁₋₄ alkyl, or two G^{B} or one G^{A} and one G^{B} are joined to form a 3-6 membered ring, such as a cyclopropyl or cyclobutyl ring, wherein G^{C} is hydrogen, an optionally substituted C₁₋₄ alkyl, or an optionally substituted C₁₋₄ heteroalkyl (e.g., C₁₋₄ alkoxy). In some preferred embodiments, G^{A} at each occurrence is methyl. In some preferred embodiments, G^{B} at each occurrence is hydrogen. In some embodiments, the C₀₋₂₆ alkylene, C₀₋₂₇ alkylene, C₁₋₂₅ alkylene, C₁₋₂₆ alkylene, or C₁₋₃₀ alkylene is unsubstituted, which is a linear or branched alkylene. In some embodiments, the C₀₋₂₆ alkylene, C₀₋₂₇ alkylene, C₁₋₂₅ alkylene, C₁₋₂₆ alkylene, or C₁₋₃₀ alkylene is linear and unsubstituted alkylene. To be clear, a Co alkylene used herein should be understood as non-existent. For example, in the structure of

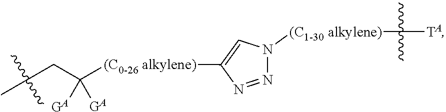

when the C₀₋₂₆ alkylene is a C₀ alkylene, the structure should be understood as

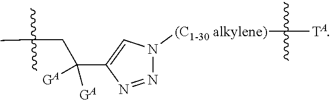

In some preferred embodiments, L^{A} or L^{N}-L^{A} in Formula II (e.g., Formula II-1) or II-B (e.g., Formula II-B-1) can be selected from a structure of:

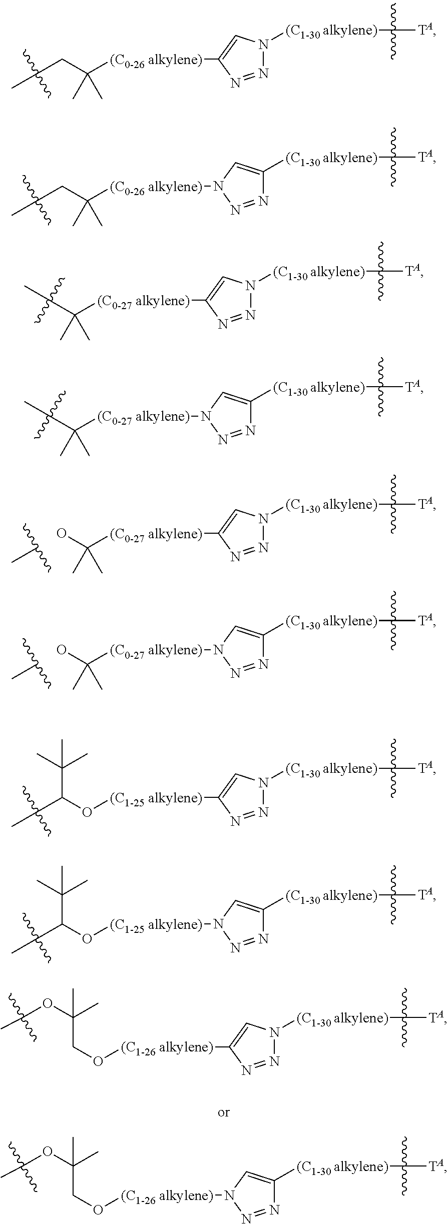

wherein the C₀₋₂₆ alkylene, C₀₋₂₇ alkylene, C₁₋₂₅ alkylene, C₁₋₂₆ alkylene, or C₁₋₃₀ alkylene is optionally substituted. In some embodiments, the C₀₋₂₆ alkylene, C₀₋₂₇ alkylene, C₁₋₂₅ alkylene, C₁₋₂₆ alkylene, or C₁₋₃₀ alkylene is unsubstituted, which is a linear or branched alkylene. In some embodiments, the C₀₋₂₆ alkylene, C₀₋₂₇ alkylene, C₁₋₂₅ alkylene, C₁₋₂₆ alkylene, or C₁₋₃₀ alkylene is linear and unsubstituted alkylene.

In some preferred embodiments, L^{A} or L^{N}-L^{A} in Formula II (e.g., Formula II-1) or II-B (e.g., Formula II-B-1) can be selected from a structure of:

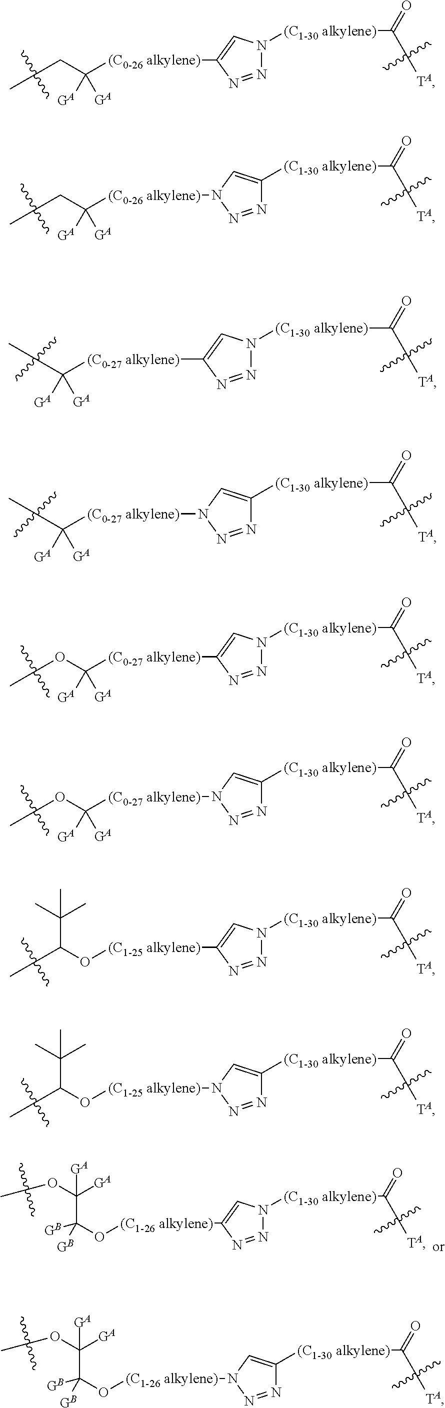

wherein the C₀₋₂₆ alkylene, C₀₋₂₇ alkylene, C₁₋₂₅ alkylene, C₁₋₂₆ alkylene, or C₁₋₃₀ alkylene is optionally substituted, and wherein G^{A} at each occurrence is independently hydrogen or an optionally substituted C₁₋₄ alkyl, or two G^{A} are joined to form a 3-6 membered ring, such as a cyclopropyl or cyclobutyl ring, wherein G^{B} at each occurrence is independently hydrogen or an optionally substituted C₁₋₄ alkyl, or two G^{B} or one G^{A} and one G^{B} are joined to form a 3-6 membered ring, such as a cyclopropyl or cyclobutyl ring. In some preferred embodiments, G^{A} at each occurrence is methyl. In some preferred embodiments, G^{B} at each occurrence is hydrogen. In some embodiments, the C₀₋₂₆ alkylene, C₀₋₂₇ alkylene, C₁₋₂₅ alkylene, C₁₋₂₆ alkylene, or C₁₋₃₀ alkylene is unsubstituted, which is a linear or branched alkylene. In some embodiments, the C₀₋₂₆ alkylene, C₀₋₂₇ alkylene, C₁₋₂₅ alkylene, C₁₋₂₆ alkylene, or C₁-30 alkylene is linear and unsubstituted alkylene.

In some preferred embodiments, L^{A} or L^{N}-L^{A} in Formula II (e.g., Formula II-1) or II-B (e.g., Formula II-B-1) can be selected from a structure of:

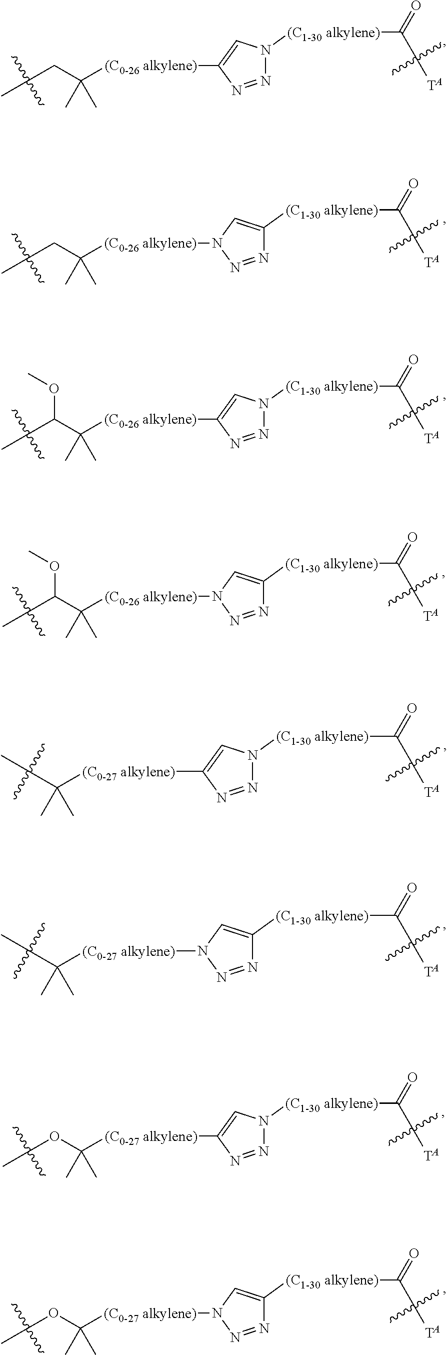

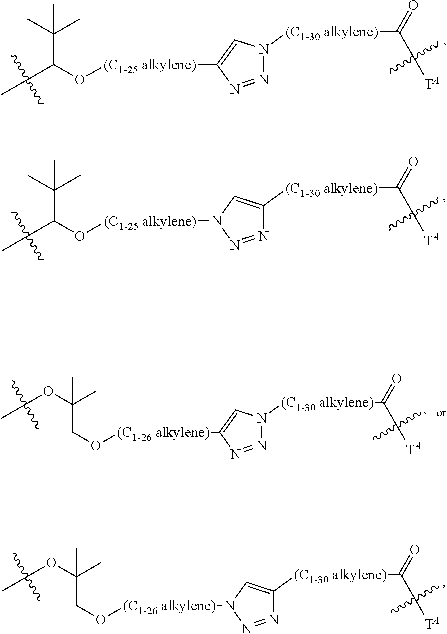

wherein the C₀₋₂₆ alkylene, C₀₋₂₇ alkylene, C₁₋₂₅ alkylene, C₁₋₂₆ alkylene, or C₁₋₃₀ alkylene is optionally substituted. In some embodiments, the C₀₋₂₆ alkylene, C₀₋₂₇ alkylene, C₁₋₂₅ alkylene, C₁₋₂₆ alkylene, or C₁₋₃₀ alkylene is unsubstituted, which is a linear or branched alkylene. In some embodiments, the C₀₋₂₆ alkylene, C₀₋₂₇ alkylene, C₁₋₂₅ alkylene, C₁₋₂₆ alkylene, or C₁₋₃₀ alkylene is linear and unsubstituted alkylene.

### Hydrophilic Group T^{A}

As with compounds of Formula I, another factor that can determine whether the compound of Formula II or II-B can be a potent GPR40 agonist is the hydrophilicity or polarity of T^{A}, but the exact chemical structure of T^{A} is not as important. Suitable and preferred T^{A} for Formula II or II-B include any of those described and preferred in connection with Formula I. In Formula II or II-B, T^{A} is a hydrophilic group having a terminal atom(s) selected from N, O, S, or C, which is covalently bonded with an end atom such as carbon or nitrogen atom(s) of L^{A}, wherein (1) when the terminal atom(s) is N of a basic amine group, then the corresponding compound T^{A}-(C(O)—CH₃)_{q} has a cLogP of less than 1, wherein the —C(O)—CH₃ is bonded with the terminal N atom(s); (2) when the terminal atom(s) is C of a C(O) group, then the corresponding compound T^{A}-(OH)_{q} has a cLogP of less than 1, wherein the —OH is bonded with the terminal C atom(s); (3) when the terminal atom(s) is S of a SO₂ group, then the corresponding compound T^{A}-(OH)_{q} has a cLogP of less than 1, wherein the —OH is bonded with the terminal S atom(s); or (4) when (1)-(3) do not apply, then T^{A}-H_{q} has a cLogP of less than 1. In some embodiments, the terminal atom(s) is N of a basic amine group, and T^{A} is characterized in that the corresponding compound T^{A}-(C(O)—CH₃)_{q} has a cLogP of less than 0 (e.g., less than −1, less than −2, less than −3, less than-3.5, less than −4, or even lower). In some embodiments, the terminal atom(s) is C of a C(O) group, and T^{A} is characterized in that the corresponding compound T^{A}-(OH)_{q} has a cLogP of less than 0 (e.g., less than −1, less than −2, less than −3, less than −3.5, less than −4, or even lower). In some embodiments, the terminal atom(s) is S in a SO₂ group, and T^{A} is characterized in that the corresponding compound T^{A}-(OH), has a cLogP of less than 0 (e.g., less than −1, less than −2, less than −3, less than −3.5, less than −4, or even lower). In some embodiments, the terminal atom(s) is not N of a basic amine group, C of a C(O) group, or S in a SO₂ group, and T^{A} is characterized in that the corresponding compound T^{A}-H_{q} has a cLogP of less than 0 (e.g., less than −1, less than −2, less than −3, less than −3.5, less than −4, or even lower).

In some preferred embodiments, T^{A} can contain a charged group, including positively charged, such as containing a quaternary nitrogen atom, negatively charged, such as SO₃⁻, or containing a zwitterion structure. When T^{A} contains a charged group, it should be understood that a counterion, preferably, a pharmaceutically acceptable anion or cation, if necessary, exists to balance the charges so that the compound of Formula II or II-B is overall neutral.

In some embodiments, T^{A} is characterized as having a charged group (including zwitterion structures) or a group that can become charged at pH 7, such as primary amine, secondary amine, tertiary amine, quaternary amine, carboxylic acid, etc.

In some embodiments, T^{A} is characterized as having at least two hydrogen bond donors.

In some embodiments, T^{A} is characterized as having at least two hydrogen bond acceptors.

In some embodiments, T^{A} is T^{A1}, T^{A1}-L^{B}-, T^{A1}-L^{B}-(heteroalkylene), or T^{A1}-L^{B}-(heteroalkylene)-L^{B}-, wherein L^{B} at each occurrence is independently —SO₂—, —C(═O)—, or a moiety selected from:

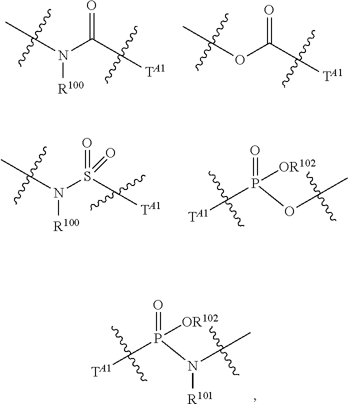

wherein R¹⁰⁰, R¹⁰¹ and R¹⁰² at each occurrence is independently hydrogen, optionally substituted alkyl, or optionally substituted cycloalkyl, wherein T^{A1} is characterized as having a structure of:

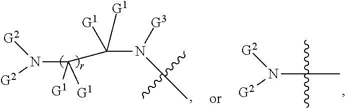

wherein G¹ at each occurrence is independently hydrogen, halogen, optionally substituted C₁₋₄ alkyl, or optionally substituted C₁₋₄ heteroalkyl (such as optionally substituted C₁₋₄ alkoxy), or two gem G¹ group can join to form an ═O, ═NH, or ═N(C₁₋₄ alkyl), or two or more G¹ groups can join to form a ring structure,
the integer r is 1-10, such as 1 or 2,
G² at each occurrence is independently hydrogen, G¹⁰, C(O)-G¹⁰, SO₂G¹⁰, C(O)—NH-G¹⁰, or SO₂NHG¹⁰, C(O)—O-G¹⁰, or SO₂OG¹⁰, wherein G¹⁰ at each occurrence is independently an optionally substituted alkyl, an optionally substituted heteroalkyl, or an optionally substituted 3-10 membered ring structure, which can be carbocyclic, heterocyclic, aromatic, heteroaromatic, or a combination thereof,
G³ is hydrogen or an optionally substituted alkyl (e.g., C₁₋₄ alkyl);
or two G² or one G² and one G¹ or one G¹ and G³ can be joined to form a ring structure such as a 3-10 membered ring structure,
wherein the fragment

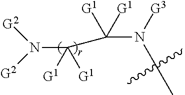

is further characterized in that (i) when the CG¹G¹ group next to the NG³ is not C(═O), the corresponding compound

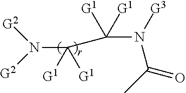

has a cLogP less than 1, preferably, less than 0 (e.g., less than −1, less than −2, less than −3, less than −3.5, less than −4, or even lower), or (ii) when the CG¹G¹ group next to the NG³ is C(═O), the corresponding compound

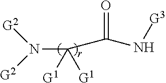

has a cLogP less than 1, preferably, less than 0 (e.g., less than −1, less than −2, less than −3, less than −3.5, less than −4, or even lower), and
wherein the fragment

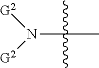

is further characterized in that (i) when the nitrogen is a basic nitrogen, the corresponding compound (G²)₂N—C(O)—CH₃ has a cLogP less than 1, preferably, less than 0 (e.g., less than −1, less than −2, less than −3, less than −3.5, less than −4, or even lower), or (ii) when the nitrogen is a nonbasic nitrogen, the corresponding compound (G²)₂N—H has a cLogP less than 1, preferably, less than 0 (e.g., less than −1, less than −2, less than −3, less than −3.5, less than −4, or even lower). In preferred embodiments, G³ is hydrogen. In some preferred embodiments, T^{A} is T^{A1} or T^{A1}-L^{B}-. In some embodiments, T^{A} is T^{A1}-L^{B}-(heteroalkylene) or T^{A1}-L^{B}-(heteroalkylene)-L^{B}-, wherein the heteroalkylene can be a glycol chain, such as a polyethylene glycol chain.

In some embodiments, T^{A} is T^{A1}. In some embodiments, T^{A} is T^{A1}—C(═O)—. In some embodiments, T^{A1} is characterized as having a structure of:

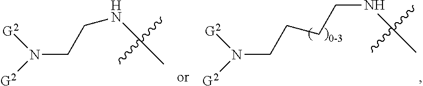

In some embodiments, T^{A1} is characterized as having a structure of:

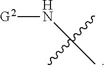

In some embodiments, G² at each occurrence independently can have a structure of

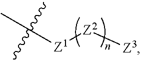

wherein n is an integer of 0-100, preferably, 0-10 (e.g., 0-4, 1-5 or 2-6 etc.), Z¹ is CH₂, C(O), SO, SO₂, CH(C₁₋₄ alkyl), C(C₁₋₄ alkyl)(C₁₋₄ alkyl) or 3-10 membered ring structure, Z² at each occurrence is independently CH₂, O, S, SO, SO₂, C(O), CH(C₁₋₄ alkyl), C(C₁₋₄ alkyl)(C₁₋₄ alkyl), NH, N(C₁₋₄ alkyl), [N(C₁₋₄ alkyl)(C₁₋₄ alkyl)]⁺ or 3-10 membered ring structure, and Z³ is H, C₁₋₄ alkyl, OH, SO₃H, COOH, NH₂, NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, SO₂NH₂, CONH(C₁₋₄ alkyl), CON(C₁₋₄ alkyl)(C₁₋₄ alkyl), SO₂NH(C₁₋₄ alkyl), or SO₂N(C₁₋₄ alkyl)(C₁₋₄ alkyl), wherein each of the C₁₋₄ alkyl is independently selected and optionally substituted, for example, with 1-3 G²⁰ groups defined herein. In some embodiments, none of the Z² is a 3-10 membered ring structure. In some embodiments, one or more instances of Z² is a 3-10 membered ring structure, such as a nitrogen containing ring, for example,

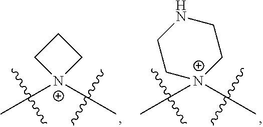 [SMILES: *[N+]1(*)CCC1 ; *[N+]1(*)CCNCC1]

etc. In some embodiments, the Z² directly connects to Z³ is a 3-10 membered ring structure, such as a nitrogen containing ring, for example,

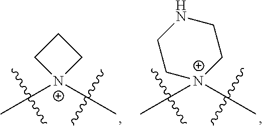 [SMILES: *[N+]1(*)CCC1 ; *[N+]1(*)CCNCC1]

etc. Preferably, at most 2 instances of Z² is a 3-10 membered ring structure. In some embodiments, G² at each occurrence is independently hydrogen, an optionally substituted C₁₋₆ alkyl or an optionally substituted C₁₋₆ heteroalkyl, for example, when substituted, the C₁₋₆ alkyl can be substituted with 1-3 substituents independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), and SO₂N(C₁₋₄ alkyl)(C1-4 alkyl), wherein each of the C₁₋₄ alkyl is independently selected and optionally substituted, for example, with 1-3 G²⁰ groups defined herein. The G²⁰ group herein at each occurrence can be independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), SO₂N(C₁₋₄ alkyl)(C₁₋₄ alkyl), or 3-10 membered ring structure, wherein each of the C₁₋₄ alkyl is independently selected and optionally substituted, for example, with 1-3 G²¹ groups defined herein. The G²¹ group herein at each occurrence can be independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), SO₂N(C₁₋₄ alkyl)(C₁₋₄ alkyl) or 3-10 membered ring structure. In some embodiments, one or more instances of G² is C₁₋₄ alkyl, optionally substituted with COOH or an amide derivative, such as CONH—(C₁₋₄ alkylene)-NH₂ or CONH—(C₁₋₄ alkylene)-[N(C₁₋₄ alkyl)(C₁₋₄ alkyl)(C₁₋₄ alkyl)]⁺. For example, in some embodiments, one or two instances of G² is —(C₁₋₄ alkylene)-COOH, such as —CH₂COOH, and any remaining instance(s) of G² is C₁₋₄ alkyl. In some embodiments, one or two instances of G² is —(C₁₋₄ alkylene)-CONH—(C₁₋₄ alkylene)-NH₂, such as

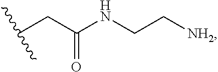 [SMILES: *CC(=O)NCCN]

and any remaining instance(s) of G² is C₁₋₄ alkyl. In some embodiments, one or two instances of G² is —(C₁₋₄ alkylene)-CONH—(C₁₋₄ alkylene)-[N(C₁₋₄ alkyl)(C₁₋₄ alkyl)(C₁₋₄ alkyl)]⁺, such as

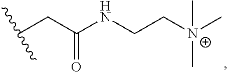 [SMILES: *CC(=O)NCC[N+](C)(C)C]

and any remaining instance(s) of G² is C₁₋₄ alkyl. In some embodiments, T^{A1} can be

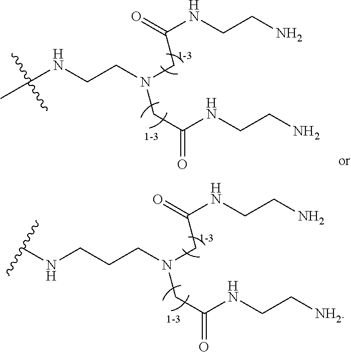

In some embodiments, T^{A1} can be

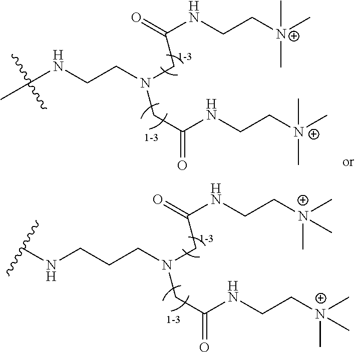

charge balanced with a counterion (e.g., described herein) as necessary. In some embodiments, T^{A1} can be

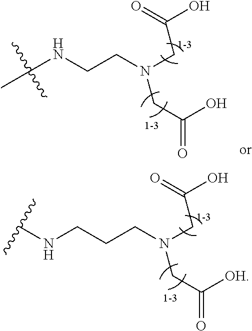

In some embodiments, T^{A1} can be

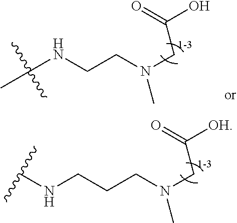

In some embodiments, T^{A1} can be

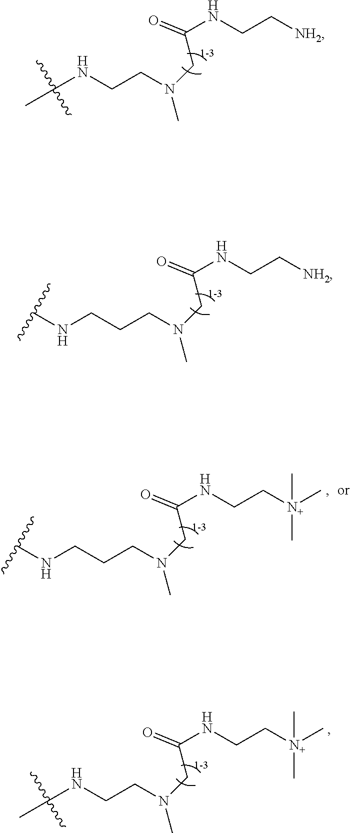

charge balanced with a counterion (e.g., described herein) as necessary. In some embodiments, T^{A1} can be

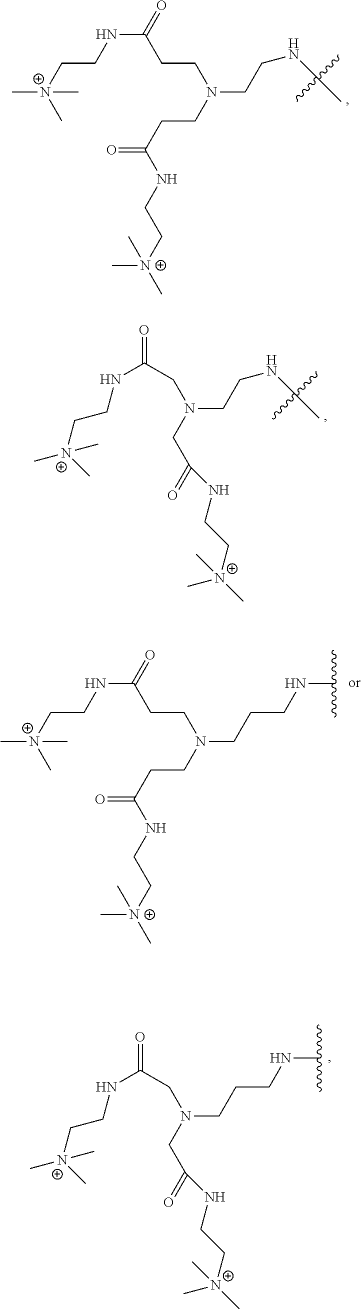 [SMILES: *NCCN(CCC(=O)NCC[N+](C)(C)C)CCC(=O)NCC[N+](C)(C)C ; *NCCN(CC(=O)NCC[N+](C)(C)C)CC(=O)NCC[N+](C)(C)C ; *NCCCN(CCC(=O)NCC[N+](C)(C)C)CCC(=O)NCC[N+](C)(C)C ; *NCCCN(CC(=O)NCC[N+](C)(C)C)CC(=O)NCC[N+](C)(C)C]

charge balanced with a counterion (e.g., described herein) as necessary. For example, in some embodiments, T^{A1} can be

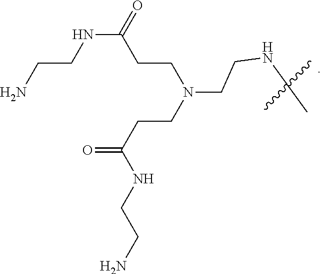 [SMILES: *NCCN(CCC(=O)NCCN)CCC(=O)NCCN]

In some embodiments, T^{A1} can be

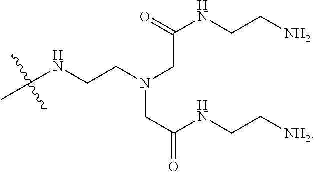 [SMILES: *NCCN(CC(=O)NCCN)CC(=O)NCCN]

In some embodiments, T^{A1} can be

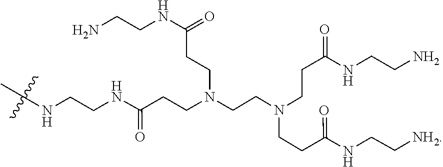 [SMILES: *NCCNC(=O)CCN(CCC(=O)NCCN)CCN(CCC(=O)NCCN)CCC(=O)NCCN]

In some embodiments, T^{A1} can be

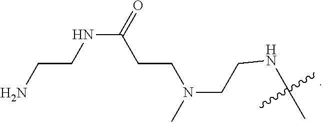 [SMILES: *NCCN(C)CCC(=O)NCCN]

In some embodiments, T^{A1} can be

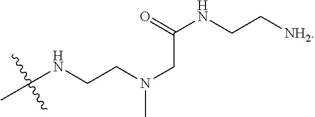 [SMILES: *NCCN(C)CC(=O)NCCN]

In some embodiments, T^{A1} can be

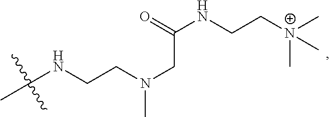 [SMILES: *NCCN(C)CC(=O)NCC[N+](C)(C)C]

charge balanced with a counterion (e.g., described herein) as necessary. In some embodiments, T^{A1} can be

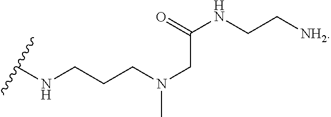 [SMILES: *NCCCN(C)CC(=O)NCCN]

In some embodiments, T^{A1} can be

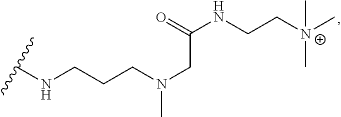 [SMILES: *NCCCN(C)CC(=O)NCC[N+](C)(C)C]

charge balanced with a counterion (e.g., described herein) as necessary. In some embodiments, T^{A1} can be

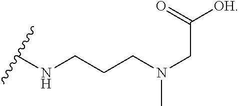 [SMILES: *NCCCN(C)CC(=O)O]

In some embodiments, T^{A} is T^{A1}, T^{A1}-L^{B}-, T^{A1}-L^{B}-(heteroalkylene), or T^{A1}-L^{B}-(heteroalkylene)-L^{B}-, wherein L^{B} at each occurrence is independently —SO₂—, —C(═O)—, or a moiety selected from:

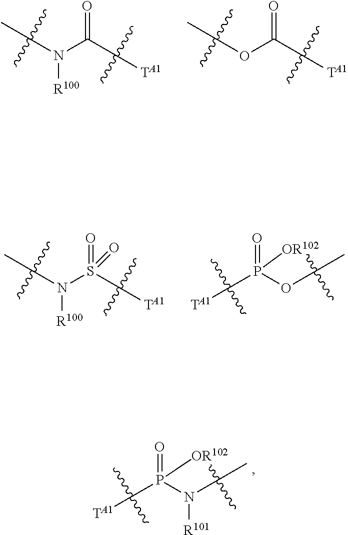

wherein R¹⁰⁰, R¹⁰¹ and R¹⁰² at each occurrence is independently hydrogen, optionally substituted alkyl, or optionally substituted cycloalkyl, wherein T^{A1} is characterized as having a structure of:

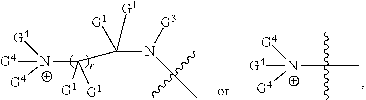

charge balanced with a counterion (e.g., described herein) as necessary, wherein G¹ at each occurrence is independently hydrogen, halogen, optionally substituted C₁₋₄ alkyl, or optionally substituted C₁₋₄ heteroalkyl (such as optionally substituted C₁₋₄ alkoxy), or two gem G¹ group can join to form an ═O, ═NH, or ═N(C₁₋₄ alkyl), or two or more G¹ groups can join to form a ring structure,
the integer r is 1-10, such as 1 or 2,
G³ is hydrogen or an optionally substituted alkyl (e.g., C₁₋₄ alkyl);
G⁴ at each occurrence is independently hydrogen or G¹⁰, wherein G¹⁰ at each occurrence is independently an optionally substituted alkyl, an optionally substituted heteroalkyl, or an optionally substituted 3-10 membered ring structure, which can be carbocyclic, heterocyclic, aromatic, heteroaromatic, or a combination thereof, or two G⁴ or one G⁴ and one G¹ or one G¹ and G³ can be joined to form an optionally substituted 3-10 membered ring structure,
wherein the fragment

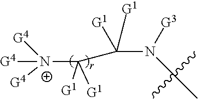

is characterized in that (i) when the CG¹G¹ group next to the NH is not C(═O), the corresponding compound

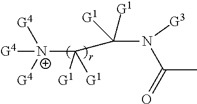

has a cLogP less than 1, preferably, less than 0 (e.g., less than −1, less than −2, less than −3, less than −3.5, less than −4, or even lower), or (ii) when the CG′G′ group next to the NH is C(═O), the corresponding compound

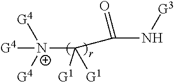

has a cLogP less than 1, preferably, less than 0 (e.g., less than −1, less than −2, less than −3, less than −3.5, less than −4, or even lower). In preferred embodiments, G³ is hydrogen. In some preferred embodiments, T^{A} is T^{A1} or T^{A1}-L^{B}-. In some embodiments, T^{A} is T^{A1}-L^{B}- (heteroalkylene) or T^{A1}-L^{B}- (heteroalkylene)-L^{B}-, wherein the heteroalkylene can be a glycol chain, such as a polyethylene glycol chain. In some embodiments, T^{A} is T^{A1}. In some embodiments, T^{A} is T^{A1}—C(═O)—. In some embodiments, T^{A1} is characterized as having a structure of:

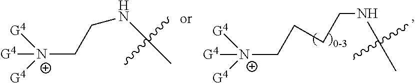

charge balanced with a counterion (e.g., described herein) as necessary. In some embodiments, T^{A1} is characterized as having a structure of:

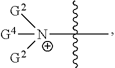

charge balanced with a counterion (e.g., described herein) as necessary. In some embodiments, G⁴ at each occurrence independently can have a structure of

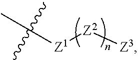

wherein n is an integer of 0-100, preferably, 0-10 (e.g., 0-4, 1-5 or 2-6 etc.), Z¹ is CH₂, C(O), SO₂, CH(C₁₋₄ alkyl), or C(C₁₋₄ alkyl)(C₁₋₄ alkyl), Z² at each occurrence is independently CH₂, O, S, SO, SO₂, C(O), CH(C₁₋₄ alkyl), C(C₁₋₄ alkyl)(C₁₋₄ alkyl), NH, N(C₁₋₄ alkyl), [N(C₁₋₄ alkyl)(C₁₋₄ alkyl)]⁺ or 3-10 membered ring structure, and Z³ is H, C₁₋₄ alkyl, OH, SO₃H, COOH, NH₂, NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, SO₂NH₂, CONH(C₁₋₄ alkyl), CON(C₁₋₄ alkyl)(C₁₋₄ alkyl), SO₂NH(C₁₋₄ alkyl), or SO₂N(C₁₋₄ alkyl)(C₁₋₄ alkyl), wherein each of the C₁₋₄ alkyl is independently selected and optionally substituted, for example, with 1-3 G²⁰ groups defined herein. Preferably, at most 2 instances of Z² is a 3-10 membered ring structure. In some embodiments, none of the Z² is a 3-10 membered ring structure. In some embodiments, one instance of Z² is a 3-10 membered ring structure, such as a nitrogen containing ring, for example,

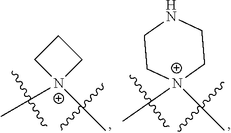 [SMILES: *[N+]1(*)CCC1 ; *[N+]1(*)CCNCC1]

etc. In some embodiments, G⁴ at each occurrence is independently hydrogen, an optionally substituted C₁₋₆ alkyl or an optionally substituted C₁₋₆ heteroalkyl, for example, when substituted, the C₁₋₆ alkyl can be substituted with 1-3 substituents independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), and SO₂N(C₁₋₄ alkyl)(C1-4 alkyl), wherein each of the C₁₋₄ alkyl is independently selected and optionally substituted, for example, with 1-3 G²⁰ groups defined herein. The G²⁰ group herein at each occurrence can be independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), SO₂N(C₁₋₄ alkyl)(C₁₋₄ alkyl), or 3-10 membered ring structure, wherein each of the C₁₋₄ alkyl is independently selected and optionally substituted, for example, with 1-3 G²¹ groups defined herein. The G²¹ group herein at each occurrence can be independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ one or more instances of G⁴ is C₁₋₄ alkyl, such as methyl. In some embodiments, one or more instances of G⁴ is C₁₋₄ alkyl, optionally substituted with COOH or an amide derivative, such as CONH—(C₁₋₄ alkylene)-NH₂ or CONH—(C₁₋₄ alkylene)-[N(C₁₋₄ alkyl)(C₁₋₄ alkyl)(C₁₋₄ alkyl)]⁺. For example, in some embodiments, one or two instances of G⁴ is —(C₁₋₄ alkylene)-COOH, such as —CH₂COOH, and any remaining instance(s) of G⁴ is C₁₋₄ alkyl. In some embodiments, one or two instances of G⁴ is —(C₁₋₄ alkylene)-CONH—(C₁₋₄ alkylene)-NH₂, such as

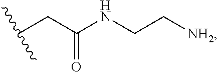 [SMILES: *CC(=O)NCCN]

and any remaining instance(s) of G⁴ is C₁₋₄ alkyl. In some embodiments, one or two instances of G⁴ is —(C₁₋₄ alkylene)-CONH—(C₁₋₄ alkylene)-[N(C₁₋₄ alkyl)(C₁₋₄ alkyl)(C₁₋₄ alkyl)]⁺, such as

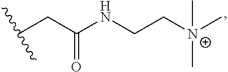 [SMILES: *CC(=O)NCC[N+](C)(C)C]

and any remaining instance(s) of G⁴ is C₁₋₄ alkyl. In some embodiments, T^{A1} can be

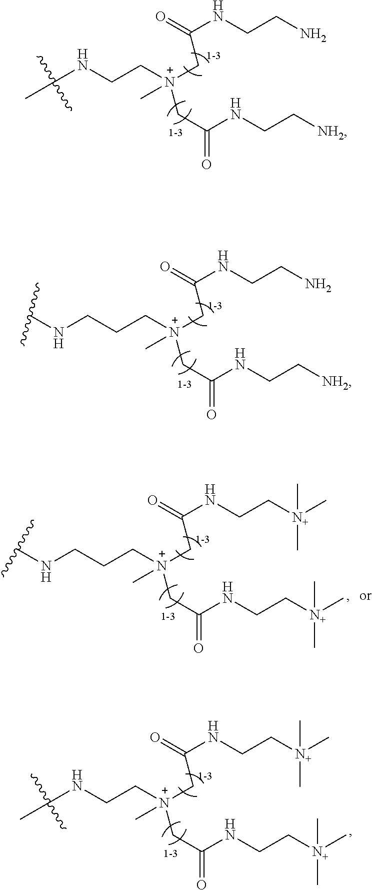

charge balanced with a counterion (e.g., described herein) as necessary as necessary. In some embodiments, T^{A1} can be

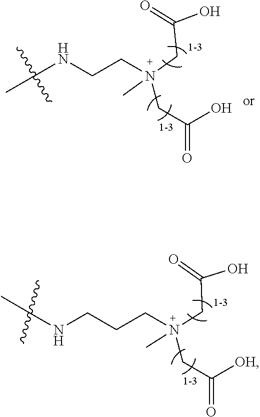

charge balanced with a counterion (e.g., described herein) as necessary. In some embodiments, T^{A1} can be

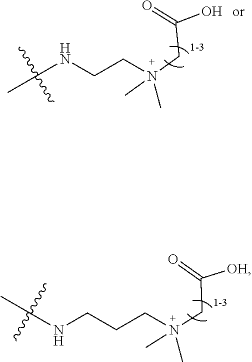

charge balanced with a counterion (e.g., described herein) as necessary. In some embodiments, T^{A1} can be

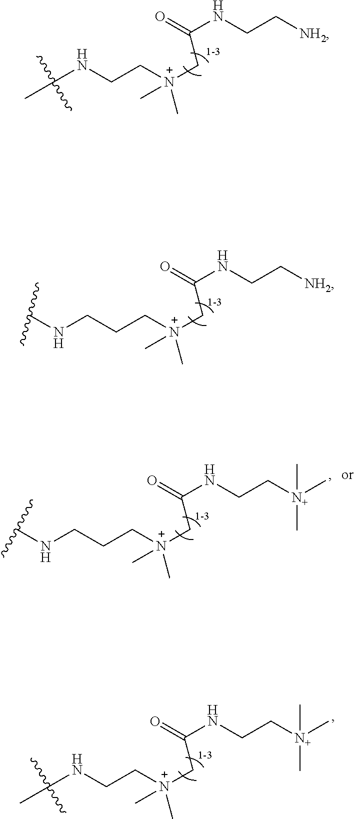

charge balanced with a counterion (e.g., described herein) as necessary. For example, in some embodiments, T^{A1} can be

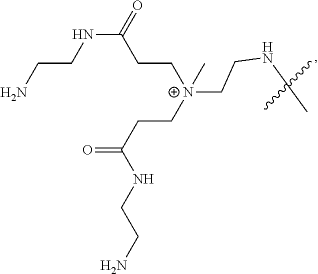 [SMILES: *NCC[N+](C)(CCC(=O)NCCN)CCC(=O)NCCN]

charge balanced with a counterion (e.g., described herein) as necessary. In some embodiments, T^{A1} can be

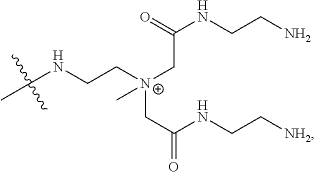 [SMILES: *NCC[N+](C)(CC(=O)NCCN)CC(=O)NCCN]

charge balanced with a counterion (e.g., described herein) as necessary. In some embodiments, T^{A1} can be

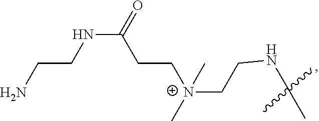 [SMILES: *NCC[N+](C)(C)CCC(=O)NCCN]

charge balanced with a counterion (e.g., described herein) as necessary. In some embodiments, T^{A1} can be

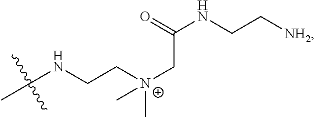 [SMILES: *NCC[N+](C)(C)CC(=O)NCCN]

charge balanced with a counterion (e.g., described herein) as necessary. In some embodiments, T^{A1} can be

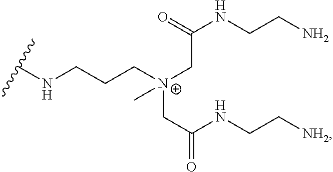 [SMILES: *NCCC[N+](C)(CC(=O)NCCN)CC(=O)NCCN]

charge balanced with a counterion (e.g., described herein) as necessary. In some embodiments, T^{A1} can be

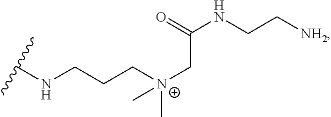 [SMILES: *NCCC[N+](C)(C)CC(=O)NCCN]

charge balanced with a counterion (e.g., described herein) as necessary. In some embodiments, T^{A1} can be

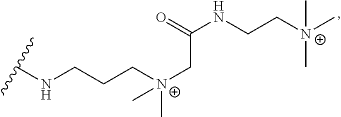 [SMILES: *NCCC[N+](C)(C)CC(=O)NCC[N+](C)(C)C]

charge balanced with a counterion (e.g., described herein) as necessary. In some embodiments, T^{A1} can be

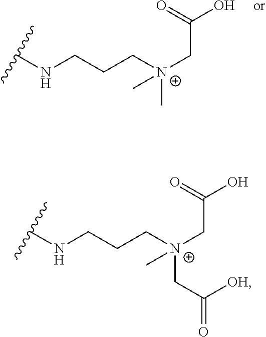 [SMILES: *NCCC[N+](C)(C)CC(=O)O ; *NCCC[N+](C)(CC(=O)O)CC(=O)O]

charge balanced with a counterion (e.g., described herein) as necessary. In some embodiments, T^{A1} can be

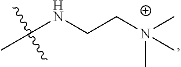

charge balanced with a counterion (e.g., described herein) as necessary. In some embodiments, T^{A1} can be

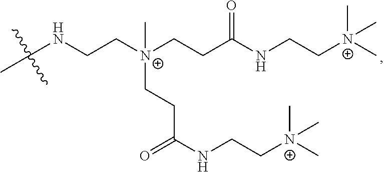 [SMILES: *NCC[N+](C)(CCC(=O)NCC[N+](C)(C)C)CCC(=O)NCC[N+](C)(C)C]

charge balanced with a counterion (e.g., described herein) as necessary. In some embodiments, T^{A1} can be

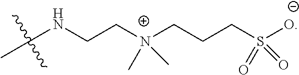

In some preferred embodiments, T^{A} is T^{A1} or T^{A1}-L^{B}-, whrein T^{A1} is

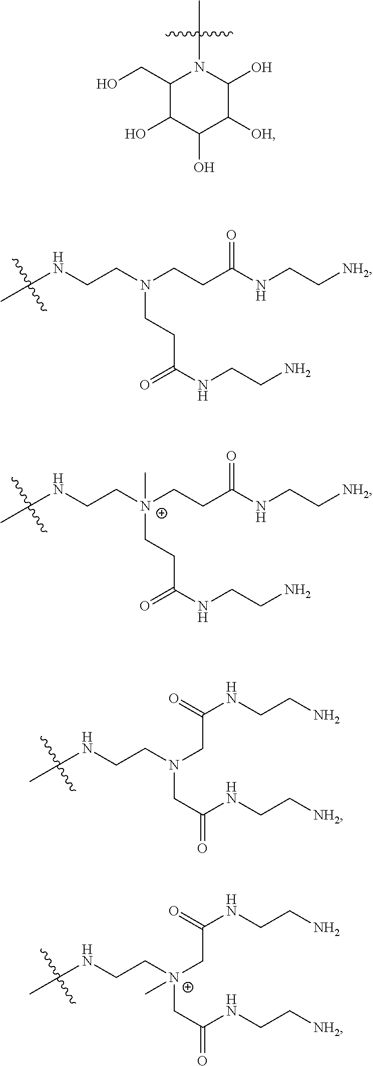 [SMILES: *N1C(O)C(O)C(O)C(O)C1CO ; *NCCN(CCC(=O)NCCN)CCC(=O)NCCN ; *NCC[N+](C)(CCC(=O)NCCN)CCC(=O)NCCN ; *NCCN(CC(=O)NCCN)CC(=O)NCCN ; *NCC[N+](C)(CC(=O)NCCN)CC(=O)NCCN]

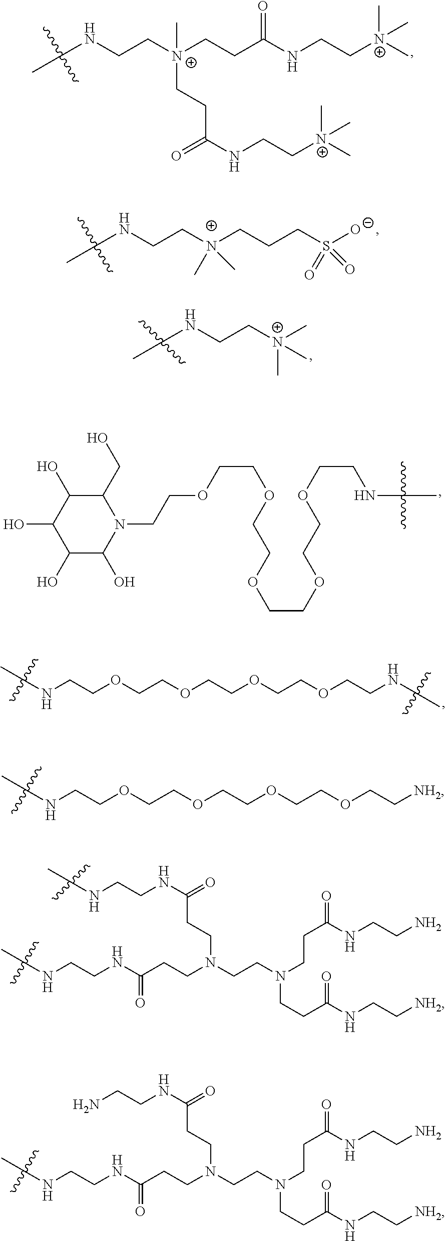

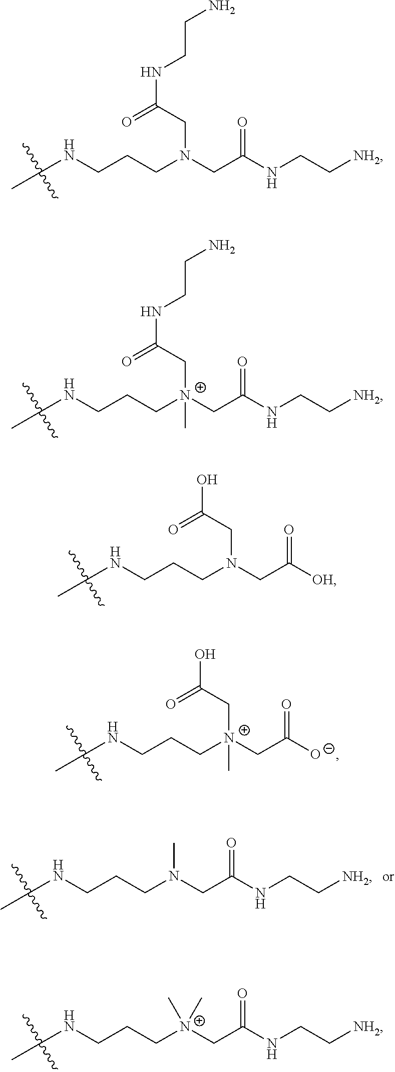 [SMILES: *NCCCN(CC(=O)NCCN)CC(=O)NCCN ; *NCCC[N+](C)(CC(=O)NCCN)CC(=O)NCCN ; *NCCCN(CC(=O)O)CC(=O)O ; *NCCC[N+](C)(CC(=O)[O-])CC(=O)O ; *NCCCN(C)CC(=O)NCCN ; *NCCC[N+](C)(C)CC(=O)NCCN]

and wherein L^{B} is —SO₂—, —C(═O)—, or a moiety selected from:

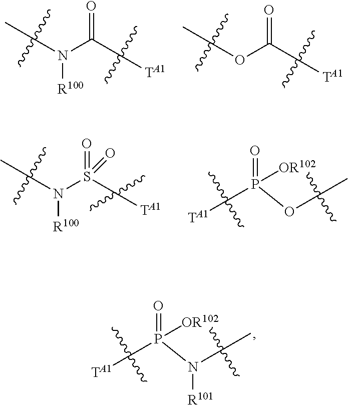

wherein R¹⁰⁰, R¹⁰¹ and R¹⁰² at each occurrence is independently hydrogen, optionally substituted alkyl, or optionally substituted cycloalkyl,
wherein T^{A} is charge balanced with a counterion (e.g., described herein) as necessary.
   Examples of L^{A}-T^{A}

The covalent bond formed between L^{A} and T^{A} is not particularly limited. For example, in some embodiments, the covalent bond(s) between the terminal atom(s) of T^{A} and the end carbon or nitrogen atom(s) of L^{A} is an amide bond. In some embodiments, the covalent bond(s) between the terminal atom(s) of T^{A} and the end atom of L^{A} is a non-amide carbon-nitrogen bond, an ester bond, a non-ester carbon-oxygen bond, a carbon-carbon bond, or a carbon-sulfur bond.

In some preferred embodiments, the compound of Formula II can be characterized as having a Formula II-2, II-3, II-4, II-5, II-6, II-7, II-8, or II-9:

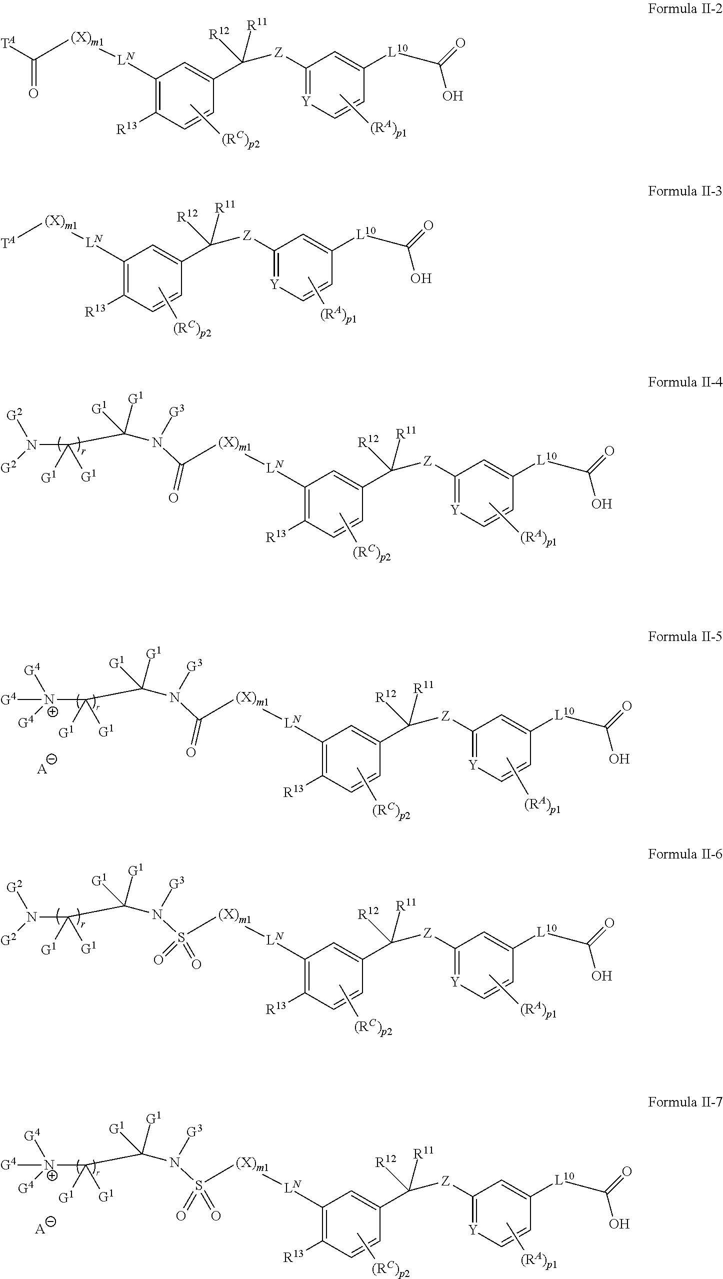

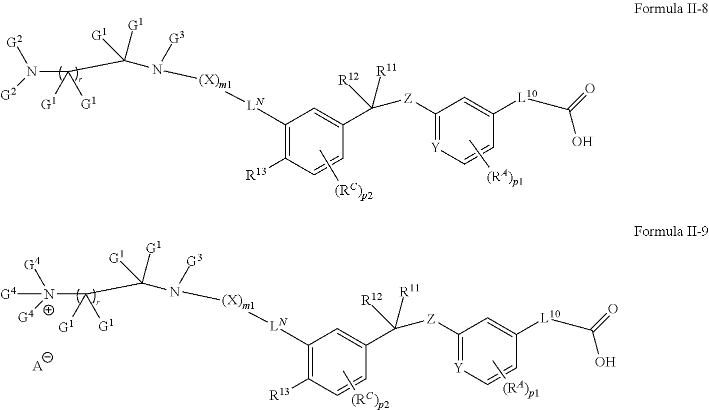

wherein:
X at each occurrence is independently CR₂, C(═O), —C(R)═C(R)—,

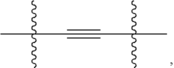 [SMILES: *C#C*]

SiR₂, O, S, SO₂, NR, [NR₂]⁺, or a ring structure, preferably 3-10 membered ring structure, wherein R at each occurrence is independently hydrogen, halogen, optionally substituted C₁₋₄ alkyl, or optionally substituted C₁₋₄ alkoxy,
the integer m1 is at least 12, such as 12-50 (e.g., 12, 14, 16, 18, 20, 22, 24, 30, 40, 50, or any range or value between the recited values),
wherein the hydrophobicity of (X)ₘ₁ is characterized in that (i) when neither of the two end X groups are C(O) or SO₂, then H—(X)ₘ₁—H has a cLogP of at least 3, such as between 3-15, preferably at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc.; (ii) when one end X group is C(O) or SO₂, and the other is not C(O) or SO₂, then the corresponding compound H—(X)ₘ₁—OH has a cLogP of at least 3, such as between 3-15, preferably at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc., the OH being bonded with C(O) or SO₂; or (iii) when both end X groups are C(O) or SO₂, then the corresponding compound HO—(X)ₘ₁—OH has a cLogP of at least 3, such as between 3-15, preferably at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc., the OH being bonded with C(O) or SO₂;
G¹ at each occurrence is independently hydrogen, halogen, optionally substituted C₁₋₄ alkyl, or optionally substituted C₁₋₄ heteroalkyl (such as optionally substituted C₁₋₄ alkoxy), or two gem G¹ group can join to form an ═O, ═NH, or ═N(C₁₋₄ alkyl), or two or more G¹ groups can join to form a ring structure,
the integer r is 1-10, such as 1 or 2,
G² at each occurrence is independently hydrogen, G¹⁰, C(O)-G¹⁰, SO₂G¹⁰, C(O)—NH-G¹⁰, or SO₂NHG¹⁰, C(O)—O-G¹⁰, or SO₂OG¹⁰, wherein G¹⁰ at each occurrence is independently an optionally substituted alkyl, an optionally substituted heteroalkyl, or an optionally substituted 3-10 membered ring structure, which can be carbocyclic, heterocyclic, aromatic, heteroaromatic, or a combination thereof,
G³ is hydrogen or an optionally substituted alkyl (e.g., C₁₋₄ alkyl);
G⁴ at each occurrence is independently hydrogen or G¹⁰, wherein G¹⁰ at each occurrence is independently an optionally substituted alkyl, an optionally substituted heteroalkyl, or an optionally substituted 3-10 membered ring structure, which can be carbocyclic, heterocyclic, aromatic, heteroaromatic, or a combination thereof,
or two G² or one G² and one G¹ or one G¹ and G³ can be joined to form a ring structure such as a 3-10 membered ring structure,
or two G⁴ or one G⁴ and one G¹ or one G¹ and G³ can be joined to form an optionally substituted 3-10 membered ring structure,
A⁻ is a counterion, preferably, a pharmaceutically acceptable anion, as necessary to balance the overall charge of the compound,
wherein the fragment

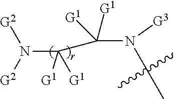

is further characterized in that (i) when the CG¹G¹ group next to the NG³ is not C(═O), the corresponding compound

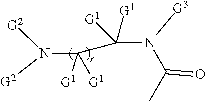

has a cLogP less than 1, preferably, less than 0 (e.g., less than −1, less than −2, less than −3, less than −3.5, less than −4, or even lower), or (ii) when the CG¹G¹ group next to the NG³ is C(═O), the corresponding compound

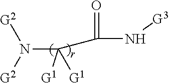

has a cLogP less than 1, preferably, less than 0 (e.g., less than −1, less than −2, less than −3, less than −3.5, less than −4, or even lower),
wherein the fragment

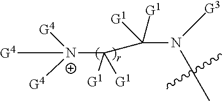

is characterized in that (i) when the CG¹G¹ group next to the NH is not C(═O), the corresponding compound

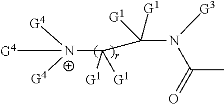

has a cLogP less than 1, preferably, less than 0 (e.g., less than −1, less than −2, less than −3, less than −3.5, less than −4, or even lower), or (ii) when the CG¹G¹ group next to the NH is C(═O), the corresponding compound

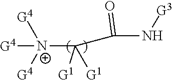

has a cLogP less than 1, preferably, less than 0 (e.g., less than −1, less than −2, less than −3, less than −3.5, less than −4, or even lower), and
wherein the variables T^{A}, L^{N}, L¹⁰, R¹¹, R¹², R¹³, R^{A}, R^{C}, Y, Z, p1, and p2 are defined herein, which include any of those described and preferred herein in any combinations. In some embodiments, L^{N} is null. In some embodiments, L^{N} is null, and two instances of X attached to the phenyl ring can be

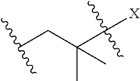

(X is shown to show direction of connection), and the remaining instances of X are as defined herein. In some embodiments, L^{N} is a branched or straight chained C₁₋₆ alkyl alkylene, such as

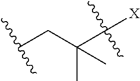

(X is shown to show direction of connection). In some embodiments, G¹ at each occurrence is hydrogen. In some embodiments, one or more instances of G¹ is a C₁₋₄ alkyl such as methyl. In some embodiments, the integer r is 1, 2, 3, 4, or 5. In some embodiments, one or more instances of G² can have a structure of

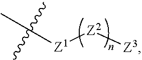

wherein n is an integer of 0-100, preferably, 0-10 (e.g., 0-4, 1-5 or 2-6 etc.), Z¹ is CH₂, C(O), SO, SO₂, CH(C₁₋₄ alkyl), C(C₁₋₄ alkyl)(C₁₋₄ alkyl) or 3-10 membered ring structure, Z² at each occurrence is independently CH₂, O, S, SO, SO₂, C(O), CH(C₁₋₄ alkyl), C(C₁₋₄ alkyl)(C₁₋₄ alkyl), NH, N(C₁₋₄ alkyl), [N(C₁₋₄ alkyl)(C₁₋₄ alkyl)]⁺ or 3-10 membered ring structure, and Z³ is H, C₁₋₄ alkyl, OH, SO₃H, COOH, NH₂, NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, SO₂NH₂, CONH(C₁₋₄ alkyl), CON(C₁₋₄ alkyl)(C₁₋₄ alkyl), SO₂NH(C₁₋₄ alkyl), or SO₂N(C₁₋₄ alkyl)(C₁₋₄ alkyl), wherein each of the C14 alkyl is independently selected and optionally substituted, for example, with 1-3 G²⁰ groups defined herein. In some embodiments, none of the Z² is a 3-10 membered ring structure. In some embodiments, one or more instances of Z² is a 3-10 membered ring structure, such as a nitrogen containing ring,

for example,

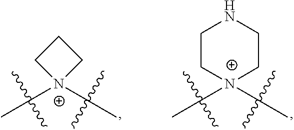 [SMILES: *[N+]1(*)CCC1 ; *[N+]1(*)CCNCC1]

etc. In some embodiments, the Z² directly connects to Z³ is a 3-10 membered ring structure, such as a nitrogen containing ring, for example,

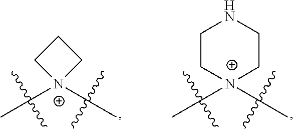 [SMILES: *[N+]1(*)CCC1 ; *[N+]1(*)CCNCC1]

etc. Preferably, at most 2 instances of Z² is a 3-10 membered ring structure. In some embodiments, G² at each occurrence is independently hydrogen, an optionally substituted C₁₋₆ alkyl or an optionally substituted C₁₋₆ heteroalkyl, for example, when substituted, the C₁₋₆ alkyl can be substituted with 1-3 substituents independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), and SO₂N(C₁₋₄ alkyl)(C₁₋₄ alkyl), wherein each of the C₁₋₄ alkyl is independently selected and optionally substituted, for example, with 1-3 G²⁰ groups defined herein. In some embodiments, G⁴ at each occurrence independently can have a structure of

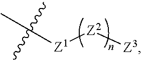

wherein n is an integer of 0-100, preferably, 0-10 (e.g., 0-4, 1-5 or 2-6 etc.), Z¹ is CH₂, C(O), SO₂, CH(C₁₋₄ alkyl), or C(C₁₋₄ alkyl)(C₁₋₄ alkyl), Z² at each occurrence is independently CH₂, O, S, SO, SO₂, C(O), CH(C₁₋₄ alkyl), C(C₁₋₄ alkyl)(C₁₋₄ alkyl), NH, N(C₁₋₄ alkyl), [N(C₁₋₄ alkyl)(C₁₋₄ alkyl)]⁺ or 3-10 membered ring structure, and Z³ is H, C₁₋₄ alkyl, OH, SO₃H, COOH, NH₂, NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, SO₂NH₂, CONH(C₁₋₄ alkyl), CON(C₁₋₄ alkyl)(C₁₋₄ alkyl), SO₂NH(C₁₋₄ alkyl), or SO₂N(C₁₋₄ alkyl)(C₁₋₄ alkyl), wherein each of the C₁₋₄ alkyl is independently selected and optionally substituted, for example, with 1-3 G²⁰ groups defined herein. Preferably, at most 2 instances of Z² is a 3-10 membered ring structure. In some embodiments, none of the Z² is a 3-10 membered ring structure. In some embodiments, one instance of Z² is a 3-10 membered ring structure, such as a nitrogen containing ring, for example,

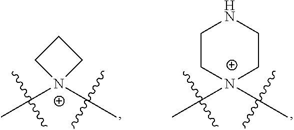 [SMILES: *[N+]1(*)CCC1 ; *[N+]1(*)CCNCC1]

etc. In some embodiments, G⁴ at each occurrence is independently hydrogen, an optionally substituted C₁₋₆ alkyl or an optionally substituted C₁₋₆ heteroalkyl, for example, when substituted, the C₁₋₆ alkyl can be substituted with 1-3 substituents independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), and SO₂N(C₁₋₄ alkyl)(C1-4 alkyl), wherein each of the C₁₋₄ alkyl is independently selected and optionally substituted, for example, with 1-3 G²⁰ groups defined herein. The G²⁰ group herein at each occurrence can be independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), SO₂N(C₁₋₄ alkyl)(C14 alkyl), or 3-10 membered ring structure, wherein each of the C₁₋₄ alkyl is independently selected and optionally substituted, for example, with 1-3 G²¹ groups defined herein. The G²¹ group herein at each occurrence can be independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), SO₂N(C₁₋₄ alkyl)(C₁₋₄ alkyl) or 3-10 membered ring structure. In some embodiments, one or more instances of G⁴ is C₁₋₄ alkyl, such as methyl. In some embodiments, one or more instances of G⁴ is C₁₋₄ alkyl, optionally substituted with COOH or an amide derivative, such as CONH—(C₁₋₄ alkylene)-NH₂ or CONH—(C₁₋₄ alkylene)-[N(C₁₋₄ alkyl)(C₁₋₄ alkyl)(C₁₋₄ alkyl)]⁺. For example, in some embodiments, one or two instances of G⁴ is —(C₁₋₄ alkylene)-COOH, such as —CH₂COOH, and any remaining instance(s) of G⁴ is C₁₋₄ alkyl. In some embodiments, one or two instances of G⁴ is —(C₁₋₄ alkylene)-CONH—(C₁₋₄ alkylene)-NH₂, such as

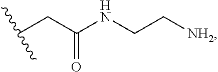 [SMILES: *CC(=O)NCCN]

and any remaining instance(s) of G⁴ is C₁₋₄ alkyl. In some embodiments, one or two instances of G⁴ is —(C₁₋₄ alkylene)-CONH—(C₁₋₄ alkylene)-[N(C₁₋₄ alkyl)(C₁₋₄ alkyl)(C₁₋₄ alkyl)]⁺, such as

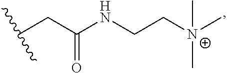 [SMILES: *CC(=O)NCC[N+](C)(C)C]

and any remaining instance(s) of G⁴ is C₁₋₄ alkyl. In preferred embodiments, G³ is hydrogen. In some embodiments, X at each occurrence is independently CH₂, CHCH3, or C(CH₃)₂. In some embodiments, X at each occurrence is CH₂. In some embodiments, up to 6 instances (e.g., 1, 2, 3, 4, 5, or 6) of X is O, and the remaining X is CH₂, CHCH3, or C(CH₃)₂. In some embodiments, up to 10 instances of X is —CH—CH—, and the remaining X is CH₂, CHCH3, or C(CH₃)₂. In some embodiments, the moiety of (X)ₘ₁ has one or more structural features selected from the following: (i) two consecutive X together form-C(O)O— or —C(O) NR—(e.g., C(O)NH or C(O) NCH3); (ii) one or more instances of X can have a ring structure selected from cyclopropane, cyclobutane, bicyclobutane [1.1], cyclopentane, cyclohexane or cycloheptane; and (iii) one or more instances of X can have a ring structure selected from phenyl, or 5- or 6-membered heteroaryl, such as triazole. In some embodiments, within the moiety of (X)ₘ₁, there can be 0, 1, or 2 instances of ring structure, preferably 3-10 membered ring structure. In some embodiments, within the moiety of (X)ₘ₁, there is one triazole ring. For example, in some embodiments, the moiety of (X)ₘ₁ can have a structure according to

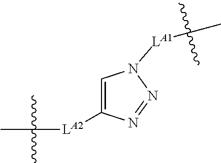

as defined and preferred herein. For example, in some embodiments, L^{A1} and L^{A2} are each independently a bond, —C₁₋₃₀ alkylene-, or —C₁₋₃₀ heteroalkylene-containing 1-6 heteroatoms independently selected from O, N, and S, wherein the sulfur atom(s), if present, is optionally oxidized, wherein the total number of non-hydrogen atoms of (X)ₘ₁ is between 15-50, such as 18, 20, 25, 30, 35, 40, 45, or 50, or any ranges between the recited value. In some embodiments, L^{A1} is a bond. In some embodiments, L^{A1} is a C₁₋₂₀ alkylene, such as a linear alkylene, (CH₂)₁₋₂₀. In some embodiments, L^{A1} is a C₁₋₁₀ heteroalkylene having 1-3 oxygen atoms, such as —O—(C₁₋₁₀ alkylene)-, etc. In some embodiments, L^{A2} is a bond. In some embodiments, L^{A2} is a C₁₋₂₀ alkylene, such as a linear alkylene, (CH₂)₁₋₂₀. In some embodiments, L^{A2} is a C₁₋₁₀ heteroalkylene having 1-3 oxygen atoms, such as —O—(C₁₋₁₀ alkylene)-, etc. Either of L^{A1} and L^{A2} can be attached to L^{N} in Formula II-2 to II-9 above (It should be clear that this does not require L^{N} to actually exist, and when L^{N} is null, it should be understood that the L^{A1} or L^{A2} can be directly attached to the phenyl ring ortho to R¹³ in Formula II-2 to II-9 above).

### Residue of GPR40 Agonist

The variables L¹⁰, R¹¹, R¹², R¹³, R^{A}, R^{C}, Y, Z, HET, p1, and p2 in Formula II or II-B are not particularly limited. However, in preferred embodiments, the variables in Formula II or II-B are such that at least one corresponding compound according to Formula GPR-2,

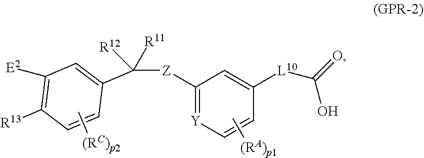

### or Formula GPR-2B,

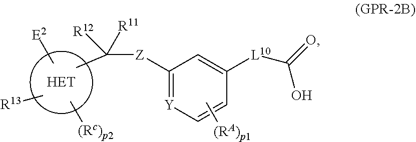

wherein E2 is E2A or L^{N}-E2A, wherein E2A is hydrogen, N3,

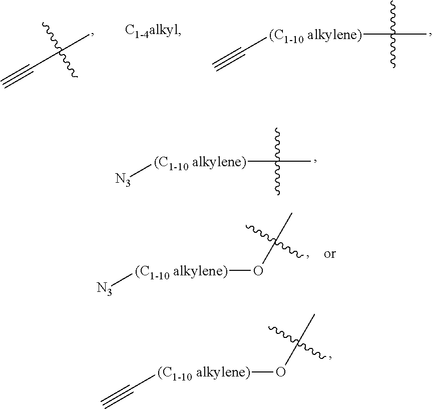

and L^{N} is defined herein (such as null or a C₁₋₆ alkyl alkylene), is a GPR40 agonist, preferably, having an EC50 of less than 100 nM as measured according to Biological Example 1 herein.

Typically, p1 in Formula II or II-B is 0.

In some embodiments, p1 in Formula II or II-B is 1.

Typically, R^{A} at each occurrence is independently F, Cl, CN, C₁₋₄ alkyl optionally substituted with 1-3 fluorine, or C₁₋₄ alkoxy optionally substituted with 1-3 fluorine.

Typically, p2 in Formula II or II-B is 0.

In some embodiments, p2 in Formula II or II-B is 1, and R^{C} is F, Cl, CN, C₁₋₄ alkyl optionally substituted with 1-3 fluorine, or C₁₋₄ alkoxy optionally substituted with 1-3 fluorine.

In Formula II or II-B, Y is typically CH.

Preferably, Z in Formula II or II-B is O.

In some embodiments, R¹¹ and R¹² are both hydrogen.

In some preferred embodiments, L¹⁰ is characterized as having a structure of

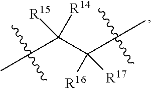

wherein CR₁₆R¹⁷ is bonded to the COOH group, and wherein:
R¹⁴ is hydrogen, C₁₋₆ alkyl, C₂₋₆ alkenyl, C₂₋₆ alkynyl, C₃₋₆ cycloalkyl, phenyl, 5 or 6 membered heteroaryl, or 3-7 membered heterocyclyl, wherein each of the alkyl, alkenyl, alkynyl, cycloalkyl, phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy, and
R¹⁵, R¹⁶ and R¹⁷ are each independently hydrogen or C₁₋₄ alkyl; or
R¹⁴ and R¹⁵ are joined to form a 3-7 membered ring with 0, 1, or 2 heteroatoms selected from O, N, or S. In some embodiments, R¹⁶ and R¹⁷ are both hydrogen, or one of R¹⁶ and R¹⁷ is hydrogen and the other of R¹⁶ and R¹⁷ is methyl. In some embodiments, one of R¹⁴ and R¹⁵ is hydrogen, and the other of R¹⁴ and R¹⁵ is C₁₋₆ alkyl, C₂₋₆ alkenyl, C₂₋₆ alkynyl, or C₃₋₆ cycloalkyl. In some embodiments, R¹⁴ and R¹⁵ are joined to form a C₃₋₆ cycloalkyl.

For example, in some embodiments, L¹⁰ is

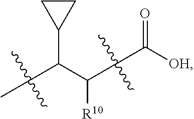

wherein R¹⁰ is hydrogen or C₁₋₄ alkyl, such as methyl.
R¹³ in Formula II or II-B is typically an optionally substituted phenyl or an optionally substituted 5 or 6 membered heteroaryl having 1-4 ring heteroatoms. In some embodiments, R¹³ in Formula II or II-B can also be hydrogen.

In some embodiments, R¹³ is an optionally substituted phenyl. In some embodiments, R¹³ is a phenyl ring, which is unsubstituted. In some embodiments, R¹³ is a phenyl ring, which is substituted with 1-3 substituents independently selected from F, Cl, CN, OH, C₁₋₆ alkyl, C₁₋₆ alkyl heteroalkyl, C₃₋₆ cycloalkyl, C₁₋₆ alkyl alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), and C1-4 alkyl optionally substituted with F.

In some embodiments, R¹³ is an optionally substituted 6-membered heteroaryl ring. In some embodiments, R¹³ is a 6-membered heteroaryl ring, such as a pyridyl ring,

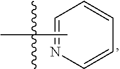

which is optionally substituted with 1-3 substituents independently selected from F, CI, CN, OH, C₁₋₆ alkyl, C₁₋₆ alkyl heteroalkyl, C₃₋₆ cycloalkyl, C₁₋₆ alkyl alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), and C₁₋₄ alkyl optionally substituted with F.

In some preferred embodiments, the compound of Formula II or II-B can be characterized as having a Formula II-1-A or II-B-2, respectively:

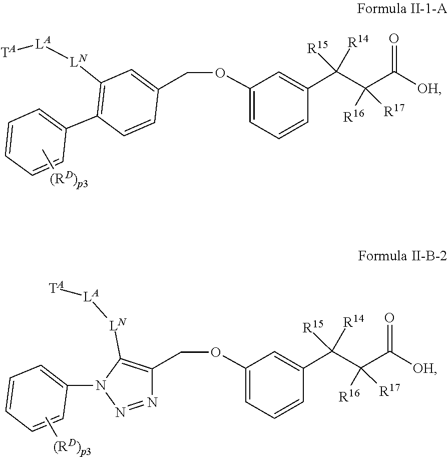

wherein:
R¹⁴ is hydrogen, C₁₋₆ alkyl, C₂₋₆ alkenyl, C₂₋₆ alkynyl, C₃₋₆ cycloalkyl, phenyl, 5 or 6 membered heteroaryl, or 3-7 membered heterocyclyl, wherein each of the alkyl, alkenyl, alkynyl, cycloalkyl, phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy, and R¹⁵, R¹⁶ and R¹⁷ are each independently hydrogen or C₁₋₄ alkyl; or
R¹⁴ and R¹⁵ are joined to form a 3-7 membered ring with 0, 1, or 2 heteroatoms selected from O, N, or S;
R^{D} at each occurrence is independently F, Cl, C₁₋₄ alkyl optionally substituted with 1-3 F, or C₁₋₄ alkoxy optionally substituted with 1-3 F, and
wherein p3 is 0, 1, 2, or 3,
and the variables T^{A}, L^{N}, and L^{A} include any of those described and preferred herein in any combinations. For example, in some embodiments, L^{N} is null. In some embodiments, L^{N} is a branched or straight chained C₁₋₆ alkyl alkylene, such as

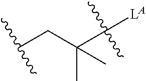

(L^{A} is shown to show direction of connection). In some embodiments, R¹⁶ and R¹⁷ are both hydrogen. In some embodiments, one of R¹⁶ and R¹⁷ is hydrogen and the other of R¹⁶ and R¹⁷ is methyl. In some embodiments, one of R¹⁴ and R¹⁵ is hydrogen, and the other of R¹⁴ and R¹⁵ is C₁₋₆ alkyl, C₂₋₆ alkenyl, C₂₋₆ alkynyl, or C₃₋₆ cycloalkyl. In some embodiments, R¹⁴ and R¹⁵ are joined to form a C₃₋₆ cycloalkyl.

In some preferred embodiments, the compound of Formula II or II-B can be characterized as having a Formula II-1-A-1 or II-B-3, respectively:

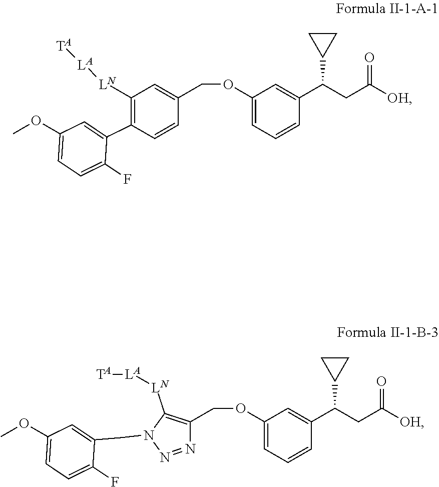

wherein the variables T^{A}, L^{N}, and L^{A} include any of those described and preferred herein in any combinations. For example, in some embodiments, L^{N} is null. In some embodiments, L^{N} is a branched or straight chained C₁₋₆ alkyl alkylene, such as

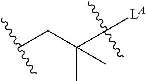

(L^{A} is shown to show direction of connection).

In preferred embodiments, L^{A} in Formula II-1-A (e.g., II-1-A-1), II-B-2, or II-B-3 can be -X₁₂₋₃₀—C(O)—, wherein the C(O) is directly bonded with T^{A}, and wherein X at each occurrence is independently CR₂, —C(R)═C(R)—,

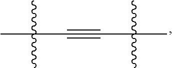 [SMILES: *C#C*]

SiR₂, C(O), O, NR, S, SO₂, or a ring structure, preferably 3-10 membered ring structure, wherein R at each occurrence is independently hydrogen, halogen, optionally substituted C₁₋₄ alkyl, or optionally substituted C₁₋₄ alkoxy, typically hydrogen or C₁₋₄ alkyl, wherein the longest chain length of L^{A} is at least that of —(CH₂)₁₂—, such as at least that of —(CH₂)₁₄—, at least that of —(CH₂)₁₆—, at least that of —(CH₂)₁₈—, and wherein the hydrophobicity of L^{A} can be characterized in that the corresponding compound H-X12-30—C(O)—OH has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc. Preferably, the end X group (i.e., the X group in L^{A} that is furthest away from the C(O) end) is not C(═O) or SO₂. Preferably, the total number of non-hydrogen atoms of L^{A} is between 15-50, such as 18, 20, 25, 30, 35, 40, 45, or 50, or any ranges between the recited value. In some preferred embodiments, none of the X groups is a ring structure. In some preferred embodiments, one instance of X is a ring structure, preferably 3-10 membered ring structure. For example, in some embodiments, one instance of X is triazole and the remaining X groups are each independently CR₂, —C(R)═C(R)—,

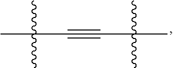 [SMILES: *C#C*]

or O, wherein R at each occurrence is independently hydrogen or C₁₋₄ alkyl. In some embodiments, none of the X groups is a ring structure, and each X is independently CR₂, —C(R)═C(R)—,

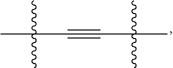 [SMILES: *C#C*]

or O, wherein R at each occurrence is independently hydrogen or C₁₋₄ alkyl (preferably, methyl). In some embodiments, all instances of X is CR₂, wherein R at each occurrence is independently hydrogen or C₁₋₄ alkyl (preferably, methyl). In some embodiments, up to 6 instances (e.g., 1, 2, 3, 4, 5, or 6) of X can be O. In some embodiments, one or two instances of X is O, and all other instances of X is CR₂, wherein R at each occurrence is independently hydrogen or C₁₋₄ alkyl (preferably, methyl). In some embodiments, one or two instances of X is O, one X is triazole, and all other instances of X is CR₂, wherein R at each occurrence is independently hydrogen or C₁₋₄ alkyl (preferably, methyl). In some embodiments, R is hydrogen.

In some preferred embodiments, T^{A} in Formula II-1-A (e.g., II-1-A-1), II-B-2, or II-B-3 can be

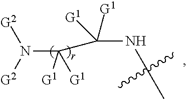

wherein G¹ at each occurrence is independently hydrogen, halogen, optionally substituted C₁₋₄ alkyl, or optionally substituted C₁₋₄ heteroalkyl (such as optionally substituted C₁₋₄ alkoxy), or two gem G¹ group can join to form an ═O, ═NH, or ═N(C₁₋₄ alkyl), or two or more G¹ groups can join to form a ring structure,
the integer r is 1-10, such as 1 or 2,
G² at each occurrence is independently hydrogen, G¹⁰, C(O)-G¹⁰, SO₂G¹⁰, C(O)—NH-G¹⁰, or SO₂NHG¹⁰, C(O)—O-G¹⁰, or SO₂OG¹⁰, wherein G¹⁰ at each occurrence is independently an optionally substituted C₁₋₆ alkyl, an optionally substituted C₁₋₆ heteroalkyl, or an optionally substituted 3-10 membered ring structure, which can be carbocyclic, heterocyclic, aromatic, heteroaromatic, or a combination thereof, wherein the fragment is further characterized in that (i) when the CG¹G¹ group next to the NH is not C(═O), the corresponding compound

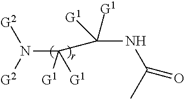

has a cLogP less than 1, preferably, less than 0 (e.g., less than −1, less than −2, less than −3, less than −3.5, less than −4, or even lower), or (ii) when the CG¹G¹ group next to the NH is C(═O), the corresponding compound

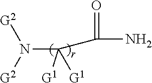

has a cLogP less than 1, preferably, less than 0 (e.g., less than −1, less than −2, less than −3, less than −3.5, less than −4, or even lower).

In some preferred embodiments, T^{A} in Formula II-1-A (e.g., II-1-A-1), II-B-2, or II-B-3 can be characterized as having a structure of:

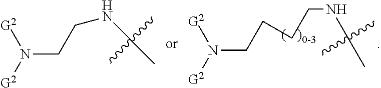

In some embodiments, one or both instances of G² can have a structure of

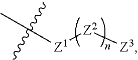

wherein n is an integer of 0-100, preferably, 0-10 (e.g., 0-4, 1-5 or 2-6 etc.), Z¹ is CH₂, C(O), SO, SO₂, CH(C₁₋₄ alkyl), C(C₁₋₄ alkyl)(C₁₋₄ alkyl) or 3-10 membered ring structure, Z² at each occurrence is independently CH₂, O, S, SO, SO₂, C(O), CH(C₁₋₄ alkyl), C(C₁₋₄ alkyl)(C₁₋₄ alkyl), NH, N(C₁₋₄ alkyl), [N(C₁₋₄ alkyl)(C₁₋₄ alkyl)]⁺ or 3-10 membered ring structure, and Z³ is H, C₁₋₄ alkyl, OH, SO₃H, COOH, NH₂, NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, SO₂NH₂, CONH(C₁₋₄ alkyl), CON(C₁₋₄ alkyl)(C₁₋₄ alkyl), SO₂NH(C₁₋₄ alkyl), or SO₂N(C₁₋₄ alkyl)(C₁₋₄ alkyl), wherein each of the C₁₋₄ alkyl is independently selected and optionally substituted, for example, with 1-3 G²⁰ groups defined herein. In some embodiments, none of the Z² is a 3-10 membered ring structure. In some embodiments, one or more instances of Z² is a 3-10 membered ring structure, such as a nitrogen containing ring, for example,

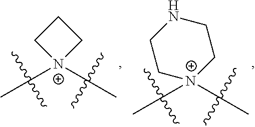 [SMILES: *[N+]1(*)CCC1 ; *[N+]1(*)CCNCC1]

etc. In some embodiments, the Z² directly connects to Z³ is a 3-10 membered ring structure, such as a nitrogen containing ring, for example,

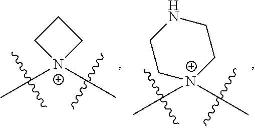 [SMILES: *[N+]1(*)CCC1 ; *[N+]1(*)CCNCC1]

etc. Preferably, at most 2 instances of Z² is a 3-10 membered ring structure. In some embodiments, G² at each occurrence is independently hydrogen, an optionally substituted C₁₋₆ alkyl or an optionally substituted C₁₋₆ heteroalkyl, for example, when substituted, the C₁₋₆ alkyl can be substituted with 1-3 substituents independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), and SO₂N(C₁₋₄ alkyl)(C₁₋₄ alkyl), wherein each of the C₁₋₄ alkyl is independently selected and optionally substituted, for example, with 1-3 G²⁰ groups defined herein. The G²⁰ group herein at each occurrence can be independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), SO₂N(C₁₋₄ alkyl)(C₁₋₄ alkyl), or 3-10 membered ring structure, wherein each of the C₁₋₄ alkyl is independently selected and optionally substituted, for example, with 1-3 G²¹ groups defined herein. The G²¹ group herein at each occurrence can be independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), SO₂N(C₁₋₄ alkyl)(C₁₋₄ alkyl) or 3-10 membered ring structure. In some embodiments, one or more instances of G² is C₁₋₄ alkyl, optionally substituted with COOH or an amide derivative, such as CONH—(C₁₋₄ alkylene)-NH₂ or CONH—(C₁₋₄ alkylene)-[N(C₁₋₄ alkyl)(C₁₋₄ alkyl)(C₁₋₄ alkyl)]⁺. For example, in some embodiments, one or two instances of G² is —(C₁₋₄ alkylene)-COOH, such as —CH₂COOH, and any remaining instance(s) of G² is C₁₋₄ alkyl. In some embodiments, one or two instances of G² is —(C₁₋₄ alkylene)-CONH—(C₁₋₄ alkylene)-NH₂, such

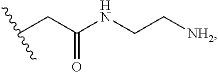 [SMILES: *CC(=O)NCCN]

and any remaining instance(s) of G² is C₁₋₄ alkyl. In some embodiments, one or two instances of G² is —(C₁₋₄ alkylene)-CONH—(C₁₋₄ alkylene)-[N(C₁₋₄ alkyl)(C₁₋₄ alkyl)(C₁₋₄ alkyl)]⁺, such as

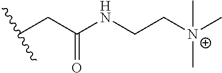 [SMILES: *CC(=O)NCC[N+](C)(C)C]

and any remaining instance(s) of G² is C₁₋₄ alkyl. In some embodiments, T^{A} can be

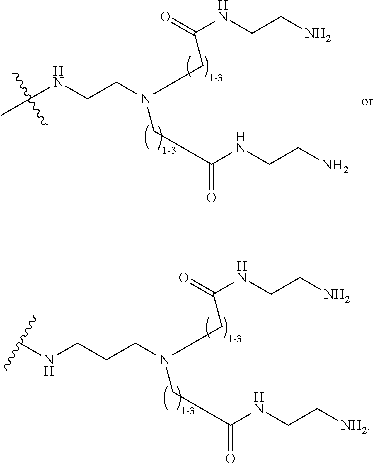

In some embodiments, T^{A} can be

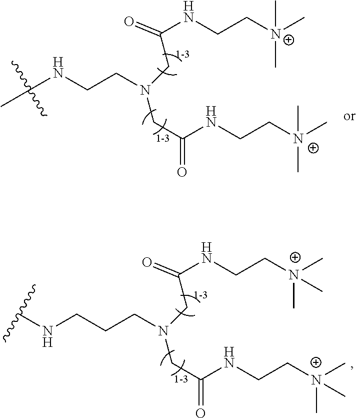

charge balanced with a counterion (e.g., described herein) as necessary. In some embodiments, T^{A} can be

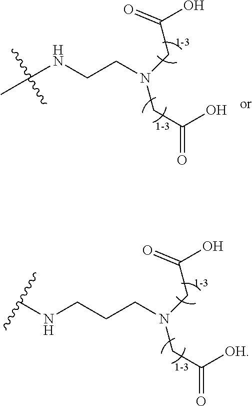

In some embodiments, T^{A} can be

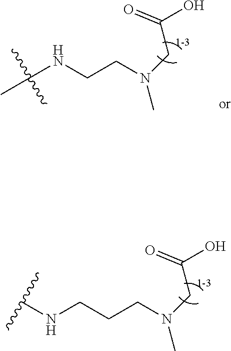

In some embodiments, T^{A} can be

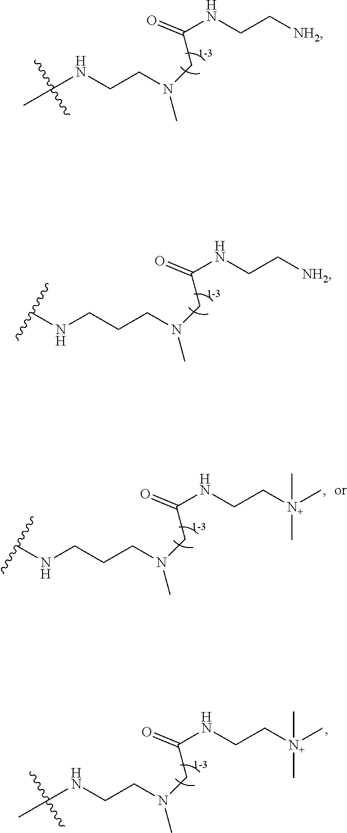

charge balanced with a counterion (e.g., described herein) as necessary. In some embodiments, T^{A} can be

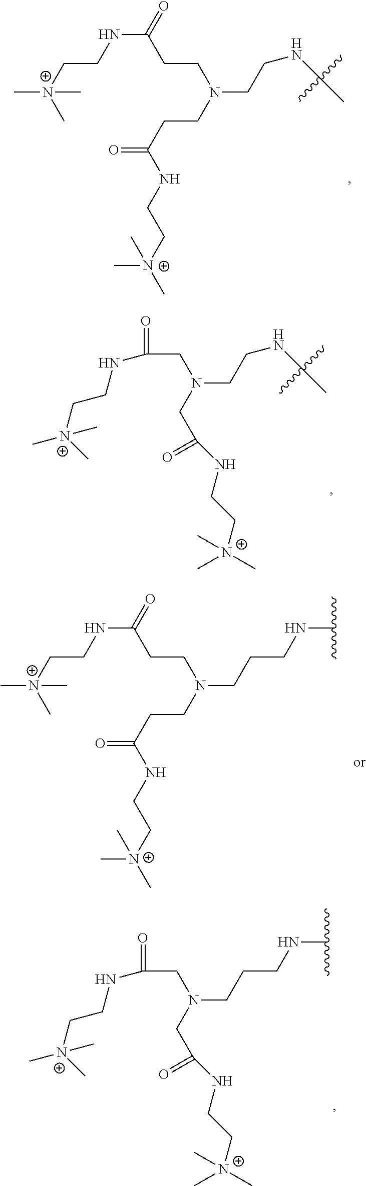 [SMILES: *NCCN(CCC(=O)NCC[N+](C)(C)C)CCC(=O)NCC[N+](C)(C)C ; *NCCN(CC(=O)NCC[N+](C)(C)C)CC(=O)NCC[N+](C)(C)C ; *NCCCN(CCC(=O)NCC[N+](C)(C)C)CCC(=O)NCC[N+](C)(C)C ; *NCCCN(CC(=O)NCC[N+](C)(C)C)CC(=O)NCC[N+](C)(C)C]

charge balanced with a counterion (e.g., described herein) as necessary. For example, in some embodiments, T^{A} can be

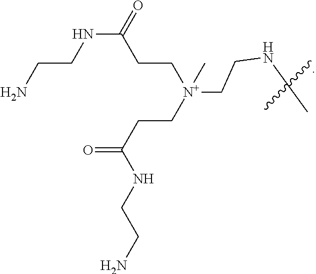 [SMILES: *NCC[N+](C)(CCC(=O)NCCN)CCC(=O)NCCN]

In some embodiments, T^{A} can be

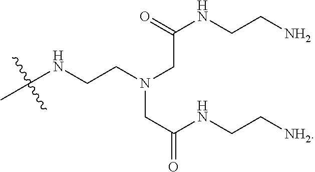 [SMILES: *NCCN(CC(=O)NCCN)CC(=O)NCCN]

In some embodiments, T^{A} can be

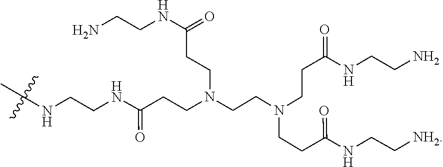 [SMILES: *NCCNC(=O)CCN(CCC(=O)NCCN)CCN(CCC(=O)NCCN)CCC(=O)NCCN]

In some embodiments, T^{A} can be

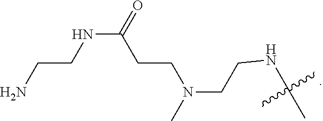 [SMILES: *NCCN(C)CCC(=O)NCCN]

In some embodiments, T^{A} can be

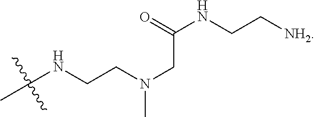 [SMILES: *NCCN(C)CC(=O)NCCN]

In some embodiments, T^{A} can be

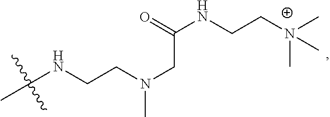 [SMILES: *NCCN(C)CC(=O)NCC[N+](C)(C)C]

charge balanced with a counterion (e.g., described herein) as necessary. In some embodiments, T^{A} can be

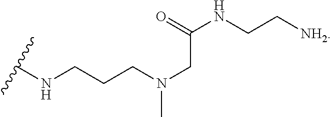 [SMILES: *NCCCN(C)CC(=O)NCCN]

In some embodiments, T^{A} can be

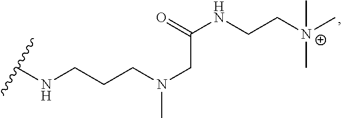 [SMILES: *NCCCN(C)CC(=O)NCC[N+](C)(C)C]

charge balanced with a counterion (e.g., described herein) as necessary. In some embodiments, T^{A} can be

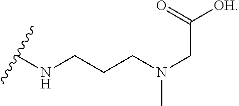 [SMILES: *NCCCN(C)CC(=O)O]

In some preferred embodiments, T^{A} in Formula II-1-A (e.g., II-1-A-1), II-B-2, or II-B-3 can be characterized as having a structure of:

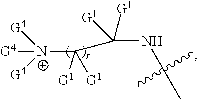

wherein G¹ at each occurrence is independently hydrogen, halogen, optionally substituted C₁₋₄ alkyl, or optionally substituted C₁₋₄ heteroalkyl (such as optionally substituted C₁₋₄ alkoxy), or two gem G¹ group can join to form an —O, ═NH, or ═N(C₁₋₄ alkyl), or two or more G¹ groups can join to form a ring structure,
the integer r is 1-10, such as 1 or 2,
G⁴ at each occurrence is independently hydrogen or G¹⁰, wherein G¹⁰ at each occurrence is independently an optionally substituted C₁₋₆ alkyl, an optionally substituted C₁₋₆ heteroalkyl, or an optionally substituted 3-10 membered ring structure, which can be carbocyclic, heterocyclic, aromatic, heteroaromatic, or a combination thereof, wherein the fragment

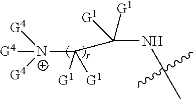

is characterized in that (i) when the CG¹G¹ group next to the NH is not C(═O), the corresponding compound

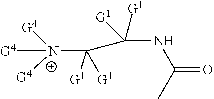

has a cLogP less than 1, preferably, less than 0 (e.g., less than −1, less than −2, less than −3, less than −3.5, less than −4, or even lower), or (ii) when the CG¹G¹ group next to the NH is C(═O), the corresponding compound

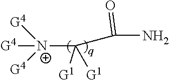

has a cLogP less than 1, preferably, less than 0 (e.g., less than −1, less than −2, less than −3, less than −3.5, less than −4, or even lower).

In some preferred embodiments, T^{A} in Formula II-1-A (e.g., II-1-A-1), II-B-2, or II-B-3 can be characterized as having a structure of:

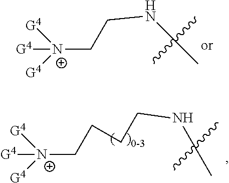

In some embodiments, one or more instances of G⁴ can have a structure of

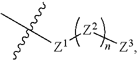

n is an integer of 0-100, preferably, 0-10 (e.g., 0-4, 1-5 or 2-6 etc.), Z¹ is CH₂, C(O), SO₂, CH(C₁₋₄ alkyl), or C(C₁₋₄ alkyl)(C₁₋₄ alkyl), Z² at each occurrence is independently CH₂, O, S, SO, SO₂, C(O), CH(C₁₋₄ alkyl), C(C₁₋₄ alkyl)(C₁₋₄ alkyl), NH, N(C₁₋₄ alkyl), [N(C₁₋₄ alkyl)(C₁₋₄ alkyl)]⁺ or 3-10 membered ring structure, and Z³ is H, C₁₋₄ alkyl, OH, SO₃H, COOH, NH₂, NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, SO₂NH₂, CONH(C₁₋₄ alkyl), CON(C₁₋₄ alkyl)(C₁₋₄ alkyl), SO₂NH(C₁₋₄ alkyl), or SO₂N(C₁₋₄ alkyl)(C₁₋₄ alkyl), wherein each of the C₁₋₄ alkyl is independently selected and optionally substituted, for example, with 1-3 G²⁰ groups defined herein. Preferably, at most 2 instances of Z² is a 3-10 membered ring structure. In some embodiments, none of the Z² is a 3-10 membered ring structure. In some embodiments, one instance of Z² is a 3-10 membered ring structure, such as a nitrogen containing ring, for example,

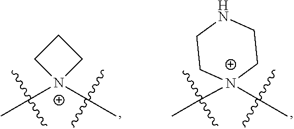 [SMILES: *[N+]1(*)CCC1 ; *[N+]1(*)CCNCC1]

etc. In some embodiments, G⁴ at each occurrence is independently hydrogen, an optionally substituted C₁₋₆ alkyl or an optionally substituted C₁₋₆ heteroalkyl, for example, when substituted, the C₁₋₆ alkyl can be substituted with 1-3 substituents independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), and SO₂N(C₁₋₄ alkyl)(C1. 4 alkyl), wherein each of the C₁₋₄ alkyl is independently selected and optionally substituted, for example, with 1-3 G²⁰ groups defined herein. The G²⁰ group herein at each occurrence can be independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), SO₂N(C₁₋₄ alkyl)(C₁₋₄ alkyl), or 3-10 membered ring structure, wherein each of the C₁₋₄ alkyl is independently selected and optionally substituted, for example, with 1-3 G²¹ groups defined herein. The G²¹ group herein at each occurrence can be independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), SO₂N(C₁₋₄ alkyl)(C₁₋₄ alkyl) or 3-10 membered ring structure. In some embodiments, one or more instances of G⁴ is C₁₋₄ alkyl, such as methyl. In some embodiments, one or more instances of G⁴ is C₁₋₄ alkyl, optionally substituted with COOH or an amide derivative, such as CONH—(C₁₋₄ alkylene)-NH₂ or CONH—(C₁₋₄ alkylene)-[N(C₁₋₄ alkyl)(C₁₋₄ alkyl)(C₁₋₄ alkyl)]⁺. For example, in some embodiments, one or two instances of G⁴ is —(C₁₋₄ alkylene)-COOH, such as —CH₂COOH, and any remaining instance(s) of G⁴ is C₁₋₄ alkyl. In some embodiments, one or two instances of G⁴ is —(C₁₋₄ alkylene)-CONH—(C₁₋₄ alkylene)-NH₂, such as

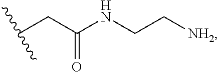 [SMILES: *CC(=O)NCCN]

and any remaining instance(s) of G⁴ is C₁₋₄ alkyl. In some embodiments, one or two instances of G⁴ is —(C₁₋₄ alkylene)-CONH—(C₁₋₄ alkylene)-[N(C₁₋₄ alkyl)(C₁₋₄ alkyl)(C₁₋₄ alkyl)]⁺, such as

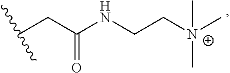 [SMILES: *CC(=O)NCC[N+](C)(C)C]

and any remaining instance(s) of G⁴ is C₁₋₄ alkyl. In some embodiments, T^{A} can be

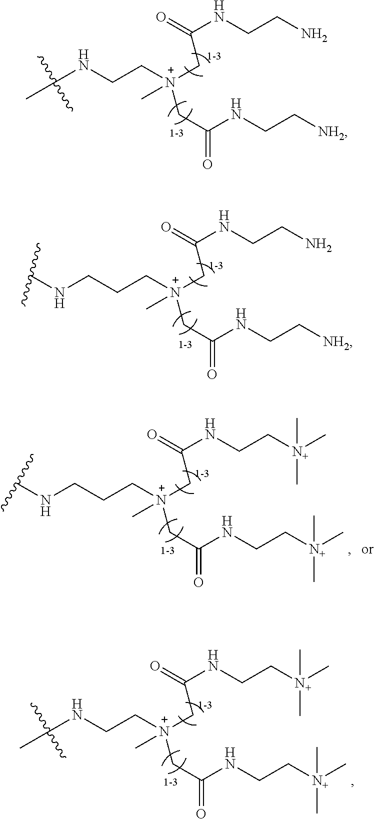

charge balanced with a counterion (e.g., described herein) as necessary. In some embodiments, T^{A} can be

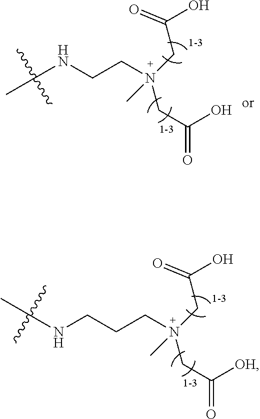

charge balanced with a counterion (e.g., described herein) as necessary. In some embodiments, T^{A} can be

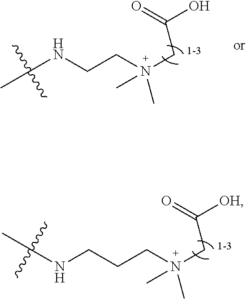

charge balanced with a counterion (e.g., described herein) as necessary. In some embodiments, T^{A} can be

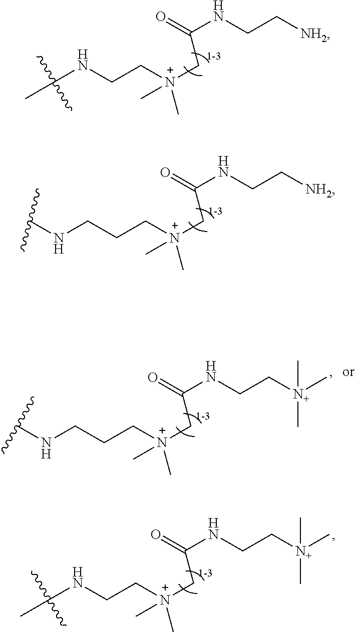

charge balanced with a counterion (e.g., described herein) as necessary. For example, in some embodiments, T^{A} can be

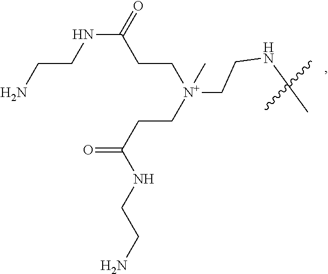 [SMILES: *NCC[N+](C)(CCC(=O)NCCN)CCC(=O)NCCN]

charge balanced with a counterion (e.g., described herein) as necessary. In some embodiments, T^{A} can be

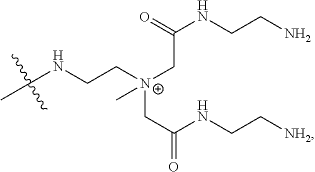 [SMILES: *NCC[N+](C)(CC(=O)NCCN)CC(=O)NCCN]

charge balanced with a counterion (e.g., described herein) as necessary. In some embodiments, T^{A} can be

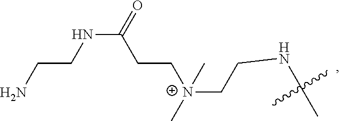 [SMILES: *NCC[N+](C)(C)CCC(=O)NCCN]

charge balanced with a counterion (e.g., described herein) as necessary. In some embodiments, T^{A} can be

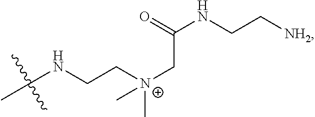 [SMILES: *NCC[N+](C)(C)CC(=O)NCCN]

charge balanced with a counterion (e.g., described herein) as necessary. In some embodiments, T^{A} can be

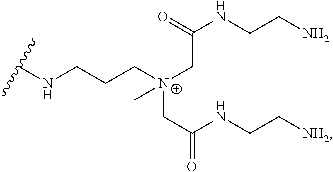 [SMILES: *NCCC[N+](C)(CC(=O)NCCN)CC(=O)NCCN]

charge balanced with a counterion (e.g., described herein) as necessary. In some embodiments, T^{A} can be

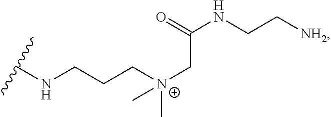 [SMILES: *NCCC[N+](C)(C)CC(=O)NCCN]

charge balanced with a counterion (e.g., described herein) as necessary. In some embodiments, T^{A} can be

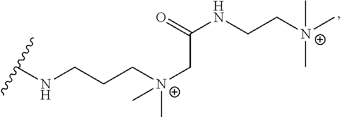 [SMILES: *NCCC[N+](C)(C)CC(=O)NCC[N+](C)(C)C]

charge balanced with a counterion (e.g., described herein) as necessary. In some embodiments, T^{A} can be

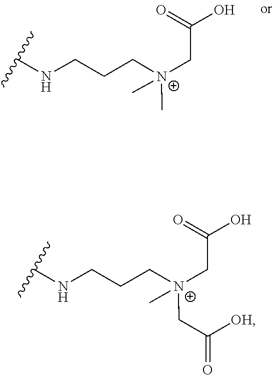 [SMILES: *NCCC[N+](C)(C)CC(=O)O ; *NCCC[N+](C)(CC(=O)O)CC(=O)O]

charge balanced with a counterion (e.g., described herein) as necessary. In some embodiments, T^{A} can be

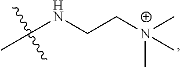 [SMILES: *NCC[N+](C)(C)C]

charge balanced with a counterion (e.g., described

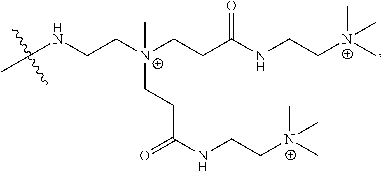 [SMILES: *NCC[N+](C)(CCC(=O)NCC[N+](C)(C)C)CCC(=O)NCC[N+](C)(C)C]

herein) as necessary. In some embodiments, T^{A} can be charge balanced with a counterion (e.g., described herein) as necessary. In some embodiments, T^{A} can be

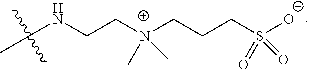 [SMILES: *NCC[N+](C)(C)CCCS(=O)(=O)[O-]]

In some preferred embodiments, the present disclosure also provides a compound of Formula II-4-A or II-5-A, or a pharmaceutically acceptable salt or ester thereof:

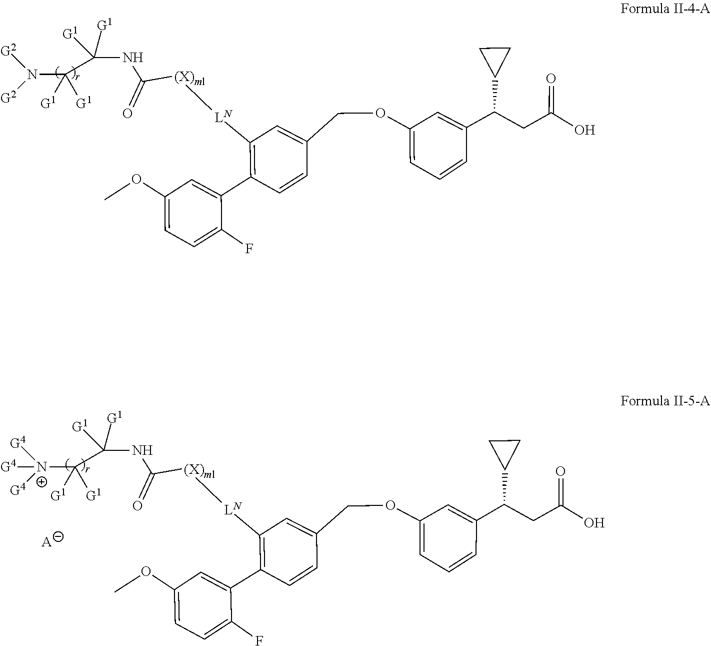

wherein X, m1, L^{N}, G¹, G², G⁴, r, and A⁻ are defined herein, which include any of those described and preferred herein in connection with Formula II (e.g., II-1, II-4, II-5, II-1-A, II-1-A-1, etc.), in any combinations. For example, in some embodiments, L^{N} is a branched or straight chained C₁₋₆ alkyl, such as

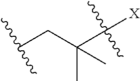

(X is shown to show direction of connection). In some embodiments, L^{N} is null. In some embodiments, X at each occurrence can be independently CR₂, —C(R)═C(R)—,

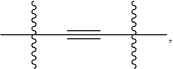 [SMILES: *C#C*]

SiR₂, C(O), O, NR, S, SO₂, or a ring structure, preferably 3-10 membered ring structure, wherein R at each occurrence is independently hydrogen, halogen, optionally substituted C₁₋₄ alkyl, or optionally substituted C₁₋₄ alkoxy, typically hydrogen or C₁₋₄ alkyl, preferably, the end X groups of the (X)ₘ₁ moiety are not C(═O) or SO₂. In some embodiments, the integer m1 can be 12-50, such as 14-50, 16-30, etc. Preferably, the total number of non-hydrogen atoms of the (X)ₘ₁ moiety is between 15-50, such as 18, 20, 25, 30, 35, 40, 45, or 50, or any ranges between the recited value. In some preferred embodiments, none of the X groups is a ring structure. In some preferred embodiments, one instance of X is a ring structure, preferably 3-10 membered ring structure. For example, in some embodiments, one instance of X is triazole and the remaining X groups are each independently CR₂, —C(R)—C(R)—,

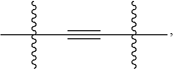 [SMILES: *C#C*]

or O, more preferably, CR₂ or O, wherein R at each occurrence is independently hydrogen or C₁₋₄ alkyl. In some embodiments, none of the X groups is a ring structure, and each X is independently CR₂, —C(R)═C(R)—,

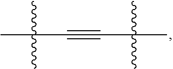 [SMILES: *C#C*]

or O, more preferably, CR₂ or O, wherein R at each occurrence is independently hydrogen or C₁₋₄ alkyl. In some embodiments, up to 6 instances (e.g., 1, 2, 3, 4, 5, or 6) of X can be O. In some embodiments, all instances of X is CR₂, wherein R at each occurrence is independently hydrogen or C₁₋₄ alkyl (preferably, methyl). In some embodiments, one or two instances of X is O, and all other instances of X is CR₂, wherein R at each occurrence is independently hydrogen or C₁₋₄ alkyl (preferably, methyl). In some embodiments, one or two instances of X is O, one X is triazole, and all other instances of X is CR₂, wherein R at each occurrence is independently hydrogen or C₁₋₄ alkyl (preferably, methyl). In some embodiments, R is hydrogen. The definition and preferred definition of G¹, G², G⁴, r, and A⁻ are also described herein, including any of those corresponding definitions shown in compounds according to any of the compounds listed in Table 1 herein or any of the compound according to Examples 1-221.

### Formula III and III-B

In some embodiments, the present disclosure provides a compound of Formula III or III-B, or a pharmaceutically acceptable salt or ester thereof:

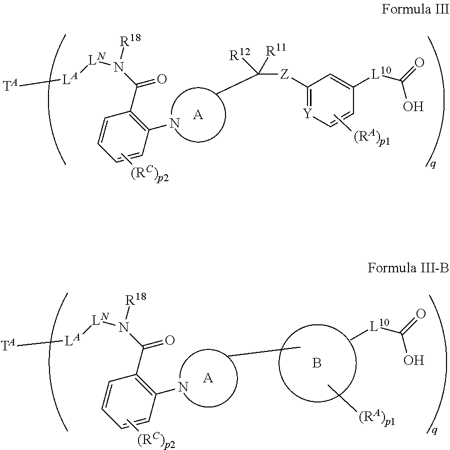

wherein:
Y is CH, CR^{A}, or N;
Z is O, S, NH, or N(C₁₋₄ alkyl);
R¹¹ and R¹² are each independently hydrogen or C₁₋₄ alkyl;
Ring A is an optionally substituted 4-12 membered nitrogen-containing ring;
Ring B is an optionally substituted monocyclic heteroaryl or a bicyclic aryl or heteroaryl ring, such as a benzofuran ring;
L^{N} is null, an optionally substituted C₁₋₆ alkylene, or an optionally substituted C₁₋₆ heteroalkylene having 1-3 heteroatoms;
L¹⁰ is an alkylene (e.g., a C₁₋₆ alkyl alkylene), optionally substituted with 1-3 substituents independently selected from halogen, optionally substituted C₁₋₆ alkyl, optionally substituted C₂₋₆ alkenyl, optionally substituted C₂₋₆ alkynyl, optionally substituted C₁₋₆ heteroalkyl, optionally substituted C₃₋₆ cycloalkyl, optionally substituted C₁₋₆ alkoxy, optionally substituted C₃₋₆ cycloalkoxy, optionally substituted heterocyclyl, optionally substituted aryl, and optionally substituted heteroaryl, or two substituents are joined to form an optionally substituted ring structure;
R^{A} at each occurrence is independently halogen, CN, optionally substituted C₁₋₆ alkyl, optionally substituted C₃₋₆ cycloalkyl, optionally substituted C₁₋₆ alkoxy, or optionally substituted C₃₋₆ cycloalkoxy, or two R^{A} are joined to form an optionally substituted ring structure; p1 is 0, 1, or 2;
R^{C} at each occurrence is independently halogen, CN, optionally substituted C₁₋₆ alkyl, optionally substituted C₃₋₆ cycloalkyl, optionally substituted C₁₋₆ alkoxy, or optionally substituted C₃₋₆ cycloalkoxy, or two R^{C} are joined to form an optionally substituted ring structure; p2 is 0, 1, 2, or 3;
R¹⁸ is an optionally substituted phenyl or an optionally substituted heteroaryl, and wherein:
q is an integer of 1-10, preferably, 1 or 2, and
T^{A} is a hydrophilic group and L^{A} is a linker. In preferred embodiments,
T^{A} is a hydrophilic group having a terminal atom(s) selected from N, O, S, or C, which is covalently bonded with a first end atom of L^{A}, wherein (1) when the terminal atom(s) is N of a basic amine group, then the corresponding compound T^{A}-(C(O)—CH₃)_{q} has a cLogP of less than 1, wherein the —C(O)—CH₃ is bonded with the terminal N atom(s); (2) when the terminal atom(s) is C of a C(O) group, then the corresponding compound T^{A}-(OH)_{q} has a cLogP of less than 1, wherein the —OH is bonded with the terminal C atom(s); (3) when the terminal atom(s) is S of a SO₂ group, then T^{A}-(OH)_{q} has a cLogP of less than 1, wherein the —OH is bonded with the terminal S atom(s); or (4) when (1)-(3) do not apply, then T^{A}-H_{q} has a cLogP of less than 1; and
L^{A} is a linker characterized in that the maximum length between the two end atoms of L^{A} is at least the maximum length between the two end carbon atoms of —(CH₂)₁₀—, wherein (1) only one end atom of L^{A} is C of a C(O) or S of a SO₂ group, which is bonded with T^{A}, and the corresponding compound H-L^{A}-OH has a cLogP of at least 3, wherein the —OH is bonded with the end C(O) or SO₂ group; or (2) neither end atoms of L^{A} is C of a C(O) or S of a SO₂ group, and the corresponding compound H-L^{A}-H has a cLogP of at least 3.

In some embodiments, the compound has a structure according to Formula III.

In some embodiments, the compound has a structure according to Formula III-B.

In some embodiments according to Formula III or III-B, L^{N} is null, i.e., L^{A} is directly connected to the amide nitrogen atom shown in Formula III or III-B. To be clear, for the purposes herein, the definition of L^{N} as null should not be interpreted such that in such embodiments, L^{A} cannot contain a fragment that fits into one or more of the definitions of L^{N} described herein. Rather, in embodiments, when L^{N} is defined as null, the variable L^{A} can have any of the definitions herein described for L^{N}-L^{A} in embodiments where L^{N} is not null.

In some embodiments according to Formula III or III-B, L^{N} is a branched or straight chained C₁₋₆ alkyl alkylene, such as

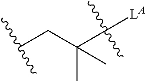

(L^{A} is shown to show direction of connection). In some embodiments according to Formula III or III-B, L^{N} is

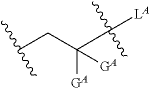

(L^{A} is shown to show direction of connection), wherein G^{A} at each occurrence is independently hydrogen or an optionally substituted C₁₋₄ alkyl, or two G^{A} are joined to form a 3-6 membered ring, such as a cyclopropyl or cyclobutyl ring. In some preferred embodiments, G^{A} at each occurrence is methyl.

In some embodiments, q in Formula III or III-B is 1, and the compound of Formula III can be characterized as having a Formula III-1 or III-B-1:

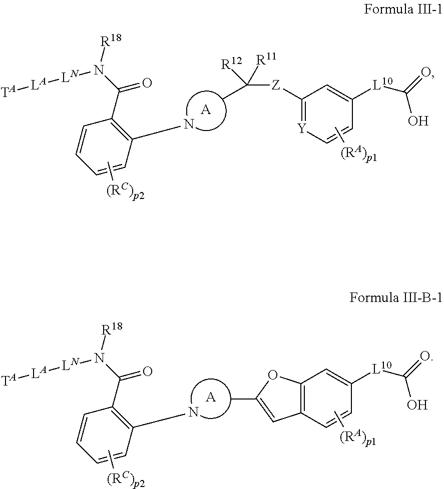

wherein the variables T^{A}, L^{A}, L^{N}, L¹⁰, R¹¹, R¹², R¹⁸, R^{A}, R^{C}, ring A, Y, Z, p1, and p2 are defined herein, which include any of those described and preferred herein in any combinations.

### Linker L^{A}

As with Formula I and II discussed herein, one factor that can determine whether the compound of Formula III or III-B can be a potent GPR40 agonist is the length of the linker (e.g., L^{A}) but not the exact chemical structure of the linker. As shown in the Examples section herein, when the length of the linker is below certain threshold, the EC50 value can increase significantly. Accordingly, the present inventors envision that the maximum length between the two end atoms of linker L^{A} in Formula III or III-B should be at least that between the two end carbon atoms of an alkyelene chain —(CH₂)₁₀—, similar to those described hereinabove in connection with Formula I. In other words, the longest chain length of L^{A} should be equal to or greater than the longest chain length of the alkyelene chain —(CH₂)₁₀—. The meaning and determination of the maximum distance between the two end non-hydrogen atoms of linker L^{A} as discussed above for Formula I or II also apply to Formula III. In some embodiments, L^{A} can be characterized in that the maximum length between the two end atoms of L^{A} is at least that between the two end carbon atoms of —(CH₂)₁₂—, preferably, at least that of —(CH₂)₁₄—, more preferably, at least that of —(CH₂)₁₆—. In some embodiments, L^{A} can be characterized in that the maximum length between the two end atoms of L^{A} is between (i) the maximum length between the two end carbon atoms of —(CH₂)₁₂— and (ii) the maximum length between the two end carbon atoms of —(CH₂)₅₀—.

In some embodiments, in Formula III (e.g., Formula III-1) or III-B (e.g., Formula III-B-1), L^{A} can be represented by a formula of (X) m, wherein X at each occurrence is independently CR₂, C(═O), —C(R)═C(R)—,

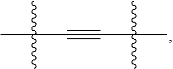 [SMILES: *C#C*]

SiR₂, O, S, SO₂, NR, [NR₂]⁺, or a ring structure, preferably 3-10 membered ring structure, wherein R at each occurrence is independently hydrogen, halogen, optionally substituted C₁₋₄ alkyl, or optionally substituted C₁₋₄ alkoxy, typically hydrogen or C₁₋₄ alkyl, and the integer m is at least 10, such as at least 12, at least 14, at least 16, at least 18, at least 20, at least 50, such as 12-50, 16-50, etc. As one end X group is bonded with L^{N}(when L^{N} is null, it should be understood that the covalent bond is formed with an amide nitrogen atom in Formula III or III-B), the X group of that end is preferably CR₂ or a ring structure, preferably 3-10 membered ring structure as described herein, more preferably, CR₂, such as CH₂. Thus, in any of the embodiments described herein in connection with Formula III or III-B, unless otherwise specified or contrary from context, the end X group that is bonded with L^{N}(when L^{N} is null, it should be understood that the covalent bond is formed with the amide nitrogen atom in Formula III (e.g., Formula III-1) or III-B (e.g., Formula III-B-1)) can be CR₂, such as CH₂. Suitable X groups for L^{A} in Formula III or III-B (e.g., Formula III-B-1) also include any of those described and preferred herein in connection with Formula I or II. Typically, in L^{A}, 0, 1, or 2 instances of X representing a ring structure, preferably 3-10 membered ring structure (such as a C₃₋₆ cycloalkyl such as cyclopropyl, a 5 or 6 membered heteroaryl, such as a triazole ring). In some embodiments, two consecutive X can be —C(O)O— or —C(O) NR—. In some embodiments, one instance of X can be a cyclopropane, cyclobutane or bicyclobutane[1.1] ring. In some embodiments, one instance of X can be a 5 or 6-membered heteroaryl, such as a triazole ring. In some embodiments, one instance of X can be a cyclopentane, cyclohexane or cycloheptane ring. Typically, one of the end X group connects to T^{A} through a carbon atom. The total number of non-hydrogen atoms of L^{A} can be typically between 10-100, such as 12-30, 14-50, 16-50, 18-100, etc.

Another factor that can determine whether the compound of Formula III or III-B can be a potent GPR40 agonist is the hydrophobicity of the linker (e.g., L^{A}) but not the exact chemical structure of the linker. In general, L^{A} should be a hydrophobic moiety. In some embodiments, only one end atom of L^{A} is C of a C(O) or S of a SO₂ group, in such embodiments, the hydrophobicity of L^{A} can be typically characterized in that the corresponding compound H-L^{A}-OH has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc., wherein the —OH is bonded with the end C(O) or SO₂ group. In some embodiments, neither of the two end atoms of L^{A} is C of a C(O) or S of a SO₂ group, in such embodiments, the hydrophobicity of L^{A} can be characterized in that the corresponding compound H-L^{A}-H has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc.

For example, in some embodiments, L^{A} is (X)ₘ₋₁—C(O)—, wherein the C(O) end is bonded with T^{A}, wherein X at each occurrence is independently CR₂, C(═O), —C(R)═C(R)—,

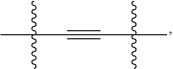 [SMILES: *C#C*]

SiR₂, O, S, SO₂, NR, [NR₂]⁺ or a ring structure, preferably 3-10 membered ring structure, provided that the end X group (i.e., the X group in L^{A} that is furthest away from the C(O) end) is not C(O) or SO₂, wherein R at each occurrence is independently hydrogen, halogen, optionally substituted C₁₋₄ alkyl, or optionally substituted C₁₋₄ alkoxy, typically hydrogen or C₁₋₄ alkyl, and the integer m is at least 10, such as at least 12, at least 14, at least 16, at least 18, at least 20, at least 50, such as 12-50, 16-50, etc., and the hydrophobicity of —(X) m-1—C(O)— is characterized in that the corresponding compound H—(X) m-1-COOH should have a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc. The “m-1” should be understood as the integer m minus 1, not to be misunderstood as a different designated variable.

In some embodiments, L^{A} is (X) m, wherein X at each occurrence is independently CR₂, C(═O), —C(R)═C(R)—,

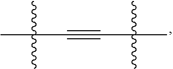 [SMILES: *C#C*]

SiR₂, O, S, SO₂, NR, [NR₂]⁺ or a ring structure, preferably 3-10 membered ring structure, provided that neither of the end X groups is C(O) or SO₂, wherein R at each occurrence is independently hydrogen, halogen, optionally substituted C₁₋₄ alkyl, or optionally substituted C₁₋₄ alkoxy, typically hydrogen or C₁₋₄ alkyl, and the integer m is at least 10, such as at least 12, at least 14, at least 16, at least 18, at least 20, at least 50, such as 12-50, 16-50, etc., then the corresponding compound H—(X)ₘ—H should have a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc.

In some embodiments, L^{A} is -X12-30—(e.g., -X14-, -X16-, -X18-, -X20-, -X24-, -X14-30-, -X16-30-, -X18-30-, etc.), wherein X at each occurrence is independently CR₂, C(═O), —C(R)═C(R)—,

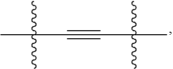 [SMILES: *C#C*]

SiR₂, O, S, SO₂, NR, [NR₂]⁺, or a ring structure, preferably 3-10 membered ring structure, provided that neither of the end X groups is C(O) or SO₂; wherein R at each occurrence is independently hydrogen, halogen, optionally substituted C₁₋₄ alkyl, or optionally substituted C₁₋₄ alkoxy, typically hydrogen or C₁₋₄ alkyl, and wherein the hydrophobicity of L^{A} can be characterized in that the corresponding compound H-L^{A}-H has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc. In some embodiments, the total number of non-hydrogen atoms of L^{A} is between 12-100, such as 12, 14, 16, 18, 20, 24, 30, 40, 50, 100, or any ranges between the recited values, such as 12-30, 14-50, 16-50, 18-100, etc. In some embodiments, two consecutive X can represent-C(O)O— or —C(O) NR—. In some embodiments, one or more (e.g., 1 or 2) instances of X can be a ring structure selected from cyclopropane, cyclobutane, bicyclobutane [1.1], cyclopentane, cyclohexane or cycloheptane. In some embodiments, one or more (e.g., 1) instances of X can be a ring structure selected from phenyl or 5 or 6-membered heteroaryl, such as triazole. In some embodiments, R is hydrogen.

In some embodiments, L^{A} is -X12-30—C(O)—, wherein the C(O) is directly bonded with T^{A}, and wherein X at each occurrence is independently CR₂, C(═O), —C(R)═C(R)—,

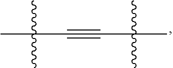 [SMILES: *C#C*]

SiR₂, O, S, SO₂, NR, [NR₂]⁺, or a ring structure, preferably 3-10 membered ring structure, provided that the end X group (i.e., the X group in L^{A} that is furthest away from the C(O) end) is CR₂ or a ring structure, preferably 3-10 membered ring structure; wherein R at each occurrence is independently hydrogen, halogen, optionally substituted C₁₋₄ alkyl, or optionally substituted C₁₋₄ alkoxy, typically hydrogen or C₁₋₄ alkyl, and wherein the hydrophobicity of L^{A} can be characterized in that the corresponding compound H-X12-30-C(O)—OH has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc. In some embodiments, the total number of non-hydrogen atoms of L^{A} is between 14-100, such as 14, 16, 18, 20, 24, 30, 40, 50, 100, or any ranges between the recited values, such as 14-30, 14-50, 16-50, 18-100, etc. In some embodiments, two consecutive X can be —C(O)O— or —C(O) NR—. In some embodiments, one or more instances (e.g., 1 or 2) of X can be a ring structure selected from cyclopropane, cyclobutane, bicyclobutane [1.1], cyclopentane, cyclohexane or cycloheptane. In some embodiments, one or more instances (e.g., 1) of X can be a ring structure selected from phenyl or 5 or 6-membered heteroaryl, such as triazole. In some embodiments, R is hydrogen.

In some preferred embodiments, L^{A} is —C₁₂₋₃₀ alkylene- or —C₁₂₋₃₀ alkylene-C(O)—, wherein the —C₁₂₋₃₀ alkylene- is optionally substituted, wherein the optional substituents can optionally be joined together to form a double bond, triple bond, or a ring structure (typically a 3-10 membered ring structure), wherein the end carbon atoms of the —C₁₂₋₃₀ alkylene- are not substituted with oxo (═O), wherein the longest chain length of L^{A} is at least that of —(CH₂)₁₂—, such as at least that of —(CH₂)₁₄—, at least that of —(CH₂)₁₆—, at least that of —(CH₂)₁₈—, and wherein the hydrophobicity of L^{A} can be characterized in that the corresponding compound H—C₁₂₋₃₀ alkylene-H or H—C₁₂₋₃₀ alkylene-C(O)—OH has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc. In some embodiments, the total number of non-hydrogen atoms of L^{A} is between 14-100, such as 14, 16, 18, 20, 24, 30, 40, 50, 100, or any ranges between the recited values, such as 14-30, 14-50, 16-50, 18-100, etc. In some embodiments, L^{A} is unsubstituted-C12-30 alkylene-, such as a linear or branched C₁₂₋₃₀ alkylene. In some embodiments, L^{A} is unsubstituted-C₁₂₋₃₀ alkylene-C(O)—, wherein the C₁₂₋₃₀ alkylene can be linear or branched. In some preferred embodiments, L^{A} is —C₁₂₋₃₀ alkylene- or —C₁₂₋₃₀ alkylene-C(O)—, wherein the C₁₂₋₃₀ alkylene is a linear and unsubstituted-C₁₂₋₃₀ alkylene-.

In some preferred embodiments, L^{A} or L^{N}-L^{A} in Formula III (e.g., Formula III-1) or III-B (e.g., Formula III-B-1) can be selected from a structure of:

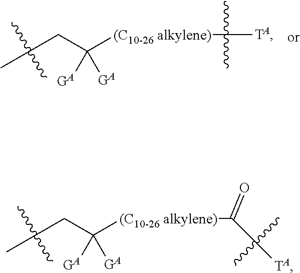

wherein the C₁₀₋₂₆ alkylene is optionally substituted, and wherein G^{A} at each occurrence is independently hydrogen or an optionally substituted C₁₋₄ alkyl, or two G^{A} are joined to form a 3-6 membered ring, such as a cyclopropyl or cyclobutyl ring. In some preferred embodiments, G^{A} at each occurrence is methyl. In some embodiments, the C₁₀₋₂₆ alkylene is unsubstituted, such as a linear or branched C₁₀₋₂₆ alkylene. In some preferred embodiments, the C₁₀₋₂₆ alkylene is a linear and unsubstituted C₁₀₋₂₆ alkylene.

In some embodiments, L^{A} is a 12-30 membered heteroalkylene or -(12-30 membered heteroalkylene)-C(O)—, wherein the 12-30 membered heteroalkylene is optionally substituted and contains 1-6 heteroatoms independently selected from O, N, and S, wherein the sulfur atom(s), if present, is optionally oxidized, wherein the optional substituents can optionally be joined together to form a double bond, triple bond, or a ring structure (typically a 3-10 membered ring structure), wherein the end atom of the 12-30 membered heteroalkylene that forms a covalent bond with L^{N}(when L^{N} is null, it should be understood that the covalent bond is formed with the amide nitrogen atom in Formula III or III-B) is a carbon atom of a non-carbonyl group, wherein the longest chain length of L^{A} is at least that of —(CH₂)₁₂—, such as at least that of —(CH₂)₁₄—, at least that of —(CH₂)₁₆—, at least that of —(CH₂)₁₈—, and wherein the hydrophobicity of L^{A} can be characterized in that the corresponding compound H-(12-30 membered heteroalkylene)-H or H-(12-30 membered heteroalkylene)-C(O)—OH has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc. In some embodiments, the total number of non-hydrogen atoms of L^{A} is between 14-100, such as 14, 16, 18, 20, 24, 30, 40, 50, 100, or any ranges between the recited values, such as 14-30, 14-50, 16-50, 18-100, etc. In some embodiments, L^{A} is unsubstituted 12-30 membered heteroalkylene, such as a linear or branched 12-30 membered heteroalkylene. In some embodiments, L^{A} is unsubstituted-(12-30 membered heteroalkylene)-C(O)—, wherein the 12-30 membered heteroalkylene can be linear or branched. In some preferred embodiments, L^{A} is 12-30 membered heteroalkylene or -(12-30 membered heteroalkylene)-C(O)—, wherein the 12-30 membered heteroalkylene is a linear and unsubstituted. In some embodiments, the 12-30 membered heteroalkylene includes 1, 2, 3, 4, or 5 heteroatoms independently selected from O, S, and N.

In some preferred embodiments, L^{A} can be characterized as having a structure according to

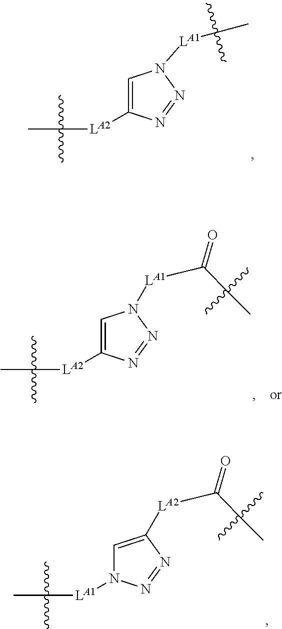

wherein the carbonyl is directly bonded with T^{A}, wherein L^{A1} and L^{A2} are each independently a bond, an optionally substituted —C₁₋₃₀ alkylene-, or an optionally substituted —C₁₋₃₀ heteroalkylene-containing 1-6 heteroatoms independently selected from O, N, and S, wherein the sulfur atom(s), if present, is optionally oxidized, wherein the optional substituents can optionally be joined together to form a double bond, triple bond, or a ring structure, wherein the end atom of the L^{A1} or L^{A2} that forms a covalent bond with T^{A} is not C of a C(O) or S of a SO₂ group, and the end atom of the L^{A1} or L^{A2} that forms a covalent bond with L^{N}(when L^{N} is null, it should be understood that the covalent bond is formed with the amide nitrogen atom in Formula III or III-B) is a carbon atom of a non-carbonyl group, wherein the longest chain length of L^{A} is at least that of —(CH₂)₁₂—, such as at least that of —(CH₂)₁₄—, at least that of —(CH₂)₁₆—, at least that of —(CH₂)₁₈—, and wherein the hydrophobicity of L^{A} can be characterized in that the corresponding compound

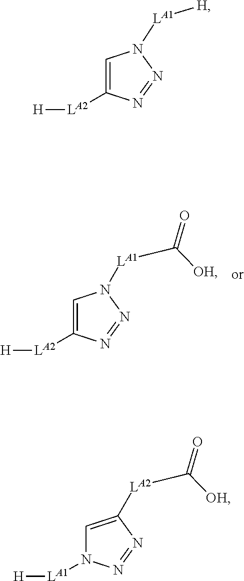

has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc. In some embodiments, the total number of non-hydrogen atoms of L^{A} is between 14-100, such as 14, 16, 18, 20, 24, 30, 40, 50, 100, or any ranges between the recited values, such as 14-30, 14-50, 16-50, 18-100, etc. In some embodiments, L^{A1} is a C₁₋₂₀ alkylene, such as a linear alkylene, (CH₂)₁₋₂₀. In some embodiments, L^{A1} is a C₁₋₁₀ heteroalkylene having 1-3 oxygen atoms. In some embodiments, L^{A2} is a bond. In some embodiments, L^{A2} is a C₁₋₂₀ alkylene, such as a linear alkylene, (CH₂)₁₋₂₀. In some embodiments, L^{A2} is a C₁₋₁₀ heteroalkylene having 1-3 oxygen atoms, such as —O—(C₁₋₁₀ alkylene)-, etc.

In some embodiments, L^{A} can be characterized as having a structure according to

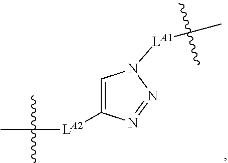

wherein L^{A1} and L^{A2} are each independently a bond, —C₁₋₃₀ alkylene-, or —C₁₋₃₀ heteroalkylene-containing 1-6 heteroatoms independently selected from O, N, and S, wherein the sulfur atom(s), if present, is optionally oxidized, wherein the end atom of the L^{A1} or L^{A2} that form a covalent bond with T^{A} is not C of a C(O) or S of a SO₂ group, and the end atom of the L^{A1} or L^{A2} that forms a covalent bond with L^{N}, when L^{N} is null, it should be understood that the covalent bond is formed with the amide nitrogen atom in Formula III or III-B, is a carbon atom of a non-carbonyl group, wherein the total number of non-hydrogen atoms of L^{A} is between 15-50, such as 18, 20, 25, 30, 35, 40, 45, or 50, or any ranges between the recited value, wherein the longest chain length of L^{A} is at least that of (CH₂)₁₂, such as at least that of —(CH₂)₁₄—, at least that of —(CH₂)₁₆, at least that of (CH₂)₁₈, wherein the hydrophobicity of L^{A} can be characterized in that the corresponding compound H-L^{A}-H has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc. Preferably, L^{A1} is not a bond. However, when L^{A1} is a bond, the triazole nitrogen atom is directly directly bonded with T^{A}. In some embodiments, L^{A2} is a bond. In some embodiments, L^{A1} is a C₁₋₂₀ alkylene, such as a linear alkylene, (CH₂)₁₋₂₀. In some embodiments, L^{A1} is a C₁₋₁₀ heteroalkylene having 1-3 oxygen atoms. In some embodiments, L^{A2} is a C₁₋₂₀ alkylene, such as a linear alkylene, (CH₂)₁₋₂₀. In some embodiments, L^{A2} is a C₁₋₁₀ heteroalkylene having 1-3 oxygen atoms, such as —O—(C₁₋₁₀ alkylene)-, etc.

In some embodiments, L^{A} can be characterized as having a structure according to

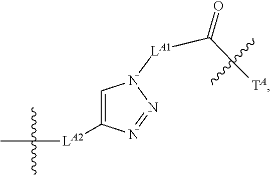

wherein L^{A1} and L^{A2} are each independently a bond, —C₁₋₃₀ alkylene-, or —C₁₋₃₀ heteroalkylene-containing 1-6 heteroatoms independently selected from O, N, and S, wherein the sulfur atom(s), if present, is optionally oxidized, wherein the end atom of L^{A2} that forms a bond with L^{N}, when L^{N} is null, it should be understood that the covalent bond is formed with the amide nitrogen atom, is a carbon atom of a non-carbonyl group, wherein the total number of non-hydrogen atoms of L^{A} is between 15-50, such as 18, 20, 25, 30, 35, 40, 45, or 50, or any ranges between the recited value, wherein the longest chain length of L^{A} is at least that of —(CH₂)₁₂—, such as at least that of —(CH₂)₁₄—, at least that of —(CH₂)₁₆—, at least that of —(CH₂)₁₈—, wherein the hydrophobicity of L^{A} can be characterized in that the corresponding compound

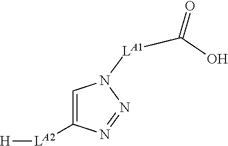

has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc. Preferably, L^{A1} is not a bond. In some embodiments, L^{A2} is a bond. In some embodiments, L^{A1} is a C₁₋₂₀ alkylene, such as a linear alkylene, (CH₂)₁₋₂₀. In some embodiments, L^{A1} is a C₁₋₁₀ heteroalkylene having 1-3 oxygen atoms. In some embodiments, L^{A2} is a C₁₋₂₀ alkylene, such as a linear alkylene, (CH₂)₁₋₂₀. In some embodiments, L^{A2} is a C₁₋₁₀ heteroalkylene having 1-3 oxygen atoms, etc.

In some embodiments, L^{A} can be characterized as having a structure according to

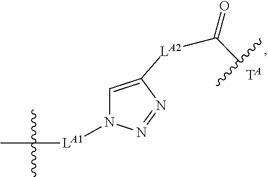

wherein L^{A1} and L^{A2} are each independently a bond, —C₁₋₃₀ alkylene-, or —C₁₋₃₀ heteroalkylene-containing 1-6 heteroatoms independently selected from O, N, and S, wherein the sulfur atom(s), if present, is optionally oxidized, wherein the end atom of L^{A1} that forms a bond with L^{N}, when L^{N} is null, it should be understood that the covalent bond is formed with the amide nitrogen atom, is a carbon atom of a non-carbonyl group, wherein the total number of non-hydrogen atoms of L^{A} is between 15-50, such as 18, 20, 25, 30, 35, 40, 45, or 50, or any ranges between the recited value, wherein the longest chain length of L^{A} is at least that of —(CH₂)₁₂—, such as at least that of —(CH₂)₁₄—, at least that of —(CH₂)₁₆—, at least that of —(CH₂)₁₈—, wherein the hydrophobicity of L^{A} can be characterized in that the corresponding compound

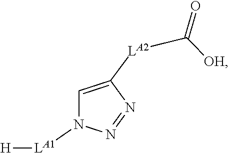

has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc. Preferably, L^{A1} is not a bond. In some embodiments, L^{A2} is a bond. In some embodiments, L^{A1} is a C₁₋₂₀ alkylene, such as a linear alkylene, (CH₂)₁₋₂₀. In some embodiments, L^{A1} is a C₁₋₁₀ heteroalkylene having 1-3 oxygen atoms. In some embodiments, L^{A2} is a C₁₋₂₀ alkylene, such as a linear alkylene, (CH₂)₁₋₂₀. In some embodiments, L^{A2} is a C₁₋₁₀ heteroalkylene having 1-3 oxygen atoms, such as —O—(C₁₋₁₀ alkylene)-, etc.

In some preferred embodiments, L^{A} or L^{N}-L^{A} in Formula III (e.g., Formula III-1) or III-B (e.g., Formula III-B-1) can be selected from a structure of:

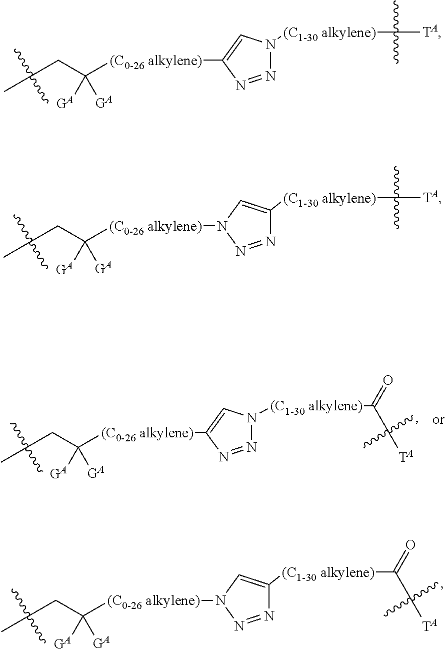

wherein the C₀₋₂₆ alkylene or C₁₋₃₀ alkylene is optionally substituted, and wherein G^{A} at each occurrence is independently hydrogen or an optionally substituted C₁₋₄ alkyl, or two G^{A} are joined to form a 3-6 membered ring, such as a cyclopropyl or cyclobutyl ring. In some preferred embodiments, G^{A} at each occurrence is methyl. In some embodiments, the C₀₋₂₆ alkylene or C₁₋₃₀ alkylene is unsubstituted, which is a linear or branched alkylene. In some embodiments, the C₀₋₂₆ alkylene or C₁₋₃₀ alkylene is linear and unsubstituted alkylene.

In some preferred embodiments, L^{A} or L^{N}-L^{A} in Formula III (e.g., Formula III-1) or III-B (e.g., Formula III-B-1) can be selected from a structure of:

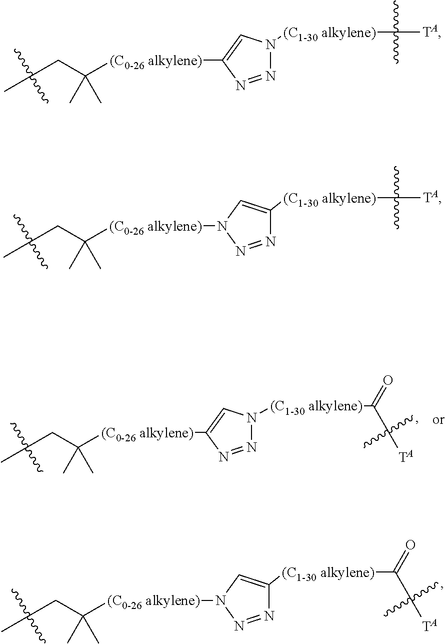

wherein the C₀₋₂₆ alkylene or C₁₋₃₀ alkylene is optionally substituted. In some embodiments, the C₀₋₂₆ alkylene or C₁₋₃₀ alkylene is unsubstituted, which is a linear or branched alkylene. In some embodiments, the C₀₋₂₆ alkylene or C₁₋₃₀ alkylene is linear and unsubstituted alkylene.

### Hydrophilic Group T^{A}

As with compounds of Formula I and II, another factor that can determine whether the compound of Formula III or III-B can be a potent GPR40 agonist is the hydrophilicity or polarity of T^{A}, but the exact chemical structure of T^{A} is not as important. Suitable and preferred T^{A} for Formula III or III-B include any of those described and preferred in connection with Formula I or II. In Formula III or III-B, T^{A} is a hydrophilic group having a terminal atom(s) selected from N, O, S, or C, which is covalently bonded with an end atom such as carbon or nitrogen atom(s) of L^{A}, wherein (1) when the terminal atom(s) is N of a basic amine group, then the corresponding compound T^{A}-(C(O)—CH₃)_{q} has a cLogP of less than 1, wherein the —C(O)—CH₃ is bonded with the terminal N atom(s); (2) when the terminal atom(s) is C of a C(O) group, then the corresponding compound T^{A}-(OH)_{q} has a cLogP of less than 1, wherein the —OH is bonded with the terminal C atom(s); (3) when the terminal atom(s) is S of a SO₂ group, then the corresponding compound T^{A}-(OH)_{q} has a cLogP of less than 1, wherein the —OH is bonded with the terminal S atom(s); or (4) when (1)-(3) do not apply, then T^{A}-H_{q} has a cLogP of less than 1. In some embodiments, the terminal atom(s) is N of a basic amine group, and T^{A} is characterized in that the corresponding compound T^{A}-(C(O)—CH₃)_{q} has a cLogP of less than 0 (e.g., less than −1, less than −2, less than −3, less than-3.5, less than −4, or even lower). In some embodiments, the terminal atom(s) is C of a C(O) group, and T^{A} is characterized in that the corresponding compound T^{A}-(OH)_{q} has a cLogP of less than 0 (e.g., less than −1, less than −2, less than −3, less than −3.5, less than −4, or even lower). In some embodiments, the terminal atom(s) is S in a SO₂ group, and T^{A} is characterized in that the corresponding compound T^{A}-(OH), has a cLogP of less than 0 (e.g., less than −1, less than −2, less than −3, less than −3.5, less than −4, or even lower). In some embodiments, the terminal atom(s) is not N of a basic amine group, C of a C(O) group, or S in a SO₂ group, and T^{A} is characterized in that the corresponding compound T^{A}-H_{q} has a cLogP of less than 0 (e.g., less than −1, less than −2, less than −3, less than −3.5, less than −4, or even lower).

In some preferred embodiments, T^{A} can contain a charged group, including positively charged, such as containing a quaternary nitrogen atom, negatively charged, such as SO₃⁻, or containing a zwitterion structure. When T^{A} contains a charged group, it should be understood that a counterion, preferably, a pharmaceutically acceptable anion or cation, if necessary, exists to balance the charges so that the compound of Formula III or III-B is overall neutral.

In some embodiments, T^{A} is characterized as having a charged group (including zwitterion structures) or a group that can become charged at pH 7, such as primary amine, secondary amine, tertiary amine, quaternary amine, carboxylic acid, etc.

In some embodiments, T^{A} is characterized as having at least two hydrogen bond donors.

In some embodiments, T^{A} is characterized as having at least two hydrogen bond acceptors.

In some embodiments, T^{A} is T^{A1}, T^{A1}-L^{B}-, T^{A1}-L^{B}-(heteroalkylene), or T^{A1}-L^{B}-(heteroalkylene)-L^{B}-, wherein L^{B} at each occurrence is independently —SO₂—, —C(═O)—, or a moiety selected from:

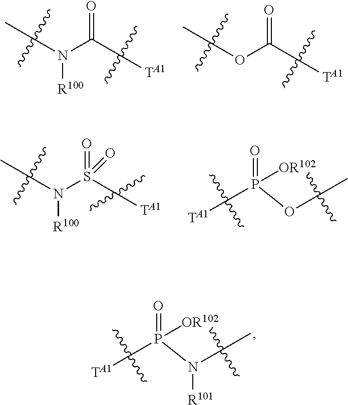

wherein R¹⁰⁰, R¹⁰¹ and R¹⁰² at each occurrence is independently hydrogen, optionally substituted alkyl, or optionally substituted cycloalkyl, wherein T^{A1} is characterized as having a structure of:

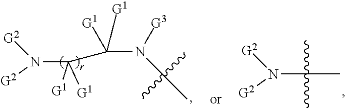

wherein G¹ at each occurrence is independently hydrogen, halogen, optionally substituted C₁₋₄ alkyl, or optionally substituted C₁₋₄ heteroalkyl (such as optionally substituted C₁₋₄ alkoxy), or two gem G¹ group can join to form an ═O, ═NH, or ═N(C₁₋₄ alkyl), or two or more G¹ groups can join to form a ring structure, the integer r is 1-10, such as 1 or 2,
G² at each occurrence is independently hydrogen, G¹⁰, C(O)-G¹⁰, SO₂G¹⁰, C(O)—NH-G¹⁰, or

SO₂NHG¹⁰, C(O)—O-G¹⁰, or SO₂OG¹⁰, wherein G¹⁰ at each occurrence is independently an optionally substituted alkyl, an optionally substituted heteroalkyl, or an optionally substituted 3-10 membered ring structure, which can be carbocyclic, heterocyclic, aromatic, heteroaromatic, or a combination thereof,
G³ is hydrogen or an optionally substituted alkyl (e.g., C₁₋₄ alkyl);
or two G² or one G² and one G¹ or one G¹ and G³ can be joined to form a ring structure such as a 3-10 membered ring structure,
wherein the fragment

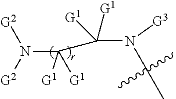

is further characterized in that (i) when the CG¹G¹ group next to the NG³ is not C(═O), the corresponding compound

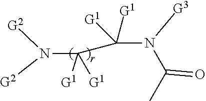

has a cLogP less than 1, preferably, less than 0 (e.g., less than −1, less than −2, less than −3, less than −3.5, less than −4, or even lower), or (ii) when the CG¹G¹ group next to the NG³ is C(═O), the corresponding compound

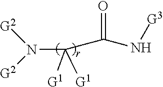

has a cLogP less than 1, preferably, less than 0 (e.g., less than −1, less than −2, less than −3, less than −3.5, less than −4, or even lower), and

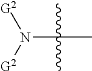

wherein the fragment is further characterized in that (i) when the nitrogen is a basic nitrogen, the corresponding compound (G²)₂N—C(O)—CH₃ has a cLogP less than 1, preferably, less than 0 (e.g., less than −1, less than −2, less than −3, less than −3.5, less than −4, or even lower), or (ii) when the nitrogen is a nonbasic nitrogen, the corresponding compound (G²)₂N—H has a cLogP less than 1, preferably, less than 0 (e.g., less than −1, less than −2, less than −3, less than −3.5, less than −4, or even lower). In preferred embodiments, G³ is hydrogen. In some preferred embodiments, T^{A} is T^{A1} or T^{A1}-L^{B}-. In some embodiments, T^{A} is T^{A1}-L^{B}-(heteroalkylene) or T^{A1}-L^{B}-(heteroalkylene)-L^{B}-, wherein the heteroalkylene can be a glycol chain, such as a polyethylene glycol chain.

In some embodiments, T^{A} is T^{A1}. In some embodiments, T^{A} is T^{A1}—C(═O)—. In some embodiments, T^{A1} is characterized as having a structure of:

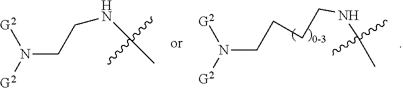

In some embodiments, T^{A1} is characterized as having a structure of:

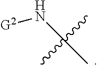

In some embodiments, G² at each occurrence independently can have a structure of

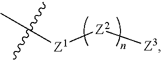

wherein n is an integer of 0-100, preferably, 0-10 (e.g., 0-4, 1-5 or 2-6 etc.), Z¹ is CH₂, C(O), SO, SO₂, CH(C₁₋₄ alkyl), C(C₁₋₄ alkyl)(C₁₋₄ alkyl) or 3-10 membered ring structure, Z² at each occurrence is independently CH₂, O, S, SO, SO₂, C(O), CH(C₁₋₄ alkyl), C(C₁₋₄ alkyl)(C₁₋₄ alkyl), NH, N(C₁₋₄ alkyl), [N(C₁₋₄ alkyl)(C₁₋₄ alkyl)]⁺ or 3-10 membered ring structure, and Z³ is H, C₁₋₄ alkyl, OH, SO₃H, COOH, NH₂, NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, SO₂NH₂, CONH(C₁₋₄ alkyl), CON(C₁₋₄ alkyl)(C₁₋₄ alkyl), SO₂NH(C₁₋₄ alkyl), or SO₂N(C₁₋₄ alkyl)(C₁₋₄ alkyl), wherein each of the C₁₋₄ alkyl is independently selected and optionally substituted, for example, with 1-3 G²⁰ groups defined herein. In some embodiments, none of the Z² is a 3-10 membered ring structure. In some embodiments, one or more instances of Z² is a 3-10 membered ring structure, such as a nitrogen containing ring, for example,

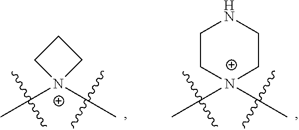 [SMILES: *[N+]1(*)CCC1 ; *[N+]1(*)CCNCC1]

etc. In some embodiments, the Z² directly connects to Z³ is a 3-10 membered ring structure, such as a nitrogen containing ring, for example,

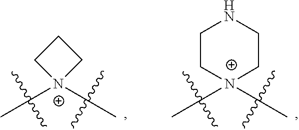 [SMILES: *[N+]1(*)CCC1 ; *[N+]1(*)CCNCC1]

etc. Preferably, at most 2 instances of Z² is a 3-10 membered ring structure. In some embodiments, G² at each occurrence is independently hydrogen, an optionally substituted C₁₋₆ alkyl or an optionally substituted C₁₋₆ heteroalkyl, for example, when substituted, the C₁₋₆ alkyl can be substituted with 1-3 substituents independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), and SO₂N(C₁₋₄ alkyl)(C1-4 alkyl), wherein each of the C₁₋₄ alkyl is independently selected and optionally substituted, for example, with 1-3 G²⁰ groups defined herein. The G²⁰ group herein at each occurrence can be independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), SO₂N(C₁₋₄ alkyl)(C₁₋₄ alkyl), or 3-10 membered ring structure, wherein each of the C₁₋₄ alkyl is independently selected and optionally substituted, for example, with 1-3 G²¹ groups defined herein. The G²¹ group herein at each occurrence can be independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ one or more instances of G² is C₁₋₄ alkyl, optionally substituted with COOH or an amide derivative, such as CONH—(C₁₋₄ alkylene)-NH₂ or CONH—(C₁₋₄ alkylene)-[N(C₁₋₄ alkyl)(C₁₋₄ alkyl)(C₁₋₄ alkyl)]⁺. For example, in some embodiments, one or two instances of G² is —(C₁₋₄ alkylene)-COOH, such as —CH₂COOH, and any remaining instance(s) of G² is C₁₋₄ alkyl. In some embodiments, one or two instances of G² is —(C₁₋₄ alkylene)-CONH—(C₁₋₄ alkylene)-NH₂, such as

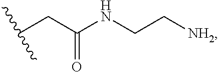 [SMILES: *CC(=O)NCCN]

and any remaining instance(s) of G² is C₁₋₄ alkyl. In some embodiments, one or two instances of G² is —(C₁₋₄ alkylene)-CONH—(C₁₋₄ alkylene)-[N(C₁₋₄ alkyl)(C₁₋₄ alkyl)(C₁₋₄ alkyl)]⁺, such as

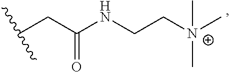 [SMILES: *CC(=O)NCC[N+](C)(C)C]

and any remaining instance(s) of G² is C₁₋₄ alkyl. In some embodiments, T^{A1} can be

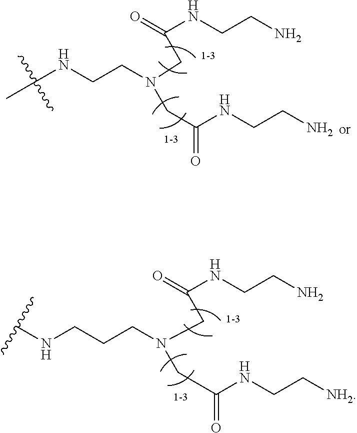

In some embodiments, T^{A1} can be

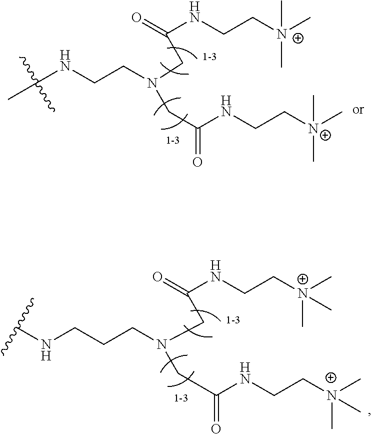

charge balanced with a counterion (e.g., described herein) as necessary. In some embodiments, T^{A1} can be

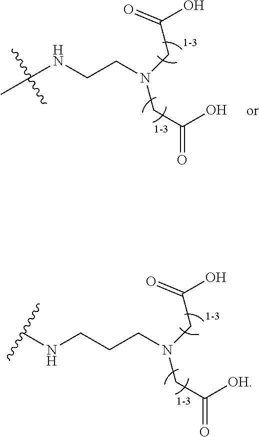

In some embodiments, T^{A1} can be

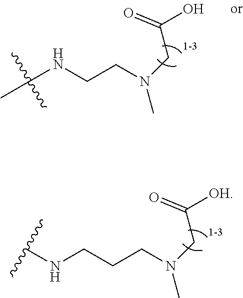

In some embodiments, T^{A1} can be

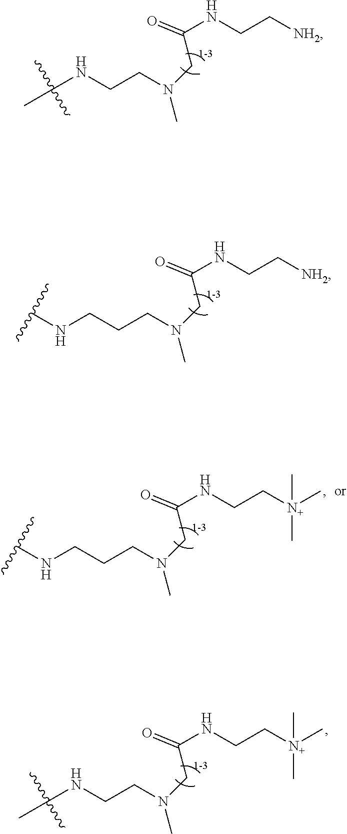

charge balanced with a counterion (e.g., described herein) as necessary. In some embodiments, T^{A1} can be

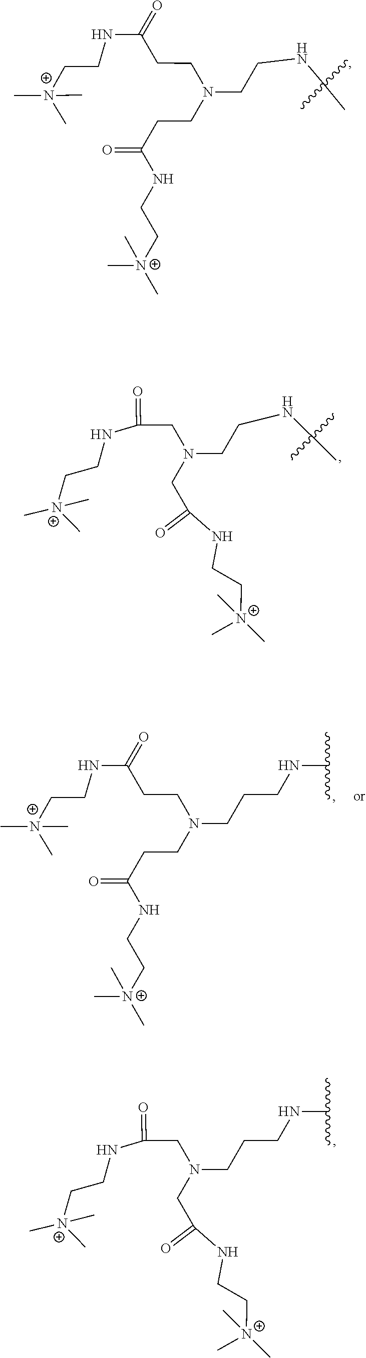 [SMILES: *NCCN(CCC(=O)NCC[N+](C)(C)C)CCC(=O)NCC[N+](C)(C)C ; *NCCN(CC(=O)NCC[N+](C)(C)C)CC(=O)NCC[N+](C)(C)C ; *NCCCN(CCC(=O)NCC[N+](C)(C)C)CCC(=O)NCC[N+](C)(C)C ; *NCCCN(CC(=O)NCC[N+](C)(C)C)CC(=O)NCC[N+](C)(C)C]

charge balanced with a counterion (e.g., described herein) as necessary. For example, in some embodiments, T^{A1} can be

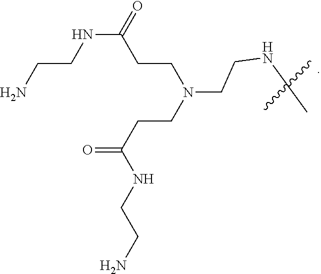 [SMILES: *NCCN(CCC(=O)NCCN)CCC(=O)NCCN]

In some embodiments, T^{A1} can be

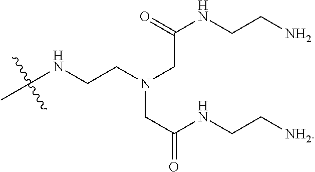 [SMILES: *NCCN(CC(=O)NCCN)CC(=O)NCCN]

In some embodiments, T^{A1} can be

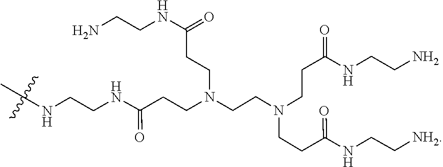 [SMILES: *NCCNC(=O)CCN(CCC(=O)NCCN)CCN(CCC(=O)NCCN)CCC(=O)NCCN]

In some embodiments, T^{A1} can be

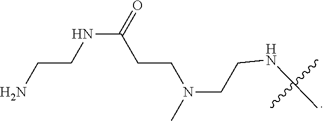 [SMILES: *NCCN(C)CCC(=O)NCCN]

In some embodiments, T^{A1} can be

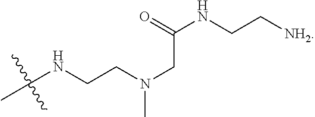 [SMILES: *NCCN(C)CC(=O)NCCN]

In some embodiments, T^{A1} can be

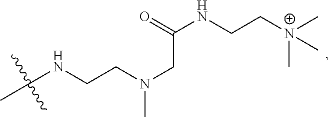 [SMILES: *NCCN(C)CC(=O)NCC[N+](C)(C)C]

charge balanced with a counterion (e.g., described herein) as necessary. In some embodiments, T^{A1} can be

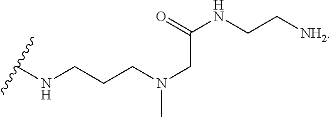 [SMILES: *NCCCN(C)CC(=O)NCCN]

In some embodiments, T^{A1} can be

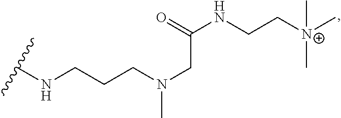 [SMILES: *NCCCN(C)CC(=O)NCC[N+](C)(C)C]

charge balanced with a counterion (e.g., described herein) as necessary. In some embodiments, T^{A1} can be

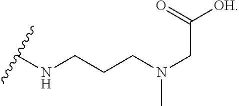 [SMILES: *NCCCN(C)CC(=O)O]

In some embodiments, T^{A} is T^{A1}, T^{A1}-L^{B}-, T^{A1}-L^{B}-(heteroalkylene), or T^{A1}-L^{B}-(heteroalkylene)-L^{B}-, wherein L^{B} at each occurrence is independently —SO₂—, —C(═O)—, or a moiety selected from:

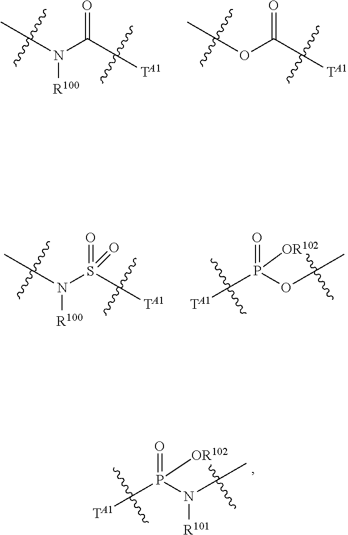

wherein R¹⁰⁰, R¹⁰¹ and R¹⁰² at each occurrence is independently hydrogen, optionally substituted alkyl, or optionally substituted cycloalkyl, wherein T^{A1} is characterized as having a structure of:

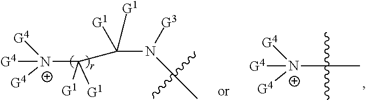

charge balanced with a counterion (e.g., described herein) as necessary, wherein G¹ at each occurrence is independently hydrogen, halogen, optionally substituted C₁₋₄ alkyl, or optionally substituted C₁₋₄ heteroalkyl (such as optionally substituted C₁₋₄ alkoxy), or two gem G¹ group can join to form an ═O, ═NH, or ═N(C₁₋₄ alkyl), or two or more G¹ groups can join to form a ring structure,
the integer r is 1-10, such as 1 or 2,
G³ is hydrogen or an optionally substituted alkyl (e.g., C₁₋₄ alkyl);
G⁴ at each occurrence is independently hydrogen or G¹⁰, wherein G¹⁰ at each occurrence is independently an optionally substituted alkyl, an optionally substituted heteroalkyl, or an optionally substituted 3-10 membered ring structure, which can be carbocyclic, heterocyclic, aromatic, heteroaromatic, or a combination thereof, or two G⁴ or one G⁴ and one G¹ or one G¹ and G³ can be joined to form an optionally substituted 3-10 membered ring structure,
wherein the fragment

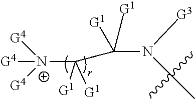

is characterized in that (i) when the CG¹G¹ group next to the NH is not C(═O), the corresponding compound

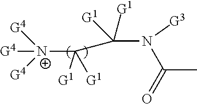

has a cLogP less than 1, preferably, less than 0 (e.g., less than −1, less than −2, less than −3, less than −3.5, less than −4, or even lower), or (ii) when the CG GI group next to the NH is C(═O), the corresponding compound

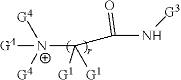

has a cLogP less than 1, preferably, less than 0 (e.g., less than −1, less than −2, less than −3, less than −3.5, less than −4, or even lower). In preferred embodiments, G³ is hydrogen. In some preferred embodiments, T^{A} is T^{A1} or T^{A1}-L^{B}-. In some embodiments, T^{A} is T^{A1}-L^{B}-(heteroalkylene) or T^{A1}-L^{B}-(heteroalkylene)-L^{B}-, wherein the heteroalkylene can be a glycol chain, such as a polyethylene glycol chain. In some embodiments, T^{A} is T^{A1}. In some embodiments, T^{A} is T^{A1}—C(═O)—. In some embodiments, T^{A1} is characterized as having a structure of:

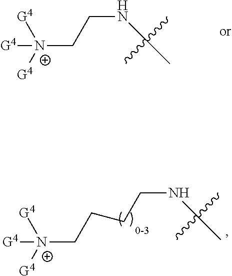

charge balanced with a counterion (e.g., described herein) as necessary. In some embodiments, T^{A1} is characterized as having a structure of:

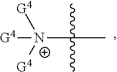

charge balanced with a counterion (e.g., described herein) as necessary. In some embodiments, G⁴ at each occurrence independently can have a structure of

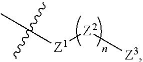

wherein n is an integer of 0-100, preferably, 0-10 (e.g., 0-4, 1-5 or 2-6 etc.), Z¹ is CH₂, C(O), SO₂, CH(C₁₋₄ alkyl), or C(C₁₋₄ alkyl)(C₁₋₄ alkyl), Z² at each occurrence is independently CH₂, O, S, SO, SO₂, C(O), CH(C₁₋₄ alkyl), C(C₁₋₄ alkyl)(C₁₋₄ alkyl), NH, N(C₁₋₄ alkyl), [N(C₁₋₄ alkyl)(C₁₋₄ alkyl)]⁺ or 3-10 membered ring structure, and Z³ is H, C₁₋₄ alkyl, OH, SO₃H, COOH, NH₂, NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, SO₂NH₂, CONH(C₁₋₄ alkyl), CON(C₁₋₄ alkyl)(C₁₋₄ alkyl), SO₂NH(C₁₋₄ alkyl), or SO₂N(C₁₋₄ alkyl)(C₁₋₄ alkyl), wherein each of the C₁₋₄ alkyl is independently selected and optionally substituted, for example, with 1-3 G²⁰ groups defined herein. Preferably, at most 2 instances of Z² is a 3-10 membered ring structure. In some embodiments, none of the Z² is a 3-10 membered ring structure. In some embodiments, one instance of Z² is a 3-10 membered ring structure, such as a nitrogen containing ring, for example,

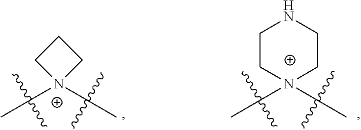

etc. In some embodiments, G⁴ at each occurrence is independently hydrogen, an optionally substituted C₁₋₆ alkyl or an optionally substituted C₁₋₆ heteroalkyl, for example, when substituted, the C₁₋₆ alkyl can be substituted with 1-3 substituents independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), and SO₂N(C₁₋₄ alkyl)(C1-4 alkyl), wherein each of the C₁₋₄ alkyl is independently selected and optionally substituted, for example, with 1-3 G²⁰ groups defined herein. The G²⁰ group herein at each occurrence can be independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), SO₂N(C₁₋₄ alkyl)(C₁₋₄ alkyl), or 3-10 membered ring structure, wherein each of the C₁₋₄ alkyl is independently selected and optionally substituted, for example, with 1-3 G²¹ groups defined herein. The G²¹ group herein at each occurrence can be independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ one or more instances of G⁴ is C₁₋₄ alkyl, such as methyl. In some embodiments, one or more instances of G⁴ is C₁₋₄ alkyl, optionally substituted with COOH or an amide derivative, such as CONH—(C₁₋₄ alkylene)-NH₂ or CONH—(C₁₋₄ alkylene)-[N(C₁₋₄ alkyl)(C₁₋₄ alkyl)(C₁₋₄ alkyl)]⁺. For example, in some embodiments, one or two instances of G⁴ is —(C₁₋₄ alkylene)-COOH, such as —CH₂COOH, and any remaining instance(s) of G⁴ is C₁₋₄ alkyl. In some embodiments, one or two instances of G⁴ is —(C₁₋₄ alkylene)-CONH—(C₁₋₄ alkylene)-NH₂, such as

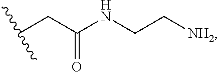 [SMILES: *CC(=O)NCCN]

and any remaining instance(s) of G⁴ is C₁₋₄ alkyl. In some embodiments, one or two instances of G⁴ is —(C₁₋₄ alkylene)-CONH—(C₁₋₄ alkylene)-[N(C₁₋₄ alkyl)(C₁₋₄ alkyl)(C₁₋₄ alkyl)]⁺, such as

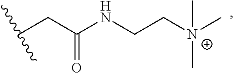 [SMILES: *CC(=O)NCC[N+](C)(C)C]

and any remaining instance(s) of G⁴ is C₁₋₄ alkyl. In some embodiments, T^{A1} can be

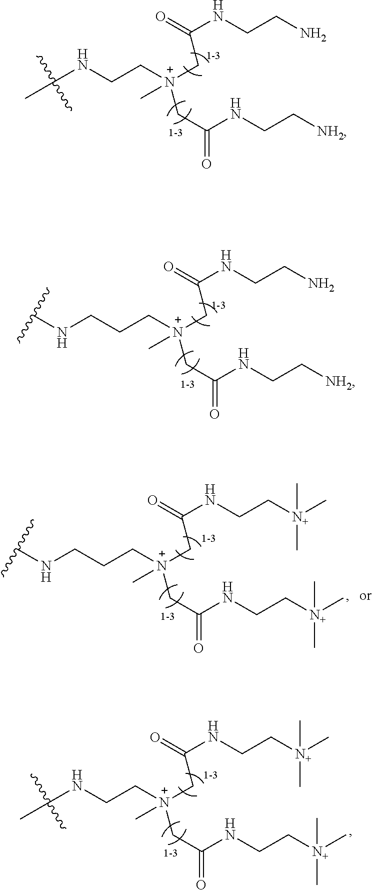

charge balanced with a counterion (e.g., described herein) as necessary. In some embodiments, T^{A1} can be

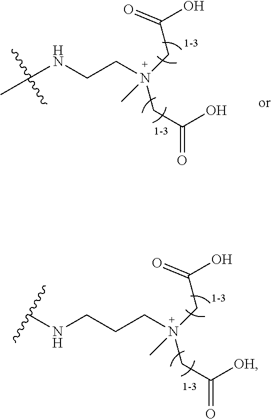

charge balanced with a counterion (e.g., described herein) as necessary. In some embodiments, T^{A1} can be

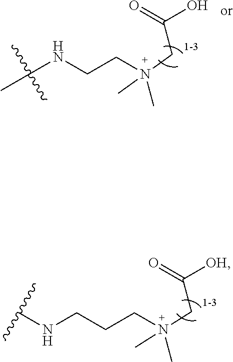

charge balanced with a counterion (e.g., described herein) as necessary. In some embodiments, T^{A1} can be

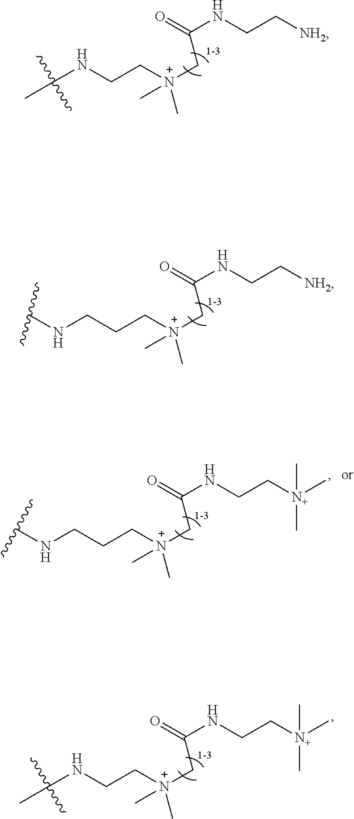

charge balanced with a counterion (e.g., described herein) as necessary. For example, in some embodiments, T^{A1} can be

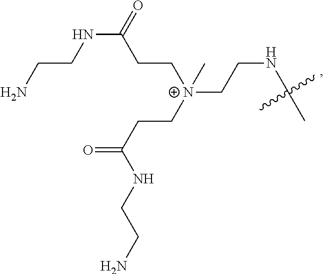 [SMILES: *NCC[N+](C)(CCC(=O)NCCN)CCC(=O)NCCN]

charge balanced with a counterion (e.g., described herein) as necessary. In some embodiments, T^{A1} can be

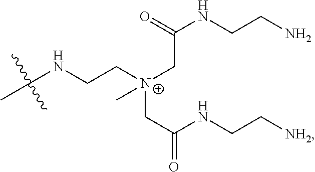 [SMILES: *NCC[N+](C)(CC(=O)NCCN)CC(=O)NCCN]

charge balanced with a counterion (e.g., described herein) as necessary. In some embodiments, T^{A1} can be

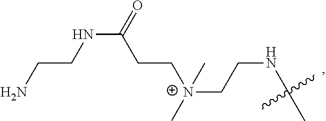 [SMILES: *NCC[N+](C)(C)CCC(=O)NCCN]

charge balanced with a counterion (e.g., described herein) as necessary. In some embodiments, T^{A1} can be

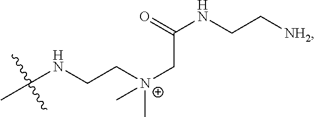 [SMILES: *NCC[N+](C)(C)CC(=O)NCCN]

charge balanced with a counterion (e.g., described herein) as necessary. In some embodiments, T^{A1} can be

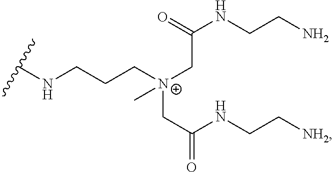 [SMILES: *NCCC[N+](C)(CC(=O)NCCN)CC(=O)NCCN]

charge balanced with a counterion (e.g., described herein) as necessary. In some embodiments, T^{A1} can be

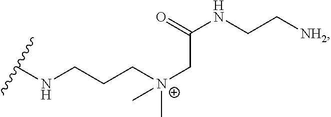 [SMILES: *NCCC[N+](C)(C)CC(=O)NCCN]

charge balanced with a counterion (e.g., described herein) as necessary. In some embodiments, T^{A1} can be

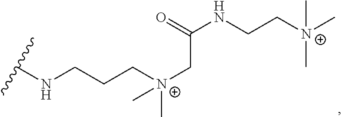 [SMILES: *NCCC[N+](C)(C)CC(=O)NCC[N+](C)(C)C]

charge balanced with a counterion (e.g., described herein) as necessary. In some embodiments, T^{A1} can be

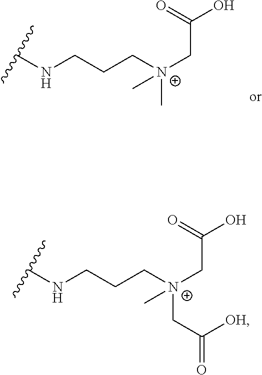 [SMILES: *NCCC[N+](C)(C)CC(=O)O ; *NCCC[N+](C)(CC(=O)O)CC(=O)O]

charge balanced with a counterion (e.g., described herein) as necessary. In some embodiments, T^{A1} can be

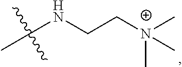

charge balanced with a counterion (e.g., described herein) as necessary. In some embodiments, T^{A1} can be

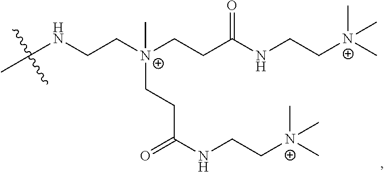 [SMILES: *NCC[N+](C)(CCC(=O)NCC[N+](C)(C)C)CCC(=O)NCC[N+](C)(C)C]

charge balanced with a counterion (e.g., described herein) as necessary. In some embodiments, T^{A1} can be

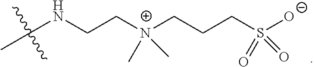 [SMILES: *NCC[N+](C)(C)CCCS(=O)(=O)[O-]]

In some preferred embodiments, T^{A} is T^{A1} or T^{A1}-L^{B}-, wherein T^{A1} is

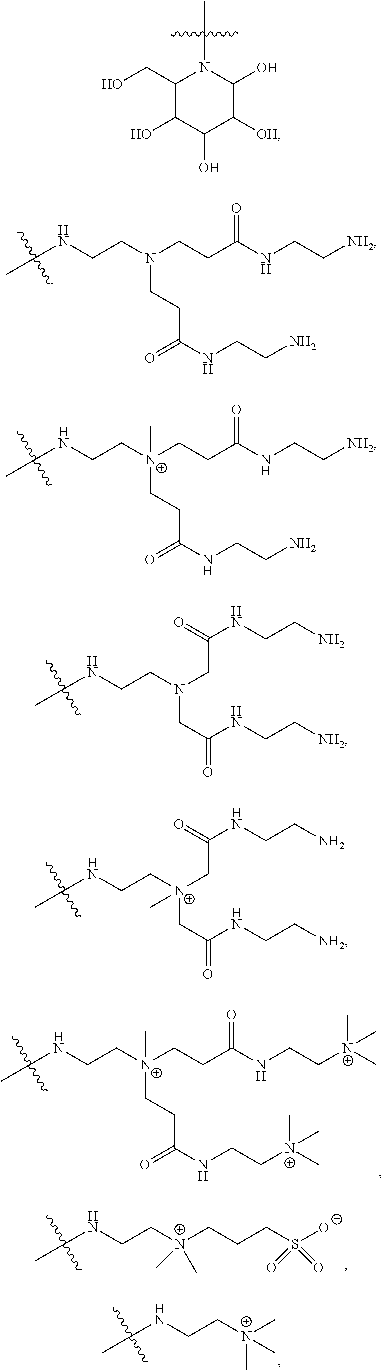

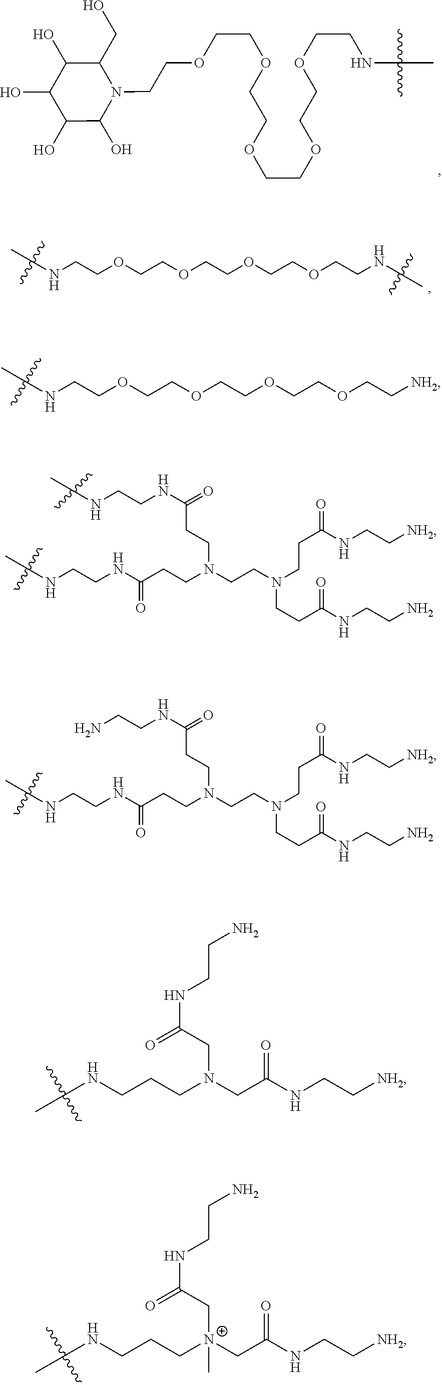

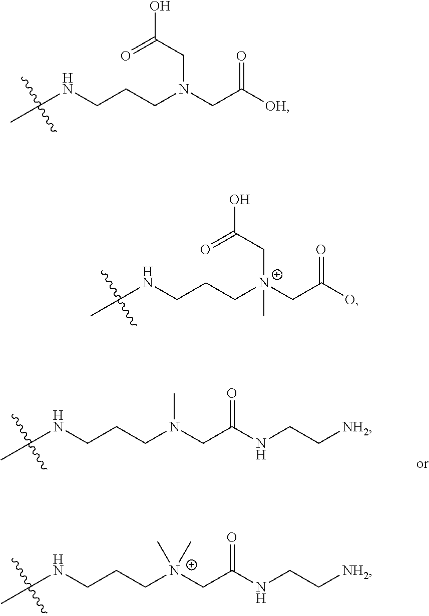

and
wherein L^{B} is —SO₂—, —C(═O)—, or a moiety selected from:

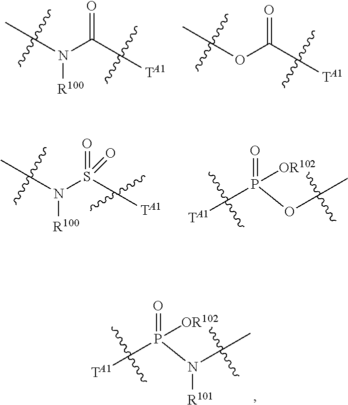

wherein R¹⁰⁰, R¹⁰¹ and R¹⁰² at each occurrence is independently hydrogen, optionally substituted alkyl, or optionally substituted cycloalkyl,
wherein T^{A} is charge balanced with a counterion (e.g., described herein) as necessary.
   Examples of L^{A}-T^{A}

The covalent bond formed between L^{A} and T^{A} is not particularly limited. For example, in some embodiments, the covalent bond(s) between the terminal atom(s) of T^{A} and the end carbon or nitrogen atom(s) of L^{A} is an amide bond. In some embodiments, the covalent bond(s) between the terminal atom(s) of T^{A} and the end atom of L^{A} is a non-amide carbon-nitrogen bond, an ester bond, a non-ester carbon-oxygen bond, a carbon-carbon bond, or a carbon-sulfur bond.

In some preferred embodiments, the compound of Formula III can be characterized as having a Formula III-2, III-3, III-4, III-5, III-6, III-7, III-8, or III-9:

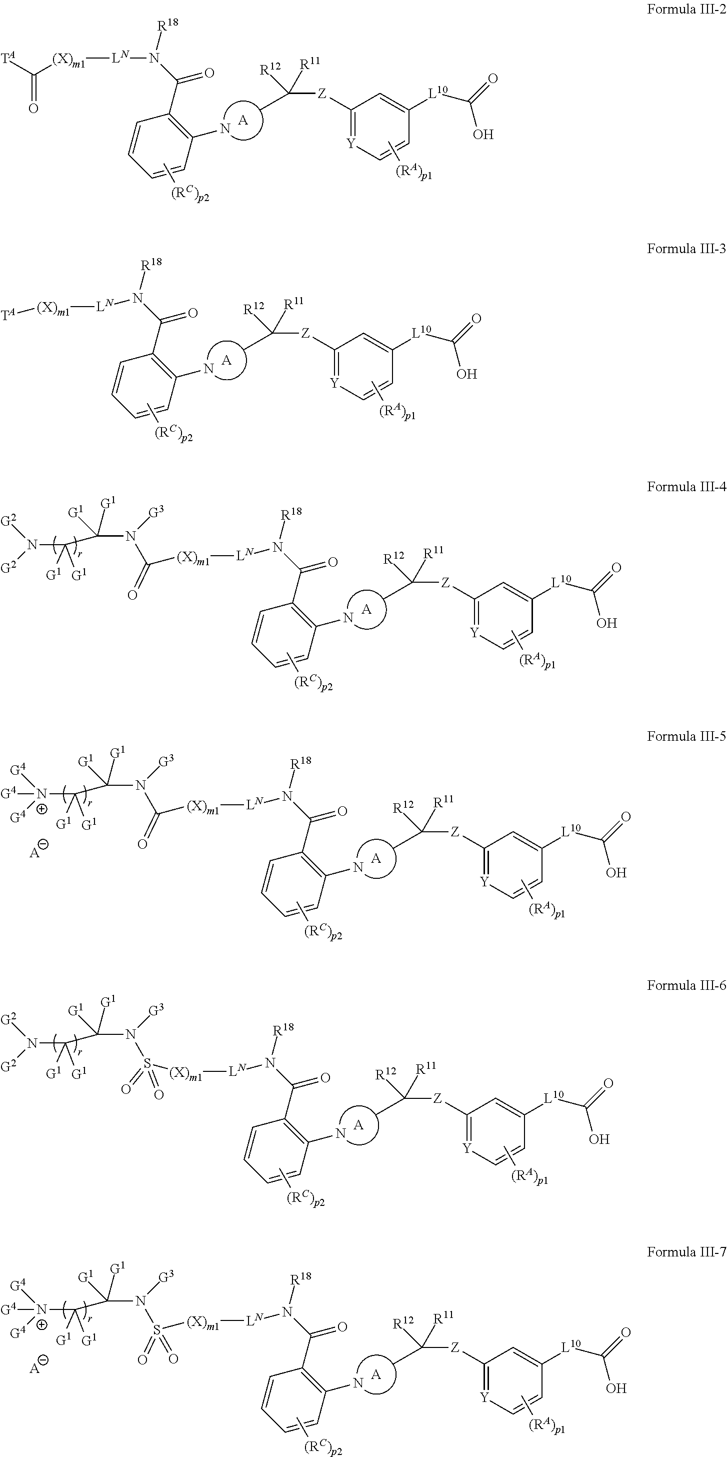

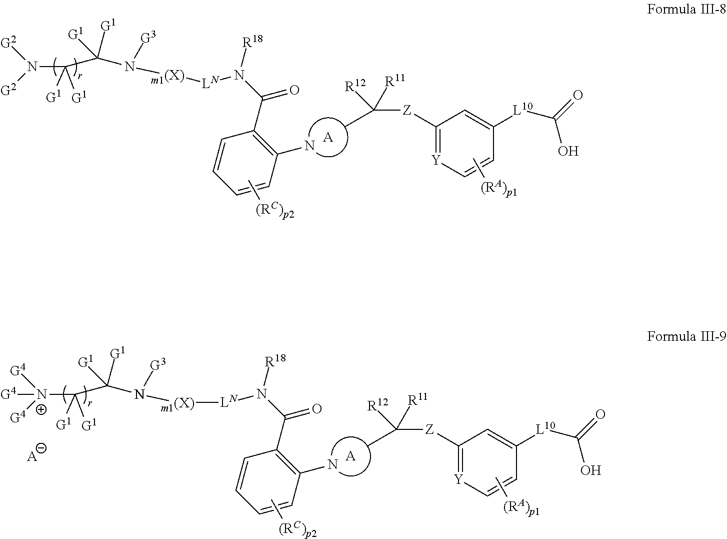

wherein:
X at each occurrence is independently CR₂, C(═O), —C(R)═C(R)—,

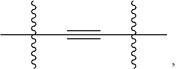 [SMILES: *C#C*]

SiR₂, O, S, SO₂, NR, [NR₂]⁺, or a ring structure, preferably 3-10 membered ring structure, wherein R at each occurrence is independently hydrogen, halogen, optionally substituted C₁₋₄ alkyl, or optionally substituted C₁₋₄ alkoxy, provided that the X group that is directly bonded with L^{N} (when L^{N} is null, it should be understood that the covalent bond is formed with the amide nitrogen atom in Formula III-2 to III-9) is CR₂ or a ring structure, preferably 3-10 membered ring structure;
the integer m1 is at least 12, such as 12-50 (e.g., 12, 14, 16, 18, 20, 22, 24, 30, 40, 50, or any range or value between the recited values),
wherein the hydrophobicity of (X)ₘ₁ is characterized in that (i) when one end X group is C(O) or SO₂, the corresponding compound H—(X)ₘ₁—OH has a cLogP of at least 3, such as between 3-15, preferably at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc., the OH being bonded with C(O) or SO₂; or (ii) the corresponding compound H—(X)ₘ₁—H has a cLogP of at least 3, such as between 3-15, preferably at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc.;
G¹ at each occurrence is independently hydrogen, halogen, optionally substituted C₁₋₄ alkyl, or optionally substituted C₁₋₄ heteroalkyl (such as optionally substituted C₁₋₄ alkoxy), or two gem G¹ group can join to form an ═O, ═NH, or ═N(C₁₋₄ alkyl), or two or more G¹ groups can join to form a ring structure,
the integer r is 1-10, such as 1 or 2,
G² at each occurrence is independently hydrogen, G¹⁰, C(O)-G¹⁰, SO₂G¹⁰, C(O)—NH-G¹⁰, or SO₂NHG¹⁰, C(O)—O-G¹⁰, or SO₂OG¹⁰, wherein G¹⁰ at each occurrence is independently an optionally substituted alkyl, an optionally substituted heteroalkyl, or an optionally substituted 3-10 membered ring structure, which can be carbocyclic, heterocyclic, aromatic, heteroaromatic, or a combination thereof,
G³ is hydrogen or an optionally substituted alkyl (e.g., C₁₋₄ alkyl);
G⁴ at each occurrence is independently hydrogen or G¹⁰, wherein G¹⁰ at each occurrence is independently an optionally substituted alkyl, an optionally substituted heteroalkyl, or an optionally substituted 3-10 membered ring structure, which can be carbocyclic, heterocyclic, aromatic, heteroaromatic, or a combination thereof,
or two G² or one G² and one G¹ or one G¹ and G³ can be joined to form a ring structure such as a 3-10 membered ring structure,
or two G⁴ or one G⁴ and one G¹ or one G¹ and G³ can be joined to form an optionally substituted 3-10 membered ring structure,
A⁻ is a counterion, preferably, a pharmaceutically acceptable anion, as necessary to balance the overall charge of the compound,
wherein the fragment

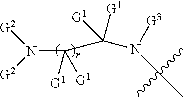

is further characterized in that (i) when the CG¹G¹ group next to the NG³ is not C(═O), the corresponding compound

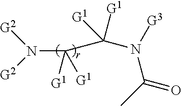

has a cLogP less than 1, preferably, less than 0 (e.g., less than −1, less than −2, less than −3, less than −3.5, less than −4, or even lower), or (ii) when the CG¹G¹ group next to the NG³ is C(═O), the corresponding compound

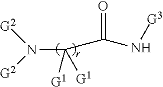

has a cLogP less than 1, preferably, less than 0 (e.g., less than −1, less than −2, less than −3, less than −3.5, less than −4, or even lower),
wherein the fragment

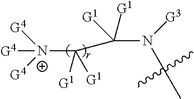

is characterized in that (i) when the CG¹G¹ group next to the NH is not C(═O), the corresponding compound

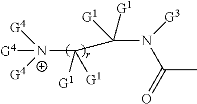

has a cLogP less than 1, preferably, less than 0 (e.g., less than −1, less than −2, less than −3, less than −3.5, less than −4, or even lower), or (ii) when the CG¹G¹ group next to the NH is C(═O), the corresponding compound

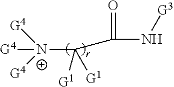

has a cLogP less than 1, preferably, less than 0 (e.g., less than −1, less than −2, less than −3, less than −3.5, less than −4, or even lower), and
wherein the variables T^{A}, L¹⁰, L^{N}, R¹¹, R¹², R¹⁸, R^{A}, R^{C}, ring A, Y, Z, p1, and p2 are defined herein, which include any of those described and preferred herein in any combinations. In some embodiments, L^{N} is null. In some embodiments, L^{N} is null, and two instances of X attached to the amide nitrogen atom in Formula III-2 to III-9 can be a C₁₋₆ alkyl, such as

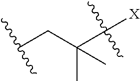

(X is shown to show direction of connection), and the remaining instances of X are as defined herein. In some embodiments, L^{N} is a branched or straight chained C₁₋₆ alkylene, such as

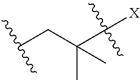

(X is shown to show direction of connection). In some embodiments, G¹ at each occurrence is hydrogen. In some embodiments, one or more instances of G¹ is a C₁₋₄ alkyl such as methyl. In some embodiments, the integer r is 1, 2, 3, 4, or 5. In some embodiments, one or more instances of G² can have a structure of

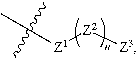

wherein n is an integer of 0-100, preferably, 0-10 (e.g., 0-4, 1-5 or 2-6 etc.), Z¹ is CH₂, C(O), SO, SO₂, CH(C₁₋₄ alkyl), C(C₁₋₄ alkyl)(C₁₋₄ alkyl) or 3-10 membered ring structure, Z² at each occurrence is independently CH₂, O, S, SO, SO₂, C(O), CH(C₁₋₄ alkyl), C(C₁₋₄ alkyl)(C₁₋₄ alkyl), NH, N(C₁₋₄ alkyl), [N(C₁₋₄ alkyl)(C₁₋₄ alkyl)]⁺ or 3-10 membered ring structure, and Z³ is H, C₁₋₄ alkyl, OH, SO₃H, COOH, NH₂, NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, SO₂NH₂, CONH(C₁₋₄ alkyl), CON(C₁₋₄ alkyl)(C₁₋₄ alkyl), SO₂NH(C₁₋₄ alkyl), or SO₂N(C₁₋₄ alkyl)(C₁₋₄ alkyl), wherein each of the C₁₋₄ alkyl is independently selected and optionally substituted, for example, with 1-3 G²⁰ groups defined herein. In some embodiments, none of the Z² is a 3-10 membered ring structure. In some embodiments, one or more instances of Z² is a 3-10 membered ring structure, such as a nitrogen containing ring, for example,

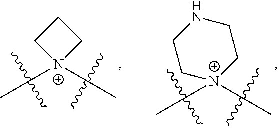 [SMILES: *[N+]1(*)CCC1 ; *[N+]1(*)CCNCC1]

etc. In some embodiments, the Z² directly connects to Z³ is a 3-10 membered ring structure, such as a nitrogen containing ring, for example,

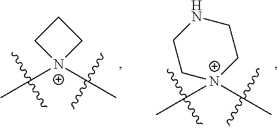 [SMILES: *[N+]1(*)CCC1 ; *[N+]1(*)CCNCC1]

etc. Preferably, at most 2 instances of Z² is a 3-10 membered ring structure. In some embodiments, G² at each occurrence is independently hydrogen, an optionally substituted C₁₋₆ alkyl or an optionally substituted C₁₋₆ heteroalkyl, for example, when substituted, the C₁₋₆ alkyl can be substituted with 1-3 substituents independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), and SO₂N(C₁₋₄ alkyl)(C1. 4 alkyl), wherein each of the C₁₋₄ alkyl is independently selected and optionally substituted, for example, with 1-3 G²⁰ groups defined herein. In some embodiments, G⁴ at each occurrence independently can have a structure of

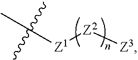

wherein n is an integer of 0-100, preferably, 0-10 (e.g., 0-4, 1-5 or 2-6 etc.), Z¹ is CH₂, C(O), SO₂, CH(C₁₋₄ alkyl), or C (C₁₋₄ alkyl)(C₁₋₄ alkyl), Z² at each occurrence is independently CH₂, O, S, SO, SO₂, C(O), CH(C₁₋₄ alkyl), C(C₁₋₄ alkyl)(C₁₋₄ alkyl), NH, N(C₁₋₄ alkyl), [N(C₁₋₄ alkyl)(C₁₋₄ alkyl)]⁺ or 3-10 membered ring structure, and Z³ is H, C₁₋₄ alkyl, OH, SO₃H, COOH, NH₂, NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, SO₂NH₂, CONH(C₁₋₄ alkyl), CON(C₁₋₄ alkyl)(C₁₋₄ alkyl), SO₂NH(C₁₋₄ alkyl), or SO₂N(C₁₋₄ alkyl)(C₁₋₄ alkyl), wherein each of the C₁₋₄ alkyl is independently selected and optionally substituted, for example, with 1-3 G²⁰ groups defined herein. Preferably, at most 2 instances of Z² is a 3-10 membered ring structure. In some embodiments, none of the Z² is a 3-10 membered ring structure. In some embodiments, one instance of Z² is a 3-10 membered ring structure, such as a nitrogen containing ring, for example,

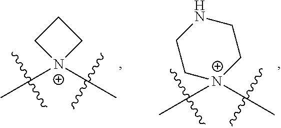 [SMILES: *[N+]1(*)CCC1 ; *[N+]1(*)CCNCC1]

etc. In some embodiments, G⁴ at each occurrence is independently hydrogen, an optionally substituted C₁₋₆ alkyl or an optionally substituted C₁₋₆ heteroalkyl, for example, when substituted, the C₁₋₆ alkyl can be substituted with 1-3 substituents independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), and SO₂N(C₁₋₄ alkyl)(C₁₋₄ alkyl), wherein each of the C₁₋₄ alkyl is independently selected and optionally substituted, for example, with 1-3 G²⁰ groups defined herein. The G²⁰ group herein at each occurrence can be independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), SO₂N(C₁₋₄ alkyl)(C₁₋₄ alkyl), or 3-10 membered ring structure, wherein each of the C₁₋₄ alkyl is independently selected and optionally substituted, for example, with 1-3 G²¹ groups defined herein. The G²¹ group herein at each occurrence can be independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), SO₂N(C₁₋₄ alkyl)(C₁₋₄ alkyl) or 3-10 membered ring structure. In some embodiments, one or more instances of G⁴ is C₁₋₄ alkyl, such as methyl. In some embodiments, one or more instances of G⁴ is C₁₋₄ alkyl, optionally substituted with COOH or an amide derivative, such as CONH—(C₁₋₄ alkylene)-NH₂ or CONH—(C₁₋₄ alkylene)-[N(C₁₋₄ alkyl)(C₁₋₄ alkyl)(C₁₋₄ alkyl)]⁺. For example, in some embodiments, one or two instances of G⁴ is —(C₁₋₄ alkylene)-COOH, such as —CH₂COOH, and any remaining instance(s) of G⁴ is C₁₋₄ alkyl. In some embodiments, one or two instances of G⁴ is —(C₁₋₄ alkylene)-CONH—(C₁₋₄ alkylene)-NH₂, such as

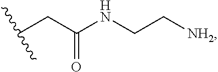 [SMILES: *CC(=O)NCCN]

and any remaining instance(s) of G⁴ is C₁₋₄ alkyl. In some embodiments, one or two instances of G⁴ is —(C₁₋₄ alkylene)-CONH—(C₁₋₄ alkylene)-[N(C₁₋₄ alkyl)(C₁₋₄ alkyl)(C₁₋₄ alkyl)]⁺, such as

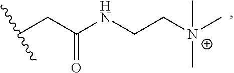 [SMILES: *CC(=O)NCC[N+](C)(C)C]

and any remaining instance(s) of G⁴ is C₁₋₄ alkyl. In preferred embodiments, G³ is hydrogen. In some embodiments, X at each occurrence is independently CH₂, CHCH3, or C(CH₃)₂. In some embodiments, X at each occurrence is CH₂. In some embodiments, up to 6 instances (e.g., 1, 2, 3, 4, 5, or 6) of X is O, and the remaining X is CH₂, CHCH3, or C(CH₃)₂. In some embodiments, up to 10 instances of X is —CH═CH—, and the remaining X is CH₂, CHCH3, or C (CH₃)₂. In some embodiments, the moiety of (X)ₘ₁ has one or more structural features selected from the following: (i) two consecutive X together form-C(O)O— or —C(O) NR—(e.g., C(O)NH or C(O) NCH3); (ii) one or more instances of X can have a ring structure selected from cyclopropane, cyclobutane, bicyclobutane [1.1], cyclopentane, cyclohexane or cycloheptane; and (iii) one or more instances of X can have a ring structure selected from phenyl, or 5- or 6-membered heteroaryl, such as triazole. In some embodiments, within the moiety of (X)ₘ₁, there can be 0, 1, or 2 instances of ring structure, preferably 3-10 membered ring structure. In some embodiments, within the moiety of (X)ₘ₁, there is one triazole ring. For example, in some embodiments, the moiety of (X)ₘ₁ can have a structure according to

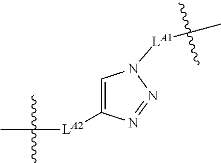

as defined and preferred herein. For example, in some embodiments, LAI and L^{A2} are each independently a bond, —C₁₋₃₀ alkylene-, or —C₁₋₃₀ heteroalkylene-containing 1-6 heteroatoms independently selected from O, N, and S, wherein the sulfur atom(s), if present, is optionally oxidized, wherein the total number of non-hydrogen atoms of (X)ₘ₁ is between 15-50, such as 18, 20, 25, 30, 35, 40, 45, or 50, or any ranges between the recited value. In some embodiments, L^{A1} is a bond. In some embodiments, L^{A1} is a C₁₋₂₀ alkylene, such as a linear alkylene, (CH₂)₁₋₂₀. In some embodiments, L^{A1} is a C₁₋₁₀ heteroalkylene having 1-3 oxygen atoms. In some embodiments, L^{A2} is a bond. In some embodiments, L^{A2} is a C₁₋₂₀ alkylene, such as a linear alkylene, (CH₂)₁₋₂₀. In some embodiments, L^{A2} is a C₁₋₁₀ heteroalkylene having 1-3 oxygen atoms, such as —O—(C₁₋₁₀ alkylene)-, etc. Either of L^{A1} and L^{A2} can be attached to L^{N}(when L^{N} is null, it should be understood that the covalent bond is formed with the amide nitrogen atom in Formula III-2 to III-9 above).

### Residue of GPR40 Agonist

The variables L¹⁰, R¹¹, R¹², R¹⁸, R^{A}, R^{C}, ring A, ring B, Y, Z, p1, and p2 in Formula III or III-B are not particularly limited. However, in preferred embodiments, the variables in Formula III or III-B are such that at least one corresponding compound according to Formula GPR-3,

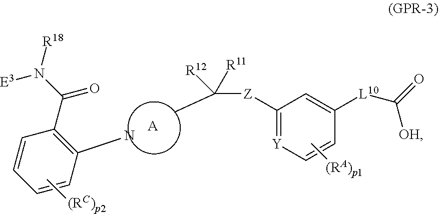

### or Formula GPR-3B,

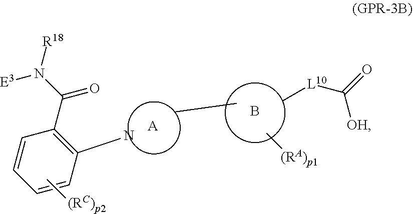

wherein E³ is E^{3A} or

L^{N}-E^{3A}, wherein E^{3A} is hydrogen, N3,

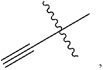 [SMILES: *C#C]

C₁₋₄ alkyl,

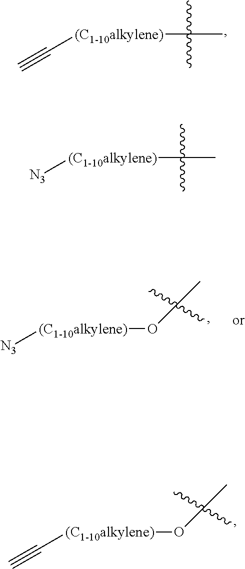

and L^{N} is defined herein (such as null or a C₁₋₆ alkyl alkylene), is a GPR40 agonist, preferably, having an EC50 of less than 100 nM as measured according to Biological Example 1 herein.

Typically, p1 in Formula III or III-B is 0.

In some embodiments, p1 in Formula III or III-B is 1.

Typically, R^{A} at each occurrence is independently F, Cl, CN, C₁₋₄ alkyl optionally substituted with 1-3 fluorine, or C₁₋₄ alkoxy optionally substituted with 1-3 fluorine.

Typically, p2 in Formula III or III-B is 0.

In some embodiments, p2 in Formula III or III-B is 1, and R^{C} is F, Cl, CN, C₁₋₄ alkyl optionally substituted with 1-3 fluorine, or C₁₋₄ alkoxy optionally substituted with 1-3 fluorine.

In Formula III, Y is typically N.

Preferably, Z in Formula III is O.

In some embodiments, R¹¹ and R¹² are both hydrogen.

In some preferred embodiments, L¹⁰ is characterized as having a structure of

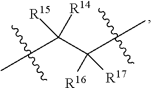

wherein CR₁₆R¹⁷ is bonded to the COOH group, and wherein:
R¹⁴ is hydrogen, C₁₋₆ alkyl, C₂₋₆ alkenyl, C₂₋₆ alkynyl, C₃₋₆ cycloalkyl, phenyl, 5 or 6 membered heteroaryl, or 3-7 membered heterocyclyl, wherein each of the alkyl, alkenyl, alkynyl, cycloalkyl, phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy, and R¹⁵, R¹⁶ and R¹⁷ are each independently hydrogen or C₁₋₄ alkyl; or
R¹⁴ and R¹⁵ are joined to form a 3-7 membered ring with 0, 1, or 2 heteroatoms selected from O, N, or S. In some embodiments, R¹⁶ and R¹⁷ are both hydrogen, or one of R¹⁶ and R¹⁷ is hydrogen and the other of R¹⁶ and R¹⁷ is methyl. In some embodiments, one of R¹⁴ and R¹⁵ is hydrogen, and the other of R¹⁴ and R¹⁵ is C₁₋₆ alkyl, C₂₋₆ alkenyl, C₂₋₆ alkynyl, or C₃₋₆ cycloalkyl. In some embodiments, R¹⁴ and R¹⁵ are joined to form a C₃₋₆ cycloalkyl.

For example, in some embodiments, L¹⁰ is

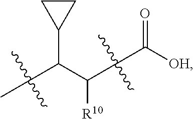

wherein R¹⁰ is hydrogen or C₁₋₄ alkyl, such as methyl.
R¹⁸ in Formula III or III-B is typically an optionally substituted phenyl or an optionally substituted 5 or 6 membered heteroaryl having 1-4 ring heteroatoms.

In some embodiments, R¹⁸ is an optionally substituted 6-membered heteroaryl ring. In some embodiments, R¹⁸ is a 6-membered heteroaryl ring, such as a pyridyl ring,

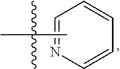

which is optionally substituted with 1-3 substituents independently selected from F, CI, CN, OH, C₁₋₆ alkyl, C₁₋₆ alkyl heteroalkyl, C₃₋₆ cycloalkyl, C₁₋₆ alkyl alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), and C₁₋₄ alkyl optionally substituted with F. In some embodiments, R¹⁸ is

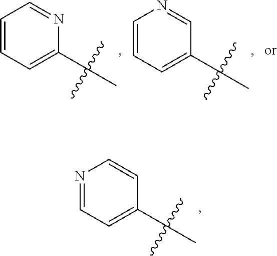 [SMILES: *c1ccccn1 ; *c1cccnc1 ; *c1ccncc1]

each of which is optionally substituted with 1-3 substituents independently selected from F, Cl, CN, OH, C₁₋₆ alkyl, C₁₋₆ alkyl heteroalkyl, C₃₋₆ cycloalkyl, C₁₋₆ alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), and C₁₋₄ alkyl optionally substituted with F.

Ring A for Formula III or III-B is a nitrogen containing heterocyclic structure, with at least one nitrogen that is bonded with the phenyl ring shown in Formula III or III-B.

In some embodiments, Ring A for Formula III or III-B is a 4-8 membered optionally substituted monocyclic saturated heterocyclic ring having one or two ring heteroatoms independently selected from S, O, and N, provided at least one of the ring heteroatom is nitrogen. For example, in some embodiments, Ring A is selected from:

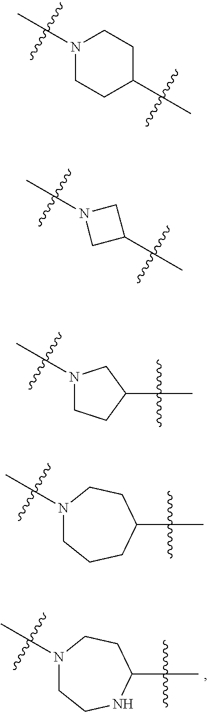 [SMILES: *C1CCN(*)CC1 ; *C1CN(*)C1 ; *C1CCN(*)C1 ; *C1CCCN(*)CC1 ; *C1CCN(*)CCN1]

each of which is optionally substituted with 1-2 substituents independently selected from F, OH, NH₂, NH(C₁₋₄ alkyl), N(C₁₋₄ alkyl)(C₁₋₄ alkyl), C₁₋₄ alkyl optionally substituted with 1-3 fluorine, or C₁₋₄ alkoxy optionally substituted with 1-3 fluorine.

In some embodiments, Ring A for Formula III or III-B can also be a bicyclic or polycyclic 6-12 membered optionally substituted saturated heterocyclic ring having one or two ring heteroatoms independently selected from S, O, and N, provided at least one of the ring heteroatom is nitrogen.

In some preferred embodiments, the compound of Formula III or III-B can be characterized as having a Formula III-1-A or III-B-2, respectively:

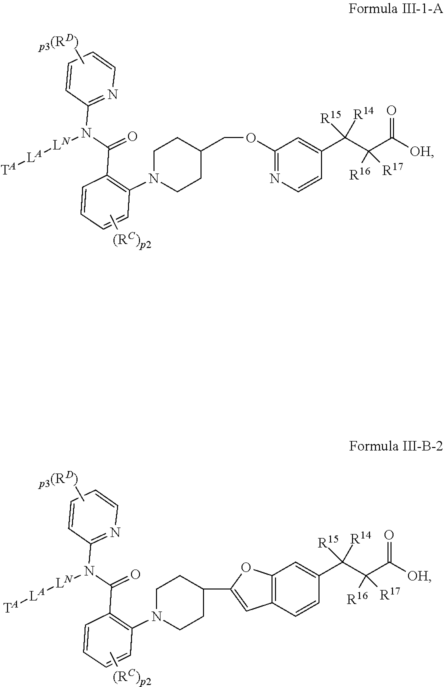

wherein:
R¹⁴ is hydrogen, C₁₋₆ alkyl, C₂₋₆ alkenyl, C₂₋₆ alkynyl, C₃₋₆ cycloalkyl, phenyl, 5 or 6 membered heteroaryl, or 3-7 membered heterocyclyl, wherein each of the alkyl, alkenyl, alkynyl, cycloalkyl, phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy, and R¹⁵, R¹⁶ and R¹⁷ are each independently hydrogen or C₁₋₄ alkyl; or
R¹⁴ and R¹⁵ are joined to form a 3-7 membered ring with 0, 1, or 2 heteroatoms selected from O, N, or S;
R^{D} at each occurrence is independently F, Cl, C₁₋₄ alkyl optionally substituted with 1-3 F, or C₁₋₄ alkoxy optionally substituted with 1-3 F, and
wherein p3 is 0, 1, 2, or 3,
and the variables T^{A}, L^{N}, and L^{A} include any of those described and preferred herein in any combinations. For example, in some embodiments, L^{N} is null. In some embodiments, L^{N} is a branched or straight chained C₁₋₆ alkyl alkylene, such as

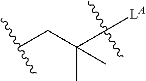

(L^{A} is shown to show direction of connection). In some embodiments, R¹⁶ and R¹⁷ are both hydrogen. In some embodiments, one of R¹⁶ and R¹⁷ is hydrogen and the other of R¹⁶ and R¹⁷ is methyl. In some embodiments, one of R¹⁴ and R¹⁵ is hydrogen, and the other of R¹⁴ and R¹⁵ is C₁₋₆ alkyl, C₂₋₆ alkenyl, C₂₋₆ alkynyl, or C₃₋₆ cycloalkyl. In some embodiments, R¹⁴ and R¹⁵ are joined to form a C₃₋₆ cycloalkyl.

In some preferred embodiments, the compound of Formula III or III-B can be characterized as having a Formula III-1-A-1 or III-B-3, respectively:

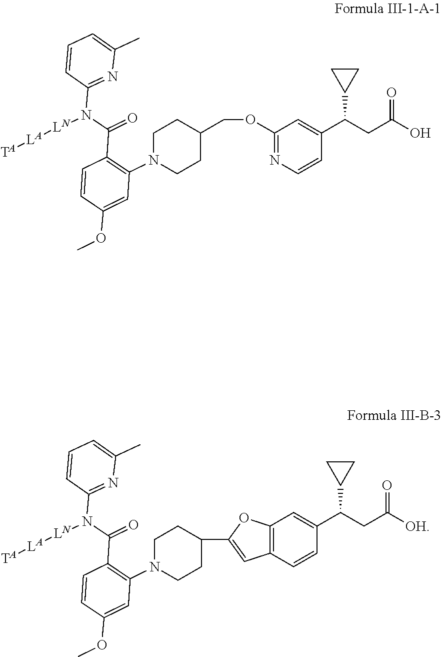

wherein the variables T^{A}, L^{N}, and L^{A} include any of those described and preferred herein in any combinations. For example, in some embodiments, L^{N} is null. In some embodiments, L^{N} is a branched or straight chained C₁₋₆ alkyl alkylene, such as

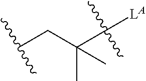

(L^{A} is shown to show direction of connection).

In preferred embodiments, L^{A} in Formula III-1-A (e.g., III-1-A-1), III-B-2, or III-B-3 can be —X₁₂₋₃₀—C(O)—, wherein the C(O) is directly bonded with T^{A}, and wherein X at each occurrence is independently CR₂, —C(R)═C(R)—,

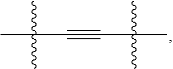 [SMILES: *C#C*]

SiR₂, C(O), O, NR, S, SO₂, or a ring structure, preferably 3-10 membered ring structure, provided that the end X group (i.e., the X group that is directly bonded with L^{N}, when L^{N} is null, it should be understood that the X is directly bonded with the amide nitrogen atom in Formula III or III-B, is CR₂ or a ring structure, preferably 3-10 membered ring structure, wherein R at each occurrence is independently hydrogen, halogen, optionally substituted C₁₋₄ alkyl, or optionally substituted C₁₋₄ alkoxy, typically hydrogen or C₁₋₄ alkyl, wherein the longest chain length of L^{A} is at least that of —(CH₂)₁₂—, such as at least that of —(CH₂)₁₄—, at least that of —(CH₂)₁₆—, at least that of —(CH₂)₁₈—, and wherein the hydrophobicity of L^{A} can be characterized in that the corresponding compound H-X12-30—C(O)—OH has a cLogP of at least 3, such as between 3-15, preferably, at least 4 (e.g., at least 4.5, at least 5, at least 5.5, at least 6, at least 6.5, at least 7, etc.), such as has a cLogP of 3-10, 4-12, 4.5-9, 5-11, 5.5-10.5, 6-14, 6.5-13, etc. Preferably, the end X group is CR₂ such as CH₂. Preferably, the total number of non-hydrogen atoms of L^{A} is between 15-50, such as 18, 20, 25, 30, 35, 40, 45, or 50, or any ranges between the recited value. In some preferred embodiments, none of the X groups is a ring structure. In some preferred embodiments, one instance of X is a ring structure, preferably 3-10 membered ring structure. For example, in some embodiments, one instance of X is triazole and the remaining X groups are each independently CR₂, —C(R)═C(R)—,

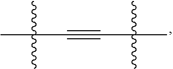 [SMILES: *C#C*]

or O, wherein R at each occurrence is independently hydrogen or C₁₋₄ alkyl. In some embodiments, none of the X groups is a ring structure, and each X is independently CR₂, —C(R)═C(R)—,

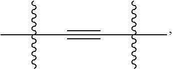 [SMILES: *C#C*]

or O, wherein R at each occurrence is independently hydrogen or C₁₋₄ alkyl (preferably, methyl). In some embodiments, all instances of X is CR₂, wherein R at each occurrence is independently hydrogen or C₁₋₄ alkyl (preferably, methyl). In some embodiments, up to 6 instances (e.g., 1, 2, 3, 4, 5, or 6) of X can be O. In some embodiments, one or two instances of X is O, and all other instances of X is CR₂, wherein R at each occurrence is independently hydrogen or C₁₋₄ alkyl (preferably, methyl). In some embodiments, one or two instances of X is O, one X is triazole, and all other instances of X is CR₂, wherein R at each occurrence is independently hydrogen or C₁₋₄ alkyl (preferably, methyl). In some embodiments, R is hydrogen.

In some preferred embodiments, T^{A} in Formula III-1-A (e.g., III-1-A-1), III-B-2, or III-B-3 can be

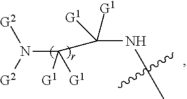

wherein G¹ at each occurrence is independently hydrogen, halogen, optionally substituted C₁₋₄ alkyl, or optionally substituted C₁₋₄ heteroalkyl (such as optionally substituted C₁₋₄ alkoxy), or two gem G¹ group can join to form an ═O, ═NH, or ═N(C₁₋₄ alkyl), or two or more G¹ groups can join to form a ring structure,
the integer r is 1-10, such as 1 or 2,
G² at each occurrence is independently hydrogen, G¹⁰, C(O)-G¹⁰, SO₂G¹⁰, C(O)—NH-G¹⁰, or SO₂NHG¹⁰, C(O)—O-G¹⁰, or SO₂OG¹⁰, wherein G¹⁰ at each occurrence is independently an optionally substituted C₁₋₆ alkyl, an optionally substituted C₁₋₆ heteroalkyl, or an optionally substituted 3-10 membered ring structure, which can be carbocyclic, heterocyclic, aromatic, heteroaromatic, or a combination thereof, wherein the fragment is further characterized in that (i) when the CG¹G¹ group next to the NH is not C(═O), the corresponding compound

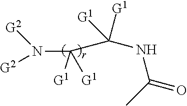

has a cLogP less than 1, preferably, less than 0 (e.g., less than −1, less than −2, less than −3, less than −3.5, less than −4, or even lower), or (ii) when the CG¹G¹ group next to the NH is C(═O), the corresponding compound

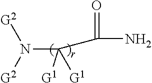

has a cLogP less than 1, preferably, less than 0 (e.g., less than −1, less than −2, less than −3, less than −3.5, less than −4, or even lower).

In some preferred embodiments, T^{A} in Formula III-1-A (e.g., III-1-A-1), III-B-2, or III-B-3 can be characterized as having a structure of:

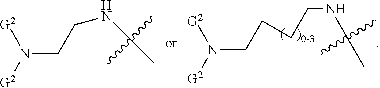

In some embodiments, one or both instances of G² can have a structure of

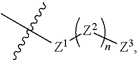

wherein n is an integer of 0-100, preferably, 0-10 (e.g., 0-4, 1-5 or 2-6 etc.), Z¹ is CH₂, C(O), SO, SO₂, CH(C₁₋₄ alkyl), C(C₁₋₄ alkyl)(C₁₋₄ alkyl) or 3-10 membered ring structure, Z² at each occurrence is independently CH₂, O, S, SO, SO₂, C(O), CH(C₁₋₄ alkyl), C(C₁₋₄ alkyl)(C₁₋₄ alkyl), NH, N(C₁₋₄ alkyl), [N(C₁₋₄ alkyl)(C₁₋₄ alkyl)]⁺ or 3-10 membered ring structure, and Z³ is H, C₁₋₄ alkyl, OH, SO₃H, COOH, NH₂, NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, SO₂NH₂, CONH(C₁₋₄ alkyl), CON(C₁₋₄ alkyl)(C₁₋₄ alkyl), SO₂NH(C₁₋₄ alkyl), or SO₂N(C₁₋₄ alkyl)(C₁₋₄ alkyl), wherein each of the C₁₋₄ alkyl is independently selected and optionally substituted, for example, with 1-3 G²⁰ groups defined herein. In some embodiments, none of the Z² is a 3-10 membered ring structure. In some embodiments, one or more instances of Z² is a 3-10 membered ring structure, such as a nitrogen containing ring, for example,

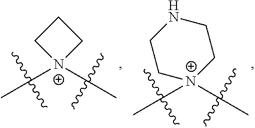 [SMILES: *[N+]1(*)CCC1 ; *[N+]1(*)CCNCC1]

etc. In some embodiments, the Z² directly connects to Z³ is a 3-10 membered ring structure, such as a nitrogen containing ring, for example,

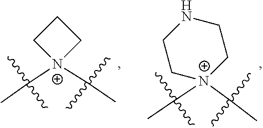 [SMILES: *[N+]1(*)CCC1 ; *[N+]1(*)CCNCC1]

etc. Preferably, at most 2 instances of Z² is a 3-10 membered ring structure. In some embodiments, G² at each occurrence is independently hydrogen, an optionally substituted C₁₋₆ alkyl or an optionally substituted C₁₋₆ heteroalkyl, for example, when substituted, the C₁₋₆ alkyl can be substituted with 1-3 substituents independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), and SO₂N(C₁₋₄ alkyl)(C1-4 alkyl), wherein each of the C₁₋₄ alkyl is independently selected and optionally substituted, for example, with 1-3 G²⁰ groups defined herein. The G²⁰ group herein at each occurrence can be independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), SO₂N(C₁₋₄ alkyl)(C₁₋₄ alkyl), or 3-10 membered ring structure, wherein each of the C₁₋₄ alkyl is independently selected and optionally substituted, for example, with 1-3 G²¹ groups defined herein. The G²¹ group herein at each occurrence can be independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), SO₂N(C₁₋₄ alkyl)(C₁₋₄ alkyl) or 3-10 membered ring structure. In some embodiments, one or more instances of G² is C₁₋₄ alkyl, optionally substituted with COOH or an amide derivative, such as CONH—(C₁₋₄ alkylene)-NH₂ or CONH—(C₁₋₄ alkylene)-[N(C₁₋₄ alkyl)(C₁₋₄ alkyl)(C₁₋₄ alkyl)]⁺. For example, in some embodiments, one or two instances of G² is —(C₁₋₄ alkylene)-COOH, such as —CH₂COOH, and any remaining instance(s) of G² is C₁₋₄ alkyl. In some embodiments, one or two instances of G² is —(C₁₋₄ alkylene)-CONH—(C₁₋₄ alkylene)-NH₂, such as

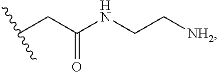 [SMILES: *CC(=O)NCCN]

and any remaining instance(s) of G² is C₁₋₄ alkyl. In some embodiments, one or two instances of G² is —(C₁₋₄ alkylene)-CONH—(C₁₋₄ alkylene)-[N(C₁₋₄ alkyl)(C₁₋₄ alkyl)(C₁₋₄ alkyl)]⁺, such as

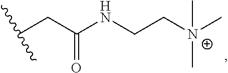 [SMILES: *CC(=O)NCC[N+](C)(C)C]

and any remaining instance(s) of G² is C₁₋₄ alkyl. In some embodiments, T^{A} can be

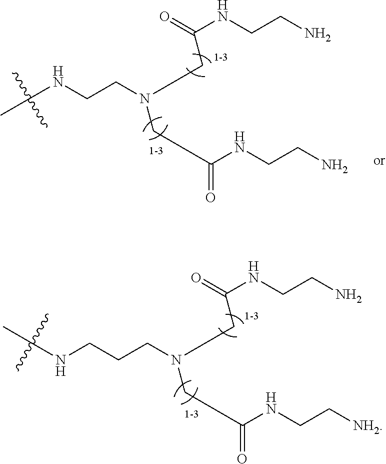

In some embodiments, T^{A} can be

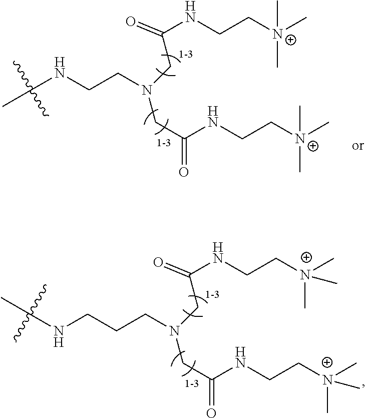

charge balanced with a counterion (e.g., described herein) as necessary. In some embodiments, T^{A} can be

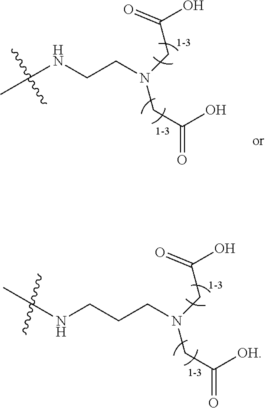

In some embodiments, T^{A} can be

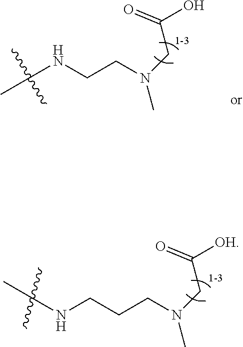

In some embodiments, T^{A} can be

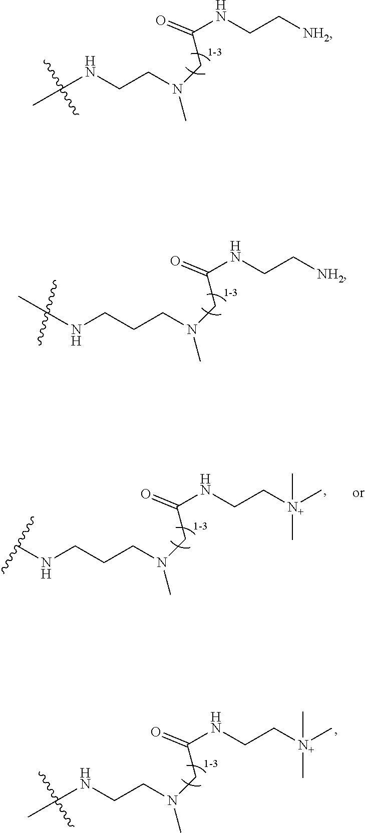

charge balanced with a counterion (e.g., described herein) as necessary. In some embodiments, T^{A} can be

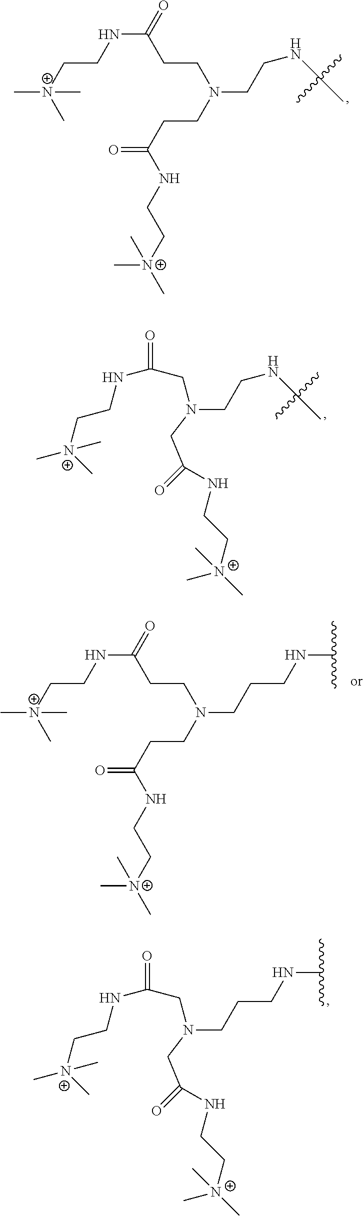 [SMILES: *NCCN(CCC(=O)NCC[N+](C)(C)C)CCC(=O)NCC[N+](C)(C)C ; *NCCN(CC(=O)NCC[N+](C)(C)C)CC(=O)NCC[N+](C)(C)C ; *NCCCN(CCC(=O)NCC[N+](C)(C)C)CCC(=O)NCC[N+](C)(C)C ; *NCCCN(CC(=O)NCC[N+](C)(C)C)CC(=O)NCC[N+](C)(C)C]

charge balanced with a counterion (e.g., described herein) as necessary. For example, in some embodiments, T^{A} can be

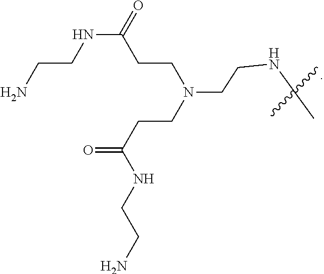 [SMILES: *NCCN(CCC(=O)NCCN)CCC(=O)NCCN]

In some embodiments, T^{A} can be

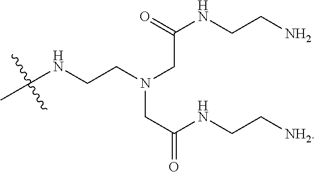 [SMILES: *NCCN(CC(=O)NCCN)CC(=O)NCCN]

In some embodiments, T^{A} can be

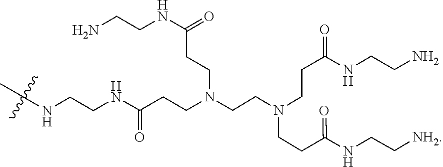 [SMILES: *NCCNC(=O)CCN(CCC(=O)NCCN)CCN(CCC(=O)NCCN)CCC(=O)NCCN]

In some embodiments, T^{A} can be

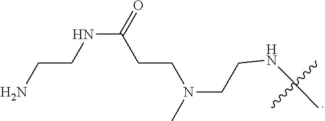 [SMILES: *NCCN(C)CCC(=O)NCCN]

In some embodiments, T^{A} can be

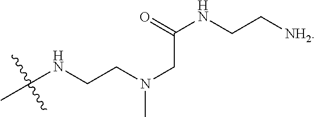 [SMILES: *NCCN(C)CC(=O)NCCN]

In some embodiments, T^{A} can be

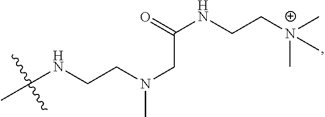 [SMILES: *NCCN(C)CC(=O)NCC[N+](C)(C)C]

charge balanced with a counterion (e.g., described herein) as necessary. In some embodiments, T^{A} can be

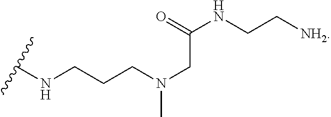 [SMILES: *NCCCN(C)CC(=O)NCCN]

In some embodiments, T^{A} can be

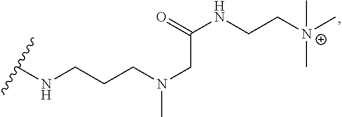 [SMILES: *NCCCN(C)CC(=O)NCC[N+](C)(C)C]

charge balanced with a counterion (e.g., described herein) as necessary. In some embodiments, T^{A} can be

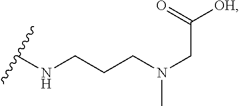 [SMILES: *NCCCN(C)CC(=O)O]

In some preferred embodiments, T^{A} in Formula III-1-A (e.g., III-1-A-1), III-B-2, or III-B-3 can be characterized as having a structure of:

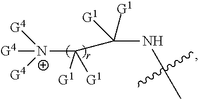

wherein G¹ at each occurrence is independently hydrogen, halogen, optionally substituted C₁₋₄ alkyl, or optionally substituted C₁₋₄ heteroalkyl (such as optionally substituted C₁₋₄ alkoxy), or two gem G¹ group can join to form an ═O, ═NH, or ═N(C₁₋₄ alkyl), or two or more G¹ groups can join to form a ring structure,
the integer r is 1-10, such as 1 or 2,
G⁴ at each occurrence is independently hydrogen or G¹⁰, wherein G¹⁰ at each occurrence is independently an optionally substituted C₁₋₆ alkyl, an optionally substituted C₁₋₆ heteroalkyl, or an optionally substituted 3-10 membered ring structure, which can be carbocyclic, heterocyclic, aromatic, heteroaromatic, or a combination thereof, wherein the fragment

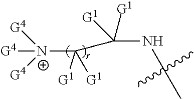

is characterized in that (i) when the CG¹G¹ group next to the NH is not C(═O), the corresponding compound

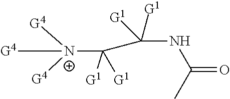

has a cLogP less than 1, preferably, less than 0 (e.g., less than −1, less than −2, less than −3, less than −3.5, less than −4, or even lower), or (ii) when the CG¹G¹ group next to the NH is C(═O), the corresponding compound

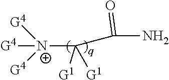

has a cLogP less than 1, preferably, less than 0 (e.g., less than −1, less than −2, less than −3, less than −3.5, less than −4, or even lower).

In some preferred embodiments, T^{A} in Formula III-1-A (e.g., III-1-A-1), III-B-2, or III-B-3 can be characterized as having a structure of:

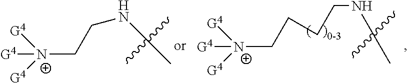

In some embodiments, one or more instances of G⁴ can have a structure of

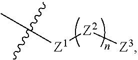

n is an integer of 0-100, preferably, 0-10 (e.g., 0-4, 1-5 or 2-6 etc.), Z¹ is CH₂, C(O), SO₂, CH(C₁₋₄ alkyl), or C(C₁₋₄ alkyl)(C₁₋₄ alkyl), Z² at each occurrence is independently CH₂, O, S, SO, SO₂, C(O), CH(C₁₋₄ alkyl), C(C₁₋₄ alkyl)(C₁₋₄ alkyl), NH, N(C₁₋₄ alkyl), [N(C₁₋₄ alkyl)(C₁₋₄ alkyl)]⁺ or 3-10 membered ring structure, and Z³ is H, C₁₋₄ alkyl, OH, SO₃H, COOH, NH₂, NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, SO₂NH₂, CONH(C₁₋₄ alkyl), CON(C₁₋₄ alkyl)(C₁₋₄ alkyl), SO₂NH(C₁₋₄ alkyl), or SO₂N(C₁₋₄ alkyl)(C₁₋₄ alkyl), wherein each of the C₁₋₄ alkyl is independently selected and optionally substituted, for example, with 1-3 G²⁰ groups defined herein. Preferably, at most 2 instances of Z² is a 3-10 membered ring structure. In some embodiments, none of the Z² is a 3-10 membered ring structure. In some embodiments, one instance of Z² is a 3-10 membered ring structure, such as a nitrogen containing ring, for example,

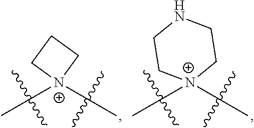 [SMILES: *[N+]1(*)CCC1 ; *[N+]1(*)CCNCC1]

etc. In some embodiments, G⁴ at each occurrence is independently hydrogen, an optionally substituted C₁₋₆ alkyl or an optionally substituted C₁₋₆ heteroalkyl, for example, when substituted, the C₁₋₆ alkyl can be substituted with 1-3 substituents independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), and SO₂N(C₁₋₄ alkyl)(C1. 4 alkyl), wherein each of the C₁₋₄ alkyl is independently selected and optionally substituted, for example, with 1-3 G²⁰ groups defined herein. The G²⁰ group herein at each occurrence can be independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), SO₂N(C₁₋₄ alkyl)(C₁₋₄ alkyl), or 3-10 membered ring structure, wherein each of the C₁₋₄ alkyl is independently selected and optionally substituted, for example, with 1-3 G²¹ groups defined herein. The G²¹ group herein at each occurrence can be independently selected from OH, NH₂, oxo (═O), NH(C₁₋₄ alkyl), N(C₁₋₄ alkyl)(C₁₋₄ alkyl), [N(C₁₋₄ alkyl)(C₁₋₄ alkyl)(C₁₋₄ alkyl)]⁺, C(O)NH₂, C(O)OH, SO₂NH₂, SO₃H, CONH(C₁₋₄ alkyl), CON(C₁₋₄ alkyl)(C₁₋₄ alkyl), SO₂NH(C₁₋₄ alkyl), SO₂N(C₁₋₄ alkyl)(C₁₋₄ alkyl) or 3-10 membered ring structure. In some embodiments, one or more instances of G⁴ is C₁₋₄ alkyl, such as methyl. In some embodiments, one or more instances of G⁴ is C₁₋₄ alkyl, optionally substituted with COOH or an amide derivative, such as CONH—(C₁₋₄ alkylene)-NH₂ or CONH—(C₁₋₄ alkylene)-[N(C₁₋₄ alkyl)(C₁₋₄ alkyl)(C₁₋₄ alkyl)]⁺. For example, in some embodiments, one or two instances of G⁴ is —(C₁₋₄ alkylene)-COOH, such as —CH₂COOH, and any remaining instance(s) of G⁴ is C14 alkyl. In some embodiments, one or two instances of G⁴ is —(C₁₋₄ alkylene)-CONH—(C₁₋₄ alkylene)-NH₂, such as

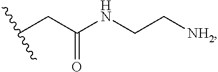 [SMILES: *CC(=O)NCCN]

and any remaining instance(s) of G⁴ is C₁₋₄ alkyl. In some embodiments, one or two instances of G⁴ is —(C₁₋₄ alkylene)-CONH—(C₁₋₄ alkylene)-[N(C₁₋₄ alkyl)(C₁₋₄ alkyl)(C₁₋₄ alkyl)]⁺, such as

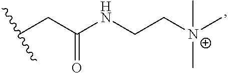 [SMILES: *CC(=O)NCC[N+](C)(C)C]

and any remaining instance(s) of G⁴ is C₁₋₄ alkyl. In some embodiments, T^{A} can be

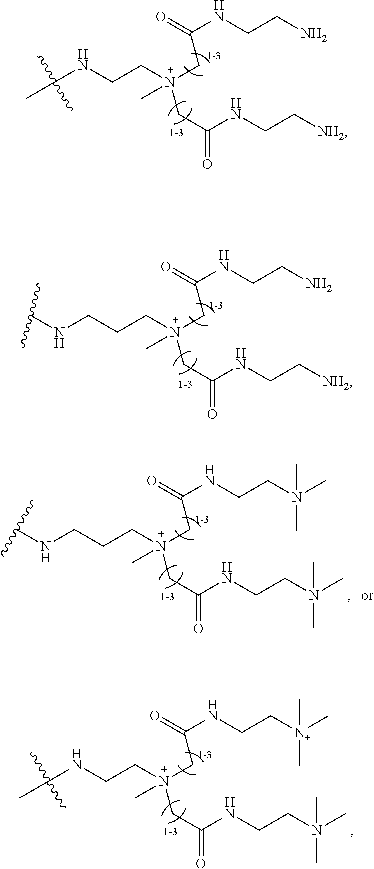

charge balanced with a counterion (e.g., described herein) as necessary. In some embodiments, T^{A} can be

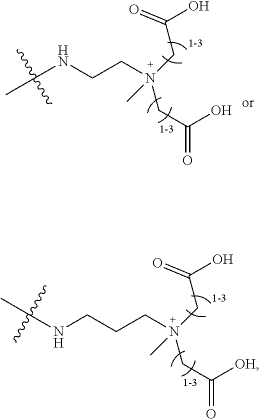

charge balanced with a counterion (e.g., described herein) as necessary. In some embodiments, T^{A} can be

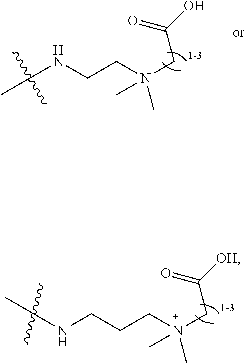

charge balanced with a counterion (e.g., described herein) as necessary. In some embodiments, T^{A} can be

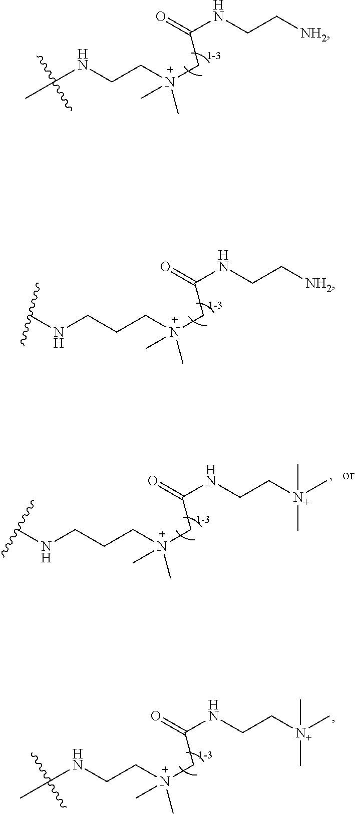

charge balanced with a counterion (e.g., described herein) as necessary. For example, in some embodiments, T^{A} can be

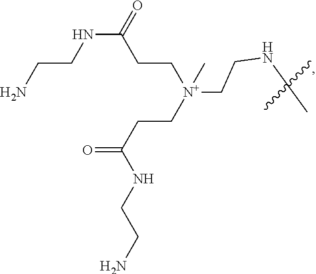 [SMILES: *NCC[N+](C)(CCC(=O)NCCN)CCC(=O)NCCN]

charge balanced with a counterion (e.g., described herein) as necessary. In some embodiments, T^{A} can be

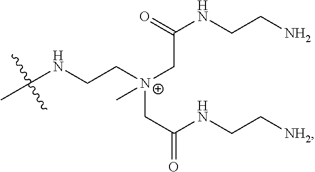 [SMILES: *NCC[N+](C)(CC(=O)NCCN)CC(=O)NCCN]

charge balanced with a counterion (e.g., described herein) as necessary. In some embodiments, T^{A} can be

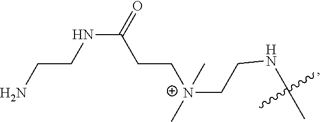 [SMILES: *NCC[N+](C)(C)CCC(=O)NCCN]

charge balanced with a counterion (e.g., described herein) as necessary. In some embodiments, T^{A} can be

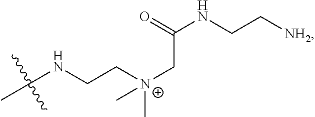 [SMILES: *NCC[N+](C)(C)CC(=O)NCCN]

charge balanced with a counterion (e.g., described herein) as necessary. In some embodiments, T^{A} can be

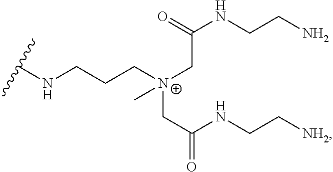 [SMILES: *NCCC[N+](C)(CC(=O)NCCN)CC(=O)NCCN]

charge balanced with a counterion (e.g., described herein) as necessary. In some embodiments, T^{A} can be

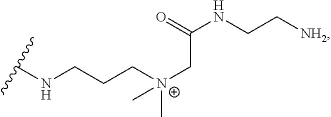 [SMILES: *NCCC[N+](C)(C)CC(=O)NCCN]

charge balanced with a counterion (e.g., described herein) as necessary. In some embodiments, T^{A} can be

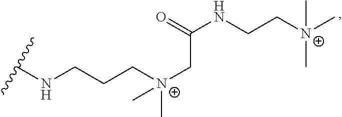 [SMILES: *NCCC[N+](C)(C)CC(=O)NCC[N+](C)(C)C]

charge balanced with a counterion (e.g., described herein) as necessary. In some embodiments, T^{A} can be

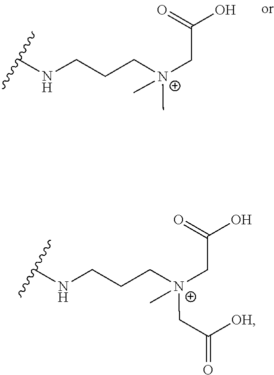 [SMILES: *NCCC[N+](C)(C)CC(=O)O ; *NCCC[N+](C)(CC(=O)O)CC(=O)O]

charge balanced with a counterion (e.g., described herein) as necessary. In some embodiments, T^{A} can be

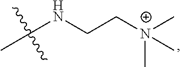 [SMILES: *NCC[N+](C)(C)C]

charge balanced with a counterion (e.g., described herein) as necessary. In some embodiments, T^{A} can be

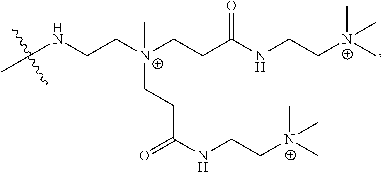 [SMILES: *NCC[N+](C)(CCC(=O)NCC[N+](C)(C)C)CCC(=O)NCC[N+](C)(C)C]

charge balanced with a counterion (e.g., described herein) as necessary. In some embodiments, T^{A} can be

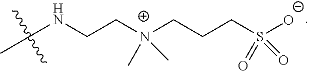 [SMILES: *NCC[N+](C)(C)CCCS(=O)(=O)[O-]]

In some preferred embodiments, the present disclosure also provides a compound of Formula III-4-A or III-5-A, or a pharmaceutically acceptable salt or ester thereof:

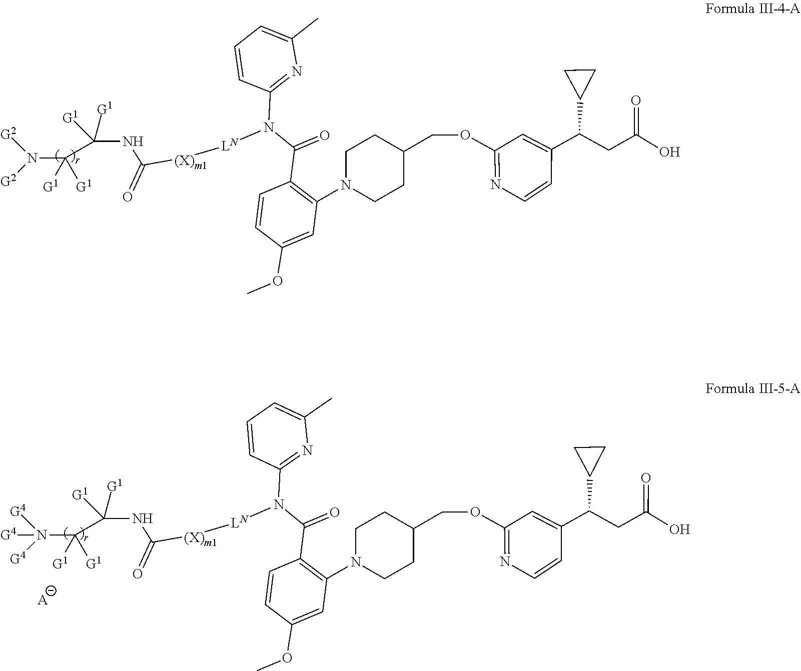

wherein X, m1, L^{N}, G¹, G², G⁴, r, and A⁻ are defined herein, which include any of those described and preferred herein in connection with Formula III (e.g., III-1, III-4, III-5, III-1-A, III-1-A-1, etc.), in any combinations. For example, in some embodiments, L^{N} is a branched or straight chained C₁₋₆ alkyl, such as

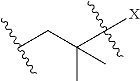

(X is shown to show direction of connection). In some embodiments, L^{N} is null. In some embodiments, X at each occurrence can be independently CR₂, —C(R)—C(R)—,

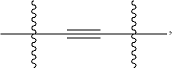 [SMILES: *C#C*]

SiR₂, C(O), O, NR, S, SO₂, or a ring structure, preferably 3-10 membered ring structure, provided that the X group that is directly bonded with L^{N}(when L^{N} is null, it should be understood that the covalent bond is formed with the amide nitrogen atom in Formula III-4-A or III-5-A) is CR₂ or a ring structure, preferably 3-10 membered ring structure, more preferably CR₂, such as CH₂, wherein R at each occurrence is independently hydrogen, halogen, optionally substituted C₁₋₄ alkyl, or optionally substituted C₁₋₄ alkoxy, typically hydrogen or C₁₋₄ alkyl. In some embodiments, the integer m1 can be 12-50, such as 14-50, 16-30, etc. Preferably, the total number of non-hydrogen atoms of the (X)ₘ₁ moiety is between 15-50, such as 18, 20, 25, 30, 35, 40, 45, or 50, or any ranges between the recited value. In some preferred embodiments, none of the X groups is a ring structure. In some preferred embodiments, one instance of X is a ring structure, preferably 3-10 membered ring structure. For example, in some embodiments, one instance of X is triazole and the remaining X groups are each independently CR₂, —C(R)═C(R)—,

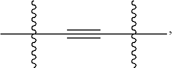 [SMILES: *C#C*]

or O, more preferably, CR₂ or O, wherein R at each occurrence is independently hydrogen or C₁₋₄ alkyl. In some embodiments, none of the X groups is a ring structure, and each X is independently CR₂, —C(R)═C(R)—,

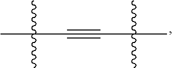 [SMILES: *C#C*]

or O, more preferably, CR₂ or O, wherein R at each occurrence is independently hydrogen or C₁₋₄ alkyl. In some embodiments, up to 6 instances (e.g., 1, 2, 3, 4, 5, or 6) of X can be O. In some embodiments, all instances of X is CR₂, wherein R at each occurrence is independently hydrogen or C₁₋₄ alkyl (preferably, methyl). In some embodiments, one or two instances of X is O, and all other instances of X is CR₂, wherein R at each occurrence is independently hydrogen or C₁₋₄ alkyl (preferably, methyl). In some embodiments, one or two instances of X is O, one X is triazole, and all other instances of X is CR₂, wherein R at each occurrence is independently hydrogen or C₁₋₄ alkyl (preferably, methyl). In some embodiments, R is hydrogen. The definition and preferred definition of G¹, G², G⁴, r, and A⁻ are also described herein, including any of those corresponding definitions shown in compounds according to any of the compounds listed in Table 1 herein or any of the compound according to Examples 1-221.

In some embodiments, the present disclosure also provides a compound selected from Table 1 below, or a pharmaceutically acceptable salt or ester thereof, wherein A represents a counterion, preferably, a pharmaceutically acceptable anion, such as Cl⁻, etc. It should be clear that under certain conditions, the compound shown in Table 1 may exist as an internal salt, i.e., a zwitterion structure, in which case, A may not be needed for balancing the charge shown. As used herein, an internal salt derivable from a compound shown in Table 1 (with or without A⁻) is within the definition of the compound shown in Table 1, or a pharmaceutically acceptable salt thereof.

TABLE 1
List of Exemplary Compounds

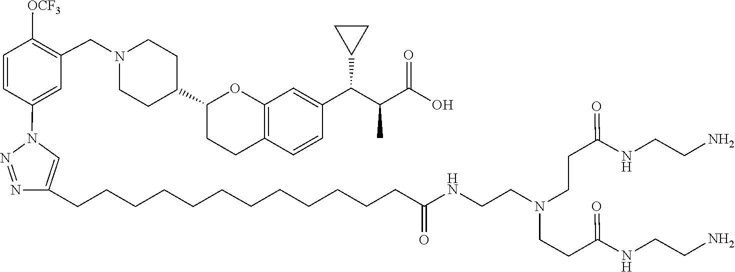 [SMILES: C[C@H](C(=O)O)[C@H](c1ccc2c(c1)O[C@@H](C1CCN(Cc3cc(-n4cc(CCCCCCCCCCCCC(=O)NCCN(CCC(=O)NCCN)CCC(=O)NCCN)nn4)ccc3OC(F)(F)F)CC1)CC2)C1CC1]

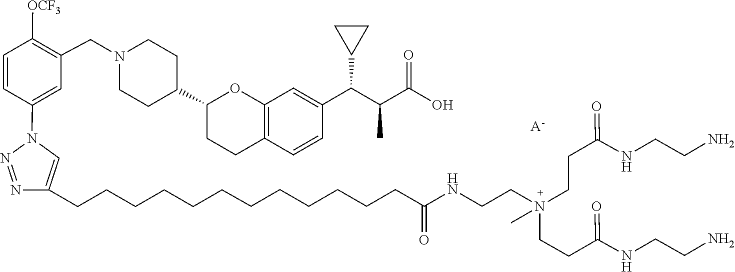

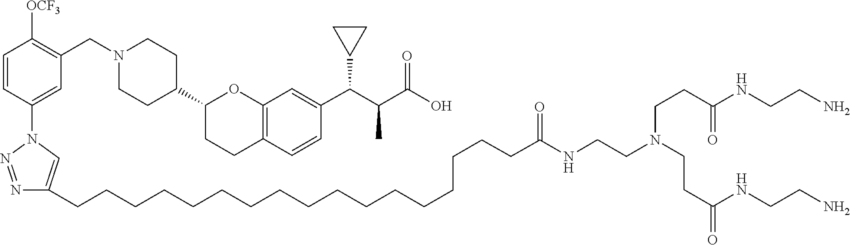 [SMILES: C[C@H](C(=O)O)[C@H](c1ccc2c(c1)O[C@@H](C1CCN(Cc3cc(-n4cc(CCCCCCCCCCCCCCCCCC(=O)NCCN(CCC(=O)NCCN)CCC(=O)NCCN)nn4)ccc3OC(F)(F)F)CC1)CC2)C1CC1]

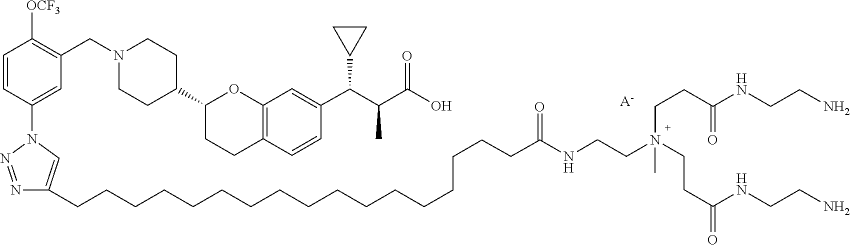

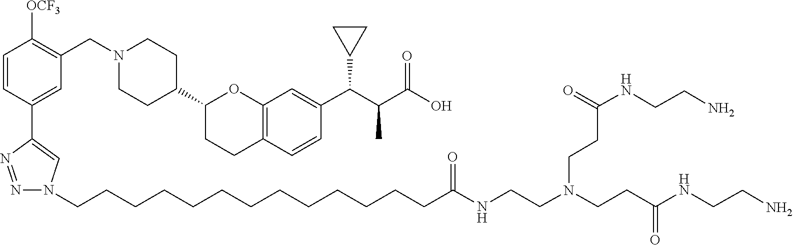 [SMILES: C[C@H](C(=O)O)[C@H](c1ccc2c(c1)O[C@@H](C1CCN(Cc3cc(-c4cn(CCCCCCCCCCCCCC(=O)NCCN(CCC(=O)NCCN)CCC(=O)NCCN)nn4)ccc3OC(F)(F)F)CC1)CC2)C1CC1]

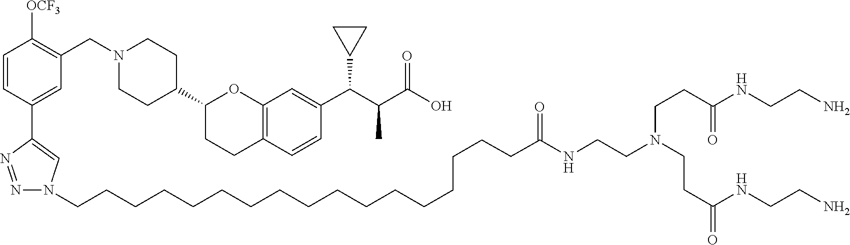 [SMILES: C[C@H](C(=O)O)[C@H](c1ccc2c(c1)O[C@@H](C1CCN(Cc3cc(-c4cn(CCCCCCCCCCCCCCCCCC(=O)NCCN(CCC(=O)NCCN)CCC(=O)NCCN)nn4)ccc3OC(F)(F)F)CC1)CC2)C1CC1]

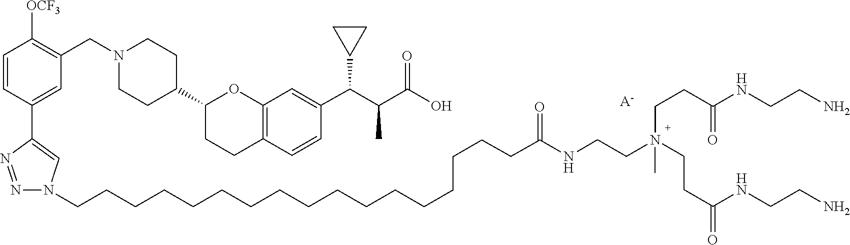

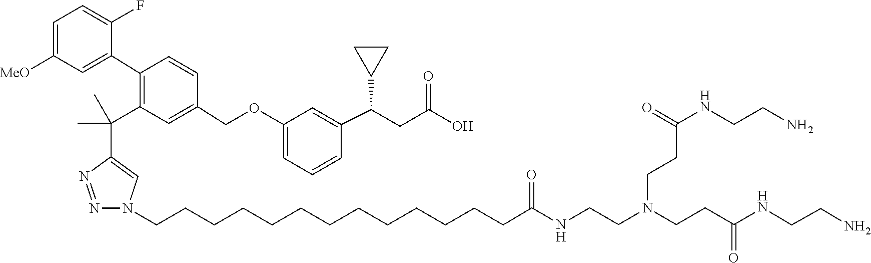 [SMILES: COc1ccc(F)c(-c2ccc(COc3cccc([C@@H](CC(=O)O)C4CC4)c3)cc2C(C)(C)c2cn(CCCCCCCCCCCCCC(=O)NCCN(CCC(=O)NCCN)CCC(=O)NCCN)nn2)c1]

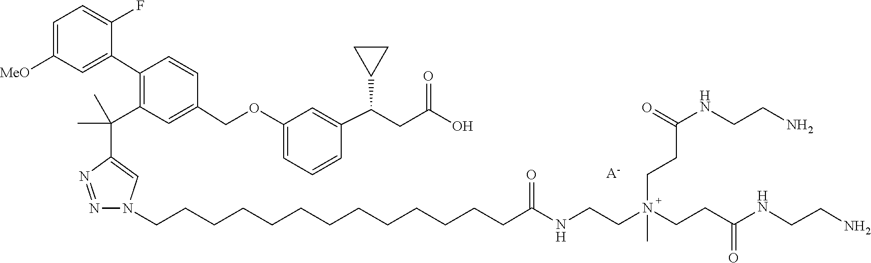

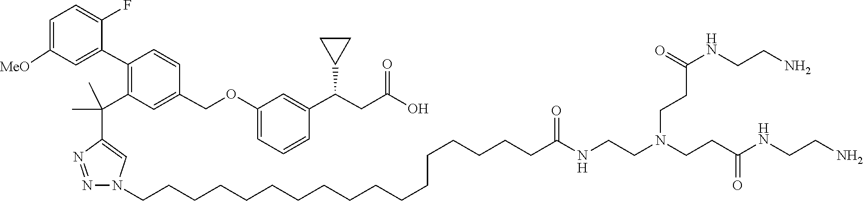 [SMILES: COc1ccc(F)c(-c2ccc(COc3cccc([C@@H](CC(=O)O)C4CC4)c3)cc2C(C)(C)c2cn(CCCCCCCCCCCCCCCCCC(=O)NCCN(CCC(=O)NCCN)CCC(=O)NCCN)nn2)c1]

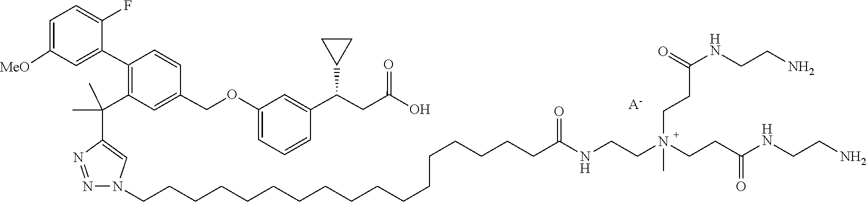

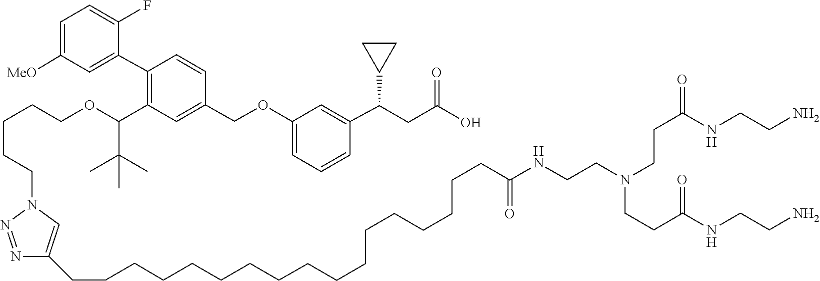 [SMILES: COc1ccc(F)c(-c2ccc(COc3cccc([C@@H](CC(=O)O)C4CC4)c3)cc2C(OCCCCCn2cc(CCCCCCCCCCCCCCCCCC(=O)NCCN(CCC(=O)NCCN)CCC(=O)NCCN)nn2)C(C)(C)C)c1]

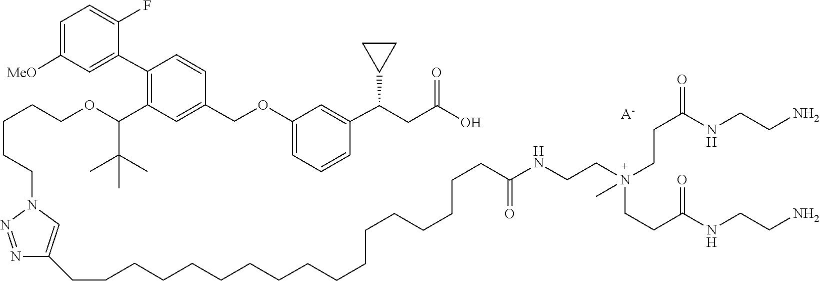

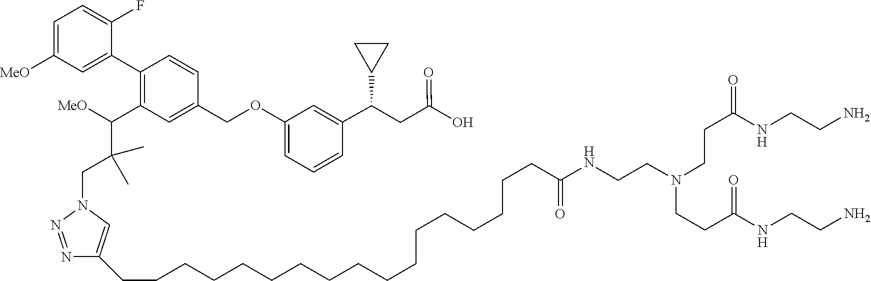 [SMILES: COc1ccc(F)c(-c2ccc(COc3cccc([C@@H](CC(=O)O)C4CC4)c3)cc2C(OC)C(C)(C)Cn2cc(CCCCCCCCCCCCCCCCCC(=O)NCCN(CCC(=O)NCCN)CCC(=O)NCCN)nn2)c1]

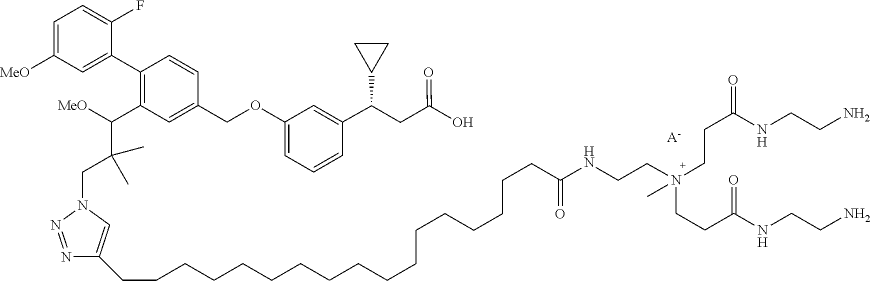

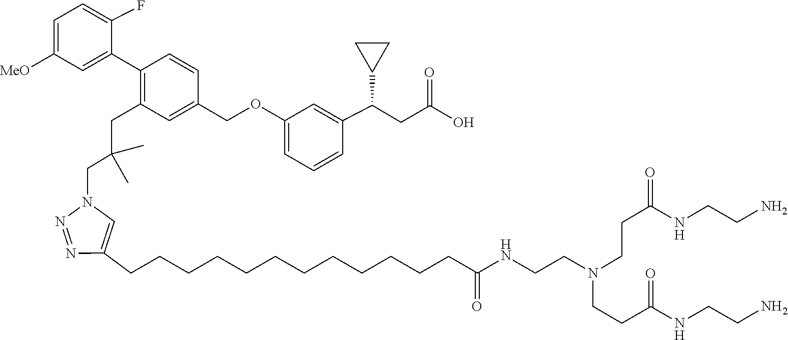 [SMILES: COc1ccc(F)c(-c2ccc(COc3cccc([C@@H](CC(=O)O)C4CC4)c3)cc2CC(C)(C)Cn2cc(CCCCCCCCCCCCC(=O)NCCN(CCC(=O)NCCN)CCC(=O)NCCN)nn2)c1]

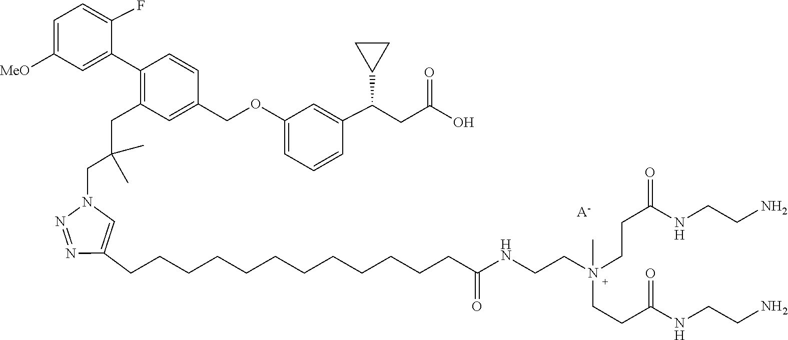

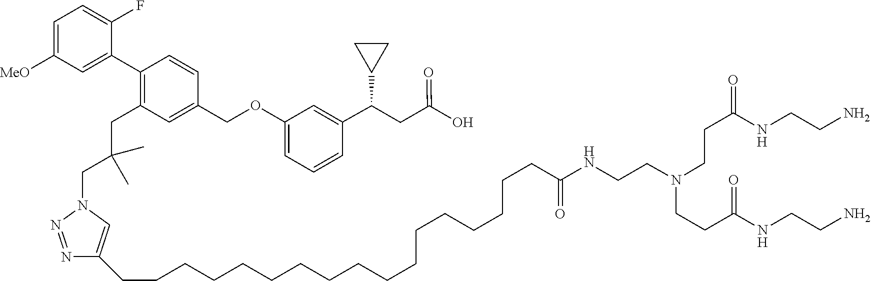 [SMILES: COc1ccc(F)c(-c2ccc(COc3cccc([C@@H](CC(=O)O)C4CC4)c3)cc2CC(C)(C)Cn2cc(CCCCCCCCCCCCCCCCCC(=O)NCCN(CCC(=O)NCCN)CCC(=O)NCCN)nn2)c1]

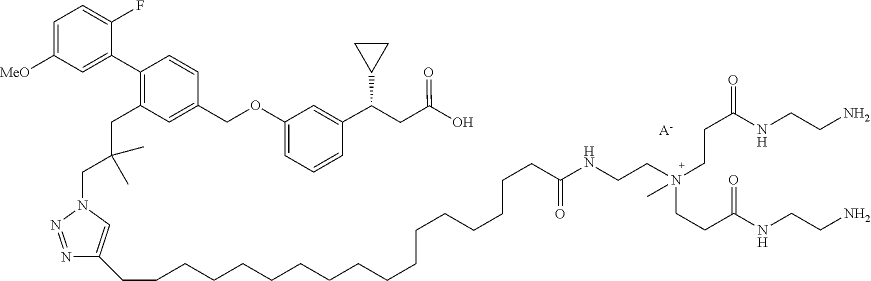

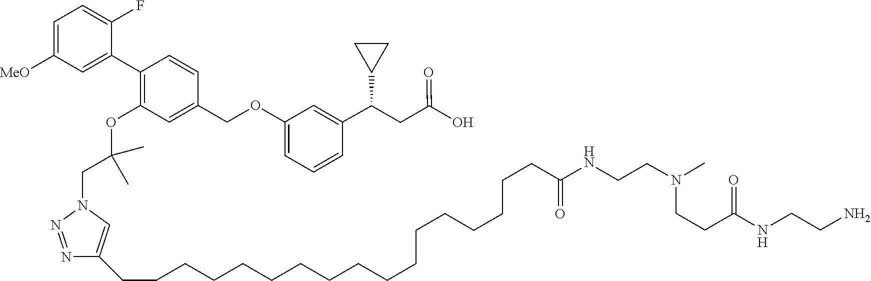 [SMILES: COc1ccc(F)c(-c2ccc(COc3cccc([C@@H](CC(=O)O)C4CC4)c3)cc2OC(C)(C)Cn2cc(CCCCCCCCCCCCCCCCCC(=O)NCCN(C)CCC(=O)NCCN)nn2)c1]

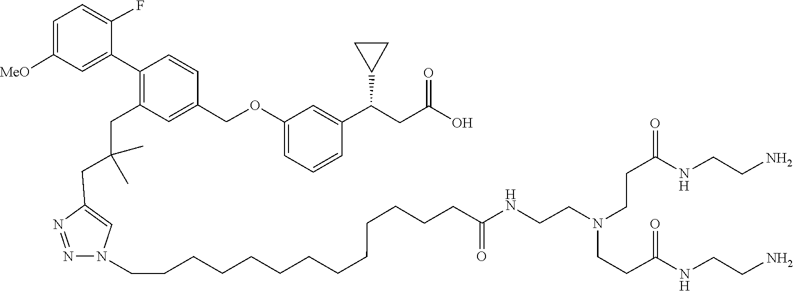 [SMILES: COc1ccc(F)c(-c2ccc(COc3cccc([C@@H](CC(=O)O)C4CC4)c3)cc2CC(C)(C)Cc2cn(CCCCCCCCCCCCCC(=O)NCCN(CCC(=O)NCCN)CCC(=O)NCCN)nn2)c1]

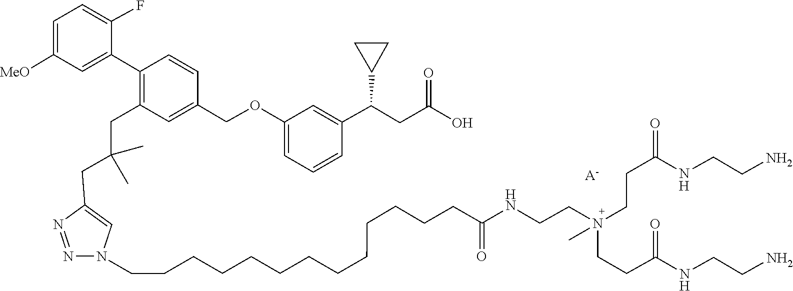

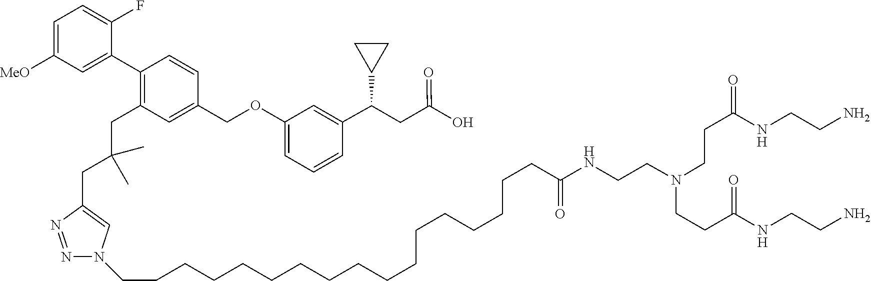 [SMILES: COc1ccc(F)c(-c2ccc(COc3cccc([C@@H](CC(=O)O)C4CC4)c3)cc2CC(C)(C)Cc2cn(CCCCCCCCCCCCCCCCCC(=O)NCCN(CCC(=O)NCCN)CCC(=O)NCCN)nn2)c1]

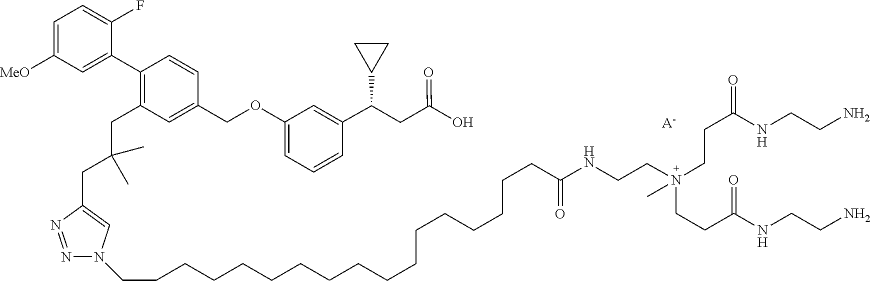

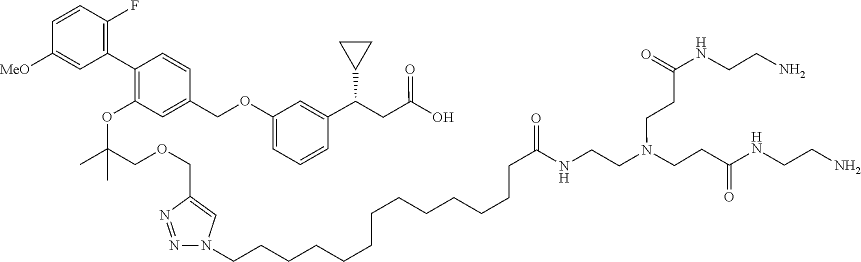 [SMILES: COc1ccc(F)c(-c2ccc(COc3cccc([C@@H](CC(=O)O)C4CC4)c3)cc2OC(C)(C)COCc2cn(CCCCCCCCCCCCCC(=O)NCCN(CCC(=O)NCCN)CCC(=O)NCCN)nn2)c1]

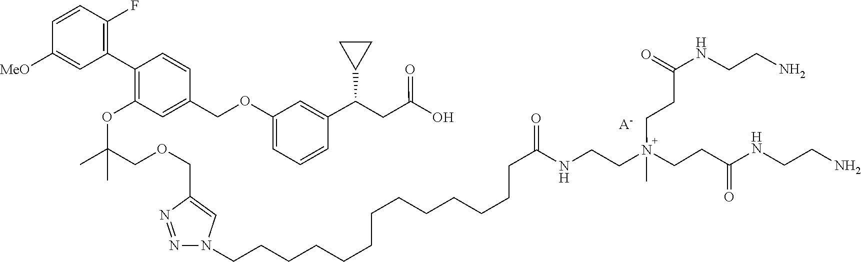

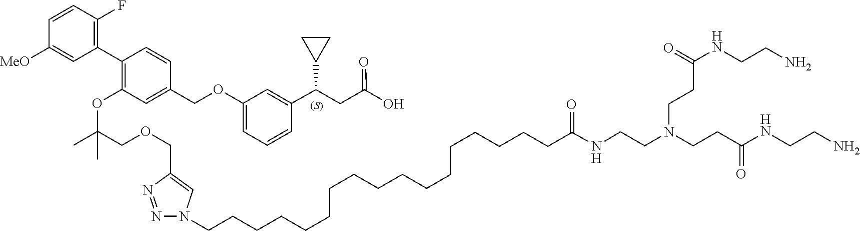

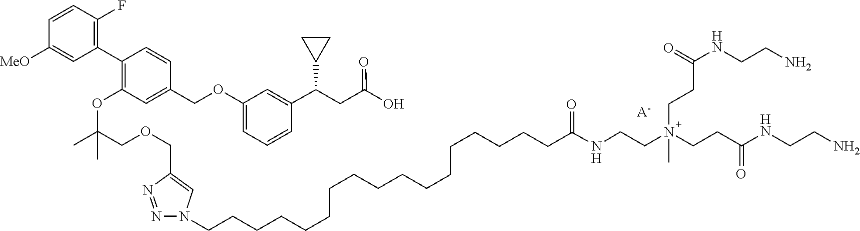

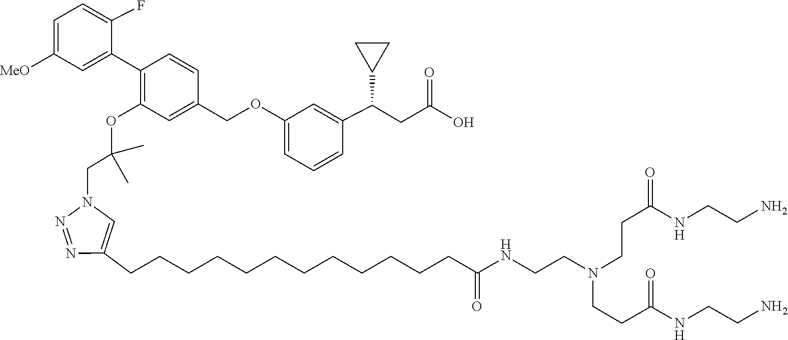 [SMILES: COc1ccc(F)c(-c2ccc(COc3cccc([C@@H](CC(=O)O)C4CC4)c3)cc2OC(C)(C)Cn2cc(CCCCCCCCCCCCC(=O)NCCN(CCC(=O)NCCN)CCC(=O)NCCN)nn2)c1]

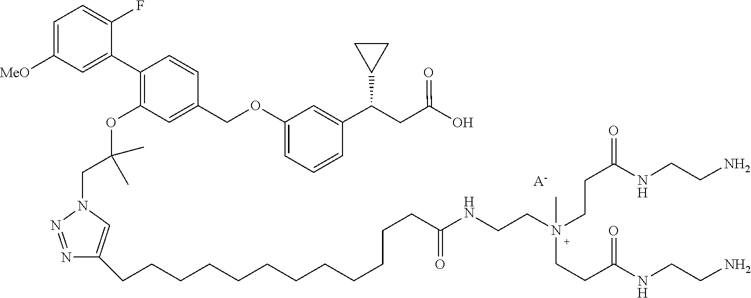

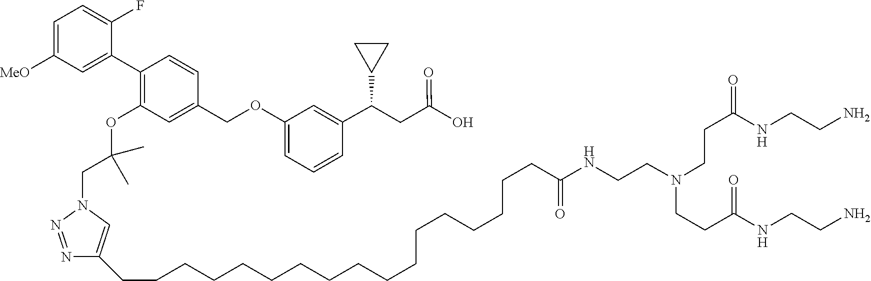 [SMILES: COc1ccc(F)c(-c2ccc(COc3cccc([C@@H](CC(=O)O)C4CC4)c3)cc2OC(C)(C)Cn2cc(CCCCCCCCCCCCCCCCCC(=O)NCCN(CCC(=O)NCCN)CCC(=O)NCCN)nn2)c1]

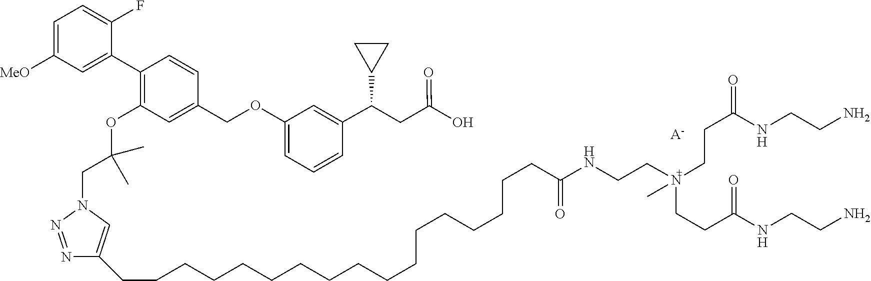

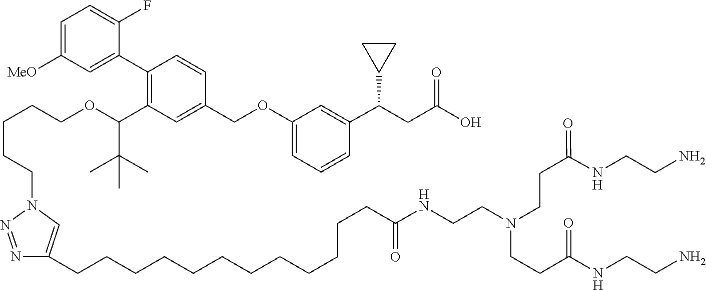 [SMILES: COc1ccc(F)c(-c2ccc(COc3cccc([C@@H](CC(=O)O)C4CC4)c3)cc2C(OCCCCCn2cc(CCCCCCCCCCCCC(=O)NCCN(CCC(=O)NCCN)CCC(=O)NCCN)nn2)C(C)(C)C)c1]

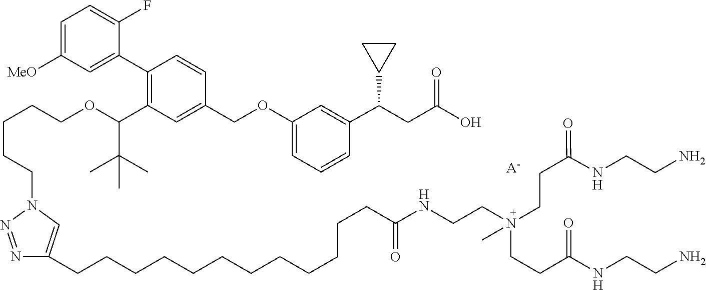

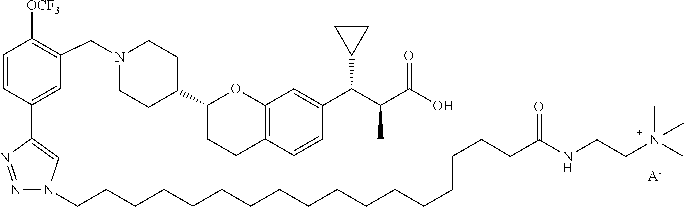

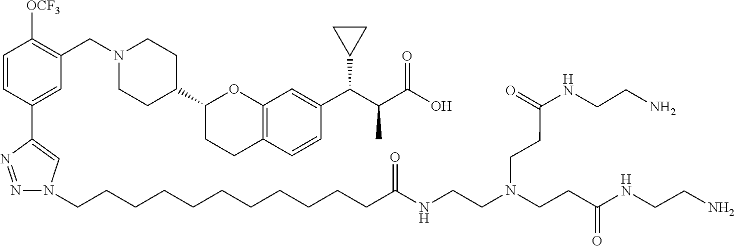 [SMILES: C[C@H](C(=O)O)[C@H](c1ccc2c(c1)O[C@@H](C1CCN(Cc3cc(-c4cn(CCCCCCCCCCCC(=O)NCCN(CCC(=O)NCCN)CCC(=O)NCCN)nn4)ccc3OC(F)(F)F)CC1)CC2)C1CC1]

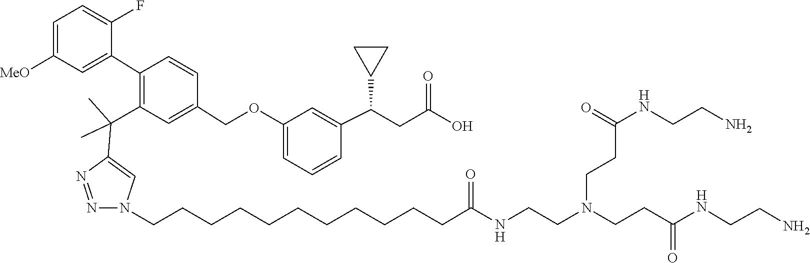 [SMILES: COc1ccc(F)c(-c2ccc(COc3cccc([C@@H](CC(=O)O)C4CC4)c3)cc2C(C)(C)c2cn(CCCCCCCCCCCC(=O)NCCN(CCC(=O)NCCN)CCC(=O)NCCN)nn2)c1]

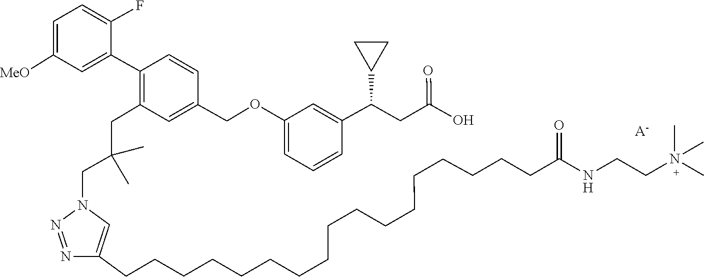

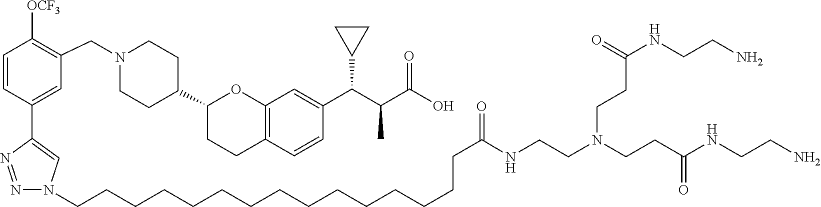 [SMILES: C[C@H](C(=O)O)[C@H](c1ccc2c(c1)O[C@@H](C1CCN(Cc3cc(-c4cn(CCCCCCCCCCCCCCCC(=O)NCCN(CCC(=O)NCCN)CCC(=O)NCCN)nn4)ccc3OC(F)(F)F)CC1)CC2)C1CC1]

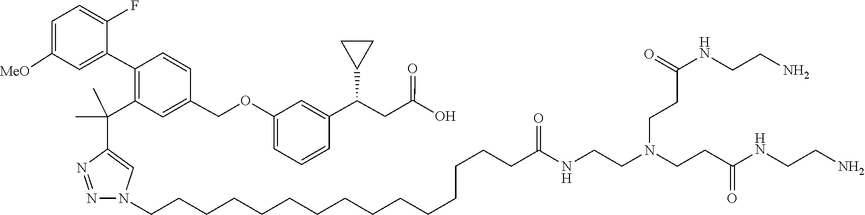 [SMILES: COc1ccc(F)c(-c2ccc(COc3cccc([C@@H](CC(=O)O)C4CC4)c3)cc2C(C)(C)c2cn(CCCCCCCCCCCCCCCC(=O)NCCN(CCC(=O)NCCN)CCC(=O)NCCN)nn2)c1]

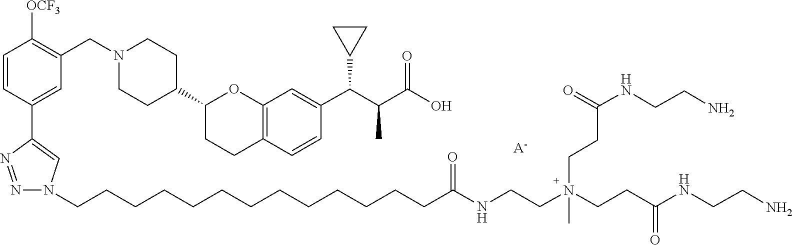

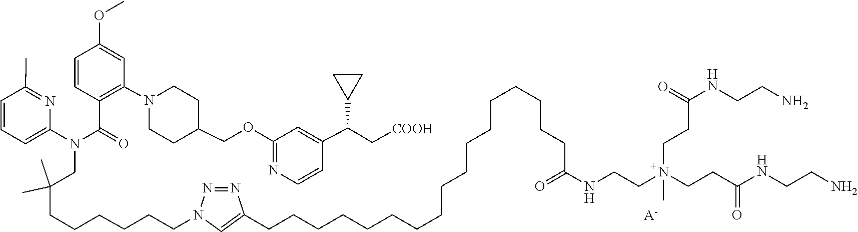

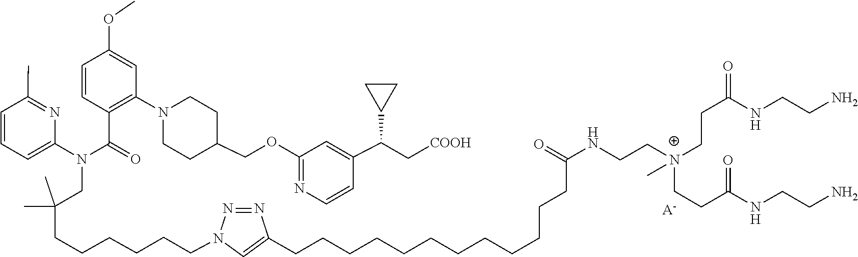

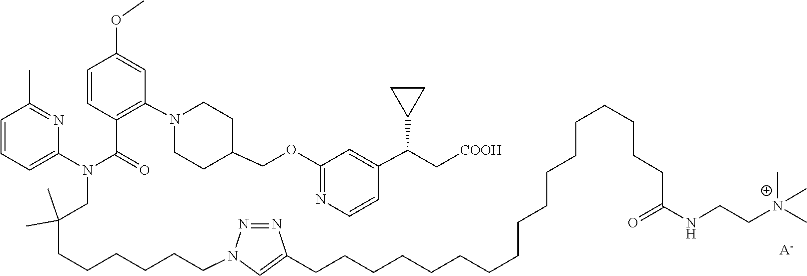

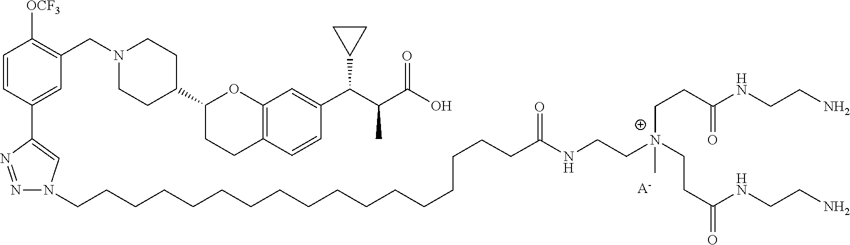

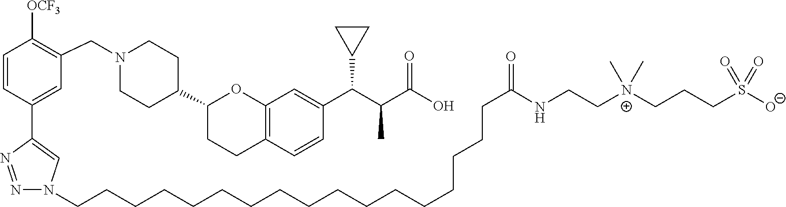 [SMILES: C[C@H](C(=O)O)[C@H](c1ccc2c(c1)O[C@@H](C1CCN(Cc3cc(-c4cn(CCCCCCCCCCCCCCCCCC(=O)NCC[N+](C)(C)CCCS(=O)(=O)[O-])nn4)ccc3OC(F)(F)F)CC1)CC2)C1CC1]

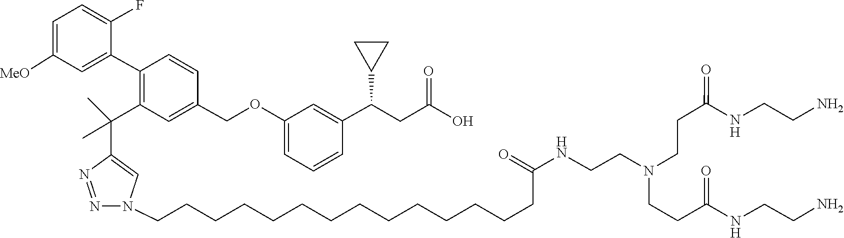 [SMILES: COc1ccc(F)c(-c2ccc(COc3cccc([C@@H](CC(=O)O)C4CC4)c3)cc2C(C)(C)c2cn(CCCCCCCCCCCCCCC(=O)NCCN(CCC(=O)NCCN)CCC(=O)NCCN)nn2)c1]

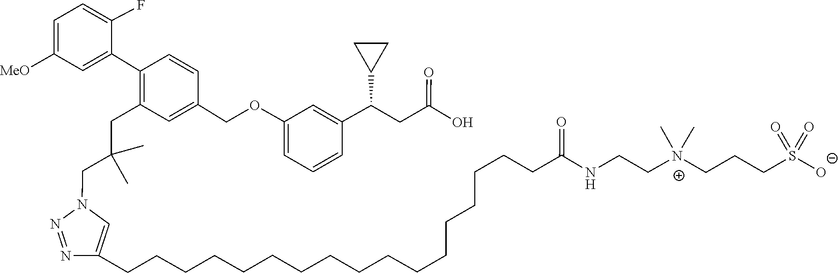 [SMILES: COc1ccc(F)c(-c2ccc(COc3cccc([C@@H](CC(=O)O)C4CC4)c3)cc2CC(C)(C)Cn2cc(CCCCCCCCCCCCCCCCCC(=O)NCC[N+](C)(C)CCCS(=O)(=O)[O-])nn2)c1]

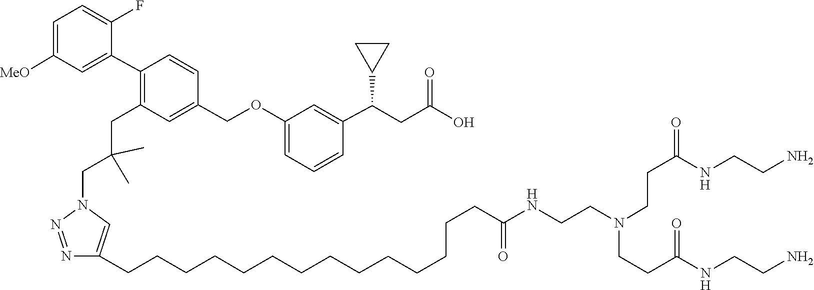 [SMILES: COc1ccc(F)c(-c2ccc(COc3cccc([C@@H](CC(=O)O)C4CC4)c3)cc2CC(C)(C)Cn2cc(CCCCCCCCCCCCCCC(=O)NCCN(CCC(=O)NCCN)CCC(=O)NCCN)nn2)c1]

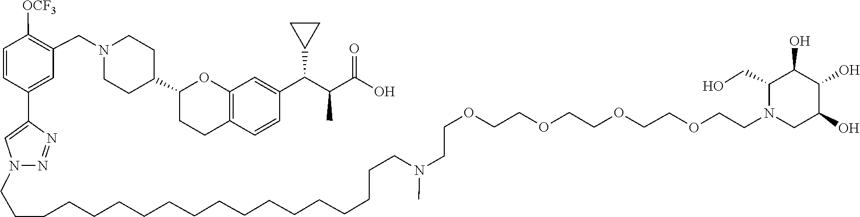 [SMILES: C[C@H](C(=O)O)[C@H](c1ccc2c(c1)O[C@@H](C1CCN(Cc3cc(-c4cn(CCCCCCCCCCCCCCCCCCN(C)CCOCCOCCOCCOCCN5C[C@H](O)[C@@H](O)[C@H](O)[C@H]5CO)nn4)ccc3OC(F)(F)F)CC1)CC2)C1CC1]

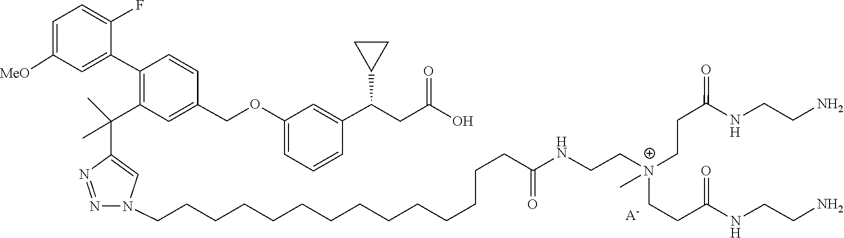

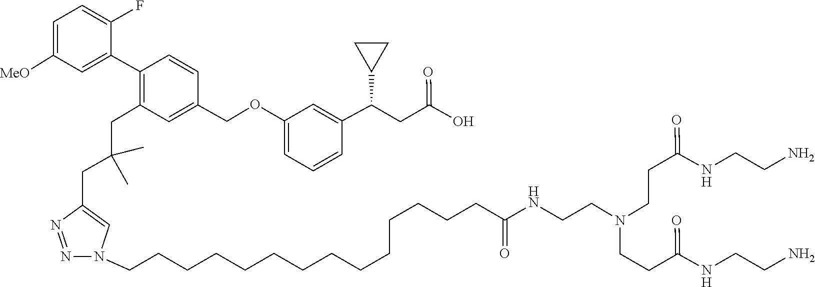 [SMILES: COc1ccc(F)c(-c2ccc(COc3cccc([C@@H](CC(=O)O)C4CC4)c3)cc2CC(C)(C)Cc2cn(CCCCCCCCCCCCCCC(=O)NCCN(CCC(=O)NCCN)CCC(=O)NCCN)nn2)c1]

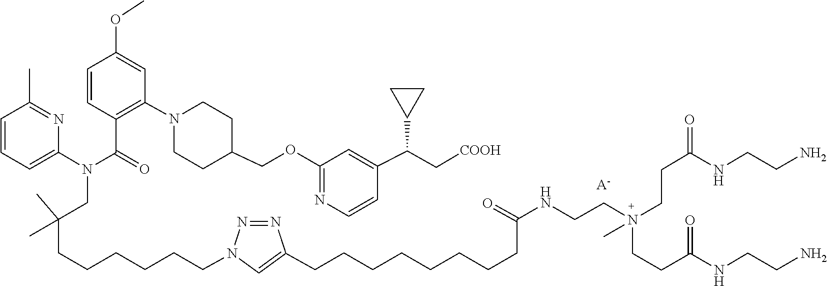

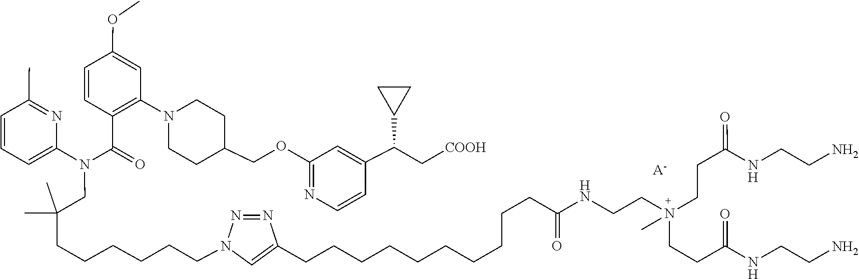

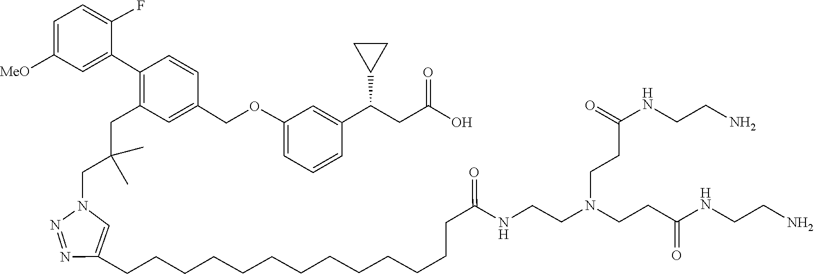 [SMILES: COc1ccc(F)c(-c2ccc(COc3cccc([C@@H](CC(=O)O)C4CC4)c3)cc2CC(C)(C)Cn2cc(CCCCCCCCCCCCCC(=O)NCCN(CCC(=O)NCCN)CCC(=O)NCCN)nn2)c1]

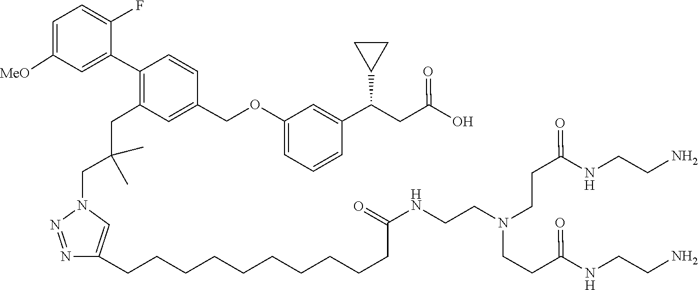 [SMILES: COc1ccc(F)c(-c2ccc(COc3cccc([C@@H](CC(=O)O)C4CC4)c3)cc2CC(C)(C)Cn2cc(CCCCCCCCCCC(=O)NCCN(CCC(=O)NCCN)CCC(=O)NCCN)nn2)c1]

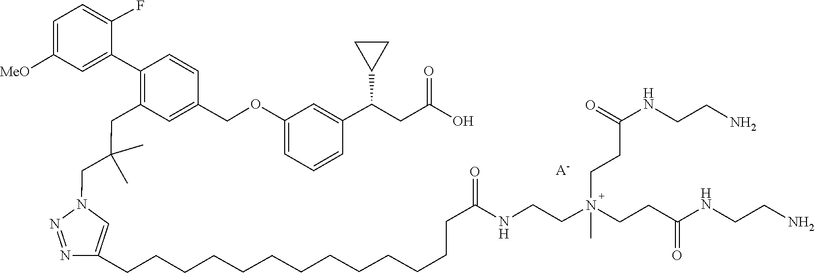

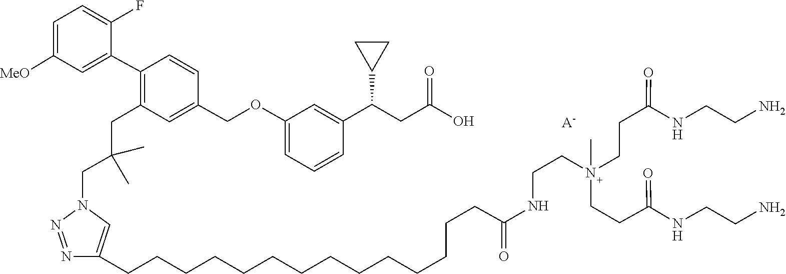

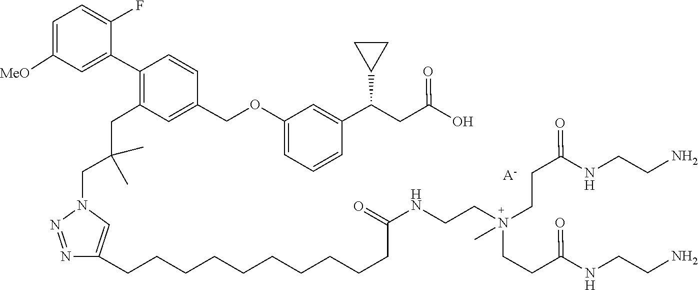

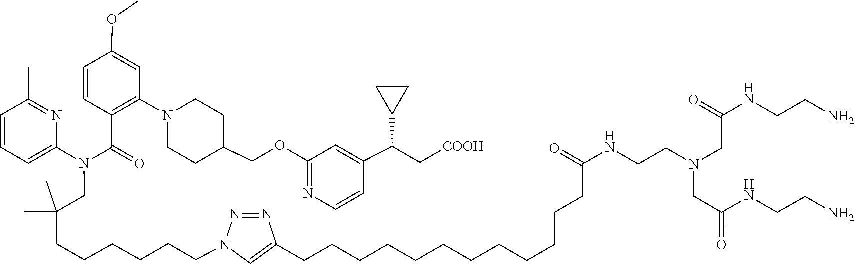 [SMILES: COc1ccc(C(=O)N(CC(C)(C)CCCCCCn2cc(CCCCCCCCCCCCC(=O)NCCN(CC(=O)NCCN)CC(=O)NCCN)nn2)c2cccc(C)n2)c(N2CCC(COc3cc([C@@H](CC(=O)O)C4CC4)ccn3)CC2)c1]

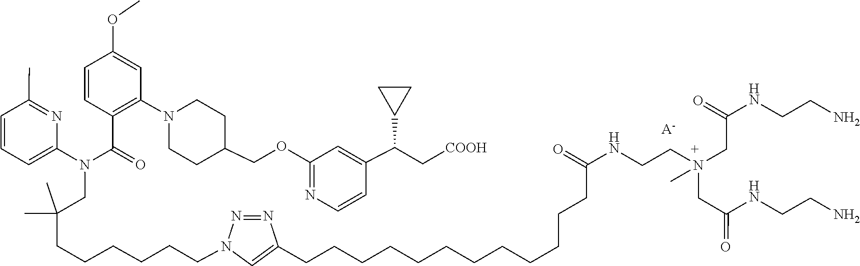

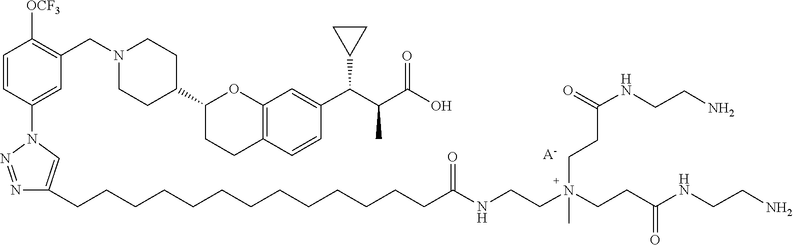

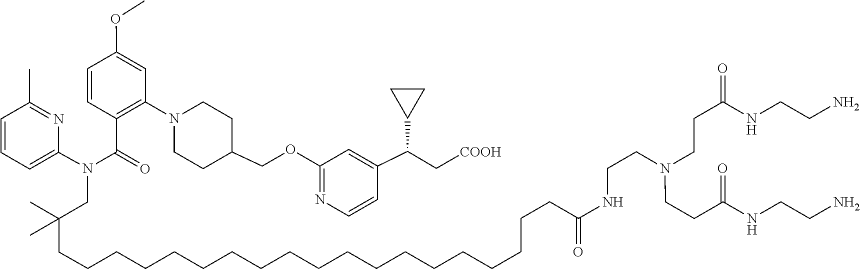 [SMILES: COc1ccc(C(=O)N(CC(C)(C)CCCCCCCCCCCCCCCCCCCC(=O)NCCN(CCC(=O)NCCN)CCC(=O)NCCN)c2cccc(C)n2)c(N2CCC(COc3cc([C@@H](CC(=O)O)C4CC4)ccn3)CC2)c1]

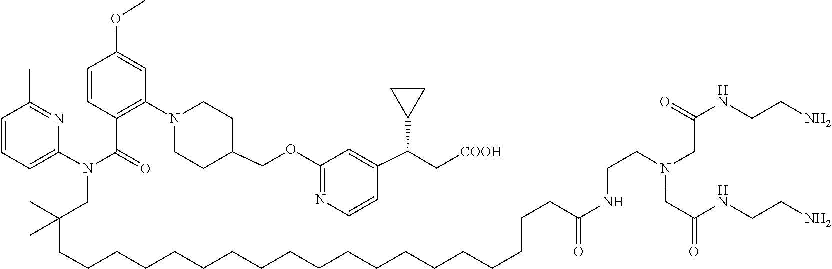 [SMILES: COc1ccc(C(=O)N(CC(C)(C)CCCCCCCCCCCCCCCCCCCC(=O)NCCN(CC(=O)NCCN)CC(=O)NCCN)c2cccc(C)n2)c(N2CCC(COc3cc([C@@H](CC(=O)O)C4CC4)ccn3)CC2)c1]

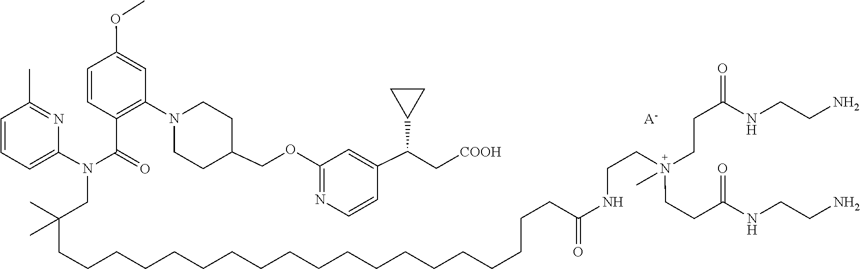

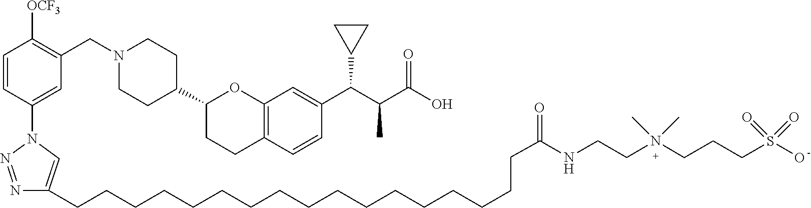

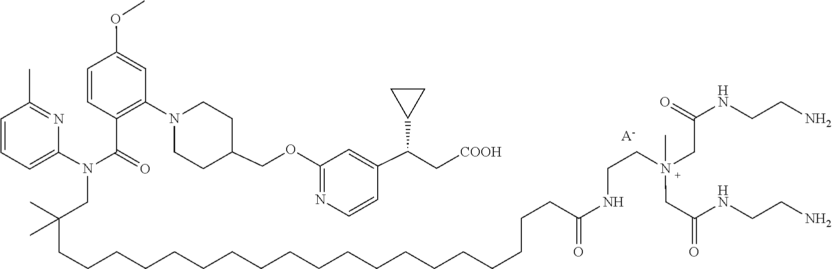

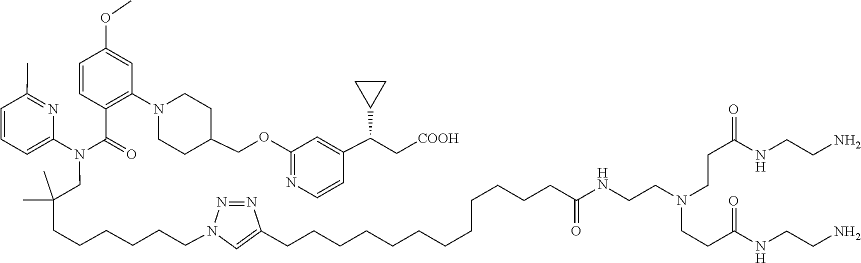 [SMILES: COc1ccc(C(=O)N(CC(C)(C)CCCCCCn2cc(CCCCCCCCCCCCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

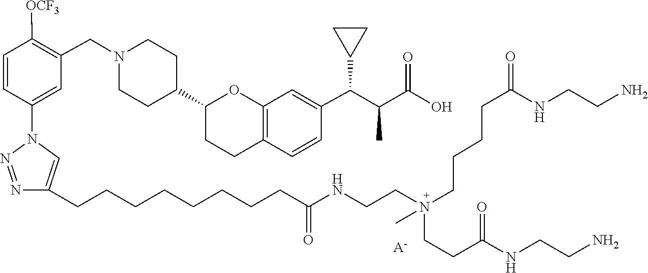

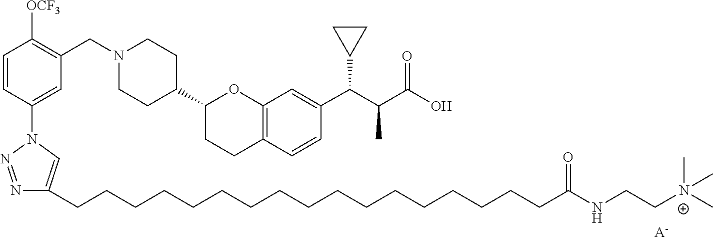

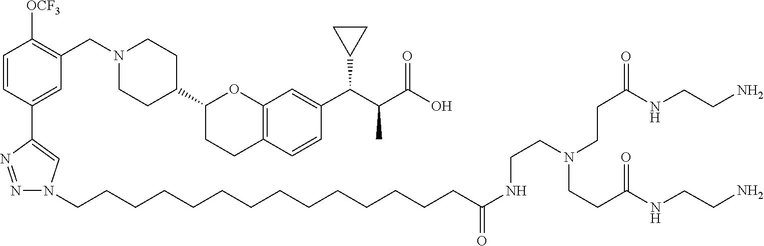 [SMILES: C[C@H](C(=O)O)[C@H](c1ccc2c(c1)O[C@@H](C1CCN(Cc3cc(-c4cn(CCCCCCCCCCCCCCC(=O)NCCN(CCC(=O)NCCN)CCC(=O)NCCN)nn4)ccc3OC(F)(F)F)CC1)CC2)C1CC1]

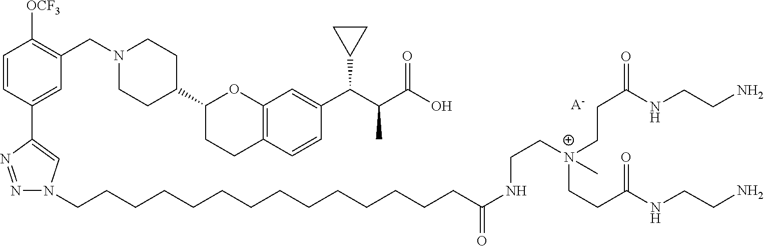

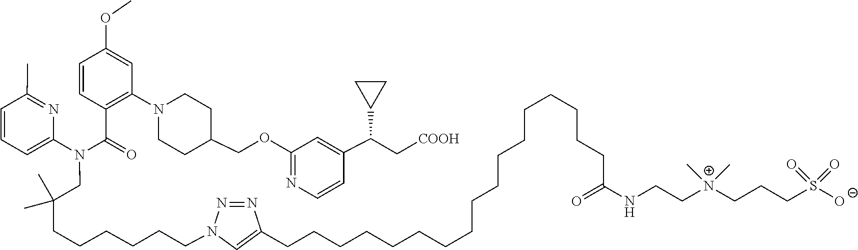 [SMILES: COc1ccc(C(=O)N(CC(C)(C)CCCCCCn2cc(CCCCCCCCCCCCCCCCCC(=O)NCC[N+](C)(C)CCCS(=O)(=O)[O-])nn2)c2cccc(C)n2)c(N2CCC(COc3cc([C@@H](CC(=O)O)C4CC4)ccn3)CC2)c1]

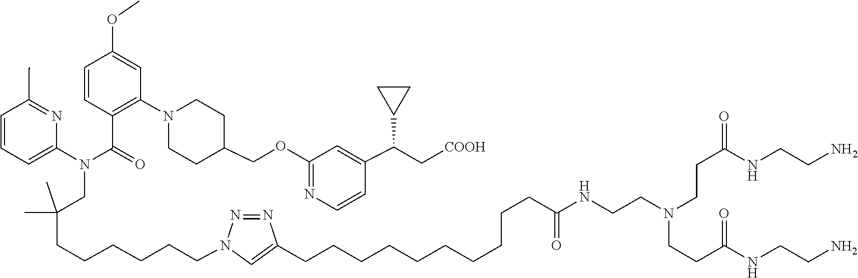 [SMILES: COc1ccc(C(=O)N(CC(C)(C)CCCCCCn2cc(CCCCCCCCCCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

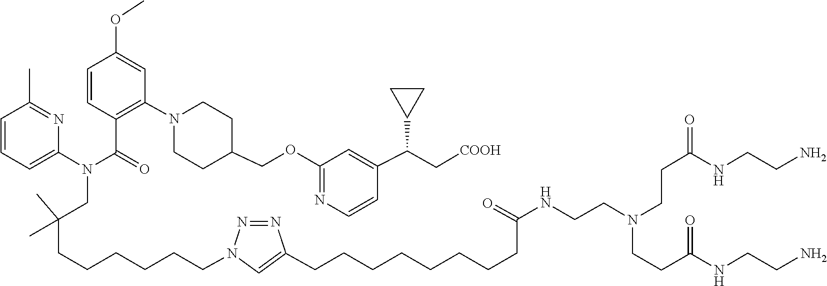 [SMILES: COc1ccc(C(=O)N(CC(C)(C)CCCCCCn2cc(CCCCCCCCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

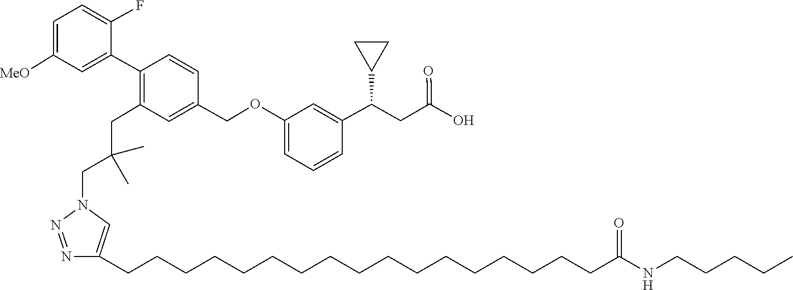 [SMILES: CCCCCNC(=O)CCCCCCCCCCCCCCCCCc1cn(CC(C)(C)Cc2cc(COc3cccc([C@@H](CC(=O)O)C4CC4)c3)ccc2-c2cc(OC)ccc2F)nn1]

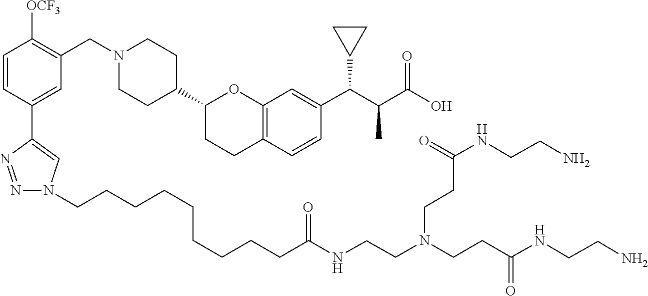 [SMILES: C[C@H](C(=O)O)[C@H](c1ccc2c(c1)O[C@@H](C1CCN(Cc3cc(-c4cn(CCCCCCCCCC(=O)NCCN(CCC(=O)NCCN)CCC(=O)NCCN)nn4)ccc3OC(F)(F)F)CC1)CC2)C1CC1]

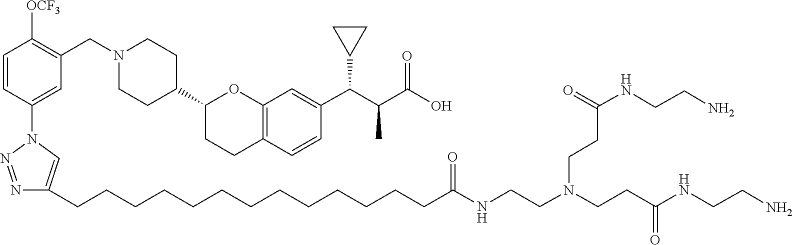 [SMILES: C[C@H](C(=O)O)[C@H](c1ccc2c(c1)O[C@@H](C1CCN(Cc3cc(-n4cc(CCCCCCCCCCCCCC(=O)NCCN(CCC(=O)NCCN)CCC(=O)NCCN)nn4)ccc3OC(F)(F)F)CC1)CC2)C1CC1]

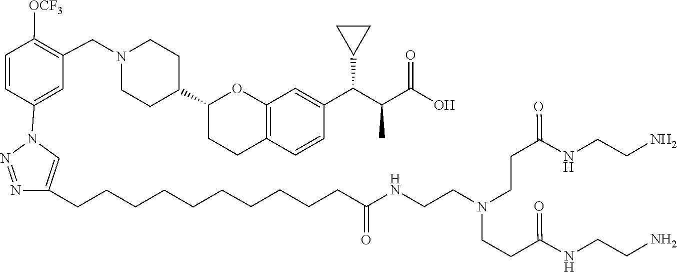 [SMILES: C[C@H](C(=O)O)[C@H](c1ccc2c(c1)O[C@@H](C1CCN(Cc3cc(-n4cc(CCCCCCCCCCC(=O)NCCN(CCC(=O)NCCN)CCC(=O)NCCN)nn4)ccc3OC(F)(F)F)CC1)CC2)C1CC1]

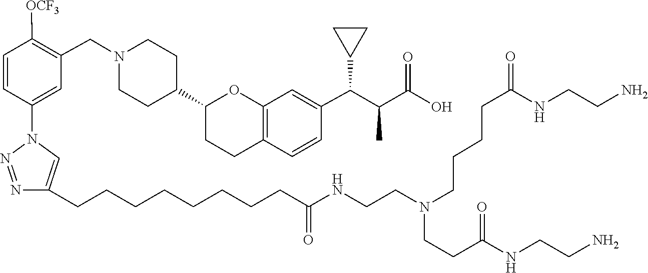 [SMILES: C[C@H](C(=O)O)[C@H](c1ccc2c(c1)O[C@@H](C1CCN(Cc3cc(-n4cc(CCCCCCCCC(=O)NCCN(CCCCC(=O)NCCN)CCC(=O)NCCN)nn4)ccc3OC(F)(F)F)CC1)CC2)C1CC1]

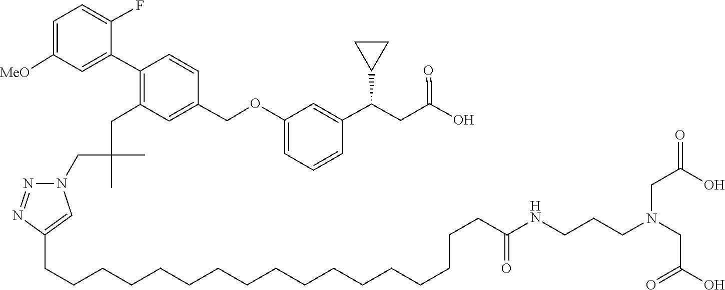 [SMILES: COc1ccc(F)c(-c2ccc(COc3cccc([C@@H](CC(=O)O)C4CC4)c3)cc2CC(C)(C)Cn2cc(CCCCCCCCCCCCCCCCCC(=O)NCCCN(CC(=O)O)CC(=O)O)nn2)c1]

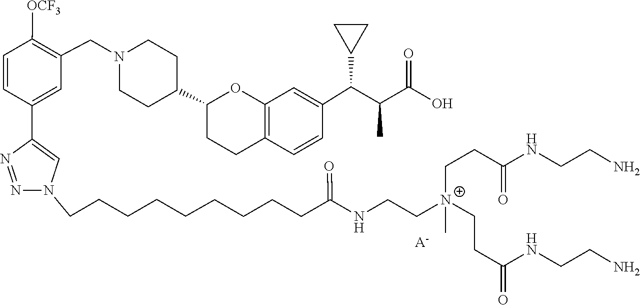

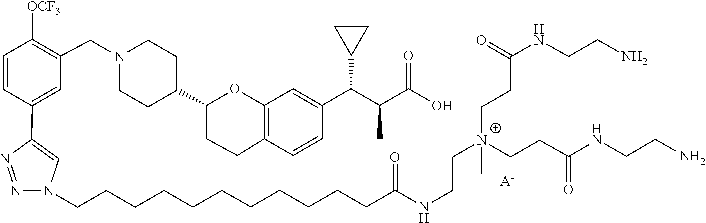

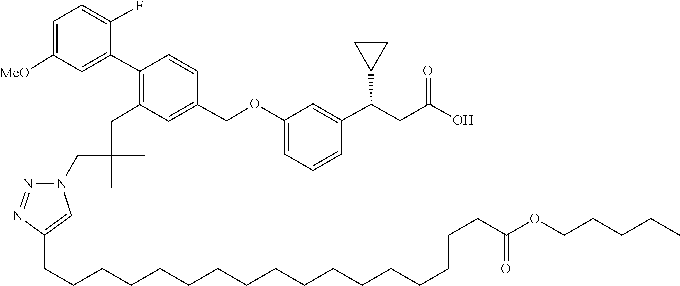 [SMILES: CCCCCOC(=O)CCCCCCCCCCCCCCCCCc1cn(CC(C)(C)Cc2cc(COc3cccc([C@@H](CC(=O)O)C4CC4)c3)ccc2-c2cc(OC)ccc2F)nn1]

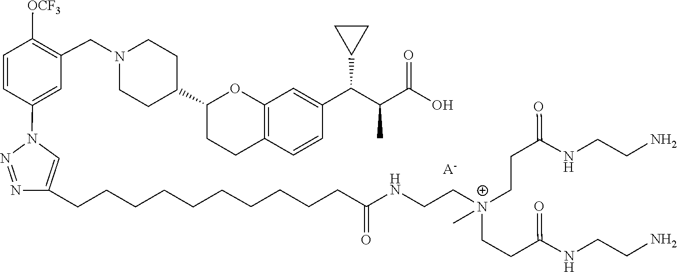

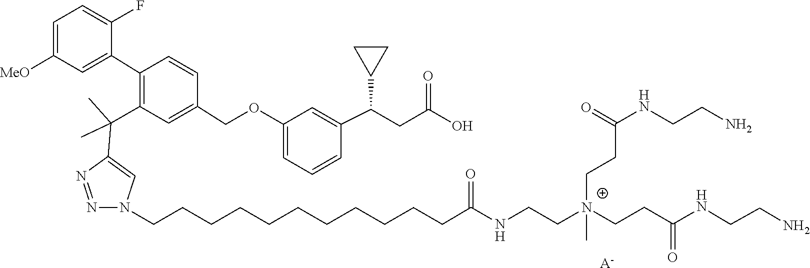

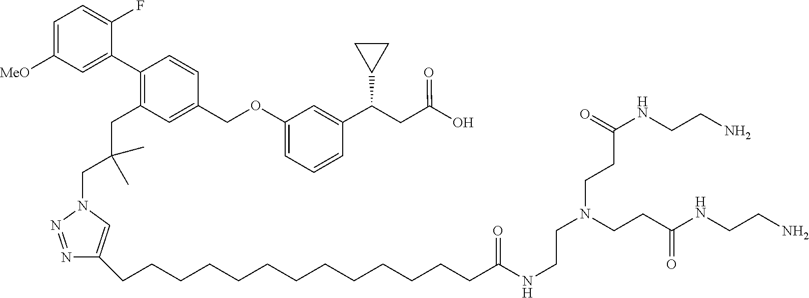 [SMILES: COc1ccc(F)c(-c2ccc(COc3cccc([C@@H](CC(=O)O)C4CC4)c3)cc2CC(C)(C)Cn2cc(CCCCCCCCCCCCCC(=O)NCCN(CCC(=O)NCCN)CCC(=O)NCCN)nn2)c1]

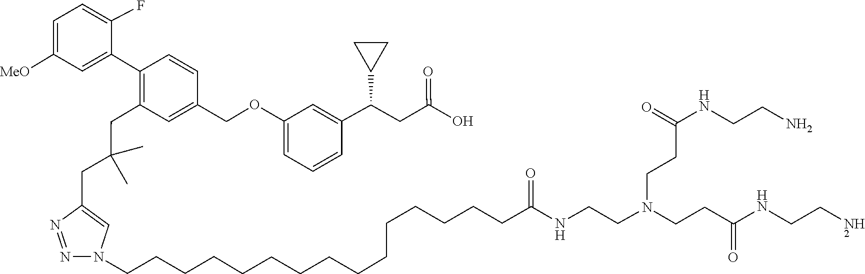 [SMILES: COc1ccc(F)c(-c2ccc(COc3cccc([C@@H](CC(=O)O)C4CC4)c3)cc2CC(C)(C)Cc2cn(CCCCCCCCCCCCCCCC(=O)NCCN(CCC(=O)NCCN)CCC(=O)NCCN)nn2)c1]

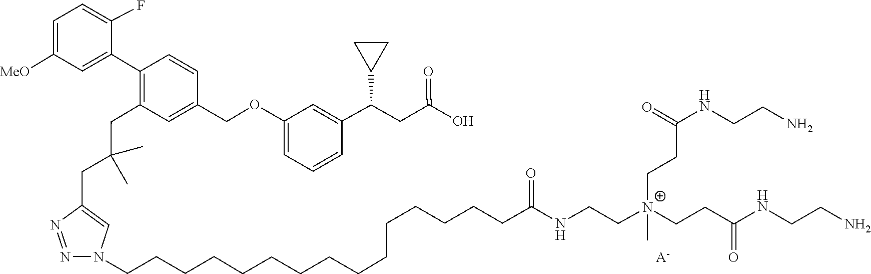

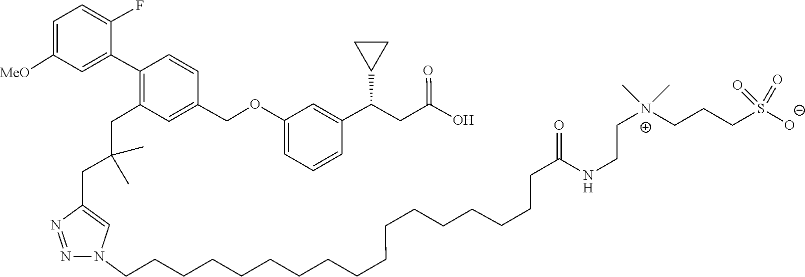 [SMILES: COc1ccc(F)c(-c2ccc(COc3cccc([C@@H](CC(=O)O)C4CC4)c3)cc2CC(C)(C)Cc2cn(CCCCCCCCCCCCCCCCCC(=O)NCC[N+](C)(C)CCCS(=O)(=O)[O-])nn2)c1]

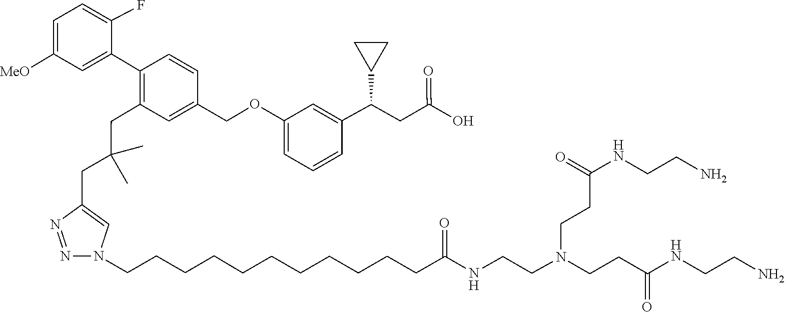 [SMILES: COc1ccc(F)c(-c2ccc(COc3cccc([C@@H](CC(=O)O)C4CC4)c3)cc2CC(C)(C)Cc2cn(CCCCCCCCCCCC(=O)NCCN(CCC(=O)NCCN)CCC(=O)NCCN)nn2)c1]

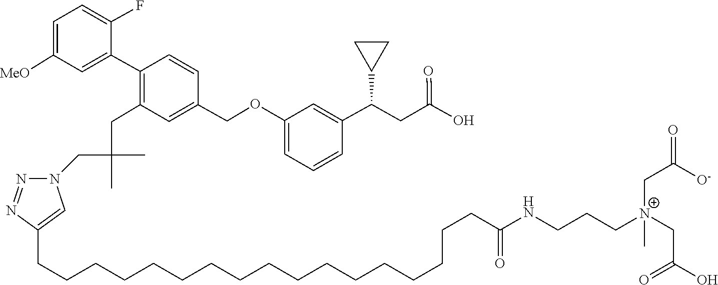 [SMILES: COc1ccc(F)c(-c2ccc(COc3cccc([C@@H](CC(=O)O)C4CC4)c3)cc2CC(C)(C)Cn2cc(CCCCCCCCCCCCCCCCCC(=O)NCCC[N+](C)(CC(=O)[O-])CC(=O)O)nn2)c1]

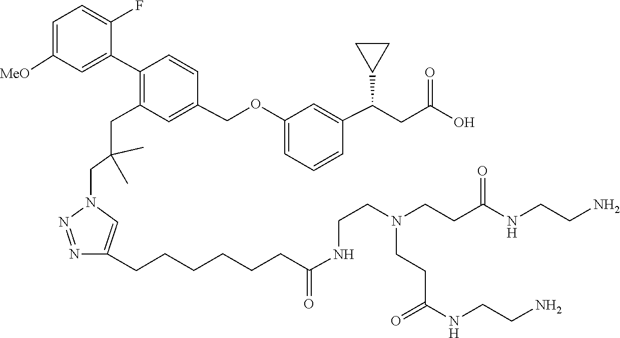 [SMILES: COc1ccc(F)c(-c2ccc(COc3cccc([C@@H](CC(=O)O)C4CC4)c3)cc2CC(C)(C)Cn2cc(CCCCCCC(=O)NCCN(CCC(=O)NCCN)CCC(=O)NCCN)nn2)c1]

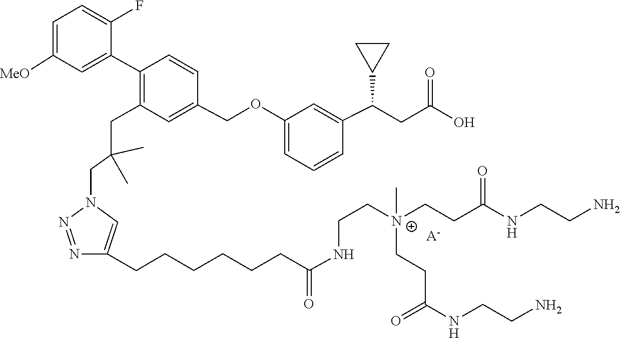

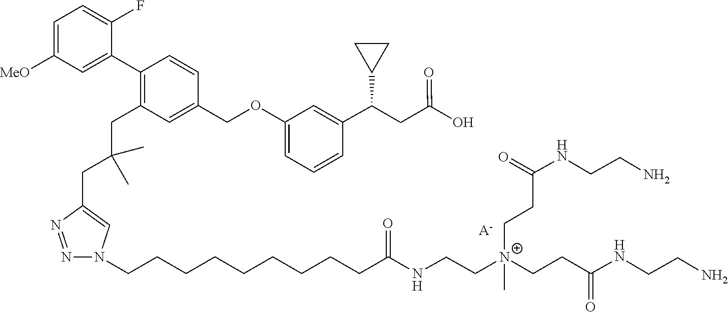

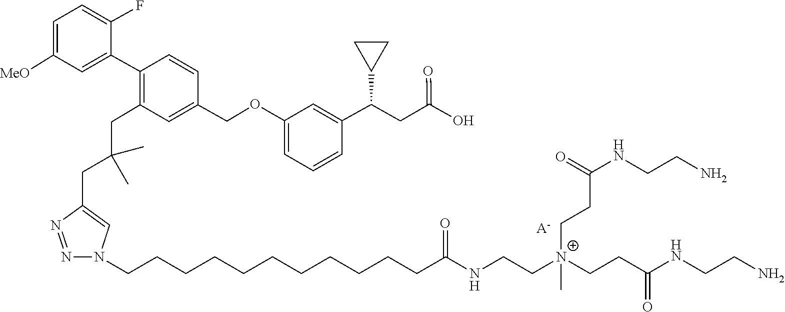

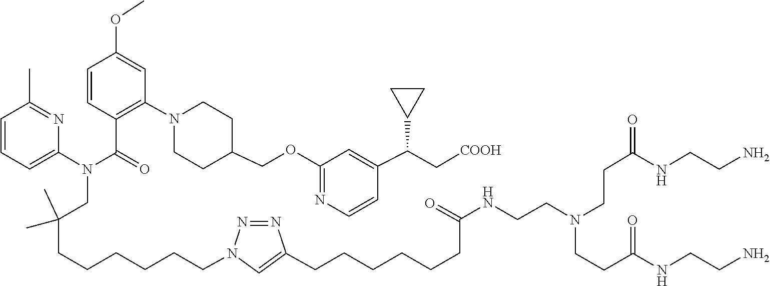 [SMILES: COc1ccc(C(=O)N(CC(C)(C)CCCCCCn2cc(CCCCCCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

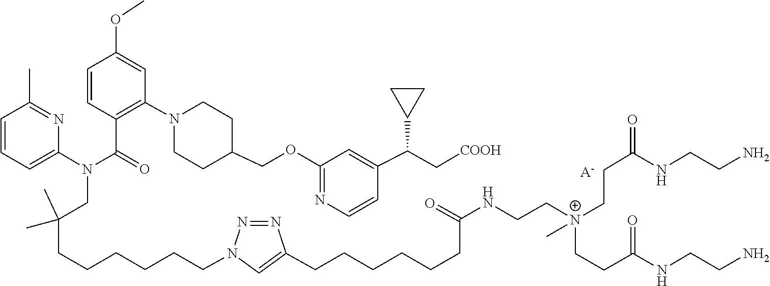

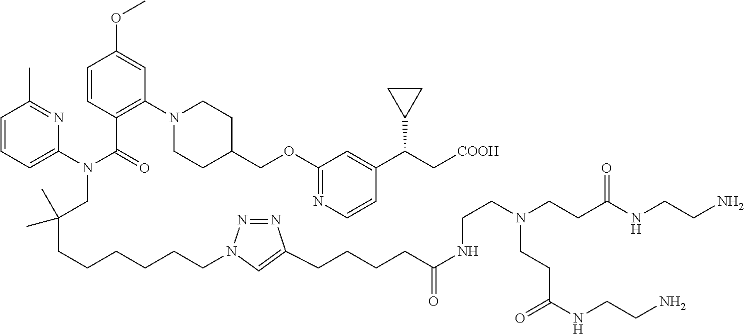 [SMILES: COc1ccc(C(=O)N(CC(C)(C)CCCCCCn2cc(CCCCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

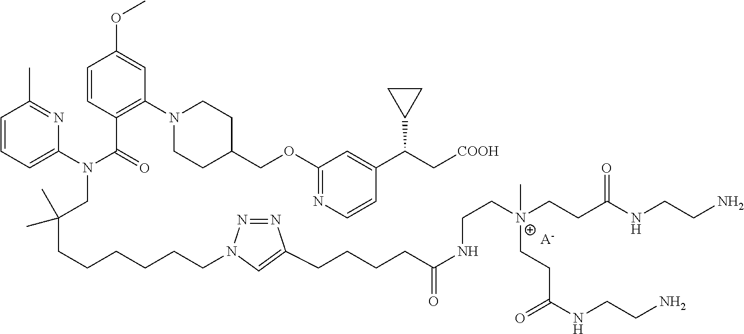

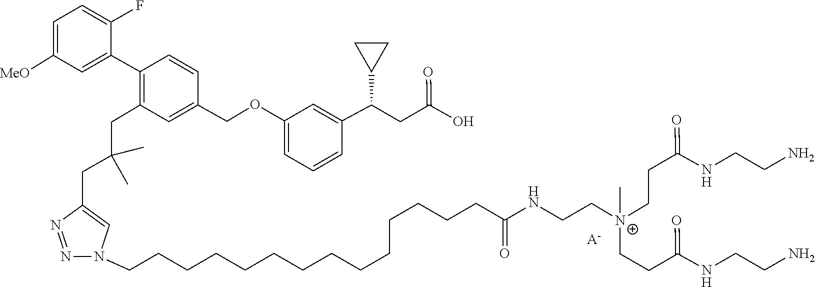

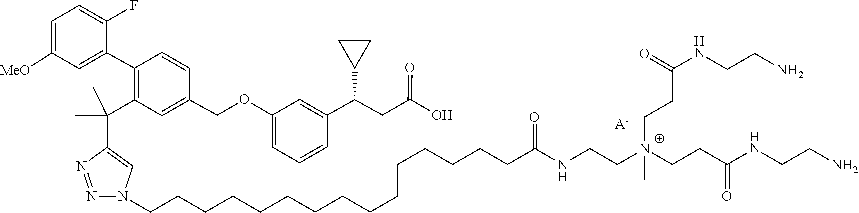

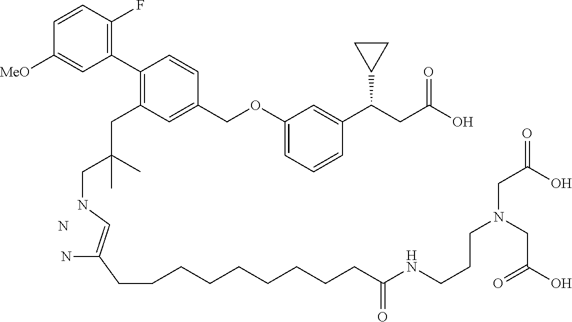

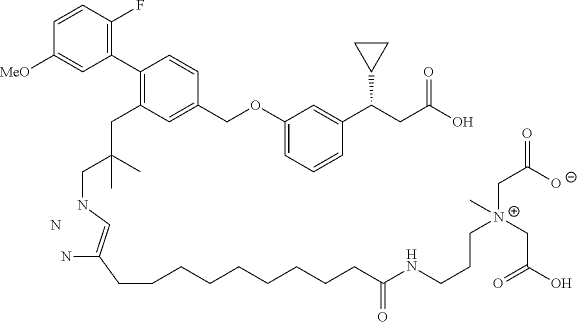

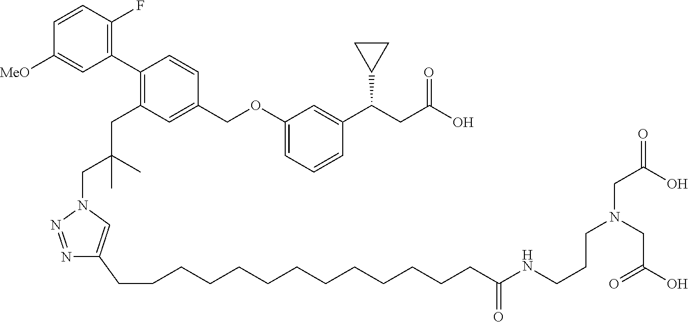 [SMILES: COc1ccc(F)c(-c2ccc(COc3cccc([C@@H](CC(=O)O)C4CC4)c3)cc2CC(C)(C)Cn2cc(CCCCCCCCCCCCCC(=O)NCCCN(CC(=O)O)CC(=O)O)nn2)c1]

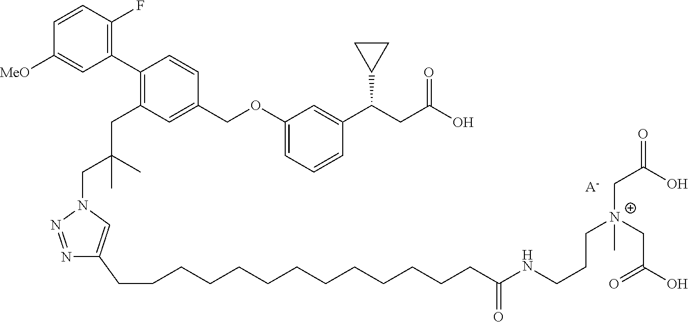

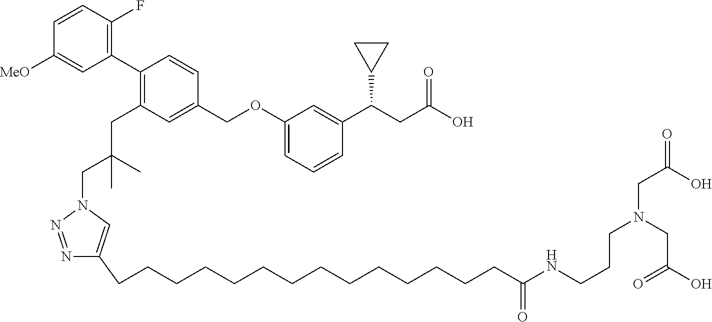 [SMILES: COc1ccc(F)c(-c2ccc(COc3cccc([C@@H](CC(=O)O)C4CC4)c3)cc2CC(C)(C)Cn2cc(CCCCCCCCCCCCCCC(=O)NCCCN(CC(=O)O)CC(=O)O)nn2)c1]

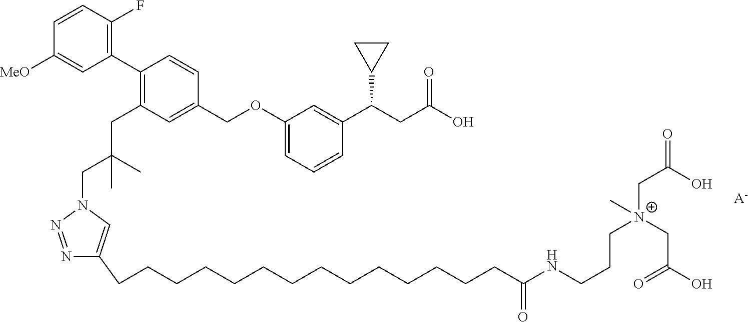

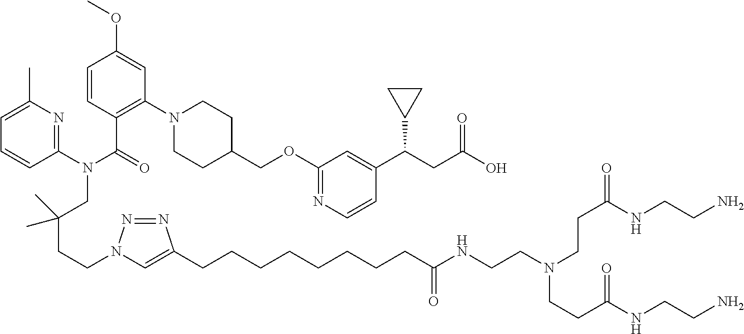 [SMILES: COc1ccc(C(=O)N(CC(C)(C)CCn2cc(CCCCCCCCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

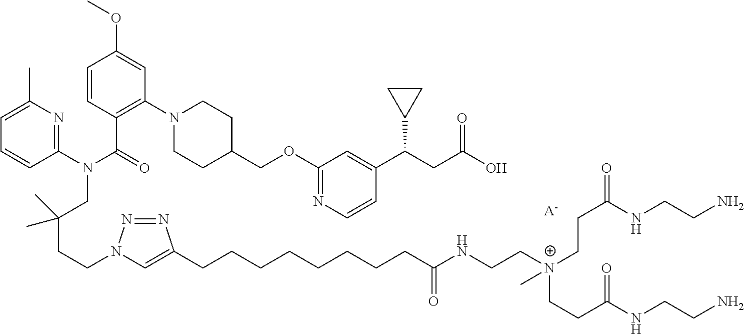

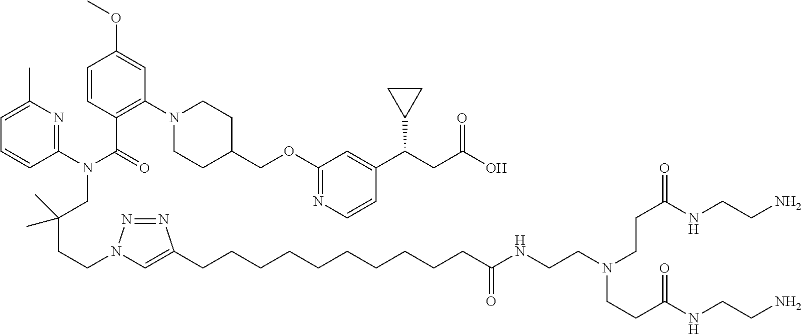 [SMILES: COc1ccc(C(=O)N(CC(C)(C)CCn2cc(CCCCCCCCCCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

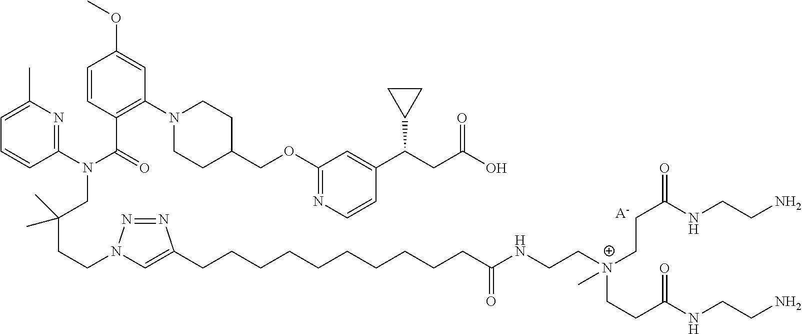

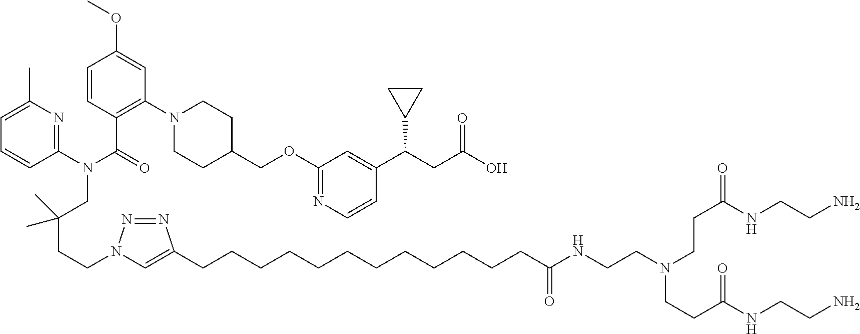 [SMILES: COc1ccc(C(=O)N(CC(C)(C)CCn2cc(CCCCCCCCCCCCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

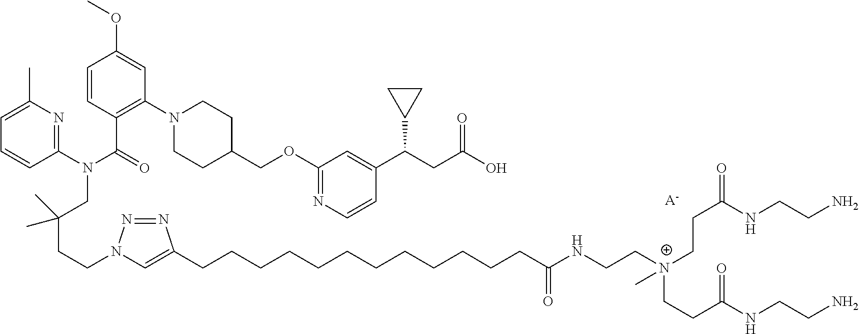

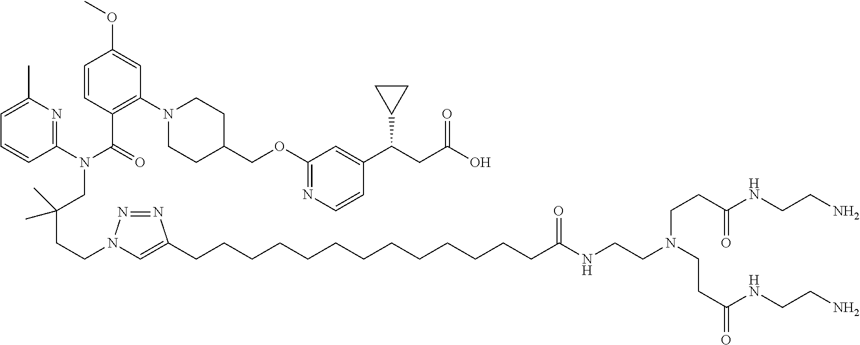 [SMILES: COc1ccc(C(=O)N(CC(C)(C)CCn2cc(CCCCCCCCCCCCCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

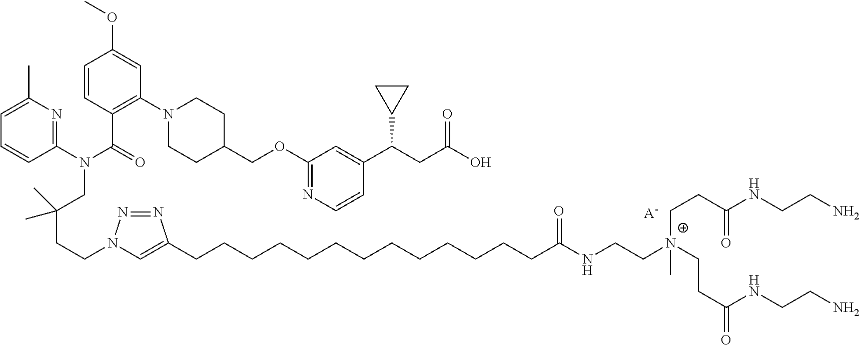

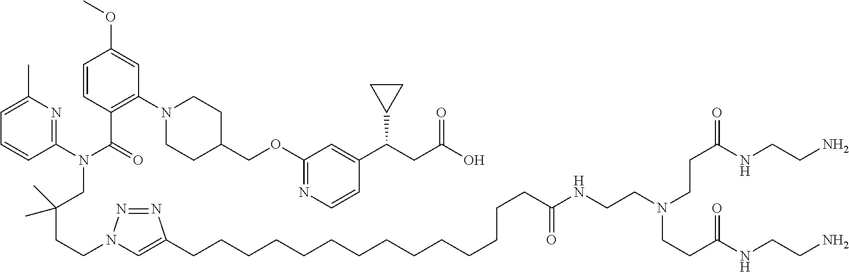 [SMILES: COc1ccc(C(=O)N(CC(C)(C)CCn2cc(CCCCCCCCCCCCCCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

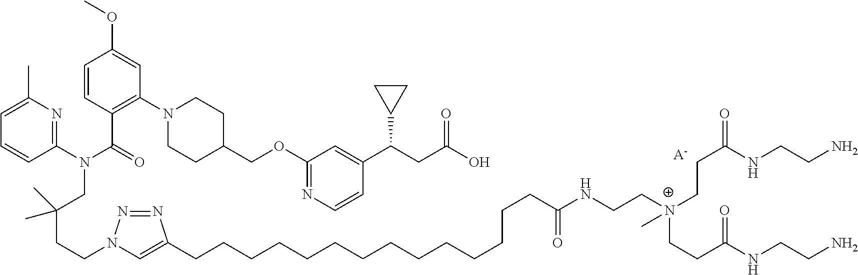

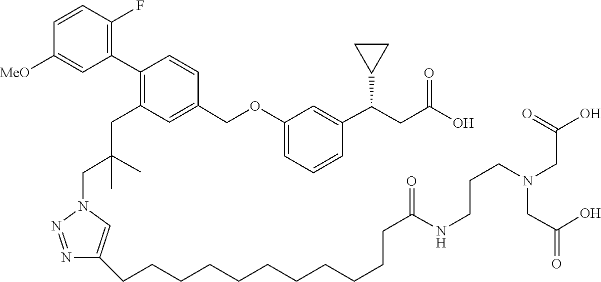 [SMILES: COc1ccc(F)c(-c2ccc(COc3cccc([C@@H](CC(=O)O)C4CC4)c3)cc2CC(C)(C)Cn2cc(CCCCCCCCCCCC(=O)NCCCN(CC(=O)O)CC(=O)O)nn2)c1]

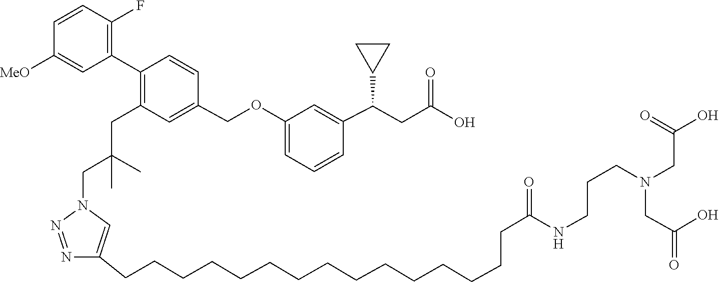 [SMILES: COc1ccc(F)c(-c2ccc(COc3cccc([C@@H](CC(=O)O)C4CC4)c3)cc2CC(C)(C)Cn2cc(CCCCCCCCCCCCCCCC(=O)NCCCN(CC(=O)O)CC(=O)O)nn2)c1]

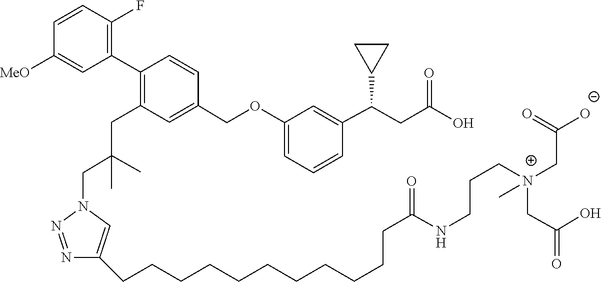 [SMILES: COc1ccc(F)c(-c2ccc(COc3cccc([C@@H](CC(=O)O)C4CC4)c3)cc2CC(C)(C)Cn2cc(CCCCCCCCCCCC(=O)NCCC[N+](C)(CC(=O)[O-])CC(=O)O)nn2)c1]

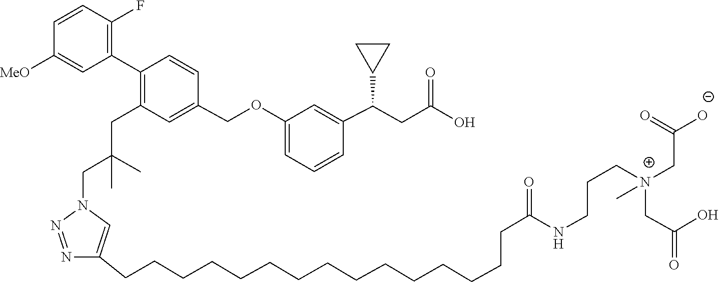 [SMILES: COc1ccc(F)c(-c2ccc(COc3cccc([C@@H](CC(=O)O)C4CC4)c3)cc2CC(C)(C)Cn2cc(CCCCCCCCCCCCCCCC(=O)NCCC[N+](C)(CC(=O)[O-])CC(=O)O)nn2)c1]

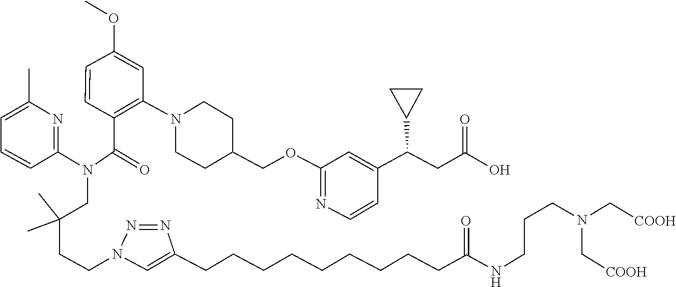 [SMILES: COc1ccc(C(=O)N(CC(C)(C)CCn2cc(CCCCCCCCCC(=O)NCCCN(CC(=O)O)CC(=O)O)nn2)c2cccc(C)n2)c(N2CCC(COc3cc([C@@H](CC(=O)O)C4CC4)ccn3)CC2)c1]

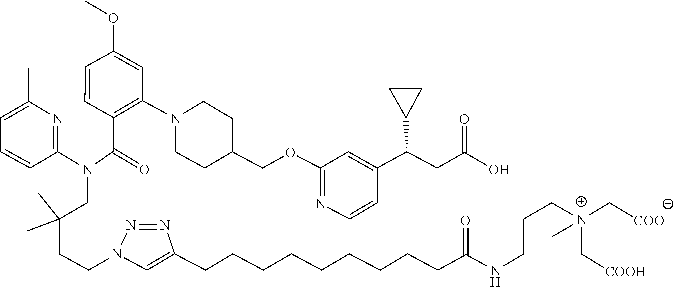

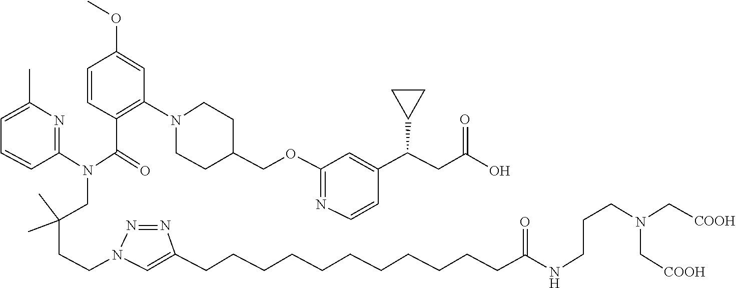 [SMILES: COc1ccc(C(=O)N(CC(C)(C)CCn2cc(CCCCCCCCCCCC(=O)NCCCN(CC(=O)O)CC(=O)O)nn2)c2cccc(C)n2)c(N2CCC(COc3cc([C@@H](CC(=O)O)C4CC4)ccn3)CC2)c1]

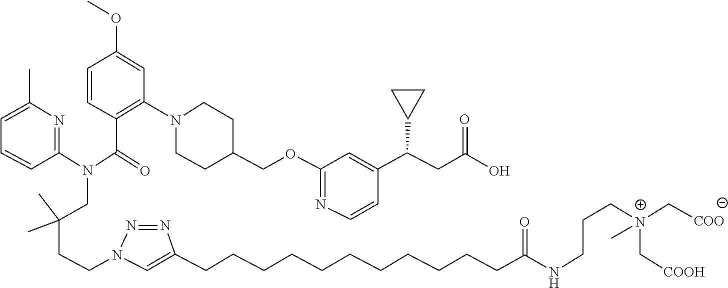

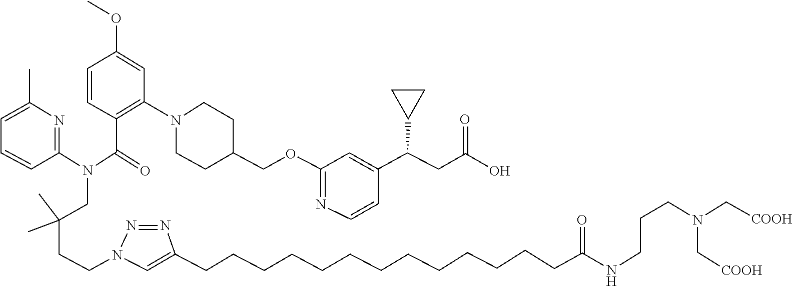 [SMILES: COc1ccc(C(=O)N(CC(C)(C)CCn2cc(CCCCCCCCCCCCCC(=O)NCCCN(CC(=O)O)CC(=O)O)nn2)c2cccc(C)n2)c(N2CCC(COc3cc([C@@H](CC(=O)O)C4CC4)ccn3)CC2)c1]

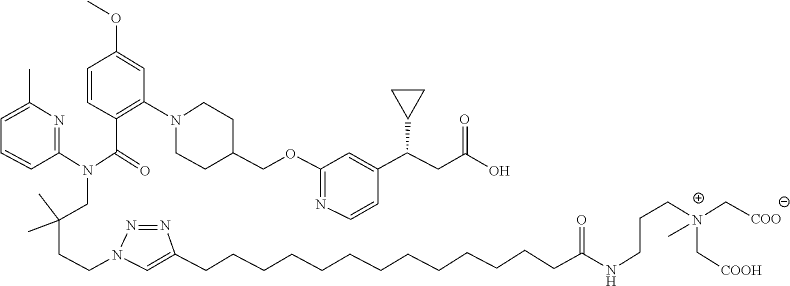

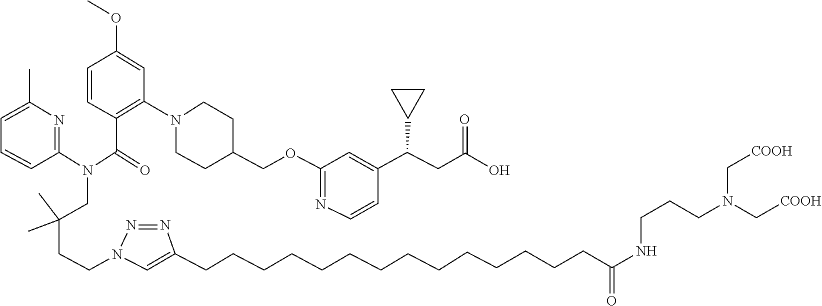 [SMILES: COc1ccc(C(=O)N(CC(C)(C)CCn2cc(CCCCCCCCCCCCCCC(=O)NCCCN(CC(=O)O)CC(=O)O)nn2)c2cccc(C)n2)c(N2CCC(COc3cc([C@@H](CC(=O)O)C4CC4)ccn3)CC2)c1]

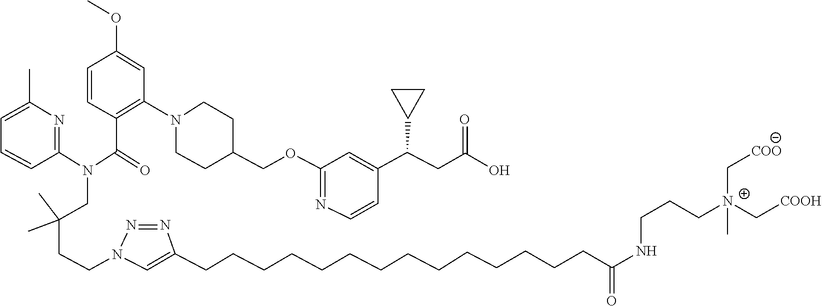

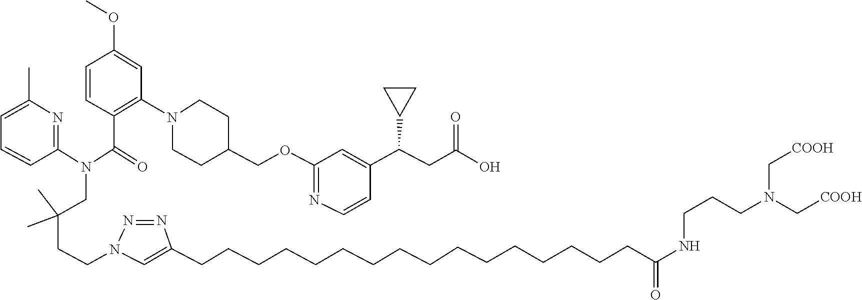 [SMILES: COc1ccc(C(=O)N(CC(C)(C)CCn2cc(CCCCCCCCCCCCCCCCC(=O)NCCCN(CC(=O)O)CC(=O)O)nn2)c2cccc(C)n2)c(N2CCC(COc3cc([C@@H](CC(=O)O)C4CC4)ccn3)CC2)c1]

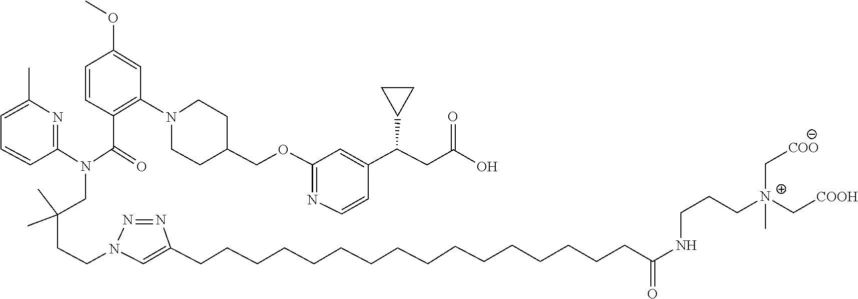

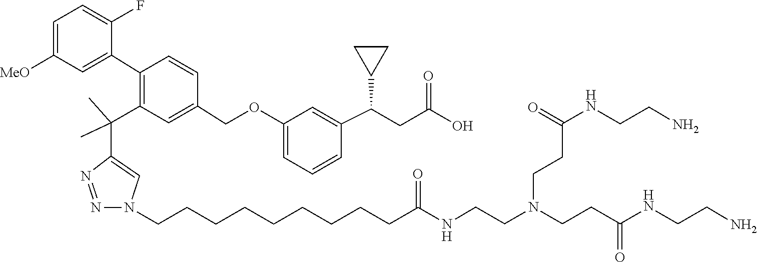 [SMILES: COc1ccc(F)c(-c2ccc(COc3cccc([C@@H](CC(=O)O)C4CC4)c3)cc2C(C)(C)c2cn(CCCCCCCCCC(=O)NCCN(CCC(=O)NCCN)CCC(=O)NCCN)nn2)c1]

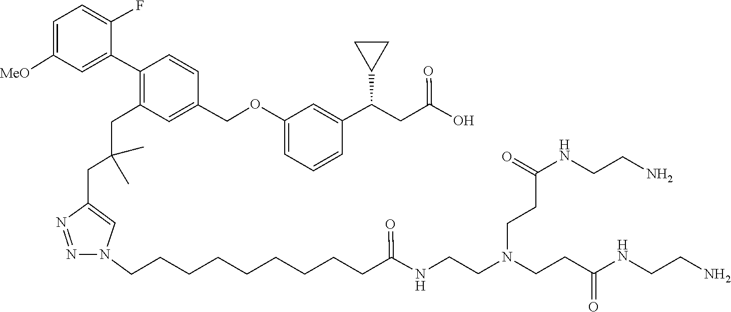 [SMILES: COc1ccc(F)c(-c2ccc(COc3cccc([C@@H](CC(=O)O)C4CC4)c3)cc2CC(C)(C)Cc2cn(CCCCCCCCCC(=O)NCCN(CCC(=O)NCCN)CCC(=O)NCCN)nn2)c1]

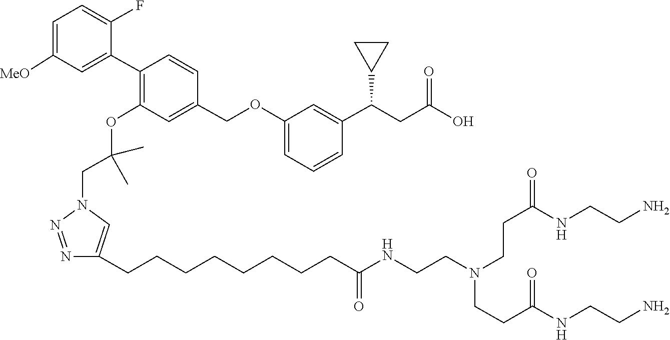 [SMILES: COc1ccc(F)c(-c2ccc(COc3cccc([C@@H](CC(=O)O)C4CC4)c3)cc2OC(C)(C)Cn2cc(CCCCCCCCC(=O)NCCN(CCC(=O)NCCN)CCC(=O)NCCN)nn2)c1]

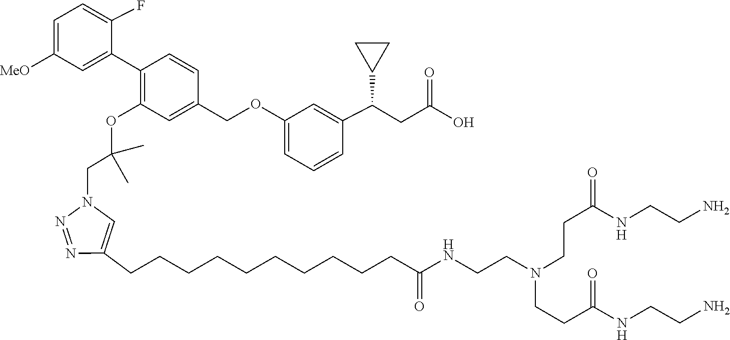 [SMILES: COc1ccc(F)c(-c2ccc(COc3cccc([C@@H](CC(=O)O)C4CC4)c3)cc2OC(C)(C)Cn2cc(CCCCCCCCCCC(=O)NCCN(CCC(=O)NCCN)CCC(=O)NCCN)nn2)c1]

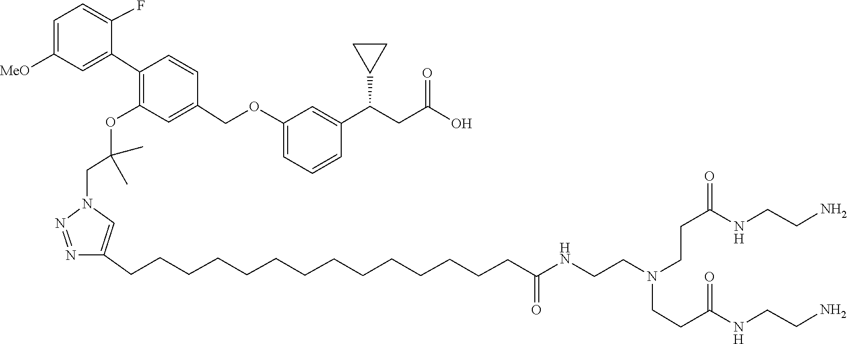 [SMILES: COc1ccc(F)c(-c2ccc(COc3cccc([C@@H](CC(=O)O)C4CC4)c3)cc2OC(C)(C)Cn2cc(CCCCCCCCCCCCCCC(=O)NCCN(CCC(=O)NCCN)CCC(=O)NCCN)nn2)c1]

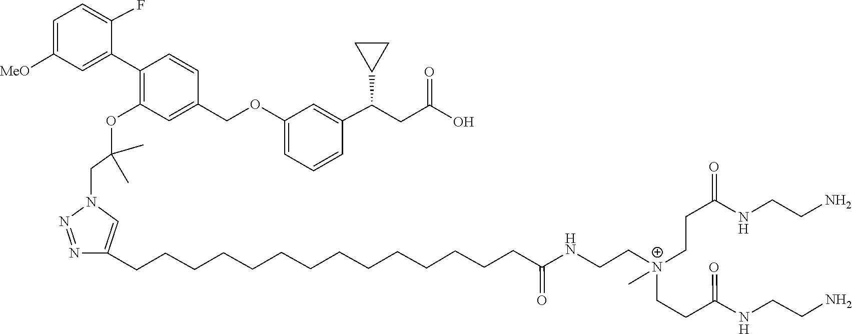 [SMILES: COc1ccc(F)c(-c2ccc(COc3cccc([C@@H](CC(=O)O)C4CC4)c3)cc2OC(C)(C)Cn2cc(CCCCCCCCCCCCCCC(=O)NCC[N+](C)(CCC(=O)NCCN)CCC(=O)NCCN)nn2)c1]

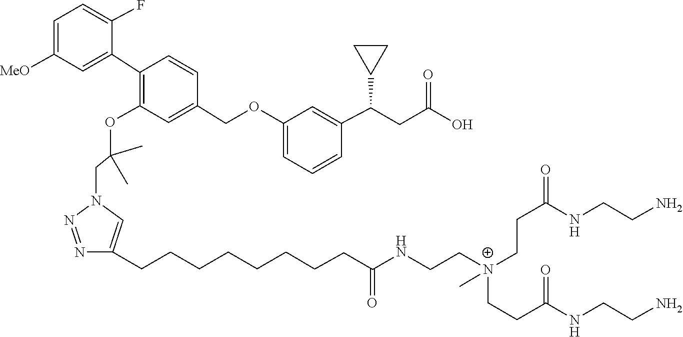 [SMILES: COc1ccc(F)c(-c2ccc(COc3cccc([C@@H](CC(=O)O)C4CC4)c3)cc2OC(C)(C)Cn2cc(CCCCCCCCC(=O)NCC[N+](C)(CCC(=O)NCCN)CCC(=O)NCCN)nn2)c1]

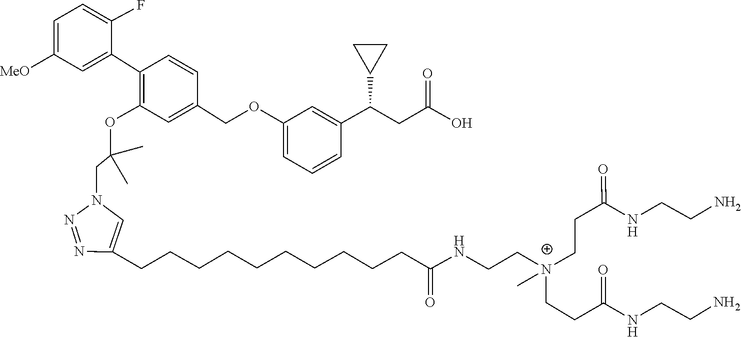 [SMILES: COc1ccc(F)c(-c2ccc(COc3cccc([C@@H](CC(=O)O)C4CC4)c3)cc2OC(C)(C)Cn2cc(CCCCCCCCCCC(=O)NCC[N+](C)(CCC(=O)NCCN)CCC(=O)NCCN)nn2)c1]

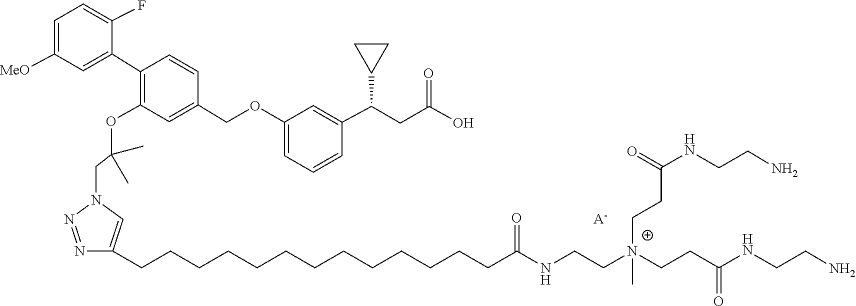

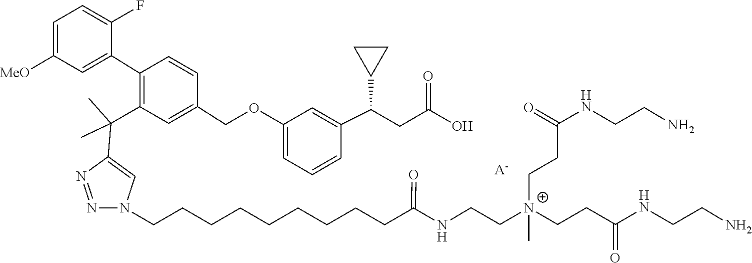

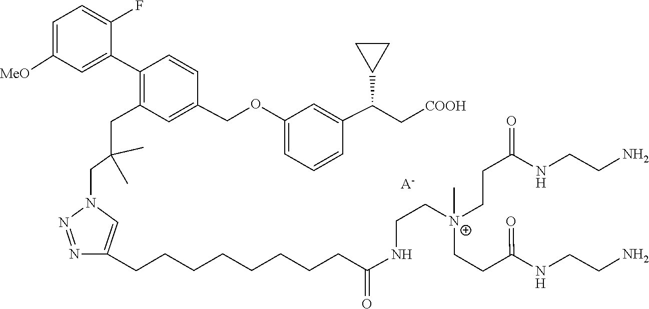

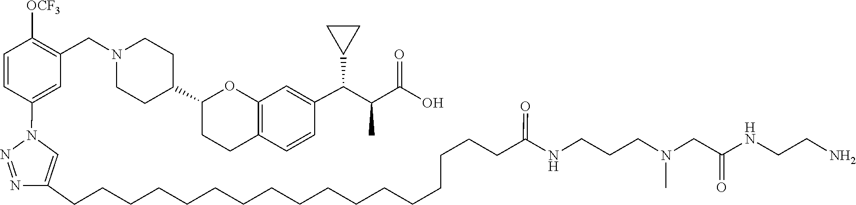 [SMILES: C[C@H](C(=O)O)[C@H](c1ccc2c(c1)O[C@@H](C1CCN(Cc3cc(-n4cc(CCCCCCCCCCCCCCCCCC(=O)NCCCN(C)CC(=O)NCCN)nn4)ccc3OC(F)(F)F)CC1)CC2)C1CC1]

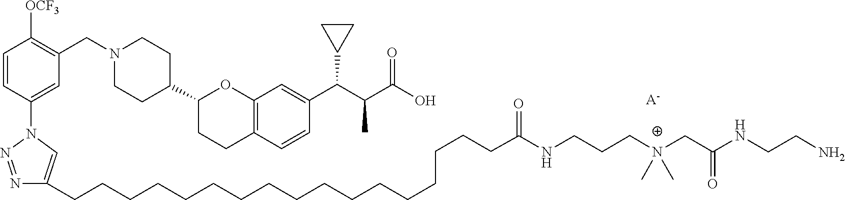

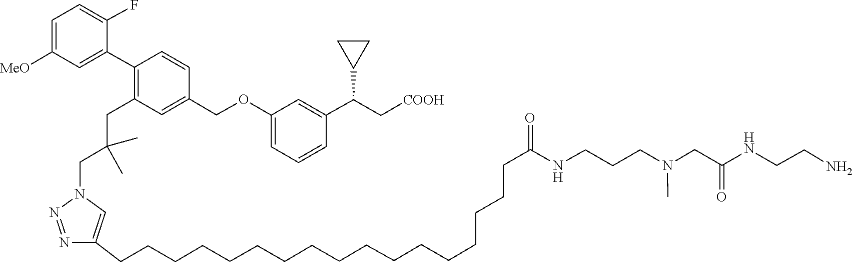 [SMILES: COc1ccc(F)c(-c2ccc(COc3cccc([C@@H](CC(=O)O)C4CC4)c3)cc2CC(C)(C)Cn2cc(CCCCCCCCCCCCCCCCCC(=O)NCCCN(C)CC(=O)NCCN)nn2)c1]

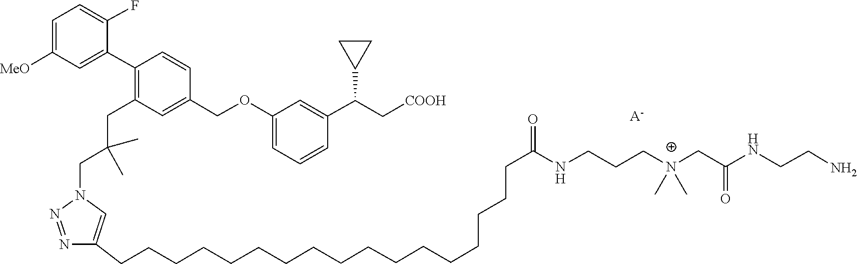

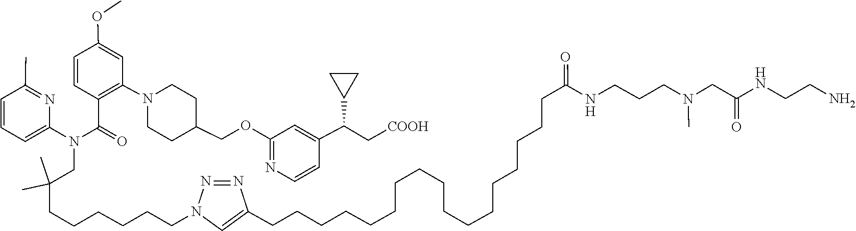 [SMILES: COc1ccc(C(=O)N(CC(C)(C)CCCCCCn2cc(CCCCCCCCCCCCCCCCCC(=O)NCCCN(C)CC(=O)NCCN)nn2)c2cccc(C)n2)c(N2CCC(COc3cc([C@@H](CC(=O)O)C4CC4)ccn3)CC2)c1]

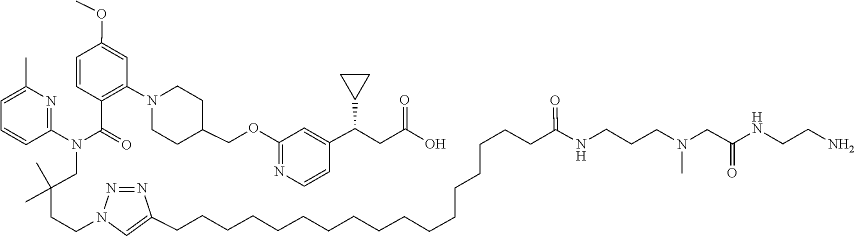 [SMILES: COc1ccc(C(=O)N(CC(C)(C)CCn2cc(CCCCCCCCCCCCCCCCCC(=O)NCCCN(C)CC(=O)NCCN)nn2)c2cccc(C)n2)c(N2CCC(COc3cc([C@@H](CC(=O)O)C4CC4)ccn3)CC2)c1]

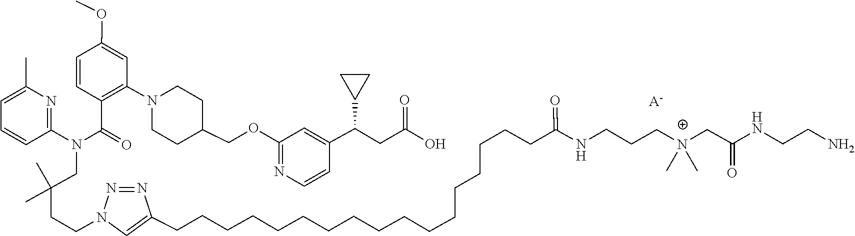

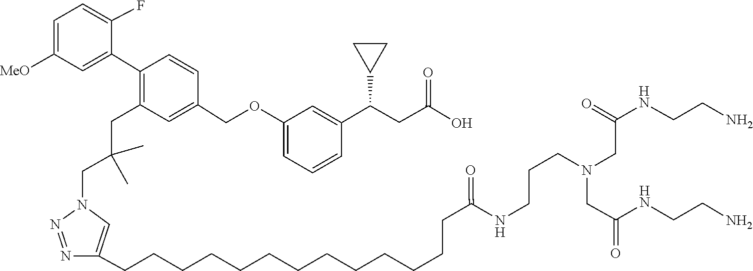 [SMILES: COc1ccc(F)c(-c2ccc(COc3cccc([C@@H](CC(=O)O)C4CC4)c3)cc2CC(C)(C)Cn2cc(CCCCCCCCCCCCCC(=O)NCCCN(CC(=O)NCCN)CC(=O)NCCN)nn2)c1]

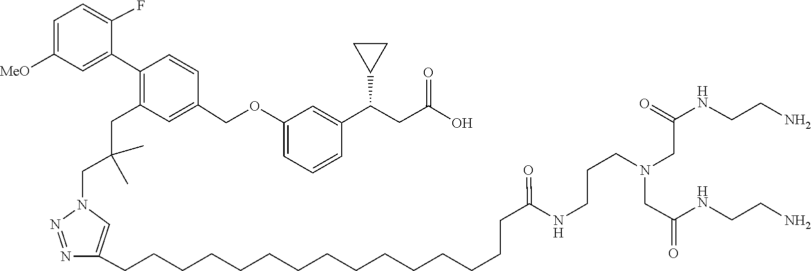 [SMILES: COc1ccc(F)c(-c2ccc(COc3cccc([C@@H](CC(=O)O)C4CC4)c3)cc2CC(C)(C)Cn2cc(CCCCCCCCCCCCCCCC(=O)NCCCN(CC(=O)NCCN)CC(=O)NCCN)nn2)c1]

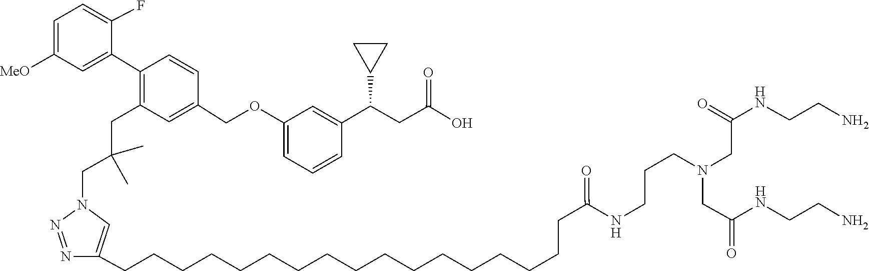 [SMILES: COc1ccc(F)c(-c2ccc(COc3cccc([C@@H](CC(=O)O)C4CC4)c3)cc2CC(C)(C)Cn2cc(CCCCCCCCCCCCCCCCCC(=O)NCCCN(CC(=O)NCCN)CC(=O)NCCN)nn2)c1]

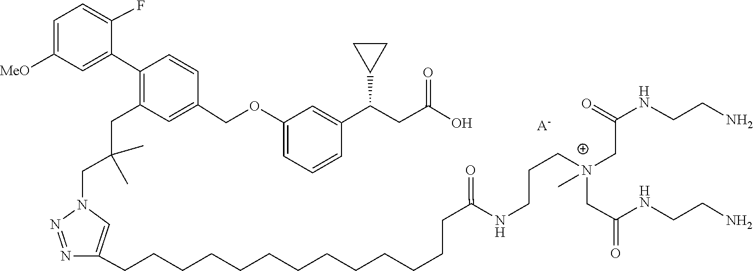

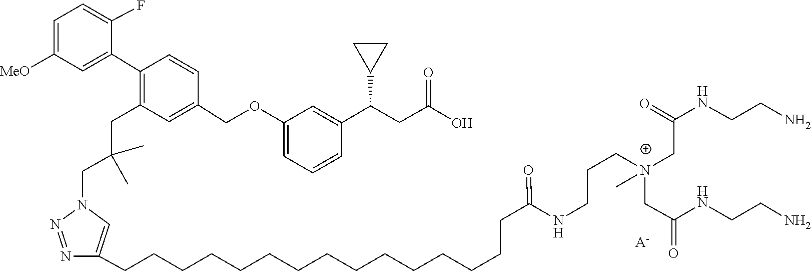

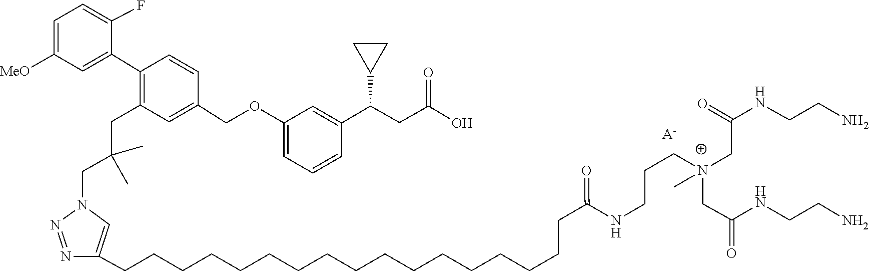

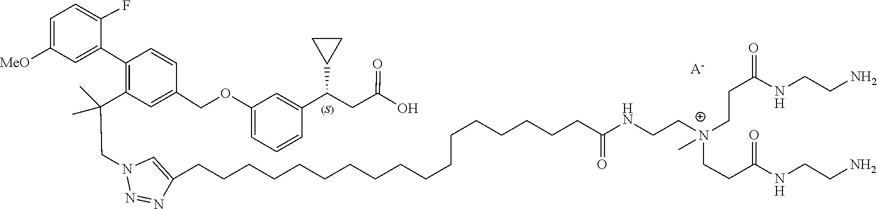

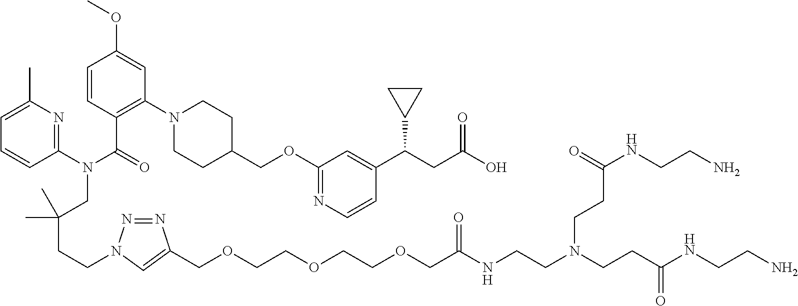 [SMILES: COc1ccc(C(=O)N(CC(C)(C)CCn2cc(COCCOCCOCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

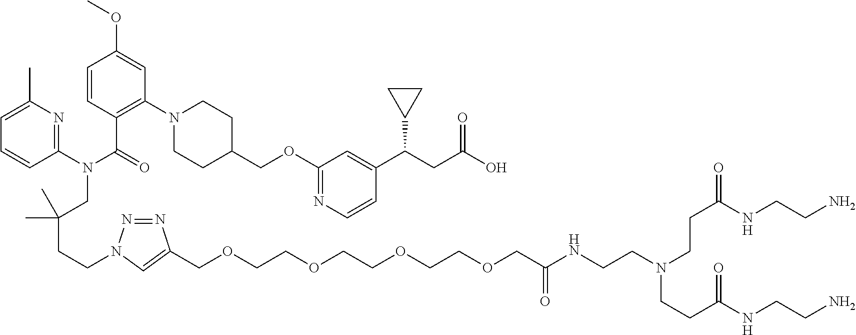 [SMILES: COc1ccc(C(=O)N(CC(C)(C)CCn2cc(COCCOCCOCCOCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

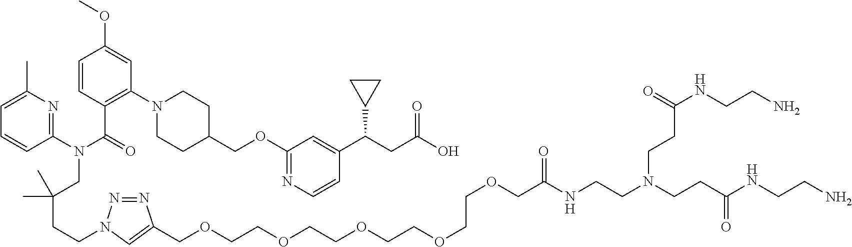 [SMILES: COc1ccc(C(=O)N(CC(C)(C)CCn2cc(COCCOCCOCCOCCOCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

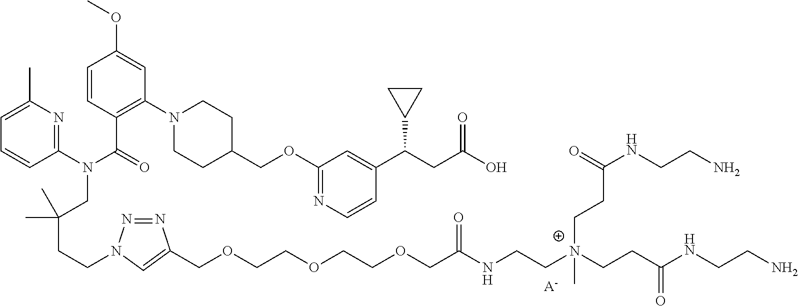

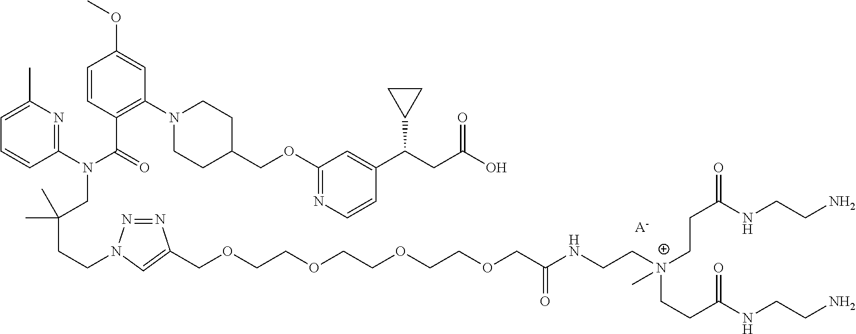

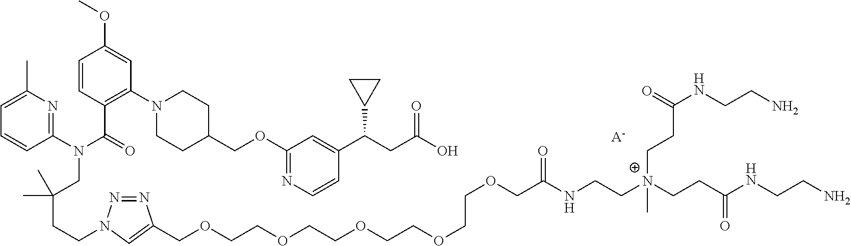

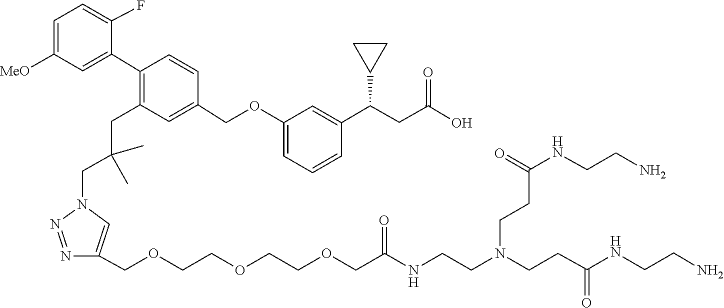 [SMILES: COc1ccc(F)c(-c2ccc(COc3cccc([C@@H](CC(=O)O)C4CC4)c3)cc2CC(C)(C)Cn2cc(COCCOCCOCC(=O)NCCN(CCC(=O)NCCN)CCC(=O)NCCN)nn2)c1]

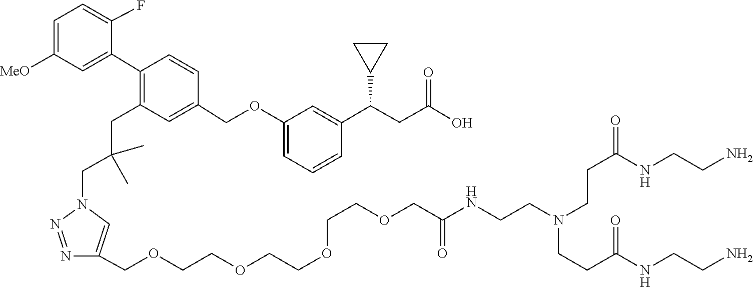 [SMILES: COc1ccc(F)c(-c2ccc(COc3cccc([C@@H](CC(=O)O)C4CC4)c3)cc2CC(C)(C)Cn2cc(COCCOCCOCCOCC(=O)NCCN(CCC(=O)NCCN)CCC(=O)NCCN)nn2)c1]

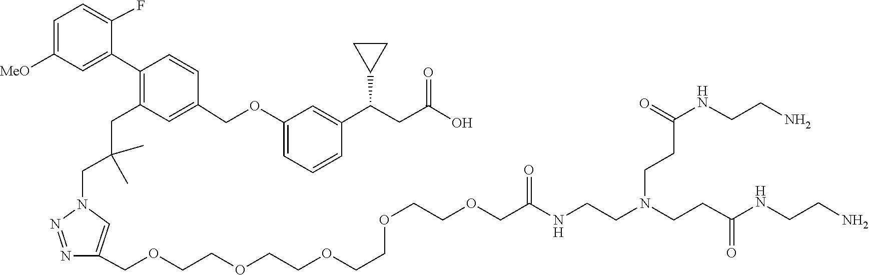 [SMILES: COc1ccc(F)c(-c2ccc(COc3cccc([C@@H](CC(=O)O)C4CC4)c3)cc2CC(C)(C)Cn2cc(COCCOCCOCCOCCOCC(=O)NCCN(CCC(=O)NCCN)CCC(=O)NCCN)nn2)c1]

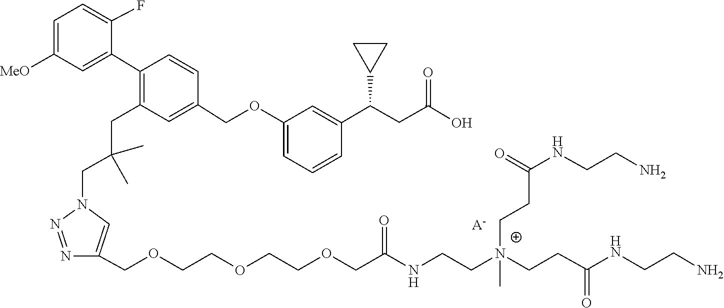

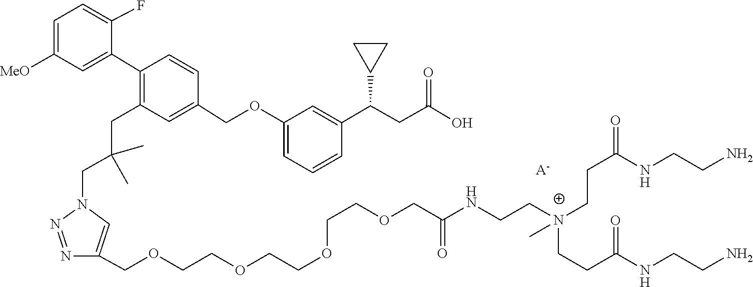

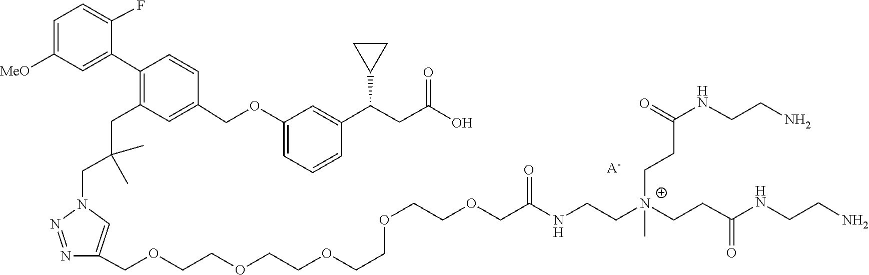

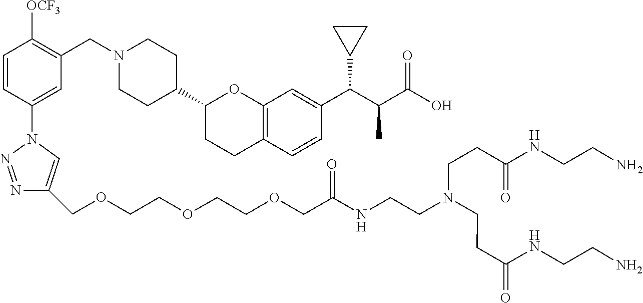 [SMILES: C[C@H](C(=O)O)[C@H](c1ccc2c(c1)O[C@@H](C1CCN(Cc3cc(-n4cc(COCCOCCOCC(=O)NCCN(CCC(=O)NCCN)CCC(=O)NCCN)nn4)ccc3OC(F)(F)F)CC1)CC2)C1CC1]

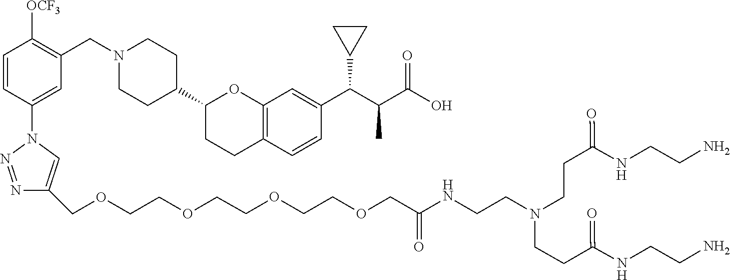 [SMILES: C[C@H](C(=O)O)[C@H](c1ccc2c(c1)O[C@@H](C1CCN(Cc3cc(-n4cc(COCCOCCOCCOCC(=O)NCCN(CCC(=O)NCCN)CCC(=O)NCCN)nn4)ccc3OC(F)(F)F)CC1)CC2)C1CC1]

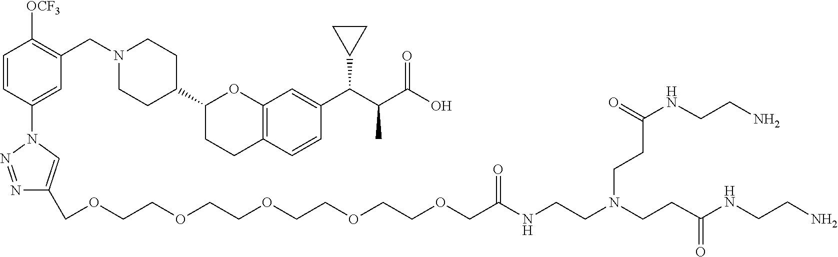 [SMILES: C[C@H](C(=O)O)[C@H](c1ccc2c(c1)O[C@@H](C1CCN(Cc3cc(-n4cc(COCCOCCOCCOCCOCC(=O)NCCN(CCC(=O)NCCN)CCC(=O)NCCN)nn4)ccc3OC(F)(F)F)CC1)CC2)C1CC1]

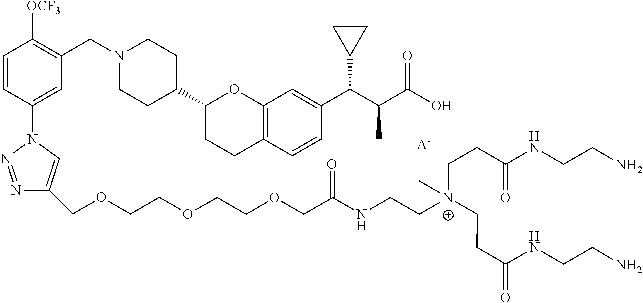

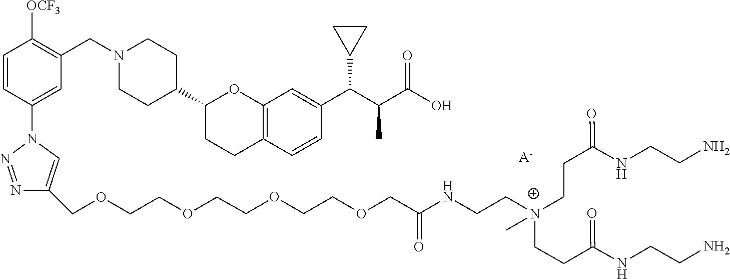

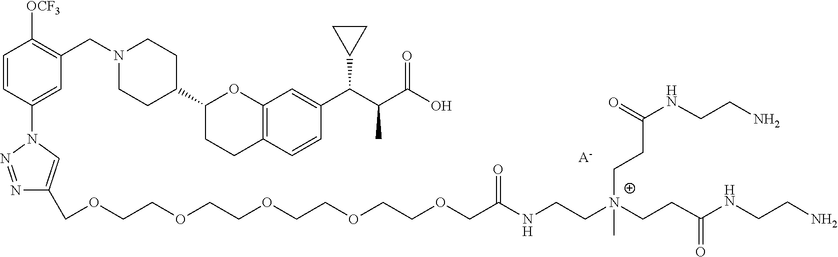

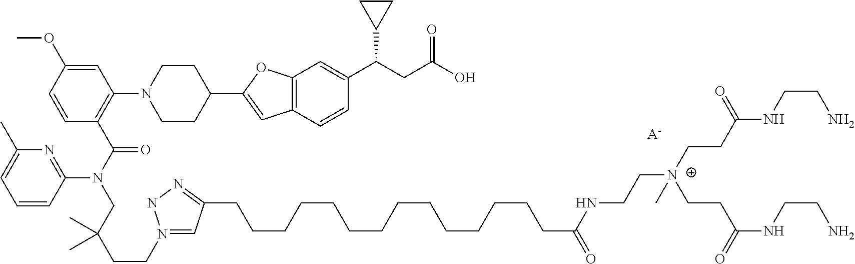

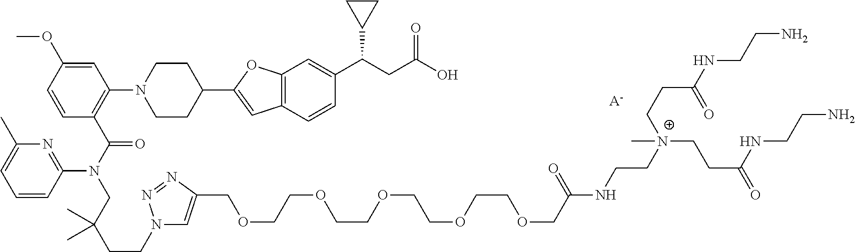

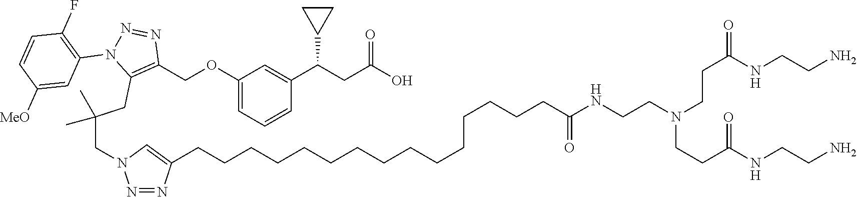 [SMILES: COc1ccc(F)c(-n2nnc(COc3cccc([C@@H](CC(=O)O)C4CC4)c3)c2CC(C)(C)Cn2cc(CCCCCCCCCCCCCCCC(=O)NCCN(CCC(=O)NCCN)CCC(=O)NCCN)nn2)c1]

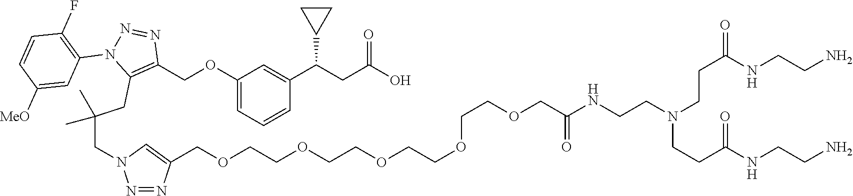 [SMILES: COc1ccc(F)c(-n2nnc(COc3cccc([C@@H](CC(=O)O)C4CC4)c3)c2CC(C)(C)Cn2cc(COCCOCCOCCOCCOCC(=O)NCCN(CCC(=O)NCCN)CCC(=O)NCCN)nn2)c1]

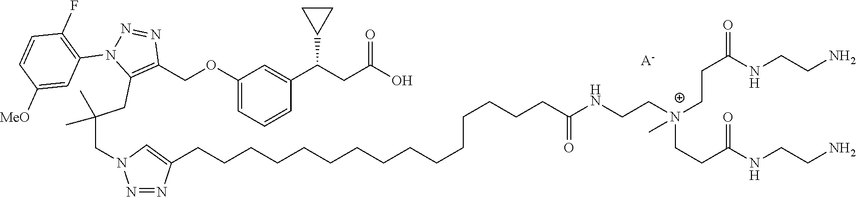

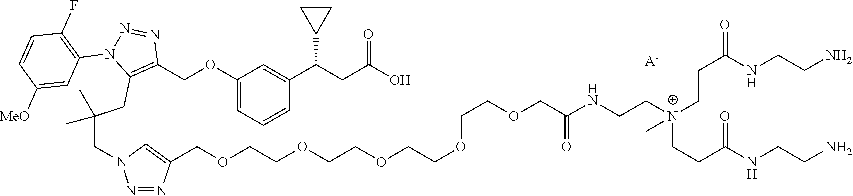

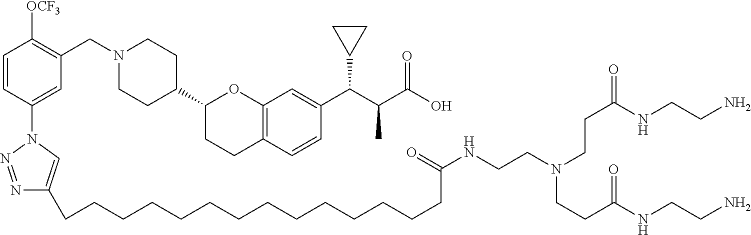 [SMILES: C[C@H](C(=O)O)[C@H](c1ccc2c(c1)O[C@@H](C1CCN(Cc3cc(-n4cc(CCCCCCCCCCCCCCC(=O)NCCN(CCC(=O)NCCN)CCC(=O)NCCN)nn4)ccc3OC(F)(F)F)CC1)CC2)C1CC1]

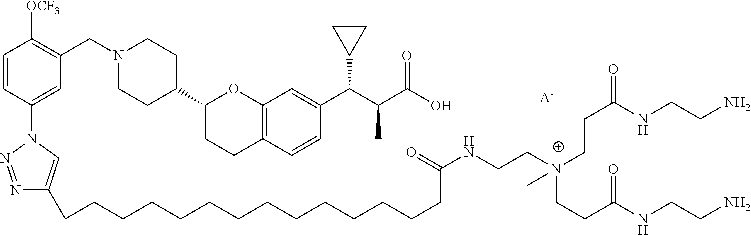

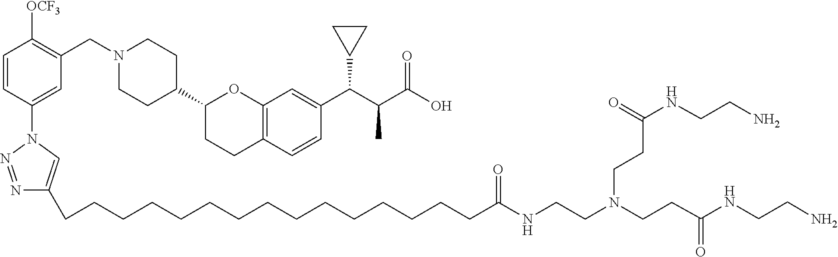 [SMILES: C[C@H](C(=O)O)[C@H](c1ccc2c(c1)O[C@@H](C1CCN(Cc3cc(-n4cc(CCCCCCCCCCCCCCCC(=O)NCCN(CCC(=O)NCCN)CCC(=O)NCCN)nn4)ccc3OC(F)(F)F)CC1)CC2)C1CC1]

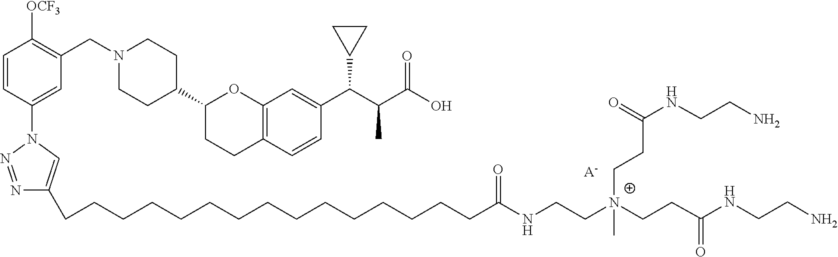

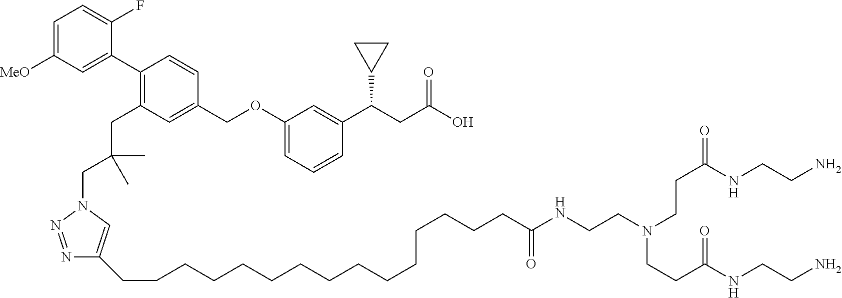 [SMILES: COc1ccc(F)c(-c2ccc(COc3cccc([C@@H](CC(=O)O)C4CC4)c3)cc2CC(C)(C)Cn2cc(CCCCCCCCCCCCCCCC(=O)NCCN(CCC(=O)NCCN)CCC(=O)NCCN)nn2)c1]

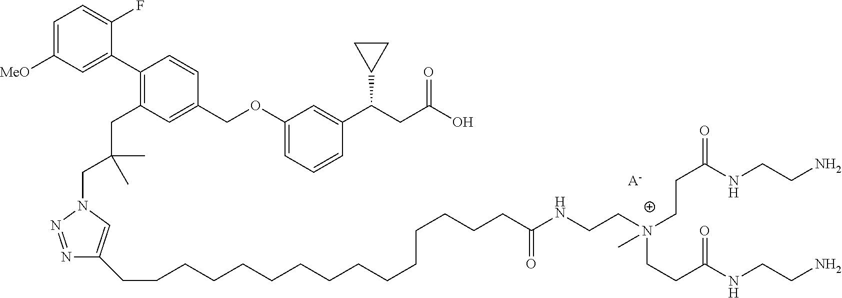

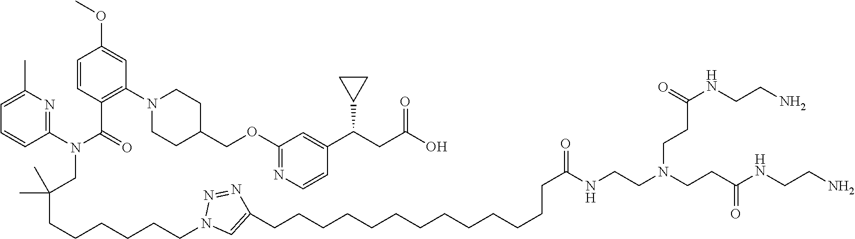 [SMILES: COc1ccc(C(=O)N(CC(C)(C)CCCCCCn2cc(CCCCCCCCCCCCCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

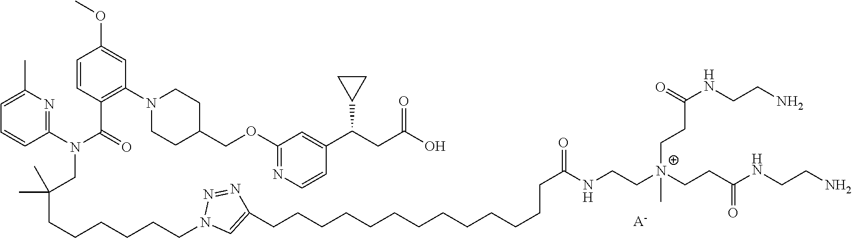

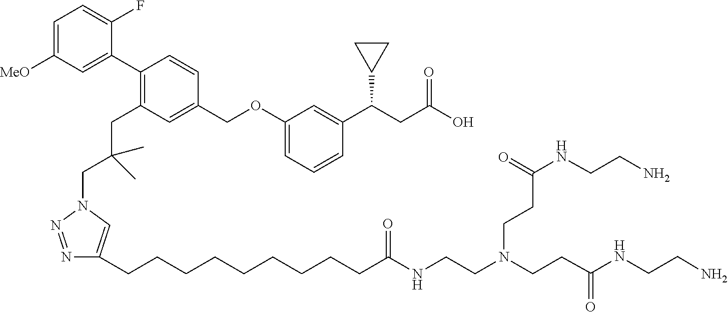 [SMILES: COc1ccc(F)c(-c2ccc(COc3cccc([C@@H](CC(=O)O)C4CC4)c3)cc2CC(C)(C)Cn2cc(CCCCCCCCCC(=O)NCCN(CCC(=O)NCCN)CCC(=O)NCCN)nn2)c1]

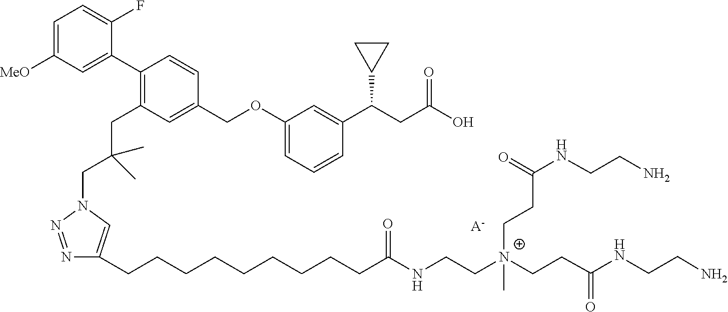

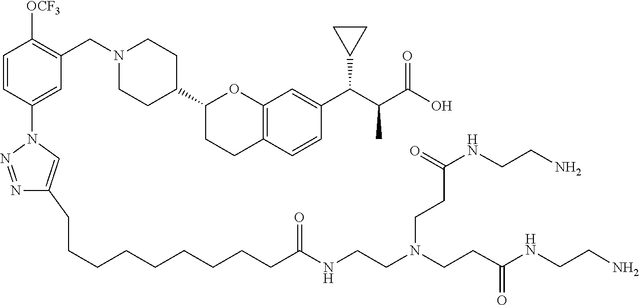 [SMILES: C[C@H](C(=O)O)[C@H](c1ccc2c(c1)O[C@@H](C1CCN(Cc3cc(-n4cc(CCCCCCCCCC(=O)NCCN(CCC(=O)NCCN)CCC(=O)NCCN)nn4)ccc3OC(F)(F)F)CC1)CC2)C1CC1]

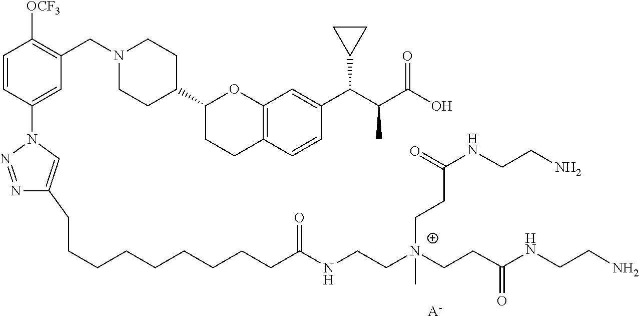

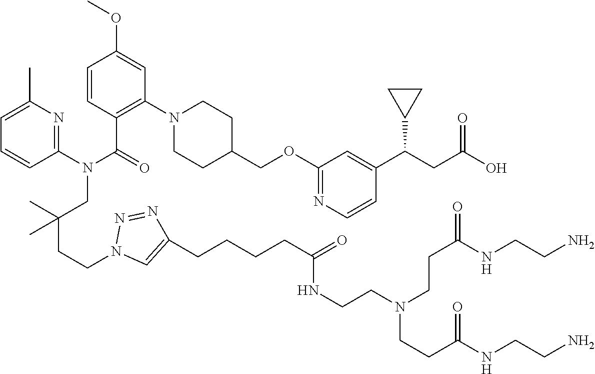 [SMILES: COc1ccc(C(=O)N(CC(C)(C)CCn2cc(CCCCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

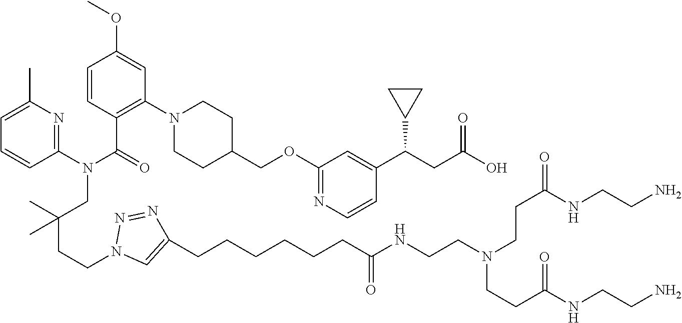 [SMILES: COc1ccc(C(=O)N(CC(C)(C)CCn2cc(CCCCCCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

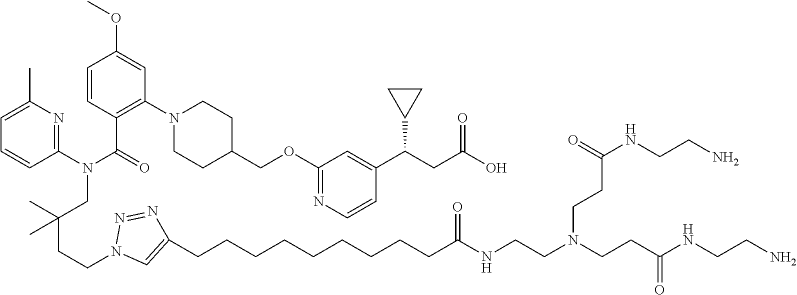 [SMILES: COc1ccc(C(=O)N(CC(C)(C)CCn2cc(CCCCCCCCCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

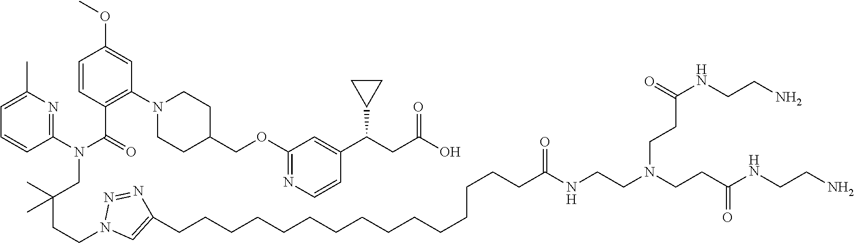 [SMILES: COc1ccc(C(=O)N(CC(C)(C)CCn2cc(CCCCCCCCCCCCCCCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

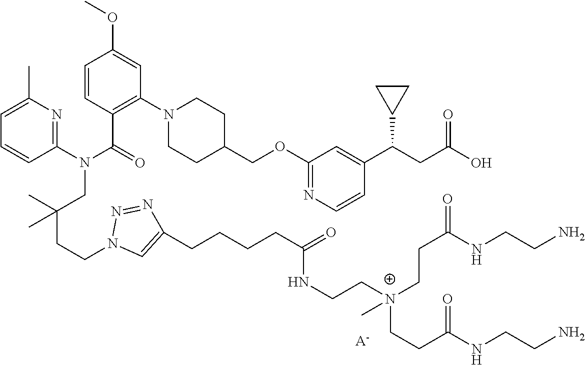

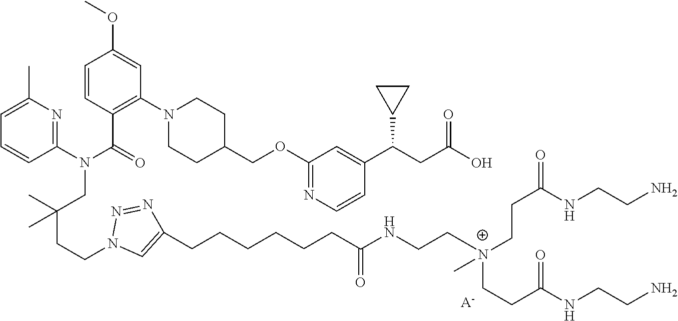

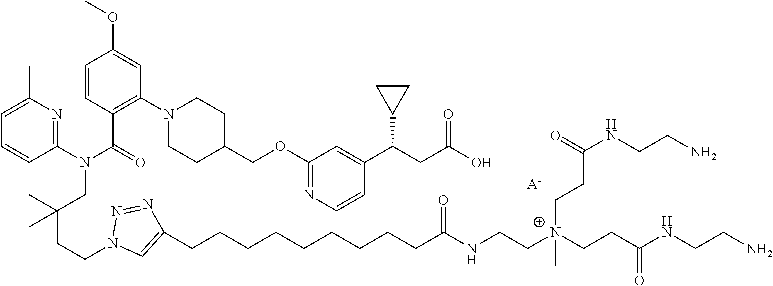

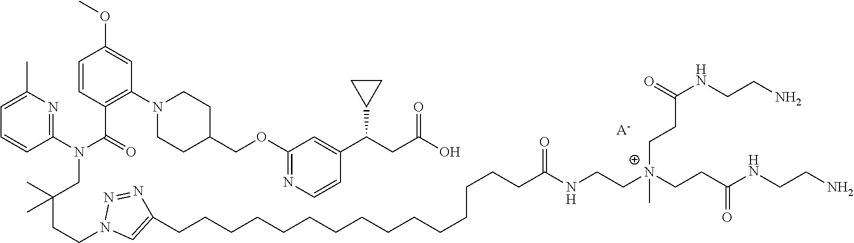

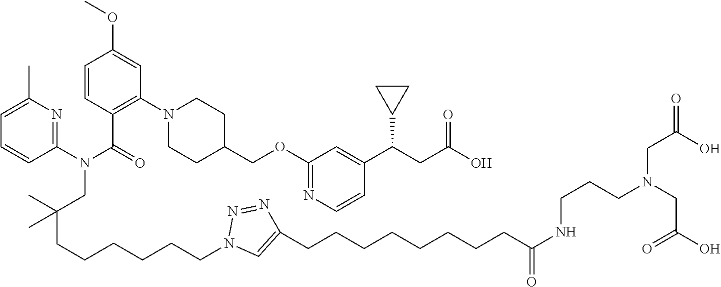 [SMILES: COc1ccc(C(=O)N(CC(C)(C)CCCCCCn2cc(CCCCCCCCC(=O)NCCCN(CC(=O)O)CC(=O)O)nn2)c2cccc(C)n2)c(N2CCC(COc3cc([C@@H](CC(=O)O)C4CC4)ccn3)CC2)c1]

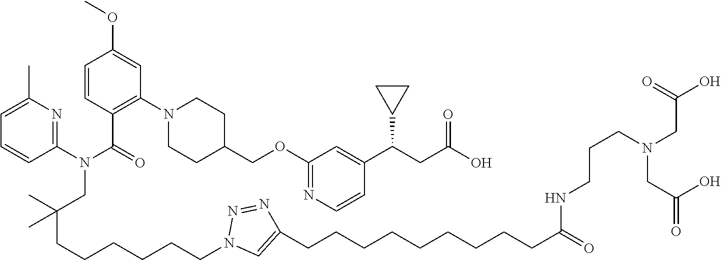 [SMILES: COc1ccc(C(=O)N(CC(C)(C)CCCCCCn2cc(CCCCCCCCCC(=O)NCCCN(CC(=O)O)CC(=O)O)nn2)c2cccc(C)n2)c(N2CCC(COc3cc([C@@H](CC(=O)O)C4CC4)ccn3)CC2)c1]

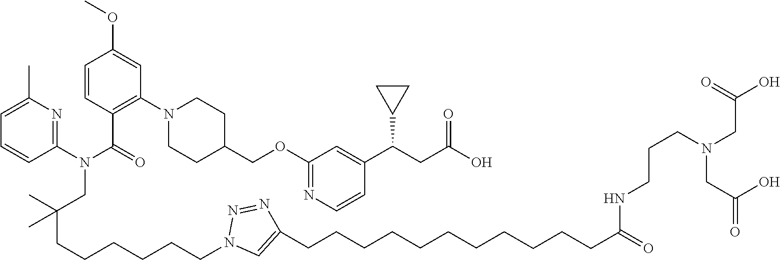 [SMILES: COc1ccc(C(=O)N(CC(C)(C)CCCCCCn2cc(CCCCCCCCCCCC(=O)NCCCN(CC(=O)O)CC(=O)O)nn2)c2cccc(C)n2)c(N2CCC(COc3cc([C@@H](CC(=O)O)C4CC4)ccn3)CC2)c1]

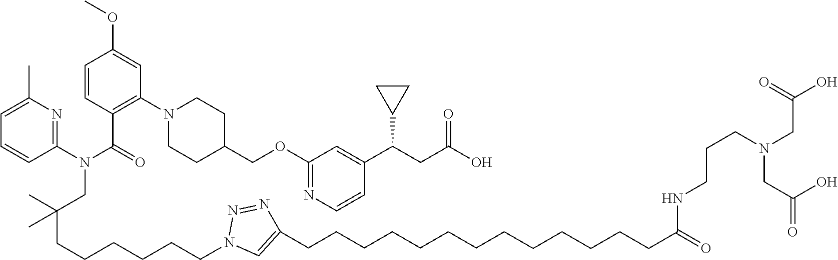 [SMILES: COc1ccc(C(=O)N(CC(C)(C)CCCCCCn2cc(CCCCCCCCCCCCCC(=O)NCCCN(CC(=O)O)CC(=O)O)nn2)c2cccc(C)n2)c(N2CCC(COc3cc([C@@H](CC(=O)O)C4CC4)ccn3)CC2)c1]

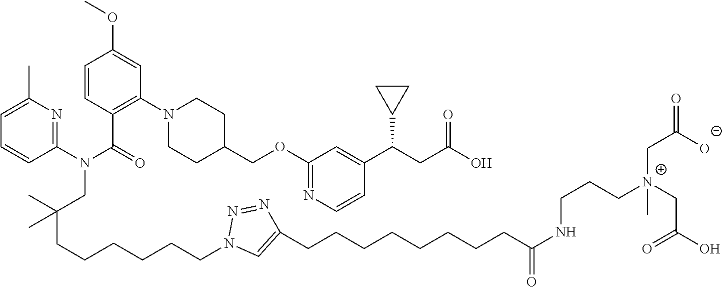 [SMILES: COc1ccc(C(=O)N(CC(C)(C)CCCCCCn2cc(CCCCCCCCC(=O)NCCC[N+](C)(CC(=O)[O-])CC(=O)O)nn2)c2cccc(C)n2)c(N2CCC(COc3cc([C@@H](CC(=O)O)C4CC4)ccn3)CC2)c1]

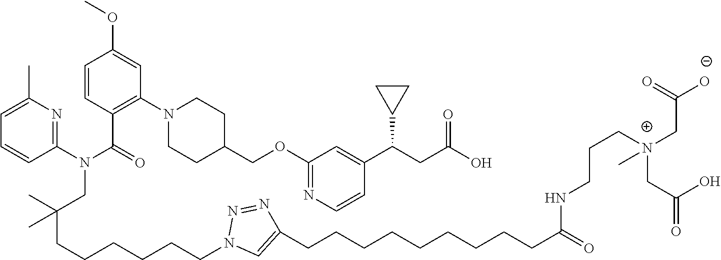 [SMILES: COc1ccc(C(=O)N(CC(C)(C)CCCCCCn2cc(CCCCCCCCCC(=O)NCCC[N+](C)(CC(=O)[O-])CC(=O)O)nn2)c2cccc(C)n2)c(N2CCC(COc3cc([C@@H](CC(=O)O)C4CC4)ccn3)CC2)c1]

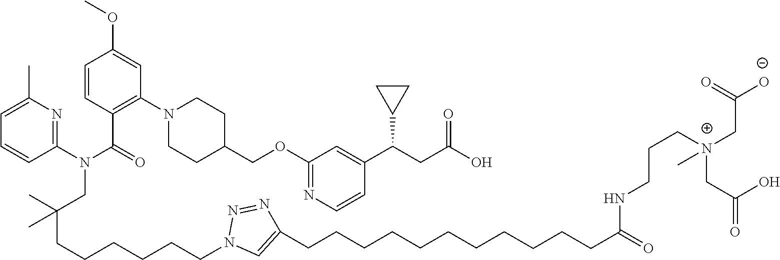 [SMILES: COc1ccc(C(=O)N(CC(C)(C)CCCCCCn2cc(CCCCCCCCCCCC(=O)NCCC[N+](C)(CC(=O)[O-])CC(=O)O)nn2)c2cccc(C)n2)c(N2CCC(COc3cc([C@@H](CC(=O)O)C4CC4)ccn3)CC2)c1]

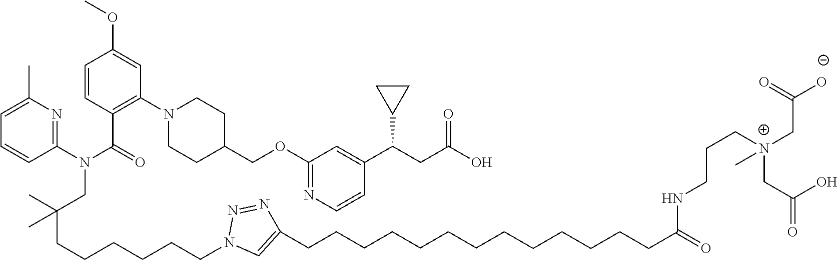 [SMILES: COc1ccc(C(=O)N(CC(C)(C)CCCCCCn2cc(CCCCCCCCCCCCCC(=O)NCCC[N+](C)(CC(=O)[O-])CC(=O)O)nn2)c2cccc(C)n2)c(N2CCC(COc3cc([C@@H](CC(=O)O)C4CC4)ccn3)CC2)c1]

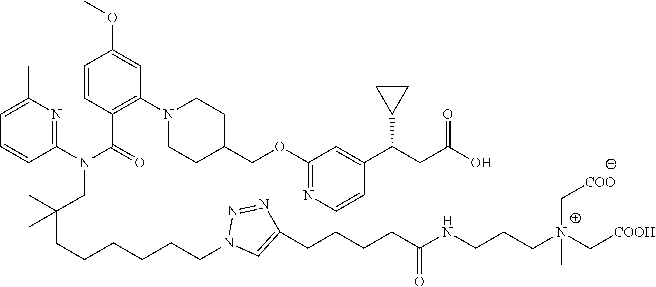

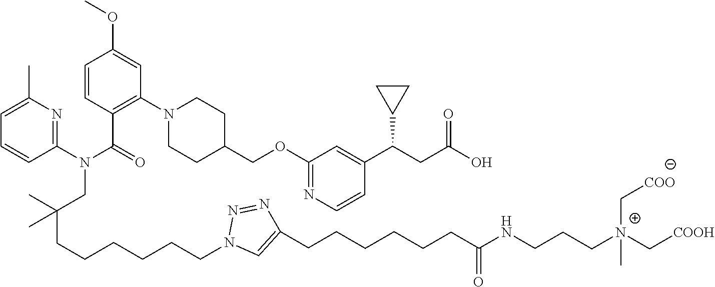

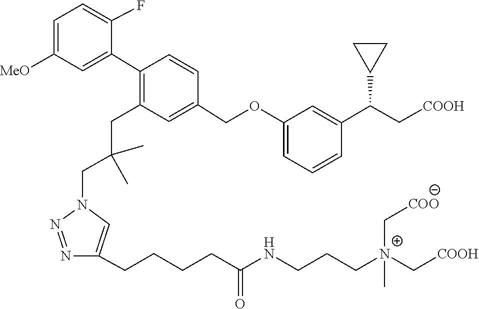

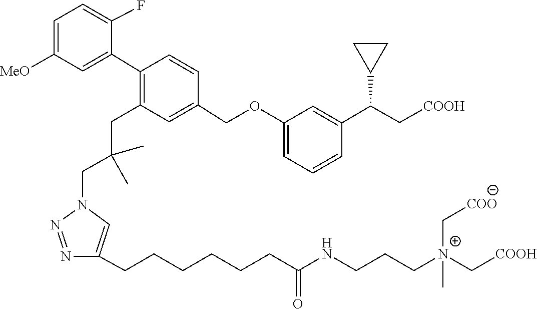

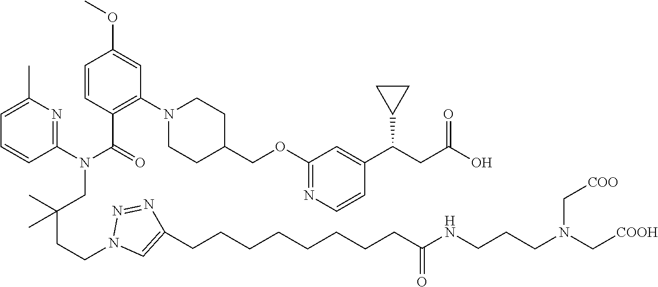

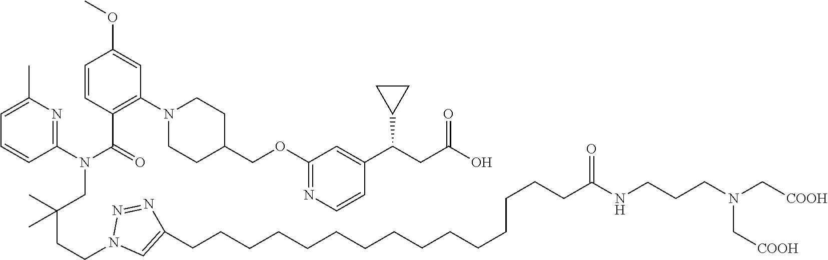 [SMILES: COc1ccc(C(=O)N(CC(C)(C)CCn2cc(CCCCCCCCCCCCCCCC(=O)NCCCN(CC(=O)O)CC(=O)O)nn2)c2cccc(C)n2)c(N2CCC(COc3cc([C@@H](CC(=O)O)C4CC4)ccn3)CC2)c1]

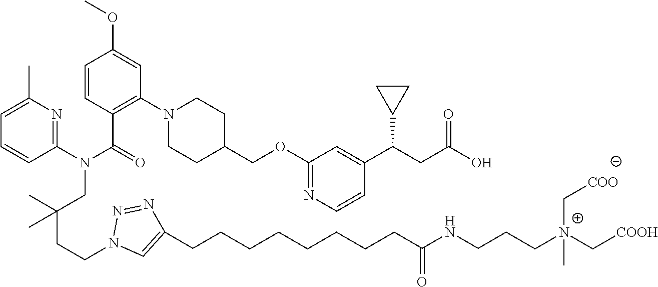

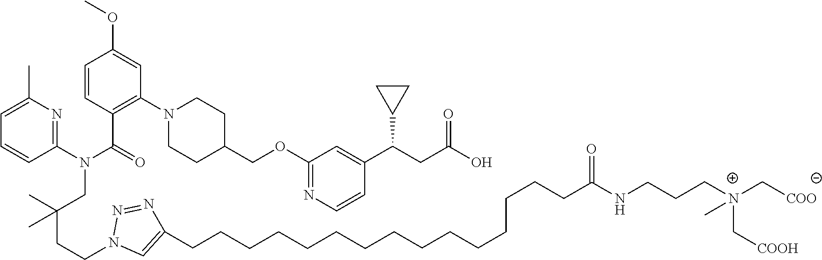

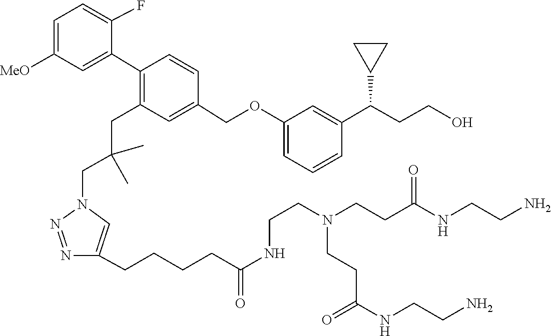 [SMILES: COc1ccc(F)c(-c2ccc(COc3cccc([C@@H](CCO)C4CC4)c3)cc2CC(C)(C)Cn2cc(CCCCC(=O)NCCN(CCC(=O)NCCN)CCC(=O)NCCN)nn2)c1]

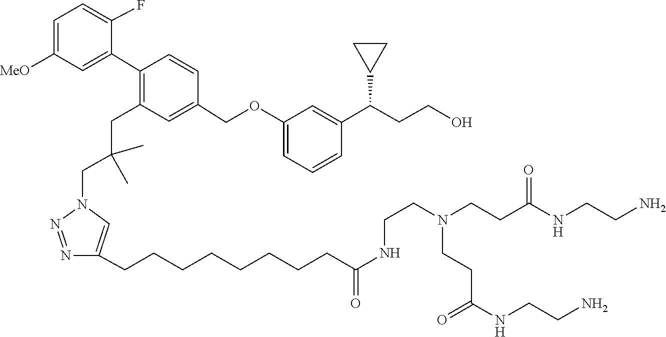 [SMILES: COc1ccc(F)c(-c2ccc(COc3cccc([C@@H](CCO)C4CC4)c3)cc2CC(C)(C)Cn2cc(CCCCCCCCC(=O)NCCN(CCC(=O)NCCN)CCC(=O)NCCN)nn2)c1]

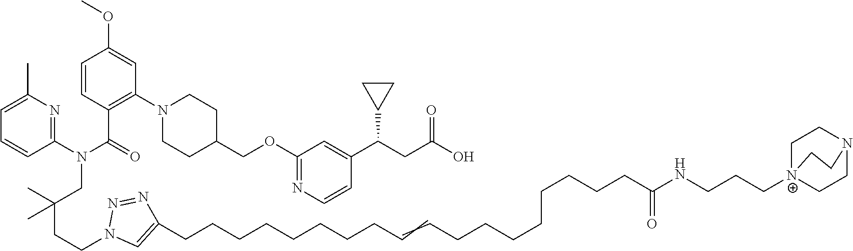 [SMILES: COc1ccc(C(=O)N(CC(C)(C)CCn2cc(CCCCCCCCC=CCCCCCCCCC(=O)NCCC[N+]34CCN(CC3)CC4)nn2)c2cccc(C)n2)c(N2CCC(COc3cc([C@@H](CC(=O)O)C4CC4)ccn3)CC2)c1]

In some embodiments, the compound of any one of the compounds in Table 1 can be present in a form of a pharmaceutically acceptable salt.

In some embodiments, the compound of any one of the compounds in Table 1 can be present in a form of a pharmaceutically acceptable ester, or a pharmaceutically acceptable salt thereof.

In some embodiments, the present disclosure also provides a compound according to any of Examples 1-221, or a pharmaceutically acceptable salt thereof. A compound according to any of Examples 1-221 should be understood as the same compound as drawn in Examples 1-221 without considering its salt form and/or any counterion(s); the compound may exist in a different salt form and/or containing a different counterion(s).

In some embodiments, the present disclosure also provides a compound according to any of GPR-1, GPR-2, GPR-2B, GPR-3, or GPR-3B, or a pharmaceutically acceptable salt or ester thereof.

As discussed herein, the present inventors believe that the cLogP of L^{A} and T^{A} and the length of L^{A}, but not their exact identities, are important in achieving potent GPR40 agonist activity. The following Tables A-1a, A-1b, A-2a, A-2b, A-3a, A-3b, A-4a, A-4b, A-5a, A-5b, A-6a, A-6b, A-7 and A-8 further support the role of cLogP of L^{A} and T^{A} for representative compounds of this disclosure. For example, when the cLogP of L^{A} drops below certain point, the GPR40 activities drop significantly (as shown by the large increase of EC50 values). The EC50 values in the tables were obtained by following methods described in Biological Example 1.

TABLE A-1a
Structure-Property Relationship of Examples according to A-1a

| | | | | | | A-1a |
|---|---|---|---|---|---|---|
| 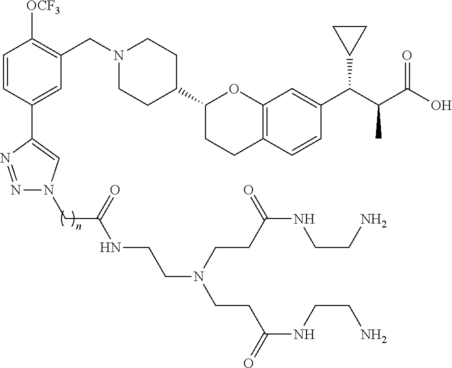 | | | | | | |
| 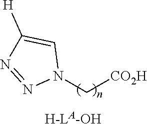 | | | | | | |
| 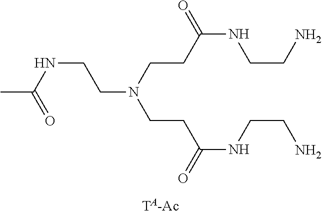 [SMILES: CC(=O)NCCN(CCC(=O)NCCN)CCC(=O)NCCN ; "TA-Ac"] | | | | | | |
| n | 17 | 15 | 14 | 13 | 11 | 9 |
| Example # | 6 | 39 | 71 | 5 | 36 | 77 |
| EC50 (nM) | 1.21 | 0.83 | 6.71 | 15.2 | 403 | >10000 |
| H-L^{A}-OH cLogP | 6.52 | 5.46 | 4.93 | 4.40 | 3.32 | 2.29 |
| T^{A}-Ac cLogP | −1.84 | −1.84 | −1.84 | −1.84 | −1.84 | −1.84 |

TABLE A-1b
Structure-Property Relationship of Examples according to A-1b

| | | | | | | A-1b |
|---|---|---|---|---|---|---|
| 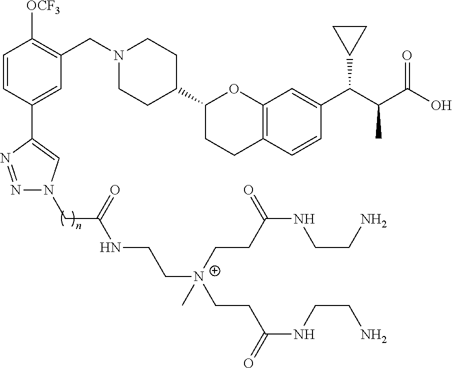 | | | | | | |
| 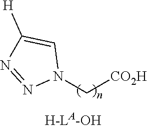 | | | | | | |
| 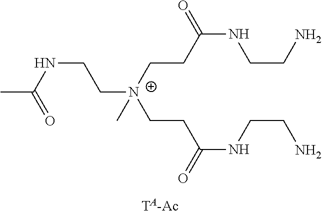 [SMILES: CC(=O)NCC[N+](C)(CCC(=O)NCCN)CCC(=O)NCCN ; "TA-Ac"] | | | | | | |
| n | 17 | 15 | 14 | 13 | 11 | 9 |
| Example # | 7 | 84 | 72 | 41 | 83 | 82 |
| EC50 (nM) | 0.65 | 0.74 | 6.74 | 10.1 | 650 | >10000 |
| H-L^{A}-OH cLogP | 6.52 | 5.46 | 4.93 | 4.40 | 3.32 | 2.29 |
| T^{A}-Ac cLogP | −3.19 | −3.19 | −3.19 | −3.19 | −3.19 | −3.19 |

TABLE A-2a
Structure-Property Relationship of Examples according to A-2a*

| | | | | | | A-2a |
|---|---|---|---|---|---|---|
| 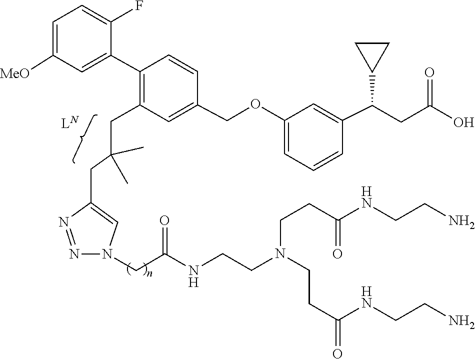 | | | | | | |
| 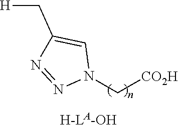 | | | | | | |
| 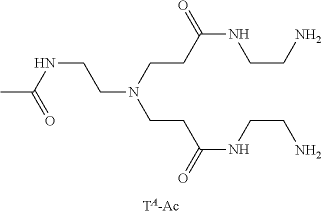 [SMILES: CC(=O)NCCN(CCC(=O)NCCN)CCC(=O)NCCN ; "TA-Ac"] | | | | | | |
| n | 17 | 15 | 14 | 13 | 11 | 9 |
| Example # | 23 | 89 | 52 | 21 | 92 | 135 |
| EC50 (nM) | 0.96 | 2.19 | 26 | 49 | 212 | >3700 |
| H-L^{A}-OH cLogP | 6.79 | 5.73 | 5.20 | 4.67 | 3.61 | 2.55 |
| T^{A}-Ac cLogP | −1.84 | −1.84 | −1.84 | −1.84 | −1.84 | −1.84 |

*It should be noted that the designation of L^{N} is only one way of dissecting the molecules and not limiting, the same should be understood in other tables herein with the L^{N} designations. When L^{N} is included as part of the L^{A}, it would be clear that the cLogP values may change, but the trend observed would remain the same.

TABLE A-2b
Structure-Property Relationship of Examples according to A-2b

| | | | | | | A-2b |
|---|---|---|---|---|---|---|
| 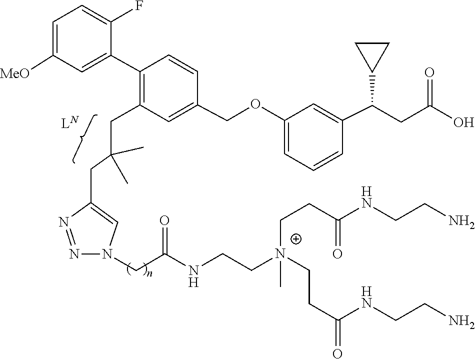 | | | | | | |
| 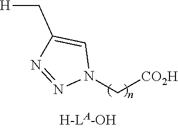 | | | | | | |
| 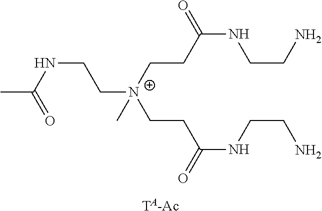 [SMILES: CC(=O)NCC[N+](C)(CCC(=O)NCCN)CCC(=O)NCCN ; "TA-Ac"] | | | | | | |
| n | 17 | 15 | 14 | 13 | 11 | 9 |
| Example # | 24 | 90 | 102 | 22 | 97 | 96 |
| EC50 (nM) | 2.04 | 2.60 | 4.05 | 49 | 328 | 1055 |
| H-L^{A}-OH cLogP | 6.79 | 5.73 | 5.20 | 4.67 | 3.61 | 2.55 |
| T^{A}-Ac cLogP | −3.19 | −3.19 | −3.19 | −3.19 | −3.19 | −3.19 |

TABLE A-3a
Structure-Property Relationship of Examples according to A-3a

| | | | | | | | | A-3a |
|---|---|---|---|---|---|---|---|---|
| 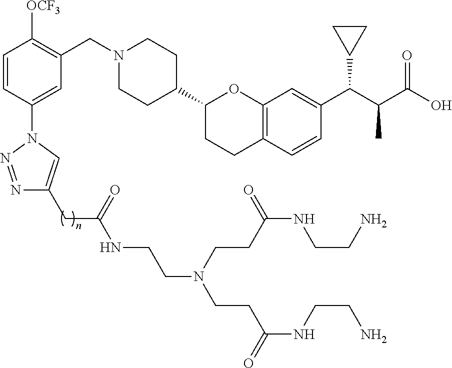 | | | | | | | | |
| 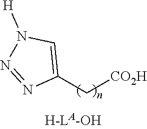 | | | | | | | | |
| 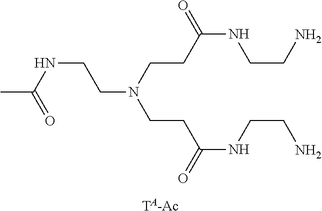 [SMILES: CC(=O)NCCN(CCC(=O)NCCN)CCC(=O)NCCN ; "TA-Ac"] | | | | | | | | |
| n | 17 | 15 | 14 | 13 | 12 | 10 | 9 | 8 |
| Example # | 3 | 185 | 183 | 78 | 1 | 79 | 193 | 80 |
| EC50 (nM) | 1.1 | 0.05 | 0.16 | 1.40 | 5.46 | 183 | 413 | >5000 |
| H-L^{A}-OH cLogP | 7.13 | 6.07 | 5.54 | 5.01 | 4.49 | 3.43 | 2.90 | 2.37 |
| T^{A}-Ac cLogP | −1.84 | −1.84 | −1.84 | −1.84 | −1.84 | −1.84 | −1.84 | −1.84 |

TABLE A-3b
Structure-Property Relationship of Examples according to A-3b

| | | | | | | | | A-3b |
|---|---|---|---|---|---|---|---|---|
| 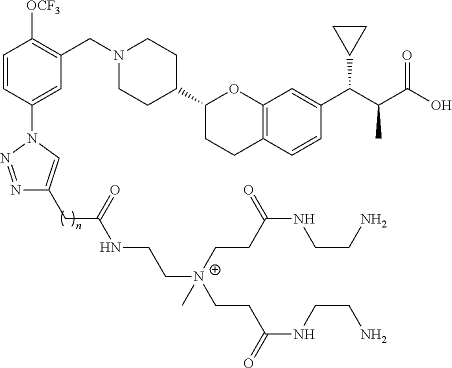 | | | | | | | | |
| 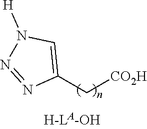 | | | | | | | | |
| 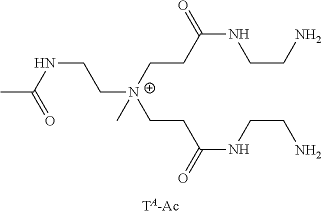 [SMILES: CC(=O)NCC[N+](C)(CCC(=O)NCCN)CCC(=O)NCCN ; "TA-Ac"] | | | | | | | | |
| n | 17 | 15 | 14 | 13 | 12 | 10 | 9 | 8 |
| Example # | 4 | 186 | 184 | 62 | 2 | 86 | 194 | 69 |
| EC50 (nM) | 0.31 | 0.05 | 0.16 | 0.61 | 9.46 | 195 | 114 | >10000 |
| H-L^{A}-OH cLogP | 7.13 | 6.07 | 5.54 | 5.01 | 4.49 | 3.43 | 2.90 | 2.37 |
| T^{A}-Ac cLogP | −3.19 | −3.19 | −3.19 | −3.19 | −3.19 | −3.19 | −3.19 | −3.19 |

TABLE A-4a
Structure-Property Relationship of Examples according to A-4a

| | | | | | | | | | A-4a |
|---|---|---|---|---|---|---|---|---|---|
| 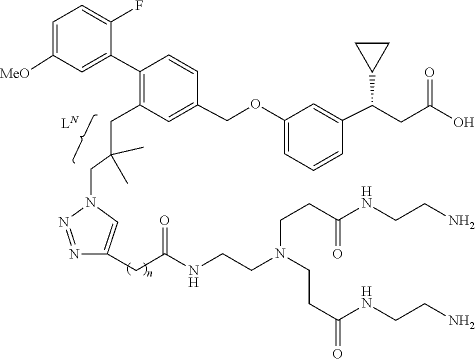 | | | | | | | | | |
| 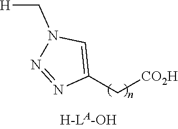 | | | | | | | | | |
| 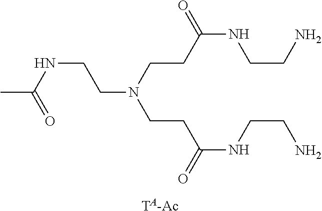 [SMILES: CC(=O)NCCN(CCC(=O)NCCN)CCC(=O)NCCN ; "TA-Ac"] | | | | | | | | | |
| n | 17 | 15 | 14 | 13 | 12 | 10 | 9 | 8 | 6 |
| Example # | 18 | 187 | 49 | 55 | 16 | 56 | 191 | 220 | 94 |
| EC50 (nM) | 2.25 | 1.34 | 19.9 | 138 | 460 | 1760 | 204 | >10000 | 1952 |
| H-L^{A}-OH cLogP | 6.79 | 5.73 | 5.20 | 4.67 | 4.14 | 3.08 | 2.55 | 2.03 | 0.97 |
| T^{A}-Ac cLogP | −1.84 | −1.84 | −1.84 | −1.84 | −1.84 | −1.84 | −1.84 | −1.84 | −1.84 |

TABLE A-4b
Structure-Property Relationship of Examples according to A-4b

| | | | | | | | | | A-4b |
|---|---|---|---|---|---|---|---|---|---|
| 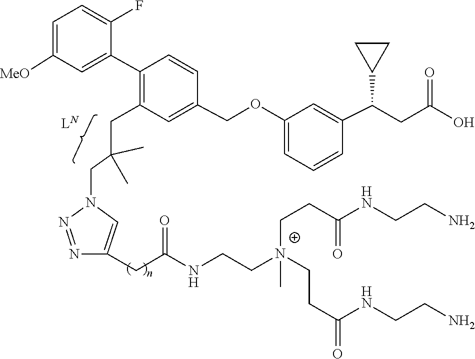 | | | | | | | | | |
| 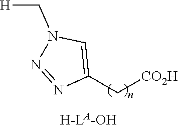 | | | | | | | | | |
| 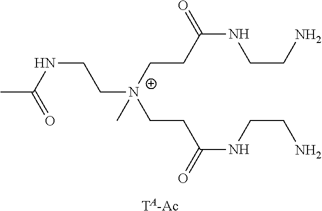 [SMILES: CC(=O)NCC[N+](C)(CCC(=O)NCCN)CCC(=O)NCCN ; "TA-Ac"] | | | | | | | | | |
| n | 17 | 15 | 14 | 13 | 12 | 10 | 9 | 8 | 6 |
| Example # | 19 | 188 | 58 | 57 | 17 | 59 | 192 | 144 | 95 |
| EC50 (nM) | 1.66 | 1.53 | 8.14 | 93.1 | 152 | 1255 | 250 | 5166 | >10000 |
| H-L^{A}-OH cLogP | 6.79 | 5.73 | 5.20 | 4.67 | 4.14 | 3.08 | 2.55 | 2.03 | 0.97 |
| T^{A}-Ac cLogP | −3.19 | −3.19 | −3.19 | −3.19 | −3.19 | −3.19 | −3.19 | −3.19 | −3.19 |

TABLE A-5a
Structure-Property Relationship of Examples according to A-5a

| | | | | | | | A-5a |
|---|---|---|---|---|---|---|---|
| 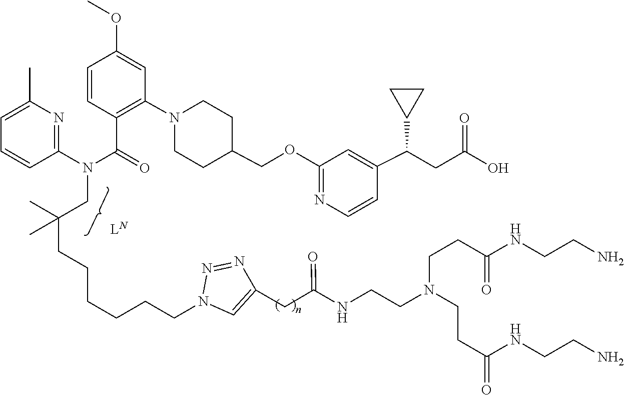 | | | | | | | |
| 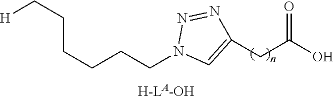 | | | | | | | |
| 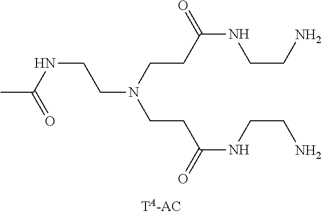 [SMILES: CC(=O)NCCN(CCC(=O)NCCN)CCC(=O)NCCN] | | | | | | | |
| n | 13 | 12 | 10 | 9 | 8 | 6 | 4 |
| Example # | 189 | 68 | 74 | | 75 | 98 | 100 |
| EC50 (nM) | 0.05 | 1.79 | 2.99 | | 1.92 | 3.54 | 163 |
| H-L^{A}-OH cLogP | 7.32 | 6.79 | 5.73 | 5.20 | 4.67 | 3.61 | 2.55 |
| T^{A}-Ac cLogP | −1.84 | −1.84 | −1.84 | −1.84 | −1.84 | −1.84 | −1.84 |

TABLE A-5b
Structure-Property Relationship of Examples according to A-5b

| | | | | | | | A-5b |
|---|---|---|---|---|---|---|---|
| 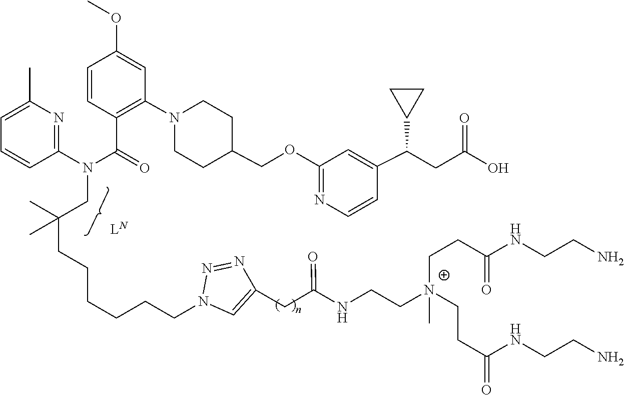 | | | | | | | |
| 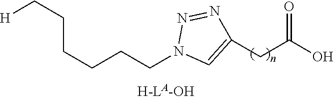 | | | | | | | |
| 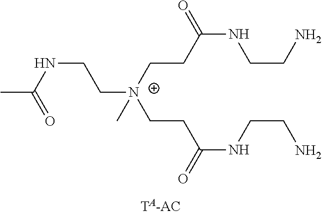 [SMILES: CC(=O)NCC[N+](C)(CCC(=O)NCCN)CCC(=O)NCCN] | | | | | | | |
| n | 13 | 12 | 10 | 9 | 8 | 6 | 4 |
| Example # | 190 | 43 | 54 | | 53 | 99 | 101 |
| EC50 (nM) | 0.05 | 0.21 | 1.71 | | 3.81 | 4.58 | 192 |
| H-L^{A}-OH cLogP | 7.32 | 6.79 | 5.73 | 5.20 | 4.67 | 3.61 | 2.55 |
| T^{A}-Ac cLogP | −3.19 | −3.19 | −3.19 | −3.19 | −3.19 | −3.19 | −3.19 |

TABLE A-6a
Structure-Property Relationship of Examples according to A-6a

| | | | | | | | | | A-6a |
|---|---|---|---|---|---|---|---|---|---|
| 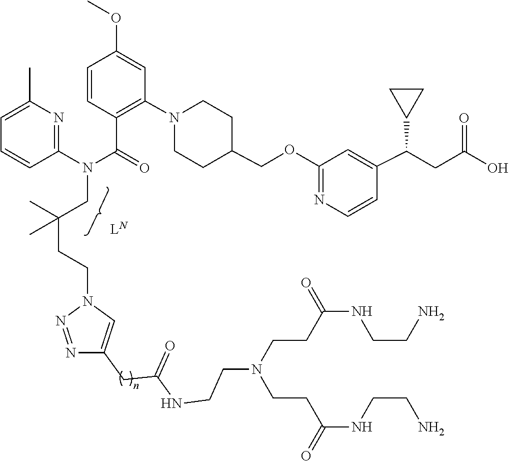 | | | | | | | | | |
| 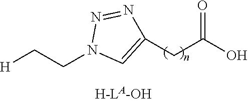 | | | | | | | | | |
| 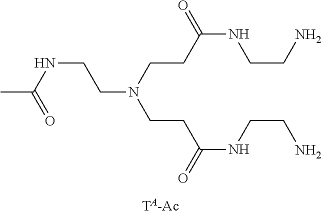 [SMILES: CC(=O)NCCN(CCC(=O)NCCN)CCC(=O)NCCN ; "TA-Ac"] | | | | | | | | | |
| n | 15 | 14 | 13 | 12 | 10 | 9 | 8 | 6 | 4 |
| Example # | 198 | 118 | 116 | 114 | 112 | 197 | 110 | 196 | 315 |
| EC50 (nM) | 0.17 | 0.33 | 0.21 | 0.42 | 14.9 | 84.3 | 618 | 9872 | >10000 |
| H-L^{A}-OH cLogP | 6.26 | 5.73 | 5.2 | 4.67 | 3.61 | 3.08 | 2.55 | 1.50 | 1.44 |
| T^{A}-Ac cLogP | −1.84 | −1.84 | −1.84 | −1.84 | −1.84 | −1.84 | −1.84 | −1.84 | −1.84 |

TABLE A-6b
Structure-Property Relationship of Examples according to A-6b

| | | | | | | | | | A-6b |
|---|---|---|---|---|---|---|---|---|---|
| 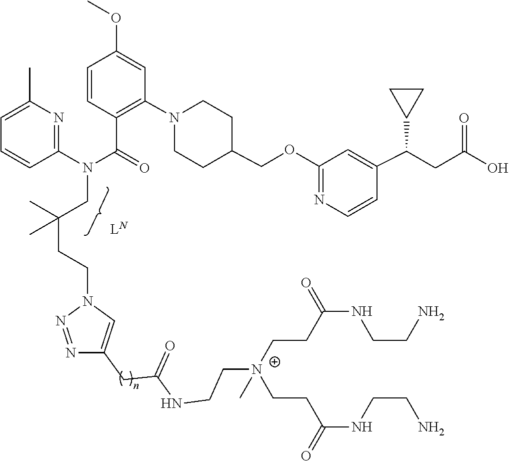 | | | | | | | | | |
| 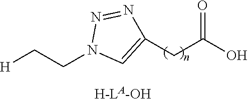 | | | | | | | | | |
| 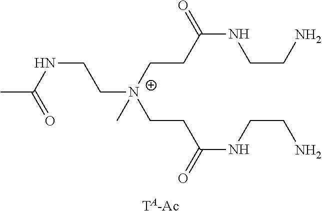 [SMILES: CC(=O)NCC[N+](C)(CCC(=O)NCCN)CCC(=O)NCCN ; "TA-Ac"] | | | | | | | | | |
| n | 15 | 14 | 13 | 12 | 10 | 9 | 8 | 6 | 4 |
| Example # | 202 | 119 | 117 | 115 | 113 | 201 | 111 | 200 | 199 |
| EC50 (nM) | 0.16 | 0.10 | 0.29 | 0.51 | 6.27 | 45 | 307 | >10000 | >10000 |
| H-L^{A}-OH cLogP | 6.26 | 5.73 | 5.20 | 4.67 | 3.61 | 3.08 | 2.55 | 1.50 | 1.44 |
| T^{A}-Ac cLogP | −3.19 | −3.19 | −3.19 | −3.19 | −3.19 | −3.19 | −3.19 | −3.19 | −3.19 |

TABLE A-7a
Structure-Property Relationship of Examples according to A-7a

| | | | | | | | A-7a |
|---|---|---|---|---|---|---|---|
| 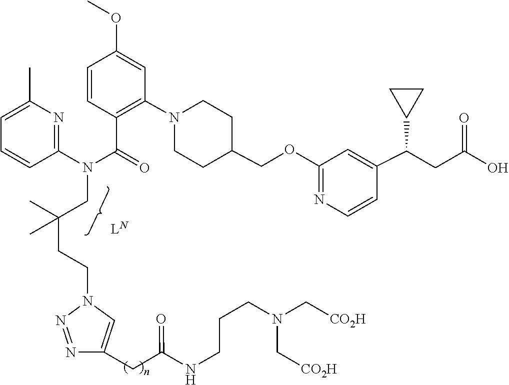 | | | | | | | |
| 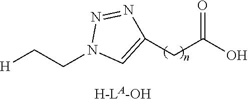 | | | | | | | |
| 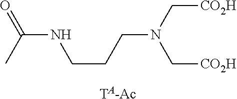 [SMILES: CC(=O)NCCCN(CC(=O)O)CC(=O)O ; "TA-Ac"] | | | | | | | |
| n | 16 | 15 | 14 | 13 | 11 | 9 | 8 |
| Example # | 132 | 216 | 130 | 128 | 126 | 124 | 215 |
| EC50 (nM) | 0.19 | 0.48 | 1.25 | 1.33 | 5.81 | 226 | 1357 |
| H-L^{A}-OH cLogP | 6.79 | 6.26 | 5.73 | 5.20 | 4.14 | 3.08 | 2.55 |
| T^{A}-Ac cLogP | −1.56 | −1.56 | −1.56 | −1.56 | −1.56 | −1.56 | −1.56 |

TABLE A-7b
Structure-Property Relationship of Examples according to A-7b

| | | | | | | | A-7b |
|---|---|---|---|---|---|---|---|
| 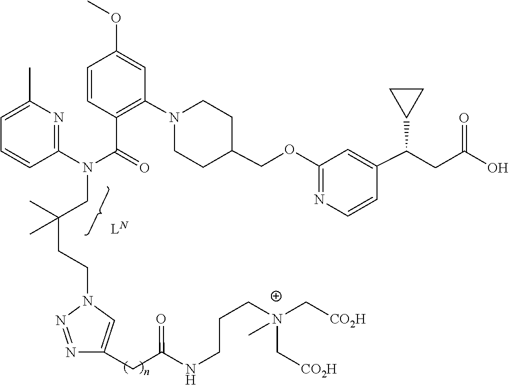 | | | | | | | |
| 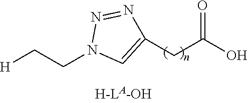 | | | | | | | |
| 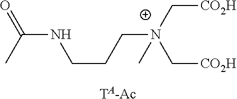 [SMILES: CC(=O)NCCC[N+](C)(CC(=O)O)CC(=O)O ; "TA-Ac"] | | | | | | | |
| n | 16 | 15 | 14 | 13 | 11 | 9 | 8 |
| Example # | 133 | 218 | 131 | 129 | 127 | 125 | 217 |
| EC50 (nM) | 0.08 | 0.44 | 0.38 | 0.88 | 6.36 | 617 | 683 |
| H-L^{A}-OH cLogP | 6.79 | 6.26 | 5.73 | 5.2 | 4.14 | 3.08 | 2.55 |
| T^{A}-Ac cLogP | −2.71 | −2.71 | −2.71 | −2.71 | −2.71 | −2.71 | −2.71 |

TABLE A-8
Structure-Property Relationship of Additional Examples

| | Corresponding Structure of H-L^{A}-OH | | | |
|---|---|---|---|---|
| Example No. | 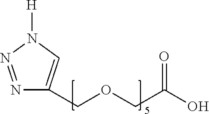 | | 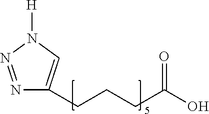 | |
| Example 173 | EC50: >10000 nM | H-L^{A}-OH cLogP: −1.54 | | |
| Example 185 | | | EC50: 0.05 nM | H-L^{A}-OH cLogP: 6.07 |
| Example 176 | EC50: >10000 nM | H-L^{A}-OH cLogP: −1.54 | | |
| Example 186 | | | EC50: 0.05 nM | H-L^{A}-OH cLogP: 6.07 |
| | 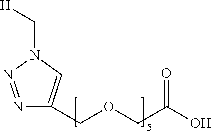 | | 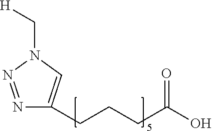 | |
| Example 167 | EC50: >10000 nM | H-L^{A}-OH cLogP: −1.89 | | |
| Example 187 | | | EC50: 1.34 nM | H-L^{A}-OH cLogP: 5.73 |
| Example 170 | EC50: >200 nM | H-L^{A}-OH cLogP: −1.89 | | |
| Example 188 | | | EC50: 1.53 nM | H-L^{A}-OH cLogP: 5.73 |
| | 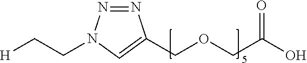 | | 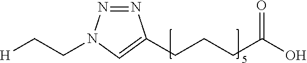 | |
| Example 161 | EC50: 2200 nM | H-L^{A}-OH cLogP: −1.36 | | |
| Example 198 | | | EC50: 0.17 nM | H-L^{A}-OH cLogP: 6.26 |
| Example 164 | EC50: 1331 nM | H-L^{A}-OH cLogP: −1.36 | | |
| Example 202 | | | EC50: 0.16 nM | H-L^{A}-OH cLogP: 6.26 |
| | 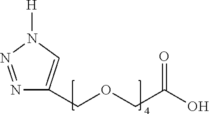 | | 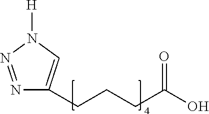 | |
| Example 172 | EC50: 5240 nM | H-L^{A}-OH cLogP: −1.37 | | |
| Example 1 | | | EC50: 5.46 nM | H-L^{A}-OH cLogP: 4.49 |
| Example 175 | EC50: >10000 nM | H-L^{A}-OH cLogP: −1.37 | | |
| Example 2 | | | EC50: 9.46 nM | H-L^{A}-OH cLogP: 4.49 |
| | 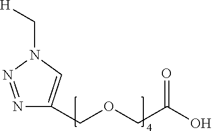 | | 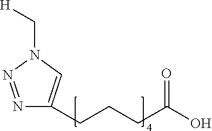 | |
| Example 166 | EC50: >10000 nM | H-L^{A}-OH cLogP: −1.71 | | |
| Example 16 | | | EC50: 706 nM | H-L^{A}-OH cLogP: 4.14 |
| Example 169 | EC50: >10000 nM | H-L^{A}-OH cLogP: −1.71 | | |
| Example 17 | | | EC50: 169 nM | H-L^{A}-OH cLogP: 4.14 |
| | 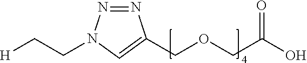 | | 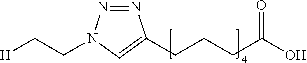 | |
| Example 160 | EC50: 1365 nM | H-L^{A}-OH cLogP: −1.18 | | |
| Example 114 | | | EC50: 0.42 nM | H-L^{A}-OH cLogP: 4.67 |
| Example 163 | EC50: >5000 nM | H-L^{A}-OH cLogP: −1.18 | | |
| Example 115 | | | EC50: 0.51 nM | H-L^{A}-OH cLogP: 4.67 |
| | 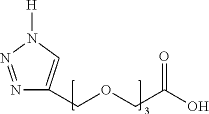 | | 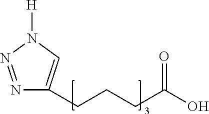 | |
| Example 171 | EC50: >10000 nM | H-L^{A}-OH cLogP: −1.19 | | |
| Example 193 | | | EC50: 413 nM | H-L^{A}-OH cLogP: 2.90 |
| Example 174 | EC50: >10000 nM | H-L^{A}-OH cLogP: −1.19 | | |
| Example 194 | | | EC50: 94 nM | H-L^{A}-OH cLogP: 2.90 |
| | 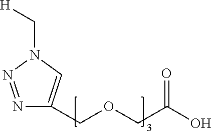 | | 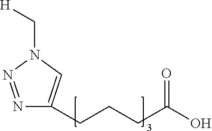 | |
| Example 165 | EC50: >10000 nM | H-L^{A}-OH cLogP: −1.54 | | |
| Example 191 | | | EC50: 209 nM | H-L^{A}-OH cLogP: 2.55 |
| Example 168 | EC50: >10000 nM | H-L^{A}-OH cLogP: −1.54 | | |
| Example 192 | | | EC50: 251 nM | H-L^{A}-OH cLogP: 2.55 |
| | 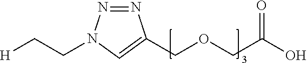 | | 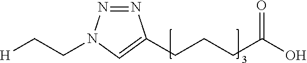 | |
| Example 159 | EC50: >6000 nM | H-L^{A}-OH cLogP: −1.01 | | |
| Example 197 | | | EC50: 84 nM | H-L^{A}-OH cLogP: 3.08 |
| Example 162 | EC50: 1252 nM | H-L^{A}-OH cLogP: −1.01 | | |
| Example 201 | | | EC50: 45 nM | H-L^{A}-OH cLogP: 3.08 |

The compounds herein can be prepared by those skilled in the art in view of the present disclosure. Exemplary synthesis are shown in the Examples section, such as those shown in the schemes in the Examples section, which can be adopted by those skilled in the art to synthesize other compounds of the present disclosure.

As exemplified herein, compounds of present disclosure can be typically prepared by a coupling reaction to link the residue of a GPR40 agonist with a hydrophilic molecule. Suitable coupling reactions are not particularly limited, which will depend on the structural features of the compound.

For example, in some embodiments, the compound contains a triazole, and can be synthesized by coupling an azide containing compound with an alkyne containing compound under click-chemistry conditions. Click-chemistry is well known in the art and suitable reaction conditions include those known in the art and those exemplified herein. Using compounds of Formula II-5 or III-5 as an example, when one instance of X is a triazole, the compounds can be synthesized by coupling appropriate alkynes and azides under click-chemistry conditions to form the target compounds of Formula II-5 or III-5, see scheme A-1 or B-1 below. For example, in Scheme A-1, a compound of S-1 can be coupled with a compound of S-2 under click-chemistry conditions, wherein J¹⁰ is alkyne and J¹¹ is N3 or J¹⁰ is N3 and J¹¹ is alkyne to form a compound of Formula II-5, wherein one instance of X is triazole, and wherein a+b+1 equals to m1. The variables L¹⁰, L^{N}, R¹¹, R¹², R¹³, R^{A}, R^{C}, X, Y, Z, G¹, G³, G⁴, A, r, m1, p1, and p2 in Scheme A-1 include any of those described and preferred herein in connection with Formula II in any combinations, provided that one instance of X in Formula II-5 is triazole formed by the coupling of J¹⁰ and J¹¹. Similarly, in Scheme B-1, a compound of S-3 can be coupled with a compound of S-2 under click-chemistry conditions, wherein J¹⁰ is alkyne and J¹¹ is N3 or J¹⁰ is N3 and J¹¹ is alkyne to form a compound of Formula III-5, wherein one instance of X is triazole, and wherein a+b+1 equals to m1. The variables L¹⁰, L^{N}, R¹¹, R¹², R¹⁸, R^{A}, R^{C}, ring A, X, Y, Z, G¹, G³, G⁴, A, r, m1, p1, and p2 in Scheme B-1 include any of those described and preferred herein in connection with Formula III in any combinations, provided that one instance of X in Formula III-5 is triazole formed by the coupling of J¹⁰ and J¹¹.

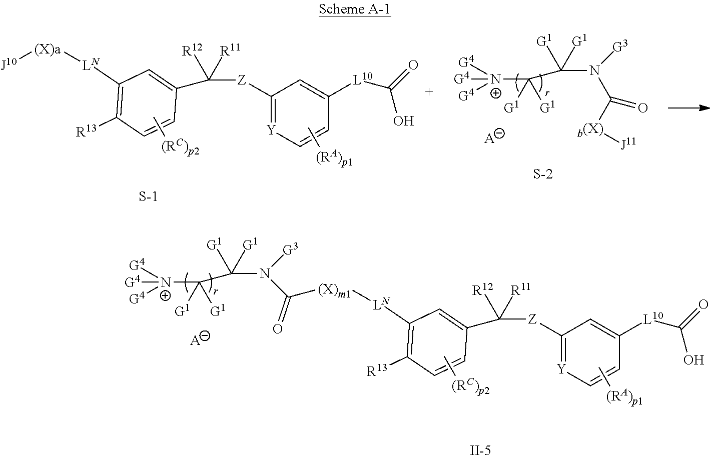

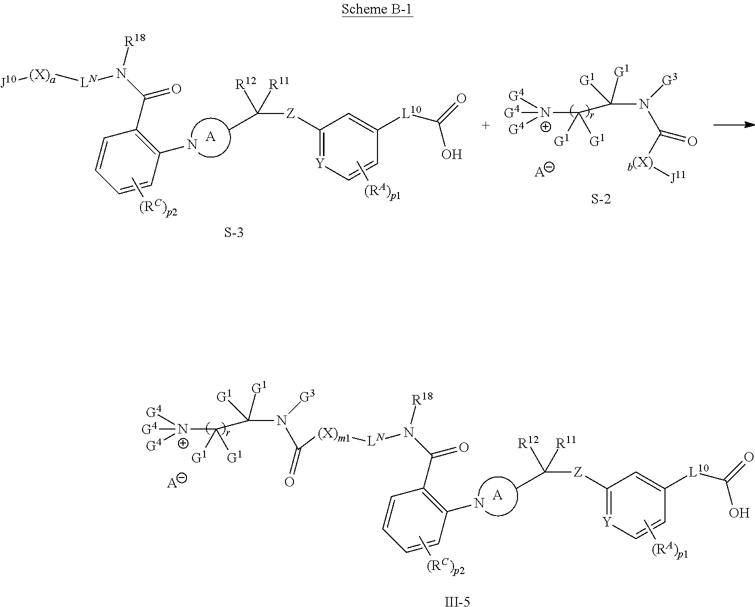

In some embodiments, an amide coupling can be used to link the residue of a GPR40 agonist with a hydrophilic molecule. For example, a compound of Formula II-4 can be prepared according to the synthetic sequence shown in Scheme A-2, which can convert an acid of S-4 with an amine of S-5 under amide coupling conditions, which can then be followed by deprotection to provide the compound of Formula II-4. The Pg1 in S-4 refers to a carboxylic acid protecting group, such as a tert-butyl group. Similarly, as shown in Scheme A-3, a compound of Formula II-5 can be prepared by coupling the acid of S-4 with an amine of S-6 under amide coupling conditions, which can then be followed by deprotection to provide the compound of Formula II-5. The variables L¹⁰, L^{N}, R¹¹, R¹², R¹³, R^{A}, R^{C}, X, Y, Z, G¹, G², G³, G⁴, A, r, m1, p1, and p2 in Scheme A-2 or A-3 include any of those described and preferred herein in connection with Formula II in any combinations.

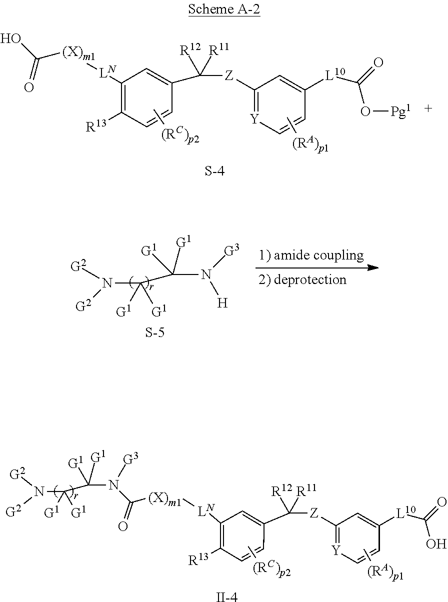

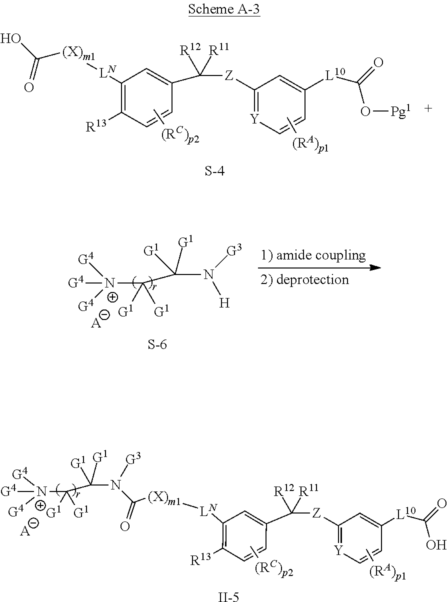

Similarly, a compound of Formula III-4 can be prepared according to the synthetic sequence shown in Scheme B-2, which can convert an acid of S-7 with an amine of S-5 under amide coupling conditions, which can then be followed by deprotection to provide the compound of Formula III-4. The Pg1 in S-7 refers to a carboxylic acid protecting group, such as a tert-butyl group. Similarly, as shown in Scheme B-3, a compound of Formula III-5 can be prepared by coupling the acid of S-8 with an amine of S-6 under amide coupling conditions, which can then be followed by deprotection to provide the compound of Formula III-5. The variables L¹⁰, L^{N}, R¹¹, R¹², R¹⁸, R^{A}, R^{C}, ring A, X, Y, Z, G¹, G², G³, G⁴, A, r, m1, p1, and p2 in Scheme B-2 or B-3 include any of those described and preferred herein in connection with Formula III in any combinations.

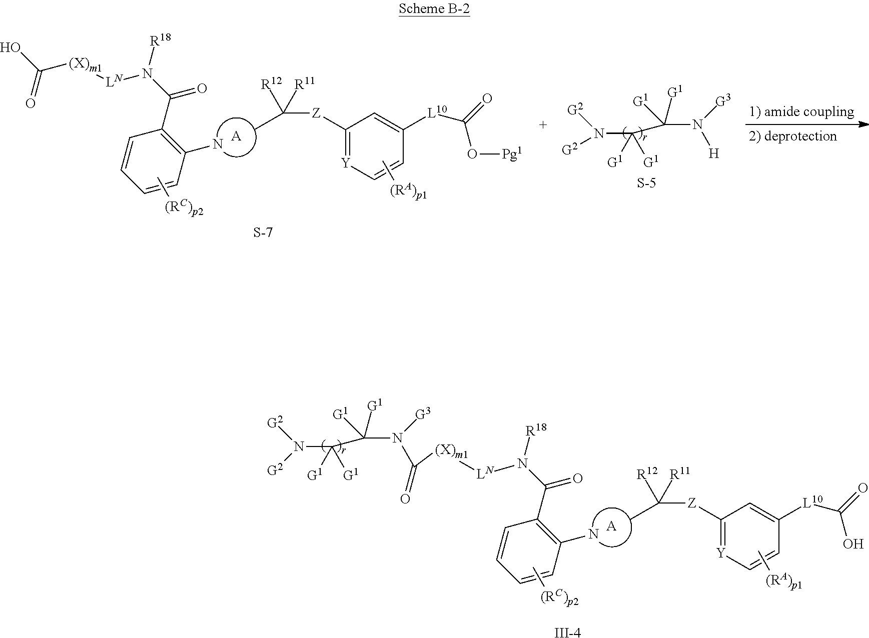

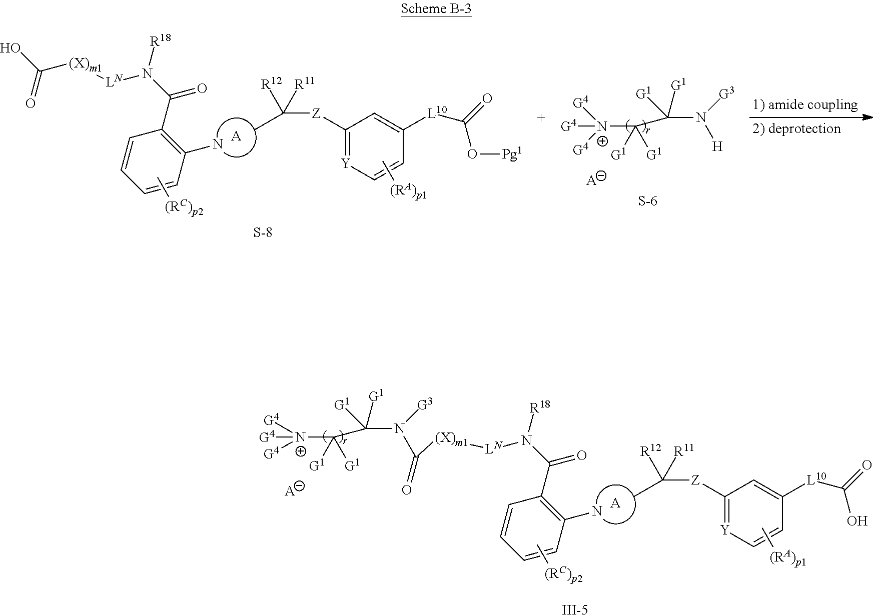

Similarly, compounds of Formula I can be prepared through click-chemistry or amide coupling reactions. For example, as shown in Scheme C-1, a compound of Formula I-5 can be prepared by coupling a compound of S-2 can be coupled with a compound of S-9 under click-chemistry conditions, wherein J¹⁰ is alkyne and J¹¹ is N3 or J¹⁰ is N3 and J¹¹ is alkyne, wherein one instance of X in Formula I-5 is triazole, and wherein a+b+1 equals to m1. The variables L¹⁰, J¹, J², J³, R^{A}, R^{B}, G¹, G³, G⁴, A, r, m1, p1, and p2 in Scheme C-1 include any of those described and preferred herein in connection with Formula I in any combinations, provided that one instance of X in Fomrula I-5 is triazole formed by the coupling of J¹⁰ and J¹¹. As shown in Scheme C-2, in some embodiments, a compound of Formula I-5 can be prepared by the acid of S-10 with an amine of S-6 under amide coupling conditions, which can then be followed by deprotection to provide the compound of Formula I-5, wherein Pg1 in S-10 refers to a carboxylic acid protecting group, such as a tert-butyl group. The variables L¹⁰, J¹, J², J³, R^{A}, R^{B}, G¹, G³, G⁴, A, r, m1, p1, and p2 in Scheme C-2 include any of those described and preferred herein in connection with Formula I in any combinations.

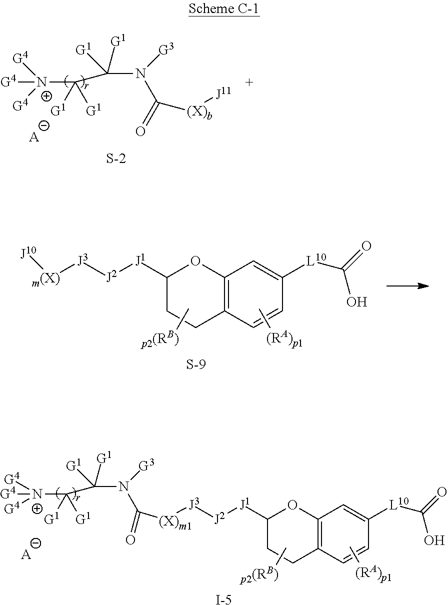

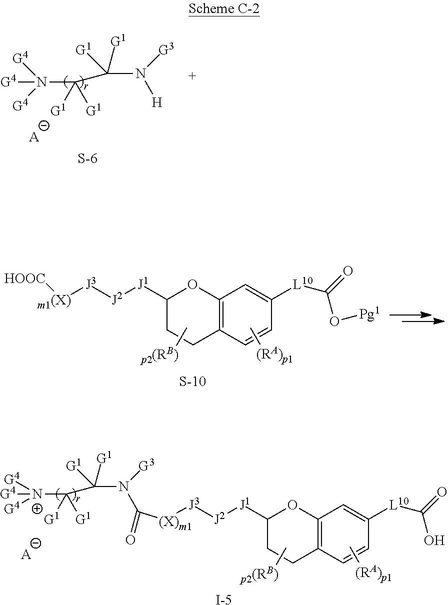

Other compounds of Formula I, II, II-B, III, or III-B can be prepared similarly to those shown in Schemes A-1, A-2, A-3, B-1, B-2, B-3, C-1, and C-2. Exemplified procedures are also shown in the Examples section herein.

In some embodiments, the present disclosure also provides synthetic intermediates and methods according to any of those described herein, such as those shown in Schemes A-1, A-2, A-3, B-1, B-2, B-3, C-1, and C-2 and those shown in the schemes in the Examples section. To be clear, the synthetic intermediates shown in the Examples section in a different salt form and/or having a different counterion(s) are within the scope of this disclosure. In some embodiments, the present disclosure provides a novel synthetic intermediate shown in the Examples section herein, which as applicable, may exist in any salt form and/or have a different counterion(s) as those shown in the Examples section herein.

As will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in “Protective Groups in Organic Synthesis”, 4th ed. P. G. M. Wuts; T. W. Greene, John Wiley, 2007, and references cited therein. The reagents for the reactions described herein are generally known compounds or can be prepared by known procedures or obvious modifications thereof. For example, many of the reagents are available from commercial suppliers such as Aldrich Chemical Co. (Milwaukee, Wisconsin, USA), Sigma (St. Louis, Missouri, USA). Others may be prepared by procedures, or obvious modifications thereof, described in standard reference texts such as

Fieser and Fieser's Reagents for Organic Synthesis, Volumes 1-15 (John Wiley and Sons, 1991), Rodd's Chemistry of Carbon Compounds, Volumes 1-5 and Supplemental (Elsevier Science Publishers, 1989), Organic Reactions, Volumes 1-40 (John Wiley and Sons, 1991), March's Advanced Organic Chemistry, (Wiley, 7th Edition), and Larock's Comprehensive Organic Transformations (Wiley-VCH, 1999), and any of available updates as of this filing.

### Pharmaceutical Compositions

Certain embodiments are directed to a pharmaceutical composition comprising one or more compounds of the present disclosure.

The pharmaceutical composition can optionally contain a pharmaceutically acceptable excipient. In some embodiments, the pharmaceutical composition comprises a compound of the present disclosure (e.g., a compound of Formula I (e.g., Formula I-1, I-2, I-3, I-4, I-5, I-6, I-7, I-8, I-9, I-1-A, I-4-A, I-4-B, I-4-C, I-5-A, I-5-B, I-5-C, I-1-A-1, I-1-A-2, I-1-A-3, I-1-A-4, I-1-A-5, I-1-A-6, I-1-A-7, I-1-A-8, I-1-A-9, or I-1-A-10), Formula II (e.g., Formula II-1, II-2, II-3, II-4, II-5, II-6, II-7, II-8, II-9, II-1-A, II-4-A, II-5-A, or II-1-A-1), Formula II-B (e.g., Formula II-B-1, II-B-2, or II-B-3), Formula III (e.g., Formula III-1, III-2, III-3, III-4, III-5, III-6, III-7, III-8, III-9, III-1-A, III-4-A, III-5-A, or III-1-A-1), Formula III-B (e.g., III-B-1, III-B-2, or III-B-3), or any of the compounds listed in Table 1 herein, any of the compounds according to Examples 1-221 herein, or a pharmaceutically acceptable salt or ester thereof) and a pharmaceutically acceptable excipient. Pharmaceutically acceptable excipients are known in the art. Non-limiting suitable excipients include, for example, encapsulating materials or additives such as antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. See also Remington's The Science and Practice of Pharmacy, 21st Edition, A. R. Gennaro (Lippincott, Williams & Wilkins, Baltimore, Md., 2005; incorporated herein by reference), which discloses various excipients used in formulating pharmaceutical compositions and known techniques for the preparation thereof.

The pharmaceutical composition can include any one or more of the compounds of the present disclosure. For example, in some embodiments, the pharmaceutical composition comprises a compound of Formula I (e.g., Formula I-1, I-2, I-3, I-4, I-5, I-6, I-7,

I-8, 1-9, I-1-A, I-4-A, I-4-B, I-4-C, I-5-A, I-5-B, I-5-C, I-1-A-1, I-1-A-2, I-1-A-3, I-1-A-4, I-1-A-5, I-1-A-6, I-1-A-7, I-1-A-8, I-1-A-9, or I-1-A-10), Formula II (e.g., Formula II-1, II-2, II-3, II-4, II-5, II-6, II-7, II-8, II-9, II-1-A, II-4-A, II-5-A, or II-1-A-1), Formula II-B (e.g., Formula II-B-1, II-B-2, or II-B-3), Formula III (e.g., Formula III-1, III-2, III-3, III-4, III-5, III-6, III-7, III-8, III-9, III-1-A, III-4-A, III-5-A, or III-1-A-1), Formula III-B (e.g., III-B-1, III-B-2, or III-B-3), or any of the compounds listed in Table 1 herein, any of the compounds according to Examples 1-221 herein, or a pharmaceutically acceptable salt or ester thereof, e.g., in a therapeutically effective amount. In any of the embodiments described herein, the pharmaceutical composition can comprise a therapeutically effective amount of a compound selected from the compounds listed in Table 1 herein, or a pharmaceutically acceptable salt or ester thereof. In any of the embodiments described herein, the pharmaceutical composition can comprise a therapeutically effective amount of a compound according to Examples 1-221 herein, or a pharmaceutically acceptable salt thereof.

In some embodiments, the pharmaceutical composition can be formulated for oral administration. Typically, the pharmaceutical composition is administered to a subject in need to deliver an effective amount of GPR40 agonist in the gastrointestinal tract with minimal or no absorption of GPR40 agonist in systemic circulation. The oral formulations can be presented in discrete units, such as capsules, pills, cachets, lozenges, or tablets, each containing a predetermined amount of the active compound; as a powder or granules; as a solution or a suspension in an aqueous or non-aqueous liquid; or as an oil-in-water or water-in-oil emulsion. Excipients for the preparation of compositions for oral administration are known in the art. Non-limiting suitable excipients include, for example, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl cellulose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, and mixtures thereof.

Compounds of the present disclosure can be used alone, in combination with each other, or in combination with one or more additional therapeutic agents, e.g., PPAR gamma agonists and partial agonists; biguanides; protein tyrosine phosphatase-1B (PTP-1B) inhibitors; dipeptidyl peptidase IV (DPP-IV) inhibitors; insulin or an insulin mimetic; sulfonylureas; a-glucosidase inhibitors; agents which improve a patient's lipid profile, said agents being selected from the group consisting of (i) HMG-COA reductase inhibitors, (ii) bile acid sequestrants, (iii) nicotinyl alcohol, nicotinic acid or a salt thereof, (iv) PPARa agonists, (v) cholesterol absorption inhibitors, (vi) acyl CoA: cholesterol acyltransferase (ACAT) inhibitors, (vii) CETP inhibitors, (viii) PCSK9 inhibitor or antibodies; (ix) apolipoproteins inhibitors; (x) phenolic anti-oxidants; PPARα/γ dual agonists; PPAR8 agonists; PPAR a/8 partial agonists; antiobesity compounds; ileal bile acid transporter inhibitors; anti-inflammatory agents; glucagon receptor antagonists; glucokinase activators; GLP-1 and GLP-1 analogs; GLP-1 receptor agonists (peptide and small-molecule); GLP-1/GIP receptor dual agonists; GLP-1/glucagon receptor dual agonists; GLP-1/GIP/insulin receptor triple agonists; GLP-1/GIP/glucagon receptor triple agonists; GIP receptor antibody; GLP-1 analog/GIP receptor antibody; PYY analog; amylin analogs; GPR119 agonist; TGR5 agonist; SSTR3 and/or SSTR5 antagonist or inverse agonist; THRβ agonists; HSD-1 inhibitors; HSD-17 inhibitors and degraders; PNPLA3 inhibitors and degraders; SGLT-2 inhibitors; SGLT-1/SGLT-2 inhibitors; enteric alpha-glucosidase inhibitors; FXR agonists; DGAT1 and/or DGAT2 inhibitors; FGF19 and analogs; FGF21 and analogs; GDF15 and analogs; ANGPTL3 antibody or inhibitor; ANGPTL3/8 antibody; ANGPTL4 inhibitor; Oxyntomodulin; (xi) anti-amyloid beta antibody; (xii) anti-inflammatory agents including but not limited to PDE4 inhibitors, JAK inhibitors, TYK2 inhibitors, S1P receptor modulators, NLRP3 inhibitors, BTK inhibitors, IRAK1 inhibitors, IRAK4 inhibitors, glucocorticoids, anti-TNFα antibodies, anti-IL-12/IL-23 antibodies, (xiii) anti-integrin antibodies including anti-a4ß7, anti-α4, anti-β7, anti-MAdCAM-1. These additional therapeutic agents are known in the art, some of which are exemplified in the background section. Additional example can be found in various patent literatures, for example, as described in U.S. Published

Application No. 20190367495, the content of which is herein incorporated by reference.

When used in combination with one or more additional therapeutic agents, compounds of the present disclosure or pharmaceutical compositions herein can be administered to the subject either concurrently or sequentially in any order with such additional therapeutic agents. In some embodiments, the pharmaceutical composition can comprise one or more compounds of the present disclosure and the one or more additional therapeutic agents in a single composition. In some embodiments, the pharmaceutical composition comprising one or more compounds of the present disclosure can be included in a kit which also comprises a separate pharmaceutical composition comprising the one or more additional therapeutic agents.

The pharmaceutical composition can include various amounts of the compounds of the present disclosure, depending on various factors such as the intended use and potency and selectivity of the compounds. In some embodiments, the pharmaceutical composition comprises a therapeutically effective amount of a compound of the present disclosure. In some embodiments, the pharmaceutical composition comprises a therapeutically effective amount of the compound of the present disclosure and a pharmaceutically acceptable excipient. As used herein, a therapeutically effective amount of a compound of the present disclosure is an amount effective to treat a disorder, condition or disease as described herein, such as type 2 diabetes, which can depend on the recipient of the treatment, the disorder, condition or disease being treated and the severity thereof, the composition containing the compound, the time of administration, the route of administration, the duration of treatment, the compound potency, its rate of clearance and whether or not another drug is co-administered.

### Method of Treatment/Use

Compounds of the present disclosure have various utilities. For example, compounds of the present disclosure can be used as therapeutic active substances for the treatment and/or prophylaxis of disorders, conditions or diseases that are associated with G-protein-coupled receptor 40 (“GPR40”). Accordingly, some embodiments of the present disclosure are also directed to methods of using one or more compounds of the present disclosure or pharmaceutical compositions herein for treating or preventing a disorder, condition or disease that may be responsive to the agonism of the G-protein-coupled receptor 40 (“GPR40”) in a subject in need thereof, such as for treating type 2 diabetes mellitus in a subject in need thereof.

In some embodiments, the present disclosure provides a method of treating or preventing a disorder, condition or disease that may be responsive to the agonism of the G-protein-coupled receptor 40 (“GPR40”) in a subject in need thereof. In some embodiments, the method comprises administering an effective amount of a compound of the present disclosure (e.g., a compound of Formula I (e.g., Formula I-1, I-2, 1-3, I-4, I-5, I-6, 1-7, I-8, I-9, I-1-A, I-4-A, I-4-B, I-4-C, I-5-A, I-5-B, I-5-C, I-1-A-1, I-1-A-2, I-1-A-3, I-1-A-4, I-1-A-5, I-1-A-6, I-1-A-7, I-1-A-8, I-1-A-9, or I-1-A-10), Formula II (e.g., Formula II-1, II-2, II-3, II-4, II-5, II-6, II-7, II-8, II-9, II-1-A, II-4-A, II-5-A, or II-1-A-1), Formula II-B (e.g., Formula II-B-1, II-B-2, or II-B-3), Formula III (e.g., Formula III-1, III-2, III-3, III-4, III-5, III-6, III-7, III-8, III-9, III-1-A, III-4-A, III-5-A, or III-1-A-1), Formula III-B (e.g., III-B-1, III-B-2, or III-B-3), or any of the compounds listed in Table 1 herein, any of the compounds according to Examples 1-221 herein, or a pharmaceutically acceptable salt or ester thereof) or an effective amount of a pharmaceutical composition described herein. In some embodiments, the disorder, condition or disease that may be responsive to agonism of GPR40 is Type 1 or 2 diabetes, obesity, hyperglycemia, glucose intolerance, insulin resistance, hyperinsulinemia, hypercholesterolemia, hypertension, hyperlipoproteinemia, hyperlipidemia, myocardial infarction, stroke, hypertriglylceridemia, dyslipidemia, metabolic syndrome, syndrome X, cardiovascular disease, atherosclerosis, kidney disease, diabetic kidney disease, ketoacidosis, thrombotic disorders, nephropathy, diabetic neuropathy, diabetic retinopathy, sexual dysfunction, dermatopathy, dyspepsia, hypoglycemia, cancer, edema, nonalcoholic steatohepatitis (NASH), lipodystrophy, Prader Willi syndrome, inflammatory bowel diseases including Crohn's disease and ulcerative colitis, irritable bowel syndrome, short bowel syndrome, lymphocytic colitis, rare microscopic colitis, and/or neurodegenerative diseases including but not limited to Alzheimer's disease, Parkinson's disease, amyotrophic lateral sclerosis.

In some embodiments, the present disclosure also provides a method of treating type 2 diabetes mellitus in a subject in need thereof. In some embodiments, the method comprises administering an effective amount of a compound of the present disclosure (e.g., a compound of Formula I (e.g., Formula I-1, I-2, I-3, I-4, I-5, I-6, I-7, I-8, I-9, I-1-A, I-4-A, I-4-B, I-4-C, I-5-A, I-5-B, I-5-C, I-1-A-1, I-1-A-2, I-1-A-3, I-1-A-4, I-1-A-5, I-1-A-6, I-1-A-7, I-1-A-8, I-1-A-9, or I-1-A-10), Formula II (e.g., Formula II-1, II-2, II-3, II-4, II-5, II-6, II-7, II-8, II-9, II-1-A, II-4-A, II-5-A, or II-1-A-1), Formula II-B (e.g., Formula II-B-1, II-B-2, or II-B-3), Formula III (e.g., Formula III-1, III-2, III-3, III-4, III-5, III-6, III-7, III-8, III-9, III-1-A, III-4-A, III-5-A, or III-1-A-1), Formula III-B (e.g., III-B-1, III-B-2, or III-B-3), or any of the compounds listed in Table 1 herein, any of the compounds according to Examples 1-221 herein, or a pharmaceutically acceptable salt or ester thereof) or an effective amount of a pharmaceutical composition described herein.

The administering in the methods herein is not limited. In some embodiments, the administering is orally.

As discussed herein, compounds of the present disclosure can be used as a monotherapy or in a combination therapy. In some embodiments according to the methods described herein, compounds of the present disclosure can be administered as the only active ingredient(s).

In some embodiments according to the methods described herein, compounds of the present disclosure can also be co-administered with an additional therapeutic agent, either concurrently or sequentially in any order, to the subject in need thereof. In some embodiments, the additional therapeutic agent can be PPAR gamma agonists and partial agonists; biguanides; protein tyrosine phosphatase-1B (PTP-1B) inhibitors; dipeptidyl peptidase IV (DPP-IV) inhibitors; insulin or an insulin mimetic; sulfonylureas; a-glucosidase inhibitors; agents which improve a patient's lipid profile, said agents being selected from the group consisting of (i) HMG-COA reductase inhibitors, (ii) bile acid sequestrants, (iii) nicotinyl alcohol, nicotinic acid or a salt thereof, (iv) PPARa agonists, (v) cholesterol absorption inhibitors, (vi) acyl CoA: cholesterol acyltransferase (ACAT) inhibitors, (vii) CETP inhibitors, (viii) PCSK9 inhibitor or antibodies; (ix) apolipoproteins inhibitors; (x) phenolic anti-oxidants; PPARa/y dual agonists; PPAR8 agonists; PPAR a/8 partial agonists; antiobesity compounds; ileal bile acid transporter inhibitors; anti-inflammatory agents; glucagon receptor antagonists; glucokinase activators; GLP-1 and GLP-1 analogs; GLP-1 receptor agonists (peptide and small-molecule); GLP-1/GIP receptor dual agonists; GLP-1/glucagon receptor dual agonists; GLP-1/GIP/insulin receptor triple agonists; GLP-1/GIP/glucagon receptor triple agonists; GIP receptor antibody; GLP-1 analog/GIP receptor antibody; PYY analog; amylin analogs; GPR119 agonist; TGR5 agonist; SSTR3 and/or SSTR5 antagonist or inverse agonist; THRβ agonists; HSD-1 inhibitors; HSD-17 inhibitors and degraders; PNPLA3 inhibitors and degraders; SGLT-2 inhibitors; SGLT-1/SGLT-2 inhibitors; enteric alpha-glucosidase inhibitors; FXR agonists; DGAT1 and/or DGAT2 inhibitors; FGF19 and analogs; FGF21 and analogs; GDF15 and analogs; ANGPTL3 antibody or inhibitor; ANGPTL3/8 antibody; ANGPTL4 inhibitor; Oxyntomodulin; (xi) anti-amyloid beta antibody; (xii) anti-inflammatory agents including but not limited to PDE4 inhibitors, JAK inhibitors, TYK2 inhibitors, S1P receptor modulators, NLRP3 inhibitors, BTK inhibitors, IRAK1 inhibitors, IRAK4 inhibitors, glucocorticoids, anti-TNFα antibodies, anti-IL-12/IL-23 antibodies, (xiii) anti-integrin antibodies including anti-α4ß7, anti-α4, anti-β7, anti-MAdCAM-1.

Dosing regimen including doses for the methods described herein can vary and be adjusted, which can depend on the recipient of the treatment, the disorder, condition or disease being treated and the severity thereof, the composition containing the compound, the time of administration, the route of administration, the duration of treatment, the compound potency, its rate of clearance and whether or not another drug is co-administered.

### Definitions

It is meant to be understood that proper valences are maintained for all moieties and combinations thereof.

When a variable or structure herein defined as containing a charged group, such as those containing a quaternary nitrogen atom, it should be understood that the compound containing such variable or structure is overall neutral; in other words, any charge associated with the variable or structure is balanced with a counterion as necessary to maintain the compound's overall electronic neutrality, whether or not the counterion is explicitly drawn or described. Suitable counterions are not particularly limited, however, preferably, the counterion herein is a pharmaceutically acceptable counterion, such as a pharmaceutically acceptable anion, which may be monovalent (e.g., including one formal negative charge) or multivalent (e.g., including more than one formal negative charge), such as divalent or trivalent. Non-limiting exemplary suitable counterions include halide ions (e.g., F, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan −1-sulfonic acid-2-sulfonate, and the like), carboxylate ions (e.g., acetate, propanoate, benzoate, glycerate, lactate, tartrate, glycolate, gluconate, tartrate, citrate, fumarate, maleate, malate, malonate, gluconate, succinate, glutarate, adipate, pimelate, suberate, azelate, sebacate, Salicylate, phthalates, aspartate, glutamate, and the like), BF₄⁻, PF₄³¹, PF₆⁻, AsF₆⁻, SbF₆⁻, B[3,5-(CF₃)₂C₆H₃]₄]⁻, BPh₄⁻, Al(OC(CF₃)₃)₄⁻, carborane anions (e.g., CB₁₁H₁₂⁻ or (HCB₁₁Me₅Br₆))⁻), CO₃²⁻, HPO₄²⁻, PO₄³⁻, B₄O₇²⁻, SO₄²⁻, S₂O₃²⁻, etc.

It is also meant to be understood that a specific embodiment of a variable moiety herein can be the same or different as another specific embodiment having the same identifier.

Suitable groups for in compounds of Formula I, II, II-B, III, III-B, GPR-1, GPR-2, GPR-2B, GPR-3, or GPR-3B, or subformula thereof, as applicable, are independently selected. The described embodiments of the present disclosure can be combined. Such combination is contemplated and within the scope of the present disclosure. For example, it is contemplated that the definition(s) of any one or more of q, T^{A}, L^{A}, L¹⁰, J¹, J², J³, R^{A}, R^{B}, p1, and p2 of Formula I can be combined with the definition of any one or more of the other(s) of q, T^{A}, L^{A}, L¹⁰, J¹, J², J³, R^{A}, R^{B}, p1, and p2 as applicable, and the resulted compounds from the combination are within the scope of the present disclosure. Combinations of other variables for other Formulae should be understood similarly. To be clear, it should be understood that with respect to any formula herein, unless specified or contrary from context, the definition and preferred definition of a variable appearing in a formula can be any of those respective definition and preferred definition shown herein for the variable in connection with a parent formula (or any of the sub-formulae of the parent formula) or any other formula that is indicated as applicable. For example, unless specified or contrary from context, a variable appearing in Formula I-1-A can have a definition as defined for the variable in connection with Formula I or any of its other sub-formulae (e.g., I-1, I-2, etc.). As a further example, unless specified or contrary from context, the definition and preferred definition of L^{A} and/or T^{A} in connection with any formula herein is generally applicable to all other formulae herein.

The symbol, ˜, displayed perpendicular to (or otherwise crossing) a bond, indicates the point at which the displayed moiety is attached to the remainder of the molecule. It should be noted that in some chemical drawings herein, the immediately connected group or groups are shown beyond the symbol, ˜˜, to indicate direction of attachment, as would be understood by those skilled in the art.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high performance liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers including racemic mixtures. When a stereochemistry is specifically drawn, unless otherwise contradictory from context, it should be understood that with respect to that particular chiral center or axial chirality, the compound can exist predominantly as the as -drawn stereoisomer, such as with less than 20%, less than 10%, less than 5%, less than 1%, by weight, by HPLC area, or both, or with a non-detectable amount of the other stereoisomer(s). In some preferred embodiments, the compound herein can exist predominantly as the as -drawn stereoisomer, with an enantiomeric excess (“ee”) of at least 70%, for example, with an ee of at least 80%, at least 85%, at least 90%, at least 95%, at least 98%, or at least 99%, or the other enantiomer is non-detectable. The presence and/or amounts of stereoisomers can be determined by those skilled in the art in view of the present disclosure, including through the use of chiral HPLC or other methods.

When a range of values is listed, it is intended to encompass each value and sub-range within the range. For example “C₁₋₆” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

As used herein, the term “compound(s) of the present disclosure” refers to any of the compounds described herein according to Formula I (e.g., Formula I-1, I-2, I-3, I-4, I-5, I-6, I-7, I-8, I-9, I-1-A, I-4-A, I-4-B, I-4-C, I-5-A, I-5-B, I-5-C, I-1-A-1, I-1-A-2, I-1-A-3, I-1-A-4, I-1-A-5, I-1-A-6, I-1-A-7, I-1-A-8, I-1-A-9, or I-1-A-10), Formula II (e.g., Formula II-1, II-2, II-3, II-4, II-5, II-6, II-7, II-8, II-9, II-1-A, II-4-A, II-5-A, or II-1-A-1), Formula II-B (e.g., Formula II-B-1, II-B-2, or II-B-3), Formula III (e.g., Formula III-1, III-2, III-3, III-4, III-5, III-6, III-7, III-8, III-9, III-1-A, III-4-A, III-5-A, or III-1-A-1), Formula III-B (e.g., III-B-1, III-B-2, or III-B-3), or any of the compounds listed in Table 1 herein, any of the compound according to Examples 1-221 herein, GPR-1, GPR-2, GPR-2B, GPR-3, or GPR-3B, isotopically labeled compound(s) thereof (such as a deuterated analog wherein at least one of the hydrogen atoms is substituted with a deuterium atom with an abundance above its natural abundance), possible regioisomers, possible stereoisomers thereof (including diastereoisomers, enantiomers, and racemic mixtures), tautomers thereof, conformational isomers thereof, pharmaceutically acceptable esters thereof, a zwitterion structure thereof, and/or possible pharmaceutically acceptable salts thereof (e.g., acid addition salt such as HCl salt or base addition salt such as Na salt). Hydrates and solvates of the compounds of the present disclosure are considered compositions of the present disclosure, wherein the compound(s) is in association with water or solvent, respectively. To be clear, as used herein, a compound according to Examples 1-221 herein or a pharmaceutically acceptable salt thereof should be understood as encompassing any compound, or pharmaceutically acceptable salt thereof, having the structure of any of Examples 1-221 as shown in the Examples section herein, except that the counterion and/or salt form may be different. For example, a compound according to Example 1 or a pharmaceutically acceptable salt thereof should be understood as encompassing a base form of Example 1, a pharmaceutically acceptable salt thereof, which is not limited to the 4HCl addition salt, or any combinations thereof. Similarly, a compound according to Example 2 or a pharmaceutically acceptable salt thereof should be understood as encompassing a base form of Example 2 with a counterion for the quaternary nitrogen being Cl⁻ or any other pharmaceutically acceptable counterion or an internal counterion, a pharmaceutically acceptable salt thereof, which is not limited to the salt form of 3HCl addition salt and a counterion of Cl⁻ for the quaternary nitrogen, or any combinations thereof.

Compounds of the present disclosure can exist in isotope-labeled or -enriched form containing one or more atoms having an atomic mass or mass number different from the atomic mass or mass number most abundantly found in nature. Isotopes can be radioactive or non-radioactive isotopes. Isotopes of atoms such as hydrogen, carbon, phosphorous, sulfur, fluorine, chlorine, and iodine include, but are not limited to ²H, ³H, ¹³C, ¹⁴C, ¹⁵N, ¹⁸O, ³²P, ³⁵S, ¹⁸F, ³⁶Cl, and ¹²⁵I. Compounds that contain other isotopes of these and/or other atoms are within the scope of this invention.

As used herein, the phrase “administration” of a compound, “administering” a compound, or other variants thereof means providing the compound or a prodrug of the compound to the individual in need of treatment.

As used herein, the term “alkyl” as used by itself or as part of another group refers to a straight- or branched-chain aliphatic saturated hydrocarbon. In some embodiments, the alkyl which can include one to twelve carbon atoms (i.e., C₁₋₁₂ alkyl) or the number of carbon atoms designated. In one embodiment, the alkyl group is a straight chain C₁₋₁₀ alkyl group. In another embodiment, the alkyl group is a branched chain C₃₋₁₀ alkyl group. In another embodiment, the alkyl group is a straight chain C₁₋₆ alkyl group. In another embodiment, the alkyl group is a branched chain C₃₋₆ alkyl group. In another embodiment, the alkyl group is a straight chain C₁₋₄ alkyl group. For example, a C₁₋₄ alkyl group includes methyl, ethyl, propyl(n-propyl), isopropyl, butyl(n-butyl), sec-butyl, tert-butyl, and iso-butyl. As used herein, the term “alkylene” as used by itself or as part of another group refers to a divalent radical derived from an alkyl group. For example, non-limiting straight chain alkylene groups include-CH₂—CH₂—CH₂—CH₂—, —CH₂—CH₂—CH₂—, —CH₂—CH₂—, and the like.

As used herein, the term “alkenyl” as used by itself or as part of another group refers to a straight- or branched-chain aliphatic hydrocarbon containing one or more, for example, one, two or three carbon-to-carbon double bonds. In one embodiment, the alkenyl group is a C₂₋₆ alkenyl group. In another embodiment, the alkenyl group is a C₂₋₄ alkenyl group. Non-limiting exemplary alkenyl groups include ethenyl, propenyl, isopropenyl, butenyl, sec-butenyl, pentenyl, and hexenyl.

As used herein, the term “alkynyl” as used by itself or as part of another group refers to a straight- or branched-chain aliphatic hydrocarbon containing one or more, for example, one to three carbon-to-carbon triple bonds. In one embodiment, the alkynyl has one carbon-carbon triple bond. In one embodiment, the alkynyl group is a C₂₋₆ alkynyl group. In another embodiment, the alkynyl group is a C₂₋₄ alkynyl group. Non-limiting exemplary alkynyl groups include ethynyl, propynyl, butynyl, 2-butynyl, pentynyl, and hexynyl groups.

As used herein, the term “alkoxy” as used by itself or as part of another group refers to a radical of the formula OR^{a1}, wherein R^{a1} is an alkyl.

As used herein, the term “cycloalkoxy” as used by itself or as part of another group refers to a radical of the formula OR^{a1}, wherein R^{a1} is a cycloalkyl.

As used herein, the term “haloalkyl” as used by itself or as part of another group refers to an alkyl substituted with one or more fluorine, chlorine, bromine and/or iodine atoms. In preferred embodiments, the haloalkyl is an alkyl group substituted with one, two, or three fluorine atoms. In one embodiment, the haloalkyl group is a C₁₋₁₀ haloalkyl group. In one embodiment, the haloalkyl group is a C₁₋₆ alkyl haloalkyl group. In one embodiment, the haloalkyl group is a C₁₋₄ haloalkyl group.

As used herein, the term “heteroalkyl,” by itself or in combination with another term, means, unless otherwise stated, a stable straight or branched-chain alkyl group, e.g., having from 2 to 14 carbons, such as 2 to 10 carbons in the chain, one or more of which has been replaced by a heteroatom selected from S, O, P and N, and wherein the nitrogen, phosphine, and sulfur atoms can optionally be oxidized and the nitrogen heteroatom can optionally be quaternized. The heteroatom(s) S, O, P and N may be placed at any interior position of the heteroalkyl group or at the position at which the alkyl group is attached to the remainder of the molecule. For example, C₁₋₄ heteroalkyl include but not limited to, C₄ heteroalkyl such as —CH₂—CH₂—N(CH₃)—CH₃, C₃ heteroalkyl such as —CH₂—CH₂—O—CH₃, —CH₂—CH₂—NH—CH₃, —CH₂—S—CH₂—CH₃, —CH₂—CH₂—S(O)—CH₃, and —CH₂—CH₂—S(O)₂—CH₃, C₂ heteroalkyl such as —O—CH₂—CH₃ and C₁ heteroalkyl such as O—CH₃, etc. To be clear, when the heteroalkyl is referred to as xx-membered, the number of carbon and heteroatoms forming the heteroalkyl should be counted together, but not the potential oxidation, for example, sulfur oxide or N-oxide is counted as one member. For example, —CH₂—CH₂—N(CH₃)—CH₃ or —CH₂—[N(CH₃)₃]⁺ may be considered a five-membered heteroalkyl. Additionally, as an example, a four-membered heteroalkyl includes-CH₂—CH₂—O—CH₃, —CH₂—CH₂—NH—CH₃, —CH₂—S—CH₂—CH₃, —CH₂—CH₂—S(O)—CH₃, and —CH₂—CH₂—S(O)₂—CH₃, a three-membered heteroalkyl includes-O—CH₂—CH₃, and a two-membered heteroalkyl includes O—CH₃.

Similarly, for the purposes herein, when counting the number of heteroatoms in a heteroalkyl group, the oxygen atom from potential oxidation is not counted, thus, —CH₂—S—CH₂—CH₃, —CH₂—CH₂—S(O)—CH₃, and —CH₂—CH₂—S(O)₂—CH₃ should all be considered as a 4-membered, C₃ heteroalkyl having one heteroatom, S, in which the S is optionally oxidized. Similarly, the term “heteroalkylene” by itself or as part of another substituent means a divalent radical derived from heteroalkyl, as exemplified, but not limited by, —CH₂—CH₂—O—CH₂—CH₂— and -O—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. Where “heteroalkyl” is recited, followed by recitations of specific heteroalkyl groups, such as —NR′R″ or the like, it will be understood that the terms heteroalkyl and -NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Carbocyclyl” or “carbocyclic” as used by itself or as part of another group refers to a radical of a non-aromatic cyclic hydrocarbon group having at least 3 carbon atoms, e.g., from 3 to 10 ring carbon atoms (“C₃₋₁₀ carbocyclyl”), and zero heteroatoms in the non-aromatic ring system. The carbocyclyl group can be either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclic ring, as defined above, is fused with one or more aryl or heteroaryl groups wherein the point of attachment is on the carbocyclic ring, and in such instances, the number of carbons continue to designate the number of carbons in the carbocyclic ring system. Non-limiting exemplary carbocyclyl groups include cyclopropyl, cyclobutyl, cyclopentyl, cyclohexyl, cycloheptyl, cyclooctyl, norbornyl, decalin, adamantyl, cyclopentenyl, and cyclohexenyl. As used herein, the term “carbocyclylene” as used by itself or as part of another group refers to a divalent radical derived from the carbocyclyl group defined herein.

In some embodiments, “carbocyclyl” is fully saturated, which is also referred to as cycloalkyl. In some embodiments, the cycloalkyl can have from 3 to 10 ring carbon atoms (“C₃₋₁₀ cycloalkyl”). In preferred embodiments, the cycloalkyl is a monocyclic ring. As used herein, the term “cycloalkylene” as used by itself or as part of another group refers to a divalent radical derived from a cycloalkyl group, for example,

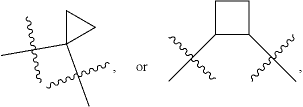 [SMILES: *C1(*)CC1 ; *C1CCC1*]

etc.

“Heterocyclyl” or “heterocyclic” as used by itself or as part of another group refers to a radical of a 3-membered or greater, such as 3- to 14-membered, non-aromatic ring system having ring carbon atoms and at least one ring heteroatom, such as 1 to 4 ring heteroatoms, wherein each heteroatom is independently selected from nitrogen, oxygen, sulfur, boron, phosphorus, and silicon. In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system, such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclic ring, as defined above, is fused with one or more carbocyclyl groups wherein the point of attachment is on the heterocyclic ring, or ring systems wherein the heterocyclic ring, as defined above, is fused with one or more aryl or heteroaryl groups, wherein the point of attachment is on the heterocyclic ring, and in such instances, the number of ring members continue to designate the number of ring members in the heterocyclic ring system. As used herein, the term “heterocyclylene” as used by itself or as part of another group refers to a divalent radical derived from the heterocyclyl group defined herein. For example, a piperidinylene group includes two attaching points from the piperidine ring:

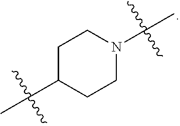 [SMILES: *C1CCN(*)CC1]

The heterocyclyl or heterocylylene can be optionally linked to the rest of the molecule through a carbon or nitrogen atom.

Exemplary 3-membered heterocyclyl groups containing one heteroatom include, without limitation, azirdinyl, oxiranyl, thiiranyl. Exemplary 4 membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5 dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Aryl” as used by itself or as part of another group refers to a radical of a monocyclic or polycyclic (e.g., bicyclic or tricyclic)4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. As used herein, the term “arylene” as used by itself or as part of another group refers to a divalent radical derived from the aryl group defined herein. For example, a phenylene group includes two attaching points from the benzene ring, for example, 1,3-phenylene, 1,4-phenylene:

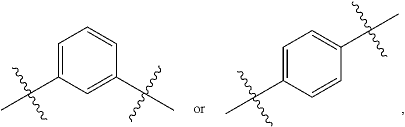 [SMILES: *c1cccc(*)c1 ; *c1ccc(*)cc1]

etc.

“Aralkyl” as used by itself or as part of another group refers to an alkyl substituted with one or more aryl groups, preferably, substituted with one aryl group. Examples of aralkyl include benzyl, phenethyl, etc. When an aralkyl is said to be optionally substituted, either the alkyl portion or the aryl portion of the aralkyl can be optionally substituted.

“Heteroaryl” as used by itself or as part of another group refers to a radical of a 5-14 membered monocyclic, bicyclic, or tricyclic 4n+2 aromatic ring system (e.g., having 6 or 10 pi electrons shared in a cyclic array) having ring carbon atoms and at least one, preferably, 1-4, ring heteroatoms provided in the aromatic ring system, wherein each heteroatom is independently selected from nitrogen, oxygen and sulfur (“5-14 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, and the like) the point of attachment can be on either ring, i.e., either the ring bearing a heteroatom (e.g., 2-indolyl) or the ring that does not contain a heteroatom (e.g., 5-indolyl). As used herein, the term “heteroarylene” as used by itself or as part of another group refers to a divalent radical derived from the heteroaryl group defined herein. For example, a pyridinylene group includes two attaching points from the pyridine ring, for example, 2,4-pyridinylene, 2,5-pyridinylene:

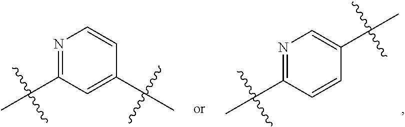 [SMILES: *c1ccnc(*)c1 ; *c1ccc(*)nc1]

etc.

Exemplary 5-membered heteroaryl groups containing one heteroatom include, without limitation, pyrrolyl, furanyl, and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

“Heteroaralkyl” as used by itself or as part of another group refers to an alkyl substituted with one or more heteroaryl groups, preferably, substituted with one heteroaryl group. When a heteroaralkyl is said to be optionally substituted, either the alkyl portion or the heteroaryl portion of the heteroaralkyl can be optionally substituted.

An “optionally substituted” group, such as an optionally substituted alkyl, optionally substituted alkenyl, optionally substituted alkynyl, optionally substituted carbocyclyl, optionally substituted heterocyclyl, optionally substituted aryl, and optionally substituted heteroaryl groups, refers to the respective group that is unsubstituted or substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent can be the same or different at each position. Typically, when substituted, the optionally substituted groups herein can be substituted with 1-5 substituents. Substituents can be a carbon atom substituent, a nitrogen atom substituent, an oxygen atom substituent or a sulfur atom substituent, as applicable. Two of the optional substituents can join to form an optionally substituted cycloalkyl, heterocylyl, aryl, or heteroaryl ring. Substitution can occur on any available carbon, oxygen, or nitrogen atom, and can form a spirocycle. Typically, substitution herein does not result in an O—O, O—N, S—S, S—N(except SO₂—N bond), heteroatom-halogen, or —C(O)—S bond or three or more consecutive heteroatoms, with the exception of O—SO₂—O, O—SO₂—N, and N—SO₂—N, except that some of such bonds or connections may be allowed if in a stable aromatic system.

In a broad aspect, the permissible substituents herein include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a cycloalkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, an aryl, or a heteroaryl, each of which can be substituted, if appropriate.

Exemplary substituents include, but not limited to, alkyl, alkenyl, alkynyl, aryl, heteroaryl, -alkylene-aryl, -arylene-alkyl, -alkylene-heteroaryl, -alkenylene-heteroaryl, -alkynylene-heteroaryl, —OH, hydroxyalkyl, haloalkyl, —O-alkyl, —O-haloalkyl, -alkylene-O-alkyl, —O-aryl, —O-alkylene-aryl, acyl, —C(O)-aryl, halo, —NO₂, —CN, —SF₅, —C(O)OH, —C(O)O-alkyl, —C(O)O-aryl, —C(O)O-alkylene-aryl, —S(O)-alkyl, —S(O)₂-alkyl, —S(O)-aryl, —S(O)₂-aryl, —S(O)-heteroaryl, —S(O)₂-heteroaryl, —S-alkyl, —S-aryl, —S-heteroaryl, —S-alkylene-aryl, —S-alkylene-heteroaryl, —S(O)₂-alkylene-aryl, —S(O)₂-alkylene-heteroaryl, cycloalkyl, heterocycloalkyl, —O C(O)-alkyl, —O—C(O)-aryl, —O—C(O)-cycloalkyl, C(—N—CN)—NH₂, —C(—NH)—NH₂, C(NH)—NH(alkyl), —N(Y₁)(Y₂), -alkylene-N(Y₁)(Y₂), —C(O) N(Y₁)(Y₂) and -S(O)₂N(Y₁)(Y₂), wherein Y₁ and Y₂ can be the same or different and are independently selected from the group consisting of hydrogen, alkyl, aryl, cycloalkyl, and -alkylene-aryl.

Some examples of suitable substituents include, but not limited to, (C₁-C₈)alkyl groups, (C₂-C₈)alkenyl groups, (C₂-C₈)alkynyl groups, (C₃-C₁₀) cycloalkyl groups, halogen (F, Cl, Br or I), halogenated (C₁-C₈)alkyl groups (for example but not limited to —CF₃), —O—(C₁-C₈)alkyl groups, —OH, —S—(C₁-C₈)alkyl groups, —SH, —NH(C₁-C₈)alkyl groups, —N((C₁-C₈)alkyl)₂ groups, —NH₂, —C(O)NH₂, —C(O)NH(C₁-C₈)alkyl groups, —C(O) N((C₁-C₈)alkyl)₂, —NHC(O) H, —NHC(O)(C₁-C₈)alkyl groups, —NHC(O)(C₃-C₈) cycloalkyl groups, —N((C₁-C₅)alkyl) C(O) H, —N((C₁-C₈)alkyl) C(O)(C₁-C₅)alkyl groups, —NHC(O)NH₂, —NHC(O)NH(C₁-C₈)alkyl groups, —N((C₁-C₈)alkyl) C(O)NH₂ groups, —NHC(O) N((C₁-C₅)alkyl)₂ groups, —N((C₁-C₈)alkyl) C(O) N((C₁-C₅)alkyl)₂ groups, —N((C₁-C₈)alkyl) C(O)NH((C₁-C₈)alkyl), —C(O) H, —C(O)(C₁-C₈)alkyl groups, —CN, —NO₂, —S (O)(C₁-C₈)alkyl groups, —S(O)₂ (C₁-C₅)alkyl groups, —S(O)₂N((C₁-C₈)alkyl)₂ groups, —S (O)₂NH(C₁-C₈)alkyl groups, —S(O)₂NH(C₃-C₈) cycloalkyl groups, —S(O)₂NH₂ groups, —NHS (O)₂ (C₁-C₈)alkyl groups, —N((C₁-C₈)alkyl) S (O)₂ (C₁-C₈)alkyl groups, —(C₁-C₈)alkyl-O—(C₁-C₈)alkyl groups, —O—(C₁-C₈)alkyl-O—(C₁-C₅)alkyl groups, —C(O)OH, —C(O) O (C₁-C₅)alkyl groups, NHOH, NHO (C₁-C₈)alkyl groups, —O-halogenated (C₁-C₈)alkyl groups (for example but not limited to —OCF₃), —S(O)₂-halogenated (C₁-C₈)alkyl groups (for example but not limited to —S(O)₂CF₃), —S-halogenated (C₁-C₅)alkyl groups (for example but not limited to —SCF₃), —(C₁-C₆) heterocycle (for example but not limited to pyrrolidine, tetrahydrofuran, pyran or morpholine), —(C₁-C₆) heteroaryl (for example but not limited to tetrazole, imidazole, furan, pyrazine or pyrazole), -phenyl, —NHC(O)O(C₁-C₆)alkyl groups, —N((C₁-C₆)alkyl) C(O)O—(C₁-C₆)alkyl groups, —C(═NH)—(C₁-C₆)alkyl groups, —C(═NOH)—(C₁-C₆)alkyl groups, or —C(═N—O—(C₁-C₆)alkyl)-(C₁-C₆)alkyl groups.

Exemplary carbon atom substituents include, but are not limited to, halogen, —CN, —NO₂, —N₃, hydroxyl, alkoxy, cycloalkoxy, aryloxy, amino, monoalkyl amino, dialkyl amino, amide, sulfonamide, thiol, acyl, carboxylic acid, ester, sulfone, sulfoxide, alkyl, haloalkyl, alkenyl, alkynyl, C₃₋₁₀ carbocyclyl, C₆₋₁₀ aryl, 3-10 membered heterocyclyl, 5-10 membered heteroaryl, etc. For example, exemplary carbon atom substituents can include F, Cl, —CN, —SO₂H, —SO₃H, —OH, —OC₁₋₆ alkyl, —NH₂, —N(C₁₋₆ alkyl)₂, —NH(C₁₋₆ alkyl), —SH, —SC₁₋₆ alkyl, —C(═O)(C₁₋₆ alkyl), —CO₂H, —CO₂ (C₁₋₆ alkyl), —OC (═O)(C₁₋₆ alkyl), —OCO₂ (C₁₋₆ alkyl), —C(═O) NH₂, —C(═O) N(C₁₋₆ alkyl)₂, —OC(═O) NH(C₁₋₆ alkyl), —NHC(═O)(C₁₋₆ alkyl), —N(C₁₋₆ alkyl) C(═O)(C₁₋₆ alkyl), —NHCO₂ (C₁₋₆ alkyl), —NHC(═O) N(C₁₋₆ alkyl)₂, —NHC (═O)NH(C₁₋₆ alkyl), —NHC(═O) NH₂, —NHSO₂ (C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂₀C₁₋₆ alkyl, —OSO₂C₁₋₆ alkyl, —SOC₁₋₆ alkyl, C₁₋₆ alkyl, C₁₋₆ alkyl haloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal substituents can be joined to form ═O.

Nitrogen atoms can be substituted or unsubstituted as valency permits, and include primary, secondary, tertiary, and quaternary nitrogen atoms. Exemplary nitrogen atom substituents include, but are not limited to, hydrogen, acyl groups, esters, sulfone, sulfoxide, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two substituent groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl can be further substituted as defined herein. In certain embodiments, the substituent present on a nitrogen atom is a nitrogen protecting group (also referred to as an amino protecting group). Nitrogen protecting groups are well known in the art and include those described in detail in Protective Groups in Organic Synthesis, T. W. Greene and P. G. M. Wuts, 3^{rd} edition, John Wiley & Sons, 1999, incorporated by reference herein. Exemplary nitrogen protecting groups include, but not limited to, those forming carbamates, such as Carbobenzyloxy (Cbz) group, p-Methoxybenzyl carbonyl(Moz or MeOZ) group, tert-Butyloxycarbonyl (BOC) group, Troc, 9-Fluorenylmethyloxycarbonyl (Fmoc) group, etc., those forming an amide, such as acetyl, benzoyl, etc., those forming a benzylic amine, such as benzyl, p-methoxybenzyl, 3,4-dimethoxybenzyl, etc., those forming a sulfonamide, such as tosyl, Nosyl, etc., and others such as p-methoxyphenyl.

Exemplary oxygen atom substituents include, but are not limited to, acyl groups, esters, sulfonates, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl can be further substituted as defined herein. In certain embodiments, the oxygen atom substituent present on an oxygen atom is an oxygen protecting group (also referred to as a hydroxyl protecting group). Oxygen protecting groups are well known in the art and include those described in detail in Protective Groups in Organic Synthesis, T. W. Greene and P. G. M. Wuts, 3^{rd} edition, John Wiley & Sons, 1999, incorporated herein by reference. Exemplary oxygen protecting groups include, but are not limited to, those forming alkyl ethers or substituted alkyl ethers, such as methyl, allyl, benzyl, substituted benzyls such as 4-methoxybenzyl, methoxylmethyl (MOM), benzyloxymethyl (BOM), 2-methoxyethoxymethyl (MEM), etc., those forming silyl ethers, such as trymethylsilyl (TMS), triethylsilyl (TES), triisopropylsilyl (TIPS), t-butyldimethylsilyl (TBDMS), etc., those forming acetals or ketals, such as tetrahydropyranyl (THP), those forming esters such as formate, acetate, chloroacetate, dichloroacetate, trichloroacetate, trifluoroacetate, methoxyacetate, etc., those forming carbonates or sulfonates such as methanesulfonate (mesylate), benzylsulfonate, and tosylate (Ts), etc.

Unless expressly stated to the contrary, combinations of substituents and/or variables are allowable only if such combinations are chemically allowed and result in a stable compound. A “stable” compound is a compound that can be prepared and isolated and whose structure and properties remain or can be caused to remain essentially unchanged for a period of time sufficient to allow use of the compound for the purposes described herein (e.g., therapeutic administration to a subject).

In some embodiments, the “optionally substituted” alkyl, alkylene, alkenyl, alkynyl, carbocyclic, carbocyclylene, cycloalkyl, cycloalkylene, alkoxy, cycloalkoxy, heterocyclyl, or heterocyclylene herein can each be independently unsubstituted or substituted with 1, 2, 3, or 4 substituents independently selected from F, Cl, —OH, protected hydroxyl, oxo (as applicable), NH₂, protected amino, NH(C₁₋₄ alkyl) or a protected derivative thereof, N(C₁₋₄ alkyl((C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2, or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy. In some embodiments, the “optionally substituted” aryl, arylene, heteroaryl or heteroarylene group herein can each be independently unsubstituted or substituted with 1, 2, 3, or 4 substituents independently selected from F, Cl, —OH, —CN, NH₂, protected amino, NH(C₁₋₄ alkyl) or a protected derivative thereof, N(C₁₋₄ alkyl((C₁₋₄ alkyl), —S(═O)(C₁₋₄ alkyl), —SO₂ (C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2 or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy, phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl, C₁₋₄ alkoxy and fluoro-substituted C₁₋₄ alkoxy.

“Halo” or “halogen” refers to fluorine (fluoro, —F), chlorine (chloro, —Cl), bromine (bromo, —Br), or iodine (iodo, —I).

The term “leaving group” is given its ordinary meaning in the art of synthetic organic chemistry and refers to an atom or a group capable of being displaced by a nucleophile. See, for example, Smith, March Advanced Organic Chemistry 6th ed. (501-502). Examples of suitable leaving groups include, but are not limited to, halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates.

The term “pharmaceutically acceptable salt”, “pharmaceutically acceptable anion” or “pharmaceutically acceptable cation” refers to those salts, anions or cations, which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts, anions, or cations are well known in the art.

The term “pharmaceutically acceptable ester” refers to those esters which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable esters are well known in the art, for example, a C₁₋₄ alkyl ester, such as ethyl ester.

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

The term “subject” (alternatively referred to herein as “patient”) as used herein, refers to an animal, preferably a mammal, most preferably a human, who has been the object of treatment, observation or experiment.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of a compound described herein to a subject in need of such treatment.

As used herein, the singular form “a”, “an”, and “the”, includes plural references unless it is expressly stated or is unambiguously clear from the context that such is not intended.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C(alone).

Headings and subheadings are used for convenience and/or formal compliance only, do not limit the subject technology, and are not referred to in connection with the interpretation of the description of the subject technology. Features described under one heading or one subheading of the subject disclosure may be combined, in various embodiments, with features described under other headings or subheadings. Further it is not necessarily the case that all features under a single heading or a single subheading are used together in embodiments.

### EXAMPLES

The various starting materials, intermediates, and compounds of the preferred embodiments can be isolated and purified where appropriate using conventional techniques such as precipitation, filtration, crystallization, evaporation, distillation, and chromatography. Characterization of these compounds can be performed using conventional methods such as by melting point, mass spectrum, nuclear magnetic resonance, and various other spectroscopic analyses. Exemplary embodiments of steps for performing the synthesis of products described herein are described in greater detail infra. The examples are illustrative only and do not limit the claimed invention in any way. Further, in the structures shown in the Examples section herein, a salt form and/or a counterion may be shown (the stoichiometry may or may not be shown, and the charge of the counterion may or may not be shown) to be associated with a particular structure. However, it should be understood that the Examples and/or Intermediates herein are not limited to any of the particular salt forms and/or counterions as drawn, for example, the Examples and/or Intermediates herein may exist in the form of an internal salt and/or an external salt with a pharmaceutically acceptable counterion.

The abbreviations used in the Examples section should be understood as having their ordinary meanings in the art unless specifically indicated otherwise or obviously contrary from context. The following shows a list of some of the abbreviations used in the Examples section and their ordinary meanings in the art:
AIBN azobisisobutyronitrile
ACN acetonitrile
Bn benzyl
DBU 1,8-Diazabicyclo [5.4.0]undec-7-ene
DCM dichloromethane
DEAD Diethyl azodicarboxylate
DHP 3,4-dihydropyran
DIBAL-H Diisobutylaluminium hydride
DMF dimethylformamide
DMP Dess-Martin periodinane
DMSO Dimethyl sulfoxide
DPPA Diphenylphosphoryl azide
Dppf 1,1′-Bis(diphenylphosphino) ferrocene
EA or EtOAc ethyl acetate
EDCI N-(3-Dimethylaminopropyl)-N′-ethylcarbodiimide
HMDS Hexamethyldisilazane
IPA isopropyl alcohol
LAH Lithium Aliminium hydride
LDA Lithium diisopropylamide
MTBE Methyl tertiary-butyl ether
NMP N-methylpyrrolidinone
NBS N-Bromosuccinimide
NIS N-Iodosuccinimide
O/N overnight
PCC pyridinium chlorochromate
PE petroleum ether
PPTS Pyridinium p-toluenesulfonate
Rt retention time (e.g., when describing HPLC peaks)
RT room temperature (describing reaction conditions)
TBAF tetra-n-butylammonium fluoride
TBS tert-butyldimethylsilyl (or TBDMS)
TBDPS tert-butyldiphenylsilyl
TEA triethyl amine
TFA trifluoroacetic acid
THF tetrahydrofuran
THP tetrahydropyran
TMS Trimethylsilyl
TPP triphenyl phosphine
TLC thin-layer chromatography
TsOH p-Toluenesulfonic acid (or PTSA)
Z benzyloxycarbonyl(benzyl chloroformate (Z—C1))

### Synthesis of Intermediates

### Synthesis of (2S,3R)-3-cyclopropyl-2-methyl-3-((R)-2-(piperidin-4-yl) chroman-7-yl)-propanoic acid, Intermediate 1

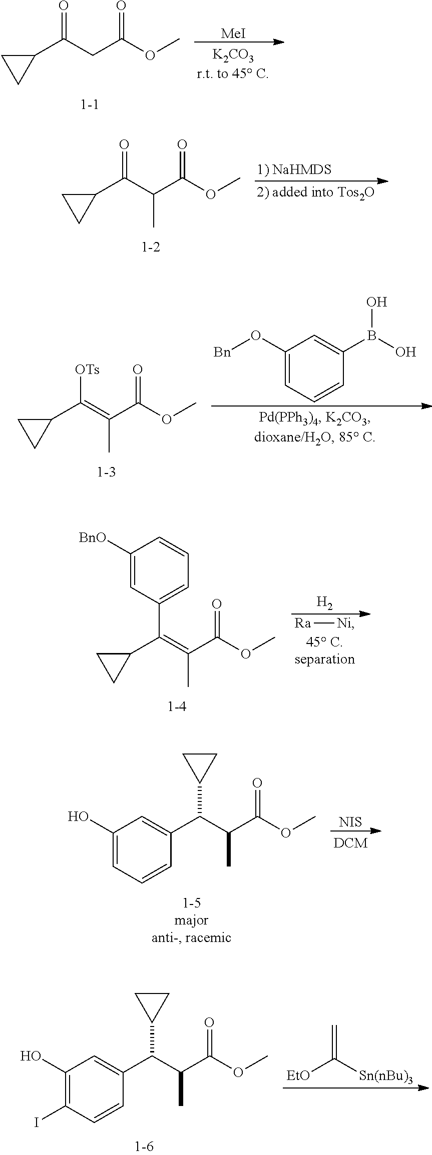

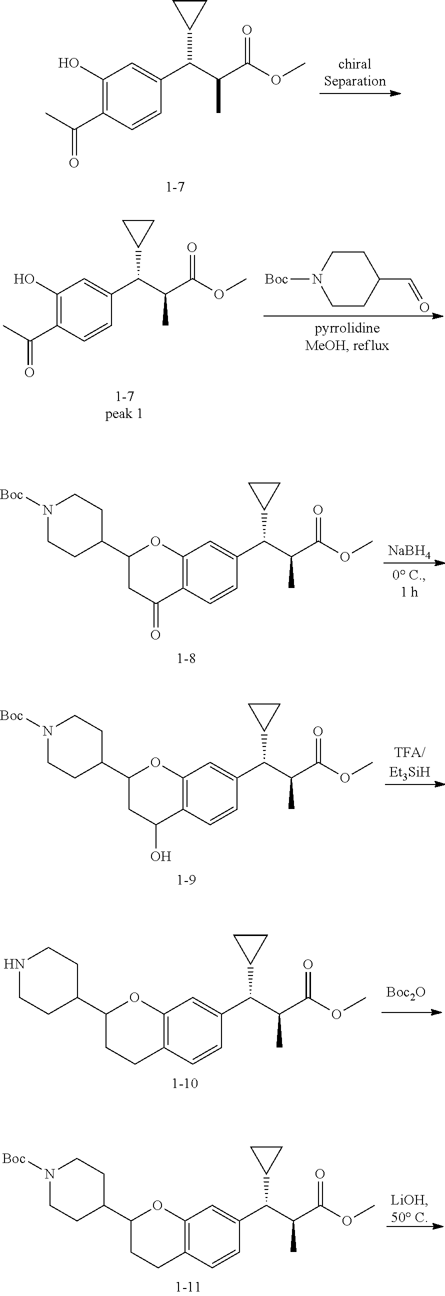

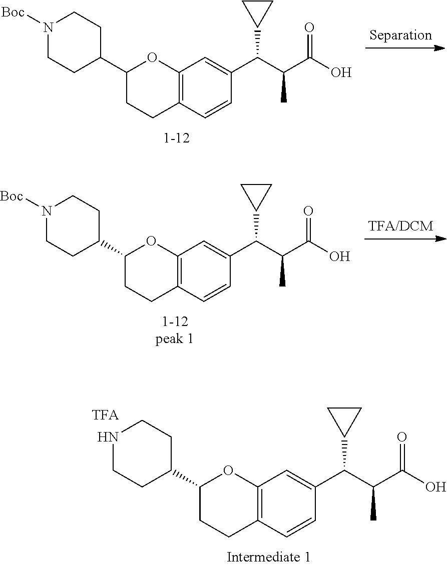 [SMILES: C[C@H](C(=O)O)[C@H](c1ccc2c(c1)OC(C1CCN(C(=O)OC(C)(C)C)CC1)CC2)C1CC1 ; C[C@H](C(=O)O)[C@H](c1ccc2c(c1)O[C@@H](C1CCN(C(=O)OC(C)(C)C)CC1)CC2)C1CC1 ; C[C@H](C(=O)O)[C@H](c1ccc2c(c1)O[C@@H](C1CCNCC1)CC2)C1CC1]

Step 1. To a mixture of K₂CO₃ (35 g, 0.253 mol) in THF (300 mL) at room temperature was added a solution of 1-1 (18 g, 0.127 mol) in THF (30 mL) dropwise in 15 min. The resulting mixture was stirred for 30 min at room temperature, followed by dropwise addition of a solution of CH₃I (8.8 mL, 0.139 mol) in THF (20 mL) in 15 min. The resulting mixture was stirred at 40° C. for 48 hrs. The reaction mixture was filtered, and the cake was washed with EA (200 mL×2). The organic phase was combined and concentrated. The residue was dissolved with EA (250 mL) and washed with brine (100 mL×2), dried and concentrated to give crude methyl 3-cyclopropyl-2-methyl-3-oxopropanoate (1-2) as a pale-yellow oil. ¹H NMR (400 MHZ, CDCl₃): δ=3.75 (s, 3H), 3.68 (q, J=7.2 Hz, 1H), 2.08-2.03 (m, 1H), 1.42 (d, J=7.2 Hz, 3H), 1.12-1.05 (m, 2H), 0.98-0.92 (m, 2H).

Step 2. To a solution of crude 1-2 (10 g, 0.064 mol) in THF (100 mL) at room temperature (20° C.) was added NaHMDS (2 M, 40 mL) dropwise in 20 min. The resulting mixture, after stirring for 30 min at room temperature, was added dropwise to a solution of Tos₂O (23 g, 0.07 mol) in THF (200 mL) at room temperature in 20 min. The resulting mixture was stirred for additional 16 h at 30° C. The reaction mixture was cooled with ice-water and quenched with aq. NH₄Cl (200 mL). The water phase was extracted with EA (100 mL×2). The combined organic phase was dried over Na₂SO₄ and concentrated to give a yellow residue, which was treated with IPA (40 mL) and cooled to 4° C. (in a refrigerator). White solid was collected as methyl (Z)-3-cyclopropyl-2-methyl-3-(tosyloxy) acrylate (1-3) by filtration. MS Calcd.: 310.1; MS Found: 311.1 [M+H]⁺.

Step 3. A flask charged with 1-3 (5 g, 0.0161 mol), (3-(benzyloxy)phenyl) boronic acid (4.76 g, 0.0209 mol), Pd(PPh₃)₄ (920 mg, 0.0008 mol) and K₂CO₃ (4.48 g, 0.0322 mol) in dioxane (85 mL) and water (12 mL) was degassed and filled with N2. The reaction mixture was heated at 85° C. for 16 hrs. Solvent was removed and the residue was purified by silica gel column chromatography (PE/EA=100:0 to 95:5) to give methyl (Z)-3-(3-(benzyloxy)phenyl)-3-cyclopropyl-2-methylacrylate (1-4) as a white solid. MS Calcd.: 322.2; MS Found: 323.3 [M+H]⁺. ¹H NMR (400 MHZ, CDCl₃): δ=7.43-7.31 (m, 5H), 7.16 (t, J=8.0 Hz, 1H), 6.85-6.83 (m, 1H), 6.59-6.55 (m, 2H), 5.03 (s, 2H), 3.34 (s, 3H), 2.13 (s, 3H), 1.84-1.80 (m, 1H), 0.73-0.69 (m, 2H), 0.32-0.28 (m, 2H).

Step 4. A mixture of 1-4 (1 g, 3.105 mmol) and Raney-Ni (˜500 mg, 50% w.t.) in MeOH (50 mL) was hydrogenation under H₂ (using a balloon) at 45° C. for 20 hrs. The mixture was filtered and the residue was purified by silica gel column chromatography (5% EA in PE) to give methyl(2S,3R)-3-cyclopropyl-3-(3-hydroxyphenyl)-2-methylpropanoate (1-5) as a white gum. MS Calcd.: 234.1; MS Found: 235.0 [M+H]⁺. ¹H NMR (400 MHZ, CDCl₃): δ=7.16 (t, J=8.0 Hz, 1H), 6.73-6.63 (m, 3H), 4.82 (brs, 1H), 3.72 (s, 3H), 2.81-2.77 (m, 1H), 1.88-1.57 (m, 1H), 1.05-1.00 (m, 1H), 0.94 (d, J=7.2 Hz, 3H), 0.57-0.51 (m, 2H), 0.31-0.18 (m, 1H), 0.001−−0.003 (m, 1H).

Step 5. To a mixture of 1-5 (1.45 g, 6.2 mmol) in DCM (100 mL) at room temperature was added NIS (1.68 g, 7.45 mmol) in portions. After the addition, the resulting mixture was stirred for 14 hr at room temperature. Solvent was removed and the residue was purified by silica gel column chromatography (PE/EA=100:0 to 90:10) to give methyl(2S,3R)-3-cyclopropyl-3-(3-hydroxy-4-iodophenyl)-2-methylpropanoate (1-6) as a pale-yellow solid. MS Calcd.: 360.0; MS Found: 360.8 [M+H]⁺. ¹H NMR (400 MHZ, CDCl₃): δ=7.56 (d, J=8.0 Hz, 1H), 6.82 (s, 1H), 6.50-6.48 (m, 1H), 5.35 (brs, 1H), 3.72 (s, 3H), 2.79-2.75 (m, 1H), 1.87 (t, J=10.0 Hz, 1H), 1.02-0.99 (m, 1H), 0.94 (d, J=6.8 Hz, 3H), 0.57-0.54 (m, 1H), 0.32-0.22 (m, 2H), −0.001−−0.005 (m, 1H).

Step 6. A flask charged with 1-6 (1.55 g, 4.305 mmol), tributyl(1-ethoxyvinyl) stannane (2.02 g, 5.597 mmol) and Pd(PPh₃)₄ (301 mg, 0.43 mmol) in toluene (100 mL) was degassed and filled with N2. The reaction mixture was heated at 100° C. for 16 hrs. Solvent was removed and the residue was purified by silica gel column chromatography (PE/EA=100:0 to 90:10) to give methyl(2S,3R)-3-(4-acetyl-3-hydroxyphenyl)-3-cyclopropyl-2-methylpropanoate (1-7) as a brown gum. MS Calcd.: 276.1; MS Found: 277.1 [M+H]⁺. ¹H NMR (400 MHZ, CDCl₃): δ=12.29 (s, 1H), 7.68 (d, J=8.4 Hz, 1H), 6.79 (s, 1H), 6.72-6.70 (m, 1H), 3.73 (s, 3H), 2.85-2.80 (m, 1H), 2.60 (s, 3H), 1.94 (t, J=9.6 Hz, 1H), 1.05-1.02 (m, 1H), 0.96 (d, J=6.8 Hz, 3H), 0.60-0.58 (m, 1H), 0.35-0.24 (m, 2H), 0.01−−0.006 (m, 1H).

Step 7. Product 1-7-1 was obtained by chiral separation (Column: ChiralPak IA from Daicel, mobile phase: ACN: IPA 90%: 10%) as peak 1, Rt=3.7 min. The other isomer 1-7-2 eluted as peak 2, Rt=7.6 min.

Step 8. To a stirred mixture of 1-7-1 (250 mg, 0.9 mmol) in MeOH (15 mL) was added tert-butyl 4-formylpiperidine-1-carboxylate (230 mg, 1.08 mmol) and pyrrolidine (96 mg, 1.35 mmol). The resulting mixture was then heated at 65° C. for 12 hrs. Solvent was removed and the residue was purified by silica gel column chromatography (PE/EA=100:0 to 80:20) to give tert-butyl 4-(7-((1R,2S)-1-cyclopropyl-3-methoxy-2-methyl-3-oxopropyl)-4-oxochroman-2-yl) piperidine-1-carboxylate (1-8) as a white gum. MS Calcd.: 471.2; MS Found: 494.2 [M+Na]⁺.

Step 9. To a stirred mixture of 1-8 (400 mg, 0.85 mmol) in MeOH (10 mL) at 0° C. was added NaBH₄ (48 mg, 1.27 mmol) in small portions. The resulting mixture was stirred for 1 hr, allowing the temperature to slowly warm to room temperature. Solvent was removed and the residue was treated with EA (50 mL), washed with brine, dried and concentrated to give tert-butyl 4-(7-((1R,2S)-1-cyclopropyl-3-methoxy-2-methyl-3-oxopropyl)-4-hydroxychroman-2-yl) piperidine-1-carboxylate (1-9) as a yellow gum. MS Calcd.: 473.3; MS Found: 496.3 [M+Na]⁺.

Step 10. To a stirred mixture of crude 1-9 (400 mg, 0.85 mmol) in DCM (8 mL) was added TFA (2 mL) and stirred for 20 min at room temperature. Et₃SiH (0.6 mL, 4.25 mmol) was added dropwise. The resulting mixture was stirred for additional 12 hrs at room temperature. Solvent was removed and the residue was basified with aq. NaHCO₃until pH reached 8 to 9, extracted with EA (20 mL×3), dried and concentrated to give methyl(2S,3R)-3-cyclopropyl-2-methyl-3-(2-(piperidin-4-yl) chroman-7-yl) propanoate (1-10) as a yellow gum. MS Calcd.: 357.2; MS Found: 358.2 [M+H]⁺.

Step 11. To a stirred mixture of crude 1-10 (400 mg, 0.85 mmol) in DCM (20 mL) and MeOH (5 mL) was added TEA (260 mg, 2.55 mmol)) and Boc₂O (280 mg, 1.27 mmol) at room temperature. The resulting mixture was stirred at room temperature for 6 hrs. Solvent was removed and the residue was purified by silica gel column chromatography (PE/EA=100:0 to 80:20) to give tert-butyl 4-(7-((1R,2S)-1-cyclopropyl-3-methoxy-2-methyl-3-oxopropyl) chroman-2-yl) piperidine-1-carboxylate (1-11) as a colorless gum. MS Calcd.: 457.3; MS Found: 480.2 [M+Na].

Step 12. A mixture of 1-11 (500 mg, 1.09 mmol) and LiOH·H₂O (470 mg, 10.9 mmol) in MeOH (15 mL)/THF (15 mL)/water (15 mL) was heated at 50° C. for 48 hrs. Volatiles were removed and the aqueous layer was acidified with 1M HCl until pH reached 3 to 4, extracted with EA (30 mL×4), dried and concentrated. The residue was purified by chromatography to give (2S,3R)-3-(2-(1-(tert-butoxycarbonyl) piperidin-4-yl) chroman-7-yl)-3-cyclopropyl-2-methyl propanoic acid (1-12) as a white solid. MS Calcd.: 443.3; MS Found: 466.2 [M+Na]⁺.

Step 13. Product 1-12-1 was obtained by chiral separation (Method Info: Column: ChiralpakAD-H, Mobile phase: Hex: EtOH: TFA=90:10: 0.2) as peak 1, Rt=7.9 min. The other isomer 1-12-2 was obtained as peak 2, Rt=9.4 min.

Step 14. To a mixture of 1-12-1 (150 mg, 0.337 mmol) in DCM (6 mL) at room temperature was added TFA (1.5 mL) dropwise. The resulting mixture was stirred for 2 hrs at room temperature. Volatiles were removed in vacuum to give (2S,3R)-3-cyclopropyl-2-methyl-3-((R)-2-(piperidin-4-yl) chroman-7-yl) propanoic acid as a TFA salt, Intermediate 1. MS Calcd.: 343.2; MS Found: 344.1 [M+H]⁺.]+. ¹H NMR (400 MHZ, DMSO-d6): δ 12.17 (br, 1H), 8.76 (br, 1H), 8.40 (br, 1H), 6.97 (d, J=8.0 Hz, 1H), 6.66 (d, J=8.0 Hz, 1H), 6.53 (s, 1H), 3.83-3.79 (m, 1H), 3.38-3.33 (m, 2H), 2.92-2.89 (m, 2H), 2.78-2.62 (m, 3H), 2.06-1.95 (m, 2H), 1.88-1.83 (m, 3H), 1.66-1.24 (m, 3H), 1.09-1.03 (m, 1H), 0.82 (d, J=6.8 Hz, 3H), 0.55-0.45 (m, 1H), 0.30-0.20 (m, 2H), −0.05−−0.15 (m 1H).

### Synthesis of Intermediate 2

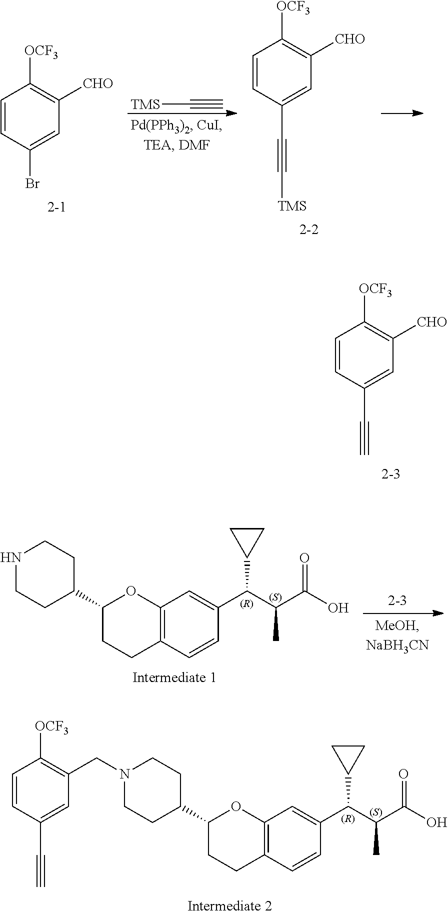

Step 1. To a solution of 5-bromo-2-(trifluoromethoxy)benzaldehyde (2-1)(1.0 g, 3.717 mmol) in DMF (20 mL) was added ethynyltrimethylsilane (730.0 mg, 7.434 mmol), CuI (141.0 mg, 0.744 mmol), TEA (1.88 g, 18.585 mmol) and Pd(PPh₃)Cl₂ (261.0 mg, 0.372 mmol) under nitrogen atmosphere. The mixture was stirred at room temperature for 16 hours. The reaction mixture was quenched with water and extracted with EA (50 mL×3). The combined organic layers were washed with water (50 mL×2) and brine (50 mL), dried over Na₂SO₄, and concentrated in vacuum (35° C.). The residue was purified by flash chromatography (PE) to give 2-(trifluoro-methoxy)-5-((trimethylsilyl) ethynyl)benzaldehyde (2-2) as a yellow oil. ¹H NMR (400 MHZ, CDCl₃): δ=10.33 (s, 1H), 8.03 (d, J=2 Hz, 1H), 7.70 (dd, J=2, 9 Hz, 1H), 7.29 (d, J=9 Hz, 1H), 0.26 (s, 9H).

Step 2. To a solution of 2-2 (1.0 g, 3.493 mmol) in MeOH/H₂O (15/5 mL) was added KOH (587.0 mg, 10.479 mmol) at room temperature. The mixture was stirred at room temperature overnight. The reaction mixture was quenched with water and extracted with EA (50 mL×3). The combined organic layers were washed with water (50 mL×2), brine (50 mL), dried over Na₂SO₄ and concentrated in vacuum (35° C.). The residue was purified by flash chromatography (PE) to give 5-ethynyl-2-(trifluoromethoxy)benzaldehyde (2-3) as a yellow oil. ¹H NMR (400 MHZ, CDCl₃): δ=10.34 (s, 1H), 8.06 (d, J=2 Hz, 1H), 7.74 (dd, J=2, 9 Hz, 1H), 7.29 (d, J=9 Hz, 1H), 3.17 (s, 1H).

Step 3. To a solution of Intermediate 1 (20.0 mg, 0.044 mmol) in MeOH (5 mL) was added compound 2-3 (28.0 mg, 0.131 mmol). The mixture was stirred at 35° C. for 6 hrs, followed by the addition of NaBH₃CN(8.3 mg, 0.131 mmol). The resulting mixture was stirred overnight. After concentration in vacuum the residue was purified by prep-HPLC (0.1% NH₄OAc as additive) to give (2S,3R)-3-cyclopropyl-3-((R)-2-(1-(5-ethynyl-2-(trifluoro-methoxy)benzyl) piperidin-4-yl)-chroman-7-yl)-2-methylpropanoic acid, Intermediate 2, as a white solid. ¹H NMR (400 MHZ, CDCl₃): δ=7.71 (d, J=1.6 Hz, 1H), 7.41 (dd, J=8.8, 2.0 Hz, 1H), 7.19 (dd, J=8.8, 1.2 Hz, 1H), 6.92 (d, J=7.2 Hz, 1H), 6.63-6.59 (m, 2H), 3.81-3.78 (m, 1H), 3.71-3.59 (q, 2H), 3.12 (d, 1H), 3.07 (s, 1H), 2.99 (d, 1H), 2.84-2.69 (m, 3H), 2.15 (m, 2H), 1.9 (m, 3H), 1.85-1.57 (m, 5H), 1.1 (m, 1H), 0.98 (d, J=7.2 Hz, 3H), 0.60-0.58 (m, 1H), 0.38-0.35 (m, 1H), 0.27-0.23 (m, 1H), −0.03 ˜−0.05 (m, 1H). MS: m/z 542.3 (M+H⁺).

### Synthesis of Intermediate 3

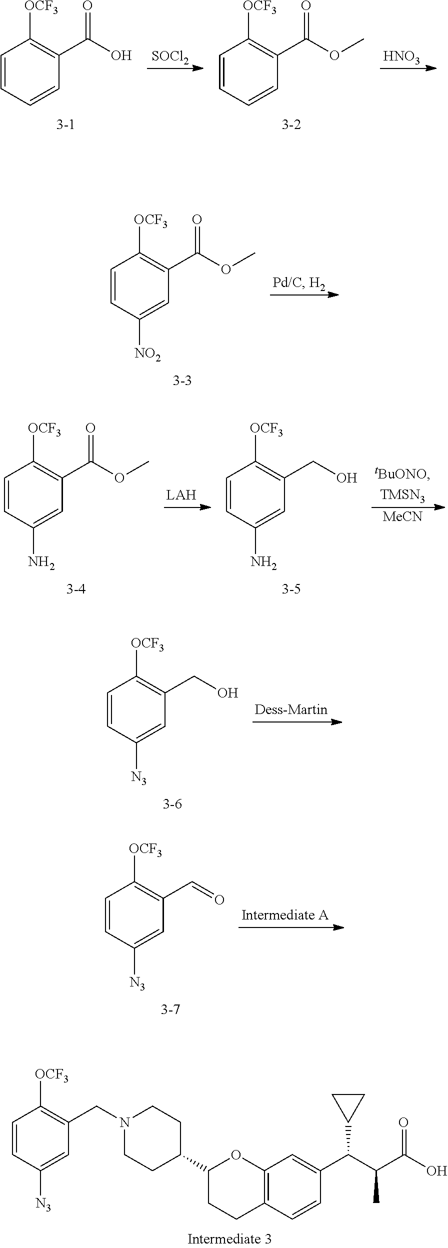

Step 1. To a solution of 2-(trifluoromethoxy)benzoic acid (3-1)(2.55 g, 12.4 mmol) in MeOH (120 mL) was added SOCl₂ (1.32 mL, 18.6 mmol) at 0° C. under nitrogen atmosphere. The mixture was heated at 65° C. overnight. The reaction mixture was evaporated to dryness (residual SOCl₂ was azeotropically removed under reduced pressure with toluene). The residue was diluted with EA (50 mL), washed with aq. NaHCO₃ (30 mL×2) and brine, dried over Na₂SO₄ and concentrated to give crude methyl 2-(trifluoromethoxy)benzoate (3-2) as a pale yellow liquid. ¹H NMR (400 MHZ, CDCl₃): δ=7.95 (dd, J=7.6, 1.6 Hz, 1H), 7.55 (dt, J=8.0, 1.6 Hz, 1H), 7.37 (dt, J=7.6, 0.8 Hz, 1H), 7.33 (d, J=8.0 Hz, 1H), 3.93 (s, 3H). MS: m/z 221.0 (M+H⁺).

Step 2. A solution of 3-2 (2.04 g, 9.3 mmol) in concentrated H₂SO₄ (32 mL) was stirred at 0° C. for 15 min, followed with addition of HNO₃ (4.2 mL)/H₂SO₄ (15.8 mL) dropwise at 0° C. The mixture was stirred for 2 hrs. The reaction mixture was poured into 100 mL of ice water and the aqueous phase was extracted with EA (50 mL×3). The combined organic layers were washed with aq. NaOH (1g dissolved in 50 mL of water) and brine (50 mL), dried over Na₂SO₄ and concentrated to give methyl 5-nitro-2-(trifluoromethoxy)benzoate (3-3) as an orange liquid. The crude was used for next step without further purification. ¹H NMR (400 MHZ, CDCl₃): δ=8.83 (d, J=3 Hz, 1H), 8.44 (dd, J=9, 3 Hz, 1H), 7.54 (d, J=9 Hz, 1H), 4.00 (s, 3H).

Step 3. A mixture of 3-3 (3.53 g, 9.25 mmol) and Pd/C (530 mg) in MeOH (100 mL) was stirred under hydrogen balloon atmosphere at room temperature for 16 hrs. The reaction mixture was filtered over Celite and concentrated. The residue was purified by silica gel column chromatography (PE:EA=3:1) to give methyl 5-amino-2-(trifluoromethoxy)benzoate (3-4) as a pale yellow liquid. ¹H NMR (400 MHZ, CDCl₃): δ=7.19 (d, J=3 Hz, 1H), 7.09 (dd, J=9, 3 Hz, 1H), 6.79 (d, J=9 Hz, 1H), 3.90 (s, 3H). MS: m/z 236.0 (M+H⁺).

Step 4. To a mixture of 3-4 (490 mg, 2.085 mmol) in dry THF (12 mL) was added LiAlH₄ (150 mg, 4.17 mmol) in small portions at 0° C. under N₂ atmosphere. After addition, the resulting mixture was stirred for 4 hrs at room temperature. The reaction mixture was quenched with water (0.15 mL), aq. NaOH (15%, 0.15 mL) and water (0.45 mL) successively. EA (50 mL) and Na₂SO₄ (˜5 g) was added into the mixture and stirred for 15 min. The mixture was then filtered and the filter cake was washed with EA (20 mL×2). The organic phase was combined and concentrated. The residue was purified by silica gel column chromatography (PE:EA=70:30) to give (5-amino-2-trifluoromethoxy-phenyl)-methanol (3-5) as a yellow oil. MS Calcd.: 207.0; MS Found: 207.9 [M+H]⁺. ¹H NMR (400 MHZ, DMSO-d6): δ=6.90 (dd, J=8.8, 1.2 Hz, 1H), 6.74 (d, J=2.8 Hz, 1H), 6.45 (dd, J=8.4, 2.8 Hz, 1H), 5.26 (brs, 2H), 5.17 (t, J=5.6 Hz, 1H), 4.41 (d, J=5.6 Hz, 2H).

Step 5. To a solution of 3-5 (300 mg, 1.45 mmol) in acetonitrile (20 mL) was added tert-butyl nitrite (300 mg, 2.9 mmol) and TMSN₃ (250 mg, 2.175 mmol) successively at 0° C. under nitrogen atmosphere. After addition, the resulting mixture was allowed to warm to room temperature and stirred for 3 hrs. Volatiles were evaporated and the residue was treated with EA (50 mL), washed with water (20 mL×2), dried and concentrated to give crude (5-azido-2-trifluoromethoxy-phenyl)-methanol (3-6) as yellow solid. ¹H NMR (400 MHz, CDCl₃): δ=7.26 (s, overlap, 1H), 7.22-7.20 (m, 1H), 6.96-6.93 (m, 1H), 4.77 (s, 2H).

Step 6. To a mixture of crude 3-6 (100 mg, 0.43 mmol) in DCM (8 mL) was added DMP (273 mg, 0.64 mmol) in portions. After addition, the resulting mixture was stirred for 6 hrs at room temperature. Solvent was removed and the residue was purified by flash chromatography (PE:EA=95:5) to give 5-azido-2-trifluoromethoxy-benzaldehyde (3-7) as a yellow oil. ¹H NMR (400 MHZ, CDCl₃): δ=10.33 (s, 1H), 7.62 (d, J=2.8 Hz, 1H), 7.37-7.35 (m, 1H), 7.27-7.25 (m, 1H).

Step 7. (2S,3R)-3-((S)-2-(1-(5-azido-2-(trifluoromethoxy)benzyl) piperidin-4-yl) chroman-7-yl)-3-cyclopropyl-2-methylpropanoic acid, Intermediate 3, was prepared from 3-7 and Intermediate 1 in the same way as Intermediate 2. MS Calcd.: 558.2; MS Found: 559.3 [M+H]⁺. ¹H NMR (400 MHZ, CDCl₃): δ=7.28 (d, 1H), 7.20 (d, 1H), 6.94-6.92 (m, 2H), 6.62-6.60 (m, 2H), 3.79-3.76 (m, 1H), 3.64-3.55 (q, 2H), 3.04-2.94 (m, 2H), 2.80-2.73 (m, 3H), 2.26-1.88 (m, 5H), 1.80-1.60 (m, 5H), 1.10-1.04 (m, 1H), 0.98 (d, J=6.4 Hz, 3H), 0.62-0.55 (m, 1H), 0.37-0.26 (m, 2H), −0.01−−0.03 (m, 1H).

### Synthesis of Intermediate 4

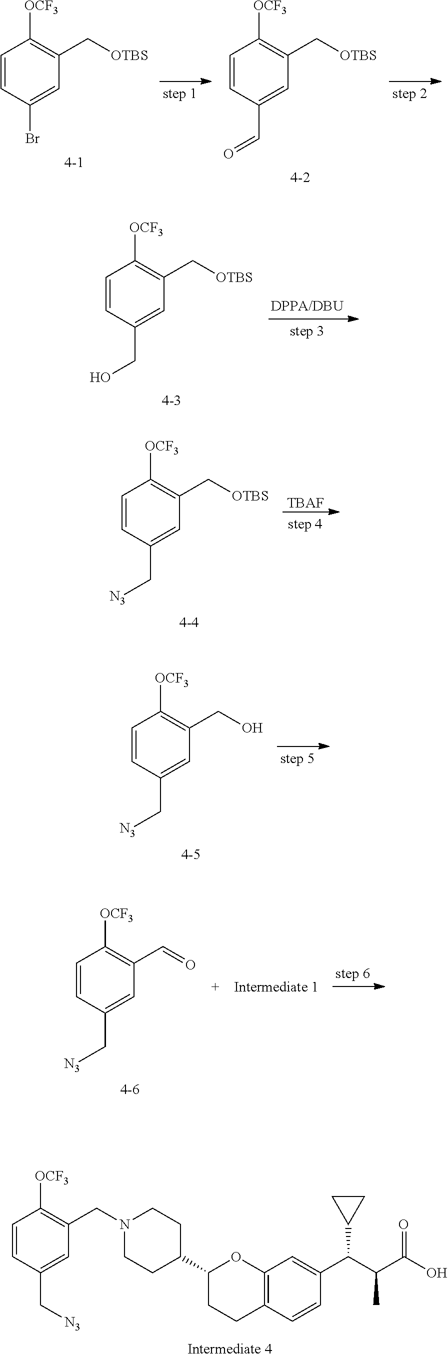

Step 1. To a solution of ((5-bromo-2-(trifluoromethoxy)benzyl)oxy)(tert-butyl)dimethyl-silane (12.5 g, 0.032 mol) in THF (100 mL) was added n-BuLi (2.5M, 16 mL, 0.039 mmol) at −78° C. for 1.5 hrs, followed by the addition of DMF (2.6 g, 0.036 mmol) at −78° C. The mixture was stirred at −78° C. for 2 hrs, and quenched with saturated aqueous NH₄Cl (100 mL), and extracted with EA (100 mL×2). The organic layer was washed with brine (100 mL), dried over Na₂SO₄ and filtered. The filtrate was concentrated, and the residue was purified by silica gel column chromatography (PE/EA=30/1, v/v) to afford 3-(((tert-butyldimethyl silyl)oxy)methyl)-4-(trifluoromethoxy)benzaldehyde (4-2) as a yellow oil. ¹H NMR (400 MHZ, DMSO-d6): δ=9.93 (s, 1H), 7.99-7.98 (m, 1H), 7.89-7.87 (m, 1H), 7.49-7.47 (m, 1H), 4.70 (s, 2H), 0.80 (s, 9H), 0.01 (s, 6H).

Step 2. To a solution of 4-2 (4 g, 11.9 mmol) in MeOH (50 mL) at 0° C. was added NaBH₄ (906 mg, 23.9 mmol), the resulting mixture was stirred for 2 hrs. The reaction was quenched with saturated aqueous NH₄Cl (50 mL) and extracted with EA (50 mL×2). The organic layer was dried over Na₂SO₄, filtered and concentrated and the residue was purified by silica gel column chromatography (PE/EA=5/1, v/v) to afford (3-(((tert-butyldimethylsilyl)oxy)methyl)-4-(trifluoromethoxy)phenyl) methanol (4-3) as a colorless oil. ¹H NMR (400 MHZ, DMSO-d6): δ=7.43 (d, J=1.6 Hz, 1H), 7.26-7.23 (m, 1H), 7.20-7.18 (m, 1H), 5.20 (s, 1H), 4.64 (s, 2H), 4.43 (s, 2H), 0.81 (s, 9H), 0.01 (s, 6H).

Step 3. To a solution of 4-3 (500 mg, 1.49 mmol) in DMF (10 mL) was added DPPA (491 mg, 1.79 mmol) and DBU (270 mg, 1.79 mmol) at RT. The mixture was stirred at 90° C. for overnight. The residue was poured into water (10 mL), extracted with EA (20 mL×2). The organic layer was washed with brine (100 mL×2), dried over Na₂SO₄ and filtered. The filtrate was concentrated, and the residue was purified by silica gel column chromatography (PE) to afford ((5-(azidomethyl)-2-(trifluoromethoxy)benzyl)oxy)(tert-butyl)dimethylsilane (4-4) as a colorless oil.

Step 4. To a solution of 4-4 (170 mg, 0.47 mmol) in THF (100 mL) was added TBAF (1.0M, 1 mL, 0.94 mmol) at room temperature. The mixture was stirred for 2 h. The mixture was concentrated in vacuo to afford (5-(azidomethyl)-2-(trifluoromethoxy)phenyl) methanol (4-5) as a yellow oil.

Step 5. To a solution of 4-5 (120 mg, 0.49 mmol) in DCM (6 mL) was added DMP (412 mg, 0.97 mmol) at ice-bath and the mixture was stirred at room temperature for 2 hrs. The reaction was concentrated in vacuo. The residue was purified by silica gel column chromatography (PE/EA=20/1, v/v) to afford 5-(azidomethyl)-2-(trifluoro methoxy)benzaldehyde (4-6) as a colorless oil. ¹H NMR (400 MHZ, CDCl₃): δ=10.38 (s, 1H), 7.92 (d, J=2.4 Hz, 1H), 7.65-7.62 (m, 1H), 7.41-7.38 (m, 1H), 4.45 (s, 2H).

Step 6. (2S,3R)-3-((R)-2-(1-(5-(azidomethyl)-2-(trifluoromethoxy)benzyl) piperidin-4-yl) chroman-7-yl)-3-cyclopropyl-2-methylpropanoic acid, Intermediate 4, was prepared from 4-6 and intermediate 1 in the same way as Intermediate 2. 1H NMR (400 MHZ, CD₃OD): δ=7.68 (s, 1H), 7.48 (d, J=9.2 Hz, 1H), 7.41 (d, J=8.8 Hz, 1H), 7.00 (d, J=8.0 Hz, 1H), 6.67 (d, J=7.6 Hz, 1H), 6.60 (s, 1H), 4.49 (s, 2H), 3.80 (m, 3H), 3.14 (m, 2H), 2.85-2.72 (m, 3H), 2.33-2.22 (m, 2H), 2.08-2.05 (m, 3H), 1.83-1.60 (m, 5H), 1.11-1.09 (m, 1H), 0.96-0.93 (d, 3H), 0.62-0.60 (m, 1H), 0.39-0.30 (m, 2H), 0.01 ˜−0.02 (m, 1H).

### Synthesis of(S)-3-cyclopropyl-3-(3-hydroxyphenyl) propanoic acid, Intermediate 5

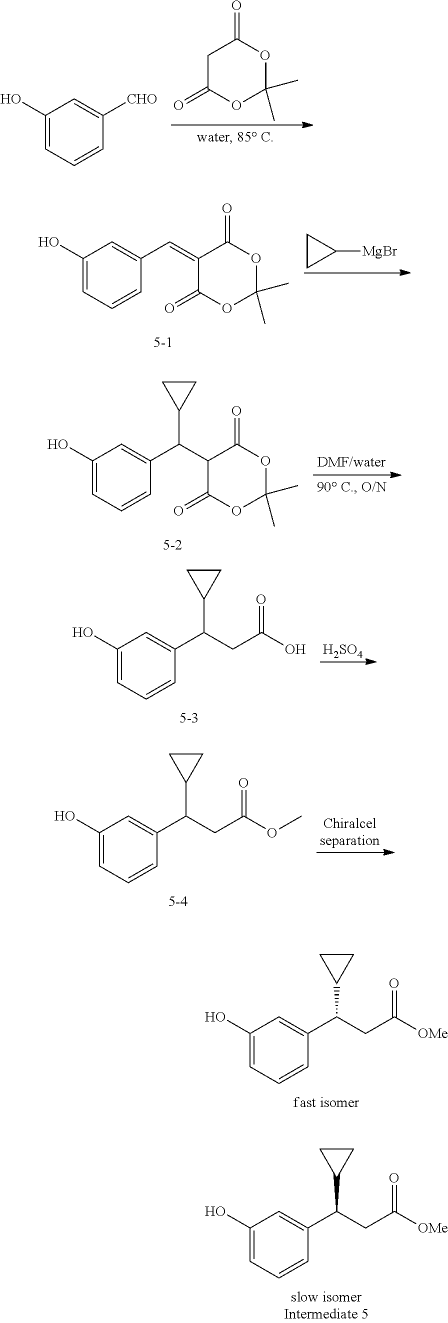

Step 1. A mixture of 3-hydroxybenzaldehyde (15.0 g, 122.95 mmol) in water (120 mL) was heated at 85° C. for 10 min until the mixture became clear. Then 2,2-dimethyl-1,3-dioxane-4,6-dione (17.7 g, 122.95 mmol) was added in 3 portions. After addition the resulting mixture was stirred at 85° C. for 1.5 h. Heating was stopped and the reaction mixture was cooled naturally with stirring. 5-(3-hydroxybenzylidene)-2,2-dimethyl-1,3-dioxane-4,6-dione, 5-1, (26.3 g, 86.3%) was collected by filtration as yellow solid. ¹H NMR (400 MHZ, CDCl₃) δ 9.79 (s, 1H), 8.37 (s, 1H); 7.78 (t, J=1.8 Hz, 1H); 7.48 (d, J=8.0 Hz, 1H); 7.38 (t, J=8.0 Hz, 1H); 7.09-7.07 (m, 1H); 5.73 (s, 1H); 1.80 (s, 6H).

Step 2. To a solution of 5-1 (4.0 g, 16.13 mmol) in THF (60 mL) under Nitrogen was added dropwise cyclopropylmagnesium bromide (1.0 M, 80 mL, 80.65 mmol) at 0° C. The reaction mixture was warmed to RT stirred for 1.5 h. The reaction mixture was cooled to 0° C. and quenched by 1 N HCl until pH reached 5 to 6. The mixture was separated and the water phase was extracted with EA (50 ml×3). The organic layer was combined, dried by anhydrous Sodium sulfate and evaporated. The residue was purified by silica-gel column chromatography (PE/EA=5/1) to give 5-(cyclopropyl(3-hydroxyphenyl)methyl)-2,2-dimethyl-1,3-dioxane-4,6-dione, 5-2, (2.0 g, 42.7%) as yellow oil. ¹H NMR (400 MHZ, CDCl₃) δ: 9.30 (s, 1H), 7.18 (t, J=7.8 Hz, 1H), 6.75 (s, 1H), 6.69 (d, 1H), 6.70 (m, 1H), 4.56 (s, 1H), 2.69 (m, 1H), 1.75 (s, 3H), 1.74 (m, 1H), 1.44 (s, 3H), 0.60 (m, 2H), 0.37 (m, 1H), 0.13 (m, 1H).

Step 3. A mixture of 5-2 (2.0 g, 6.90 mmol) in DMF/water (30 mL/3 mL) was heated at 90° C. for overnight. The reaction mixture was diluted with water and extracted with ethyl acetate. The organic layer was washed with brine, dried by anhydrous sodium sulfate and evaporated. The residue was purified by silica-gel column chromatography (Petroleum ether/Ethyl acetate=2/1) to give 3-cyclopropyl-3-(3-hydroxyphenyl) propanoic acid, 5-3, as a yellow oil. MS (ESI) m/z=207.0 [M+H]⁺

Step 4. To a stirred solution of 5-3 (1.0 g, 4.85 mmol) in MeOH (30 mL) was added H₂SO₄ (conc., 0.5 mL, 9.2 mmol) dropwise at 0° C. The resulting mixture was stirred at 70° C. for 12 h. After the completion of reaction, MeOH was removed and the mixture was diluted with water (50 mL), extracted with EA (30 mL×3) and concentrated. The residue was purified by silica-gel column chromatography (Petroleum ether/Ethyl acetate=10/1) to give methyl 3-cyclopropyl-3-(3-hydroxyphenyl) propanoate, 5-4, (680 mg, 63.7%) as white solid. MS (ESI) m/z=221.0 [M+H]⁺. ¹H NMR (400 MHZ, DMSO) δ 9.24 (s, 1H), 7.06 (t, J=7.8 Hz, 1H), 6.81 (d, J=7.6 Hz, 1H), 6.72-6.71 (m, 1H), 6.67-6.64 (d, 1H), 6.63 (s, 1H), 3.51 (s, 3H), 2.73-2.62 (m, 2H), 2.21-2.15 (m, 1H), 0.99-0.95 (m, 1H), 0.51-0.45 (m, 1H), 0.40-0.36 (m, 1H), 0.19-0.14 (m, 1H), 0.12-0.08 (m, 1H).

Step 5. Intermediate 5 was obtained by chiral separation (Column: ChiralpakOD-H, Mobile phase: Hex: EtOH=95:5, peak 1, Rt=9.5), The other enantiomer eluted out as peak 2 at 11.2 min.

### Synthesis of Intermediate 6

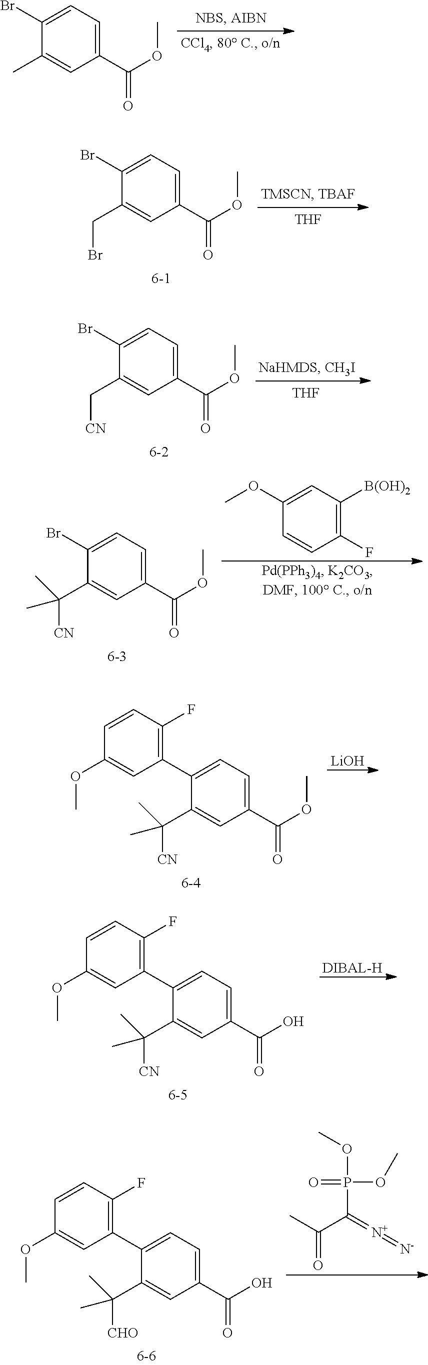

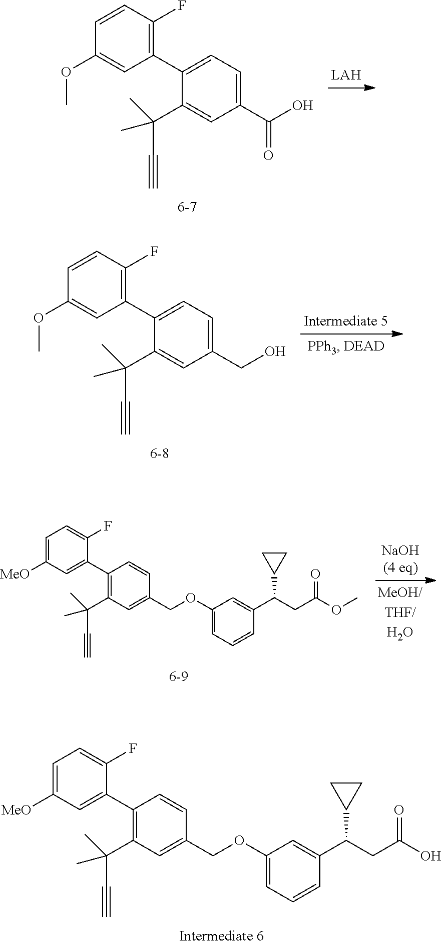 [SMILES: C#CC(C)(C)c1cc(C(=O)O)ccc1-c1cc(OC)ccc1F ; C#CC(C)(C)c1cc(CO)ccc1-c1cc(OC)ccc1F ; C#CC(C)(C)c1cc(COc2cccc([C@@H](CC(=O)OC)C3CC3)c2)ccc1-c1cc(OC)ccc1F ; C#CC(C)(C)c1cc(COc2cccc([C@@H](CC(=O)O)C3CC3)c2)ccc1-c1cc(OC)ccc1F]

Step 1. To a solution of methyl 4-bromo-3-methyl benzoate (1.0 g, 4.49 mmol) in CCl₄ (30 mL) at room temperature was added NBS (838 mg, 1.05 mmol), AIBN(74 mg, 0.449 mmol). The reaction mixture was stirred at 80° C. overnight under N₂. The reaction mixture was diluted with DCM and water. The aqueous phase was extracted with DCM (15 mL×4). The combined organic layers were dried over MgSO₄ and concentrated. The crude product was chromatographed on silica gel (Petroleum ether/EtOAc=10:1) to give compound, 6-1 as a white solid. ¹H NMR (400 MHZ, CDCl₃) δ 8.12 (d, J=2 Hz, 1H), 7.82 (dd, J=8.2, 2.2 Hz, 1H), 7.67 (d, J=8.4 Hz, 1H), 4.62 (s, 2H), 3.93 (s, 3H).

Step 2. To a solution of TMSCN(3861 mg, 39 mmol) in dry THF (30 mL) at 0° C. was added TBAF (35.8 mL, 35.8 mmol) under N₂. After 1 hour, the reaction mixture was added 6-1 (10 g, 32.5 mmol) in ACN(200 mL). The reaction mixture was stirred at 80° C. under N₂ for 1 hour. The reaction mixture was concentrated under reduced pressure to remove THF and ACN. The crude product was chromatographed on silica gel (Petroleum ether/EtOAc=20:1 to 10:1 to 5:1 to 3:1) to give compound 6-2 (6.3 g, 70%) as a white solid. ¹H NMR (400 MHz, CDCl₃) δ 8.17 (s, 1H), 7.89 (d, J=11.2 Hz, 1H), 7.71 (d, J=10.8 Hz, 1H), 3.94 (s, 3H), 3.89 (s, 2H).

Step 3. To a solution of 6-2 (4.65 g, 18.3 mmol) in THF (120 mL) at 0° C. under N₂ was added NaHMDS (2 M, 36.3 mL) dropwise during 15 min. Stirred for 30 min at 0° C. Then Mel (10.4 g, 73.2 mmol) was added dropwise at 0° C. The resulting mixture was stirred for 1 h at 0° C. and for 14 hrs at r. t. The reaction mixture was quenched by aq. NH₄Cl at 0° C. and separated. The water phase was extracted with EA (100 mL×2). The organic phase was combined and washed with brine, dried over Na₂SO₄ and concentrated to give crude 4-bromo-3-(cyano-dimethyl-methyl)-benzoic acid 6F-3 (4.6 g, yield: 89%) as yellow solid. ¹H NMR (400 MHZ, CDCl₃) δ 8.11 (d, J=1.6 Hz, 1H), 8.85-8.83 (dd, 1H), 7.76 (d, J=8.4 Hz, 1H), 3.94 (s, 3H), 1.93 (s, 6H).

Step 4. A mixture of 6-3 (2.3 g, 8.156 mmol), 2-fluoro-5-methoxyphenylboronic acid (2.08 g, 12.234 mmol), (PPh₃)₄ (942 mg, 0.081 mmol) and K₂CO₃ (3.4 g, 24.47 mmol) in DMF (100 mL) was degassed and filled with N₂ 3 times and heated at 105° C. for 16 hrs. DMF was removed and the residue was diluted with water (100 mL), extracted with EA (60 mL×3), dried and concentrated. The residue was purified by flash chromatography (70EA in PE) to give 2-(cyano-dimethyl-methyl)-2′-fluoro-5′-methoxy-biphenyl-4-carboxylic acid, 6-4, (4.1 g, yield: 76%) as yellow solid. ¹H NMR (400 MHZ, CDCl₃) δ 8.27 (d, J=1.2 Hz, 1H), 8.01 (dd, J=8.0 Hz, 1.6 Hz, 1H), 7.27-7.25 (dd, 1H), 7.08-7.03 (t, 1H), 6.95-6.91 (m, 1H), 6.86-6.83 (m, 1H), 3.96 (s, 3H), 3.80 (s, 3H), 1.76 (s, 3H), 1.65 (s, 3H).

Step 5. A mixture of 6-4 (3.1 g, 9.48 mmol) and LiOH·H₂O (800 mg, 18.96 mmol) in MeOH (10 mL), THF (30 mL) and water (10 mL) was stirred for 14 hrs at r. t. Solvent was removed and the residue was acidified with 1N HCl until the pH reached 2 to 3, extracted with EA (50 mL×3), dried and concentrated to give the crude 2-(cyano-dimethyl-methyl)-2′-fluoro-5′-methoxy-biphenyl-4-carboxylic acid, 6-5, as a brown solid, which was directly used in the next step.

Step 6. To a mixture of crude 6-5 (2.85 g, 9.1 mmol) in toluene (140 mL) at −78° C. was added DIBAL-H (1.0 M in hexane, 21 mL) dropwise during 15 min. After addition the resulting mixture was stirred for 1 h at −78° C. and stirred for additional 16 hrs at r. t. The reaction mixture was quenched by aq. NH₄Cl at 0° C. and further acidified with 1N HCl (about 50 mL), extracted with EA (50 mL×4), dried and concentrated. The residue was purified by flash chromatography (30% EA in PE) to give 2-(1,1-dimethyl-2-oxo-ethyl)-2′-fluoro-5′-methoxy-biphenyl-4-carboxylic acid, 6-6, as yellow solid. ¹H NMR (400 MHZ, CDCl₃) δ 9.43 (s, 1H), 8.28 (s, 1H), 8.09-8.06 (d, 1H), 7.28 (d, J=8.0 Hz, 1H), 7.03 (t, 1H), 6.93-6.89 (m, 1H), 6.64-6.62 (m, 1H), 3.80 (s, 3H), 1.413 (s, 3H), 1.407 (s, 3H).

Step 7. To a mixture of 6-6 (1 g, 3.16 mmol) in dry MeOH (60 mL) was added Bestmann reagent (1.23 g, 6.33 mmol) and K₂CO₃ (1.31 g, 9.48 mmol) at r. t. The resulting mixture became clear after stirring for 16 hrs at r. t. Solvent was removed and the residue was diluted with water (50 mL), acidified with 1N HCl until the pH reached 3 to 5, extracted with EA (50 mL×3), dried and concentrated. The residue was purified by flash chromatography (25% EA in PE) to give 2-(1,1-dimethyl-prop-2-ynyl)-2′-fluoro-5′-methoxy-biphenyl-4-carboxylic acid, 6-7, as white solid. ¹H NMR (400 MHZ, CDCl₃) δ 8.58 (s, 1H), 8.01-7.99 (dd, 1H), 7.21 (d, J=8.0 Hz, 1H), 7.00 (t, 1H), 6.90-6.81 (m, 2H), 3.79 (s, 3H), 2.20 (s, 1H), 1.60 (s 3H), 1.54 (s, 3H).

Step 8. To a solution of 6-7 (785 mg, 2.516 mmol) in dry THF (40 mL) was added LAH (192 mg, 5.032 mmol) portion wise at 0° C. under N₂. The resulting mixture was then stirred and slowly heated at 60° C. for 2 hrs. The reaction mixture was quenched by the addition of H₂O (0.192 mL), NaOH (15%, 0.192 mL) and H₂O (0.576 mL) at 0° C. The reaction mixture was filtered and the filter cake was washed with EA. The combined organic phase was dried over MgSO₄ and concentrated. The residue was purified by flash chromatography (30% EA in PE) to give [2-(1,1-dimethyl-prop-2-ynyl)-2′-fluoro-5′-methoxy-biphenyl-4-yl]-methanol, 6-8, as a white gum. ¹H NMR (400 MHZ, CDCl₃) δ 7.85 (s, 1H), 7.30-7.27 (dd, 1H), 7.08 (d, J=7.6 Hz, 1H), 6.97 (t, 1H), 6.86-6.81 (m, 2H), 4.75 (s, 2H), 3.77 (s, 3H), 2.18 (s, 1H), 1.60 (s 3H), 1.54 (s, 3H).

Step 9. To a solution of 6-8 (50.0 mg, 0.17 mmol) in dry DCM (5 mL) was added Intermediate 5 (37.0 mg, 0.17 mmol) and PPh₃ (88.0 mg, 0.34 mmol) under nitrogen atmosphere at 0° C. The mixture was stirred at 0° C. for 10 minutes, followed with addition of DEAD (58.0 mg, 0.34 mmol). The reaction mixture was allowed to warm up to room temperature and stirred overnight. The reaction mixture was quenched with water and extracted with DCM (20 mL×3). The combined organic layers were washed with water (20 mL×2) and brine (20 mL), dried over Na₂SO₄ and concentrated in vacuum. The residue was purified by flash chromatography (EA/PE=0-30%) to give(S)-methyl 3-cyclopropyl-3-(3-((2′-fluoro-5′-methoxy-2-(2-methylbut-3-yn-2-yl)-[1,1′-biphenyl]-4-yl) methoxy)phenyl) propanoate, 6-9, as a colorless oil. MS: m/z 518.1 (M+H₂O).

Step 10. To a solution of 6-9 (12.0 mg, 0.024 mmol) in MeOH/H₂O (5/2 mL) was added NaOH (9.6 mg, 0.240 mmol) at room temperature. The mixture was heated at 40° C. and stirred overnight. The reaction mixture was acidified by 1 M HCl to pH=3. The reaction mixture was extracted with EA (20 mL×3). The combined organic layers were washed with water (20 mL×2) and brine (20 mL), dried over Na₂SO₄ and concentrated in vacuum. The residue was purified by prepHPLC to give(S)-3-cyclopropyl-3-(3-((2′-fluoro-5′-methoxy-2-(2-methylbut-3-yn-2-yl)-[1,1′-biphenyl]-4-yl)methoxy)phenyl) propanoic acid, Intermediate 6, as a white solid. ¹H NMR (400 MHZ, CDCl₃): δ=7.89 (s, 1H), 7.37-7.35 (d, J=7.2 Hz, 1H), 7.26-7.23 (d, 1H), 7.11-7.09 (d, J=6.8 Hz, 1H), 6.98 (t, J=8.8 Hz, 1H), 6.90-6.81 (m, 5H), 5.10 (s, 2H), 3.78 (s, 3H), 2.80-2.77 (m, 2H), 2.40-2.32 (m, 1H), 2.18 (s, 1H), 1.55 (s, 3H), 1.50 (s, 3H), 1.04-1.02 (m, 1H), 0.60-0.58 (m, 1H), 0.44-0.42 (m, 1H), 0.32-0.28 (m, 1H), 0.18-0.14 (m, 1H). MS: m/z 485.3 (M−H)⁺.

### Synthesis of Intermediate 7

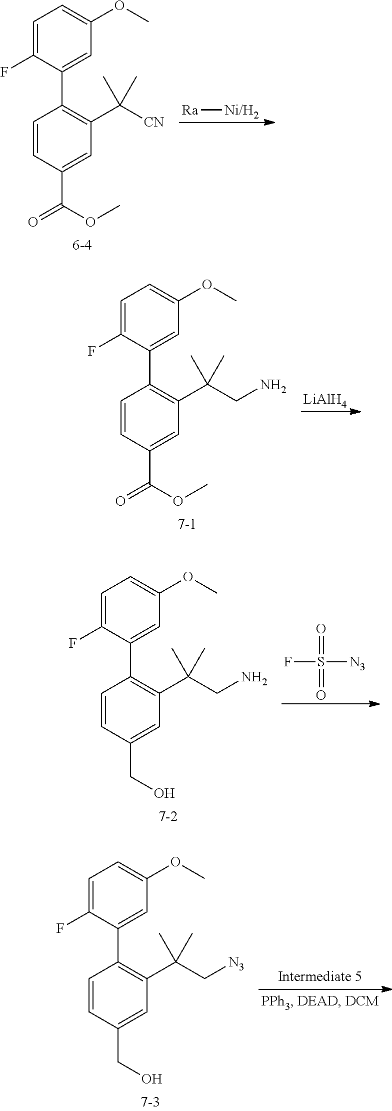

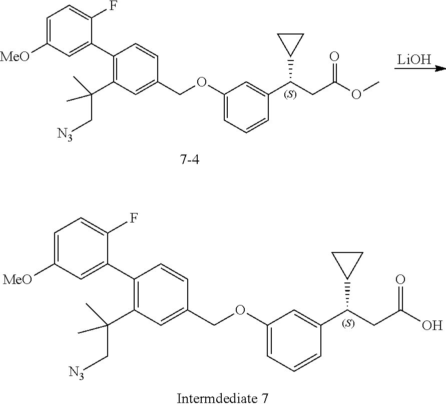

Step 1. A mixture of 2-(cyano-dimethyl-methyl)-2′-fluoro-5′-methoxy-biphenyl-4-carboxylic acid (836 mg, 2.6 mmol) and Raney-Ni (˜200 mg, 20% wt.) in MeOH (30 mL) was degassed and filled with hydrogen using a balloon. The resulting mixture was then hydrogenated for 16 hrs at r. t. Solvent was removed and the residue was purified by flash chromatography (5% MeOH in DCM) to give 2-(2-amino-1,1-dimethyl-ethyl)-2′-fluoro-5′-methoxy-biphenyl-4-carboxylic acid methyl ester 7-1 (780 mg, yield: 93%) as pale-yellow oil. MS (ESI) m/z 332.0 [M+H]⁺.

Step 2. To a mixture of 7-1 (128 mg 26 mmol) in THF (10 mL) at 0° C. under N₂ atmosphere was added LAH (198 mg, 5.2 mmol) in 3 portions. After addition the resulting mixture was stirred for 15 min at 0° C. and stirred for additional 2 hrs at r. t. The reaction mixture was quenched by water (0.2 mL), aq NaOH (15%, 0.2 mL) and water (0.6 mL) at 0° C., diluted with EA (20 mL) and filtered. The filtrate was dried over Na₂SO₄ and concentrated to give crude [2-(2-Amino-1,1-dimethyl-ethyl)-2′-fluoro-5′-methoxy-biphenyl-4-yl]-methanol, 7-2, (110 mg, yield: 95%) as pale-yellow oil. MS (ESI) m/z 304.1 [M+H]⁺.

Step 3. To a mixture 7-2 (420 mg, 1.386 mmol) in DMF (30 mL) at r. t. was added fluorosulfuryl azide (˜0.5 M in MTBE, 2.78 mL) and KHCO₃ (3.0 M, 1.848 mL) dropwise. After addition the resulting mixture was stirred for 4 hrs at r. t. Diluted with water (50 mL), extracted with EA (30 mL×3), dried and concentrated. The residue was purified by flash chromatography (30% EA in PE) to give [2-(2-azido-1,1-dimethyl-ethyl)-2′-fluoro-5′-methoxy-biphenyl-4-yl]-methanol, 7-3, (210 mg, yield: 46% over 2 steps) as white solid. MS (ESI) m/z 325.2 [M-27+Na]⁺.

Step 4. A solution of 7-3 (245 mg, 0.744 mmol), Intermediate 5 (164 mg, 0.744 mmol) and PPh₃ (390 mg, 1.488 mmol) in DCM (15 mL) was degassed and filled with N₂ 3 times and cooled to 0° C. DEAD (260 mg, 1.488 mmol) was then added dropwise via syringe. The resulting mixture was stirred for 12 hrs under N₂, allowing the temperature to slowly warm to r. t. Solvent was removed and the residue was purified by flash chromatography (20% EA in PE) to give (R)-3-{3-[2-(2-Azido-1,1-dimethyl-ethyl)-2′-fluoro-5′-methoxy-biphenyl-4-ylmethoxy]-phenyl}-3-cyclopropyl-propionic acid methyl ester, 7-4, (140 mg, yield: 35%) as white gum. MS (ESI) m/z 532.4 [M+18]⁺.

Step 5. A mixture of 7-4 (140 mg, 0.263 mmol) and LiOH·H₂O (111 mg, 2.63 mmol) in water (5 mL), MeOH (5 mL) and THF (10 mL) was stirred at 50° C. for 14 hrs. MeOH was removed and the residue was acidified with aqueous HCl until pH reached 3 and extracted with EtOAc (15 mL×3). The combined organic layers were dried over MgSO₄ and concentrated. The residue was purified by preparative HPLC (TFA method) to give, Intermediate 7, as white solid. MS (ESI) m/z 535.4 [M+18]⁺. ¹H NMR (400 MHZ, DMSO-d₆) δ 12.01 (br, 1H), 7.64 (s, 1H), 7.38-7.36 (d, 1H), 7.24-7.19 (m, 2H), 7.05-6.99 (m, 2H), 6.96 (s, 1H), 6.91-6.82 (m, 3H), 5.14 (s, 2H), 3.77 (s, 3H), 3.46 (s, 2H), 2.70-2.61 (m, 2H), 2.31-2.25 (m, 1H), 1.23 (s, 3H), 1.15 (s, 3H), 1.04-0.99 (m, 1H), 0.53-0.49 (m, 1H), 0.34-0.23 (m, 2H), 0.13-0.10 (m, 1H).

### Synthesis of Intermediate 8

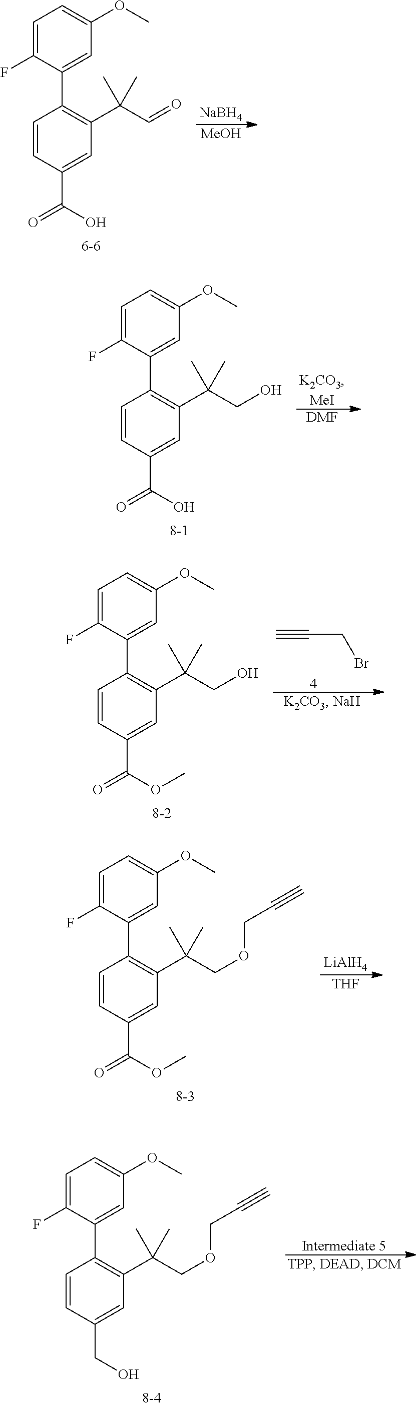

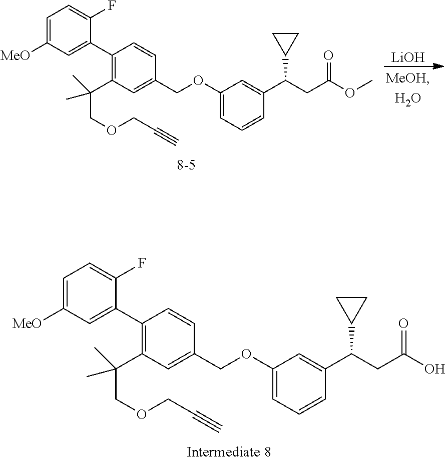

Step 1. To a mixture of 2′-fluoro-5′-methoxy-2-(2-methyl-1-oxopropan-2-yl)-[1,1′-biphenyl]-4-carboxylic acid (3.2 g, 0.010 mol) in MeOH (50 mL) cooled to 0° C. was added NaBH₄ (0.77 g, 0.020 mol) over 15 min. The mixture was stirred at 0° C. for 2 h. The reaction mixture was quenched with NH₄Cl aqueous solution and adjusted to pH=2 with HCl (2N). The resulting mixture was extracted with EA and the organic phase was washed with water and brine, dried with Na₂SO₄, filtered, and the filtrate was concentrated to give 2′-fluoro-2-(1-hydroxy-2-methylpropan-2-yl)-5′-methoxy-[1,l′-biphenyl]-4-carboxylic acid, 8-1, (3.0 g, yield: 93.2%) as a yellow oil.

Step 2. To a solution of 8-1 (3.0 g, 9.4 mmol) in DMF (30 mL) was added K₂CO3 (2.61 g, 18.9 mmol) and MeI (2.68 g, 18.9 mmol). The mixture was stirred at room temperature for 2 h. The reaction mixture was quenched with water and extracted with EA. The organic phase was washed with water and brine, dried with Na₂SO₄, filtered, and the filtrate was concentrated. The residue was purified by column chromatography on silica gel (EA/PE=20%) to give methyl 2′-fluoro-2-(1-hydroxy-2-methylpropan-2-yl)-5′-methoxy-[1,1′-biphenyl]-4-carboxylate, 8-2, (1.5 g, yield: 47.9%) as a yellow solid. ¹H NMR (400 MHz, DMSO-d6): 8.14 (s, 1H), 7.79 (dd, J=1.2 Hz, J=7.6 Hz, 1H), 7.19 (t, J=9.0 Hz, 1H), 7.12 (d, J=7.6 Hz, 1H), 7.01-6.97 (m, 1H), 6.88-6.86 (m, 1H), 4.70 (t, J=5.4 Hz, 1H), 3.88 (s, 3H), 3.75 (s, 3H), 3.47-3.43 (m, 1H), 3.36-3.31 (m, 1H), 1.18 (s, 3H), 1.00 (s, 3H).

Step 3. To a solution 8-2 (1.5 g, 4.5 mmol) in DMF (15 mL) cooled to 0° C. was added NaH (0.36 g, 9.0 mmol). The mixture was stirred at 0° C. for 30 min. Then 3-bromoprop-1-yne (1.08 g, 9.0 mmol) was added and the mixture was stirred at room temperature for 2 h. The reaction mixture was quenched with NH₄Cl aqueous solution and extracted with EA. The organic phase was washed with water and brine, dried with Na₂SO₄, filtered, and the filtrate was concentrated. The residue was purified by column chromatography on silica gel (EA/PE=10%) to give methyl 2′-fluoro-5′-methoxy-2-(2-methyl-1-(prop-2-yn-1-yloxy) propan-2-yl)-[1,1′-biphenyl]-4-carboxylate, 8-3, as a yellow oil.

Step 4. To a solution of methyl 8-3 (450 mg, 1.22 mmol) in THF (10 mL) cooled to 0° C. was added LiAlH₄ (92.4 mg, 2.43 mmol) over 30 min. Then the mixture was stirred at room temperature for 1 h. After the reaction was completed, the reaction mixture was quenched with water (0.1 mL) at 0° C. Then 15% of NaOH aqueous solution (0.1 mL) and water (0.3 mL) were added. The resulting mixture was diluted with EA, dried with MgSO₄, filtered, and the filtrate was concentrated to give (2′-fluoro-5′-methoxy-2-(2-methyl-1-(prop-2-yn-1-yloxy) propan-2-yl)-[1,1′-biphenyl]-4-yl) methanol, 8-4, as a colorless oil. ¹H NMR (400 MHZ, DMSO-d₆): 7.45 (s, 1H), 7.19-7.13 (m, 2H), 6.98-6.94 (m, 1H), 6.91 (d, J=10.8 Hz, 1H), 6.78-6.76 (m, 1H), 5.19 (t, J=5.8 Hz, 1H), 4.52 (d, J=6.0 Hz, 2H), 4.01 (d, J=2.0 Hz, 2H), 3.75 (m, 3H), 3.42-3.35 (m, 3H), 1.18 (s, 3H), 1.07 (s, 3H).

Step 5. To a mixture of 8-4 (300 mg, 0.88 mmol), Intermediate 5 (193.0 mg, 0.88 mmol) and TPP (344.7 mg, 1.32 mmol) in DCM (5 mL) was added DEAD (228.9 mg, 1.32 mmol) at 0° C., and the mixture was stirred at room temperature under N₂ for 12 h. The reaction mixture was quenched with water and extracted with DCM. The organic phase was washed with water and brine, dried with Na₂SO₄, filtered, and the filtrate was concentrated. The residue was purified by column chromatography on silica gel (EA/PE=10%) to give(S) -methyl 3-cyclopropyl-3-(3-((2′-fluoro-5′-methoxy-2-(2-methyl-1-(prop-2-yn-1-yloxy) propan-2-yl)-[1,1′-biphenyl]-4-yl)methoxy)phenyl) propanoate, 8-5, as a colorless oil.

Step 6. To a solution of 8-5 (310 mg, 0.57 mmol) in MeOH (5 mL) and H₂O (0.5 mL) was added LiOH (239.3 mg, 5.70 mmol), and the mixture was stirred at 45° C. for 4 h. After the reaction was completed, the reaction mixture was diluted with water and adjusted to pH=3 with HCl (2N). The resulting mixture was extracted with EA and the organic phase was washed with water and brine, dried with Na₂SO₄, filtered, and the filtrate was concentrated to give(S)-3-cyclopropyl-3-(3-((2′-fluoro-5′-methoxy-2-(2-methyl-1-(prop-2-yn-1-yloxy) propan-2-yl)-[1,1′-biphenyl]-4-yl)methoxy)phenyl) propanoic acid, Intermediate H8, as a white solid. ¹H NMR (400 MHZ, DMSO-d₆): 11.96 (s, 1H), 7.60 (s, 1H), 7.33-7.31 (d, J=8.0 Hz, 1H), 7.23-7.14 (m, 2H), 7.00-6.94 (m, 3H), 6.89-6.79 (m, 3H), 5.11 (s, 2H), 4.01 (s, 2H), 3.75 (s, 3H), 3.45-3.31 (m, 3H), 2.69-2.63 (m, 2H), 2.29-2.23 (m, 1H), 1.19 (s, 3H), 1.08 (s, 3H), 1.02-0.98 (m, 1H), 0.52-0.47 (m, 1H), 0.32-0.22 (m, 2H), 0.13-0.09 (m, 1H). MS (ESI) m/z 529.5 [M−H]⁻.

### Synthesis of Intermediate 9

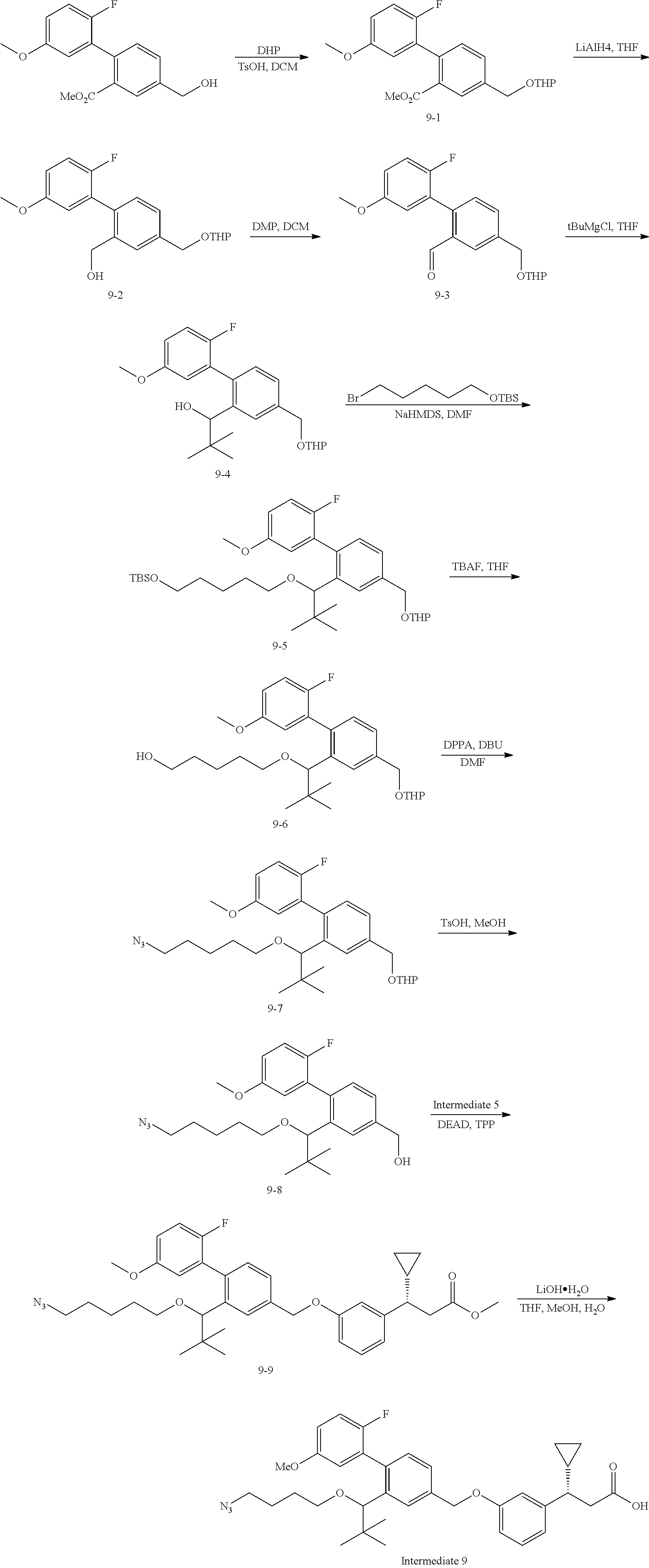

Step 1. To a solution of methyl 2′-fluoro-4-(hydroxymethyl)-5′-methoxy-[1,1′-biphenyl]-2-carboxylate (13.5 g, 0.047 mol) in DCM (200 mL) cooled to 0° C. was added DHP (7.82 g, 0.093 mol) and TsOH (1.77 g, 0.0093 mol). The mixture was stirred at room temperature for 5 h. The reaction mixture was quenched with water and extracted with DCM. The organic phase was washed with water and brine, dried with Na₂SO₄, filtered, and the filtrate was concentrated. The residue was purified by column chromatography on silica gel (EA/PE=10%) to give methyl 2′-fluoro-5′-methoxy-4-(((tetrahydro-2H-pyran-2-yl)oxy)methyl)-[1,1′-biphenyl]-2-carboxylate, 9-1, as a yellow oil.

Step 2. To a solution of 9-1 (15.5 g, 0.041 mol) in THF (150 mL) cooled to 0° C. was added LiAlH₄ (2.36 g, 0.062 mol) over 30 min. Then the mixture was stirred at room temperature for 1 h. The reaction mixture was quenched with water (2.4 mL) at 0° C., followed by addition of 15% of NaOH aqueous solution (2.4 mL) and water (7.2 mL). The resulting mixture was diluted with EA, dried with MgSO₄, filtered, and the filtrate was concentrated to give (2′-fluoro-5′-methoxy-4-(((tetrahydro-2H-pyran-2-yl)oxy)methyl)-[1,1′-biphenyl]-2-yl) methanol, 9-2, as a yellow oil.

Step 3. To a solution of 9-2 (14.0 g, 0.041 mol) in DCM (200 mL) was added Dess-Martin Periodinane (25.7 g, 0.061 mol), and the mixture was stirred at room temperature for 2 h. After the reaction was completed, the reaction mixture was concentrated and the residue was purified by column chromatography on silica gel (EA/PE=20%) to give 2′-fluoro-5′-methoxy-4-(((tetrahydro-2H-pyran-2-yl)oxy)methyl)-[1,l′-biphenyl]-2-carbaldehyde, 9-3, as a yellow oil. ¹H NMR (400 MHZ, DMSO-d₆): 9.84 (d, J=3.6 Hz, 1H), 7.91 (d, J=1.6 Hz, 1H), 7.75 (dd, J=2.0 Hz, J=8.0 Hz, 1H), 7.51 (d, J=7.6 Hz, 1H), 7.27 (t, J=9.2 Hz, 1H), 7.08-7.00 (m,; 2H), 4.82-4.74 (m, 2H), 4.60 (d, J=12.4 Hz, 1H), 3.84-3.80 (m, 4H)3.5; 4-3.49 (m, 1H)_{1.78}-1.67 (m, 2H), 1.59-1.48 (m, 4H).

Step 4. To a solution of 9-3 (5 g, 0.015 mol) in THF (50 mL) cooled to −70° C. was added tBuMgCl (21.4 mL, 0.036 mol) over 20 min. Then the mixture was allowed to warm to room temperature and stirred under N₂ for 12 h. The reaction mixture was quenched with NH₄Cl aqueous solution and extracted with EA. The organic phase was washed with water and brine, dried with Na₂SO₄, filtered, and the filtrate was concentrated. The residue was purified by column chromatography on silica gel (EA/PE=20%) to give 1-(2′-fluoro-5′-methoxy-4-(((tetrahydro-2H-pyran-2-yl)oxy)methyl)-[1,l′-biphenyl]-2-yl)-2,2-dimethylpropan-1-ol, 9-4, as a yellow oil.

Step 5. To a solution of 9-4 (1 g, 2.49 mmol) in DMF (10 mL) cooled to 0° C. was added NaHMDS (5.0 mL, 9.95 mmol) over 10 min under N₂. The mixture was stirred at room temperature for 2 h. Then the reaction mixture was quenched with NH₄Cl aqueous solution and extracted with EA. The organic phase was washed with water and brine, dried with Na₂SO₄, filtered, and the filtrate was concentrated. The residue was purified by column chromatography on silica gel (EA/PE=10%) to give tert-butyl ((5-(1-(2′-fluoro-5′-methoxy-4-(((tetrahydro-2H-pyran-2-yl)oxy)methyl)-[1,1′-biphenyl]-2-yl)-2,2-dimethylpropoxy) pentyl)oxy)dimethylsilane, 9-5, as a yellow oil.

Step 6. To a solution of 9-5 (1 g, 1.66 mmol) in THF (10 mL) cooled to 0° C. was added TBAF (4.98 mL, 4.98 mmol), and the mixture was stirred at room temperature for 2 h. After the reaction was completed, the reaction mixture was quenched with water and extracted with EA. The organic phase was washed with water and brine, dried with Na₂SO₄, filtered, and the filtrate was concentrated. The residue was purified by column chromatography on silica gel (EA/PE=20%) to give 5-(1-(2′-fluoro-5′-methoxy-4-(((tetrahydro-2H-pyran-2-yl)oxy)methyl)-[1,1′-biphenyl]-2-yl)-2,2-dimethylpropoxy) pentan-1-ol, 9-6, as a colorless oil.

Step 7. To a solution of 9-6 (420 mg, 0.86 mmol) in DMF (5 mL) was added DBU (261.6 mg, 1.72 mmol) and DPPA (473.4 mg, 1.72 mmol), and the mixture was stirred at 90° C. for 2 h. After the reaction was completed, the reaction mixture was quenched with water and extracted with EA. The organic phase was washed with water and brine, dried with Na₂SO₄, filtered, and the filtrate was concentrated. The residue was purified by column chromatography on silica gel (EA/PE=10%) to give 2-((2-(1-((5-azidopentyl)oxy)-2,2-dimethylpropyl)-2′-fluoro-5′-methoxy-[1,l′-biphenyl]-4-yl)methoxy)tetrahydro-2H-pyran, 9-7, as a colorless oil.

Step 8. To a solution of 9-7 (100 mg, 0.19 mmol) in MeOH (2 mL) was added TsOH (111.1 mg, 0.58 mmol), and the mixture was stirred at room temperature for 2 h. After the reaction was completed, the reaction mixture was quenched with water and extracted with EA. The organic phase was washed with water and brine, dried with Na₂SO₄, filtered, and the filtrate was concentrated. The residue was purified by column chromatography on silica gel (EA/PE=25%) to give (2-(1-((5-azidopentyl)oxy)-2,2-dimethylpropyl)-2′-fluoro-5′-methoxy-[1,1′-biphenyl]-4-yl) methanol, 9-8, as a yellow oil. ¹H NMR (400 MHZ, DMSO-d₆): 7.45 (d, J=16.4 Hz, 1H), 7.32-7.11 (m, 3H), 6.99-6.95 (m, 1H), 6.77-6.71 (m, 1H), 5.23 (t, J=5.6 Hz, 1H), 4.55 (d, J=5.2 Hz, 2H), 4.19-3.95 (m, 1H), 3.75 (s, 3H), 3.42-3.19 (m, 4H), 1.55-1.40 (m, 6H), 0.66 (s, 9H).

Step 9. To a mixture of 9-8 (60 mg, 0.14 mmol), Intermediate 5 (36.9 mg, 0.17 mmol) and TPP (73.3 mg, 0.28 mmol) in DCM (1 mL) was added DEAD (48.7 mg, 0.28 mmol) at 0° C., and the mixture was stirred at room temperature for 12 h. The reaction mixture was quenched with water and extracted with DCM. The organic phase was washed with water and brine, dried with Na₂SO₄, filtered, and the filtrate was concentrated. The residue was purified by column chromatography on silica gel (EA/PE=10%) to give (3S)-methyl 3-(3-((2-(1-((5-azidopentyl)oxy)-2,2-dimethylpropyl)-2′-fluoro-5′-methoxy-[1,1′-biphenyl]-4-yl)methoxy)phenyl)-3-cyclopropylpropanoate, 9-9,as a colorless gum. ¹H NMR (400 MHZ, CDCl₃): 7.60 (d, J=12.0 Hz, 1H), 7.41 (t, J=8.2 Hz, 1H), 7.25-7.16 (m, 2H), 7.07-6.99 (m, 1H), 6.87-6.83 (m, 4H), 6.74-6.69 (m, 1H), 5.11 (s, 2H), 4.24-4.01 (s, 1H), 3.78 (s, 3H), 3.61 (s, 3H), 3.43-3.38 (m, 1H), 3.30-3.25 (m, 3H), 2.79-2.68 (m, 2H), 2.38-2.32 (m, 1H), 1.64-1.44 (m, 4H), 1.03-0.98 (m, 1H), 0.70 (s, 9H), 0.59-0.56 (m, 1H), 0.43-0.39 (m, 1H); 0.27-0.23 (m, 1H), 0.15-0.12 (m, 1H).

Step 10. To a solution of 9-9 (50 mg, 0.079 mmol) in MeOH (2 mL) and H₂O (0.5 mL) was added LiOH (33.3 mg, 0.792 mmol), and the mixture was stirred at 50° C. for 5 h. After the reaction was completed, the reaction mixture was concentrated. The residue was purified by pre-HPLC to give (3S)-3-(3-((2-(1-(4-azidobutoxy)-2,2-dimethylpropyl)-2′-fluoro-5′-methoxy-[1,1′-biphenyl]-4-yl)methoxy)phenyl)-3-cyclopropylpropanoic acid, Intermediate 9, as a white solid. ¹H NMR (400 MHZ, DMSO-d₆): 11.96 (s, 1H), 7.58 (d, 1H), 7.46-7.41 (m, 1H), 7.27-7.17 (m, 3H), 7.00-6.96 (m, 1H), 6.89 (s, 1H), 6.86-6.83 (m, 2H), 6.80-6.73 (m, 1H), 5.17 (s, 2H), 4.20-3.96 (s, s, two isomers, 1H), 3.75 (s, 3H), 3.38-3.19 (m, 4H), 2.68-2.57 (m, 2H), 2.28-2.22 (m, 1H), 1.53-1.37 (m, 6H), 1.00-0.96 (m, 1H), 0.64 (s, 9H), 0.50-0.45 (m, 1H), 0.30-0.19 (m, 2H), 0.09-0.06 (m, 1H). MS (ESI) m/z 616.5 [M−H]⁻.

### Synthesis of Intermediate 10

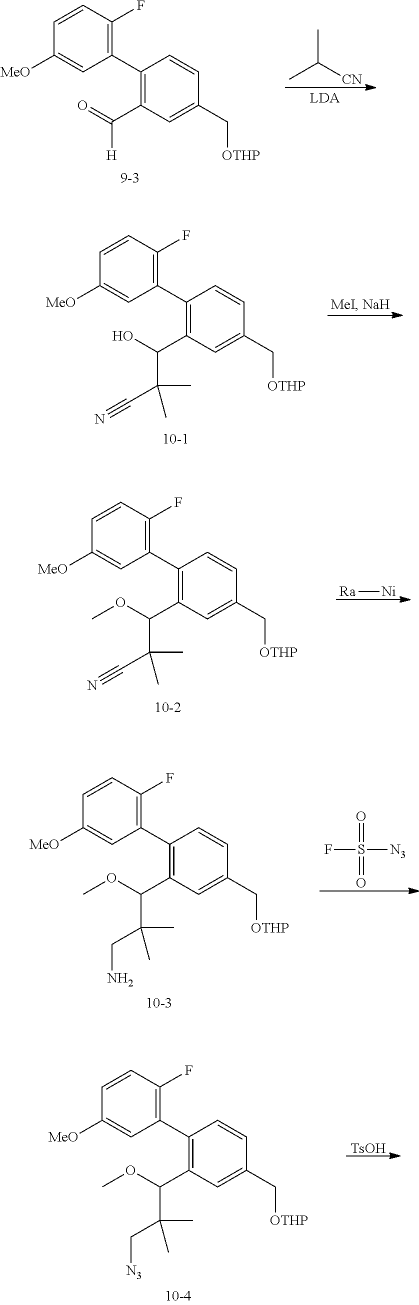

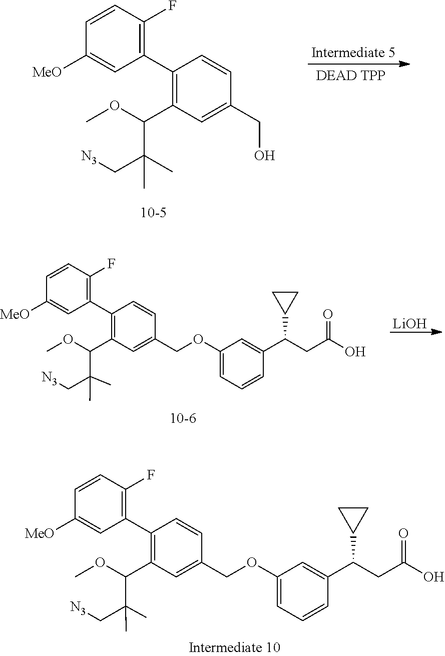 [SMILES: COc1ccc(F)c(-c2ccc(CO)cc2C(OC)C(C)(C)CN=[N+]=[N-])c1 ; COc1ccc(F)c(-c2ccc(COc3cccc([C@@H](CC(=O)O)C4CC4)c3)cc2C(OC)C(C)(C)CN=[N+]=[N-])c1 ; "LiOH" ; COc1ccc(F)c(-c2ccc(COc3cccc([C@@H](CC(=O)O)C4CC4)c3)cc2C(OC)C(C)(C)CN=[N+]=[N-])c1]

Step 1. To a solution of isobutyronitrile (281 mg, 4.07 mmol) in dry THF (15 mL) at −78° C. was added LDA (2.0 M in hexane, 2.14 mL) dropwise over 20 min. The resulting mixture was stirred for 2 hrs at −78° C. Then a mixture of 2′-fluoro-5′-methoxy-4-(tetrahydro-pyran-2-yloxymethyl)-biphenyl-2-carbaldehyde 9-3 in THF (15 mL) was added dropwise over 20 min. The reaction mixture was then stirred for 16 hrs, allowing the temperature to slowly warm to r. t. The mixture was quenched by aq. NH₄Cl at 0° C. and extracted with EA (30 mL×3), dried and concentrated. The residue was purified by flash chromatograph (20% EA in PE) to give 3-[2′-fluoro-5′-methoxy-4-(tetrahydro-pyran-2-yloxymethyl)-biphenyl-2-yl]-3-hydroxy-2,2-dimethyl-propionitrile, 10-1, as a yellow solid. ¹H NMR (400 MHZ, DMSO-d₆) δ 7.88-7.79 (m, 1H), 7.37-7.33 (m, 1H), 7.27-7.17 (m, 2H), 7.10-6.97 (m, 1H), 6.90-6.84 (m, 1H), 6.17-6.15 (m, 1H), 4.75-4.72 (m, 2H), 4.54-4.36 (m, 2H), 3.85-3.80 (m, 1H), 3.76-3.75 (m, 3H), 3.52-3.48 (m, 1H), 1.77-1.64 (m, 2H), 1.57-1.44 (m, 4H), 1.25-1.15 (m, 6H).

Step 2. To a mixture of 10-1 (500 mg, 1.453 mmol) in DMF (8 mL) at 0° C. was added NaH (116 mg, 60%, 2.905 mmol) in portions under N₂. The resulting mixture was stirred for 30 min at 0° C. Then Mel (412 mg, 2.905 mmol) was added. Stirred for additional 2 hrs at r. t. The reaction mixture was quenched with aq. NH₄Cl at 0° C. and extracted with EA (40 mL×3), dried and concentrated. The residue was purified by flash chromatograph (10% EA in PE) to give 3-[2′-fluoro-5′-methoxy-4-(tetrahydro-pyran-2-yloxymethyl)-biphenyl-2-yl]-3-methoxy-2,2-dimethyl-propionitrile, 10-2, as a colorless oil.

Step 3. A mixture of 10-2 (311 mg, 0.7 mmol) and Raney-Ni (˜60 mg, 20% wt) in MeOH (30 mL) was hydrogenated with a balloon at 25° C. for 16 hrs. The mixture was filtered over celite and concentrated. The residue was purified by flash chromatograph (5% to 10% of MeOH in DCM) to give 3-[2′-fluoro-5′-methoxy-4-(tetrahydro-pyran-2-yloxymethyl)-biphenyl-2-yl]-3-methoxy-2,2-dimethyl-propylamine, 10-3, as a pale-yellow oil. MS (ESI) m/z 432.1 [M+H]⁺.

Step 4. To a mixture of 10-3 (280 mg, 0.648 mmol) in DMF (30 mL) at r. t. was added fluorosulfuryl azide (˜0.5 M in MTBE, 1.4 mL) and KHCO₃ (3.0 M, 0.9 mL) dropwise. After addition the resulting mixture was stirred for 4 hrs at r. t. The mixture was diluted with water (50 mL), extracted with EA (30 mL×3), dried and concentrated. The residue was purified by flash chromatography (20% EA in PE) to give 2-[2-(3-azido-1-methoxy-2,2-dimethyl-propyl)-2′-fluoro-5′-methoxy-biphenyl-4-ylmethoxy]-tetrahydro-pyran, 10-4, as a yellow oil.

Step 5. To a mixture of 10-4 (300 mg, 0.656 mmol) in MeOH (6 mL) was added TsOH·H₂O (374 mg, 1.969 mmol). The resulting mixture was stirred for 2 hrs at r. t. The reaction mixture was diluted with water (20 mL), extracted with EA (30 mL×4) and concentrated. The residue was purified by flash chromatography (25% EA in PE) to give [2-(3-azido-1-methoxy-2,2-dimethyl-propyl)-2′-fluoro-5′-methoxy-biphenyl-4-yl]-methanol, 10-5, as a yellow oil.

Step 6. A solution of 10-5 (210 mg, 0.563 mmol), Intermediate 5 (160 mg, 0.76 mmol) and PPh₃ (295 mg, 1.126 mmol) in DCM (8 mL) was degassed and filled with N₂ 3 times and cooled to 0° C. DEAD (196 mg, 1.126 mmol) was then added dropwise via syringe. The resulting mixture was then stirred for 12 hrs under N₂, left the temperature slowly warm to r. t. Solvent was removed and the residue was purified by flash chromatography (10% EA in PE) to give 3-{3-[2-(3-azido-1-methoxy-2,2-dimethyl-propyl)-2′-fluoro-5′-methoxy-biphenyl-4-ylmethoxy]-phenyl}-3-cyclopropyl-propionic acid methyl ester, 10-6, as a colorless oil. Note: weak MS. ¹H NMR (400 MHZ, DMSO-d₆) δ 7.55-7.48 (m, 2H), 7.25-7.17 (m, 3H), 7.02-6.99 (m, 1H), 6.91 (s, 1H), 6.86-6.81 (m, 3H), 5.20 (s, 2H), 4.41-4.16 (m, 1H), 3.75 (s, 3H), 3.51 (s, 3H), 3.35-3.32 (m, 1H), 3.22-3.14 (m, 3H), 2.95-2.92 (m, 1H), 2.76-2.68 (m, 2H), 2.26-2.22 (m, 1H), 1.01-0.98 (m, 1H), 0.65 (s, 3H), 0.49-0.46 (m, 1H), 0.38 (s, 3H), 0.29-0.26 (m, 1H), 0.20-0.15 (m, 1H), 0.10-0.05 (m, 1H).

Step 7. A mixture of 10-6 (180 mg, 0.31 mmol) and LiOH·H₂O (128 mg, 3.13 mmol) in water (3 mL), MeOH (3 mL) and THF (3 mL) was stirred at 55° C. for 14 hrs. MeOH and THF were removed and the residue was acidified with aqueous HCl until pH reached 3 and extracted with EtOAc (15 mL×3). The combined organic layers were dried over Na₂SO₄ and concentrated to give crude (3S)-3-(3-((2-(3-azido-1-methoxy-2,2-dimethylpropyl)-2′-fluoro-5′-methoxy-[1,l′-biphenyl]-4-yl)methoxy)phenyl)-3-cyclopropylpropanoic acid, Intermediate 10, as a yellow oil, which was purified with preparative HPLC to give a while solid. MS (ESI) m/z 560.4 [M−H]⁻. ¹H NMR (400 MHZ, DMSO-d₆): 11.98 (br, 1H), 7.56-7.48 (m, 2H), 7.27-7.17 (m, 3H), 7.02-6.99 (m, 1H), 6.90-6.82 (m, 4H), 5.20 (s, 2H), 4.42, 4.16 (s, s, isomers, 1H), 3.75 (s, 3H), 3.36-3.30 (d, J=23 Hz, 1H), 3.23, 3.15 (s, s, isomers, 3H), 2.96-2.93 (d, J=23 Hz, 1H), 2.65-2.61 (m, 2H), 2.26-2.22 (m, 1H), 1.00-0.97 (m, 1H), 0.67, 0.38 (s, s, isomers, 6H), 0.50-0.45 (m, 1H), 0.29-0.20 (m, 2H), 0.09-0.07 (m, 1H).

### Synthesis of Intermediate 11

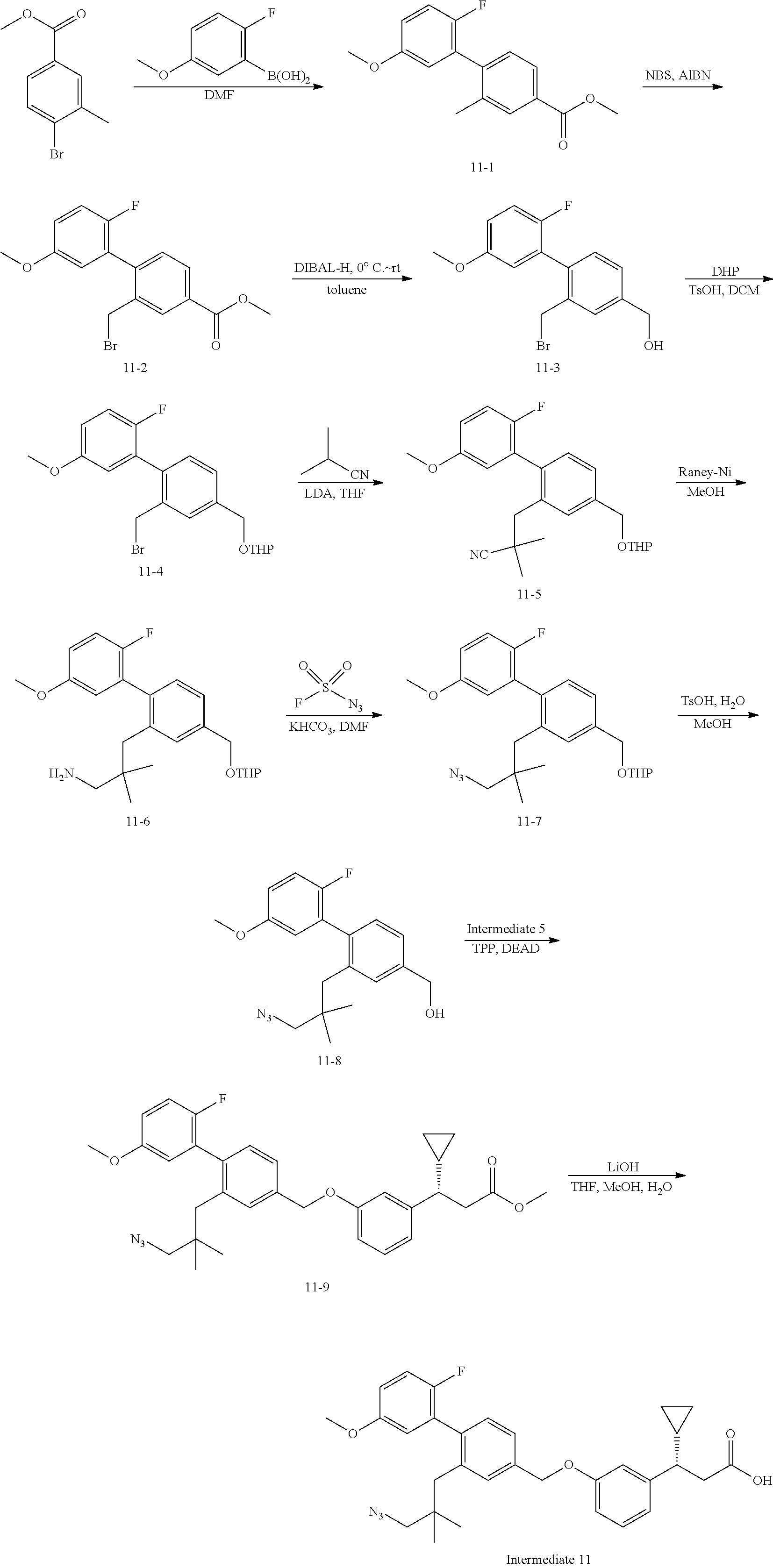

Step 1. A mixture of 4-bromo-3-methyl-benzoic acid methyl ester (19 g, 83.2 mol), phenylboronic acid 2 (21.2 g, 124 mol), (PPh₃)₄ (4.8 g, 4.16 mmol) and K₂CO₃ (22.96 g, 166.4 mol) in DMF (200 mL) was degassed and filled with N₂ 3 times and heated at 100° C. for 16 hrs. DMF was removed and the residue was diluted with water (100 mL), extracted with EA (60 mL×3), dried and concentrated. The residue was purified by flash chromatography (10% of EA in PE) to give 2-bromomethyl-2′-fluoro-5′-methoxy-biphenyl-4-carboxylic acid methyl ester, 11-1, (20 g, yield: 88%) as a red oil. MS (ESI) m/z 275.0 [M+H]⁺.

Step 2. To a mixture of 11-1 (20 g, 72.99 mol) and AIBN(2.38 g, 14.52 mol) in CCl₄ (250 mL) at r. t. was added NBS (10.3 g, 58.08 mol) in small portions. The resulting mixture was heated to reflux (85° C.) for 20 hrs. Solvent was removed and the residue was purified by flash chromatography (5% to 10% of EA in PE) to give 2-bromomethyl-2′-fluoro-5′-methoxy-biphenyl-4-carboxylic acid methyl ester, 11-2, as yellow oil. MS (ESI) m/z 352.9 [M+H]⁺.

Step 3. To a mixture of 11-2 (27 g, about 73 mol) in toluene (300 mL) at 0° C. was added DIBAL-H (1.0 M in hexane, 150 mL) dropwise over 30 min. The resulting mixture was stirred for 16 hrs, allowing the temperature to slowly warm to r. t. The mixture was quenched with aq. NH₄Cl at 0° C. and the precipitate was removed by filtration. Organic phase was separated and the water phase was extracted with EA (100 mL×3). The organic phase was combined, dried and concentrated to give crude (2-bromomethyl-2′-fluoro-5′-methoxy-biphenyl-4-yl)-methanol, 11-3, as orange oil. MS (ESI) m/z 342.1 [M+18]⁺.

Step 4. To a mixture of 11-3 (13.6 g, 41.9 mmol) and TsOH·H₂O (796 mg, 4.19 mmol) in DCM (150 mL) at r. t. was added DHP (5.28 g, 62.85 mmol) dropwise. The mixture was stirred at room temperature for 12 h, and quenched with water (100 mL). The organic layer was separated, extracted with DCM (50 mL×3). The organic phase was combined, dried and concentrated. The residue was purified by flash chromatography (5% to 10% of EA in PE) to give 2-(2-bromomethyl-2′-fluoro-5′-methoxy-biphenyl-4-ylmethoxy)-tetrahydro-pyran, 11-4, as a white oil.

Step 5. To a solution of isobutyronitrile (5.06 g, 0.073 mol) in THF (100 mL) cooled to −70° C. was added LDA (36.7 mL, 0.073 mol) dropwise under N₂ and the mixture was stirred at −70° C. for 2 h. Then a solution of 11-4 (15.0 g, 0.037 mmol) in THF (50 mL) was added at −70° C. and the mixture was stirred at room temperature for 12 h. The reaction mixture was quenched with NH₄Cl aqueous solution and extracted with EA. The organic phase was washed with water and brine, dried with Na₂SO₄, filtered, and the filtrate was concentrated. The residue was purified by column chromatography on silica gel (EA/PE=25%) to give 3-(2′-fluoro-5′-methoxy-4-(((tetrahydro-2H-pyran-2-yl)oxy)methyl)-[1,1′-biphenyl]-2-yl)-2,2-dimethylpropanenitrile, 11-5, as a yellow solid. ¹H NMR (400 MHZ, DMSO-d₆): 7.51 (s, 1H), 7.35 (d, J=8.0 Hz, 1H), 7.25-7.19 (m, 2H), 7.01-6.98 (m, 1H), 6.88-6.86 (m, 1H), 4.72 (dd, J=4.4 Hz, J=7.6 Hz, 1H), 4.51 (d, J=12.4 Hz, 1H), 3.85-3.79 (m, 1H), 3.75 (s, 3H), 3.52-3.48 (m, 1H), 2.96-2.93 (m, 1H), 2.75-2.72 (m, 1H), 1.80-1.65 (m, 2H), 1.58-1.48 (m, 4H), 1.12 (s, 3H), 1.03 (s, 3H).

Step 6. To a solution of 11-5 (5.0 g, 12.6 mmol) in MeOH (150 mL) was added Raney Ni (5 g) and the mixture was stirred at 45° C. under H₂ for 12 h. After the reaction was completed, the reaction mixture was filtered, and the filtrate was concentrated to give 3-(2′-fluoro-5′-methoxy-4-(((tetrahydro-2H-pyran-2-yl)oxy)methyl)-[1,1′-biphenyl]-2-yl)-2,2-dimethyl propan-1-amine, 11-6, as a yellow oil. MS (ESI) m/z 402.3 [M+H]⁺.

Step 7. To a solution of 11-6 (2 g, 4.99 mmol) in DMF (10 mL) cooled to 0° C. was added KHCO₃ aqueous solution (6.65 mL, 19.95 mmol) and sulfurazidic fluoride (11.0 mL, 5.49 mmol) in MTBE. The mixture was stirred at room temperature for 1 h. The reaction mixture was quenched with water and extracted with EA. The organic phase was washed with water and brine, dried with Na₂SO₄, filtered, and the filtrate was concentrated. The residue was purified by column chromatography on silica gel (EA/PE=10%) to give 2-((2-(3-azido-2,2-dimethylpropyl)-2′-fluoro-5′-methoxy-[1,1′-biphenyl]-4-yl)methoxy)tetrahydro-2H-pyran, 11-7, as a yellow oil. MS (ESI) m/z 450.2 [M+Na]⁺.

Step 8. To a solution of 11-7 (2.0 g, 4.68 mmol) in MeOH (20 mL) was added TsOH (1.78 g, 9.37 mmol) and the mixture was stirred at room temperature for 1 h. The reaction mixture was diluted with water and extracted with EA. The organic phase was washed with water and brine, dried with Na₂SO₄, filtered, and the filtrate was concentrated. The residue was purified by column chromatography on silica gel (EA/PE=20%) to give (2-(3-azido-2,2-dimethyl propyl)-2′-fluoro-5′-methoxy-[1,1′-biphenyl]-4-yl) methanol, 11-8, as a yellow oil. ¹H NMR (400 MHZ, DMSO-d₆): 7.26-7.14 (m, 4H), 6.98-6.94 (m, 1H), 6.83-6.80 (m, 1H), 5.22 (t, J=5.8 Hz, 1H), 4.53 (d, J=5.2 Hz, 2H), 3.75 (s, 3H), 3.02 (d, J=3.2 Hz, 2H), 2.66-2.50 (m, 2H), 0.62 (s, 6H). MS (ESI) m/z 366.2 [M+Na]⁺.

Step 9. To a mixture of 11-8 (1.4 g, 4.08 mmol), Intermediate 5 (1.35 g, 6.12 mmol) and TPP (1.60 g, 6.12 mmol) in DCM (15 mL) cooled to 0° C. was added DEAD (1.07 g, 6.12 mmol) under N₂. The mixture was stirred at room temperature for 12 h. The reaction mixture was quenched with water and extracted with DCM. The organic phase was washed with water and brine, dried with Na₂SO₄, filtered, and the filtrate was concentrated. The residue was purified by column chromatography on silica gel (EA/PE=10%) to give(S)-methyl 3-(3-((2-(3-azido-2,2-dimethylpropyl)-2′-fluoro-5′-methoxy-[1,1′-biphenyl]]-4-yl)methoxy)phenyl)-3-cyclopropylpropanoate, 11-9, as a colorless oil. MS (ESI) m/z 546.2 [M+H]⁺.

Step 10. To a solution of 11-9 (1.2 g, 2.20 mmol) in MeOH (5 mL) and THF (5 mL) was added LiOH (0.92 g, 22.02 mmol) and H₂O (1 mL), and the mixture was stirred at 45° C. for 8 h. After the reaction was completed, the reaction mixture was diluted with water and adjusted to pH=5 with HCl (2N). The resulting mixture was extracted with EA and the organic phase was washed with water and brine, dried with Na₂SO₄, filtered, and the filtrate was concentrated to give(S)-3-(3-((2-(3-azido-2,2-dimethylpropyl)-2′-fluoro-5′-methoxy-[1,1′-biphenyl]-4-yl)methoxy)phenyl)-3-cyclopropylpropanoic acid, Intermediate 11, as a white solid. ¹H NMR (400 MHZ, DMSO-d₆): 11.97 (br, 1H), 7.40-7.36 (m, 2H), 7.24-7.18 (m, 3H), 6.99-6.96 (m, 1H), 6.91 (s, 1H), 6.87-6.83 (m, 3H), 5.14 (s, 2H), 3.75 (s, 3H), 2.99 (d, J=2.4 Hz, 2H), 2.69-2.58 (m, 4H), 2.29-2.23 (m, 1H), 1.01-0.97 (m, 1H), 0.59 (s, 6H), 0.52-0.46 (m, 1H), 0.32-0.20 (m, 2H), 0.12-0.08 (m, 1H). MS (ESI) m/z 530.1 [M−H]⁻.

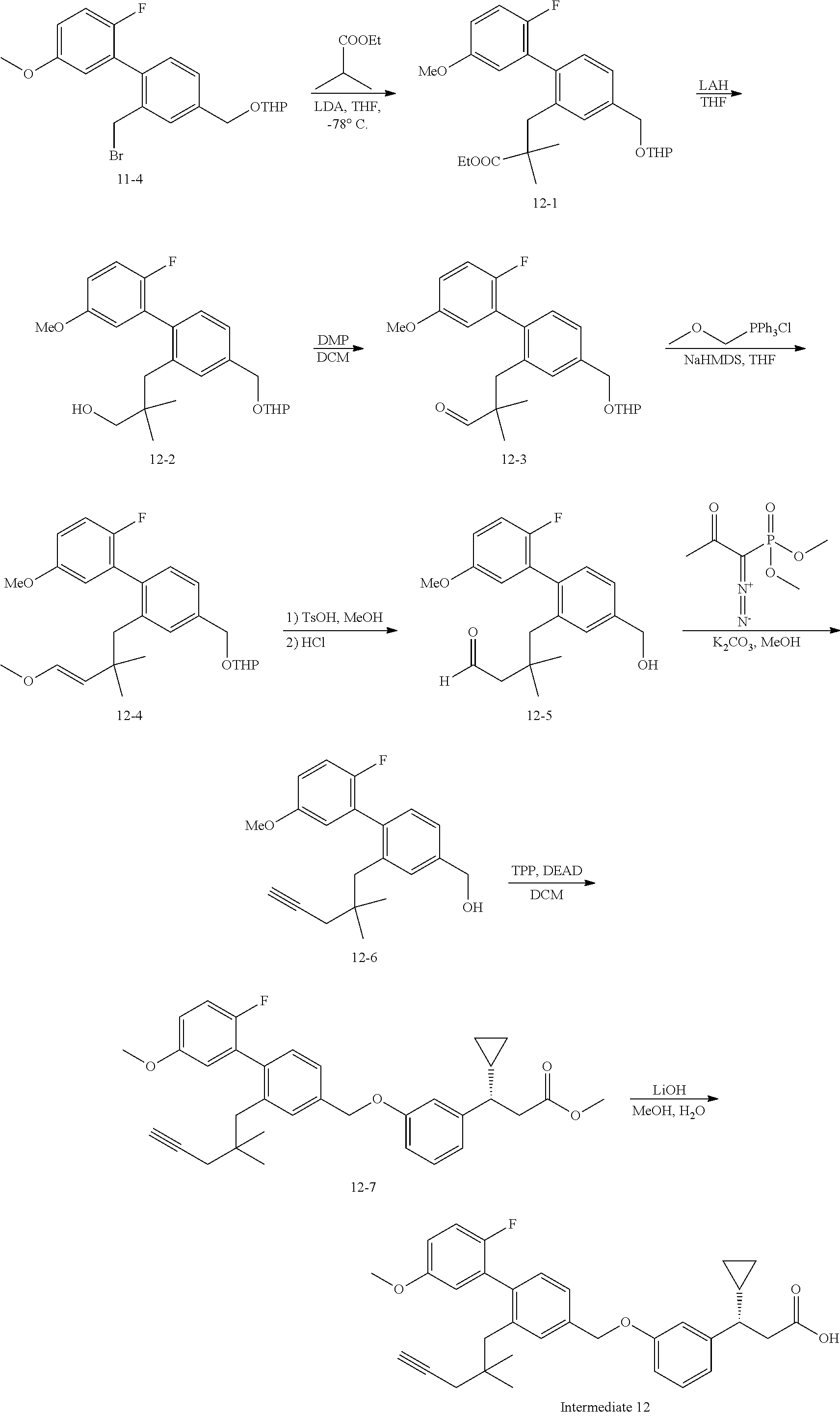

Step 1. To a solution of 2-((2-(bromomethyl)-2′-fluoro-5′-methoxy-[1,1′-biphenyl]-4-yl)methoxy)tetrahydro-2H-pyran (11-4)(5.00 g, 12.22 mmol) in dry THF (50 mL) was added dropwise LDA (18.3 mL, 36.66 mmol, 2 M) at −78° C. under nitrogen atmosphere. The reaction mixture was stirred at −78° C. for 1 hour. Then to the mixture was added dropwise ethyl isobutyrate (2.80 g, 12.22 mmol). The reaction mixture was stirred at −78° C. for 1 hour, and was warmed up to room temperature and stirred overnight. The reaction mixture was quenched with NH₄Cl solution (10 mL). The resulting mixture was extracted with EA (30 mL×3), dried with MgSO₄, filtered, and the filtrate was concentrated. The residue was purified by flash (EA/PE=0-30%) to give ethyl 3-(2′-fluoro-5′-methoxy-4-(((tetrahydro-2H-pyran-2-yl)oxy)methyl)-[1,1′-biphenyl]-2-yl)-2,2-dimethylpropanoate (12-1)(2.0 g, yield: 36.8%) as a colorless oil. MS (ESI) m/z 467.2 [M+Na]⁺.

Step 2. To a solution of 12-1 (1.75 g, 3.94 mmol) in dry THF (20 mL) was added LAH (0.18 g, 4.73 mmol) at 0° C. The reaction mixture was stirred at room temperature for 1 hour. The reaction mixture was quenched with water (0.18 mL+0.54 mL) and sodium hydroxide aqueous solution (0.18 mL, 15%). The resulting mixture was dried with MgSO₄ and filtered. The filtrate was concentrated to give 3-(2′-fluoro-5′-methoxy-4-(((tetrahydro-2H-pyran-2-yl)oxy)methyl)-[1,1′-biphenyl]-2-yl)-2,2-dimethylpropan-1-ol (12-2)(1.6 g) as a yellow oil. MS (ESI) m/z 425.2 [M+Na]⁺.

Step 3. To a solution of 12-2 (0.90 g, 2.24 mmol) in DCM (10 mL) was added Dess-Martin Periodinane (1.42 g, 3.36 mmol), and the mixture was stirred at room temperature for 1 h. After the reaction was completed, the reaction mixture was concentrated and the residue was purified by column chromatography on silica gel (EA/PE=20%) to give 3-(2′-fluoro-5′-methoxy-4-(((tetrahydro-2H-pyran-2-yl)oxy)methyl)-[1,1′-biphenyl]-2-yl)-2,2-dimethylpropanal (12-3)(0.72 g, yield: 80.4%) as a yellow oil. ¹H NMR (400 MHZ, DMSO-d₆): 9.28 (s, 1H), 7.28-7.17 (m, 4H), 7.00-6.97 (m, 1H), 6.80-6.78 (m, 1H), 4.70-4.67 (m, 2H), 4.47 (d, J=12.4 Hz, 1H), 3.83-3.76 (m, 4H), 3.52-3.47 (m, 1H), 1.79-1.64 (m, 2H), 1.56-1.48 (m, 4H).

Step 4. To a solution of chloro(methoxymethyl) triphenylphosphorane (1.29 g, 3.75 mmol) in THF (10 mL) cooled to −70° C. was added NaHMDS (1.9 mL, 3.75 mmol) under N₂, and the mixture was stirred at −70° C. for 30 min. Then 12-3 (1.0 g, 2.50 mmol) in THF (2 mL) was added, and the mixture was allowed to warm to room temperature and stirred for 12 h. After the reaction was completed, the reaction mixture was quenched with NH₄Cl aqueous solution and extracted with EA. The organic phase was washed with water and brine, dried with Na₂SO₄, filtered, and the filtrate was concentrated. The residue was purified by column chromatography on silica gel (EA/PE=20%) to give (E)-2-((2′-fluoro-5′-methoxy-2-(4-methoxy-2,2-dimethylbut-3-en-1-yl)-[1,1′-biphenyl]-4-yl)methoxy)tetrahydro-2H-pyran (12-4)(0.61 g, yield: 57.0%) as a colorless oil. MS (ESI) m/z 451.3 [M+Na]⁺.

Step 5. To a solution of 12-4 (610 mg, 1.43 mmol) in MeOH (10 mL) was added TsOH (1.08 g, 5.70 mmol), and the mixture was stirred at room temperature for 2 h. The reaction mixture was concentrated and dissolved in THF (5 mL). To the mixture was added HCl (2N, 5 mL) and the mixture was stirred at 70° C. for 2 h. After the reaction was completed, the reaction mixture was diluted with water and extracted with EA. The organic phase was washed with water and brine, dried with Na₂SO₄, filtered, and the filtrate was concentrated. The residue was purified by column chromatography on silica gel (EA/PE=25%) to give 4-(2′-fluoro-4-(hydroxymethyl)-5′-methoxy-[1,1′-biphenyl]-2-yl)-3,3-dimethylbutanal (12-5)(380 mg, yield: 80.8%) as a colorless oil. ¹H NMR (400 MHZ, DMSO-d₆): 9.49 (t, J=2.8 Hz, 1H), 7.27-7.14 (m, 4H), 6.98-6.94 (m, 1H), 6.82-6.79 (m, 1H), 5.22-5.18 (m, 1H), 4.52 (t, J=4.8 Hz, 2H), 3.75 (s, 3H), 2.76-2.57 (m, 2H), 2.04 (s, 2H), 0.77 (s, 6H).

Step 6. To a solution of 12-5 (350 mg, 1.06 mmol) in MeOH (200 mL) was added K₂CO₃ (292.7 mg, 2.12 mmol) and dimethyl 1-diazo-2-oxopropylphosphonate (308.6 mg, 1.59 mmol). The mixture was stirred at room temperature for 8 h. The reaction mixture was quenched with water and extracted with EA. The organic phase was washed with water and brine, dried with Na₂SO₄, filtered, and the filtrate was concentrated. The residue was purified by column chromatography on silica gel (EA/PE=25%) to give (2-(2,2-dimethylpent-4-yn-1-yl)-2′-fluoro-5′-methoxy-[1,1′-biphenyl]-4-yl) methanol (12-6)(280 g, yield: 81.0%) as a colorless oil. ¹H NMR (400 MHZ, DMSO-d₆): 7.27-7.14 (m, 4H), 6.97-6.93 (m, 1H), 6.81-6.78 (m, 1H), 5.20 (t, J=5.8 Hz, 1H), 4.53 (d, J=5.6 Hz, 2H), 3.75 (s, 3H), 2.73-2.50 (m, 3H), 1.89 (s, 2H), 0.66 (s, 6H).

Step 7. To a mixture of (2-(2,2-dimethylpent-4-yn-1-yl)-2′-fluoro-5′-methoxy-[1,1′-biphenyl]-4-yl) methanol (12-6)(250 mg, 0.77 mmol), (S)-methyl 3-cyclopropyl-3-(3-hydroxyphenyl) propanoate (253.1 mg, 1.15 mmol) and TPP (401.9 mg, 1.53 mmol) in DCM (5 mL) was added DEAD (266.9 mg, 1.53 mmol) at 0° C., and the mixture was stirred at room temperature for 12 h. After the reaction was completed, the reaction mixture was quenched with water and extracted with DCM. The organic phase was washed with water and brine, dried with Na₂SO₄, filtered, and the filtrate was concentrated. The residue was purified by column chromatography on silica gel (EA/PE=10%) to give(S)-methyl 3-cyclopropyl-3-(3-((2-(2,2-dimethylpent-4-yn-1-yl)-2′-fluoro-5′-methoxy-[1,1′-biphenyl]-4-yl)methoxy)phenyl) propanoate (12-7)(300 mg, yield: 74.1%) as a colorless oil.

Step 8. To a solution of 12-7 (300 mg, 0.57 mmol) in MeOH (5 mL), THF (5 mL) and H₂O (1 mL) was added LiOH (238.6 mg, 5.68 mmol), and the mixture was stirred at 50° C. for 4 h. The reaction mixture was diluted with water and adjusted to pH=4 with HCl (2N). The resulting mixture was extracted with EA. The organic phase was washed with water and brine, dried with Na₂SO₄, filtered, and the filtrate was concentrated to give(S)-3-cyclopropyl-3-(3-((2-(2,2-dimethylpent-4-yn-1-yl)-2′-fluoro-5′-methoxy-[1,l′-biphenyl]-4-yl)methoxy)phenyl) propanoic acid (Intermediate 12)(250 mg, yield: 85.6%) as a white solid. ¹H NMR (400 MHZ, DMSO-d₆): 7.41 (s, 1H), 7.40-7.36 (d, 1H), 7.23-7.16 (m, 3H), 6.98-6.94 (m, 1H), 6.92 (s, 1H), 6.87-6.81 (m, 3H), 5.13 (s, 2H), 3.75 (s, 3H), 2.73 (t, J=2 Hz, 1H), 2.69-2.56 (m, 4H), 2.29-2.23 (m, 1H), 1.87 (brs, 2H), 1.02-0.95 (m, 1H), 0.64 (s, 6H), 0.51-0.46 (m, 1H), 0.32-0.20 (m, 2H), 0.12-0.08 (m, 1H). MS (ESI) m/z 513.1 [M−H]⁻.

### Synthesis of Intermediate 13

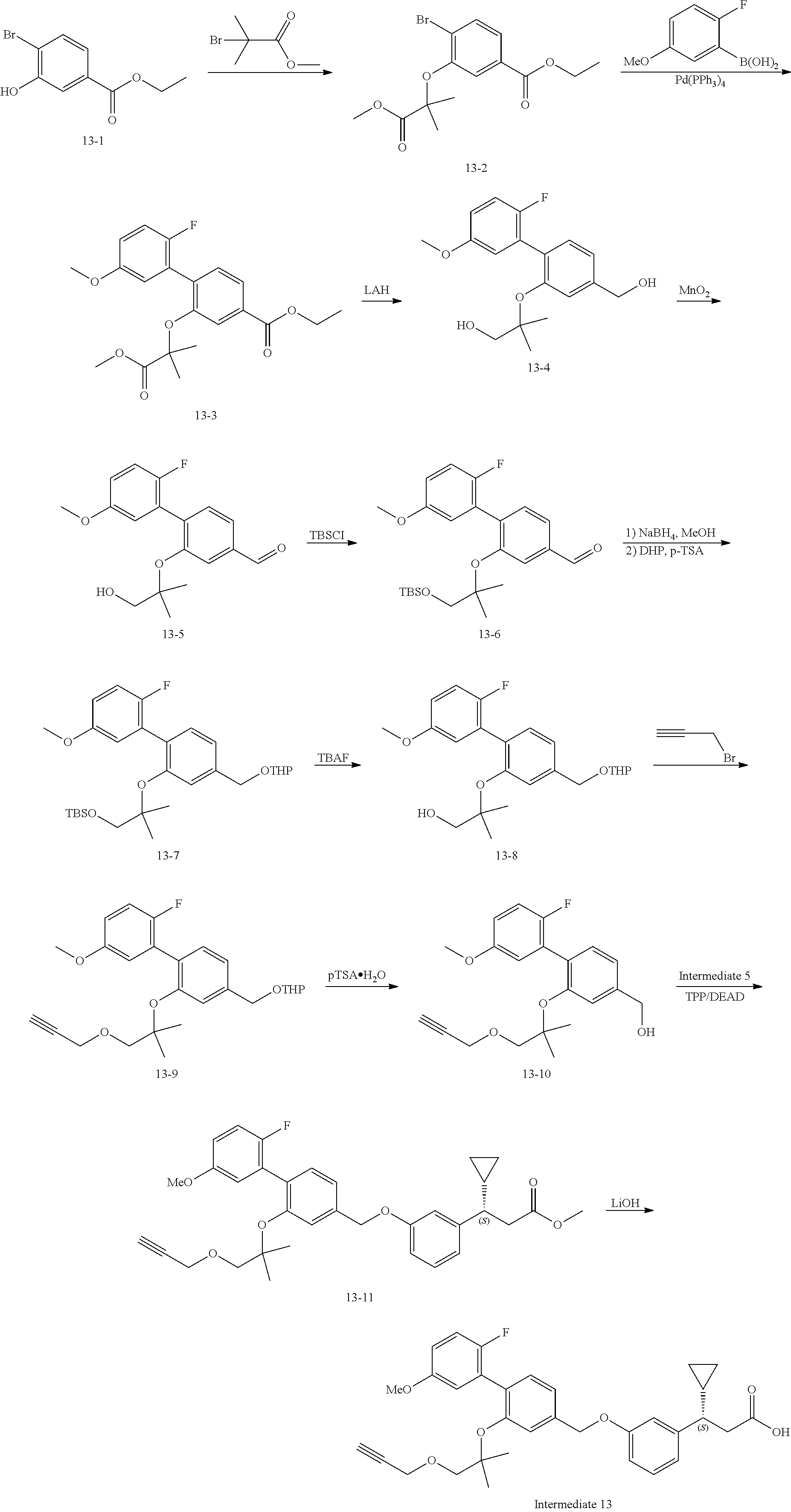

Step 1. A mixture of 4-bromo-3-hydroxy-benzoic acid ethyl ester 13-1 (4.0 g, 16.32 mmol), 2-bromo-2-methyl-propionic acid methyl ester (4.4 g, 24.48 mmol) and Cs2CO3 (10.6 g, 32.64 mmol) in DMF (100 mL) was heated at 105° C. for 16 hrs under N₂ atmosphere. The mixture was diluted with water (150 mL), extracted with EA (150 mL×3), dried and concentrated. The residue was purified by flash chromatography (15% EA to 30% EA in PE) to give 4-bromo-3-(1-methoxycarbonyl-1-methyl-ethoxy)-benzoic acid ethyl ester, 13-2, (3 g, yield: 53%) as yellow gum. MS (ESI) m/z 345.1 [M+H]⁺. ¹H NMR (400 MHZ, DMSO-d₆) δ 7.79 (d, J=8.0 Hz, 1H), 7.53-7.50 (m, 1H), 7.33 (s, 1H), 4.30 (q, J=7.2 Hz, 2H), 3.78 (s, 3H), 1.58 (s, 6H), 1.17 (t, J=7.2 Hz, 3H).

Step 2. A mixture of 13-2 (3 g, 8.69 mmol), 2-fluoro-5-methoxyphenylboronic acid (2.22 g, 13.0 mmol), (PPh₃)₄ (502 mg, 0.434 mmol) and K₂CO₃ (2.41 g, 17.38 mmol) in DMF (80 mL) was degassed and filled with N₂ 3 times and heated at 105° C. for 16 hrs. The mixture was diluted with water (100 mL), extracted with EA (80 mL×3), dried and concentrated. The residue was purified by flash chromatography (5% to 8% of EA in PE) to give 2′-fluoro-5′-methoxy-2-(1-methoxycarbonyl-1-methyl-ethoxy)-biphenyl-4-carboxylic acid ethyl ester 13-3 (2.4 g, yield: 70.8%) as a white solid. MS (ESI) m/z 408.2 [M+18]⁺.

Step 3. To a mixture of 13-3 (1.0 g, 2.56 mmol) in THF (60 mL) at 0° C. under N₂ atmosphere was added LAH (292 mg, 7.69 mmol) in portions. After addition the resulting mixture was stirred for 30 min at r. t. TLC indicated the completion of reaction. The mixture was quenched at 0° C. by water (0.29 mL), aq. NaOH (15%, 0.29 mL), water (0.87 mL) successively and filtered. The filter cake was washed with EA (20 mL×3). The combined organic phase was dried and concentrated to give crude 2-(2′-fluoro-4-hydroxymethyl-5′-methoxy-biphenyl-2-yloxy)-2-methyl-propan-1-ol 13-4 (790 mg, yield: 96%) as white gum. MS (ESI) m/z 338.2 [M+18]⁺.

Step 4. A mixture of 13-4 (780 mg, 2.437 mmol) and active MnO₂ (1.7 g, 19.5 mmol) in DCM (100 mL) was stirred for 14 hrs under air at r. t. The reaction mixture was filtered over celite and concentrated. The residue was purified by flash chromatography (25% of EA in PE) to give 2′-fluoro-2-(2-hydroxy-1,1-dimethyl-ethoxy)-5′-methoxy-biphenyl-4-carbaldehyde 13-5 (720 mg, yield: 93%) as pale-yellow gum. ¹H NMR (400 MHz, DMSO-d₆) δ 10.03 (s, 1H), 7.76 (s, 1H), 7.68-7.66 (m, 1H), 7.53 (d, J=8.0 Hz, 1H), 7.21 (t, J=8.8 Hz, 1H), 7.01-6.94 (m, 2H), 4.91 (t, J=4.2 Hz, 1H), 3.76 (s, 3H), 3.28 (d, J=4.2 Hz, 2H), 1.04 (s, 6H).

Step 5. To a mixture of 13-5 (700 mg, 2.2 mmol) and imidazole (224 mg, 3.3 mmol) in THF (15 mL) at 0° C. under N₂ atmosphere was added TBS-C1 (400 mg, 2.64 mmol) in portions. The resulting mixture was stirred for 14 hrs at r. t. Solvent was removed and the residue was purified by flash chromatography (12% of EA in PE) to give 2-[2-(tert-butyl-dimethyl-silanyloxy)-1-methyl-ethoxy]-2′-fluoro-5′-methoxy-biphenyl-4-carbaldehyde 13-6 (550 mg, yield: 58%) as yellow gum. ¹H NMR (400 MHZ, CDCl₃) δ 10.00 (s, 1H), 7.72 (s, 1H), 7.63-7.60 (m, 1H), 7.49-7.46 (m, 1H), 7.05 (t, J=8.8 Hz, 1H), 6.92-6.85 (m, 2H), 3.79 (s, 3H), 3.38 (s, 2H), 1.10 (s, 6H), 0.89 (s, 9H), 0.00 (s, 6H).

Step 6. To a mixture of 13-6 (650 mg, 1.51 mmol) in MeOH (30 mL) at 0° C. under N₂ atmosphere was added NaBH₄ (63 mg, 1.65 mmol) in portions. After addition the resulting mixture was stirred for 30 min at r. t. TLC (PE/EA=5:1) indicated the completion of reaction. MeOH was removed and the residue was diluted with water (30 mL), extracted with EA (30 mL×3), dried and concentrated to give cude {2-[2-(tert-utyl-dimethyl-silanyloxy)-1,1-dimethyl-ethoxy]-2′-fluoro-5′-methoxy-biphenyl-4-yl}-methanol, which was dissolved in DCM (40 mL). pTSA (24 mg, 0.124 mmol) and DHP (209 mg, 2.488 mmol) was added. The resulting mixture was stirred for 3 hrs at r. t. TLC (PE/EA=5:1) indicated the completion of reaction. Solvent was removed and the residue was by flash chromatography (15% of EA in PE) to give tert-butyl-{2-[2′-fluoro-5′-methoxy-4-(tetrahydro-pyran-2-yloxymethyl)-biphenyl-2-yloxy]-2-methyl-propoxy}-dimethyl-silane 13-7 (495 mg, yield: 63.7% over 2 step) as yellow gum. ¹H NMR (400 MHZ, DMSO-d₆) δ 7.26 (d, J=7.6 Hz, 1H), 7.18-7.10 (m, 3H), 6.97-6.92 (m, 1H), 6.88-6.85 (m, 1H), 4.70-4.65 (m, 2H), 4.49-4.44 (m, 1H), 3.83-3.77 (m, 1H), 3.73 (s, 3H), 3.52-3.46 (m, 1H), 1.79-1.63 (m, 2H), 1.56-1.44 (m, 4H), 0.98 (s, 6H), 0.83 (s, 9H), −0.02 (s, 6H).

Step 7. To a mixture of tert-butyl-{2-[2′-fluoro-5′-methoxy-4-(tetrahydro-pyran-2-yloxymethyl)-biphenyl-2-yloxy]-2-methyl-propoxy}-dimethyl-silane 13-7 (495 mg, 0.955 mmol) in THF (5 mL) was added TBAF (1.0 M in THF, 1.91 mmol). The resulting mixture was stirred for 4 hrs at r. t. TLC indicated the completion of reaction. Solvent was removed and the residue was by flash chromatography (30% of EA in PE) to give 2-[2′-fluoro-5′-methoxy-4-(tetrahydro-pyran-2-yloxymethyl)-biphenyl-2-yloxy]-2-methyl-propan-1-ol 13-8 (300 mg, yield: 78%) as white oil. MS (ESI) m/z 422.2 [M+18]⁺.

Step 8. To a mixture of 13-18 (250 mg, 0.619 mmol) in THF (6 mL) at 0° C. under N₂ was added NaH (60%, 50 mg, 1.238 mmol) and stirred for 20 min at 0° C. Then 3-bromopropyne (140 mg, 80% in toluene, 0.928 mmol) was addedvis syringe. The resulting mixture was stirred for 3 h, left the temperature slowly warm to r. t. Quenched by aq. NH₄Cl (10 mL), extracted with EA (20 ml×3), dried and concentrated. The residue was by flash chromatography (20% of EA in PE) to give 2-[2-(1,1-dimethyl-2-prop-2-ynyloxy-ethoxy)-2′-fluoro-5′-methoxy-biphenyl-4-ylmethoxy]-tetrahydro-pyran 13-9 (190 mg, yield: 69.3%) as yellow gum. MS (ESI) m/z 460.2 [M+18]⁺.

Step 9. To a mixture of 13-9 (190 mg, 0.43 mmol) in MeOH (5 mL) was added TsOH·H₂O (204 mg, 1.07 mmol). The resulting mixture was stirred for 3 hrs at r. t. TLC (PE/EA=2:1) indicated the completion of reaction. Solvent was removed and the residue was by flash chromatography (30% of EA in PE) to give [2-(1,1-dimethyl-2-prop-2-ynyloxy-ethoxy)-2′-fluoro-5′-methoxy-biphenyl-4-yl]-methanol 13-10 (100 mg, yield: 65%) as pale-yellow solid. MS (ESI) m/z 376.2 [M+18]⁺.

Step 10. A solution of 13-10 (100 mg, 0.279 mmol), Intermediate 5 (61 mg, 0.279 mmol) and PPh₃ (146 mg, 0.558 mmol) in DCM (4 mL) was degassed and filled with N₂ 3 times and cooled to 0° C. DEAD (97 mg, 0.558 mmol) was then added dropwise via syringe. The resulting mixture was then stirred for 12 hrs under N₂, left the temperature slowly warm to r. t. Solvent was removed and the residue was purified by flash chromatography (25% of EA in PE) to give (R)-3-cyclopropyl-3-{3-[2-(1,1-dimethyl-2-prop-2-ynyloxy-ethoxy)-2′-fluoro-5′-methoxy-biphenyl-4-ylmethoxy]-phenyl}-propionic acid methyl ester 13-11 (110 mg, yield: 70.5%) as white gum. MS (ESI) m/z 578.3 [M+18] +

Step 11. A mixture of 13-11 (110 mg, 0.196 mmol) and LiOH·H₂O (82 mg, 1.96 mmol) in water (4 mL), MeOH (4 mL) and THF (8 mL) was stirred at 50° C. for 14 hrs. TLC indicated the completion of reaction. MeOH was removed and the residue was acidified with aqueous HCl until pH reached 3 and extracted with EtOAc (15 mL×3). The combined organic layers were dried over MgSO₄ and concentrated. The residue was purified by preparative HPLC (TFA method) to give Intermediate 13 (92 mg, yield: 86%) as white solid. MS (ESI) m/z 545.1 [M−H]⁻. ¹H NMR (400 MHZ, DMSO-d₆): 7.32-7.14 (m, 5H), 6.95-6.83 (m, 5H), 5.13 (s, 2H), 4.07 (d, J=2 Hz, 2H), 3.75 (s, 3H), 3.38 (br, 1H), 3.27 (s, 2H), 2.68-2.60 (m, 2H), 2.28-2.23 (m, 1H), 1.02-0.95 (m, 1H), 1.00 (s, 6H), 0.51-0.46 (m, 1H), 0.32-0.20 (m, 2H), 0.12-0.08 (m, 1H). MS (ESI) m/z 545.1 [M−H]⁻.

### Synthesis of Intermediate 14

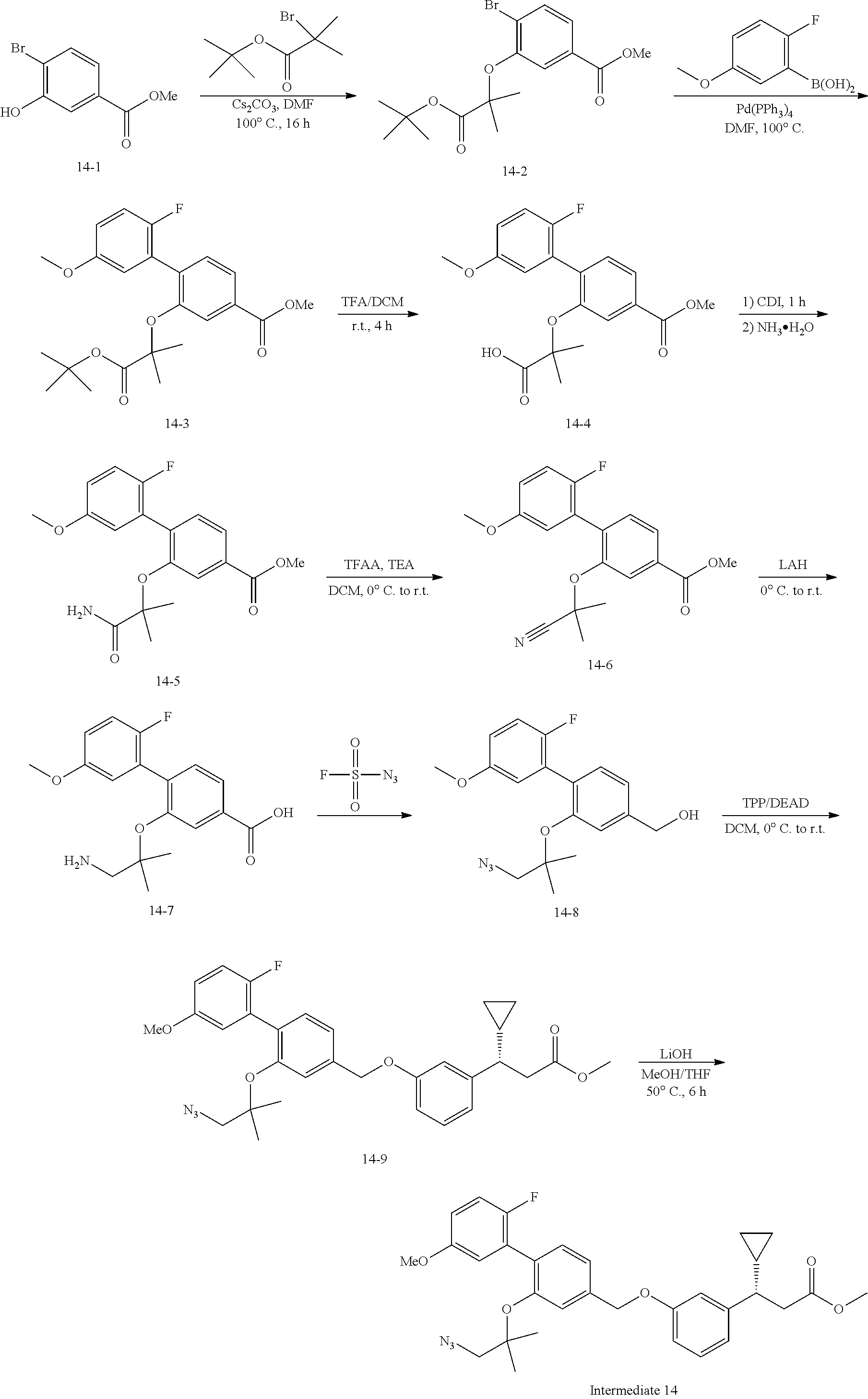

Step 1. A mixture of 4-bromo-3-hydroxy-benzoic acid ethyl ester 14-1 (3.0 g, 12.99 mmol), 2-bromo-2-methyl-propionic acid tert-butyl ester (4.34 g, 19.48 mmol) and Cs₂CO₃ (8.47 g, 25.98 mmol) in DMF (100 mL) was heated at 105° C. for 16 hrs under N₂ atmosphere. The mixture was diluted with water (150 mL), extracted with EA (150 mL×3), dried and concentrated. The residue was purified by flash chromatography (15% EA to 30% EA in PE) to give 4-bromo-3-(1-tert-butoxycarbonyl-1-methyl-ethoxy)-benzoic acid methyl ester 14-2 (2 g, yield: 41%) as white oil. MS (ESI) m/z 390.1 [M+18]⁺.

Step 2. A mixture of 14-2 (2 g, 5.17 mmol), 2-fluoro-5-methoxyphenylboronic acid (1.37 g, 8.06 mmol), (PPh₃)₄ (372 mg, 0.322 mmol) and K₂CO₃ (1.49 g, 10.75 mmol) in DMF (60 mL) was degassed and filled with N₂ 3 times and heated at 100° C. for 16 hrs. The mixture was diluted with water (100 mL), extracted with EA (80 mL×3), dried and concentrated. The residue was purified by flash chromatography (4% to 6% of EA in PE) to give 2-(1-tert-Butoxycarbonyl-1-methyl-ethoxy)-2′-fluoro-5′-methoxy-biphenyl-4-carboxylic acid methyl ester 14-3 (1.0 g, yield: 46.3%) as a yellow gum. MS (ESI) m/z 436.2 [M+18]⁺.

Step 3. To a mixture of 14-3 (1.5 g, 3.58 mmol) in DCM (20 mL) at 0° C. was added TFA (10 mLl) dropwise. After addition the resulting mixture was stirred for 4 hrs at r. t. TLC (PE/EA=5:1) indicated the completion of reaction. The mixture was the diluted with DCM, washed with water and brine, dried and concentrated to give crude 2-(1-carboxy-1-methyl-ethoxy)-2′-fluoro-5′-methoxy-biphenyl-4-carboxylic acid methyl ester 14-4 (1.2 g, yield: 92%) as white solid which was directly used in the next step. MS (ESI) m/z 361.1 [M−H]⁻.

Step 4. To a mixture of 14-4 (1.2 g, 3.315 mmol) in dry THF (40 mL) at 0° C. under N₂ was added CDI (805 mg, 4.97 mmol) in portions. The resulting mixture was stirred for 1 h, allowing the temperature slowly warm to r. t. NH₃.H2O (0.74 mL, 13.26 mmol) was added to the reaction mixture and stirred for additional 3 hrs at r. t. Solvent was removed and the residue was purified by flash chromatography (10% to 50% of EA in PE) to give 2-(1-carbamoyl-1-methyl-ethoxy)-2′-fluoro-5′-methoxy-biphenyl-4-carboxylic acid methyl ester 14-5 (900 mg, yield: 75.6%) as white solid. MS (ESI) m/z 362.1 [M+H]⁺.

Step 5. To a mixture of 14-5 (850 mg, 2.354 mmol) and TEA (1.2 g, 11.77 mmol) in DCM (80 mL) at 0° C. under N₂ atmosphere was added TFAA (1.98 g, 9.418 mmol) dropwise. The resulting mixture was stirred for 14 hrs at r. t. Solvent was removed and the residue was purified by flash chromatography (18% of EA in PE) to give 2-(cyano-dimethyl-methoxy)-2′-fluoro-5′-methoxy-biphenyl-4-carboxylic acid methyl ester 14-6 (700 mg, yield: 86%) as yellow solid. MS (ESI) m/z 361.2 [M+18]⁺. ¹H NMR (400 MHZ, DMSO-d₆) δ 7.98 (s, 1H), 7.86-7.83 (m, 1H), 7.57 (d, J=8.0 Hz, 1H), 7.22 (t, J=9.2 Hz, 1H), 7.03-6.99 (m, 1H), 6.93-6.92 (m, 1H), 3.90 (s, 3H), 3.77 (s, 3H), 1.56 (s, 6H).

Step 6. To a mixture of 14-6 (400 mg, 1.16 mmol) in THF (30 mL) at 0° C. under N2 atmosphere was added LAH (350 mg, 9.2 mmol) in portions. The resulting mixture was stirred for 12 hrs at r. t. TLC (PE/EA=2:1) indicated the completion of reaction. The reaction mixture was cooled to 0° C. and quenched with water (0.35 mL), aq. NaOH (15%, 0.35 mL) and water (1.05 mL) successively. The mixture was filtered and the filter cake was washed with EA (20 mL×3). The combined organic phase was dried and concentrated to give crude [2-(2-amino-1,1-dimethyl-ethoxy)-2′-fluoro-5′-methoxy-biphenyl-4-yl]-methanol 14-7 (380 mg) as yellow gum which was directly used in the next step. MS (ESI) m/z 320.2 [M+H]⁺.

Step 7. To a mixture of 14-7 (380 mg, 1.19 mmol) in DMF (10 mL) was added fluorosulfuryl azide (˜0.5 M in MTBE, 1.58 mmol) and KHCO₃ (3.0 M, 1.32 mL, 3.948 mmol). The resulting mixture was stirred for 12 hrs at r. t. The reaction mixture was diluted with water (40 mL), extracted with EA (30 mL×3), dried and concentrated. The residue was by flash chromatography (20% of EA in PE) to give [2-(2-azido-1,1-dimethyl-ethoxy)-2′-fluoro-5′-methoxy-biphenyl-4-yl]-methanol 14-8 (280 mg, yield: 68%) as yellow gum. MS (ESI) m/z 363.2 [M+18]⁺.

Step 8. A solution of 14-8 (98 mg, 0.284 mmol), (R)-3-cyclopropyl-3-(3-hydroxy-phenyl)-propionic acid methyl ester (62 mg, 0.284 mmol) and PPh₃ (149 mg, 0.568 mmol) in DCM (4 mL) was degassed and filled with N₂ 3 times and cooled to 0° C. DEAD (98 mg, 0.568 mmol) was then added dropwise via syringe. The resulting mixture was then stirred for 12 hrs under N₂, left the temperature slowly warm to r. t. Solvent was removed and the residue was purified by flash chromatography (8% of EA in PE) to give (R)-3-{3-[2-(2-Azido-1,1-dimethyl-ethoxy)-2′-fluoro-5′-methoxy-biphenyl-4-ylmethoxy]-phenyl}-3-cyclopropyl-propionic acid methyl ester 14-9 (55 mg, yield: 35%) as colorless oil. MS (ESI) m/z 565.3 [M+18]⁺.

Step 9. A mixture of 14-9 (165 mg, 0.302 mmol) and LiOH·H₂O (126 mg, 3.02 mmol) in water (5 mL), MeOH (5 mL) and THF (10 mL) was stirred at 50° C. for 5 hrs. TLC indicated the completion of reaction. MeOH was removed and the residue was acidified with aqueous HCl until pH reached 3 and extracted with EtOAc (15 mL×3). The combined organic layer was dried and concentrated. The residue was purified by preparative HPLC (TFA method) to give Intermediate 14 (130 mg, yield: 81%) as yellow solid. MS (ESI) m/z 532.1 [M−H]⁻. ¹H NMR (400 MHZ, DMSO-d₆): 12 (br, 1H), 7.33-7.14 (m, 5H), 6.96-6.83 (m, 5H), 5.15 (s, 2H), 3.75 (s, 3H), 3.23 (s, 2H), 2.70-2.60 (m, 2H), 2.27-2.22 (m, 1H), 1.01 (s, 6H), 1.01-0.97 (m, 1H), 0.52-0.47 (m, 1H), 0.30-0.21 (m, 2H), 0.12-0.08 (m, 1H). MS (ESI) m/z 532.1 [M−H]⁻.

### Synthesis of Intermediate 15

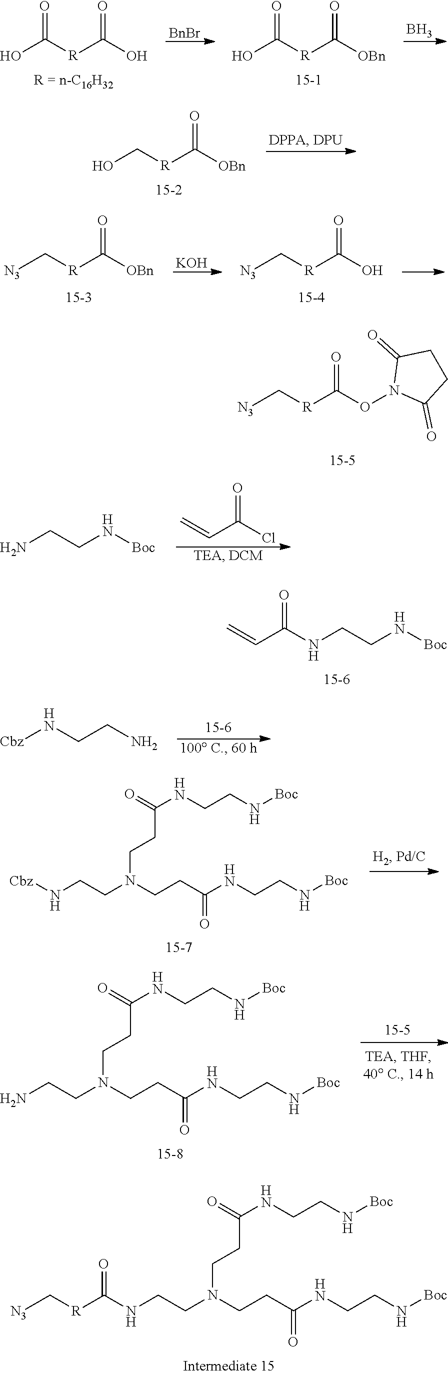

Step 1. To a solution of octadecanedioic acid (10 g, 0.032 mol) in DMF (150 mL) was added BnBr (5.5 g, 0.032 mol) and K₂CO₃ (6.6 g, 0.048 mol) at room temperature. The mixture was stirred at 8° C. overnight. The pH was adjusted to 2 with the addition of 0.5 N HCl. The resulting solution was extracted with EA (200 mL×3). The organic layer was washed with brine (200 mL×3), dried over Na₂SO₄ and filtered. The filtrate was concentrated, and the residue was purified by silica gel column chromatography (PE/EA=3/1, v/v) to afford 18-(benzyloxy)-18-oxooctadecanoic acid, 15-1, as a white solid. ¹H NMR (400 MHZ, DMSO-d₆): δ=11.94 (s, 1H), 7.38-7.31 (m, 5H), 5.08 (s, 2H), 2.34 (t, 2H), 2.18 (t, 2H), 1.54-1.46 (m, 4H), 1.23 (s, 24H).

Step 2. To a solution of 15-1 (6.0 g, 0.015 mol) in THF (50 mL) was added BH₃/THF (1.0M, 45 mL, 0.045 mmol) at 0° C. The resulting reaction mixture was stirred overnight. Then the reaction was quenched with MeOH (50 mL) and concentrated in vacuo. The residue was purified by silica gel column chromatography (PE/EA=5/1, v/v) to afford benzyl 18-hydroxy octadecanoate, 15-2, as a white solid. ¹H NMR (400 MHZ, DMSO-d₆): δ =7.37-7.32 (m, 5H), 5.08 (s, 2H), 4.30 (t, 1H), 3.39-3.34 (m, 2H), 2.34 (t, 2H), 1.54-1.51 (m, 2H), 1.41-1.37 (m, 2H), 1.23 (s, 26H).

Step 3. To a solution of 15-2 (7.4 g, 0.019 mol) in DMF (100 mL) was added DPPA (7.8 g, 0.028 mol) and DBU (4.3 g, 0.028 mol) at room temperature. The mixture was stirred at 90° C. overnight. The residue was poured into water (100 mL), extracted with EA (200 mL×2). The organic layer was washed with brine (100 mL×2), dried over Na₂SO₄ and filtered. The filtrate was concentrated, and the residue was purified by silica gel column chromatography (PE) to afford benzyl 18-azidooctadecanoate, 15-3, as a white solid. ¹H NMR (400 MHZ, CDCl₃): δ=7.37-7.32 (m, 5H), 5.11 (s, 2H), 3.25 (t, 2H), 2.35 (t, 2H), 1.66-1.56 (m, 4H), 1.38-1.25 (m, 26H).

Step 4. To a solution of 15-3 (1.0 g, 2.41 mmol) in MeOH/H2O (10 mL/5 mL) was added KOH (675 mg, 12.05 mmol) at 0° C. The mixture was stirred at 0° C. for 2 h. Then the reaction was concentrated in vacuo, acidified to pH=1 by 1 N HCl and extracted by EA. The organic layer was combined, dried over Na₂SO₄ and filtered. The filtrate was concentrated afford 18-azidooctadecanoic acid, 15-4 (550 mg, yield: 70.2%) as a white solid. ¹H NMR (400 MHZ, DMSO-d₆): δ=11.99 (s, 1H), 3.30 (t, 2H), 2.18 (t, 2H), 1.54-1.46 (m, 4H), 1.25-1.20 (m, 26H).

Step 5. To a mixture of 18-azido-octadecanoic acid, 15-4, (1 g, 3.07 mmol) and 1-hydroxy-pyrrolidine-2,5-dione (372 mg, 3.23 mmol) in DCM (50 mL) was added EDCI (650 mg, 3.383 mmol) in portions at r. t. (15° C.). After addition, the resulting mixture was stirred for 14 hr at r. t. The reaction mixture was concentrated and the residue was purified by silica gel column chromatography (50% to 100% DCM in PE) to give 18-azido-octadecanoic acid 2,5-dioxo-pyrrolidin-1-yl ester, 15-5, as a white solid. ¹H NMR (400 MHZ, CDCl₃): δ=3.25 (t, 2H), 2.83 (brs, 4H), 2.60 (t, 2H), 1.76-1.72 (m, 2H), 1.62-1.56 (m, 2H), 1.41-1.30 (m, 2H), 1.25-1.3 (m, 24H).

Step 6. To a mixture of (2-amino-ethyl)-carbamic acid tert-butyl ester (8 g, 0.005 mol) and TEA (15.3 g, 0.15 mol) in DCM (120 mL) at 0° C. was added acryloyl chloride (6.787 g, 0.075 mol, caution: tears!) dropwise during 5 min. After addition, the resulting mixture was stirred for 16 hr, allowing the temperature slowly warm to r. t. The reaction mixture was quenched with aq. NaHCO₃ and separated, extracted with DCM (50 mL×2). The combined organic phase was dried and concentrated. The residue was purified by flash chromatography (60% EA in PE) to give (2-acryloylamino-ethyl)-carbamic acid tert-butyl ester 15-6 (5.5 g, yield: 51%) as pale-yellow solid. MS (ESI) m/z 158.9 [M-55]⁺. ¹H NMR (400 MHZ, CDCl₃): δ=6.46 (br, 1H), 6.28-6.24 (d, J=16.8 Hz, 1H), 6.13-6.07 (dd, J=16.8 Hz, 10.4 Hz, 1H), 5.65-5.62 (d, J=10.4 Hz, 1H), 4.98 (br, 1H), 3.46-3.42 (m, 2H), 3.33-3.29 (m, 2H), 1.43 (s, 9H).

Step 7. A pressure tube charged with (2-amino-ethyl)-carbamic acid benzyl ester (500 mg, 2.577 mmol) and (2-acryloylamino-ethyl)-carbamic acid tert-butyl ester 15-6 (2.76 g, 12.886 mmol) in sat. aq. HBO₃ (5 mL) was sealed and heated at 100° C. for 2 days. The reaction mixture was diluted with water (10 mL), extracted with DCM (30 mL×4) and concentrated. The residue was purified by flash chromatography (10% MeOH in DCM, @214 nm) to give (2-{bis-[2-(2-tert-butoxycarbonylamino-ethylcarbamoyl)-ethyl]-amino}-ethyl)-carbamic acid benzyl ester 15-7 (550 mg, yield: 35%) as white solid. MS (ESI) m/z 623.1 [M+H]⁺.

Step 8. A flask charged with 15-7 (550 mg, 0.884 mmol) and Pd/C (˜270 mg, 50% w.t.) in MeOH (50 mL) was degassed and filled with hydrogen using a balloon. The resulting mixture was then hydrogenated at 25° C. for 16 hrs. The reaction was filter over celite and concentrated. The residue was purified by flash chromatography (28% MeOH in DCM, 0.5% NH₃.H2O) to give [2-(3-{(2-amino-ethyl)-[2-(2-tert-butoxycarbonylamino-ethylcarbamoyl)-ethyl]-amino}-propionylamino)-ethyl]-carbamic acid tert-butyl ester, 15-8 (330 mg, yield: 76) as pale-yellow solid. MS (ESI) m/z 489.5 [M+H]⁺. ¹H NMR (400 MHZ, DMSO-d₆): δ=7.99 (br, NH, 2H), 6.86 (br, NH, 2H), 3.11-3.07 (m, 4H), 3.03-2.99 (m, 4H), 2.66-2.62 (m, 6H), 2.45-2.40 (m, 2H), 2.24-2.20 (m, 4H), 1.42 (s, 18H).

Step 9. To a mixture of 15-8 (250 mg, 0.506 mmol) and TEA (78 mg, 0.759 mmol) in THF (15 mL) was added 15-5 (235 mg, 0.556 mmol). After addition, the resulting mixture was stirred at 40° C. for 16 hrs. Solvent was removed and the residue (in MeOH) was purified by preparative HPLC (NH₄HCO₃ method) to give [2-(3-{[2-(18-Azido-octadecanoylamino)-ethyl]-[2-(2-tert-butoxycarbonylamino-ethylcarbamoyl)-ethyl]-amino}-propionylamino)-ethyl]-carbamic acid tert-butyl ester, Intermediate 15, as pale-yellow solid.

MS (ESI) m/z 796.8 [M+H]⁺. ¹H NMR (400 MHZ, DMSO-d₆): δ=7.92 (t, N|H, 2H), 7.61 (t, NH, 1H), 6.80 (t, NH, 2H), 3.30 (t, 2H), 3.11-3.05 (m, 6H), 3.04-2.97 (m, 4H), 2.69-2.64 (m, 4H), 2.46-2.41 (m, 2H), 2.22-2.17 (m, 4H), 2.06 (t, 2H), 1.60-1.49 (m, 4H), 1.48 (s, 18H), 1.46-1.25 (m, 26H).

### Synthesis of Intermediate 16

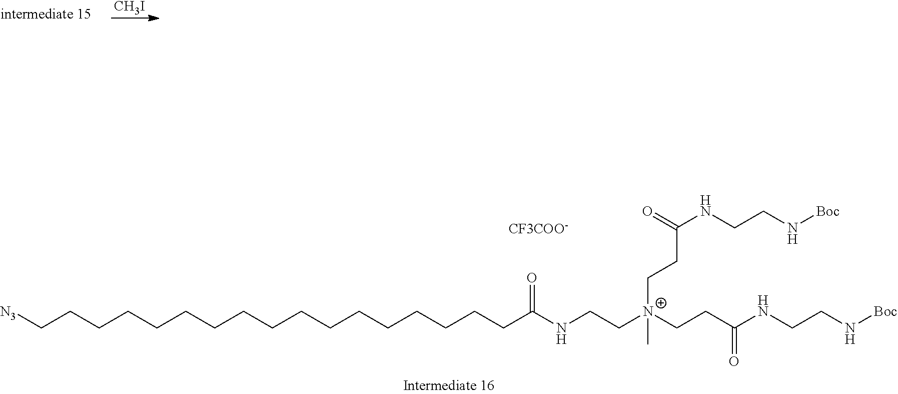 [SMILES: "CH3I" ; CC(C)(C)OC(=O)NCCNC(=O)CC[N+](C)(CCNC(=O)CCCCCCCCCCCCCCCCCN=[N+]=[N-])CCC(=O)NCCNC(=O)OC(C)(C)C ; "CF3COO-"]

A pressure tube (20 mL) charged with Intermediate 15 (500 mg, 0.63 mmol) and Mel (1 mL, 16 mmol) in MeCN(6 mL) was sealed and heated at 60° C. for 14 hrs. Solvent was removed and the residue (in MeOH) was purified by preparative HPLC (C8 column, TFA method) to give [2-(18-azido-octadecanoylamino)-ethyl]-bis-[2-(2-tert-butoxycarbonylamino-ethyl carbamoyl)ethyl]-methyl-ammonium, Intermediate 16 (320 mg, yield: 62%) as colorless gum. MS (ESI) m/z 810.6 [M+]⁺. ¹H NMR (400 MHZ, DMSO-d₆): δ =8.18 (t, NH, 2H), 8.11 (t, NH, 1H), 6.85 (t, NH, 2H), 3.60-3.55 (m, 4H), 3.47-3.40 (m, 2H), 3.35-3.28 (m, 4H), 3.12-3.08 (m, 4H), 3.05-2.98 (m, 7H), 2.66-2.61 (m, 4H), 2.10 (t, 2H), 1.56-1.51 (m, 4H), 1.40 (s, 18H), 1.30-1.20 (m, 26H).

### Synthesis of Intermediate 17

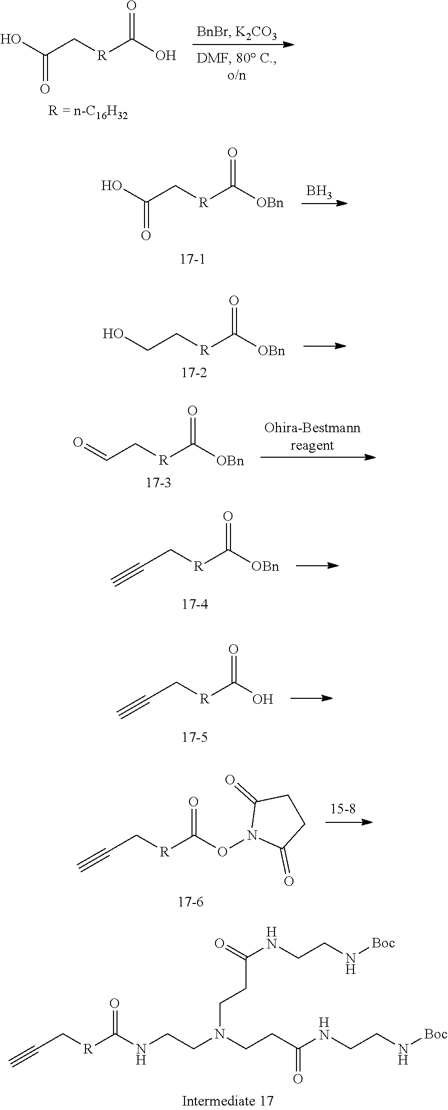

Step 1. To a solution of nonadecanedioic acid (23.0 g, 70.2 mmol) in DMF (400 mL) at room temperature was added K₂CO₃ (19.4 g, 140.4 mmol), BnBr (12.0 g, 70.2 mmol). The reaction mixture was stirred at 80° C. overnight under N₂. The reaction mixture was diluted with solvent and poured into water. The mixture was acidified to pH 3-4 with aqueous HCl and the mixture was extracted with EA (20 mL×4). The combined organic layers were dried over MgSO₄ and concentrated. The crude product was chromatographed on silica gel (Petroleum ether/EtOAc 10:1→5:1→0:1→MeOH/EtOAc 1:10) to give compound 17-1 (15.8 g, 54%) as a white solid. MS (ESI) m/z 417.2 [M−H]⁻. ¹H NMR (400 MHZ, CDCl₃) δ 7.42-7.35 (m, 5H), 5.12 (s, 2H), 2.35 (m, 4H), 1.73-1.62 (m, 4H), 1.31-1.17 (m, 26H).

Step 2. To a solution of compound 17-1 (2.22 g, 5.3 mmol) in THF (25 mL) cooled to 0° C. was added dropwise BH₃ (5.3 mL, 10.6 mmol) under N₂. The reaction mixture was stirred at room temperature for 2 hours. The reaction mixture was quenched by the addition of MeOH at 0° C. The reaction mixture was concentrated under reduced pressure. The crude product was chromatographed on silica gel (Petroleum ether/EtOAc 10:1→7:1→* 4:1) to give compound 17-2 (1515 mg, 71%) as a white solid. MS (ESI) m/z 405.2 [M+H]⁺. ¹H NMR (400 MHZ, CDCl₃) δ 7.36-7.32 (m, 5H), 5.11 (s, 2H), 4.31 (t, 1H), 3.35 (dt, 2H), 2.34 (t, 2H), 1.53 (m, 2H), 1.39 (m, 2H), 1.55-1.25 (m, 26H).

Step 3. To a mixture of compound 17-2 (15.2 g, 3.7 mmol) in dry DCM (30 mL) was added PCC (16.0 g, 7.4 mmol) under N₂. The reaction mixture was stirred at room temperature overnight under N₂. The reaction mixture was concentrated under reduced pressure. The crude product was chromatographed on silica gel (Petroleum ether/EtOAc 1:0 →10:1) to give compound 17-3 (587 mg, 39%) as a pale-yellow solid. MS (ESI) m/z 403.2 [M+H]⁺. ¹H NMR (400 MHz, CDCl₃) δ 9.76 (t, J=2.4 Hz, 1H), 7.36-7.31 (m, 5H), 5.11 (s, 2H), 2.41 (dt, J=9.6 Hz, 2.4 Hz, 2H), 2.35 (t, J=10 Hz, 2H), 1.64-1.55 (m, 4H), 1.32-1.24 (m, 26H).

Step 4. To a mixture of compound 17-3 (587 mg, 1.5 mmol) in MeOH (10 mL) cooled to 0° C. was added Ohira-Bestmann reagent (437 mg, 2.25 mmol), K₂CO₃ (311 mg, 2.25 mmol) under N₂. The reaction mixture was stirred at room temperature overnight under N2. The reaction mixture was concentrated under reduced pressure. The crude product was chromatographed on silica gel (Petroleum ether/EtOAc 1:0-20:1) to give compound 17-4 (373 mg, 79%) as a pale-yellow solid. ¹H NMR (400 MHZ, CDCl₃) δ 3.66 (s, 3H), 2.30 (t, J=10.2 Hz, 2H), 2.18 (dt, J=9.4, 3.6 Hz, 2H), 1.93 (t, J=3.4 Hz, 1H), 1.64-1.59 (m, 2H), 1.52-1.47 (m, 2H), 1.40-1.36 (m, 2H), 1.36-1.25 (m, 24H).

Step 5. To a solution of compound 17-4 (373 mg, 1.2 mmol) in MeOH (10 mL), THF (10 mL), H₂O (10 mL) at room temperature was added KOH (202 mg, 3.6 mmol). The reaction mixture was stirred at 50° C. overnight. The reaction mixture was concentrated under reduced pressure. The residue was diluted with solvent and poured into water. The aqueous phase was acidified with aqueous HCl and extracted with EtOAc (15 mL×4). The combined organic layers were dried over MgSO₄ and concentrated. The crude product was chromatographed on silica gel (Petroleum ether/EtOAc 1:0→10:1) to give 17-5 (368 mg, 100%) as a white solid. MS (ESI) m/z 307.2 [M−H]⁻. ¹H NMR (400 MHZ, CDCl₃) δ 2.35 (t, 2H), 2.20-2.15 (m, 2H), 1.93 (t, J=2.6 Hz, 1H), 1.66-1.50 (m, 4H), 1.40-1.25 (m, 26H).

Step 6. To a solution of 17-5 (2.0 g, 6.5 mmol) and 1-hydroxy-pyrrolidine-2,5-dione (1.5 g, 12.9 mmol) in DCM (40 mL) was added EDCI (2.5 g, 12.945 mmol) in portions at room temperature. The reaction mixture was stirred at room temperature for 16 hours. The reaction mixture was quenched with water (40 mL) and extracted with DCM (30 mL×3). The combined organic layers were washed with water (40 mL×2) and brine (40 mL), dried over Na₂SO₄ and concentrated. The residue was purified by chromatography on silica gel (EA/PE=0-10%) to give 2,5-dioxopyrrolidin-1-yl icos-19-ynoate, 17-6 (1.4 g, yield: 79.1%) as a white solid. ¹H NMR (400 MHZ, CDCl₃): δ=2.83 (brs, 4H), 2.60 (t, 2H), 2.18 (td, J=7 Hz, 2.5 Hz, 2H), 1.93 (t, J=2.5 Hz, 1H), 1.77-1.72 (m, 2H), 1.45-1.50 (m, 2H), 1.35-1.40 (m, 4H), 1.25-1.30 (22H).

Step 7. A mixture of 17-6 (280 mg, 0.691 mmol) and 15-8 (340 mg, 0.69 mmol) and TEA (106 mg, 1.036 mmol) in THF (10 mL) was stirred at 40° C. for 16 h. Solvent was removed and the residue (in MeOH) was purified by preparative HPLC (NH₄HCO₃ method) to give (2-{3-[[2-(2-tert-butoxycarbonylamino-ethylcarbamoyl)-ethyl]-(2-icos-19-ynoylamino-ethyl)-amino]-propionyl amino}-ethyl)-carbamic acid tert-butyl ester,

Intermediate 17 (280 mg, yield: 52%) as pale-yellow solid. MS (ESI) m/z 779.6 [M+H]⁺. ¹H NMR (400 MHZ, DMSO-d₆): δ=7.88 (t, NH, 2H), 7.56 (t, NH, 1H), 6.75 (t, NH, 2H), 3.10-3.03 (m, 6H), 2.99-2.92 (m, 4H), 2.72 (t, J=2.8 Hz, 1H), 2.67-2.61 (m, 4H), 2.43-2.39 (m, 2H), 2.19-2.11 (m, 6H), 2.03 (t, 2H), 1.49-1.30 (m, 4H), 1.37 (s, 18H), 1.30-1.19 (m, 26H).

### Synthesis of Intermediate 18

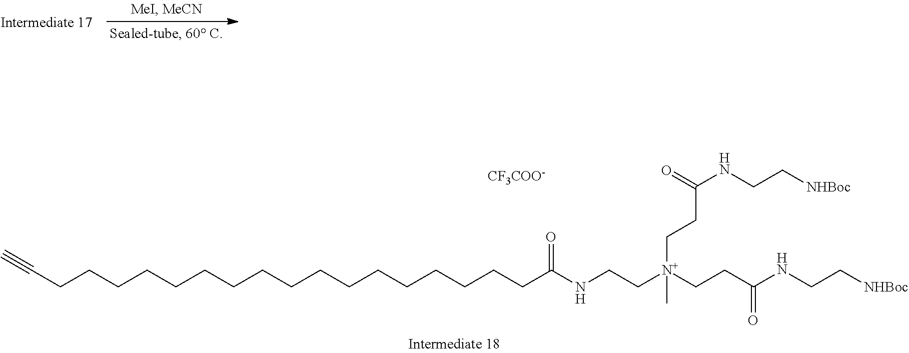 [SMILES: C#CCCCCCCCCCCCCCCCCCC(=O)NCC[N+](C)(CCC(=O)NCCNC(=O)OC(C)(C)C)CCC(=O)NCCNC(=O)OC(C)(C)C ; "CF3COO-"]

A pressure tube (8 mL) charged with Intermediate 17 (200 mg, 0.256 mmol) and MeI (0.5 mL, 8 mmol) in MeCN(2 mL) was sealed and heated at 60° C. for 14 h. Solvent was removed and the residue (in MeOH) was purified by prep-HPLC (C8 column, TFA method) to give bis-[2-(2-tert-butoxycarbonylamino-ethylcarbamoyl)-ethyl]-(2-icos-19-ynoyl amino-ethyl)-methyl-ammonium, Intermediate 18 (120 mg, yield: 59%) as colorless gum. MS (ESI) m/z 793.6 [M+]⁺. ¹H NMR (400 MHZ, DMSO-d₆): δ=8.15 (t, NH, 2H), 8.08 (t, NH, 1H), 6.83 (t, NH, 2H), 3.57-3.53 (m, 4H), 3.40-3.25 (m, 4H), 3.12-3.06 (m, 4H), 3.05-2.96 (m, 7H), 2.73 (t, 1H), 2.64-2.59 (m 4H), 2.16-2.06 (m, 4H), 1.52-1.31 (m, 4H), 1.37 (s, 18H), 1.30-1.17 (m, 26H).

The following intermediates were synthesized in the same procedures as for Intermediate 15, 16, 17, 18, using reagents that differ in the alkyl chain lengths.

### Synthesis of Intermediate 19

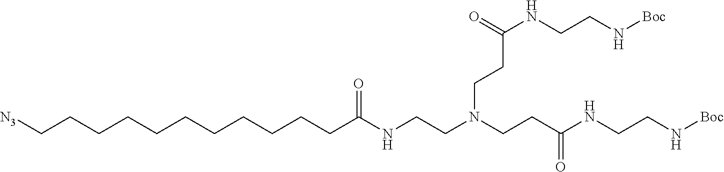 [SMILES: CC(C)(C)OC(=O)NCCNC(=O)CCN(CCNC(=O)CCCCCCCCCCCN=[N+]=[N-])CCC(=O)NCCNC(=O)OC(C)(C)C]

MS (ESI) m/z 712.4 [M+H]⁺. ¹H NMR (400 MHZ, DMSO-d₆): δ 7.89 (t, NH, 2H), 7.58 (t, NH, 1H), 6.76 (t, NH, 2H), 3.37-3.29 (t, 2H), 3.10-3.03 (m, 6H), 2.99-2.94 (m, 4H), 2.66-2.59 (m, 4H), 2.43-2.2.38 (m, 2H), 2.19-2.14 (m, 4H), 2.05-2.01 (t, 2H), 1.54-1.45 (m, 4H), 1.37 (s, 18H, 1.30-1.17 (m, 14H).

### Synthesis of Intermediate 20

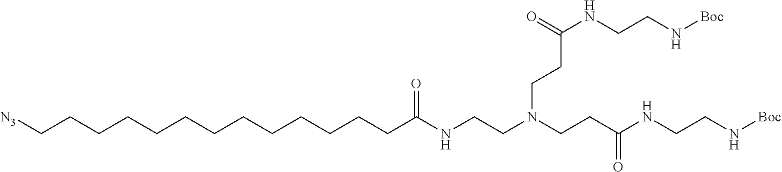 [SMILES: CC(C)(C)OC(=O)NCCNC(=O)CCN(CCNC(=O)CCCCCCCCCCCCCN=[N+]=[N-])CCC(=O)NCCNC(=O)OC(C)(C)C]

MS (ESI) m/z 740.5 [M+H]⁺. ¹H NMR (400 MHZ, DMSO-d₆): δ 7.88 (t, NH, 2H), 7.57 (t, NH, 1H), 6.76 (t, NH, 2H), 3.37-3.29 (t, 2H), 3.10-3.03 (m, 6H), 2.99-2.94 (m, 4H), 2.66-2.59 (m, 4H), 2.43-2.2.38 (m, 2H), 2.19-2.14 (m, 4H), 2.05-2.01 (t, 2H), 1.54-1.45 (m, 4H), 1.37 (s, 18H, 1.30-1.17 (m, 18H).

### Synthesis of Intermediate 21

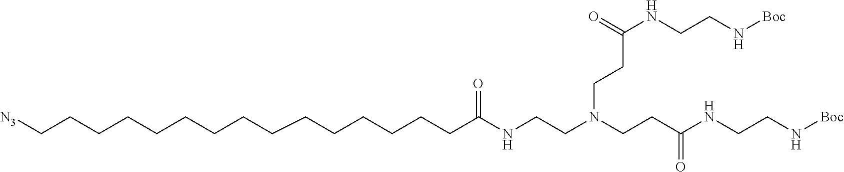 [SMILES: CC(C)(C)OC(=O)NCCNC(=O)CCN(CCNC(=O)CCCCCCCCCCCCCCCN=[N+]=[N-])CCC(=O)NCCNC(=O)OC(C)(C)C]

MS (ESI) m/z 768.6 [M+]⁺. ¹H NMR (400 MHZ, DMSO-d₆): δ 7.88 (t, NH, 2H), 7.57 (t, NH, 1H), 6.75 (t, NH, 2H), 3.30 (t, 2H), 3.10-3.03 (m, 6H), 2.99-2.94 (m, 4H), 2.66-2.59 (m, 4H), 2.43-2.39 (m, 2H), 2.19-2.15 (m, 4H), 2.05-2.00 (t, 2H), 1.55-1.34 (m, 4H), 1.37 (s, 18H), 1.32-1.15 (m, 22H).

### Synthesis of Intermediate 22

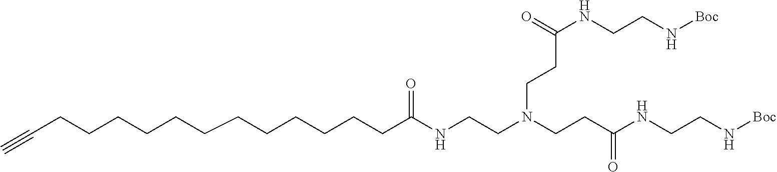 [SMILES: C#CCCCCCCCCCCCCC(=O)NCCN(CCC(=O)NCCNC(=O)OC(C)(C)C)CCC(=O)NCCNC(=O)OC(C)(C)C]

MS (ESI) m/z 709.4 [M+]⁺. ¹H NMR (400 MHZ, CDCl₃): δ 7.25 (br, NH, 2H), 6.90 (br, NH, 1H), 5.47 (br, NH, 2H), 3.34-3.30 (m, 6H), 3.25-3.24 (m, 4H), 2.70-2.69 (m, 4H), 2.53-2.50 (m, 2H), 2.35-2.32 (m, 4H), 2.19-2.16 (m 4H), 1.61-1.59 (m, 2H), 1.55-1.48 (m, 2H), 1.43 (s, 18H), 1.25 (br, 16H).

### Synthesis of Intermediate 23

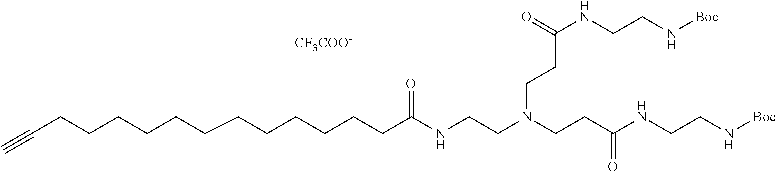 [SMILES: C#CCCCCCCCCCCCCC(=O)NCCN(CCC(=O)NCCNC(=O)OC(C)(C)C)CCC(=O)NCCNC(=O)OC(C)(C)C ; "CF3COO-"]

MS (ESI) m/z 723.5 [M+]⁺. ¹H NMR (400 MHZ, DMSO-d₆): δ 8.14 (t, NH, 2H), 8.07 (t, NH, 1H), 6.82 (t, NH, 2H), 3.57-3.52 (m, 4H), 3.45-3.41 (m, 2H), 3.30-3.26 (m, 2H), 3.10-3.06 (m, 4H), 3.00 (s, 3H), 3.00-2.96 (m, 4H), 2.73-2.72 (t, 1H), 2.64-2.59 (m, 4H), 2.16-2.07 (m, 4H), 1.50-1.30 (m, 4H), 1.38 (s, 18H), 1.30-1.20 (m, 16H).

### Synthesis of Intermediate 24

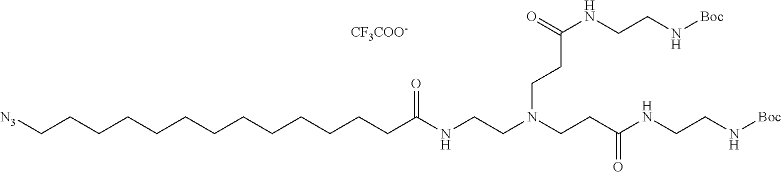 [SMILES: CC(C)(C)OC(=O)NCCNC(=O)CCN(CCNC(=O)CCCCCCCCCCCCCN=[N+]=[N-])CCC(=O)NCCNC(=O)OC(C)(C)C ; "CF3COO-"]

MS (ESI) m/z 754.5 [M+]⁺. ¹H NMR (400 MHz, DMSO-d₆): δ 8.14 (t, NH, 2H), 8.06 (t, t, NH, 1H), 6.82 (t, NH, 2H), 3.57-3.52 (m, 4H), 3.44-3.41 (m, 2H), 3.33-3.25 (m, 4H), 3.11-3.06 (m, 4H), 3.02-2.96 (m, 7H), 2.63-2.59 (m, 4H), 2.08 (t, 2H), 1.53-1.39 (m, 4H), 1.38 (s, 18H), 1.35-1.20 (m, 18H).

### Synthesis of Intermediate 25

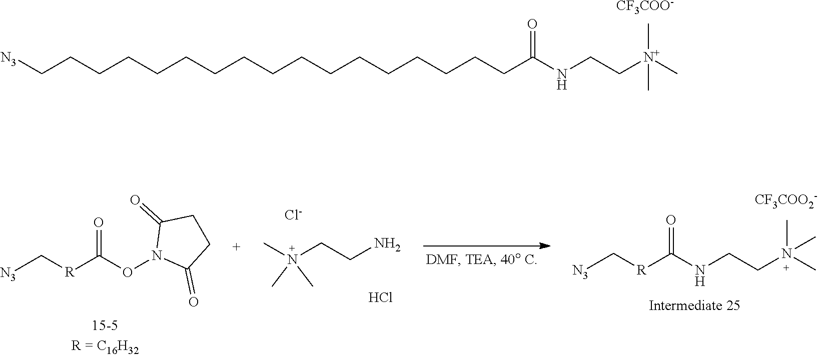

A mixture of 2,5-dioxopyrrolidin-1-yl 18-azidooctadecanoate (15-5, 300 mg, 0.71 mmol), 2-amino-N,N,N-trimethylethan −1-aminium chloride hydrogen chloride (100 mg, 0.72 mmol) and TEA (360 mg, 3.55 mmol) in DMF (10 mL) was heated at 40° C. for 14 hrs. The mixture became clear. Solvent was removed in vacuo and the residue was diluted with MeOH (3 mL) and DMF (1 mL), filtered, and the filtrate was purified by prep-HPLC to give 2-(18-azido-octadecanamido)-N,N,N-trimethylethan −1-aminium trifluoroacetate (Intermediate 25, 240 mg, yield: 65.7%) as a white solid. MS (ESI) m/z 410.4 [M+H]⁺. ¹H NMR (400 MHZ, DMSO-d₆): δ=8.11 (s, 1H), 3.41-3.28 (m, 6H), 3.08 (s, 9H), 2.11-2.06 (t, 2H), 1.52-1.48 (m, 4H), 1.37-1.17 (m, 26H).

### Synthesis of Intermediate 26

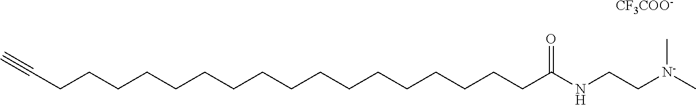

Intermediate 26 was prepared from compound 17-6 and 2-amino-N,N,N-trimethylethan −1-aminium chloride hydrogen chloride in the same way as for Intermediate 25. MS (ESI) m/z 393.4 [M+]⁺. ¹H NMR (400 MHZ, DMSO-d₆): δ 8.12-8.09 (t, NH, 1H), 3.47-3.42 (m, 2H), 3.35-3.31 (t, 2H), 3.08 (s, 9H), 2.74-2.72 (t, 1H), 2.15-2.07 (m, 4H), 1.51-1.37 (m, 4H), 1.35-1.17 (m, 26H).

### Synthesis of Intermediate 27

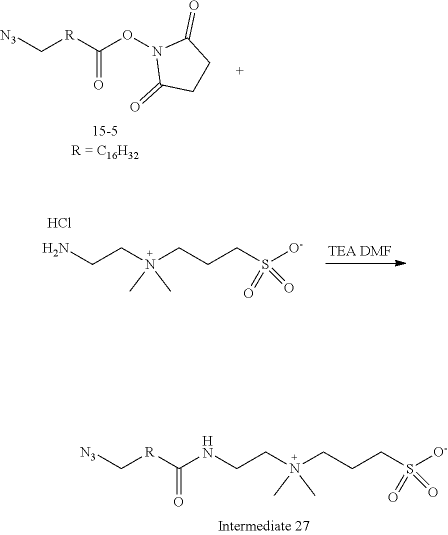

A mixture of compound 15-5, (149 mg, 0.35 mmol), 3-((2-aminoethyl)dimethylammonio) propane-1-sulfonate hydrochloride (100 mg, 0.35 mmol) and TEA (179 mg, 1.77 mmol) in DMF (5 mL) was heated at 50° C. for 14 hrs. The white suspension was concentrated in vacuo and the residue was diluted with MeOH (3 mL) and DMF (0.5 mL), filtered, and the filtrate was purified by prep-HPLC (TFA method, collected by MS trigger) to give 3-((2-(18-azido-octadecanamido)ethyl) dimethylammonio) propane-1-sulfonate, Intermediate 27, 60 mg, yield: 32.8% as a white solid. MS (ESI) m/z 518.4 [M+]⁺. ¹H NMR (400 MHZ, CD₃OD) δ 3.65-3.62 (m, 2H), 3.58-3.54 (m, 2H), 3.44-3.40 (t, 2H), 3.30-3.24 (t, 2H), 3.15 (s, 9H), 2.87-2.82 (t, 2H), 2.24-2.18 (m, 4H), 1.65-1.56 (m, 4H), 1.40-1.25 (m, 26H).

### Synthesis of Intermediate 28

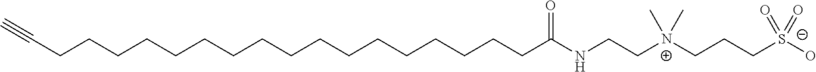 [SMILES: C#CCCCCCCCCCCCCCCCCCC(=O)NCC[N+](C)(C)CCCS(=O)(=O)[O-]]

Intermediate 28 was prepared from compound 17-6 and 3-((2-aminoethyl)dimethylammonio) propane-1-sulfonate hydrochloride in the same was as for Intermediate 27. MS (ESI) m/z 501.4 [M+]⁺. ¹H NMR (400 MHZ, DMSO-d₆): δ 8.10 (t, NH, 1H), 3.46-3.41 (m, 4H), 3.31-3.27 (m, 2H), 3.04 (s, 6H), 3.73 (t, 1H), 2.50-2.44 (t, 2H), 2.15-2.07 (m, 4H), 2.00-1.95 (m, 2H), 1.50-1.34 (m, 4H), 1.30-1.15 (m, 26H).

### Synthesis of Intermediate 29

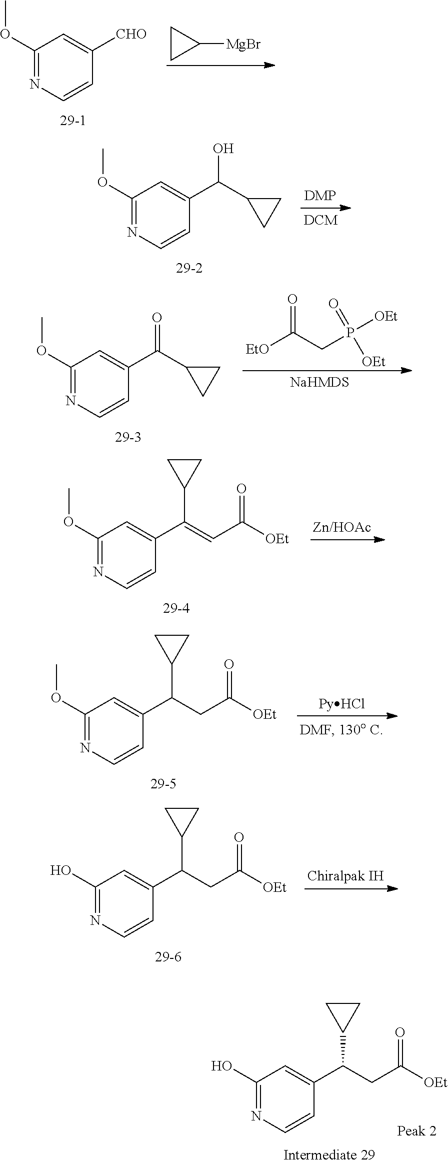

Step 1. To a solution of 2-methoxyisonicotinaldehyde (4.0 g, 29.2 mmol) in THF (50 mL) under nitrogen atmosphere was added cyclopropylmagnesium bromide (0.5 M, 70 mL, 35 mmol) dropwise at 0° C. The resulting mixture was stirred at 0° C. for 30 min and stirred for 30 min at r. t. Saturated aqueous ammonium chloride solution was added to the reaction mixture at 0° C., and the mixture was extracted with ethyl acetate. The extract was washed with water and saturated brine, and dried over anhydrous magnesium sulfate. After silica gel filtration, the solvent was evaporated under reduced pressure to give cyclopropyl(2-methoxypyridin-4-yl) methanol (29-2)(5.3 g, crude) as a brown oil. MS (ESI) m/z=180.2 [M+H]⁺.

Step 2. To a mixture of 29-2 (5.3 g, 29.2 mmol) in DCM (200 mL) was added DMP (13.85 g, 32.7 mmol) in portions at 0° C. The resulting mixture was stirred for 14 h at r. t. Solvent was removed and the residue was purified by silica-gel column chromatography (5% of EA in PE) to give cyclopropyl(2-methoxypyridin-4-yl) methanone (29-3)(3.9 g, yield: 75% over 2 steps) as a yellow oil. MS (ESI) m/z=178.1 [M+H]⁺.

Step 3. To a solution of ethyl diethylphosphonoacetate (9.81 g, 53.82 mmol) in THF (100 mL) at 0° C. was added NaHMDS (2.0 M, 22 mL) dropwise and stirred for 30 min at 0° C. Then to the above mixture was added 29-3 (3.9 g, 21.91 mmol) in THF (20 mL) dropwise at 0° C. After addition the resulting mixture was stirred for 30 min at r. t. and heated under reflux for 1 hr. Quenched with aq. NH₄Cl and separated, extracted with EA (50 mL×2). The organic phase was combined and washed with brine, dried and concentrated to give crude (E)-ethyl 3-cyclopropyl-3-(2-methoxypyridin-4-yl) acrylate (29-4)(10 g, crude, mixed with phosphonoacetate) as yellow oil. MS (ESI) m/z=248.1 [M+H]⁺

Step 4. To a solution of 29-4 (10 g, crude, 19.7 mmol) in acetic acid (100 mL) was added a zinc powder (12.8 g, 197 mmol, 10 eq.). The mixture was stirred at room temperature for 30 min. LCMS indicated the completion of reaction. The reaction mixture was then filtered, and the filtrate was evaporated under reduced pressure. The residue was purified by silica gel column chromatography (3% to 5% of EA in PE) to give ethyl 3-cyclopropyl-3-(2-methoxypyridin-4-yl) propanoate (29-5)(4.5 g, yield: 91% over 2 steps) as a colorless oil. MS (ESI) m/z=250.2 [M+H]⁺. ¹H NMR (400 MHZ, DMSO-d₆) δ 8.04 (d, 1H), 6.92-6.91 (m, 1H), 6.70 (s, 1H), 4.02-3.92 (m, 2H), 3.81 (s, 3H), 2.74 (d, 2H), 2.27-2.21 (m, 1H), 1.08 (t, 3H), 1.03-0.97 (m, 1H), 0.58-0.45 (m, 1H), 0.39-0.28 (m, 1H), 0.29-0.20 (m, 1H), 0.20-0.10 (m, 1H).

Step 5. To a solution of 29.5 (4.3 g, 17.27 mmol) in DMF (12 mL) was added pyridinium chloride (19.95 g, 172 mmol), and the mixture was stirred at 130° C. for 20 min. The reaction mixture was cooled to 0° C. and ethyl acetate (50 mL×2) was added. The resulting white precipitate was filtered off. The filtrate was evaporated under reduced pressure, and the residue was purified by silica gel column chromatography (EA to 5% of methanol in EA) to give ethyl 3-cyclopropyl-3-(2-hydroxypyridin-4-yl) propanoate (29-6) (2.5 g, yield: 61.6%) as a pale-yellow oil. MS (ESI) m/z=236.1 [M+H]⁺. ¹H NMR (400 MHz, DMSO-d₆) δ 11.40 (br, 1H), 7.26 (d, J=6.8 Hz, 1H), 6.17-6.14 (m, 2H), 4.05-3.98 (m, 2H), 2.68 (d, J=7.6 Hz, 2H), 2.08-2.02 (m, 1H), 1.11 (t, J=7.2 Hz, 3H), 1.03-0.86 (m, 1H), 0.58-0.43 (m, 1H), 0.28-0.10 (m, 2H).

Step 6. Desired product(S)-ethyl 3-cyclopropyl-3-(2-hydroxypyridin-4-yl) propanoate (Intermediate 29) was obtained by chiral separation (Method Info: Column: Chiralpak 1H, Mobile phase: Hex: EtOH=60:40, peak 2, Rt=11.193); The other enantiomer was eluted out first (peak 1, Rt=9.703).

### Synthesis of Intermediate 30

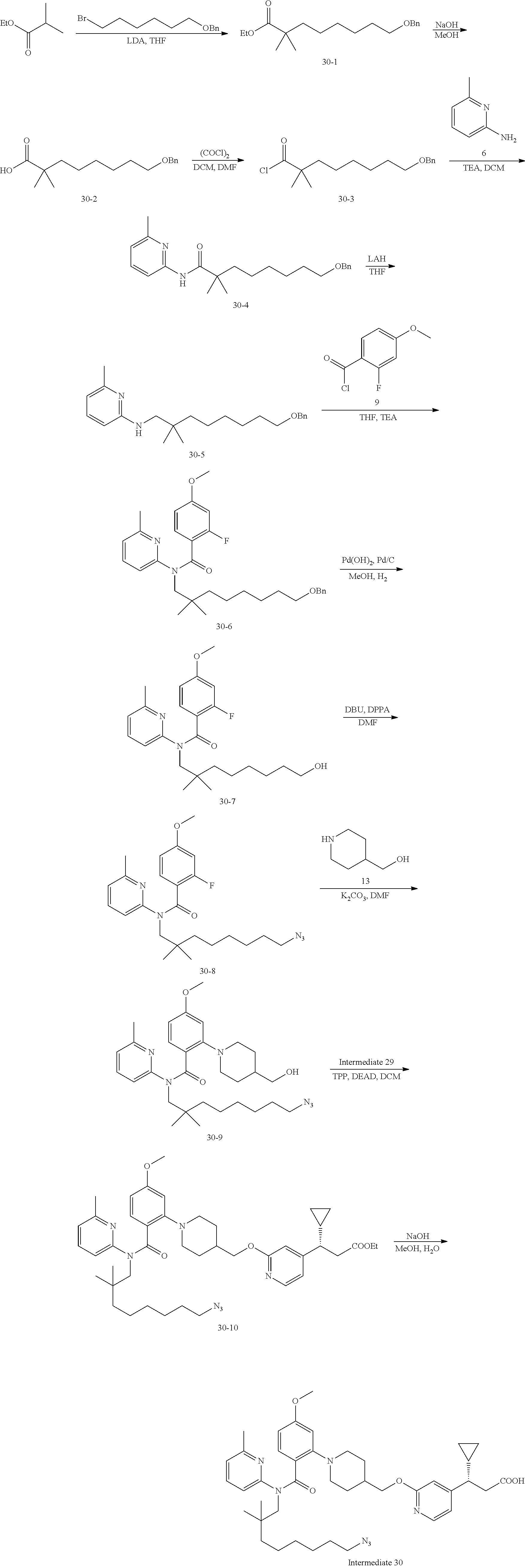

Step 1. To a solution of ethyl isobutyrate (200 mg, 1.72 mmol) in THF (2 mL) was added dropwise LDA (1.29 mL, 2.59 mmol, 2 M) at −70° C. under N₂ atmosphere. The reaction mixture was stirred at −70° C. for 1 h. Then to the mixture was added dropwise (((6-bromohexyl)oxy)methyl)benzene (700.9 mg, 2.59 mmol). The reaction mixture was warmed up to room temperature and stirred overnight. After the reaction was completed, the reaction mixture was quenched with NH₄Cl aq (10 mL). The resulting mixture was extracted with EA, dried with Na₂SO₄, filtered, and the filtrate was concentrated. The residue was purified by flash (EA/PE=0-10%) to give ethyl 8-(benzyloxy)-2,2-dimethyloctanoate (30-1)(410 mg, yield: 77.7%) as a colorless oil. ¹H NMR (400 MHZ, DMSO-d₆): 7.36-7.25 (m, 5H), 4.42 (s, 2H), 4.07-4.01 (m, 2H), 3.40 (t, 2H), 1.54-1.42 (m, 4H), 1.34-1.13 (m, 9H), 1.09 (s, 6H).

Step 2. To a solution of 30-1 (410 mg, 1.34 mmol) in MeOH (5 mL) and H₂O (1 mL) was added NaOH (160.8 mg, 4.02 mmol), and the mixture was stirred at 70° C. for 6 h. The reaction mixture was diluted with water and adjusted to pH=5 with HCl (2N). The resulting mixture was extracted with EA. The organic phase was washed with water and brine, dried with Na₂SO₄, filtered, and the filtrate was concentrated to give 8-(benzyloxy)-2,2-dimethyloctanoic acid (30-2)(250 mg, yield: 67.1%) as a colorless oil. ¹H NMR (400 MHz, DMSO-d₆): 11.96 (br, 1H), 7.36-7.25 (m, 5H), 4.43 (s, 2H), 3.40 (t, 2H), 1.55-1.16 (m, 10H), 1.06 (s, 6H).

Step 3. To a solution of 30-2 (250 mg, 0.90 mmol) in dry DCM (5 mL) was added dropwise (COCl)₂ (171 mg, 1.35 mmol) and DMF (1 drop) at 0° C. under nitrogen atmosphere. The reaction mixture was stirred at room temperature for 1 h. After the reaction was completed, the reaction mixture was concentrated to give 8-(benzyloxy)-2,2-dimethyloctanoyl chloride (30-3)(250 mg, yield: 93.8%) as a yellow oil.

Step 4. To a solution of 6-methylpyridin-2-amine (91.1 mg, 0.84 mmol) and TEA (170.3 mg, 1.69 mmol) in THF (5 mL) was added dropwise a solution of 30-3 (250 mg, 0.84 mmol) in THF (2 mL) at 0° C. under nitrogen atmosphere. The reaction mixture was warmed up to room temperature and stirred for 2 h. The reaction mixture was quenched with water and extracted with EA, dried with MgSO₄, filtered, and the filtrate was concentrated to give 8-(benzyloxy)-2,2-dimethyl-N-(6-methylpyridin-2-yl) octanamide (30-4)(300 mg, yield: 96.7%) as a yellow oil. MS (ESI) m/z 369.3 [M+H]⁺.

Step 5. To a solution of 30-4 (1 g, 2.72 mmol) in THF (10 mL) was added LAH (420 mg, 10.88 mmol) in portions at 0° C. The mixture was stirred at room temperature for 12 h. The reaction mixture was quenched with water (0.5 mL) at 0° C. Then 15% of NaOH aqueous solution (0.5 mL) and water (1.5 mL) were added. The resulting mixture was diluted with EA, dried with MgSO₄, filtered, and the filtrate was concentrated to give N-(8-(benzyloxy)-2,2-dimethyloctyl)-6-methylpyridin-2-amine (30-5)(0.90 g, yield: 93.8%) as a yellow oil. ¹H NMR (400 MHZ, DMSO-d₆): 7.36-7.25 (m, 5H), 7.21-7.17 (t, 1H), 6.33-6.31 (d, 1H), 6.27-6.25 (d, 1H), 6.10-6.07 (t, 1H), 4.43 (s, 2H), 3.41-3.38 (t, 2H), 3.08 (d, 2H), 2.21 (s, 3H), 1.54-1.47 (m, 2H), 1.36-1.16 (m, 8H), 0.85 (s, 6H). MS (ESI) m/z 355.3 [M+H]⁺.

Step 6. To a solution of 30-5 (4.7 g, 1.33 mol) and TEA (2.69 g, 2.66 mol) in THF (25 mL) was added dropwise a solution of 2-fluoro-4-methoxybenzoyl chloride (2.75 g, 1.46 mol) in THF (25 mL) at 0° C. under nitrogen atmosphere. The reaction mixture was warmed up to room temperature and stirred for 8 h. The reaction mixture was quenched with water and extracted with EA, dried with Na₂SO₄, filtered, and the filtrate was concentrated. The residue was purified by column chromatography on silica gel (EA/PE=0-20%) to give N-(8-(benzyloxy)-2,2-dimethyloctyl)-2-fluoro-4-methoxy-N-(6-methylpyridin-2-yl)benzamide (30-6)(4.1 g, yield: 61.0%) as a yellow oil. ¹H NMR (400 MHZ, DMSO-d₆): 7.44 (t, J=7.8 Hz, 1H), 7.36-7.25 (m, 5H), 7.10-7.05 (t, 1H), 6.95 (d, J=7.6 Hz, 1H), 6.74 (d, J=8.0 Hz, 1H), 6.67-6.62 (m, 2H), 4.43 (s, 2H), 3.99 (s, 2H), 3.70 (s, 3H), 3.37 (t, 2H), 2.34 (s, 3H), 1.49-1.42 (m, 2H), 1.23-1.13 (m, 2H), 1.12-0.93 (m, 6H), 0.78 (s, 6H). MS (ESI) m/z 507.3 [M+H]⁺.

Step 7. To a solution of 30-6 (2.0 g, 3.95 mmol) in MeOH (20 mL) was added Pd(OH)₂ (0.3 g) and Pd/C (0.3 g). The mixture was stirred at room temperature under H₂ for 12 h. After the reaction was completed, the reaction mixture was filtered, and the filtrate was concentrated to give 2-fluoro-N-(8-hydroxy-2,2-dimethyloctyl)-4-methoxy-N-(6-methylpyridin-2-yl)benz amide (30-7)(1.55 g, yield: 94.5%) as a colorless oil. MS (ESI) m/z 417.3 [M+H]⁺.

Step 8. To a solution of 30-7 (1.6 g, 3.85 mmol) in DMF (20 mL) was added DBU (1.17 g, 7.69 mmol) and DPPA (2.1 g, 7.69 mmol). The reaction mixture was quenched with water and extracted with EA. The organic phase was washed with water and brine, dried with Na₂SO₄, filtered, and the filtrate was concentrated. The residue was purified by column chromatography on silica gel (EA/PE=0-10%) to give N-(8-azido-2,2-dimethyloctyl)-2-fluoro-4-methoxy-N-(6-methylpyridin-2-yl)benzamide (30-8)(1.5 g, yield: 88.2%) as a colorless oil. ¹H NMR (400 MHZ, DMSO-d₆): 7.46 (t, 1H), 7.08 (t, 1H), 6.98 (d, 1H), 6.74 (d, 1H), 6.67-6.62 (m, 2H), 3.99 (s, 2H), 3.71 (s, 3H), 3.27 (t, 2H), 2.35 (s, 3H), 1.47-1.40 (m, 2H), 1.23-1.00 (m, 8H), 0.78 (s, 6H). MS (ESI) m/z 442.2 [M+H]⁺.

Step 9. To a solution of 30-8 (1.5 g, 3.40 mmol) in DMF (5 mL) was added piperidin-4-ylmethanol (1.56 g, 13.61 mmol), Cs₂CO₃ (2.22 g, 6.80 mmol) and Bu₄NI (0.25 g, 0.68 mmol). The mixture was stirred at 110° C. for 24 h. The reaction mixture was quenched with water and extracted with EA. The organic phase was washed with water and brine, dried with Na₂SO₄, filtered, and the filtrate was concentrated. The residue was purified by column chromatography on silica gel (EA/PE=0-35%) to give N-(8-azido-2,2-dimethyloctyl)-2-(4-(hydroxymethyl) piperidin-1-yl)-4-methoxy-N-(6-methylpyridin-2-yl)benzamide (30-9)(1.0 g, yield: 54.9%) as a colorless oil. ¹H NMR (400 MHZ, DMSO-d₆): 7.26 (t, J=7.6 Hz, 1H), 7.09 (d, J=8.4 Hz, 1H), 6.85 (d, J=7.6 Hz, 1H), 6.51-6.49 (m, 1H), 6.40 (br, 1H), 6.20 (s, 1H), 4.46 (br, 1H), 4.18-3.98 (m, 2H), 3.68 (s, 3H), 3.32-3.25 (m, 6H), 2.52-2.44 (m, 2H), 2.40-2.28 (m, 1H), 2.37 (s, 3H), 1.60-1.52 (m, 2H), 1.50-1.40 (m, 2H), 1.38-1.30 (m, 2H), 1.25-0.89 (m, 8H), 0.75 (s, 6H). MS (ESI) m/z 537.4 [M+H]⁺.

Step 10. To a mixture of 30-9 (250 mg, 0.47 mmol), Intermediate 29 (131.5 mg, 0.56 mmol) and TPP (244.4 mg, 0.93 mmol) in DCM (2 mL) was added DEAD (162.3 mg, 0.93 mmol) dropwise under nitrogen atmosphere at 0° C. The mixture was stirred at room temperature for 12 h. After the reaction was completed, the reaction mixture was quenched with water and extracted with DCM. The organic phase was washed with water and brine, dried with Na₂SO₄, filtered, and the filtrate was concentrated. The residue was purified by column chromatography on silica gel (EA/PE=0-25%) to give (R)-ethyl 3-(2-((1-(2-((8-azido-2,2-dimethyloctyl)(6-methylpyridin-2-yl) carbamoyl)-5-methoxyphenyl) piperidin-4-yl)methoxy)pyridin-4-yl)-3-cyclopropylpropanoate (30-10)(90 mg, yield: 25.6%) as a colorless oil. MS (ESI) m/z 754.5 [M+H]⁺.

Step 11. To a solution of 30-10 (90 mg, 0.12 mmol) in MeOH (2 mL) and H₂O (0.4 mL) was added NaOH (14.3 mg, 0.36 mmol), and the mixture was stirred at 45° C. for 2 h. After the reaction was completed, the reaction mixture was diluted with water and adjusted to pH=5 with HCl (2N). The resulting mixture was extracted with EA. The organic phase was washed with water and brine, dried with Na₂SO₄, filtered, and the filtrate was concentrated to give (R)-3-(2-((1-(2-((8-azido-2,2-dimethyloctyl)(6-methylpyridin-2-yl) carbamoyl)-5-methoxyphenyl) piperidin-4-yl)methoxy)pyridin-4-yl)-3-cyclopropylpropanoic acid (Intermediate 30)(85 mg, yield: 98.0%) as a colorless gum. ¹H NMR (400 MHZ, DMSO-d₆): 8.05-8.04 (d, 1H), 7.28 (br, 1H), 7.12-7.10 (d, 1H), 6.93-6.92 (d, 1H), 6.87-6.86 (d, 1H), 6.72 (s, 1H), 6.53-6.51 (d, 1H), 6.44 (br, 1H), 6.23 (s, 1H), 4.14-4.10 (m, 4H), 3.69 (s, 3H), 3.28-3.24 (t, 3H), 2.69-2.68 (d, 2H), 2.37 (s, 3H), 2.27-2.21 (m, 1H), 1.81-1.69 (br, 1H), 1.69-1.61 (m, 2H), 1.45-1.38 (m, 2H), 1.25-1.08 (m, 6H), 1.06-0.90 (m, 5H), 0.76 (s, 6H), 0.52-0.50 (m, 1H), 0.35-0.27 (m, 2H), 0.18-0.17 (m, 1H). MS (ESI) m/z 726.6 [M+H]⁺.

### Synthesis of Intermediate 31

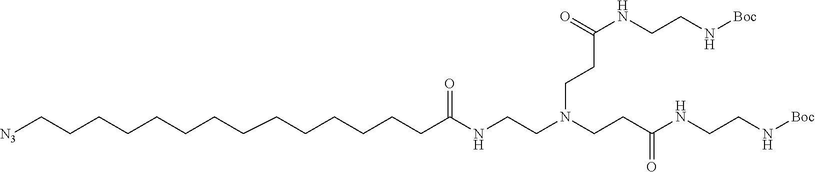 [SMILES: CC(C)(C)OC(=O)NCCNC(=O)CCN(CCNC(=O)CCCCCCCCCCCCCCN=[N+]=[N-])CCC(=O)NCCNC(=O)OC(C)(C)C]

MS (ESI) m/z 754.5 [M+H]⁺. ¹H NMR (400 MHZ, DMSO-d₆) δ: 7.93 (brs, 2H), 7.62 (br, 1H), 6.81 (br, 2H), 3.36-3.32 (t, 2H), 3.12-3.06 (m, 6H), 3.02-2.97 (m, 4H), 2.70-2.64 (m, 4H), 2.45-2.40 (m, 2H), 2.23-2.18 (m, 4H), 2.10-2.04 (t, 2H), 1.60-1.42 (m, 4H), 1.40 (s, 18H), 1.35-1.20 (m, 20H).

### Synthesis of Intermediate 32

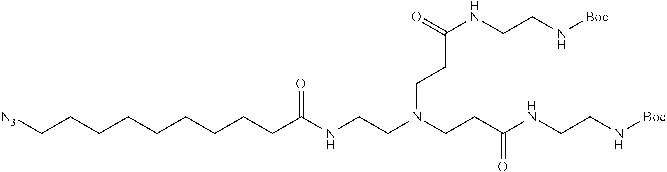 [SMILES: CC(C)(C)OC(=O)NCCNC(=O)CCN(CCNC(=O)CCCCCCCCCN=[N+]=[N-])CCC(=O)NCCNC(=O)OC(C)(C)C]

MS (ESI) m/z 684.5 [M+H]⁺. ¹H NMR (400 MHZ, DMSO-d₆) δ: 7.90 (br, NH, 2H), 7.69 (br, NH, 1H), 6.77 (br, NH, 2H), 3.35-3.30 (t, 2H), 3.08-3.04 (m, 6H), 3.00-2.96 (m, 4H), 2.64-2.58 (m, 4H), 2.45-2.40 (m, 2H), 2.20-2.15 (m, 4H), 2.07-2.00 (t, 2H), 1.54-1.48 (m, 4H), 1.37 (s, 18H), 1.30-1.20 (m, 10H).

### Synthesis of Intermediate 33

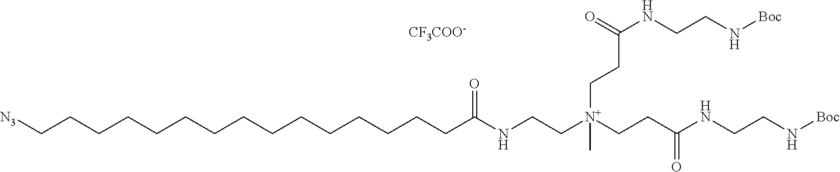 [SMILES: CC(C)(C)OC(=O)NCCNC(=O)CC[N+](C)(CCNC(=O)CCCCCCCCCCCCCCCN=[N+]=[N-])CCC(=O)NCCNC(=O)OC(C)(C)C ; "CF3COO-"]

MS (ESI) m/z 782.6 [M]⁺. ¹H NMR (400 MHZ, DMSO-d₆) δ: 8.16 (br, NH, 2H), 8.09 (br, NH, 1H), 6.83 (br, NH, 2H), 3.57-3.53 (m, 4H), 3.45-3.43 (m, 2H), 3.32-3.27 (m, 4H), 3.10-3.06 (m, 4H), 3.01 (s, 3H), 2.99-2.97 (m, 4H), 2.64-2.55 (m, 4H), 2.10-2.06 (t, 2H), 1.53-1.47 (m, 4H), 1.38 (s, 18H), 1.30-1.20 (m, 22H).

### Synthesis of Intermediate 34

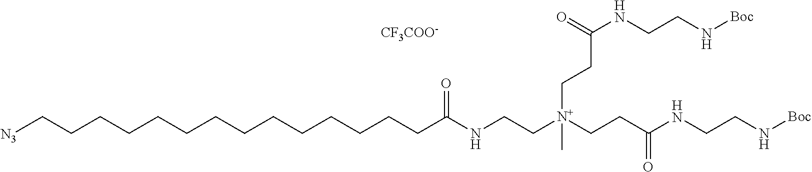 [SMILES: CC(C)(C)OC(=O)NCCNC(=O)CC[N+](C)(CCNC(=O)CCCCCCCCCCCCCCN=[N+]=[N-])CCC(=O)NCCNC(=O)OC(C)(C)C ; "CF3COO-"]

MS (ESI) m/z 768.6 [M]⁺. ¹H NMR (400 MHZ, DMSO-d₆) δ: 8.19 (br, NH, 2H), 8.12 (br, NH, 1H), 6.86 (br, NH, 2H), 3.60-3.54 (m, 4H), 3.45-3.42 (m, 2H), 3.32-3.27 (m, 4H), 3.12-3.06 (m, 4H), 3.04 (s, 3H), 3.02-2.97 (m, 4H), 2.64-2.58 (m, 4H), 2.13-2.09 (t, 2H), 1.54-1.47 (m, 4H), 1.41 (s, 18H), 1.30-1.20 (m, 20H).

### Synthesis of Intermediate 35

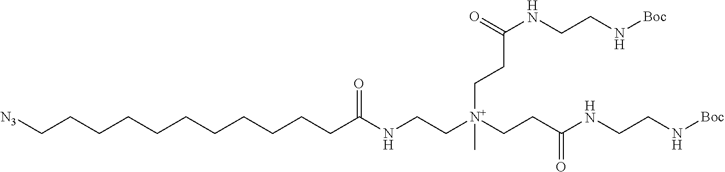 [SMILES: CC(C)(C)OC(=O)NCCNC(=O)CC[N+](C)(CCNC(=O)CCCCCCCCCCCN=[N+]=[N-])CCC(=O)NCCNC(=O)OC(C)(C)C]

MS (ESI) m/z 726.4 [M]⁺. ¹H NMR (400 MHZ, DMSO-d₆) δ: 8.16 (br, NH, 2H), 8.10 (br, NH, 1H), 6.82 (br, NH, 2H), 3.57-3.53 (m, 4H), 3.45-3.43 (m, 2H), 3.33-3.29 (m, 4H), 3.10-3.06 (m, 4H), 3.01 (s, 3H), 3.01-2.95 (m, 4H), 2.64-2.60 (m, 4H), 2.11-2.06 (t, 2H), 1.54-1.48 (m, 4H), 1.38 (s, 18H), 1.28-1.23 (m, 14H).

### Synthesis of Intermediate 36

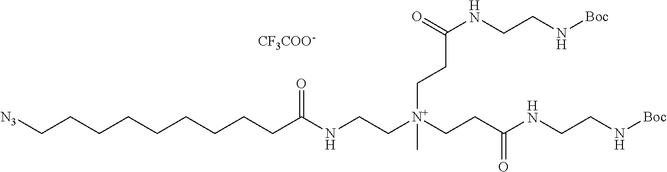 [SMILES: CC(C)(C)OC(=O)NCCNC(=O)CC[N+](C)(CCNC(=O)CCCCCCCCCN=[N+]=[N-])CCC(=O)NCCNC(=O)OC(C)(C)C ; "CF3COO-"]

MS (ESI) m/z 698.5 [M]⁺. ¹H NMR (400 MHZ, DMSO-d₆) δ: 8.15 (br, NH, 2H), 8.08 (br, NH, 1H), 6.82 (br, NH, 2H), 3.58-3.53 (m, 4H), 3.44-3.40 (m, 2H), 3.35-3.30 (m, 4H), 3.10-3.04 (m, 4H), 3.00 (s, 3H), 3.02-2.95 (m, 4H), 2.64-2.58 (m, 4H), 2.11-2.06 (t, 2H), 1.54-1.48 (m, 4H), 1.41 (s, 18H), 1.30-1.20 (m, 10H).

### Synthesis of Intermediate 37

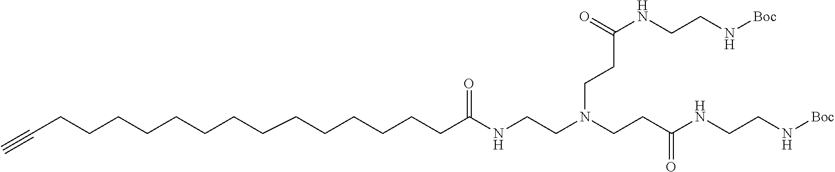 [SMILES: C#CCCCCCCCCCCCCCCC(=O)NCCN(CCC(=O)NCCNC(=O)OC(C)(C)C)CCC(=O)NCCNC(=O)OC(C)(C)C]

MS (ESI) m/z 737.6 [M+H]⁺. ¹H NMR (400 MHz, DMSO-d₆) δ: 7.92 (t, NH, 2H), 7.61 (t, NH, 1H), 6.79 (t, NH, 2H), 3.10-3.05 (m, 6H), 3.00-2.94 (m, 4H), 2.76-2.74 (t, 1H), 2.69-2.64 (m, 4H), 2.46-2.42 (m, 2H), 2.20-2.12 (m, 6H), 2.09-2.04 (t, 2H), 1.49-1.30 (m, 4H), 1.37 (s, 18H), 1.30-1.20 (m, 20H).

### Synthesis of Intermediate 38

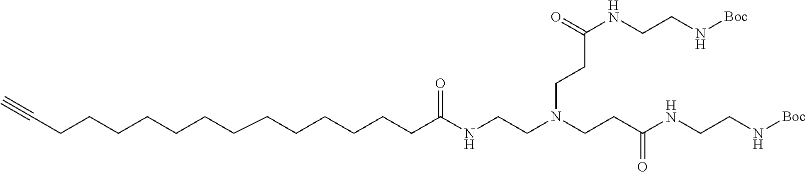 [SMILES: C#CCCCCCCCCCCCCCC(=O)NCCN(CCC(=O)NCCNC(=O)OC(C)(C)C)CCC(=O)NCCNC(=O)OC(C)(C)C]

MS (ESI) m/z 723.5 [M+H]⁺. ¹H NMR (400 MHZ, CDCl₃) δ: 7.88 (t, NH, 2H), 7.57 (t, NH, 1H), 6.75 (t, NH, 2H), 3.10-3.03 (m, 6H), 2.98-2.95 (m, 4H), 2.72-2.71 (t, 1H), 2.64-2.59 (m, 4H), 2.43-2.39 (m, 2H), 2.19-2.11 (m, 6H), 2.05-2.01 (t, 2H), 1.48-1.30 (m, 4H), 1.38 (s, 18H), 1.30-1.20 (m, 18H).

### Synthesis of Intermediate 39

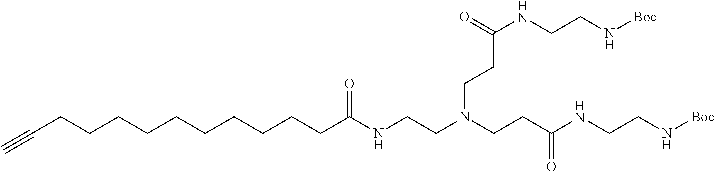 [SMILES: C#CCCCCCCCCCCC(=O)NCCN(CCC(=O)NCCNC(=O)OC(C)(C)C)CCC(=O)NCCNC(=O)OC(C)(C)C]

MS (ESI) m/z 681.4 [M+H]⁺. ¹H NMR (400 MHZ, DMSO-d₆) δ: 7.90 (t, NH, 2H), 7.58 (t, NH, 1H), 6.77 (t, NH, 2H), 3.12-3.04 (m, 6H), 3.00-2.94 (m, 4H), 2.72-2.70 (t, 1H), 2.63-2.57 (m, 4H), 2.42-2.39 (m, 2H), 2.16-2.10 (m, 6H), 2.06-2.00 (m, 2H), 1.44-1.32 (m, 4H), 1.37 (s, 18H), 1.30-1.20 (m, 12H).

### Synthesis of Intermediate 40

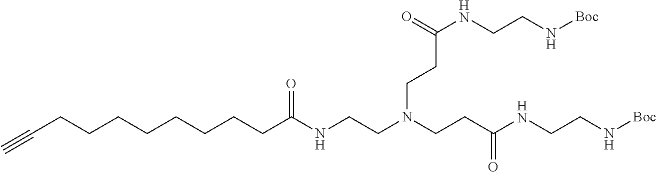 [SMILES: C#CCCCCCCCCC(=O)NCCN(CCC(=O)NCCNC(=O)OC(C)(C)C)CCC(=O)NCCNC(=O)OC(C)(C)C]

MS (ESI) m/z 653.5 [M+H]⁺. ¹H NMR (400 MHZ, DMSO-d₆) δ: 7.90 (t, NH, 2H), 7.59 (t, NH, 1H), 6.77 (t, NH, 2H), 3.10-3.05 (m, 6H), 3.00-2.94 (m, 4H), 2.73-2.71 (t, 1H), 2.63-2.57 (m, 4H), 2.42-2.39 (m, 2H), 2.17-2.10 (m, 6H), 2.06-2.01 (t, 2H), 1.49-1.32 (m, 4H), 1.37 (s, 18H), 1.28-1.20 (m, 8H).

### Synthesis of Intermediate 41

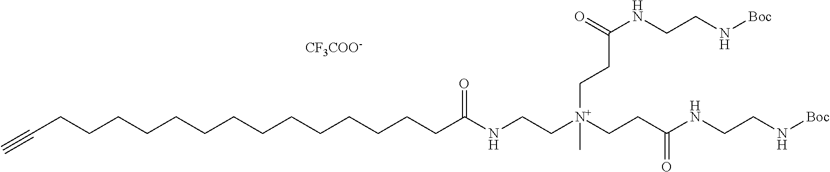 [SMILES: C#CCCCCCCCCCCCCCCC(=O)NCC[N+](C)(CCC(=O)NCCNC(=O)OC(C)(C)C)CCC(=O)NCCNC(=O)OC(C)(C)C ; "CF3COO-"]

MS (ESI) m/z 751.6 [M]⁺. ¹H NMR (400 MHZ, DMSO-d₆) ¿: 8.18 (t, NH, 2H), 8.12 (t, NH, 1H), 6.82 (t, NH, 2H), 3.57-3.53 (m, 4H), 3.45-3.42 (m, 2H), 3.30-3.26 (m, 2H), 3.11-3.07 (m, 4H), 3.01 (s, 3H), 3.01-2.97 (m, 4H), 2.72-2.70 (t, 1H), 2.65-2.60 (m, 4H), 2.16-2.07 (m, 4H), 1.50-1.30 (m, 4H), 1.38 (s, 18H), 1.30-1,20 (m, 20H).

### Synthesis of Intermediate 42

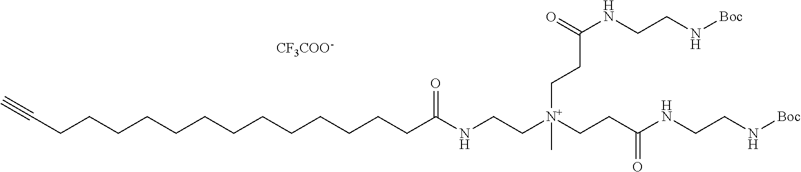 [SMILES: C#CCCCCCCCCCCCCCC(=O)NCC[N+](C)(CCC(=O)NCCNC(=O)OC(C)(C)C)CCC(=O)NCCNC(=O)OC(C)(C)C ; "CF3COO-"]

MS (ESI) m/z 737.6 [M]⁺. ¹H NMR (400 MHz, DMSO-d₆) δ: 8.19 (t, NH, 2H), 8.12 (t, NH, 1H), 6.86 (t, NH, 2H), 3.57-3.53 (m, 4H), 3.50-3.46 (m, 2H), 3.33-3.29 (m, 2H), 3.13-3.09 (m, 4H), 3.04 (s, 3H), 3.04-2.98 (m, 4H), 2.77-2.75 (t, 1H), 2.67-2.62 (m, 4H), 2.19-2.10 (m, 4H), 1.46-1.30 (m, 4H), 1.42 (s, 18H), 1.30-1,20 (m, 18H).

### Synthesis of Intermediate 43

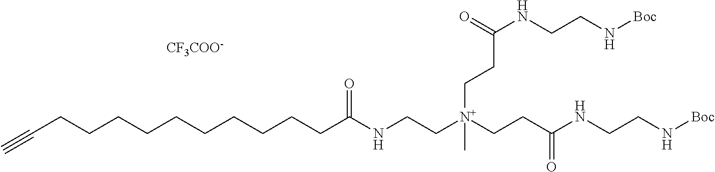 [SMILES: C#CCCCCCCCCCCC(=O)NCC[N+](C)(CCC(=O)NCCNC(=O)OC(C)(C)C)CCC(=O)NCCNC(=O)OC(C)(C)C ; "CF3COO-"]

MS (ESI) m/z 695.5 [M]⁺. ¹H NMR (400 MHZ, DMSO-d₆) δ: 8.22 (t, NH, 2H), 8.17 (t, NH, 1H), 6.84 (t, NH, 2H), 3.57-3.53 (m, 4H), 3.50-3.46 (m, 2H), 3.30-3.25 (m, 2H), 3.12-3.09 (m, 4H), 3.04 (s, 3H), 3.04-2.98 (m, 4H), 2.77-2.75 (t, 1H), 2.67-2.62 (m, 4H), 2.19-2.10 (m, 4H), 1.46-1.30 (m, 4H), 1.42 (s, 18H), 1.30-1,20 (m, 12H).

### Synthesis of Intermediate 44

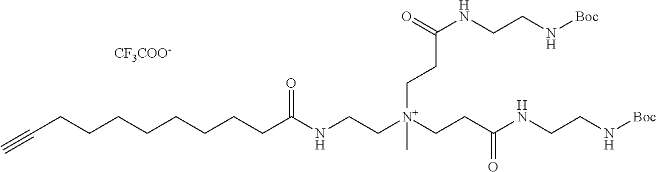 [SMILES: C#CCCCCCCCCC(=O)NCC[N+](C)(CCC(=O)NCCNC(=O)OC(C)(C)C)CCC(=O)NCCNC(=O)OC(C)(C)C ; "CF3COO-"]

MS (ESI) m/z 667.5 [M]⁺. ¹H NMR (400 MHZ, DMSO-d₆): 8.34 (t, NH, 2H), 8.17 (t, NH, 1H), 6.83 (t, NH, 2H), 3.57-3.53 (m, 4H), 3.50-3.46 (m, 2H), 3.32-3.28 (m, 2H), 3.12-3.09 (m, 4H), 3.01 (s, 3H), 3.01-2.97 (m, 4H), 2.74-2.72 (t, 1H), 2.65-2.60 (m, 4H), 2.14-2.06 (m, 4H), 1.49-1.30 (m, 4H), 1.39 (s, 18H), 1.30-1,20 (m, 8H).

### Synthesis of Intermediate 45

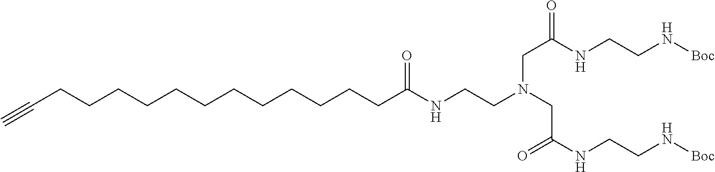 [SMILES: C#CCCCCCCCCCCCCC(=O)NCCN(CC(=O)NCCNC(=O)OC(C)(C)C)CC(=O)NCCNC(=O)OC(C)(C)C]

MS (ESI) m/z 681.5 [M]⁺. ¹H NMR (400 MHZ, DMSO-d₆) δ: 7.52 (t, NH, 2H), 6.85 (t, NH, 1H), 5.31 (t, NH, 2H), 3.39 (br, 4H), 3.30 (br, 6H), 3.21 (br, 4H), 2.64 (br, 2H), 2.27-2.23 (t, 2H), 2.20-2.16 (dt, 2H), 1.94-1.93 (t, 1H), 1.56-1.48 (m, 2H), 1.41 (s, 18H), 1.40-1.30 (m, 18H).

### Synthesis of Intermediate 46

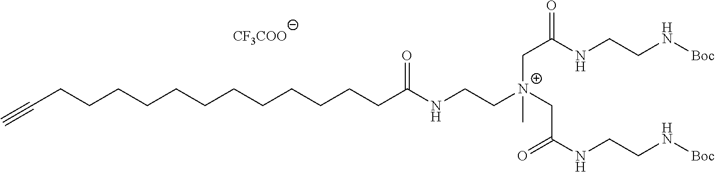 [SMILES: C#CCCCCCCCCCCCCC(=O)NCC[N+](C)(CC(=O)NCCNC(=O)OC(C)(C)C)CC(=O)NCCNC(=O)OC(C)(C)C ; "CF3COO"]

MS (ESI) m/z 695.6 [M]⁺. ¹H NMR (400 MHZ, DMSO-d₆) δ: 8.63-8.61 (t, NH, 2H), 8.11-8.08 (t, NH, 1H), 6.86-6.83 (t, NH, 2H), 4.28 (s, 4H), 3.74-3.70 (m, 2H), 3.53-3.48 (m, 2H), 3.38 (s, 3H), 3.17-3.11 (m, 4H), 3.04-3.00 (m, 4H), 2.73-2.71 (t, 1H), 2.16-2.11 (m, 2H), 2.10-2.06 (t, 2H), 1.48-1.30 (m, 4H), 1.40 (s, 18H), 1.28-1.19 (m, 16H).

### Synthesis of Intermediate 47

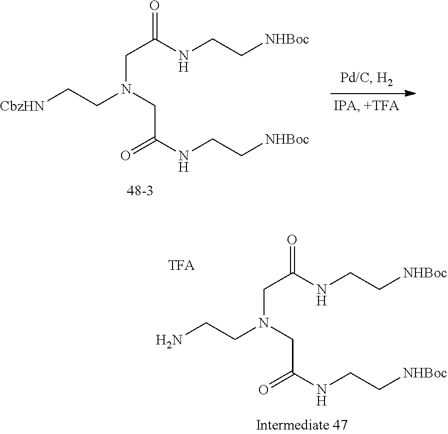 [SMILES: CC(C)(C)OC(=O)NCCNC(=O)CN(CCNC(=O)OCc1ccccc1)CC(=O)NCCNC(=O)OC(C)(C)C ; CC(C)(C)OC(=O)NCCNC(=O)CN(CCN)CC(=O)NCCNC(=O)OC(C)(C)C]

A flask charged with 48-3 (700 mg, 1.178 mmol) and Pd/C (wet, ˜150 mg) in isopropyl alcohol (40 mL) containing TFA (200 mg) was degassed and filled with hydrogen using a balloon. The resulting mixture was then hydrogenated for 1.5 hrs at room temperature. The mixture was filtered over celite, washed with EA and IPA. The filtrate was combined and concentrated to give crude di-tert-butyl(((2,2′-((2-aminoethyl) azanediyl)bis(acetyl)) bis(azanediyl)) \bis(ethane-2,1-diyl))dicarbamate, Intermediate 47, (550 mg, yield: 100%) as a white gum which was directly used in the next step. MS (ESI) m/z 461.3 [M+H]⁺. ¹H NMR (400 MHZ, DMSO-d₆): 7.84 (s, 1H), 7.75 (s, 1H), 6.81 (s, 1H), 6.73 (s, 1H), 3.30-3.17 (m, 2H), 3.14-3.08 (m, 2H), 3.04-2.98 (m, 6H), 2.95-2.89 (m, 2H), 2.58 (t, J=5.6 Hz, 2H), 2.53 (t, J=6.4 Hz, 2H), 1.37 (s, 18H).

### Synthesis of Intermediate 48

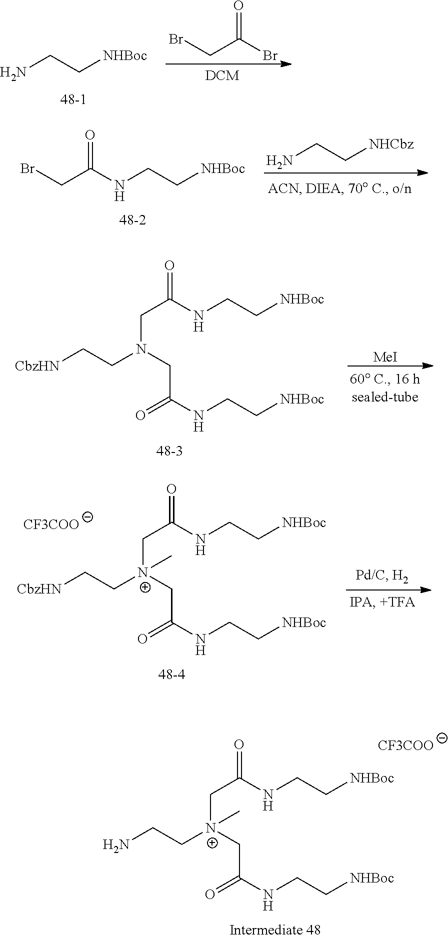

Step 1. To a mixture of tert-butyl(2-aminoethyl) carbamate (20 g, 0.125 mol) and TEA (18.95 g, 0.187 mol) in DCM (200 mL) at 0° C. was added dropwise 2-bromoacetyl bromide (30 g, 0.15 mol) in DCM (30 mL). The resulting mixture was stirred for 12 hrs at room temperature, followed by addition of with water (100 mL) and extraction with DCM. The combined organic phase was dried and concentrated. The residue was purified by silica gel column chromatography (PE/EA=5:1 to 2:1) to give tert-butyl(2-(2-bromoacetamido)ethyl) carbamate, 48-2, (9.5 g, yield: 27%) as pale yellow solid. MS (ESI) m/z 303.1 [M+Na]. ¹H NMR (400 MHZ, DMSO-d₆): 8.26 (s, 1H), 6.80 (s, 1H), 3.83 (s, 2H), 3.12-3.07 (m, 2H), 3.00-2.96 (m, 2H), 1.38 (s, 9H).

Step 2. A mixture of 48-2 (9.1 g, 32.5 mmol), benzyl(2-aminoethyl) carbamate (2.4 g, 12.3 mmol) and DIEA (6.68 g, 51.3 mmol) in MeCN(100 mL) was heated at 70° C. for 16 hrs. The product benzyl(11-(2-((2-((tert-butoxycarbonyl)amino)ethyl)amino)-2-oxoethyl)-2,2-dimethyl-4,9-dioxo-3-oxa-5,8,11-triazatridecan-13-yl) carbamate, 48-3, was collected by filtration as white solid (5.6 g, yield: 76%). MS (ESI) m/z 595.4 [M+H]. ¹H

NMR (400 MHZ, DMSO-d₆): 8.03 (s, 2H), 7.35-7.30 (m, 5H), 7.21 (s, 1H), 6.80 (s, 2H), 5.01 (s, 2H), 3.18 (s, 4H), 3.11-3.08 (m, 4H), 3.01-2.98 (m, 4H), 2.55-2.53 (m, 2H), 1.37 (s, 18H).

Step 3. A pressure tube charged with 48-3 was collected by filtration as white solid (600 mg, 1.01 mmol) and Mel (2 mL, 32 mmol) in ACN(7 mL) was sealed and heated at 60° C. for 20 hrs. The reaction mixture was concentrated and the residue was purified by prep-HPLC to give N-(2-(((benzyloxy) carbonyl)amino)ethyl)-2-((2-((tert-butoxycarbonyl)amino)ethyl)amino)-N-(2-((2-((tert-butoxycarbonyl)amino)ethyl)amino)-2-oxoethyl)-N-methyl-2-oxoethan −1-aminium 2,2,2-trifluoroacetate, 48-4, (490 mg, yield: 81% yield) as a white solid after freeze drying. MS (ESI) m/z 609.3 [M+]. ¹H NMR (400 MHZ, DMSO-d₆): 8.64 (t, 2H), 7.58 (s, tH), 7.39-7.32 (m, 5H), 6.84 (mt, 2H), 5.05 (s, 2H), 4.29 (s, 4H), 3.79-3.75 (m, 2H), 3.51-3.46 (m, 2H), 3.34 (s, 3H), 3.13-3.10 (m, 4H), 3.03-3.00 (m, 4H), 1.37 (s, 18H).

Step 4. A flask charged with 48-4 (520 mg, 0.85 mmol) and Pd/C (wet, ˜100 mg) in isopropyl alcohol (25 mL) was degassed and filled with hydrogen using a balloon. The resulting mixture was then hydrogenated for 1.5 hrs at r. t. The mixture was filtered over celite, washed with EA and IPA. The filtrate was combined and concentrated to give crude N-(2-aminoethyl)-2-((2-((tert-butoxycarbonyl)amino)ethyl)amino)-N-(2-((2-((tert-butoxycarbonyl)amino)ethyl)amino)-2-oxoethyl)-N-methyl-2-oxoethan −1-aminium 2,2,2-trifluoroacetate, Intermediate 48, (490 mg, crude) as a pale-yellow gum which was directly used in the next step. Note: TFA (200 mg, 1.75 mmol) was added to stabilize the product before hydrogenation.

### Synthesis of Intermediate 49

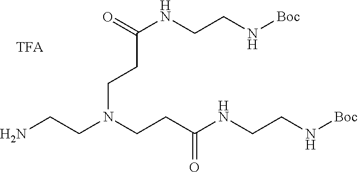 [SMILES: CC(C)(C)OC(=O)NCCNC(=O)CCN(CCN)CCC(=O)NCCNC(=O)OC(C)(C)C]

Intermediate 49 is the intermediate 15-8 in the preparation of Intermediate 15.

### Synthesis of Intermediate 50

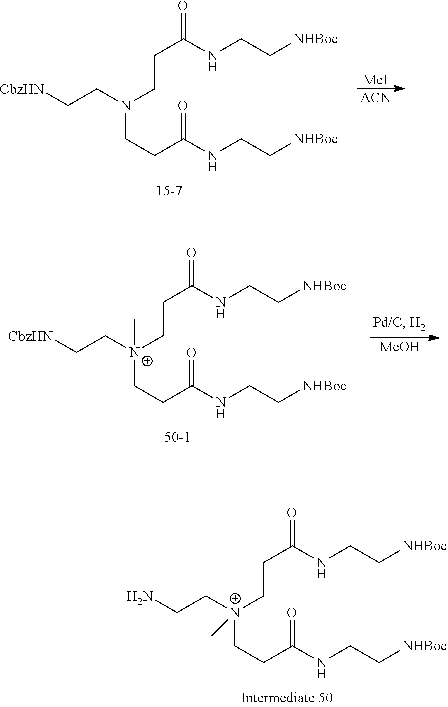 [SMILES: "MeI" ; "MeOH" ; CC(C)(C)OC(=O)NCCNC(=O)CCN(CCNC(=O)OCc1ccccc1)CCC(=O)NCCNC(=O)OC(C)(C)C ; "ACN" ; CC(C)(C)OC(=O)NCCNC(=O)CC[N+](C)(CCNC(=O)OCc1ccccc1)CCC(=O)NCCNC(=O)OC(C)(C)C ; CC(C)(C)OC(=O)NCCNC(=O)CC[N+](C)(CCN)CCC(=O)NCCNC(=O)OC(C)(C)C]

Step 1. A pressure tube charged with a suspension of benzyl(12-(3-((2-((tert-butoxycarbonyl)amino)ethyl)amino)-3-oxopropyl)-2,2-dimethyl-4,9-dioxo-3-oxa-5,8,12-triazatetradecan-14-yl) carbamate (500 mg, 0.80 mmol) and Mel (2 mL, 32 mmol) in ACN(5 mL) was sealed and heated at 60° C. for 20 hrs. The reaction mixture was concentrated and the residue was purified by prep-HPLC (TFA method) to give N-(2-(((benzyloxy) carbonyl)amino)ethyl)-3-((2-((tert-butoxycarbonyl)amino)ethyl)amino)-N-(3-((2-((tert-butoxycarbonyl)amino)ethyl)amino)-3-oxopropyl)-N-methyl-3-oxopropan-1-aminium 2,2,2-trifluoroacetate, 50-1, (450 mg, 97% yield) as a white solid. ¹H NMR (400 MHz, DMSO-d₆): 8.15 (t, 1H), 7.60-7.55 (t, 1H), 7.39-7.31 (m, 5H), 6.83-6.80 (t, 2H), 5.06 (s, 2H), 3.58-3.54 (m, 4H), 3.44-3.42 (m, 2H), 3.35-3.33 (m, 2H), 3.10-3.07 (m, 4H), 3.01 (s, 3H), 3.01-2.98 (m, 4H), 2.63-2.60 (m, 4H), 1.37 (s, 18H). MS (ESI) m/z 637.4 [M+].

Step 2. A flask charged with 50-1 (450 mg, 0.71 mmol) and Pd/C (wet, ˜100 mg) in MeOH (25 mL) was degassed and filled with hydrogen using a balloon. The resulting mixture was then hydrogenated for 2 hrs at room temperature. The mixture was filtered over celite, washed with EA and MeOH. The filtrate was combined and concentrated to give crude N-(2-aminoethyl)-3-((2-((tert-butoxycarbonyl)amino)ethyl)amino)-N-(3-((2-((tert-butoxy carbonyl)amino)ethyl) amino)-3-oxopropyl)-N-methyl-3-oxopropan-1-aminium 2,2,2-trifluoroacetate, Intermediate 50, (300 mg, 84% yield) as a yellow oil. ¹H NMR (400 MHZ, DMSO-d₆): 8.18 (t, 2H), 6.82 (t, 2H), 3.58-3.54 (m, 4H), 3.36-3.30 (m, 2H), 3.16-3.10 (m, 2H), 3.09-3.06 (m, 4H), 3.03 (s, 3H), 3.01-2.97 (m, 4H), 2.64-2.61 (m, 4H), 1.37 (m, 18H). MS (ESI) m/z 503.4 [M+].

### Synthesis of Intermediate 51

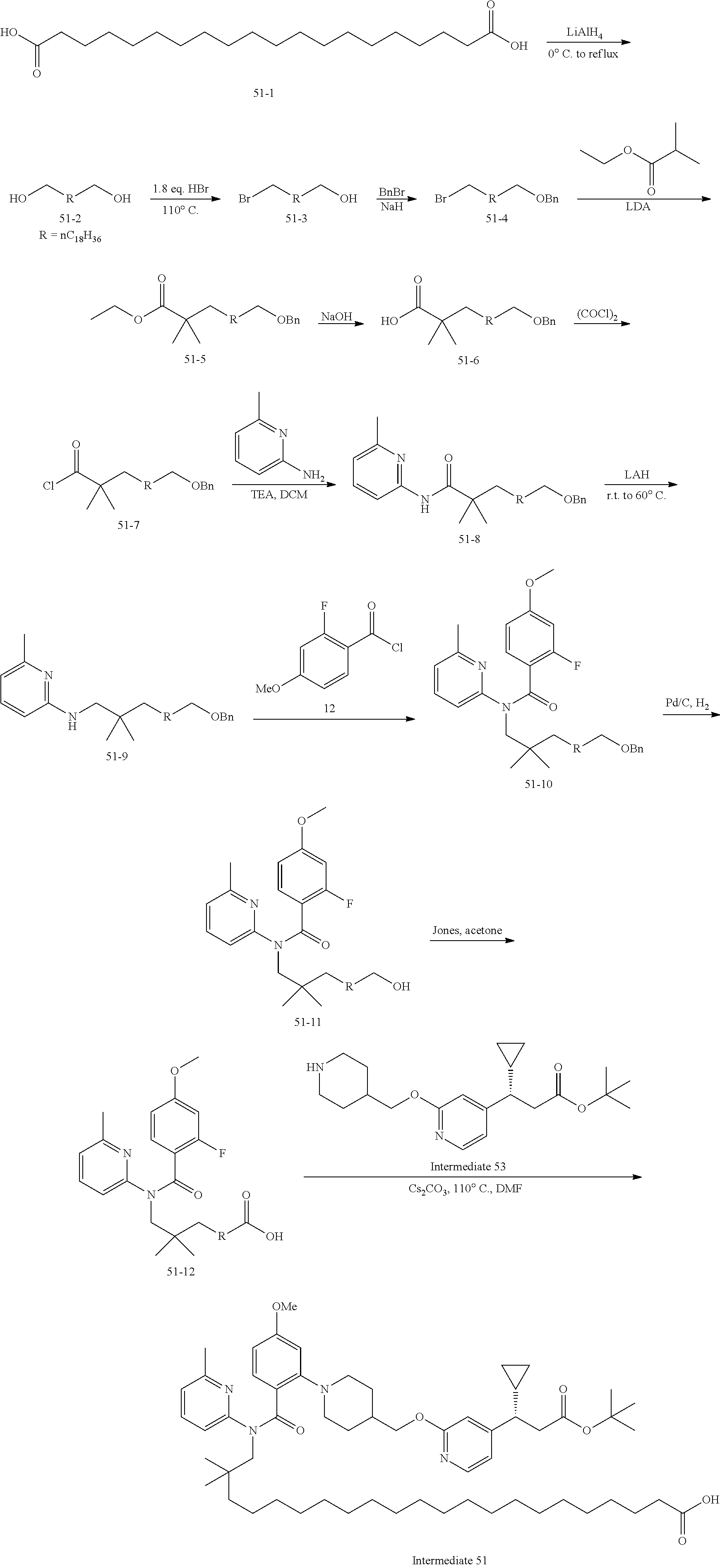

Step 1. To a solution of icosanedioic acid (6.9 g, 20.2 mmol) in THF (300 mL) was added LiAlH₄ (3.07 g, 80.8 mmol) in portions at 0° C. The mixture was stirred at 80° C. overnight under N₂. Water (3.1 mL) was added to the reaction at 0° C. followed by NaOH (15% solution, 3.1 mL) and more water (9.3 mL). The resulting mixture was diluted with THF, dried with Na₂SO₄, filtered, and the filtrate was concentrated to give icosane-1,20-diol, 51-2, (10.2 g, 81% yield) as a white solid. ¹H NMR (400 MHz, CDCl₃) δ 3.64 (t, 4H), 1.57 (br, 4H), 1.43-1.25 (m, 32H).

Step 2. To a solution of 51-2 (8.38 g, 26.7 mmol) in toluene (60 mL) was added HBr (9.01 g, 53.4 mmol). The reaction mixture was stirred at 110° C. overnight under N₂. The reaction mixture was concentrated under reduced pressure to remove excess reagent and solvent. The crude product was purified by column chromatograph (PE/EA=4/1) to give 20-bromoicosan-1-ol, 51-3, (4.4 g, 44% yield) as a white solid. ¹H NMR (400 MHZ, CDCl₃) δ 3.64 (t, 2H), 3.41 (t, 2H), 1.85 (m, 2H), 1.44-1.40 (m, 2H), 1.40-1.26 (m, 32H).

Step 3. To a solution of 51-3 (4.4 g, 11.75 mmol) in THF (20 mL) was added NaH (338.8 mg, 23.49 mmol) at 0° C. The reaction mixture was stirred at 0° C. for 1 hr and followed by dropwise addition of BnBr (4.01 g, 23.4 mmol). The reaction mixture was further stirred at room temperature overnight. The reaction mixture was quenched with saturated aqueous NH₄Cl (1 mL) at 0° C. The resulting solution was extracted with EA (20 mL×3), dried over Na₂SO₄ and filtered. The filtrate was concentrated, and the crude product was purified by silica gel column (PE/EA=10/1) to give (((20-bromoicosyl)oxy)methyl)-benzene, 51-4, (3.6 g, 66% yield) as an orange-yellow solid. ¹H NMR (400 MHZ, CDCl₃) δ 7.35-7.26 (m, 4H), 7.30-7.25 (m, 1H), 4.50 (s, 2H), 3.46 (t, 2H), 3.41 (t, 2H), 1.85 (m, 2H), 1.61 (m, 2H), 1.41-1.26 (m, 32H).

Step 4. To a solution of ethyl isobutyrate (2.02 g, 17.47 mmol) in THF (50 mL) was added LDA (8.7 mL, 17.47 mmol) at −78° C. under N₂ dropwise. The reaction mixture was stirred at −78° C. for 1 hr. To the mixture was added 51-4 (3.6 g, 13.3 mmol) at −78° C. dropwise. The reaction mixture was warmed up to room temperature and stirred overnight. The reaction mixture was quenched by saturated aqueous NH₄Cl (10 mL) at 0° C. The resulting solution was extracted with EA (50 mL×3), dried over Na₂SO₄ and filtered. The filtrate was concentrated, and the crude product was chromatographed on silica gel (PE/EA=10/1) to give ethyl 22-(benzyloxy)-2,2-dimethyldocosanoate, 51-5, (4.3 g, 97% yield) as a pale-yellow solid. ¹H NMR (400 MHZ, CDCl₃) δ 7.34-7.26 (m, 4H), 7.30-7.25 (m, 1H), 4.50 (s, 2H), 4.11 (q, 2H), 3.46 (t, 2H), 1.63-1.57 (m, 2H), 1.51-1.47 (m, 2H), 1.37-1.22 (m, 37H), 1.15 (s, 6H).

Step 5. To a solution of 51-5 ethyl 22-(benzyloxy)-2,2-dimethyldocosanoate (4.3 g, 7.56 mmol) in THF/MeOH/H₂O (20 mL/20 mL/10 mL) was added NaOH (7.84 g, 196 mmol). The reaction mixture was stirred at 60° C. overnight. The reaction mixture was concentrated under reduced pressure to remove MeOH and THF. The residue was diluted with solvent and poured into water. The aqueous phase was acidified with aqueous HCl and extracted with EA (50 mL×4). The combined organic layers were dried over Na₂SO₄ and concentrated. The crude product was chromatographed on silica gel (PE/EA=1/1) to give (7) 22-(benzyloxy)-2,2-dimethyldocosanoic acid, 51-6, (2.55 g, 71% yield) as a white solid. ¹H NMR (400 MHz, CDCl₃) δ 7.34-7.29 (m, 4H), 7.30-7.25 (m, 1H), 4.50 (s, 2H), 3.46 (t, 2H), 1.61-1.49 (m, 4H), 1.31-1.25 (m, 34H), 1.19 (s, 6H).

Step 6. To a solution of 51-6 (2.55 g, 5.4 mmol) in dry DCM (15 mL) was added (COC1)2 (1.02 g, 8.1 mmol) and DMF (0.5 mL) at 0° C. under N₂ atmosphere. The reaction mixture was stirred at room temperature for 2 hrs. The reaction mixture was concentrated to give crude 22-(benzyloxy)-2,2-dimethyldocosanoyl chloride, 51-7, (5.72 g, crude) as a yellow oil.

Step 7. To a solution of 6-methylpyridin-2-amine (2.5 g, 23.2 mmol) and TEA (3.55 g, 34.8 mmol) in THF (25 mL) was added a solution of 51-7 (5.72 g, 11.6 mmol) in THF (25 mL) dropwise. The reaction mixture was stirred at room temperature overnight under N₂. The reaction mixture was extracted with EA (50 mL×4). The combined organic layers were dried with Na₂SO₄ and concentrated. The crude product was chromatographed on silica gel (PE/EA=10/1) to give 22-(benzyloxy)-2,2-dimethyl-N-(6-methylpyridin-2-yl) docosanamide, 51-8, (6.3 g, 97% yield) as an orange solid. ¹H NMR (400 MHZ, CDCl₃) δ 8.06 (d, J=11.2 Hz, 1H), 7.90 (brs, 1H), 7.58 (t, J=10.4 Hz, 1H), 7.35-7.26 (m, 5H), 6.88 (d, J=10.0 Hz, 1H), 4.50 (s, 2H), 3.46 (t, J=8.8 Hz, 2H), 2.45 (s, 3H), 1.63-1.57 (m, 6H), 1.28 (s, 6H), 1.28-1.24 (m, 32H).

Step 8. To a solution of 51-8 (1.23 mg, 2.2 mmol) in THF (10 mL) was added LiAlH₄ (836 mg, 22 mmol) in portions at room temperature. The mixture was stirred at 60° C. overnight under N₂. The reaction was complete detected by TLC. The reaction mixture was added H₂O (0.8 mL) at 0° C. Then 15% of NaOH aqueous solution (0.8 mL) and water (2.4 mL) were added. The resulting mixture was diluted with THF, dried with Na₂SO₄, filtered, and the filtrate was concentrated to give N-(22-(benzyloxy)-2,2-dimethyldocosyl)-6-methylpyridin-2-amine, 51-9, (1.2 g, crude) as a pale-green oil. ¹H NMR (400 MHZ, CDCl₃) δ 7.36-7.26 (m, 6H), 6.41 (d, J=9.6 Hz, 1H), 6.21 (d, J=11.2 Hz, 1H), 4.50 (s, 2H), 3.46 (t, J=8.8 Hz, 2H), 2.98 (d, J=8.0 Hz, 2H), 2.36 (s, 3H), 1.69-1.57 (m, 6H), 1.43-1.25 (m, 32H), 0.94 (s, 6H).

Step 9. To a solution of 51-9 (1.2 g, 2.1 mmol) and TEA (636.3 mg, 6.3 mmol) in THF (10 mL) was added a solution of 2-fluoro-4-methoxybenzoyl chloride in THF (10 mL). The reaction mixture was stirred at 40° C. for 4 hrs. The reaction mixture was quenched with H₂O (40 mL) and was extracted by EA (50 mL×2). The combined organic layers were dried with Na₂SO₄ and concentrated. The crude product was chromatographed on silica gel (PE/EA=10/1) to give (13)N-(22-(benzyloxy)-2,2-dimethyldocosyl)-2-fluoro-4-methoxy-N-(6-methylpyridin-2-yl)benzamide, 51-10, (1.2 g, 70% yield) as a white solid. ¹H NMR (400 MHz, CDCl₃) δ 7.35-7.31 (m, 4H), 7.29-7.25 (m, 1H), 7.25-7.23 (m, 1H), 7.11 (t, 1H), 6.85-6.83 (d, 1H), 6.60-6.58 (d, 1H), 6.54-6.51 (dd, 1H), 6.40-6.36 (dd, 1H), 4.50 (s, 2H), 4.13 (s, 2H), 3.73 (s, 3H), 3.48-3.46 (t, 2H), 2.47 (s, 3H), 1.62-1.57 (m, 2H), 1.41-0.97 (m, 36H), 0.83 (s, 6H).

Step 10. To a solution of 51-10 (1.2 g, 1.7 mmol) in MeOH (12 mL) was added Pd/C (120 mg) and Pd(OH)₂ (120 mg). The reaction mixture was stirred at room temperature overnight under H₂. The mixture was filtered and the filtrate was concentrated to give 2-fluoro-N-(22-hydroxy-2,2-dimethyldocosyl)-4-methoxy-N-(6-methylpyridin-2-yl)benzamide, 51-11, (930 mg, 89% yield) as a yellow oil. ¹H NMR (400 MHZ, DMSO-d₆) δ: 7.46 (t, J=7.6 Hz, 1H), 7.07 (t, J=8.0 Hz, 1H), 6.97 (d, J=7.2 Hz, 1H), 6.74 (d, J=7.6 Hz, 1H), 6.67-6.62 (m, 2H), 4.30 (br, 1H), 3.98 (s, 2H), 3.70 (s, 3H), 3.36 (t, J=6.4 Hz, 2H), 2.35 (s, 3H), 1.39 (t, J=6.8 Hz, 2H), 1.29-0.87 (m, 36H), 0.77 (m, 6H). MS (ESI) m/z 613.5 [M+1]⁺.

Step 11. To a solution of 51-11 (930 mg, 1.51 mmol) in acetone (10 mL) was added Jones reagent (3.7 mL, 7.55 mmol) at 0° C. dropwise. The reaction mixture was stirred at room temperature for 1 hr. The mixture was quenched by H₂O (30 mL) and extracted by EA (50 mL×3). The combined organic layers were dried, concentrated to give crude product. The crude product was purified by silica gel column (PE/EA=3/1) to give 22-(2-fluoro-4-methoxy-N-(6-methylpyridin-2-yl)benzamido)-21,21-dimethyldocosanoic acid, 51-12, (750 mg, 78% yield) as a yellow oil. ¹H NMR (300 MHz, DMSO-d₆) δ: 11.94 (s, 1H), 7.49 (t, J=7.8 Hz, 1H), 7.10 (t, J=8.1 Hz, 1H), 6.98-6.96 (d, J=7.8 Hz, 1H), 6.74-6.72 (d, J=7.8 Hz, 1H), 6.70-6.66 (m, 2H), 3.98 (s, 2H), 3.70 (s, 3H), 2.34 (s, 3H), 2.18 (t, J=7.2 Hz, 1H), 1.50-1.45 (m, 2H), 1.26-0.99 (m, 34H), 0.77 (m, 6H). MS (ESI) m/z 627.3 [M+1]+.

Step 12. To a solution of 51-12 (550 mg, 0.88 mmol) and Intermediate 53 (622 mg, 1.75 mmol) in DMF (1 mL) was added Cs₂CO₃ (860 mg, 2.64 mmol) and TBA (65 mg, 0.076 mmol). The reaction mixture was stirred at 110° C. for 3 days. The reaction mixture was acidified to pH=5, and extracted by EA (30 mL×3). The combined organic layers were dried, concentrated to give crude product. The crude product was purified by silica gel column (PE/EA/HOAc=10/1/0.001) to give(S)-22-(2-(4-(((4-(3-(tert-butoxy)-1-cyclopropyl-3-oxopropyl)pyridin-2-yl)oxy)methyl) piperidin-1-yl)-4-methoxy-N-(6-methylpyridin-2-yl)benzamido)-21,21-dimethyldocosanoic acid, Intermediate 51, (210 mg, 24% yield) as a yellow oil. ¹H NMR (300 MHz, DMSO-d₆): 11.90 (s, 1H), 8.05-8.03 (d, 1H), 7.31-7.25 (t, 1H), 7.10-7.08 (d, 1H), 6.91-6.89 (m, 1H), 6.85-6.83 (m, 1H), 6.69 (s, 1H), 6.52-6.49 (d, 1H), 6.50-6.49 (m, 1H), 6.39-6.38 (br, 1H), 6.22 (s, 1H), 4.20-4.10 (m, 4H), 3.68 (s, 2H), 2.65-2.61 (m, 2H), 2.36 (s, 3H), 2.36-2.34 (m, 1H), 2.19-2.15 (t, 2H), 1.72 (br, 1H), 1.68-1.61 (m, 2H), 1.50-1.82 (m, 4H), 1.29-1.05 (m, 39H), 1.02-0.85 (m, 4H), 0.80-0.68 (m, 6H), 0.57-0.48 (m, 1H), 0.36-0.23 (m, 2H), 0.18-0.12 (m, 1H). MS (ESI) m/z 484.2 [M/2+1]+.

### Synthesis of Intermediate 52

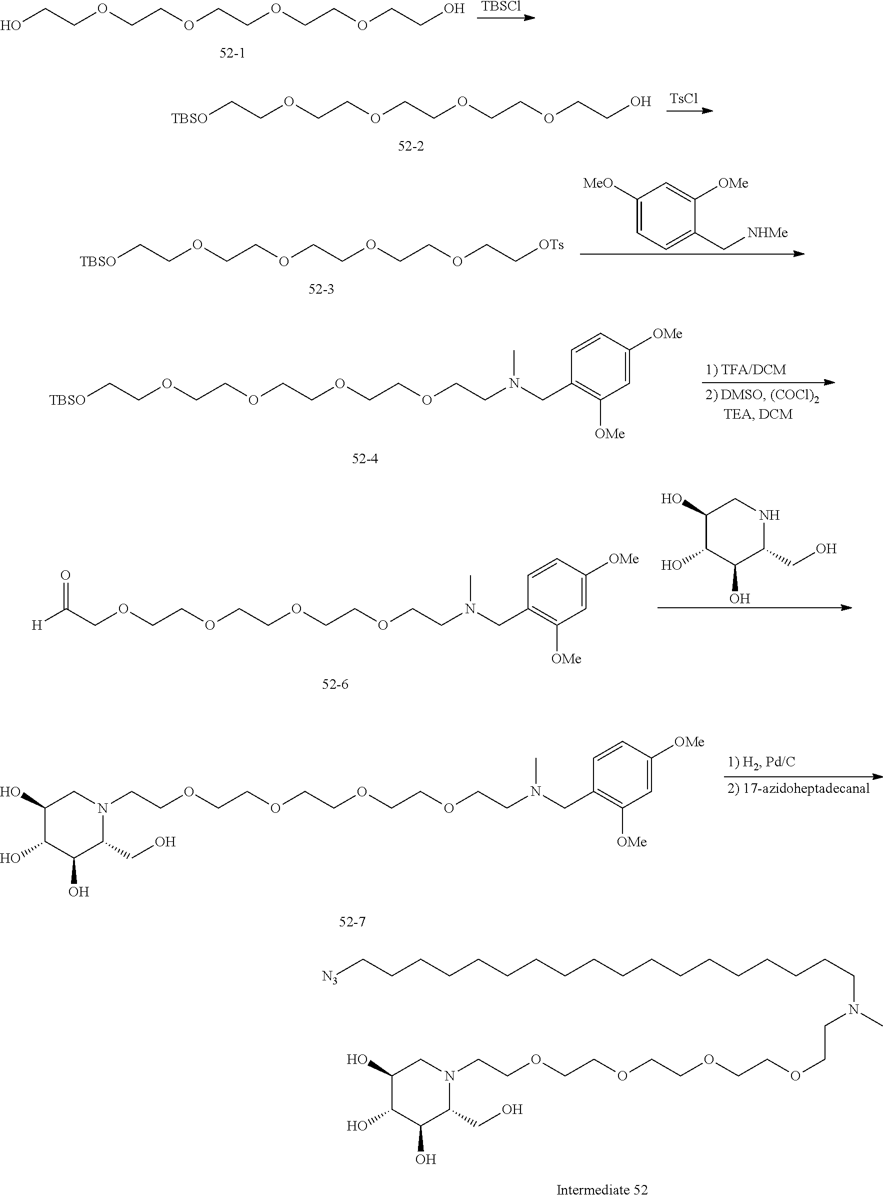

Step 1. To a solution of 3,6,9,12-tetraoxatetradecane-1,14-diol (20 g, 84 mmol) and imidazole (8.5 g, 126 mmol) in DCM (300 mL) cooled to 0° C. was added TBSCl (12.66 g, 84 mmol). The reaction mixture was stirred at room temperature overnight. The mixture was added water (300 mL) and extracted by DCM (500 mL×3) and the organic layer was dried by Na₂SO₄ and concentrated to give a crude produce. The crude produce was purified by column chromatography on silica gel (PE/EA=1/1) to afford 2,2,3,3-tetramethyl-4,7,10,13,16-pentaoxa-3-silaoctadecan-18-ol, 52-2, (15 g, 75% yield) as a yellow oil.

Step 2. To a solution of 52-2 (4 g, 14 mmol) in DCM (50 mL) was added TEA (3.45 g, 34.2 mmol) and TsCl (4.3 g, 22.8 mmol) at 0° C. The reaction mixture was stirred at room temperature overnight. The mixture was extracted by DCM (100 mL×3) and the organic layer was dried by Na₂SO₄ and concentrated to give a crude produce. The crude produce was purified by column chromatography on silica gel (PE/EA=20/1) to afford 2,2,3,3-tetramethyl-4,7,10,13,16-pentaoxa-3-silaoctadecan-18-yl 4-methylbenzenesulfonate, 52-3, (5.2 g, 91.2%) as a yellow oil.

Step 3. To a solution of 52-3 (5 g, 9.8 mmol) in DMF (50 mL) was added K2CO3 (2.7 g, 19.6 mmol) and 1-(2,4-dimethoxyphenyl)-N-methylmethanamine (1.79 g, 9.8 mmol). The resulting reaction mixture was stirred at 90° C. overnight. The mixture was concentrated and the crude produce was purified by column chromatography on silica gel (DCM/MeOH=10/1) to give N-(2,4-dimethoxybenzyl)-N,2,2,3,3-pentamethyl-4,7,10,13,16-pentaoxa-3-silaoctadecan-18-amine, 52-4, (3 g, 60% yield) as a yellow oil. MS (ESI) m/z 516.4 [M+H]⁺

Step 4. To a solution of 52-4 (3 g, 5.8 mmol) in DCM/TFA (15 mL/1 mL) was stirred at room temperature overnight. The mixture was concentrated and was purified by flash to give 1-(2,4-dimethoxyphenyl)-2-methyl-5,8,11,14-tetraoxa-2-azahexadecan-16-ol, 52-5, (1.89 g, 78% yield) as a yellow oil. MS (ESI) m/z 402.3 [M+H]⁺

Step 5. To a solution of oxalyl chloride (1.1 mL, 0.013 mol) in DCM (15 mL) was added DMSO (1.56 g, 0.02 mol) dropwise at −78° C. The resulting reaction mixture was stirred at −78° C. for 1 hr. Then a solution of 52-5 (1.8 g, 0.004 mol) in DCM (5 mL) was added into the above mixture slowly. After stirring at −78° C. for 50 minutes, TEA (2.8 g, 0.028 mol) was added and the reaction mixture was stirred at −78° C. for 1 hr. On completion, the mixture was quenched with water (50 mL) and separated. The aqueous phase was extracted with DCM (3×50 mL). Then the organic phase was combined and washed with brine (50 mL), dried over Na₂SO₄, and concentrated in vacuo to give 1-(2,4-dimethoxyphenyl)-2-methyl-5,8,11,14-tetraoxa-2-azahexadecan-16-al, 52-6, (700 mg, 46.7% yield) as yellow oil. MS (ESI) m/z 401.2 [M+H]⁺

Step 6. To a solution of 52-6 (700 mg, 1.75 mmol) in MeOH (10 mL) was added (2R,3R,4R,5S)-2-(hydroxymethyl) piperidine-3,4,5-triol (571 mg, 3.5 mmol) and NaBH₃CN (220 g, 3.5 mmol) and ZnCl2 (476 mg, 3.5 mmol). The resulting reaction mixture was stirred at 60° C. overnight. The mixture was quenched by water and concentrated to give crude produce. The crude produce was purified by flash to give (2R,3R,4R,5S)-1-(1-(2,4-dimethoxyphenyl)-2-methyl-5,8,11,14-tetraoxa-2-azahexadecan-16-yl)-2-(hydroxymethyl) piperidine-3,4,5-triol, 52-7, (210 mg, 21% yield) as a yellow oil. MS (ESI) m/z 547.3 [M+H]⁺.

Step 7. To a solution of 52-7 in EtOH (10 mL) was added Pd/C (20 mg, 10%). The mixture was stirred at room temperature overnight under H₂. The mixture was filtered and concentrated to give crude produce. The crude produce was purified by prep-HPLC to give (2R,3R,4R,5S)-1-(5,8,11,14-tetraoxa-2-azahexadecan-16-yl)-2-(hydroxymethyl) piperidine-3,4,5-triol, 52-8, (150 mg, 95% yield) as a yellow oil. MS (ESI) m/z 397.3 [M+H]⁺.

Step 8. To a solution of 52-8 (110 mg, 0.28 mmol) and 18-azidooctadecanal (171.78 mg, 0.55 mmol) in MeOH (5 mL) was added NaBH₃CN(52.08 mg, 0.84 mmol) and ZnCl2 (152.3 mg, 1.12 mmol). The resulting reaction mixture was stirred at 60° C. overnight. The mixture was quenched by H₂O and concentrated to give crude produce. The crude produce was purified by prep-HPLC to give (12)(2R,3R,4R,5S)-1-(33-azido-15-methyl-3,6,9,12-tetraoxa-15-azatritriacontyl)-2-(hydroxymethyl) piperidine-3,4,5-triol, Intermediate 52, (40 mg, 21% yield) as a yellow oil. MS (ESI) m/z 690.6 [M+H]⁺.

### Synthesis of Intermediate 53

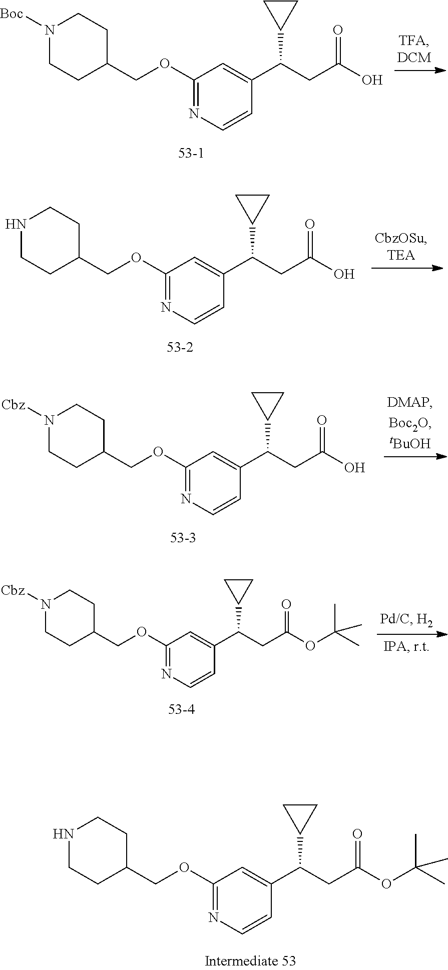 [SMILES: CC(C)(C)OC(=O)N1CCC(COc2cc([C@@H](CC(=O)O)C3CC3)ccn2)CC1 ; O=C(O)C[C@H](c1ccnc(OCC2CCNCC2)c1)C1CC1 ; O=C(O)C[C@H](c1ccnc(OCC2CCN(C(=O)OCc3ccccc3)CC2)c1)C1CC1 ; CC(C)(C)OC(=O)C[C@H](c1ccnc(OCC2CCN(C(=O)OCc3ccccc3)CC2)c1)C1CC1 ; CC(C)(C)OC(=O)C[C@H](c1ccnc(OCC2CCNCC2)c1)C1CC1]

Step 1. To a mixture of(S)-3-(2-((1-(tert-butoxycarbonyl) piperidin-4-yl)methoxy)pyridine-4-yl)-3-cyclopropylpropanoic acid (11 g, 27.2 mmol) in DCM (100 mL) at 0° C. was added TFA (25 mL) dropwise. After addition the resulting mixture was stirred for 1.5 hrs at r. t. Solvent was removed and the residue was coevaporated with toluene twice to give crude(S)-3-cyclopropyl-3-(2-(piperidin-4-ylmethoxy)pyridin-4-yl) propanoic acid, 53-2, (TFA salt, 20 g crude) as a pale-yellow gum. MS (ESI) m/z=305.2 [M+H]⁺.

Step 2. To a mixture of 53-2 (TFA salt, 20 g crude, 0.0272 mmol) and TEA (13.9 g, 0.136 mol) in THF (150 mL) at 0° C. was added CbzOsu (8.13 g, 0.0326 mol) in portions. After addition the resulting suspension was stirred for 12 hrs at r. t. The reaction mixture became clear. Solvent was removed and the residue was diluted with water (100 mL), extracted with EA (60 mL×3), dried and concentrated. The residue was purified by flash chromatography (20% to 35% of EA in PE) to give(S)-3-(2-((1-((benzyloxy) carbonyl) piperidin-4-yl)methoxy)pyridin-4-yl)-3-cyclopropylpropanoic acid, 53-3, (10.2 g, yield: 85%) as a colorless gum. MS (ESI) m/z=439.2 [M+H]⁺. ¹H NMR (400 MHz, DMSO-d₆) δ 12.01 (s, 1H), 8.02-8.01 (d, 1H), 7.39-7.29 (m, 5H), 6.91-6.89 (d, 1H), 6.69 (s, 1H), 5.07 (s, 2H), 4.09-4.00 (m, 2H), 2.90-2.76 (m, 2H), 2.68-2.65 (m, 2H), 2.25-2.21 (m, 1H), 1.98-1.92 (m, 1H), 1.78-1.72 (m, 2H), 1.24-1.12 (m, 2H), 1.04-0.94 (m, 1H), 0.54-0.47 (m, 1H), 0.35-0.23 (m, 2H), 0.18-0.13 (m, 1H).

Step 3. To a mixture of 53-3 (10.2 g, 0.0233 mol) in tBuOH (110 mL) was added Boc₂₀ (10.1 g, 0.0466 mol) and DMAP (850 mg, 6.98 mmol). After addition the resulting mixture was stirred for 2 hrs at 35° C. LCMS indicated the completion of reaction. Solvent was removed and the residue was purified by flash chromatography (10% of EA in PE) to give benzyl(S)-4-(((4-(3-(tert-butoxy)-1-cyclopropyl-3-oxopropyl)pyridin-2-yl)oxy)methyl) piperidine-1-carboxylate, 53-4, (7.5 g, yield: 65%) as a colorless gum. MS (ESI) m/z=439.1 [M+H]⁺.

Step 4. A flask charged with 53-4 (7.5 g, 15.15 mmol) and Pd/C (wet, ˜1.4 g) in isopropyl alcohol (120 mL) was degassed and filled with hydrogen using a balloon. The resulting mixture was then hydrogenated for 1.5 hrs at r. t. LCMS indicated the completion of reaction. The mixture was filtered over celite, and the filter cake was washed with EA/MeOH. The filtrate was combined and concentrated to give tert-butyl(S)-3-cyclopropyl-3-(2-(piperidin-4-ylmethoxy)pyridin-4-yl) propanoate, Intermediate 53, (5.5 g, 100% yield) as a colorless gum. MS (ESI) m/z=361.2 [M+H]⁺. ¹H NMR (400 MHZ, DMSO-d₆) δ 8.02-8.01 (d, 1H), 6.90-6.88 (d, 1H), 6.67 (s, 1H), 4.06-4.04 (d, 2H), 2.96-2.92 (m, 2H), 2.69-2.62 (m, 2H), 2.45-2.41 (m, 2H), 2.25-2.15 (m, 1H), 1.83-1.77 (m, 1H), 1.66-1.63 (m, 2H), 1.26 (s, 9H), 1.17-1.07 (m, 2H), 1.02-0.96 (m, 1H), 0.54-0.48 (m, 1H), 0.37-0.22 (m, 2H), 0.17-0.13 (m, 1H).

### Intermediate 54

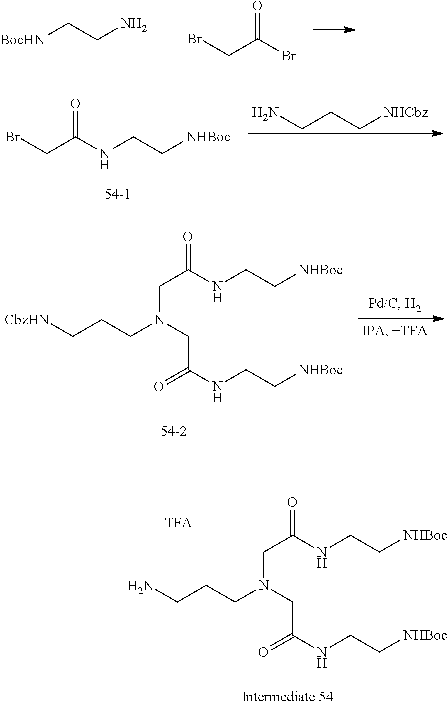 [SMILES: CC(C)(C)OC(=O)NCCN ; O=C(Br)CBr ; CC(C)(C)OC(=O)NCCNC(=O)CBr ; NCCCNC(=O)OCc1ccccc1 ; CC(C)(C)OC(=O)NCCNC(=O)CN(CCCNC(=O)OCc1ccccc1)CC(=O)NCCNC(=O)OC(C)(C)C ; CC(C)(C)OC(=O)NCCNC(=O)CN(CCCN)CC(=O)NCCNC(=O)OC(C)(C)C]

Step 1. To a mixture of tert-butyl(2-aminoethyl) carbamate (20 g, 0.125 mol) and TEA (18.95 g, 0.187 mol) in DCM (200 mL) at 0° C. was added dropwise 2-bromoacetyl bromide (30 g, 0.15 mol) in DCM (30 mL). The mixture was stirred for 12 hrs at r. t., quenched with water (100 mL) and extracted with DCM. The combined organic phase was dried and concentrated. The residue was purified by silica gel column chromatography (PE/EA=5:1 to 2:1) to give tert-butyl(2-(2-bromoacetamido)ethyl) carbamate, 54-1, (9.5 g, yield: 27%) as pale yellow solid. MS (ESI) m/z 181.1 [M+H−100]⁺. ¹H NMR (400 MHZ, DMSO-d₆): 8.26 (s, 1H), 6.80 (s, 1H), 3.83 (s, 2H), 3.12-3.07 (m, 2H), 3.00-2.96 (m, 2H), 1.37 (s, 9H).

Step 2. A mixture of 54-1 (5 g, 17.79 mmol), (3-amino-propyl)-carbamic acid benzyl ester (1.5 g, 6.15 mmol) and DIEA (4.0 g, 30.7 mmol) in MeCN(100 mL) was heated at 70° C. for 16 hrs. The reaction was cooled to r. t. and the product (3-{bis-[(2-tert-butoxycarbonylamino-ethylcarbamoyl)-methyl]-amino}-propyl)-carbamic acid benzyl ester, 54-2, was collected by filtration as pale-yellow solid (2.3 g, yield: 61%). MS (ESI) m/z 609.3 [M+H]⁺. ¹H NMR (400 MHZ, DMSO-d₆): 8.06 (t, J=5.6 Hz, 2H), 7.37-7.28 (m, 5H), 7.23-7.20 (m, 1H), 6.79 (t, J=5.2 Hz, 2H), 4.99 (s, 2H), 3.12-3.09 (m, 4H), 3.03-2.98 (m, 10H), 2.49-2.43 (m, 2H), 1.55-1.51 (m, 2H), 1.36 (s, 18H).

Step 3. A flask charged with 54-2 (500 mg, 0.821 mmol) and Pd/C (wet, ˜100 mg) in isopropyl alcohol (30 mL) was degassed and filled with hydrogen using a balloon. The mixture was then hydrogenated for 2 hrs at 25° C. The mixture was filtered over celite, washed with EA and IPA. The filtrate was combined and concentrated to give crude [2-(4-{(2-amino-ethyl)-[3-(2-tert-butoxycarbonylamino-ethylcarbamoyl)-propyl]-amino}-butyrylamino)-ethyl]-carbamic acid tert-butyl ester, Intermediate 54, (390 mg, yield: ˜100%) as a white gum. MS (ESI) m/z 475.3 [M+H]⁺.

### Intermediate 55

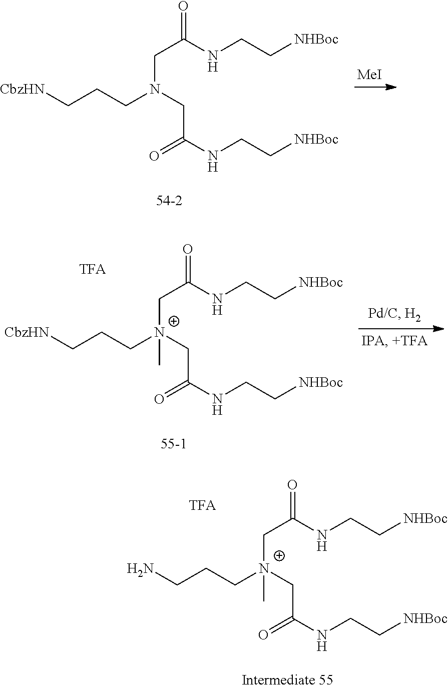 [SMILES: "MeI" ; CC(C)(C)OC(=O)NCCNC(=O)CN(CCCNC(=O)OCc1ccccc1)CC(=O)NCCNC(=O)OC(C)(C)C ; CC(C)(C)OC(=O)NCCNC(=O)C[N+](C)(CCCNC(=O)OCc1ccccc1)CC(=O)NCCNC(=O)OC(C)(C)C ; CC(C)(C)OC(=O)NCCNC(=O)C[N+](C)(CCCN)CC(=O)NCCNC(=O)OC(C)(C)C]

Step 1. A pressure tube charged with 54-2 (600 mg, 0.985 mmol) and Mel (2 mL, 32 mmol) in ACN(7 mL) was sealed and heated to 60° C. for 20 hrs. The reaction mixture was concentrated and the residue was purified by prep-HPLC (TFA method) to give 3-(((benzyloxy) carbonyl)amino)-N,N-bis(2-((2-((tert-butoxycarbonyl)amino)ethyl)amino)-2-oxoethyl)-N-methylpropan-1-aminium, 55-1, (450 mg, yield: 73% yield) as a white gum after freeze drying. MS (ESI) m/z 623.3 [M+]⁺. ¹H NMR (400 MHZ, DMSO-d₆): 8.65 (t, J=5.6 Hz, 2H), 7.41-7.31 (m, 5H), 6.84 (t, J=5.6 Hz, 2H), 5.02 (s, 2H), 4.23 (s, 4H), 3.68-3.63 (m, 2H), 3.29 (s, 3H), 3.13-2.91 (m, 10H), 1.89-1.85 (m, 2H), 1.37 (s, 18H).

Step 2. A flask charged with 55-1 (250 mg, 0.21 mmol) and Pd/C (wet, ˜50 mg) in isopropyl alcohol (20 mL) was degassed and filled with hydrogen using a balloon. The mixture was then hydrogenated for 2 hrs at 30° C. The mixture was filtered over celite, washed with EA and IPA. The filtrate was combined and concentrated to give crude 3-amino-N,N-bis(2-((2-((tert-butoxycarbonyl)amino)ethyl)amino)-2-oxoethyl)-N-methylpropan-1-aminium, Intermediate 55, (190 mg, yield: 97%) as a white gum. MS (ESI) m/z 489.3 [M+] +. ¹H NMR (400 MHZ, DMSO-d₆): 8.75 (t, J=5.6 Hz, 2H), 8.00 (brs, 2H), 6.87-6.82 (m, 2H), 4.29-4.22 (m, 4H), 3.76-3.72 (m, 2H), 3.35 (s, 3H), 3.14-3.11 (m, 4H), 3.04-3.00 (m, 4H), 2.90-2.85 (m, 2H), 2.07-1.99 (m, 2H), 1.37 (s, 18H).

### Intermediate 56

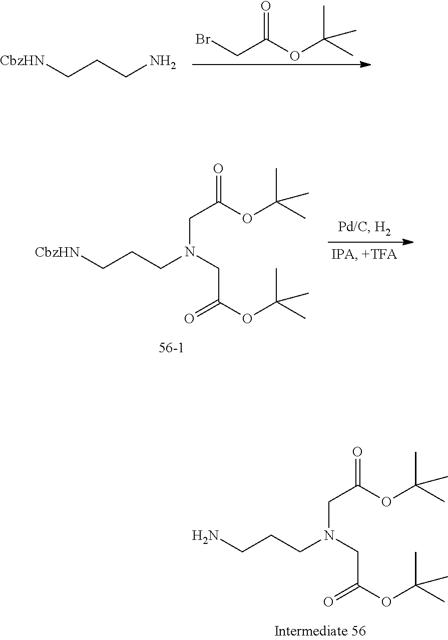

Step 1. To a mixture of (3-amino-propyl)-carbamic acid benzyl ester (HCl salt, 500 mg, 2.409 mmol) and DIEA (1.05 g, 8 mmol) in MeCN(40 mL) at r. t. was added dropwise bromo-acetic acid tert-butyl ester (1.0 g, 5.12 mmol). After addition the resulting mixture was heated at 75° C. for 16 hrs. Solvent was removed and the residue was diluted with EA (20 mL), washed with water and brine and concentrated. The residue was purified by silica gel column chromatography (PE/EA=8:1 to 4:1) to give [(3-benzyloxycarbonylamino-propyl)-tert-butoxycarbonylmethyl-amino]-acetic acid tert-butyl ester, 56-1, (700 mg, yield: 78.4) as a brown gum. MS (ESI) m/z 437.2 [M+H]⁺. ¹H NMR (400 MHZ, DMSO-d₆)7.35-7.29 (m, 5H), 7.17 (t, J=5.6 Hz, 1H), 4.99 (s, 2H), 3.33 (s, 4H), 3.05-3.01 (m, 2H), 2.59 (t, J=7.2 Hz, 2H), 1.53-1.49 (m, 2H), 1.39 (s, 18H).

Step 2. A flask charged with 56-1 (790 mg, 0.821 mmol) and Pd/C (wet, ˜200 mg) in isopropyl alcohol (50 mL) was degassed and filled with hydrogen using a balloon. The mixture was then hydrogenated for 2 hrs at 25° C. The mixture was filtered over celite, washed with EA and IPA. The filtrate was combined and concentrated to give crude [(3-amino-propyl)-tert-butoxycarbonylmethyl-amino]-acetic acid tert-butyl ester, Intermediate 56, (540 mg, yield: 98%) as a pale-yellow gum. MS (ESI) m/z 303.2 [M+H]⁺. ¹H NMR (400 MHZ, DMSO-d₆)3.32 (s, 4H), 2.61 (t, J=6.8 Hz, 2H), 2.54-2.50 (m, 2H), 1.46-1.42 (m, 2H), 1.40 (s, 18H).

### Intermediate 57

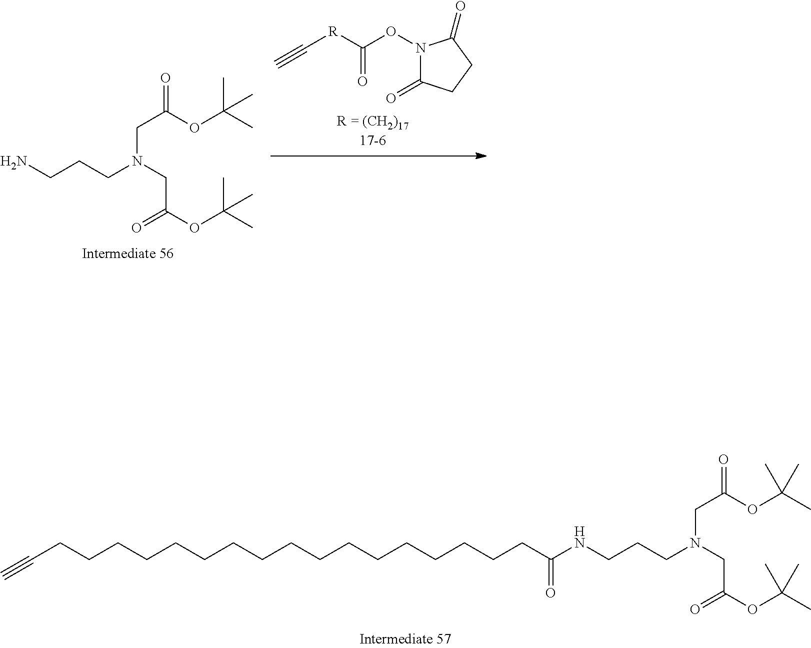

To a mixture of Intermediate 56 (220 mg, 0.726 mmol) and TEA (111 mg, 1.089 mmol) in THF (20 mL) was added dropwise icos-19-ynoic acid 2,5-dioxo-pyrrolidin-1-yl ester, 17-6, (212 mg, 0.727 mmol). After addition the resulting mixture was heated at 45° C. for 16 hrs. Solvent was removed and the residue was diluted with EA (20 mL), washed with water and brine, dried and concentrated to give crude [tert-butoxycarbonylmethyl-(3-icos-19-ynoylamino-propyl)-amino]-acetic acid tert-butyl ester, Intermediate 57, (435 mg, yield ˜100%) as a pale-yellow solid. MS (ESI) m/z 593.4 [M+H]⁺. ¹H NMR (400 MHZ, DMSO-d₆)7.70 (t, J=5.6 Hz, 1H), 3.33 (s, 4H), 3.05-3.02 (m, 2H), 2.71 (t, J=2.8 Hz, 1H), 2.58 (t, J=7.2 Hz, 2H), 2.15-2.12 (m, 2H), 2.03-1.99 (m, 2H), 1.56-1.40 (m, 24H), 1.35-1.23 (m, 26H).

### Intermediate 58

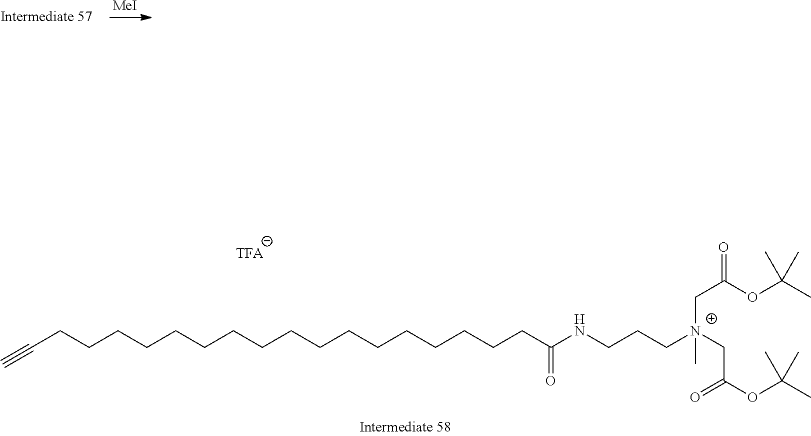 [SMILES: "MeI" ; C#CCCCCCCCCCCCCCCCCCC(=O)NCCC[N+](C)(CC(=O)OC(C)(C)C)CC(=O)OC(C)(C)C]

A pressure tube charged with Intermediate 57 (300 mg, 0.506 mmol) and Mel (1.5 mL, 24 mmol) in ACN(3 mL) was sealed and heated at 60° C. for 20 hrs. The reaction mixture was concentrated and the residue was purified by prep-HPLC (TFA method) to give N,N-bis(2-(tert-butoxy)-2-oxoethyl)-3-(icos-19-ynamido)-N-methylpropan-1-aminium, Intermediate 58, (170 mg, yield: 55% yield) as a white gum after freeze drying. MS (ESI) m/z 607.5 [M+]⁺. ¹H NMR (400 MHZ, DMSO-d₆): 7.89 (t, J=5.6 Hz, 1H), 4.45 (s, 4H), 3.59-3.55 (m, 2H), 3.28 (s, 3H), 3.11-3.06 (m, 2H), 2.71 (t, J=2.8 Hz, 1H), 2.15-2.11 (m, 2H), 2.05-2.01 (m, 2H), 1.86-1.78 (m, 2H), 1.62-1.42 (m, 22H), 1.40-1.25 (m, 26H).

### Intermediate 59

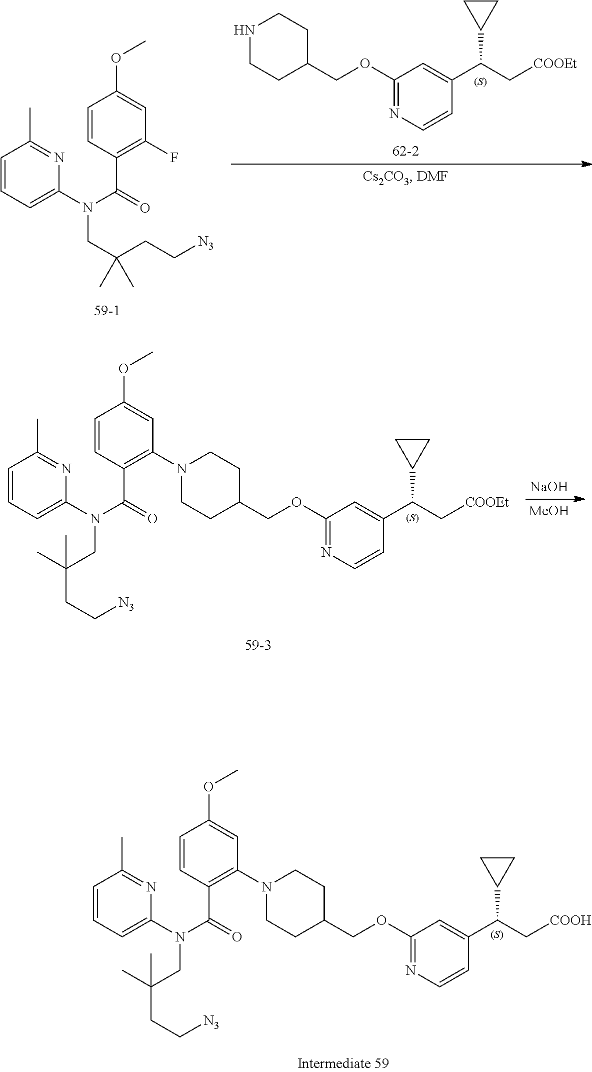

Step 1. Intermediate 59-1 was prepared in the same way as intermediate 30-8. To a solution of 59-1 (1.7 g, 4.42 mmol) in DMF (4 mL) was added(S)-ethyl 3-cyclopropyl-3-(2-(piperidin-4-ylmethoxy)pyridin-4-yl) propanoate (59-2)(2.20 g, 6.62 mmol), Cs₂CO₃ (4.32 g, 13.25 mmol) and Bu₄NI (0.49 g, 1.32 mmol). The mixture was stirred at 110° C. for 72 h. The reaction mixture was quenched with water and extracted with EA. The organic phase was washed with water and brine, dried with Na₂SO₄, filtered, and the filtrate was concentrated. The residue was purified by column chromatography on silica gel (EA/PE=0-10%) to give(S)-ethyl 3-(2-((1-(2-((4-azido-2,2-dimethylbutyl)(6-methylpyridin-2-yl) carbamoyl)-5-methoxyphenyl) piperidin-4-yl)methoxy)pyridin-4-yl)-3-cyclopropylpropanoate, intermediate 59-3, (0.6 g, yield: 19.5%) as a yellow oil. MS (ESI) m/z 698.4 [M+H]⁺.

Step 2. To a solution of 59-3 (600 mg, 0.86 mmol) in MeOH (5 mL) was added NaOH aq. (1.29 mL, 2.58 mmol, 2M), and the mixture was stirred at 45° C. for 4 h. The reaction mixture was diluted with water and adjusted to pH=5 with HCl (2N). The resulting mixture was extracted with EA. The organic phase was washed with water and brine, dried with Na₂SO₄, filtered, and the filtrate was concentrated to give(S)-3-(2-((1-(2-((4-azido-2,2-dimethylbutyl)(6-methylpyridin-2-yl) carbamoyl)-5-methoxyphenyl) piperidin-4-yl)methoxy)pyridin-4-yl)-3-cyclopropylpropanoic acid, Intermediate 59, (520 mg, yield: 90.3%) as a yellow gum. MS (ESI) m/z 670.2 [M+H]⁺. ¹H NMR (400 MHZ, DMSO-d₆): 8.05-8.04 (d, 1H), 7.31-7.27 (t, 1H), 7.16-7.14 (d, 1H), 6.93-6.92 (d, 1H), 6.88-6.86 (d, 1H), 6.73 (s, 1H), 6.55-6.52 (dd, 1H), 6.45 (br, 1H), 6.23-6.22 (d, 1H), 4.14-4.13 (m, 4H), 3.69 (s, 3H), 3.29 (br, 2H), 2.70-2.68 (d, 2H), 2.33 (s, 3H), 2.27-2.20 (m, 1H), 1.78-1.74 (br, 1H), 1.74-1.69 (m, 2H), 1.45-1.31 (m, 4H), 1.02-0.99 (m, 1H), 0.78 (s, 6H), 0.53-0.48 (m, 1H), 0.37-0.23 (m, 2H), 0.19-0.16 (m, 1H).

### Intermediate 60

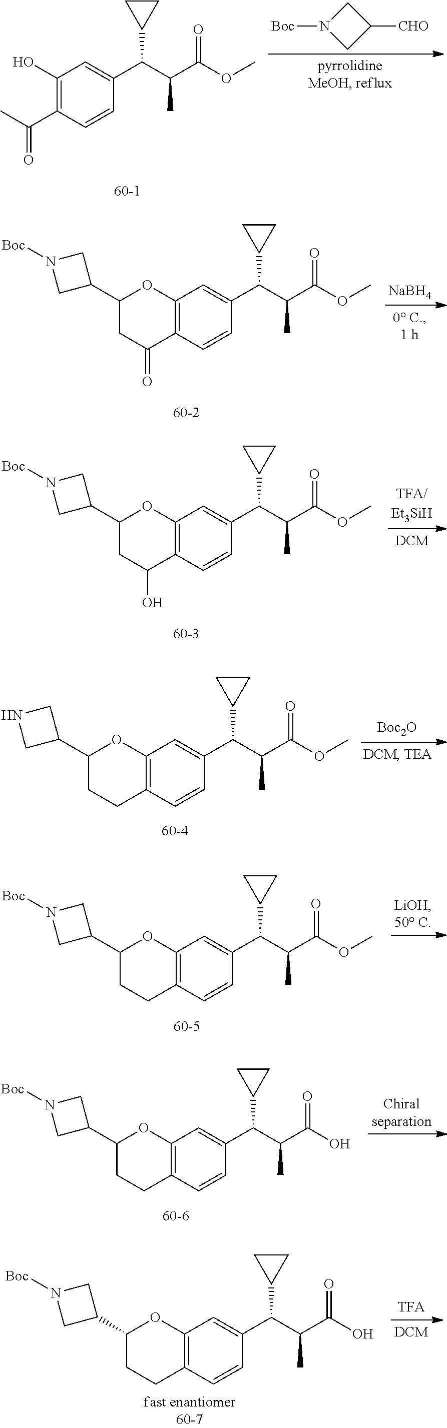

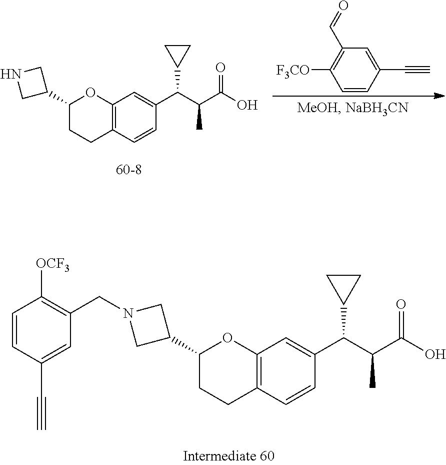 [SMILES: C[C@H](C(=O)O)[C@H](c1ccc2c(c1)O[C@@H](C1CNC1)CC2)C1CC1 ; C#Cc1ccc(OC(F)(F)F)c(C=O)c1 ; C#Cc1ccc(OC(F)(F)F)c(CN2CC([C@H]3CCc4ccc([C@H](C5CC5)[C@H](C)C(=O)O)cc4O3)C2)c1]

Step 1. To a solution of (2S,3R)-methyl 3-(4-acetyl-3-hydroxyphenyl)-3-cyclopropyl-2-methylpropanoate (60-1)(2.5 g, 9.8 mmol), tert-butyl 3-formylazetidine-1-carboxylate (2.2 g, 11.76 mmol) in MeOH (30 mL) was added pyrrolidine (1.0 g, 14.7 mmol). The resulting reaction mixture was stirred at 70° C. for 10 hrs. The mixture was concentrated and the residue was purified by column chromatography on silica gel (PE/EA=4/1) to afford tert-butyl 3-(7-((1R,2S)-1-cyclopropyl-3-methoxy-2-methyl-3-oxopropyl)-4-oxochroman-2-yl) azetidine-1-carboxylate, 60-2, (3.6 g, 69% yield) as a as a yellow oil. MS (ESI) m/z 467.2 [M+23]⁺.

Step 2. To a mixture of 60-2 (3.6 g, 8 mmol) in MeOH (50 mL) at 0° C. was added NaBH₄ (470 mg, 12 mmol) in portions. The resulting reaction mixture was stirred for 1 h, allowing the temperature rising to room temperature. The mixture was quenched aq. NH₄Cl (˜2 mL) and concentrated. The residue was purified by column chromatography on silica gel (PE/EA=3/1) to afford tert-butyl 3-(7-((1R,2S)-1-cyclopropyl-3-methoxy-2-methyl-3-oxopropyl)-4-hydroxy \chroman-2-yl) azetidine-1-carboxylate, 60-3, (3.4 g, 95.5% yield) as a yellow gum. MS (ESI) m/z 446.3. [M+H]⁺.

Step 3. To a solution of 60-3 (3.4 g, 7.4 mol) in DCM (30 mL) was added TFA (7 mL). The resulting reaction mixture was stirred at room temperature for 1 hr. Then the mixture was added Et₃SiH (7 mL) and stirred at room temperature for 12 hrs. Solvent was removed and the residue was basified with aq. NaHCO₃until pH reached 8 to 9, extracted with EA (80 mL×3), dried and concentrated to give crude (2S,3R)-methyl 3-(2-(azetidin-3-yl) chroman-7-yl)-3-cyclopropyl-2-methylpropanoate, 60-4, (3 g, crude) as a yellow gum. MS (ESI) m/z 330.2 [M+H]⁺.

Step 4. To a mixture of 60-4 (3 g, crude, 9.1 mmol) and TEA (2.76 g, 27.3 mmol) in DCM (50 mL) at 0° C. was added Boc₂O (2.3 g, 10.92 mmol) dropwise. The resulting reaction mixture was stirred at room temperature overnight. The mixture was concentrated and the residue was purified by column chromatography on silica gel (PE/EA=2/1) to afford tert-butyl 3-(7-((1R,2S)-1-cyclopropyl-3-methoxy-2-methyl-3-oxopropyl) chroman-2-yl) azetidine-1-carboxylate, 60-5, (1.7 g, 42% yield) as a yellow gum. MS (ESI) m/z 452.3 [M+23]⁺.

Step 5. To a mixture of 60-5 (1.2 g, 2.8 mmol) in THF/MeOH/H2O (12 mL/6 mL/6 mL) was added LiOH·H₂O (1.11 g, 28 mmol). The resulting reaction mixture was stirred and heated at 55° C. for 16 hrs. Volatiles were removed and the aqueous layer was acidified with 1M HCl until pH reached 3 to 4, extracted with EA (50 mL×4), dried and concentrated. The residue was purified by RP flash chromatography to give (2S,3R)-3-(2-(1-(tert-butoxycarbonyl) azetidin-3-yl) chroman-7-yl)-3-cyclopropyl-2-methylpropanoic acid, 60-6, (850 mg, 68% yield) as white solid. MS (ESI) m/z 316.2 [M-100]⁺.

Step 6. Chiral separation. Product 60-7 (the faster eluting isomer) was obtained by chiral separation on SFC-IG colukn eluting with 80% CO₂ and 20% IPA containing 0.2% of TFA.

Step 7. A mixture of 60-7 (592 mg, 1.42 mmol) in DCM (5 mL) and TFA (1 mL) was stirred at room temperature for 2 hrs. The mixture was then concentrated to give (2S,3R)-3-((R)-2-(azetidin-3-yl) chroman-7-yl)-3-cyclopropyl-2-methylpropanoic acid, 60-8, (449 mg, crude) as a yellow oil, which was directly used in the next step.

Step 8. A mixture of 60-8 (100 mg, 0.317 mmol) and 5-ethynyl-2-(trifluoromethoxy)benzaldehyde (203 mg, 0.95 mmol) in MeOH (5 mL) was stirred for 1 hr at r. t. Then NaBH₃CN(60 mg, 0.95 mmol) was added. The resulting reaction mixture was stirred at room temperature for 16 hrs. The reaction mixture was quenched by the addition of saturated aqueous NH₄Cl and concentrated. The residue was purified by prep-HPLC (TFA method) to give (2S,3R)-3-cyclopropyl-3-((R)-2-(1-(5-ethynyl-2-(trifluoromethoxy)benzyl) azetidin-3-yl) chroman-7-yl)-2-methylpropanoic acid, Intermediate 60, (12 mg, 7.5% yield over 2 steps) as a white solid, which was lyophilized and exchanged as HCl salt. ¹H NMR (400 MHZ, DMSO-d₆): 7.79 (s, 1H), 7.71-7.69 (d, 1H), 7.53-7.51 (d, 1H), 7.01-6.99 (d, 1H), 6.71-6.69 (d, 1H), 6.64 (s, 1H), 4.52-4.49 (m, 2H), 4.41 (s, 1H), 4.25 (br, 2H), 4.18-4.16 (m, 3H), 3.18-3.08 (m, 1H), 2.88-2.63 (m, 3H), 2.00-1.83 (m, 2H), 1.62-1.43 (m, 1H), 1.10-1.02 (m, 1H), 0.83-0.81 (d, 3H), 0.57-0.50 (m, 1H), 0.28-0.22 (m, 2H), −0.05−−0.10 (m, 1H). MS (ESI) m/z 514.3 [M+H]⁺.

### Intermediate 61

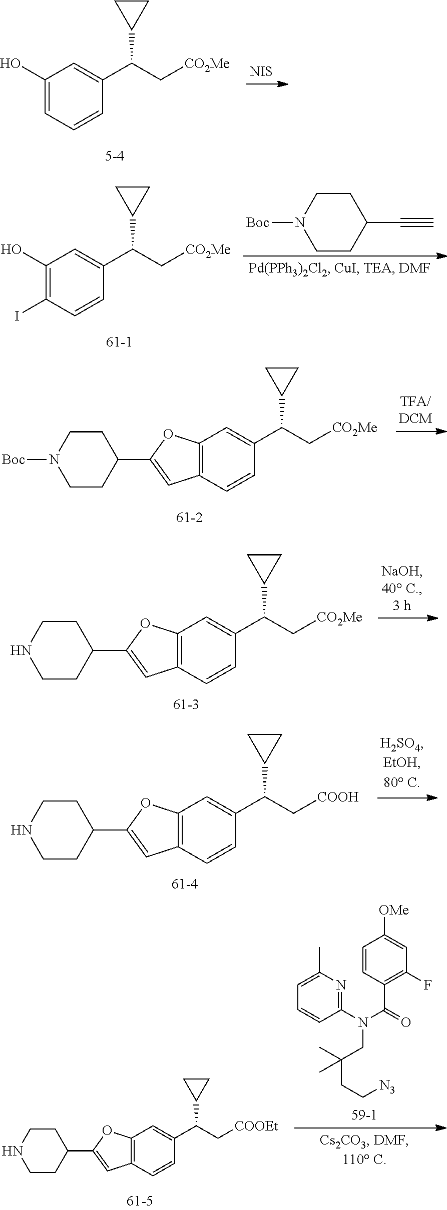

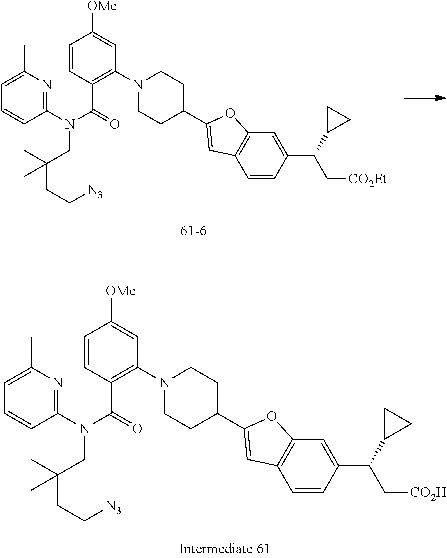 [SMILES: CCOC(=O)C[C@H](c1ccc2cc(C3CCN(c4cc(OC)ccc4C(=O)N(CC(C)(C)CCN=[N+]=[N-])c4cccc(C)n4)CC3)oc2c1)C1CC1 ; COc1ccc(C(=O)N(CC(C)(C)CCN=[N+]=[N-])c2cccc(C)n2)c(N2CCC(c3cc4ccc([C@@H](CC(=O)O)C5CC5)cc4o3)CC2)c1]

Step 1. To a mixture of 3-cyclopropyl-3-(3-hydroxy-phenyl)-propionic acid methyl ester, 5-4, (2.02 g, 9.182 mmol) in DCM at 0° C. was added NIS (2.16 g, 9.64 mmol) in portions. The resulting mixture was stirred for 12 h, allowing the temperature slowly warm to r. t. Solvent was removed and the residue was purified by flash chromatography (10% EA in PE) to give 3-cyclopropyl-3-(3-hydroxy-4-iodo-phenyl)-propionic acid methyl ester, 61-1, (2.4 g, yield: 75%) as yellow solid. MS (ESI) m/z 347.1 [M+H]⁺. ¹H NMR (400 MHZ, DMSO-d₆) δ 10.15 (s, 1H), 7.54 (d, J=8.0 Hz, 1H), 6.75 (s, 1H), 6.53-6.51 (m, 1H), 3.51 (s, 3H), 2.74-2.62 (m, 2H), 2.20-2.15 (m, 1H), 0.98-0.92 (m, 1H), 0.53-0.46 (m, 1H), 0.35-0.29 (m, 1H), 0.22-0.17 (m, 1H), 0.11-0.05 (m, 1H).

Step 2. A flask charged with 61-1 (1.0 g, 2.88 mmol), 4-ethynyl-piperidine-1-carboxylic acid tert-butyl ester (903 mg, 4.32 mmol), Pd(PPh₃)₂C12 (202 mg, 0.288 mmol), CuI (55 mg, 0.288 mmol) and TEA (1.45 g, 14.4 mmol) in DMF was degassed and filled with N2. The resulting mixture was then heated at 75° C. for 16 hrs. Diluted with water and extracted with EA 3 times. The organic phase was combined, dried and concentrated. The residue was purified by flash chromatography (15% EA in PE) to give 4-[6-(1-cyclopropyl-2-methoxycarbonyl-ethyl)-benzofuran-2-yl]-piperidine-1-carboxylic acid tert-butyl ester, 61-2, (600 mg, yield: 48%) as a yellow gum. MS (ESI) m/z 372.1 [M-55]⁺. ¹H NMR (400 MHz, DMSO-d₆) δ 7.43-7.41 (m, 1H), 7.09 (d, J=9.2 Hz, 1H), 6.55 (s, 1H), 4.02-3.96 (m, 1H), 3.48 (s, 3H), 2.81-2.73 (m, 2H), 2.38-2.33 (m, 1H), 2.02-1.96 (m, 2H), 1.55-1.45 (m, 2H), 1.40 (s, 9H), 1.09-1.01 (m, 1H), 0.53-0.47 (m, 1H), 0.32-0.19 (m, 2H), 0.14-0.09 (m, 1H).

Step 3. To a mixture of 61-2 (1.0 g, 2.34 mmol) in DCM (12 mL) was added TFA (3 mL) dropwise. The resulting mixture was stirred for 2 hr at room temperature. Solvent was removed and the residue was basified with aq. NaHCO₃until pH reached 9 to 10 and extracted with EA 3 times. The organic phase was combined, dried and concentrated to give methyl(S)-3-cyclopropyl-3-(2-(piperidin-4-yl)benzofuran-6-yl), 61-3, (650 mg, yield: 86%) as a brown gum. MS (ESI) m/z 328.2 [M+H]⁺.

Step 4. A mixture of 61-3 (560 mg, 1.715 mmol) and NaOH (137 mg, 3.425 mmol) in MeOH/THF/H2O (5 mL/5 mL/5 mL) was stirred for 3 h at 40° C. The reaction mixture was acidified with 1 M HCl to the pH 2, dried and concentrated to give crude 3-cyclopropyl-3-(2-piperidin-4-yl-benzofuran-6-yl)-propionic acid, 61-4, (550 mg crude) which was directly used in the next step. MS (ESI) m/z 314.2 [M+H]⁺.

Step 5. To a mixture of 61-4 (550 mg, 1.715 mmol) in EtOH (20 mL) was added conc. H₂SO₄ (0.5 mL) dropwise. After addition the resulting mixture was heated to 80° C. for 4h. Solvent was removed and the residue was basified with aq. NaHCO₃until the pH reached 8, extracted with EA (30 mL×4), dried and concentrated. The residue was purified by flash chromatography (8% of DCM in MeOH) to give 3-cyclopropyl-3-(2-piperidin-4-yl-benzofuran-6-yl)-propionic acid ethyl ester, 61-5, (300 mg, yield: 51% over 2 steps) as pale-yellow solid. MS (ESI) m/z 342.2 [M+H]⁺.

Step 6. A vial charged with 59-1 (160 mg, 0.418 mmol), 61-5 (200 mg, 0.585 mmol), Cs2CO3 (410 mg, 1.254 mmol) and TBAI (20 mg, cat.) in dry DMF (1.2 mL) was heated under N₂ atmosphere at 110° C. for 40 hrs. The mixture was diluted with water and extracted with EA 3 times. The organic phase was combined, dried and concentrated. The residue was by prep-TLC (PE:EA=2:1) to give 3-[2-(1-{2-[(4-azido-2,2-dimethyl-butyl)-(6-methyl-pyridin-2-yl)-carbamoyl]-5-methoxy-phenyl}-piperidin-4-yl)-benzofuran-6-yl]-3-cyclopropyl-propionic acid ethyl ester, 61-6, (60 mg, yield: 19.4%) as a yellow solid. MS (ESI) m/z 707.3 [M+H]⁺.

Step 7. A mixture of 61-6 (60 mg, 0.085 mmol) and LiOH·H₂O (10 mg, 0.25 mmol) in water/MeOH/THF (2 mL/2 mL/2 mL) was stirred at 40° C. for 2 h. Excess solvent was removed and the residue was acidified with HCl (1M) to pH 3 ˜ 4, and extracted with EA, dried and concentrated. The residue was purified by prep-TLC (PE:EA=1:1) to give 3-[2-(1-{2-[(4-azido-2,2-dimethyl-butyl)-(6-methyl-pyridin-2-yl)-carbamoyl]-5-methoxy-phenyl}-piperidin-4-yl)-benzofuran-6-yl]-3-cyclopropyl-propionic acid, Intermediate 61, as a yellow solid. MS (ESI) m/z 679.2 [M+H]⁺. ¹H NMR (400 MHZ, DMSO-d₆) δ 11.96 (brs, 1H), 7.48-7.46 (d, 1H), 7.41 (s, 1H), 7.32-7.28 (m, 1H), 7.19-7.17 (d, 1H), 7.13-7.11 (d, 1H), 6.89-6.87 (d, 1H), 6.58-6.55 (m, 2H), 6.44 (br, 1H), 6.26 (s, 1H), 4.24-4.00 (m, 2H), 3.70 (s, 3H), 2.80-2.65 (m, 4H), 2.40-2.32 (m, 1H), 2.37 (s, 3H), 2.00-1.90 (m, 5H), 1.63-1.52 (m, 1H), 1.45-1.26 (m, 1H), 1.10-1.03 (m, 1H), 0.84-0.71 (m, 6H), 0.54-0.49 (m, 1H), 0.32-0.24 (m, 2H), 0.15-0.10 (m, 1H).

### Intermediate 62

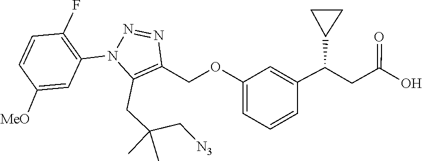 [SMILES: COc1ccc(F)c(-n2nnc(COc3cccc([C@@H](CC(=O)O)C4CC4)c3)c2CC(C)(C)CN=[N+]=[N-])c1]

MS (ESI) m/z 523.1 [M+H]⁺. ¹H NMR (400 MHZ, DMSO-d₆): δ 7.53-7.37 (t, 1H), 7.37-7.35 (m, 1H), 7.26-7.22 (m, 2H), 6.96 (s, 1H), 6.96-6.93 (m, 1H), 6.89-6.87 (m, 1H), 5.17 (s, 2H), 3.81 (s, 3H), 3.13 (s, 2H), 2.77 (s, 2H), 2.70-2.60 (m, 2H), 2.32-2.07 (m, 1H), 1.04-0.99 (m, 1H), 0.67 (s, 6H), 0.52-0.48 (m, 1H), 0.34-0.24 (m, 2H), 0.15-0.12 (m, 1H).

### Intermediate 63

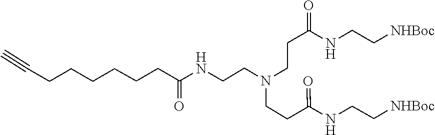 [SMILES: C#CCCCCCCC(=O)NCCN(CCC(=O)NCCNC(=O)OC(C)(C)C)CCC(=O)NCCNC(=O)OC(C)(C)C]

MS (ESI) m/z 625.4 [M+H]⁺. ¹H NMR (400 MHZ, DMSO-d₆): δ 7.90 (t, NH, 2H), 7.59 (t, NH, 1H), 6.76 (t, NH, 2H), 3.10-3.03 (m, 6H), 2.98-2.95 (m, 4H), 2.72-2.71 (t, 1H), 2.67-2.62 (t, 4H), 2.41-2.39 (m, 2H), 2.19-2.10 (m, 6H), 2.02-1.99 (t, 2H), 1.50-1.25 (m, 8H), 1.37 (s, 18H).

### Intermediate 64

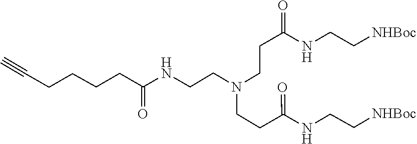 [SMILES: C#CCCCCC(=O)NCCN(CCC(=O)NCCNC(=O)OC(C)(C)C)CCC(=O)NCCNC(=O)OC(C)(C)C]

MS (ESI) m/z 597.4 [M+H]⁺. ¹H NMR (400 MHZ, DMSO-d₆): δ 7.88 (t, NH, 2H), 7.61 (t, NH, 1H), 6.76 (t, NH, 2H), 3.10-3.03 (m, 6H), 2.98-2.95 (m, 4H), 2.72-2.71 (t, 1H), 2.72-2.64 (t, 4H), 2.44-2.40 (m, 2H), 2.19-2.12 (m, 6H), 2.08-2.04 (t, 2H), 1.58-1.53 (m, 2H), 1.45-1.31 ((m, 2H), 1.37 (s, 18H).

### Intermediate 65

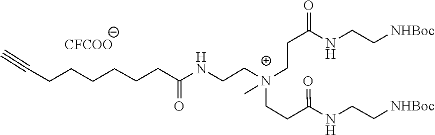 [SMILES: C#CCCCCCCC(=O)NCC[N+](C)(CCC(=O)NCCNC(=O)OC(C)(C)C)CCC(=O)NCCNC(=O)OC(C)(C)C ; "CFCOO"]

MS (ESI) m/z 639.4 [M]⁺. ¹H NMR (400 MHZ, DMSO-d₆): δ 8.17 (t, NH, 2H), 8.10 (t, NH, 1H), 6.82 (t, NH, 2H), 3.57-3.53 (m, 4H), 3,46-3.40 (m, 2H), 3.31-3.28 (m, 2H), 3.10-3.07 (m, 4H), 3.02-2.98 (m, 7H), 2.74-2.72 (t, 1H), 2.68-2.60 (m, 4H), 2.16-2.08 (m, 4H), 1.58-1.30 (m, 6H), 1.37 (s, 18H), 1.27-1.22 ((m, 2H).

### Intermediate 66

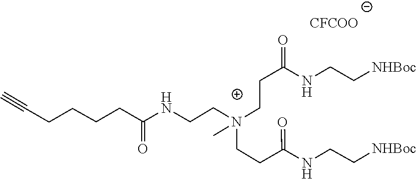

MS (ESI) m/z 611.4 [M]⁺. ¹H NMR (400 MHz, DMSO-d₆): δ 8.15 (t, NH, 2H), 8.12 (t, NH, 1H), 6.82 (t, NH, 2H), 3.57-3.53 (m, 4H), 3,46-3.42 (m, 2H), 3.31-3.28 (m, 2H), 3.10-3.07 (m, 4H), 3.02-2.98 (m, 7H), 2.76-2.75 (t, 1H), 2.66-2.60 (m, 4H), 2.17-2.10 (m, 4H), 1.60-1.56 (m, 2H), 1.46-1.37 ((m, 2H), 1.37 (s, 18H).

### Intermediate 67

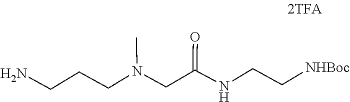 [SMILES: CN(CCCN)CC(=O)NCCNC(=O)OC(C)(C)C]

MS (ESI) m/z 289.2 [M+H]⁺. ¹H NMR (400 MHZ, DMSO-d₆): δ 7.85 (t, NH, 1H), 6.83 (t, NH, 1H), 3.14-3.10 (m, 2H), 3.02-2.97 (m, 2H), 2.86 (s, 2H), 2.58-2.55 (m, 2H), 2.38-2.34 (m, 2H), 2.16 (s, 3H), 1.50-1.46 (m, 2H), 1.38 (s, 9H).

### Intermediate 68

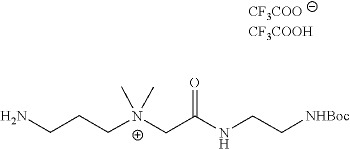 [SMILES: CC(C)(C)OC(=O)NCCNC(=O)C[N+](C)(C)CCCN]

MS (ESI) m/z 303.2 [M]⁺. ¹H NMR (400 MHZ, DMSO-d₆): δ 8.67 (t, NH, 1H), 6.88 (t, NH, 1H), 4.01 (s, 2H), 3.54-3.51 (m, 2H), 3.18 (s, 6H), 317-3.13 (m, 2H), 3.06-3.01 (m, 2H), 2.86-2.82 (t, 2H), 1.98-1.94 (m, 2H), 1.38 (s, 9H).

### Intermediate 69

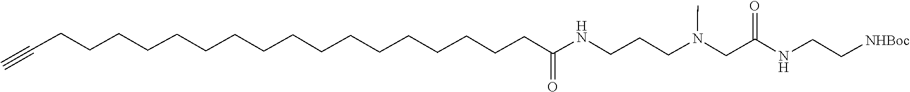 [SMILES: C#CCCCCCCCCCCCCCCCCCC(=O)NCCCN(C)CC(=O)NCCNC(=O)OC(C)(C)C]

MS (ESI) m/z 579.5 [M+H]⁺. ¹H NMR (400 MHZ, DMSO-d₆): δ 3.13-3.07 (m, 4H), 3.01-2.98 (m, 2H), 2.85 (s, 2H), 2.73-2.71 (t, 1H), 2.35-2.31 (m, 2H), 2.14 (s, 3H), 2.14-2.11 (m, 2H), 2.05-2.01 (m, 2H), 1.54-1.30 (m, 6H), 1.38 (s, 9H), 1.31-1.29 (br, 26H).

### Intermediate 70

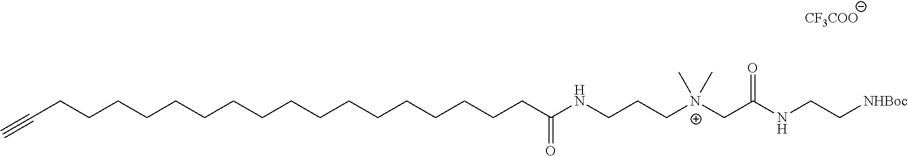 [SMILES: "CF3COO" ; C#CCCCCCCCCCCCCCCCCCC(=O)NCCC[N+](C)(C)CC(=O)NCCNC(=O)OC(C)(C)C]

MS (ESI) m/z 593.5 [M]⁺. ¹H NMR (400 MHZ, DMSO-d₆): δ 8.58 (t, NH, 1H), 7.90 (t, NH, 1H), 6.88 (t, NH, 1H), 3.97 (s, 2H), 3.15 (s, 6H), 3.15-3.01 (m, 4H), 2.51-2.50 (t, 1H), 2.15-2.11 (m, 2H), 2.06-2.03 (m, 2H), 1.84-1.79 (m, 2H), 1.49-1.27 (m, 4H), 1.38 (s, 9), 1.31-1.29 (m, 26H).

### Intermediate 71

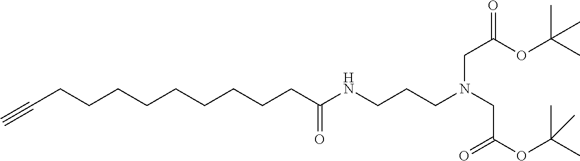 [SMILES: C#CCCCCCCCCCC(=O)NCCCN(CC(=O)OC(C)(C)C)CC(=O)OC(C)(C)C]

MS (ESI) m/z 481.3 [M+H]⁺. ¹H NMR (400 MHZ, DMSO-d₆): δ 7.71 (t, NH, 1H), 3.33 (s, 4H), 3.07-3.03 (m, 2H), 2.72-2.71 (t, 1H), 2.60-2.57 (t, 2H), 2.15-2.11 (m, 2H), 2.04-2.00 (m, 2H), 1.49-1.44 (m, 6H), 1.41 (s, 18H), 1.25-1.17 (br, 10H).

### Intermediate 72

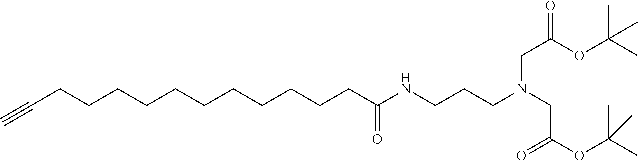 [SMILES: C#CCCCCCCCCCCCC(=O)NCCCN(CC(=O)OC(C)(C)C)CC(=O)OC(C)(C)C]

MS (ESI) m/z 509.3 [M+H]⁺. ¹H NMR (400 MHZ, DMSO-d₆): δ 7.71 (t, NH, 1H), 3.50 (s, 4H), 3.07-3.03 (m, 2H), 2.84-2.80 (br, 2H), 2.73-2.71 (t, 1H), 2.15-2.11 (m, 2H), 2.04-2.00 (m, 2H), 1.49-1.44 (m, 6H), 1.41 (s, 18H), 1.25-1.17 (br, 14H).

### Intermediate 73

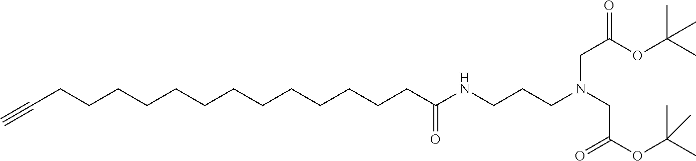 [SMILES: C#CCCCCCCCCCCCCCC(=O)NCCCN(CC(=O)OC(C)(C)C)CC(=O)OC(C)(C)C]

MS (ESI) m/z 537.4 [M+H]⁺. ¹H NMR (400 MHZ, DMSO-d₆): δ 7.72 (t, NH, 1H), 3.32 (s, 4H), 3.06-3.04 (m, 2H), 2.72-2.71 (t, 1H), 2.59-2.56 (m, 2H), 2.17-2.13 (m, 2H), 2.04-1.97 (m, 2H), 1.47-1.30 (m, 6H), 1.41 (s, 18H), 1.25-1.17 (br, 18H).

### Intermediate 74

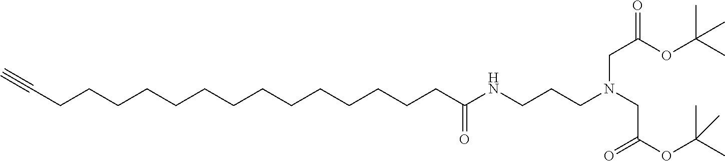 [SMILES: C#CCCCCCCCCCCCCCCC(=O)NCCCN(CC(=O)OC(C)(C)C)CC(=O)OC(C)(C)C]

MS (ESI) m/z 551.4 [M]⁺. ¹H NMR (400 MHZ, DMSO-d₆): δ 7.72 (t, NH, 1H), 3.56 (s, 4H), 3.07-3.03 (m, 2H), 2.73-2.71 (t, 1H), 2.59-2.56 (m, 2H), 2.15-2.11 (m, 2H), 2.04-2.00 (m, 2H), 1.49-1.44 (m, 6H), 1.41 (s, 18H), 1.25-1.17 (br, 20H).

### Intermediate 75

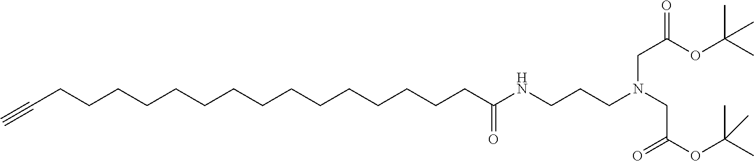 [SMILES: C#CCCCCCCCCCCCCCCCC(=O)NCCCN(CC(=O)OC(C)(C)C)CC(=O)OC(C)(C)C]

MS (ESI) m/z 565.4 [M]⁺. ¹H NMR (400 MHZ, DMSO-d₆): δ 7.76 (t, NH, 1H), 3.56 (s, 4H), 3.08-3.03 (m, 2H), 2.73-2.71 (t, 1H), 2.74-3.71 (m, 2H), 2.15-2.11 (m, 2H), 2.04-2.00 (m, 2H), 1.55-1.50 (m, 2H), 1.49-1.44 (m, 4H), 1.41 (s, 18H), 1.25-1.17 (br, 22H).

### Intermediate 76

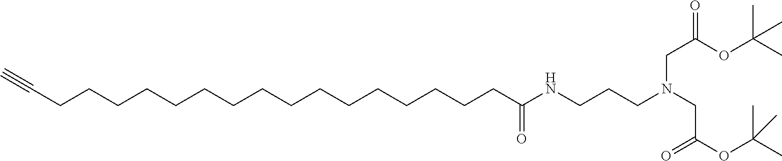 [SMILES: C#CCCCCCCCCCCCCCCCCC(=O)NCCCN(CC(=O)OC(C)(C)C)CC(=O)OC(C)(C)C]

MS (ESI) m/z 579.5 [M+H]⁺. ¹H NMR (400 MHZ, CDCl₃): δ 7.55 (t, NH, 1H), 3.41-3.38 (m, 2H), (m, 2H), 3.36 (s, 4H), 2.73-2.70 (t, 2H), 2.21-2.15 (m, 4H), 1.94-1.92 (t, 1H), 1.62-1.49 (m, 6H), 1.44 (s, 18H), 1.25-1.17 (br, 24H).

### Intermediate 77

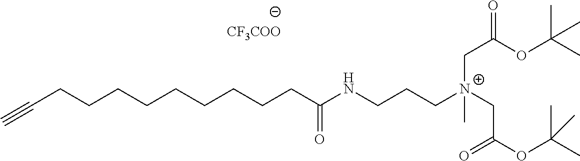 [SMILES: C#CCCCCCCCCCC(=O)NCCC[N+](C)(CC(=O)OC(C)(C)C)CC(=O)OC(C)(C)C ; "CF3COO"]

MS (ESI) m/z 495.3 [M+H]⁺. ¹H NMR (400 MHZ, DMSO-d₆): δ 7.88 (t, NH, 1H), 4.45 (s, 4H), 3.30 (s, 3H), 3.11-3.06 (m, 2H), 2.73-2.72 (t, 1H), 2.16-2.12 (m, 2H), 2.05-2.02 (m, 2H), 1.86-1.80 (m, 4H), 1.83 (s, 18H), 1.30-1.20 (br, 10H).

### Intermediate 78

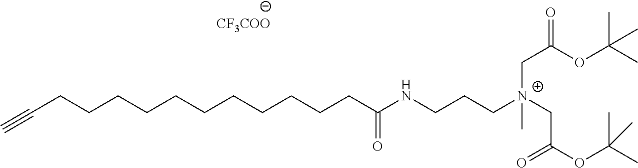 [SMILES: C#CCCCCCCCCCCCC(=O)NCCC[N+](C)(CC(=O)OC(C)(C)C)CC(=O)OC(C)(C)C ; "CF3COO"]

MS (ESI) m/z 523.4 [M]⁺. ¹H NMR (400 MHZ, DMSO-d₆): δ 7.88 (t, NH, 1H), 4.43 (s, 4H), 3.59-3.55 (m, 2H), 3.28 (s, 3H), 3.11-3.06 (m, 2H), 2.73-2.71 (t, 1H), 2.16-1.96 (m, 2H), 1.83-1.80 (m, 2H), 1.50-1.38 (m, 4H), 1.45 (s, 18H), 1.31-1.20 (m, 14H).

### Intermediate 79

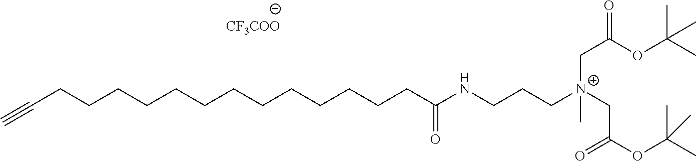 [SMILES: C#CCCCCCCCCCCCCCC(=O)NCCC[N+](C)(CC(=O)OC(C)(C)C)CC(=O)OC(C)(C)C ; "CF3COO"]

MS (ESI) m/z 551.4 [M]⁺. ¹H NMR (400 MHz, DMSO-d₆): δ 7.89 (t, NH, 1H), 4.45 (s, 4H), 3.59-3.55 (m, 2H), 3.29 (s, 3H), 3.12-3.05 (m, 2H), 2.74-2.71 (t, 1H), 2.17-2.13 (m, 2H), 2.07-1.99 (m, 2H), 1.83-1.78 (m, 2H), 1.50-1.40 (m, 4H), 1.45 (s, 18H), 1.31-1.20 (m, 18H).

### Intermediate 80

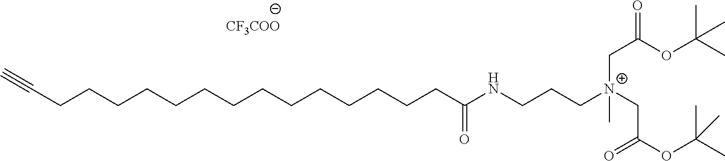 [SMILES: C#CCCCCCCCCCCCCCCC(=O)NCCC[N+](C)(CC(=O)OC(C)(C)C)CC(=O)OC(C)(C)C ; "CF3COO"]

MS (ESI) m/z 565.4 [M]⁺. ¹H NMR (400 MHZ, DMSO-d₆): δ 7.89 (t, NH, 1H), 4.45 (s, 4H), 3.56-3.53 (m, 2H), 3.29 (s, 3H), 3.11-3.05 (m, 2H), 2.73-2.71 (t, 1H), 2.15-2.11 (m, 2H), 2.05-2.02 (m, 2H), 1.86-1.78 (m, 2H), 1.50-1.40 (m, 4H), 1.45 (s, 18H), 1.31-1.20 (m, 20H).

### Intermediate 81

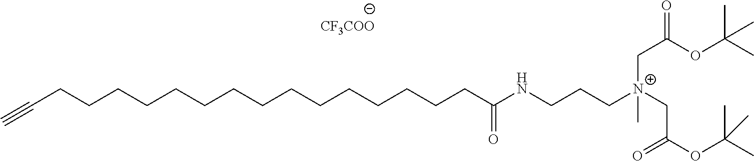 [SMILES: C#CCCCCCCCCCCCCCCCC(=O)NCCC[N+](C)(CC(=O)OC(C)(C)C)CC(=O)OC(C)(C)C ; "CF3COO"]

MS (ESI) m/z 579.5 [M]⁺. ¹H NMR (400 MHZ, DMSO-d₆): δ 7.89 (t, NH, 1H), 4.45 (s, 4H), 3.59-3.55 (m, 2H), 3.28 (s, 3H), 3.11-3.06 (m, 2H), 2.73-2.72 (t, 1H), 2.16-2.11 (m, 2H), 2.05-2.00 (m, 2H), 1.84-1.80 (m, 2H), 1.50-1.40 (m, 4H), 1.45 (s, 18H), 1.31-1.20 (m, 22H).

### Intermediate 82

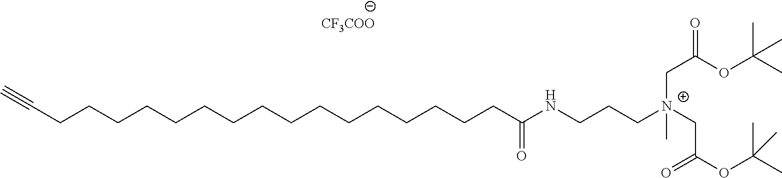 [SMILES: C#CCCCCCCCCCCCCCCCCC(=O)NCCC[N+](C)(CC(=O)OC(C)(C)C)CC(=O)OC(C)(C)C ; "CF3COO"]

MS (ESI) m/z 593.5 [M]⁺. ¹H NMR (400 MHZ, DMSO-d₆): δ 7.90 (t, NH, 1H), 4.45 (s, 4H), 3.59-3.55 (m, 2H), 3.28 (s, 3H), 3.11-3.06 (m, 2H), 2.73-2.72 (t, 1H), 2.15-2.11 (m, 2H), 2.05-2.00 (m, 2H), 1.84-1.80 (m, 2H), 1.50-1.40 (m, 4H), 1.45 (s, 18H), 1.31-1.20 (m, 24H).

### Intermediate 83

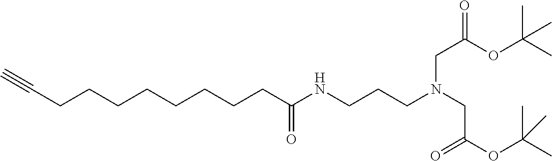 [SMILES: C#CCCCCCCCCC(=O)NCCCN(CC(=O)OC(C)(C)C)CC(=O)OC(C)(C)C]

MS (ESI) m/z 467.4 [M+H]⁺. ¹H NMR (400 MHZ, DMSO-d₆): δ 7.72 (t, NH, 1H), 3.34 (s, 4H), 3.08-3.03 (m, 2H), 2.72-2.71 (t, 1H), 2.61-2.57 (t, 2H), 2.16-2.12 (m, 2H), 2.04-2.01 (m, 2H), 1.49-1.43 (m, 6H), 1.41 (s, 18H), 1.35-1.20 (br, 8H).

### Intermediate 84

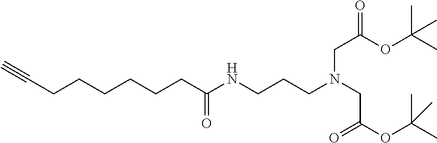 [SMILES: C#CCCCCCCC(=O)NCCCN(CC(=O)OC(C)(C)C)CC(=O)OC(C)(C)C]

MS (ESI) m/z 439.3 [M+H]⁺. ¹H NMR (400 MHZ, DMSO-d₆): δ 7.76 (t, NH, 1H), 3.34 (s, 4H), 3.08-3.03 (m, 2H), 2.72-2.71 (t, 1H), 2.61-2.57 (t, 2H), 2.15-2.11 (m, 2H), 2.05-2.01 (m, 2H), 1.58-1.38 (m, 6H), 1.41 (s, 18H), 1.35-1.20 (br, 4H).

### Intermediate 85

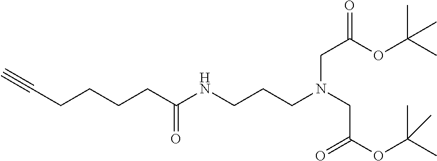 [SMILES: C#CCCCCC(=O)NCCCN(CC(=O)OC(C)(C)C)CC(=O)OC(C)(C)C]

MS (ESI) m/z 411.3 [M+H]⁺. ¹H NMR (400 MHZ, DMSO-d₆): δ 7.84 (t, NH, 1H), 3.34 (s, 4H), 3.09-3.04 (m, 2H), 2.75-2.73 (t, 1H), 2.61-2.57 (t, 2H), 2.15-2.13 (m, 2H), 2.08-2.04 (m, 2H), 1.70-1.55 (m, 6H), 1.41 (s, 18H).

### Intermediate 86

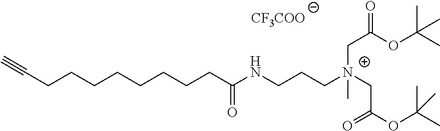 [SMILES: C#CCCCCCCCCC(=O)NCCC[N+](C)(CC(=O)OC(C)(C)C)CC(=O)OC(C)(C)C ; "CF3COO"]

MS (ESI) m/z 481.4 [M]⁺. ¹H NMR (400 MHz, DMSO-d₆): δ 7.88 (t, NH, 1H), 4.46 (s, 4H), 3.60-3.52 (m, 2H), 3.29 (s, 3H), 3.11-3.06 (m, 2H), 2.74 (t, 1H), 2.14-2.11 (m, 2H), 2.06-1.99 (m, 2H), 1.84-1.80 (m, 2H), 1.56-1.38 (m, 4H), 1.48 (s, 18H), 1.37-1.20 (m, 8H).

### Intermediate 87

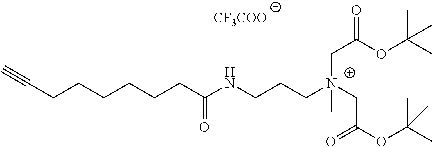 [SMILES: C#CCCCCCCC(=O)NCCC[N+](C)(CC(=O)OC(C)(C)C)CC(=O)OC(C)(C)C ; "CF3COO"]

MS (ESI) m/z 453.3 [M]⁺. ¹H NMR (400 MHZ, DMSO-d₆): δ 7.92 (t, NH, 1H), 4.46 (s, 4H), 3.60-3.52 (m, 2H), 3.29 (s, 3H), 3.11-3.06 (m, 2H), 2.73-3.72 (t, 1H), 2.15-2.12 (m, 2H), 2.07-2.02 (m, 2H), 1.84-1.80 (m, 2H), 1.56-1.38 (m, 4H), 1.48 (s, 18H), 1.37-1.20 (m, 4H).

### Intermediate 88

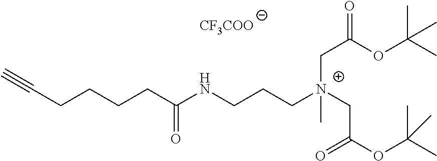 [SMILES: C#CCCCCC(=O)NCCC[N+](C)(CC(=O)OC(C)(C)C)CC(=O)OC(C)(C)C ; "CF3COO"]

MS (ESI) m/z 425.3 [M]⁺. ¹H NMR (400 MHZ, DMSO-d₆): δ 7.99 (t, NH, 1H), 4.49 (s, 4H), 3.65-3.56 (m, 2H), 3.32 (s, 3H), 3.14-3.10 (m, 2H), 2.78-3.77 (t, 1H), 2.20-2.15 (m, 2H), 2.12-2.06 (m, 2H), 1.88-1.84 (m, 2H), 1.60-1.40 (m, 4H), 1.48 (s, 18H).

### Intermediate 89

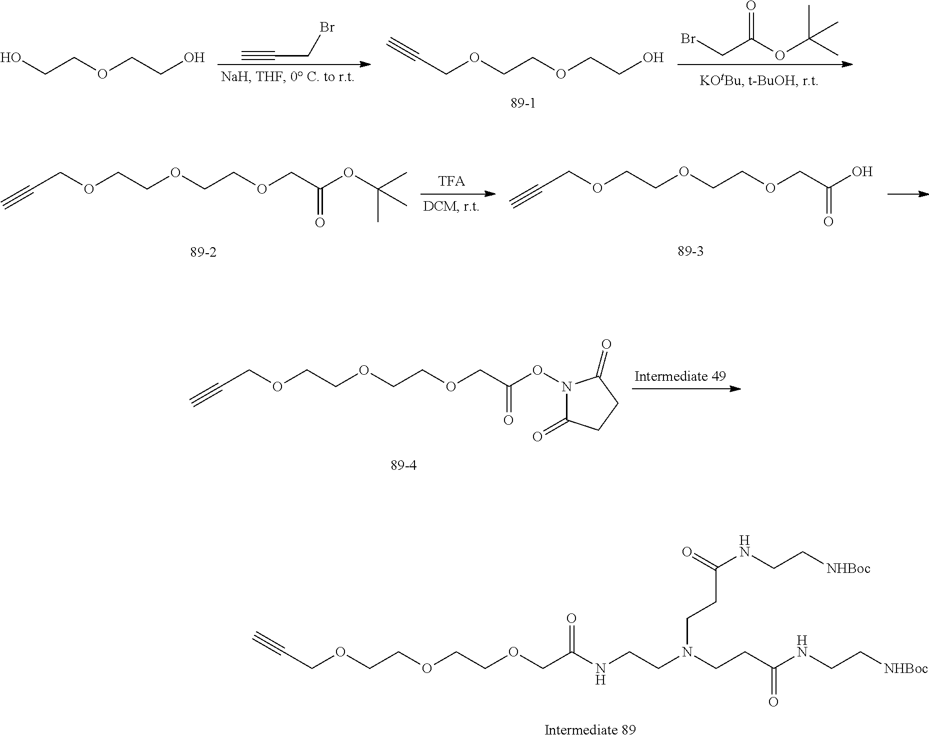

Step 1. To a solution of 2,2′-oxydiethanol (16.5 g, 155.7 mmol) in THF (150 mL) was added NaH (4.48 g, 311.4 mmol) in portions at 0° C. The reaction mixture was stirred at 0° C. for 2 hours, followed by addition of 3-bromoprop-1-yne (18.5 g, 155.7 mmol) dropwise. The reaction mixture was stirred at room temperature overnight under N₂. The reaction mixture was quenched by the addition of saturated aqueous NH₄Cl at 0° C. The resulting solution was extracted with DCM (20 mL×3), dried over Na₂SO₄ and filtered. The filtrate was concentrated, and the crude product was chromatographed on silica gel (Petroleum ether/EtOAc 4:1-2:1->0:1->Dichloromethane/Methanol 10:1) to give 2-(2-(prop-2-yn-1-yloxy) ethoxy) ethanol, 89-1, (8.67 g, yield: 39%) as an orange liquid. ¹H NMR (400 MHZ, CDCl₃) δ 4.21 (d, J=3.2 Hz, 2H), 3.74-3.71 (m, 6H), 3.63-3.60 (m, 2H), 2.45 (t, J=3.2 Hz, 1H), 2.34 (br, 1H).

Step 2. To a solution of 89-1 (8.67 g, 60.2 mmol) in t-BuOH (80 mL) was added t-BuOK (6742 mg, 60.2 mmol) in portions at room temperature. The reaction mixture was stirred at room temperature for 30 minutes, and followed by the addition of tert-butyl 2-bromoacetate (11.74 g, 60.2 mmol). The reaction mixture was stirred at room temperature overnight under N₂. The solvent was removed, and the residue was extracted with DCM (20 mL×3), dried over Na₂SO₄ and filtered. The filtrate was concentrated, and the crude product was chromatographed on silica gel (Petroleum ether/EtOAc 10:1->4:1-0:1) to give tert-butyl 2-(2-(2-(prop-2-yn-1-yloxy) ethoxy) ethoxy)acetate, 89-2, (9.32 g, yield: 60%) as a pale-yellow liquid. ¹H NMR (400 MHZ, CDCl₃) δ 4.20 (d, J=2.8 Hz, 2H), 4.02 (s, 2H), 3.73-3.70 (m, 8H), 2.42 (t, J=2.8 Hz, 1H), 1.47 (s, 9H).

Step 3. To a solution of 89-2 (11.3 g, 44.0 mmol) in DCM (30 mL) was added trifluoroacetic acid (30 mL) dropwise. After addition, the resulting mixture was stirred at room temperature overnight under N₂. The reaction mixture was concentrated under reduced pressure to remove DCM and TFA to give crude 2-(2-(2-(prop-2-yn-1-yloxy) ethoxy) ethoxy) acetic acid, 89-3, (8.9 g, crude) as a purple liquid. MS (ESI) m/z 201.1 [M−H]⁻.

Step 4. To a solution of crude 89-3 (8.9 g, 44.0 mmol) in DCM (40 mL) was added 1-hydroxy-pyrrolidine-2,5-dione (10.12 g, 88.0 mmol) at 0° C. The reaction mixture was stirred at 0° C. for 30 minutes, followed by the addition of EDCI (16.9 g, 88.0 mmol) in portions at 0° C. The reaction mixture was stirred at room temperature overnight under N₂. The reaction mixture was quenched with water (20 mL) and extracted with DCM (30 mL×3). The combined organic layers were washed with brine (20 mL×2) and water (20 mL), dried over Na₂SO₄ and concentrated. The residue was chromatographed on silica gel (Petroleum ether/EtOAc 2:1->1:1->0:1->Dichloromethane/Methanol 10:1) to give 2,5-dioxopyrrolidin-1-yl 2-(2-(2-(prop-2-yn-1-yloxy) ethoxy) ethoxy)acetate, 89-4, (12659 mg, yield: 96%) as an orange liquid. ¹H NMR (400 MHZ, CDCl₃) δ 4.52 (s, 2H), 4.20 (d, J=1.2 Hz, 2H), 3.81-3.79 (m, 2H), 3.72-3.69 (m, 6H), 2.88-2.84 (m, 4H), 2.43 (t, J=2.4 Hz, 1H),

Step 5. To a mixture of Intermediate 49 (3.23 g, 6.6 mmol) and TEA (1.576 g, 9.9 mmol) in THF (40 mL) was added 89-4 (2.18 g, 7.3 mmol). After addition, the resulting mixture was stirred at 40° C. overnight under N₂. The reaction mixture was quenched with water (20 mL) and extracted with EA (20 mL×3), dried over Na₂SO₄ and concentrated. The residue was chromatographed on silica gel (Petroleum ether/EtOAc 0:1->Dichloromethane/Methanol 20:1->10:1) and purified by preparative HPLC (NH₄HCO₃ method) to give Intermediate 89 as a pale-yellow gum. MS (ESI) m/z 673.4 [M+H]⁺. ¹H NMR (400 MHZ, DMSO-d₆): δ=7.87 (s, 2H), 7.53 (t, J=7.6 Hz, 1H), 6.76 (s, 2H), 4.14 (d, J=2.0 Hz, 2H), 3.86 (s, 2H), 3.56-3.49 (m, 8H), 3.43-3.41 (t, 1H), 3.19-3.15 (m, 2H), 3.08-3.04 (m, 4H), 2.98-2.94 (m, 4H), 2.65 (t, J=8.6 Hz, 4H), 2.46-2.42 (m, 2H), 2.17 (t, J=8.6 Hz, 4H), 1.37 (s, 18H). MS (ESI) m/z 709.5 [M+H]⁺.

### Intermediate 90

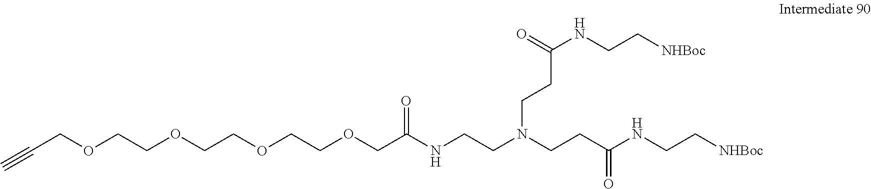 [SMILES: C#CCOCCOCCOCCOCC(=O)NCCN(CCC(=O)NCCNC(=O)OC(C)(C)C)CCC(=O)NCCNC(=O)OC(C)(C)C]

Starting from ethylene glycol trimer, Intermediate 90 was prepared in the same way as Intermediate 89. MS (ESI) m/z 717.5 [M+H]⁺. ¹H NMR (400 MHZ, DMSO-d₆): δ 7.87 (t, J=6.8 Hz, 2H), 7.53 (t, J=7.4 Hz, 1H), 6.76 (t, J=6.8 Hz, 2H), 4.13 (d, J=3.2 Hz, 2H), 3.86 (s, 2H), 3.56-3.52 (m, 12H), 3.41 (t, J=3.2 Hz, 1H), 3.16-3.12 (m, 2H), 3.06-3.02 (m, 4H), 2.98-2.94 (m, 4H), 2.65 (t, J=9.0 Hz, 4H), 2.45 (t, J=8.8 Hz, 2H), 2.17 (t, J=9.2 Hz, 4H), 1.37 (s, 18H).

### Intermediate 91

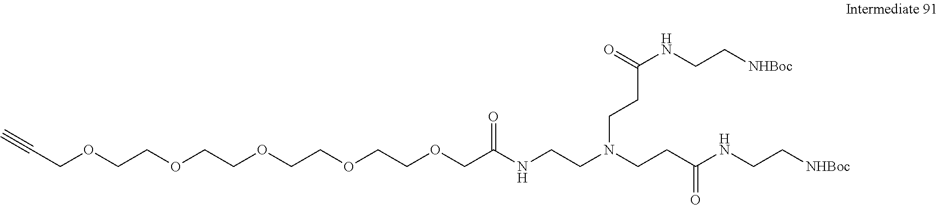 [SMILES: C#CCOCCOCCOCCOCCOCC(=O)NCCN(CCC(=O)NCCNC(=O)OC(C)(C)C)CCC(=O)NCCNC(=O)OC(C)(C)C]

Starting from ethylene glylcol trimer, Intermediate 91 was prepared in the same way as Intermediate 89. MS (ESI) m/z 761.5 [M+H]⁺. ¹H NMR (400 MHZ, DMSO-d₆): δ 7.88 (t, J=6.0 Hz, 2H), 7.54 (t, J=7.2 Hz, 1H), 6.77 (t, J=6.4 Hz, 2H), 4.15 (d, J=3.2 Hz, 2H), 3.86 (s, 2H), 3.57-3.52 (m, 16H), 3.42 (t, J=3.2 Hz, 1H), 3.19-3.13 (m, 2H), 3.07-3.03 (m, 4H), 2.98-2.90 (m, 4H), 2.67-2.62 (m, 4H), 2.46-2.42 (m, 2H), 2.19-2.15 (m, 4H), 1.38 (s, 18H).

### Intermediate 92

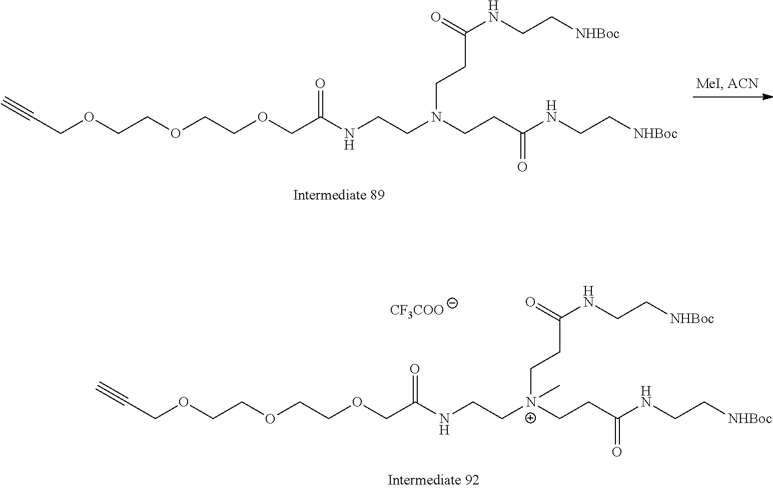 [SMILES: C#CCOCCOCCOCC(=O)NCCN(CCC(=O)NCCNC(=O)OC(C)(C)C)CCC(=O)NCCNC(=O)OC(C)(C)C ; C#CCOCCOCCOCC(=O)NCC[N+](C)(CCC(=O)NCCNC(=O)OC(C)(C)C)CCC(=O)NCCNC(=O)OC(C)(C)C ; "CF3COO"]

A pressure tube (20 mL) charged with Intermediate 89 (983 mg, 1.5 mmol) and Mel (1.5 mL, 24 mmol) in MeCN(6 mL) was sealed and heated at 60° C. overnight. Solvent was removed and the residue (in MeOH) was purified by prep-HPLC (C8 column, TFA method) to give N,N-bis(3-((2-((tert-butoxycarbonyl)amino)ethyl)amino)-3-oxopropyl)-N-methyl-4-oxo-6,9,12-trioxa-3-azapentadec-14-yn-1-aminium trifluoroacetate, Intermediate 92, (591 mg, yield: 59%) as a pale-yellow gum. MS (ESI) m/z 687.5 [M+H]⁺. ¹H NMR (400 MHZ, DMSO-d₆): δ 8.23 (t, J=7.2 Hz, 2H), 8.08 (t, J=7.2 Hz, 1H), 6.83 (t, J=7.0 Hz, 2H), 4.14 (d, J=3.2 Hz, 2H), 3.92 (m, J=5.2 Hz, 2H), 3.57-3.50 (m, 12H), 3.46-3.32 (m, 5H), 3.13-2.89 (m, 11H), 2.66-2.55 (m, 4H), 1.36 (s, 18H).

### Intermediate 93

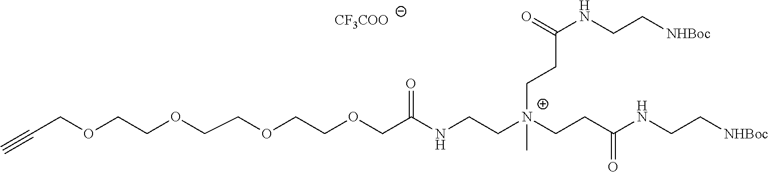 [SMILES: C#CCOCCOCCOCCOCC(=O)NCC[N+](C)(CCC(=O)NCCNC(=O)OC(C)(C)C)CCC(=O)NCCNC(=O)OC(C)(C)C ; "CF3COO"]

Intermediate 93 was prepared from Intermediate 90 in the same way as Intermediate 92. MS (ESI) m/z 731.5 [M+H]⁺. ¹H NMR (400 MHZ, DMSO-d₆): δ 8.19 (t, J=6.2 Hz, 2H), 8.08 (t, J=6.6 Hz, 1H), 6.83 (t, J=4.8 Hz, 2H), 4.14 (d, J=3.2 Hz, 2H), 3.91 (m, J=8.4 Hz, 2H), 3.60-3.50 (m, 16H), 3.45-3.30 (m, 5H), 3.27-2.97 (m, 11H), 2.73-2.61 (m, 4H), 1.37 (s, 18H).

### Intermediate 94

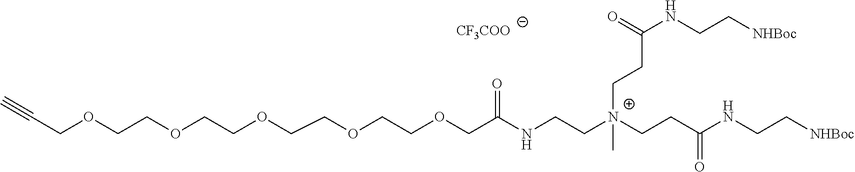 [SMILES: C#CCOCCOCCOCCOCCOCC(=O)NCC[N+](C)(CCC(=O)NCCNC(=O)OC(C)(C)C)CCC(=O)NCCNC(=O)OC(C)(C)C ; "CF3COO"]

Intermediate 94 was prepared from Intermediate 91 in the same way as Intermediate 92. MS (ESI) m/z 775.9 [M+H]⁺. ¹H NMR (400 MHZ, DMSO-d₆): δ 8.19 (t, J=6.2 Hz, 2H), 8.08 (t, J=6.6 Hz, 1H), 6.83 (t, J=4.8 Hz, 2H), 4.14 (d, J=3.2 Hz, 2H), 3.91 (m, J=8.4 Hz, 2H), 3.60-3.50 (m, 20H), 3.45-3.30 (m, 5H), 3.27-2.97 (m, 11H), 2.73-2.61 (m, 4H), 1.37 (s, 18H).

### Intermediate 95

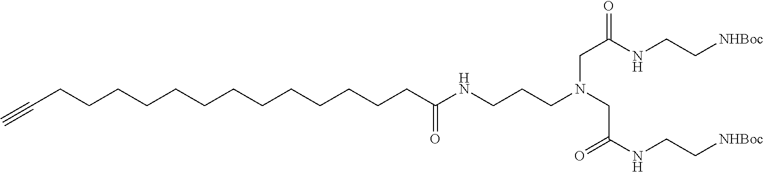 [SMILES: C#CCCCCCCCCCCCCCC(=O)NCCCN(CC(=O)NCCNC(=O)OC(C)(C)C)CC(=O)NCCNC(=O)OC(C)(C)C]

MS (ESI) m/z 709.5 [M+H]⁺. ¹H NMR (400 MHZ, DMSO-d₆): δ 8.07 (t, NH, 2H), 7.73 (t, NH, 1H), 6.81 (t, NH, 2H), 3.18-3.11 (m, 4H), 3.06 (s, 4H), 3.03-2.98 (m, 6H), 2.72-2.71 (t, 1H), 2.45-2.42 (m, 2H), 2.18-2.11 (td, 2H), 2.04-2.00 (t, 2H), 1.52-1.30 (m, 6H), 1.37 (s, 18H), 1.25-1.20 (m, 18H).

### Intermediate 96

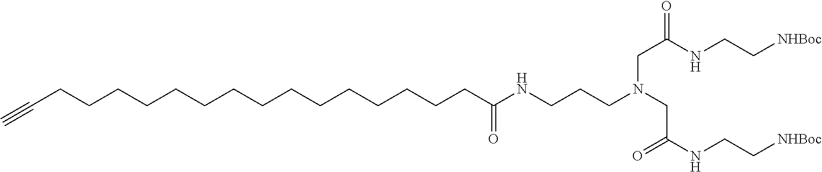 [SMILES: C#CCCCCCCCCCCCCCCCC(=O)NCCCN(CC(=O)NCCNC(=O)OC(C)(C)C)CC(=O)NCCNC(=O)OC(C)(C)C]

MS (ESI) m/z 737.6 [M+H]⁺. ¹H NMR (400 MHZ, DMSO-d₆): δ 8.07 (t, NH, 2H), 7.73 (t, NH, 1H), 6.80 (t, NH, 2H), 3.15-3.11 (m, 4H), 3.06 (s, 4H), 3.03-2.98 (m, 6H), 2.72-2.71 (t, 1H), 2.45-2.42 (m, 2H), 2.18-2.11 (td, 2H), 2.04-2.00 (t, 2H), 1.52-1.30 (m, 6H), 1.37 (s, 18H), 1.25-1.20 (m, 22H).

### Intermediate 97

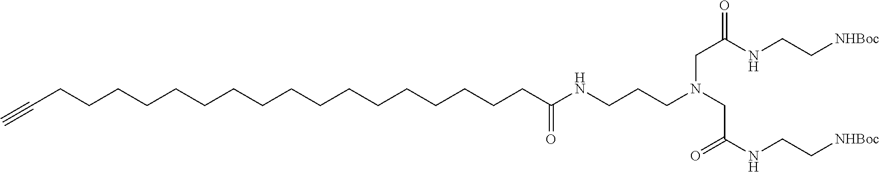 [SMILES: C#CCCCCCCCCCCCCCCCCCC(=O)NCCCN(CC(=O)NCCNC(=O)OC(C)(C)C)CC(=O)NCCNC(=O)OC(C)(C)C]

MS (ESI) m/z 765.6 [M+H]⁺. ¹H NMR (400 MHZ, DMSO-d₆): δ 8.07 (t, NH, 2H), 7.73 (t, NH, 1H), 6.80 (t, NH, 2H), 3.14-3.09 (m, 4H), 3.06 (s, 4H), 3.03-2.98 (m, 6H), 2.72-2.71 (t, 1H), 2.45-2.42 (m, 2H), 2.18-2.11 (td, 2H), 2.04-1.99 (t, 2H), 1.52-1.30 (m, 6H), 1.37 (s, 18H), 1.25-1.20 (m, 26H).

### Intermediate 98

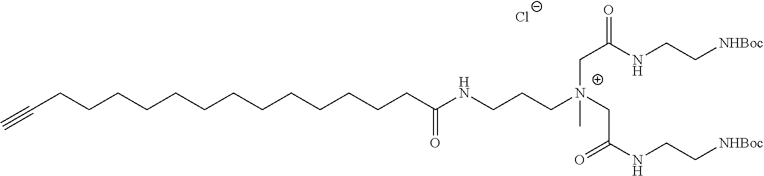 [SMILES: C#CCCCCCCCCCCCCCC(=O)NCCC[N+](C)(CC(=O)NCCNC(=O)OC(C)(C)C)CC(=O)NCCNC(=O)OC(C)(C)C ; [Cl-]]

MS (ESI) m/z 723.5 [M]⁺. ¹H NMR (400 MHZ, DMSO-d₆): δ 8.63 (t, NH, 2H), 7.91 (t, NH, 1H), 6.86 (t, NH, 2H), 4.21 (s, 4H), 3.64-3.60 (m, 2H), 3.28 (s, 3H), 3.13-3.08 (m, 6H), 3.04-3.00 (m, 4H), 2.72-2.71 (t, 1H), 2.15-2.11 (td, 2H), 2.06-2.02 (t, 2H), 1.86-1.82 (m, 2H), 1.49-1.27 (m, 4H), 1.37 (s, 18H), 1.25-1.20 (m, 18H).

### Intermediate 99

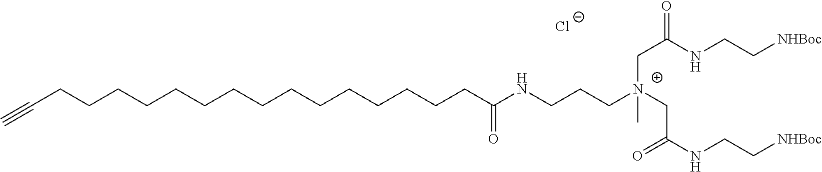 [SMILES: C#CCCCCCCCCCCCCCCCC(=O)NCCC[N+](C)(CC(=O)NCCNC(=O)OC(C)(C)C)CC(=O)NCCNC(=O)OC(C)(C)C ; [Cl-]]

MS (ESI) m/z 751.6 [M]⁺. ¹H NMR (400 MHz, DMSO-d₆): δ 8.64 (t, NH, 2H), 7.92 (t, NH, 1H), 6.86 (t, NH, 2H), 4.21 (s, 4H), 3.64-3.60 (m, 2H), 3.28 (s, 3H), 3.13-3.08 (m, 6H), 3.06-3.03 (m, 4H), 2.73-2.72 (t, 1H), 2.15-2.11 (td, 2H), 2.06-2.02 (t, 2H), 1.85-1.81 (m, 2H), 1.49-1.30 (m, 4H), 1.37 (s, 18H), 1.25-1.20 (m, 22H).

### Intermediate 100

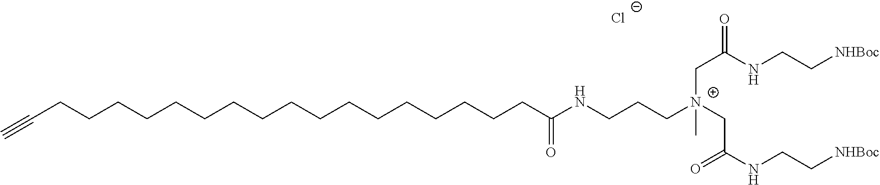 [SMILES: [Cl-] ; C#CCCCCCCCCCCCCCCCCCC(=O)NCCC[N+](C)(CC(=O)NCCNC(=O)OC(C)(C)C)CC(=O)NCCNC(=O)OC(C)(C)C]

MS (ESI) m/z 779.6 [M+H]⁺. ¹H NMR (400 MHZ, DMSO-d₆): δ 8.63 (t, NH, 2H), 7.92 (t, NH, 1H), 6.86 (t, NH, 2H), 4.21 (s, 4H), 3.64-3.60 (m, 2H), 3.28 (s, 3H), 3.13-3.08 (m, 6H), 3.06-3.03 (m, 4H), 2.73-2.72 (t, 1H), 2.15-2.11 (td, 2H), 2.06-2.02 (t, 2H), 1.85-1.81 (m, 2H), 1.49-1.30 (m, 4H), 1.37 (s, 18H), 1.25-1.20 (m, 26H).

### Intermediate 101

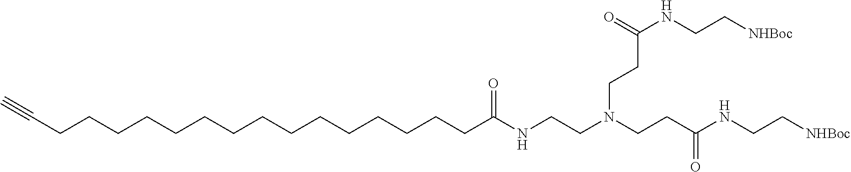 [SMILES: C#CCCCCCCCCCCCCCCCC(=O)NCCN(CCC(=O)NCCNC(=O)OC(C)(C)C)CCC(=O)NCCNC(=O)OC(C)(C)C]

MS (ESI) m/z 751.6 [M+H]⁺. ¹H NMR (400 MHZ, DMSO-d₆): δ 7.91-7.87 (t, NH, 2H), 7.60-7.56 (t, NH, 1H), 6.79-6.76 (t, NH, 2H), 3.08-3.02 (m, 6H), 2.98-2.94 (m, 4H), 2.74-2.72 (t, 1H), 2.66-2.61 (m, 4H), 2.43-2.39 (m, 2H), 2.19-2.10 (m, 6H), 2.05-1.98 (t, 2H), 1.48-1.38 (m, 4H), 1.37 (s, 18H), 1.30-1.17 (m, 22H).

### Intermediate 102

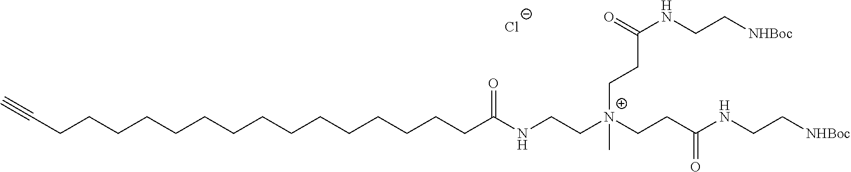 [SMILES: C#CCCCCCCCCCCCCCCCC(=O)NCC[N+](C)(CCC(=O)NCCNC(=O)OC(C)(C)C)CCC(=O)NCCNC(=O)OC(C)(C)C ; [Cl-]]

MS (ESI) m/z 765.6 [M]⁺. ¹H NMR (400 MHZ, DMSO-d₆): δ 8.16 (t, NH, 2H), 8.10 (t, NH, 1H), 6.83 (t, NH, 2H), 3.69-3.55 (m, 4H), 3.45-3.42 (m, 2H), 3.30-3.26 (m, 2H), 3.11-3.07 (m, 4H), 3.01 (s, 3H), 3.01-2.97 (m, 4H), 2.72-2.70 (t, 1H), 2.65-2.60 (m, 4H), 2.16-2.07 (m, 4H), 1.50-1.30 (m, 4H), 1.38 (s, 18H), 1.30-1,20 (m, 20H).

### Intermediate 103

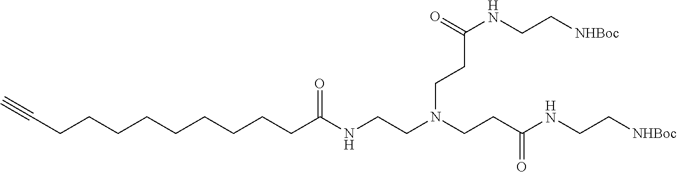 [SMILES: C#CCCCCCCCCCC(=O)NCCN(CCC(=O)NCCNC(=O)OC(C)(C)C)CCC(=O)NCCNC(=O)OC(C)(C)C]

MS (ESI) m/z 667.4 [M+H]⁺. ¹H NMR (400 MHZ, DMSO-d₆): δ 7.90-7.88 (t, NH, 2H), 7.60-7.57 (t, NH, 1H), 6.79-6.76 (t, NH, 2H), 3.08-3.03 (m, 6H), 2.98-2.93 (m, 4H), 2.74-2.72 (t, 1H), 2.66-2.61 (m, 4H), 2.43-2.39 (m, 2H), 2.19-2.11 (m, 6H), 2.06-1.99 (t, 2H), 1.46-1.33 (m, 4H), 1.33 (s, 18H), 1.28-1.17 (m, 10H).

### Intermediate 104

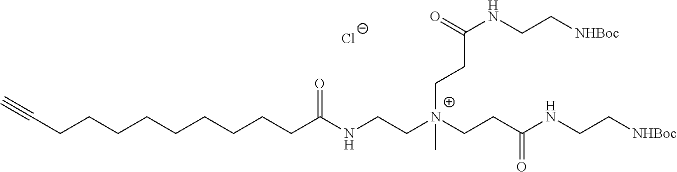 [SMILES: C#CCCCCCCCCCC(=O)NCC[N+](C)(CCC(=O)NCCNC(=O)OC(C)(C)C)CCC(=O)NCCNC(=O)OC(C)(C)C ; [Cl-]]

MS (ESI) m/z 681.5 [M]⁺. ¹H NMR (400 MHZ, DMSO-d₆): δ 8.16 (t, NH, 2H), 8.10 (t, NH, 1H), 6.84 (t, NH, 2H), 3.69-3.55 (m, 4H), 3.48-3.42 (m, 2H), 3.30-3.26 (m, 2H), 3.10-3.06 (m, 4H), 3.01 (s, 3H), 3.01-2.93 (m, 4H), 2.74-2.73 (t, 1H), 2.64-2.60 (m, 4H), 2.16-2.07 (m, 4H), 1.50-1.30 (m, 4H), 1.38 (s, 18H), 1.30-1,20 (m,10H).

### Intermediate 105

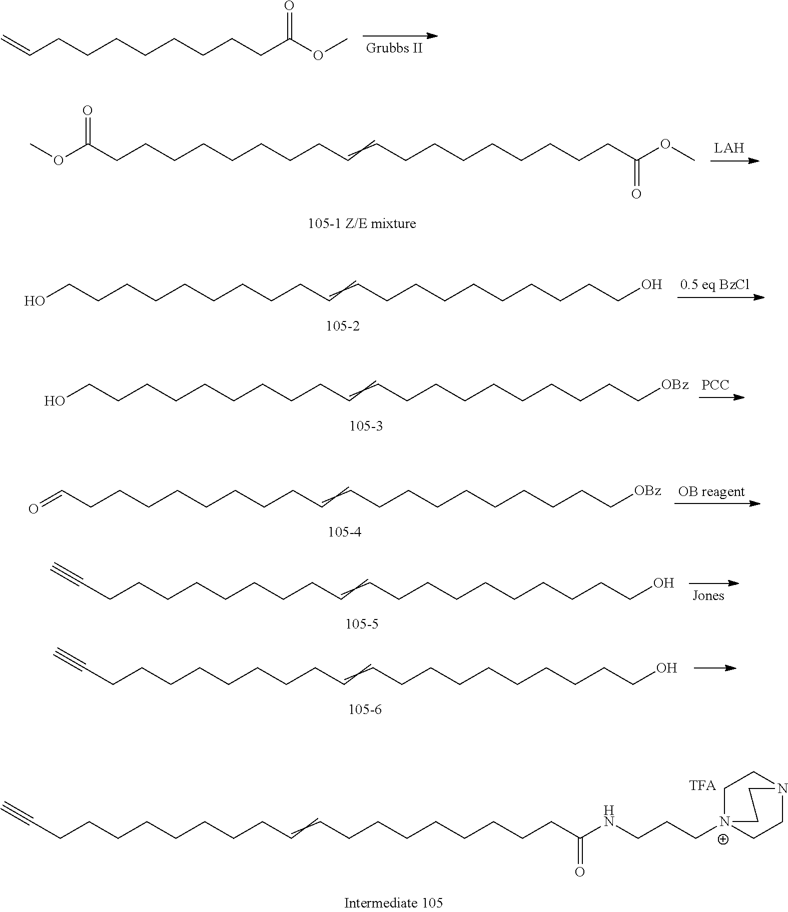

Step 1. A flask charged with methyl undec-10-enoate (10.0 g, 50.5 mmol) and Grubbs catalyst (2nd generation)(1.0 g, 1.18 mmol) in toluene (200 mL) was degassed 3 times and filled with N2. The resulting mixture was then heated at 80° C. for 16 hrs. Solvent was removed and the residue was purified by silica gel column chromatography (PE/EA=40:1 to 10:1 to 5:1) to give dimethyl icos-10-enedioate, 105-1, (6.2 g, yield: 66.7%) as pale-yellow solid. ¹H NMR (400 MHZ, DMSO-d₆) δ 5.36-5.30 (m, 2H), 3.57 (s, 6H), 2.27 (t, J=7.2 Hz, 4H), 1.98-1.91 (m, 4H), 1.51-1.48 (m, 4H), 1.29-1.18 (m, 20H).

Step 2. To a mixture of 105-1 (1.0 g, 2.82 mmol) in dry THF (40 mL) was added LAH (430 mg, 11.3 mmol) in portions under N₂ atmosphere at 0° C. After addition, the resulting mixture was stirred for 30 min at r. t. and heated at 50° C. for additional 12 hrs. The reaction mixture was cooled to 0° C., quenched with water (0.43 mL), aq. NaOH (15%, 0.43 mL) and water (1.29 mL) carefully, and filtered. The filter cake was washed with hot THF (20 mL×3) and the organic phase was combined, dried and concentrated to give crude icos-10-ene-1,20-diol, 105-2, (1.02 g) as yellow solid, which was directly used in the next step. ¹H NMR (400 MHZ, DMSO-d₆) δ 5.36-5.31 (m, 2H), 4.29 (t, J=4.8 Hz, 2H), 3.38-3.34 (m, 4H), 1.99-1.91 (m, 4H), 1.44-1.35 (m, 4H), 1.29-1.22 (m, 24H).

Step 3. To a mixture of 105-2, (1.02 g, 2.82 mmol) and TEA (430 mg, 4.23 mmol) in dry DCM (20 mL) was added benzoyl chloride (200 mg, 1.41 mmol) dropwise at r. t. After addition, the resulting mixture was stirred for 12 hrs at r. t. Solvent was removed and the residue was purified by silica gel column chromatography (PE/EA=20:1 to 10:1 to 1:1) to give 20-hydroxyicos-10-en-1-yl benzoate, 105-3, (398 mg, yield: 33.9% over 2 steps) as white solid. ¹H NMR (400 MHZ, DMSO-d₆) δ 7.95 (d, J=7.2 Hz, 2H), 7.65 (t, J=7.2 Hz, 1H), 7.52 (t, J=7.6 Hz, 2H), 5.39-5.30 (m, 2H), 4.30-4.24 (m, 2H), 3.38-3.31 (m, 2H), 1.98-1.92 (m, 4H), 1.73-1.66 (m, 2H), 1.36-1.32 (m, 2H), 1.20-1.15 (m, 24H).

Step 4. To a mixture of crude 105-3 (1.5 g, 3.6 mmol) in DCM (20 mL) was added PCC (1.55 g, 7.2 mmol) in portions at r. t. After addition, the resulting mixture was stirred for 14 hrs at r. t. The reaction mixture was filtered over celite and concentrated. The residue was purified by silica gel column chromatography (PE/EA=10:1) to give 20-oxoicos-10-en-1-yl benzoate, 105-4, (679 mg, yield: 45.5%) as white solid. ¹H NMR (400 MHz, DMSO-d₆) δ 9.64 (s, 1H), 7.96-7.94 (m, 2H), 7.65 (t, J=7.2 Hz, 1H), 7.54-7.51 (m, 2H), 5.38-5.30 (m, 2H), 4.26 (t, J=6.4 Hz, 2H), 2.40-2.37 (m, 2H), 1.98-1.93 (m, 4H), 1.51-1.47 (m, 2H), 1.39-1.23 (m, 22H).

Step 5. To a mixture of 105-4 (150 mg, 0.362 mmol) in MeOH (3 mL) was added Ohira-Bestmann reagent (105 mg, 0.546 mmol) and K₂CO₃ (75 mg, 0.546 mmol) at r. t. The reaction mixture was then heated at 30° C. for 12 hrs. Solvent was removed and the residue was purified by silica gel column chromatography (PE/EA=5:1) to give henicos-10-en-20-yn-1-ol, 105-5, (70 mg, yield: 63%) as white solid. ¹H NMR (400 MHZ, DMSO-d₆) δ 5.37-5.31 (m, 2H), 4.30 (t, J=5.2 Hz, 1H), 3.38-3.34 (m, 2H), 2.71 (t, J=2.4 Hz, 1H), 2.15-2.11 (m, 2H), 1.98-1.93 (m, 4H), 1.44-1.37 (m, 4H), 1.35-1.23 (m, 22H).

Step 6. To a mixture of 105-5 (70 mg, 0.228 mmol) in (2 mL) was added Jones reagent (0.028 mL, 0.457 mmol) at 0° C. The reaction mixture was stirred at room temperature for 30 min. TLC indicated the completion of reaction. Acetone was removed and the mixture was diluted with H₂O (10 mL), extracted with EA (15 mL×3). The organic layers were combined, dried and concentrated to give crude henicos-10-en-20-ynoic acid, 105-6, (66 mg, yield: 90.3%) as pale-yellow solid. ¹H NMR (400 MHZ, DMSO-d₆) δ 11.93 (s, 1H), 5.37-5.31 (m, 2H), 2.71 (t, J=2.4 Hz, 1H), 2.15-2.11 (m, 4H), 1.98-1.93 (m, 4H), 1.49-1.38 (m, 4H), 1.29-1.19 (m, 20H).

Step 7. To a mixture of crude 105-6 (100 mg, 0.312 mmol), 1-(3-aminopropyl)-1,4-diazabicyclo[2.2.2]octan-1-ium (212 mg, ˜70% purity, 0.875 mmol) and TEA (64 mg, 0.625 mmol) in DMF (3 mL) was added HATU (142 mg, 0.375 mmol). The resulting mixture was stirred at 30° C. for 6 hrs. DMF was removed at reduced pressure and the residue was diluted with EA (15 mL), washed with water (10 mL×3) and brine, dried and concentrated to give crude 1-(3-(henicos-10-en-20-ynamido) propyl)-1,4-diazabicyclo[2.2.2]octan-1-ium, Intermediate 105, (120 mg, yield: 81.6%) as grey solid. MS (ESI) m/z 472.5 [M+]⁺. ¹H NMR (400 MHZ, DMSO-d₆) δ 7.88 (s, 1H), 5.36-5.31 (m, 2H), 3.27-3.21 (m, 6H), 3.18-3.07 (m, 4H), 3.05-2.98 (m, 6H), 2.70-2.69 (m, 1H), 2.15-2.11 (m, 2H), 2.07-2.03 (m, 2H), 1.99-1.90 (m, 4H), 1.82-1.76 (m, 2H), 1.51-1.38 (m, 4H), 1.36-1.19 (m, 20H).

### Synthesis of Examples

### Example 1

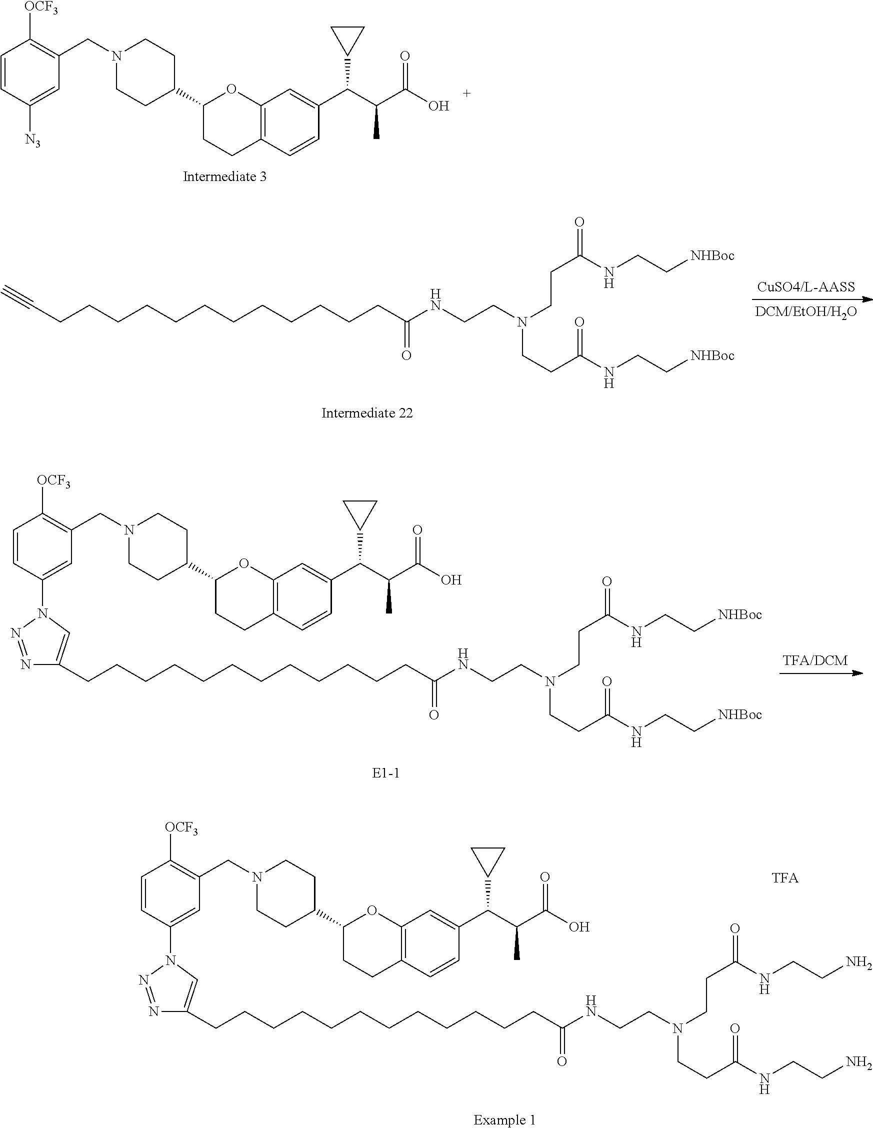 [SMILES: C[C@H](C(=O)O)[C@H](c1ccc2c(c1)O[C@@H](C1CCN(Cc3cc(N=[N+]=[N-])ccc3OC(F)(F)F)CC1)CC2)C1CC1 ; C#CCCCCCCCCCCCCC(=O)NCCN(CCC(=O)NCCNC(=O)OC(C)(C)C)CCC(=O)NCCNC(=O)OC(C)(C)C ; C[C@H](C(=O)O)[C@H](c1ccc2c(c1)O[C@@H](C1CCN(Cc3cc(-n4cc(CCCCCCCCCCCCC(=O)NCCN(CCC(=O)NCCNC(=O)OC(C)(C)C)CCC(=O)NCCNC(=O)OC(C)(C)C)nn4)ccc3OC(F)(F)F)CC1)CC2)C1CC1 ; C[C@H](C(=O)O)[C@H](c1ccc2c(c1)O[C@@H](C1CCN(Cc3cc(-n4cc(CCCCCCCCCCCCC(=O)NCCN(CCC(=O)NCCN)CCC(=O)NCCN)nn4)ccc3OC(F)(F)F)CC1)CC2)C1CC1]

Step 1. To a mixture of Intermediate 3 (18.4 mg, 0.033 mmol), Intermediate 22 (26.5 mg, 0.037 mmol) in EtOH (0.5 mL) and DCM (0.5 mL) was added CuSO₄·5H₂O (0.1 M in water, 0.187 mL) and L-AASS (0.2 M in water, 0.187 mL) at r. t. The resulting mixture was then stirred for 16 hrs. The reaction mixture was diluted with water and extracted with DCM twice. The organic phases were washed with brine, dried with Na₂SO₄, filtered, and the filtrate was concentrated to give (2S,3R)-3-((R)-2-(1-(5-(4-(12-(3-((2-((tert-butoxycarbonyl)amino)ethyl)amino)-3-oxopropyl)-2,2-dimethyl-4,9,16-trioxo-3-oxa-5,8,12,15-tetraazaoctacosan-28-yl)-1H-1,2,3-triazol-1-yl)-2-(trifluoromethoxy)benzyl) piperidin-4-yl) chroman-7-yl)-3-cyclopropyl-2-methylpropanoic acid, E1-1, (25 mg, crude) as a yellow gum. MS (ESI) m/z 634.5 [(M+2H)/2].

Step 2. A solution of E1-1 (25 mg, 0.0197 mmol) in DCM (2 mL) and TFA (0.5 mL) was stirred for 2 hrs. Solvent was removed and the residue was purified by prep-HPLC to give (2S,3R)-3-((R)-2-(1-(5-(4-(13-((2-(bis(3-((2-aminoethyl)amino)-3-oxopropyl)amino)ethyl)amino)-13-oxotridecyl)-1H-1,2,3-triazol-1-yl)-2-(trifluoromethoxy)benzyl) piperidin-4-yl) chroman-7-yl)-3-cyclopropyl-2-methylpropanoic acid, Example 1, (6 mg, yield: 28.5%) as a yellow gum. MS (ESI) m/z 1067.4 [M+H]⁺. ¹H NMR (400 MHZ, DMSO-d₆): δ 10.01 (br, 1H), 9.89 (br, 1H), 8.63 (s, 1H), 8.48 (s, 1H), 8.45-8.42 (m, 1H), 8.17 (br, 1H), 8.12-8.10 (d, 1H), 7.93 (br, 6H), 7.75-7.72 (d, 1H), 6.97-6.95 (d, 1H), 6.66-6.64 (d, 1H), 6.51 (s, 1H), 4.47 (br, 2H), 3.81 (br, 1H), 3.5-3.3 (m, 12H), 3.32-3.29 (m, 4H), 3.2 (br, 4H), 2.89-2.87 (m, 4H), 2.73-2.64 (m, 9H), 2.11-2.07 (m, 3H), 1.96-1.81 (m, 5H), 1.68-1.65 (m, 4H), 1.49-1.48 (m, 2H), 1.32-1.24 (m, 16H), 1.07-1.02 (m, 1H), 0.77 (d, 3H), 0.52-0.48 (m, 1H), 0.25-0.21 (m, 2H), −0.10 ˜−0.14 (m, 1H).

The rest of the examples having a triazole unit were synthesized in the same procedure as for Example 1.

### Example 2

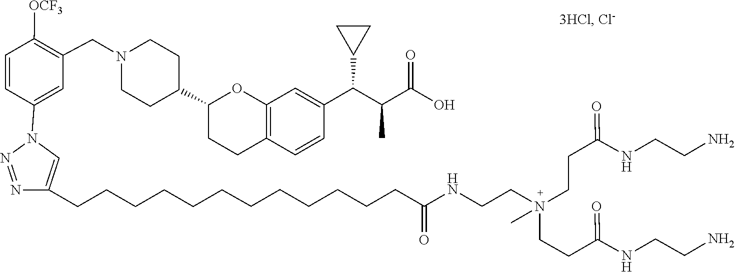

MS (ESI) m/z 1081.4 [M+]⁺. ¹H NMR (400 MHZ, DMSO-d₆): δ=9.6 (br, 1H), 8.62 (s, 1H), 8.39-8.36 (m, 1H), 8.34 (t, NH, 2H), 8.13 (t, NH, 1H), 8.12-8.10 (d, 1H), 7.81 (br, 6H), 7.75-7.73 (d, 1H), 7.00-6.98 (d, 1H), 6.67-6.65 (d, 1H), 6.51 (s, 1h), 4.51 (m, 2H), 3.80-3.77 (m, 1H), 3.59-3.54 (m, 4H), 3.5-3.12 (m, 12H), 3.03 (s, 3H), 2.89-2.84 (m, 4H), 2.73-2.64 (m, 9H), 2.11-2.07 (m, 3H), 1.95-1.80 (m, 5H), 1.70-160 (m, 4H), 1.50-1.40 (, m, 2H), 1.27-1.23 (m, 16H), 1.10-1.02 (m, 1H), 0.80-0.77 (d, 3H), 0.52-0.49 (m, 1H), 0.24-0.22 (m, 2H), −0.11 ˜−0.13.

### Example 3

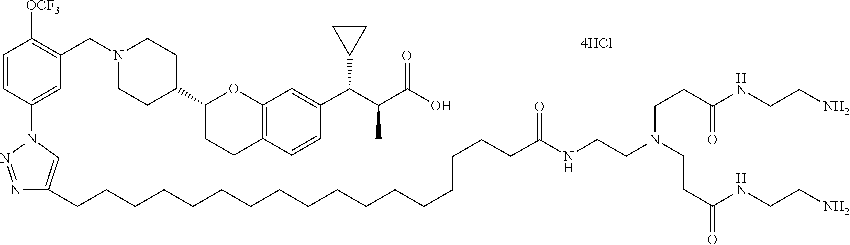 [SMILES: C[C@H](C(=O)O)[C@H](c1ccc2c(c1)O[C@@H](C1CCN(Cc3cc(-n4cc(CCCCCCCCCCCCCCCCCC(=O)NCCN(CCC(=O)NCCN)CCC(=O)NCCN)nn4)ccc3OC(F)(F)F)CC1)CC2)C1CC1]

MS (ESI) m/z 1137.5 [M+H]⁺. ¹H NMR (400 MHZ, DMSO-d₆): δ=10.8 (br, 1H), 10.4 (br, 1H), 8.71 (s, 2H), 8.53 (t, NH, 2H), 8.23 (t, NH, 1H), 8.13 (br, 7H), 7.74-7.72 (d, 1H), 6.98-6.96 (d, 1H), 6.67-6.65 (d, 1H), 6.53 (s, 1H), 4.5 (m, 2H), 3.82-3.79 (m, 1H), 3.5-3.26 (m, 12H), 1.24-1.20 (m, 4H), 2.89-2.86 (m, 4H), 2.75-2.62 (m, 9H), 2.12-2.07 (m, 3H), 2.05-1.92 (m, 1H), 1.85-1.80 (m, 4H), 1.70-1.65 (m, 4H), 1.52-1.42 (m, 2H), 1.4-1.20 (m, 26H), 1.12-1.02 (m, 1H), 1.84-1.81 (d, 3H), 0.53-0.49 (m, 1H), 0.25-0.20 (m, 2H), −0.11 ˜−0.13 (m, 1H).

### Example 4

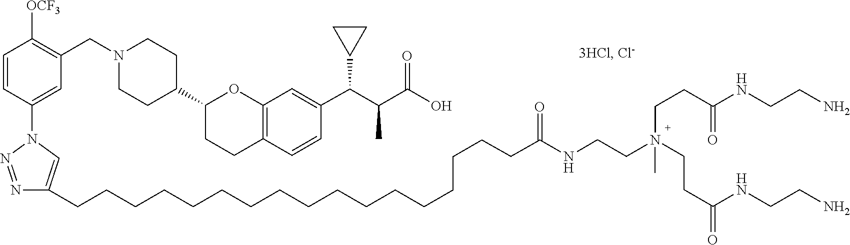

MS (ESI) m/z 1151.5 [M]⁺. ¹H NMR (400 MHZ, DMSO-d₆): δ=10.9 (br, 1H), 8.71-8.70 (br, 2H), 8.60 (t, NH, 2H), 8.30 (t, NH, 1H), 8.16 (br, 6H), 8.14-8.12 (d, 1H), 7.73-7.71 (d, 1H), 6.97-6.95 (d, 1H), 6.66-6.64 (d, 1H), 6.52 (s, 1H), 4.47 (br, 2h), 3.80-3.78 (m, 1H), 3.63-3.60 (m, 4H), 3.58-3.35 (m, 6H), 3.35-3.32 (m, 4H), 3.23-3.09 (m, 2H), 3.08 (s, 3H), 2.90-2.86 (m, 4H), 2.81-2.62 (m, 9H), 2.11-2.03 (m, 3H), 2.01-1.94 (m, 1H), 1.94-1.81 (m, 4H), 1.68-1.65 (m, 4H), 1.51-1.42 (m, 2H), 1.40-1.20 (m, 26H), 1.12-1.02 (m, 1H), 1.84-1.81 (d, 3H), 0.53-0.49 (m, 1H), 0.25-0.20 (m, 2H), −0.11 ˜−0.13 (m, 1H).

### Example 5

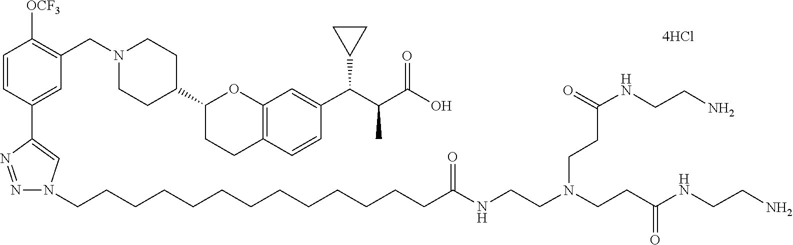 [SMILES: C[C@H](C(=O)O)[C@H](c1ccc2c(c1)O[C@@H](C1CCN(Cc3cc(-c4cn(CCCCCCCCCCCCCC(=O)NCCN(CCC(=O)NCCN)CCC(=O)NCCN)nn4)ccc3OC(F)(F)F)CC1)CC2)C1CC1]

MS (ESI) m/z 1081.4 [M+]⁺. ¹H NMR (400 MHZ, DMSO-d₆): δ=10.5 (br, 1H), 10.4 (br, 1H), 8.67 (s, 1H), 8.55 (s, 1H), 8.53-8.50 (t, 2H), 8.24-822 (m, 1H), 8.11 (br, 6H), 8.04-8.02 (d, 1H), 7.59-7.57 (d, 1H), 6.97-6.95 (d, 1H), 6.66-6.64 (d, 1H), 6.52 (s, 1H), 4.5 (m, 4H), 3.82-3.81 (m, 1H), 3.46-3.43 (m, 2H), 3.38-3.35 (m, 6H), 3.34-3.31 (m, 4H), 3.16-3.11 (m, 4H), 2.89-2.86 (m, 4H), 2.78-2.62 (m, 7H), 2.11-2.05 (m, 3H), 1.97-1.76 (m, 7H), 1.64-1.62 (m, 1H), 1.48 (br, 3H), 1.31-1.12 (m, 18H), 1.09-1.00 (m, 1H), 0.79 (d, 3H), 0.51-0.49 (m, 1H), 0.25-0.23 (m, 2H), −0.12˜−0.14 (m, 1H).

### Example 6

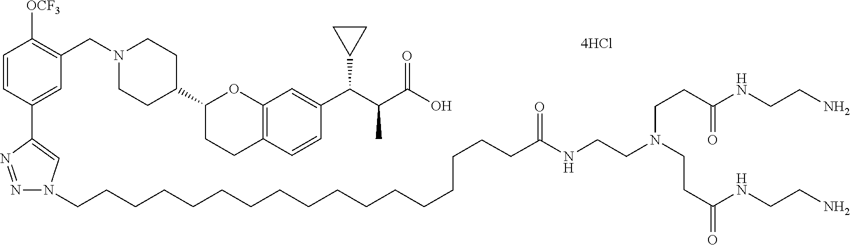 [SMILES: C[C@H](C(=O)O)[C@H](c1ccc2c(c1)O[C@@H](C1CCN(Cc3cc(-c4cn(CCCCCCCCCCCCCCCCCC(=O)NCCN(CCC(=O)NCCN)CCC(=O)NCCN)nn4)ccc3OC(F)(F)F)CC1)CC2)C1CC1]

MS (ESI) m/z 569.3 [(M+2H)/2]⁺. ¹H NMR (400 MHZ, DMSO-d₆) δ: 10.74 (brs, 1H), 10.50 (brs, 1H), 8.67 (s, 1H), 8.55 (d, J=2.0 Hz, 1H), 8.51 (t, J=5.6 Hz, 2H), 8.23 (t, J=5.6 Hz, 1H), 8.11 (brs, 6H), 8.03 (dd, J=2.4, 8.8 Hz, 1H), 7.58 (d, J=7.2 Hz, 1H), 6.96 (d, J=7.6 Hz, 1H), 6.64 (d, J=7.6 Hz, 1H), 6.52 (s, 1H), 4.43 (t, J=6.4 Hz, 4H), 3.80 (dd, J=3.2, 9.6 Hz, 1H), 339-3.29 (m, 12H), 3.16-3.11 (m, 4H), 2.88 (q, J=5.6 Hz, 4H), 2.77-2.60 (m, 7H), 2.10 (t, J=7.2 Hz, 3H), 1.97-1.76 (m, 8H), 1.68-1.61 (m, 1H), 1.52-1.46 (m, 2H), 1.27-1.22 (m, 26H), 1.09-1.03 (m, 1H), 0.80 (d, J=6.8 Hz, 3H), 0.52-0.49 (m, 1H), 0.25-0.23 (m, 2H), −0.08−−0.12 (m, 1H).

### Example 7

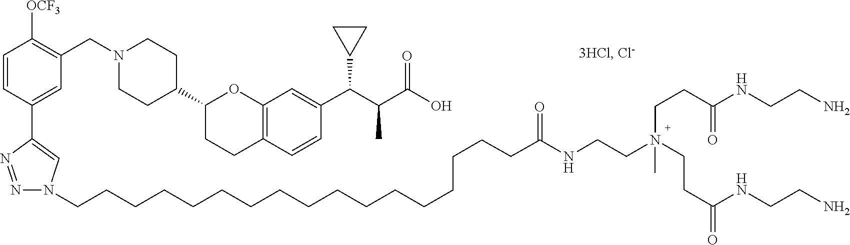

MS (ESI) m/z 1152.8 [M+H]⁺. ¹H NMR (400 MHz, DMSO-d₆): δ=9.82 (br, 1H), 8.63 (s, 1H), 8.40-8.37 (t, 2H), 8.31 (s, 1H), 8.16 (t, 1H), 8.01-7.98 (d, 1H), 7.91 (brs, 4H), 7.59 (d, 1H), 6.94 (d, 1H), 6.65 (d, 1H), 6.52 (s, 1H), 4.44-4.41 (m, 4H), 3.84-3.82 (m, 1H), 3.60-3.57 (m, 4H), 3.56-3.36 (m, 6H), 3.35-3.30 (m, 4H), 3.22-3.10 (m, 2H), 3.04 (s, 3H), 2.91-2.87 (m, 4H), 2.75-2.60 (m, 7H), 2.11-2.07 (m, 3H), 1.91-1.81 (m, 6H), 1.70-1.60 (m, 3H), 1.50-1.42 (m, 2H), 1.30-1.18 (m, 26H), 1.06-0.98 (m, 1H), 0.80 (d, 3H), 0.52-0.47 (m, 1H), 0.26-0.19 (m, 2H), −0.09−−0.10 (m, 1H).

### Example 8

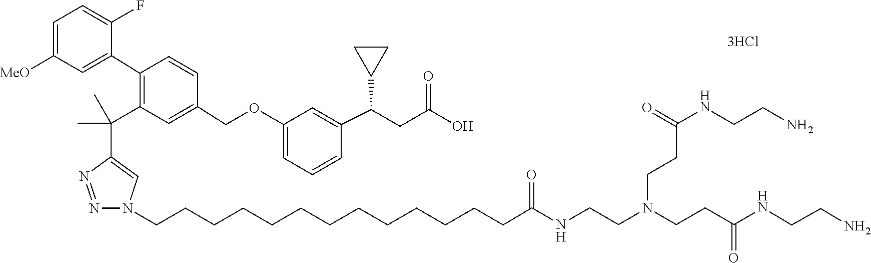 [SMILES: COc1ccc(F)c(-c2ccc(COc3cccc([C@@H](CC(=O)O)C4CC4)c3)cc2C(C)(C)c2cn(CCCCCCCCCCCCCC(=O)NCCN(CCC(=O)NCCN)CCC(=O)NCCN)nn2)c1]

MS (ESI) m/z 1027.4 [M+H]⁺. ¹H NMR (400 MHZ, DMSO-d₆): δ=9.55 (br, 1H), 8.38 (t, NH, 2H), 8.13 (t, NH, 1H), 7.85 (br, 6H), 7.69 (s, 1H), 7.35 (d, 1H), 7.30 (s, 1H), 7.25-7.21 (t, 1H), 7.01-6.85 (m, 5H), 6.81-6.77 (m, 1H), 5.96-5.94 (m, 1H), 5.15 (s, 2H), 4.11-4.05 (m, 2H), 3.60 (s, 3H), 3.5-3.3 (m, 10H), 3.22-3.16 (m, 2H), 2.89-2.86 (m, 4H), 2.70-2.62 (m, 6H), 2.30-2.26 (m, 1H), 2.10-2.07 (m, 2H), 1.75-1.73 (m, 2H), 1.60 (s, 3H), 1.47 (s, 3H), 1.48-1.46 (m, 2H), 1.28-1.20 (m, 18H), 1.06-1.00 (m, 1H), 0.51-0.45 (m, 1H), 0.3-0.2 (m, 2H), 0.13-0.10 (m, 1H).

### Example 9

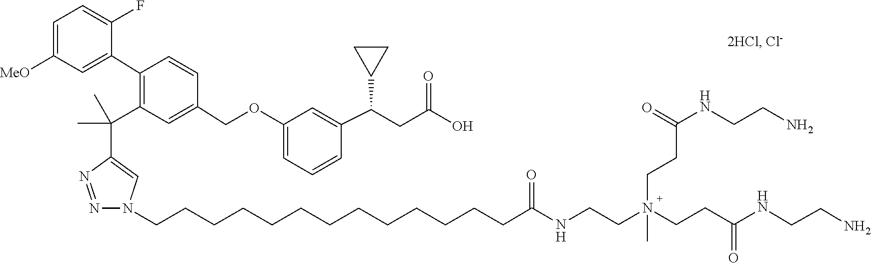

MS (ESI) m/z 1041.4 [M]⁺. ¹H NMR (400 MHz, DMSO-d₆): δ=8.62 (t, NH, 2H), 8.35 (t, NH, 1H), 8.22 (br, 6H), 7.69 (s, 1H), 7.35-7.32 (d, 1H), 7.32 (s, 1H), 7.22 (t, 1H), 7.00-6.85 (m, 5H), 6.81-6.78 (m, 1H), 5.94-5.93 (m, 1H), 5.13 (s, 2H), 4.14-4.01 (m, 2H), 3.64-3.60 (m, 4H), 3.59 (s, 3H), 3.47-3.45 (m, 2H), 3.40-3.33 (m, 6H), 3.07 (s, 3H), 2.89-2.87 (m, 4H), 2.75-2.71 (m, 4H), 2.67-2.60 (m, 2H), 2.29-2.23 (m, 1H), 2.11-2.08 (m, 2H), 1.72-1.69 (m, 2H), 1.59 (s, 3H), 1.49-1.46 (m, 2H), 1.46 (s, 3H), 1.02-1.00 (m, 1H), 0.50-0.48 (m, 1H), 0.3-0.23 (m, 2H), 0.15-0.11 (m, 1H).

### Example 10

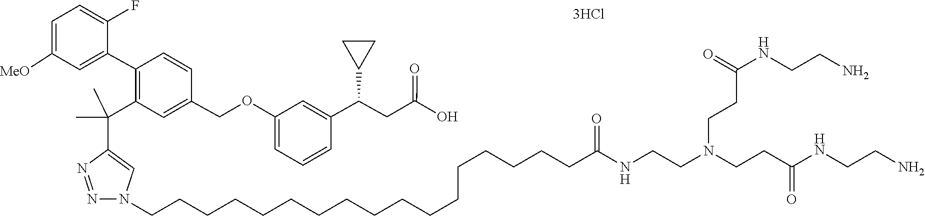 [SMILES: COc1ccc(F)c(-c2ccc(COc3cccc([C@@H](CC(=O)O)C4CC4)c3)cc2C(C)(C)c2cn(CCCCCCCCCCCCCCCCCC(=O)NCCN(CCC(=O)NCCN)CCC(=O)NCCN)nn2)c1]

MS (ESI) m/z 1282.9 [M+H]⁺. ¹H NMR (400 MHZ, DMSO-d₆): δ=9.59 (br, 1H), 8.41 (t, 2H), 8.15 (t, 1H), 7.89 (brs, —NH₃⁺, 6H), 7.69 (s, 1H), 7.35 (d, J=8.0 Hz, 1H), 7.31 (s, 1H), 7.23 (t, J=8.0 Hz, 1H), 7.01-6.96 (m, 1H), 6.96-6.93 (m, 2H), 6.91-6.86 (m, 2H), 6.81-6.77 (m, 1H), 5.97-5.94 (m, 1H), 5.14 (s, 2H), 4.14-4.04 (m, 2H), 3.60 (s, 3H), 3.43-3.37 (m, 6H), 3.34-3.29 (m, 4H), 3.19-3.18 (m, 2H), 2.91-2.86 (m, 4H), 2.70-2.61 (m, 6H), 2.29-2.27 (m, 1H), 2.11-2.07 (m, 2H), 1.75-1.70 (m, 2H), 1.60 (s, 3H), 1.50-1.47 (m, 5H), 1.35-1.15 (m, 26H), 1.05-1.00 (m, 1H), 0.53-0.47 (m, 1H), 0.34-0.23 (m, 2H), 0.15-0.10 (m, 1H).

### Example 11

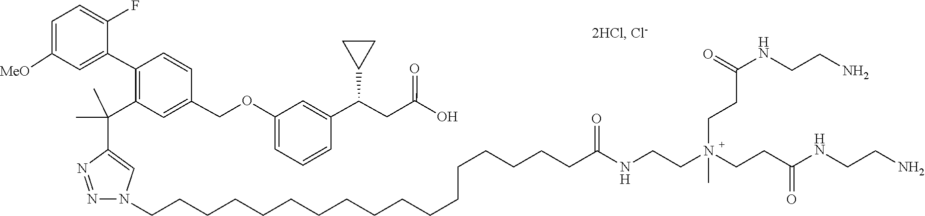

MS (ESI) m/z 549 [(M+H)/2]⁺. ¹H NMR (400 MHZ, DMSO-d₆): δ=8.40 (t, 2H), 8.16 (t, 1H), 7.91 (brs, —NH₃⁺,6H), 7.69 (s, 1H), 7.36-7.34 (d, 1H), 7.30 (s, 1H), 7.25-7.21 (t, 1H), 7.04-6.98 (t, 1H), 6.98-6.95 (m, 2H), 6.93-6.85 (m, 2H), 6.81-6.77 (m, 1H), 5.96-5.93 (m, 1H), 5.14 (s, 2H), 4.13-4.02 (m, 2H), 3.70 (s, 3H), 3.70-3.56 (m, 4H), 3.49-3.43 (m, 2H), 3.34-3.29 (m, 6H), 3.03 (s, 3H), 2.90-2.86 (m, 4H), 2.71-2.61 (m, 6H), 2.30-2.24 (m, 1H), 2.11-2.07 (t, J=7.2 Hz, 2H), 1.75-1.70 (m, 2H), 1.60 (s, 3H), 1.49-1.44 (m, 5H), 1.27-1.19 (m, 26H), 1.05-0.99 (m, 1H), 0.53-0.47 (m, 1H), 0.34-0.21 (m, 2H), 0.15-0.10 (m, 1H).

### Example 12

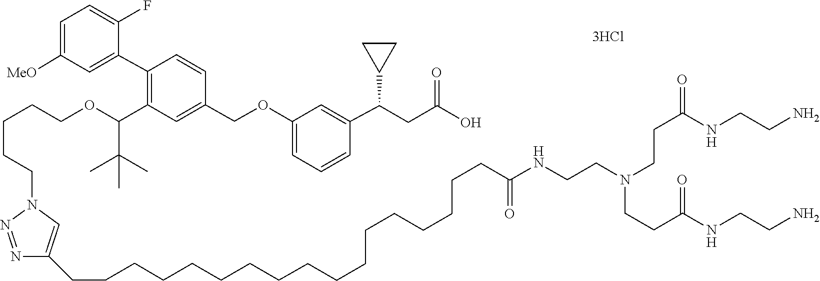 [SMILES: COc1ccc(F)c(-c2ccc(COc3cccc([C@@H](CC(=O)O)C4CC4)c3)cc2C(OCCCCCn2cc(CCCCCCCCCCCCCCCCCC(=O)NCCN(CCC(=O)NCCN)CCC(=O)NCCN)nn2)C(C)(C)C)c1]

MS (ESI) m/z 1197.6 [M+H]⁺. ¹H NMR (400 MHZ, DMSO-d₆)_{8=10.45} (br, 1H), 8.55 (t, NH, 2H), 8.25 (t, NH, 1H), 8.18 (brs, 6H), 7.82 (s, 1H), 7.55-7.41 (m, 3H), 7.34 (s, 1H), 7.24-7.16 (m, 4H), 7.00-6.96 (m, 1H), 6.90 (s, 1H), 6.86-6.81 (m, 2H), 6.79-6.69 (m, 1H), 5.17 (s, 2H), 4.29 (t, 2H), 4.18, 3.93 (s, s, isomers, 1H), 3.74 (s, 3H), 3.49-3.46 (m, 2H), 3.39-3.32 (m, 9H), 3.28-3.23 (m, 3H), 2.91-2.87 (m, 4H), 2.69 (t, 4H), 2.62 (m, 2H), 2.56 (t, 2H), 2.28-2.22 (m, 1H), 2.10 (t, 2H), 1.81-1.75 (m, 2H), 1.58-1.43 (m, 6H), 1.29-1.19 (m, 28H), 1.01-0.94 (m, 1H), 0.61 (s, 9H), 0.49-0.44 (m, 1H), 0.28-0.19 (m, 2H), 0.09-0.04 (m, 1H).

### Example 13

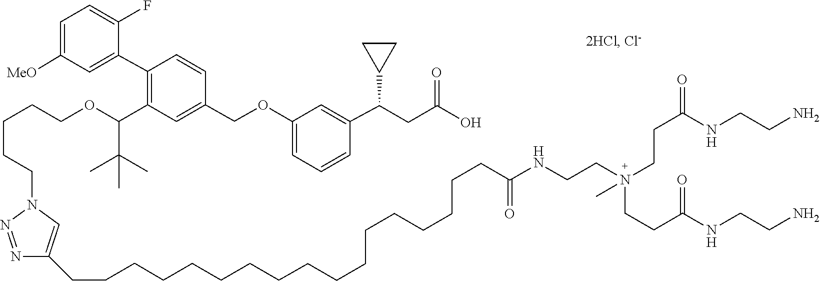

MS (ESI) m/z 1197.6 [M+]⁺. ¹H NMR (400 MHZ, DMSO-d₆): δ 8.64 (t, NH, 2H), 8.37 (t, NH, 1H), 8.26 (brs, 6H), 7.84 (s, 1H), 7.55-7.35 (m, 2H), 7.24-7.14 (m, 3H), 6.98-6.94 (m, 1H), 6.88 (s, 1H), 6.84-6.70 (m, 3H), 5.15 (s, 2H), 4.28 (t, 2H), 4.16, 3.91 (s, s, 1H), 3.72 (s, 3H), 3.64-3.60 (m, 4H), 3.47-3.42 (m, 2H), 3.37-3.31 (m, 6H), 3.27-3.13 (m, 2H), 3.07 (s, 3H), 2.90-2.81 (m, 4H), 2.75-2.70 (m, 4H), 2.63-2.53 (m, 4H), 2.26-2.19 (m, 1H), 2.09 (t, 2H), 1.80-1.74 (m, 2H), 1.57-1.42 (m, 6H), 1.27-1.16 (m, 28H), 0.99-0.92 (m, 1H), 0.59 (s, 9H), 0.47-0.42 (m, 1H), 0.26-0.17 (m, 2H), 0.07-0.02 (m, 1H).

### Example 14

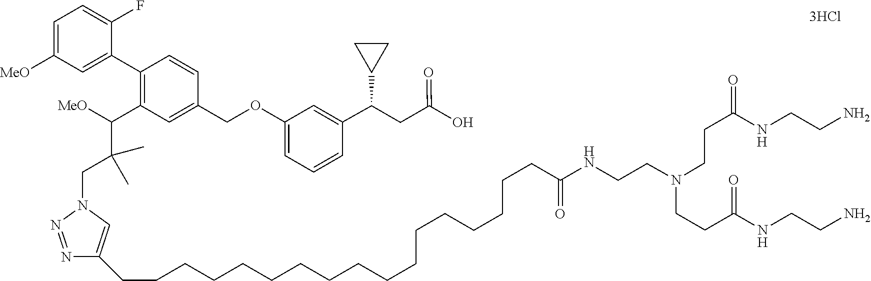 [SMILES: COc1ccc(F)c(-c2ccc(COc3cccc([C@@H](CC(=O)O)C4CC4)c3)cc2C(OC)C(C)(C)Cn2cc(CCCCCCCCCCCCCCCCCC(=O)NCCN(CCC(=O)NCCN)CCC(=O)NCCN)nn2)c1]

MS (ESI) m/z 570.9 [(M+2H)/2]⁺.

### Example 15

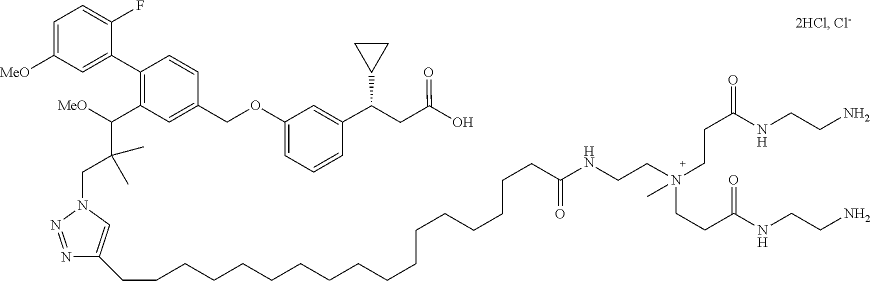

MS (ESI) m/z 1155.5 [M+]⁺.

### Example 16

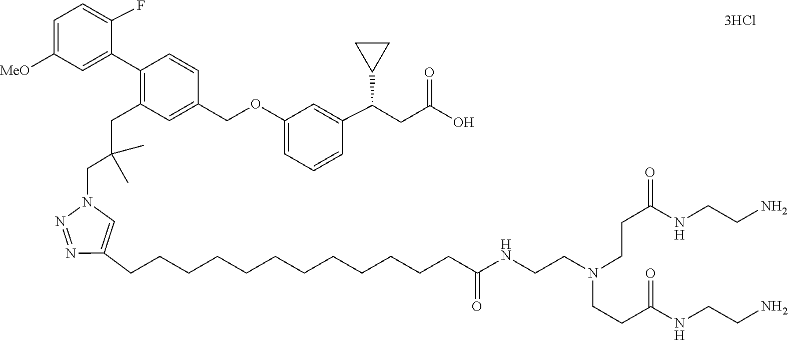 [SMILES: COc1ccc(F)c(-c2ccc(COc3cccc([C@@H](CC(=O)O)C4CC4)c3)cc2CC(C)(C)Cn2cc(CCCCCCCCCCCCC(=O)NCCN(CCC(=O)NCCN)CCC(=O)NCCN)nn2)c1]

MS (ESI) m/z 1040.4 [M+H]⁺. ¹H NMR (400 MHZ, DMSO-d₆): δ 9.90 (br, 1H), 8.42 (t, NH, 2H), 8.16 (t, NH, 1H), 7.92 (br, 6H), 7.63 (s, 1H), 7.44 (s, 1H), 7.43-7.40 (d, 1H), 7.23-7.12 (m, 3H), 6.94-6.91 (m, 2H), 6.86-6.77 (m, 3H), 5.14 (s, 2H), 3.94 (s, 2H), 3.73 (s, 3H), 3.45-3.42 (m, 2H), 3.41-3.31 (m, 4H), 3.31-3.28 (, 4H), 3.17-3.3 (m, 2H), 2.90-2.85 (m, 4H), 2.73-2.62 (m, 7H), 2.55 (t, 2H), 2.26-2.22 (m, 1H), 2.09 (t, 2H), 1.60-1.40 (m, 4H), 1.26-1.15 (m, 26H), 1.00-0.96 (m, 1H), 0.55 (s, 3H), 0.52 (s, 3H), 0.52-4.49 (m, 1H), 0.30-0.21 (m, 2H), 0.10-0.07 (m, 1H).

### Example 17

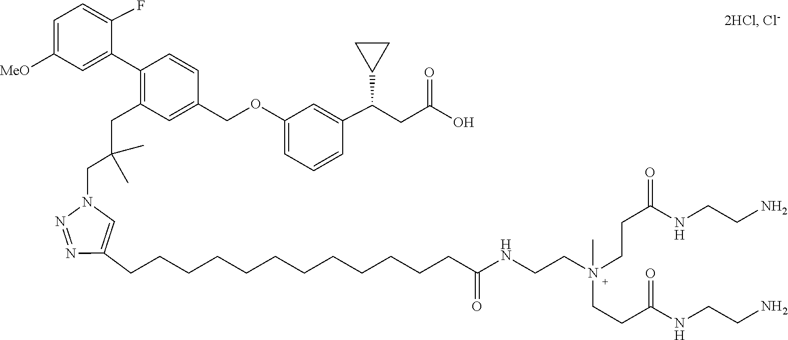

MS (ESI) m/z 1054.5 [M]⁺. ¹H NMR (400 MHZ, DMSO-d₆): δ 8.31 (s, NH, 2H), 8.11 (s, NH, 1H), 7.79 (br, 6H), 7.61 (s, 1H), 7.43 (s, 1H), 7.42-7.39 (d, 1H), 7.22-7.11 (m, 3H), 6.93-6.88 (m, 2H), 6.86-6.75 (m, 3H), 5.13 (s, 2H), 3.97 (s, 2H), 3.73 (s, 3H), 3.59-3.55 (m, 4H), 3.40-3.34 (m, 2H), 3.34-3.23 (m, 6H), 3.02 (s, 3H), 2.90-2.83 (m, 4H), 2.74-2.56 (m, 7H), 2.56-2.44 (m, 3H), 2.26-2.23 (m, 1H), 2.12-2.06 (t, 2H), 1.80-1.70 (m, 4H), 1.26-1.15 (m, 26H), 1.02-0.96 (m, 1H), 0.55 (s, 3H), 0.52 (s, 3H), 0.52-4.49 (m, 1H), 0.32-0.21 (m, 2H), 0.10-0.07 (m, 1H).

### Example 18

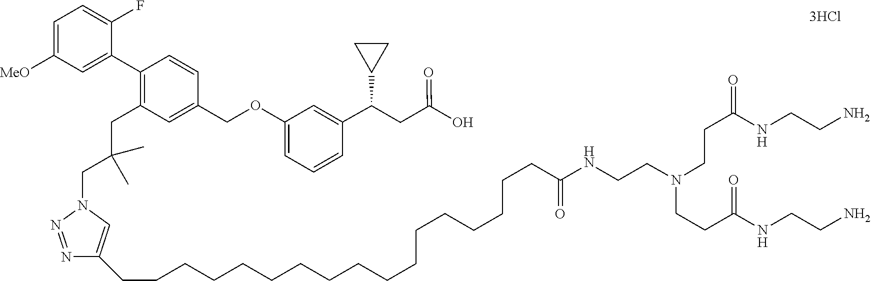 [SMILES: COc1ccc(F)c(-c2ccc(COc3cccc([C@@H](CC(=O)O)C4CC4)c3)cc2CC(C)(C)Cn2cc(CCCCCCCCCCCCCCCCCC(=O)NCCN(CCC(=O)NCCN)CCC(=O)NCCN)nn2)c1]

MS (ESI) m/z 1110.9 [M+H]⁺. ¹H NMR (400 MHZ, DMSO-d₆): δ 10.3 (br, 1H), 8.49 (t, NH, 2H), 8.20 (t, NH, 1H), 8.06 (br, 6H), 7.62 (s, 1H), 7.43 (s, 1H), 7.41-7.39 (m, 1H), 7.23-7.12 (m, 3H), 6.95-6.91 (m, 2H), 6.86-6.77 (m, 3H), 5.14 (s, 2H), 3.98 (s, 2H), 3.73 (s, 3H), 3.48-3.43 (m, 2H), 3.38-3.43 (m, 2H), 3.38-3.35 (m, 4H), 3.32-3.30 (m, 4H), 3.16-3.15 (m, 2H), 2.90-2.86 (m, 4H), 2.72-2.65 (m, 5H), 2.62-2.59 (m, 2H), 2.57-2.53 (m, 3H), 2.26-2.23 (m, 1H), 2.11-2.08 (t, 2H), 1.56-1.46 (m, 4H), 1.27-1.22 (m, 26H), 1.00-0.96 (m, 1H), 0.55 (s, 3H), 0.52 (s, 3H), 0.52-4.49 (m, 1H), 0.30-0.21 (m, 2H), 0.10-0.07 (m, 1H).

### Example 19

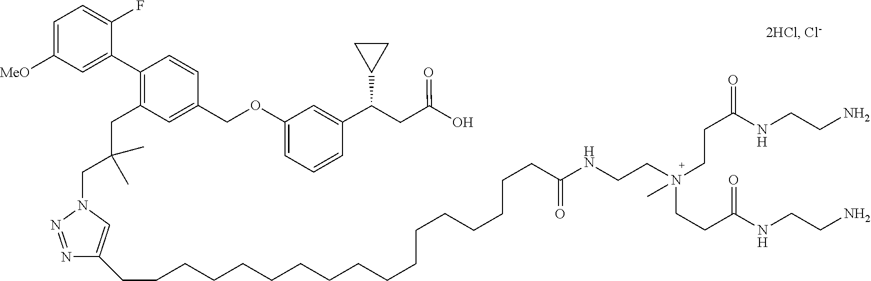

MS (ESI) m/z 1125.0 [M+]⁺. ¹H NMR (400 MHZ, DMSO-d₆): δ=8.60 (t, NH, 2H), 8.30 (t, NH, 1H), 8.17 (br, 6H), 7.64 (s, 1H), 7.44 (s, 1H), 7.41-7.39 (d, 1H), 7.23-7.12 (m, 3H), 6.95-6.91 (m, 2H), 6.86-6.77 (m, 3H), 5.14 (s, 2H), 3.98 (s, 2H), 3.74 (s, 3H), 3.64-3.60 (m, 4H), 3.50-3.44 (m, 2H), 3.37-3.31 (m, 6H), 3.08 (s, 3H), 2.91-2.85 (m, 4H), 2.76-2.70 (m, 5H), 2.66-2.63 (m, 2H), 2.57-2.53 (t, 2H), 2.53-2.52 (m, 1H), 2.27-2.23 (m, 1H), 2.13-2.07 (t, 2H), 1.57-1.43 (m, 4H), 1.33-1.17 (m, 26H), 1.00-0.96 (m, 1H), 0.55 (s, 3H), 0.52 (s, 3H), 0.52-4.49 (m, 1H), 0.30-0.21 (m, 2H), 0.10-0.07 (m, 1H).

### Example 20

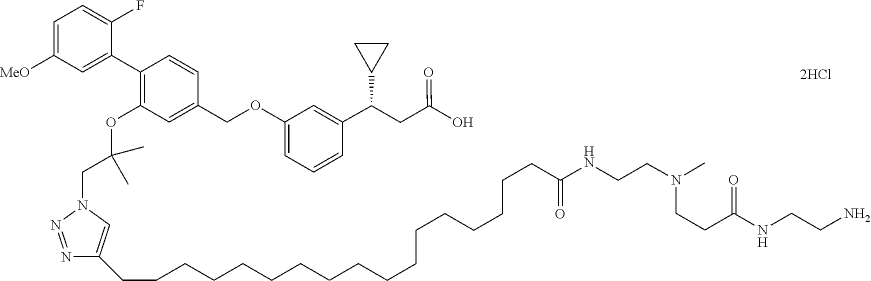 [SMILES: COc1ccc(F)c(-c2ccc(COc3cccc([C@@H](CC(=O)O)C4CC4)c3)cc2OC(C)(C)Cn2cc(CCCCCCCCCCCCCCCCCC(=O)NCCN(C)CCC(=O)NCCN)nn2)c1]

MS (ESI) m/z 1011.4 [M+H]⁺.

### Example 21

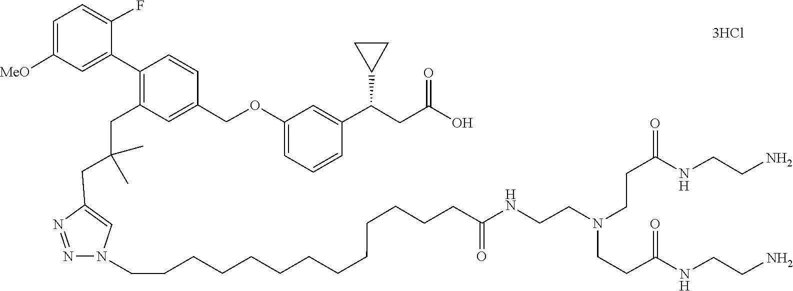 [SMILES: COc1ccc(F)c(-c2ccc(COc3cccc([C@@H](CC(=O)O)C4CC4)c3)cc2CC(C)(C)Cc2cn(CCCCCCCCCCCCCC(=O)NCCN(CCC(=O)NCCN)CCC(=O)NCCN)nn2)c1]

MS (ESI) m/z 527.8 [(M+2H)/2]⁺. ¹H NMR (400 MHZ, DMSO-d₆): δ 10.33 (br, 1H), 8.49 (t, NH, 2H), 8.20 (t, NH, 1H), 8.07 (br, 6H), 7.71 (s, 1H), 7.45 (s, 1H), 7.40-7.37 (d, 1H), 7.22-7.14 (m, 3H), 6.96-6.91 (m, 2H), 6.86-6.77 (m, 3H), 5.12 (s, 2H), 4.25 (t, 2H), 3.73 (s, 3H), 3.45-3.39 (m, 2H), 3.39-3.34 (m, 4H), 3.34-3.31 (m, 4H), 3.18-3.14 (m, 2H), 2.90-2.85 (m, 4H), 2.72-2.66 (m, 5H), 2.66-2.62 (m, 2H), 2.52-2.49 (m, 1H), 2.35 (s, 2H), 2.28-2.24 (m, 1H), 2.11-2.05 (t, 2H), 1.78-1.72 (m, 2H), 1.50-1.45 (m, 2H), 1.26-1.13 (m, 18H), 1.02-0.95 (m, 1H), 0.53-0.50 (br, 6H), 0.51-0.47 (m, 1H), 0.31-0.21 (m, 2H), 0.11-0.07 (m, 1H).

### Example 22

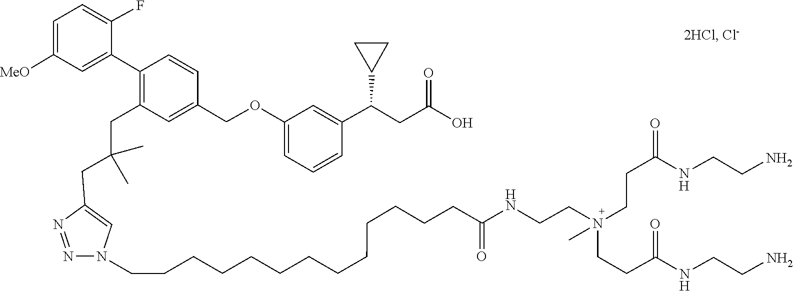

MS (ESI) m/z 534.8 [(M+H)/2]⁺. ¹H NMR (400 MHZ, DMSO-d₆): δ 8.59 (t, NH, 2H), 8.31 (t, NH, 1H), 8.17 (br, 6H), 7.71 (s, 1H), 7.44 (s, 1H), 7.39-7.37 (d, 1H), 7.22-7.14 (m, 3H), 6.96-6.91 (m, 2H), 6.86-6.77 (m, 3H), 5.12 (s, 2H), 4.25 (t, 2H), 3.74 (s, 3H), 3.65-3.59 (m, 4H), 3.55-3.50 (m, 2H), 3.38-3.31 (m, 6H), 3.08 (s, 3H), 2.91-2.86 (m, 4H), 2.76-2.65 (m, 5H), 2.63-2.58 (m, 2H), 2.54-2.49 (m, 1H), 2.35 (s, 2H), 2.28-2.24 (m, 1H), 2.12-2.08 (t, 2H), 1.78-1.72 (m, 2H), 1.51-1.45 (m, 2H), 1.26-1.13 (m, 18H), 1.02-0.95 (m, 1H), 0.55-0.50 (br, 6H), 0.51-0.47 (m, 1H), 0.31-0.21 (m, 2H), 0.11-0.07 (m, 1H).

### Example 23

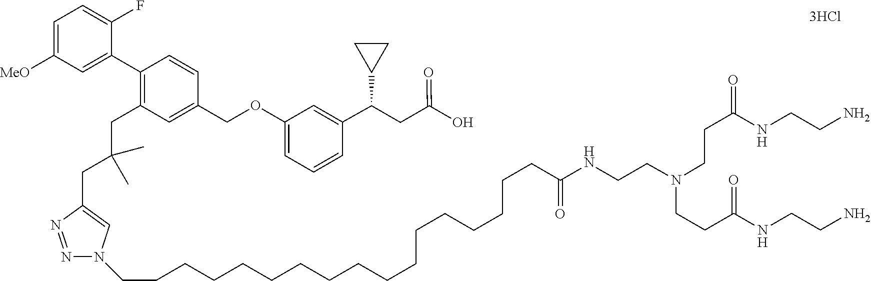 [SMILES: COc1ccc(F)c(-c2ccc(COc3cccc([C@@H](CC(=O)O)C4CC4)c3)cc2CC(C)(C)Cc2cn(CCCCCCCCCCCCCCCCCC(=O)NCCN(CCC(=O)NCCN)CCC(=O)NCCN)nn2)c1]

MS (ESI) m/z 1110.5 [M+H]⁺. ¹H NMR (400 MHZ, DMSO-d₆): δ 10.30 (br, 1H), 8.49 (t, NH, 2H), 8.21 (t, NH, 1H), 8.05 (br, 6H), 7.72 (s, 1H), 7.45 (s, 1H), 7.40-7.38 (d, 1H), 7.22-7.14 (m, 3H), 6.92-6.88 (m, 2H), 6.86-6.78 (m, 3H), 5.12 (s, 2H), 4.28 (t, 2H), 3.74 (s, 3H), 3.51-3.44 (m, 2H), 3.40-3.33 (m, 4H), 3.33-3.28 (m, 4H), 3.18-3.14 (m, 2H), 2.91-2.85 (m, 4H), 2.69-2.63 (m, 5H), 2.63-2.58 (m, 2H), 2.51-2.47 (m, 1H), 2.35 (s, 2H), 2.27-2.22 (m, 1H), 2.11-2.06 (t, 2H), 1.78-1.72 (m, 2H), 1.50-1.45 (m, 2H), 1.26-1.13 (m, 26H), 1.02-0.95 (m, 1H), 0.53-0.50 (br, 6H), 0.51-0.46 (m, 1H), 0.31-0.21 (m, 2H), 0.11-0.07 (m, 1H)

### Example 24

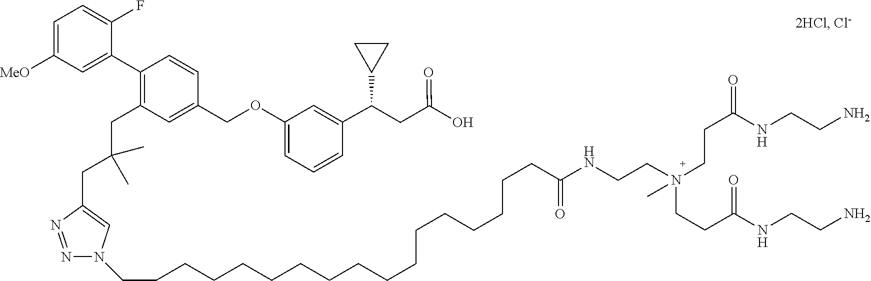

MS (ESI) m/z 1125.4 [M]⁺. ¹H NMR (400 MHZ, DMSO-d₆): δ 8.56 (t, NH, 2H), 8.26 (t, NH, 1H), 8.09 (br, 6H), 7.71 (s, 1H), 7.45 (s, 1H), 7.39-3.37 (d, 1H), 7.22-7.14 (m, 3H), 6.95-6.91 (m, 2H), 6.85-6.78 (m, 3H), 5.12 (s, 2H), 4.25 (t, 2H), 3.74 (s, 3H), 3.62-3.59 (m, 4H), 3.49-3.44 (m, 2H), 3.35-3.30 (m, 6H), 3.07 (s, 3H), 2.90-2.84 (m, 4H), 2.75-2.66 (m, 5H), 2.66-2.60 (m, 2H), 2.51-2.49 (m, 1H), 2.34 (s, 2H), 2.27-2.22 (m, 1H), 2.11-2.06 (t, 2H), 1.76-1.72 (m, 2H), 1.50-1.45 (m, 2H), 1.26-1.13 (m, 26H), 1.01-0.95 (m, 1H), 0.55-0.50 (br, 6H), 0.51-0.46 (m, 1H), 0.31-0.21 (m, 2H), 0.11-0.07 (m, 1H).

### Example 25

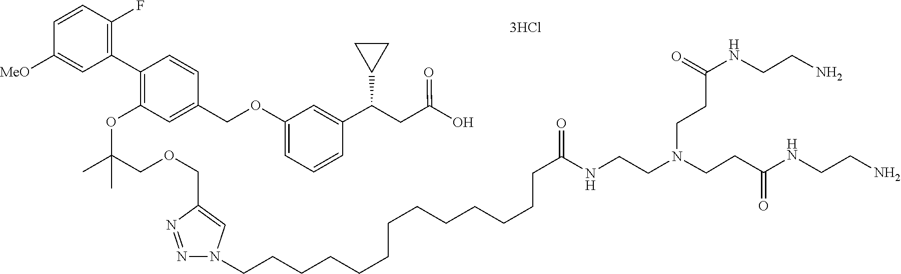 [SMILES: COc1ccc(F)c(-c2ccc(COc3cccc([C@@H](CC(=O)O)C4CC4)c3)cc2OC(C)(C)COCc2cn(CCCCCCCCCCCCCC(=O)NCCN(CCC(=O)NCCN)CCC(=O)NCCN)nn2)c1]

MS (ESI) m/z 1086.4 [M+H]⁺. ¹H NMR (400 MHZ, DMSO-d₆): δ 10.37 (br, 1H), 8.51 (t, NH, 2H), 8.23 (t, NH, 1H), 8.13 (br, 6H), 7.97 (s, 1H), 7.29-7.27 (d, 1H), 7.25 (s, 1H), 7.21-7.11 (m, 3H), 6.93-6.82 (m, 5H), 5.10 (s, 2H), 4.44 (s, 2H), 4.27 (t, 2H), 3.73 (s, 3H), 3.50-3.44 (m, 2H), 3.41-3.37 (m, 4H), 3.37-3.28 (m, 4H), 3.27 (s, 2H), 3.18-3.14 (m, 2H), 2.90-2.84 (m, 4H), 2.72-2.66 (m, 4H), 2.66-2.58 (m, 2H), 2.27-2.22 (m, 1H), 2.10-2.06 (t, 2H), 1.77-1.73 (m, 2H), 1.50-1.46 (m, 2H), 1.27-1.11 (m, 18H), 1.06-0.94 (m, 1H), 0.96 (s, 6H), 0.50-0.45 (m, 1H), 0.28-0.16 (m, 2H), 0.12-0.06 (m, 1H).

### Example 26

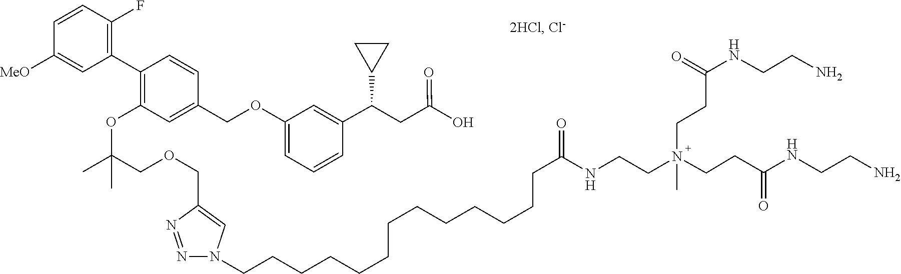

MS (ESI) m/z 1100.4 [M]⁺. ¹H NMR (400 MHZ, DMSO-d₆): δ 8.60 (t, NH, 2H), 8.31 (t, NH, 1H), 8.16 (br, 6H), 7.99 (s, 1H), 7.31-7.29 (d, 1H), 7.26 (s, 1H), 7.23-7.13 (m, 3H), 6.94-6.84 (m, 5H), 5.11 (s, 2H), 4.46 (s, 2H), 4.29 (t, 2H), 3.73 (s, 3H), 3.68-3.61 (m 4H), 3.38-3.21 (m, 6H), 3.28 (s, 2H), 3.07 (s, 3H), 2.91-2.84 (m, 4H), 2.75-2.66 (m, 4H), 2.66-2.59 (m, 2H), 2.28-2.22 (m, 1H), 2.12-2.08 (t, 2H), 1.77-1.73 (m, 2H), 1.51-1.46 (m, 2H), 1.27-1.11 (m, 18H), 1.06-0.94 (m, 1H), 0.96 (s, 6H), 0.51-0.45 (m, 1H), 0.30-0.16 (m, 2H), 0.12-0.06 (m, 1H).

### Example 27

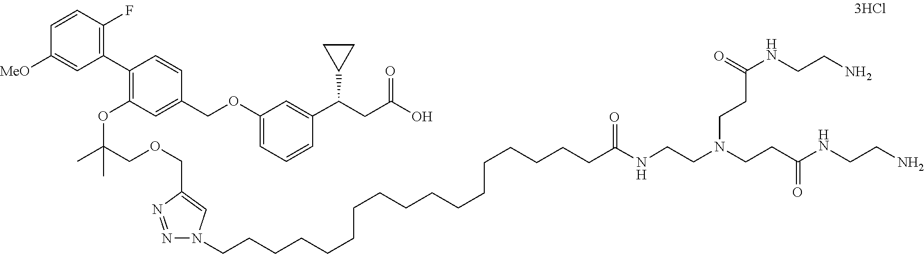 [SMILES: COc1ccc(F)c(-c2ccc(COc3cccc([C@@H](CC(=O)O)C4CC4)c3)cc2OC(C)(C)COCc2cn(CCCCCCCCCCCCCCCCCC(=O)NCCN(CCC(=O)NCCN)CCC(=O)NCCN)nn2)c1]

MS (ESI) m/z 1142.5 [M+H]⁺. ¹H NMR (400 MHZ, DMSO-d₆): δ 10.33 (br, 1H), 8.50 (t, NH, 2H), 8.2 8.25-8.13 (t, NH, 1H), 8.09 (br, 6H), 7.99 (s, 1H), 7.23-7.12 (m, 5H), 6.93-6.84 (m, 5H), 5.11 (s, 2H), 4.46 (s, 2H), 4.29 (t, 2H), 3.73 (s, 3H), 3.47-3.41 (m, 2H), 3.41-3.34 (m, 4H), 3.34-3.28 (m, 4H), 3.27 (s, 2H), 3.15-3.10 (m, 2H), 2.91-2.85 (m, 4H), 2.73-2.63 (m, 6H), 2.26-2.21 (m, 1H), 2.11-2.07 (t, 2H), 1.77-1.73 (m, 2H), 1.51-1.46 (m, 2H), 1.28-1.11 (m, 26H), 1.06-0.94 (m, 1H), 0.96 (s, 6H), 0.51-0.45 (m, 1H), 0.30-0.16 (m, 2H), 0.12-0.06 (m, 1H).

### Example 28

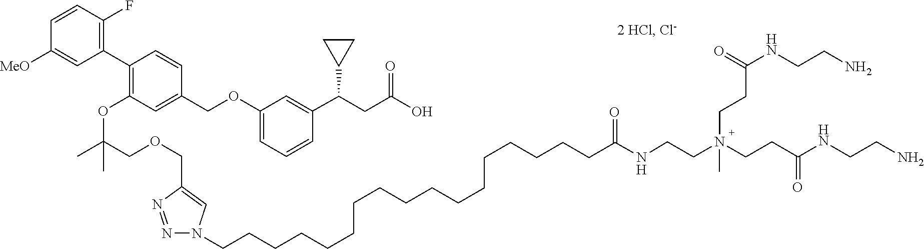

MS (ESI) m/z 1156.6 [M+]⁺. ¹H NMR (400 MHZ, DMSO-d₆): δ 8.61 (t, NH, 2H), 8.34 (t, NH, 1H), 8.21 (br, 6H), 7.98 (s, 1H), 7.29-7.27 (d, 1H), 7.25 (s, 1H), 7.22-7.11 (m, 3H), 6.92-6.81 (m, 5H), 5.10 (s, 2H), 4.45 (s, 2H), 4.28 (t, 2H), 3.72 (s, 3H), 6.66-3.59 (m, 4H), 3.52-3.41 (m, 2H), 3.38-3.31 (m, 6H), 3.27 (s, 2H), 3.07 (s, 3H), 2.90-2.85 (m, 4H), 2.73-2.68 (t, 4H), 2.63-2.57 (m, 2H), 2.26-2.21 (m, 1H), 2.11-2.07 (t, 2H), 1.77-1.73 (m, 2H), 1.50-1.46 (m, 2H), 1.27-1.11 (m, 26H), 1.06-0.94 (m, 1H), 0.96 (s, 6H), 0.50-0.45 (m, 1H), 0.28-0.16 (m, 2H), 0.12-0.06 (m, 1H).

### Example 29

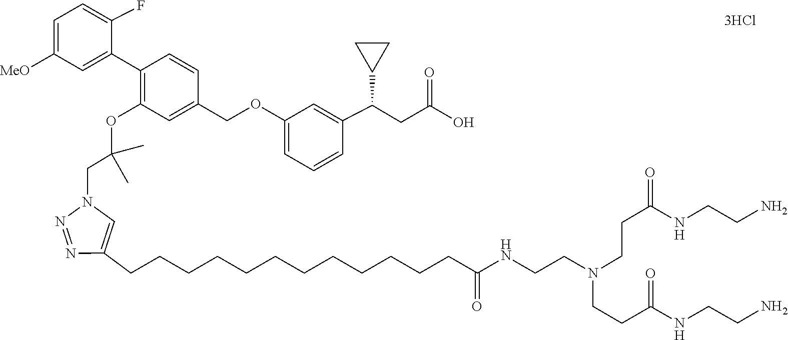 [SMILES: COc1ccc(F)c(-c2ccc(COc3cccc([C@@H](CC(=O)O)C4CC4)c3)cc2OC(C)(C)Cn2cc(CCCCCCCCCCCCC(=O)NCCN(CCC(=O)NCCN)CCC(=O)NCCN)nn2)c1]

MS (ESI) m/z 1042.4 [M+H]⁺. ¹H NMR (400 MHZ, DMSO-d₆): δ 10.07 (br, 1H), 8.46 (t, NH, 2H), 8.45 (t, NH, 1H), 7.97 (br, 6H), 7.32-7.30 (d, 1H), 7.24-7.17 (m, 4H), 7.11 (s, 1H), 6.97-6.82 (m, 5H), 5.13 (s, 2H), 4.30 (s, 2H), 3.75 (s, 3H), 3.5-3.3 (m, 6H), 3.3-3.25 (m, 4H), 3.31-3.25 (m, 2H), 2.91-2.85 (m, 4H), 2.69-2.61 (m, 6H), 2.56-2.50 (t, 2H), 2.2-2.21 (m, 1H), 2.09 (t, 2H), 1.53-1.45 (m, 4H), 1.3-1.18 (m, 26H), 1.06 (s, 6H), 1.01-0.96 (m, 1H), 0.51-0.43 (m, 1H), 0.30-0.20 (m, 2H), 0.10-0.07 (m, 1H).

### Example 30

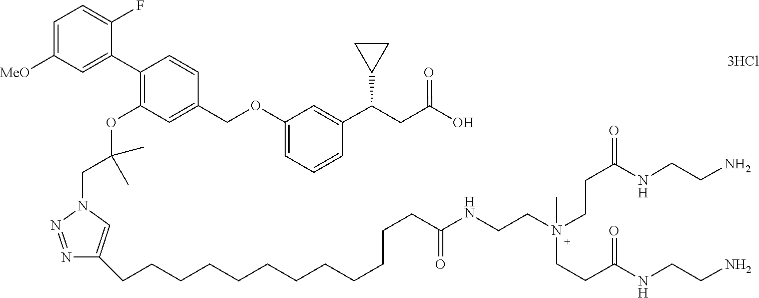

MS (ESI) m/z 1057.3 [M+H]⁺. ¹H NMR (400 MHz, DMSO-d₆): δ 8.32 (t, NH, 2H), 8.37 (t, NH, 1H), 7.79 (br, 6H), 7.31-7.29 (d, 1H), 7.20-7.15 (m, 4H), 7.10 (s, 1H), 6.94-6.90 (m, 1H), 6.89-6.84 (m, 2H), 6.83-6.80 (m, 2H), 5.11 (s, 2H), 4.29 (s, 2H), 3.74 (s, 3H), 3.58-3.54 (t, 4H), 3.44-3.31 (m, 2H), 3.31-3.25 (m, 6H), 3.01 (s, 3H), 2.89-2.81 (m, 4H), 2.66-2.58 (m, 6H), 2.55-2.48 (t, 2H), 2.25-2.16 (m, 1H), 2.09 (t, 2H), 1.53-1.45 (m, 4H), 1.3-1.18 (m, 26H), 1.06 (s, 6H), 1.01-0.96 (m, 1H), 0.50-0.43 (m, 1H), 0.30-0.20 (m, 2H), 0.10-0.07 (m, 1H).

### Example 31

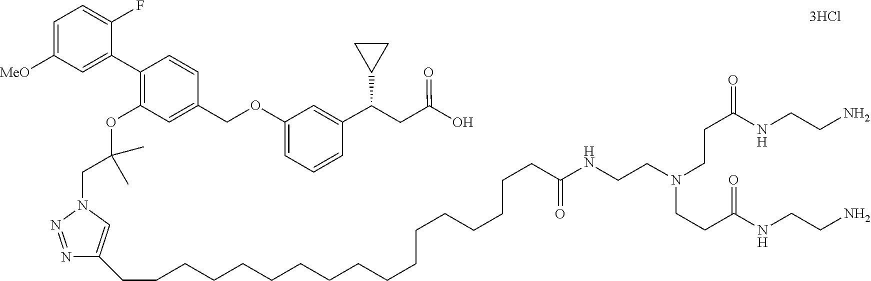 [SMILES: COc1ccc(F)c(-c2ccc(COc3cccc([C@@H](CC(=O)O)C4CC4)c3)cc2OC(C)(C)Cn2cc(CCCCCCCCCCCCCCCCCC(=O)NCCN(CCC(=O)NCCN)CCC(=O)NCCN)nn2)c1]

MS (ESI) m/z 1112.5 [M+H]⁺. ¹H NMR (400 MHZ, DMSO-d₆): δ 10.36 (br, 1H), 8.51 (t, NH, 2H), 8.23 (t, NH, 1H), 8.10 (br, 6H), 7,32-7.30 (d, 1H), 7.24-7.16 (m, 4H), 7.12 (s, 1H), 6.97-6.94 (, m, 1H), 6.91-6.83 (m, 4H), 5.13 (s, 2H), 4.31 (s, 2H), 3.76 (s, 3H), 3.57-3.44 (m, 2H), 3.44-3.37 (m, 4H), 3.37-3.31 (m, 4H), 3.17-3.12 (m, 2H), 2.93-2.81 (m, 4H), 2.72-2.68 (t, 4H), 2.66-2.58 (m, 2H), 2.55-2.50 (t, 2H), 2.26-2.23 (m, 1H), 2.10 (t, 2H), 1.52-1.43 (m, 4H), 1.3-1.18 (m, 26H), 1.06 (s, 6H), 1.01-0.96 (m, 1H), 0.52-0.43 (m, 1H), 0.30-0.20 (m, 2H), 0.10-0.07 (m, 1H).

### Example 32

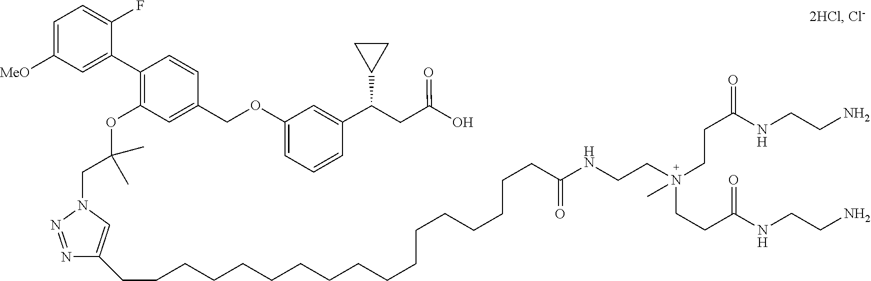

MS (ESI) m/z 1126.6 [M]⁺. ¹H NMR (400 MHZ, DMSO-d₆): δ 8.58 (t, NH, 2H), 8.28 (t, NH, 1H), 8.11 (br, 6H), 7.32-7.30 (d, 1H), 7.23-7.16 (m, 4H), 7.11 (s, 1H), 6.97-6.93 (m, 1H), 6.92-6.88 (m 2H), 6.86-6.83 (m, 2H), 5.13 (s, 2H), 4.31 (s, 2H), 3.76 (s, 3H), 3.64-3.59 (t, 4H), 3.5-3.4 (m, 2H), 3.38-3.28 (m, 6H), 3.07 (s, 3H), 2.90-2.85 (m, 4H), 2.73-2.67 (t, 4H), 2.66-2.60 (m, 2H), 2.55-2.49 (t, 2H), 2.25-2.21 (m, 1H), 2.11-2.08 (t, 2H), 1.52-1.43 (m, 4H), 1.3-1.18 (m, 26H), 1.06 (s, 6H), 1.01-0.96 (m, 1H), 0.51-0.44 (m, 1H), 0.30-0.20 (m, 2H), 0.10-0.07 (m, 1H).

### Example 33

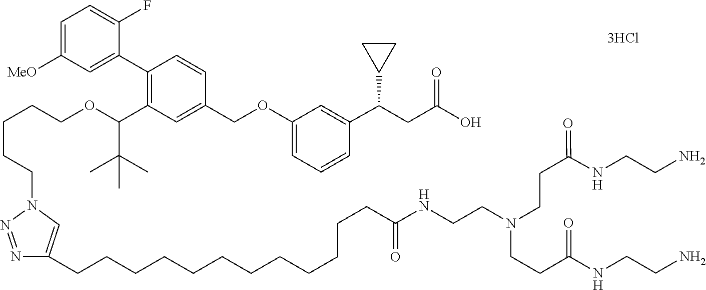 [SMILES: COc1ccc(F)c(-c2ccc(COc3cccc([C@@H](CC(=O)O)C4CC4)c3)cc2C(OCCCCCn2cc(CCCCCCCCCCCCC(=O)NCCN(CCC(=O)NCCN)CCC(=O)NCCN)nn2)C(C)(C)C)c1]

MS (ESI) m/z 1126.5 [M+H]⁺. ¹H NMR (400 MHZ, DMSO-d₆): δ 10.30 (br, 1H), 8.48 (t, NH, 2H), 8.19 (t, NH, 1H), 8.03 (br, 6H), 7.78 (s, 1H), 7.54-7.43 (m, 2H), 7.22-16 (m, 3H), 6.99-6.78 (m, 5H), 5.17 (s, 2H), 4.29-4.26 (t, 2H), 4.17, 3.93 (s, s, rotamers, 1H). 3.74 (s, 3H), 3.49-3.46 (m, 2H), 3.39-3.32 (m, 10H), 3.17-3.13 (m, 2H), 2.90-2.87 (m, 4H), 2.69-2.63 (t, 4H), 2.62-2.56 (m, 2H), 2.56-2.50 (t, 2H), 2.26-2.20 (m, 1H), 2.10 (t, 2H), 1.81-1.69 (m, 2H), 1.56-1.40 (m, 6H), 1.29-1.19 (m, 16H), 1.01-0.94 (m, 1H), 0.61 (s, 9H), 0.49-0.44 (m, 1H), 0.28-0.19 (m, 2H), 0.09-0.04 (m, 1H).

### Example 34

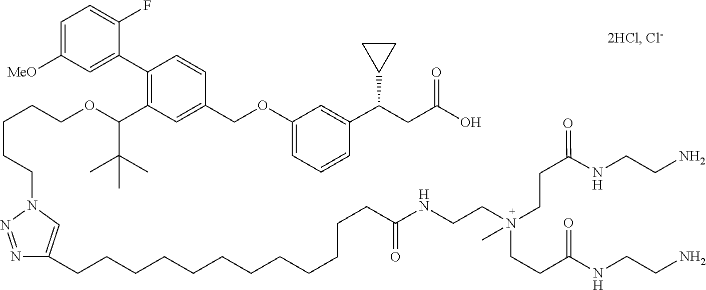

MS (ESI) m/z 1141.4 [M+H]⁺. ¹H NMR (400 MHZ, DMSO-d₆): δ 8.54 (t, NH, 2H), 8.25 (t, NH, 1H), 8.07 (brs, 6H), 7.78 (s, 1H), 7.54-7.50 (d, 1H), 7.50-7.45 (m, 1H), 7.44-7.38 (m, 3H), 6.99-6.94 (m, 1H), 6.90 (s, 1H), 6.88-6.70 (m, 3H), 5.17 (s, 2H), 4.29-4.26 (t, 2H), 4.17, 3.94 (s, s, 1H), 3.74 (s, 3H), 3.64-3.60 (m, 4H), 3.47-3.42 (m, 2H), 3.37-3.31 (m, 6H), 3.30-3.13 (m, 2H), 3.07 (s, 3H), 2.91-2.84 (m, 4H), 2.72-2.65 (m, 4H), 2.63-2.53 (m, 4H), 2.25-2.20 (m, 1H), 2.11-2.09 (t, 2H), 1.80-1.74 (m, 2H), 1.57-1.42 (m, 6H), 1.30-1.16 (m, 18H), 0.99-0.92 (m, 1H), 0.60 (s, 9H), 0.49-0.42 (m, 1H), 0.26-0.18 (m, 2H), 0.07-0.02 (m, 1H).

### Example 35

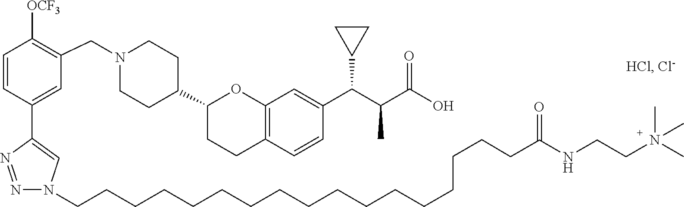

MS (ESI) m/z 476.3 [(M+H)/2]⁺. ¹H NMR (400 MHZ, DMSO-d₆): δ 8.67 (s, 1H), 8.56 (s, 1H), 8.33 (t, NH, 1H), 8.05-8.02 (d, 1H), 7.58-7.56 (d, 1H), 6.96-6.94 (d, 1H), 6.65-6.63 (d, 1H), 6.51 (s, 1H), 4.44-4.40 (m, 4H), 3.80-3.77 (m, 1H), 3.55-3.20 (m, 8H), 3.10 (s, 9H), 2.77-2.57 (m, 3H), 2.13-2.03 (m, 3H), 2.00-1.70 (m, 7H), 1.69-1.56 (m, 2H), 1.56-1.39 (m, 3H), 1.36-1.14 (m, 26H), 1.10-1.01 (m, 1H), 0.81-0.79 (d, 3H), 0.54-0.46 (m, 2H), -0.09 ˜−0.17 (m, 1H).

### Example 36

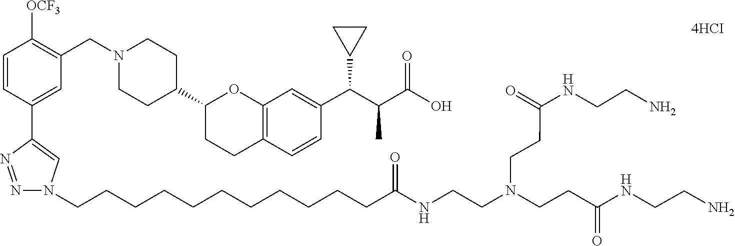 [SMILES: C[C@H](C(=O)O)[C@H](c1ccc2c(c1)O[C@@H](C1CCN(Cc3cc(-c4cn(CCCCCCCCCCCC(=O)NCCN(CCC(=O)NCCN)CCC(=O)NCCN)nn4)ccc3OC(F)(F)F)CC1)CC2)C1CC1]

MS (ESI) m/z 1153.4 [M+H]⁺. ¹H NMR (400 MHZ, DMSO-d₆) δ: 9.88 (brs, 1H), 8.66 (s, 1H), 8.40-8.37 (m, 2H), 8.25 (t, NH, 1H), 8.02-7.99 (d, 1H), 7.90 (br, 6H), 7.60-7.58 (d, 1H), 6.97-6.95 (d, 1H), 6.66-6.64 (d, 1H), 6.51 (s, 1H), 4.44-4.40 (m, 4H), 3.82-3.80 (m, 1H), 339-3.29 (m, 12H), 3.16-3.11 (m, 4H), 2.88 (m, 4H), 2.77-2.60 (m, 7H), 2.10 (t, 3H), 1.97-1.76 (m, 8H), 1.74-1.65 (m, 2H), 1.50-1.46 (m, 2H), 1.27-1.22 (m, 14H), 1.09-1.03 (m, 1H), 0.80 (d, 3H), 0.52-0.49 (m, 1H), 0.25-0.23 (m, 2H), −0.08 ˜−0.12 (m, 1H).

### Example 37

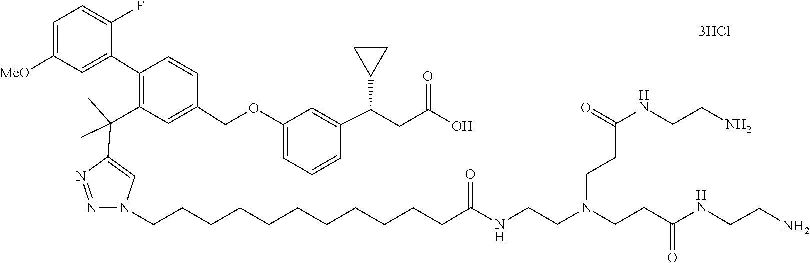 [SMILES: COc1ccc(F)c(-c2ccc(COc3cccc([C@@H](CC(=O)O)C4CC4)c3)cc2C(C)(C)c2cn(CCCCCCCCCCCC(=O)NCCN(CCC(=O)NCCN)CCC(=O)NCCN)nn2)c1]

MS (ESI) m/z 998.3 [M+H]⁺. ¹H NMR (400 MHZ, DMSO-d₆) δ: 9.90 (br, 1H), 8.39 (t, NH, 2H), 8.13 (t, NH, 1H), 7.87 (brs, —NH₃, 6H), 7.69 (s, 1H), 7.35 (d, 1H), 7.31 (s, 1H), 7.22 (t, 1H), 7.01-6.89 (m, 5H), 6.87-6.78 (m, 1H), 5.95-5.92 (m, 1H), 5.14 (s, 2H), 4.11-4.05 (m, 2H), 3.60 (s, 3H), 3.43-3.37 (m, 6H), 3.34-3.29 (m, 4H), 3.19-3.15 (m, 2H), 2.91-2.86 (m, 4H), 2.70-2.62 (m, 6H), 2.29-2.25 (m, 1H), 2.11-2.07 (m, 2H), 1.75-1.70 (m, 2H), 1.60 (s, 3H), 1.50-1.47 (m, 5H), 1.35-1.15 (m, 14H), 1.05-1.00 (m, 1H), 0.53-0.47 (m, 1H), 0.34-0.23 (m, 2H), 0.15-0.10 (m, 1H).

### Example 38

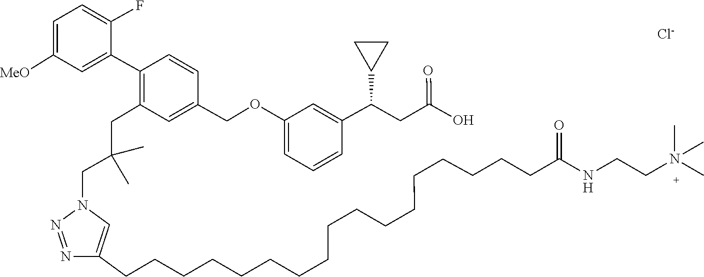

MS (ESI) m/z 462.8 [(M+H)/2]⁺. ¹H NMR (400 MHZ, DMSO-d₆): δ 8.33 (t, NH, 1H), 7.64 (s, 1H), 7.43 (s, 1H), 7.41-7.38 (d, 1H), 7.23-7.11 (m, 3H), 6.94-6.91 (m, 2H), 6.85-6.76 (m, 3H), 5.13 (s, 2H), 4.16 (s, 2H), 3.73 (s, 3H), 3.48-3.43 (m, 2H), 3.40-3.34 (m, 2H), 3.10 (s, 9H), 2.74-2.60 (m, 3H), 2.60-2.50 (m, 3H), 2.28-2.23 (m, 1H), 2.11-2.05 (t, 2H), 1.60-1.40 (m, 4H), 1.34-1.16 (m, 26H), 1.02-0.97 (m, 1H), 0.55 (s, 3H), 0.52 (s, 3H), 0.51-0.47 (m, 1H), 0.30-0.17 (m, 2H), 0.12-0.07 (m, 1H).

### Example 39

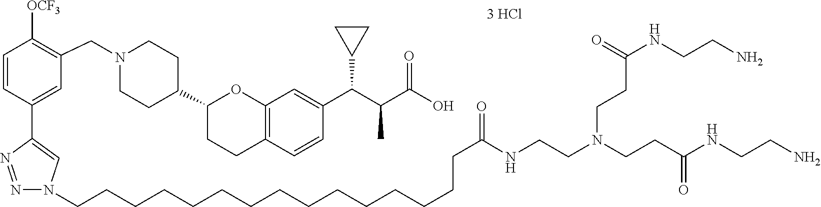 [SMILES: C[C@H](C(=O)O)[C@H](c1ccc2c(c1)O[C@@H](C1CCN(Cc3cc(-c4cn(CCCCCCCCCCCCCCCC(=O)NCCN(CCC(=O)NCCN)CCC(=O)NCCN)nn4)ccc3OC(F)(F)F)CC1)CC2)C1CC1]

MS (ESI) m/z 1109.4 [M+H]⁺. ¹H NMR (400 MHZ, DMSO-d₆) ¿: 9.88 (brs, 1H), 8.67 (s, 1H), 8.45-8.40 (m, 3H), 8.23 (t, NH, 1H), 8.02-7.99 (d, 1H), 7.99-7.90 (br, 6H), 7.61-7.56 (d, 1H), 6.97-6.95 (d, 1H), 6.66-6.64 (d, 1H), 6.52 (s, 1H), 4.44-4.40 (m, 4H), 3.82-3.80 (m, 1H), 3.39-3.29 (m, 12H), 3.16-3.11 (m, 4H), 2.89-2.87 (m, 4H), 2.77-2.60 (m, 7H), 2.11-2.06 (t, 3H), 2.00-1.78 (m, 8H), 1.76-1.65 (m, 2H), 1.52-1.47 (m, 2H), 1.27-1.22 (m, 22H), 1.09-1.03 (m, 1H), 0.80 (d, 3H), 0.52-0.49 (m, 1H), 0.25-0.23 (m, 2H), −0.08 ˜−0.12 (m, 1H).

### Example 40

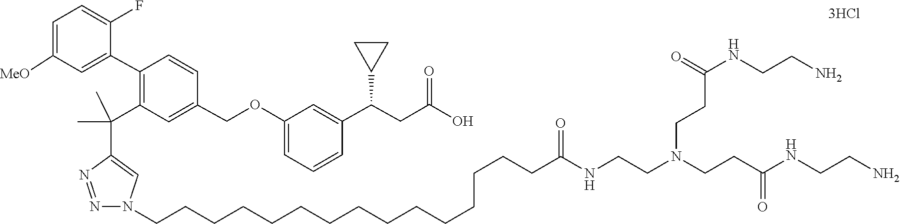 [SMILES: COc1ccc(F)c(-c2ccc(COc3cccc([C@@H](CC(=O)O)C4CC4)c3)cc2C(C)(C)c2cn(CCCCCCCCCCCCCCCC(=O)NCCN(CCC(=O)NCCN)CCC(=O)NCCN)nn2)c1]

MS (ESI) m/z 1054.8 [M+H]⁺. ¹H NMR (400 MHZ, DMSO-d₆) δ: 10.30 (br, 1H), 8.50 (t, NH, 2H), 8.17 (t, NH, 1H), 8.08 (brs, —NH₃⁺, 6H), 7.65 (s, 1H), 7.35 (d, 1H), 7.31 (s, 1H), 7.22 (t, 1H), 7.00-6.78 (m, 7H), 5.13 (s, 2H), 4.11-4.05 (m, 2H), 3.60 (s, 3H), 3.43-3.37 (m, 6H), 3.34-3.29 (m, 4H), 3.19-3.15 (m, 2H), 2.91-2.86 (m, 4H), 2.70-2.62 (m, 6H), 2.29-2.25 (m, 1H), 2.11-2.07 (m, 2H), 1.75-1.70 (m, 2H), 1.60 (s, 3H), 1.50-1.47 (m, 5H), 1.35-1.15 (m, 14H), 1.05-1.00 (m, 1H), 0.53-0.47 (m, 1H), 0.34-0.23 (m, 2H), 0.15-0.10 (m, 1H).

### Example 41

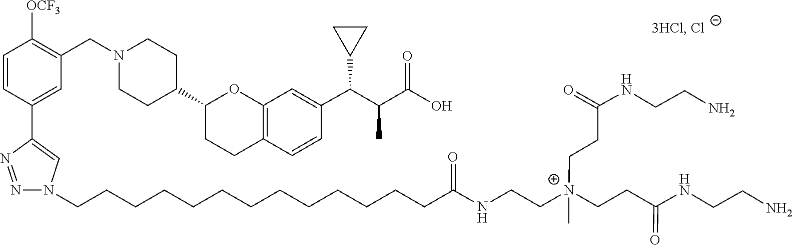 [SMILES: C[C@H](C(=O)O)[C@H](c1ccc2c(c1)O[C@@H](C1CCN(Cc3cc(-c4cn(CCCCCCCCCCCCCC(=O)NCC[N+](C)(CCC(=O)NCCN)CCC(=O)NCCN)nn4)ccc3OC(F)(F)F)CC1)CC2)C1CC1]

MS (ESI) m/z 1195.5 [M]⁺. ¹H NMR (400 MHz, DMSO-d₆) δ: 10.41 (br, 1H), 8.67 (s, 1H), 8.40-8.57 (t, 2H), 8.51 (s, 1H), 8.28 (t, 1H), 8.12 (brs, 6H), 8.04-8.01 (d, 1H), 7.59-7.57 (d, 1H), 6.97-6.95 (d, 1H), 6.66-6.64 (d, 1H), 6.52 (s, 1H), 4.44-4.41 (m, 4H), 3.81-3.79 (m, 1H), 3.63-3.60 (m, 4H), 3.50-3.44 (m, 2H), 3.44-3.17 (m, 8H), 3.16-3.07 (m, 2H), 3.07 (s, 3H), 2.90-2.87 (m, 4H), 2.85-2.60 (m, 7H), 2.11-2.07 (m, 3H), 2.06-1.57 (m, 9H), 1.52-1.44 (m, 2H), 1.30-1.18 (m, 18H), 1.06-0.98 (m, 1H), 0.80 (d, 3H), 0.52-0.47 (m, 1H), 0.26-0.19 (m, 2H), −0.09−−0.10 (m, 1H).

### Example 42

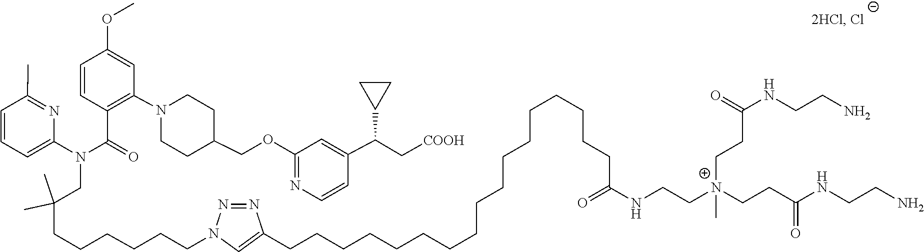 [SMILES: COc1ccc(C(=O)N(CC(C)(C)CCCCCCn2cc(CCCCCCCCCCCCCCCCCC(=O)NCC[N+](C)(CCC(=O)NCCN)CCC(=O)NCCN)nn2)c2cccc(C)n2)c(N2CCC(COc3cc([C@@H](CC(=O)O)C4CC4)ccn3)CC2)c1]

MS (ESI) m/z 1318.7 [M]⁺. ¹H NMR (400 MHZ, DMSO-d₆): δ 8.62 (t, NH, 2H), 8.34 (t, NH, 1H), 8.20 (br, 6H), 8.11-8.10 (d, 1H), 7.88 (s, 1H), 7.38-7.34 (t, 1H), 7.13-7.11 (d, 1H), 7.05 (br, 1H), 6.91 (br, 2H), 6.56-6.53 (m, 2H), 6.33 (s, 1H), 4.27-4.25 (m, 2H), 4.19-4.17 (d, 2H), 4.08-4.04 (br, 2H), 3.70 (s, 3H), 3.64-3.59 (m, 4H), 3.50-3.45 (m, 2H), 3.37-3.30 (m, 6H), 3.08 (s, 3H), 2.90-2.85 (m, 4H), 2.75-2.68 (m, 6H), 2.60-2.55 (t, 2H), 2.36 (s, 3H), 2.32-2.28 (m, 1H), 2.11-2.08 (t, 2H), 1.82-1.75 (m, 1H), 1.73-1.65 (m, 4H), 1.60-1.35 (m, 7H), 1.30-1.15 (m, 26H), 1.12-0.90 (m, 9H), 0.75 (s, 6H), 0.55-0.50 (m, 1H), 0.34-0.29 (m, 2H), 0.21-0.18 (m, 1H).

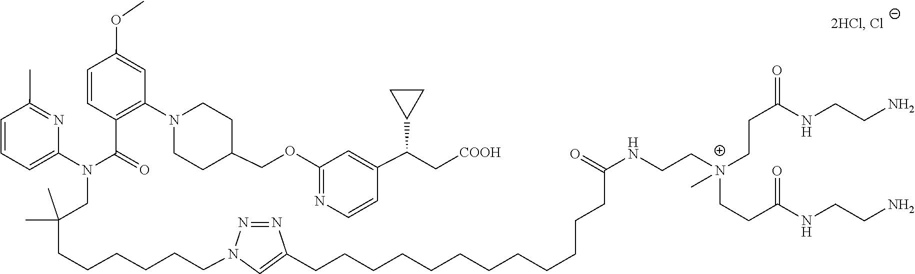 [SMILES: COc1ccc(C(=O)N(CC(C)(C)CCCCCCn2cc(CCCCCCCCCCCCC(=O)NCC[N+](C)(CCC(=O)NCCN)CCC(=O)NCCN)nn2)c2cccc(C)n2)c(N2CCC(COc3cc([C@@H](CC(=O)O)C4CC4)ccn3)CC2)c1]

MS (ESI) m/z 1248.6 [M]⁺. ¹H NMR (400 MHZ, DMSO-d₆): δ 8.61 (t, NH, 2H), 8.31 (t, NH, 1H), 8.19 (br, 6H), 8.11-8.10 (d, 1H), 7.87 (s, 1H), 7.36-7.35 (t, 1H), 7.13-7.11 (d, 1H), 7.04 (br, 1H), 6.91-6.89 (br, 2H), 6.56-6.53 (m, 2H), 6.32 (s, 1H), 4.26-4.25 (m, 2H), 4.19-4.17 (d, 2H), 4.08 (br, 2H), 3.70 (s, 3H), 3.64-3.59 (m, 4H), 3.50-3.45 (m, 2H), 3.37-3.31 (m, 6H), 3.08 (s, 3H), 2.90-2.85 (m, 4H), 2.75-2.68 (m, 6H), 2.60-2.55 (t, 2H), 2.36 (s, 3H), 2.32-2.28 (m, 1H), 2.11-2.08 (t, 2H), 1.82-1.75 (m, 1H), 1.73-1.68 (m, 4H), 1.60-1.35 (m, 7H), 1.30-1.15 (m, 26H), 1.12-0.90 (m, 9H), 0.75 (s, 6H), 0.55-0.50 (m, 1H), 0.34-0.29 (m, 2H), 0.21-0.18 (m, 1H).

### Example 44

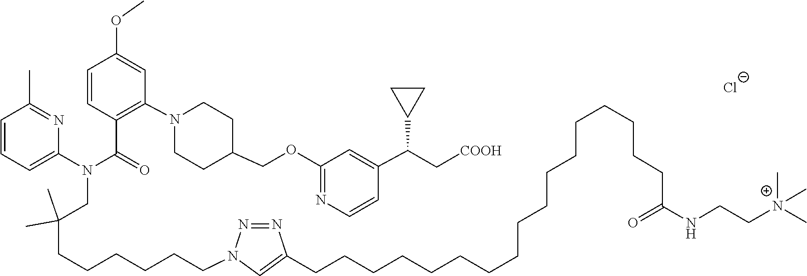 [SMILES: COc1ccc(C(=O)N(CC(C)(C)CCCCCCn2cc(CCCCCCCCCCCCCCCCCC(=O)NCC[N+](C)(C)C)nn2)c2cccc(C)n2)c(N2CCC(COc3cc([C@@H](CC(=O)O)C4CC4)ccn3)CC2)c1 ; [Cl-]]

MS (ESI) m/z 1118.5 [M]⁺. ¹H NMR (400 MHZ, DMSO-d₆): δ 8.14-8.12 (t, NH, 2H), 8.04-8.03 (d, 1H), 7.79 (s, 1H), 7.27-7.24 (br, 1H), 7.15-7.11 (d, 1H), 6.92-6.91 (d, 1H), 6.84-6.83 (d, 1H), 6.71 (s, 1H), 6.52-6.50 (d, 1H), 6.40 (br, 1H), 6.21 (s, 1H), 4.24-4.20 (t, 2H), 4.13-4.11 (d, 2H), 4.08 (br, 2H), 3.70 (s, 3H), 3.47-3.44 (m, 2H), 3.35-3.31 (m, 2H), 3.08 (s, 9H), 2.67-2.62 (d, 2H), 2.58-2.55 (t, 2H), 2.33 (s, 3H), 2.25-2.20 (m, 1H), 2.12-2.08 (t, 2H), 1.75-1.62 (m, 5H), 1.60-1.30 (m, 6H), 1.30-1.15 (m, 26H), 1.12-0.90 (m, 9H), 0.75 (s, 6H), 0.55-0.50 (m, 1H), 0.34-0.29 (m, 2H), 0.21-0.18 (m, 1H).

### Example 45

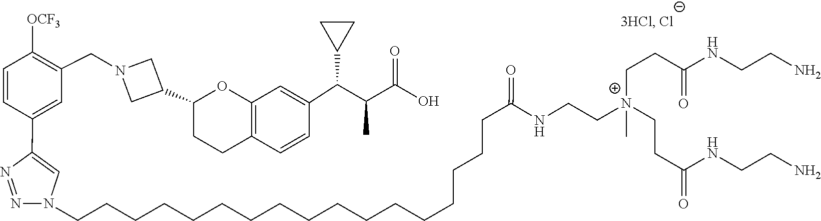 [SMILES: C[C@H](C(=O)O)[C@H](c1ccc2c(c1)O[C@@H](C1CN(Cc3cc(-c4cn(CCCCCCCCCCCCCCCCCC(=O)NCC[N+](C)(CCC(=O)NCCN)CCC(=O)NCCN)nn4)ccc3OC(F)(F)F)C1)CC2)C1CC1]

MS (ESI) m/z 1125.0 [M]⁺. ¹H NMR (400 MHZ, DMSO-d₆): δ 8.75 (s, 1H), 8.42 (t, NH, 2H), 8.20 (s, 1H), 8.18 (t, NH, 1H), 8.00-7.96 (d, 1H), 7.90 (br, 6H), 7.59-7.57 (d, 1H), 7.01-6.99 (d, 1H), 6.70-6.68 (d, 1H), 6.63 (s, 1H), 4.63 (br, 1H), 4.43-4.40 (t, 2H), 4.29-4.27 (m, 2H), 4.27-4.14 (m, 3H), 3.70-3.50 (m, 6H), 3.34-3.29 (m, 6H), 3.14-3.11 (m, 1H), 3.03 (s, 3H), 2.89-2.82 (m, 4H), 2.80-2.76 (m, 1H), 2.76-65 (m, 6H), 2.10-2.07 (t, 2H), 1.87-1.84 (m, 4H), 1.56-1.47 (m, 4H), 1.30-1.17 (m, 26H), 1.07-1.00 (m, 1H), 0.82-0.79 (d, 3H), 0.55-0.50 (m, 1H), 0.26-0.19 (m, 2H), −0.87˜−0.82 (m, 1H).

### Example 46

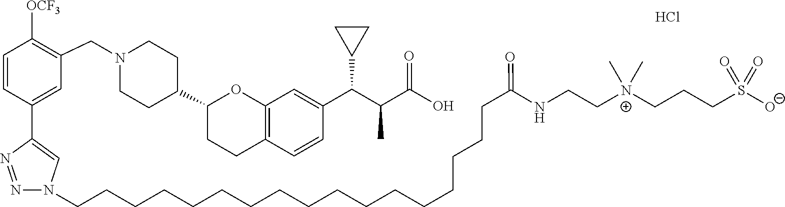 [SMILES: C[C@H](C(=O)O)[C@H](c1ccc2c(c1)O[C@@H](C1CCN(Cc3cc(-c4cn(CCCCCCCCCCCCCCCCCC(=O)NCC[N+](C)(C)CCCS(=O)(=O)[O-])nn4)ccc3OC(F)(F)F)CC1)CC2)C1CC1 ; Cl]

MS (ESI) m/z 1059.7 [M+H]⁺. ¹H NMR (400 MHZ, DMSO-d₆): δ 8.69 (s, 1H), 8.49-8.48 (s, 1H), 8.18-8.16 (m, 1H), 8.05-8.02 (d, 1H), 7.61-7.59 (d, 1H), 6.99-6.97 (d, 1H), 6.67-6.65 (d, 1H), 6.53 (s, 1H), 4.46-4.42 (m, 4H), 3.83-3.81 (m, 1H), 3.5-3.26 (m, 8H), 3.18-3.11 (m, 2H), 3.06 (s, 6H), 2.86-2.63 (m, 3H), 2.55-2.48 (m, 2H), 2.11-1.63 (m, 14H), 1.54-1.48 (m, 2H), 1.35-1.16 (m, 26H), 1.15-1.10 (m, 1H), 0.88-0.85 (d, 3H), 0.54-0.50 (m, 1H), 0.27-0.24 (m, 2H), −0.07 ˜−0.13 (m, 1H).

### Example 47

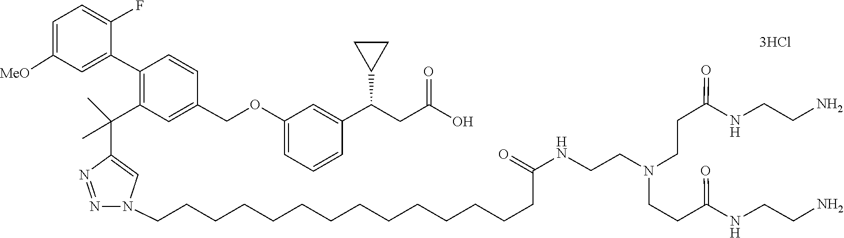 [SMILES: COc1ccc(F)c(-c2ccc(COc3cccc([C@@H](CC(=O)O)C4CC4)c3)cc2C(C)(C)c2cn(CCCCCCCCCCCCCCC(=O)NCCN(CCC(=O)NCCN)CCC(=O)NCCN)nn2)c1]

MS (ESI) m/z 1040.8 [M+H]⁺. ¹H NMR (400 MHZ, DMSO-d₆): δ 10.2 (br, 1H), 8.48-8.45 (t, NH, 2H), 8.20-8.18 (t, NH, 1H), 8.01 (br, 6H), 7.69 (s, 1H), 7.36-7.34 (d, 1H), 7.31 (s, 1H), 7.25-7.21 (t, 1H), 7.00-6.85 (m, 5H), 6.80-6.78 (m, 1H), 5.94-5.93 (m, 1H), 5.14 (s, 2H), 4.11-4.05 (m, 2H), 3.60 (s, 3H), 3.5 (m, 2H), 3.38-3.36 (m, 4H), 3.34-3.31 (m, 4H), 3.16 (br, 2H), 2.88-2.86 (m, 4H), 2.70-2.64 (m, 6H), 2.28-2.2.25 (m, 1H), 2.11-2.07 (t, 2H), 1.72-1.70 (m, 2H), 1.59 (s, 3H), 1.46 (s, 3H), 1.47-1.44 (m, 2H), 1.36-1.14 (m, 20H), 1.06-1.01 (m, 1H), 0.52-0.49 (m, 1H), 0.35-0.19 (m, 2H), 0.13-0.10 (m, 1H).

### Example 48

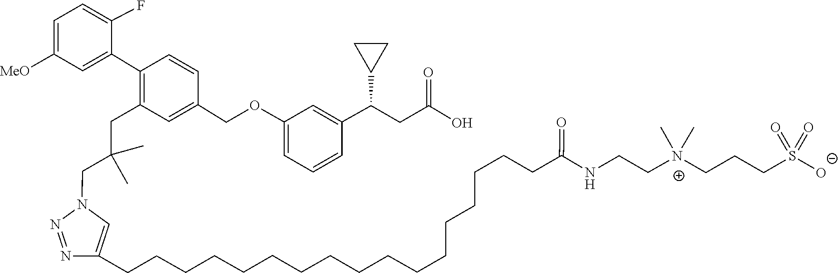 [SMILES: COc1ccc(F)c(-c2ccc(COc3cccc([C@@H](CC(=O)O)C4CC4)c3)cc2CC(C)(C)Cn2cc(CCCCCCCCCCCCCCCCCC(=O)NCC[N+](C)(C)CCCS(=O)(=O)[O-])nn2)c1]

MS (ESI) m/z 1032.8 [M+H]⁺. ¹H NMR (400 MHZ, DMSO-d₆): δ 8.12-8.10 (t, NH, 1H), 7.62 (s, 1H), 7.43 (s, 1H), 7.41-7.39 (d, 1H), 7.23-7.12 (m, 3H), 6.95-6.91 (m, 2H), 6.86-6.81 (m, 2H), 6.79-6.76 (m, 1H), 5.13 (s, 2H), 3.97 (s, 2H), 3.73 (s, 3H), 3.45-3.28 (m, 6H), 3.04 (s, 6H), 2.73-2.67 (m, 1H), 2.65-2.62 (m, 2H), 2.60-2.56 (t, 2H), 2.52-2.46 (m, 2H), 2.26-2.23 (m, 1H), 2.10-2.07 (t, 2H), 2.02-1.96 (m, 2H), 1.55-1.46 (m, 4H), 1.30-1.20 (m, 26H), 1.00-0.90 (m, 1H), 0.55 (s, 3H), 0.52 (s, 3H), 0.52-0.46 (m, 1H), 0.29-0.18 (m, 2H), 0.11-0.07 (m, 1H).

### Example 49

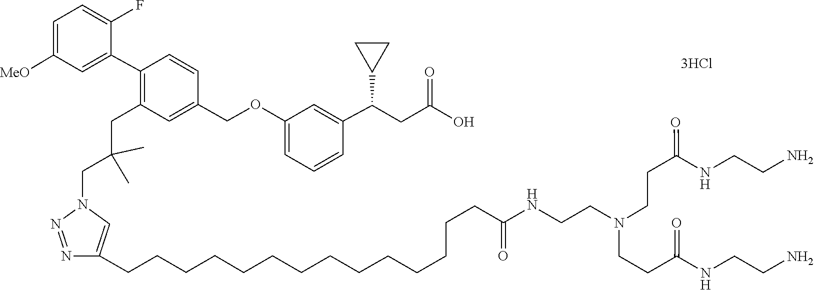 [SMILES: COc1ccc(F)c(-c2ccc(COc3cccc([C@@H](CC(=O)O)C4CC4)c3)cc2CC(C)(C)Cn2cc(CCCCCCCCCCCCCCC(=O)NCCN(CCC(=O)NCCN)CCC(=O)NCCN)nn2)c1]

MS (ESI) m/z 1068.9 [M+H]⁺. ¹H NMR (400 MHZ, DMSO-d₆): δ 8.42 (t, NH, 2H), 8.15 (t, NH, 1H), 7.91 (br, 6H), 7.60 (s, 1H), 7.43 (s, 1H), 7.42-7.39 (d, 1H), 7.24-7.12 (m, 3H), 6.95-6.91 (m, 2H), 6.86-6.82 (m, 2H), 6.79-6.77 (m, 1H), 5.14 (s, 2H), 3.98 (s, 2H), 3.74 (s, 3H), 3.50-3.37 (m, 6H), 3.32-3.29 (m, 4H), 3.17-3.13 (m, 2H), 2.89-2.86 (m, 4H), 2.73-2.68 (m, 1H), 2.65-2.60 (m, 6H), 2.58-2.55 (t, 2H), 2.55-2.49 (m, 1H), 2.28-2.23 (m, 1H), 2.10-2.06 (t, 2H), 1.54-1.47 (m, 4H), 1.30-1.20 (m, 20H), 1.01-0.97 (m, 1H), 0.55 (s, 3H), 0.52 (s, 3H), 0.51-47 (m, 1H), 0.30-0.19 (m, 2H), 0.11-0.07 (m, 1H).

### Example 50

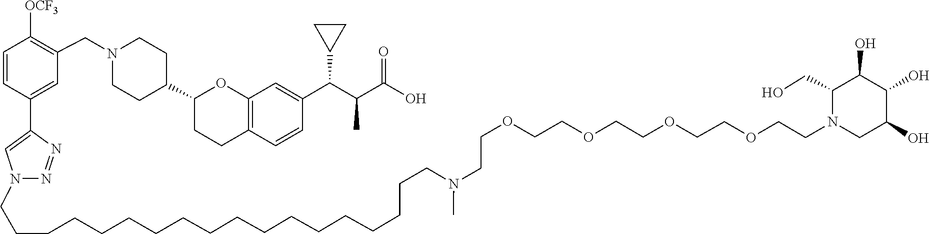 [SMILES: C[C@H](C(=O)O)[C@H](c1ccc2c(c1)O[C@@H](C1CCN(Cc3cc(-c4cn(CCCCCCCCCCCCCCCCCCN(C)CCOCCOCCOCCOCCN5C[C@H](O)[C@@H](O)[C@H](O)[C@H]5CO)nn4)ccc3OC(F)(F)F)CC1)CC2)C1CC1]

MS (ESI) m/z 1233.0 [M+H]⁺.

### Example 51

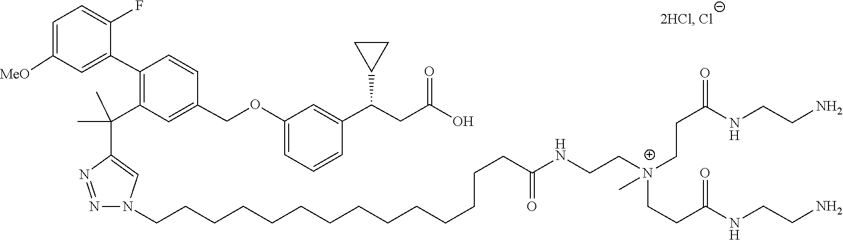 [SMILES: COc1ccc(F)c(-c2ccc(COc3cccc([C@@H](CC(=O)O)C4CC4)c3)cc2C(C)(C)c2cn(CCCCCCCCCCCCCCC(=O)NCC[N+](C)(CCC(=O)NCCN)CCC(=O)NCCN)nn2)c1]

MS (ESI) m/z 1054.9 [M]⁺. ¹H NMR (400 MHZ, DMSO-d₆): δ 8.45 (t, NH, 2H), 8.12 (t, NH, 1H), 7.92 (br, 6H), 7.68 (s, 1H), 7.35-7.33 (d, 1H), 7.30 (s, 1H), 7.24-7.20 (t, 1H), 7.01-6.85 (m, 5H), 6.81-6.77 (m, 1H), 5.95-5.93 (m, 1H), 5.14 (s, 2H), 4.10-4.03 (m, 2H), 3.60 (s, 3H), 3.60-3.54 (m, 4H), 3.50-3.44 (m, 2H), 3.35-3.29 (m, 6H), 3.05 (s, 3H), 2.90-2.84 (m, 4H), 2.71-2.64 (m, 6H), 2.30-2.25 (m, 1H), 1.73-1.69 (m, 2H), 1.60 (s, 3H), 1.49-1.44 (m, 2H), 1.44 (s, 3H), 1.30-1.20 (m, 20H), 1.03-1.00 (m, 1H), 0.51-0.48 (m, 1H), 0.34-0.25 (m, 2H), 0.14-0.09 (m, 1H).

### Example 52

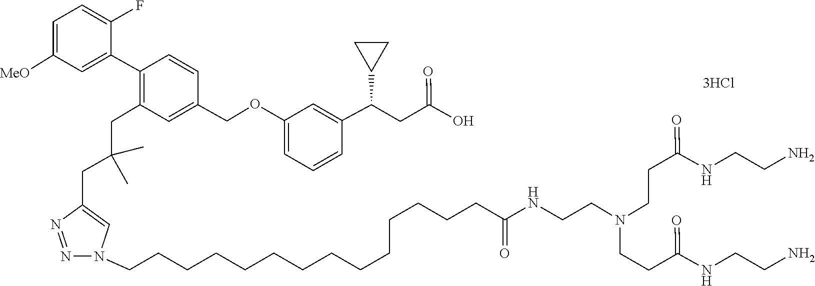 [SMILES: COc1ccc(F)c(-c2ccc(COc3cccc([C@@H](CC(=O)O)C4CC4)c3)cc2CC(C)(C)Cc2cn(CCCCCCCCCCCCCCC(=O)NCCN(CCC(=O)NCCN)CCC(=O)NCCN)nn2)c1]

MS (ESI) m/z 1069.9 [M+H]⁺. ¹H NMR (400 MHZ, DMSO-d₆): δ 10.22 (br, 1H), 8.50 (t, NH, 2H), 8.22 (t, NH, 1H), 8.07 (br, 6H), 7.71 (s, 1H), 7.45 (s, 1H), 7.40-7.37 (d, 1H), 7.22-7.14 (m, 3H), 6.96-6.92 (m, 2H), 6.87-82 (m, 2H), 6.80-6.77 (m, 1H), 5.13 (s, 2H), 4.26-4.23 (t, 2H), 3.74 (s, 3H), 3.44-3.40 (m, 2H), 3.40-3.36 (m, 4H), 3.36-3.32 (m, 4H), 3.18-3.15 (m, 2H), 2.90-2.86 (m, 4H), 2.71-2.62 (m, 6H), 2.26-2.23 (m, 1H), 2.11-2.07 (t, 2H), 1.77-1.73 (m, 2H), 1.50-1.46 (m, 2H), 1.30-1.20 (m, 20H), 1.01-0.98 (m, 1H), 0.55 (s, 3H), 0.53 (s, 3H), 0.51-0.46 (m, 1H), 0.31-0.17 (m, 2H), 0.11-0.07 (m, 1H).

### Example 53

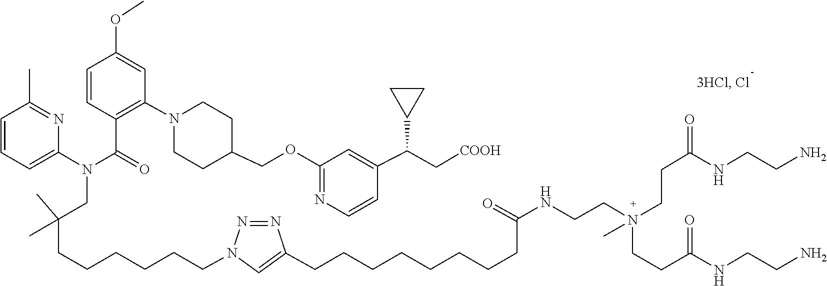

MS (ESI) m/z 1193.0 [M]⁺. ¹H NMR (400 MHZ, DMSO-d₆): δ 8.61 (t, NH, 2H), 8.25 (t, NH, 1H), 8.10 (br, 6H), 8.09-8.06 (d, 1H), 7.83 (s, 1H), 7.30 (br, 1H), 7.11-7.09 (d, 1H), 6.98-6.95 (br, 1H), 6.86-6.84 (br, 1H), 6.78-6.74 (m, 1H), 6.53-6.51 (d, 1H), 6.51-6.40 (br, 1H), 6.26-6.22 (br, 1H), 4.24-4.17 (t, 2H), 4.14-4.12 (d, 2H), 4.12-4.08 (br, 2H), 3.70 (s, 3H), 3.64-3.60 (m, 4H), 3.50-3.45 (m, 2H), 3.37-3.31 (m, 6H), 3.08 (s, 3H), 2.90-2.85 (m, 4H), 2.75-2.68 (m, 6H), 2.60-2.55 (t, 2H), 2.34 (s, 3H), 2.27-2.24 (m, 1H), 2.11-2.08 (t, 2H), 1.77-1.73 (m, 1H), 1.69-1.64 (m, 4H), 1.60-1.31 (m, 6H), 1.31-1.15 (m, 8H), 1.12-0.90 (m, 9H), 0.74 (s, 6H), 0.53-0.49 (m, 1H), 0.34-0.29 (m, 2H), 0.18-0.14 (m, 1H).

### Example 54

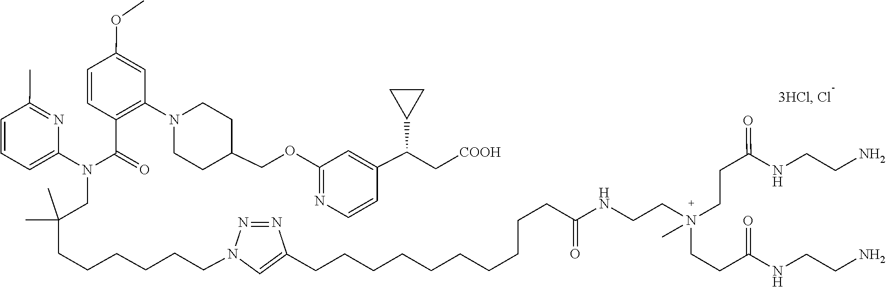

MS (ESI) m/z 611.2 [(M+H)/2]⁺. ¹H NMR (400 MHZ, DMSO-d₆): δ 8.57 (t, NH, 2H), 8.31 (t, NH, 1H), 8.14 (br, 6H), 8.10-8.07 (d, 1H), 7.83 (s, 1H), 7.33-7.30 (t, 1H), 7.12-7.09 (d, 1H), 6.99 (br, 1H), 6.88-6.85 (d, 1H), 6.81-6.79 (s, 1H), 6.55-6.53 (d, 1H), 6.52-6.48 (br, 1H), 6.26 (s, 1H), 4.26-4.25 (m, 2H), 4.16-4.14 (d, 2H), 4.08 (br, 2H), 3.70 (s, 3H), 3.63-3.59 (m, 4H), 3.48-3.45 (m, 2H), 3.35-3.31 (m, 6H), 3.07 (s, 3H), 2.90-2.85 (m, 4H), 2.74-2.69 (m, 6H), 2.60-2.55 (t, 2H), 2.34 (s, 3H), 2.30-2.24 (m, 1H), 2.11-2.08 (t, 2H), 1.78-1.73 (m, 1H), 1.68-1.63 (m, 4H), 1.56-1.31 (m, 6H), 1.29-1.18 (m, 12H), 1.11-0.90 (m, 9H), 0.72 (s, 6H), 0.54-0.49 (m, 1H), 0.35-0.26 (m, 2H), 0.18-0.14 (m, 1H).

### Example 55

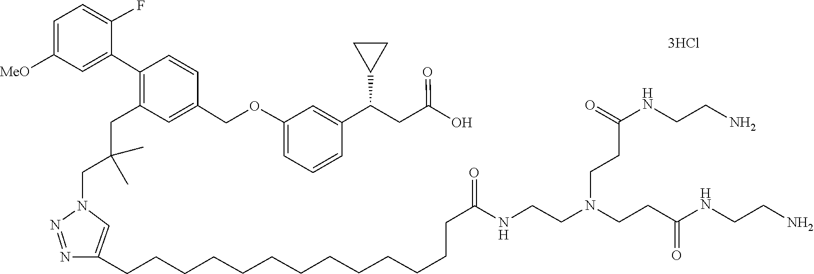 [SMILES: COc1ccc(F)c(-c2ccc(COc3cccc([C@@H](CC(=O)O)C4CC4)c3)cc2CC(C)(C)Cn2cc(CCCCCCCCCCCCCC(=O)NCCN(CCC(=O)NCCN)CCC(=O)NCCN)nn2)c1]

MS (ESI) m/z 1054.9 [M+H]⁺. ¹H NMR (400 MHZ, DMSO-d₆): δ 10.15 (br, 1H), 8.48 (t, NH, 2H), 8.20 (t, NH, 1H), 8.03 (br, NH₃, 6H), 7.63 (s, 1H), 7.44 (s, 1H), 7.42-7.40 (d, 1H), 7.23-7.12 (m, 3H), 6.95-6.91 (m, 2H), 6.86-6.82 (m, 2H), 6.79-6.77 (m, 1H), 5.14 (s, 2H), 3.98 (s, 2H), 3.74 (s, 3H), 3.44-3.38 (m, 2H), 3.37-3.34 (m, 4H), 3.34-3.30 (m, 4H), 3.18-3.16 (m, 2H), 2.91-2.86 (m, 4H), 2.71-2.62 (m, 7H), 2.57-2.53 (m, 3H), 2.26-2.23 (m, 1H), 2.11-2.07 (t, 2H), 1.54-1.46 (m, 4H), 1.30-1.22 (m, 18H), 1.01-0.97 (m, 1H), 0.56 (s, 3H), 0.52 (s, 3H), 0.52-0.49 (m, 1H), 0.32-0.17 (m, 2H), 0.12-0.07 (m, 1H).

### Example 56

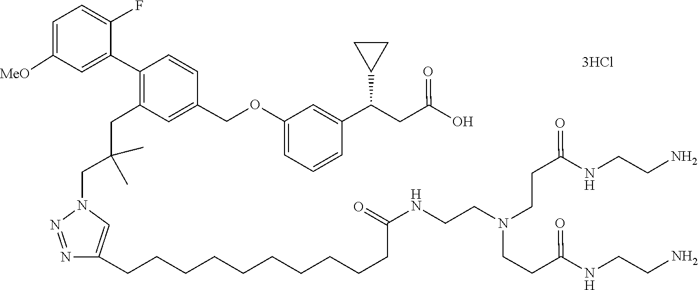 [SMILES: COc1ccc(F)c(-c2ccc(COc3cccc([C@@H](CC(=O)O)C4CC4)c3)cc2CC(C)(C)Cn2cc(CCCCCCCCCCC(=O)NCCN(CCC(=O)NCCN)CCC(=O)NCCN)nn2)c1]

MS (ESI) m/z 1013.8 [M+H]⁺. ¹H NMR (400 MHZ, DMSO-d₆): δ 10.07 (br, 1H), 8.43 (t, NH, 2H), 8.19 (t, NH, 1H), 8.00 (br, NH₃, 6H), 7.63 (s, 1H), 7.44 (s, 1H), 7.42-7.40 (d, 1H), 7.24-7.12 (m, 3H), 6.95-6.91 (m, 2H), 6.87-6.82 (m, 2H), 6.80-6.77 (m, 1H), 5.14 (s, 2H), 3.98 (s, 2H), 3.74 (s, 3H), 3.45-3.42 (m, 2H), 3.42-3.37 (m, 4H), 3.36-3.30 (m, 4H), 3.19-3.15 (m, 2H), 2.91-2.86 (m, 4H), 2.69-2.62 (m, 7H), 2.57-2.51 (m, 3H), 2.26-2.23 (m, 1H), 2.11-2.07 (t, 2H), 1.54-1.46 (m, 4H), 1.30-1.22 (m, 18H), 1.00-0.96 (m, 1H), 0.56 (s, 3H), 0.52 (s, 3H), 0.52-0.49 (m, 1H), 0.32-0.17 (m, 2H), 0.12-0.07 (m, 1H).

### Example 57

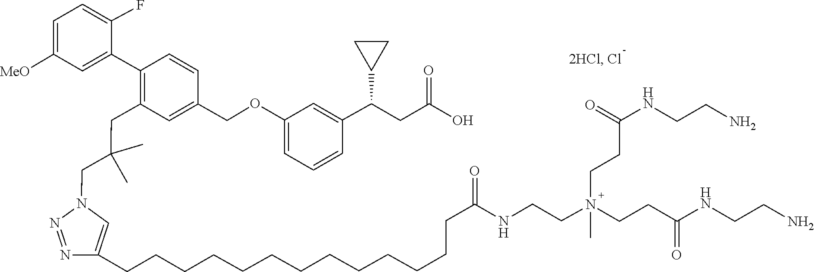

MS (ESI) m/z 534.8 [(M+H)/2]⁺. ¹H NMR (400 MHZ, DMSO-d₆): δ 8.57 (t, NH, 2H), 8.27 (t, NH, 1H), 8.12 (br, NH₃, 6H), 7.62 (s, 1H), 7.44 (s, 1H), 7.42-7.39 (d, 1H), 7.23-7.12 (m, 3H), 6.94-6.91 (m, 2H), 6.86-6.81 (m, 2H), 6.79-6.76 (m, 1H), 5.13 (s, 2H), 3.97 (s, 2H), 3.76 (s, 3H), 3.63-3.59 (m, 4H), 3.50-3.45 (m, 2H), 3.36-3.31 (m, 6H), 3.06 (s, 3H), 2.90-2.85 (m, 4H), 2.74-2.70 (m, 5H), 2.67-2.63 (m, 2H), 2.61-2.54 (m, 3H), 2.28-2.21 (m, 1H), 2.10-2.07 (t, 2H), 1.55-1.46 (m, 4H), 1.26-1.20 (m, 18H), 1.00-0.97 (m, 1H), 0.56 (s, 3H), 0.52 (s, 3H), 0.52-0.49 (m, 1H), 0.32-0.17 (m, 2H), 0.12-0.07 (m, 1H).

### Example 58

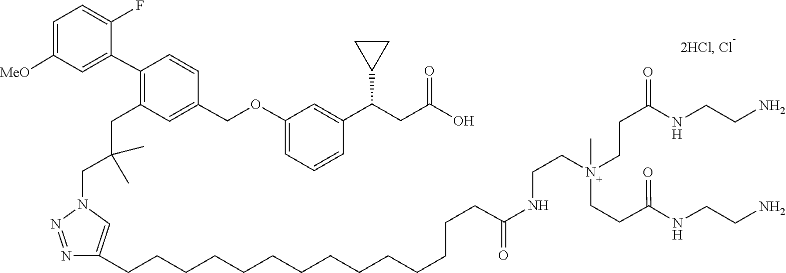

MS (ESI) m/z 1082.9 [(M+H)/2]⁺. ¹H NMR (400 MHZ, DMSO-d₆): δ 8.43 (t, NH, 2H), 8.17 (t, NH, 1H), 7.95 (br, NH₃, 6H), 7.63 (s, 1H), 7.44 (s, 1H), 7.42-7.40 (d, 1H), 7.23-7.12 (m, 3H), 6.94-6.91 (m, 2H), 6.86-6.82 (m, 2H), 6.79-6.77 (m, 1H), 5.14 (s, 2H), 3.98 (s, 2H), 3.73 (s, 3H), 3.62-3.59 (m, 4H), 3.46-3.45 (m, 2H), 3.34-3.29 (m, 6H), 3.04 (s, 3H), 2.90-2.85 (m, 4H), 2.73-2.60 (m, 7H), 2.57-2.51 (m, 3H), 2.28-2.22 (m, 1H), 2.11-2.07 (t, 2H), 1.56-1.46 (m, 4H), 1.31-1.22 (m, 20H), 1.00-0.97 (m, 1H), 0.56 (s, 3H), 0.52 (s, 3H), 0.52-0.49 (m, 1H), 0.32-0.17 (m, 2H), 0.12-0.07 (m, 1H).

### Example 59

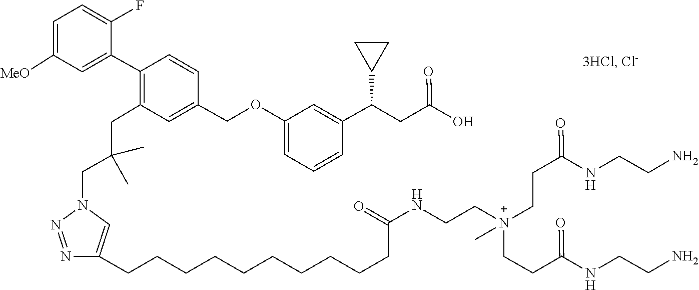 [SMILES: COc1ccc(F)c(-c2ccc(COc3cccc([C@@H](CC(=O)O)C4CC4)c3)cc2CC(C)(C)Cn2cc(CCCCCCCCCCC(=O)NCC[N+](C)(CCC(=O)NCCN)CCC(=O)NCCN)nn2)c1]

MS (ESI) m/z 1026.8 [(M+H)/2]⁺. ¹H NMR (400 MHZ, DMSO-d₆): δ 8.50 (t, NH, 2H), 8.22 (t, NH, 1H), 8.02 (br, NH₃, 6H), 7.63 (s, 1H), 7.43 (s, 1H), 7.42-7.40 (d, 1H), 7.23-7.12 (m, 3H), 6.95-6.91 (m, 2H), 6.86-6.82 (m, 2H), 6.79-6.77 (m, 1H), 5.14 (s, 2H), 3.98 (s, 2H), 3.74 (s, 3H), 3.62-3.58 (m, 4H), 3.46-3.45 (m, 2H), 3.35-3.30 (m, 6H), 3.06 (s, 3H), 2.90-2.86 (m, 4H), 2.73-2.67 (m, 5H), 2.65-2.60 (m, 2H), 2.57-2.53 (m, 3H), 2.28-2.22 (m, 1H), 2.11-2.07 (t, 2H), 1.55-1.46 (m, 4H), 1.28-1.24 (m, 12H), 1.00-0.97 (m, 1H), 0.56 (s, 3H), 0.52 (s, 3H), 0.52-0.49 (m, 1H), 0.32-0.17 (m, 2H), 0.12-0.07 (m, 1H).

### Example 60

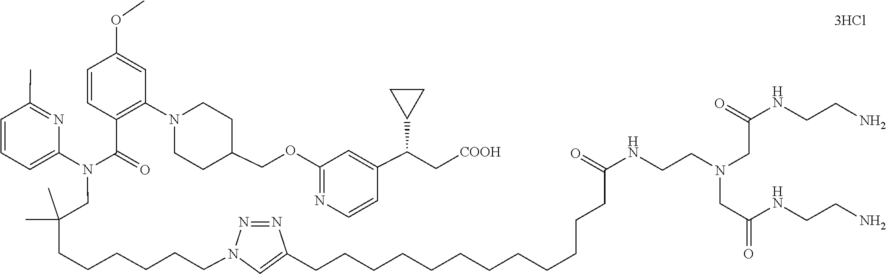 [SMILES: COc1ccc(C(=O)N(CC(C)(C)CCCCCCn2cc(CCCCCCCCCCCCC(=O)NCCN(CC(=O)NCCN)CC(=O)NCCN)nn2)c2cccc(C)n2)c(N2CCC(COc3cc([C@@H](CC(=O)O)C4CC4)ccn3)CC2)c1]

MS (ESI) m/z 1208.1 [M+H]⁺. ¹H NMR (400 MHZ, DMSO-d₆): δ 8.98 (t, NH, 2H), 8.22 (t, NH, 1H), 8.11 (br, NH₃, 6H), 8.11-8.10 (d, 1H), 7.87 (s, 1H), 7.40-7.29 (br, 1H), 7.18-7.16 (d, 1H), 7.04-7.03 (d, 1H), 6.91-6.86 (m, 1H), 6.86 (s, 1H), 6.56-6.53 (m, 2H), 6.31 (s, 1H), 4.26-4.23 (t, 2H), 4.21-4.19 (d, 2H), 4.17-4.13 (br, 2H), 4.02 (br, 2H), 3.69 (s, 3H), 3.41-3.38 (m, 6H), 3.32 (br, 2H), 2.93-2.89 (m, 4H), 2.74-2.72 (d, 2H), 2.61-2.57 (t, 2H), 2.35 (s, 3H), 2.30-2.25 (m, 1H), 2.10-2.08 (t, 2H), 1.81-1.78 (br, 1H), 1.72-1.66 (m, 4H), 1.57-1.54 (m, 2H), 1.49-1.40 (m, 4H), 1.31-1.17 (m, 16H), 1.12-0.97 (m, 5H), 0,.75 (s, 6H), 0.55-0.50 (m, 1H), 0.37-0.26 (m, 2H), 0.20-0.14 (m, 1H).

### Example 61

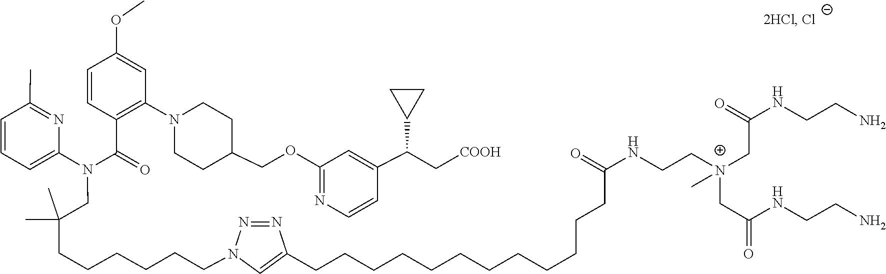 [SMILES: COc1ccc(C(=O)N(CC(C)(C)CCCCCCn2cc(CCCCCCCCCCCCC(=O)NCC[N+](C)(CC(=O)NCCN)CC(=O)NCCN)nn2)c2cccc(C)n2)c(N2CCC(COc3cc([C@@H](CC(=O)O)C4CC4)ccn3)CC2)c1]

MS (ESI) m/z 1022.0 [M+H]⁺. ¹H NMR (400 MHZ, DMSO-d₆): δ 9.10 (t, NH, 2H), 8.38 (t, NH, 1H), 8.23 (br, NH₃, 6H), 8.12-8.10 (d, 1H), 7.88 (s, 1H), 7.36-7.34 (br, 1H), 7.13-7.11 (d, 1H), 7.05-7.04 (d, 1H), 6.92-6.90 (m, 1H), 6.90 (s, 1H), 6.56-6.54 (m, 2H), 6.33 (s, 1H), 4.44 (s, 4H), 4.27-4.23 (t, 2H), 4.18-4.17 (d, 2H), 4.13 (br, 2H), 3.75 (br, 2H), 3.68 (s, 3H), 3.56-3.53 (m, 2H), 3.42 (s, 3H), 3.40-3.38 (m, 4H), 2.94-2.89 (m, 4H), 2.74-2.72 (d, 2H), 2.61-2.57 (t, 2H), 2.35 (s, 3H), 2.33-2.26 (m, 1H), 2.12-2.01 (m, 2H), 1.82 (br, 1H), 1.72-1.67 (m, 4H), 1.58-1.55 (m, 2H), 1.47-1.39 (m, 4H), 1.31-1.17 (m, 16H), 1.12-0.97 (m, 5H), 0.75 (s, 6H), 0.55-0.50 (m, 1H), 0.37-0.26 (m, 2H), 0.20-0.14 (m, 1H).

### Example 62

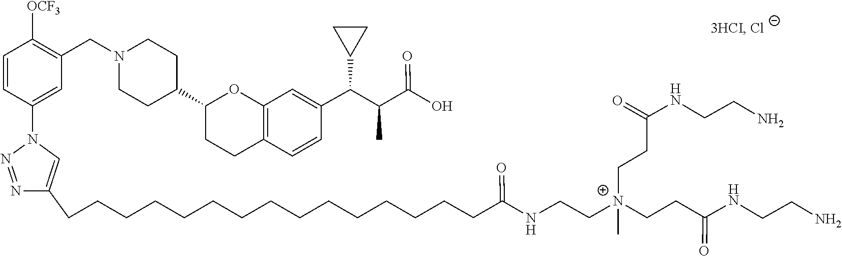 [SMILES: C[C@H](C(=O)O)[C@H](c1ccc2c(c1)O[C@@H](C1CCN(Cc3cc(-n4cc(CCCCCCCCCCCCCCCC(=O)NCC[N+](C)(CCC(=O)NCCN)CCC(=O)NCCN)nn4)ccc3OC(F)(F)F)CC1)CC2)C1CC1]

MS (ESI) m/z 1095.8 [(M+H)/2]⁺. ¹H NMR (400 MHZ, DMSO-d₆): δ 8.69 (m, 2H), 8.57 (t, 2H), 8.28 (t, NH, 1H), 8.13 (br, NH₃, 6H), 7.73-7.71 (d, 1H), 6.97-6.95 (d, 1H), 6.66-6.64 (d, 1H), 6.52 (s, 1H), 4.49 (m, 2H), 3.81-3.79 (m, 1H), 3.64-3.60 (m, 4H), 3.47 (br, 2H), 3.40-3.30 (br, 8H), 3.20-3.10 (br, 2H), 3.06 (s, 3H), 2.89-2.81 (m, 4H), 2.72-2.63 (m, 9H), 2.15-1.82 (m, 8H), 1.70-1.59 (m, 4H), 1.50-1.40 (m, 2H), 1.35-1.20 (m, 18H), 1.10-1.00 (m, 1H), 0.80 (d, 3H), 0.52-0.49 (m, 1H), 0.25-0.20 (m, 2H), −0.10 ˜−0.13 (m, 1H).

### Example 63

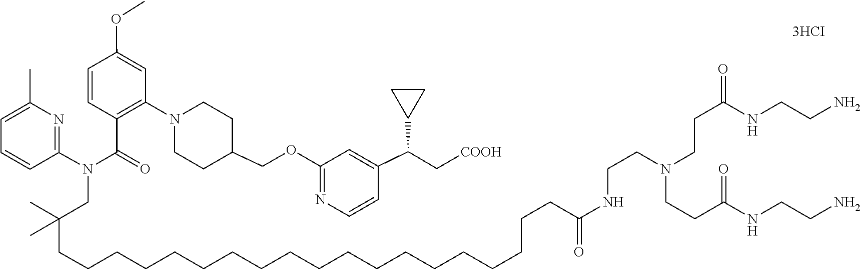 [SMILES: COc1ccc(C(=O)N(CC(C)(C)CCCCCCCCCCCCCCCCCCCC(=O)NCCN(CCC(=O)NCCN)CCC(=O)NCCN)c2cccc(C)n2)c(N2CCC(COc3cc([C@@H](CC(=O)O)C4CC4)ccn3)CC2)c1]

MS (ESI) m/z 1183.0 [M+H]⁺. ¹H NMR (400 MHZ, DMSO-d₆): δ 8.36 (t, NH, 2H), 8.12 (t, NH, 1H), 8.04-8.03 (d, 1H), 7.81 (br, NH₃, 6H), 7.29-7.26 (m, 1H), 7.11-7.08 (d, 1H), 6.92-6.91 (d, 1H), 6.86-6.84 (d, 1H), 6.71 (s, 1H), 6.52-6.50 (d, 1H), 6.44 (br, 1H), 6.28 (br, 1H), 4.12 (m, 4H), 3.69 (s, 3H), 3.41-3.37 (m, 6H), 3.33-3.28 (m, 4H), 3.20-3.16 (m, 2H), 2.89-2.85 (m, 4H), 2.62-2.60 (d, 2H), 2.59-2.56 (m, 4H), 2.36 (s, 3H), 2.27-2.21 (m, 1H), 2.10-2.06 (t, 2H), 1.75 (br, 1H), 1.68-1.66 (m, 2H), 1.49-1.46 (m, 4H), 1.30-1.12 (m, 30H), 1.02-1.97 (m, 5H), 0.75 (s, 6H), 0.54-0.49 (m, 1H), 0.35-0.25 (m, 2H), 0.18-0.15 (m, 1H).

### Example 64

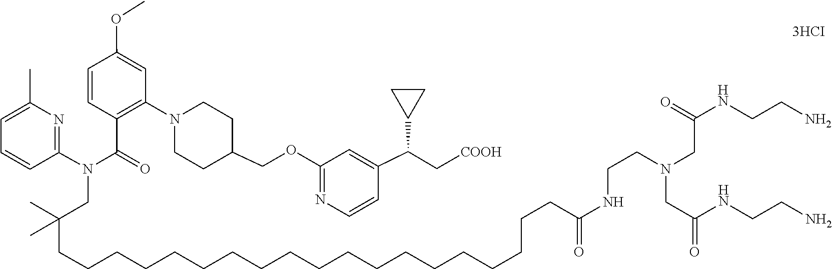 [SMILES: COc1ccc(C(=O)N(CC(C)(C)CCCCCCCCCCCCCCCCCCCC(=O)NCCN(CC(=O)NCCN)CC(=O)NCCN)c2cccc(C)n2)c(N2CCC(COc3cc([C@@H](CC(=O)O)C4CC4)ccn3)CC2)c1]

MS (ESI) m/z 1155.0 [M+H]⁺. ¹H NMR (400 MHZ, DMSO-d₆): δ 8.39 (t, NH, 2H), 8.04-8.03 (d, 1H), 7.93 (br, NH, 1H), 7.82 (br, NH₃, 6H), 7.26-7.24 (br, 1H), 7.10-7.08 (d, 1H), 6.92-6.91 (d, 1H), 6.86-6.84 (d, 1H), 6.70 (s, 1H), 6.52-6.50 (d, 1H), 6.44-6.43 (br, 1H), 6.22 (s, 1H), 4.13-4.11 (m, 4H), 3.68 (s, 3H), 3.53-3.50 (s, 4H), 3.36-3.31 (m, 6H), 3.26-3.20 (m, 2H), 2.90-2.88 (m, 4H), 2.69-2.67 (d, 2H), 2.36 (s, 3H), 2.26-2.22 (m, 1H), 2.08-2.04 (t, 2H), 1.76-1.74 (m, 1H), 1.68-1.65 (m, 2H), 1.46-1.40 (m, 4H), 1.30-1.12 (m, 30H), 1.02-0.97 (m, 5H), 0.75 (s, 6H), 0.52-0.49 (m, 1H), 0.35-0.25 (m, 2H), 0.18-0.15 (m, 1H).

### Example 65

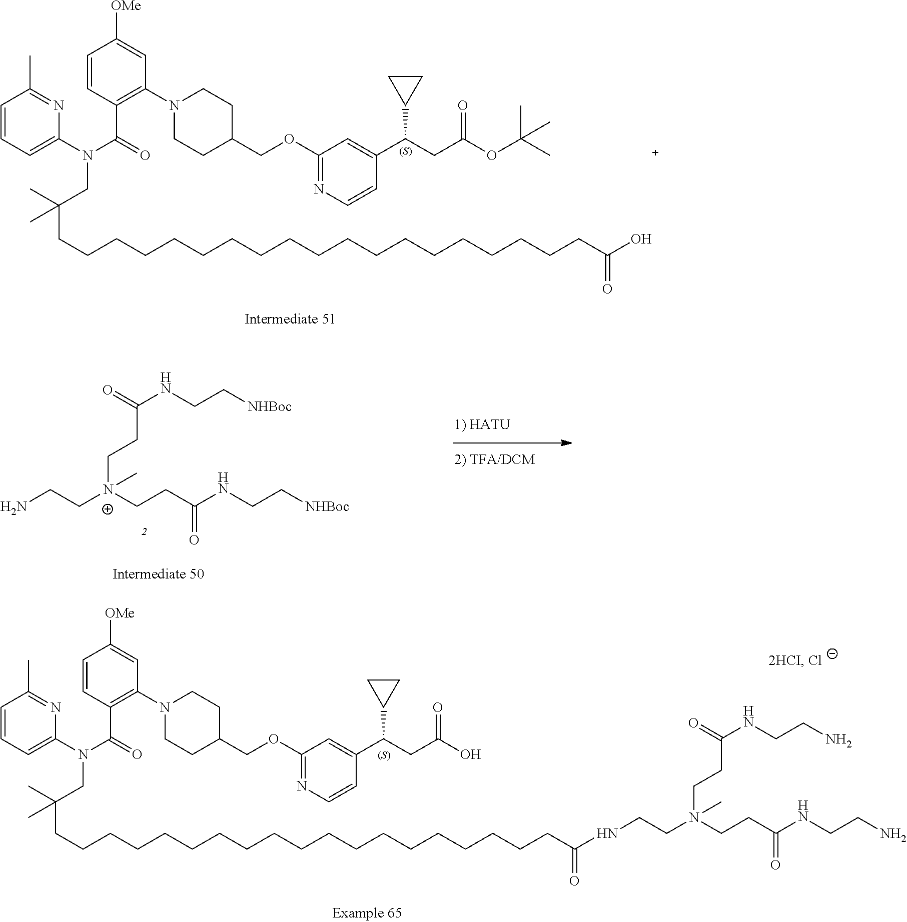

Step 1. To a mixture of Intermediate 51 (100 mg, 0.103 mmol) and Intermediate 50 (104 mg, 0.21 mmol) in THF (4 mL) was added HATU (79.8 mg, 0.21 mmol) and TEA (31 mg, 0.309 mmol). The resulting mixture was stirred at room temperature for 4 hrs. Solvent was concentrated and the residue was diluted with DCM/TFA (4 mL/2 mL). The resulting mixture was stirred at room temperature for 12 hrs. The reaction mixture was concentrated and the residue was purified by prep-HPLC (C8 column) to give(S)-3-((2-aminoethyl)amino)-N-(3-((2-aminoethyl)amino)-3-oxopropyl)-N-(2-(22-(2-(4-(((4-(2-carboxy-1-cyclopropylethyl) pyridin-2-yl)oxy)methyl) piperidin-1-yl)-4-methoxy-N-(6-methylpyridin-2-yl)benzamido)-21,21-dimethyldocosanamido)ethyl)-N-methyl-3-oxopropan-1-aminium chloride, Example 65, (10 mg, 0.8% yield) as a white solid. MS (ESI) m/z 1198.1 [M]⁺. ¹H NMR (400 MHZ, DMSO-d₆): δ 8.38 (t, NH, 2H), 8.15 (t, NH, 1H), 8.04-8.03 (d, 1H), 7.89 (br, NH₃, 6H), 7.27 (br, 1H), 7.10-7.08 (d, 1H), 6.92-6.91 (d, 1H), 6.86-6.84 (d, 1H), 6.71 (s, 1H), 6.52-6.51 (d, 1H), 6.50 (br, 1H), 6.22 (s, 1H), 4.14-4.12 (m, 4H), 3.68 (s, 3H), 3.59-3.56 (m, 4H), 3.46-3.44 (m, 2H), 3.33-3.24 (m, 6H), 3.03 (s, 3H), 2.90-2.85 (m, 4H), 2.69-2.67 (m, 6H), 2.36 (s, 3H), 2.27-2.20 (m, 1H), 2.10-2.06 (t, 2H), 1.75 (br, 1H), 1.68-1.65 (m, 2H), 1.49-1.46 (m, 4H), 1.35-1.14 (m, 30H), 1.02-0.97 (m, 5H), 0.75 (s, 6H), 0.53-0.49 (m, 1H), 0.35-0.25 (m, 2H), 0.18-0.15 (m, 1H).

Examples 63, 64, 67 were synthesized from acid and amine intermediates in the similar procedures as for Example 65.

### Example 66

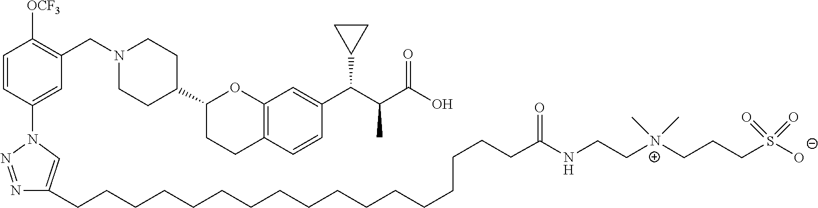 [SMILES: C[C@H](C(=O)O)[C@H](c1ccc2c(c1)O[C@@H](C1CCN(Cc3cc(-n4cc(CCCCCCCCCCCCCCCCCC(=O)NCC[N+](C)(C)CCCS(=O)(=O)[O-])nn4)ccc3OC(F)(F)F)CC1)CC2)C1CC1]

MS (ESI) m/z 1059.8 [(M+H)/2]⁺. ¹H NMR (400 MHZ, DMSO-d₆): δ 8.68 (t, NH, 2H), 8.59 (s, 1H), 8.16-8.10 (m, 2H), 7.74-7.72 (d, 1H), 6.97-6.95 (d, 1H), 6.66-6.64 (d, 1H), 6.52 (s, 1H), 4.47 (m, 2H), 3.50-3.39 (br, 7H), 3.37-3.30 (m, 2H), 3.20-3.10 (m, 2H), 3.05 (s, 3H), 2.79-2.60 (m, 5H), 2.50-2.46 (m, 4H), 2.11-2.07 (m, 3H), 2.02-1.96 (m, 3H), 1.94-1.60 (m, 8H), 1.47-1.37 (m, 2H), 1.31-1.15 (m, 26H), 1.08-1.10 (m, 1H), 0.81 (s, 3H), 0.52-0.49 (m, 1H), 0.25-0.20 (m, 2H), −0.10 ˜−0.13 (m, 1H).

### Example 67

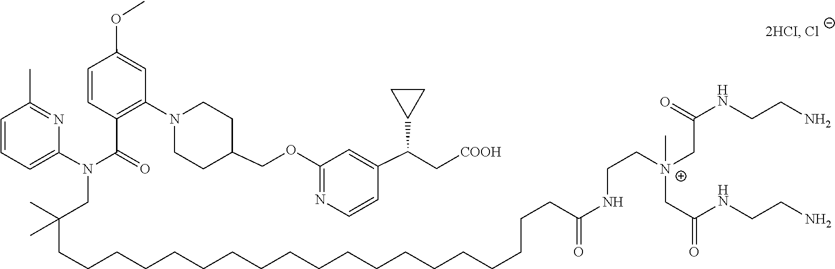 [SMILES: COc1ccc(C(=O)N(CC(C)(C)CCCCCCCCCCCCCCCCCCCC(=O)NCC[N+](C)(CC(=O)NCCN)CC(=O)NCCN)c2cccc(C)n2)c(N2CCC(COc3cc([C@@H](CC(=O)O)C4CC4)ccn3)CC2)c1]

MS (ESI) m/z 1026.8 [(M+H)/2]⁺. ¹H NMR (400 MHZ, DMSO-d₆): δ 8.92 (t, NH, 2H), 8.21 (t, NH, 1H), 8.04-8.03 (d, 1H), 7.93 (br, NH₃, 6H), 7.28-7.25 (br, 1H), 7.11-7.08 (d, 1H), 6.92-6.91 (d, 1H), 6.86-6.84 (d, 1H), 6.71 (s, 1H), 6.52-6.50 (d, 1H), 6.44-6.43 (br, 1H), 6.23 (s, 1H), 4.36 (s, 4H), 4.14-4.05 (m, 4H), 3.75-3.70 (m, 2H), 3.69 (s, 3H), 3.53-3.50 (m, 2H), 3.38 (s, 3H), 3.38-3.35 (m, 4H), 2.94-2.89 (m, 4H), 2.71-2.67 (m, 2H), 2.36 (s, 3H), 2.27-2.21 (m, 1H), 2.10-2.06 (t, 2H), 1.75 (br, 1H), 1.68-1.65 (m, 2H), 1.49-1.46 (m, 4H), 1.35-1.14 (m, 30H), 1.02-0.97 (m, 5H), 0.75 (s, 6H), 0.53-0.49 (m, 1H), 0.35-0.25 (m, 2H), 0.18-0.15 (m, 1H).

### Example 68

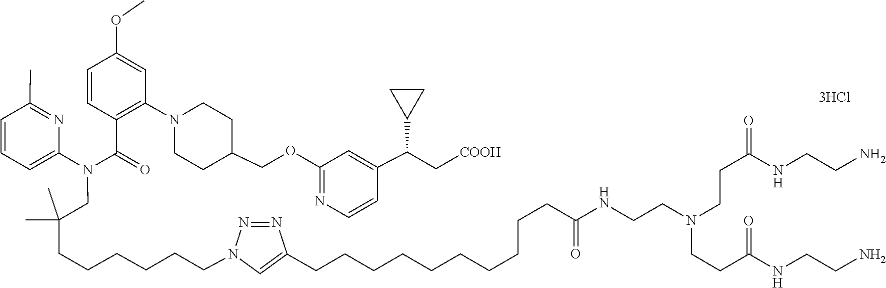 [SMILES: COc1ccc(C(=O)N(CC(C)(C)CCCCCCn2cc(CCCCCCCCCCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

MS (ESI) m/z 1234.4 [M+H]⁺. ¹H NMR (400 MHZ, DMSO-d₆): δ 8.47 (t, NH, 2H), 8.19 (t, NH, 1H), 8.06-8.05 (d, 1H), 7.99 (br, NH₃, 6H), 7.82 (s, 1H), 7.28 (br, 1H), 7.12-7.10 (d, 1H), 6.95-6.94 (d, 1H), 6.86-6.84 (d, 1H), 6.75 (s, 1H), 6.54-6.53 (d, 1H), 6.51 (br, 1H), 6.23 (s, 1H), 4.25-4.21 (t, 2H), 4.14-4.12 (d, 2H), 4.09 (br, 2H), 3.69 (s, 3H), 3.45-3.42 (t, 2H), 3.38-3.33 (m, 4H), 3.32-3.29 (m, 4H), 3.16 (br, 2H), 2.90-2.86 (m, 4H), 2.71-2.66 (m, 6H), 2.59-2.56 (t, 2H), 2.34 (s, 3H), 2.26-2.23 (m, 1H), 2.11-2.07 (t, 2H), 1.75-1.67 (m, 5H), 1.57-1.54 (m, 2H), 1.49-1.46 (m, 2H), 1.39 (br, 2H), 1.25 (br, 16H), 1.25-0.94 (m, 9H), 0.75 (s, 6H), 0.53-0.49 (m, 1H), 0.32-0.25 (m, 2H), 0.23-0.18 (m, 1H).

### Example 69

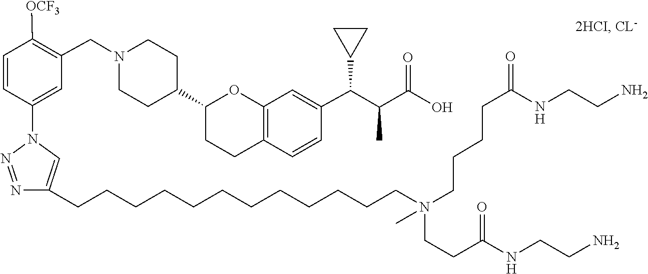

MS (ESI) m/z 1025.3 [M]⁺. ¹H NMR (400 MHZ, DMSO-d₆): δ 8.69 (s, 1H), 8.63 (s, 1H), 8.55 (t, NH, 2H), 8.29 (t, NH, 1H), 8.14-8.12 (d, 1H), 8.09 (br, 6H), 7.75-7.73 (d, 1H), 6.99-6.97 (d, 1H), 6.67-6.65 (d, 1H), 6.53 (s, 1H), 4.49 (s, 2H), 3.81-3.79 (m, 1H), 3.62-3.60 (m, 4H), 3.59-3.51 (m, 4H), 3.40-3.34 (m, 6H), 3.23-3.16 (br, 2H), 3.08 (s, 3H), 2.93-2.85 (m, 4H), 2.83-2.62 (m, 7H), 2.14-2.09 (m, 3H), 2.05-1.98 (m, 1H), 1.98-1.84 (m, 4H), 1.70-1.65 (m, 4H), 1.55-1.48 (m, 2H), 1.37-1.26 (m, 8H), 1.09-1.04 (m, 1H), 0.82-0.80 (d, 3H), 0.54-0.51 (m, 1H), 0.30-0.19 (m, 2H), −0.05 ˜−0.10 (m, 1H).

### Example 70

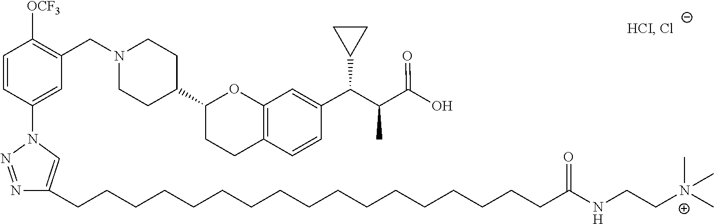 [SMILES: C[C@H](C(=O)O)[C@H](c1ccc2c(c1)O[C@@H](C1CCN(Cc3cc(-n4cc(CCCCCCCCCCCCCCCCCC(=O)NCC[N+](C)(C)C)nn4)ccc3OC(F)(F)F)CC1)CC2)C1CC1]

MS (ESI) m/z 951.4 [M]⁺. ¹H NMR (400 MHZ, DMSO-d₆): δ 8.68 (s, 1H), 8.62 (s, 1H), 8.27 (t, NH, 1H), 8.15-8.12 (dd, 1H), 7.75-7.73 (d, 1H), 6.98-6.96 (d, 1H), 6.67-6.65 (d, 1H), 6.53 (s, 1H), 4.49 (s, 2H), 3.82-3.81 (m, 1H), 3.48-3.46 (m, 4H), 3.38-3.34 (m, 2H), 3.15-3.11 (br, 2H), 3.11 (s, 9H), 2.75-2.62 (m, 5H), 2.12-2.08 (t, 2H), 2.08-1.88 (m, 2H), 1.88-1.75 (m, 4H), 1.75-1.63 (m, 4H), 1.50-1.40 (m, 2H), 1.37-1.24 (m, 26H), 1.11-1.05 (m, 1H), 0.82-0.80 (d, 3H), 0.54-0.51 (m, 1H), 0.30-0.19 (m, 2H), −0.05 ˜−0.10 (m, 1H).

### Example 71

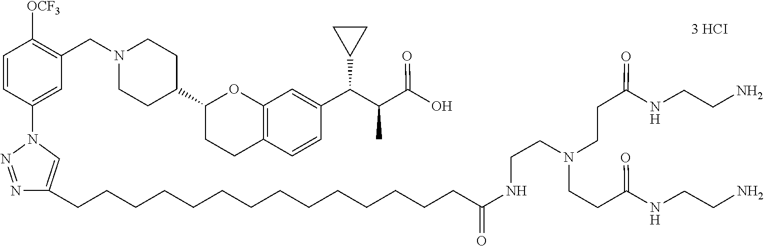 [SMILES: C[C@H](C(=O)O)[C@H](c1ccc2c(c1)O[C@@H](C1CCN(Cc3cc(-n4cc(CCCCCCCCCCCCCCC(=O)NCCN(CCC(=O)NCCN)CCC(=O)NCCN)nn4)ccc3OC(F)(F)F)CC1)CC2)C1CC1]

MS (ESI) m/z 1096.4 [M+1]⁺. ¹H NMR (400 MHZ, DMSO-d₆): δ 8.66 (s, 1H), 8.46 (t, NH, 2H), 8.40 (s, 1H), 8.18 (t, NH, 1H), 8.03-8.00 (d, 1H), 7.99 (br, 6H), 7.60-7.57 (d, 1H), 6.97-6.95 (d, 1H), 6.65-6.63 (d, 1H), 6.51 (s, 1H), 4.44-4.40 (m, 4H), 3.81-3.79 (m, 1H), 3.44-3.38 (m, 2H), 3.37-3.30 (m, 10H), 3.20-3.13 (m, 4H), 2.90-2.86 (m, 4H), 2.74-2.60 (m, 7H), 2.10-2.05 (t, 2H), 2.00-1.80 (m, 8H), 1.75-1.62 (m, 2H), 1.53-1.47 (m, 2H), 1.28-1.22 (m, 20H), 1.09-1.04 (m, 1H), 0.82-0.80 (d, 3H), 0.54-0.51 (m, 1H), 0.30-0.19 (m, 2H), -0.05 ˜−0.10 (m, 1H).

### Example 72

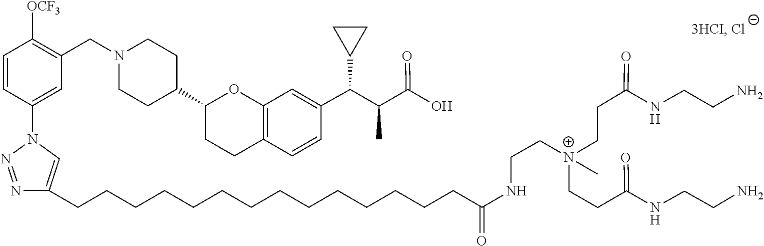 [SMILES: C[C@H](C(=O)O)[C@H](c1ccc2c(c1)O[C@@H](C1CCN(Cc3cc(-n4cc(CCCCCCCCCCCCCCC(=O)NCC[N+](C)(CCC(=O)NCCN)CCC(=O)NCCN)nn4)ccc3OC(F)(F)F)CC1)CC2)C1CC1]

MS (ESI) m/z 1110.4 [M]⁺. ¹H NMR (400 MHZ, DMSO-d₆): δ 8.68 (s, 1H), 8.53 (t, NH, 2H), 8.44 (s, 1H), 8.28-8.27 (t, NH, 1H), 8.04-8.02 (d, 1H), 8.07 (br, 6H), 7.61-7.59 (d, 1H), 6.98-6.96 (d, 1H), 6.67-6.65 (d, 1H), 6.53 (s, 1H), 4.45-4.42 (m, 4H), 3.82-3.80 (m, 1H), 3.63-3.59 (m, 4H), 3,.50-3.30 (m, 8H), 3.20-3.12 (m, 2H), 3.11 (s, 3H), 2.90-2.83 (m, 4H), 2.75-2.62 (m, 7H), 2.12-2.08 (t, 2H), 2.01-1.80 (m, 8H), 1.80-1.60 (m, 2H), 1.52-1.47 (m, 2H), 1.28-1.22 (m, 20H), 1.09-1.04 (m, 1H), 0.82-0.80 (d, 3H), 0.54-0.51 (m, 1H), 0.30-0.19 (m, 2H), −0.05 ˜−0.10 (m, 1H).

### Example 73

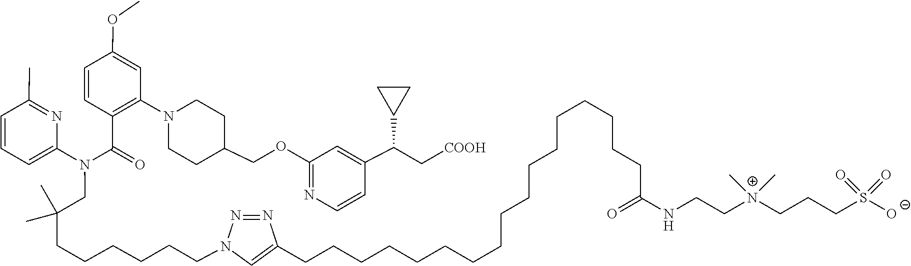 [SMILES: COc1ccc(C(=O)N(CC(C)(C)CCCCCCn2cc(CCCCCCCCCCCCCCCCCC(=O)NCC[N+](C)(C)CCCS(=O)(=O)[O-])nn2)c2cccc(C)n2)c(N2CCC(COc3cc([C@@H](CC(=O)O)C4CC4)ccn3)CC2)c1]

MS (ESI) m/z 1226.5 [M]⁺. ¹H NMR (400 MHZ, DMSO-d₆): δ 8.10 (t, NH, 1H), 8.06-8.04 (d, 1H), 7.80 (s, 1H), 7.26 (br, 1H), 7.12-7.11 (d, 1H), 6.94-6.93 (d, 1H), 6.85-6.83 (d, 1H), 6.74 (s, 1H), 6.53-6.50 (d, 1H), 6.40 (br, 1H), 6.23 (s, 1H), 4.24-4.21 (t, 2H), 4.14-4.12 (d, 2H), 4.08 (br, 2H), 3.69 (s, 3H), 3.46-3.42 (m, 4H), 3.33-3.29 (m, 2H), 3.08 (s, 3H), 2.70-2.68 (d, 2H), 2.59-2.55 (t, 2H), 2.51-2.46 (m, 2H), 2.33 (s, 3H), 2.28-2.22 (m, 1H), 2.10-2.06 (t, 2H), 2.02-1.96 (m, 2H), 1.77-1.63 (m, 5H), 1.57-1.54 (m, 2H), 1.49-1.46 (m, 2H), 1.39 (br, 2H), 1.25 (br, 26H), 1.25-0.94 (m, 9H), 0.75 (s, 6H), 0.53-0.49 (m, 1H), 0.32-0.25 (m, 2H), 0.23-0.18 (m, 1H).

### Example 74

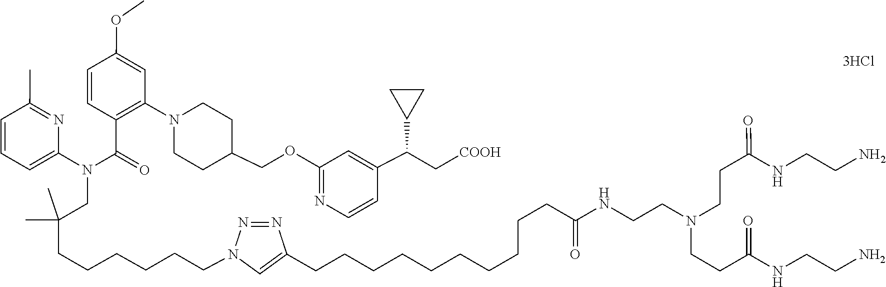 [SMILES: COc1ccc(C(=O)N(CC(C)(C)CCCCCCn2cc(CCCCCCCCCCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

MS (ESI) m/z 1206.4 [M+H]⁺. ¹H NMR (400 MHz, DMSO-d₆): δ 8.40 (t, NH, 2H), 8.14 (t, NH, 1H), 8.06-8.05 (d, 1H), 7.90 (br, NH₃, 6H), 7.81 (s, 1H), 7.28 (br, 1H), 7.12-7.10 (d, 1H), 6.94-6.93 (d, 1H), 6.86-6.84 (d, 1H), 6.73 (s, 1H), 6.54-6.51 (d, 1H), 6.42 (br, 1H), 6.23 (s, 1H), 4.25-4.22 (t, 2H), 4.15-4.13 (d, 2H), 4.10 (br, 2H), 3.70 (s, 3H), 3.43-3.37 (m, 6H), 3.34-3.30 (m, 4H), 3.20-3.18 (m, 2H), 2.91-2.87 (m, 4H), 2.71-2.69 (d, 2H), 2.67-2.62 (m, 4H), 2.60-2.56 (m, 2H), 2.35 (s, 3H), 2.28-2.22 (m, 1H), 2.11-2.07 (t, 2H), 1.74-1.67 (m, 5H), 1.58-1.55 (m, 2H), 1.49-1.46 (m, 2H), 1.39 (br, 2H), 1.25 (br, 12H), 1.25-0.94 (m, 9H), 0.75 (s, 6H), 0.53-0.50 (m, 1H), 0.32-0.25 (m, 2H), 0.23-0.18 (m, 1H).

### Example 75

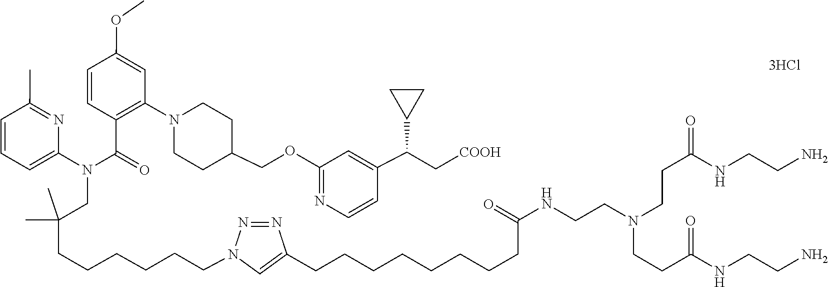 [SMILES: COc1ccc(C(=O)N(CC(C)(C)CCCCCCn2cc(CCCCCCCCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

MS (ESI) m/z 1178.5 [M+H]⁺. ¹H NMR (400 MHZ, DMSO-d₆): δ 8.40 (t, NH, 2H), 8.14 (t, NH, 1H), 8.06-8.04 (d, 1H), 7.88 (br, NH₃, 6H), 7.80 (s, 1H), 7.27 (br, 1H), 7.11-7.09 (d, 1H), 6.93-6.92 (d, 1H), 6.86-6.84 (d, 1H), 6.72 (s, 1H), 6.53-6.51 (d, 1H), 6.43 (br, 1H), 6.23 (s, 1H), 4.25-4.21 (t, 2H), 4.14-4.12 (d, 2H), 4.09 (br, 2H), 3.69 (s, 3H), 3.43-3.37 (m, 6H), 3.34-3.29 (m, 4H), 3.17 (br, 2H), 2.89-2.86 (m, 4H), 2.70-2.68 (d, 2H), 66-2.62 (m, 4H), 2.60-2.56 (m, 2H), 2.34 (s, 3H), 2.27-2.21 (m, 1H), 2.11-2.07 (t, 2H), 1.71-1.66 (m, 5H), 1.57-1.55 (m, 2H), 1.49-1.46 (m, 2H), 1.39 (br, 2H), 1.25 (br, 8H), 1.25-0.94 (m, 9H), 0.75 (s, 6H), 0.53-0.49 (m, 1H), 0.32-0.25 (m, 2H), 0.23-0.18 (m, 1H).

### Example 76

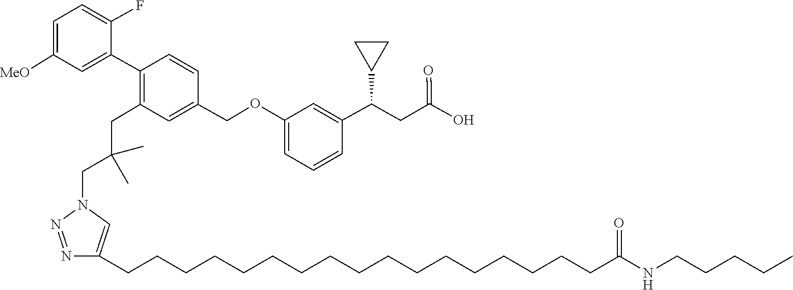 [SMILES: CCCCCNC(=O)CCCCCCCCCCCCCCCCCc1cn(CC(C)(C)Cc2cc(COc3cccc([C@@H](CC(=O)O)C4CC4)c3)ccc2-c2cc(OC)ccc2F)nn1]

MS (ESI) m/z 909.4 [M+H]⁺. ¹H NMR (400 MHz, DMSO-d₆): δ 7.71 (br, NH, 1H), 7.63 (s, 1H), 7.44-. 40 (m, 2H), 7.23-7.12 (m, 3H), 6.91-6.77 (m, 5H), 5.14 (s, 2H), 3.98 (s, 2H), 3.73 (s, 3H), 3.01-2.99 (m, 2H), 2.72-2.55 (m, 6H), 2.26-2.23 (m, 1H), 2.04-2.00 (m, 2H), 1.54 (br, 2H), 1.46 (br, 2H), 1.36-1.33 (m, 2H), 1.25-1.13 (m, 30H), 0.99-0.97 (m, 1H), 0.85-0.83 (m, 3H), 0.52 (br, 7H), 0.33-0.23 (m, 2H), 0.12-0.10 (m, 1H).

### Example 77

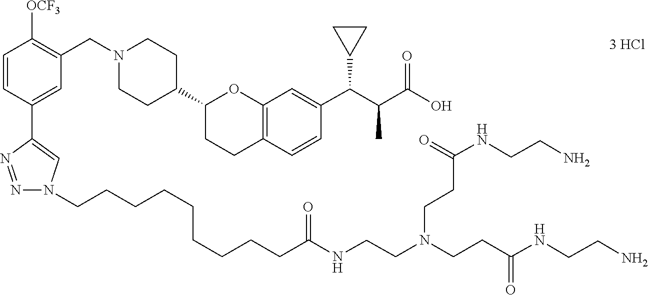 [SMILES: C[C@H](C(=O)O)[C@H](c1ccc2c(c1)O[C@@H](C1CCN(Cc3cc(-c4cn(CCCCCCCCCC(=O)NCCN(CCC(=O)NCCN)CCC(=O)NCCN)nn4)ccc3OC(F)(F)F)CC1)CC2)C1CC1]

MS (ESI) m/z 1025.4 [M+H]⁺. ¹H NMR (400 MHZ, DMSO-d₆): δ 8.66 (s, 1H), 8.43 (t, NH, 2H), 8.40 (s, 1H), 8.17 (t, NH, 1H), 8.03-7.99 (d, 1H), 7.98 (br, 6H), 7.60-7.58 (d, 1H), 6.97-6.95 (d, 1H), 6.66-6.64 (d, 1H), 6.52 (s, 1H), 4.44-4.41 (m, 4H), 3.82-3.79 (m, 1H), 3.44-3.30 (m, 12H), 3.20-3.13 (m, 4H), 2.90-2.86 (m, 4H), 2.74-2.60 (m, 7H), 2.11-2.07 (t, 2H), 2.00-1.80 (m, 8H), 1.75-1.62 (m, 2H), 1.53-1.47 (m, 2H), 1.28-1.22 (m, 10H), 1.09-1.04 (m, 1H), 0.82-0.80 (d, 3H), 0.54-0.51 (m, 1H), 0.30-0.19 (m, 2H), −0.05 ˜−0.10 (m, 1H).

### Example 78

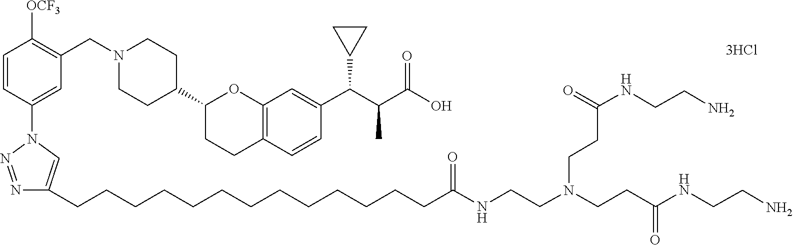 [SMILES: C[C@H](C(=O)O)[C@H](c1ccc2c(c1)O[C@@H](C1CCN(Cc3cc(-n4cc(CCCCCCCCCCCCCC(=O)NCCN(CCC(=O)NCCN)CCC(=O)NCCN)nn4)ccc3OC(F)(F)F)CC1)CC2)C1CC1]

MS (ESI) m/z 1081.4 [M+H]⁺. ¹H NMR (400 MHZ, DMSO-d₆): δ 8.6 (s, 1H), 8.53 (s, 1H), 8.43 (t, NH, 2H), 8.15 (t, NH, 1H), 8.11-8.09 (d, 1H), 7.95 (br, 6H), 7.73-7.71 (d, 1H), 6.97-6.95 (d, 1H), 6.66-6.64 (d, 1H), 6.52 (s, 1H), 4.47 (s, 2H), 3.82-3.80 (m, 1H), 3.50-3.30 (m, 12H), 3.23-3.13 (m, 4H), 2.93-2.87 (m, 4H), 2.80-2.63 (m, 9H), 2.11-2.07 (m, 2H), 2.05-1.98 (m, 2H), 1.98-1.84 (m, 4H), 1.70-1.65 (m, 4H), 1.55-1.48 (m, 2H), 1.37-1.26 (m, 18H), 1.09-1.04 (m, 1H), 0.82-0.80 (d, 3H), 0.54-0.51 (m, 1H), 0.30-0.19 (m, 2H), -0.05 ˜−0.10 (m, 1H).

### Example 79

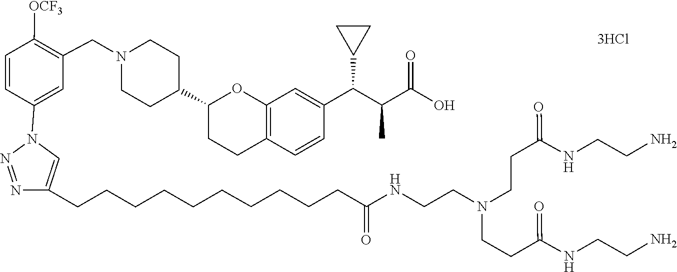 [SMILES: C[C@H](C(=O)O)[C@H](c1ccc2c(c1)O[C@@H](C1CCN(Cc3cc(-n4cc(CCCCCCCCCCC(=O)NCCN(CCC(=O)NCCN)CCC(=O)NCCN)nn4)ccc3OC(F)(F)F)CC1)CC2)C1CC1]

MS (ESI) m/z 1039.5 [M+H]⁺. ¹H NMR (400 MHZ, DMSO-d₆): δ 8.66 (s, 1H), 8.58 (s, 1H), 8.47 (t, NH, 2H), 8.19 (t, NH, 1H), 8.13-8.10 (d, 1H), 8.02 (br, 6H), 7.74-7.71 (d, 1H), 6.97-6.95 (d, 1H), 6.66-6.64 (d, 1H), 6.52 (s, 1H), 4.47 (s, 2H), 3.82-3.78 (m, 1H), 3.50-3.30 (m, 12H), 3.23-3.13 (m, 4H), 2.93-2.87 (m, 4H), 2.75-2.63 (m, 9H), 2.12-2.08 (t, 2H), 2.05-1.98 (m, 2H), 1.98-1.84 (m, 4H), 1.70-1.65 (m, 4H), 1.55-1.48 (m, 2H), 1.37-1.26 (m, 12H), 1.09-1.04 (m, 1H), 0.82-0.80 (d, 3H), 0.54-0.51 (m, 1H), 0.30-0.19 (m, 2H), -0.05 ˜−0.10 (m, 1H).

### Example 80

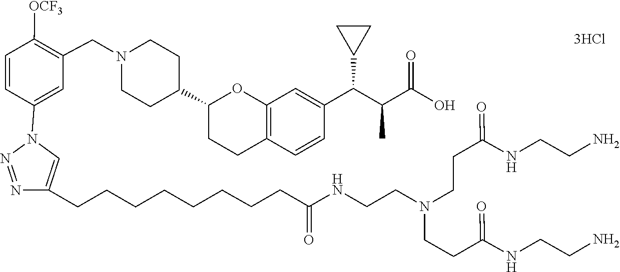 [SMILES: C[C@H](C(=O)O)[C@H](c1ccc2c(c1)O[C@@H](C1CCN(Cc3cc(-n4cc(CCCCCCCCC(=O)NCCN(CCC(=O)NCCN)CCC(=O)NCCN)nn4)ccc3OC(F)(F)F)CC1)CC2)C1CC1]

MS (ESI) m/z 1011.4 [M+H]⁺. ¹H NMR (400 MHZ, DMSO-d₆): δ 8.66 (s, 1H), 8.60 (s, 1H), 8.48 (t, NH, 2H), 8.20 (t, NH, 1H), 8.12-8.10 (d, 1H), 8.05 (br, 6H), 7.72-7.70 (d, 1H), 6.97-6.95 (d, 1H), 6.65-6.63 (d, 1H), 6.52 (s, 1H), 4.46 (s, 2H), 3.80-3.78 (m, 1H), 3.50-3.30 (m, 12H), 3.33-3.13 (m, 4H), 2.91-2.86 (m, 4H), 2.77-2.65 (m, 9H), 2.12-2.08 (t, 2H), 2.05-1.98 (m, 2H), 1.98-1.84 (m, 4H), 1.70-1.65 (m, 4H), 1.55-1.48 (m, 2H), 1.37-1.26 (m, 8H), 1.09-1.04 (m, 1H), 0.82-0.80 (d, 3H), 0.54-0.51 (m, 1H), 0.30-0.19 (m, 2H), −0.05 ˜−0.10 (m, 1H).

### Example 81

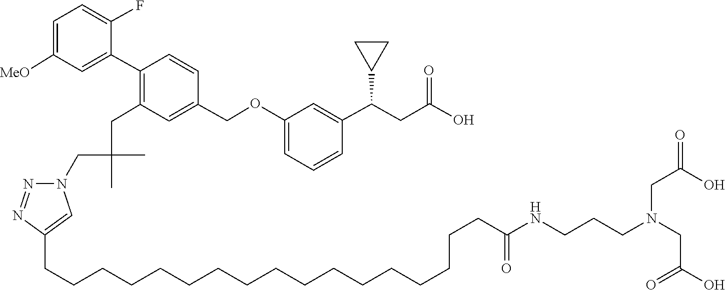 [SMILES: COc1ccc(F)c(-c2ccc(COc3cccc([C@@H](CC(=O)O)C4CC4)c3)cc2CC(C)(C)Cn2cc(CCCCCCCCCCCCCCCCCC(=O)NCCCN(CC(=O)O)CC(=O)O)nn2)c1]

MS (ESI) m/z 1012.4 [M+H]⁺. ¹H NMR (400 MHZ, DMSO-d₆): δ 7.80 (br, NH, 1H), 7.62 (s, 1H), 7.44-7.40 (m, 2H), 7.23-7.12 (m, 3H), 6.91-6.77 (m, 5H), 5.13 (s, 2H), 3.97 (s, 2H), 3.76 (s, 4H), 3.73 (s, 3H), 3.12-3.05 (m, 2H), 2.95-2.90 (m, 2H), 2.72-2.55 (m, 6H), 2.28-2.23 (m, 1H), 2.07-1.97 (m, 2H), 1.65-1.61 (m, 2H), 1.56-1.52 (m, 2H), 1.46-1.41 (m, 2H), 1.35-1.13 (m, 26H), 0.99-0.97 (m, 1H), 0.52 (br, 7H), 0.33-0.23 (m, 2H), 0.12-0.10 (m, 1H).

### Example 82

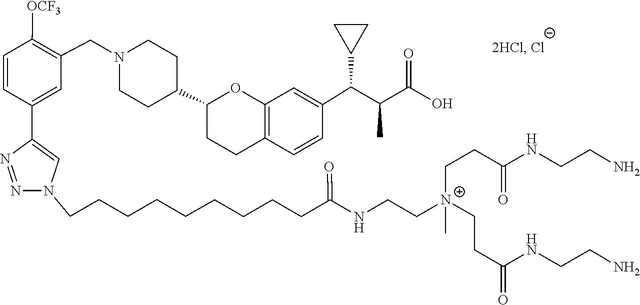 [SMILES: C[C@H](C(=O)O)[C@H](c1ccc2c(c1)O[C@@H](C1CCN(Cc3cc(-c4cn(CCCCCCCCCC(=O)NCC[N+](C)(CCC(=O)NCCN)CCC(=O)NCCN)nn4)ccc3OC(F)(F)F)CC1)CC2)C1CC1]

MS (ESI) m/z 1039.5 [M]⁺. ¹H NMR (400 MHZ, DMSO-d₆): δ 8.66 (s, 1H), 8.48 (t, NH, 2H), 8.39 (s, 1H), 8.22 (t, NH, 1H), 8.00 (d, 1H), 8.00-7.94 (br, 6H), 7.59-7.56 (d, 1H), 6.97-6.95 (d, 1H), 6.65-6.64 (d, 1H), 6.52 (s, 1H), 4.44-4.40 (m, 4H), 3.83-3.78 (m, 1H), 3.61-3.58 (m, 4H), 3.50-3.25 (m, 8H), 3.13-3.05 (m, 2H), 3.00 (s, 3H), 2.89-2.86 (m, 4H), 2.73-2.63 (m, 7H), 2.11-2.07 (t, 2H), 2.01-1.80 (m, 8H), 1.80-1.60 (m, 2H), 1.52-1.47 (m, 2H), 1.28-1.22 (m, 10H), 1.09-1.04 (m, 1H), 0.82-0.80 (d, 3H), 0.54-0.51 (m, 1H), 0.30-0.19 (m, 2H), −0.05 ˜−0.10 (m, 1H).

### Example 83

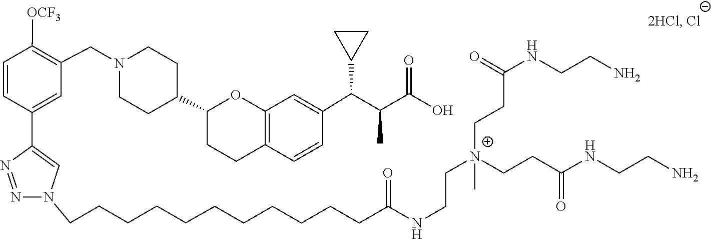 [SMILES: C[C@H](C(=O)O)[C@H](c1ccc2c(c1)O[C@@H](C1CCN(Cc3cc(-c4cn(CCCCCCCCCCCC(=O)NCC[N+](C)(CCC(=O)NCCN)CCC(=O)NCCN)nn4)ccc3OC(F)(F)F)CC1)CC2)C1CC1]

MS (ESI) m/z 1067.5 [M]⁺. ¹H NMR (400 MHZ, DMSO-d₆): δ 8.67 (s, 1H), 8.52 (t, NH, 2H), 8.47 (s, 1H), 8.23 (t, NH, 1H), 8.02 (d, 1H), 8.07-8.02 (br, 6H), 7.61-7.59 (d, 1H), 6.97-6.95 (d, 1H), 6.67-6.64 (d, 1H), 6.51 (s, 1H), 4.44-4.40 (m, 4H), 3.83-3.78 (m, 1H), 3.63-3.59 (m, 4H), 3.47-3.42 (m, 2H), 3.37-3.25 (m, 6H), 3.16-3.10 (m, 2H), 3.05 (s, 3H), 2.89-2.85 (m, 4H), 2.73-2.63 (m, 7H), 2.11-2.06 (t, 2H), 2.01-1.80 (m, 8H), 1.80-1.60 (m, 2H), 1.52-1.47 (m, 2H), 1.28-1.22 (m, 14H), 1.09-1.04 (m, 1H), 0.82-0.80 (d, 3H), 0.54-0.51 (m, 1H), 0.30-0.19 (m, 2H), −0.05 ˜−0.10 (m, 1H).

### Example 84

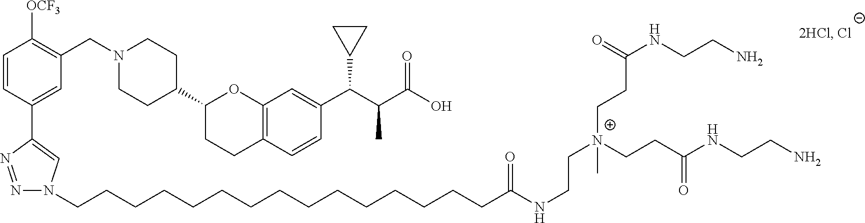 [SMILES: C[C@H](C(=O)O)[C@H](c1ccc2c(c1)O[C@@H](C1CCN(Cc3cc(-c4cn(CCCCCCCCCCCCCCCC(=O)NCC[N+](C)(CCC(=O)NCCN)CCC(=O)NCCN)nn4)ccc3OC(F)(F)F)CC1)CC2)C1CC1]

MS (ESI) m/z 562.4 (M+H)/2]⁺. ¹H NMR (400 MHZ, DMSO-d₆): δ 8.67 (s, 1H), 8.50 (t, NH, 2H), 8.45 (s, 1H), 8.23-8.20 (t, NH, 1H), 8.00-7.96 (d, 1H), 7.96 (br, 6H), 7.61-7.59 (d, 1H), 6.98-6.96 (d, 1H), 6.67-6.65 (d, 1H), 6.51 (s, 1H), 4.44-4.40 (m, 4H), 3.82-3.78 (m, 1H), 3.62-3.56 (m, 4H), 3,.46-3.44 (m, 2H), 3.37-3.25 (m, 6H), 3.15-3.13 (m, 2H), 3.06 (s, H), 2.90-2.85 (m, 4H), 2.73-2.67 (m, 7H), 2.11-2.06 (t, 2H), 2.01-1.80 (m, 8H), 1.80-1.60 (m, 2H), 1.52-1.47 (m, 2H), 1.28-1.22 (m, 22H), 1.09-1.04 (m, 1H), 0.82-0.80 (d, 3H), 0.54-0.51 (m, 1H), 0.30-0.19 (m, 2H), −0.05 ˜−0.10 (m, 1H).

### Example 85

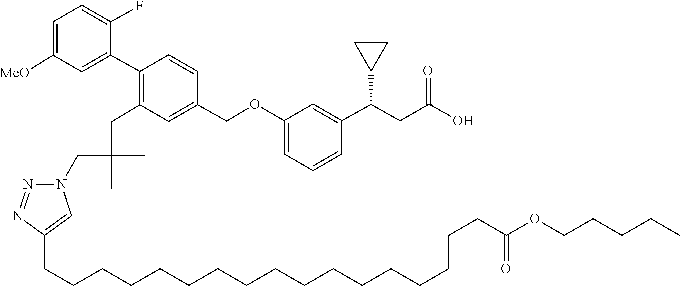 [SMILES: CCCCCOC(=O)CCCCCCCCCCCCCCCCCc1cn(CC(C)(C)Cc2cc(COc3cccc([C@@H](CC(=O)O)C4CC4)c3)ccc2-c2cc(OC)ccc2F)nn1]

¹H NMR (400 MHZ, DMSO-d₆): δ 7.62 (s, 1H), 7.42 (s, 1H), 7.40-7.38 (d, 1H), 7.23-7.20 (d, 1H), 7.17-7.12 (m, 2H), 6.95-6.91 (m, 1H), 6.83 (s, 1H), 6.81-6.74 (m, 3H), 5.13 (s, 2H), 4.00-3.96 (m, 4H), 3.73 (s, 3H), 2.78-2.67 (m, 3H), 2.75-2.51 (m, 3H), 2.27-2.23 (m, 1H), 2.27-2.24 (t, 2H), 1.55-1.42 (m, 6H), 1.30-1.15 (m, 30H), 1.01-0.97 (m, 1H), 0.87-0.84 (t, 3H), 0.56-0.44 m, 7H), 0.33-0.23 (m, 2H), 0.12-0.10 (m, 1H).

### Example 86

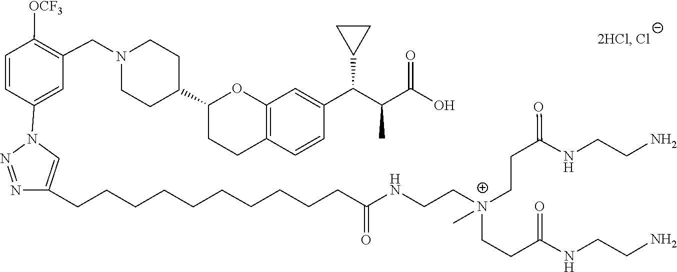 [SMILES: C[C@H](C(=O)O)[C@H](c1ccc2c(c1)O[C@@H](C1CCN(Cc3cc(-n4cc(CCCCCCCCCCC(=O)NCC[N+](C)(CCC(=O)NCCN)CCC(=O)NCCN)nn4)ccc3OC(F)(F)F)CC1)CC2)C1CC1]

MS (ESI) m/z 1054.4 [(M+H)/2]⁺. ¹H NMR (400 MHZ, DMSO-d₆): δ 8.72 (t, NH, 2H), 8.62 (m, 2H), 8.34 (s, 1H), 8.16 (br, 6H), 8.15-8.13 (d, 1H), 7.75-7.73 (d, 1H), 6.98-6.96 (d, 1H), 6.67-6.65 (d, 1H), 6.54 (s, 1H), 4.49 (s, 2H), 3.81-3.79 (m, 1H), 3.64-3.61 (m, 4H), 358-3.50 (m, 4H), 3.36-3.34 (m, 6H), 3.17-3.15 (br, 2H), 3.09 (s, 3H), 2.90-2.887 (m, 4H), 2.74-2.63 (m, 9H), 2.13-2.07 (m, 3H), 2.07-1.98 (m, 1H), 1.96-1.83 (m, 4H), 1.68-1.62 (m, 4H), 1.55-1.48 (m, 2H), 1.37-1.26 (m, 8H), 1.09-1.04 (m, 1H), 0.82-0.80 (d, 3H), 0.54-0.51 (m, 1H), 0.30-0.19 (m, 2H), −0.05 ˜−0.10 (m, 1H).

### Example 87

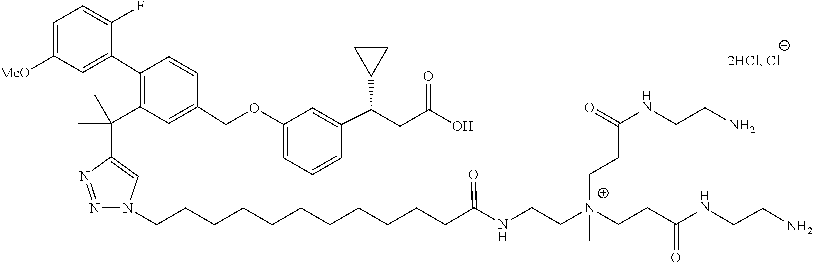 [SMILES: COc1ccc(F)c(-c2ccc(COc3cccc([C@@H](CC(=O)O)C4CC4)c3)cc2C(C)(C)c2cn(CCCCCCCCCCCC(=O)NCC[N+](C)(CCC(=O)NCCN)CCC(=O)NCCN)nn2)c1]

MS (ESI) m/z 1012.3 [M+H]⁺. ¹H NMR (400 MHZ, DMSO-d₆): δ 8.54-8.52 (t, NH, 2H), 8.25-8.23 (t, 1H), 8.07 (br, NH₃, 6H), 7.69 (s, 1H), 7.35-7.34 (d, 1H), 7.31 (s, 1H), 7.24-7.21 (m, 1H), 6.90-6.78 (m, 6H), 6.95-5.93 (m, 1H), 5.14 (s, 3H), 4.15-4.00 (m, 2H), 3.63-3.60 (m, 4H), 3.59 (s, 3H), 3.47-3.44 (m, 2H), 3.34-3.30 (m, 6H), 3.06 (s, 3H), 2.90-2.85 (m, 4H), 2.73-2.64 (m, 6H), 2.28-2.24 (m, 1H), 2.12-2.08 (t, 2H), 1.73-1.68 (m, 2H), 1.59 (s, 3H), 1.48-1.44 (m, 2H), 1.44 (s, 3H), 1.30-1.19 (m, 14H), 0.99-0.97 (m, 1H), 0.52-0.48 (m, 1H), 0.33-0.23 (m, 2H), 0.12-0.10 (m, 1H).

### Example 88

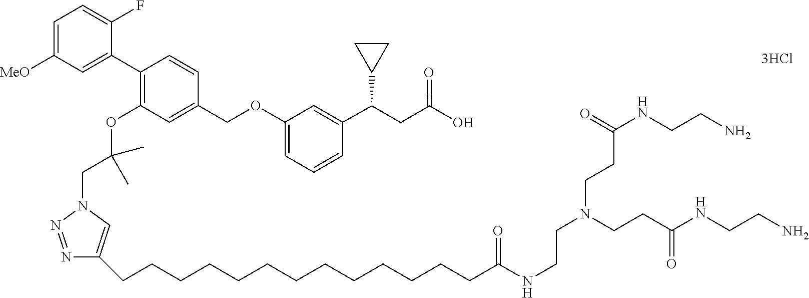 [SMILES: COc1ccc(F)c(-c2ccc(COc3cccc([C@@H](CC(=O)O)C4CC4)c3)cc2OC(C)(C)Cn2cc(CCCCCCCCCCCCCC(=O)NCCN(CCC(=O)NCCN)CCC(=O)NCCN)nn2)c1]

MS (ESI) m/z 1056.3 [M+H]⁺. ¹H NMR (400 MHZ, DMSO-d₆): δ 8.43-8.41 (t, NH, 2H), 8.16 (t, NH, 1H), 7.94 (br, 6H), 7.32-7.30 (d, 1H), 7.24-7.16 (m, 4H), 7.12 (s, 1H), 6.98-6.94 (m, 1H), 6.91-6.88 (m, 2H), 6.85-6.83 (m, 2H), 5.13 (s, 2H), 4.30 (s, 2H), 3.76 (s, 3H), 3.50-3.30 (m, 10H), 3.20-3.13 (m, 2H), 2.89-2.86 (m, 4H), 2.69-2.61 (m, 6H), 2.52-2.48 (t, 2H), 2.26-2.23 (m, 1H), 2.11-2.07 (t, 2H), 1.51-1.46 (m, 4H), 1.30-1.20 (m, 18H), 1.07 (s, 6H), 0.99-0.97 (m, 1H), 0.52-0.48 (m, 1H), 0.33-0.23 (m, 2H), 0.12-0.10 (m, 1H).

### Example 89

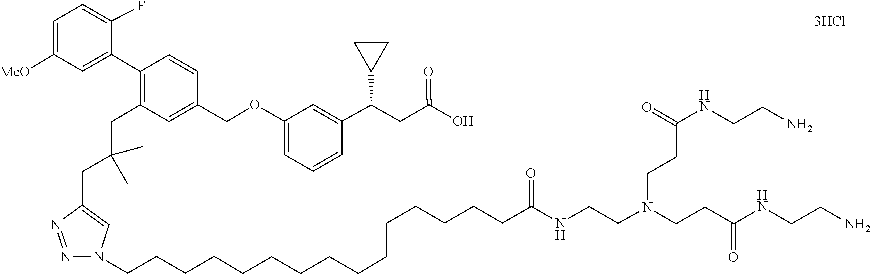 [SMILES: COc1ccc(F)c(-c2ccc(COc3cccc([C@@H](CC(=O)O)C4CC4)c3)cc2CC(C)(C)Cc2cn(CCCCCCCCCCCCCCCC(=O)NCCN(CCC(=O)NCCN)CCC(=O)NCCN)nn2)c1]

MS (ESI) m/z 1082.4 [M+H]⁺. ¹H NMR (400 MHZ, DMSO-d₆): δ 8.42 (t, NH, 2H), 8.15 (t, NH, 1H), 7.93-7.85 (br s, 6H), 7.70 (s, 1H), 7.45 (s, 1H), 7.39-7.37 (d, 1H), 7.22-7.13 (m, 3H), 6.96-6.91 (m, 2H), 6.86-6.78 (m, 3H), 5.12 (s, 2H), 4.26-4.23 (t, 2H), 3.74 (s, 3H), 3.45-3.30 (m, 10H), 3.20-3.15 (m, 2H), 2.88-2.86 (m, 4H), 2.70-2.55 (m, 7H), 2.52-2.49 (m, 1H), 2.34 (s, 2H), 2.26-2.22 (m, 1H), 2.11-2.07 (t, 2H), 1.76-1.69 (m, 2H), 1.52-1.47 (m, 2H), 1.30-1.13 (m, 22H), 0.99-0.97 (m, 1H), 0.55-0.50 (m, 6H), 0.52-0.48 (br, 1H), 0.33-0.23 (m, 2H), 0.12-0.10 (m, 1H).

### Example 90

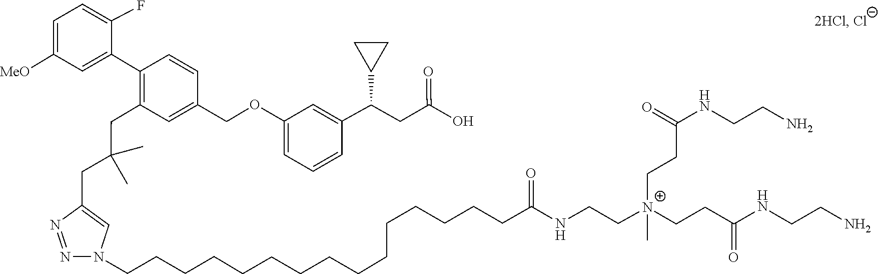 [SMILES: COc1ccc(F)c(-c2ccc(COc3cccc([C@@H](CC(=O)O)C4CC4)c3)cc2CC(C)(C)Cc2cn(CCCCCCCCCCCCCCCC(=O)NCC[N+](C)(CCC(=O)NCCN)CCC(=O)NCCN)nn2)c1]

MS (ESI) m/z 548.9 [(M+H)/2]⁺. ¹H NMR (400 MHZ, DMSO-d₆): δ 8.52 (t, NH, 2H), 8.23 (t, NH, 1H), 8.05-8.00 (br, 6H), 7.71 (s, 1H), 7.45 (s, 1H), 7.40-7.37 (d, 1H), 7.22-7.1 (m, 3H), 6.96-6.91 (m, 2H), 6.86-6.78 (m, 3H), 5.12 (s, 2H), 4.26-4.23 (t, 2H), 3.74 (s, 3H), 3.63-3.58 (m, 4H), 3.50-3.45 (m, 2H), 3.34-3.30 (m, 6H), 3.06 (s, 3H), 2.90-2.85 (m, 4H), 2.73-2.70 (m, 5H), 2.63-2.60 (m, 2H), 2.52-2.49 (m, 1H), 2.34 (s, 2H), 2.26-2.22 (m, 1H), 2.11-2.07 (t, 2H), 1.78-1.70 (m, 2H), 1.78-1.70 (m, 2H), 1.30-1.13 (m, 22H), 0.99-. 97 (m, 1H), 0.55-0.50 (m, 6H), 0.52-0.48 (br, 1H), 0.33-0.23 (m, 2H), 0.12-0.10 (m, 1H).

### Example 91

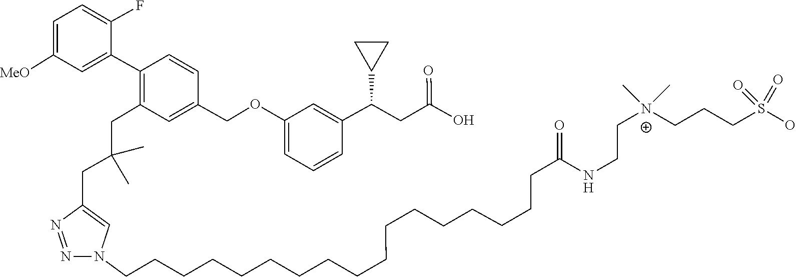 [SMILES: COc1ccc(F)c(-c2ccc(COc3cccc([C@@H](CC(=O)O)C4CC4)c3)cc2CC(C)(C)Cc2cn(CCCCCCCCCCCCCCCCCC(=O)NCC[N+](C)(C)CCCS(=O)(=O)O)nn2)c1]

MS (ESI) m/z 1032.4 [(M+H)]⁺. ¹H NMR (400 MHZ, DMSO-d₆): δ 8.09 (t, NH, 1H), 7.70 (s, 1H), 7.45 (s, 1H), 7.39-7.37 (d, 1H), 7.22-7.14 (m, 3H), 6.96-6.92 (m, 2H), 6.86-6.78 (m, 3H), 5.12 (s, 2H), 4.26-4.23 (t, 2H), 3.74 (s, 3H), 3.46-3.42 (m, 4H), 3.32-3.3.29 (m, 2H), 3.04 (s, 6H), 2.72-2.60 (m, 3H), 2.54-2.45 (m, 3H), 2.34 (s, 2H), 2.26-2.22 (m, 1H), 2.11-2.07 (t, 2H), 2.00-1.95 (m, 2H), 1.78-1.72 (m, 2H), 1.49-1.44 (m, 2H), 1.30-1.13 (m, 26H), 0.99-0.97 (m, 1H), 0.55-0.50 (m, 6H), 0.52-0.48 (br, 1H), 0.33-0.23 (m, 2H), 0.12-0.10 (m, 1H).

### Example 92

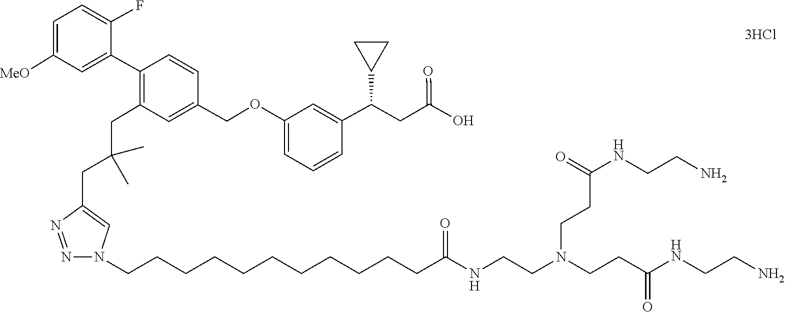 [SMILES: COc1ccc(F)c(-c2ccc(COc3cccc([C@@H](CC(=O)O)C4CC4)c3)cc2CC(C)(C)Cc2cn(CCCCCCCCCCCC(=O)NCCN(CCC(=O)NCCN)CCC(=O)NCCN)nn2)c1 ; "3HCl"]

MS (ESI) m/z 1026.4 [(M+H)]⁺. ¹H NMR (400 MHZ, DMSO-d₆): δ 8.44 (t, NH, 2H), 8.15 (t, NH, 1H), 8.01-7.93 (br s, 6H), 7.70 (s, 1H), 7.45 (s, 1H), 7.40-7.38 (d, 1H), 7.22-7.14 (m, 3H), 6.96-6.92 (m, 2H), 6.86-6.78 (m, 3H), 5.12 (s, 2H), 4.26-4.23 (t, 2H), 3.74 (s, 3H), 3.45-3.30 (m, 10H), 3.18-3.15 (m, 2H), 2.88-2.86 (m, 4H), 2.70-2.55 (m, 7H), 2.52-2.49 (m, 1H), 2.34 (s, 2H), 2.28-2.22 (m, 1H), 2.11-2.07 (t, 2H), 1.77-1.72 (m, 2H), 1.52-1.47 (m, 2H), 1.30-1.13 (m, 14H), 0.99-0.97 (m, 1H), 0.55-0.50 (m, 6H), 0.52-0.48 (br, 1H), 0.33-0.23 (m, 2H), 0.12-0.10 (m, 1H).

### Example 93

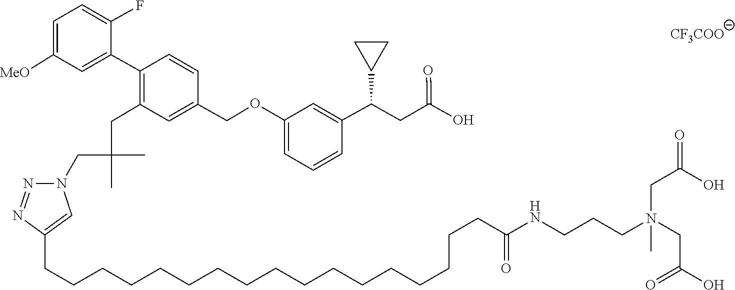

MS (ESI) m/z 1027.4 [M]⁺. ¹H NMR (400 MHZ, DMSO-d₆): δ 7.87 (t, NH, 1H), 7.62 (s, 1H), 7.44-7.40 (m, 2H), 7.41-7.39 (d, 1H), 7.24-7.14 (m, 3H), 7.12 (s, 1H), 6.99 (s, 1H), 6.95-6.77 (m, 5H), 5.14 (s, 2H), 4.44 (s, 4H), 3.98 (s, 2H), 3.73 (s, 3H), 3.66-3.62 (m, 2H), 3.30 (s, 3H), 3.10-3.06 (m, 2H), 2.72-2.55 (m, 6H), 2.28-2.23 (m, 1H), 2.07-1.97 (m, 2H), 1.65-1.61 (m, 2H), 1.56-1.41 (m, 2H), 1.35-1.13 (m, 26H), 0.99-0.97 (m, 1H), 0.52 (br, 7H), 0.33-0.23 (m, 2H), 0.12-0.10 (m, 1H).

### Example 94

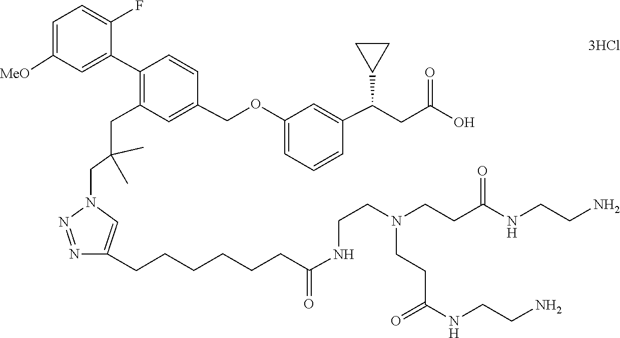 [SMILES: COc1ccc(F)c(-c2ccc(COc3cccc([C@@H](CC(=O)O)C4CC4)c3)cc2CC(C)(C)Cn2cc(CCCCCCC(=O)NCCN(CCC(=O)NCCN)CCC(=O)NCCN)nn2)c1]

MS (ESI) m/z 956.4 [M+H]⁺. ¹H NMR (400 MHZ, DMSO-d₆): δ 8.48 (t, NH, 2H), 8.21 (t, NH, 1H), 8.02 (br, 6H), 7.64 (s, 1H), 7.44 (s, 1H), 7.41-7.39 (d, 1H), 7.23-7.13 (m, 3H), 6.95-6.77 (m, 5H), 5.14 (s, 2H), 3.99 (s, 2H), 3.73 (s, 3H), 3.48-3.32 (m, 10H), 3.19-3.12 (m, 2H), 2.89-2.85 (m, 4H), 2.73-2.67 (m, 7H), 2.63-2.58 (m, 3H), 2.27-2.23 (m, 1H), 2.13-2.09 (t, 2H), 1.56-1.44 (m, 4H), 1.30-1.20 (m, 4H), 1.01-0.97 (m, 1H), 0.52 (br, 7H), 0.33-0.23 (m, 2H), 0.12-0.10 (m, 1H).

### Example 95

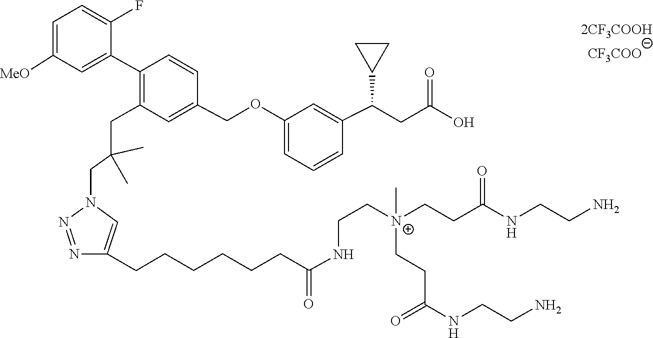 [SMILES: COc1ccc(F)c(-c2ccc(COc3cccc([C@@H](CC(=O)O)C4CC4)c3)cc2CC(C)(C)Cn2cc(CCCCCCC(=O)NCC[N+](C)(CCC(=O)NCCN)CCC(=O)NCCN)nn2)c1 ; "CF3COO" ; "2CF3COOH"]

MS (ESI) m/z 970.3 [M]⁺. ¹H NMR (400 MHZ, DMSO-d₆): δ 8.60 (t, NH, 2H), 8.32 (t, NH, 1H), 8.18 (br, 6H), 7.65 (s, 1H), 7.44 (s, 1H), 7.41-7.39 (d, 1H), 7.23-7.12 (m, 3H), 6.95-6.91 (m, 2H), 6.86-6.82 (m, 2H), 6.79-6.76 (m, 1H), 5.13 (s, 2H), 3.99 (s, 2H), 3.73 (s, 3H), 3.64-3.60 (m, 4H), 3.48-3.46 (m, 2H), 3.34-3.33 (m, 6H), 3.07 (s, 3H), 2.90-2.85 (m, 4H), 2.74-2.70 (m, 5H), 2.67-2.62 (t, 2H), 2.57-2.53 (t, 3H), 2.27-2.22 (m, 1H), 2.13-2.09 (t, 2H), 1.55-1.47 (m, 4H), 1.28-1.23 (m, 4H), 1.00-0.96 (m, 1H), 0.56-0.47 (br, 7H), 0.33-0.23 (m, H), 0.12-0.10 (m, 1H).

### Example 96

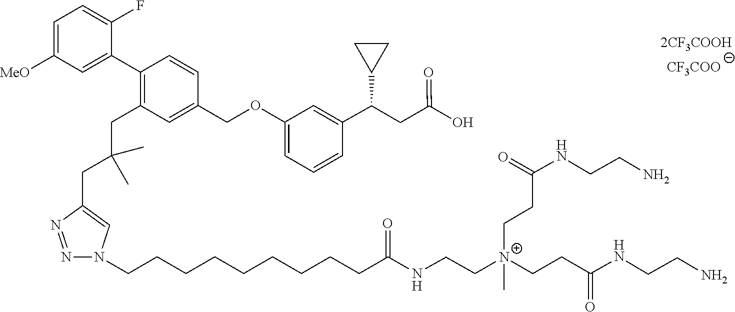 [SMILES: COc1ccc(F)c(-c2ccc(COc3cccc([C@@H](CC(=O)O)C4CC4)c3)cc2CC(C)(C)Cc2cn(CCCCCCCCCC(=O)NCC[N+](C)(CCC(=O)NCCN)CCC(=O)NCCN)nn2)c1 ; "CF3COO" ; "2CF3COOH"]

MS (ESI) m/z 1013.4 [M]⁺. ¹H NMR (400 MHZ, DMSO-d₆): δ 8.57 (t, NH, 2H), 8.28 (t, NH, 1H), 8.13 (br, 6H), 7.70 (s, 1H), 7.45 (s, 1H), 7.39-7.37 (d, 1H), 7.25-7.13 (m, 3H), 6.96-6.91 (m, 2H), 6.86-6.77 (m, 3H), 5.12 (s, 2H), 4.26-4.23 (t, 2H), 3.74 (s, 3H), 3.63-3.57 (m, 4H), 3.50-3.45 (m, 2H), 3.36-3.31 (m, 6H), 3.07 (s, 3H), 2.90-2.86 (m, 4H), 2.74-2.69 (m, 5H), 2.67-2.64 (m, 2H), 2.60-2.54 (m, 1H), 2.34 (s, 2H), 2.28-2.24 (m, 1H), 2.11-2.07 (t, 2H), 1.77-1.74 (m, 2H), 1.47 (br, 2H), 1.30-1.17 (m, 10H), 0.99-0.97 (m, 1H), 0.55-0.50 (m, 6H), 0.52-0.48 (br, 1H), 0.33-0.23 (m, 2H), 0.12-0.10 (m, 1H).

### Example 97

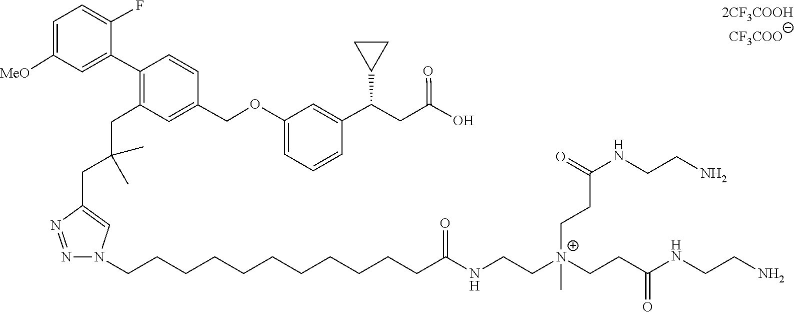 [SMILES: COc1ccc(F)c(-c2ccc(COc3cccc([C@@H](CC(=O)O)C4CC4)c3)cc2CC(C)(C)Cc2cn(CCCCCCCCCCCC(=O)NCC[N+](C)(CCC(=O)NCCN)CCC(=O)NCCN)nn2)c1 ; "CF3COO" ; "2CF3COOH"]

MS (ESI) m/z 1041.4 [M]⁺. ¹H NMR (400 MHZ, DMSO-d₆): δ 8.52 (t, NH, 2H), 8.49 (t, NH, 1H), 8.07 (br, 6H), 7.70 (s, 1H), 7.45 (s, 1H), 7.39-7.37 (d, 1H), 7.22-7.14 (m, 3H), 6.96-6.91 (m, 2H), 6.86-6.77 (m, 3H), 5.12 (s, 2H), 4.26-4.23 (t, 2H), 3.74 (s, 3H), 3.63-3.59 (m, 4H), 3.45-3.40 (m, 2H), 3.36-3.31 (m, 6H), 3.06 (s, 3H), 2.90-2.85 (m, 4H), 2.73-2.68 (m, 5H), 2.67-2.63 (m, 2H), 2.55-2.50 (m, 1H), 2.34 (s, 2H), 2.26-2.22 (m, 1H), 2.11-2.07 (t, 2H), 1.77-1.73 (m, 2H), 1.47 (br, 2H), 1.30-1.17 (m, 14H), 0.99-0.97 (m, 1H), 0.55-0.50 (m, 6H), 0.52-0.48 (br, 1H), 0.33-0.23 (m, 2H), 0.12-0.10 (m, 1H).

### Example 98

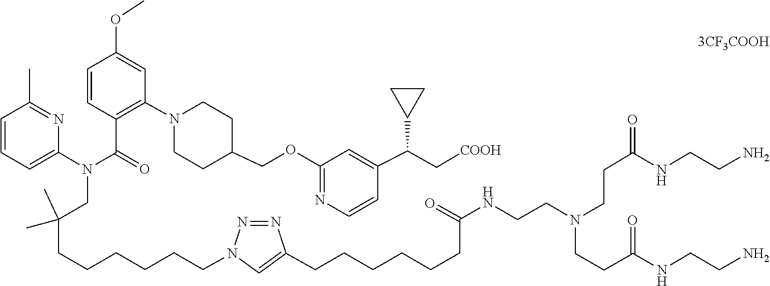 [SMILES: COc1ccc(C(=O)N(CC(C)(C)CCCCCCn2cc(CCCCCCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

MS (ESI) m/z 1151.4 [M+H]⁺. ¹H NMR (400 MHZ, DMSO-d₆): δ 8.39 (t, NH, 2H), 8.14 (t, NH, 1H), 8.04-8.03 (d, 1H), 7.86 (br, NH₃, 6H), 7.78 (s, 1H), 7.25 (br, 1H), 7.11-7.09 (d, 1H), 6.92-6.91 (d, 1H), 6.85-6.83 (d, 1H), 6.71 (s, 1H), 6.52-6.50 (d, 1H), 6.41 (br, 1H), 6.21 (s, 1H), 4.23-4.21 (t, 2H), 4.14-4.12 (d, 2H), 4.09 (br, 2H), 3.69 (s, 3H), 3.43-3.37 (m, 6H), 3.34-3.29 (m, 4H), 3.12 (br, 2H), 2.89-2.86 (m, 4H), 2.70-2.68 (d, 2H), 2.66-2.62 (m, 4H), 2.60-2.56 (m, 2H), 2.34 (s, 3H), 2.27-2.21 (m, 1H), 2.11-2.07 (t, 2H), 1.75-1.66 (m, 5H), 1.57-1.55 (m, 2H), 1.49-1.46 (m, 2H), 1.39 (br, 2H), 1.25 (br, 4H), 1.25-0.94 (m, 9H), 0.75 (s, 6H), 0.53-0.49 (m, 1H), 0.32-0.25 (m, 2H), 0.23-0.18 (m, 1H).

### Example 99

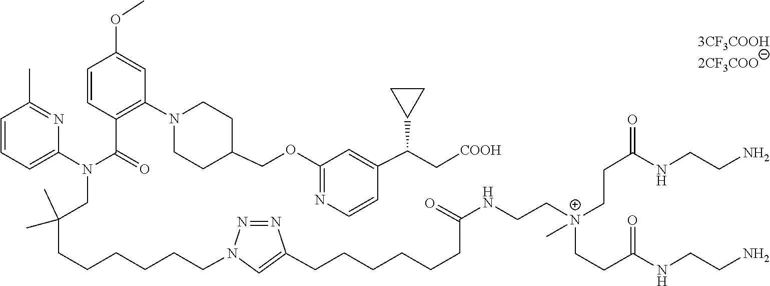 [SMILES: COc1ccc(C(=O)N(CC(C)(C)CCCCCCn2cc(CCCCCCC(=O)NCC[N+](C)(CCC(=O)NCCN)CCC(=O)NCCN)nn2)c2cccc(C)n2)c(N2CCC(COc3cc([C@@H](CC(=O)O)C4CC4)ccn3)CC2)c1]

MS (ESI) m/z 1164.5 [M]⁺. ¹H NMR (400 MHZ, DMSO-d₆): δ 8.38 (t, NH, 2H), 8.17 (t, NH, 1H), 8.05-8.03 (d, 1H), 7.89 (br, NH₃, 6H), 7.80 (s, 1H), 7.28-7.25 (br, 1H), 7.11-7.09 (d, 1H), 6.93-6.91 (d, 1H), 6.85-6.83 (d, 1H), 6.72 (s, 1H), 6.53-6.50 (d, 1H), 6.42 (br, 1H), 6.22 (s, 1H), 4.24-4.21 (t, 2H), 4.14-4.12 (d, 2H), 4.09 (br, 2H), 3.68 (s, 3H), 3.59-3.56 (m, 4H), 3.46-3.44 (m, 2H), 3.34-3.29 (m, 4H), 3.03 (s, 3H), 2.90-2.85 (m, 4H), 2.69-2.65 (m, 6H), 2.59-2.55 (t, 2H), 2.34 (s, 3H), 2.27-2.22 (m, 1H), 2.11-2.07 (t, 2H), 1.75-1.66 (m, 5H), 1.57-1.55 (m, 2H), 1.49-1.46 (m, 2H), 1.39 (br, 2H), 1.25 (br, 4H), 1.25-0.94 (m, 9H), 0.75 (s, 6H), 0.52-0.48 (m, 1H), 0.32-0.25 (m, 2H), 0.23-0.18 (m, 1H).

### Example 100

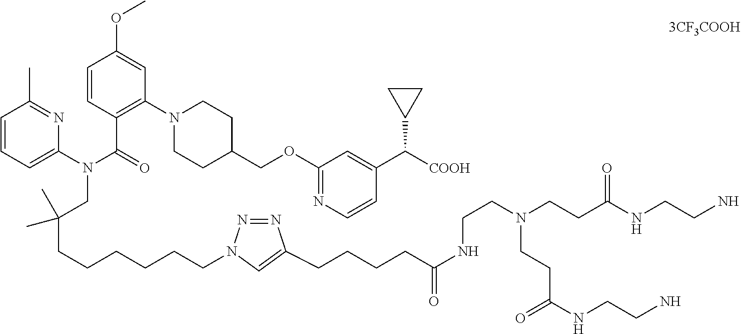 [SMILES: COc1ccc(C(=O)N(CC(C)(C)CCCCCCn2cc(CCCCC(=O)NCCN(CCC(=O)NCCN)CCC(=O)NCCN)nn2)c2cccc(C)n2)c(N2CCC(COc3cc([C@@H](C(=O)O)C4CC4)ccn3)CC2)c1]

MS (ESI) m/z 561.9 [(M+H)/2]⁺. ¹H NMR (400 MHZ, DMSO-d₆): δ 8.38 (t, NH, 2H), 8.17 (t, NH, 1H), 8.05-8.04 (d, 1H), 7.83 (br, NH₃, 6H), 7.81 (s, 1H), 7.26 (br, 1H), 7.11-7.09 (d, 1H), 6.93-6.92 (d, 1H), 6.86-6.84 (d, 1H), 6.72 (s, 1H), 6.53-6.50 (d, 1H), 6.41 (br, 1H), 6.23 (s, 1H), 4.25-4.21 (t, 2H), 4.14-4.10 (d, 4H), 3.69 (s, 3H), 3.43-3.37 (m, 6H), 3.33-3.28 (m, 4H), 3.19-3.16 (br, 2H), 2.90-2.85 (m, 2H), 2.70-2.66 (d, 6H), 2.64-2.57 (m, 6H), 2.35 (s, 3H), 2.27-2.21 (m, 1H), 2.15-2.12 (t, 2H), 1.75-1.66 (m, 5H), 1.57-1.55 (m, 2H), 1.42 (br, 2H), 1.25 (br, 4H), 1.25-0.94 (m, 9H), 0.75 (s, 6H), 0.53-0.48 (m, 1H), 0.32-0.25 (m, 2H), 0.23-0.18 (m, 1H).

### Example 101

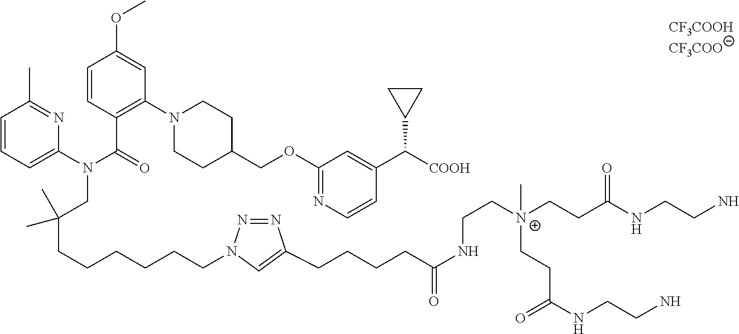 [SMILES: COc1ccc(C(=O)N(CC(C)(C)CCCCCCn2cc(CCCCC(=O)NCC[N+](C)(CCC(=O)NCCN)CCC(=O)NCCN)nn2)c2cccc(C)n2)c(N2CCC(COc3cc([C@@H](C(=O)O)C4CC4)ccn3)CC2)c1 ; "CF3COO" ; "CF3COOH"]

MS (ESI) m/z 568.9 [(M+H)/2]⁺. ¹H NMR (400 MHZ, DMSO-d₆): δ 8.35 (t, NH, 2H), 8.17 (t, NH, 1H), 8.05-8.03 (d, 1H), 7.86 (br, NH₃, 6H), 7.80 (s, 1H), 7.28-7.25 (br, 1H), 7.11-7.09 (d, 1H), 6.92-6.91 (d, 1H), 6.85-6.83 (d, 1H), 6.71 (s, 1H), 6.53-6.50 (d, 1H), 6.42 (br, 1H), 6.22 (s, 1H), 4.24-4.21 (t, 2H), 4.13-4.12 (d, 2H), 4.09 (br, 2H), 3.68 (s, 3H), 3.62-3.58 (m, 4H), 3.46-3.44 (m, 2H), 3.33-3.28 (m, 4H), 3.03 (s, 3H), 2.89-2.83 (m, 4H), 2.69-2.65 (m, 6H), 2.60-2.56 (t, 2H), 2.34 (s, 3H), 2.26-2.20 (m, 1H), 2.15-2.12 (t, 2H), 1.76-1.66 (m, 5H), 1.55-1.47 (m, 4H), 1.28-1.23 (m, 2H), 1.11-0.98 (m, 9H), 0.74 (s, 6H), 0.53-0.48 (m, 1H), 0.32-0.22 (m, 2H), 0.14-0.10 (m, 1H).

### Example 102

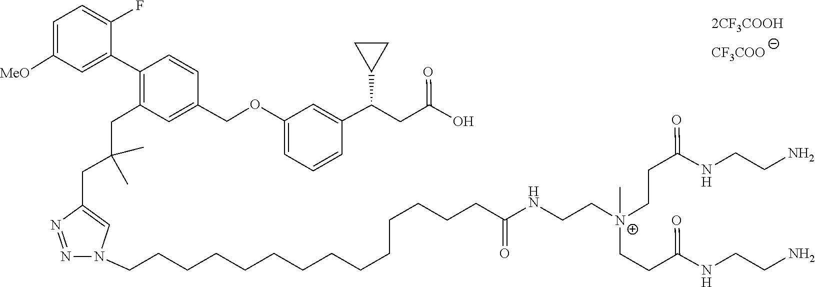 [SMILES: COc1ccc(F)c(-c2ccc(COc3cccc([C@@H](CC(=O)O)C4CC4)c3)cc2CC(C)(C)Cc2cn(CCCCCCCCCCCCCCC(=O)NCC[N+](C)(CCC(=O)NCCN)CCC(=O)NCCN)nn2)c1 ; "CF3COO"]

MS (ESI) m/z 1083.3 [M]⁺. ¹H NMR (400 MHZ, DMSO-d₆): δ 8.55 (t, NH, 2H), 8.26 (t, NH, 1H), 8.11 (br, 6H), 7.70 (s, 1H), 7.45 (s, 1H), 7.39-7.37 (d, 1H), 7.22-7.14 (m, 3H), 6.96-6.91 (m, 2H), 6.86-6.77 (m, 3H), 5.12 (s, 2H), 4.26-4.23 (t, 2H), 3.74 (s, 3H), 3.63-3.59 (m, 4H), 3.45-3.40 (m, 2H), 3.36-3.31 (m, 6H), 3.06 (s, 3H), 2.90-2.85 (m, 4H), 2.73-2.68 (m, 5H), 2.65-2.61 (m, 2H), 2.55-2.50 (m, 1H), 2.34 (s, 2H), 2.26-2.22 (m, 1H), 2.11-2.07 (t, 2H), 1.77-1.73 (m, 2H), 1.47 (br, 2H), 1.30-1.17 (m, 20H), 0.99-0.97 (m, 1H), 0.55-0.50 (m, 6H), 0.52-0.48 (br, 1H), 0.33-0.23 (m, 2H), 0.12-0.10 (m, 1H).

### Example 103

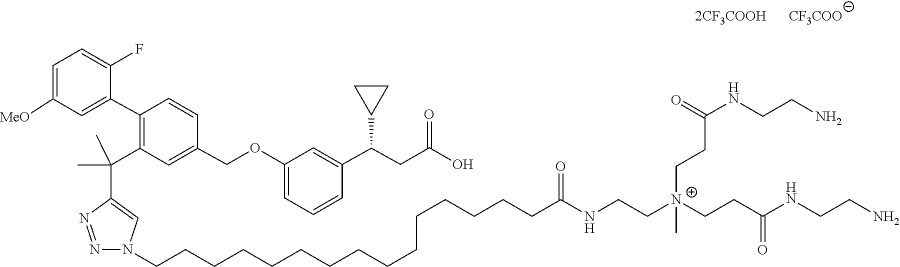 [SMILES: COc1ccc(F)c(-c2ccc(COc3cccc([C@@H](CC(=O)O)C4CC4)c3)cc2C(C)(C)c2cn(CCCCCCCCCCCCCCCC(=O)NCC[N+](C)(CCC(=O)NCCN)CCC(=O)NCCN)nn2)c1 ; "CF3COO"]

MS (ESI) m/z 1069.3 [M+H]⁺. ¹H NMR (400 MHZ, DMSO-d₆): δ 8.54-8.51 (t, NH, 2H), 8.26-8.23 (t, 1H), 8.07 (br, NH₃, 6H), 7.68 (s, 1H), 7.35-7.33 (d, 1H), 7.30 (s, 1H), 7.24-7.20 (m, 1H), 7.00-6.77 (m, 6H), 5.95-5.92 (m, 1H), 5.14 (s, 3H), 4.12-4.03 (m, 2H), 3.62-3.59 (m, 4H), 3.59 (s, 3H), 3.45-3.42 (m, 2H), 3.35-3.30 (m, 6H), 3.06 (s, 3H), 2.89-2.85 (m, 4H), 2.73-2.64 (m, 6H), 2.30-2.24 (m, 1H), 2.11-2.07 (t, 2H), 1.74-1.69 (m, 2H), 1.59 (s, 3H), 1.48-1.44 (m, 2H), 1.44 (s, 3H), 1.30-1.19 (m, 22H), 0.99-0.97 (m, 1H), 0.52-0.48 (m, 1H), 0.33-0.23 (m, 2H), 0.12-0.10 (m, 1H).

### Example 104

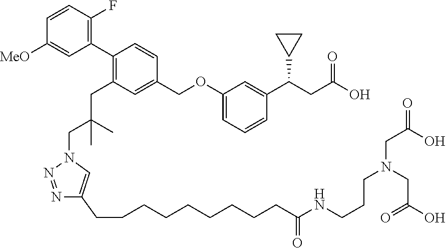 [SMILES: COc1ccc(F)c(-c2ccc(COc3cccc([C@@H](CC(=O)O)C4CC4)c3)cc2CC(C)(C)Cn2cc(CCCCCCCCCC(=O)NCCCN(CC(=O)O)CC(=O)O)nn2)c1]

MS (ESI) m/z 900.1 [M+H]⁺. ¹H NMR (400 MHZ, DMSO-d₆): δ 7.81 (br, NH, 1H), 7.62 (s, 1H), 7.44 (s, 1H), 7.41-7.39 (d, 1H), 7.23-7.12 (m, 3H), 6.95-6.91 (m, 2H), 6.86-6.82 (m, 2H), 6.79-6.77 (m, 1H), 5.13 (s, 2H), 3.97 (s, 2H), 3.81 (s, 4H), 3.73 (s, 3H), 3.09-3.03 (m, 2H), 2.97-2.94 (m, 2H), 2.73-2.50 (m, 6H), 2.27-2.21 (m, 1H), 2.05-1.99 (m, 2H), 1.66-1.63 (m, 2H), 1.56-1.53 (m, 2H), 1.48-1.45 (m, 2H), 1.27-1.23 (m, 10H), 0.99-0.97 (m, 1H), 0.52 (br, 7H), 0.33-0.23 (m, 2H), 0.12-0.10 (m, 1H).

### Example 105

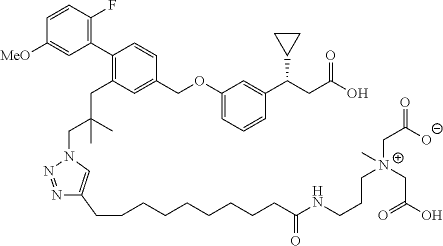 [SMILES: COc1ccc(F)c(-c2ccc(COc3cccc([C@@H](CC(=O)O)C4CC4)c3)cc2CC(C)(C)Cn2cc(CCCCCCCCCC(=O)NCCC[N+](C)(CC(=O)[O-])CC(=O)O)nn2)c1]

MS (ESI) m/z 915.1 [M+H]⁺. ¹H NMR (400 MHz, DMSO-d₆): δ 7.81 (br, NH, 1H), 7.62 (s, 1H), 7.44 (s, 1H), 7.41-7.39 (d, 1H), 7.23-7.12 (m, 3H), 6.95-6.91 (m, 2H), 6.86-6.82 (m, 2H), 6.79-6.77 (m, 1H), 5.14 (s, 2H), 4.34 (s, 4H), 3.98 (s, 2H), 3.73 (s, 3H), 3.65-3.61 (m, 2H, 3.27 (s, 3H), 3.09-3.05 (m, 2H), 2.73-2.50 (m, 6H), 2.28-2.22 (m, 1H), 2.05-1.99 (m, 2H), 1.83-1.79 (m, 2H), 1.56-1.53 (m, 2H), 1.49-1.46 (m, 2H), 1.27-1.23 (m, 10H), 0.99-0.97 (m, 1H), 0.52 (br, 7H), 0.33-0.23 (m, 2H), 0.12-0.10 (m, 1H).

### Example 106

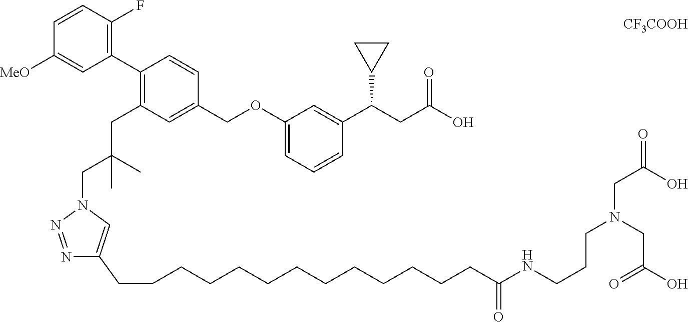 [SMILES: COc1ccc(F)c(-c2ccc(COc3cccc([C@@H](CC(=O)O)C4CC4)c3)cc2CC(C)(C)Cn2cc(CCCCCCCCCCCCCC(=O)NCCCN(CC(=O)O)CC(=O)O)nn2)c1 ; "CF3COOH"]

MS (ESI) m/z 478.7 [(M+H)/2]⁺. ¹H NMR (400 MHZ, DMSO-d₆): δ 7.79 (br, NH, 1H), 7.62 (s, 1H), 7.44 (s, 1H), 7.41-7.39 (d, 1H), 7.23-7.12 (m, 3H), 6.95-6.91 (m, 2H), 6.86-6.82 (m, 2H), 6.79-6.77 (m, 1H), 5.14 (s, 2H), 3.98 (s, 2H), 3.73 (s, 3H), 3.70 (s, 4H), 3.08-3.04 (m, 2H), 2.90-2.85 (m, 2H), 2.73-2.51 (m, 6H), 2.28-2.22 (m, 1H), 2.04-1.99 (m, 2H), 1.62-1.54 (m, 4H), 1.48-1.45 (m, 2H), 1.27-1.23 (m, 18H), 0.99-0.97 (m, 1H), 0.52 (br, 7H), 0.33-0.23 (m, 2H), 0.12-0.10 (m, 1H).

### Example 107

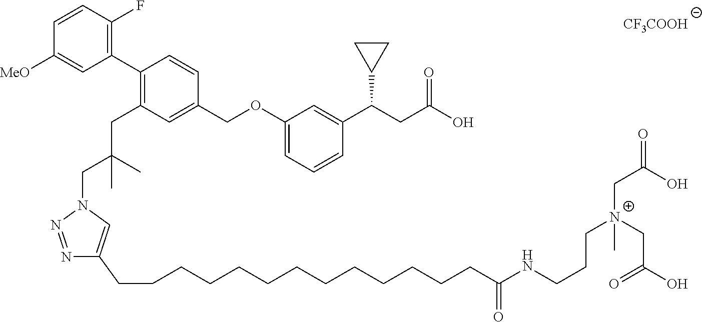 [SMILES: COc1ccc(F)c(-c2ccc(COc3cccc([C@@H](CC(=O)O)C4CC4)c3)cc2CC(C)(C)Cn2cc(CCCCCCCCCCCCCC(=O)NCCC[N+](C)(CC(=O)O)CC(=O)O)nn2)c1 ; "CF3COOH"]

MS (ESI) m/z 485.8 [(M+H)/2]⁺. ¹H NMR (400 MHZ, DMSO-d₆): δ 7.81 (br, NH, 1H), 7.62 (s, 1H), 7.44 (s, 1H), 7.41-7.39 (d, 1H), 7.23-7.12 (m, 3H), 6.95-6.91 (m, 2H), 6.86-6.82 (m, 2H), 6.79-6.77 (m, 1H), 5.14 (s, 2H), 4.46 (s, 4H), 3.98 (s, 2H), 3.73 (s, 3H), 3.67-3.63 (m, 2H), 3.31 (s, 3H), 3.11-3.06 (m, 2H), 2.73-2.50 (m, 6H), 2.28-2.22 (m, 1H), 2.06-2.02 (m, 2H), 1.83-1.80 (m, 2H), 1.56-1.47 (m, 4H), 1.27-1.23 (m, 18H), 0.99-0.97 (m, 1H), 0.52 (br, 7H), 0.33-0.23 (m, 2H), 0.12-0.10 (m, 1H).

### Example 108

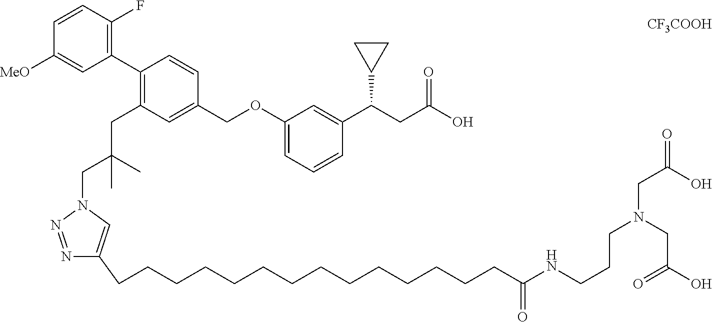 [SMILES: COc1ccc(F)c(-c2ccc(COc3cccc([C@@H](CC(=O)O)C4CC4)c3)cc2CC(C)(C)Cn2cc(CCCCCCCCCCCCCCC(=O)NCCCN(CC(=O)O)CC(=O)O)nn2)c1 ; "CF3COOH"]

MS (ESI) m/z 971.2 [M+H]⁺. ¹H NMR (400 MHZ, DMSO-d₆): δ 7.77-75 (br, NH, 1H), 7.62 (s, 1H), 7.44 (s, 1H), 7.41-7.39 (d, 1H), 7.23-7.12 (m, 3H), 6.95-6.91 (m, 2H), 6.86-6.82 (m, 2H), 6.79-6.77 (m, 1H), 5.14 (s, 2H), 3.98 (s, 2H), 3.73 (s, 3H), 3.48 (s, 4H), 3.08-3.04 (m, 2H), 2.73-2.51 (m, 8H), 2.28-2.22 (m, 1H), 2.04-2.00 (m, 2H), 1.54-1.46 (m, 6H), 1.27-1.23 (m, 20H), 0.99-0.97 (m, 1H), 0.52 (br, 7H), 0.33-0.23 (m, 2H), 0.12-0.10 (m, 1H)

### Example 109

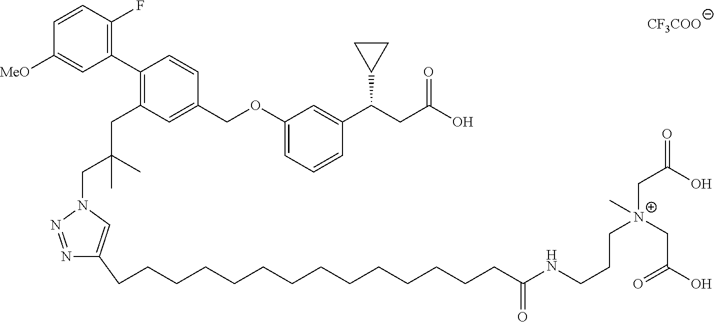 [SMILES: COc1ccc(F)c(-c2ccc(COc3cccc([C@@H](CC(=O)O)C4CC4)c3)cc2CC(C)(C)Cn2cc(CCCCCCCCCCCCCCC(=O)NCCC[N+](C)(CC(=O)O)CC(=O)O)nn2)c1 ; "CF3COO"]

MS (ESI) m/z 492.8 [(M+H)/2]⁺. ¹H NMR (400 MHZ, DMSO-d₆): δ 7.91-7.88 (br, NH, 1H), 7.62 (s, 1H), 7.44 (s, 1H), 7.41-7.39 (d, 1H), 7.23-7.12 (m, 3H), 6.95-6.91 (m, 2H), 6.86-6.82 (m, 2H), 6.79-6.77 (m, 1H), 5.14 (s, 2H), 4.42 (s, 4H), 3.98 (s, 2H), 3.73 (s, 3H), 3.66-3.62 (m, 2H), 3.30 (s, 3H), 3.09-3.07 (m, 2H), 2.73-2.50 (m, 6H), 2.28-2.22 (m, 1H), 2.06-2.02 (m, 2H), 1.84-1.81 (m, 2H), 1.56-1.47 (m, 4H), 1.27-1.23 (m, 20H), 0.99-0.97 (m, 1H), 0.52 (br, 7H), 0.33-0.23 (m, 2H), 0.12-0.10 (m, 1H).

### Example 110

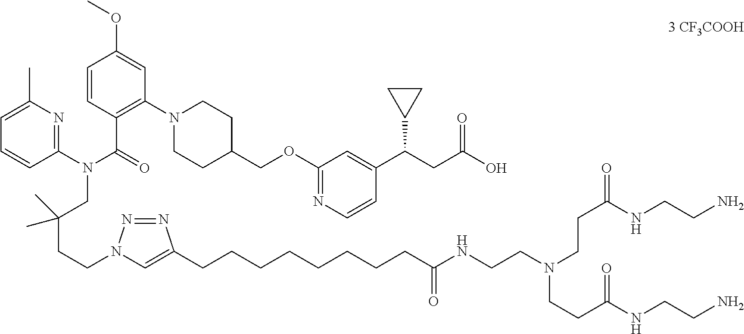 [SMILES: COc1ccc(C(=O)N(CC(C)(C)CCn2cc(CCCCCCCCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

MS (ESI) m/z 561.8 [M+H]⁺. ¹H NMR (400 MHZ, DMSO-d₆): δ 8.41 (t, NH, 2H), 8.15 (t, NH, 1H), 8.06-8.04 (d, 1H), 7.89 br, NH₃, 6H), 7.70 (s, 1H), 7.27 (br, 1H), 7.17-7.15 (d, 1H), 6.93-6.92 (d, 1H), 6.86-6.84 (d, 1H), 6.73 (s, 1H), 6.55-6.52 (d, 1H), 6.46 (br, 1H), 6.22 (s, 1H), 4.37-4.32 (t, 2H), 4.20 (br, 2H), 4.14-4.12 (d, 2H), 3.69 (s, 3H), 3.43-3.37 (m, 6H), 3.34-3.30 (m, 4H), 3.21-3.18 (br, 2H), 2.89-2.86 (m, 4H), 2.79-2.77 (d, 2H), 2.72-2.68 (m, 4H), 2.65-2.62 (m, 2H), 2.37 (s, 3H), 2.27-2.21 (m, 1H), 2.11-2.07 (t, 2H), 1.77-1.66 (m, 5H), 1.58-1.47 (m, 6H), 1.25 (br, 8H), 1.03-0.96 (m, 1H), 0.75 (s, 6H), 0.54-0.50 (m, 1H), 0.32-0.25 (m, 2H), 0.23-0.18 (m, 1H).

### Example 111

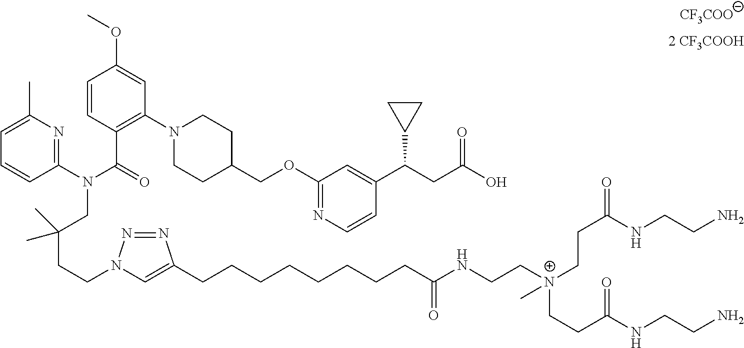 [SMILES: COc1ccc(C(=O)N(CC(C)(C)CCn2cc(CCCCCCCCC(=O)NCC[N+](C)(CCC(=O)NCCN)CCC(=O)NCCN)nn2)c2cccc(C)n2)c(N2CCC(COc3cc([C@@H](CC(=O)O)C4CC4)ccn3)CC2)c1 ; "CF3COO"]

MS (ESI) m/z 1137.3 [M+H]⁺. ¹H NMR (400 MHZ, DMSO-d₆): δ 8.42 (t, NH, 2H), 8.18 (t, NH, 1H), 8.06-8.04 (d, 1H), 7.96 (br, NH₃, 6H), 7.69 (s, 1H), 7.30-7.26 (br, 1H), 7.16-7.14 (d, 1H), 6.92-6.91 (d, 1H), 6.86-6.84 (d, 1H), 6.72 (s, 1H), 6.54-6.51 (d, 1H), 6.46-6.45 (br, 1H), 6.21 (s, 1H), 4.32-4.30 (t, 2H), 4.13-4.11 (d, 2H), 4.19 (br, 2H), 3.69 (s, 3H), 3.61-3.57 (m, 4H), 3.46-3.44 (m, 2H), 3.34-3.29 (m, 6H), 3.04 (s, 3H), 2.90-2.85 (m, 4H), 2.71−−2.67 (m, 6H), 2.58-2.54 (t, 2H), 2.36 (s, 3H), 2.26-2.20 (m, 1H), 2.11-2.07 (t, 2H), 1.75-1.66 (m, 5H), 1.57-1.46 (m, 6H), 1.25 (br, 8H), 1.04-0.94 (m, 1H), 0.75 (s, 6H), 0.54-0.50 (m, 1H), 0.32-0.25 (m, 2H), 0.23-0.18 (m, 1H).

### Example 112

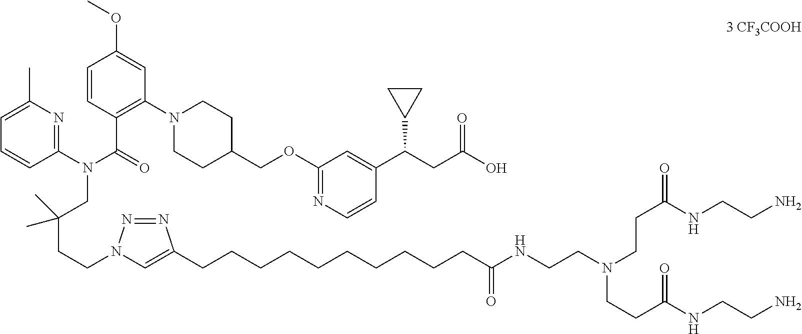 [SMILES: COc1ccc(C(=O)N(CC(C)(C)CCn2cc(CCCCCCCCCCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

MS (ESI) m/z 575.9 [(M+2H)/2]⁺. ¹H NMR (400 MHZ, DMSO-d₆): δ 8.43 (t, NH, 2H), 8.15 (t, NH, 1H), 8.05-8.04 (d, 1H), 7.94 br, NH₃, 6H), 7.69 (s, 1H), 7.28 (br, 1H), 7.17-6.14 (d, 1H), 6.93-6.91 (d, 1H), 6.86-6.84 (d, 1H), 6.72 (s, 1H), 6.53-6.51 (d, 1H), 6.46 (br, 1H), 6.21 (s, 1H), 4.36-4.32 (t, 2H), 4.18 (br, 2H), 4.13-4.12 (d, 2H), 3.69 (s, 3H), 3.43-3.36 (m, 6H), 3.33-3.29 (m, 4H), 3.20-3.17 (br, 2H), 2.90-2.86 (m, 4H), 2.69-2.63 (d, 6H), 2.58-2.54 (m, 2H), 2.35 (s, 3H), 2.27-2.21 (m, 1H), 2.10-2.07 (t, 2H), 1.76-1.65 (m, 5H), 1.55-1.47 (m, 6H), 1.25 (br, 8H), 1.00-0.98 (m, 1H), 0.78 (s, 6H), 0.52-0.50 (m, 1H), 0.32-0.25 (m, 2H), 0.23-0.18 (m, 1H).

### Example 113

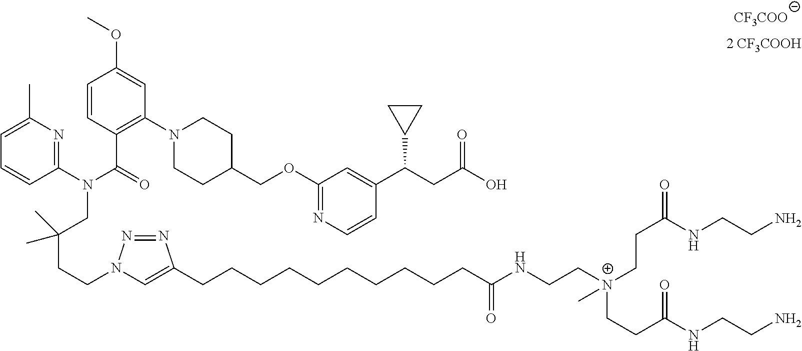 [SMILES: COc1ccc(C(=O)N(CC(C)(C)CCn2cc(CCCCCCCCCCC(=O)NCC[N+](C)(CCC(=O)NCCN)CCC(=O)NCCN)nn2)c2cccc(C)n2)c(N2CCC(COc3cc([C@@H](CC(=O)O)C4CC4)ccn3)CC2)c1 ; "CF3COO"]

MS (ESI) m/z 582.8 [(M+H)/2]⁺. ¹H NMR (400 MHZ, DMSO-d₆): δ 8.45 (t, NH, 2H), 8.20 (t, NH, 1H), 8.05-8.04 (d, 1H), 7.98 (br, NH₃, 6H), 7.69 (s, 1H), 7.30-7.26 (br, 1H), 7.16-7.14 (d, 1H), 6.93-6.92 (d, 1H), 6.86-6.84 (d, 1H), 6.72 (s, 1H), 6.54-6.51 (d, 1H), 6.47-6.46 (br, 1H), 6.22 (s, 1H), 4.35-4.31 (t, 2H), 4.18 (br, 2H), 4.13-4.12 (d, 2H), 3.69 (s, 3H), 3.61-3.57 (m, 4H), 3.47-3.45 (m, 2H), 3.34-3.30 (m, 6H), 3.05 (s, 3H), 2.90-2.86 (m, 4H), 2.71-2.67 (m, 6H), 2.58-2.54 (t, 2H), 2.36 (s, 3H), 2.26-2.22 (m, 1H), 2.11-2.07 (t, 2H), 1.76-1.65 (m, 5H), 1.57-1.46 (m, 6H), 1.25 (br, 8H), 1.04-0.94 (m, 1H), 0.75 (s, 6H), 0.54-0.50 (m, 1H), 0.32-0.25 (m, 2H), 0.23-0.18 (m, 1H).

### Example 114

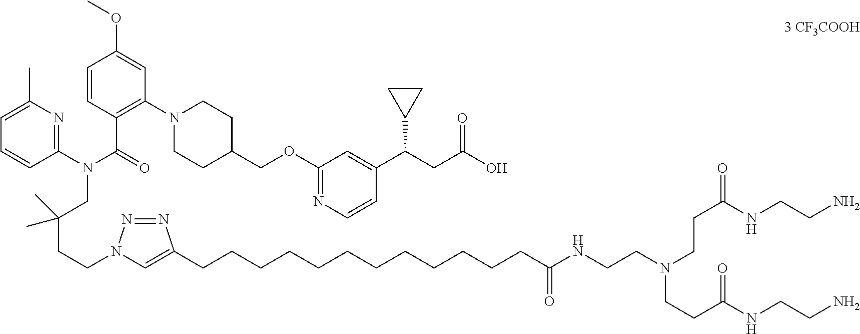 [SMILES: COc1ccc(C(=O)N(CC(C)(C)CCn2cc(CCCCCCCCCCCCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

MS (ESI) m/z 589.8 [(M+2H)/2]⁺. ¹H NMR (400 MHZ, DMSO-d₆): δ 8.40 (t, NH, 2H), 8.15 (t, NH, 1H), 8.06-8.05 (d, 1H), 7.87 br, NH₃, 6H), 7.70 (s, 1H), 7.31-7.27 (br, 1H), 7.17-6.15 (d, 1H), 6.94-6.93 (d, 1H), 6.87-6.85 (d, 1H), 6.73 (s, 1H), 6.54-6.53 (d, 1H), 6.47 (br, 1H), 6.23 (s, 1H), 4.34-4.30 (t, 2H), 4.20 (br, 2H), 4.14-4.12 (d, 2H), 3.70 (s, 3H), 3.43-3.37 (m, 6H), 3.34-3.30 (m, 4H), 3.20-3.17 (br, 2H), 2.91-2.87 (m, 4H), 2.70-2.68 (d, 2H), 2.65-2.62 (t, 2H), 2.59-2.57 (m, 2H), 2.37 (s, 3H), 2.28-2.23 (m, 1H), 2.11-2.08 (t, 2H), 1.76-1.66 (m, 5H), 1.58-1.47 (m, 6H), 1.25 (br, 16H), 1.02-0.99 (m, 1H), 0.80 (s, 6H), 0.52-0.50 (m, 1H), 0.32-0.25 (m, 2H), 0.23-0.18 (m, 1H).

### Example 115

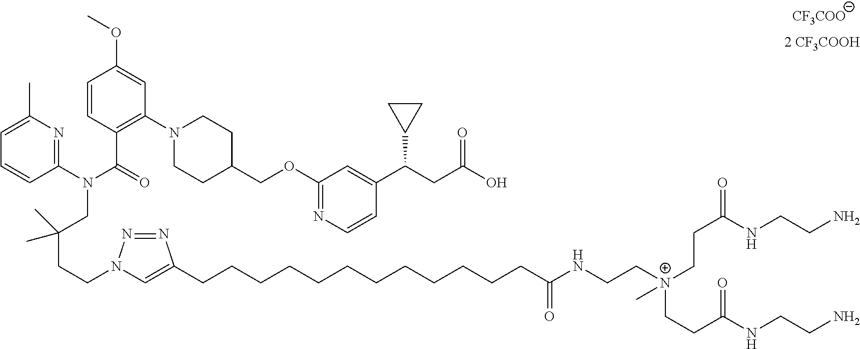 [SMILES: COc1ccc(C(=O)N(CC(C)(C)CCn2cc(CCCCCCCCCCCCC(=O)NCC[N+](C)(CCC(=O)NCCN)CCC(=O)NCCN)nn2)c2cccc(C)n2)c(N2CCC(COc3cc([C@@H](CC(=O)O)C4CC4)ccn3)CC2)c1 ; "CF3COO"]

MS (ESI) m/z 596.9 [(M+H)/2]⁺. ¹H NMR (400 MHZ, DMSO-d₆): δ 8.53 (t, NH, 2H), 8.25 (t, NH, 1H), 8.08 (br, NH₃, 6H), 8.05-8.04 (d, 1H), 7.69 (s, 1H), 7.30-7.26 (br, 1H), 7.16-7.14 (d, 1H), 6.93-6.92 (d, 1H), 6.86-6.84 (d, 1H), 6.73 (s, 1H), 6.54-6.51 (d, 1H), 6.47-6.46 (br, 1H), 6.22 (s, 1H), 4.34-4.31 (t, 2H), 4.18 (br, 2H), 4.13-4.12 (d, 2H), 3.70 (s, 3H), 3.63-3.59 (m, 4H), 3.47-3.44 (m, 2H), 3.35-3.31 (m, 6H), 3.06 (s, 3H), 2.90-2.86 (m, 4H), 2.73-2.68 (m, 6H), 2.58-2.54 (t, 2H), 2.35 (s, 3H), 2.27-2.22 (m, 1H), 2.11-2.07 (t, 2H), 1.76-1.65 (m, 5H), 1.56-1.46 (m, 6H), 1.25 (br, 16H), 1.04-0.94 (m, 1H), 0.80 (s, 6H), 0.54-0.50 (m, 1H), 0.32-0.25 (m, 2H), 0.23-0.18 (m, 1H).

### Example 116

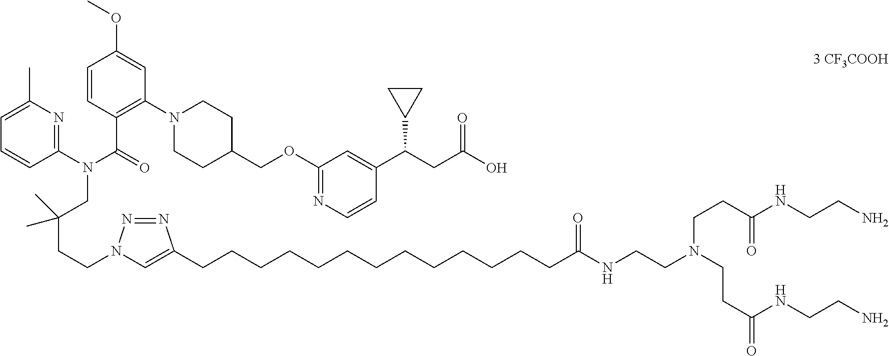 [SMILES: COc1ccc(C(=O)N(CC(C)(C)CCn2cc(CCCCCCCCCCCCCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

MS (ESI) m/z 596.8 [(M+2H)/2]⁺. ¹H NMR (400 MHZ, DMSO-d₆): δ 8.41 (t, NH, 2H), 8.14 (t, NH, 1H), 8.05-8.04 (d, 1H), 7.89 br, NH₃, 6H), 7.69 (s, 1H), 7.31-7.27 (br, 1H), 7.16-6.14 (d, 1H), 6.93-6.92 (d, 1H), 6.86-6.84 (d, 1H), 6.72 (s, 1H), 6.54-6.52 (d, 1H), 6.47 (br, 1H), 6.22 (s, 1H), 4.35-4.33 (t, 2H), 4.19 (br, 2H), 4.14-4.12 (d, 2H), 3.69 (s, 3H), 3.42-3.37 (m, 6H), 3.34-3.29 (m, 4H), 3.19-3.16 (br, 2H), 2.91-2.86 (m, 4H), 2.70-2.68 (d, 2H), 2.65-2.61 (t, 2H), 2.59-2.55 (m, 2H), 2.36 (s, 3H), 2.27-2.21 (m, 1H), 2.11-2.08 (t, 2H), 1.76-1.66 (m, 5H), 1.58-1.47 (m, 6H), 1.25 (br, 18H), 1.02-0.99 (m, 1H), 0.80 (s, 6H), 0.52-0.50 (m, 1H), 0.32-0.25 (m, 2H), 0.23-0.18 (m, 1H).

### Example 117

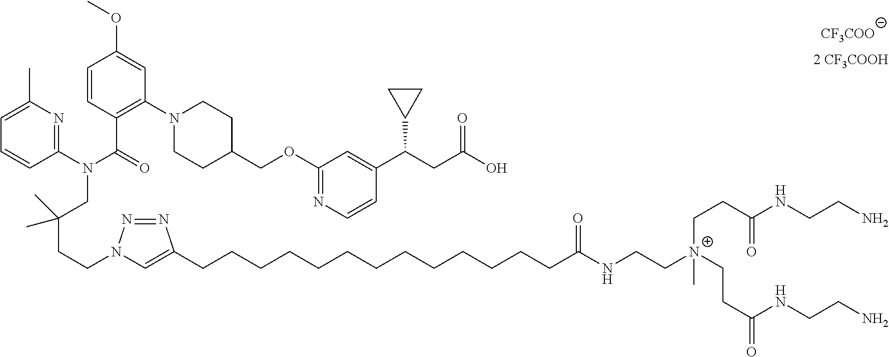 [SMILES: COc1ccc(C(=O)N(CC(C)(C)CCn2cc(CCCCCCCCCCCCCC(=O)NCC[N+](C)(CCC(=O)NCCN)CCC(=O)NCCN)nn2)c2cccc(C)n2)c(N2CCC(COc3cc([C@@H](CC(=O)O)C4CC4)ccn3)CC2)c1 ; "CF3COO"]

MS (ESI) m/z 603.9 [(M+H)/2]⁺. ¹H NMR (400 MHZ, DMSO-d₆): δ 8.46 (t, NH, 2H), 8.20 (t, NH, 1H), 8.05-8.04 (d, 1H), 8.00 (br, NH₃, 6H), 7.69 (s, 1H), 7.30-7.26 (br, 1H), 7.18-7.16 (d, 1H), 6.93-6.92 (d, 1H), 6.86-6.85 (d, 1H), 6.73 (s, 1H), 6.54-6.51 (d, 1H), 6.47-6.46 (br, 1H), 6.22 (s, 1H), 4.33-4.31 (t, 2H), 4.19 (br, 2H), 4.14-4.12 (d, 2H), 3.71 (s, 3H), 3.62-3.58 (m, 4H), 3.47-3.45 (m, 2H), 3.35-3.30 (m, 6H), 3.05 (s, 3H), 2.90-2.86 (m, 4H), 2.72-2.68 (m, 6H), 2.58-2.55 (m, 2H), 2.36 (s, 3H), 2.27-2.21 (m, 1H), 2.11-2.07 (t, 2H), 1.76-1.66 (m, 5H), 1.57-1.47 (m, 6H), 1.25 (br, 18H), 1.02-0.98 (m, 1H), 0.80 (s, 6H), 0.53-0.50 (m, 1H), 0.32-0.25 (m, 2H), 0.23-0.18 (m, 1H).

### Example 118

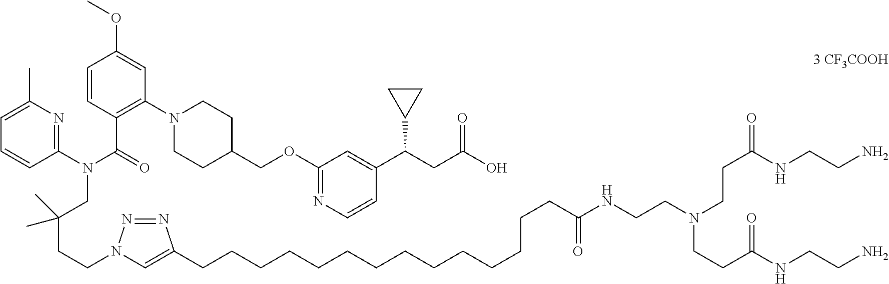 [SMILES: COc1ccc(C(=O)N(CC(C)(C)CCn2cc(CCCCCCCCCCCCCCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

MS (ESI) m/z 603.9 [(M+2H)/2]⁺. ¹H NMR (400 MHZ, DMSO-d₆): δ 8.40 (t, NH, 2H), 8.14 (t, NH, 1H), 8.05-8.04 (d, 1H), 7.87 br, NH₃, 6H), 7.69 (s, 1H), 7.30-7.26 (br, 1H), 7.16-6.14 (d, 1H), 6.93-6.92 (d, 1H), 6.86-6.84 (d, 1H), 6.72 (s, 1H), 6.53-6.52 (d, 1H), 6.47 (br, 1H), 6.22 (s, 1H), 4.35-4.33 (t, 2H), 4.19 (br, 2H), 4.14-4.12 (d, 2H), 3.69 (s, 3H), 3.42-3.37 (m, 6H), 3.33-3.29 (m, 4H), 3.19-3.17 (br, 2H), 2.90-2.86 (m, 4H), 2.70-2.68 (d, 2H), 2.65-2.61 (t, 2H), 2.59-2.55 (m, 2H), 2.36 (s, 3H), 2.27-2.21 (m, 1H), 2.11-2.07 (t, 2H), 1.76-1.66 (m, 5H), 1.58-1.46 (m, 6H), 1.25 (br, 20H), 1.02-0.98 (m, 1H), 0.80 (s, 6H), 0.52-0.50 (m, 1H), 0.32-0.25 (m, 2H), 0.23-0.18 (m, 1H).

### Example 119

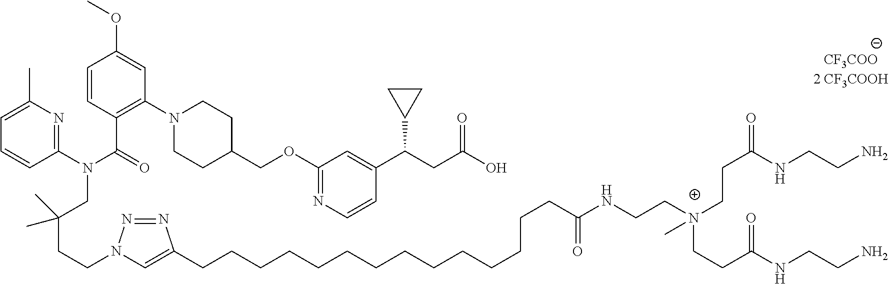

MS (ESI) m/z 610.9 [(M+H)/2]⁺. ¹H NMR (400 MHZ, DMSO-d₆): δ 8.54 (t, NH, 2H), 8.25 (t, NH, 1H), 8.08 (br, NH₃, 6H), 8.05-8.04 (d, 1H), 7.69 (s, 1H), 7.30-7.26 (br, 1H), 7.16-7.14 (d, 1H), 6.93-6.92 (d, 1H), 6.86-6.84 (d, 1H), 6.73 (s, 1H), 6.53-6.51 (d, 1H), 6.47-6.46 (br, 1H), 6.22 (s, 1H), 4.33-4.31 (t, 2H), 4.18 (br, 2H), 4.13-4.12 (d, 2H), 3.69 (s, 3H), 3.63-3.59 (m, 4H), 3.47-3.45 (m, 2H), 3.34-3.31 (m, 6H), 3.06 (s, 3H), 2.90-2.86 (m, 4H), 2.73-2.67 (m, 6H), 2.58-2.54 (m, 2H), 2.35 (s, 3H), 2.27-2.21 (m, 1H), 2.11-2.07 (t, 2H), 1.76-1.65 (m, 5H), 1.55-1.47 (m, 6H), 1.25 (br, 20H), 1.02-0.98 (m, 1H), 0.80 (s, 6H), 0.52-0.50 (m, 1H), 0.32-0.25 (m, 2H), 0.23-0.18 (m, 1H).

### Example 120

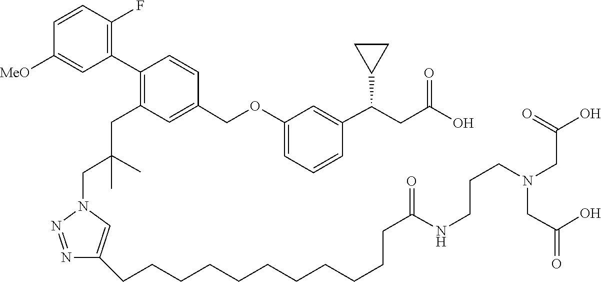 [SMILES: COc1ccc(F)c(-c2ccc(COc3cccc([C@@H](CC(=O)O)C4CC4)c3)cc2CC(C)(C)Cn2cc(CCCCCCCCCCCC(=O)NCCCN(CC(=O)O)CC(=O)O)nn2)c1]

MS (ESI) m/z 928.2 [M+H]⁺. ¹H NMR (400 MHZ, DMSO-d₆): δ 7.81 (br, NH, 1H), 7.63 (s, 1H), 7.44 (s, 1H), 7.41-7.39 (d, 1H), 7.23-7.11 (m, 3H), 6.95-6.91 (m, 2H), 6.86-6.82 (m, 2H), 6.79-6.77 (m, 1H), 5.14 (s, 2H), 3.98 (s, 2H), 3.85 (s, 4H), 3.74 (s, 3H), 3.08-3.04 (m, 2H), 3.02-2.97 (m, 2H), 2.73-2.51 (m, 6H), 2.28-2.22 (m, 1H), 2.05-2.01 (t, 2H), 1.68-1.65 (m, 2H), 1.54-1.46 (m, 4H), 1.27-1.23 (m, 14H), 0.99-0.97 (m, 1H), 0.52 (br, 7H), 0.33-0.23 (m, 2H), 0.12-0.10 (m, 1H).

### Example 121

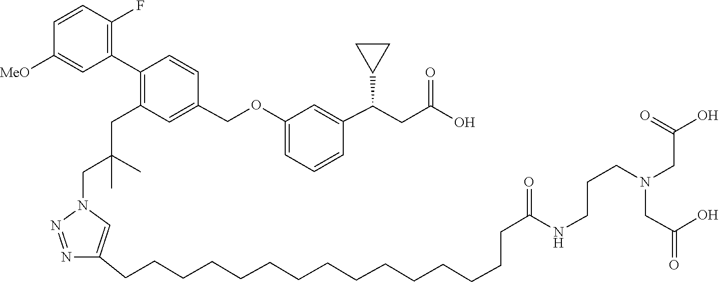 [SMILES: COc1ccc(F)c(-c2ccc(COc3cccc([C@@H](CC(=O)O)C4CC4)c3)cc2CC(C)(C)Cn2cc(CCCCCCCCCCCCCCCC(=O)NCCCN(CC(=O)O)CC(=O)O)nn2)c1]

MS (ESI) m/z 984.3 [M+H]⁺. ¹H NMR (400 MHZ, DMSO-d₆): δ 7.81 (br, NH, 1H), 7.63 (s, 1H), 7.44 (s, 1H), 7.41-7.39 (d, 1H), 7.25-7.12 (m, 3H), 6.95-6.91 (m, 2H), 6.86-6.82 (m, 2H), 6.79-6.77 (m, 1H), 5.14 (s, 2H), 3.98 (s, 2H), 3.85 (s, 4H), 3.73 (s, 3H), 3.08-3.04 (m, 2H), 3.01-2.96 (m, 2H), 2.73-2.51 (m, 6H), 2.28-2.22 (m, 1H), 2.05-2.01 (t, 2H), 1.67-1.64 (m, 2H), 1.54-1.46 (m, 4H), 1.27-1.23 (m, 22H), 1.00-0.97 (m, 1H), 0.52 (br, 7H), 0.33-0.23 (m, 2H), 0.12-0.10 (m, 1H).

### Example 122

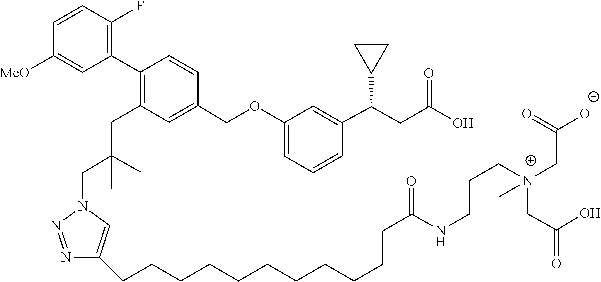 [SMILES: COc1ccc(F)c(-c2ccc(COc3cccc([C@@H](CC(=O)O)C4CC4)c3)cc2CC(C)(C)Cn2cc(CCCCCCCCCCCC(=O)NCCC[N+](C)(CC(=O)[O-])CC(=O)O)nn2)c1]

MS (ESI) m/z 942.3 [M]⁺. ¹H NMR (400 MHZ, DMSO-d₆): δ 7.81 (br, NH, 1H), 7.62 (s, 1H), 7.44 (s, 1H), 7.42-7.40 (d, 1H), 7.23-7.12 (m, 3H), 6.95-6.91 (m, 2H), 6.86-6.82 (m, 2H), 6.79-6.77 (m, 1H), 5.14 (s, 2H), 4.33 (s, 4H), 3.98 (s, 2H), 3.74 (s, 3H), 3.65-3.61 (m, 2H), 3.27 (s, 3H), 3.09-3.05 (m, 2H), 2.73-2.50 (m, 6H), 2.28-2.22 (m, 1H), 2.05-2.01 (m, 2H), 1.83-1.79 (m, 2H), 1.56-1.47 (m, 4H), 1.27-1.23 (m, 14H), 0.99-0.97 (m, 1H), 0.52 (br, 7H), 0.33-0.23 (m, 2H), 0.12-0.10 (m, 1H).

### Example 123

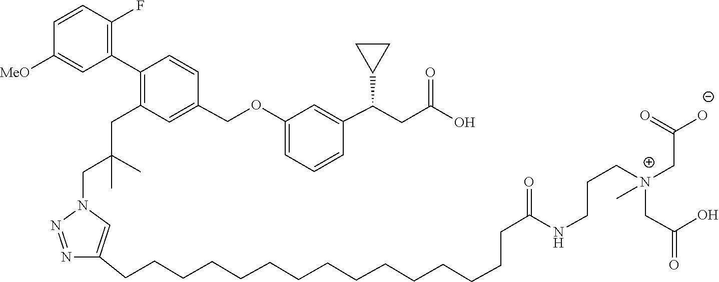 [SMILES: COc1ccc(F)c(-c2ccc(COc3cccc([C@@H](CC(=O)O)C4CC4)c3)cc2CC(C)(C)Cn2cc(CCCCCCCCCCCCCCCC(=O)NCCC[N+](C)(CC(=O)[O-])CC(=O)O)nn2)c1]

MS (ESI) m/z 998.3 [M]⁺. ¹H NMR (400 MHZ, DMSO-d₆): δ 7.87 (br, NH, 1H), 7.62 (s, 1H), 7.44 (s, 1H), 7.42-7.40 (d, 1H), 7.23-7.12 (m, 3H), 6.95-6.91 (m, 2H), 6.86-6.82 (m, 2H), 6.79-6.77 (m, 1H), 5.13 (s, 2H), 4.29 (s, 4H), 3.97 (s, 2H), 3.73 (s, 3H), 3.64-3.61 (m, 2H), 3.25 (s, 3H), 3.08-3.04 (m, 2H), 2.73-2.50 (m, 6H), 2.27-2.21 (m, 1H), 2.05-2.01 (m, 2H), 1.83-1.79 (m, 2H), 1.56-1.47 (m, 4H), 1.27-1.23 (m, 22H), 0.99-0.97 (m, 1H), 0.52 (br, 7H), 0.33-0.23 (m, 2H), 0.12-0.10 (m, 1H).

### Example 124

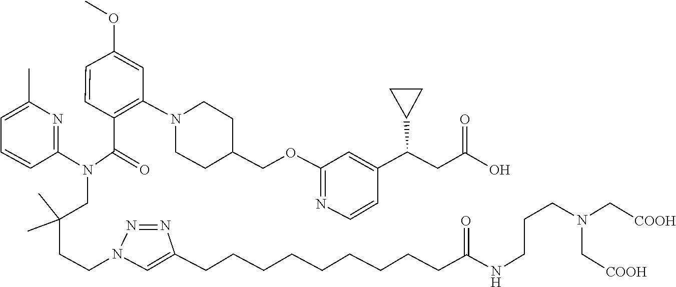 [SMILES: COc1ccc(C(=O)N(CC(C)(C)CCn2cc(CCCCCCCCCC(=O)NCCCN(CC(=O)O)CC(=O)O)nn2)c2cccc(C)n2)c(N2CCC(COc3cc([C@@H](CC(=O)O)C4CC4)ccn3)CC2)c1]

MS (ESI) m/z 1038.3 [M+H]⁺. ¹H NMR (400 MHZ, DMSO-d₆): δ 8.06-8.05 (d, 1H), 7.86 (t, NH, 1H), 7.69 (s, 1H), 7.30-7.26 (br, 1H), 7.17-7.15 (d, 1H), 6.94-6.93 (d, 1H), 6.87-6.85 (d, 1H), 6.74 (s, 1H), 6.54-6.52 (d, 1H), 6.48-6.46 (br, 1H), 6.22 (s, 1H), 4.35-4.31 (t, 2H), 4.19 (br, 2H), 4.14-4.12 (d, 2H), 4.04 (s, 4H), 3.69 (s, 3H), 3.20-3.15 (m, B, 2H), 3.09-3.05 (m, 2H), 2.70-2.68 (m, 2H), 2.59-2.55 (m, 2H), 2.36 (s, 3H), 2.26-2.22 (m, 1H), 2.06-2.02 (t, 2H), 1.78-1.74 (m, 3H), 1.68-1.65 (m, 4H), 1.58-1.55 (m, 2H), 1.46-1.43 (m, 2H), 1.36-1.20 (br, 12H), 1.02-0.98 (m, 1H), 0.78 (s, 6H), 0.52-0.50 (m, 1H), 0.32-0.25 (m, 2H), 0.23-0.18 (m, 1H).

### Example 125

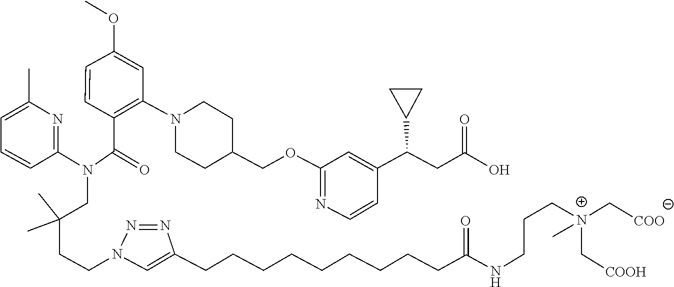

MS (ESI) m/z 526.8 [(M+H)/2]⁺. ¹H NMR (400 MHz, DMSO-d₆): δ 8.05-8.04 (d, 1H), 7.89 (t, NH, 1H), 7.68 (s, 1H), 7.30-7.26 (br, 1H), 7.16-7.14 (d, 1H), 6.93-6.91 (d, 1H), 6.86-6.84 (d, 1H), 6.72 (s, 1H), 6.53-6.51 (d, 1H), 6.48-6.46 (br, 1H), 6.21 (s, 1H), 4.46 (s, 4H), 4.33 (t, 2H), 4.18 (br, 2H), 4.13-4.12 (d, 2H), 3.69 (s, 3H), 3.67-3.63 (m, B, 2H), 3.31 (s, 3H), 3.09-3.07 (m, 2H), 2.69-2.67 (m, 2H), 2.58-2.54 (m, 2H), 2.36 (s, 3H), 2.27-2.21 (m, 1H), 2.05-2.02 (t, 2H), 1.85-1.73 (m, 3H), 1.70-1.64 (m, 4H), 1.57-1.53 (m, 2H), 1.50-1.45 (m, 2H), 1.36-1.20 (br, 12H), 1.02-0.98 (m, 1H), 0.78 (s, 6H), 0.52-0.50 (m, 1H), 0.32-0.25 (m, 2H), 0.23-0.18 (m, 1H).

### Example 126

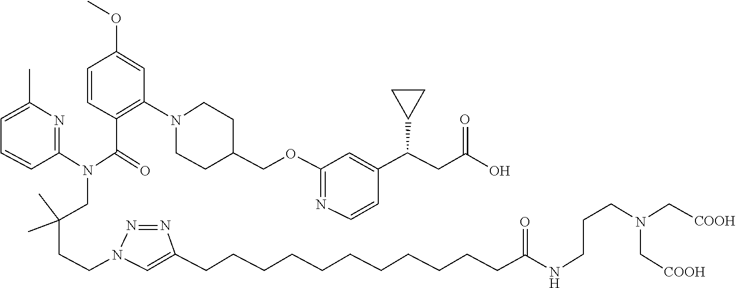 [SMILES: COc1ccc(C(=O)N(CC(C)(C)CCn2cc(CCCCCCCCCCCC(=O)NCCCN(CC(=O)O)CC(=O)O)nn2)c2cccc(C)n2)c(N2CCC(COc3cc([C@@H](CC(=O)O)C4CC4)ccn3)CC2)c1]

MS (ESI) m/z 1066.3 [M+H]⁺. ¹H NMR (400 MHZ, DMSO-d₆): δ 8.04-8.03 (d, 1H), 7.83 (t, NH, 1H), 7.68 (s, 1H), 7.29-7.25 (br, 1H), 7.16-7.14 (d, 1H), 6.92-6.91 (d, 1H), 6.86-6.84 (d, 1H), 6.71 (s, 1H), 6.53-6.51 (d, 1H), 6.47-6.44 (br, 1H), 6.21 (s, 1H), 4.32 (t, 2H), 4.18 (br, 2H), 4.13-4.11 (d, 2H), 4.00 (s, 4H), 3.69 (s, 3H), 3.12-3.04 (m, 4H), 2.69-2.67 (m, 2H), 2.58-2.54 (m, 2H), 2.35 (s, 3H), 2.26-2.20 (m, 1H), 2.07-2.03 (t, 2H), 1.74-1.61 (m, 7H), 1.58-1.55 (m, 2H), 1.50-1.45 (m, 2H), 1.36-1.20 (br, 16H), 1.02-0.98 (m, 1H), 0.78 (s, 6H), 0.52-0.50 (m, 1H), 0.32-0.25 (m, 2H), 0.23-0.18 (m, 1H).

### Example 127

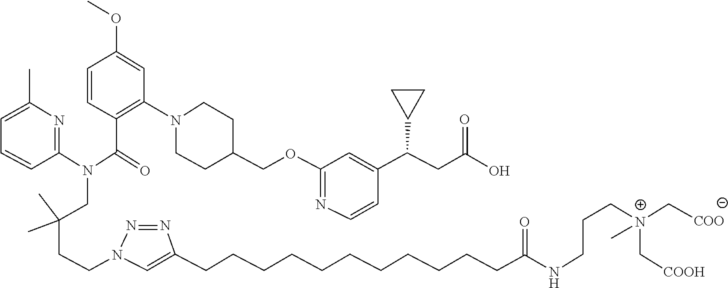

MS (ESI) m/z 540.8 [(M+H)/2]⁺. ¹H NMR (400 MHZ, DMSO-d₆): δ 8.05-8.03 (d, 1H), 7.89 (t, NH, 1H), 7.68 (s, 1H), 7.30-7.25 (br, 1H), 7.16-7.14 (d, 1H), 6.92-6.91 (d, 1H), 6.86-6.84 (d, 1H), 6.71 (s, 1H), 6.54-6.51 (d, 1H), 6.46 (br, 1H), 6.21 (s, 1H), 4.46 (s, 4H), 4.33 (t, 2H), 4.18 (br, 2H), 4.13-4.12 (d, 2H), 3.69 (s, 3H), 3.67-3.63 (m, B, 2H), 3.31 (s, 3H), 3.10-3.07 (m, 2H), 2.69-2.67 (m, 2H), 2.58-2.54 (m, 2H), 2.36 (s, 3H), 2.26-2.21 (m, 1H), 2.05-2.02 (t, 2H), 1.85-1.68 (m, 3H), 1.68-1.60 (m, 4H), 1.55-1.50 (m, 2H), 1.50-1.45 (m, 2H), 1.36-1.20 (br, 16H), 1.02-0.98 (m, 1H), 0.78 (s, 6H), 0.52-0.50 (m, 1H), 0.32-0.25 (m, 2H), 0.23-0.18 (m, 1H).

### Example 128

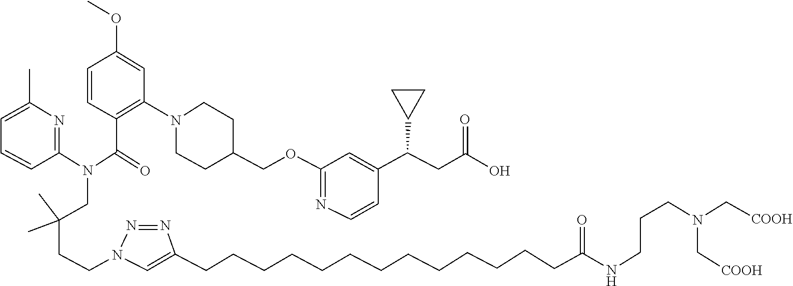 [SMILES: COc1ccc(C(=O)N(CC(C)(C)CCn2cc(CCCCCCCCCCCCCC(=O)NCCCN(CC(=O)O)CC(=O)O)nn2)c2cccc(C)n2)c(N2CCC(COc3cc([C@@H](CC(=O)O)C4CC4)ccn3)CC2)c1]

MS (ESI) m/z 1094.3 [M+H)]⁺. ¹H NMR (400 MHZ, DMSO-d₆): δ 8.05-8.04 (d, 1H), 7.85 (t, NH, 1H), 7.68 (s, 1H), 7.30-7.26 (br, 1H), 7.16-7.14 (d, 1H), 6.93-6.92 (d, 1H), 6.86-6.84 (d, 1H), 6.72 (s, 1H), 6.54-6.51 (d, 1H), 6.47 (br, 1H), 6.21 (s, 1H), 4.32 (t, 2H), 4.18 (br, 2H), 4.13-4.12 (d, 2H), 4.06 (s, 4H), 3.69 (s, 3H), 3.17-3.14 (m, 2H), 3.07-3.04 (m, 2H), 2.69-2.67 (m, 2H), 2.58-2.54 (m, 2H), 2.36 (s, 3H), 2.26-2.20 (m, 1H), 2.05-2.02 (t, 2H), 1.77-1.73 (m, 3H), 1.74-1.61 (m, 7H), 1.58-1.50 (m, 2H), 1.50-1.45 (m, 2H), 1.36-1.20 (br, 20H), 1.02-0.98 (m, 1H), 0.78 (s, 6H), 0.52-0.50 (m, 1H), 0.32-0.25 (m, 2H), 0.23-0.18 (m, 1H).

### Example 129

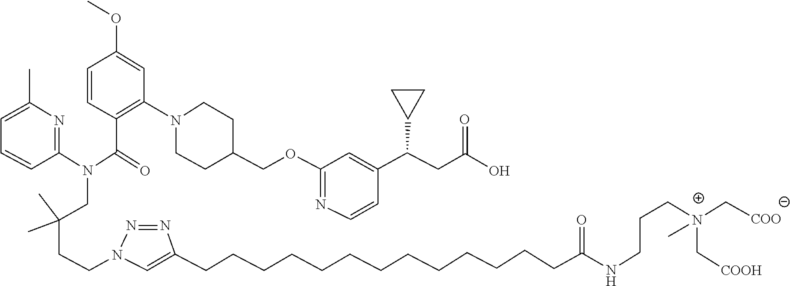

MS (ESI) m/z 554.8 [(M+H)/2]⁺. ¹H NMR (400 MHZ, DMSO-d₆): δ 8.05-8.03 (d, 1H), 7.89 (t, NH, 1H), 7.68 (s, 1H), 7.29-7.25 (br, 1H), 7.16-7.14 (d, 1H), 6.92-6.91 (d, 1H), 6.86-6.84 (d, 1H), 6.72 (s, 1H), 6.53-6.51 (d, 1H), 6.46 (br, 1H), 6.21 (s, 1H), 4.46 (s, 4H), 4.33 (t, 2H), 4.18 (br, 2H), 4.13-4.12 (d, 2H), 3.69 (s, 3H), 3.67-3.63 (m, B, 2H), 3.30 (s, 3H), 3.10-3.07 (m, 2H), 2.69-2.67 (m, 2H), 2.58-2.54 (m, 2H), 2.35 (s, 3H), 2.26-2.20 (m, 1H), 2.05-2.01 (t, 2H), 1.85-1.70 (m, 3H), 1.68-1.58 (m, 4H), 1.55-1.50 (m, 2H), 1.50-1.45 (m, 2H), 1.36-1.20 (br, 20H), 1.02-0.98 (m, 1H), 0.78 (s, 6H), 0.52-0.50 (m, 1H), 0.32-0.25 (m, 2H), 0.23-0.18 (m, 1H).

### Example 130

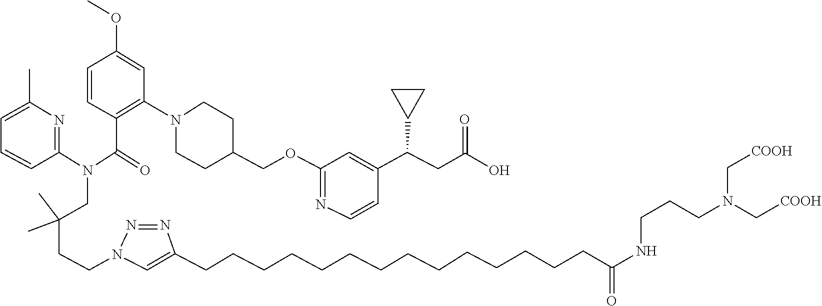 [SMILES: COc1ccc(C(=O)N(CC(C)(C)CCn2cc(CCCCCCCCCCCCCCC(=O)NCCCN(CC(=O)O)CC(=O)O)nn2)c2cccc(C)n2)c(N2CCC(COc3cc([C@@H](CC(=O)O)C4CC4)ccn3)CC2)c1]

MS (ESI) m/z 1108.4 [M+H]⁺.

### Example 131

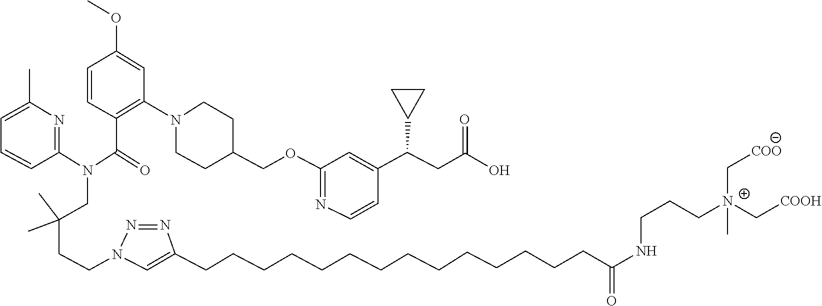

MS (ESI) m/z 561.8 [(M+H)/2]⁺. ¹H NMR (400 MHZ, DMSO-d₆): δ 8.05-8.03 (d, 1H), 7.89 (t, NH, 1H), 7.68 (s, 1H), 7.30-7.26 (br, 1H), 7.16-7.14 (d, 1H), 6.93-6.91 (d, 1H), 6.86-6.84 (d, 1H), 6.72 (s, 1H), 6.54-6.51 (d, 1H), 6.46 (br, 1H), 6.21 (s, 1H), 4.46 (s, 4H), 4.32 (t, 2H), 4.18 (br, 2H), 4.13-4.12 (d, 2H), 3.69 (s, 3H), 3.67-3.63 (m, B, 2H), 3.31 (s, 3H), 3.10-3.06 (m, 2H), 2.69-2.67 (m, 2H), 2.58-2.54 (m, 2H), 2.36 (s, 3H), 2.27-2.21 (m, 1H), 2.05-2.01 (t, 2H), 1.85-1.68 (m, 3H), 1.68-1.59 (m, 4H), 1.55-1.50 (m, 2H), 1.50-1.45 (m, 2H), 1.36-1.20 (br, 22H), 1.02-0.98 (m, 1H), 0.78 (s, 6H), 0.52-0.50 (m, 1H), 0.32-0.25 (m, 2H), 0.23-0.18 (m, 1H).

### Example 132

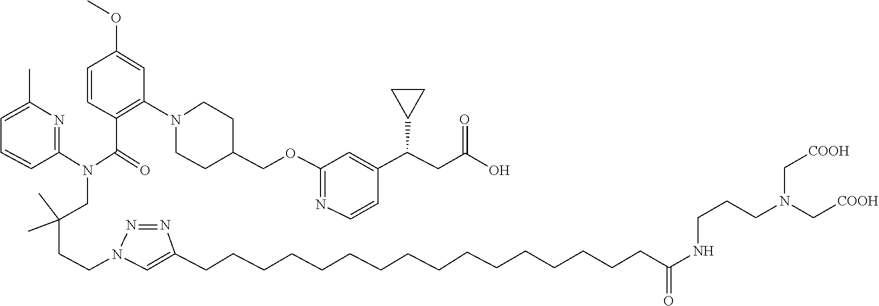 [SMILES: COc1ccc(C(=O)N(CC(C)(C)CCn2cc(CCCCCCCCCCCCCCCCC(=O)NCCCN(CC(=O)O)CC(=O)O)nn2)c2cccc(C)n2)c(N2CCC(COc3cc([C@@H](CC(=O)O)C4CC4)ccn3)CC2)c1]

MS (ESI) m/z 1136.4 [M+H]⁺.

### Example 133

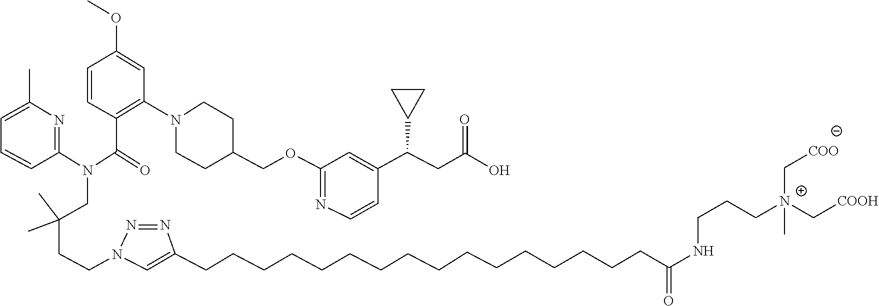

MS (ESI) m/z 1150.5 [M]⁺. ¹H NMR (400 MHZ, DMSO-d₆): δ 8.05-8.04 (d, 1H), 7.89 (t, NH, 1H), 7.68 (s, 1H), 7.29-7.26 (br, 1H), 7.16-7.14 (d, 1H), 6.93-6.91 (d, 1H), 6.86-6.84 (d, 1H), 6.72 (s, 1H), 6.53-6.51 (d, 1H), 6.46 (br, 1H), 6.21 (s, 1H), 4.46 (s, 4H), 4.31 (t, 2H), 4.18 (br, 2H), 4.13-4.12 (d, 2H), 3.69 (s, 3H), 3.67-3.62 (m, B, 2H), 3.31 (s, 3H), 3.10-3.06 (m, 2H), 2.69-2.67 (m, 2H), 2.58-2.54 (m, 2H), 2.36 (s, 3H), 2.27-2.22 (m, 1H), 2.05-2.01 (t, 2H), 1.85-1.68 (m, 3H), 1.68-1.59 (m, 4H), 1.55-1.50 (m, 2H), 1.50-1.45 (m, 2H), 1.36-1.20 (br, 26H), 1.02-0.98 (m, 1H), 0.78 (s, 6H), 0.52-0.50 (m, 1H), 0.32-0.25 (m, 2H), 0.23-0.18 (m, 1H).

### Example 134

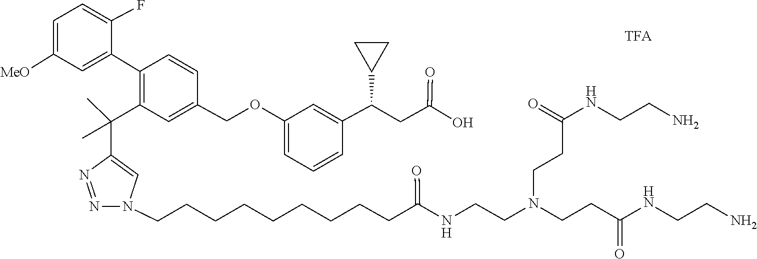 [SMILES: COc1ccc(F)c(-c2ccc(COc3cccc([C@@H](CC(=O)O)C4CC4)c3)cc2C(C)(C)c2cn(CCCCCCCCCC(=O)NCCN(CCC(=O)NCCN)CCC(=O)NCCN)nn2)c1]

MS (ESI) m/z 970.2 [M+H)]⁺. ¹H NMR (400 MHZ, DMSO-d₆): δ 8.43-8.40 (t, NH, 2H), 8.16-8.14 (t, 1H), 7.92 (br, NH₃, 6H), 7.69 (s, 1H), 7.35-7.33 (d, 1H), 7.29 (s, 1H), 7.24-7.20 (m, 1H), 7.00-6.77 (m, 6H), 6.94-5.92 (m, 1H), 5.14 (s, 3H), 4.14-4.01 (m, 2H), 3.59 (s, 3H), 3.42-3.36 (m, 6H), 3.33-3.29 (m, 4H), 3.18-3.16 (m, 2H), 2.90-2.85 (m, 4H), 2.67-2.61 (m, 6H), 2.30-2.24 (m, 1H), 2.10-2.07 (t, 2H), 1.73-1.70 (m, 2H), 1.60 (s, 3H), 1.48-1.44 (m, 2H), 1.46 (s, 3H), 1.30-1.19 (m, 10H), 1.04-0.98 (m, 1H), 0.52-0.48 (m, 1H), 0.33-0.23 (m, 2H), 0.12-0.10 (m, 1H).

### Example 135

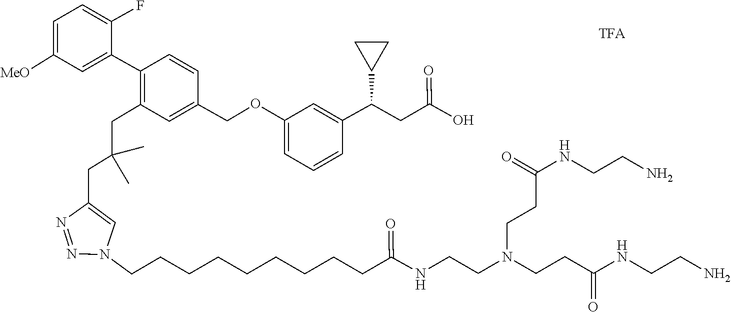 [SMILES: COc1ccc(F)c(-c2ccc(COc3cccc([C@@H](CC(=O)O)C4CC4)c3)cc2CC(C)(C)Cc2cn(CCCCCCCCCC(=O)NCCN(CCC(=O)NCCN)CCC(=O)NCCN)nn2)c1]

MS (ESI) m/z 998.3 [M+H]⁺. ¹H NMR (400 MHZ, DMSO-d₆): δ 8.40 (t, NH, 2H), 8.14 (t, NH, 1H), 7.90 (br s, 6H), 7.69 (s, 1H), 7.45 (s, 1H), 7.39-7.37 (d, 1H), 7.22-7.13 (m, 3H), 6.96-6.91 (m, 2H), 6.86-6.77 (m, 3H), 5.12 (s, 2H), 4.26-4.22 (t, 2H), 3.74 (s, 3H), 3.45-3.36 (m, 6H), 3.33-3.29 (m, 4H), 3.18-3.15 (m, 2H), 2.89-2.86 (m, 4H), 2.71-2.60 (m, 7H), 2.52-2.49 (m, 1H), 2.34 (s, 2H), 2.28-2.22 (m, 1H), 2.09-2.05 (t, 2H), 1.77-1.73 (m, 2H), 1.47-1.44 (m, 2H), 1.30-1.13 (m, 10H), 1.02-0.97 (m, 1H), 0.55-0.50 (m, 6H), 0.52-0.48 (br, 1H), 0.33-0.23 (m, 2H), 0.12-0.10 (m, 1H).

### Example 136

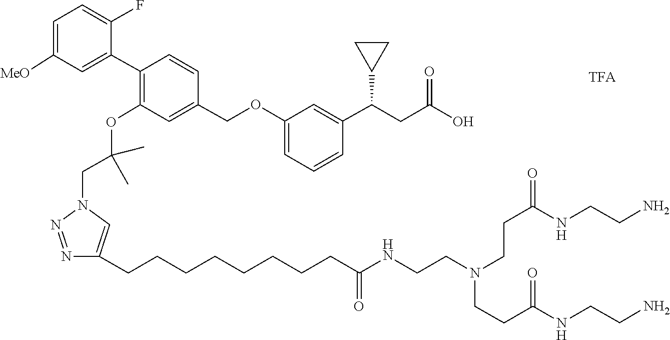 [SMILES: COc1ccc(F)c(-c2ccc(COc3cccc([C@@H](CC(=O)O)C4CC4)c3)cc2OC(C)(C)Cn2cc(CCCCCCCCC(=O)NCCN(CCC(=O)NCCN)CCC(=O)NCCN)nn2)c1]

MS (ESI) m/z 986.2 [M+H]⁺. ¹H NMR (400 MHZ, DMSO-d₆): δ 8.41 (t, NH, 2H), 8.14 (t, NH, 1H), 7.90 (br, 6H), 7.32-7.30 (d, 1H), 7.24-7.16 (m, 4H), 7.12 (s, 1H), 6.97-6.93 (m, 1H), 6.91-6.88 (m, 2H), 6.85-6.83 (m, 2H), 5.13 (s, 2H), 4.30 (s, 2H), 3.76 (s, 3H), 3.42-3.38 (m, 6H), 3.33-3.29 (m, 4H), 3.18-3.15 (m, 2H), 2.89-2.87 (m, 4H), 2.69-2.61 (m, 6H), 2.54-2.50 (t, 2H), 2.28-2.22 (m, 1H), 2.10-2.06 (t, 2H), 1.51-1.46 (m, 4H), 1.30-1.20 (m, 8H), 1.06 (s, 6H), 1.01-0.96 (m, 1H), 0.49-0.46 (m, 1H), 0.33-0.20 (m, 2H), 0.24-0.20 (m, 1H).

### Example 137

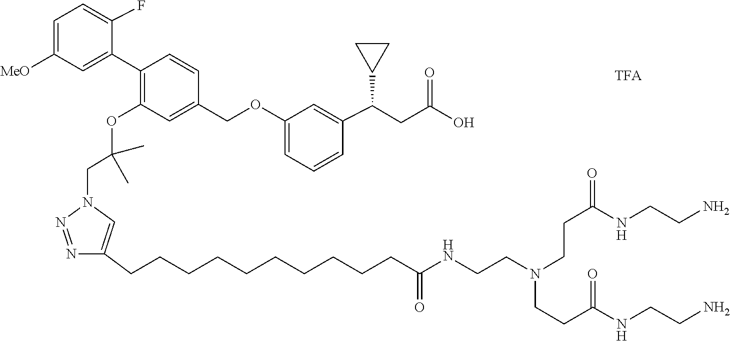 [SMILES: COc1ccc(F)c(-c2ccc(COc3cccc([C@@H](CC(=O)O)C4CC4)c3)cc2OC(C)(C)Cn2cc(CCCCCCCCCCC(=O)NCCN(CCC(=O)NCCN)CCC(=O)NCCN)nn2)c1]

MS (ESI) m/z 1014.3 [M+H)]⁺. ¹H NMR (400 MHZ, DMSO-d₆): δ 8.41 (t, NH, 2H), 8.14 (t, NH, 1H), 7.90 (br, 6H), 7.32-7.30 (d, 1H), 7.24-7.15 (m, 4H), 7.12 (s, 1H), 6.97-6.93 (m, 1H), 6.91-6.88 (m, 2H), 6.85-6.83 (m, 2H), 5.13 (s, 2H), 4.30 (s, 2H), 3.75 (s, 3H), 3.42-3.36 (m, 6H), 3.33-3.29 (m, 4H), 3.18-3.15 (m, 2H), 2.90-2.86 (m, 4H), 2.69-2.61 (m, 6H), 2.54-2.50 (t, 2H), 2.28-2.22 (m, 1H), 2.10-2.06 (t, 2H), 1.51-1.46 (m, 4H), 1.30-1.20 (m, 12H), 1.06 (s, 6H), 1.01-0.96 (m, 1H), 0.49-0.46 (m, 1H), 0.33-0.20 (m, 2H), 0.24-0.20 (m, 1H).

### Example 138

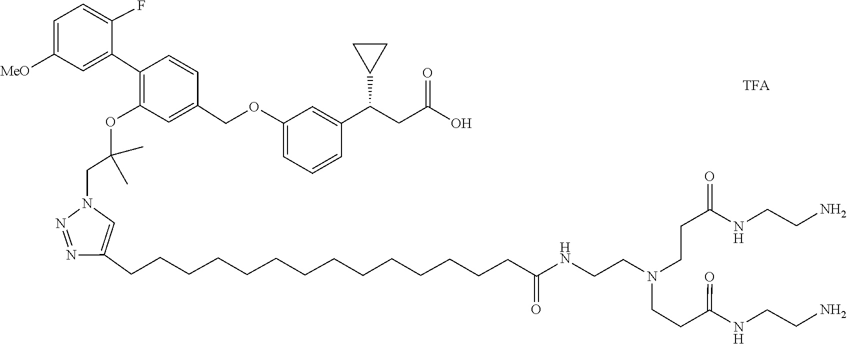 [SMILES: COc1ccc(F)c(-c2ccc(COc3cccc([C@@H](CC(=O)O)C4CC4)c3)cc2OC(C)(C)Cn2cc(CCCCCCCCCCCCCCC(=O)NCCN(CCC(=O)NCCN)CCC(=O)NCCN)nn2)c1]

MS (ESI) m/z 535.8 [(M+2H)/2]⁺. ¹H NMR (400 MHZ, DMSO-d₆): δ 8.41 (t, NH, 2H), 8.14 (t, NH, 1H), 7.90 (br, 6H), 7.32-7.30 (d, 1H), 7.23-7.15 (m, 4H), 7.12 (s, 1H), 6.97-6.93 (m, 1H), 6.91-6.88 (m, 2H), 6.85-6.82 (m, 2H), 5.13 (s, 2H), 4.34 (s, 2H), 3.75 (s, 3H), 3.42-3.36 (m, 6H), 3.33-3.29 (m, 4H), 3.18-3.15 (m, 2H), 2.90-2.86 (m, 4H), 2.69-2.61 (m, 6H), 2.54-2.50 (t, 2H), 2.28-2.22 (m, 1H), 2.10-2.06 (t, 2H), 1.51-1.46 (m, 4H), 1.30-1.20 (m, 20H), 1.06 (s, 6H), 1.01-0.96 (m, 1H), 0.49-0.46 (m, 1H), 0.33-0.20 (m, 2H), 0.24-0.20 (m, 1H).

### Example 139

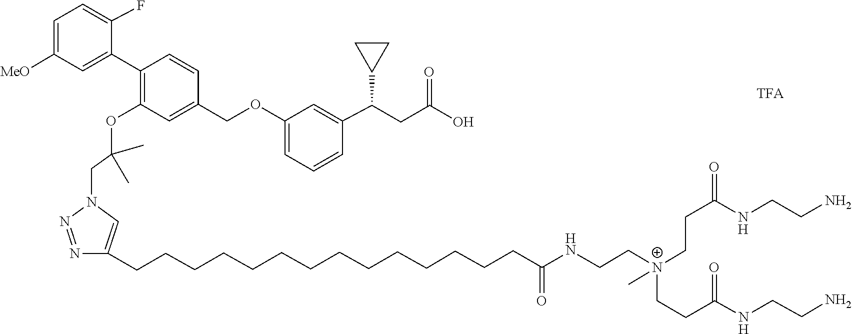 [SMILES: COc1ccc(F)c(-c2ccc(COc3cccc([C@@H](CC(=O)O)C4CC4)c3)cc2OC(C)(C)Cn2cc(CCCCCCCCCCCCCCC(=O)NCC[N+](C)(CCC(=O)NCCN)CCC(=O)NCCN)nn2)c1]

MS (ESI) m/z 1084.4 [M]⁺. ¹H NMR (400 MHZ, DMSO-d₆): δ 8.39 (t, NH, 2H), 8.16 (t, NH, 1H), 7.91 (br, 6H), 7.32-7.30 (d, 1H), 7.24-7.15 (m, 4H), 7.12 (s, 1H), 6.96-6.93 (m, 1H), 6.91-6.88 (m, 2H), 6.84-6.82 (m, 2H), 5.13 (s, 2H), 4.30 (s, 2H), 3.75 (s, 3H), 3.58-3.55 (m, 4H), 3.44-3.40 (m, 2H), 3.34-3.29 (m, 6H), 3.18-3.15 (m, 2H), 3.03 (s, 3H), 2.90-2.86 (m, 4H), 2.67-2.60 (m, 6H), 2.54-2.50 (t, 2H), 2.28-2.22 (m, 1H), 2.10-2.06 (t, 2H), 1.51-1.46 (m, 4H), 1.30-1.20 (m, 20H), 1.06 (s, 6H), 1.01-0.96 (m, 1H), 0.49-0.46 (m, 1H), 0.33-0.20 (m, 2H), 0.24-0.20 (m, 1H).

### Example 140

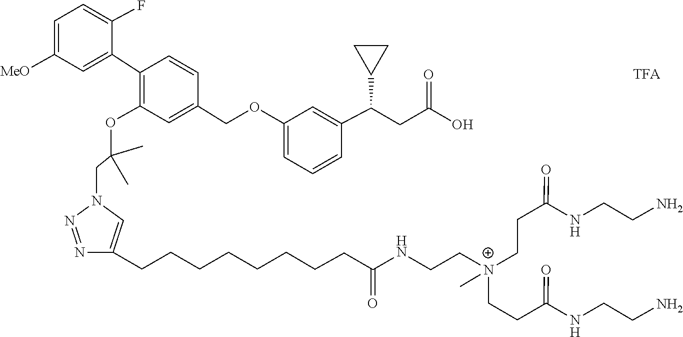 [SMILES: COc1ccc(F)c(-c2ccc(COc3cccc([C@@H](CC(=O)O)C4CC4)c3)cc2OC(C)(C)Cn2cc(CCCCCCCCC(=O)NCC[N+](C)(CCC(=O)NCCN)CCC(=O)NCCN)nn2)c1]

MS (ESI) m/z 1000.2 [M]⁺. ¹H NMR (400 MHZ, DMSO-d₆): δ 8.40 (t, NH, 2H), 8.18 (t, NH, 1H), 7.93 (br, 6H), 7.32-7.30 (d, 1H), 7.24-7.16 (m, 4H), 7.12 (s, 1H), 6.98-6.94 (m, 1H), 6.92-6.88 (m, 2H), 6.86-6.83 (m, 2H), 5.13 (s, 2H), 4.31 (s, 2H), 3.76 (s, 3H), 3.58-3.55 (m, 4H), 3.44-3.40 (m, 2H), 3.34-3.29 (m, 6H), 3.03 (s, 3H), 2.90-2.86 (m, 4H), 2.70-2.58 (m, 6H), 2.54-2.50 (t, 2H), 2.28-2.22 (m, 1H), 2.11-2.07 (t, 2H), 1.51-1.46 (m, 4H), 1.30-1.20 (m, 8H), 1.07 (s, 6H), 1.01-0.96 (m, 1H), 0.49-0.46 (m, 1H), 0.33-0.20 (m, 2H), 0.24-0.20 (m, 1H).

### Example 141

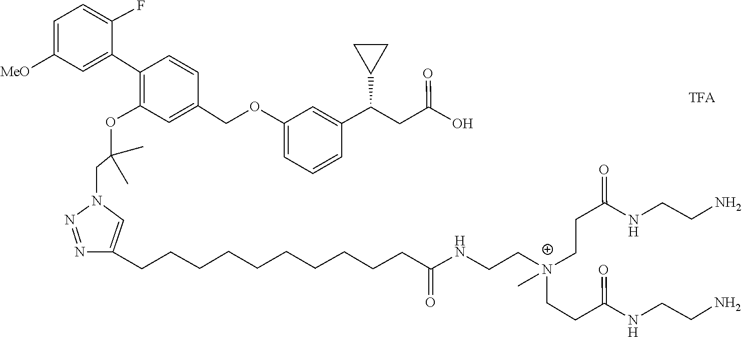 [SMILES: COc1ccc(F)c(-c2ccc(COc3cccc([C@@H](CC(=O)O)C4CC4)c3)cc2OC(C)(C)Cn2cc(CCCCCCCCCCC(=O)NCC[N+](C)(CCC(=O)NCCN)CCC(=O)NCCN)nn2)c1]

MS (ESI) m/z 1028.3 [M]⁺. ¹H NMR (400 MHZ, DMSO-d₆): δ 8.40 (t, NH, 2H), 8.17 (t, NH, 1H), 7.93 (br, 6H), 7.32-7.30 (d, 1H), 7.24-7.15 (m, 4H), 7.12 (s, 1H), 6.97-6.93 (m, 1H), 6.91-6.88 (m, 2H), 6.84-6.82 (m, 2H), 5.13 (s, 2H), 4.30 (s, 2H), 3.75 (s, 3H), 3.58-3.55 (m, 4H), 3.44-3.40 (m, 2H), 3.34-3.29 (m, 6H), 3.03 (s, 3H), 2.90-2.86 (m, 4H), 2.70-2.58 (m, 6H), 2.54-2.50 (t, 2H), 2.28-2.22 (m, 1H), 2.10-2.06 (t, 2H), 1.51-1.46 (m, 4H), 1.30-1.20 (m, 12H), 1.06 (s, 6H), 1.01-0.96 (m, 1H), 0.49-0.46 (m, 1H), 0.33-0.20 (m, 2H), 0.24-0.20 (m, 1H).

### Example 142

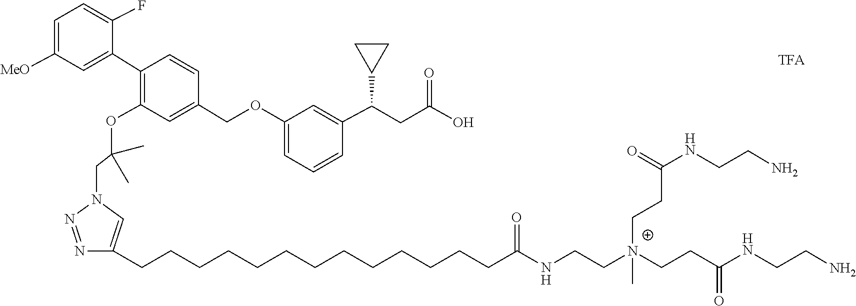 [SMILES: COc1ccc(F)c(-c2ccc(COc3cccc([C@@H](CC(=O)O)C4CC4)c3)cc2OC(C)(C)Cn2cc(CCCCCCCCCCCCCC(=O)NCC[N+](C)(CCC(=O)NCCN)CCC(=O)NCCN)nn2)c1]

MS (ESI) m/z 1070.3 [M]⁺. ¹H NMR (400 MHZ, DMSO-d₆): δ 8.40 (t, NH, 2H), 8.17 (t, NH, 1H), 7.93 (br, 6H), 7.32-7.30 (d, 1H), 7.24-7.15 (m, 4H), 7.12 (s, 1H), 6.97-6.93 (m, 1H), 6.91-6.88 (m, 2H), 6.84-6.82 (m, 2H), 5.13 (s, 2H), 4.30 (s, 2H), 3.75 (s, 3H), 3.58-3.55 (m, 4H), 3.44-3.40 (m, 2H), 3.34-3.29 (m, 6H), 3.03 (s, 3H), 2.90-2.86 (m, 4H), 2.70-2.58 (m, 6H), 2.54-2.50 (t, 2H), 2.28-2.22 (m, 1H), 2.10-2.06 (t, 2H), 1.51-1.46 (m, 4H), 1.30-1.20 (m, 18H), 1.06 (s, 6H), 1.01-0.96 (m, 1H), 0.49-0.46 (m, 1H), 0.33-0.20 (m, 2H), 0.24-0.20 (m, 1H).

### Example 143

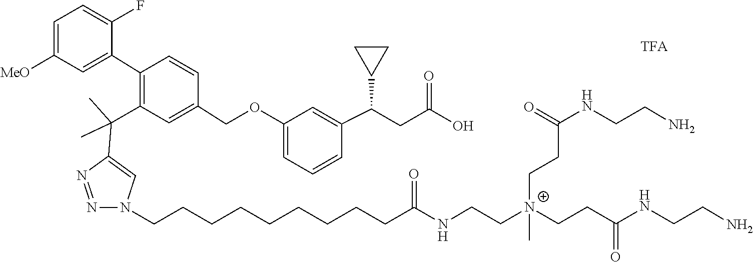 [SMILES: COc1ccc(F)c(-c2ccc(COc3cccc([C@@H](CC(=O)O)C4CC4)c3)cc2C(C)(C)c2cn(CCCCCCCCCC(=O)NCC[N+](C)(CCC(=O)NCCN)CCC(=O)NCCN)nn2)c1]

MS (ESI) m/z 985.3 [M]⁺. ¹H NMR (400 MHZ, DMSO-d₆): δ 8.42-8.40 (t, NH, 2H), 8.20-8.17 (t, 1H), 7.94 (br, NH₃, 6H), 7.70 (s, 1H), 7.36-7.34 (d, 1H), 7.30 (s, 1H), 7.25-7.21 (m, 1H), 7.00-6.78 (m, 6H), 6.95-5.93 (m, 1H), 5.14 (s, 3H), 4.13-4.03 (m, 2H), 3.63-3.60 (m, 4H), 3.59 (s, 3H), 3.47-3.44 (m, 2H), 3.34-3.30 (m, 6H), 3.03 (s, 3H), 2.90-2.85 (m, 4H), 2.70-2.63 (m, 6H), 2.28-2.24 (m, 1H), 2.11-2.08 (t, 2H), 1.73-1.70 (m, 2H), 1.59 (s, 3H), 1.48-1.44 (m, 2H), 1.44 (s, 3H), 1.30-1.19 (m, 10H), 1.04-0.98 (m, 1H), 0.52-0.48 (m, 1H), 0.33-0.23 (m, 2H), 0.12-0.10 (m, 1H).

### Example 144

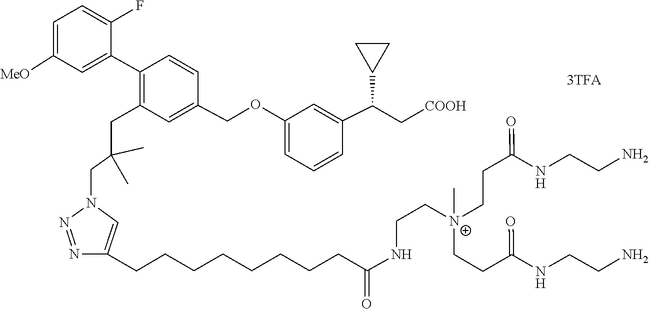 [SMILES: COc1ccc(F)c(-c2ccc(COc3cccc([C@@H](CC(=O)O)C4CC4)c3)cc2CC(C)(C)Cn2cc(CCCCCCCCC(=O)NCC[N+](C)(CCC(=O)NCCN)CCC(=O)NCCN)nn2)c1]

MS (ESI) m/z 998.2 [M]⁺. ¹H NMR (400 MHZ, DMSO-d₆): δ 8.41 (t, NH, 2H), 8.18 (t, NH, 1H), 7.94 (br, 6H), 7.53 (s, 1H), 7.44 (s, 1H), 7.42-7.40 (d, 1H), 7.24-7.12 (m, 3H), 6.95-6.92 (m, 2H), 6.87-6.82 (m, 2H), 6.80-6.77 (m, 1H), 5.14 (s, 2H), 3.98 (s, 2H), 3.74 (s, 3H), 3.60-3.56 (m, 4H), 3.47-3.45 (m, 2H), 3.34-3.31 (m, 6H), 3.04 (s, 3H), 2.90-2.86 (m, 4H), 2.73-2.61 (m, 7H), 2.57-2.51 (t, 3H), 2.28-2.22 (m, 1H), 2.11-2.07 (t, 2H), 1.55-1.47 (m, 4H), 1.28-1.23 (m, 8H), 1.00-0.96 (m, 1H), 0.56-0.47 (br, 7H), 0.33-0.23 (m, 2H), 0.12-0.10 (m, 1H).

### Example 145

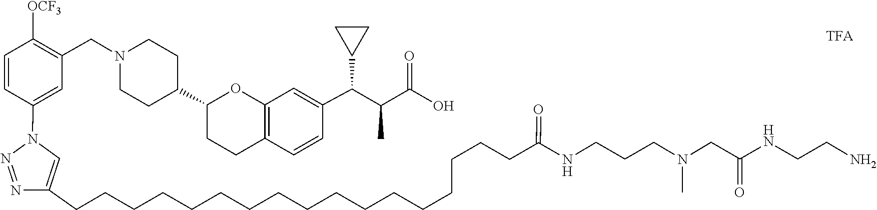 [SMILES: C[C@H](C(=O)O)[C@H](c1ccc2c(c1)O[C@@H](C1CCN(Cc3cc(-n4cc(CCCCCCCCCCCCCCCCCC(=O)NCCCN(C)CC(=O)NCCN)nn4)ccc3OC(F)(F)F)CC1)CC2)C1CC1]

MS (ESI) m/z 1038.3 [M+H]⁺. ¹H NMR (400 MHZ, DMSO-d₆): δ 8.78 (t, NH, 2H), 8.60 (s, 1H), 8.40 (s, 1H), 8.11-8.09 (d, 1H), 7.94 (br, 4H), 7.75-7.73 (d, 1H), 6.97-6.95 (d, 1H), 6.66-6.64 (d, 1H), 6.51 (s, 1H), 4.48 (s, 2H), 3.91 (br, 1H), 3.85-3.82 (m, 2H), 3.41-3.37 (m, 4H), 3.15-3.07 (m, 6H), 3.23-3.13 (m, 4H), 2.94-2.89 (m, 2H), 2.80 (s, 3H), 2.73-2.60 (m, 5H), 2.07-2.03 (t, 2H), 2.05-1.65 (m, 12H), 1.52-1.48 (m, 2H), 1.37-1.26 (m, 26H), 1.06-1.02 (m, 1H), 0.82-0.80 (d, 3H), 0.53-0.49 (m, 1H), 0.28-0.19 (m, 2H), −0.04 ˜−0.08 (m, 1H).

### Example 146

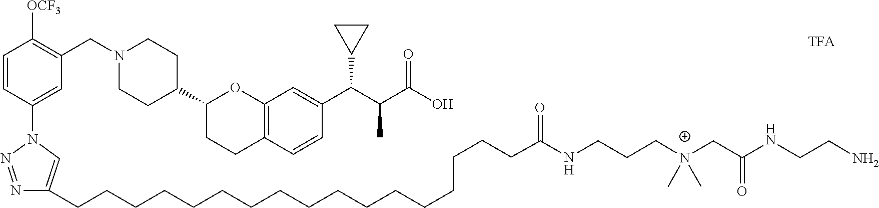 [SMILES: C[C@H](C(=O)O)[C@H](c1ccc2c(c1)O[C@@H](C1CCN(Cc3cc(-n4cc(CCCCCCCCCCCCCCCCCC(=O)NCCC[N+](C)(C)CC(=O)NCCN)nn4)ccc3OC(F)(F)F)CC1)CC2)C1CC1]

MS (ESI) m/z 1051.3 [M]⁺. ¹H NMR (400 MHZ, DMSO-d₆): δ 8.88 (t, NH, 2H), 8.60 (s, 1H), 8.38 (s, 1H), 8.09-8.08 (d, 1H), 7.94 (br, 4H), 7.73-7.71 (d, 1H), 6.97-6.95 (d, 1H), 6.66-6.64 (d, 1H), 6.52 (s, 1H), 4.46 (br, 2H), 4.03 (s, 2H), 3.82-3.79 (m, 1H), 3.47-3.43 (m, 2H), 3.37-3.30 (m, 4H), 3.18 (s, 6H), 3.11-3.03 (m, 4H), 2.94-2.90 (s, 2H), 2.76-2.60 (m, 5H), 2.07-2.03 (t, 2H), 2.05-1.65 (m, 12H), 1.52-1.48 (m, 2H), 1.37-1.26 (m, 26H), 1.06-1.02 (m, 1H), 0.82-0.80 (d, 3H), 0.53-0.49 (m, 1H), 0.28-0.19 (m, 2H), −0.04 ˜−0.08 (m, 1H).

### Example 147

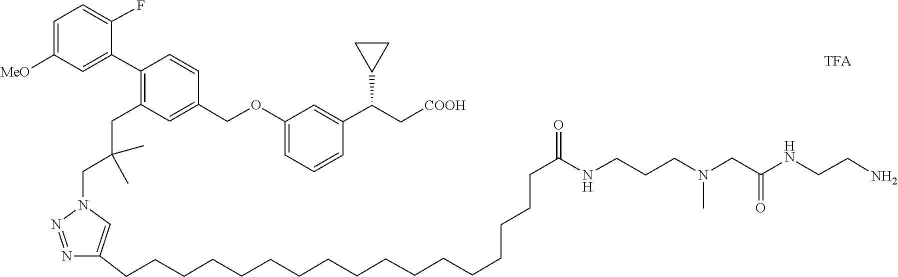 [SMILES: COc1ccc(F)c(-c2ccc(COc3cccc([C@@H](CC(=O)O)C4CC4)c3)cc2CC(C)(C)Cn2cc(CCCCCCCCCCCCCCCCCC(=O)NCCCN(C)CC(=O)NCCN)nn2)c1]

MS (ESI) m/z 1010.3 [M+H]⁺. ¹H NMR (400 MHz, DMSO-d₆): δ 8.80 (t, NH, 2H), 7.95 (br, NH, 4H), 7.62 (s, 1H), 7.44 (s, 1H), 7.42-7.40 (d, 1H), 7.23-7.12 (m, 3H), 6.95-6.91 (m, 2H), 6.86-6.82 (m, 2H), 6.80-6.77 (m, 1H), 5.14 (s, 2H), 3.98 (s, 2H), 3.90-3.86 (br, 2H), 3.74 (s, 3H), 3.41-3.37 (m, 2H), 3.10-3.05 (m, 4H), 2.95-2.90 (m, 2H), 2.80 (s, 3H), 2.73-2.54 (m, 6H), 2.28-2.22 (m, 1H), 2.07-2.03 (t, 2H), 1.81-1.76 (m, 2H), 1.55-1.47 (m, 4H), 1.31-1.19 (m, 26H), 1.00-0.96 (m, 1H), 0.56-0.47 (br, 7H), 0.33-0.23 (m, 2H), 0.12-0.10 (m, 1H).

### Example 148

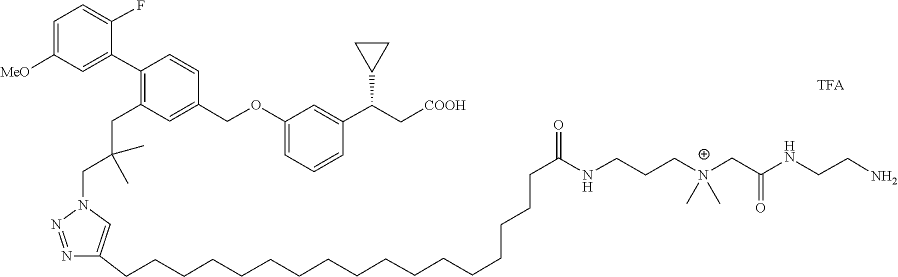 [SMILES: COc1ccc(F)c(-c2ccc(COc3cccc([C@@H](CC(=O)O)C4CC4)c3)cc2CC(C)(C)Cn2cc(CCCCCCCCCCCCCCCCCC(=O)NCCC[N+](C)(C)CC(=O)NCCN)nn2)c1]

MS (ESI) m/z 1025.4 [M]⁺. ¹H NMR (400 MHz, DMSO-d₆): δ 8.87 (t, NH, 2H), 7.94 (br, NH, 4H), 7.62 (s, 1H), 7.44 (s, 1H), 7.41-7.40 (d, 1H), 7.23-7.12 (m, 3H), 6.95-6.91 (m, 2H), 6.86-6.82 (m, 2H), 6.80-6.77 (m, 1H), 5.14 (s, 2H), 4.03 (s, 2H), 3.98 (s, 2H), 3.74 (s, 3H), 3.47-3.43 (m, 2H), 3.39-3.35 (m, 2H), 3.18 (m, 6H), 3.10-3.06 (m, 2H), 2.95-2.90 (m, 2H), 2.73-2.54 (m, 4H), 2.55-2.50 (m, 3H), 2.28-2.22 (m, 1H), 2.07-2.03 (t, 2H), 1.87-1.79 (m, 2H), 1.56-1.47 (m, 4H), 1.31-1.19 (m, 26H), 1.01-0.96 (m, 1H), 0.56-0.47 (br, 7H), 0.33-0.23 (m, 2H), 0.12-0.10 (m, 1H).

### Example 149

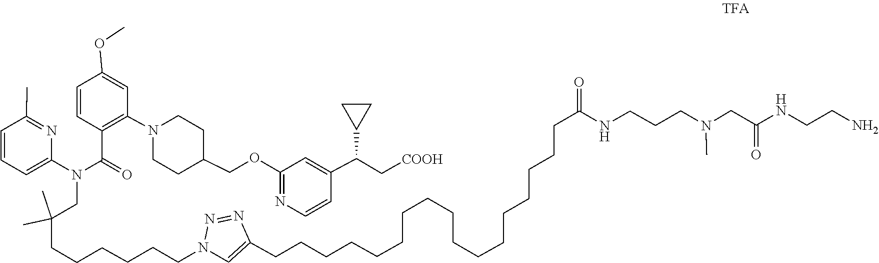 [SMILES: COc1ccc(C(=O)N(CC(C)(C)CCCCCCn2cc(CCCCCCCCCCCCCCCCCC(=O)NCCCN(C)CC(=O)NCCN)nn2)c2cccc(C)n2)c(N2CCC(COc3cc([C@@H](CC(=O)O)C4CC4)ccn3)CC2)c1]

MS (ESI) m/z 1025.5 [M+H]⁺. ¹H NMR (400 MHZ, DMSO-d₆): δ 8.78 (t, NH, 1H), 7.94 (t, NH, 1H), 7.93 (br, NH₃, 3H), 8.05-8.04 (d, 1H), 7.80 (s, 1H), 7.26 (br, 1H), 7.11-7.09 (d, 1H), 6.93-6.92 (d, 1H), 6.85-6.83 (d, 1H), 6.72 (s, 1H), 6.53-6.50 (d, 1H), 6.41 (br, 1H), 6.22 (s, 1H), 4.24-4.21 (t, 2H), 4.14-4.12 (d, 2H), 4.09 (br, 2H), 3.92-3.86 (br, 2H), 3.69 (s, 3H), 3.41-3.37 (m, 2H), 3.09-3.05 (m, 4H), 2.94-2.90 (m, 2H), 2.80 (s, 3H), 2.70-2.68 (d, 2H), 2.59-2.56 (t, 2H), 2.33 (s, 3H), 2.26-2.22 (m, 1H), 2.07-2.03 (t, 2H), 1.79-1.75 (m, 3H), 1.71-1.66 (m, 4H), 1.56-1.54 (m, 2H), 1.49-1.46 (m, 2H), 1.39 (br, 2H), 1.25 (br, 26H), 1.25-0.94 (m, 9H), 0.75 (s, 6H), 0.53-0.50 (m, 1H), 0.37-0.20 (m, 2H), 0.17-0.13 (m, 1H).

### Example 150

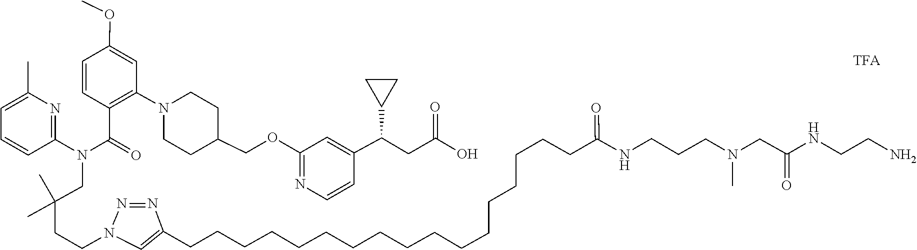 [SMILES: COc1ccc(C(=O)N(CC(C)(C)CCn2cc(CCCCCCCCCCCCCCCCCC(=O)NCCCN(C)CC(=O)NCCN)nn2)c2cccc(C)n2)c(N2CCC(COc3cc([C@@H](CC(=O)O)C4CC4)ccn3)CC2)c1]

MS (ESI) m/z 1149.5 [M+H]⁺. ¹H NMR (400 MHZ, DMSO-d₆): δ 8.78 (t, NH, 1H), 8.06-8.04 (d, 1H), 7.94 (t, NH, 1H), 7.93 br, NH₃, 3H), 7.69 (s, 1H), 7.30-7.26 (br, 1H), 7.17-6.14 (d, 1H), 6.94-6.93 (d, 1H), 6.86-6.85 (d, 1H), 6.74 (s, 1H), 6.54-6.51 (d, 1H), 6.47 (br, 1H), 6.22 (s, 1H), 4.35-4.33 (t, 2H), 4.19 (br, 2H), 4.14-4.12 (d, 2H), 3.92-3.83 (br, 2H), 3.69 (s, 3H), 3.41-3.37 (m, 2H), 3.08-3.07 (m, 4H), 2.94-2.90 (m, 2H), 2.80 (s, 3H), 2.70-2.68 (d, 2H), 2.59-2.55 (m, 2H), 2.36 (s, 3H), 2.27-2.21 (m, 1H), 2.10-2.03 (t, 2H), 1.97-1.77 (m, 3H), 1.69-1.66 (m, 5H), 1.55-1.30 (m, 6H), 1.25 (br, 26H), 1.02-0.98 (m, 1H), 0.80 (s, 6H), 0.52-0.50 (m, 1H), 0.32-0.25 (m, 2H), 0.23-0.18 (m, 1H).

### Example 151

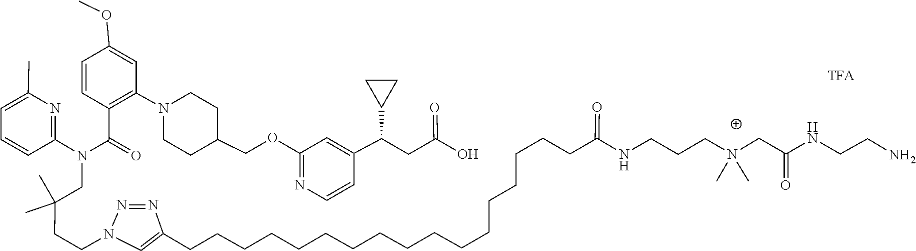

MS (ESI) m/z 1163.4 [M]⁺. ¹H NMR (400 MHZ, DMSO-d₆): δ 8.84 (t, NH, 1H), 8.05-8.04 (d, 1H), 7.94 (t, NH, 1H), 7.93 br, NH₃, 3H), 7.68 (s, 1H), 7.30-7.26 (br, 1H), 7.16-6.14 (d, 1H), 6.93-6.92 (d, 1H), 6.86-6.84 (d, 1H), 6.73 (s, 1H), 6.54-6.51 (d, 1H), 6.46 (br, 1H), 6.22 (s, 1H), 4.33 (t, 2H), 4.18 (br, 2H), 4.14-4.12 (d, 2H), 4.02 (br, 2H), 3.69 (s, 3H), 3.47-3.43 (m, 2H), 3.38-3.36 (m, 2H), 3.18 (s, 6H), 3.09-3.07 (m, 2H), 2.93-2.90 (m, 2H), 2.70-2.68 (d, 2H), 2.58-2.55 (m, 2H), 2.36 (s, 3H), 2.27-2.21 (m, 1H), 2.07-2.03 (t, 2H), 1.82-1.72 (m, 3H), 1.69-1.66 (m, 4H), 1.55-1.30 (m, 6H), 1.25 (br, 26H), 1.02-0.98 (m, 1H), 0.80 (s, 6H), 0.52-0.50 (m, 1H), 0.32-0.25 (m, 2H), 0.23-0.18 (m, 1H).

### Example 152

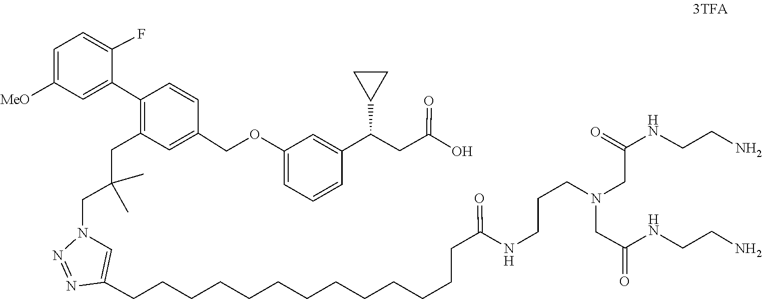 [SMILES: COc1ccc(F)c(-c2ccc(COc3cccc([C@@H](CC(=O)O)C4CC4)c3)cc2CC(C)(C)Cn2cc(CCCCCCCCCCCCCC(=O)NCCCN(CC(=O)NCCN)CC(=O)NCCN)nn2)c1]

MS (ESI) m/z 1040.4 [M+H]⁺. ¹H NMR (400 MHZ, DMSO-d₆): δ 8.62 (t, NH, 2H), 7.93 (br, 6H), 7.88 (t, NH, 1H), 7.62 (s, 1H), 7.44 (s, 1H), 7.41-7.39 (d, 1H), 7.23-7.16 (m, 3H), 6.94-6.91 (m, 2H), 6.86-6.81 (m, 2H), 6.79-6.76 (m, 1H), 5.13 (s, 2H), 3.97 (s, 2H), 3.73 (s, 3H), 3.80 (br, 4H), 3.39-3.34 (m, 4H), 3.08-3.03 (m, 2H), 2.97-2.88 (m, 6H), 2.73-2.67 (m, 1H), 2.66-2.59 (m, 2H), 2.57-2.51 (m, 3H), 2.28-2.21 (m, 1H), 2.05-2.01 (t, 2H), 1.71-1.65 (m, 2H), 1.56-1.52 (m, 2H), 1.47-1.41 (m, 2H), 1.29-1.21 (m, 18H), 1.01-0.97 (m, 1H), 0.58-0.45 (m, 7H), 0.33-0.23 (m, 2H), 0.12-0.10 (m, 1H).

### Example 153

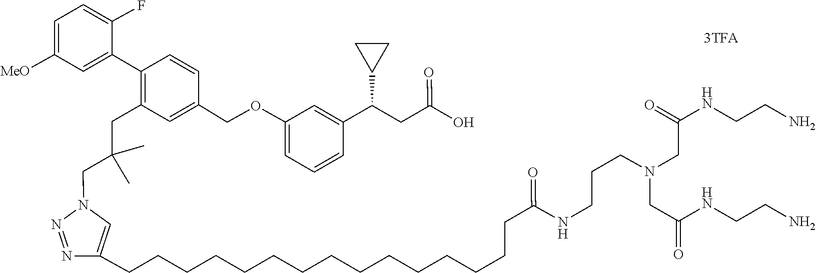 [SMILES: COc1ccc(F)c(-c2ccc(COc3cccc([C@@H](CC(=O)O)C4CC4)c3)cc2CC(C)(C)Cn2cc(CCCCCCCCCCCCCCCC(=O)NCCCN(CC(=O)NCCN)CC(=O)NCCN)nn2)c1]

MS (ESI) m/z 1068.4 [M+H]⁺. ¹H NMR (400 MHZ, DMSO-d₆): δ 8.64 (t, NH, 2H), 7.94 (br, 6H), 7.88 (t, NH, 1H), 7.62 (s, 1H), 7.43 (s, 1H), 7.41-7.39 (d, 1H), 7.23-7.11 (m, 3H), 6.94-6.91 (m, 2H), 6.86-6.81 (m, 2H), 6.79-6.76 (m, 1H), 5.13 (s, 2H), 3.97 (s, 2H), 3.73 (s, 3H), 3.80 (br, 4H), 3.39-3.34 (m, 4H), 3.08-3.03 (m, 2H), 2.98 (br, 2H), 2.91-2.88 (m, 4H), 2.73-2.67 (m, 1H), 2.66-2.59 (m, 2H), 2.57-2.51 (m, 3H), 2.28-2.21 (m, 1H), 2.05-2.01 (t, 2H), 1.71-1.65 (m, 2H), 1.56-1.52 (m, 2H), 1.47-1.44 (m, 2H), 1.29-1.21 (m, 22H), 1.01-0.97 (m, 1H), 0.58-0.45 (m, 7H), 0.33-0.23 (m, 2H), 0.12-0.10 (m, 1H).

### Example 154

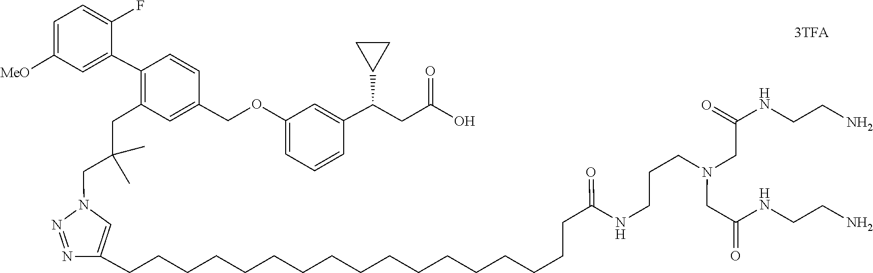 [SMILES: COc1ccc(F)c(-c2ccc(COc3cccc([C@@H](CC(=O)O)C4CC4)c3)cc2CC(C)(C)Cn2cc(CCCCCCCCCCCCCCCCCC(=O)NCCCN(CC(=O)NCCN)CC(=O)NCCN)nn2)c1]

MS (ESI) m/z 1096.5 [M+H]⁺. ¹H NMR (400 MHZ, DMSO-d₆): δ 8.60 (t, NH, 2H), 7.93 (br, 6H), 7.87 (t, NH, 1H), 7.62 (s, 1H), 7.43 (s, 1H), 7.41-7.39 (d, 1H), 7.23-7.11 (m, 3H), 6.94-6.91 (m, 2H), 6.86-6.81 (m, 2H), 6.79-6.76 (m, 1H), 5.13 (s, 2H), 3.97 (s, 2H), 3.73 (s, 3H), 3.80 (br, 4H), 3.38-3.34 (m, 4H), 3.08-3.03 (m, 2H), 2.92-2.88 (m, 6H), 2.73-2.67 (m, 1H), 2.66-2.59 (m, 2H), 2.57-2.51 (m, 3H), 2.28-2.21 (m, 1H), 2.05-2.01 (t, 2H), 1.71-1.65 (m, 2H), 1.56-1.52 (m, 2H), 1.47-1.44 (m, 2H), 1.29-1.21 (m, 26H), 1.01-0.97 (m, 1H), 0.58-0.45 (m, 7H), 0.33-0.23 (m, 2H), 0.12-0.10 (m, 1H).

### Example 155

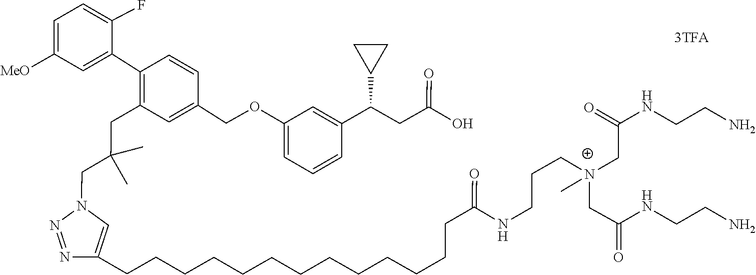 [SMILES: COc1ccc(F)c(-c2ccc(COc3cccc([C@@H](CC(=O)O)C4CC4)c3)cc2CC(C)(C)Cn2cc(CCCCCCCCCCCCCC(=O)NCCC[N+](C)(CC(=O)NCCN)CC(=O)NCCN)nn2)c1]

MS (ESI) m/z 527.8 [(M+H)/2]⁺. ¹H NMR (400 MHZ, DMSO-d₆): δ 8.96 (t, NH, 2H), 8.06-7.94 (br, 8H), 7.63 (s, 1H), 7.44 (s, 1H), 7.42-7.40 (d, 1H), 7.23-7.12 (m, 3H), 6.95-6.92 (m, 2H), 6.86-6.82 (m, 2H), 6.80-6.77 (m, 1H), 5.14 (s, 2H), 4.34-4.25 (q, 4H), 3.98 (s, 2H), 3.74 (s, 3H), 3.67-3.63 (m, 2H), 3.40-3.35 (m, 4H), 3.33 (s, 3H), 3.10-3.07 (m, 2H), 2.94-2.90 (m, 4H), 2.74-2.67 (m, 1H), 2.65-2.59 (m, 2H), 2.57-2.51 (m, 3H), 2.28-2.24 (m, 1H), 2.06-2.02 (t, 2H), 1.89-1.83 (m, 2H), 1.56-1.52 (m, 2H), 1.47-1.44 (m, 2H), 1.29-1.21 (m, 18H), 1.01-0.97 (m, 1H), 0.58-0.45 (m, 7H), 0.33-0.23 (m, 2H), 0.12-0.10 (m, 1H).

### Example 156

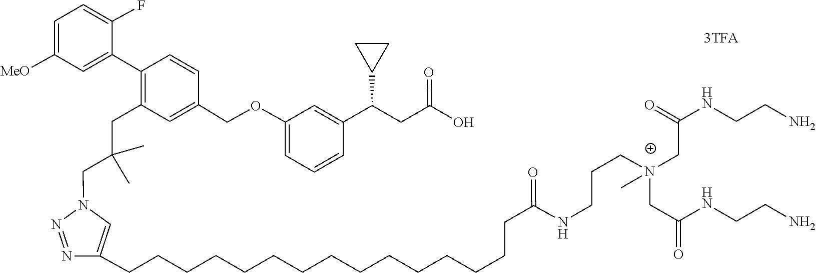

MS (ESI) m/z 541.8 [(M+H)/2]⁺. ¹H NMR (400 MHZ, DMSO-d₆): δ 8.95 (t, NH, 2H), 8.06-7.94 (br, 8H), 7.62 (s, 1H), 7.43 (s, 1H), 7.41-7.39 (d, 1H), 7.23-7.11 (m, 3H), 6.94-6.91 (m, 2H), 6.86-6.81 (m, 2H), 6.79-6.77 (m, 1H), 5.13 (s, 2H), 4.33-4.25 (q, 4H), 3.97 (s, 2H), 3.74 (s, 3H), 3.66-3.62 (m, 2H), 3.39-3.34 (m, 4H), 3.32 (s, 3H), 3.10-3.05 (m, 2H), 2.93-2.89 (m, 4H), 2.74-2.68 (m, 1H), 2.67-2.63 (m, 2H), 2.60-2.53 (m, 3H), 2.28-2.21 (m, 1H), 2.06-2.02 (t, 2H), 1.87-1.83 (m, 2H), 1.56-1.52 (m, 2H), 1.50-1.47 (m, 2H), 1.29-1.21 (m, 22H), 1.01-0.97 (m, 1H), 0.58-0.45 (m, 7H), 0.33-0.23 (m, 2H), 0.12-0.10 (m, 1H).

### Example 157

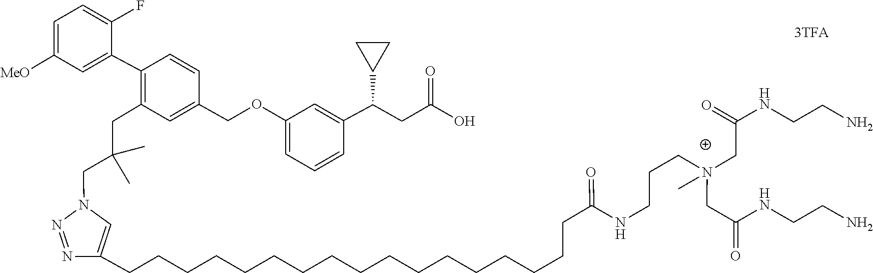

MS (ESI) m/z 555.8 [(M+H)/2]⁺. ¹H NMR (400 MHZ, DMSO-d₆): δ 8.95 (t, NH, 2H), 8.06-7.94 (br, 8H), 7.62 (s, 1H), 7.43 (s, 1H), 7.41-7.39 (d, 1H), 7.23-7.12 (m, 3H), 6.95-6.91 (m, 2H), 6.86-6.81 (m, 2H), 6.79-6.77 (m, 1H), 5.13 (s, 2H), 4.33-4.25 (q, 4H), 3.97 (s, 2H), 3.73 (s, 3H), 3.66-3.62 (m, 2H), 3.39-3.34 (m, 4H), 3.32 (s, 3H), 3.10-3.06 (m, 2H), 2.93-2.89 (m, 4H), 2.72-2.67 (m, 1H), 2.68-2.60 (m, 2H), 2.58-2.52 (m, 3H), 2.28-2.22 (m, 1H), 2.06-2.02 (t, 2H), 1.87-1.83 (m, 2H), 1.56-1.52 (m, 2H), 1.50-1.47 (m, 2H), 1.29-1.21 (m, 26H), 1.01-0.97 (m, 1H), 0.58-0.45 (m, 7H), 0.33-0.23 (m, 2H), 0.12-0.10 (m, 1H).

### Example 158

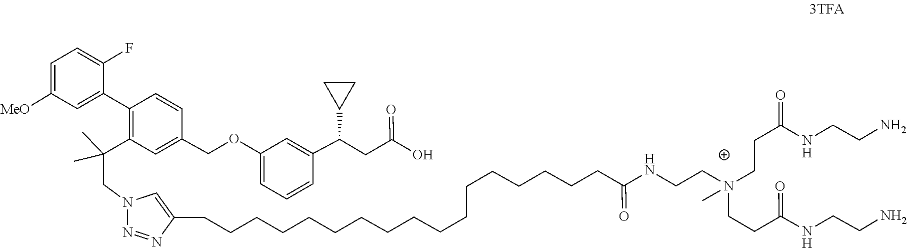

MS (ESI) m/z 1111.4 [M]⁺. ¹H NMR (400 MHZ, DMSO-d₆): δ 8.95 (t, NH, 2H), 8.40 (t, NH, 2H), 8.17 (t, NH, 1H), 7.91 (br, 6H), 7.55 (s, 1H), 7.38-7.36 (d, 1H), 7.22-7.15 (m, 2H), 7.12-7.11 (d, 1H), 6.99-6.95 (m, 1H), 6.93 (s, 1H), 6.86-6.84 (m, 2H), 6.73-6.71 (m, 1H), 5.09 (s, 2H), 4.43 (s, 2H), 3.76 (s, 3H), 3.58-3.55 (m, 4H), 3.46-3.44 (m, 2H), 3.34-3.29 (m, 6H), 3.03 (s, 3H), 2.90-2.86 (m, 4H), 2.70-2.58 (m, 6H), 2.50-2.47 (t, 2H), 2.29-2.25 (m, 1H), 2.10-2.07 (t, 2H), 1.51-1.43 (m, 4H), 1.25-1.13 (m, 29H), 1.06 (s, 3H), 1.05-1.00 (m, 1H), 0.51-0.48 (m, 1H), 0.31-0.17 (m, 2H), 0.13-0.09 (m, 1H).

### Example 159

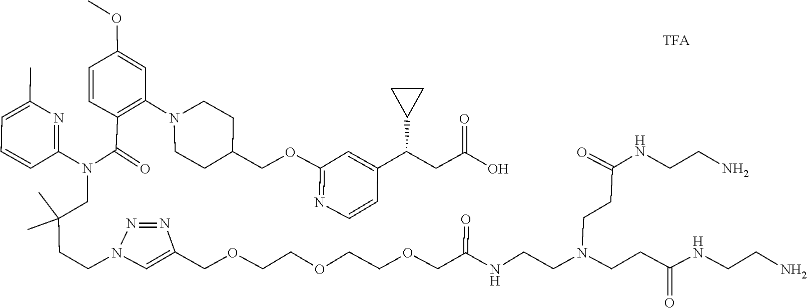 [SMILES: COc1ccc(C(=O)N(CC(C)(C)CCn2cc(COCCOCCOCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

MS (ESI) m/z 1142.4 [M+H]⁺. ¹H NMR (400 MHZ, DMSO-d₆): δ 8.41 (t, NH, 2H), 8.07-8.03 (m, 2H), 7.99 (s, 1H), 7.86 (br, NH₃, 6H), 7.30-7.26 (br, 1H), 7.17-6.15 (d, 1H), 6.93-6.92 (d, 1H), 6.86-6.84 (d, 1H), 6.72 (s, 1H), 6.54-6.52 (d, 1H), 6.45 (br, 1H), 6.21 (s, 1H), 4.51 (s, 2H), 4.35-4.30 (t, 2H), 4.20 (br, 2H), 4.13-4.12 (d, 2H), 3.92 (s, 2H), 3.69 (s, 3H), 3.58-3.55 (m, 8H), 3.52-3.47 (m, 2H), 3.41-3.37 (m, 4H), 3.33-3.28 (m, 4H), 3.24-3.21 (br, 2H), 2.90-2.86 (m, 4H), 2.69-2.67 (d, 2H), 2.64-2.61 (m, 4H), 2.36 (s, 3H), 2.27-2.21 (m, 1H), 1.76-1.65 (m, 5H), 1.37-1.25 (m, 2H), 1.02-0.99 (m, 1H), 0.78 (s, 6H), 0.52-0.49 (m, 1H), 0.36-0.25 (m, 2H), 0.17-0.13 (m, 1H).

### Example 160

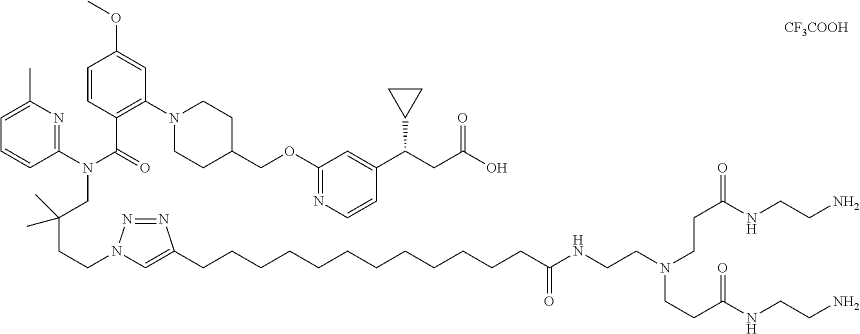 [SMILES: COc1ccc(C(=O)N(CC(C)(C)CCn2cc(CCCCCCCCCCCCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1 ; "CF3COOH"]

MS (ESI) m/z 1186.3 [M+H]⁺. ¹H NMR (400 MHZ, DMSO-d₆): δ 8.42 (t, NH, 2H), 8.07-8.04 (m, 2H), 7.97 (s, 1H), 7.86 (br, NH₃, 6H), 7.30-7.26 (br, 1H), 7.17-6.15 (d, 1H), 6.93-6.91 (d, 1H), 6.86-6.84 (d, 1H), 6.72 (s, 1H), 6.54-6.52 (d, 1H), 6.45 (br, 1H), 6.21 (s, 1H), 4.50 (s, 2H), 4.40-4.35 (t, 2H), 4.20 (br, 2H), 4.13-4.12 (d, 2H), 3.92 (s, 2H), 3.69 (s, 3H), 3.58-3.47 (m, 14H), 3.41-3.37 (m, 4H), 3.33-3.28 (m, 4H), 3.23-3.21 (br, 2H), 2.90-2.86 (m, 4H), 2.69-2.67 (d, 2H), 2.64-2.61 (m, 4H), 2.36 (s, 3H), 2.26-2.20 (m, 1H), 1.76-1.65 (m, 5H), 1.37-1.25 (m, 2H), 1.02-0.99 (m, 1H), 0.78 (s, 6H), 0.52-0.49 (m, 1H), 0.36-0.25 (m, 2H), 0.17-0.13 (m, 1H).

### Example 161

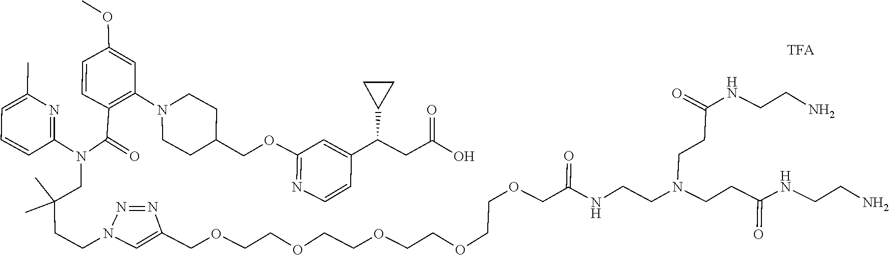 [SMILES: COc1ccc(C(=O)N(CC(C)(C)CCn2cc(COCCOCCOCCOCCOCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

MS (ESI) m/z 1230.3 [M+H]⁺. ¹H NMR (400 MHZ, DMSO-d₆): δ 8.40 (t, NH, 2H), 8.07-8.04 (m, 2H), 7.97 (s, 1H), 7.86 (br, NH₃, 6H), 7.28-7.26 (br, 1H), 7.17-6.15 (d, 1H), 6.93-6.91 (d, 1H), 6.86-6.84 (d, 1H), 6.72 (s, 1H), 6.54-6.52 (d, 1H), 6.45 (br, 1H), 6.21 (s, 1H), 4.50 (s, 2H), 4.40-4.35 (t, 2H), 4.20 (br, 2H), 4.13-4.11 (d, 2H), 3.92 (s, 2H), 3.69 (s, 3H), 3.58-3.51 (m, 18H), 3.41-3.37 (m, 4H), 3.33-3.28 (m, 4H), 3.23-3.21 (br, 2H), 2.90-2.86 (m, 4H), 2.69-2.67 (d, 2H), 2.64-2.61 (m, 4H), 2.36 (s, 3H), 2.26-2.20 (m, 1H), 1.72-1.65 (m, 5H), 1.37-1.25 (m, 2H), 1.02-0.99 (m, 1H), 0.78 (s, 6H), 0.52-0.49 (m, 1H), 0.36-0.25 (m, 2H), 0.17-0.13 (m, 1H).

### Example 162

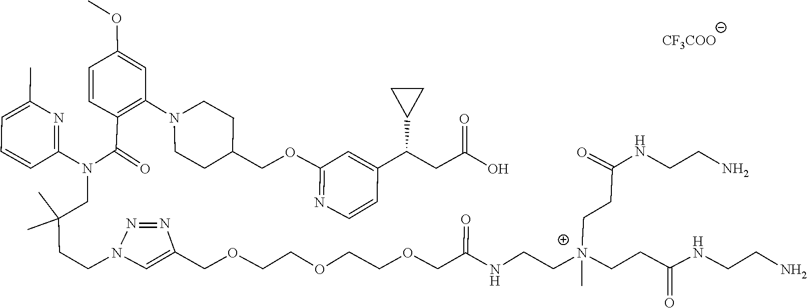 [SMILES: COc1ccc(C(=O)N(CC(C)(C)CCn2cc(COCCOCCOCC(=O)NCC[N+](C)(CCC(=O)NCCN)CCC(=O)NCCN)nn2)c2cccc(C)n2)c(N2CCC(COc3cc([C@@H](CC(=O)O)C4CC4)ccn3)CC2)c1 ; "CF3COO"]

MS (ESI) m/z 1156.3 [M]⁺. ¹H NMR (400 MHZ, DMSO-d₆): δ 8.36 (t, NH, 2H), 8.09-8.03 (t, 2H), 7.99 (s, 1H), 7.87 (br, NH₃, 6H), 7.29-7.26 (br, 1H), 7.17-7.15 (d, 1H), 6.92-6.91 (d, 1H), 6.86-6.84 (d, 1H), 6.71 (s, 1H), 6.54-6.51 (d, 1H), 6.45 (br, 1H), 6.20 (s, 1H), 4.51 (s, 2H), 4.40-4.35 (t, 2H), 4.20 (br, 2H), 4.13-4.11 (d, 2H), 3.92 (s, 2H), 3.69 (s, 3H), 3.60-3.52 (m, 14H), 3.35-3.29 (m, 6H), 3.04 (s, 3H), 2.89-2.85 (m, 4H), 2.69-2.65 (m, 6H), 2.36 (s, 3H), 2.26-2.20 (m, 1H), 1.73-1.65 (m, 5H), 1.37-1.25 (m, 2H), 1.02-0.98 (m, 1H), 0.78 (s, 6H), 0.52-0.49 (m, 1H), 0.33-0.24 (m, 2H), 0.17-0.13 (m, 1H).

### Example 163

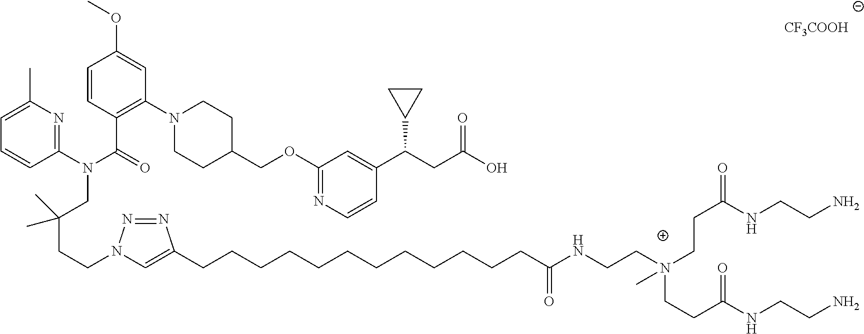

MS (ESI) m/z 1200.3 [M]⁺. ¹H NMR (400 MHZ, DMSO-d₆): δ 8.35 (t, NH, 2H), 8.07-8.03 (t, 2H), 7.98 (s, 1H), 7.86 (br, NH₃, 6H), 7.29-7.26 (br, 1H), 7.17-7.15 (d, 1H), 6.92-6.91 (d, 1H), 6.86-6.84 (d, 1H), 6.71 (s, 1H), 6.54-6.51 (d, 1H), 6.45 (br, 1H), 6.21 (s, 1H), 4.50 (s, 2H), 4.39 (br, 2H), 4.20 (br, 2H), 4.13-4.11 (d, 2H), 3.92 (s, 2H), 3.69 (s, 3H), 3.58-3.52 (m, 18H), 3.35-3.29 (m, 6H), 3.04 (s, 3H), 2.89-2.85 (m, 4H), 2.69-2.65 (m, 6H), 2.36 (s, 3H), 2.26-2.20 (m, 1H), 1.73-1.65 (m, 5H), 1.37-1.25 (m, 2H), 1.02-0.98 (m, 1H), 0.78 (s, 6H), 0.52-0.49 (m, 1H), 0.33-0.24 (m, 2H), 0.17-0.13 (m, 1H).

### Example 164

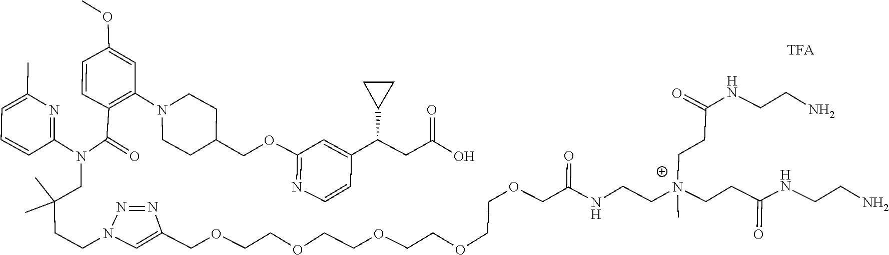 [SMILES: COc1ccc(C(=O)N(CC(C)(C)CCn2cc(COCCOCCOCCOCCOCC(=O)NCC[N+](C)(CCC(=O)NCCN)CCC(=O)NCCN)nn2)c2cccc(C)n2)c(N2CCC(COc3cc([C@@H](CC(=O)O)C4CC4)ccn3)CC2)c1]

MS (ESI) m/z 1244.3 [M]⁺. ¹H NMR (400 MHZ, DMSO-d₆): δ 8.35 (t, NH, 2H), 8.07-8.03 (t, 2H), 7.97 (s, 1H), 7.87 (br, NH₃, 6H), 7.29-7.26 (br, 1H), 7.17-7.15 (d, 1H), 6.92-6.91 (d, 1H), 6.86-6.84 (d, 1H), 6.71 (s, 1H), 6.54-6.51 (d, 1H), 6.45 (br, 1H), 6.21 (s, 1H), 4.50 (s, 2H), 4.39 (br, 2H), 4.20 (br, 2H), 4.13-4.11 (d, 2H), 3.93 (s, 2H), 3.69 (s, 3H), 3.58-3.52 (m, 18H), 3.35-3.29 (m, 6H), 3.04 (s, 3H), 2.89-2.85 (m, 4H), 2.69-2.65 (m, 6H), 2.36 (s, 3H), 2.26-2.20 (m, 1H), 1.75-1.65 (m, 5H), 1.37-1.25 (m, 2H), 1.02-0.98 (m, 1H), 0.78 (s, 6H), 0.52-0.49 (m, 1H), 0.33-0.24 (m, 2H), 0.17-0.13 (m, 1H).

### Example 165

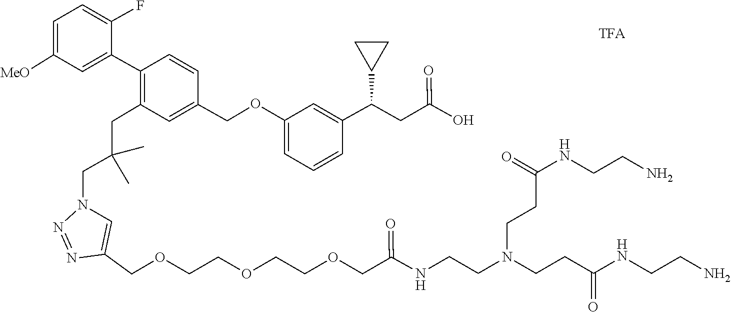 [SMILES: COc1ccc(F)c(-c2ccc(COc3cccc([C@@H](CC(=O)O)C4CC4)c3)cc2CC(C)(C)Cn2cc(COCCOCCOCC(=O)NCCN(CCC(=O)NCCN)CCC(=O)NCCN)nn2)c1]

MS (ESI) m/z 1004.3 [M+H]⁺. ¹H NMR (400 MHZ, DMSO-d₆): δ 8.42 (t, NH, 2H), 8.06 (t, NH, 1H), 7.90 (br, 6H), 7.91 (s, 1H), 7.44 (s, 1H), 7.42-7.40 (d, 1H), 7.24-7.12 (m, 3H), 6.95-6.91 (m, 2H), 6.86-6.82 (m, 2H), 6.79-6.77 (m, 1H), 5.14 (s, 2H), 4.49 (s, 2H), 4.10 (s, 2H), 3.91 (s, 2H), 3.73 (s, 3H), 3.59-3.55 (m, 8H), 3.51-3.48 (m, 2H), 3.40-3.36 (m, 4H), 3.33-3.29 (m, 4H), 3.24-3.21 (m, 2H), 2.89-2.87 (m, 4H), 2.74-2.53 (m, 8H), 2.28-2.24 (m, 1H), 1.02-0.97 (m, 1H), 0.60-0.45 (br, 7H), 0.33-0.20 (m, 2H), 0.12-0.09 (m, 1H).

### Example 166

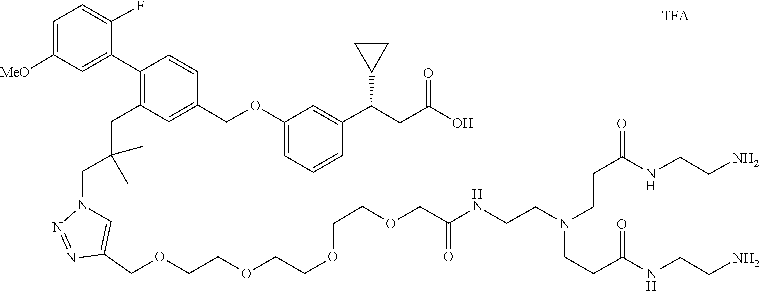 [SMILES: COc1ccc(F)c(-c2ccc(COc3cccc([C@@H](CC(=O)O)C4CC4)c3)cc2CC(C)(C)Cn2cc(COCCOCCOCCOCC(=O)NCCN(CCC(=O)NCCN)CCC(=O)NCCN)nn2)c1]

MS (ESI) m/z 1048.2 [M+H]⁺. ¹H NMR (400 MHZ, DMSO-d₆): δ 8.45 (t, NH, 2H), 8.07 (t, NH, 1H), 7.95 (br, 6H), 7.90 (s, 1H), 7.44 (s, 1H), 7.42-7.40 (d, 1H), 7.24-7.13 (m, 3H), 6.96-6.92 (m, 2H), 6.87-6.82 (m, 2H), 6.79-6.77 (m, 1H), 5.14 (s, 2H), 4.49 (s, 2H), 4.05 (s, 2H), 3.92 (s, 2H), 3.74 (s, 3H), 3.59-3.49 (m, 14H), 3.41-3.38 (m, 4H), 3.34-3.24 (m, 4H), 3.24-3.21 (m, 2H), 2.90-2.87 (m, 4H), 2.74-2.53 (m, 8H), 2.28-2.24 (m, 1H), 1.02-0.97 (m, 1H), 0.60-0.45 (br, 7H), 0.31-0.20 (m, 2H), 0.12-0.09 (m, 1H).

### Example 167

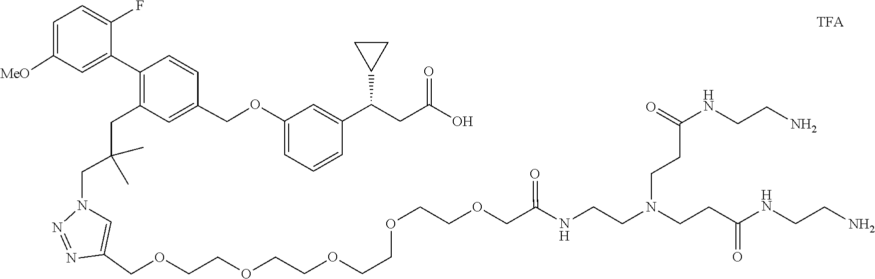 [SMILES: COc1ccc(F)c(-c2ccc(COc3cccc([C@@H](CC(=O)O)C4CC4)c3)cc2CC(C)(C)Cn2cc(COCCOCCOCCOCCOCC(=O)NCCN(CCC(=O)NCCN)CCC(=O)NCCN)nn2)c1]

MS (ESI) m/z 1092.3 [M+H]⁺. ¹H NMR (400 MHZ, DMSO-d₆): δ 8.41 (t, NH, 2H), 8.06 (t, NH, 1H), 7.88 (br, 6H), 7.90 (s, 1H), 7.44 (s, 1H), 7.42-7.40 (d, 1H), 7.24-7.13 (m, 3H), 6.96-6.91 (m, 2H), 6.87-6.82 (m, 2H), 6.79-6.77 (m, 1H), 5.14 (s, 2H), 4.49 (s, 2H), 4.03 (s, 2H), 3.92 (s, 2H), 3.74 (s, 3H), 3.60-3.49 (m, 18H), 3.40-3.38 (m, 4H), 3.34-3.29 (m, 4H), 3.23-3.19 (m, 2H), 2.90-2.87 (m, 4H), 2.74-2.57 (m, 8H), 2.27-2.24 (m, 1H), 1.01-0.97 (m, 1H), 0.57-0.46 (br, 7H), 0.31-0.20 (m, 2H), 0.12-0.09 (m, 1H).

### Example 168

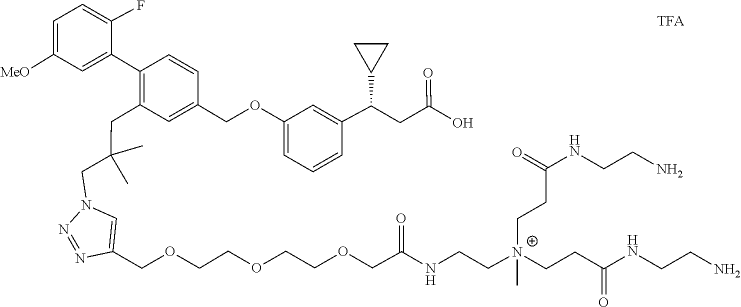 [SMILES: COc1ccc(F)c(-c2ccc(COc3cccc([C@@H](CC(=O)O)C4CC4)c3)cc2CC(C)(C)Cn2cc(COCCOCCOCC(=O)NCC[N+](C)(CCC(=O)NCCN)CCC(=O)NCCN)nn2)c1]

MS (ESI) m/z 1019.3 [M+H]⁺. ¹H NMR (400 MHz, DMSO-d₆): δ 8.38 (t, NH, 2H), 8.08 (t, NH, 1H), 7.92 (br, 6H), 7.91 (s, 1H), 7.44 (s, 1H), 7.42-7.40 (d, 1H), 7.24-7.12 (m, 3H), 6.95-6.91 (m, 2H), 6.86-6.82 (m, 2H), 6.79-6.77 (m, 1H), 5.14 (s, 2H), 4.49 (s, 2H), 4.04 (s, 2H), 3.92 (s, 2H), 3.73 (s, 3H), 3.63-3.55 (m, 14H), 3.35-3.29 (m, 6H), 3.04 (s, 3H), 2.90-2.86 (m, 4H), 2.74-2.57 (m, 8H), 2.28-2.22 (m, 1H), 1.01-0.95 (m, 1H), 0.57-0.46 (br, 7H), 0.31-0.20 (m, 2H), 0.12-0.09 (m, 1H).

### Example 169

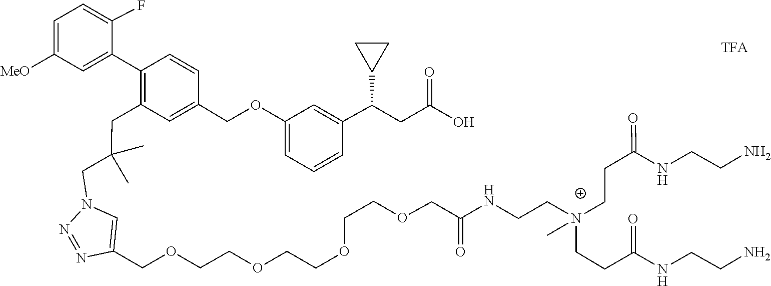

MS (ESI) m/z 1062.3 [M+H]⁺. ¹H NMR (400 MHZ, DMSO-d₆): δ 8.39 (t, NH, 2H), 8.08 (t, NH, 1H), 7.92 (br, 6H), 7.90 (s, 1H), 7.44 (s, 1H), 7.42-7.40 (d, 1H), 7.24-7.12 (m, 3H), 6.96-6.91 (m, 2H), 6.86-6.82 (m, 2H), 6.79-6.77 (m, 1H), 5.14 (s, 2H), 4.49 (s, 2H), 4.04 (s, 2H), 3.92 (s, 2H), 3.74 (s, 3H), 3.63-3.55 (m, 18H), 3.36-3.29 (m, 6H), 3.04 (s, 3H), 2.90-2.86 (m, 4H), 2.74-2.53 (m, 8H), 2.28-2.21 (m, 1H), 1.01-0.97 (m, 1H), 0.58-0.46 (br, 7H), 0.30-0.20 (m, 2H), 0.12-0.09 (m, 1H).

### Example 170

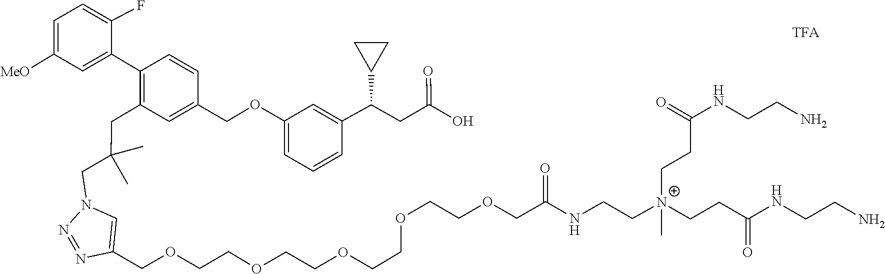 [SMILES: COc1ccc(F)c(-c2ccc(COc3cccc([C@@H](CC(=O)O)C4CC4)c3)cc2CC(C)(C)Cn2cc(COCCOCCOCCOCCOCC(=O)NCC[N+](C)(CCC(=O)NCCN)CCC(=O)NCCN)nn2)c1]

MS (ESI) m/z 1106.3 [M+H]⁺. ¹H NMR (400 MHZ, DMSO-d₆): δ 8.39 (t, NH, 2H), 8.09 (t, NH, 1H), 7.92 (br, 6H), 7.90 (s, 1H), 7.44 (s, 1H), 7.42-7.40 (d, 1H), 7.24-7.12 (m, 3H), 6.96-6.91 (m, 2H), 6.87-6.82 (m, 2H), 6.79-6.77 (m, 1H), 5.14 (s, 2H), 4.49 (s, 2H), 4.03 (s, 2H), 3.93 (s, 2H), 3.74 (s, 3H), 3.61-3.51 (m, 22H), 3.36-3.30 (m, 6H), 3.04 (s, 3H), 2.90-2.86 (m, 4H), 2.74-2.51 (m, 8H), 2.28-2.22 (m, 1H), 1.01-0.97 (m, 1H), 0.58-0.46 (br, 7H), 0.31-0.21 (m, 2H), 0.12-0.09 (m, 1H).

### Example 171

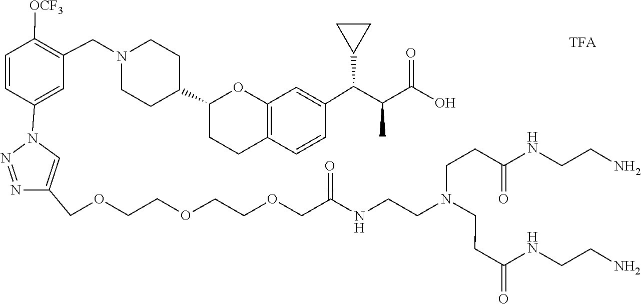 [SMILES: C[C@H](C(=O)O)[C@H](c1ccc2c(c1)O[C@@H](C1CCN(Cc3cc(-n4cc(COCCOCCOCC(=O)NCCN(CCC(=O)NCCN)CCC(=O)NCCN)nn4)ccc3OC(F)(F)F)CC1)CC2)C1CC1]

MS (ESI) m/z 1031.1 [M+H]⁺. ¹H NMR (400 MHZ, DMSO-d₆): δ 8.86 (s, 1H), 8.41 (br, 3H), 8.14-8.12 (d, 1H), 8.07 (t, NH, 1H), 7.89 (br, 6H), 7.76-7.74 (d, 1H), 6.97-6.95 (d, 1H), 6.66-6.64 (d, 1H), 6.51 (s, 1H), 4.66 (s, 2H), 4.47 (s, 2H), 3.92 (s, 2H), 3.82-3.80 (m, 1H), 3.66-3.59 (m, 8H), 3.50-3.45 (m, 2H), 3.40-3.35 (m, 6H), 3.33-3.29 (m, 4H), 3.20-3.14 (m, 4H), 2.90-2.86 (m, 4H), 2.77-2.62 (m, 7H), 2.10-2.06 (m, 1H), 1.96-1.81 (m, 4H), 1.69-1.60 (m, 3H), 1.09-1.04 (m, 1H), 0.82-0.80 (d, 3H), 0.54-0.50 (m, 1H), 0.30-0.19 (m, 2H), −0.05 ˜−0.10 (m, 1H).

### Example 172

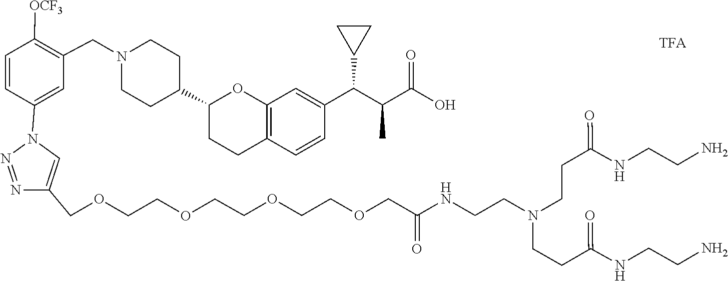 [SMILES: C[C@H](C(=O)O)[C@H](c1ccc2c(c1)O[C@@H](C1CCN(Cc3cc(-n4cc(COCCOCCOCCOCC(=O)NCCN(CCC(=O)NCCN)CCC(=O)NCCN)nn4)ccc3OC(F)(F)F)CC1)CC2)C1CC1]

MS (ESI) m/z 1075.3 [M+H]⁺. ¹H NMR (400 MHZ, DMSO-d₆): δ 8.41-8.40 (br, 3H), 8.15-8.12 (d, 1H), 8.06 (t, NH, 1H), 7.88 (br, 6H), 7.75-7.74 (d, 1H), 6.97-6.95 (d, 1H), 6.66-6.64 (d, 1H), 6.51 (s, 1H), 4.65 (s, 2H), 4.47 (s, 2H), 3.92 (s, 2H), 3.83-3.80 (m, 1H), 3.64-3.54 (m, 12H), 3.50-3.45 (m, 2H), 3.40-3.35 (m, 6H), 3.33-3.28 (m, 4H), 3.20-3.14 (m, 4H), 2.90-2.86 (m, 4H), 2.77-2.60 (m, 7H), 2.10-2.06 (m, 1H), 1.96-1.81 (m, 4H), 1.69-1.60 (m, 3H), 1.09-1.04 (m, 1H), 0.82-0.80 (d, 3H), 0.52-0.48 (m, 1H), 0.28-0.19 (m, 2H), −0.05 ˜−0.10 (m, 1H).

### Example 173

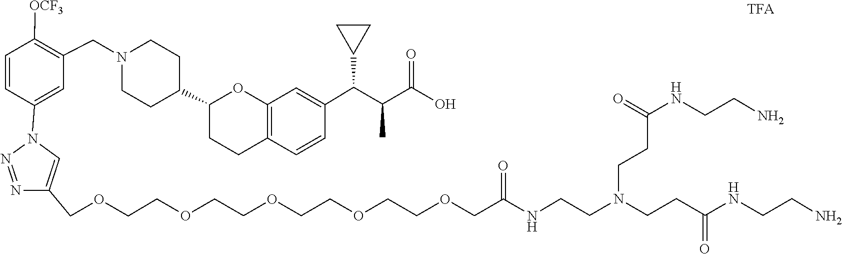 [SMILES: C[C@H](C(=O)O)[C@H](c1ccc2c(c1)O[C@@H](C1CCN(Cc3cc(-n4cc(COCCOCCOCCOCCOCC(=O)NCCN(CCC(=O)NCCN)CCC(=O)NCCN)nn4)ccc3OC(F)(F)F)CC1)CC2)C1CC1]

MS (ESI) m/z 1118.6 [M+H]⁺. ¹H NMR (400 MHZ, DMSO-d₆): δ 8.41-8.40 (br, 3H), 8.14-8.12 (d, 1H), 8.05 (t, NH, 1H), 7.88 (br, 6H), 7.75-7.73 (d, 1H), 6.97-6.95 (d, 1H), 6.66-6.64 (d, 1H), 6.51 (s, 1H), 4.65 (s, 2H), 4.47 (s, 2H), 3.91 (s, 2H), 3.82-3.80 (m, 1H), 3.64-3.52 (m, 16H), 3.50-3.45 (m, 2H), 3.40-3.35 (m, 6H), 3.33-3.28 (m, 4H), 3.20-3.14 (m, 4H), 2.90-2.86 (m, 4H), 2.77-2.62 (m, 7H), 2.08-2.05 (m, 1H), 1.96-1.81 (m, 4H), 1.66-1.60 (m, 3H), 1.06-1.02 (m, 1H), 0.82-0.80 (d, 3H), 0.52-0.48 (m, 1H), 0.28-0.19 (m, 2H), −0.05 ˜−0.10 (m, 1H).

### Example 174

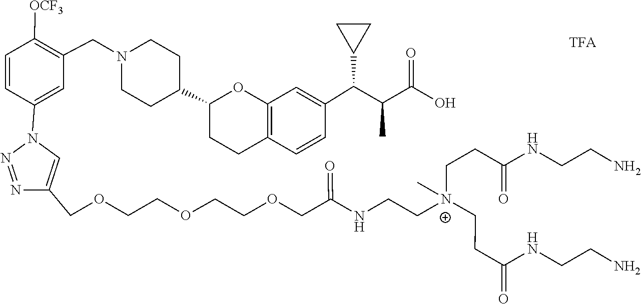 [SMILES: C[C@H](C(=O)O)[C@H](c1ccc2c(c1)O[C@@H](C1CCN(Cc3cc(-n4cc(COCCOCCOCC(=O)NCC[N+](C)(CCC(=O)NCCN)CCC(=O)NCCN)nn4)ccc3OC(F)(F)F)CC1)CC2)C1CC1]

MS (ESI) m/z 1045.3 [M]⁺. ¹H NMR (400 MHZ, DMSO-d₆): δ 8.85 (s, 1H), 8.40-8.37 (br, 3H), 8.14-8.08 (m, 2H), 7.90 (br, 6H), 7.75-7.73 (d, 1H), 6.97-6.95 (d, 1H), 6.66-6.64 (d, 1H), 6.51 (s, 1H), 4.66 (s, 2H), 4.48 (s, 2H), 3.93 (s, 2H), 3.81-3.79 (m, 1H), 3.65-3.59 (m, 8H), 3.60-3.55 (m, 4H), 3.55-3.50 (m, 2H), 3.32-3.29 (m, 8H), 3.14 (br, 2H), 3.04 (s, 3H), 2.90-2.86 (m, 4H), 2.77-2.58 (m, 7H), 2.08-2.05 (m, 1H), 1.96-1.81 (m, 4H), 1.66-1.60 (m, 3H), 1.06-1.02 (m, 1H), 0.82-0.80 (d, 3H), 0.52-0.49 (m, 1H), 0.28-0.19 (m, 2H), −0.05 ˜−0.10 (m, 1H).

### Example 175

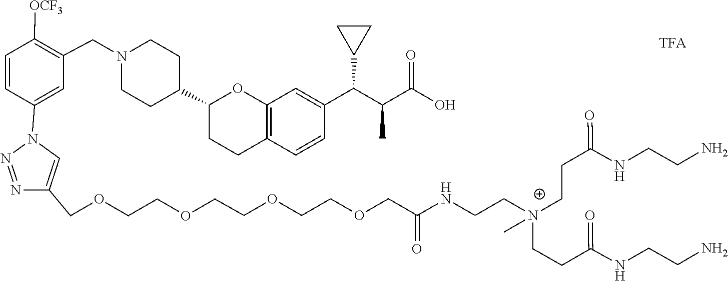 [SMILES: C[C@H](C(=O)O)[C@H](c1ccc2c(c1)O[C@@H](C1CCN(Cc3cc(-n4cc(COCCOCCOCCOCC(=O)NCC[N+](C)(CCC(=O)NCCN)CCC(=O)NCCN)nn4)ccc3OC(F)(F)F)CC1)CC2)C1CC1]

MS (ESI) m/z 1090.2 [M]⁺. ¹H NMR (400 MHZ, DMSO-d₆): δ 8.86 (s, 1H), 8.43-8.38 (br, 1H), 8.36-8.31 (t, 2H), 8.15-8.12 (br, 1H), 8.07 (t, NH, 1H), 7.83 (br, 6H), 7.78-7.76 (d, 1H), 6.97-6.95 (d, 1H), 6.66-6.64 (d, 1H), 6.51 (s, 1H), 4.65 (s, 2H), 4.48 (br, 2H), 3.93 (s, 2H), 3.82-3.80 (m, 1H), 3.65-3.52 (m, 12H), 3.60-3.55 (m, 4H), 3.55-3.50 (m, 2H), 3.32-3.29 (m, 8H), 3.14 (br, 2H), 3.04 (s, 3H), 2.90-2.86 (m, 4H), 2.77-2.59 (m, 7H), 2.10-2.09 (m, 1H), 1.96-1.81 (m, 4H), 1.66-1.60 (m, 3H), 1.05-1.00 (m, 1H), 0.82-0.80 (d, 3H), 0.52-0.49 (m, 1H), 0.28-0.19 (m, 2H), −0.05 ˜−0.10 (m, 1H).

### Example 176

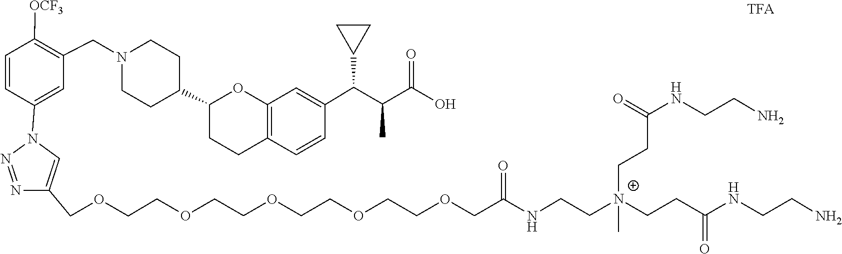 [SMILES: C[C@H](C(=O)O)[C@H](c1ccc2c(c1)O[C@@H](C1CCN(Cc3cc(-n4cc(COCCOCCOCCOCCOCC(=O)NCC[N+](C)(CCC(=O)NCCN)CCC(=O)NCCN)nn4)ccc3OC(F)(F)F)CC1)CC2)C1CC1]

MS (ESI) m/z 1133.3 [M]⁺. ¹H NMR (400 MHZ, DMSO-d₆): δ 8.84 (s, 1H), 8.40-8.37 (m, 3H), 8.14-8.12 (d, 1H), 8.09 (t, NH, 1H), 7.90 (br, 6H), 7.75-7.73 (d, 1H), 6.97-6.95 (d, 1H), 6.66-6.64 (d, 1H), 6.51 (s, 1H), 4.65 (s, 2H), 4.47 (br, 2H), 3.92 (s, 2H), 3.82-3.80 (m, 1H), 3.64-3.52 (m, 22H), 3.32-3.29 (m, 8H), 3.14 (br, 2H), 3.04 (s, 3H), 2.88-2.86 (m, 4H), 2.77-2.60 (m, 7H), 2.10-2.09 (m, 1H), 1.96-1.81 (m, 4H), 1.66-1.60 (m, 3H), 1.05-1.00 (m, 1H), 0.82-0.80 (d, 3H), 0.52-0.49 (m, 1H), 0.28-0.19 (m, 2H), −0.05 ˜−0.10 (m, 1H).

### Example 177

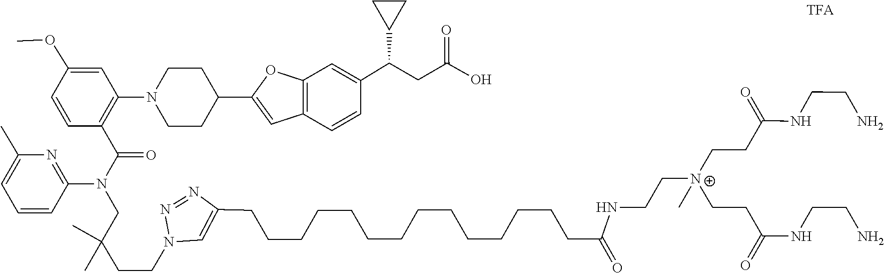 [SMILES: COc1ccc(C(=O)N(CC(C)(C)CCn2cc(CCCCCCCCCCCCCCC(=O)NCC[N+](C)(CCC(=O)NCCN)CCC(=O)NCCN)nn2)c2cccc(C)n2)c(N2CCC(c3cc4ccc([C@@H](CC(=O)O)C5CC5)cc4o3)CC2)c1]

MS (ESI) m/z 1229.5 [M]⁺. ¹H NMR (400 MHZ, DMSO-d₆): δ 8.40 (t, NH, 2H), 8.18 (t, NH, 1H), 7.92 (br, NH₃, 6H), 7.68 (s, 1H), 7.48-7.46 (d, 1H), 7.41 (s, 1H), 7.31-7.27 (br, 1H), 7.19-7.17 (d, 1H), 7.13-7.11 (d, 1H), 6.88-6.86 (d, 1H), 6.57-6.55 (d, 1H), 6.54 (s, 1H), 6.44 (br, 1H), 6.26 (s, 1H), 4.32 (br, 2H), 4.20 (br, 2H), 3.69 (s, 3H), 3.60-3.56 (m, 4H), 3.47-3.44 (m, 2H), 3.34-3.30 (m, 6H), 3.04 (s, 3H), 2.90-2.86 (m, 4H), 2.78-2.63 (m, 10H), 2.57-2.53 (m, 2H), 2.37 (s, 3H), 2.41-2.35 (m, 1H), 2.11-2.07 (t, 2H), 1.96-1.93 (m, 3H), 1.69-1.65 (m, 2H), 1.56-1.45 (, m, 6H), 1.29-1.18 (m, 20H), 1.10-1.06 (m, 1H), 0.78 (s, 6H), 0.53-0.50 (m, 1H), 0.32-0.25 (m, 2H), 0.23-0.18 (m, 1H).

### Example 178

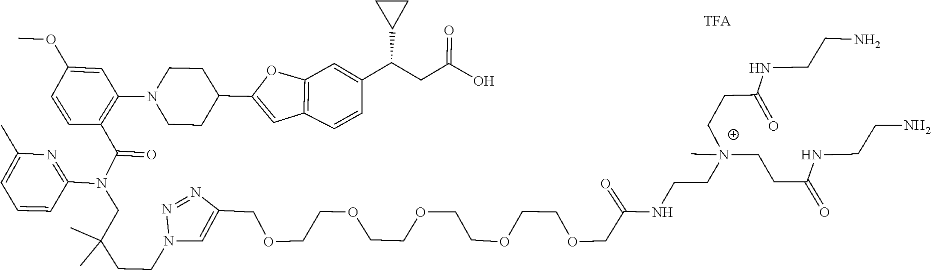 [SMILES: COc1ccc(C(=O)N(CC(C)(C)CCn2cc(COCCOCCOCCOCCOCC(=O)NCC[N+](C)(CCC(=O)NCCN)CCC(=O)NCCN)nn2)c2cccc(C)n2)c(N2CCC(c3cc4ccc([C@@H](CC(=O)O)C5CC5)cc4o3)CC2)c1]

MS (ESI) m/z 1253.3 [M]⁺. ¹H NMR (400 MHZ, DMSO-d₆): δ 8.37 (t, NH, 2H), 8.08 (t, NH, 1H), 7.97 (s, 1H), 7.88 (br, NH₃, 6H), 7.48-7.46 (d, 1H), 7.41 (s, 1H), 7.31-7.27 (br, 1H), 7.21-7.19 (d, 1H), 7.15-7.11 (d, 1H), 6.88-6.86 (d, 1H), 6.57-6.55 (d, 1H), 6.54 (s, 1H), 6.43 (br, 1H), 6.25 (s, 1H), 4.48 (s, 2H), 3.93 (s, 2H), 4.37 (br, 2H), 4.25 (br, 2H), 3.71 (s, 3H), 3.58-3.57 (m, 4H), 3.55-3.50 (m, 18H), 3.34-3.29 (m, 6H), 3.04 (s, 3H), 2.90-2.85 (m, 4H), 2.76-2.66 (m, 10H), 2.37 (s, 3H), 2.41-2.35 (m, 1H), 2.01-1.93 (m, 3H), 1.70-1.66 (m, 2H), 1.50-1.45 (, m, 2H), 1.10-1.06 (m, 1H), 0.78 (s, 6H), 0.53-0.50 (m, 1H), 0.32-0.25 (m, 2H), 0.23-0.18 (m, 1H).

### Example 179

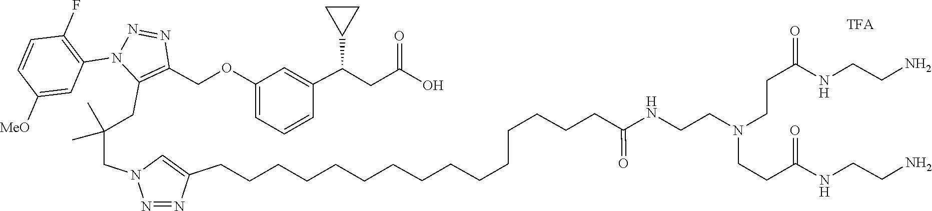 [SMILES: COc1ccc(F)c(-n2nnc(COc3cccc([C@@H](CC(=O)O)C4CC4)c3)c2CC(C)(C)Cn2cc(CCCCCCCCCCCCCCCC(=O)NCCN(CCC(=O)NCCN)CCC(=O)NCCN)nn2)c1]

MS (ESI) m/z 1073.5 [M+H]⁺. ¹H NMR (400 MHZ, DMSO-d₆): δ 8.40 (t, NH, 2H), 8.13 (t, NH, 1H), 7.88 (br, 6H), 7.66 (s, 1H), 7.45-7.40 (t, 1H), 7.26-7.16 (m, 3H), 6.92 (s, 1H), 6.91-6.86 (m, 2H), 5.17 (s, 2H), 4.13 (s, 2H), 3.78 (s, 3H), 3.41-336 (m, 6H), 3.33-3.28 (m, 4H), 3.18-3.15 (m, 2H), 2.89-2.85 (m, 4H), 2.70-2.60 (m, 7H), 2.54-2.50 (m, 3H), 2.30-2.25 (m, 1H), 2.10-2.06 (t, 2H), 1.52-1.44 (m, 4H), 1.30-1.20 (m, 22H), 1.02-0.98 (m, 1H), 0.59 (br, 6H), 0.52-0.48 (m, 1H), 0.32-0.23 (m, 2H), 0.13-0.10 (m, 1H).

### Example 180

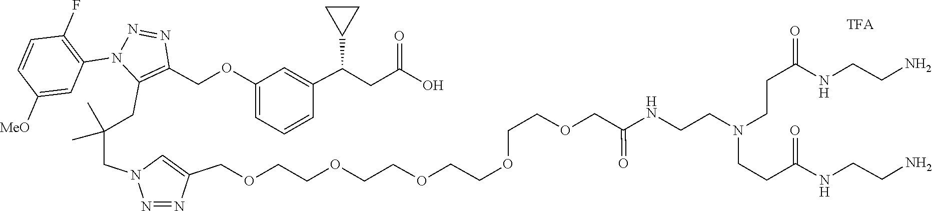 [SMILES: COc1ccc(F)c(-n2nnc(COc3cccc([C@@H](CC(=O)O)C4CC4)c3)c2CC(C)(C)Cn2cc(COCCOCCOCCOCCOCC(=O)NCCN(CCC(=O)NCCN)CCC(=O)NCCN)nn2)c1]

MS (ESI) m/z 1083.3 [M+H]⁺. ¹H NMR (400 MHz, DMSO-d₆): δ 8.41 (t, NH, 2H), 8.06 (t, NH, 1H), 7.93 (s, 1H), 7.88 (br, 6H), 7.46-7.41 (t, 1H), 7.26-7.17 (m, 3H), 6.93 (s, 1H), 6.91-6.87 (m, 2H), 5.18 (s, 2H), 4.47 (s, 2H), 4.18 (s, 2H), 3.92 (s, 2H), 3.78 (s, 3H), 3.68-3.50 (m, 18H), 3.40-3.37 (m, 4H), 3.33-3.28 (m, 4H), 3.22-3.19 (m, 2H), 2.89-2.86 (m, 4H), 2.71-2.60 (m, 7H), 2.52-2.49 (m, 1H), 2.30-2.24 (m, 1H), 1.03-0.97 (m, 1H), 0.61 (br, 6H), 0.53-0.47 (m, 1H), 0.33-0.25 (m, 2H), 0.15-0.10 (m, 1H).

### Example 181

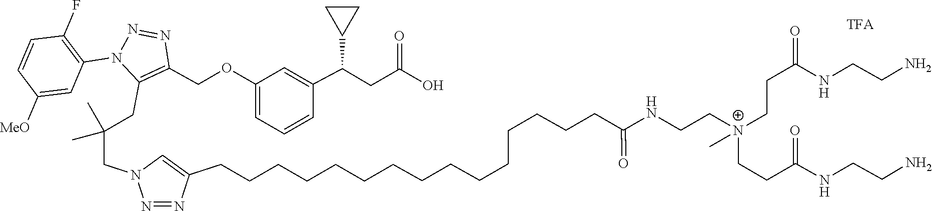 [SMILES: COc1ccc(F)c(-n2nnc(COc3cccc([C@@H](CC(=O)O)C4CC4)c3)c2CC(C)(C)Cn2cc(CCCCCCCCCCCCCCCC(=O)NCC[N+](C)(CCC(=O)NCCN)CCC(=O)NCCN)nn2)c1]

MS (ESI) m/z 1087.5 [M]⁺. ¹H NMR (400 MHZ, DMSO-d₆): δ 8.37 (t, NH, 2H), 8.15 (t, NH, 1H), 7.88 (br, 6H), 7.66 (s, 1H), 7.45-7.40 (t, 1H), 7.27-7.15 (m, 3H), 6.92 (s, 1H), 6.90-6.86 (m, 2H), 5.17 (s, 2H), 4.13 (s, 2H), 3.78 (s, 3H), 3.56-3.50 (m, 4H), 3.47-3.42 (m, 2H), 3.33-3.29 (m, 6H), 3.03 (s, 3H), 2.89-2.86 (m, 4H), 2.69-2.59 (m, 7H), 2.54-2.50 (m, 3H), 2.30-2.25 (m, 1H), 2.10-2.06 (t, 2H), 1.53-1.46 (m, 4H), 1.30-1.22 (m, 22H), 1.02-0.98 (m, 1H), 0.59 (br, 6H), 0.52-0.48 (m, 1H), 0.32-0.23 (m, 2H), 0.13-0.10 (m, 1H).

### Example 182

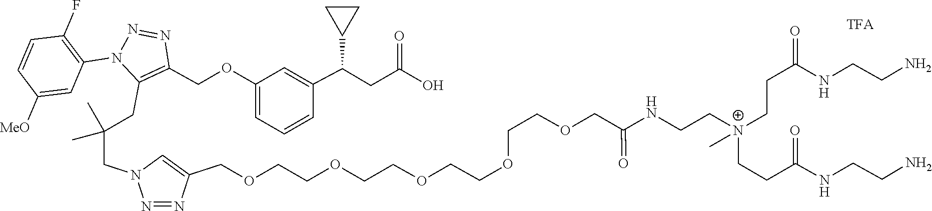 [SMILES: COc1ccc(F)c(-n2nnc(COc3cccc([C@@H](CC(=O)O)C4CC4)c3)c2CC(C)(C)Cn2cc(COCCOCCOCCOCCOCC(=O)NCC[N+](C)(CCC(=O)NCCN)CCC(=O)NCCN)nn2)c1]

MS (ESI) m/z 1097.3 [M]⁺. ¹H NMR (400 MHZ, DMSO-d₆): δ 8.36 (t, NH, 2H), 8.07 (t, NH, 1H), 7.93 (s, 1H), 7.87 (br, 6H), 7.46-7.41 (t, 1H), 7.26-7.17 (m, 3H), 6.93 (s, 1H), 6.91-6.87 (m, 2H), 5.18 (s, 2H), 4.49 (s, 2H), 4.18 (s, 2H), 3.92 (s, 2H), 3.78 (s, 3H), 3.68-3.50 (m, 16H), 3.62-3.58 (m, 4H), 3.52-3.48 (m, 2H), 3.38-3.29 (m, 6H), 3.04 (s, 3H), 2.90-2.86 (m, 4H), 2.69-2.59 (m, 7H), 2.52-2.49 (m, 1H), 2.33-2.24 (m, 1H), 1.02-0.97 (m, 1H), 0.61 (br, 6H), 0.52-0.47 (m, 1H), 0.33-0.24 (m, 2H), 0.15-0.10 (m, 1H).

### Example 183

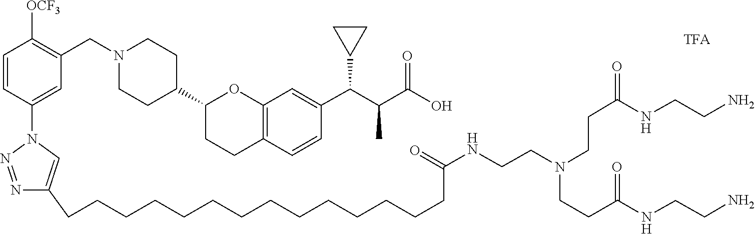 [SMILES: C[C@H](C(=O)O)[C@H](c1ccc2c(c1)O[C@@H](C1CCN(Cc3cc(-n4cc(CCCCCCCCCCCCCCC(=O)NCCN(CCC(=O)NCCN)CCC(=O)NCCN)nn4)ccc3OC(F)(F)F)CC1)CC2)C1CC1]

MS (ESI) m/z 1095.4 [M+H]⁺. ¹H NMR (400 MHZ, DMSO-d₆): δ 8.6 (s, 1H), 8.39 (brs, 2H), 8.13-8.11 (d, 1H), 8.09-8.08 (t, 1H), 7.87 (br, 6H), 7.74-7.72 (d, 1H), 6.97-6.95 (d, 1H), 6.66-6.64 (d, 1H), 6.51 (s, 1H), 4.47 (s, 2H), 3.82-3.80 (m, 1H), 3.50-3.30 (m, 12H), 3.23-3.13 (m, 4H), 2.90-2.87 (m, 4H), 2.75-2.63 (m, 9H), 2.10-2.06 (m, 2H), 2.00-1.78 (m, 6H), 1.70-1.65 (m, 4H), 1.49-1.46 (m, 2H), 1.37-1.26 (m, 20H), 1.09-1.04 (m, 1H), 0.82-0.80 (d, 3H), 0.52-0.49 (m, 1H), 0.28-0.19 (m, 2H), −0.05 ˜−0.10 (m, 1H).

### Example 184

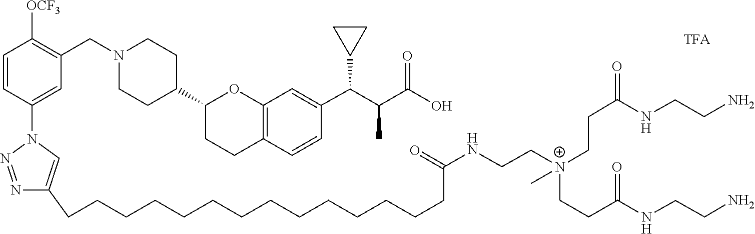 [SMILES: C[C@H](C(=O)O)[C@H](c1ccc2c(c1)O[C@@H](C1CCN(Cc3cc(-n4cc(CCCCCCCCCCCCCCC(=O)NCC[N+](C)(CCC(=O)NCCN)CCC(=O)NCCN)nn4)ccc3OC(F)(F)F)CC1)CC2)C1CC1]

MS (ESI) m/z 1034.4 [M]⁺. ¹H NMR (400 MHZ, DMSO-d₆): δ 8.59 (m, 1H), 8.42 (t, NH, 2H), 8.37 (s, 1H), 8.18 (t, NH, 1H), 8.10-8.08 (d, 1H), 7.85 (br, NH₃, 6H), 7.73-7.71 (d, 1H), 6.97-6.95 (d, 1H), 6.65-6.63 (d, 1H), 6.51 (s, 1H), 4.45 (s, 2H), 3.89 (br, 1H), 3.60-3.58 (m, 4H), 3.45-3.36 (m, 4H), 3.36-3.31 (m, 6H), 3.17-3.15 (br, 2H), 3.03 (s, 3H), 2.90-2.86 (m, 4H), 2.74-2.63 (m, 9H), 2.13-2.06 (m, 2H), 2.00-1.78 (m, 6H), 1.70-1.65 (m, 4H), 1.53-1.48 (m, 2H), 1.37-1.26 (m, 20H), 1.09-1.04 (m, 1H), 0.82-0.80 (d, 3H), 0.53-0.49 (m, 1H), 0.28-0.19 (m, 2H), −0.05 ˜−0.10 (m, 1H).

### Example 185

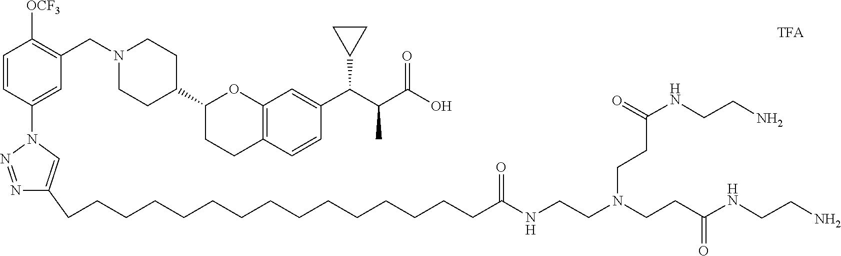 [SMILES: C[C@H](C(=O)O)[C@H](c1ccc2c(c1)O[C@@H](C1CCN(Cc3cc(-n4cc(CCCCCCCCCCCCCCCC(=O)NCCN(CCC(=O)NCCN)CCC(=O)NCCN)nn4)ccc3OC(F)(F)F)CC1)CC2)C1CC1]

MS (ESI) m/z 1111.4 [M]⁺. ¹H NMR (400 MHZ, DMSO-d₆): δ 8.59 (s, 1H), 8.44-8.39 (brs, 3H), 8.15 (t, NH, 1H), 8.12-8.09 (dd, 1H), 7.93 (br, 6H), 7.74-7.72 (dd, 1H), 6.97-6.95 (d, 1H), 6.66-6.64 (d, 1H), 6.51 (s, 1H), 4.47 (s, 2H), 3.82-3.79 (m, 1H), 3.43-3.36 (m, 6H), 3.34-3.29 (m, 6H), 3.20-3.15 (m, 4H), 2.90-2.87 (m, 4H), 2.75-2.63 (m, 9H), 2.10-2.06 (m, 2H), 1.95-1.78 (m, 6H), 1.70-1.65 (m, 4H), 1.49-1.46 (m, 2H), 1.37-1.26 (m, 22H), 1.09-1.04 (m, 1H), 0.82-0.80 (d, 3H), 0.52-0.49 (m, 1H), 0.28-0.19 (m, 2H), −0.05 ˜−0.10 (m, 1H).

### Example 186

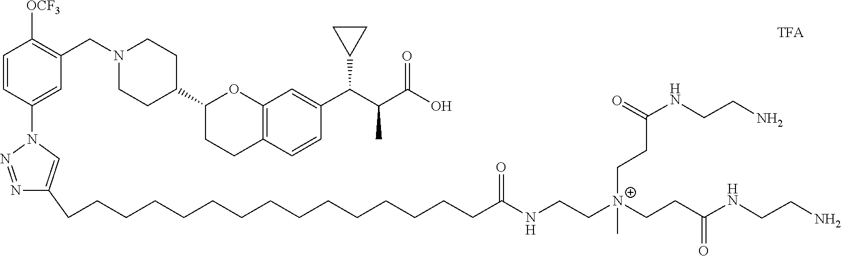 [SMILES: C[C@H](C(=O)O)[C@H](c1ccc2c(c1)O[C@@H](C1CCN(Cc3cc(-n4cc(CCCCCCCCCCCCCCCC(=O)NCC[N+](C)(CCC(=O)NCCN)CCC(=O)NCCN)nn4)ccc3OC(F)(F)F)CC1)CC2)C1CC1]

MS (ESI) m/z 1123.4 [M]⁺. ¹H NMR (400 MHz, DMSO-d₆): δ 8.61 (m, 2H), 8.40-8.37 (m, 3H), 8.16 (t, NH, 1H), 8.11-8.09 (d, 1H), 7.90 (br, NH₃, 6H), 7.74-7.72 (d, 1H), 6.97-6.95 (d, 1H), 6.65-6.63 (d, 1H), 6.51 (s, 1H), 4.46 (s, 2H), 3.81 (br, 1H), 3.60-3.56 (m, 4H), 3.45-3.36 (m, 4H), 3.35-3.31 (m, 6H), 3.17-3.15 (br, 2H), 3.03 (s, 3H), 2.90-2.86 (m, 4H), 2.74-2.63 (m, 9H), 2.10-2.06 (m, 2H), 2.00-1.78 (m, 6H), 1.70-1.65 (m, 4H), 1.50-1.47 (m, 2H), 1.37-1.26 (m, 22H), 1.09-1.04 (m, 1H), 0.82-0.80 (d, 3H), 0.53-0.49 (m, 1H), 0.28-0.19 (m, 2H), −0.05 ˜−0.10 (m, 1H).

### Example 187

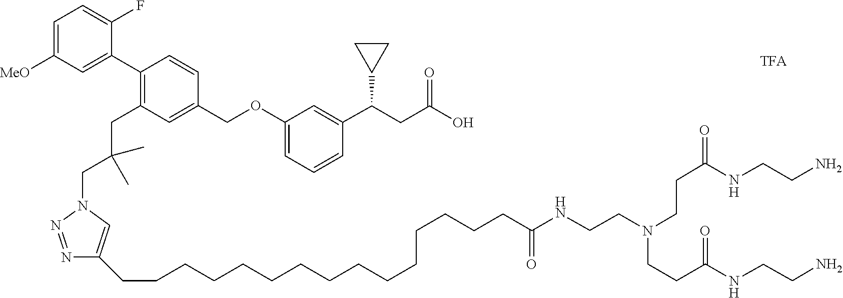 [SMILES: COc1ccc(F)c(-c2ccc(COc3cccc([C@@H](CC(=O)O)C4CC4)c3)cc2CC(C)(C)Cn2cc(CCCCCCCCCCCCCCCC(=O)NCCN(CCC(=O)NCCN)CCC(=O)NCCN)nn2)c1]

MS (ESI) m/z 1082.5 [M+H]⁺. ¹H NMR (400 MHZ, DMSO-d₆): δ 8.42 (t, NH, 2H), 8.15 (t, NH, 1H), 7.91 (br, 6H), 7.62 (s, 1H), 7.43 (s, 1H), 7.41-7.39 (d, 1H), 7.23-7.13 (m, 3H), 6.94-6.77 (m, 5H), 5.13 (s, 2H), 3.90 (s, 2H), 3.73 (s, 3H), 3.42-3.36 (m, 6H), 3.33-3.29 (m, 4H), 3.19-3.15 (m, 2H), 2.89-2.86 (m, 4H), 2.73-2.67 (m, 7H), 2.63-2.58 (m, 3H), 2.27-2.23 (m, 1H), 2.10-2.06 (t, 2H), 1.55-1.46 (m, 4H), 1.30-1.20 (m, 22H), 1.00-0.97 (m, 1H), 0.55-0.46 (m, 7H), 0.33-0.23 (m, 2H), 0.12-0.10 (m, 1H).

### Example 188

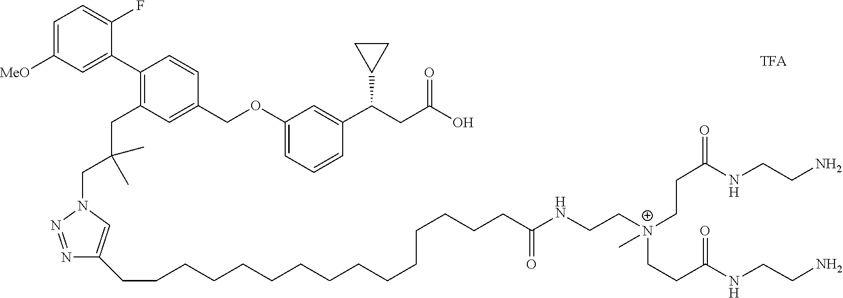 [SMILES: COc1ccc(F)c(-c2ccc(COc3cccc([C@@H](CC(=O)O)C4CC4)c3)cc2CC(C)(C)Cn2cc(CCCCCCCCCCCCCCCC(=O)NCC[N+](C)(CCC(=O)NCCN)CCC(=O)NCCN)nn2)c1]

MS (ESI) m/z 1096.5 [M]⁺. ¹H NMR (400 MHZ, DMSO-d₆): δ 8.40 (t, NH, 2H), 8.17 (t, NH, 1H), 7.94 (br, 6H), 7.62 (s, 1H), 7.43 (s, 1H), 7.41-7.39 (d, 1H), 7.23-7.11 (m, 3H), 6.94-6.91 (m, 2H), 6.86-6.77 (m, 3H), 5.13 (s, 2H), 4.14 (s, 2H), 3.73 (s, 3H), 3.60-3.56 (m, 4H), 3.46-3.43 (m, 2H), 3.34-3.30 (m, 6H), 3.03 (s, 3H), 2.90-2.86 (m, 4H), 2.73-2.59 (m, 7H), 2.56-2.50 (t, 3H), 2.26-2.23 (m, 1H), 2.10-2.06 (t, 2H), 1.55-1.47 (m, 4H), 1.28-1.23 (m, 22H), 1.00-0.97 (m, 1H), 0.55-0.46 (m, 7H), 0.33-0.23 (m, 2H), 0.12-0.10 (m, 1H).

### Example 189

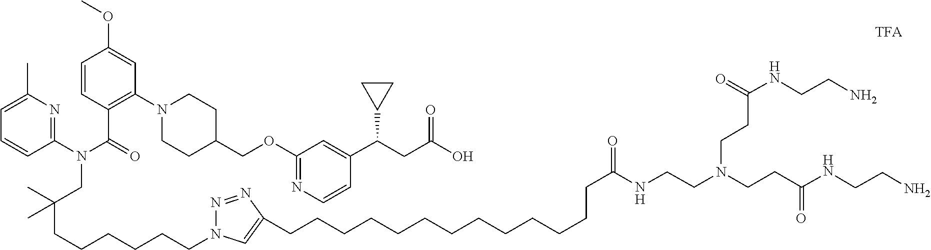 [SMILES: COc1ccc(C(=O)N(CC(C)(C)CCCCCCn2cc(CCCCCCCCCCCCCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

MS (ESI) m/z 1248.6 [M+H]⁺. ¹H NMR (400 MHZ, DMSO-d₆): δ 8.39 (t, NH, 2H), 8.13 (t, NH, 1H), 8.05-8.03 (d, 1H), 7.86 (br, NH₃, 6H), 7.79 (s, 1H), 7.28-7.25 (m, 1H), 7.11-7.09 (d, 1H), 6.93-6.91 (d, 1H), 6.85-6.83 (d, 1H), 6.71 (s, 1H), 6.53-6.50 (d, 1H), 6.42 (br, 1H), 6.22 (s, 1H), 4.24-4.21 (t, 2H), 4.13-4.11 (d, 2H), 4.08 (br, 2H), 3.68 (s, 3H), 3.42-3.36 (m, 6H), 3.33-3.28 (m, 4H), 3.18-3.15 (m, 2H), 2.90-2.85 (m, 4H), 2.69-2.64 (d, 2H), 2.64-2.55 (m, 6H), 2.33 (s, 3H), 2.25-2.22 (m, 1H), 2.10-2.06 (t, 2H), 1.75-1.66 (m, 5H), 1.57-1.55 (m, 2H), 1.49-1.46 (m, 2H), 1.39 (br, 2H), 1.25 (br, 18H), 1.20-0.90 (m, 9H), 0.73 (s, 6H), 0.54-0.48 (m, 1H), 0.32-0.25 (m, 2H), 0.20-0.15 (m, 1H).

### Example 190

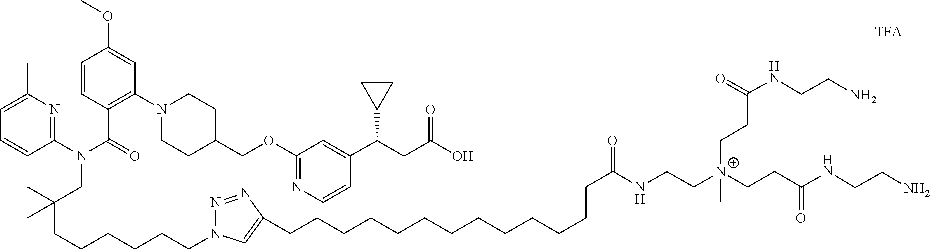 [SMILES: COc1ccc(C(=O)N(CC(C)(C)CCCCCCn2cc(CCCCCCCCCCCCCC(=O)NCC[N+](C)(CCC(=O)NCCN)CCC(=O)NCCN)nn2)c2cccc(C)n2)c(N2CCC(COc3cc([C@@H](CC(=O)O)C4CC4)ccn3)CC2)c1]

MS (ESI) m/z 1263.6 [M]⁺. ¹H NMR (400 MHZ, DMSO-d₆): δ 8.39 (t, NH, 2H), 8.16 (t, NH, 1H), 8.05-8.04 (d, 1H), 7.89 (br, NH₃, 6H), 7.80 (s, 1H), 7.28-7.25 (br, 1H), 7.11-7.09 (d, 1H), 6.93-6.92 (d, 1H), 6.85-6.83 (d, 1H), 6.72 (s, 1H), 6.53-6.51 (d, 1H), 6.41 (br, 1H), 6.22 (s, 1H), 4.24-4.21 (t, 2H), 4.16-4.12 (d, 2H), 4.09 (br, 2H), 3.69 (s, 3H), 3.59-3.56 (m, 4H), 3.46-3.44 (m, 2H), 3.34-3.29 (m, 4H), 3.03 (s, 3H), 2.90-2.85 (m, 4H), 2.69-2.65 (m, 6H), 2.59-2.55 (t, 2H), 2.33 (s, 3H), 2.27-2.21 (m, 1H), 2.10-2.07 (t, 2H), 1.75-1.66 (m, 5H), 1.57-1.55 (m, 2H), 1.49-1.46 (m, 2H), 1.39 (br, 2H), 1.25 (br, 18H), 1.11-0.94 (m, 9H), 0.75 (s, 6H), 0.53-0.46 (m, 1H), 0.32-0.25 (m, 2H), 0.18-0.14 (m, 1H).

### Example 191

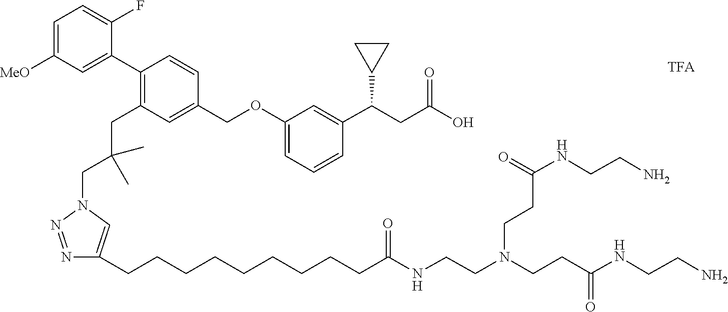 [SMILES: COc1ccc(F)c(-c2ccc(COc3cccc([C@@H](CC(=O)O)C4CC4)c3)cc2CC(C)(C)Cn2cc(CCCCCCCCCC(=O)NCCN(CCC(=O)NCCN)CCC(=O)NCCN)nn2)c1]

MS (ESI) m/z 998.4 [M+H]⁺. ¹H NMR (400 MHZ, DMSO-d₆): δ 8.42 (t, NH, 2H), 8.15 (t, NH, 1H), 7.92 (br, 6H), 7.62 (s, 1H), 7.44 (s, 1H), 7.41-7.39 (d, 1H), 7.23-7.12 (m, 3H), 6.95-6.91 (m, 2H), 6.86-6.2 (m, 2H), 6.79-6.77 (m, 1H), 5.14 (s, 2H), 3.98 (s, 2H), 3.73 (s, 3H), 3.42-3.37 (m, 6H), 3.34-3.29 (m, 4H), 3.19-3.15 (m, 2H), 2.91-2.86 (m, 4H), 2.73-2.61 (m, 7H), 2.60-2.53 (m, 3H), 2.27-2.23 (m, 1H), 2.10-2.07 (t, 2H), 1.56-1.46 (m, 4H), 1.30-1.20 (m, 10H), 1.01-0.97 (m, 1H), 0.55-0.48 (m, 7H), 0.33-0.23 (m, 2H), 0.12-0.10 (m, 1H).

### Example 192

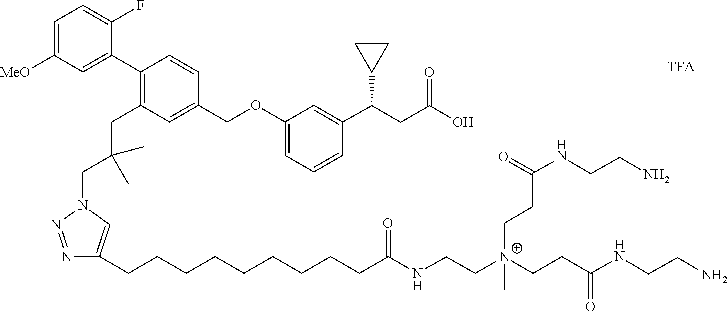 [SMILES: COc1ccc(F)c(-c2ccc(COc3cccc([C@@H](CC(=O)O)C4CC4)c3)cc2CC(C)(C)Cn2cc(CCCCCCCCCC(=O)NCC[N+](C)(CCC(=O)NCCN)CCC(=O)NCCN)nn2)c1]

MS (ESI) m/z 1012.5 [M]⁺. ¹H NMR (400 MHZ, DMSO-d₆): δ 8.40 (t, NH, 2H), 8.17 (t, NH, 1H), 7.93 (br, 6H), 7.62 (s, 1H), 7.44 (s, 1H), 7.41-7.39 (d, 1H), 7.23-7.12 (m, 3H), 6.94-6.91 (m, 2H), 6.86-6.77 (m, 3H), 5.14 (s, 2H), 3.98 (s, 2H), 3.73 (s, 3H), 3.60-3.56 (m, 4H), 3.46-3.43 (m, 2H), 3.34-3.30 (m, 6H), 3.04 (s, 3H), 2.90-2.86 (m, 4H), 2.70-2.61 (m, 7H), 2.57-2.53 (t, 3H), 2.28-2.22 (m, 1H), 2.11-2.07 (t, 2H), 1.55-1.47 (m, 4H), 1.28-1.23 (m, 22H), 1.00-0.97 (m, 1H), 0.55-0.46 (m, 7H), 0.33-0.23 (m, 2H), 0.12-0.10 (m, 1H).

### Example 193

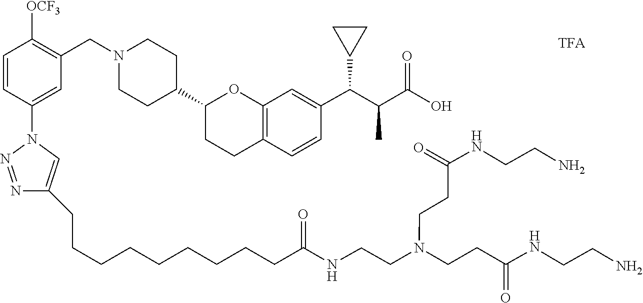 [SMILES: C[C@H](C(=O)O)[C@H](c1ccc2c(c1)O[C@@H](C1CCN(Cc3cc(-n4cc(CCCCCCCCCC(=O)NCCN(CCC(=O)NCCN)CCC(=O)NCCN)nn4)ccc3OC(F)(F)F)CC1)CC2)C1CC1]

MS (ESI) m/z 1025.4 [M+H]⁺. ¹H NMR (400 MHZ, DMSO-d₆): δ 8.59 (s, 1H), 8.43-8.39 (m, 2H), 8.15 (t, 1H), 8.12-8.09 (dd, 1H), 7.91 (br, 6H), 7.74-7.72 (dd, 1H), 6.97-6.95 (d, 1H), 6.66-6.64 (d, 1H), 6.51 (s, 1H), 4.47 (s, 2H), 3.82-3.81 (m, 1H), 3.45-3.35 (m, 7H), 3.34-3.29 (m, 4H), 3.18-3.15 (m, 4H), 2.90-2.87 (m, 4H), 2.73-2.69 (t, 2H), 2.64-2.61 (m, 7H), 2.11-2.07 (m, 2H), 1.95-1.82 (m, 6H), 1.68-1.63 (m, 4H), 1.52-1.47 (m, 2H), 1.35-1.22 (m, 10H), 1.09-1.04 (m, 1H), 0.82-0.80 (d, 3H), 0.52-0.49 (m, 1H), 0.25-0.17 (m, 2H), -0.08 ˜−0.12 (m, 1H).

### Example 194

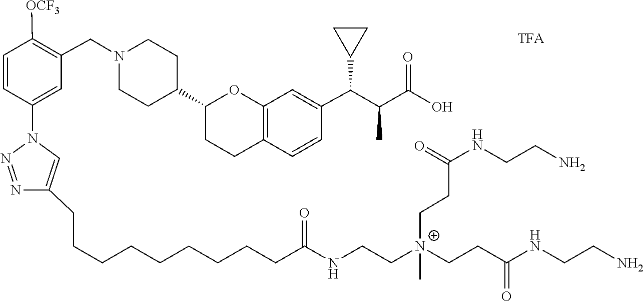 [SMILES: C[C@H](C(=O)O)[C@H](c1ccc2c(c1)O[C@@H](C1CCN(Cc3cc(-n4cc(CCCCCCCCCC(=O)NCC[N+](C)(CCC(=O)NCCN)CCC(=O)NCCN)nn4)ccc3OC(F)(F)F)CC1)CC2)C1CC1]

MS (ESI) m/z 1039.4 [M]⁺. ¹H NMR (400 MHZ, DMSO-d₆): δ 8.59 (m, 1H), 8.40-8.37 (m, 2H), 8.17 (t, NH, 1H), 8.12-8.09 (dd, 1H), 7.90 (br, NH₃, 6H), 7.75-7.72 (dd, 1H), 6.97-6.95 (d, 1H), 6.66-6.64 (d, 1H), 6.51 (s, 1H), 4.47 (s, 2H), 3.82-3.80 (m, 1H), 3.60-3.40 (m, 4H), 3.34-3.29 (m, 6H), 3.18-3.14 (br, 2H), 3.03 (s, 3H), 2.90-2.86 (m, 4H), 2.71-2.62 (m, 9H), 2.11-2.07 (m, 2H), 1.89-1.82 (m, 6H), 1.68-1.63 (m, 4H), 1.50-1.47 (m, 2H), 1.35-1.22 (m, 10H), 1.09-1.04 (m, 1H), 0.82-0.80 (d, 3H), 0.52-0.49 (m, 1H), 0.25-0.17 (m, 2H), −0.08 ˜−0.12 (m, 1H).

### Example 195

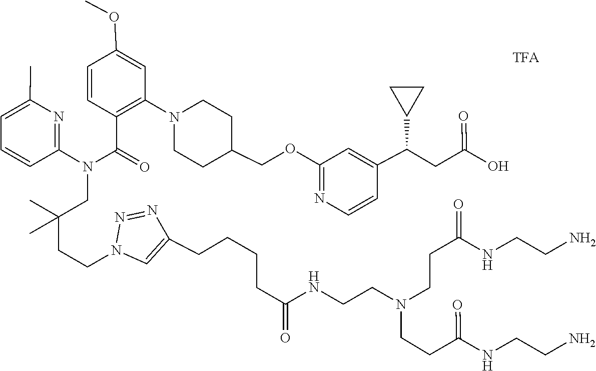 [SMILES: COc1ccc(C(=O)N(CC(C)(C)CCn2cc(CCCCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

MS (ESI) m/z 1066.4 [M+H]⁺. ¹H NMR (400 MHZ, DMSO-d₆): δ 8.41 (t, NH, 2H), 8.19 (t, NH, 1H), 8.06-8.05 (d, 1H), 7.89 (br, NH₃, 6H), 7.71 (s, 1H), 7.30-7.27 (t, 1H), 7.18-7.16 (d, 1H), 6.95-6.93 (dd, 1H), 6.87-6.86 (d, 1H), 6.74 (s, 1H), 6.56-6.53 (dd, 1H), 6.47 (br, 1H), 6.22 (s, 1H), 4.37-4.32 (t, 2H), 4.20 (br, 2H), 4.14-4.12 (d, 2H), 3.69 (s, 3H), 3.43-3.37 (m, 6H), 3.34-3.30 (m, 4H), 3.20-3.18 (br, 2H), 2.91-2.86 (m, 4H), 2.70-2.69 (d, 2H), 2.65-2.57 (m, 6H), 2.37 (s, 3H), 2.27-2.21 (m, 1H), 2.16-2.13 (t, 2H), 1.77-1.66 (m, 5H), 1.58-1.47 (m, 4H), 1.35 (br, 2H), 1.03-0.96 (m, 1H), 0.77 (s, 6H), 0.54-0.50 (m, 1H), 0.32-0.25 (m, 2H), 0.23-0.18 (m, 1H).

### Example 196

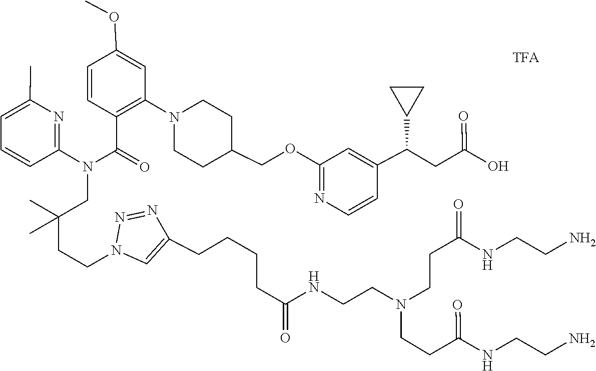 [SMILES: COc1ccc(C(=O)N(CC(C)(C)CCn2cc(CCCCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

MS (ESI) m/z 1094.4 [M+H]⁺. ¹H NMR (400 MHZ, DMSO-d₆): δ 8.41 (t, NH, 2H), 8.16 (t, NH, 1H), 8.06-8.05 (d, 1H), 7.88 (br, NH₃, 6H), 7.71 (s, 1H), 7.31-7.27 (t, 1H), 7.18-7.16 (d, 1H), 6.94-6.93 (dd, 1H), 6.87-6.85 (d, 1H), 6.74 (s, 1H), 6.55-6.52 (dd, 1H), 6.47 (br, 1H), 6.22 (s, 1H), 4.37-4.32 (t, 2H), 4.20 (br, 2H), 4.14-4.12 (d, 2H), 3.70 (s, 3H), 3.43-3.37 (m, 6H), 3.34-3.30 (m, 4H), 3.20-3.16 (br, 2H), 2.91-2.86 (m, 4H), 2.70-2.69 (d, 2H), 2.65-2.62 (t, 4H), 2.59-2.56 (t, 2H), 2.37 (s, 3H), 2.27-2.21 (m, 1H), 2.12-2.08 (t, 2H), 1.76-1.66 (m, 5H), 1.58-1.48 (m, 4H), 1.35 (br, 2H), 1.29-1.25 (m, 4H), 1.03-0.96 (m, 1H), 0.77 (s, 6H), 0.54-0.50 (m, 1H), 0.32-0.25 (m, 2H), 0.23-0.18 (m, 1H).

### Example 197

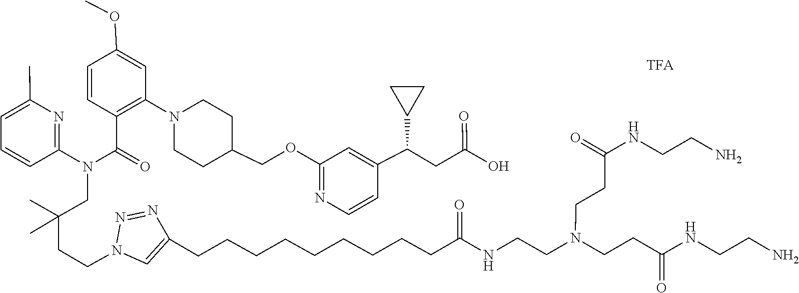 [SMILES: COc1ccc(C(=O)N(CC(C)(C)CCn2cc(CCCCCCCCCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

MS (ESI) m/z 1037.4 [M+H]⁺. ¹H NMR (400 MHZ, DMSO-d₆): δ 8.40 (t, NH, 2H), 8.15 (t, NH, 1H), 8.05-8.04 (d, 1H), 7.86 (br, NH₃, 6H), 7.70 (s, 1H), 7.30-7.26 (t, 1H), 7.17-7.15 (d, 1H), 6.93-6.92 (dd, 1H), 6.86-6.85 (d, 1H), 6.72 (s, 1H), 6.54-6.52 (dd, 1H), 6.47 (br, 1H), 6.22 (s, 1H), 4.39-4.34 (t, 2H), 4.19 (br, 2H), 4.13-4.12 (d, 2H), 3.70 (s, 3H), 3.42-3.36 (m, 6H), 3.33-3.29 (m, 4H), 3.19-3.15 (br, 2H), 2.90-2.86 (m, 4H), 2.70-2.68 (d, 2H), 2.64-2.61 (t, 4H), 2.59-2.55 (t, 2H), 2.36 (s, 3H), 2.27-2.21 (m, 1H), 2.11-2.07 (t, 2H), 1.76-1.66 (m, 5H), 1.57-1.46 (m, 4H), 1.35 (br, 2H), 1.27-1.24 (m, 10H), 1.01-0.97 (m, 1H), 0.77 (s, 6H), 0.54-0.50 (m, 1H), 0.32-0.25 (m, 2H), 0.23-0.18 (m, 1H).

### Example 198

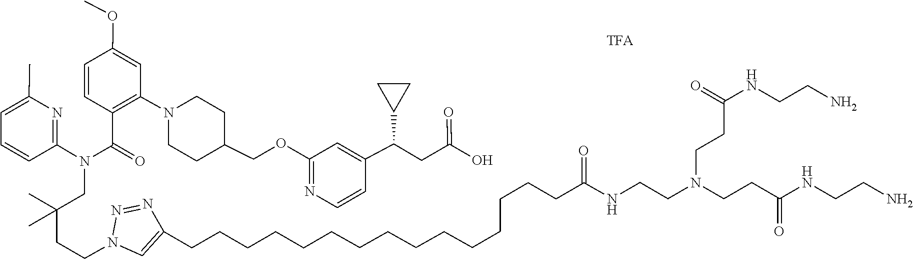 [SMILES: COc1ccc(C(=O)N(CC(C)(C)CCn2cc(CCCCCCCCCCCCCCCC(=O)NCCN(CCC(=O)NCCN)CCC(=O)NCCN)nn2)c2cccc(C)n2)c(N2CCC(COc3cc([C@@H](CC(=O)O)C4CC4)ccn3)CC2)c1]

MS (ESI) m/z 1220.5 [M+H]⁺. ¹H NMR (400 MHZ, DMSO-d₆): δ 8.37 (t, NH, 2H), 8.13 (t, NH, 1H), 8.05-8.03 (d, 1H), 7.82 (br, NH₃, 6H), 7.68 (s, 1H), 7.30-7.26 (t, 1H), 7.16-7.14 (d, 1H), 6.93-6.91 (dd, 1H), 6.86-6.84 (d, 1H), 6.71 (s, 1H), 6.54-6.51 (dd, 1H), 6.47 (br, 1H), 6.21 (s, 1H), 4.33 (br, 2H), 4.19 (br, 2H), 4.13-4.12 (d, 2H), 3.70 (s, 3H), 3.42-3.36 (m, 6H), 3.33-3.28 (m, 4H), 3.19-3.15 (br, 2H), 2.90-2.85 (m, 4H), 2.69-2.67 (d, 2H), 2.64-2.60 (t, 4H), 2.58-2.55 (t, 2H), 2.36 (s, 3H), 2.27-2.21 (m, 1H), 2.10-2.07 (t, 2H), 1.76-1.66 (m, 5H), 1.57-1.46 (m, 4H), 1.35 (br, 2H), 1.30-1.18 (m, 22H), 1.02-0.97 (m, 1H), 0.77 (s, 6H), 0.54-0.50 (m, 1H), 0.32-0.25 (m, 2H), 0.23-0.18 (m, 1H).

### Example 199

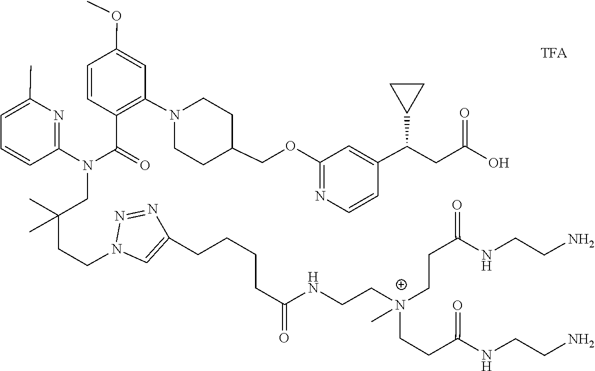 [SMILES: COc1ccc(C(=O)N(CC(C)(C)CCn2cc(CCCCC(=O)NCC[N+](C)(CCC(=O)NCCN)CCC(=O)NCCN)nn2)c2cccc(C)n2)c(N2CCC(COc3cc([C@@H](CC(=O)O)C4CC4)ccn3)CC2)c1]

MS (ESI) m/z 1080.4 [M]⁺. ¹H NMR (400 MHZ, DMSO-d₆): δ 8.38 (t, NH, 2H), 8.20 (t, NH, 1H), 8.06-8.05 (d, 1H), 7.89 (br, NH₃, 6H), 7.71 (s, 1H), 7.31-7.27 (br, 1H), 7.18-7.16 (d, 1H), 6.94-6.93 (dd, 1H), 6.87-6.85 (d, 1H), 6.73 (s, 1H), 6.55-6.53 (dd, 1H), 6.46-6.45 (br, 1H), 6.22 (s, 1H), 4.32-4.30 (t, 2H), 4.13-4.11 (d, 2H), 4.12 (br, 2H), 3.69 (s, 3H), 3.60-3.56 (m, 4H), 3.47-3.45 (m, 2H), 3.34-3.29 (m, 6H), 3.04 (s, 3H), 2.92-2.86 (m, 4H), 2.71−−2.67 (m, 6H), 2.60-2.57 (t, 2H), 2.36 (s, 3H), 2.27-2.21 (m, 1H), 2.16-2.13 (t, 2H), 1.76-1.66 (m, 5H), 1.56-1.53 (m, 4H), 1.35 (br, 2H), 1.04-0.94 (m, 1H), 0.77 (s, 6H), 0.53-0.48 (m, 1H), 0.32-0.25 (m, 2H), 0.23-0.18 (m, 1H).

### Example 200

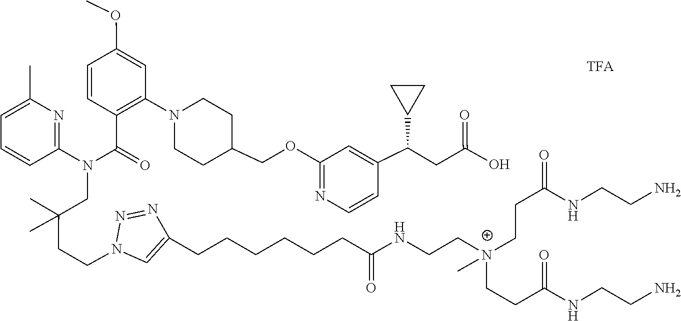 [SMILES: COc1ccc(C(=O)N(CC(C)(C)CCn2cc(CCCCCCC(=O)NCC[N+](C)(CCC(=O)NCCN)CCC(=O)NCCN)nn2)c2cccc(C)n2)c(N2CCC(COc3cc([C@@H](CC(=O)O)C4CC4)ccn3)CC2)c1]

MS (ESI) m/z 1109.4 [M]⁺. ¹H NMR (400 MHZ, DMSO-d₆): δ 8.39 (t, NH, 2H), 8.18 (t, NH, 1H), 8.06-8.04 (d, 1H), 7.90 (br, NH₃, 6H), 7.71 (s, 1H), 7.31-7.27 (br, 1H), 7.17-7.15 (d, 1H), 6.94-6.93 (dd, 1H), 6.87-6.85 (d, 1H), 6.73 (s, 1H), 6.55-6.52 (dd, 1H), 6.46-6.45 (br, 1H), 6.22 (s, 1H), 4.32-4.30 (t, 2H), 4.12 (br, 2H), 4.14-4.12 (d, 2H), 3.69 (s, 3H), 3.60-3.56 (m, 4H), 3.47-3.45 (m, 2H), 3.34-3.29 (m, 6H), 3.04 (s, 3H), 2.90-2.86 (m, 4H), 2.70−−2.66 (m, 6H), 2.59-2.55 (t, 2H), 2.36 (s, 3H), 2.27-2.21 (m, 1H), 2.12-2.08 (t, 2H), 1.76-1.66 (m, 5H), 1.57-1.48 (m, 4H), 1.32-1.22 (m, 6H), 1.04-0.94 (m, 1H), 0.76 (s, 6H), 0.53-0.48 (m, 1H), 0.32-0.25 (m, 2H), 0.23-0.18 (m, 1H).

### Example 201

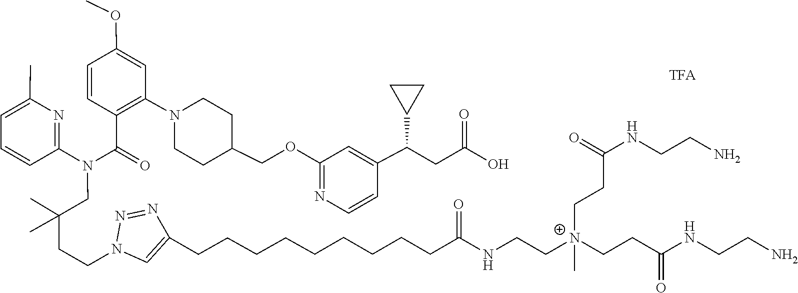 [SMILES: COc1ccc(C(=O)N(CC(C)(C)CCn2cc(CCCCCCCCCC(=O)NCC[N+](C)(CCC(=O)NCCN)CCC(=O)NCCN)nn2)c2cccc(C)n2)c(N2CCC(COc3cc([C@@H](CC(=O)O)C4CC4)ccn3)CC2)c1]

MS (ESI) m/z 1151.5 [M]⁺. ¹H NMR (400 MHZ, DMSO-d₆): δ 8.38 (t, NH, 2H), 8.17 (t, NH, 1H), 8.05-8.04 (d, 1H), 7.89 (br, NH₃, 6H), 7.70 (s, 1H), 7.30-7.26 (br, 1H), 7.17-7.15 (d, 1H), 6.93-6.92 (d, 1H), 6.87-6.85 (d, 1H), 6.72 (s, 1H), 6.54-6.51 (dd, 1H), 6.45-6.44 (br, 1H), 6.21 (s, 1H), 4.33 (br, 2H), 4.19 (br, 2H), 4.14-4.12 (d, 2H), 3.69 (s, 3H), 3.60-3.56 (m, 4H), 3.47-3.45 (m, 2H), 3.34-3.29 (m, 6H), 3.03 (s, 3H), 2.90-2.86 (m, 4H), 2.70--2.66 (m, 6H), 2.59-2.55 (t, 2H), 2.36 (s, 3H), 2.27-2.21 (m, 1H), 2.11-2.07 (t, 2H), 1.76-1.66 (m, 5H), 1.57-1.46 (m, 4H), 1.30-1.20 (m, 12H), 1.04-0.94 (m, 1H), 0.76 (s, 6H), 0.53-0.48 (m, 1H), 0.32-0.25 (m, 2H), 0.23-0.18 (m, 1H).

### Example 202

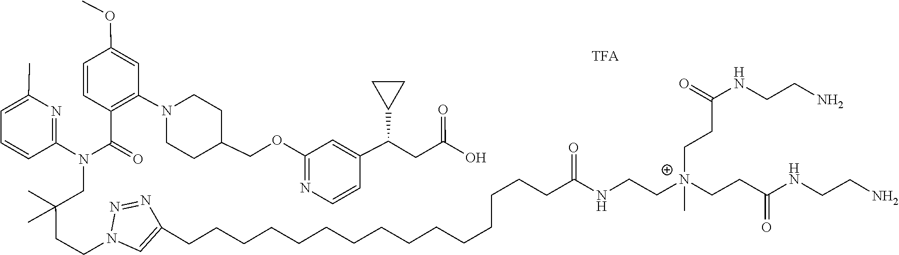 [SMILES: COc1ccc(C(=O)N(CC(C)(C)CCn2cc(CCCCCCCCCCCCCCCC(=O)NCC[N+](C)(CCC(=O)NCCN)CCC(=O)NCCN)nn2)c2cccc(C)n2)c(N2CCC(COc3cc([C@@H](CC(=O)O)C4CC4)ccn3)CC2)c1]

MS (ESI) m/z 1234.5 [M]⁺. ¹H NMR (400 MHZ, DMSO-d₆): δ 8.40 (t, NH, 2H), 8.17 (t, NH, 1H), 8.05-8.04 (d, 1H), 7.91 (br, NH₃, 6H), 7.69 (s, 1H), 7.30-7.26 (br, 1H), 7.17-7.14 (d, 1H), 6.93-6.92 (d, 1H), 6.86-6.84 (d, 1H), 6.72 (s, 1H), 6.54-6.51 (dd, 1H), 6.46 (br, 1H), 6.22 (s, 1H), 4.34 (br, 2H), 4.19 (br, 2H), 4.14-4.12 (d, 2H), 3.69 (s, 3H), 3.60-3.56 (m, 4H), 3.47-3.45 (m, 2H), 3.34-3.29 (m, 6H), 3.04 (s, 3H), 2.90-2.86 (m, 4H), 2.72−−2.66 (m, 6H), 2.58-2.55 (t, 2H), 2.36 (s, 3H), 2.27-2.21 (m, 1H), 2.11-2.07 (t, 2H), 1.76-1.65 (m, 5H), 1.57-1.46 (m, 4H), 1.30-1.20 (m, 24H), 1.04-0.94 (m, 1H), 0.76 (s, 6H), 0.53-0.48 (m, 1H), 0.32-0.25 (m, 2H), 0.23-0.18 (m, 1H).

### Example 203

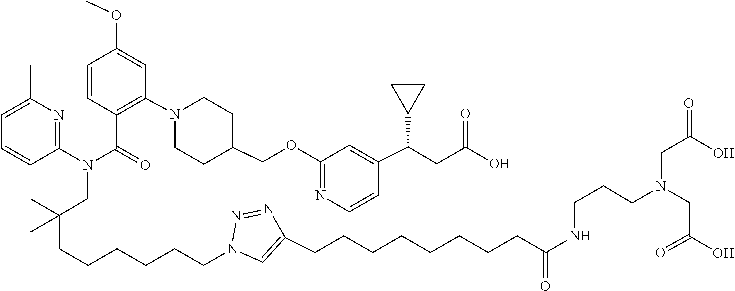 [SMILES: COc1ccc(C(=O)N(CC(C)(C)CCCCCCn2cc(CCCCCCCCC(=O)NCCCN(CC(=O)O)CC(=O)O)nn2)c2cccc(C)n2)c(N2CCC(COc3cc([C@@H](CC(=O)O)C4CC4)ccn3)CC2)c1]

MS (ESI) m/z 1080.4 [M+H]⁺. ¹H NMR (400 MHZ, DMSO-d₆): δ 8.05-8.04 (d, 1H), 7.84 (t, NH, 1H), 7.80 (s, 1H), 7.27-7.25 (m, 1H), 7.11-7.09 (d, 1H), 6.93-6.91 (dd, 1H), 6.85-6.83 (d, 1H), 6.71 (s, 1H), 6.53-6.50 (dd, 1H), 6.42 (br, 1H), 6.22 (br, 1H), 4.24-4.21 (t, 2H), 4.13-4.12 (d, 2H), 4.08 (br, 2H), 4.03 (s, 4H), 3.69 (s, 3H), 3.15-3.11 (m, 2H), 3.09-3.04 (m, 2H), 2.69-2.68 (d, 2H), 2.59-2.56 (t, 2H), 2.34 (s, 3H), 2.27-2.21 (m, 1H), 2.05-2.02 (t, 2H), 1.76-1.66 (m, 7H), 1.58-1.54 (t, 2H), 1.48-1.45 (t, 2H), 1.38-1.20 (m, 10H), 1.11-1.05 (m, 4H), 1.03-0.93 (m, 5H), 0.75 (s, 6H), 0.52-0.47 (m, 1H), 0.33-0.26 (m, 2H), 0.18-0.14 (m, 1H).

### Example 204

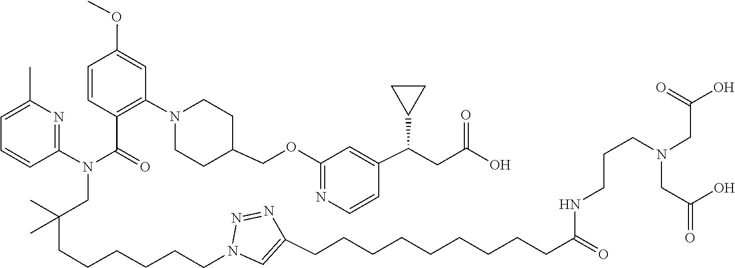 [SMILES: COc1ccc(C(=O)N(CC(C)(C)CCCCCCn2cc(CCCCCCCCCC(=O)NCCCN(CC(=O)O)CC(=O)O)nn2)c2cccc(C)n2)c(N2CCC(COc3cc([C@@H](CC(=O)O)C4CC4)ccn3)CC2)c1]

MS (ESI) m/z 1094.4 [M+H]⁺. ¹H NMR (400 MHZ, DMSO-d₆): δ 8.05-8.03 (d, 1H), 7.80 (s, 1H), 7.79-7.77 (t, NH, 1H), 7.26-7.24 (m, 1H), 7.11-7.09 (d, 1H), 6.92-6.91 (dd, 1H), 6.85-6.83 (d, 1H), 6.70 (s, 1H), 6.53-6.50 (dd, 1H), 6.42 (br, 1H), 6.22 (br, 1H), 4.24-4.21 (t, 2H), 4.13-4.11 (d, 2H), 4.01 (br, 2H), 3.69 (s, 3H), 3.64 (s, 4H), 3.08-3.03 (m, 2H), 2.82 (br, 2H), 2.69-2.67 (d, 2H), 2.59-2.55 (t, 2H), 2.33 (s, 3H), 2.26-2.21 (m, 1H), 2.04-2.01 (t, 2H), 1.76-1.65 (m, 7H), 1.57-1.54 (t, 2H), 1.49-1.44 (t, 2H), 1.36-1.20 (m, 12H), 1.11-1.05 (m, 4H), 1.03-0.95 (m, 5H), 0.75 (s, 6H), 0.53-0.48 (m, 1H), 0.34-0.26 (m, 2H), 0.18-0.14 (m, 1H).

### Example 205

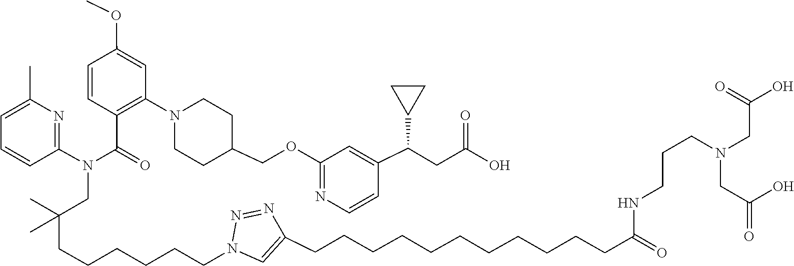 [SMILES: COc1ccc(C(=O)N(CC(C)(C)CCCCCCn2cc(CCCCCCCCCCCC(=O)NCCCN(CC(=O)O)CC(=O)O)nn2)c2cccc(C)n2)c(N2CCC(COc3cc([C@@H](CC(=O)O)C4CC4)ccn3)CC2)c1]

MS (ESI) m/z 1122.4 [M+H]⁺. ¹H NMR (400 MHZ, DMSO-d₆): δ 8.04-8.03 (d, 1H), 7.80 (br, 2H), 7.26-7.25 (m, 1H), 7.11-7.09 (d, 1H), 6.92-6.91 (dd, 1H), 6.85-6.83 (d, 1H), 6.70 (s, 1H), 6.53-6.50 (dd, 1H), 6.41 (br, 1H), 6.22 (br, 1H), 4.24-4.21 (t, 2H), 4.13-4.11 (d, 2H), 4.04 (br, 2H), 3.76 (br, 4H), 3.69 (s, 3H), 3.08-3.03 (m, 2H), 2.91 (br, 2H), 2.69-2.67 (d, 2H), 2.59-2.55 (t, 2H), 2.33 (s, 3H), 2.26-2.20 (m, 1H), 2.04-2.01 (t, 2H), 1.76-1.61 (m, 7H), 1.57-1.54 (t, 2H), 1.48-1.44 (t, 2H), 1.36-1.20 (m, 16H), 1.11-1.05 (m, 4H), 1.03-0.95 (m, 5H), 0.75 (s, 6H), 0.53-0.48 (m, 1H), 0.34-0.26 (m, 2H), 0.18-0.14 (m, 1H).

### Example 206

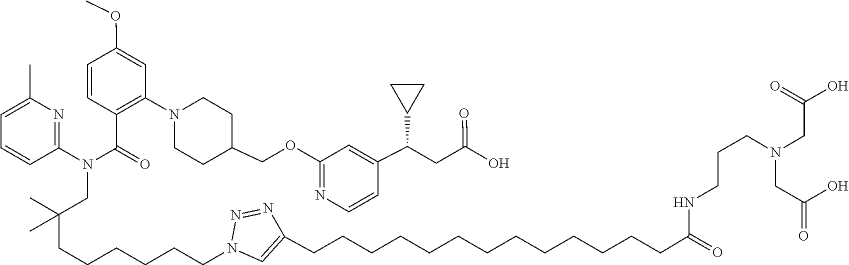 [SMILES: COc1ccc(C(=O)N(CC(C)(C)CCCCCCn2cc(CCCCCCCCCCCCCC(=O)NCCCN(CC(=O)O)CC(=O)O)nn2)c2cccc(C)n2)c(N2CCC(COc3cc([C@@H](CC(=O)O)C4CC4)ccn3)CC2)c1]

MS (ESI) m/z 1150.4 [M+H]⁺. ¹H NMR (400 MHZ, DMSO-d₆): δ 8.04-8.04 (d, 1H), 7.85 (br, 1H), 7.80 (s, 1H), 7.25 (br, 1H), 7.11-7.09 (d, 1H), 6.93-6.92 (dd, 1H), 6.85-6.83 (d, 1H), 6.72 (s, 1H), 6.53-6.50 (dd, 1H), 6.43 (br, 1H), 6.22 (br, 1H), 4.22-4.21 (t, 2H), 4.13-4.11 (d, 2H), 4.04 (br, 2H), 4.03 (br, 4H), 3.68 (s, 3H), 3.16 (br, 2H), 3.07-3.05 (m, 2H), 2.69-2.67 (d, 2H), 2.59-2.55 (t, 2H), 2.33 (s, 3H), 2.27-2.21 (m, 1H), 2.05-2.01 (t, 2H), 1.76-1.61 (m, 7H), 1.57-1.54 (t, 2H), 1.48-1.44 (t, 2H), 1.36-1.20 (m, 20H), 1.11-1.05 (m, 4H), 1.03-0.95 (m, 5H), 0.75 (s, 6H), 0.53-0.48 (m, 1H), 0.34-0.26 (m, 2H), 0.18-0.14 (m, 1H).

### Example 207

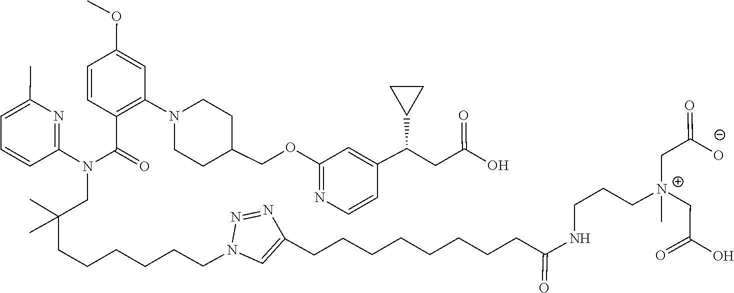 [SMILES: COc1ccc(C(=O)N(CC(C)(C)CCCCCCn2cc(CCCCCCCCC(=O)NCCC[N+](C)(CC(=O)[O-])CC(=O)O)nn2)c2cccc(C)n2)c(N2CCC(COc3cc([C@@H](CC(=O)O)C4CC4)ccn3)CC2)c1]

MS (ESI) m/z 1095.4 [M+H]⁺. ¹H NMR (400 MHZ, DMSO-d₆): δ 8.05-8.04 (d, 1H), 7.89 (t, 1H), 7.80 (s, 1H), 7.27-7.26 (m, 1H), 7.11-7.09 (d, 1H), 6.93-6.92 (dd, 1H), 6.85-6.84 (d, 1H), 6.72 (s, 1H), 6.53-6.50 (dd, 1H), 6.43 (br, 1H), 6.22 (br, 1H), 4.51 (s, 4H), 4.24-4.21 (t, 2H), 4.13-4.12 (d, 2H), 4.09 (br, 2H), 3.69 (s, 3H), 3.67-3.63 (m, 2H), 3.31 (s, 3H), 3.11-3.06 (m, 2H), 2.70-2.68 (d, 2H), 2.59-2.56 (t, 2H), 2.34 (s, 3H), 2.27-2.21 (m, 1H), 2.06-2.02 (t, 2H), 1.85-1.80 (m, 2H), 1.76-1.66 (m, 5H), 1.58-1.54 (t, 2H), 1.49-1.44 (t, 2H), 1.36-1.20 (m, 10H), 1.11-1.05 (m, 4H), 1.03-0.95 (m, 5H), 0.75 (s, 6H), 0.53-0.48 (m, 1H), 0.34-0.26 (m, 2H), 0.18-0.14 (m, 1H).

### Example 208

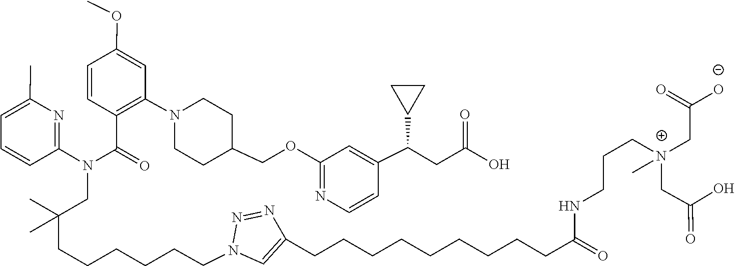 [SMILES: COc1ccc(C(=O)N(CC(C)(C)CCCCCCn2cc(CCCCCCCCCC(=O)NCCC[N+](C)(CC(=O)[O-])CC(=O)O)nn2)c2cccc(C)n2)c(N2CCC(COc3cc([C@@H](CC(=O)O)C4CC4)ccn3)CC2)c1]

MS (ESI) m/z 1109.4 [M+H]⁺. ¹H NMR (400 MHZ, DMSO-d₆): δ 8.05-8.04 (d, 1H), 7.88 (t, 1H), 7.80 (s, 1H), 7.27-7.25 (m, 1H), 7.11-7.09 (d, 1H), 6.94-6.92 (dd, 1H), 6.85-6.84 (d, 1H), 6.72 (s, 1H), 6.53-6.50 (dd, 1H), 6.43 (br, 1H), 6.23 (br, 1H), 4.50 (s, 4H), 4.24-4.21 (t, 2H), 4.14-4.12 (d, 2H), 4.08 (br, 2H), 3.69 (s, 3H), 3.67-3.63 (m, 2H), 3.31 (s, 3H), 3.11-3.06 (m, 2H), 2.70-2.68 (d, 2H), 2.59-2.56 (t, 2H), 2.34 (s, 3H), 2.27-2.21 (m, 1H), 2.05-2.02 (t, 2H), 1.85-1.80 (m, 2H), 1.76-1.66 (m, 5H), 1.58-1.54 (t, 2H), 1.49-1.44 (t, 2H), 1.36-1.20 (m, 12H), 1.11-1.05 (m, 4H), 1.03-0.95 (m, 5H), 0.75 (s, 6H), 0.53-0.48 (m, 1H), 0.34-0.26 (m, 2H), 0.18-0.14 (m, 1H).

### Example 209

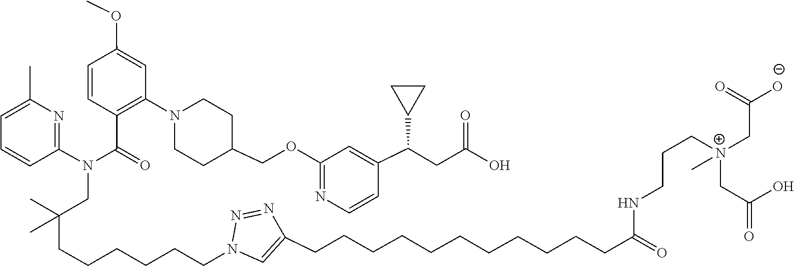 [SMILES: COc1ccc(C(=O)N(CC(C)(C)CCCCCCn2cc(CCCCCCCCCCCC(=O)NCCC[N+](C)(CC(=O)[O-])CC(=O)O)nn2)c2cccc(C)n2)c(N2CCC(COc3cc([C@@H](CC(=O)O)C4CC4)ccn3)CC2)c1]

MS (ESI) m/z 1136.4 [M+H]⁺. ¹H NMR (400 MHZ, DMSO-d₆): δ 8.05-8.04 (d, 1H), 7.88 (t, 1H), 7.80 (s, 1H), 7.28-7.25 (m, 1H), 7.11-7.09 (d, 1H), 6.94-6.92 (dd, 1H), 6.85-6.83 (d, 1H), 6.72 (s, 1H), 6.53-6.50 (dd, 1H), 6.43 (br, 1H), 6.22 (br, 1H), 4.46 (s, 4H), 4.24-4.21 (t, 2H), 4.14-4.12 (d, 2H), 4.09 (br, 2H), 3.69 (s, 3H), 3.67-3.63 (m, 2H), 3.31 (s, 3H), 3.11-3.06 (m, 2H), 2.69-2.67 (d, 2H), 2.59-2.55 (t, 2H), 2.34 (s, 3H), 2.27-2.21 (m, 1H), 2.05-2.02 (t, 2H), 1.85-1.80 (m, 2H), 1.76-1.66 (m, 5H), 1.58-1.54 (t, 2H), 1.49-1.44 (t, 2H), 1.36-1.20 (m, 16H), 1.11-1.05 (m, 4H), 1.03-0.95 (m, 5H), 0.75 (s, 6H), 0.53-0.48 (m, 1H), 0.34-0.26 (m, 2H), 0.18-0.14 (m, 1H).

### Example 210

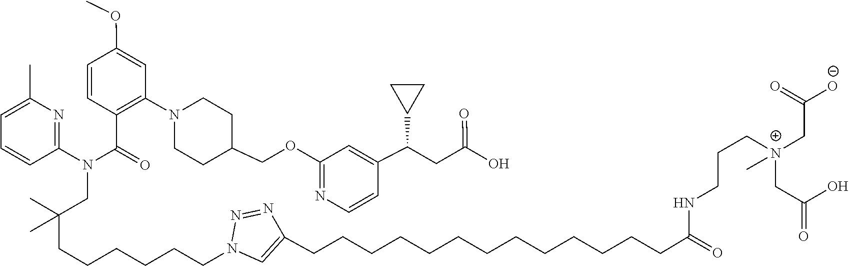 [SMILES: COc1ccc(C(=O)N(CC(C)(C)CCCCCCn2cc(CCCCCCCCCCCCCC(=O)NCCC[N+](C)(CC(=O)[O-])CC(=O)O)nn2)c2cccc(C)n2)c(N2CCC(COc3cc([C@@H](CC(=O)O)C4CC4)ccn3)CC2)c1]

MS (ESI) m/z 1165.4 [M+H]⁺. ¹H NMR (400 MHZ, DMSO-d₆): δ 8.05-8.04 (d, 1H), 7.89 (t, 1H), 7.80 (s, 1H), 7.26 (br, 1H), 7.11-7.09 (d, 1H), 6.93-6.92 (dd, 1H), 6.85-6.83 (d, 1H), 6.72 (s, 1H), 6.53-6.50 (dd, 1H), 6.42 (br, 1H), 6.23 (br, 1H), 4.47 (s, 4H), 4.24-4.21 (t, 2H), 4.14-4.12 (d, 2H), 4.09 (br, 2H), 3.69 (s, 3H), 3.67-3.63 (m, 2H), 3.31 (s, 3H), 3.11-3.06 (m, 2H), 2.70-2.68 (d, 2H), 2.59-2.56 (t, 2H), 2.34 (s, 3H), 2.27-2.21 (m, 1H), 2.06-2.02 (t, 2H), 1.86-1.80 (m, 2H), 1.73-1.66 (m, 5H), 1.58-1.54 (t, 2H), 1.49-1.44 (t, 2H), 1.36-1.20 (m, 20H), 1.11-1.05 (m, 4H), 1.03-0.95 (m, 5H), 0.75 (s, 6H), 0.53-0.48 (m, 1H), 0.34-0.26 (m, 2H), 0.18-0.14 (m, 1H).

### Example 211

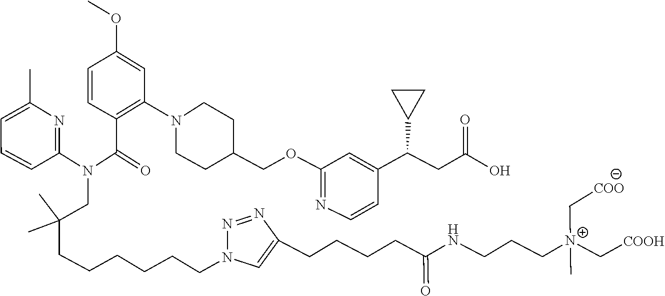

MS (ESI) m/z 1038.4 [M+H]⁺. ¹H NMR (400 MHZ, DMSO-d₆): δ 8.05-8.04 (d, 1H), 7.92 (t, 1H), 7.81 (s, 1H), 7.29-7.25 (m, 1H), 7.11-7.09 (d, 1H), 6.93-6.92 (dd, 1H), 6.85-6.84 (d, 1H), 6.72 (s, 1H), 6.53-6.50 (dd, 1H), 6.43 (br, 1H), 6.22 (br, 1H), 4.47 (s, 4H), 4.24-4.21 (t, 2H), 4.14-4.12 (d, 2H), 4.08 (br, 2H), 3.72 (s, 3H), 3.67-3.63 (m, 2H), 3.31 (s, 3H), 3.11-3.06 (m, 2H), 2.70-2.68 (d, 2H), 2.59-2.57 (t, 2H), 2.34 (s, 3H), 2.27-2.21 (m, 1H), 2.10-2.07 (t, 2H), 1.83-1.79 (m, 2H), 1.77-1.66 (m, 5H), 1.56-1.54 (m, 4H), 1.36-1.27 (m, 2H), 1.11-1.05 (m, 4H), 1.03-0.95 (m, 5H), 0.74 (s, 6H), 0.53-0.48 (m, 1H), 0.34-0.26 (m, 2H), 0.18-0.14 (m, 1H).

### Example 212

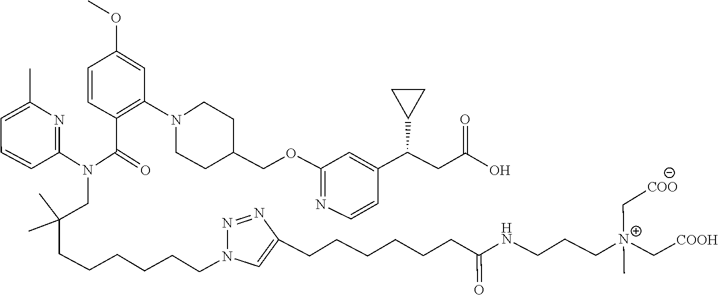

MS (ESI) m/z 1066.4 [M+H]⁺. ¹H NMR (400 MHZ, DMSO-d₆): δ 8.05-8.04 (d, 1H), 7.91 (t, 1H), 7.80 (s, 1H), 7.29-7.25 (m, 1H), 7.11-7.09 (d, 1H), 6.93-6.91 (dd, 1H), 6.85-6.83 (d, 1H), 6.72 (s, 1H), 6.53-6.50 (dd, 1H), 6.43 (br, 1H), 6.22 (br, 1H), 4.46 (s, 4H), 4.24-4.21 (t, 2H), 4.14-4.12 (d, 2H), 4.08 (br, 2H), 3.69 (s, 3H), 3.67-3.63 (m, 2H), 3.31 (s, 3H), 3.11-3.06 (m, 2H), 2.69-2.68 (d, 2H), 2.60-2.56 (t, 2H), 2.34 (s, 3H), 2.27-2.23 (m, 1H), 2.02-1.99 (t, 2H), 1.83-1.79 (m, 2H), 1.77-1.66 (m, 5H), 1.58-1.45 (m, 4H), 1.36-1.27 (m, 2H), 1.28-1.25 (m, 4H), 1.11-1.05 (m, 4H), 1.03-0.95 (m, 5H), 0.74 (s, 6H), 0.53-0.48 (m, 1H), 0.34-0.26 (m, 2H), 0.18-0.14 (m, 1H).

### Example 213

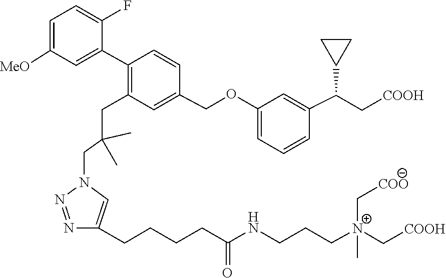

MS (ESI) m/z 844.3 [M+H]⁺.

### Example 214

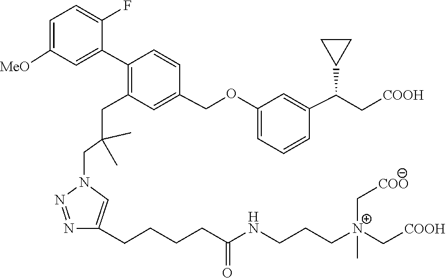

MS (ESI) m/z 872.3 [M+H]⁺. ¹H NMR (400 MHZ, DMSO-d₆): δ 7.89 (br, NH, 1H), 7.63 (s, 1H), 7.44 (s, 1H), 7.42-7.40 (d, 1H), 7.23-7.12 (m, 3H), 6.95-6.91 (m, 2H), 6.86-6.82 (m, 2H), 6.79-6.77 (m, 1H), 5.14 (s, 2H), 4.46 (s, 4H), 3.91 (s, 2H), 3.74 (s, 3H), 3.67-3.62 (m, 2H), 3.30 (s, 3H), 3.11-3.06 (m, 2H), 2.73-2.50 (m, 6H), 2.28-2.22 (m, 1H), 2.05-2.01 (m, 2H), 1.85-1.81 (m, 2H), 1.56-1.47 (m, 4H), 1.27-1.23 (m, 4H), 1.02-0.97 (m, 1H), 0.52 (br, 7H), 0.33-0.23 (m, 2H), 0.12-0.10 (m, 1H).

### Example 215

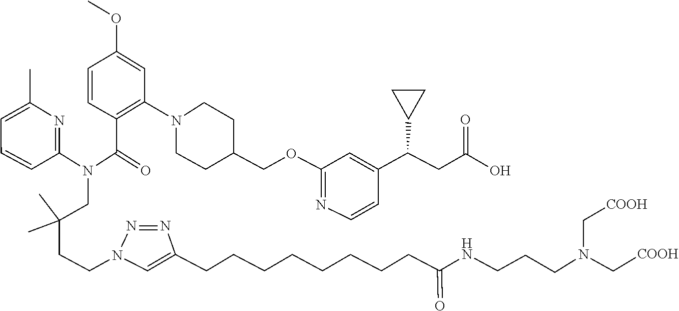 [SMILES: COc1ccc(C(=O)N(CC(C)(C)CCn2cc(CCCCCCCCC(=O)NCCCN(CC(=O)O)CC(=O)O)nn2)c2cccc(C)n2)c(N2CCC(COc3cc([C@@H](CC(=O)O)C4CC4)ccn3)CC2)c1]

MS (ESI) m/z 1024.4 [M+H]⁺. ¹H NMR (400 MHZ, DMSO-d₆): δ 8.05-8.03 (d, 1H), 7.85 (t, NH, 1H), 7.68 (s, 1H), 7.29-7.26 (br, 1H), 7.16-7.14 (d, 1H), 6.93-6.91 (dd, 1H), 6.86-6.84 (d, 1H), 6.72 (s, 1H), 6.53-6.51 (dd, 1H), 6.47 (br, 1H), 6.21 (s, 1H), 4.35-4.31 (t, 2H), 4.19 (br, 2H), 4.13-4.11 (d, 2H), 4.08 (s, 4H), 3.70 (s, 3H), 3.19-3.15 (m, B, 2H), 3.08-3.04 (m, 2H), 2.69-2.67 (m, 2H), 2.58-2.54 (t, 2H), 2.35 (s, 3H), 2.26-2.22 (m, 1H), 2.05-2.01 (t, 2H), 1.77-1.72 (m, 3H), 1.68-1.65 (m, 4H), 1.56-1.53 (m, 2H), 1.50-1.45 (m, 2H), 1.33-1.23 (br, 10H), 1.00-0.98 (m, 1H), 0.78 (s, 6H), 0.51-0.49 (m, 1H), 0.35-0.25 (m, 2H), 0.23-0.18 (m, 1H).

### Example 216

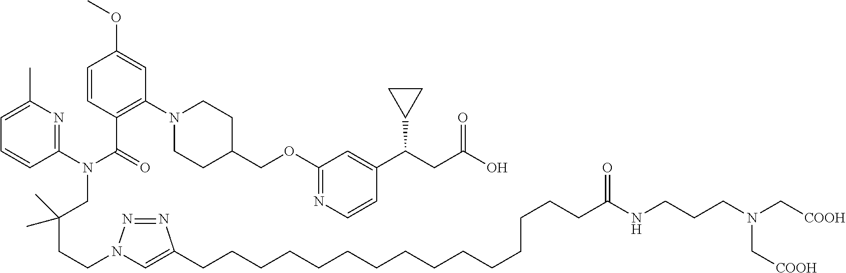 [SMILES: COc1ccc(C(=O)N(CC(C)(C)CCn2cc(CCCCCCCCCCCCCCCC(=O)NCCCN(CC(=O)O)CC(=O)O)nn2)c2cccc(C)n2)c(N2CCC(COc3cc([C@@H](CC(=O)O)C4CC4)ccn3)CC2)c1]

MS (ESI) m/z 1122.4 [M+H]⁺. ¹H NMR (400 MHZ, DMSO-d₆): δ 8.05-8.03 (d, 1H), 7.80 (t, NH, 1H), 7.67 (s, 1H), 7.29-7.26 (br, 1H), 7.16-7.14 (d, 1H), 6.92-6.91 (dd, 1H), 6.86-6.84 (d, 1H), 6.71 (s, 1H), 6.54-6.51 (dd, 1H), 6.47 (br, 1H), 6.21 (s, 1H), 4.32-4.28 (t, 2H), 4.18 (br, 2H), 4.13-4.11 (d, 2H), 3.76 (br, 4H), 3.69 (s, 3H), 3.08-3.04 (m, B, 2H), 2.91 (br, 2H), 2.69-2.67 (m, 2H), 2.58-2.54 (t, 2H), 2.36 (s, 3H), 2.27-2.20 (m, 1H), 2.05-2.01 (t, 2H), 1.76-1.65 (m, 7H), 1.57-1.53 (m, 2H), 1.48-1.45 (m, 2H), 1.33-1.23 (br, 24H), 1.01-0.97 (m, 1H), 0.79 (s, 6H), 0.52-0.50 (m, 1H), 0.36-0.27 (m, 2H), 0.23-0.18 (m, 1H).

### Example 217

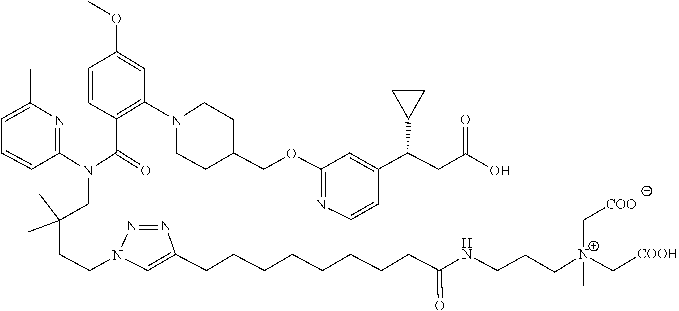

MS (ESI) m/z 1038.4 [M+H]⁺. ¹H NMR (400 MHZ, DMSO-d₆): δ 8.05-8.04 (d, 1H), 7.88 (t, NH, 1H), 7.69 (s, 1H), 7.30-7.26 (br, 1H), 7.16-7.14 (d, 1H), 6.93-6.91 (d, 1H), 6.86-6.84 (d, 1H), 6.72 (s, 1H), 6.54-6.51 (dd, 1H), 6.46-6.44 (br, 1H), 6.21 (s, 1H), 4.46 (s, 4H), 4.33-4.31 (m, 2H), 4.19 (br, 2H), 4.13-4.12 (d, 2H), 3.69 (s, 3H), 3.67-3.61 (m, B, 2H), 3.31 (s, 3H), 3.10-3.06 (m, 2H), 2.69-2.67 (m, 2H), 2.58-2.55 (m, 2H), 2.36 (s, 3H), 2.27-2.21 (m, 1H), 2.06-2.02 (t, 2H), 1.85-1.73 (m, 3H), 1.73-1.42 (m, 10H), 1.36-1.20 (br, 8H), 1.02-0.98 (m, 1H), 0.78 (s, 6H), 0.52-0.50 (m, 1H), 0.32-0.25 (m, 2H), 0.23-0.18 (m, 1H).

### Example 218

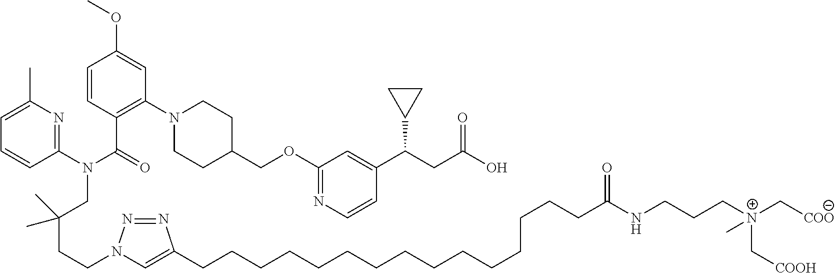

MS (ESI) m/z 1136.5 [M+H]⁺. ¹H NMR (400 MHZ, DMSO-d₆): δ 8.05-8.04 (d, 1H), 7.88 (t, NH, 1H), 7.68 (s, 1H), 7.30-7.26 (br, 1H), 7.16-7.14 (d, 1H), 6.93-6.91 (d, 1H), 6.86-6.84 (d, 1H), 6.72 (s, 1H), 6.54-6.51 (d, 1H), 6.47 (br, 1H), 6.22 (s, 1H), 4.46 (s, 4H), 4.33-4.25 (m, 2H), 4.19 (br, 2H), 4.13-4.12 (d, 2H), 3.69 (s, 3H), 3.67-3.63 (m, B, 2H), 3.31 (s, 3H), 3.09-3.07 (m, 2H), 2.69-2.67 (m, 2H), 2.58-2.54 (m, 2H), 2.36 (s, 3H), 2.27-2.21 (m, 1H), 2.05-2.02 (t, 2H), 1.85-1.47 (m, 13H), 1.36-1.20 (br, 22H), 1.02-0.98 (m, 1H), 0.78 (s, 6H), 0.52-0.50 (m, 1H), 0.32-0.25 (m, 2H), 0.23-0.18 (m, 1H).

### Example 219

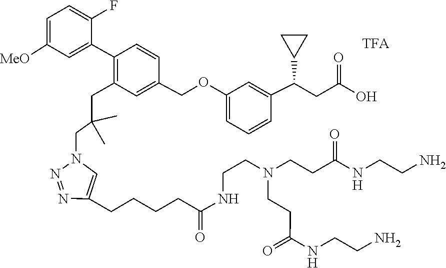 [SMILES: COc1ccc(F)c(-c2ccc(COc3cccc([C@@H](CC(=O)O)C4CC4)c3)cc2CC(C)(C)Cn2cc(CCCCC(=O)NCCN(CCC(=O)NCCN)CCC(=O)NCCN)nn2)c1]

MS (ESI) m/z 928.3 [M+H]⁺. ¹H NMR (400 MHZ, DMSO-d₆): δ 8.42 (t, NH, 2H), 8.19 (t, NH, 1H), 7.89 (br, 6H), 7.64 (s, 1H), 7.45 (s, 1H), 7.42-7.40 (d, 1H), 7.24-7.13 (m, 3H), 6.96-6.92 (m, 2H), 6.87-6.83 (m, 2H), 6.80-6.77 (m, 1H), 5.14 (s, 2H), 3.99 (s, 2H), 3.74 (s, 3H), 3.43-3.35 (m, 6H), 3.33-3.29 (m, 4H), 3.20-3.16 (m, 2H), 2.90-2.86 (m, 4H), 2.74-2.51 (m, 10H), 2.27-2.23 (m, 1H), 2.16-2.12 (t, 2H), 1.56-1.52 (m, 4H), 1.01-0.97 (m, 1H), 0.52 (br, 7H), 0.31-0.23 (m, 2H), 0.12-0.10 (m, 1H).

### Example 220

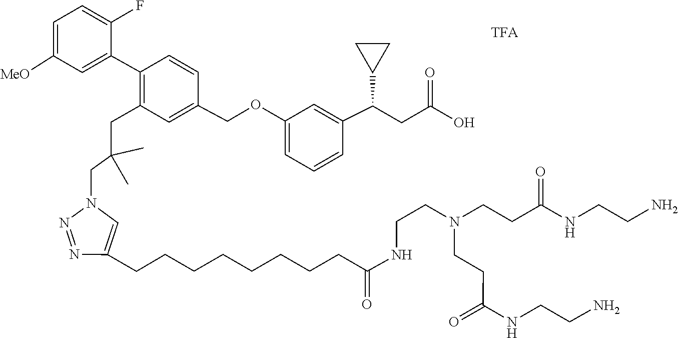 [SMILES: COc1ccc(F)c(-c2ccc(COc3cccc([C@@H](CC(=O)O)C4CC4)c3)cc2CC(C)(C)Cn2cc(CCCCCCCCC(=O)NCCN(CCC(=O)NCCN)CCC(=O)NCCN)nn2)c1]

MS (ESI) m/z 984.4 [M+H]⁺. ¹H NMR (400 MHZ, DMSO-d₆): δ 8.41 (t, NH, 2H), 8.16 (t, NH, 1H), 7.89 (br, 6H), 7.63 (s, 1H), 7.44 (s, 1H), 7.42-7.40 (d, 1H), 7.23-7.12 (m, 3H), 6.94-6.91 (m, 2H), 6.86-6.82 (m, 2H), 6.79-6.78 (m, 1H), 5.13 (s, 2H), 3.98 (s, 2H), 3.73 (s, 3H), 3.43-3.35 (m, 6H), 3.33-3.29 (m, 4H), 3.19-3.15 (m, 2H), 2.90-2.86 (m, 4H), 2.73-2.51 (m, 10H), 2.27-2.23 (m, 1H), 2.16-2.12 (t, 2H), 1.55-1.46 (m, 4H), 1.28-1.18 (m, 8H), 1.01-0.97 (m, 1H), 0.52 (br, 7H), 0.31-0.23 (m, 2H), 0.12-0.10 (m, 1H).

### Example 221

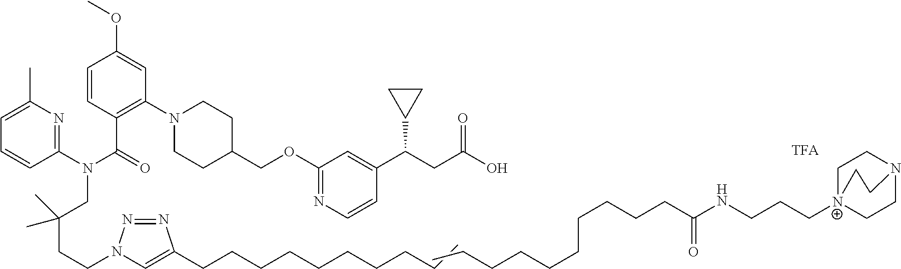 [SMILES: CCCCCCCCCCc1cn(CCC(C)(C)CN(C(=O)c2ccc(OC)cc2N2CCC(COc3cc([C@@H](CC(=O)O)C4CC4)ccn3)CC2)c2cccc(C)n2)nn1 ; CCCCCCCCCCC(=O)NCCC[N+]12CCN(CC1)CC2]

MS (ESI) m/z 1142.5 [M+H]⁺.

### Biological Example 1. Material and General Methods

IP1 accumulation assay was used to evaluate the potency of compounds. HEK293 cells stably expressing GPR40 were cultured in 5% CO₂ incubator (ThermoFisher) in maintenance media (Dulbecco's modified Eagle's medium with 4.5 g/L of glucose, 10% fetal bovine serum, 100 μg/mL Hygromycin, and Penicillin (100 U/mL)/Streptomycin (100 μg/mL)) till 100% confluency. Cells were harvested freshly, spun down at 300×g for 5 min, and resuspended in pre-warmed 1× stimulant buffer from Cisbio IP-One HTRF Detection kit (Cisbio). Cell density was adjusted to 2.0×10⁶ cells/mL. DMSO was used as blank control and AMG-1638 (CAS #: 1142214-62-7) as positive control. Compounds were prepared at 10 mM in DMSO and 5 nL of 3× serially diluted compounds (10 concentrations) were added to each well of the 384-LDV assay plate (Corning) by using ECHO 550 (Labcyte). Five μL of cells in suspension were transferred into each well by using Multidrop Combi Reagent Dispenser (ThermoFisher). Assay plate was then sealed and incubated at 37° C. for 2 hours. IP-d2 reagent and anti-IP1 reagent were prepared following the manual (Cisbio). Five μL of IP1-d2 and then 5 μL of anti-IP1 antibody was added to each well sequentially. Assay plate was incubated at room temperature for 60 min and then read at 665 nm/615 nm on an Envision plate reader (PerkinElmer). The ratio of values obtained at 665 nm and 615 nm was used for calculation of IP1 accumulation: % Effect=(Ratioₛₐₘₚₗₑ−Ratio_{blank})/(Ratio_{AMG-1638}−Ratio_{blank})×100. Dose curve was fitted and EC50 of each compound was calculated by using XLFit.

Selected compounds of the present disclosure were tested according to Biological Example 1 and the EC₅₀ values are shown in the table below.

| Example # | EC50 (nM) |
|---|---|
| 1 | 5.46 |
| 2 | 9.46 |
| 3 | 1.10 |
| 4 | 0.31 |
| 5 | 15.2 |
| 6 | 1.21 |
| 7 | 0.65 |
| 8 | >1000 |
| 9 | 640 |
| 10 | 32.7 |
| 11 | 20.3 |
| 12 | 1.12 |
| 13 | 0.41 |
| 14 | 8.80 |
| 15 | 58.4 |
| 16 | 460 |
| 17 | 152 |
| 18 | 2.25 |
| 19 | 1.66 |
| 20 | 9.76 |
| 21 | 49.4 |
| 22 | 49.0 |
| 23 | 0.96 |
| 24 | 2.04 |
| 25 | 132 |
| 26 | 75.8 |
| 27 | 5.32 |
| 28 | 4.77 |
| 29 | >1000 |
| 30 | 298 |
| 31 | 2.28 |
| 32 | 1.98 |
| 33 | 36.8 |
| 34 | 1.91 |
| 35 | 1.18 |
| 36 | 403 |
| 37 | 1000 |
| 38 | 5.42 |
| 39 | 0.83 |
| 40 | 25.3 |
| 41 | 10.1 |
| 42 | 0.29 |
| 43 | 0.21 |
| 44 | 1.74 |
| 45 | 0.68 |
| 46 | 2.03 |
| 47 | 117 |
| 48 | 4.70 |
| 49 | 19.9 |
| 50 | 8.46 |
| 51 | 318 |
| 52 | 26.0 |
| 53 | 3.18 |
| 54 | 1.71 |
| 55 | 138 |
| 56 | 1760 |
| 57 | 63.1 |
| 58 | 36.5 |
| 59 | 1255 |
| 60 | 0.44 |
| 61 | 0.23 |
| 62 | 0.61 |
| 63 | 1.23 |
| 64 | 0.68 |
| 65 | 0.28 |
| 66 | 0.49 |
| 67 | 0.38 |
| 68 | 1.00 |
| 69 | >10000 |
| 70 | 0.43 |
| 71 | 6.71 |
| 72 | 6.74 |
| 73 | 0.10 |
| 74 | 2.99 |
| 75 | 0.75 |
| 76 | 112 |
| 77 | 8400 |
| 78 | 1.40 |
| 79 | 183 |
| 80 | >5000 |
| 81 | 2.20 |
| 82 | >10000 |
| 83 | 651 |
| 84 | 0.74 |
| 85 | >1300 |
| 86 | 195 |
| 87 | >2700 |
| 88 | 22.8 |
| 89 | 2.19 |
| 90 | 2.60 |
| 91 | 1.52 |
| 92 | 212 |
| 93 | 4.58 |
| 94 | 1952 |
| 95 | >10000 |
| 96 | 1055 |
| 97 | 328 |
| 98 | 3.54 |
| 99 | 4.58 |
| 100 | 163 |
| 101 | 192 |
| 102 | 4.05 |
| 103 | 21.5 |
| 104 | >10000 |
| 105 | >10000 |
| 106 | 696 |
| 107 | 1310 |
| 108 | 312 |
| 109 | >1000 |
| 110 | 618 |
| 111 | 307 |
| 112 | 14.9 |
| 113 | 6.27 |
| 114 | 0.42 |
| 115 | 0.51 |
| 116 | 0.21 |
| 117 | 0.29 |
| 118 | 0.33 |
| 119 | 0.10 |
| 120 | 1286 |
| 121 | 5.00 |
| 122 | 1002 |
| 123 | 32.7 |
| 124 | 226 |
| 125 | 617 |
| 126 | 5.81 |
| 127 | 6.36 |
| 128 | 1.33 |
| 129 | 0.88 |
| 130 | 1.25 |
| 131 | 0.38 |
| 132 | 0.19 |
| 133 | 0.08 |
| 134 | 3600 |
| 135 | 3700 |
| 136 | 1230 |
| 137 | 2600 |
| 138 | 6.87 |
| 139 | 8.54 |
| 140 | >10000 |
| 141 | 1650 |
| 142 | 30.7 |
| 143 | >10000 |
| 144 | 5166 |
| 145 | 0.19 |
| 146 | 0.12 |
| 147 | 0.62 |
| 148 | 0.43 |
| 149 | 0.09 |
| 150 | 0.07 |
| 151 | 0.05 |
| 152 | 34.4 |
| 153 | 6.64 |
| 154 | 1.04 |
| 155 | 18.8 |
| 156 | 2.53 |
| 157 | 0.54 |
| 158 | 8.97 |
| 159 | >6000 |
| 160 | 1365 |
| 161 | 2200 |
| 162 | 1252 |
| 163 | >5000 |
| 164 | 1331 |
| 165 | >10000 |
| 166 | >10000 |
| 167 | >10000 |
| 168 | >10000 |
| 169 | >10000 |
| 170 | 200 |
| 171 | >10000 |
| 172 | 5240 |
| 173 | >10000 |
| 174 | >10000 |
| 175 | >10000 |
| 176 | >10000 |
| 177 | 0.08 |
| 178 | 2028 |
| 179 | 50.0 |
| 180 | >10000 |
| 181 | 16.1 |
| 182 | >10000 |
| 183 | 0.16 |
| 184 | 0.16 |
| 185 | 0.05 |
| 186 | 0.03 |
| 187 | 1.34 |
| 188 | 1.53 |
| 189 | 0.05 |
| 190 | 0.05 |
| 191 | 204 |
| 192 | 250 |
| 193 | 413 |
| 194 | 114 |
| 195 | >10000 |
| 196 | 9872 |
| 197 | 84.3 |
| 198 | 0.17 |
| 199 | >10000 |
| 200 | >10000 |
| 201 | 44.6 |
| 202 | 0.16 |
| 203 | 11.9 |
| 204 | 7.00 |
| 205 | 0.93 |
| 206 | 0.59 |
| 207 | 20.5 |
| 208 | 5.15 |
| 209 | 1.00 |
| 210 | 0.54 |
| 211 | 66.3 |
| 212 | 143 |
| 213 | 358 |
| 214 | 631 |
| 215 | 1357 |
| 216 | 0.48 |
| 217 | 683 |
| 218 | 0.44 |
| 219 | >10000 |
| 220 | >10000 |
| 221 | 0.98 |

The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus, all individual species are individually considered separate aspects of the invention. If aspects of the invention are described as “comprising” a feature, embodiments also are contemplated “consisting of” or “consisting essentially of” the feature.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

What is claimed is:

1. A compound of Formula I, or a pharmaceutically acceptable salt or ester thereof: 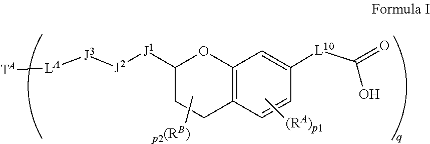
wherein:
L¹⁰ is an alkylene (e.g., a C₁₋₆ alkylene), optionally substituted with 1-3 substituents independently selected from halogen, optionally substituted C₁₋₆ alkyl, optionally substituted C₂₋₆ alkenyl, optionally substituted C₂₋₆ alkynyl, optionally substituted C₁₋₆ heteroalkyl, optionally substituted C₃₋₆ cycloalkyl, optionally substituted C₁₋₆ alkoxy, optionally substituted C₃₋₆ cycloalkoxy, optionally substituted heterocyclyl, optionally substituted aryl, and optionally substituted heteroaryl, or two substituents are joined to form an optionally substituted ring structure;
R^{A} at each occurrence is independently halogen, CN, optionally substituted C₁₋₆ alkyl, optionally substituted C₃₋₆ cycloalkyl, optionally substituted C₁₋₆ alkoxy, or optionally substituted C₃₋₆ cycloalkoxy, or two R^{A} are joined to form an optionally substituted ring structure; p1 is 0, 1, or 2;
R^{B} at each occurrence is independently halogen, hydroxyl, amino, substituted amino, optionally substituted C₁₋₆ alkyl, optionally substituted C₃₋₆ cycloalkyl, optionally substituted C₁₋₆ alkoxy, or optionally substituted C₃₋₆ cycloalkoxy, or two R^{B} are joined to form an optionally substituted ring structure; p2 is 0, 1, 2, 3, or 4;
J¹ is a bond, an optionally substituted aryl or heteroaryl ring, —C₁₋₆alkylene-N(R¹⁰⁰)—, 3-14 membered optionally substituted heterocyclylene containing at least one ring nitrogen atom, or —C₁₋₆alkylene-(3-14 membered optionally substituted heterocyclylene containing at least one ring nitrogen atom)-, wherein R¹⁰⁰ is hydrogen, optionally substituted alkyl, or optionally substituted cycloalkyl;
J² is a bond or an alkylene, optionally substituted with 1-3 substituents independently selected from halogen, optionally substituted C₁₋₆ alkyl, optionally substituted C₂₋₆ alkenyl, optionally substituted C₂₋₆ alkynyl, optionally substituted C₁₋₆ heteroalkyl, optionally substituted C₃₋₆ cycloalkyl, optionally substituted C₁₋₆ alkoxy, optionally substituted C₃₋₆ cycloalkoxy, or two substituents are joined to form an optionally substituted ring structure;
J³ is an optionally substituted cycloalkyl, heterocyclyl, aryl or heteroaryl ring, and
wherein:
q is an integer of 1-10, preferably, 1 or 2,
T^{A} is a hydrophilic group having a terminal atom(s) selected from N, O, S, or C, which is covalently bonded with a first end atom of L^{A}, wherein (1) when the terminal atom(s) is N of a basic amine group, then the corresponding compound T^{A}-(C(O)—CH₃)_{q} has a cLogP of less than 1, wherein the —C(O)—CH₃ is bonded with the terminal N atom(s); (2) when the terminal atom(s) is C of a C(O) group, then the corresponding compound T^{A}-(OH)_{q} has a cLogP of less than 1, wherein the —OH is bonded with the terminal C atom(s); (3) when the terminal atom(s) is S of a SO₂ group, then the corresponding compound T^{A}-(OH)_{q} has a cLogP of less than 1, wherein the —OH is bonded with the terminal S atom(s); or (4) when (1)-(3) do not apply, then the corresponding compound T^{A}-H_{q} has a cLogP of less than 1; and
L^{A} is a linker characterized in that the maximum length between the two end atoms of L^{A} is at least the maximum length between the two end carbon atoms of —(CH₂)₁₀—, wherein (1) when both end atoms of L^{A} are C of a C(O) or S of a SO₂ group, then the corresponding compound HO-L^{A}-OH has a cLogP of at least 3, wherein each —OH is bonded with the end C(O) or SO₂ group; (2) when only one end atom of L^{A} is C of a C(O) or S of a SO₂ group, then the corresponding compound H-L^{A}-OH has a cLogP of at least 3, wherein the —OH is bonded with the C(O) or SO₂ group; or (3) when neither (1) and (2) applies, then the corresponding compound H-L^{A}-H has a cLogP of at least 3.

2. The compound of claim 1, or a pharmaceutically acceptable salt or ester thereof, wherein q is 1 and the compound has a Formula I-1:

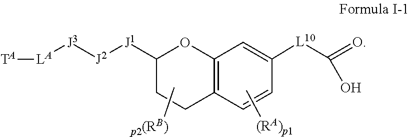

3. The compound of claim 1 or 2, or a pharmaceutically acceptable salt or ester thereof, wherein p1 is 0.

4. The compound of claim 1 or 2, or a pharmaceutically acceptable salt or ester thereof, wherein p1 is 1, and R^{A} is F, Cl, CN, C₁₋₄ alkyl optionally substituted with 1-3 fluorine, or C₁₋₄ alkoxy optionally substituted with 1-3 fluorine.

5. The compound of any of claims 1-4, or a pharmaceutically acceptable salt or ester thereof, wherein p2 is 0.

6. The compound of any of claims 1-4, or a pharmaceutically acceptable salt or ester thereof, wherein p2 is 1 or 2, and R^{B} at each occurrence is independently F, OH, NH₂, NH(C₁₋₄ alkyl), N(C₁₋₄ alkyl)(C₁₋₄ alkyl), C₁₋₄ alkyl optionally substituted with 1-3 fluorine, or C₁₋₄ alkoxy optionally substituted with 1-3 fluorine.

7. The compound of claim 1 or 2, or a pharmaceutically acceptable salt or ester thereof, which has a Formula I-1-A: 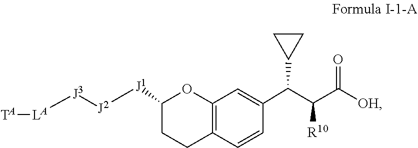
wherein R¹⁰ is hydrogen or C₁₋₄ alkyl (preferably methyl).

8. The compound of any of claims 1-7, or a pharmaceutically acceptable salt or ester thereof, wherein J¹ is a 4-12 membered optionally substituted heterocyclic ring having one or two ring nitrogen atoms.

9. The compound of claim 8, or a pharmaceutically acceptable salt or ester thereof, wherein J¹ is a 4-8 membered optionally substituted monocyclic saturated heterocyclic ring having one or two ring heteroatoms independently selected from S, O, and N, provided at least one of the ring heteroatom is nitrogen.

10. The compound of claim 9, or a pharmaceutically acceptable salt or ester thereof, wherein J¹ is selected from: 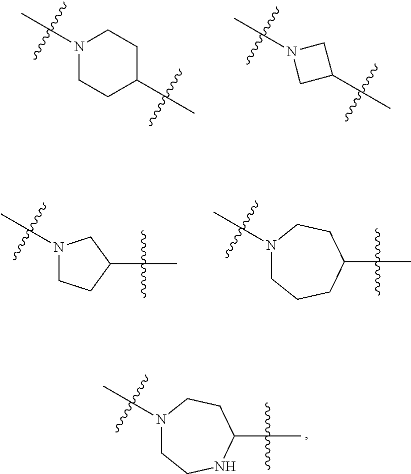 [SMILES: *C1CCN(*)CC1 ; *C1CN(*)C1 ; *C1CCN(*)C1 ; *C1CCCN(*)CC1 ; *C1CCN(*)CCN1]
each of which is optionally substituted with 1-2 substituents independently selected from F, OH, NH₂, NH(C₁₋₄ alkyl), N(C₁₋₄ alkyl)(C₁₋₄ alkyl), C₁₋₄ alkyl optionally substituted with 1-3 fluorine, or C₁₋₄ alkoxy optionally substituted with 1-3 fluorine.

11. The compound of claim 8, or a pharmaceutically acceptable salt or ester thereof, wherein J¹ is bicyclic or polycyclic 6-12 membered optionally substituted saturated heterocyclic ring having one or two ring heteroatoms independently selected from S, O, and N, provided at least one of the ring heteroatom is nitrogen.

12. The compound of any of claims 1-11, or a pharmaceutically acceptable salt or ester thereof, wherein J² is a straight chain or branched C₁₋₄ alkylene, optionally substituted with 1-3 fluorine.

13. The compound of any of claims 1-11, or a pharmaceutically acceptable salt or ester thereof, wherein J² is CH₂ or —CH(CH₃)—.

14. The compound of any of claims 1-13, or a pharmaceutically acceptable salt or ester thereof, wherein J³ is an aryl or heteroaryl ring, each of which is unsubstituted or substituted with one or more (e.g., 1, 2, or 3) substituents independently selected from 1) halogen, CN, —CF₃, OH, amino, substituted amino, ester, amide, carbonate, or carbamate; and 2) C₁₋₆ alkyl, C₂₋₆ alkenyl, C₂₋₆ alkynyl, C₁₋₆ heteroalkyl, C₃₋₆ cycloalkyl, C₁₋₆ alkyl alkoxy, C₃₋₆ cycloalkoxy, aryl, heteroaryl, 3-8 membered heterocycloalkyl having one or two ring heteroatoms independently selected from N, O, and S, wherein each of which is optionally substituted with one or more (e.g., 1, 2, or 3) substituents independently selected from F, —OH, protected hydroxyl, oxo (as applicable), NH₂, protected amino, NH(C₁₋₄ alkyl) or a protected derivative thereof, N(C₁₋₄ alkyl((C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2, or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy.

15. The compound of any of claims 1-14, or a pharmaceutically acceptable salt or ester thereof, wherein J³ is a phenyl ring, which is substituted with 1-3 substituents independently selected from F, Cl, CN, OH, C₁₋₆ alkyl, C₁₋₆ alkyl heteroalkyl, C₃₋₆ cycloalkyl, C₁₋₆ alkyl alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), C₁₋₄ alkyl optionally substituted with F.

16. The compound of any of claims 1-14, or a pharmaceutically acceptable salt or ester thereof, wherein J³ is a 5-10 membered monocyclic or bicyclic heteroaryl ring, which is substituted with 1-3 substituents independently selected from F, Cl, CN, OH, C₁₋₆ alkyl, C₁₋₆ alkyl heteroalkyl, C₃₋₆ cycloalkyl, C₁₋₆ alkyl alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), C₁₋₄ alkyl optionally substituted with F.

17. The compound of any of claims 1-14, or a pharmaceutically acceptable salt or ester thereof, wherein J³ is selected from: 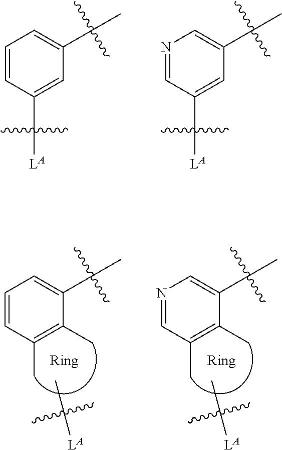
wherein: Ring represents an aromatic or non-aromatic ring structure,
wherein each of the phenyl, pyridyl, or fused ring structure is optionally substituted with 1-3 substituents independently selected from F, Cl, CN, OH, C₁₋₆ alkyl, C₁₋₆ alkyl heteroalkyl, C₃₋₆ cycloalkyl, C₁₋₆ alkyl alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), C₁₋₄ alkyl optionally substituted with F.

18. The compound of any of claims 1-14, or a pharmaceutically acceptable salt or ester thereof, wherein J³ is selected from: 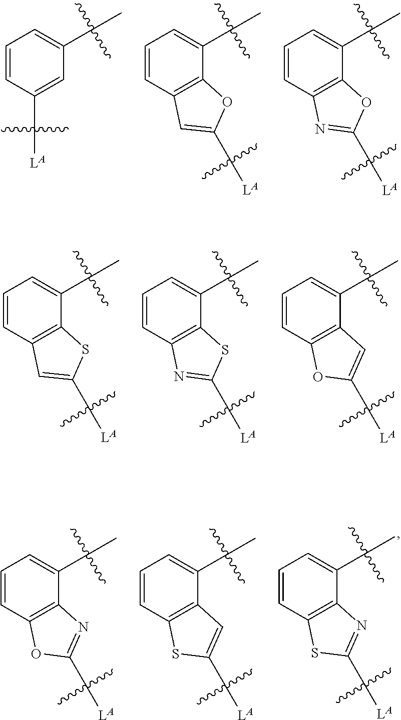
wherein each of which is optionally substituted with 1-3 substituents independently selected from F, Cl, CN, OH, C₁₋₆ alkyl, C₁₋₆ alkyl heteroalkyl, C₃₋₆ cycloalkyl, C₁₋₆ alkyl alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), C₁₋₄ alkyl optionally substituted with F.

19. The compound of claim 18, or a pharmaceutically acceptable salt or ester thereof, wherein J³ is selected from: 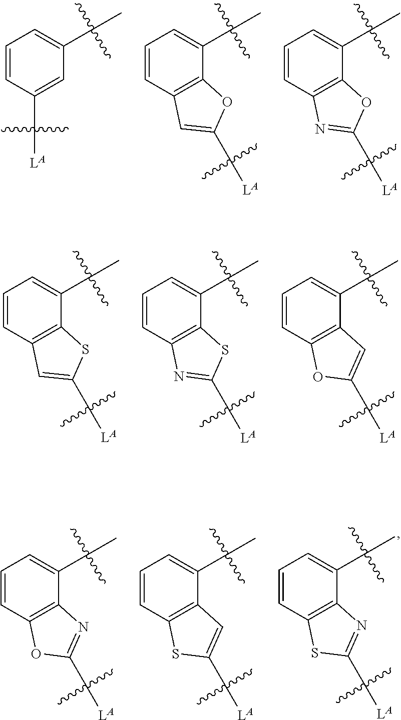
wherein each of which is optionally substituted with 1-3 substituents independently selected from F, Cl, CN, OH, C₁₋₆ alkyl optionally substituted with F (e.g., CF₃), cyclopropyl, cyclobutyl, C₁₋₆ alkyl alkoxy optionally substituted with F (e.g., —O—CF₃), or C₃₋₆ cycloalkoxy.

20. The compound of claim 1, or a pharmaceutically acceptable salt or ester thereof, wherein q is 1 and the compound has a Formula I-1-A-1, I-1-A-2, I-1-A-3, I-1-A-4, or I-1-A-5: 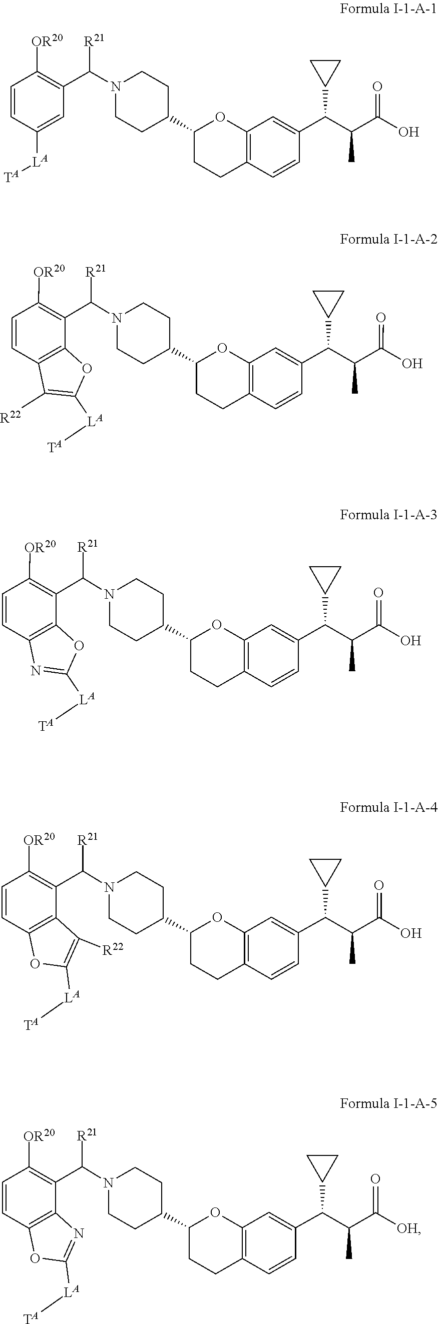
wherein:
R²⁰ is C₁₋₆ alkyl or fluorine substituted C₁₋₆ alkyl, R²¹ is hydrogen or C₁₋₆ alkyl, and R²² is hydrogen, halogen, CN, C₁₋₆ alkyl or fluorine substituted C₁₋₆ alkyl or a C₃₋₆ cycloalkyl.

21. The compound of claim 1, or a pharmaceutically acceptable salt or ester thereof, wherein q is 1 and the compound has a Formula I-1-A-6, I-1-A-7, I-1-A-8, I-1-A-9, or I-1-A-10: 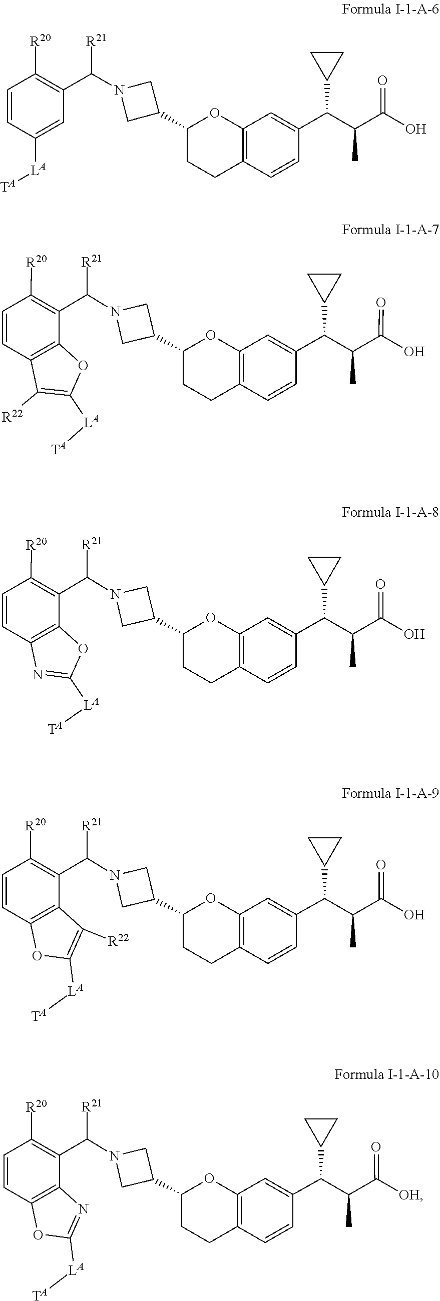
wherein:
R²⁰ is C₁₋₆ alkyl or fluorine substituted C₁₋₆ alkyl, R²¹ is hydrogen or C₁₋₆ alkyl, and R²² is hydrogen, halogen, CN, C₁₋₆ alkyl or fluorine substituted C₁₋₆ alkyl or a C₃₋₆ cycloalkyl.

22. The compound of any of claims 1-21, or a pharmaceutically acceptable salt or ester thereof, wherein L^{A} is characterized in that the maximum length between the two end atoms of L^{A} is at least that between the two end carbon atoms of —(CH₂)₁₂—, preferably, at least that of —(CH₂)₁₄—, more preferably, at least that of —(CH₂)₁₆—.

23. The compound of any of claims 1-21, or a pharmaceutically acceptable salt or ester thereof, wherein L^{A} is characterized in that the maximum length between the two end atoms of L^{A} is between (i) the maximum length between the two end carbon atoms of —(CH₂)₁₂— and (ii) the maximum length between the two end carbon atoms of —(CH₂)₅₀—.

24. The compound of any of claims 1-23, or a pharmaceutically acceptable salt or ester thereof, wherein only one end atom of L^{A} is C of a C(O) or S of a SO₂ group.

25. The compound of claim 24, or a pharmaceutically acceptable salt or ester thereof, wherein L^{A} is characterized in that the corresponding compound H-L^{A}-OH has a cLogP of at least 4, wherein the —OH is bonded with the end C(O) or SO₂ group.

26. The compound of claim 24, or a pharmaceutically acceptable salt or ester thereof, wherein L^{A} is characterized in that the corresponding compound H-L^{A}-OH has a cLogP of between 3-15, wherein the —OH is bonded with the end C(O) or SO₂ group.

27. The compound of any of claims 1-26, or a pharmaceutically acceptable salt or ester thereof, wherein (1) the terminal atom(s) is N of a basic amine group, and T^{A} is characterized in that the corresponding compound T^{A}-(C(O)—CH₃)_{q} has a cLogP of less than 0 (e.g., less than −1, less than −2, less than −3, less than −3.5, less than −4, or even lower); (2) the terminal atom(s) is C of a C(O) group, and T^{A} is characterized in that the corresponding compound T^{A}-(OH) q has a cLogP of less than 0 (e.g., less than −1, less than −2, less than −3, less than −3.5, less than −4, or even lower); (3) the terminal atom(s) is S in a SO₂ group, and T^{A} is characterized in that the corresponding compound T^{A}-(OH)_{q} has a cLogP of less than 0 (e.g., less than −1, less than −2, less than −3, less than −3.5, less than −4, or even lower); or (4) the terminal atom(s) is not N of a basic amine group, C of a C(O) group, or S in a SO₂ group, and T^{A} is characterized in that the corresponding compound T^{A}-H_{q} has a cLogP of less than 0 (e.g., less than −1, less than −2, less than −3, less than −3.5, less than −4, or even lower).

28. The compound of any of claims 1-26, or a pharmaceutically acceptable salt or ester thereof, wherein T^{A} is T^{A1}, T^{A1}-L^{B}-, T^{A1}-L^{B}-(heteroalkylene), or T^{A1}-L^{B}-(heteroalkylene)-L^{B}-,
wherein L^{B} at each occurrence is independently —SO₂—, —C(═O)—, or a moiety selected from: 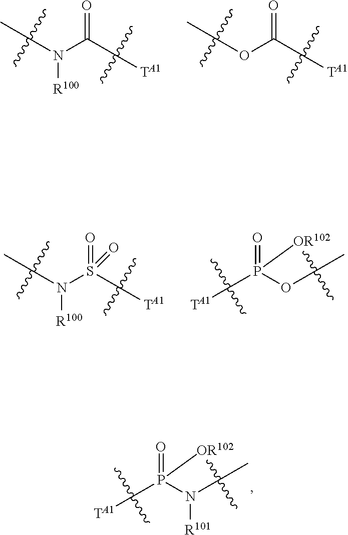
wherein R¹⁰⁰, R¹⁰¹ and R¹⁰² at each occurrence is independently hydrogen, optionally substituted alkyl, or optionally substituted cycloalkyl,
wherein T^{A1} is characterized as having a structure of: 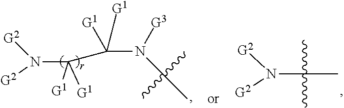
wherein G¹ at each occurrence is independently hydrogen, halogen, optionally substituted C₁₋₄ alkyl, or optionally substituted C₁₋₄ heteroalkyl (such as optionally substituted C₁₋₄ alkoxy), or two gem G¹ group can join to form an —O, ═NH, or ═N(C₁₋₄ alkyl), or two or more G¹ groups can join to form a ring structure,
the integer r is 1-10, such as 1 or 2,
G² at each occurrence is independently hydrogen, G¹⁰, C(O)-G¹⁰, SO₂G¹⁰, C(O)—NH-G¹⁰, or SO₂NHG¹⁰, C(O)—O-G¹⁰, or SO₂OG¹⁰, wherein G¹⁰ at each occurrence is independently an optionally substituted alkyl, an optionally substituted heteroalkyl, or an optionally substituted 3-10 membered ring structure, which can be carbocyclic, heterocyclic, aromatic, heteroaromatic, or a combination thereof,
G³ is hydrogen or an optionally substituted alkyl (e.g., C₁₋₄ alkyl);
or two G² or one G² and one G¹ or one G¹ and G³ can be joined to form a ring structure such as a 3-10 membered ring structure,
wherein the fragment 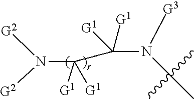
is further characterized in that (i) when the CG¹G¹ group next to the NG³ is not C(═O), the corresponding compound 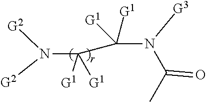
has a cLogP less than 1, preferably, less than 0 (e.g., less than −1, less than −2, less than −3, less than −3.5, less than −4, or even lower), or (ii) when the CG¹G¹ group next to the NG³ is C(═O), the corresponding compound 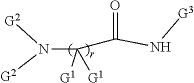
has a cLogP less than 1, preferably, less than 0 (e.g., less than −1, less than −2, less than −3, less than −3.5, less than −4, or even lower), and
wherein the fragment 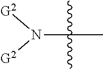
is further characterized in that (i) when the nitrogen is a basic nitrogen, the corresponding compound (G²)₂N—C(O)—CH₃ has a cLogP less than 1, preferably, less than 0 (e.g., less than −1, less than −2, less than −3, less than −3.5, less than −4, or even lower), or (ii) when the nitrogen is a nonbasic nitrogen, the corresponding compound (G²)₂N—H has a cLogP less than 1, preferably, less than 0 (e.g., less than −1, less than −2, less than −3, less than −3.5, less than −4, or even lower).

29. The compound of any of claims 1-26, or a pharmaceutically acceptable salt or ester thereof, wherein T^{A} is T^{A1}, T^{A1}-L^{B}-, T^{A1}-L^{B}-(heteroalkylene), or T^{A1}-L^{B}-(heteroalkylene)-L^{B}-,
wherein L^{B} at each occurrence is independently —SO₂—, —C(═O)—, or a moiety selected from: 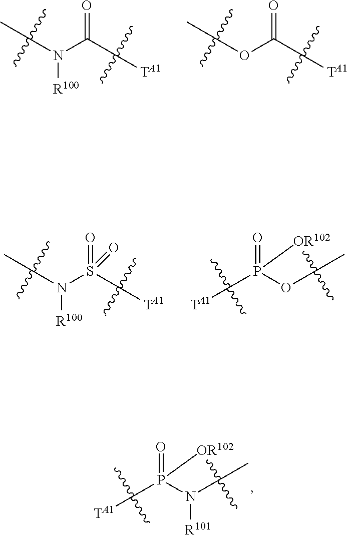
wherein R¹⁰⁰, R¹⁰¹ and R¹⁰² at each occurrence is independently hydrogen, optionally substituted alkyl, or optionally substituted cycloalkyl,
wherein T^{A1} is characterized as having a structure of: 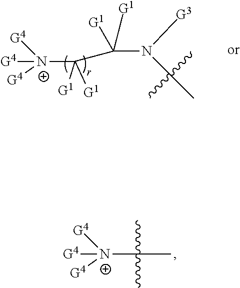
charge balanced with a counterion (e.g., described herein) as necessary,
wherein G¹ at each occurrence is independently hydrogen, halogen, optionally substituted C₁₋₄ alkyl, or optionally substituted C₁₋₄ heteroalkyl (such as optionally substituted C₁₋₄ alkoxy), or two gem G¹ group can join to form an ═O, ═NH, or ═N(C₁₋₄ alkyl), or two or more G¹ groups can join to form a ring structure,
the integer r is 1-10, such as 1 or 2,
G³ is hydrogen or an optionally substituted alkyl (e.g., C₁₋₄ alkyl);
G⁴ at each occurrence is independently hydrogen or G¹⁰, wherein G¹⁰ at each occurrence is independently an optionally substituted alkyl, an optionally substituted heteroalkyl, or an optionally substituted 3-10 membered ring structure, which can be carbocyclic, heterocyclic, aromatic, heteroaromatic, or a combination thereof, or two G⁴ or one G⁴ and one G¹ or one G¹ and G³ can be joined to form an optionally substituted 3-10 membered ring structure,
wherein the fragment 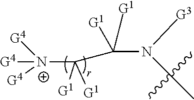
is characterized in that (i) when the CG¹G¹ group next to the NH is not C(═O), the corresponding compound 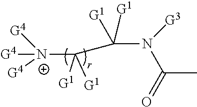
has a cLogP less than 1, preferably, less than 0 (e.g., less than −1, less than −2, less than −3, less than −3.5, less than −4, or even lower), or (ii) when the CG¹G¹ group next to the NH is C(═O), the corresponding compound 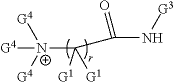
has a cLogP less than 1, preferably, less than 0 (e.g., less than −1, less than −2, less than −3, less than −3.5, less than −4, or even lower).

30. The compound of claim 29 or 30, or a pharmaceutically acceptable salt or ester thereof, wherein G³ is hydrogen and/or G¹ at each occurrence is hydrogen.

31. The compound of any of claims 1-30, or a pharmaceutically acceptable salt or ester thereof, wherein T^{A} is characterized as having a charged group (including zwitterion structures) or a group that can become charged at pH 7, such as primary amine, secondary amine, tertiary amine, quaternary amine, carboxylic acid, etc.

32. The compound of any of claims 1-31, or a pharmaceutically acceptable salt or ester thereof, wherein T^{A} is characterized as having at least two hydrogen bond donors.

33. The compound of any of claims 1-32, or a pharmaceutically acceptable salt or ester thereof, wherein T^{A} is characterized as having at least two hydrogen bond acceptors.

34. The compound of any of claims 1-33, or a pharmaceutically acceptable salt or ester thereof, wherein the covalent bond(s) between the terminal atom(s) of T^{A} and the first end atom of L^{A} is an amide bond.

35. The compound of any of claims 1-33, or a pharmaceutically acceptable salt or ester thereof, wherein the covalent bond(s) between the terminal atom(s) of T^{A} and the first end atom of L^{A} is a non-amide carbon-nitrogen bond, an ester bond, a non-ester carbon-oxygen bond, a carbon-carbon bond, or a carbon-sulfur bond.

36. The compound of any of claims 1-35, or a pharmaceutically acceptable salt or ester thereof, wherein L^{A} is (X) m, wherein X at each occurrence is independently CR₂, C(═O), —C(R)═C(R)—, 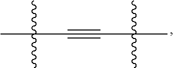 [SMILES: *C#C*]
SiR₂, O, S, SO₂, NR, [NR₂]⁺, or a ring structure, preferably 3-10 membered ring structure, wherein R at each occurrence is independently hydrogen, halogen, optionally substituted C₁₋₄ alkyl, or optionally substituted C₁₋₄ alkoxy, typically R is hydrogen or C₁₋₄ alkyl, and the integer m is at least 10, such as at least 12, at least 14, at least 16, at least 18, at least 20, at least 50, such as 12-50, 16-50, etc.

37. The compound of any of claims 1-35, or a pharmaceutically acceptable salt or ester thereof, wherein L^{A} is —(X)ₘ₋₁—C(O)—, wherein the C(O) end is bonded with T^{A}, wherein X at each occurrence is independently CR₂, C(═O), —C(R)═C(R)—, 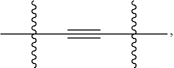 [SMILES: *C#C*]
SiR₂, O, S, SO₂, NR, [NR₂]⁺ or a ring structure, preferably 3-10 membered ring structure, provided that the end X group is not C(O) or SO₂, wherein R at each occurrence is independently hydrogen, halogen, optionally substituted C₁₋₄ alkyl, or optionally substituted C₁₋₄ alkoxy, typically R is hydrogen or C₁₋₄ alkyl, and the integer m is at least 10, such as at least 12, at least 14, at least 16, at least 18, at least 20, at least 50, such as 12-50, 16-50, etc., and the hydrophobicity of —(X)ₘ₋₁—C(O)— is characterized in that the corresponding compound H—(X)ₘ₋₁—COOH has a cLogP of at least 3, such as between 3-15, preferably, at least 4.

38. The compound of any of claims 1-35, or a pharmaceutically acceptable salt or ester thereof, wherein L^{A} is —C₁₂₋₃₀ alkylene- or —C₁₂₋₃₀ alkylene-C(O)—, wherein the —C₁₂₋₃₀ alkylene- is optionally substituted, wherein the optional substituents can optionally be joined together to form a double bond, triple bond, or a ring structure.

39. The compound of any of claims 1-35, or a pharmaceutically acceptable salt or ester thereof, wherein L^{A} is a 12-30 membered heteroalkylene or -(12-30 membered heteroalkylene)-C(O)—, wherein the 12-30 membered heteroalkylene is optionally substituted and contains 1-6 heteroatoms independently selected from O, N, and S, wherein the sulfur atom(s), if present, is optionally oxidized, wherein the optional substituents can optionally be joined together to form a double bond, triple bond, or a ring structure.

40. The compound of any of claims 1-35, or a pharmaceutically acceptable salt or ester thereof, wherein L^{A} is 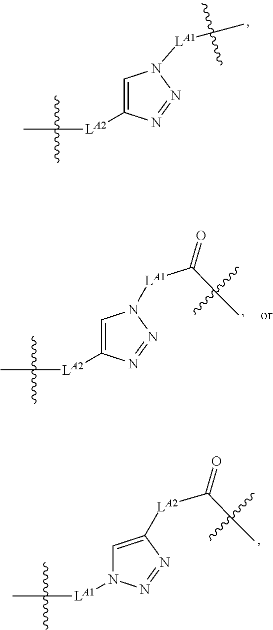
wherein L^{A1} and L^{A2} are each independently a bond, an optionally substituted —C₁₋₃₀ alkylene-, or an optionally substituted —C₁₋₃₀ heteroalkylene-containing 1-6 heteroatoms independently selected from O, N, and S, wherein the sulfur atom(s), if present, is optionally oxidized, wherein the optional substituents can optionally be joined together to form a double bond, triple bond, or a ring structure, provided that the longest chain length of L^{A} is at least that of -(CH₂)₁₂-, such as at least that of —(CH₂)₁₄—, at least that of —(CH₂)₁₆—, at least that of —(CH₂)₁₈—.

41. The compound of any of claims 1-40, or a pharmaceutically acceptable salt or ester thereof, wherein T^{A} is T^{A1} or TAL-C(O)—, wherein T^{A1} is 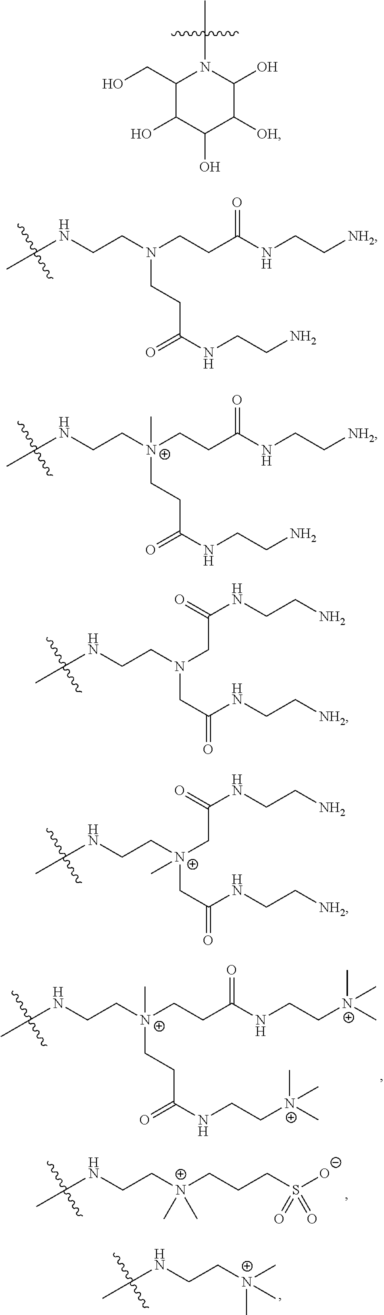 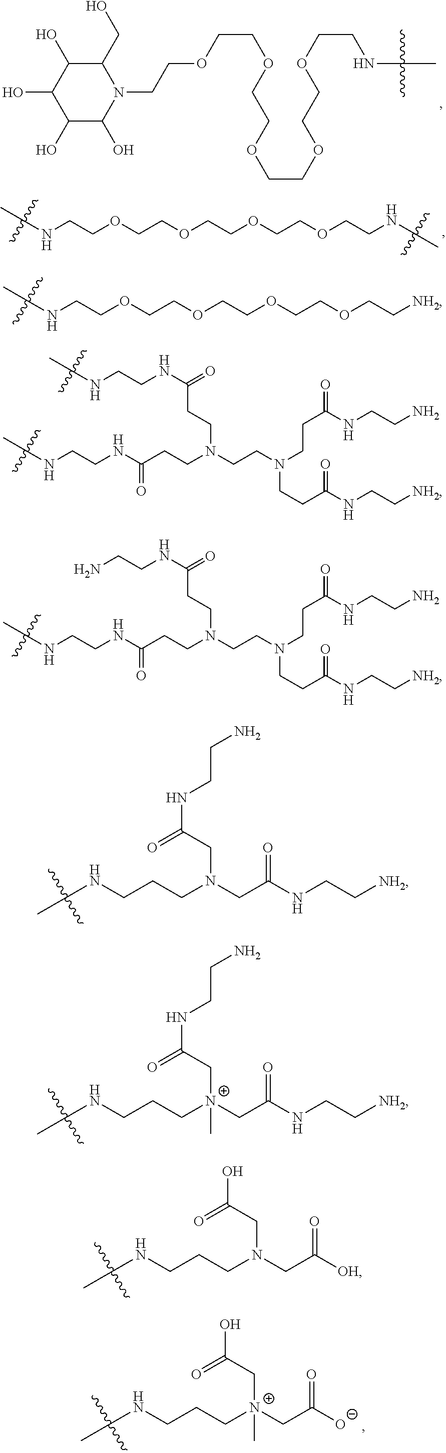 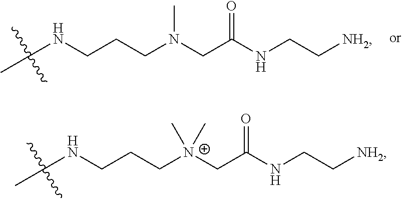
wherein T^{A} is charge balanced with a counterion (e.g., described herein) as necessary.

42. A compound of Formula II or II-B, or a pharmaceutically acceptable salt or ester thereof: 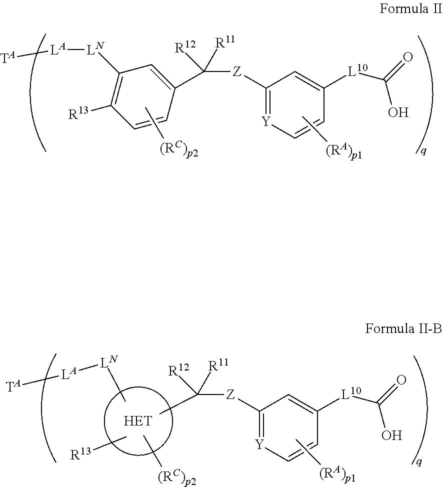
wherein:
Y is CH, CR^{A}, or N;
Z is O, S, NH, or N(C₁₋₄ alkyl);
HET ring stands for an optionally substituted heteroaryl ring (e.g., a 5 or 6-membered heteroaryl, such as a triazole ring);
R¹¹ and R¹² are each independently hydrogen or C₁₋₄ alkyl;
L^{N} is null, an optionally substituted C₁₋₆ alkylene, or an optionally substituted C₁₋₆ heteroalkylene having 1-3 heteroatoms;
L¹⁰ is an alkylene, optionally substituted with 1-3 substituents independently selected from halogen, optionally substituted C₁₋₆ alkyl, optionally substituted C₂₋₆ alkenyl, optionally substituted C₂₋₆ alkynyl, optionally substituted C₁₋₆ heteroalkyl, optionally substituted C₃₋₆ cycloalkyl, optionally substituted C₁₋₆ alkoxy, optionally substituted C₃₋₆ cycloalkoxy, optionally substituted heterocyclyl, optionally substituted aryl, and optionally substituted heteroaryl, or two substituents are joined to form an optionally substituted ring structure;
R^{A} at each occurrence is independently halogen, CN, optionally substituted C₁₋₆ alkyl, optionally substituted C₃₋₆ cycloalkyl, optionally substituted C₁₋₆ alkoxy, or optionally substituted C₃₋₆ cycloalkoxy, or two R^{A} are joined to form an optionally substituted ring structure; p1 is 0, 1, or 2;
R^{C} at each occurrence is independently halogen, CN, optionally substituted C₁₋₆ alkyl, optionally substituted C₃₋₆ cycloalkyl, optionally substituted C₁₋₆ alkoxy, or optionally substituted C₃₋₆ cycloalkoxy, or two R^{C} are joined to form an optionally substituted ring structure; p2 is 0, 1, 2, or 3;
R¹³ is hydrogen, an optionally substituted phenyl or an optionally substituted heteroaryl, and wherein:
q is an integer of 1-10, preferably, 1 or 2,
T^{A} is a hydrophilic group having a terminal atom(s) selected from N, O, S, or C, which is covalently bonded with a first end atom of L^{A}, wherein (1) when the terminal atom(s) is N of a basic amine group, then the corresponding compound T^{A}-(C(O)—CH₃)_{q} has a cLogP of less than 1, wherein the —C(O)—CH₃ is bonded with the terminal N atom(s); (2) when the terminal atom(s) is C of a C(O) group, then the corresponding compound T^{A}-(OH)_{q} has a cLogP of less than 1, wherein the —OH is bonded with the terminal C atom(s); (3) when the terminal atom(s) is S of a SO₂ group, then the corresponding compound T^{A}-(OH)_{q} has a cLogP of less than 1, wherein the —OH is bonded with the terminal S atom(s); or (4) when (1)-(3) do not apply, then the corresponding compound T^{A}-H_{q} has a cLogP of less than 1; and
L^{A} is a linker characterized in that the maximum length between the two end atoms L^{A} is at least the maximum length between the two end carbon atoms of (CH₂)₁₀, wherein (1) when both end atoms of L^{A} are C of a C(O) or S of a SO₂ group, then the corresponding compound HO-L^{A}-OH has a cLogP of at least 3, wherein each —OH is bonded with the end C(O) or SO₂ group; (2) when only one end atom of L^{A} is C of a C(O) or S of a SO₂ group, then the corresponding compound H-L^{A}-OH has a cLogP of at least 3, wherein the —OH is bonded with the end C(O) or SO₂ group; or (3) when neither (1) and (2) applies, then the corresponding compound H-L^{A}-H has a cLogP of at least 3.

43. The compound of claim 42, or a pharmaceutically acceptable salt or ester thereof, wherein q is 1 and the compound has a Formula II-1 or II-B-1:

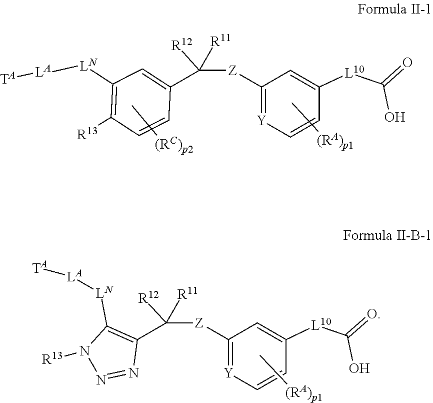

44. The compound of claim 42 or 43, or a pharmaceutically acceptable salt or ester thereof, wherein L^{N} is (i) null; (ii) a branched or straight chained C₁₋₆ alkyl alkylene, such as 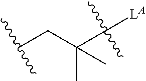
(L^{A} is shown to show direction of connection); or (iii) a branched or straight chained C₁₋₆ alkyl heteroalkylene having one or two oxygen atoms, such as 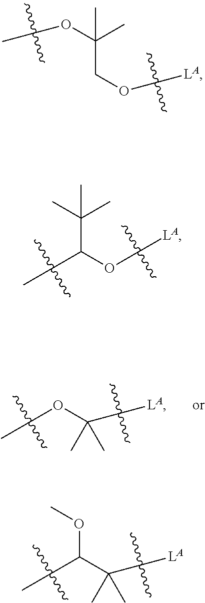
(L^{A} is shown to show direction of connection).

45. The compound of any of claims 42-44, or a pharmaceutically acceptable salt or ester thereof, wherein p1 is 0, or p1 is 1.

46. The compound of any of claims 42-45, or a pharmaceutically acceptable salt or ester thereof, wherein R^{A} at each occurrence is independently F, Cl, CN, C₁₋₄ alkyl optionally substituted with 1-3 fluorine, or C₁₋₄ alkoxy optionally substituted with 1-3 fluorine.

47. The compound of any of claims 42-46, or a pharmaceutically acceptable salt or ester thereof, wherein p2 is 0.

48. The compound of any of claims 42-46, or a pharmaceutically acceptable salt or ester thereof, wherein p2 is 1, and R^{C} is F, Cl, CN, C₁₋₄ alkyl optionally substituted with 1-3 fluorine, or C₁₋₄ alkoxy optionally substituted with 1-3 fluorine.

49. The compound of any of claims 42-48, or a pharmaceutically acceptable salt or ester thereof, wherein Y is CH.

50. The compound of any of claims 42-49, or a pharmaceutically acceptable salt or ester thereof, wherein Z is O.

51. The compound of any of claims 42-50, or a pharmaceutically acceptable salt or ester thereof, wherein R¹¹ and R¹² are both hydrogen.

52. The compound of any of claims 42-51, or a pharmaceutically acceptable salt or ester thereof, wherein L¹⁰ is 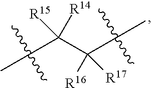
wherein CR₁₆R¹⁷ is bonded to the COOH group, and wherein:
R¹⁴ is hydrogen, C₁₋₆ alkyl, C₂₋₆ alkenyl, C₂₋₆ alkynyl, C₃₋₆ cycloalkyl, phenyl, 5 or 6 membered heteroaryl, or 3-7 membered heterocyclyl, wherein each of the alkyl, alkenyl, alkynyl, cycloalkyl, phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy, and R¹⁵, R¹⁶ and R¹⁷ are each independently hydrogen or C₁₋₄ alkyl; or
R¹⁴ and R¹⁵ are joined to form a 3-7 membered ring with 0, 1, or 2 heteroatoms selected from O, N, or S.

53. The compound of any of claims 42-52, or a pharmaceutically acceptable salt or ester thereof, wherein R¹³ is a phenyl ring, which is unsubstituted or substituted with 1-3 substituents independently selected from F, Cl, CN, OH, C₁₋₆ alkyl, C₁₋₆ alkyl heteroalkyl, C₃₋₆ cycloalkyl, C₁₋₆ alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), and C₁₋₄ alkyl optionally substituted with F.

54. The compound of any of claims 42-52, or a pharmaceutically acceptable salt or ester thereof, wherein R¹³ is a 6-membered heteroaryl ring, such as a pyridyl ring, which is unsubstituted or substituted with 1-3 substituents independently selected from F, Cl, CN, OH, C₁₋₆ alkyl, C₁₋₆ heteroalkyl, C₃₋₆ cycloalkyl, C₁₋₆ alkyl alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), and C₁₋₄ alkyl optionally substituted with F.

55. The compound of any of claims 42-44, or a pharmaceutically acceptable salt or ester thereof, which has a Formula II-1-A or Formula II-B-2: 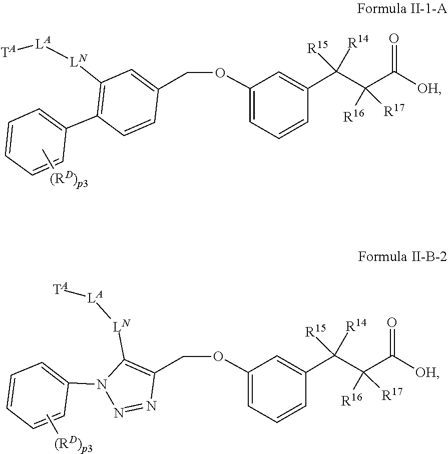
wherein:
R¹⁴ is hydrogen, C₁₋₆ alkyl, C₂₋₆ alkenyl, C₂₋₆ alkynyl, C₃₋₆ cycloalkyl, phenyl, 5 or 6 membered heteroaryl, or 3-7 membered heterocyclyl, wherein each of the alkyl, alkenyl, alkynyl, cycloalkyl, phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy, and R¹⁵, R¹⁶ and R¹⁷ are each independently hydrogen or C₁₋₄ alkyl; or
R¹⁴ and R¹⁵ are joined to form a 3-7 membered ring with 0, 1, or 2 heteroatoms selected from O, N, or S;
R^{D} at each occurrence is independently F, Cl, C₁₋₄ alkyl optionally substituted with 1-3 F, or C₁₋₄ alkoxy optionally substituted with 1-3 F, and
wherein p3 is 0, 1, 2, or 3.

56. The compound of claim 55, or a pharmaceutically acceptable salt or ester thereof, wherein R¹⁶ and R¹⁷ are both hydrogen, or one of R¹⁶ and R¹⁷ is hydrogen and the other of R¹⁶ and R¹⁷ is methyl.

57. The compound of claim 55 or 56, or a pharmaceutically acceptable salt or ester thereof, wherein one of R¹⁴ and R¹⁵ is hydrogen, and the other of R¹⁴ and R¹⁵ is C₁₋₆ alkyl, C₂₋₆ alkenyl, C₂₋₆ alkynyl, or C₃₋₆ cycloalkyl.

58. The compound of claim 55 or 56, or a pharmaceutically acceptable salt or ester thereof, wherein R¹⁴ and R¹⁵ are joined to form a C₃₋₆ cycloalkyl.

59. The compound of any of claims 42-44, or a pharmaceutically acceptable salt or ester thereof, wherein q is 1 and the compound has a Formula II-1-A-1 or II-B-3:

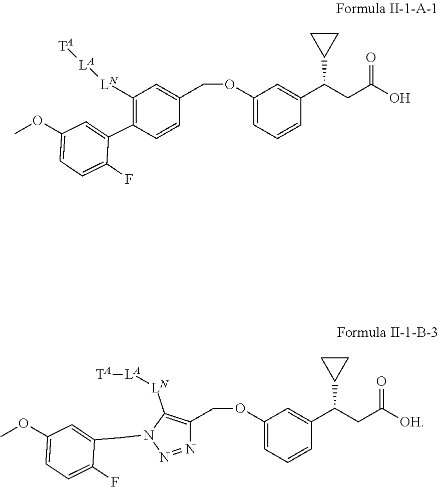

60. The compound of any of claims 42-59, or a pharmaceutically acceptable salt or ester thereof, wherein L^{A} is characterized in that the maximum length between the two end atoms of L^{A} is at least the maximum length between the two end carbon atoms of —(CH₂)₁₂—, preferably, at least that of —(CH₂)₁₄—, more preferably, at least that of —(CH₂)₁₆—.

61. The compound of any of claims 42-59, or a pharmaceutically acceptable salt or ester thereof, wherein L^{A} is characterized in that the maximum length between the two end atoms of L^{A} is between (i) the maximum length between the two end carbon atoms of —(CH₂)₁₂— and (ii) the maximum length between the two end carbon atoms of —(CH₂)₅₀—.

62. The compound of any of claims 42-61, or a pharmaceutically acceptable salt or ester thereof, wherein only one end atom of L^{A} is C of a C(O) or S of a SO₂ group.

63. The compound of claim 62, or a pharmaceutically acceptable salt or ester thereof, wherein L^{A} is characterized in that the corresponding compound H-L^{A}-OH has a cLogP of at least 4 (e.g., at least 4.5, at least 5, at least 5.5, at least 6, at least 6.5, at least 7, etc.), wherein the —OH is bonded with the end C(O) or SO₂ group.

64. The compound of claim 62, or a pharmaceutically acceptable salt or ester thereof, wherein L^{A} is characterized in that the corresponding compound H-L^{A}-OH has a cLogP of between 3-15 (e.g., 3-10, 4-12, 4.5-9, 5-11, 5.5-10.5, 6-14, 6.5-13, etc.), wherein the —OH is bonded with the end C(O) or SO₂ group.

65. The compound of any of claims 42-64, or a pharmaceutically acceptable salt or ester thereof, wherein (1) the terminal atom(s) is N of a basic amine group, and T^{A} is characterized in that the corresponding compound T^{A}-(C(O)—CH₃)_{q} has a cLogP of less than 0 (e.g., less than −1, less than −2, less than −3, less than −3.5, less than −4, or even lower); (2) the terminal atom(s) is C of a C(O) group, and T^{A} is characterized in that the corresponding compound T^{A}-(OH) q has a cLogP of less than 0 (e.g., less than −1, less than −2, less than −3, less than −3.5, less than −4, or even lower); (3) the terminal atom(s) is S in a SO₂ group, and T^{A} is characterized in that the corresponding compound T^{A}-(OH)_{q} has a cLogP of less than 0 (e.g., less than −1, less than −2, less than −3, less than −3.5, less than −4, or even lower); or (4) the terminal atom(s) is not N of a basic amine group, C of a C(O) group, or S in a SO₂ group, and T^{A} is characterized in that the corresponding compound T^{A}-H_{q} has a cLogP of less than 0 (e.g., less than −1, less than −2, less than −3, less than −3.5, less than −4, or even lower).

66. The compound of any of claims 42-64, or a pharmaceutically acceptable salt or ester thereof, wherein T^{A1}, T^{A1}-L^{B}-, T^{A1}-L^{B}-(heteroalkylene), or T^{A1}-L^{B}-(heteroalkylene)-L^{B}-,
wherein L^{B} at each occurrence is independently —SO₂—, —C(═O)—, or a moiety selected from: 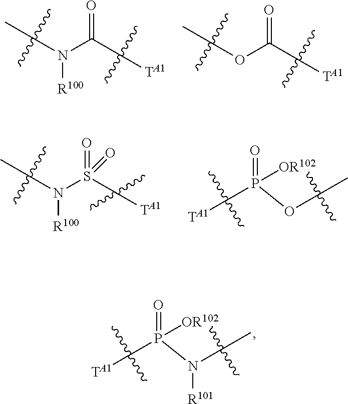
wherein R¹⁰⁰, R¹⁰¹ and R¹⁰² at each occurrence is independently hydrogen, optionally substituted alkyl, or optionally substituted cycloalkyl,
wherein T^{A1} is characterized as having a structure of: 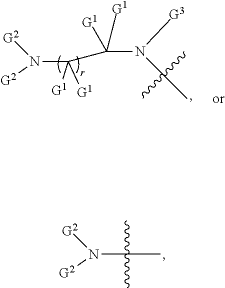
wherein G¹ at each occurrence is independently hydrogen, halogen, optionally substituted C₁₋₄ alkyl, or optionally substituted C₁₋₄ heteroalkyl (such as optionally substituted C₁₋₄ alkoxy), or two gem G¹ group can join to form an ═O, ═NH, or ═N(C₁₋₄ alkyl), or two or more G¹ groups can join to form a ring structure,
the integer r is 1-10, such as 1 or 2,
G² at each occurrence is independently hydrogen, G¹⁰, C(O)-G¹⁰, SO₂G¹⁰, C(O)—NH-G¹⁰, or SO₂NHG¹⁰, C(O)—O-G¹⁰, or SO₂OG¹⁰, wherein G¹⁰ at each occurrence is independently an optionally substituted alkyl, an optionally substituted heteroalkyl, or an optionally substituted 3-10 membered ring structure, which can be carbocyclic, heterocyclic, aromatic, heteroaromatic, or a combination thereof,
G³ is hydrogen or an optionally substituted alkyl (e.g., C₁₋₄ alkyl);
or two G² or one G² and one G¹ or one G¹ and G³ can be joined to form a ring structure such as a 3-10 membered ring structure,
wherein the fragment 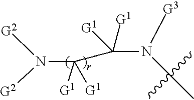
is further characterized in that (i) when the CG¹G¹ group next to the NG³ is not C(═O), the corresponding compound 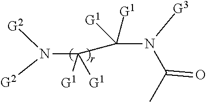
has a cLogP less than 1, preferably, less than 0 (e.g., less than −1, less than −2, less than −3, less than −3.5, less than −4, or even lower), or (ii) when the CG¹G¹ group next to the NG³ is C(═O), the corresponding compound 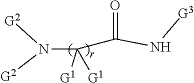
has a cLogP less than 1, preferably, less than 0 (e.g., less than −1, less than −2, less than −3, less than −3.5, less than −4, or even lower), and
wherein the fragment 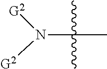
is further characterized in that (i) when the nitrogen is a basic nitrogen, the corresponding compound (G²)₂N—C(O)—CH₃ has a cLogP less than 1, preferably, less than 0 (e.g., less than −1, less than −2, less than −3, less than −3.5, less than −4, or even lower), or (ii) when the nitrogen is a nonbasic nitrogen, the corresponding compound (G²)₂N—H has a cLogP less than 1, preferably, less than 0 (e.g., less than −1, less than −2, less than −3, less than −3.5, less than −4, or even lower).

67. The compound of any of claims 42-64, or a pharmaceutically acceptable salt or ester thereof, wherein T^{A} is T^{A1}, T^{A1}-L^{B}-, T^{A1}-L^{B}-(heteroalkylene), or T^{A1}-L^{B}-(heteroalkylene)-L^{B}-,
wherein L^{B} at each occurrence is independently —SO₂—, —C(═O)—, or a moiety selected from: 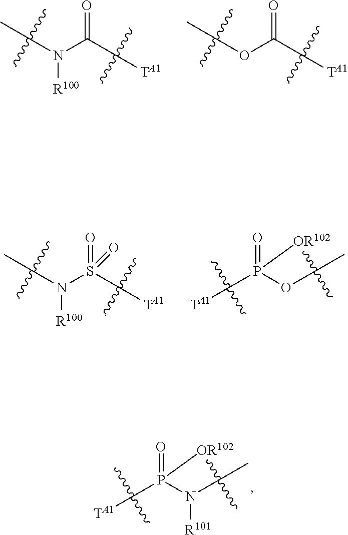
wherein R¹⁰⁰, R¹⁰¹ and R¹⁰² at each occurrence is independently hydrogen, optionally substituted alkyl, or optionally substituted cycloalkyl,
wherein T^{A1} is characterized as having a structure of: 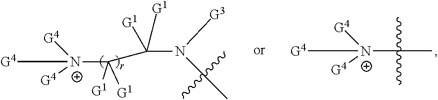
charge balanced with a counterion (e.g., described herein) as necessary,
wherein G¹ at each occurrence is independently hydrogen, halogen, optionally substituted C₁₋₄ alkyl, or optionally substituted C₁₋₄ heteroalkyl (such as optionally substituted C₁₋₄ alkoxy), or two gem G¹ group can join to form an ═O, ═NH, or ═N(C₁₋₄ alkyl), or two or more G¹ groups can join to form a ring structure,
the integer r is 1-10, such as 1 or 2,
G³ is hydrogen or an optionally substituted alkyl (e.g., C₁₋₄ alkyl);
G⁴ at each occurrence is independently hydrogen or G¹⁰, wherein G¹⁰ at each occurrence is independently an optionally substituted alkyl, an optionally substituted heteroalkyl, or an optionally substituted 3-10 membered ring structure, which can be carbocyclic, heterocyclic, aromatic, heteroaromatic, or a combination thereof, or two G⁴ or one G⁴ and one G¹ or one G¹ and G³ can be joined to form an optionally substituted 3-10 membered ring structure,
wherein the fragment 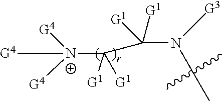
is characterized in that (i) when the CG¹G¹ group next to the NH is not C(═O), the corresponding compound 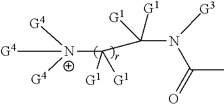
has a cLogP less than 1, preferably, less than 0 (e.g., less than −1, less than −2, less than −3, less than −3.5, less than −4, or even lower), or (ii) when the CG¹G¹ group next to the NH is C(═O), the corresponding compound 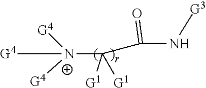
has a cLogP less than 1, preferably, less than 0 (e.g., less than −1, less than −2, less than −3, less than −3.5, less than −4, or even lower).

68. The compound of claim 66 or 67, or a pharmaceutically acceptable salt or ester thereof, wherein G³ is hydrogen and/or G¹ at each occurrence is hydrogen.

69. The compound of any of claims 42-68, or a pharmaceutically acceptable salt or ester thereof, wherein T^{A} is characterized as having a charged group (including zwitterion structures) or a group that can become charged at pH 7, such as primary amine, secondary amine, tertiary amine, quaternary amine, carboxylic acid, etc.

70. The compound of any of claims 42-69, or a pharmaceutically acceptable salt or ester thereof, wherein T^{A} is characterized as having at least two hydrogen bond donors.

71. The compound of any of claims 42-70, or a pharmaceutically acceptable salt or ester thereof, wherein T^{A} is characterized as having at least two hydrogen bond acceptors.

72. The compound of any of claims 42-71, or a pharmaceutically acceptable salt or ester thereof, wherein the covalent bond(s) between the terminal atom(s) of T^{A} and the first end atom of L^{A} is an amide bond.

73. The compound of any of claims 42-71, or a pharmaceutically acceptable salt or ester thereof, wherein the covalent bond(s) between the terminal atom(s) of T^{A} and the first end atom of L^{A} is a non-amide carbon-nitrogen bond, an ester bond, a non-ester carbon-oxygen bond, a carbon-carbon bond, or a carbon-sulfur bond.

74. The compound of any of claims 42-73, or a pharmaceutically acceptable salt or ester thereof, wherein L^{A} is (X) m, wherein X at each occurrence is independently CR₂, C(═O), —C(R)═C(R)—, 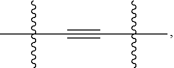 [SMILES: *C#C*]
SiR₂, O, S, SO₂, NR, [NR₂]⁺, or a ring structure, preferably 3-10 membered ring structure, wherein R at each occurrence is independently hydrogen, halogen, optionally substituted C₁₋₄ alkyl, or optionally substituted C₁₋₄ alkoxy, typically R is hydrogen or C₁₋₄ alkyl, and the integer m is at least 10, such as at least 12, at least 14, at least 16, at least 18, at least 20, at least 50, such as 12-50, 16-50, etc.

75. The compound of any of claims 42-74, or a pharmaceutically acceptable salt or ester thereof, wherein L^{A} is —(X)ₘ₋₁—C(O)—, wherein the C(O) end is bonded with T^{A}, wherein X at each occurrence is independently CR₂, C(═O), —C(R)═C(R)—, 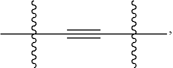 [SMILES: *C#C*]
SiR₂, O, S, SO₂, NR, [NR₂]⁺ or a ring structure, preferably 3-10 membered ring structure, provided that the end X group is not C(O) or SO₂, wherein R at each occurrence is independently hydrogen, halogen, optionally substituted C₁₋₄ alkyl, or optionally substituted C₁₋₄ alkoxy, typically R is hydrogen or C₁₋₄ alkyl, and the integer m is at least 10, such as at least 12, at least 14, at least 16, at least 18, at least 20, at least 50, such as 12-50, 16-50, etc., and the hydrophobicity of —(X)ₘ₋₁—C(O)— is characterized in that the corresponding compound H—(X)ₘ₋₁—COOH has a cLogP of at least 3, such as between 3-15, preferably, at least 4.

76. The compound of any of claims 42-75, or a pharmaceutically acceptable salt or ester thereof, wherein L^{A} is —C₁₂₋₃₀ alkylene- or —C₁₂₋₃₀ alkylene-C(O)—, wherein the —C₁₂₋₃₀ alkylene- is optionally substituted, wherein the optional substituents can optionally be joined together to form a double bond, triple bond, or a ring structure.

77. The compound of claim 76, or a pharmaceutically acceptable salt or ester thereof, wherein L^{A} or L^{N}-L^{A} has a structure of 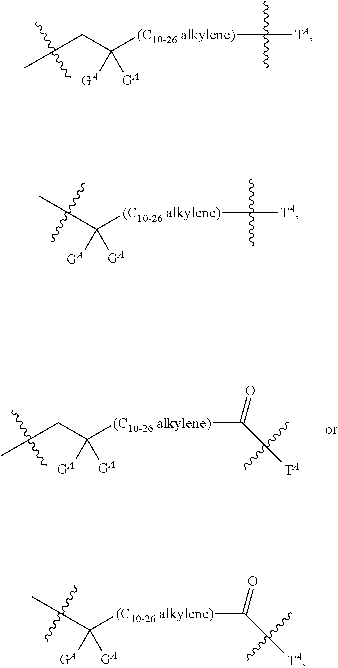
wherein the C₁₀₋₂₆ alkylene is optionally substituted, and wherein G^{A} at each occurrence is independently hydrogen or an optionally substituted C₁₋₄ alkyl, or two G^{A} are joined to form a 3-6 membered ring, such as a cyclopropyl or cyclobutyl ring, preferably, G^{A} at each occurrence is methyl.

78. The compound of any of claims 42-75, or a pharmaceutically acceptable salt or ester thereof, wherein L^{A} is a 12-30 membered heteroalkylene or -(12-30 membered heteroalkylene)-C(O)—, wherein the 12-30 membered heteroalkylene is optionally substituted and contains 1-6 heteroatoms independently selected from O, N, and S, wherein the sulfur atom(s), if present, is optionally oxidized, wherein the optional substituents can optionally be joined together to form a double bond, triple bond, or a ring structure.

79. The compound of any of claims 42-75, or a pharmaceutically acceptable salt or ester thereof, wherein L^{A} or L^{N}-L^{A} has a structure of 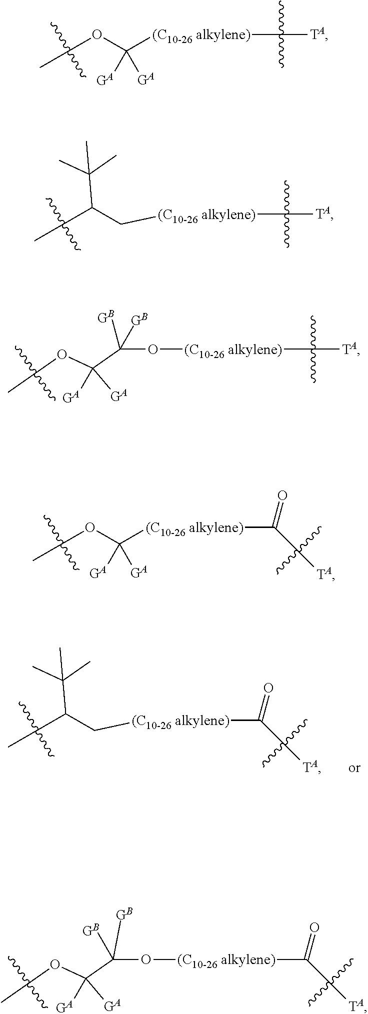
wherein the C₁₀₋₂₆ alkylene is optionally substituted, and wherein G^{A} at each occurrence is independently hydrogen or an optionally substituted C₁₋₄ alkyl, or two G^{A} are joined to form a 3-6 membered ring, such as a cyclopropyl or cyclobutyl ring, wherein G^{B} at each occurrence is independently hydrogen or an optionally substituted C₁₋₄ alkyl, or two G^{B} or one G^{A} and one G^{B} are joined to form a 3-6 membered ring, such as a cyclopropyl or cyclobutyl ring.

80. The compound of any of claims 42-75, or a pharmaceutically acceptable salt or ester thereof, wherein L^{A} is 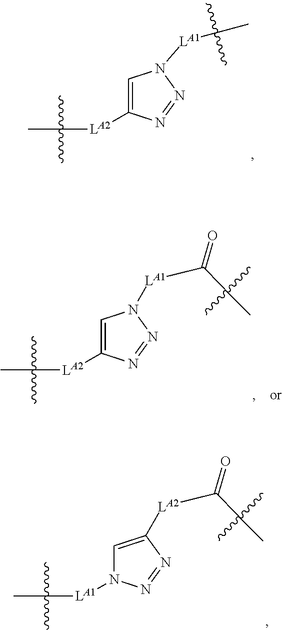
wherein L^{A1} and L^{A2} are each independently a bond, an optionally substituted —C₁₋₃₀ alkylene-, or an optionally substituted —C₁₋₃₀ heteroalkylene-containing 1-6 heteroatoms independently selected from O, N, and S, wherein the sulfur atom(s), if present, is optionally oxidized, wherein the optional substituents can optionally be joined together to form a double bond, triple bond, or a ring structure, provided that the longest chain length of L^{A} is at least that of (CH₂)₁₂—, such as at least that of —(CH₂)₁₄—, at least that of —(CH₂)₁₆—, at least that of —(CH₂)₁₈—.

81. The compound of any of claims 42-75, or a pharmaceutically acceptable salt or ester thereof, wherein L^{A} or L^{N}-L^{A} has a structure of 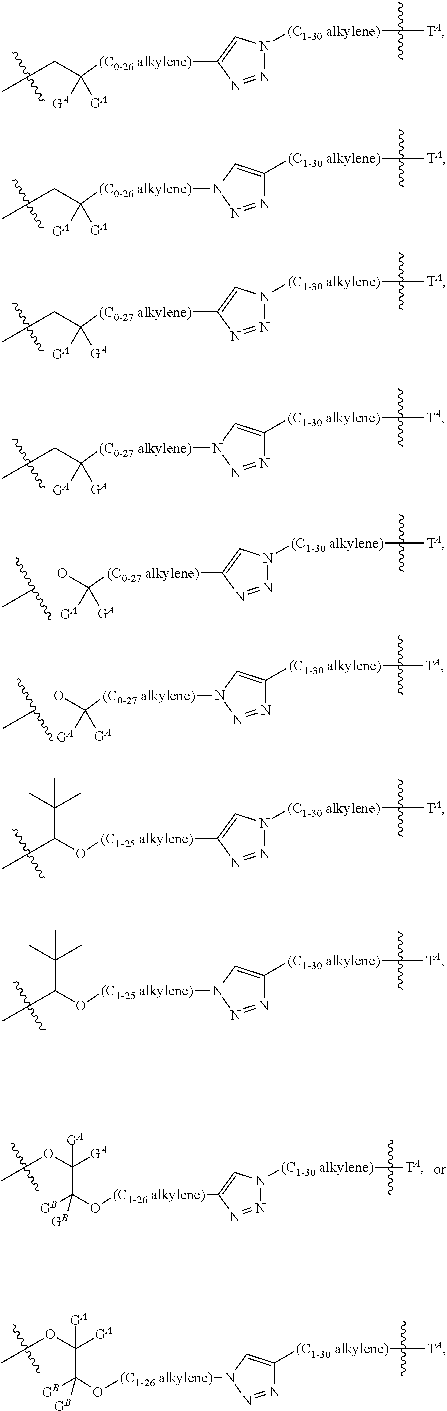
wherein the C₀₋₂₆ alkylene, C₀₋₂₇ alkylene, C₁₋₂₅ alkylene, C₁₋₂₆ alkylene, or C₁₋₃₀ alkylene is optionally substituted, and wherein G^{A} at each occurrence is independently hydrogen or an optionally substituted C₁₋₄ alkyl, or two G^{A} are joined to form a 3-6 membered ring, such as a cyclopropyl or cyclobutyl ring, wherein G^{B} at each occurrence is independently hydrogen or an optionally substituted C₁₋₄ alkyl, or two G^{B} or one G^{A} and one G^{B} are joined to form a 3-6 membered ring, such as a cyclopropyl or cyclobutyl ring, preferably, G^{A} at each occurrence is methyl, preferably, G^{B} at each occurrence is hydrogen.

82. The compound of any of claims 42-75, or a pharmaceutically acceptable salt or ester thereof, wherein L^{A} or L^{N}-L^{A} has a structure of 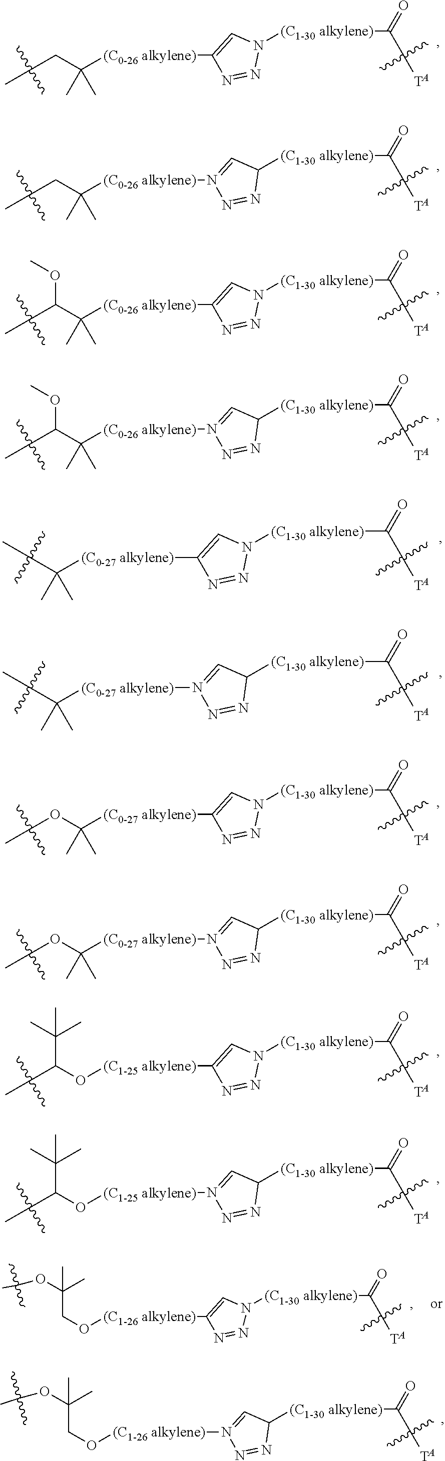
wherein the C₀₋₂₆ alkylene, C₀₋₂₇ alkylene, C₁₋₂₅ alkylene, C₁₋₂₆ alkylene, or C₁₋₃₀ alkylene is optionally substituted.

83. The compound of any of claims 42-82, or a pharmaceutically acceptable salt or ester thereof, wherein T^{A} is T^{A1} or T^{A1}—C(O)—, wherein T^{A1} is 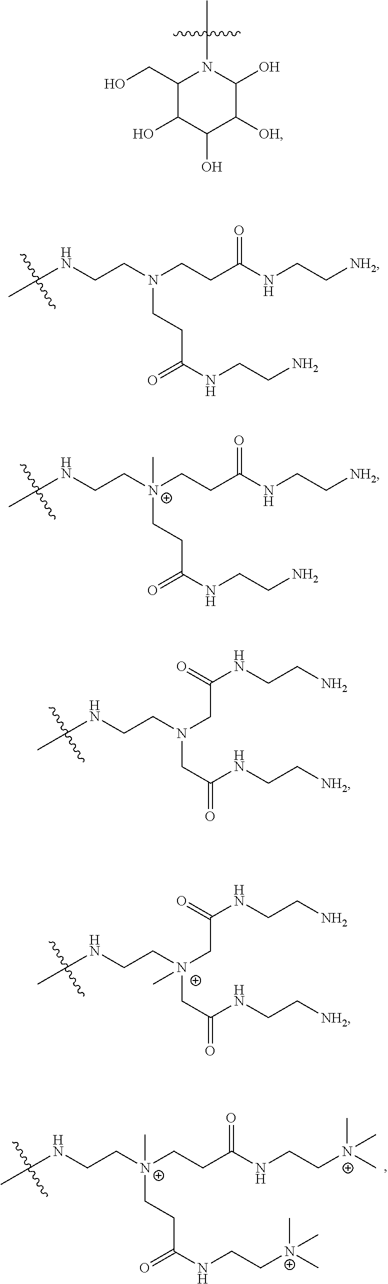 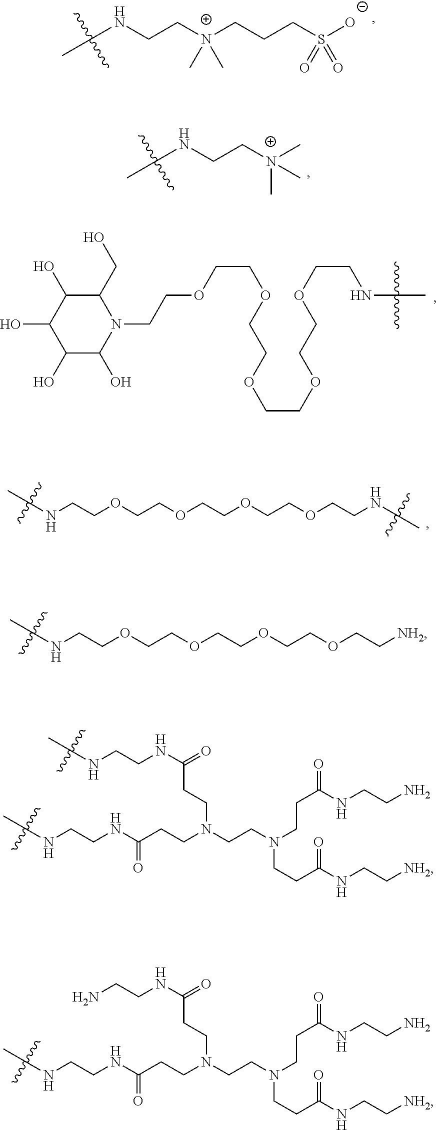 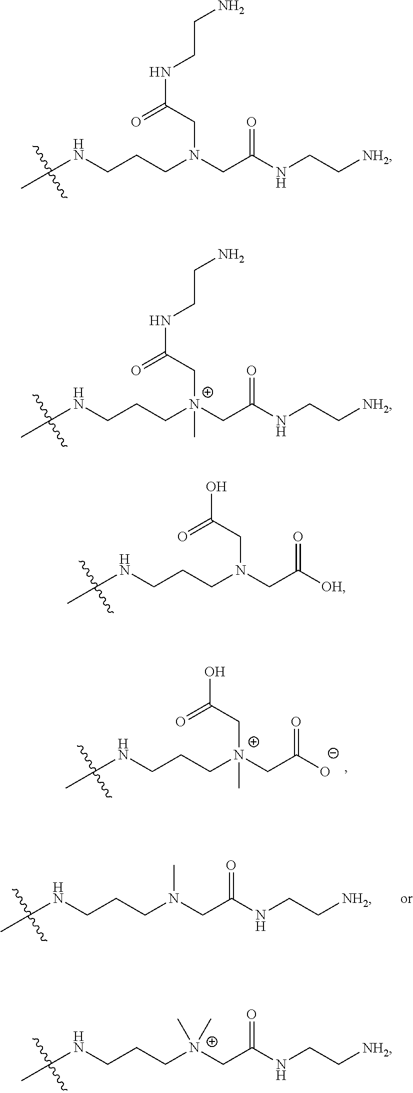
wherein T^{A} is charge balanced with a counterion (e.g., described herein) as necessary.

84. A compound of Formula III or III-B, or a pharmaceutically acceptable salt or ester thereof: 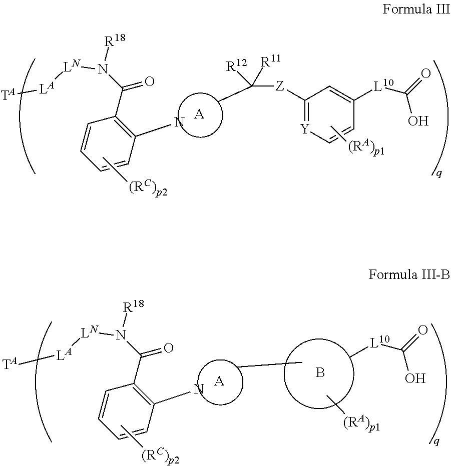
wherein:
Y is CH, CR^{A}, or N;
Z is O, S, NH, or N(C₁₋₄ alkyl);
R¹¹ and R¹² are each independently hydrogen or C₁₋₄ alkyl;
Ring A is an optionally substituted 4-12 membered nitrogen-containing ring;
Ring B is an optionally substituted monocyclic heteroaryl or a bicyclic aryl or heteroaryl ring, such as a benzofuran ring;
L^{N} is null, an optionally substituted C₁₋₆ alkylene, or an optionally substituted C₁₋₆ heteroalkylene having 1-3 heteroatoms;
L¹⁰ is an alkylene, optionally substituted with 1-3 substituents independently selected from halogen, optionally substituted C₁₋₆ alkyl, optionally substituted C₂₋₆ alkenyl, optionally substituted C₂₋₆ alkynyl, optionally substituted C₁₋₆ heteroalkyl, optionally substituted C₃₋₆ cycloalkyl, optionally substituted C₁₋₆ alkoxy, optionally substituted C₃₋₆ cycloalkoxy, optionally substituted heterocyclyl, optionally substituted aryl, and optionally substituted heteroaryl, or two substituents are joined to form an optionally substituted ring structure;
R^{A} at each occurrence is independently halogen, CN, optionally substituted C₁₋₆ alkyl, optionally substituted C₃₋₆ cycloalkyl, optionally substituted C₁₋₆ alkoxy, or optionally substituted C₃₋₆ cycloalkoxy, or two R^{A} are joined to form an optionally substituted ring structure; p1 is 0, 1, or 2;
R^{C} at each occurrence is independently halogen, CN, optionally substituted C₁₋₆ alkyl, optionally substituted C₃₋₆ cycloalkyl, optionally substituted C₁₋₆ alkoxy, or optionally substituted C₃₋₆ cycloalkoxy, or two R^{C} are joined to form an optionally substituted ring structure; p2 is 0, 1, 2, or 3;
R¹⁸ is an optionally substituted phenyl or an optionally substituted heteroaryl, and
wherein:
q is an integer of 1-10, preferably, 1 or 2,
T^{A} is a hydrophilic group having a terminal atom(s) selected from N, O, S, or C, which is covalently bonded with a first end atom of L^{A}, wherein (1) when the terminal atom(s) is N of a basic amine group, then the corresponding compound T^{A}-(C(O)—CH₃)_{q} has a cLogP of less than 1, wherein the —C(O)—CH₃ is bonded with the terminal N atom(s); (2) when the terminal atom(s) is C of a C(O) group, then the corresponding compound T^{A}-(OH)_{q} has a cLogP of less than 1, wherein the —OH is bonded with the terminal C atom(s); (3) when the terminal atom(s) is S of a SO₂ group, then the corresponding compound T^{A}-(OH)_{q} has a cLogP of less than 1, wherein the —OH is bonded with the terminal S atom(s); or (4) when (1)-(3) do not apply, then the corresponding compound T^{A}-H_{q} has a cLogP of less than 1; and
L^{A} is a linker characterized in that the maximum length between the two end non-hydrogen atoms of L^{A} is at least the maximum length between the two end carbon atoms of —(CH₂)₁₀—, wherein (1) only one end atom of L^{A} is C of a C(O) or S of a SO₂ group, which is bonded with T^{A}, and the corresponding compound H-L^{A}-OH has a cLogP of at least 3, wherein the —OH is bonded with the end C(O) or SO₂ group; or (2) neither end atoms of L^{A} is C of a C(O) or S of a SO₂ group, and the corresponding compound H-L^{A}-H has a cLogP of at least 3.

85. The compound of claim 84, or a pharmaceutically acceptable salt or ester thereof, wherein q is 1 and the compound has a Formula III-1 or III-B-1:

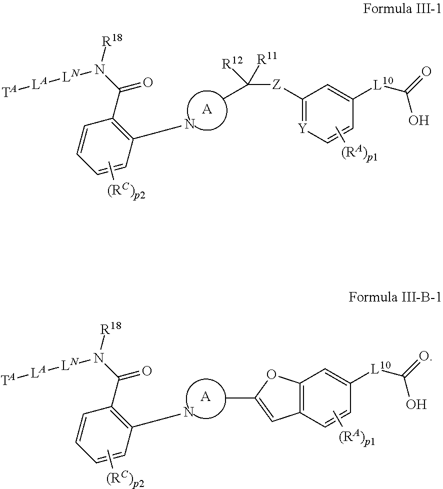

86. The compound of claim 84 or 85, or a pharmaceutically acceptable salt or ester thereof, wherein L^{N} is (i) null; or (ii) a branched or straight chained C₁₋₆ alkyl alkylene, such as 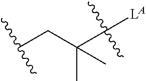
(L^{A} is shown to show direction of connection).

87. The compound of any of claims 84-86, or a pharmaceutically acceptable salt or ester thereof, wherein p1 is 0 or p1 is 1.

88. The compound of any of claims 84-87, or a pharmaceutically acceptable salt or ester thereof, wherein R^{A} at each occurrence is independently F, Cl, CN, C₁₋₄ alkyl optionally substituted with 1-3 fluorine, or C₁₋₄ alkoxy optionally substituted with 1-3 fluorine.

89. The compound of any of claims 84-88, or a pharmaceutically acceptable salt or ester thereof, wherein p2 is 0.

90. The compound of any of claims 84-88, or a pharmaceutically acceptable salt or ester thereof, wherein p2 is 1, and R^{C} is F, Cl, CN, C₁₋₄ alkyl optionally substituted with 1-3 fluorine, or C₁₋₄ alkoxy optionally substituted with 1-3 fluorine.

91. The compound of any of claims 84-90, or a pharmaceutically acceptable salt or ester thereof, wherein Y is N.

92. The compound of any of claims 84-91, or a pharmaceutically acceptable salt or ester thereof, wherein Z is O.

93. The compound of any of claims 84-92, or a pharmaceutically acceptable salt or ester thereof, wherein R¹¹ and R¹² are both hydrogen.

94. The compound of any of claims 84-93, or a pharmaceutically acceptable salt or ester thereof, wherein L¹⁰ is 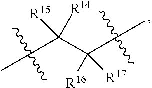
wherein CR₁₆R¹⁷ is bonded to the COOH group, and wherein:
R¹⁴ is hydrogen, C₁₋₆ alkyl, C₂₋₆ alkenyl, C₂₋₆ alkynyl, C₃₋₆ cycloalkyl, phenyl, 5 or 6 membered heteroaryl, or 3-7 membered heterocyclyl, wherein each of the alkyl, alkenyl, alkynyl, cycloalkyl, phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy, and R¹⁵, R¹⁶ and R¹⁷ are each independently hydrogen or C₁₋₄ alkyl; or
R¹⁴ and R¹⁵ are joined to form a 3-7 membered ring with 0, 1, or 2 heteroatoms selected from O, N, or S.

95. The compound of any of claims 84-94, or a pharmaceutically acceptable salt or ester thereof, wherein R¹⁸ is a 6-membered heteroaryl ring, e.g., 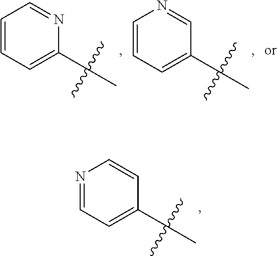 [SMILES: *c1ccccn1 ; *c1cccnc1 ; *c1ccncc1]
which is optionally substituted with 1-3 substituents independently selected from F, Cl, CN, OH, C₁₋₆ alkyl, C₁₋₆ alkyl heteroalkyl, C₃₋₆ cycloalkyl, C₁₋₆ alkyl alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), and C₁₋₄ alkyl optionally substituted with F.

96. The compound of any of claims 84-95, or a pharmaceutically acceptable salt or ester thereof, wherein Ring A is a 4-8 membered optionally substituted monocyclic saturated heterocyclic ring having one or two ring heteroatoms independently selected from S, O, and N, provided at least one of the ring heteroatom is nitrogen.

97. The compound of claim 96, or a pharmaceutically acceptable salt or ester thereof, wherein Ring A is selected from: 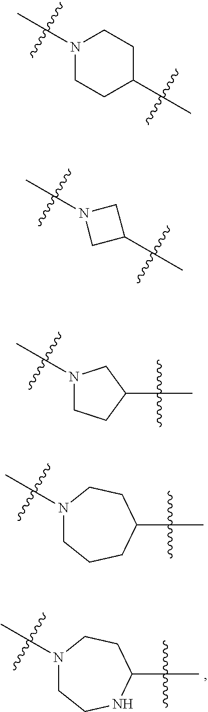 [SMILES: *C1CCN(*)CC1 ; *C1CN(*)C1 ; *C1CCN(*)C1 ; *C1CCCN(*)CC1 ; *C1CCN(*)CCN1]
each of which is optionally substituted with 1-2 substituents independently selected from F, OH, NH₂, NH(C₁₋₄ alkyl), N(C₁₋₄ alkyl)(C₁₋₄ alkyl), C₁₋₄ alkyl optionally substituted with 1-3 fluorine, or C₁₋₄ alkoxy optionally substituted with 1-3 fluorine.

98. The compound of any of claims 84-95, or a pharmaceutically acceptable salt or ester thereof, wherein Ring A is bicyclic or polycyclic 6-12 membered optionally substituted saturated heterocyclic ring having one or two ring heteroatoms independently selected from S, O, and N, provided at least one of the ring heteroatom is nitrogen.

99. The compound of any of claims 84-86, or a pharmaceutically acceptable salt or ester thereof, which has a Formula III-1-A or III-B-2: 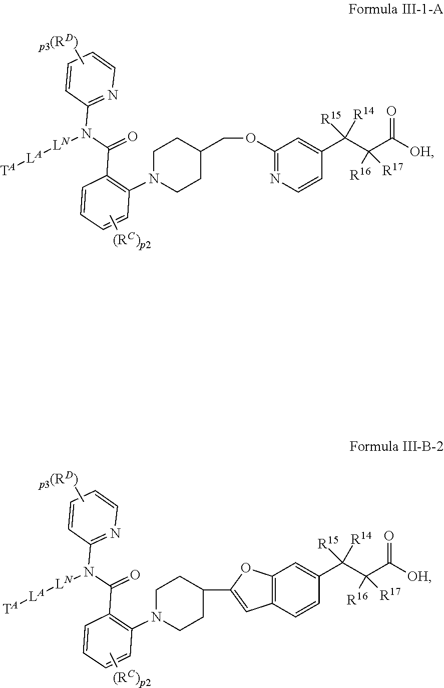
wherein:
R¹⁴ is hydrogen, C₁₋₆ alkyl, C₂₋₆ alkenyl, C₂₋₆ alkynyl, C₃₋₆ cycloalkyl, phenyl, 5 or 6 membered heteroaryl, or 3-7 membered heterocyclyl, wherein each of the alkyl, alkenyl, alkynyl, cycloalkyl, phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy, and R¹⁵, R¹⁶ and R¹⁷ are each independently hydrogen or C₁₋₄ alkyl; or
R¹⁴ and R¹⁵ are joined to form a 3-7 membered ring with 0, 1, or 2 heteroatoms selected from O, N, or S;
R^{D} at each occurrence is independently F, Cl, C₁₋₄ alkyl optionally substituted with 1-3 F, or C₁₋₄ alkoxy optionally substituted with 1-3 F, and
wherein p3 is 0, 1, 2, or 3.

100. The compound of claim 99, or a pharmaceutically acceptable salt or ester thereof, wherein R¹⁶ and R¹⁷ are both hydrogen, or one of R¹⁶ and R¹⁷ is hydrogen and the other of R¹⁶ and R¹⁷ is methyl.

101. The compound of claim 99 or 100, or a pharmaceutically acceptable salt or ester thereof, wherein one of R¹⁴ and R¹⁵ is hydrogen, and the other of R¹⁴ and R¹⁵ is C₁₋₆ alkyl, C₂₋₆ alkenyl, C₂₋₆ alkynyl, or C₃₋₆ cycloalkyl.

102. The compound of claim 99 or 100, or a pharmaceutically acceptable salt or ester thereof, wherein R¹⁴ and R¹⁵ are joined to form a C₃₋₆ cycloalkyl.

103. The compound of any of claims 84-86, or a pharmaceutically acceptable salt or ester thereof, wherein q is 1 and the compound has a Formula III-1-A-1 or III-B-3:

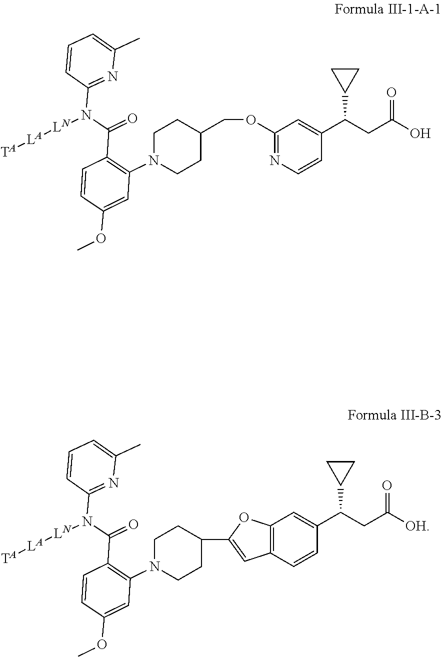

104. The compound of any of claims 84-103, or a pharmaceutically acceptable salt or ester thereof, wherein L^{A} is characterized in that the maximum length between the two end atoms of L^{A} is at least that between the two end carbon atoms of —(CH₂)₁₂—, preferably, at least that of —(CH₂)₁₄—, more preferably, at least that of —(CH₂)₁₆—.

105. The compound of any of claims 84-103, or a pharmaceutically acceptable salt or ester thereof, wherein L^{A} is characterized in that the maximum length between the two end atoms of L^{A} is between (i) the maximum length between the two end carbon atoms of —(CH₂)₁₂— and (ii) the maximum length between the two end carbon atoms of —(CH₂)₅₀—.

106. The compound of any of claims 84-105, or a pharmaceutically acceptable salt or ester thereof, wherein only one end atom of L^{A} is C of a C(O) or S of a SO₂ group, which is bonded with T^{A}.

107. The compound of claim 106, or a pharmaceutically acceptable salt or ester thereof, wherein L^{A} is characterized in that the corresponding compound H-L^{A}-OH has a cLogP of at least 4, wherein the —OH is bonded with the end C(O) or SO₂ group.

108. The compound of claim 106, or a pharmaceutically acceptable salt or ester thereof, wherein L^{A} is characterized in that the corresponding compound H-L^{A}-OH has a cLogP of between 3-15, wherein the —OH is bonded with the end C(O) or SO₂ group.

109. The compound of any of claims 84-108, or a pharmaceutically acceptable salt or ester thereof, wherein (1) the terminal atom(s) is N of a basic amine group, and T^{A} is characterized in that the corresponding compound T^{A}-(C(O)—CH₃)_{q} has a cLogP of less than 0 (e.g., less than −1, less than −2, less than −3, less than −3.5, less than −4, or even lower); (2) the terminal atom(s) is C of a C(O) group, and T^{A} is characterized in that the corresponding compound T^{A}-(OH)_{q} has a cLogP of less than 0 (e.g., less than −1, less than −2, less than −3, less than −3.5, less than −4, or even lower); (3) the terminal atom(s) is S in a SO₂ group, and T^{A} is characterized in that the corresponding compound T^{A}-(OH)_{q} has a cLogP of less than 0 (e.g., less than −1, less than −2, less than −3, less than −3.5, less than −4, or even lower); or (4) the terminal atom(s) is not N of a basic amine group, C of a C(O) group, or S in a SO₂ group, and T^{A} is characterized in that the corresponding compound T^{A}-H_{q} has a cLogP of less than 0 (e.g., less than −1, less than −2, less than −3, less than −3.5, less than −4, or even lower).

110. The compound of any of claims 84-109, or a pharmaceutically acceptable salt or ester thereof, wherein T^{A} is T^{A1}, T^{A1}-L^{B}-, T^{A1}-L^{B}-(heteroalkylene), or T^{A1}-L^{B}-(heteroalkylene)-L^{B}-,
wherein L^{B} at each occurrence is independently —SO₂—, —C(═O)—, or a moiety selected from: 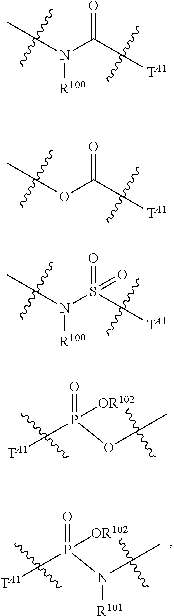
wherein R¹⁰⁰, R¹⁰¹ and R¹⁰² at each occurrence is independently hydrogen, optionally substituted alkyl, or optionally substituted cycloalkyl,
wherein T^{A1} is characterized as having a structure of: 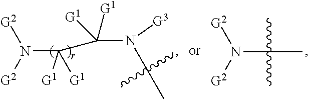
wherein G¹ at each occurrence is independently hydrogen, halogen, optionally substituted C₁₋₄ alkyl, or optionally substituted C₁₋₄ heteroalkyl (such as optionally substituted C₁₋₄ alkoxy), or two gem G¹ group can join to form an —O, ═NH, or ═N(C₁₋₄ alkyl), or two or more G¹ groups can join to form a ring structure,
the integer r is 1-10, such as 1 or 2,
G² at each occurrence is independently hydrogen, G¹⁰, C(O)-G¹⁰, SO₂G¹⁰, C(O)—NH-G¹⁰, or SO₂NHG¹⁰, C(O)—O-G¹⁰, or SO₂₀G¹⁰, wherein G¹⁰ at each occurrence is independently an optionally substituted alkyl, an optionally substituted heteroalkyl, or an optionally substituted 3-10 membered ring structure, which can be carbocyclic, heterocyclic, aromatic, heteroaromatic, or a combination thereof,
G³ is hydrogen or an optionally substituted alkyl (e.g., C₁₋₄ alkyl);
or two G² or one G² and one G¹ or one G¹ and G³ can be joined to form a ring structure such as a 3-10 membered ring structure,
wherein the fragment 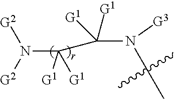
is further characterized in that (i) when the CG¹G¹ group next to the NG³ is not C(═O), the corresponding compound 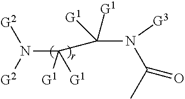
has a cLogP less than 1, preferably, less than 0 (e.g., less than −1, less than −2, less than −3, less than −3.5, less than −4, or even lower), or (ii) when the CG¹G¹ group next to the NG³ is C(═O), the corresponding compound 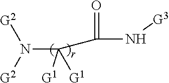
has a cLogP less than 1, preferably, less than 0 (e.g., less than −1, less than −2, less than −3, less than −3.5, less than −4, or even lower), and 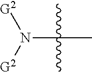
wherein the fragment is further characterized in that (i) when the nitrogen is a basic nitrogen, the corresponding compound (G²)₂N—C(O)—CH₃ has a cLogP less than 1, preferably, less than 0 (e.g., less than −1, less than −2, less than −3, less than −3.5, less than −4, or even lower), or (ii) when the nitrogen is a nonbasic nitrogen, the corresponding compound (G²)₂N—H has a cLogP less than 1, preferably, less than 0 (e.g., less than −1, less than −2, less than −3, less than −3.5, less than −4, or even lower).

111. The compound of any of claims 84-109, or a pharmaceutically acceptable salt or ester thereof, wherein T^{A} is T^{A1}, T^{A1}-L^{B}-, T^{A1}-L^{B}-(heteroalkylene), or T^{A1}-L^{B}-(heteroalkylene)-L^{B}-,
wherein L^{B} at each occurrence is independently —SO₂—, —C(═O)—, or a moiety selected from: 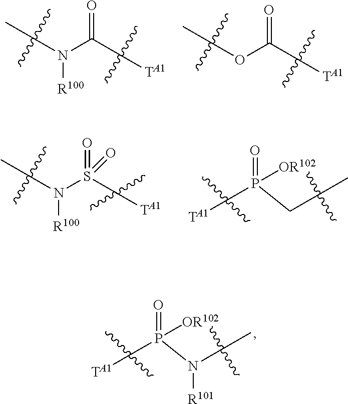
wherein R¹⁰⁰, R¹⁰¹ and R¹⁰² at each occurrence is independently hydrogen, optionally substituted alkyl, or optionally substituted cycloalkyl,
wherein T^{A1} is characterized as having a structure of: 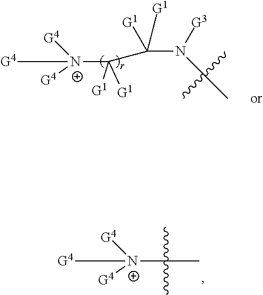
charge balanced with a counterion (e.g., described herein) as necessary,
wherein G¹ at each occurrence is independently hydrogen, halogen, optionally substituted C₁₋₄ alkyl, or optionally substituted C₁₋₄ heteroalkyl (such as optionally substituted C₁₋₄ alkoxy), or two gem G¹ group can join to form an ═O, ═NH, or ═N(C₁₋₄ alkyl), or two or more G¹ groups can join to form a ring structure,
the integer r is 1-10, such as 1 or 2,
G³ is hydrogen or an optionally substituted alkyl (e.g., C₁₋₄ alkyl);
G⁴ at each occurrence is independently hydrogen or G¹⁰, wherein G¹⁰ at each occurrence is independently an optionally substituted alkyl, an optionally substituted heteroalkyl, or an optionally substituted 3-10 membered ring structure, which can be carbocyclic, heterocyclic, aromatic, heteroaromatic, or a combination thereof, or two G⁴ or one G⁴ and one G¹ or one G¹ and G³ can be joined to form an optionally substituted 3-10 membered ring structure,
wherein the fragment 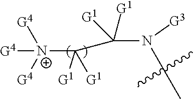
is characterized in that (i) when the CG¹G¹ group next to the NH is not C(═O), the corresponding compound 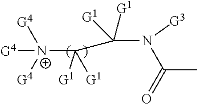
has a cLogP less than 1, preferably, less than 0 (e.g., less than −1, less than −2, less than −3, less than −3.5, less than −4, or even lower), or (ii) when the CG¹G¹ group next to the NH is C(═O), the corresponding compound 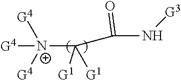
has a cLogP less than 1, preferably, less than 0 (e.g., less than −1, less than −2, less than −3, less than −3.5, less than −4, or even lower).

112. The compound of claim 110 or 111, or a pharmaceutically acceptable salt or ester thereof, wherein G³ is hydrogen and/or G¹ at each occurrence is hydrogen.

113. The compound of any of claims 84-112, or a pharmaceutically acceptable salt or ester thereof, wherein T^{A} is characterized as having a charged group (including zwitterion structures) or a group that can become charged at pH 7, such as primary amine, secondary amine, tertiary amine, quaternary amine, carboxylic acid, etc.

114. The compound of any of claims 84-113, or a pharmaceutically acceptable salt or ester thereof, wherein T^{A} is characterized as having at least two hydrogen bond donors.

115. The compound of any of claims 84-114, or a pharmaceutically acceptable salt or ester thereof, wherein T^{A} is characterized as having at least two hydrogen bond acceptors.

116. The compound of any of claims 84-115, or a pharmaceutically acceptable salt or ester thereof, wherein the covalent bond(s) between the terminal atom(s) of T^{A} and the first end atom of L^{A} is an amide bond.

117. The compound of any of claims 84-115 or a pharmaceutically acceptable salt or ester thereof, wherein the covalent bond(s) between the terminal atom(s) of T^{A} and the first end atom of L^{A} is a non-amide carbon-nitrogen bond, an ester bond, a non-ester carbon-oxygen bond, a carbon-carbon bond, or a carbon-sulfur bond.

118. The compound of any of claims 84-117, or a pharmaceutically acceptable salt or ester thereof, wherein L^{A} is (X) m, wherein X at each occurrence is independently CR₂, C(═O), —C(R)—C(R)—, 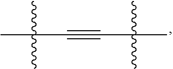 [SMILES: *C#C*]
SiR₂, O, S, SO₂, NR, [NR₂]⁺, or a ring structure, preferably 3-10 membered ring structure, wherein R at each occurrence is independently hydrogen, halogen, optionally substituted C₁₋₄ alkyl, or optionally substituted C₁₋₄ alkoxy, typically R is hydrogen or C₁₋₄ alkyl, and the integer m is at least 10, such as at least 12, at least 14, at least 16, at least 18, at least 20, at least 50, such as 12-50, 16-50, etc., preferably, when L^{N} is null, the X group that is directly bonded with the amide nitrogen in Formula III or III-B is CR₂, preferably, CH₂.

119. The compound of any of claims 84-117, or a pharmaceutically acceptable salt or ester thereof, wherein L^{A} is —(X)ₘ₋₁—C(O)—, wherein the C(O) end is bonded with T^{A}, wherein X at each occurrence is independently CR₂, C(═O), —C(R)═C(R)—, 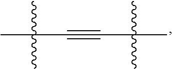 [SMILES: *C#C*]
SiR₂, O, S, SO₂, NR, [NR₂]⁺ or a ring structure, preferably 3-10 membered ring structure, provided that the end X group is CR₂ or a ring structure, preferably 3-10 membered ring structure, wherein R at each occurrence is independently hydrogen, halogen, optionally substituted C₁₋₄ alkyl, or optionally substituted C₁₋₄ alkoxy, typically R is hydrogen or C₁₋₄ alkyl, and the integer m is at least 10, such as at least 12, at least 14, at least 16, at least 18, at least 20, at least 50, such as 12-50, 16-50, etc., and the hydrophobicity of —(X)ₘ₋₁—C(O)— is characterized in that the corresponding compound H—(X)ₘ₋₁—COOH has a cLogP of at least 3, such as between 3-15, preferably, at least 4, preferably, when L^{N} is null, the X group that is directly bonded with the amide nitrogen in Formula III or III-B is CR₂, preferably, CH₂.

120. The compound of any of claims 84-119, or a pharmaceutically acceptable salt or ester thereof, wherein L^{A} is —C₁₂₋₃₀ alkylene- or —C₁₂₋₃₀ alkylene-C(O)—, wherein the —C₁₂₋₃₀ alkylene- is optionally substituted, wherein the optional substituents can optionally be joined together to form a double bond, triple bond, or a ring structure, preferably, wherein the end carbon atoms of the —C₁₂₋₃₀ alkylene- are not substituted with oxo (═O).

121. The compound of any of claims 84-120, or a pharmaceutically acceptable salt or ester thereof, wherein L^{A} or L^{N}-L^{A} has a structure of 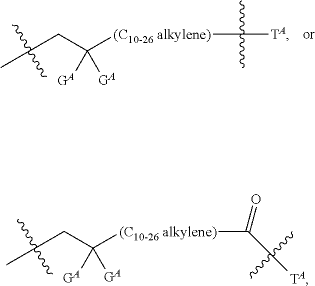
wherein the C₁₀₋₂₆ alkylene is optionally substituted, and wherein G^{A} at each occurrence is independently hydrogen or an optionally substituted C₁₋₄ alkyl, or two G^{A} are joined to form a 3-6 membered ring, such as a cyclopropyl or cyclobutyl ring, preferably, G^{A} at each occurrence is methyl.

122. The compound of any of claims 84-119, or a pharmaceutically acceptable salt or ester thereof, wherein L^{A} is a 12-30 membered heteroalkylene or -(12-30 membered heteroalkylene)-C(O)—, wherein the 12-30 membered heteroalkylene isoptionally substituted and contains 1-6 heteroatoms independently selected from O, N, and S, wherein the sulfur atom(s), if present, is optionally oxidized, wherein the optional substituents can optionally be joined together to form a double bond, triple bond, or a ring structure, preferably, the end atom of the 12-30 membered heteroalkylene that forms a covalent bond with L^{N}(when L^{N} is null, it should be understood that the covalent bond is formed with the amide nitrogen atom in Formula III or III-B) is a carbon atom of a non-carbonyl group.

123. The compound of any of claims 84-119, or a pharmaceutically acceptable salt or ester thereof, wherein L^{A} is 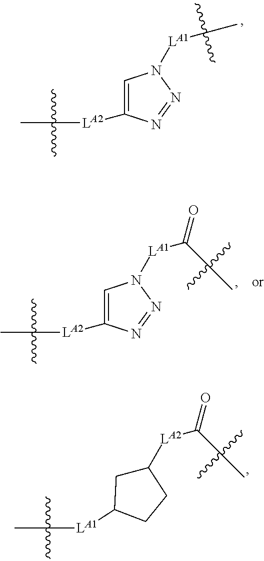
wherein the carbonyl end is bonded with T^{A}, wherein L^{A1} and L^{A2} are each independently a bond, an optionally substituted —C₁₋₃₀ alkylene-, or an optionally substituted-C₁₋₃₀ heteroalkylene-containing 1-6 heteroatoms independently selected from O, N, and S, wherein the sulfur atom(s), if present, is optionally oxidized, wherein the optional substituents can optionally be joined together to form a double bond, triple bond, or a ring structure, provided that the longest chain length of L^{A} is at least that of —(CH₂)₁₂, such as at least that of —(CH₂)₁₄—, at least that of —(CH₂)₁₆—, at least that of —(CH₂)₁₈—, preferably, the end atom of the L^{A1} or L^{A2} that forms a covalent bond with T^{A} is not C of a C(O) or S of a SO₂ group, and the end atom of the L^{A1} or L^{A2} that forms a covalent bond with L^{N}(when L^{N} is null, it should be understood that the covalent bond is formed with the amide nitrogen atom in Formula III or III-B) is a carbon atom of a non-carbonyl group.

124. The compound of any of claims 84-119, or a pharmaceutically acceptable salt or ester thereof, wherein L^{A} or L^{N}-L^{A} has a structure of 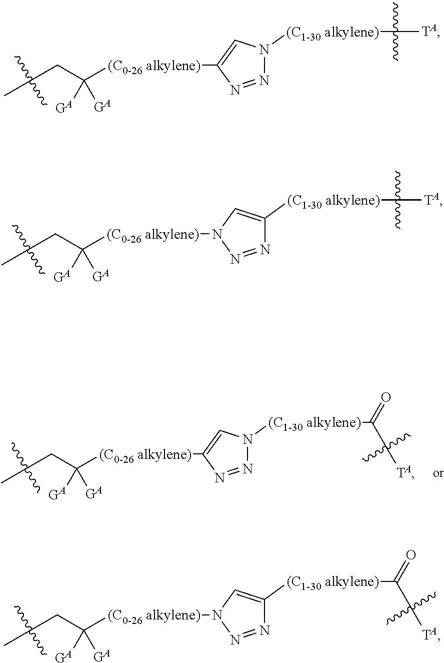
wherein the C₀₋₂₆ alkylene, or C₁₋₃₀ alkylene is optionally substituted, and wherein G^{A} at each occurrence is independently hydrogen or an optionally substituted C₁₋₄ alkyl, or two G^{A} are joined to form a 3-6 membered ring, such as a cyclopropyl or cyclobutyl ring.

125. The compound of any of claims 84-124, or a pharmaceutically acceptable salt or ester thereof, wherein T^{A} is T^{A1} or T^{A1}—C(O)—, wherein T^{A1} is 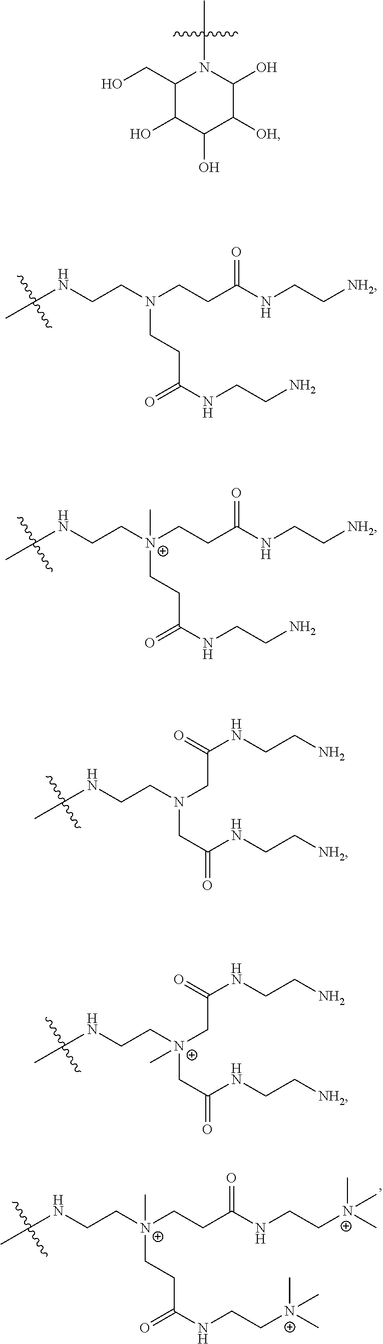 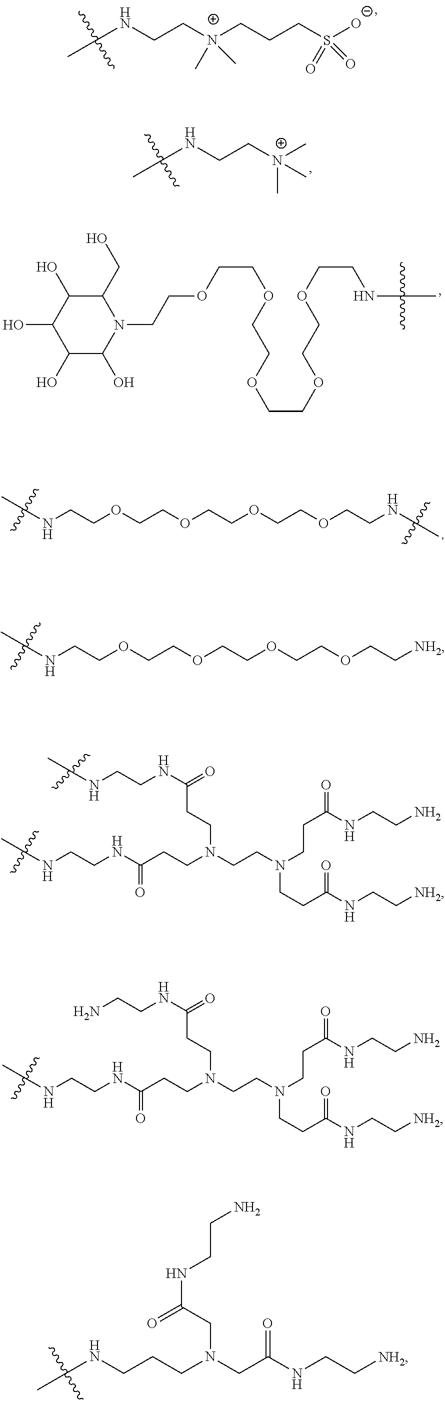 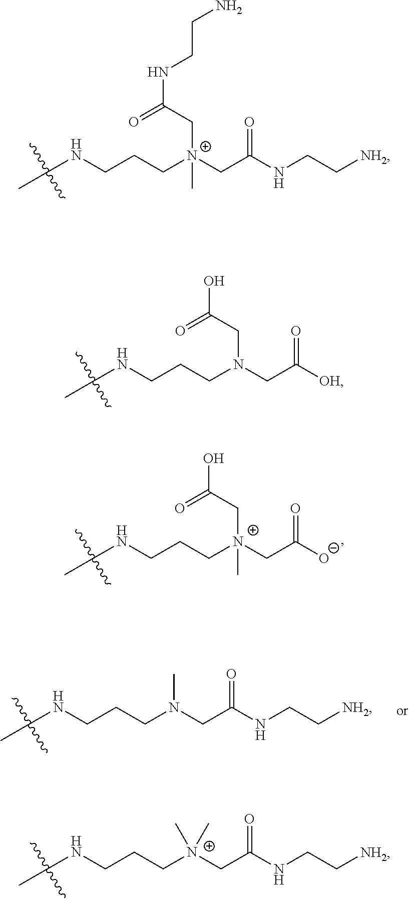
wherein T^{A} is charge balanced with a counterion (e.g., described herein) as necessary.

126. A compound selected from any of the compounds in Table 1 herein, or a compound according to Examples 1-221 herein, or a pharmaceutically acceptable salt or ester thereof.

127. A pharmaceutical composition comprising the compound of any of claims 1-126 or a pharmaceutically acceptable salt or ester thereof and optionally a pharmaceutically acceptable carrier.

128. A method of treating or preventing a disorder, condition or disease that may be responsive to the agonism of the G-protein-coupled receptor 40 in a subject in need thereof comprising administration of a therapeutically effective amount of the compound of any of claims 1-126 or a pharmaceutically acceptable salt or ester thereof, or the pharmaceutical composition of claim 127.

129. A method of treating type 2 diabetes mellitus in a subject in need of treatment comprising administering to the subject a therapeutically effective amount of the compound of any of claims 1-126 or a pharmaceutically acceptable salt or ester thereof, or the pharmaceutical composition of claim 127.

130. The method of claim 128 or 129, further comprising administering to the subject one or more additional therapeutic agents.

131. The method of claim 130, wherein the one or more additional therapeutic agents are selected from PPAR gamma agonists and partial agonists; biguanides; protein tyrosine phosphatase-1B (PTP-1B) inhibitors; dipeptidyl peptidase IV (DPP-IV) inhibitors; insulin or an insulin mimetic; sulfonylureas; a-glucosidase inhibitors; agents which improve a patient's lipid profile, said agents being selected from the group consisting of (i) HMG-COA reductase inhibitors, (ii) bile acid sequestrants, (iii) nicotinyl alcohol, nicotinic acid or a salt thereof, (iv) PPARa agonists, (v) cholesterol absorption inhibitors, (vi) acyl CoA: cholesterol acyltransferase (ACAT) inhibitors, (vii) CETP inhibitors, (viii) PCSK9 inhibitor or antibodies; (ix) apolipoproteins inhibitors; (x) phenolic anti-oxidants; PPARα/γ dual agonists; PPARδ agonists; PPAR α/δ partial agonists; antiobesity compounds; ileal bile acid transporter inhibitors; anti-inflammatory agents; glucagon receptor antagonists; glucokinase activators; GLP-1 and GLP-1 analogs; GLP-1 receptor agonists (peptide and small-molecule); GLP-1/GIP receptor dual agonists; GLP-1/glucagon receptor dual agonists; GLP-1/GIP/insulin receptor triple agonists; GLP-1/GIP/glucagon receptor triple agonists; GIP receptor antibody; GLP-1 analog/GIP receptor antibody; PYY analog; amylin analogs; GPR119 agonist; TGR5 agonist; SSTR3 and/or SSTR5 antagonist or inverse agonist; THRβ agonists; HSD-1 inhibitors; HSD-17 inhibitors and degraders; PNPLA3 inhibitors and degraders; SGLT-2 inhibitors; SGLT-1/SGLT-2 inhibitors; enteric alpha-glucosidase inhibitors; FXR agonists; DGAT1 and/or DGAT2 inhibitors; FGF19 and analogs; FGF21 and analogs; GDF15 and analogs; ANGPTL3 antibody or inhibitor; ANGPTL3/8 antibody; ANGPTL4 inhibitor; Oxyntomodulin; (xi) anti-amyloid beta antibody; (xii) anti-inflammatory agents including but not limited to PDE4 inhibitors, JAK inhibitors, TYK2 inhibitors, SIP receptor modulators, NLRP3 inhibitors, BTK inhibitors, IRAK1 inhibitors, IRAK4 inhibitors, glucocorticoids, anti-TNFα antibodies, anti-IL-12/IL-23 antibodies, (xiii) anti-integrin antibodies including anti-α4β7, anti-α4, anti-β7, anti-MAdCAM-1.

132. Provided herein are compounds, pharmaceutical compositions, and methods of using related to GPR40. The compounds herein are typically GPR40 agonists, which can be used for treating a disorder, condition or disease such as Type 1 or 2 diabetes, obesity, hyperglycemia, glucose intolerance, insulin resistance, hyperinsulinemia, hypercholesterolemia, hypertension, hyperlipoproteinemia, hyperlipidemia, myocardial infarction, stroke, hypertriglyceridemia, dyslipidemia, metabolic syndrome, syndrome X, cardiovascular disease, atherosclerosis, kidney disease, diabetic kidney disease, ketoacidosis, thrombotic disorders, nephropathy, diabetic neuropathy, diabetic retinopathy, sexual dysfunction, dermatopathy, dyspepsia, hypoglycemia, cancer, edema, nonalcoholic steatohepatitis (NASH), lipodystrophy, Prader Willi syndrome, inflammatory bowel diseases including Crohn's disease and ulcerative colitis, irritable bowel syndrome, short bowel syndrome, lymphocytic colitis, rare microscopic colitis, and/or neurodegenerative diseases including but not limited to Alzheimer's disease, Parkinson's disease, amyotrophic lateral sclerosis.
